(19) United States
(12) Patent Application Publication
(10) Pub. No.: US 2022/0339270 A1
(43) Pub. Date: Oct. 27, 2022

(54) COMPOSITIONS AND METHODS FOR THE TREATMENT OF TAUOPATHY

(71) Applicant: Voyager Therapeutics, Inc., Cambridge, MA (US)

(72) Inventors: Wencheng Liu, Cambridge, MA (US); Todd Carter, Cambridge, MA (US); Jinzhao Hou, Cambridge, MA (US); Yanqun Shu, Cambridge, MA (US); Giridhar Murlidharan, Malden, MA (US); Martin Goulet, Weston, MA (US); Dinah Wen-Yee Sah, Hopkinton, MA (US); Steven Paul, Cambridge, MA (US); Xiao-Qin Ren, Cambridge, MA (US); Xin Wang, Arlington, MA (US); Hiu Yan Chung, Cambridge, MA (US)

(73) Assignee: Voyager Therapeutics, Inc., Cambridge, MA (US)

(21) Appl. No.: 17/607,141

(22) PCT Filed: Apr. 29, 2020
(86) PCT No.: PCT/US2020/030357

§ 371 Date: Oct. 28, 2021

Related U.S. Application Data
(60) Provisional application No. 63/002,011, filed on Mar. 30, 2020.
(60) Provisional application No. 62/839,891, filed on Apr. 29, 2019.

(51) Int. Cl. A61K 39/00; A61P 25/28; C12N 7/00; C12N 15/86

(52) U.S. Cl. CPC A61K 39/0007; A61P 25/28; C12N 7/00; C12N 15/86; A61K 2039/5256; C12N 2830/008; C12N 2750/14142; C12N 2750/14145; C12N 2750/14123; C12N 2750/14134

(57) ABSTRACT

The disclosure provides compositions and methods for the preparation, manufacture and therapeutic use of viral vectors, such as adeno-associated virus (AAV) particles having viral genomes encoding one or more antibodies or antibody fragments or antibody-like polypeptides, for the prevention and/or treatment of diseases and/or disorders.

### REFERENCE TO RELEVANT APPLICATIONS

This application claims the benefit of U.S. Provisional Patent Application No. 62/839,891, entitled “Compositions and Methods for the Treatment of Tauopathy”, filed Apr. 29, 2019 and U.S. Provisional Patent Application No. 63/002,011, entitled “Compositions and Methods for the Treatment of Tauopathy” filed Mar. 30, 2020; the contents of each of which are herein incorporated by reference in their entirety.

### REFERENCE TO THE SEQUENCE LISTING

The present application is being filed along with a Sequence Listing in electronic format. The Sequence Listing file, entitled 20571304PCT.txt, was created on Apr. 29, 2020, and is 18,423,475 bytes in size. The information in electronic format of the Sequence Listing is incorporated herein by reference in its entirety.

### FIELD OF THE DISCLOSURE

The disclosure relates to compositions and methods for vectored antibody delivery (VAD), wherein the antibody may be an anti-tau antibody.

### BACKGROUND

Tauopathies are a group of neurodegenerative diseases characterized by the dysfunction and/or aggregation of the microtubule associated protein tau. Tau is normally a very soluble protein known to associate with microtubules based on the extent of its phosphorylation. Tau is considered a critical component of intracellular trafficking processes, particularly in neuronal cells, given their unique and extended structure. Hyperphosphorylation of tau depresses its binding to microtubules and microtubule assembly activity. Further, hyperphosphorylation of tau renders it prone to misfolding and aggregation. In tauopathies, the tau becomes hyperphosphorylated, misfolds and aggregates as neurofibrillary tangles (NFT) of paired helical filaments (PHF), twisted ribbons or straight filaments. These NFT are largely considered indicative of impending neuronal cell death and thought to contribute to widespread neuronal cell loss, leading to a variety of behavioral and cognitive deficits.

The first genetically defined tauopathy was described when mutations in the tau gene were shown to lead to an autosomal dominantly inherited tauopathy known as frontotemporal dementia and parkinsonism linked to chromosome 17 (FTDP-17). This was the first causal evidence that changes in tau could lead to neurodegenerative changes in the brain. These molecules are considered to be more amyloidogenic, meaning they are more likely to become hyperphosphorylated and more likely to aggregate into NFT (Hutton, M. et al., 1998. Nature 393(6686):702-5).

Other known tauopathies include, but are not limited to, Alzheimer's disease (AD), Frontotemporal lobar degeneration (FTLD), Frontotemporal dementia, chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis.

Several approaches have been proposed for therapeutically interfering with progression of tau pathology and preventing the subsequent molecular and cellular consequences. Given that NFT are composed of hyperphosphorylated, misfolded and aggregated forms of tau, interference at each of these stages has yielded a set of avidly pursued targets. Introducing agents that limit phosphorylation, block misfolding or prevent aggregation have all generated promising results. Passive and active immunization with late stage anti-phospho-tau antibodies in mouse models have led to dramatic decreases in tau aggregation and improvements in cognitive parameters. It has also been suggested that introduction of anti-tau antibodies can prevent the trans-neuronal spread of tau pathology.

Antibodies have relatively short half-lives, and this presents an ongoing and long-felt challenge for antibody-based therapies. In order to achieve a sufficiently high concentration of an antibody for long lasting therapeutic effects, antibody therapies are traditionally delivered by repeated administration, e.g. by multiple injections. This dosing regimen results in an inconsistent level of antibody throughout the treatment period, limited efficiency per administration, high cost of administration and consumption of the antibody. Hence, there remains a need in the art for delivery of antibodies and antibody-based therapeutics through alternative routes or modalities of administration.

One such alternative route of administration is by expression vectors (e.g. plasmid or viral vector), including but not limited to, adeno-associated viral vectors (AAVs). Adeno-associated viral vectors are widely used in gene therapy approaches due to a number of advantageous features. As dependoparvoviruses, AAV are non-replicating in infected cells and therefore not associated with any known disease. Further, AAVs may be introduced to a wide variety of host cells, do not integrate into the genome of the host cell, and are capable of infecting both quiescent and dividing cells. AAVs transduce non-replicating and long-lived cells in vivo, resulting in long term expression of the protein of interest. Further, AAVs can be manipulated with cellular and molecular biology techniques to produce non-toxic particles carrying a payload encoded in the AAV viral genome that can be delivered to a target tissue or set of cells with limited or no side-effects. Given the foregoing, the use of AAVs for vectored antibody delivery (VAD) of anti-tau antibodies would allow for longer lasting efficacy, fewer dose treatments, and more consistent levels of the antibody throughout the treatment period.

In vectored antibody delivery (VAD) of anti-tau antibodies, an AAV is used as the delivery modality for a nucleic acid sequence encoding the anti-tau antibody, or a fragment thereof, which results in in vivo expression of the encoded payload, e.g., functional anti-tau antibody, or a fragment thereof.

The mechanism underlying VAD is thought to proceed through the following steps. First, the AAV vector enters the cell via endocytosis, then escapes from the endosomal compartment and is transported to the nucleus wherein the viral genome is released and converted into a double-stranded episomal molecule of DNA by the host. The transcriptionally active episome results in the expression of encoded anti-tau antibodies that may then be secreted from the cell into the circulation. VAD may therefore enable continuous, sustained and long-term delivery of anti-tau antibodies administered by a single injection of AAV particles.

Previous studies of an AAV-mediated antibody technique known as vectored immunoprophylaxis (VIP) have focused on neutralization of human immunodeficiency virus (HIV) (see, e.g. Johnson et al., 2009, Nature Med., 15, 901-906, Saunders et al., 2015, J. Virol., 89(16), 8334-8345, Balasz et al., 2012, Nature 481, 81-84, the contents of which are incorporated herein by reference in their entirety). Balasz et al. reported a long-term, even lifelong, expression of monoclonal antibody at high concentration from a single intramuscular administration in mice that resulted in full protection against HIV infection. AAV-mediated VIP has also been demonstrated against influenza strains (see, e.g. Balasz, et al. Nat. Biotechnol., 2013, 31(7):647-52) and Plasmodium Falciparum, a sporozoite causing malaria infection (see, e.g. Deal at al., 2014, PNAS, 111 (34), 12528-12532), as well as cancer, RSV and drug addiction (see, e.g. review by Schnepp and Johnson, Microbiol. Spectrum 2(4), 2014). Though promising, these studies emphasize efforts to prevent disease. There still remains a need for improved methods of prevention, and new antibody-mediated therapies for research, diagnosis, and treatment of disease.

The present disclosure addresses this need by providing novel AAV particles having viral genomes engineered to encode anti-tau antibodies and antibody-based compositions and methods of using these constructs (e.g., VAD) for the treatment, prevention, diagnosis and research of diseases, disorders and/or conditions associated with tau pathology. The present disclosure further embraces optimized AAV particles for delivery of nucleic acids (e.g., viral genomes) encoding anti-tau antibodies and antibody-based compositions to a subject in need thereof.

### SUMMARY

The present disclosure describes AAV particles for delivery of anti-tau antibodies to a target tissue. AAV particles of the present disclosure may comprise an AAV capsid and a viral genome.

Viral genomes of the present disclosure may comprise a 5′ ITR with a sequence selected from SEQ ID NO: 2076 or 2077, one or more promoter regions with a sequence selected from SEQ ID NO: 2080-2089 and 2238-2239, an antibody polynucleotide with a sequence selected from SEQ ID NO: 1740-1989, 2241-2243 and 2169-2170, or encoding a sequence selected from SEQ ID NO: 1740-1989, 2241-2243 and 2169-2170, a polyadenylation signal sequence with a sequence selected from SEQ ID NO: 2122-2124, and a Y ITR with a sequence selected from SEQ ID NO: 2078-2079. In some embodiments, viral genomes described herein may comprise one or more exon sequences with a sequence selected from SEQ ID NO: 2090-2094. In some embodiments, viral genomes described herein may comprise one or more intron sequences with a sequence selected from SEQ ID NO: 2095-2105, 2240 and 2256-2258. In some embodiments, viral genomes described herein may comprise one or signal sequence regions with a sequence selected from SEQ ID NO: 1740, 1741, 1861, 2106-2117 and 2241. In some embodiments, the signal sequence region is derived from an antibody sequence. In some embodiments, viral genomes described herein may comprise one or more tag sequence regions with a sequence selected from SEQ ID NO: 2118-2121 and 2255. In some embodiments, the viral genomes described herein may comprise a filler sequence region with a sequence selected from SEQ ID NO: 2125-2126. Viral genomes described herein may comprise a sequence selected from SEQ ID NO: 1990-2075, 2137-2168, 2171-2237 and 2260-2321.

Viral genomes described herein may comprise more than one antibody polynucleotide. When more than one antibody polynucleotide exists in a viral genome, these antibody polynucleotides may be separated by a linker sequence, with a sequence selected from SEQ ID NO: 1724-1739, 2244-2254 and 2259. In some embodiments, viral genomes described herein comprise a first antibody polynucleotide sequence and a second antibody polynucleotide sequence, wherein each may encode an antibody heavy or light chain or a fragment thereof. In some embodiments, the viral genome comprises more than two antibody polynucleotides.

In some embodiments, a viral genome, when read 5′ to 3′, may encode an antibody heavy chain, at least one linker, and an antibody light chain. This viral genome may be described as comprising a heavy-linker-light configuration.

In some embodiments, a viral genome, when read 5′ to 3′, may encode an antibody light chain, at least one linker, and an antibody heavy chain. This viral genome may be described as comprising a light-linker-heavy configuration.

The viral genomes described herein may be packaged into an AAV particle comprising any AAV serotype known in the art, or selected from VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2 variants, AAV2G9, AAV3, AAV2/3 variants AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9K449R, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the capsid of the AAV particle is one of VOY101, PHP.B, AAV9, AAV9K449, AAV1, AAV2, VOY201, AAV2 variant or AAV2/3 variant.

AAV particles described herein may be prepared as a pharmaceutical composition. In some embodiments, the pharmaceutical composition may be administered to a subject. In some embodiments, a method of producing a functional antibody in a subject may comprise administration of a pharmaceutical composition described herein to the subject. In some embodiments, the functional antibody may be encoded by one or more antibody polynucleotides of a viral genome described herein, packaged into an AAV particle. In some embodiments, the functional antibody may be encoded by two different viral genomes, packaged into separate AAV particles. The functional antibody may be expressed in a target cell or tissue in a range from 0.001 μg/mL to 100 mg/mL.

Pharmaceutical compositions described herein may be used in a method of treating tauopathy, wherein a therapeutically effective amount of a pharmaceutical composition described herein is administered to a subject in need. The tauopathy may be any one of Alzheimer's disease (AD), frontotemporal dementia and parkinsonism linked to chromosome 17 (FTDP-17), Frontotemporal lobar degeneration (FTLD), Frontotemporal dementia, chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis or other tau associated disease.

Pharmaceutical compositions described herein may be used in a method of preventing tauopathy, wherein a therapeutically effective amount of a pharmaceutical composition described herein is administered to a subject in need. The tauopathy may be any one of Alzheimer's disease (AD), frontotemporal dementia and parkinsonism linked to chromosome 17 (FTDP-17), Frontotemporal lobar degeneration (FTLD). Frontotemporal dementia, chronic traumatic encephalopathy (CTE), Progressive Supranuclear Palsy (PSP), Down's syndrome, Pick's disease. Corticobasal degeneration (CBD), Corticobasal syndrome. Amyotrophic lateral sclerosis (ALS), Prion diseases, Creutzfeldt-Jakob disease (CJD), Multiple system atrophy, Tangle-only dementia, and Progressive subcortical gliosis or other tau associated disease.

The method of delivery of the pharmaceutical composition described herein to a subject in need may comprise any delivery route known in the art, including, intravenous (into a vein), intramuscular (into a muscle), intraparenchymal (into the substance of a tissue, e.g., brain), enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal, (into the skin itself), subcutaneous (under the skin), nasal administration (through the nose), intravenous bolus, intravenous drip, intra-arterial (into an artery), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

In some embodiments AAV particles or pharmaceutical compositions described herein are delivered by intramuscular administration. In some embodiments AAV particles or pharmaceutical compositions described herein are delivered by intravascular administration. In some embodiments AAV particles or pharmaceutical compositions described herein are delivered by intravenous administration. In some embodiments AAV particles or pharmaceutical compositions described herein are delivered by intracerebroventricular administration. In some embodiments AAV particles or pharmaceutical compositions described herein are delivered by intraparenchymal administration. In some embodiments AAV particles or pharmaceutical compositions described herein are delivered by intrathecal administration.

### DETAILED DESCRIPTION

### I. Compositions

According to the present disclosure, compositions for delivering functional anti-tau antibodies and/or antibody-based compositions by adeno-associated viruses (AAVs) are provided. AAV particles may be provided via any of several routes of administration, to a cell, tissue, organ, or organism, in vivo, ex vivo, or in vitro.

As used herein, an “AAV particle” is a virus which comprises a viral genome with at least one payload region and at least one inverted terminal repeat (ITR) region.

As used herein, “viral genome” or “vector genome” refers to the nucleic acid sequence(s) encapsulated in an AAV particle. Viral genomes comprise at least one payload region encoding polypeptides, e.g., antibodies, antibody-based compositions or fragments thereof.

As used herein, a “payload” or “payload region” is any nucleic acid molecule which encodes one or more polypeptides. At a minimum, a payload region comprises nucleic acid sequences that encode an antibody, an antibody-based composition, or a fragment thereof, but may also optionally comprise one or more functional or regulatory elements to facilitate transcriptional expression and/or polypeptide translation.

As used herein, “VL” and “VH” refer to components of a light chain or heavy chain of an antibody, respectively, or a fragment thereof. In some embodiments, “VL” and “VH” refer to the variable regions of the light or heavy chain of an antibody, respectively, or a fragment thereof. In another embodiment, “VL” and “VH” may also embrace a constant region of a light or heavy chain of an antibody, or a fragment thereof. In another embodiment, “VL” and “VH” may embrace the entirety of an antibody light chain or heavy chain, respectively.

In some embodiments, AAV particles, viral genomes and/or payloads, and the methods of their use may be as described in WO2017189963, the contents of which are herein incorporated by reference in their entirety.

The nucleic acid sequences and polypeptides disclosed herein may be engineered to contain modular elements and/or sequence motifs assembled to enable expression of the antibodies or antibody-based compositions. In some embodiments, the nucleic acid sequence comprising the payload region may comprise one or more of a promoter region, an intron, a Kozak sequence, an enhancer, or a polyadenylation sequence. Payload regions typically encode antibodies or antibody-based compositions, which may include an antibody heavy chain domain, an antibody light chain domain, both antibody heavy and light chain domains, or fragments of the foregoing in combination with each other or in combination with other polypeptide moieties. In some cases, payload regions may also encode one or more linkers or joining regions between antibody heavy and light chain domains or fragments. The order of expression, structural position, or concatemer count (heavy chain, light chain, or linker) may be different within or among different payload regions. The identity, position and number of linkers expressed by payload regions may also vary.

The payload regions may be delivered to one or more target cells, tissues, organs, or organisms within the viral genome of an AAV particle.

### Adeno-Associated Viruses (AAVs) and AAV Particles

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV vector genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, AAV particles of the present disclosure are recombinant AAV viral vectors which are replication defective and lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ, or an organism.

In some embodiments, the viral genome of the AAV particles of the present disclosure comprise at least one control element which provides for the replication, transcription, and translation of a coding sequence encoded therein. Not all of the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes, scAAV vector genomes contain DNA strands which anneal together to form double stranded DNA. By skipping second strand synthesis, scAAVs allow for rapid expression in the transduced cell.

In some embodiments, the AAV particle of the present disclosure is an scAAV.

In some embodiments, the AAV particle of the present disclosure is an ssAAV.

Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In some embodiments, the capsids of the AAV particles are engineered according to the methods described in US Publication Number US20130195801, the contents of which are incorporated herein by reference in their entirety.

In some embodiments, the AAV particles comprising a payload region encoding the polypeptides may be introduced into mammalian cells.

### AAV Serotypes

AAV particles of the present disclosure may comprise or be derived from any natural or recombinant AAV serotype. According to the present disclosure, the AAV particles may utilize or be based on a serotype or include a peptide selected from any of the following VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9K449R, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the AAV serotype may be, or have, a sequence as described in United States Publication No. US20030138772, the contents of which are herein incorporated by reference in their entirety, such as, but not limited to: AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to CySR1, CySR2, CySR3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gin) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAVS-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.5 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO:10 of US20150376607), AAV-LK10 (SEQ ID NO:11 of US20150376607), AAV-LK11 (SEQ ID NO:12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV serotype may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV serotype may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAV capsid serotype selection or use may be from a variety of species. In some embodiments, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In some embodiments, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F4111), AAV9.9 (G1203A. G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L5111, L537M), AAV9.55 (T1605A; F535L), AAV9.58 (C1475T, C1579A; T4921, H527N), AAV.59 (T1336C; Y446H), AAV9.61 (A1493T; N4981), AAV9.64 (C1531A, A1617T; L5111), AAV9.65 (C1335T, T1530C, C1568A; A523D), AAV9.68 (C1510A; P504T), AAV9.80 (G1441A, G481R), AAV9.83 (C1402A. A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO; 2 and 20 of WO2016049230), AAVF2/HSC2 (SEQ ID NO; 3 and 21 of WO2016049230), AAVF3/HSC3 (SEQ ID NO; 5 and 22 of WO2016049230), AAVF4/HSC4 (SEQ ID NO; 6 and 23 of WO2016049230), AAVF5/HSC5 (SEQ ID NO; 11 and 25 of WO2016049230), AAVF6/HSC6 (SEQ ID NO; 7 and 24 of WO2016049230), AAVF7/HSC7 (SEQ ID NO; 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In some embodiments, the AAV serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV CLv-D1 (SEQ ID NO: 22 and 96 of U.S. Pat. No. 8,734,809), AAV CLv-D2 (SEQ ID NO: 23 and 97 of U.S. Pat. No. 8,734,809), AAV CLv-D3 (SEQ ID NO: 24 and 98 of U.S. Pat. No. 8,734,809), AAV CLv-D4 (SEQ ID NO: 25 and 99 of U.S. Pat. No. 8,734,809), AAV CLv-D5 (SEQ ID NO: 26 and 100 of U.S. Pat. No. 8,734,809), AAV CLv-D6 (SEQ ID NO: 27 and 101 of U.S. Pat. No. 8,734,809), AAV CLv-D7 (SEQ ID NO: 28 and 102 of U.S. Pat. No. 8,734,809), AAV CLv-D8 (SEQ ID NO: 29 and 103 of U.S. Pat. No. 8,734,809), AAV CLv-E1 (SEQ ID NO: 13 and 87 of U.S. Pat. No. 8,734,809), AAV CLv-R1 (SEQ ID NO: 30 and 104 of U.S. Pat. No. 8,734,809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: X and X of U.S. Pat. No. 8,734,809), AAV CLv-R9 (SEQ ID NO: X and X of U.S. Pat. No. 8,734,809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of U.S. Pat. No. 8,734,809), AAV CLg-F4 (SEQ ID NO: 42 and 116 of U.S. Pat. No. 8,734,809), AAV CLg-F5 (SEQ ID NO: 43 and 117 of U.S. Pat. No. 8,734,809), AAV CLg-F6 (SEQ ID NO: 43 and 117 of U.S. Pat. No. 8,734,809), AAV CLg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of U.S. Pat. No. 8,734,809), AAV CSp-10 (SEQ ID NO: 46 and 120 of U.S. Pat. No. 8,734,809), AAV CSp-11 (SEQ ID NO: 47 and 121 of U.S. Pat. No. 8,734,809), AAV CSp-2 (SEQ ID NO: 48 and 122 of U.S. Pat. No. 8,734,809), AAV CSp-3 (SEQ ID NO: 49 and 123 of U.S. Pat. No. 8,734,809), AAV CSp-4 (SEQ ID NO: 50 and 124 of U.S. Pat. No. 8,734,809), AAV CSp-6 (SEQ ID NO: 51 and 125 of U.S. Pat. No. 8,734,809), AAV CSp-7 (SEQ ID NO: 52 and 126 of U.S. Pat. No. 8,734,809), AAV CSp-8 (SEQ ID NO: 53 and 127 of U.S. Pat. No. 8,734,809), AAV CSp-9 (SEQ ID NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of U.S. Pat. No. 8,734,809), AAV CKd-3 (SEQ ID NO: 60 and 134 of U.S. Pat. No. 8,734,809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of U.S. Pat. No. 8,734,809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809), AAV CLv-3 (SEQ ID NO: 69 and 143 of U.S. Pat. No. 8,734,809), AAV CLv-4 (SEQ ID NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ ID NO: 77 and 151 of U.S. Pat. No. 8,734,809), AAV CKd-B6 (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809), AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of U.S. Pat. No. 8,734,809), AAV CKd-H5 (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of U.S. Pat. No. 8,734,809), AAV CLv1-1 (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U.S. Pat. No. 8,734,809), AAV.VR-355 (SEQ ID NO: 181 of U.S. Pat. No. 8,734,809), AAV.hu.48R3 (SEQ ID NO: 183 of U.S. Pat. No. 8,734,809), or variants or derivatives thereof.

In some embodiments, the AAV serotype may be, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7.10 (SEQ ID NO: 11 and 61 of WO2016065001), AAV CKd-N3 (SEQ ID NO: 12 and 62 of WO2016065001), AAV CKd-N4 (SEQ ID NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In some embodiments, the AAV particle may have, or may be a serotype selected from any of those found in Table 1.

In some embodiments, the AAV capsid may comprise a sequence, fragment or variant thereof, of any of the sequences in Table 1.

In some embodiments, the AAV capsid may be encoded by a sequence, fragment or variant as described in Table 1.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine). N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gin) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine. U (Sec) for Selenocysteine, X (Xaa) for any amino acid; and Z (Glx) for Glutamine or Glutamic acid.

TABLE 1
AAV Serotypes

| | SEQ | |
|---|---|---|
| Serotype | ID NO: | Reference Information |
| VOY101 | 1 | — |
| VOY101 | 2 | — |
| VOY201 | 3 | — |
| VOY201 | 4534 | — |
| PHP.N/PHP.B-DGT | 4 | WO2017100671 SEQ ID NO: 46 |
| AAVPHP.B or G2B-26 | 5 | WO2015038958 SEQ ID NO: 8 and 13 |
| AAVPHP.B | 6 | WO2015038958 SEQ ID NO: 9 |
| AAVG2B-13 | 7 | WO2015038958 SEQ ID NO: 12 |
| AAVTH1.1-32 | 8 | WO2015038958 SEQ ID NO: 14 |
| AAVTH1.1-35 | 9 | WO2015038958 SEQ ID NO: 15 |
| PHP.S/G2A12 | 10 | WO2017100671 SEQ ID NO: 47 |
| AAV9/hu.14 K449R | 11 | WO2017100671 SEQ ID NO: 45 |
| AAV1 | 12 | US20150159173 SEQ ID NO: 11, US20150315612 SEQ ID NO: 202 |
| AAV1 | 13 | US20160017295 SEQ ID NO: 1, US20030138772 SEQ ID NO: 64, |
| | | US20150159173 SEQ ID NO: 27, US20150315612 SEQ ID NO: 219, |
| | | U.S. Pat. No. 7,198,951 SEQ ID NO: 5 |
| AAV1 | 14 | US20030138772 SEQ ID NO: 6 |
| AAV1.3 | 15 | US20030138772 SEQ ID NO: 14 |
| AAV10 | 16 | US20030138772 SEQ ID NO: 117 |
| AAV10 | 17 | WO2015121501 SEQ ID NO: 9 |
| AAV10 | 18 | WO2015121501 SEQ ID NO: 8 |
| AAV11 | 19 | US20030138772 SEQ ID NO: 118 |
| AAV12 | 20 | US20030138772 SEQ ID NO: 119 |
| AAV2 | 21 | US20150159173 SEQ ID NO: 7, US20150315612 SEQ ID NO: 211 |
| AAV2 | 22 | US20030138772 SEQ ID NO: 70, US20150159173 SEQ ID NO: 23, |
| | | US20150315612 SEQ ID NO: 221, US20160017295 SEQ ID NO: 2, |
| | | U.S. Pat. No. 6,156,303 SEQ ID NO: 4, U.S. Pat. No. 7,198,951 SEQ ID NO: 4, |
| | | WO2015121501 SEQ ID NO: 1 |
| AAV2 | 23 | U.S. Pat. No. 6,156,303 SEQ ID NO: 8 |
| AAV2 | 24 | US20030138772 SEQ ID NO: 7 |
| AAV2 | 25 | U.S. Pat. No. 6,156,303 SEQ ID NO: 3 |
| AAV2.5T | 26 | U.S. Pat. No. 9,233,131 SEQ ID NO: 42 |
| AAV223.10 | 27 | US20030138772 SEQ ID NO: 75 |
| AAV223.2 | 28 | US20030138772 SEQ ID NO: 49 |
| AAV223.2 | 29 | US20030138772 SEQ ID NO: 76 |
| AAV223.4 | 30 | US20030138772 SEQ ID NO: 50 |
| AAV223.4 | 31 | US20030138772 SEQ ID NO: 73 |
| AAV223.5 | 32 | US20030138772 SEQ ID NO: 51 |
| AAV223.5 | 33 | US20030138772 SEQ ID NO: 74 |
| AAV223.6 | 34 | US20030138772 SEQ ID NO: 52 |
| AAV223.6 | 35 | US20030138772 SEQ ID NO: 78 |
| AAV223.7 | 36 | US20030138772 SEQ ID NO: 53 |
| AAV223.7 | 37 | US20030138772 SEQ ID NO: 77 |
| AAV29.3 | 38 | US20030138772 SEQ ID NO: 82 |
| AAV29.4 | 39 | US20030138772 SEQ ID NO: 12 |
| AAV29.5 | 40 | US20030138772 SEQ ID NO: 83 |
| AAV29.5 (AAVbb.2) | 41 | US20030138772 SEQ ID NO: 13 |
| AAV3 | 42 | US20150159173 SEQ ID NO: 12 |
| AAV3 | 43 | US20030138772 SEQ ID NO: 71, US20150159173 SEQ ID NO: 28, |
| | | US20160017295 SEQ ID NO: 3, U.S. Pat. No. 7,198,951 SEQ ID NO: 6 |
| AAV3 | 44 | US20030138772 SEQ ID NO: 8 |
| AAV3.3b | 45 | US20030138772 SEQ ID NO: 72 |
| AAV3-3 | 46 | US20150315612 SEQ ID NO: 200 |
| AAV3-3 | 47 | US20150315612 SEQ ID NO: 217 |
| AAV3a | 48 | U.S. Pat. No. 6,156,303 SEQ ID NO: 5 |
| AAV3a | 49 | U.S. Pat. No. 6,156,303 SEQ ID NO: 9 |
| AAV3b | 50 | U.S. Pat. No. 6,156,303 SEQ ID NO: 6 |
| AAV3b | 51 | U.S. Pat. No. 6,156,303 SEQ ID NO: 10 |
| AAV3b | 52 | U.S. Pat. No. 6,156,303 SEQ ID NO: 1 |
| AAV4 | 53 | US20140348794 SEQ ID NO: 17 |
| AAV4 | 54 | US20140348794 SEQ ID NO: 5 |
| AAV4 | 55 | US20140348794 SEQ ID NO: 3 |
| AAV4 | 56 | US20140348794 SEQ ID NO: 14 |
| AAV4 | 57 | US20140348794 SEQ ID NO: 15 |
| AAV4 | 58 | US20140348794 SEQ ID NO: 19 |
| AAV4 | 59 | US20140348794 SEQ ID NO: 12 |
| AAV4 | 60 | US20140348794 SEQ ID NO: 13 |
| AAV4 | 61 | US20140348794 SEQ ID NO: 7 |
| AAV4 | 62 | US20140348794 SEQ ID NO: 8 |
| AAV4 | 63 | US20140348794 SEQ ID NO: 9 |
| AAV4 | 64 | US20140348794 SEQ ID NO: 2 |
| AAV4 | 65 | US20140348794 SEQ ID NO: 10 |
| AAV4 | 66 | US20140348794 SEQ ID NO: 11 |
| AAV4 | 67 | US20140348794 SEQ ID NO: 18 |
| AAV4 | 68 | US20030138772 SEQ ID NO: 63, US20160017295 SEQ ID NO: 4, |
| | | US20140348794 SEQ ID NO: 4 |
| AAV4 | 69 | US20140348794 SEQ ID NO: 16 |
| AAV4 | 70 | US20140348794 SEQ ID NO: 20 |
| AAV4 | 71 | US20140348794 SEQ ID NO: 6 |
| AAV4 | 72 | US20140348794 SEQ ID NO: 1 |
| AAV42.2 | 73 | US20030138772 SEQ ID NO: 9 |
| AAV42.2 | 74 | US20030138772 SEQ ID NO: 102 |
| AAV42.3b | 75 | US20030138772 SEQ ID NO: 36 |
| AAV42.3B | 76 | US20030138772 SEQ ID NO: 107 |
| AAV42.4 | 77 | US20030138772 SEQ ID NO: 33 |
| AAV42.4 | 78 | US20030138772 SEQ ID NO: 88 |
| AAV42.8 | 79 | US20030138772 SEQ ID NO: 27 |
| AAV42.8 | 80 | US20030138772 SEQ ID NO: 85 |
| AAV43.1 | 81 | US20030138772 SEQ ID NO: 39 |
| AAV43.1 | 82 | US20030138772 SEQ ID NO: 92 |
| AAV43.12 | 83 | US20030138772 SEQ ID NO: 41 |
| AAV43.12 | 84 | US20030138772 SEQ ID NO: 93 |
| AAV43.20 | 85 | US20030138772 SEQ ID NO: 42 |
| AAV43.20 | 86 | US20030138772 SEQ ID NO: 99 |
| AAV43.21 | 87 | US20030138772 SEQ ID NO: 43 |
| AAV43.21 | 88 | US20030138772 SEQ ID NO: 96 |
| AAV43.23 | 89 | US20030138772 SEQ ID NO: 44 |
| AAV43.23 | 90 | US20030138772 SEQ ID NO: 98 |
| AAV43.25 | 91 | US20030138772 SEQ ID NO: 45 |
| AAV43.25 | 92 | US20030138772 SEQ ID NO: 97 |
| AAV43.5 | 93 | US20030138772 SEQ ID NO: 40 |
| AAV43.5 | 94 | US20030138772 SEQ ID NO: 94 |
| AAV4-4 | 95 | US20150315612 SEQ ID NO: 201 |
| AAV4-4 | 96 | US20150315612 SEQ ID NO: 218 |
| AAV44.1 | 97 | US20030138772 SEQ ID NO: 46 |
| AAV44.1 | 98 | US20030138772 SEQ ID NO: 79 |
| AAV44.5 | 99 | US20030138772 SEQ ID NO: 47 |
| AAV44.5 | 100 | US20030138772 SEQ ID NO: 80 |
| AAV4407 | 101 | US20150315612 SEQ ID NO: 90 |
| AAV5 | 102 | U.S. Pat. No. 7,427,396 SEQ ID NO: 1 |
| AAV5 | 103 | US20030138772 SEQ ID NO: 114 |
| AAV5 | 104 | US20160017295 SEQ ID NO: 5, U.S. Pat. No. 7,427,396 SEQ ID NO: 2, |
| | | US20150315612 SEQ ID NO: 216 |
| AAV5 | 105 | US20150315612 SEQ ID NO: 199 |
| AAV6 | 106 | US20150159173 SEQ ID NO: 13 |
| AAV6 | 107 | US20030138772 SEQ ID NO: 65, US20150159173 SEQ ID NO: 29, |
| | | US20160017295 SEQ ID NO: 6, U.S. Pat. No. 6,156,303 SEQ ID NO: 7 |
| AAV6 | 108 | U.S. Pat. No. 6,156,303 SEQ ID NO: 11 |
| AAV6 | 109 | U.S. Pat. No. 6,156,303 SEQ ID NO: 2 |
| AAV6 | 110 | US20150315612 SEQ ID NO: 203 |
| AAV6 | 111 | US20150315612 SEQ ID NO: 220 |
| AAV6.1 | 112 | US20150159173 |
| AAV6.12 | 113 | US20150159173 |
| AAV6.2 | 114 | US20150159173 |
| AAV7 | 115 | US20150159173 SEQ ID NO: 14 |
| AAV7 | 116 | US20150315612 SEQ ID NO: 183 |
| AAV7 | 117 | US20030138772 SEQ ID NO: 2, US20150159173 SEQ ID NO: 30, |
| | | US20150315612 SEQ ID NO: 181, US20160017295 SEQ ID NO: 7 |
| AAV7 | 118 | US20030138772 SEQ ID NO: 3 |
| AAV7 | 119 | US20030138772 SEQ ID NO: 1, US20150315612 SEQ ID NO: 180 |
| AAV7 | 120 | US20150315612 SEQ ID NO: 213 |
| AAV7 | 121 | US20150315612 SEQ ID NO: 222 |
| AAV8 | 122 | US20150159173 SEQ ID NO: 15 |
| AAV8 | 123 | US20150376240 SEQ ID NO: 7 |
| AAV8 | 124 | US20030138772 SEQ ID NO: 4, US20150315612 SEQ ID NO: 182 |
| AAV8 | 125 | US20030138772 SEQ ID NO: 95, US20140359799 SEQ ID NO: 1, |
| | | US20150159173 SEQ ID NO: 31, US20160017295 SEQ ID NO: 8, |
| | | U.S. Pat. No. 7,198,951 SEQ ID NO: 7, US20150315612 SEQ ID NO: 223 |
| AAV8 | 126 | US20150376240 SEQ ID NO: 8 |
| AAV8 | 127 | US20150315612 SEQ ID NO: 214 |
| AAV-8b | 128 | US20150376240 SEQ ID NO: 5 |
| AAV-8b | 129 | US20150376240 SEQ ID NO: 3 |
| AAV-8h | 130 | US20150376240 SEQ ID NO: 6 |
| AAV-8h | 131 | US20150376240 SEQ ID NO: 4 |
| AAV9 | 132 | US20030138772 SEQ ID NO: 5 |
| AAV9 | 133 | U.S. Pat. No. 7,198,951 SEQ ID NO: 1 |
| AAV9 | 134 | US20160017295 SEQ ID NO: 9 |
| AAV9 | 135 | US20030138772 SEQ ID NO: 100, U.S. Pat. No. 7,198,951 SEQ ID NO: 2 |
| AAV9 | 136 | U.S. Pat. No. 7,198,951 SEQ ID NO: 3 |
| AAV9 (AAVhu.14) | 137 | U.S. Pat. No. 7,906,111 SEQ ID NO: 3; WO2015038958 SEQ ID NO: 11 |
| AAV9 (AAVhu.14) | 138 | U.S. Pat. No. 7,906,111 SEQ ID NO: 123; WO2015038958 SEQ ID NO: 2 |
| AAVA3.1 | 139 | US20030138772 SEQ ID NO: 120 |
| AAVA3.3 | 140 | US20030138772 SEQ ID NO: 57 |
| AAVA3.3 | 141 | US20030138772 SEQ ID NO: 66 |
| AAVA3.4 | 142 | US20030138772 SEQ ID NO: 54 |
| AAVA3.4 | 143 | US20030138772 SEQ ID NO: 68 |
| AAVA3.5 | 144 | US20030138772 SEQ ID NO: 55 |
| AAVA3.5 | 145 | US20030138772 SEQ ID NO: 69 |
| AAVA3.7 | 146 | US20030138772 SEQ ID NO: 56 |
| AAVA3.7 | 147 | US20030138772 SEQ ID NO: 67 |
| AAV29.3 (AAVbb.1) | 148 | US20030138772 SEQ ID NO: 11 |
| AAVC2 | 149 | US20030138772 SEQ ID NO: 61 |
| AAVCh.5 | 150 | US20150159173 SEQ ID NO: 46, US20150315612 SEQ ID NO: 234 |
| AAVcy.2 (AAV13.3) | 151 | US20030138772 SEQ ID NO: 15 |
| AAV24.1 | 152 | US20030138772 SEQ ID NO: 101 |
| AAVcy.3 (AAV24.1) | 153 | US20030138772 SEQ ID NO: 16 |
| AAV27.3 | 154 | US20030138772 SEQ ID NO: 104 |
| AAVcy.4 (AAV27.3) | 155 | US20030138772 SEQ ID NO: 17 |
| AAVcy.5 | 156 | US20150315612 SEQ ID NO: 227 |
| AAV7.2 | 157 | US20030138772 SEQ ID NO: 103 |
| AAVcy.5 (AAV7.2.) | 158 | US20030138772 SEQ ID NO: 18 |
| AAV16.3 | 159 | US20030138772 SEQ ID NO: 105 |
| AAVcy.6 (AAV16.3) | 160 | US20030138772 SEQ ID NO: 10 |
| AAVcy.5 | 161 | US20150159173 SEQ ID NO: 8 |
| AAVcy.5 | 162 | US20150159173 SEQ ID NO: 24 |
| AAVCy.5R1 | 163 | US20150159173 |
| AAVCy.5R2 | 164 | US20150159173 |
| AAVCy.5R3 | 165 | US20150159173 |
| AAVCy.5R4 | 166 | US20150159173 |
| AAVDJ | 167 | US20140359799 SEQ ID NO: 3, U.S. Pat. No. 7,588,772 SEQ ID NO: 2 |
| AAVDJ | 168 | US20140359799 SEQ ID NO: 2, U.S. Pat. No. 7,588,772 SEQ ID NO: 1 |
| AAVDJ-8 | 169 | U.S. Pat. No. 7,588,772; Grimm et al 2008 |
| AAVDJ-8 | 170 | U.S. Pat. No. 7,588,772; Grimm et al 2008 |
| AAVF5 | 171 | US20030138772 SEQ ID NO: 110 |
| AAVH2 | 172 | US20030138772 SEQ ID NO: 26 |
| AAVH6 | 173 | US20030138772 SEQ ID NO: 25 |
| AAVhE1.1 | 174 | U.S. Pat. No. 9,233,131 SEQ ID NO: 44 |
| AAVhEr1.14 | 175 | U.S. Pat. No. 9,233,131 SEQ ID NO: 46 |
| AAVhEr1.16 | 176 | U.S. Pat. No. 9,233,131 SEQ ID NO: 48 |
| AAVhEr1.18 | 177 | U.S. Pat. No. 9,233,131 SEQ ID NO: 49 |
| AAVhEr1.23 (AAVhEr2.29) | 178 | U.S. Pat. No. 9,233,131 SEQ ID NO: 53 |
| AAVhEr1.35 | 179 | U.S. Pat. No. 9,233,131 SEQ ID NO: 50 |
| AAVhEr1.36 | 180 | U.S. Pat. No. 9,233,131 SEQ ID NO: 52 |
| AAVhEr1.5 | 181 | U.S. Pat. No. 9,233,131 SEQ ID NO: 45 |
| AAVhEr1.7 | 182 | U.S. Pat. No. 9,233,131 SEQ ID NO: 51 |
| AAVhEr1.8 | 183 | U.S. Pat. No. 9,233,131 SEQ ID NO: 47 |
| AAVhEr2.16 | 184 | U.S. Pat. No. 9,233,131 SEQ ID NO: 55 |
| AAVhEr2.30 | 185 | U.S. Pat. No. 9,233,131 SEQ ID NO: 56 |
| AAVhEr2.31 | 186 | U.S. Pat. No. 9,233,131 SEQ ID NO: 58 |
| AAVhEr2.36 | 187 | U.S. Pat. No. 9,233,131 SEQ ID NO: 57 |
| AAVhEr2.4 | 188 | U.S. Pat. No. 9,233,131 SEQ ID NO: 54 |
| AAVhEr3.1 | 189 | U.S. Pat. No. 9,233,131 SEQ ID NO: 59 |
| AAVhu.1 | 190 | US20150315612 SEQ ID NO: 46 |
| AAVhu.1 | 191 | US20150315612 SEQ ID NO: 144 |
| AAVhu.10 (AAV16.8) | 192 | US20150315612 SEQ ID NO: 56 |
| AAVhu.10 (AAV16.8) | 193 | US20150315612 SEQ ID NO: 156 |
| AAVhu.11 (AAV16.12) | 194 | US20150315612 SEQ ID NO: 57 |
| AAVhu.11 (AAV16.12) | 195 | US20150315612 SEQ ID NO: 153 |
| AAVhu.12 | 196 | US20150315612 SEQ ID NO: 59 |
| AAVhu.12 | 197 | US20150315612 SEQ ID NO: 154 |
| AAVhu.13 | 198 | US20150159173 SEQ ID NO: 16, US20150315612 SEQ ID NO: 71 |
| AAVhu.13 | 199 | US20150159173 SEQ ID NO: 32, US20150315612 SEQ ID NO: 129 |
| AAVhu.136.1 | 200 | US20150315612 SEQ ID NO: 165 |
| AAVhu.140.1 | 201 | US20150315612 SEQ ID NO: 166 |
| AAVhu.140.2 | 202 | US20150315612 SEQ ID NO: 167 |
| AAVhu.145.6 | 203 | US20150315612 SEQ ID No: 178 |
| AAVhu.15 | 204 | US20150315612 SEQ ID NO: 147 |
| AAVhu.15 (AAV33.4) | 205 | US20150315612 SEQ ID NO: 50 |
| AAVhu.156.1 | 206 | US20150315612 SEQ ID No: 179 |
| AAVhu.16 | 207 | US20150315612 SEQ ID NO: 148 |
| AAVhu.16 (AAV33.8) | 208 | US20150315612 SEQ ID NO: 51 |
| AAVhu.17 | 209 | US20150315612 SEQ ID NO: 83 |
| AAVhu.17 (AAV33.12) | 210 | US20150315612 SEQ ID NO: 4 |
| AAVhu.172.1 | 211 | US20150315612 SEQ ID NO: 171 |
| AAVhu.172.2 | 212 | US20150315612 SEQ ID NO: 172 |
| AAVhu.173.4 | 213 | US20150315612 SEQ ID NO: 173 |
| AAVhu.173.8 | 214 | US20150315612 SEQ ID NO: 175 |
| AAVhu.18 | 215 | US20150315612 SEQ ID NO: 52 |
| AAVhu.18 | 216 | US20150315612 SEQ ID NO: 149 |
| AAVhu.19 | 217 | US20150315612 SEQ ID NO: 62 |
| AAVhu.19 | 218 | US20150315612 SEQ ID NO: 133 |
| AAVhu.2 | 219 | US20150315612 SEQ ID NO: 48 |
| AAVhu.2 | 220 | US20150315612 SEQ ID NO: 143 |
| AAVhu.20 | 221 | US20150315612 SEQ ID NO: 63 |
| AAVhu.20 | 222 | US20150315612 SEQ ID NO: 134 |
| AAVhu.21 | 223 | US20150315612 SEQ ID NO: 65 |
| AAVhu.21 | 224 | US20150315612 SEQ ID NO: 135 |
| AAVhu.22 | 225 | US20150315612 SEQ ID NO: 67 |
| AAVhu.22 | 226 | US20150315612 SEQ ID NO: 138 |
| AAVhu.23 | 227 | US20150315612 SEQ ID NO: 60 |
| AAVhu.23.2 | 228 | US20150315612 SEQ ID NO: 137 |
| AAVhu.24 | 229 | US20150315612 SEQ ID NO: 66 |
| AAVhu.24 | 230 | US20150315612 SEQ ID NO: 136 |
| AAVhu.25 | 231 | US20150315612 SEQ ID NO: 49 |
| AAVhu.25 | 232 | US20150315612 SEQ ID NO: 146 |
| AAVhu.26 | 233 | US20150159173 SEQ ID NO: 17, US20150315612 SEQ ID NO: 61 |
| AAVhu.26 | 234 | US20150159173 SEQ ID NO: 33, US20150315612 SEQ ID NO: 139 |
| AAVhu.27 | 235 | US20150315612 SEQ ID NO: 64 |
| AAVhu.27 | 236 | US20150315612 SEQ ID NO: 140 |
| AAVhu.28 | 237 | US20150315612 SEQ ID NO: 68 |
| AAVhu.28 | 238 | US20150315612 SEQ ID NO: 130 |
| AAVhu.29 | 239 | US20150315612 SEQ ID NO: 69 |
| AAVhu.29 | 240 | US20150159173 SEQ ID NO: 42, US20150315612 SEQ ID NO: 132 |
| AAVhu.29 | 241 | US20150315612 SEQ ID NO: 225 |
| AAVhu.29R | 242 | US20150159173 |
| AAVhu.3 | 243 | US20150315612 SEQ ID NO: 44 |
| AAVhu.3 | 244 | US20150315612 SEQ ID NO: 145 |
| AAVhu.30 | 245 | US20150315612 SEQ ID NO: 70 |
| AAVhu.30 | 246 | US20150315612 SEQ ID NO: 131 |
| AAVhu.31 | 247 | US20150315612 SEQ ID NO: 1 |
| AAVhu.31 | 248 | US20150315612 SEQ ID NO: 121 |
| AAVhu.32 | 249 | US20150315612 SEQ ID NO: 2 |
| AAVhu.32 | 250 | US20150315612 SEQ ID NO: 122 |
| AAVhu.33 | 251 | US20150315612 SEQ ID NO: 75 |
| AAVhu.33 | 252 | US20150315612 SEQ ID NO: 124 |
| AAVhu.34 | 253 | US20150315612 SEQ ID NO: 72 |
| AAVhu.34 | 254 | US20150315612 SEQ ID NO: 125 |
| AAVhu.35 | 255 | US20150315612 SEQ ID NO: 73 |
| AAVhu.35 | 256 | US20150315612 SEQ ID NO: 164 |
| AAVhu.36 | 257 | US20150315612 SEQ ID NO: 74 |
| AAVhu.36 | 258 | US20150315612 SEQ ID NO: 126 |
| AAVhu.37 | 259 | US20150159173 SEQ ID NO: 34, US20150315612 SEQ ID NO: 88 |
| AAVhu.37 (AAV106.1) | 260 | US20150315612 SEQ ID NO: 10, US20150159173 SEQ ID NO: 18 |
| AAVhu.38 | 261 | US20150315612 SEQ ID NO: 161 |
| AAVhu.39 | 262 | US20150315612 SEQ ID NO: 102 |
| AAVhu.39 (AAVLG-9) | 263 | US20150315612 SEQ ID NO: 24 |
| AAVhu.4 | 264 | US20150315612 SEQ ID NO: 47 |
| AAVhu.4 | 265 | US20150315612 SEQ ID NO: 141 |
| AAVhu.40 | 266 | US20150315612 SEQ ID NO: 87 |
| AAVhu.40 (AAV114.3) | 267 | US20150315612 SEQ ID No. 11 |
| AAVhu.41 | 268 | US20150315612 SEQ ID NO: 91 |
| AAVhu.41 (AAV127.2) | 269 | US20150315612 SEQ ID NO: 6 |
| AAVhu.42 | 270 | US20150315612 SEQ ID NO: 85 |
| AAVhu.42 (AAV127.5) | 271 | US20150315612 SEQ ID NO: 8 |
| AAVhu.43 | 272 | US20150315612 SEQ ID NO: 160 |
| AAVhu.43 | 273 | US20150315612 SEQ ID NO: 236 |
| AAVhu.43 (AAV128.1) | 274 | US20150315612 SEQ ID NO: 80 |
| AAVhu.44 | 275 | US20150159173 SEQ ID NO: 45, US20150315612 SEQ ID NO: 158 |
| AAVhu.44 (AAV128.3) | 276 | US20150315612 SEQ ID NO: 81 |
| AAVhu.44R1 | 277 | US20150159173 |
| AAVhu.44R2 | 278 | US20150159173 |
| AAVhu.44R3 | 279 | US20150159173 |
| AAVhu.45 | 280 | US20150315612 SEQ ID NO: 76 |
| AAVhu.45 | 281 | US20150315612 SEQ ID NO: 127 |
| AAVhu.46 | 282 | US20150315612 SEQ ID NO: 82 |
| AAVhu.46 | 283 | US20150315612 SEQ ID NO: 159 |
| AAVhu.46 | 284 | US20150315612 SEQ ID NO: 224 |
| AAVhu.47 | 285 | US20150315612 SEQ ID NO: 77 |
| AAVhu.47 | 286 | US20150315612 SEQ ID NO: 128 |
| AAVhu.48 | 287 | US20150159173 SEQ ID NO: 38 |
| AAVhu.48 | 288 | US20150315612 SEQ ID NO: 157 |
| AAVhu.48 (AAV130.4) | 289 | US20150315612 SEQ ID NO: 78 |
| AAVhu.48R1 | 290 | US20150159173 |
| AAVhu.48R2 | 291 | US20150159173 |
| AAVhu.48R3 | 292 | US20150159173 |
| AAVhu.49 | 293 | US20150315612 SEQ ID NO: 209 |
| AAVhu.49 | 294 | US20150315612 SEQ ID NO: 189 |
| AAVhu.5 | 295 | US20150315612 SEQ ID NO: 45 |
| AAVhu.5 | 296 | US20150315612 SEQ ID NO: 142 |
| AAVhu.51 | 297 | US20150315612 SEQ ID NO: 208 |
| AAVhu.51 | 298 | US20150315612 SEQ ID NO: 190 |
| AAVhu.52 | 299 | US20150315612 SEQ ID NO: 210 |
| AAVhu.52 | 300 | US20150315612 SEQ ID NO: 191 |
| AAVhu.53 | 301 | US20150159173 SEQ ID NO: 19 |
| AAVhu.53 | 302 | US20150159173 SEQ ID NO: 35 |
| AAVhu.53 (AAV145.1) | 303 | US20150315612 SEQ ID NO: 176 |
| AAVhu.54 | 304 | US20150315612 SEQ ID NO: 188 |
| AAVhu.54 (AAV145.5) | 305 | US20150315612 SEQ ID No: 177 |
| AAVhu.55 | 306 | US20150315612 SEQ ID NO: 187 |
| AAVhu.56 | 307 | US20150315612 SEQ ID NO: 205 |
| AAVhu.56 (AAV145.6) | 308 | US20150315612 SEQ ID NO: 168 |
| AAVhu.56 (AAV145.6) | 309 | US20150315612 SEQ ID NO: 192 |
| AAVhu.57 | 310 | US20150315612 SEQ ID NO: 206 |
| AAVhu.57 | 311 | US20150315612 SEQ ID NO: 169 |
| AAVhu.57 | 312 | US20150315612 SEQ ID NO: 193 |
| AAVhu.58 | 313 | US20150315612 SEQ ID NO: 207 |
| AAVhu.58 | 314 | US20150315612 SEQ ID NO: 194 |
| AAVhu.6 (AAV3.1) | 315 | US20150315612 SEQ ID NO: 5 |
| AAVhu.6 (AAV3.1) | 316 | US20150315612 SEQ ID NO: 84 |
| AAVhu.60 | 317 | US20150315612 SEQ ID NO: 184 |
| AAVhu.60 (AAV161.10) | 318 | US20150315612 SEQ ID NO: 170 |
| AAVhu.61 | 319 | US20150315612 SEQ ID NO: 185 |
| AAVhu.61 (AAV161.6) | 320 | US20150315612 SEQ ID NO: 174 |
| AAVhu.63 | 321 | US20150315612 SEQ ID NO: 204 |
| AAVhu.63 | 322 | US20150315612 SEQ ID NO: 195 |
| AAVhu.64 | 323 | US20150315612 SEQ ID NO: 212 |
| AAVhu.64 | 324 | US20150315612 SEQ ID NO: 196 |
| AAVhu.66 | 325 | US20150315612 SEQ ID NO: 197 |
| AAVhu.67 | 326 | US20150315612 SEQ ID NO: 215 |
| AAVhu.67 | 327 | US20150315612 SEQ ID NO: 198 |
| AAVhu.7 | 328 | US20150315612 SEQ ID NO: 226 |
| AAVhu.7 | 329 | US20150315612 SEQ ID NO: 150 |
| AAVhu.7 (AAV7.3) | 330 | US20150315612 SEQ ID NO: 55 |
| AAVhu.71 | 331 | US20150315612 SEQ ID NO: 79 |
| AAVhu.8 | 332 | US20150315612 SEQ ID NO: 53 |
| AAVhu.8 | 333 | US20150315612 SEQ ID NO: 12 |
| AAVhu.8 | 334 | US20150315612 SEQ ID NO: 151 |
| AAVhu.9 (AAV3.1) | 335 | US20150315612 SEQ ID NO: 58 |
| AAVhu.9 (AAV3.1) | 336 | US20150315612 SEQ ID NO: 155 |
| AAV-LK01 | 337 | US20150376607 SEQ ID NO: 2 |
| AAV-LK01 | 338 | US20150376607 SEQ ID NO: 29 |
| AAV-LK02 | 339 | US20150376607 SEQ ID NO: 3 |
| AAV-LK02 | 340 | US20150376607 SEQ ID NO: 30 |
| AAV-LK03 | 341 | US20150376607 SEQ ID NO: 4 |
| AAV-LK03 | 342 | WO2015121501 SEQ ID NO: 12, US20150376607 SEQ ID NO: 31 |
| AAV-LK04 | 343 | US20150376607 SEQ ID NO: 5 |
| AAV-LK04 | 344 | US20150376607 SEQ ID NO: 32 |
| AAV-LK05 | 345 | US20150376607 SEQ ID NO: 6 |
| AAV-LK05 | 346 | US20150376607 SEQ ID NO: 33 |
| AAV-LK06 | 347 | US20150376607 SEQ ID NO: 7 |
| AAV-LK06 | 348 | US20150376607 SEQ ID NO: 34 |
| AAV-LK07 | 349 | US20150376607 SEQ ID NO: 8 |
| AAV-LK07 | 350 | US20150376607 SEQ ID NO: 35 |
| AAV-LK08 | 351 | US20150376607 SEQ ID NO: 9 |
| AAV-LK08 | 352 | US20150376607 SEQ ID NO: 36 |
| AAV-LK09 | 353 | US20150376607 SEQ ID NO: 10 |
| AAV-LK09 | 354 | US20150376607 SEQ ID NO: 37 |
| AAV-LK10 | 355 | US20150376607 SEQ ID NO: 11 |
| AAV-LK10 | 356 | US20150376607 SEQ ID NO: 38 |
| AAV-LK11 | 357 | US20150376607 SEQ ID NO: 12 |
| AAV-LK11 | 358 | US20150376607 SEQ ID NO: 39 |
| AAV-LK12 | 359 | US20150376607 SEQ ID NO: 13 |
| AAV-LK12 | 360 | US20150376607 SEQ ID NO: 40 |
| AAV-LK13 | 361 | US20150376607 SEQ ID NO: 14 |
| AAV-LK13 | 362 | US20150376607 SEQ ID NO: 41 |
| AAV-LK14 | 363 | US20150376607 SEQ ID NO: 15 |
| AAV-LK14 | 364 | US20150376607 SEQ ID NO: 42 |
| AAV-LK15 | 365 | US20150376607 SEQ ID NO: 16 |
| AAV-LK15 | 366 | US20150376607 SEQ ID NO: 43 |
| AAV-LK16 | 367 | US20150376607 SEQ ID NO: 17 |
| AAV-LK16 | 368 | US20150376607 SEQ ID NO: 44 |
| AAV-LK17 | 369 | US20150376607 SEQ ID NO: 18 |
| AAV-LK17 | 370 | US20150376607 SEQ ID NO: 45 |
| AAV-LK18 | 371 | US20150376607 SEQ ID NO: 19 |
| AAV-LK18 | 372 | US20150376607 SEQ ID NO: 46 |
| AAV-LK19 | 373 | US20150376607 SEQ ID NO: 20 |
| AAV-LK19 | 374 | US20150376607 SEQ ID NO: 47 |
| AAV-PAEC | 375 | US20150376607 SEQ ID NO: 1 |
| AAV-PAEC | 376 | US20150376607 SEQ ID NO: 48 |
| AAV-PAEC11 | 377 | US20150376607 SEQ ID NO: 26 |
| AAV-PAEC11 | 378 | US20150376607 SEQ ID NO: 54 |
| AAV-PAEC12 | 379 | US20150376607 SEQ ID NO: 27 |
| AAV-PAEC12 | 380 | US20150376607 SEQ ID NO: 51 |
| AAV-PAEC13 | 381 | US20150376607 SEQ ID NO: 28 |
| AAV-PAEC13 | 382 | US20150376607 SEQ ID NO: 49 |
| AAV-PAEC2 | 383 | US20150376607 SEQ ID NO: 21 |
| AAV-PAEC2 | 384 | US20150376607 SEQ ID NO: 56 |
| AAV-PAEC4 | 385 | US20150376607 SEQ ID NO: 22 |
| AAV-PAEC4 | 386 | US20150376607 SEQ ID NO: 55 |
| AAV-PAEC6 | 387 | US20150376607 SEQ ID NO: 23 |
| AAV-PAEC6 | 388 | US20150376607 SEQ ID NO: 52 |
| AAV-PAEC7 | 389 | US20150376607 SEQ ID NO: 24 |
| AAV-PAEC7 | 390 | US20150376607 SEQ ID NO: 53 |
| AAV-PAEC8 | 391 | US20150376607 SEQ ID NO: 25 |
| AAV-PAEC8 | 392 | US20150376607 SEQ ID NO: 50 |
| AAVpi.1 | 393 | US20150315612 SEQ ID NO: 28 |
| AAVpi.1 | 394 | US20150315612 SEQ ID NO: 93 |
| AAVpi.2 | 395 | US20150315612 SEQ ID NO: 30 |
| AAVpi.2 | 396 | US20150315612 SEQ ID NO: 95 |
| AAVpi.3 | 397 | US20150315612 SEQ ID NO: 29 |
| AAVpi.3 | 398 | US20150315612 SEQ ID NO: 94 |
| AAVrh.10 | 399 | US20150159173 SEQ ID NO: 9 |
| AAVrh.10 | 400 | US20150159173 SEQ ID NO: 25 |
| AAV44.2 | 401 | US20030138772 SEQ ID NO: 59 |
| AAVrh.10 (AAV44.2) | 402 | US20030138772 SEQ ID NO: 81 |
| AAV42.1B | 403 | US20030138772 SEQ ID NO: 90 |
| AAVrh.12 (AAV42.1b) | 404 | US20030138772 SEQ ID NO: 30 |
| AAVrh.13 | 405 | US20150159173 SEQ ID NO: 10 |
| AAVrh.13 | 406 | US20150159173 SEQ ID NO: 26 |
| AAVrh.13 | 407 | US20150315612 SEQ ID NO: 228 |
| AAVrh.13R | 408 | US20150159173 |
| AAV42.3A | 409 | US20030138772 SEQ ID NO: 87 |
| AAVrh.14 (AAV42.3a) | 410 | US20030138772 SEQ ID NO: 32 |
| AAV42.5A | 411 | US20030138772 SEQ ID NO: 89 |
| AAVrh.17 (AAV42.5a) | 412 | US20030138772 SEQ ID NO: 34 |
| AAV42.5B | 413 | US20030138772 SEQ ID NO: 91 |
| AAVrh.18 (AAV42.5b) | 414 | US20030138772 SEQ ID NO: 29 |
| AAV42.6B | 415 | US20030138772 SEQ ID NO: 112 |
| AAVrh.19 (AAV42.6b) | 416 | US20030138772 SEQ ID NO: 38 |
| AAVrh.2 | 417 | US20150159173 SEQ ID NO: 39 |
| AAVrh.2 | 418 | US20150315612 SEQ ID NO: 231 |
| AAVrh.20 | 419 | US20150159173 SEQ ID NO: 1 |
| AAV42.10 | 420 | US20030138772 SEQ ID NO: 106 |
| AAVrh.21 (AAV42.10) | 421 | US20030138772 SEQ ID NO: 35 |
| AAV42.11 | 422 | US20030138772 SEQ ID NO: 108 |
| AAVrh.22 (AAV42.11) | 423 | US20030138772 SEQ ID NO: 37 |
| AAV42.12 | 424 | US20030138772 SEQ ID NO: 113 |
| AAVrh.23 (AAV42.12) | 425 | US20030138772 SEQ ID NO: 58 |
| AAV42.13 | 426 | US20030138772 SEQ ID NO: 86 |
| AAVrh.24 (AAV42.13) | 427 | US20030138772 SEQ ID NO: 31 |
| AAV42.15 | 428 | US20030138772 SEQ ID NO: 84 |
| AAVrh.25 (AAV42.15) | 429 | US20030138772 SEQ ID NO: 28 |
| AAVrh.2R | 430 | US20150159173 |
| AAVrh.31 (AAV223.1) | 431 | US20030138772 SEQ ID NO: 48 |
| AAVC1 | 432 | US20030138772 SEQ ID NO: 60 |
| AAVrh.32 (AAVC1) | 433 | US20030138772 SEQ ID NO: 19 |
| AAVrh.32/33 | 434 | US20150159173 SEQ ID NO: 2 |
| AAVrh.33 (AAVC3) | 435 | US20030138772 SEQ ID NO: 20 |
| AAVC5 | 436 | US20030138772 SEQ ID NO: 62 |
| AAVrh.34 (AAVC5) | 437 | US20030138772 SEQ ID NO: 21 |
| AAVF1 | 438 | US20030138772 SEQ ID NO: 109 |
| AAVrh.35 (AAVF1) | 439 | US20030138772 SEQ ID NO: 22 |
| AAVF3 | 440 | US20030138772 SEQ ID NO: 111 |
| AAVrh.36 (AAVF3) | 441 | US20030138772 SEQ ID NO: 23 |
| AAVrh.37 | 442 | US20030138772 SEQ ID NO: 24 |
| AAVrh.37 | 443 | US20150159173 SEQ ID NO: 40 |
| AAVrh.37 | 444 | US20150315612 SEQ ID NO: 229 |
| AAVrh.37R2 | 445 | US20150159173 |
| AAVrh.38 (AAVLG-4) | 446 | US20150315612 SEQ ID NO: 7 |
| AAVrh.38 (AAVLG-4) | 447 | US20150315612 SEQ ID NO: 86 |
| AAVrh.39 | 448 | US20150159173 SEQ ID NO: 20, US20150315612 SEQ ID NO: 13 |
| AAVrh.39 | 449 | US20150159173 SEQ ID NO: 3, US20150159173 SEQ ID NO: 36, |
| | | US20150315612 SEQ ID NO: 89 |
| AAVrh.40 | 450 | US20150315612 SEQ ID NO: 92 |
| AAVrh.40 (AAVLG-10) | 451 | US20150315612 SEQ ID No: 14 |
| AAVrh.43 (AAVN721-8) | 452 | US20150315612 SEQ ID NO: 43, US20150159173 SEQ ID NO: 21 |
| AAVrh.43 (AAVN721-8) | 453 | US20150315612 SEQ ID NO: 163, US20150159173 SEQ ID NO: 37 |
| AAVrh.44 | 454 | US20150315612 SEQ ID NO: 34 |
| AAVrh.44 | 455 | US20150315612 SEQ ID NO: 111 |
| AAVrh.45 | 456 | US20150315612 SEQ ID NO: 41 |
| AAVrh.45 | 457 | US20150315612 SEQ ID NO: 109 |
| AAVrh.46 | 458 | US20150159173 SEQ ID NO: 22, US20150315612 SEQ ID NO: 19 |
| AAVrh.46 | 459 | US20150159173 SEQ ID NO: 4, US20150315612 SEQ ID NO: 101 |
| AAVrh.47 | 460 | US20150315612 SEQ ID NO: 38 |
| AAVrh.47 | 461 | US20150315612 SEQ ID NO: 118 |
| AAVrh.48 | 462 | US20150159173 SEQ ID NO: 44, US20150315612 SEQ ID NO: 115 |
| AAVrh.48.1 | 463 | US20150159173 |
| AAVrh.48.1.2 | 464 | US20150159173 |
| AAVrh.48.2 | 465 | US20150159173 |
| AAVrh.48 (AAV1-7) | 466 | US20150315612 SEQ ID NO: 32 |
| AAVrh.49 (AAV1-8) | 467 | US20150315612 SEQ ID NO: 25 |
| AAVrh.49 (AAV1-8) | 468 | US20150315612 SEQ ID NO: 103 |
| AAVrh.50 (AAV2-4) | 469 | US20150315612 SEQ ID NO: 23 |
| AAVrh.50 (AAV2-4) | 470 | US20150315612 SEQ ID NO: 108 |
| AAVrh.51 (AAV2-5) | 471 | US20150315612 SEQ ID No: 22 |
| AAVrh.51 (AAV2-5) | 472 | US20150315612 SEQ ID NO: 104 |
| AAVrh.52 (AAV3-9) | 473 | US20150315612 SEQ ID NO: 18 |
| AAVrh.52 (AAV3-9) | 474 | US20150315612 SEQ ID NO: 96 |
| AAVrh.53 | 475 | US20150315612 SEQ ID NO: 97 |
| AAVrh.53 (AAV3-11) | 476 | US20150315612 SEQ ID NO: 17 |
| AAVrh.53 (AAV3-11) | 477 | US20150315612 SEQ ID NO: 186 |
| AAVrh.54 | 478 | US20150315612 SEQ ID NO: 40 |
| AAVrh.54 | 479 | US20150159173 SEQ ID NO: 49, US20150315612 SEQ ID NO: 116 |
| AAVrh.55 | 480 | US20150315612 SEQ ID NO: 37 |
| AAVrh.55 (AAV4-19) | 481 | US20150315612 SEQ ID NO: 117 |
| AAVrh.56 | 482 | US20150315612 SEQ ID NO: 54 |
| AAVrh.56 | 483 | US20150315612 SEQ ID NO: 152 |
| AAVrh.57 | 484 | US20150315612 SEQ ID NO: 26 |
| AAVrh.57 | 485 | US20150315612 SEQ ID NO: 105 |
| AAVrh.58 | 486 | US20150315612 SEQ ID NO: 27 |
| AAVrh.58 | 487 | US20150159173 SEQ ID NO: 48, US20150315612 SEQ ID NO: 106 |
| AAVrh.58 | 488 | US20150315612 SEQ ID NO: 232 |
| AAVrh.59 | 489 | US20150315612 SEQ ID NO: 42 |
| AAVrh.59 | 490 | US20150315612 SEQ ID NO: 110 |
| AAVrh.60 | 491 | US20150315612 SEQ ID NO: 31 |
| AAVrh.60 | 492 | US20150315612 SEQ ID NO: 120 |
| AAVrh.61 | 493 | US20150315612 SEQ ID NO: 107 |
| AAVrh.61 (AAV2-3) | 494 | US20150315612 SEQ ID NO: 21 |
| AAVrh.62 (AAV2-15) | 495 | US20150315612 SEQ ID No: 33 |
| AAVrh.62 (AAV2-15) | 496 | US20150315612 SEQ ID NO: 114 |
| AAVrh.64 | 497 | US20150315612 SEQ ID No: 15 |
| AAVrh.64 | 498 | US20150159173 SEQ ID NO: 43, US20150315612 SEQ ID NO: 99 |
| AAVrh.64 | 499 | US20150315612 SEQ ID NO: 233 |
| AAVRh.64R1 | 500 | US20150159173 |
| AAVRh.64R2 | 501 | US20150159173 |
| AAVrh.65 | 502 | US20150315612 SEQ ID NO: 35 |
| AAVrh.65 | 503 | US20150315612 SEQ ID NO: 112 |
| AAVrh.67 | 504 | US20150315612 SEQ ID NO: 36 |
| AAVrh.67 | 505 | US20150315612 SEQ ID NO: 230 |
| AAVrh.67 | 506 | US20150159173 SEQ ID NO: 47, US20150315612 SEQ ID NO: 113 |
| AAVrh.68 | 507 | US20150315612 SEQ ID NO: 16 |
| AAVrh.68 | 508 | US20150315612 SEQ ID NO: 100 |
| AAVrh.69 | 509 | US20150315612 SEQ ID NO: 39 |
| AAVrh.69 | 510 | US20150315612 SEQ ID NO: 119 |
| AAVrh.70 | 511 | US20150315612 SEQ ID NO: 20 |
| AAVrh.70 | 512 | US20150315612 SEQ ID NO: 98 |
| AAVrh.71 | 513 | US20150315612 SEQ ID NO: 162 |
| AAVrh.72 | 514 | US20150315612 SEQ ID NO: 9 |
| AAVrh.73 | 515 | US20150159173 SEQ ID NO: 5 |
| AAVrh.74 | 516 | US20150159173 SEQ ID NO: 6 |
| AAVrh.8 | 517 | US20150159173 SEQ ID NO: 41 |
| AAVrh.8 | 518 | US20150315612 SEQ ID NO: 235 |
| AAVrh.8R | 519 | US20150159173, WO2015168666 SEQ ID NO: 9 |
| AAVrh.8R A586R mutant | 520 | WO2015168666 SEQ ID NO: 10 |
| AAVrh.8R R533A mutant | 521 | WO2015168666 SEQ ID NO: 11 |
| BAAV (bovine AAV) | 522 | U.S. Pat. No. 9,193,769 SEQ ID NO: 8 |
| BAAV (bovine AAV) | 523 | U.S. Pat. No. 9,193,769 SEQ ID NO: 10 |
| BAAV (bovine AAA) | 524 | U.S. Pat. No. 9,193,769 SEQ ID NO: 4 |
| BAAV (bovine AAV) | 525 | U.S. Pat. No. 9,193,769 SEQ ID NO: 2 |
| BAAV (bovine AAV) | 526 | U.S. Pat. No. 9,193,769 SEQ ID NO: 6 |
| BAAV (bovine AAV) | 527 | U.S. Pat. No. 9,193,769 SEQ ID NO: 1 |
| BAAV (bovine AAV) | 528 | U.S. Pat. No. 9,193,769 SEQ ID NO: 5 |
| BAAV (bovine AAV) | 529 | U.S. Pat. No. 9,193,769 SEQ ID NO: 3 |
| BAAV (bovine AAV) | 530 | U.S. Pat. No. 9,193,769 SEQ ID NO: 11 |
| BAAV (bovine AAA) | 531 | U.S. Pat. No. 7,427,396 SEQ ID NO: 5 |
| BAAV (bovine AAV) | 532 | U.S. Pat. No. 7,427,396 SEQ ID NO: 6 |
| BAAV (bovine AAV) | 533 | U.S. Pat. No. 9,193,769 SEQ ID NO: 7 |
| BAAV (bovine AAV) | 534 | U.S. Pat. No. 9,193,769 SEQ ID NO: 9 |
| BNP61 AAV | 535 | US20150238550 SEQ ID NO: 1 |
| BNP61 AAV | 536 | US20150238550 SEQ ID NO: 2 |
| BNP62 AAV | 537 | US20150238550 SEQ ID NO: 3 |
| BNP63 AAV | 538 | US20150238550 SEQ ID NO: 4 |
| caprine AAV | 539 | U.S. Pat. No. 7,427,396 SEQ ID NO: 3 |
| caprine AAV | 540 | U.S. Pat. No. 7,427,396 SEQ ID NO: 4 |
| true type AAV (ttAAV) | 541 | WO2015121501 SEQ ID NO: 2 |
| AAAV (Avian AAV) | 542 | U.S. Pat. No. 9,238,800 SEQ ID NO: 12 |
| AAAV (Avian AAV) | 543 | U.S. Pat. No. 9,238,800 SEQ ID NO: 2 |
| AAAV (Avian AAV) | 544 | U.S. Pat. No. 9,238,800 SEQ ID NO: 6 |
| AAAV (Avian AAV) | 545 | U.S. Pat. No. 9,238,800 SEQ ID NO: 4 |
| AAAV (Avian AAV) | 546 | U.S. Pat. No. 9,238,800 SEQ ID NO: 8 |
| AAAV (Avian AAV) | 547 | U.S. Pat. No. 9,238,800 SEQ ID NO: 14 |
| AAAV (Avian AAV) | 548 | U.S. Pat. No. 9,238,800 SEQ ID NO: 10 |
| AAAV (Avian AAV) | 549 | U.S. Pat. No. 9,238,800 SEQ ID NO: 15 |
| AAAV (Avian AAV) | 550 | U.S. Pat. No. 9,238,800 SEQ ID NO: 5 |
| AAAV (Avian AAV) | 551 | U.S. Pat. No. 9,238,800 SEQ ID NO: 9 |
| AAAV (Avian AAV) | 552 | U.S. Pat. No. 9,238,800 SEQ ID NO: 3 |
| AAAV (Avian AAV) | 553 | U.S. Pat. No. 9,238,800 SEQ ID NO: 7 |
| AAAV (Avian AAV) | 554 | U.S. Pat. No. 9,238,800 SEQ ID NO: 11 |
| AAAV (Avian AAV) | 555 | U.S. Pat. No. 9,238,800 SEQ ID NO: 13 |
| AAAV (Avian AAV) | 556 | U.S. Pat. No. 9,238,800 SEQ ID NO: 1 |
| AAV Shuffle 100-1 | 557 | US20160017295 SEQ ID NO: 23 |
| AAV Shuffle 100-1 | 558 | US20160017295 SEQ ID NO: 11 |
| AAV Shuffle 100-2 | 559 | US20160017295 SEQ ID NO: 37 |
| AAV Shuffle 100-2 | 560 | US20160017295 SEQ ID NO: 29 |
| AAV Shuffle 100-3 | 561 | US20160017295 SEQ ID NO: 24 |
| AAV Shuffle 100-3 | 562 | US20160017295 SEQ ID NO: 12 |
| AAV Shuffle 100-7 | 563 | US20160017295 SEQ ID NO: 25 |
| AAV Shuffle 100-7 | 564 | US20160017295 SEQ ID NO: 13 |
| AAV Shuffle 10-2 | 565 | US20160017295 SEQ ID NO: 34 |
| AAV Shuffle 10-2 | 566 | US20160017295 SEQ ID NO: 26 |
| AAV Shuffle 10-6 | 567 | US20160017295 SEQ ID NO: 35 |
| AAV Shuffle 10-6 | 568 | US20160017295 SEQ ID NO: 27 |
| AAV Shuffle 10-8 | 569 | US20160017295 SEQ ID NO: 36 |
| AAV Shuffle 10-8 | 570 | US20160017295 SEQ ID NO: 28 |
| AAV SM 100-10 | 571 | US20160017295 SEQ ID NO: 41 |
| AAV SM 100-10 | 572 | US20160017295 SEQ ID NO: 33 |
| AAV SM 100-3 | 573 | US20160017295 SEQ ID NO: 40 |
| AAV SM 100-3 | 574 | US20160017295 SEQ ID NO: 32 |
| AAV SM 10-1 | 575 | US20160017295 SEQ ID NO: 38 |
| AAV SM 10-1 | 576 | US20160017295 SEQ ID NO: 30 |
| AAV SM 10-2 | 577 | US20160017295 SEQ ID NO: 10 |
| AAV SM 10-2 | 578 | US20160017295 SEQ ID NO: 22 |
| AAV SM 10-8 | 579 | US20160017295 SEQ ID NO: 39 |
| AAV SM 10-8 | 580 | US20160017295 SEQ ID NO: 31 |
| AAVF1/HSC1 | 581 | WO2016049230 SEQ ID NO: 20 |
| AAVF2/HSC2 | 582 | WO2016049230 SEQ ID NO: 21 |
| AAVF3/HSC3 | 583 | WO2016049230 SEQ ID NO: 22 |
| AAVF4/HSC4 | 584 | WO2016049230 SEQ ID NO: 23 |
| AAVF5/HSC5 | 585 | WO2016049230 SEQ ID NO: 25 |
| AAVF6/HSC6 | 586 | WO2016049230 SEQ ID NO: 24 |
| AAVF7/HSC7 | 587 | WO2016049230 SEQ ID NO: 27 |
| AAVF8/HSC8 | 588 | WO2016049230 SEQ ID NO: 28 |
| AAVF9/HSC9 | 589 | WO2016049230 SEQ ID NO: 29 |
| AAVF11/HSC11 | 590 | WO2016049230 SEQ ID NO: 26 |
| AAVF12/HSC12 | 591 | WO2016049230 SEQ ID NO: 30 |
| AAVF13/HSC13 | 592 | WO2016049230 SEQ ID NO: 31 |
| AAVF14/HSC14 | 593 | WO2016049230 SEQ ID NO: 32 |
| AAVF15/HSC15 | 594 | WO2016049230 SEQ ID NO: 33 |
| AAVF16/HSC16 | 595 | WO2016049230 SEQ ID NO: 34 |
| AAVF17/HSC17 | 596 | WO2016049230 SEQ ID NO: 35 |
| AAVF1/HSC1 | 597 | WO2016049230 SEQ ID NO: 2 |
| AAVF2/HSC2 | 598 | WO2016049230 SEQ ID NO: 3 |
| AAVF3/HSC3 | 599 | WO2016049230 SEQ ID NO: 5 |
| AAVF4/HSC4 | 600 | WO2016049230 SEQ ID NO: 6 |
| AAVF5/HSC5 | 601 | WO2016049230 SEQ ID NO: 11 |
| AAVF6/HSC6 | 602 | WO2016049230 SEQ ID NO: 7 |
| AAVF7/HSC7 | 603 | WO2016049230 SEQ ID NO: 8 |
| AAVF8/HSC8 | 604 | WO2016049230 SEQ ID NO: 9 |
| AAVF9/HSC9 | 605 | WO2016049230 SEQ ID NO: 10 |
| AAVF11/HSC11 | 606 | WO2016049230 SEQ ID NO: 4 |
| AAVF12/HSC12 | 607 | WO2016049230 SEQ ID NO: 12 |
| AAVF13/HSC13 | 608 | WO2016049230 SEQ ID NO: 14 |
| AAVF14/HSC14 | 609 | WO2016049230 SEQ ID NO: 15 |
| AAVF15/HSC15 | 610 | WO2016049230 SEQ ID NO: 16 |
| AAVF16/HSC16 | 611 | WO2016049230 SEQ ID NO: 17 |
| AAVF17/HSC17 | 612 | WO2016049230 SEQ ID NO: 13 |
| AAV CBr-E1 | 613 | U.S. Pat. No. 8,734,809 SEQ ID NO: 13 |
| AAV CBr-E2 | 614 | U.S. Pat. No. 8,734,809 SEQ ID NO: 14 |
| AAV CBr-E3 | 615 | U.S. Pat. No. 8,734,809 SEQ ID NO: 15 |
| AAV CBr-E4 | 616 | U.S. Pat. No. 8,734,809 SEQ ID NO: 16 |
| AAV CBr-E5 | 617 | U.S. Pat. No. 8,734,809 SEQ ID NO: 17 |
| AAV CBr-e5 | 618 | U.S. Pat. No. 8,734,809 SEQ ID NO: 18 |
| AAV CBr-E6 | 619 | U.S. Pat. No. 8,734,809 SEQ ID NO: 19 |
| AAV CBr-E7 | 620 | U.S. Pat. No. 8,734,809 SEQ ID NO: 20 |
| AAV CBr-E8 | 621 | U.S. Pat. No. 8,734,809 SEQ ID NO: 21 |
| AAV CLv-D1 | 622 | U.S. Pat. No. 8,734,809 SEQ ID NO: 22 |
| AAV CLv-D2 | 623 | U.S. Pat. No. 8,734,809 SEQ ID NO: 23 |
| AAV CLv-D3 | 624 | U.S. Pat. No. 8,734,809 SEQ ID NO: 24 |
| AAV CLv-D4 | 625 | U.S. Pat. No. 8,734,809 SEQ ID NO: 25 |
| AAV CLv-D5 | 626 | U.S. Pat. No. 8,734,809 SEQ ID NO: 26 |
| AAV CLv-D6 | 627 | U.S. Pat. No. 8,734,809 SEQ ID NO: 27 |
| AAV CLv-D7 | 628 | U.S. Pat. No. 8,734,809 SEQ ID NO: 28 |
| AAV CLv-D8 | 629 | U.S. Pat. No. 8,734,809 SEQ ID NO: 29 |
| AAV CLv-E1 | 630 | U.S. Pat. No. 8,734,809 SEQ ID NO: 13 |
| AAV CLv-R1 | 631 | U.S. Pat. No. 8,734,809 SEQ ID NO: 30 |
| AAV CLv-R2 | 632 | U.S. Pat. No. 8,734,809 SEQ ID NO: 31 |
| AAV CLv-R3 | 633 | U.S. Pat. No. 8,734,809 SEQ ID NO: 32 |
| AAV CLv-R4 | 634 | U.S. Pat. No. 8,734,809 SEQ ID NO: 33 |
| AAV CLv-R5 | 635 | U.S. Pat. No. 8,734,809 SEQ ID NO: 34 |
| AAV CLv-R6 | 636 | U.S. Pat. No. 8,734,809 SEQ ID NO: 35 |
| AAV CLv-R7 | 637 | U.S. Pat. No. 8,734,809 SEQ ID NO: 36 |
| AAV CLv-R8 | 638 | U.S. Pat. No. 8,734,809 SEQ ID NO: 37 |
| AAV CLv-R9 | 639 | U.S. Pat. No. 8,734,809 SEQ ID NO: 38 |
| AAV CLg-F1 | 640 | U.S. Pat. No. 8,734,809 SEQ ID NO: 39 |
| AAV CLg-F2 | 641 | U.S. Pat. No. 8,734,809 SEQ ID NO: 40 |
| AAV CLg-F3 | 642 | U.S. Pat. No. 8,734,809 SEQ ID NO: 41 |
| AAV CLg-F4 | 643 | U.S. Pat. No. 8,734,809 SEQ ID NO: 42 |
| AAV CLg-F5 | 644 | U.S. Pat. No. 8,734,809 SEQ ID NO: 43 |
| AAV CLg-F6 | 645 | U.S. Pat. No. 8,734,809 SEQ ID NO: 43 |
| AAV CLg-F7 | 646 | U.S. Pat. No. 8,734,809 SEQ ID NO: 44 |
| AAV CLg-F8 | 647 | U.S. Pat. No. 8,734,809 SEQ ID NO: 43 |
| AAV CSp-1 | 648 | U.S. Pat. No. 8,734,809 SEQ ID NO: 45 |
| AAV CSp-10 | 649 | U.S. Pat. No. 8,734,809 SEQ ID NO: 46 |
| AAV CSp-11 | 650 | U.S. Pat. No. 8,734,809 SEQ ID NO: 47 |
| AAV CSp-2 | 651 | U.S. Pat. No. 8,734,809 SEQ ID NO: 48 |
| AAV CSp-3 | 652 | U.S. Pat. No. 8,734,809 SEQ ID NO: 49 |
| AAV CSp-4 | 653 | U.S. Pat. No. 8,734,809 SEQ ID NO: 50 |
| AAV CSp-6 | 654 | U.S. Pat. No. 8,734,809 SEQ ID NO: 51 |
| AAV CSp-7 | 655 | U.S. Pat. No. 8,734,809 SEQ ID NO: 52 |
| AAV CSp-8 | 656 | U.S. Pat. No. 8,734,809 SEQ ID NO: 53 |
| AAV CSp-9 | 657 | U.S. Pat. No. 8,734,809 SEQ ID NO: 54 |
| AAV CHt-2 | 658 | U.S. Pat. No. 8,734,809 SEQ ID NO: 55 |
| AAV CHt-3 | 659 | U.S. Pat. No. 8,734,809 SEQ ID NO: 56 |
| AAV CKd-1 | 660 | U.S. Pat. No. 8,734,809 SEQ ID NO: 57 |
| AAV CKd-10 | 661 | U.S. Pat. No. 8,734,809 SEQ ID NO: 58 |
| AAV CKd-2 | 662 | U.S. Pat. No. 8,734,809 SEQ ID NO: 59 |
| AAV CKd-3 | 663 | U.S. Pat. No. 8,734,809 SEQ ID NO: 60 |
| AAV CKd-4 | 664 | U.S. Pat. No. 8,734,809 SEQ ID NO: 61 |
| AAV CKd-6 | 665 | U.S. Pat. No. 8,734,809 SEQ ID NO: 62 |
| AAV CKd-7 | 666 | U.S. Pat. No. 8,734,809 SEQ ID NO: 63 |
| AAV CKd-8 | 667 | U.S. Pat. No. 8,734,809 SEQ ID NO: 64 |
| AAV CLv-1 | 668 | U.S. Pat. No. 8,734,809 SEQ ID NO: 65 |
| AAV CLv-12 | 669 | U.S. Pat. No. 8,734,809 SEQ ID NO: 66 |
| AAV CLv-13 | 670 | U.S. Pat. No. 8,734,809 SEQ ID NO: 67 |
| AAV CLv-2 | 671 | U.S. Pat. No. 8,734,809 SEQ ID NO: 68 |
| AAV CLv-3 | 672 | U.S. Pat. No. 8,734,809 SEQ ID NO: 69 |
| AAV CLv-4 | 673 | U.S. Pat. No. 8,734,809 SEQ ID NO: 70 |
| AAV CLv-6 | 674 | U.S. Pat. No. 8,734,809 SEQ ID NO: 71 |
| AAV CLv-8 | 675 | U.S. Pat. No. 8,734,809 SEQ ID NO: 72 |
| AAV CKd-B1 | 676 | U.S. Pat. No. 8,734,809 SEQ ID NO: 73 |
| AAV CKd-B2 | 677 | U.S. Pat. No. 8,734,809 SEQ ID NO: 74 |
| AAV CKd-B3 | 678 | U.S. Pat. No. 8,734,809 SEQ ID NO: 75 |
| AAV CKd-B4 | 679 | U.S. Pat. No. 8,734,809 SEQ ID NO: 76 |
| AAV CKd-B5 | 680 | U.S. Pat. No. 8,734,809 SEQ ID NO: 77 |
| AAV CKd-B6 | 681 | U.S. Pat. No. 8,734,809 SEQ ID NO: 78 |
| AAV CKd-B7 | 682 | U.S. Pat. No. 8,734,809 SEQ ID NO: 79 |
| AAV CKd-B8 | 683 | U.S. Pat. No. 8,734,809 SEQ ID NO: 80 |
| AAV CKd-H1 | 684 | U.S. Pat. No. 8,734,809 SEQ ID NO: 81 |
| AAV CKd-H2 | 685 | U.S. Pat. No. 8,734,809 SEQ ID NO: 82 |
| AAV CKd-H3 | 686 | U.S. Pat. No. 8,734,809 SEQ ID NO: 83 |
| AAV CKd-H4 | 687 | U.S. Pat. No. 8,734,809 SEQ ID NO: 84 |
| AAV CKd-H5 | 688 | U.S. Pat. No. 8,734,809 SEQ ID NO: 85 |
| AAV CKd-H6 | 689 | U.S. Pat. No. 8,734,809 SEQ ID NO: 77 |
| AAV CHt-1 | 690 | U.S. Pat. No. 8,734,809 SEQ ID NO: 86 |
| AAV CLv1-1 | 691 | U.S. Pat. No. 8,734,809 SEQ ID NO: 171 |
| AAV CLv1-2 | 692 | U.S. Pat. No. 8,734,809 SEQ ID NO: 172 |
| AAV CLv1-3 | 693 | U.S. Pat. No. 8,734,809 SEQ ID NO: 173 |
| AAV CLv1-4 | 694 | U.S. Pat. No. 8,734,809 SEQ ID NO: 174 |
| AAV Clv1-7 | 695 | U.S. Pat. No. 8,734,809 SEQ ID NO: 175 |
| AAV Clv1-8 | 696 | U.S. Pat. No. 8,734,809 SEQ ID NO: 176 |
| AAV Clv 1-9 | 697 | U.S. Pat. No. 8,734,809 SEQ ID NO: 177 |
| AAV Clv1-10 | 698 | U.S. Pat. No. 8,734,809 SEQ ID NO: 178 |
| AAV.VR-355 | 699 | U.S. Pat. No. 8,734,809 SEQ ID NO: 181 |
| AAV.hu.48R3 | 700 | U.S. Pat. No. 8,734,809 SEQ ID NO: 183 |
| AAV CBr-E1 | 701 | U.S. Pat. No. 8,734,809 SEQ ID NO: 87 |
| AAV CBr-E2 | 702 | U.S. Pat. No. 8,734,809 SEQ ID NO: 88 |
| AAV CBr-E3 | 703 | U.S. Pat. No. 8,734,809 SEQ ID NO: 89 |
| AAV CBr-E4 | 704 | U.S. Pat. No. 8,734,809 SEQ ID NO: 90 |
| AAV CBr-E5 | 705 | U.S. Pat. No. 8,734,809 SEQ ID NO: 91 |
| AAV CBr-e5 | 706 | U.S. Pat. No. 8,734,809 SEQ ID NO: 92 |
| AAV CBr-E6 | 707 | U.S. Pat. No. 8,734,809 SEQ ID NO: 93 |
| AAV CBr-E7 | 708 | U.S. Pat. No. 8,734,809 SEQ ID NO: 94 |
| AAV CBr-E8 | 709 | U.S. Pat. No. 8,734,809 SEQ ID NO: 95 |
| AAV CLv-D1 | 710 | U.S. Pat. No. 8,734,809 SEQ ID NO: 96 |
| AAV CLv-D2 | 711 | U.S. Pat. No. 8,734,809 SEQ ID NO: 97 |
| AAV CLv-D3 | 712 | U.S. Pat. No. 8,734,809 SEQ ID NO: 98 |
| AAV CLv-D4 | 713 | U.S. Pat. No. 8,734,809 SEQ ID NO: 99 |
| AAV CLv-D5 | 714 | U.S. Pat. No. 8,734,809 SEQ ID NO: 100 |
| AAV CLv-D6 | 715 | U.S. Pat. No. 8,734,809 SEQ ID NO: 101 |
| AAV CLv-D7 | 716 | U.S. Pat. No. 8,734,809 SEQ ID NO: 102 |
| AAV CLv-D8 | 717 | U.S. Pat. No. 8,734,809 SEQ ID NO: 103 |
| AAV CLv-E1 | 718 | U.S. Pat. No. 8,734,809 SEQ ID NO: 87 |
| AAV CLv-R1 | 719 | U.S. Pat. No. 8,734,809 SEQ ID NO: 104 |
| AAV CLv-R2 | 720 | U.S. Pat. No. 8,734,809 SEQ ID NO: 105 |
| AAV CLv-R3 | 721 | U.S. Pat. No. 8,734,809 SEQ ID NO: 106 |
| AAV CLv-R4 | 722 | U.S. Pat. No. 8,734,809 SEQ ID NO: 107 |
| AAV CLv-R5 | 723 | U.S. Pat. No. 8,734,809 SEQ ID NO: 108 |
| AAV CLv-R6 | 724 | U.S. Pat. No. 8,734,809 SEQ ID NO: 109 |
| AAV CLv-R7 | 725 | U.S. Pat. No. 8,734,809 SEQ ID NO: 110 |
| AAV CLv-R8 | 726 | U.S. Pat. No. 8,734,809 SEQ ID NO: 111 |
| AAV CLv-R9 | 727 | U.S. Pat. No. 8,734,809 SEQ ID NO: 112 |
| AAV CLg-F1 | 728 | U.S. Pat. No. 8,734,809 SEQ ID NO: 113 |
| AAV CLg-F2 | 729 | U.S. Pat. No. 8,734,809 SEQ ID NO: 114 |
| AAV CLg-F3 | 730 | U.S. Pat. No. 8,734,809 SEQ ID NO: 115 |
| AAV CLg-F4 | 731 | U.S. Pat. No. 8,734,809 SEQ ID NO: 116 |
| AAV CLg-F5 | 732 | U.S. Pat. No. 8,734,809 SEQ ID NO: 117 |
| AAV CLg-F6 | 733 | U.S. Pat. No. 8,734,809 SEQ ID NO: 117 |
| AAV CLg-F7 | 734 | U.S. Pat. No. 8,734,809 SEQ ID NO: 118 |
| AAV CLg-F8 | 735 | U.S. Pat. No. 8,734,809 SEQ ID NO: 117 |
| AAV CSp-1 | 736 | U.S. Pat. No. 8,734,809 SEQ ID NO: 119 |
| AAV CSp-10 | 737 | U.S. Pat. No. 8,734,809 SEQ ID NO: 120 |
| AAV CSp-11 | 738 | U.S. Pat. No. 8,734,809 SEQ ID NO: 121 |
| AAV CSp-2 | 739 | U.S. Pat. No. 8,734,809 SEQ ID NO: 122 |
| AAV CSp-3 | 740 | U.S. Pat. No. 8,734,809 SEQ ID NO: 123 |
| AAV CSp-4 | 741 | U.S. Pat. No. 8,734,809 SEQ ID NO: 124 |
| AAV CSp-6 | 742 | U.S. Pat. No. 8,734,809 SEQ ID NO: 125 |
| AAV CSp-7 | 743 | U.S. Pat. No. 8,734,809 SEQ ID NO: 126 |
| AAV CSp-8 | 744 | U.S. Pat. No. 8,734,809 SEQ ID NO: 127 |
| AAV CSp-9 | 745 | U.S. Pat. No. 8,734,809 SEQ ID NO: 128 |
| AAV CHt-2 | 746 | U.S. Pat. No. 8,734,809 SEQ ID NO: 129 |
| AAV CHt-3 | 747 | U.S. Pat. No. 8,734,809 SEQ ID NO: 130 |
| AAV CKd-1 | 748 | U.S. Pat. No. 8,734,809 SEQ ID NO: 131 |
| AAV CKd-10 | 749 | U.S. Pat. No. 8,734,809 SEQ ID NO: 132 |
| AAV CKd-2 | 750 | U.S. Pat. No. 8,734,809 SEQ ID NO: 133 |
| AAV CKd-3 | 751 | U.S. Pat. No. 8,734,809 SEQ ID NO: 134 |
| AAV CKd-4 | 752 | U.S. Pat. No. 8,734,809 SEQ ID NO: 135 |
| AAV CKd-6 | 753 | U.S. Pat. No. 8,734,809 SEQ ID NO: 136 |
| AAV CKd-7 | 754 | U.S. Pat. No. 8,734,809 SEQ ID NO: 137 |
| AAV CKd-8 | 755 | U.S. Pat. No. 8,734,809 SEQ ID NO: 138 |
| AAV CLv-1 | 756 | U.S. Pat. No. 8,734,809 SEQ ID NO: 139 |
| AAV CLv-12 | 757 | U.S. Pat. No. 8,734,809 SEQ ID NO: 140 |
| AAV CLv-13 | 758 | U.S. Pat. No. 8,734,809 SEQ ID NO: 141 |
| AAV CLv-2 | 759 | U.S. Pat. No. 8,734,809 SEQ ID NO: 142 |
| AAV CLv-3 | 760 | U.S. Pat. No. 8,734,809 SEQ ID NO: 143 |
| AAV CLv-4 | 761 | U.S. Pat. No. 8,734,809 SEQ ID NO: 144 |
| AAV CLv-6 | 762 | U.S. Pat. No. 8,734,809 SEQ ID NO: 145 |
| AAV CLv-8 | 763 | U.S. Pat. No. 8,734,809 SEQ ID NO: 146 |
| AAV CKd-B1 | 764 | U.S. Pat. No. 8,734,809 SEQ ID NO: 147 |
| AAV CKd-B2 | 765 | U.S. Pat. No. 8,734,809 SEQ ID NO: 148 |
| AAV CKd-B3 | 766 | U.S. Pat. No. 8,734,809 SEQ ID NO: 149 |
| AAV CKd-B4 | 767 | U.S. Pat. No. 8,734,809 SEQ ID NO: 150 |
| AAV CKd-B5 | 768 | U.S. Pat. No. 8,734,809 SEQ ID NO: 151 |
| AAV CKd-B6 | 769 | U.S. Pat. No. 8,734,809 SEQ ID NO: 152 |
| AAV CKd-B7 | 770 | U.S. Pat. No. 8,734,809 SEQ ID NO: 153 |
| AAV CKd-B8 | 771 | U.S. Pat. No. 8,734,809 SEQ ID NO: 154 |
| AAV CKd-H1 | 772 | U.S. Pat. No. 8,734,809 SEQ ID NO: 155 |
| AAV CKd-H2 | 773 | U.S. Pat. No. 8,734,809 SEQ ID NO: 156 |
| AAV CKd-H3 | 774 | U.S. Pat. No. 8,734,809 SEQ ID NO: 157 |
| AAV CKd-H4 | 775 | U.S. Pat. No. 8,734,809 SEQ ID NO: 158 |
| AAV CKd-H5 | 776 | U.S. Pat. No. 8,734,809 SEQ ID NO: 159 |
| AAV CKd-H6 | 777 | U.S. Pat. No. 8,734,809 SEQ ID NO: 151 |
| AAV CHt-1 | 778 | U.S. Pat. No. 8,734,809 SEQ ID NO: 160 |
| AAV CHt-P2 | 779 | WO2016065001 SEQ ID NO: 1 |
| AAV CHt-P5 | 780 | WO2016065001 SEQ ID NO: 2 |
| AAV CHt-P9 | 781 | WO2016065001 SEQ ID NO: 3 |
| AAV CBr-7.1 | 782 | WO2016065001 SEQ ID NO: 4 |
| AAV CBr-7.2 | 783 | WO2016065001 SEQ ID NO: 5 |
| AAV CBr-7.3 | 784 | WO2016065001 SEQ ID NO: 6 |
| AAV CBr-7.4 | 785 | WO2016065001 SEQ ID NO: 7 |
| AAV CBr-7.5 | 786 | WO2016065001 SEQ ID NO: 8 |
| AAV CBr-7.7 | 787 | WO2016065001 SEQ ID NO: 9 |
| AAV CBr-7.8 | 788 | WO2016065001 SEQ ID NO: 10 |
| AAV CBr-7.10 | 789 | WO2016065001 SEQ ID NO: 11 |
| AAV CKd-N3 | 790 | WO2016065001 SEQ ID NO: 12 |
| AAV CKd-N4 | 791 | WO2016065001 SEQ ID NO: 13 |
| AAV CKd-N9 | 792 | WO2016065001 SEQ ID NO: 14 |
| AAV CLv-L4 | 793 | WO2016065001 SEQ ID NO: 15 |
| AAV CLv-L5 | 794 | WO2016065001 SEQ ID NO: 16 |
| AAV CLv-L6 | 795 | WO2016065001 SEQ ID NO: 17 |
| AAV CLv-K1 | 796 | WO2016065001 SEQ ID NO: 18 |
| AAV CLv-K3 | 797 | WO2016065001 SEQ ID NO: 19 |
| AAV CLv-K6 | 798 | WO2016065001 SEQ ID NO: 20 |
| AAV CLv-M1 | 799 | WO2016065001 SEQ ID NO: 21 |
| AAV CLv-M11 | 800 | WO2016065001 SEQ ID NO: 22 |
| AAV CLv-M2 | 801 | WO2016065001 SEQ ID NO: 23 |
| AAV CLv-M5 | 802 | WO2016065001 SEQ ID NO: 24 |
| AAV CLv-M6 | 803 | WO2016065001 SEQ ID NO: 25 |
| AAV CLv-M7 | 804 | WO2016065001 SEQ ID NO: 26 |
| AAV CLv-M8 | 805 | WO2016065001 SEQ ID NO: 27 |
| AAV CLv-M9 | 806 | WO2016065001 SEQ ID NO: 28 |
| AAV CHt-P1 | 807 | WO2016065001 SEQ ID NO: 29 |
| AAV CHt-P6 | 808 | WO2016065001 SEQ ID NO: 30 |
| AAV CHt-P8 | 809 | WO2016065001 SEQ ID NO: 31 |
| AAV CHt-6.1 | 810 | WO2016065001 SEQ ID NO: 32 |
| AAV CHt-6.10 | 811 | WO2016065001 SEQ ID NO: 33 |
| AAV CHt-6.5 | 812 | WO2016065001 SEQ ID NO: 34 |
| AAV CHt-6.6 | 813 | WO2016065001 SEQ ID NO: 35 |
| AAV CHt-6.7 | 814 | WO2016065001 SEQ ID NO: 36 |
| AAV CHt-6.8 | 815 | WO2016065001 SEQ ID NO: 37 |
| AAV CSp-8.10 | 816 | WO2016065001 SEQ ID NO: 38 |
| AAV CSp-8.2 | 817 | WO2016065001 SEQ ID NO: 39 |
| AAV CSp-8.4 | 818 | WO2016065001 SEQ ID NO: 40 |
| AAV CSp-8.5 | 819 | WO2016065001 SEQ ID NO: 41 |
| AAV CSp-8.6 | 820 | WO2016065001 SEQ ID NO: 42 |
| AAV CSp-8.7 | 821 | WO2016065001 SEQ ID NO: 43 |
| AAV CSp-8.8 | 822 | WO2016065001 SEQ ID NO: 44 |
| AAV CSp-8.9 | 823 | WO2016065001 SEQ ID NO: 45 |
| AAV CBr-B7.3 | 824 | WO2016065001 SEQ ID NO: 46 |
| AAV CBr-B7.4 | 825 | WO2016065001 SEQ ID NO: 47 |
| AAV3B | 826 | WO2016065001 SEQ ID NO: 48 |
| AAV4 | 827 | WO2016065001 SEQ ID NO: 49 |
| AAV5 | 828 | WO2016065001 SEQ ID NO: 50 |
| AAV CHt-P2 | 829 | WO2016065001 SEQ ID NO: 51 |
| AAV CHt-P5 | 830 | WO2016065001 SEQ ID NO: 52 |
| AAV CHt-P9 | 831 | WO2016065001 SEQ ID NO: 53 |
| AAV CBr-7.1 | 832 | WO2016065001 SEQ ID NO: 54 |
| AAV CBr-7.2 | 833 | WO2016065001 SEQ ID NO: 55 |
| AAV CBr-7.3 | 834 | WO2016065001 SEQ ID NO: 56 |
| AAV CBr-7.4 | 835 | WO2016065001 SEQ ID NO: 57 |
| AAV CBr-7.5 | 836 | WO2016065001 SEQ ID NO: 58 |
| AAV CBr-7.7 | 837 | WO2016065001 SEQ ID NO: 59 |
| AAV CBr-7.8 | 838 | WO2016065001 SEQ ID NO: 60 |
| AAV CBr-7.10 | 839 | WO2016065001 SEQ ID NO: 61 |
| AAV CKd-N3 | 840 | WO2016065001 SEQ ID NO: 62 |
| AAV CKd-N4 | 841 | WO2016065001 SEQ ID NO: 63 |
| AAV CKd-N9 | 842 | WO2016065001 SEQ ID NO: 64 |
| AAV CLv-L4 | 843 | WO2016065001 SEQ ID NO: 65 |
| AAV CLv-L5 | 844 | WO2016065001 SEQ ID NO: 66 |
| AAV CLv-L6 | 845 | WO2016065001 SEQ ID NO: 67 |
| AAV CLv-K1 | 846 | WO2016065001 SEQ ID NO: 68 |
| AAV CLv-K3 | 847 | WO2016065001 SEQ ID NO: 69 |
| AAV CLv-K6 | 848 | WO2016065001 SEQ ID NO: 70 |
| AAV CLv-M1 | 849 | WO2016065001 SEQ ID NO: 71 |
| AAV CLv-M11 | 850 | WO2016065001 SEQ ID NO: 72 |
| AAV CLv-M2 | 851 | WO2016065001 SEQ ID NO: 73 |
| AAV CLv-M5 | 852 | WO2016065001 SEQ ID NO: 74 |
| AAV CLv-M6 | 853 | WO2016065001 SEQ ID NO: 75 |
| AAV CLv-M7 | 854 | WO2016065001 SEQ ID NO: 76 |
| AAV CLv-M8 | 855 | WO2016065001 SEQ ID NO: 77 |
| AAV CLv-M9 | 856 | WO2016065001 SEQ ID NO: 78 |
| AAV CHt-P1 | 857 | WO2016065001 SEQ ID NO: 79 |
| AAV CHt-P6 | 858 | WO2016065001 SEQ ID NO: 80 |
| AAV CHt-P8 | 859 | WO2016065001 SEQ ID NO: 81 |
| AAV CHt-6.1 | 860 | WO2016065001 SEQ ID NO: 82 |
| AAV CHt-6.10 | 861 | WO2016065001 SEQ ID NO: 83 |
| AAV CHt-6.5 | 862 | WO2016065001 SEQ ID NO: 84 |
| AAV CHt-6.6 | 863 | WO2016065001 SEQ ID NO: 85 |
| AAV CHt-6.7 | 864 | WO2016065001 SEQ ID NO: 86 |
| AAV CHt-6.8 | 865 | WO2016065001 SEQ ID NO: 87 |
| AAV CSp-8.10 | 866 | WO2016065001 SEQ ID NO: 88 |
| AAV CSp-8.2 | 867 | WO2016065001 SEQ ID NO: 89 |
| AAV CSp-8.4 | 868 | WO2016065001 SEQ ID NO: 90 |
| AAV CSp-8.5 | 869 | WO2016065001 SEQ ID NO: 91 |
| AAV CSp-8.6 | 870 | WO2016065001 SEQ ID NO: 92 |
| AAV CSp-8.7 | 871 | WO2016065001 SEQ ID NO: 93 |
| AAV CSp-8.8 | 872 | WO2016065001 SEQ ID NO: 94 |
| AAV CSp-8.9 | 873 | WO2016065001 SEQ ID NO: 95 |
| AAV CBr-B7.3 | 874 | WO2016065001 SEQ ID NO: 96 |
| AAV CBr-B7.4 | 875 | WO2016065001 SEQ ID NO: 97 |
| AAV3B | 876 | WO2016065001 SEQ ID NO: 98 |
| AAV4 | 877 | WO2016065001 SEQ ID NO: 99 |
| AAV5 | 878 | WO2016065001 SEQ ID NO: 100 |
| GPV | 879 | U.S. Pat. No. 9,624,274B2 SEQ ID NO: 192 |
| B19 | 880 | U.S. Pat. No. 9,624,274B2 SEQ ID NO: 193 |
| MVM | 881 | U.S. Pat. No. 9,624,274B2 SEQ ID NO: 194 |
| FPV | 882 | U.S. Pat. No. 9,624,274B2 SEQ ID NO: 195 |
| CPV | 883 | U.S. Pat. No. 9,624,274B2 SEQ ID NO: 196 |
| AAV6 | 884 | U.S. Pat. No. 9,546,112B2 SEQ ID NO: 5 |
| AAV6 | 885 | U.S. Pat. No. 9,457,103B2 SEQ ID NO: 1 |
| AAV2 | 886 | U.S. Pat. No. 9,457,103B2 SEQ ID NO: 2 |
| ShH10 | 887 | U.S. Pat. No. 9,457,103B2 SEQ ID NO: 3 |
| ShH13 | 888 | U.S. Pat. No. 9,457,103B2 SEQ ID NO: 4 |
| ShH10 | 889 | U.S. Pat. No. 9,457,103B2 SEQ ID NO: 5 |
| ShH10 | 890 | U.S. Pat. No. 9,457,103B2 SEQ ID NO: 6 |
| ShH10 | 891 | U.S. Pat. No. 9,457,103B2 SEQ ID NO: 7 |
| ShH10 | 892 | U.S. Pat. No. 9,457,103B2 SEQ ID NO: 8 |
| ShH10 | 893 | U.S. Pat. No. 9,457,103B2 SEQ ID NO: 9 |
| rh74 | 894 | U.S. Pat. No. 9,434,928B2 SEQ ID NO: 1, US2015023924A1 SEQ ID NO: 2 |
| rh74 | 895 | U.S. Pat. No. 9,434,928B2 SEQ ID NO: 2, US2015023924A1 SEQ ID NO: 1 |
| AAV8 | 896 | U.S. Pat. No. 9,434,928B2 SEQ ID NO: 4 |
| rh74 | 897 | U.S. Pat. No. 9,434,928B2 SEQ ID NO: 5 |
| rh74 (RHM4-1) | 898 | US2015023924A1 SEQ ID NO: 5, US20160375110A1 SEQ ID NO: 4 |
| rh74 (RHM15-1) | 899 | US2015023924A1 SEQ ID NO: 6, US20160375110A1 SEQ ID NO: 5 |
| rh74 (RHM15-2) | 900 | US2015023924A1 SEQ ID NO: 7, US20160375110A1 SEQ ID NO: 6 |
| rh74 (RHM15-3/RHM15-5) | 901 | US2015023924A1 SEQ ID NO: 8, US20160375110A1 SEQ ID NO: 7 |
| rh74 (RHM15-4) | 902 | US2015023924A1 SEQ ID NO: 9, US20160375110A1 SEQ ID NO: 8 |
| rh74 (RHM15-6) | 903 | US2015023924A1 SEQ ID NO: 10, US20160375110A1 SEQ ID NO: 9 |
| rh74 (RHM4-1) | 904 | US2015023924A1 SEQ ID NO: 11 |
| rh74 (RHM15-1) | 905 | US2015023924A1 SEQ ID NO: 12 |
| rh74 (RHM15-2) | 906 | US2015023924A1 SEQ ID NO: 13 |
| rh74 (RHM15-3/RHM15-5) | 907 | US2015023924A1 SEQ ID NO: 14 |
| rh74 (RHM15-4) | 908 | US2015023924A1 SEQ ID NO: 15 |
| rh74 (RHM15-6) | 909 | US2015023924A1 SEQ ID NO: 16 |
| AAV2 (comprising lung | 910 | US20160175389A1 SEQ ID NO: 9 |
| specific polypeptide) | | |
| AAV2 (comprising lung | 911 | US20160175389A1 SEQ ID NO: 10 |
| specific polypeptide) | | |
| Anc80 | 912 | US20170051257A1 SEQ ID NO: 1 |
| Anc80 | 913 | US20170051257A1 SEQ ID NO: 2 |
| Anc81 | 914 | US20170051257A1 SEQ ID NO: 3 |
| Anc80 | 915 | US20170051257A1 SEQ ID NO: 4 |
| Anc82 | 916 | US20170051257A1 SEQ ID NO: 5 |
| Anc82 | 917 | US20170051257A1 SEQ ID NO: 6 |
| Anc83 | 918 | US20170051257A1 SEQ ID NO: 7 |
| Anc83 | 919 | US20170051257A1 SEQ ID NO: 8 |
| Anc84 | 920 | US20170051257A1 SEQ ID NO: 9 |
| Anc84 | 921 | US20170051257A1 SEQ ID NO: 10 |
| Anc94 | 922 | US20170051257A1 SEQ ID NO: 11 |
| Anc94 | 923 | US20170051257A1 SEQ ID NO: 12 |
| Anc113 | 924 | US20170051257A1 SEQ ID NO: 13 |
| Anc113 | 925 | US20170051257A1 SEQ ID NO: 14 |
| Anc126 | 926 | US20170051257A1 SEQ ID NO: 15 |
| Anc126 | 927 | US20170051257A1 SEQ ID NO: 16 |
| Anc127 | 928 | US20170051257A1 SEQ ID NO: 17 |
| Anc127 | 929 | US20170051257A1 SEQ ID NO: 18 |
| Anc80L27 | 930 | US20170051257A1 SEQ ID NO: 19 |
| Anc80L59 | 931 | US20170051257A1 SEQ ID NO: 20 |
| Anc80L60 | 932 | US20170051257A1 SEQ ID NO: 21 |
| Anc80L62 | 933 | US20170051257A1 SEQ ID NO: 22 |
| Anc80L65 | 934 | US20170051257A1 SEQ ID NO: 23 |
| Anc80L33 | 935 | US20170051257A1 SEQ ID NO: 24 |
| Anc80L36 | 936 | US20170051257A1 SEQ ID NO: 25 |
| Anc80L44 | 937 | US20170051257A1 SEQ ID NO: 26 |
| Anc80L1 | 938 | US20170051257A1 SEQ ID NO: 35 |
| Anc80L1 | 939 | US20170051257A1 SEQ ID NO: 36 |
| AAV-X1 | 940 | U.S. Pat. No. 8,283,151B2 SEQ ID NO: 11 |
| AAV-X1b | 941 | U.S. Pat. No. 8,283,151B2 SEQ ID NO: 12 |
| AAV-X5 | 942 | U.S. Pat. No. 8,283,151B2 SEQ ID NO: 13 |
| AAV-X19 | 943 | U.S. Pat. No. 8,283,151B2 SEQ ID NO: 14 |
| AAV-X21 | 944 | U.S. Pat. No. 8,283,151B2 SEQ ID NO: 15 |
| AAV-X22 | 945 | U.S. Pat. No. 8,283,151B2 SEQ ID NO: 16 |
| AAV-X23 | 946 | U.S. Pat. No. 8,283,151B2 SEQ ID NO: 17 |
| AAV-X24 | 947 | U.S. Pat. No. 8,283,151B2 SEQ ID NO: 18 |
| AAV-X25 | 948 | U.S. Pat. No. 8,283,151B2 SEQ ID NO: 19 |
| AAV-X26 | 949 | U.S. Pat. No. 8,283,151B2 SEQ ID NO: 20 |
| AAV-X1 | 950 | U.S. Pat. No. 8,283,151B2 SEQ ID NO: 21 |
| AAV-X1b | 951 | U.S. Pat. No. 8,283,151B2 SEQ ID NO: 22 |
| AAV-X5 | 952 | U.S. Pat. No. 8,283,151B2 SEQ ID NO: 23 |
| AAV-X19 | 953 | U.S. Pat. No. 8,283,151B2 SEQ ID NO: 24 |
| AAV-X21 | 954 | U.S. Pat. No. 8,283,151B2 SEQ ID NO: 25 |
| AAV-X22 | 955 | U.S. Pat. No. 8,283,151B2 SEQ ID NO: 26 |
| AAV-X23 | 956 | U.S. Pat. No. 8,283,151B2 SEQ ID NO: 27 |
| AAV-X24 | 957 | U.S. Pat. No. 8,283,151B2 SEQ ID NO: 28 |
| AAV-X25 | 958 | U.S. Pat. No. 8,283,151B2 SEQ ID NO: 29 |
| AAV-X26 | 959 | U.S. Pat. No. 8,283,151B2 SEQ ID NO: 30 |
| AAVrh8 | 960 | WO2016054554A1 SEQ ID NO: 8 |
| AAVrh8VP2FC5 | 961 | WO2016054554A1 SEQ ID NO: 9 |
| AAVrh8VP2FC44 | 962 | WO2016054554A1 SEQ ID NO: 10 |
| AAVrh8VP2ApoB100 | 963 | WO2016054554A1 SEQ ID NO: 11 |
| AAVrh8VP2RVG | 964 | WO2016054554A1 SEQ ID NO: 12 |
| AAVrh8VP2Angiopep-2 VP2 | 965 | WO2016054554A1 SEQ ID NO: 13 |
| AAV9.47VP1.3 | 966 | WO2016054554A1 SEQ ID NO: 14 |
| AAV9.47VP2ICAMg3 | 967 | WO2016054554A1 SEQ ID NO: 15 |
| AAV9.47VP2RVG | 968 | WO2016054554A1 SEQ ID NO: 16 |
| AAV9.47VP2Angiopep-2 | 969 | WO2016054554A1 SEQ ID NO: 17 |
| AAV9.47VP2A-string | 970 | WO2016054554A1 SEQ ID NO: 18 |
| AAVrh8VP2FC5 VP2 | 971 | WO2016054554A1 SEQ ID NO: 19 |
| AAVrh8VP2FC44 VP2 | 972 | WO2016054554A1 SEQ ID NO: 20 |
| AAVrh8VP2ApoB100 VP2 | 973 | WO2016054554A1 SEQ ID NO: 21 |
| AAVrh8VP2RVG VP2 | 974 | WO2016054554A1 SEQ ID NO: 22 |
| AAVrh8VP2Angiopep-2 VP2 | 975 | WO2016054554A1 SEQ ID NO: 23 |
| AAV9.47VP2ICAMg3 VP2 | 976 | WO2016054554A1 SEQ ID NO: 24 |
| AAV9.47VP2RVG VP2 | 977 | WO2016054554A1 SEQ ID NO: 25 |
| AAV9.47VP2Angiopep-2 VP2 | 978 | WO2016054554A1 SEQ ID NO: 26 |
| AAV9.47VP2A-string VP2 | 979 | WO2016054554A1 SEQ ID NO: 27 |
| rAAV-B1 | 980 | WO2016054557A1 SEQ ID NO: 1 |
| rAAV-B2 | 981 | WO2016054557A1 SEQ ID NO: 2 |
| rAAV-B3 | 982 | WO2016054557A1 SEQ ID NO: 3 |
| rAAV-B4 | 983 | WO2016054557A1 SEQ ID NO: 4 |
| rAAV-B1 | 984 | WO2016054557A1 SEQ ID NO: 5 |
| rAAV-B2 | 985 | WO2016054557A1 SEQ ID NO: 6 |
| rAAV-B3 | 986 | WO2016054557A1 SEQ ID NO: 7 |
| rAAV-B4 | 987 | WO2016054557A1 SEQ ID NO: 8 |
| rAAV-L1 | 988 | WO2016054557A1 SEQ ID NO: 9 |
| rAAV-L2 | 989 | WO2016054557A1 SEQ ID NO: 10 |
| rAAV-L3 | 990 | WO2016054557A1 SEQ ID NO: 11 |
| rAAV-L4 | 991 | WO2016054557A1 SEQ ID NO: 12 |
| rAAV-L1 | 992 | WO2016054557A1 SEQ ID NO: 13 |
| rAAV-L2 | 993 | WO2016054557A1 SEQ ID NO: 14 |
| rAAV-L3 | 994 | WO2016054557A1 SEQ ID NO: 15 |
| rAAV-L4 | 995 | WO2016054557A1 SEQ ID NO: 16 |
| AAV9 | 996 | WO2016073739A1 SEQ ID NO: 3 |
| rAAV | 997 | WO2016081811A1 SEQ ID NO: 1 |
| rAAV | 998 | WO2016081811A1 SEQ ID NO: 2 |
| rAAV | 999 | WO2016081811A1 SEQ ID NO: 3 |
| rAAV | 1000 | WO2016081811A1 SEQ ID NO: 4 |
| rAAV | 1001 | WO2016081811A1 SEQ ID NO: 5 |
| rAAV | 1002 | WO2016081811A1 SEQ ID NO: 6 |
| rAAV | 1003 | WO2016081811A1 SEQ ID NO: 7 |
| rAAV | 1004 | WO2016081811A1 SEQ ID NO: 8 |
| rAAV | 1005 | WO2016081811A1 SEQ ID NO: 9 |
| rAAV | 1006 | WO2016081811A1 SEQ ID NO: 10 |
| rAAV | 1007 | WO2016081811A1 SEQ ID NO: 11 |
| rAAV | 1008 | WO2016081811A1 SEQ ID NO: 12 |
| rAAV | 1009 | WO2016081811A1 SEQ ID NO: 13 |
| rAAV | 1010 | WO2016081811A1 SEQ ID NO: 14 |
| rAAV | 1011 | WO2016081811A1 SEQ ID NO: 15 |
| rAAV | 1012 | WO2016081811A1 SEQ ID NO: 16 |
| rAAV | 1013 | WO2016081811A1 SEQ ID NO: 17 |
| rAAV | 1014 | WO2016081811A1 SEQ ID NO: 18 |
| rAAV | 1015 | WO2016081811A1 SEQ ID NO: 19 |
| rAAV | 1016 | WO2016081811A1 SEQ ID NO: 20 |
| rAAV | 1017 | WO2016081811A1 SEQ ID NO: 21 |
| rAAV | 1018 | WO2016081811A1 SEQ ID NO: 22 |
| rAAV | 1019 | WO2016081811A1 SEQ ID NO: 23 |
| rAAV | 1020 | WO2016081811A1 SEQ ID NO: 24 |
| rAAV | 1021 | WO2016081811A1 SEQ ID NO: 25 |
| rAAV | 1022 | WO2016081811A1 SEQ ID NO: 26 |
| rAAV | 1023 | WO2016081811A1 SEQ ID NO: 27 |
| rAAV | 1024 | WO2016081811A1 SEQ ID NO: 28 |
| rAAV | 1025 | WO2016081811A1 SEQ ID NO: 29 |
| rAAV | 1026 | WO2016081811A1 SEQ ID NO: 30 |
| rAAV | 1027 | WO2016081811A1 SEQ ID NO: 31 |
| rAAV | 1028 | WO2016081811A1 SEQ ID NO: 32 |
| rAAV | 1029 | WO2016081811A1 SEQ ID NO: 33 |
| rAAV | 1030 | WO2016081811A1 SEQ ID NO: 34 |
| rAAV | 1031 | WO2016081811A1 SEQ ID NO: 35 |
| rAAV | 1032 | WO2016081811A1 SEQ ID NO: 36 |
| rAAV | 1033 | WO2016081811A1 SEQ ID NO: 37 |
| rAAV | 1034 | WO2016081811A1 SEQ ID NO: 38 |
| rAAV | 1035 | WO2016081811A1 SEQ ID NO: 39 |
| rAAV | 1036 | WO2016081811A1 SEQ ID NO: 40 |
| rAAV | 1037 | WO2016081811A1 SEQ ID NO: 41 |
| rAAV | 1038 | WO2016081811A1 SEQ ID NO: 42 |
| rAAV | 1039 | WO2016081811A1 SEQ ID NO: 43 |
| rAAV | 1040 | WO2016081811A1 SEQ ID NO: 44 |
| rAAV | 1041 | WO2016081811A1 SEQ ID NO: 45 |
| rAAV | 1042 | WO2016081811A1 SEQ ID NO: 46 |
| rAAV | 1043 | WO2016081811A1 SEQ ID NO: 47 |
| rAAV | 1044 | WO2016081811A1 SEQ ID NO: 48 |
| rAAV | 1045 | WO2016081811A1 SEQ ID NO: 49 |
| rAAV | 1046 | WO2016081811A1 SEQ ID NO: 50 |
| rAAV | 1047 | WO2016081811A1 SEQ ID NO: 51 |
| rAAV | 1048 | WO2016081811A1 SEQ ID NO: 52 |
| rAAV | 1049 | WO2016081811A1 SEQ ID NO: 53 |
| rAAV | 1050 | WO2016081811A1 SEQ ID NO: 54 |
| rAAV | 1051 | WO2016081811A1 SEQ ID NO: 55 |
| rAAV | 1052 | WO2016081811A1 SEQ ID NO: 56 |
| rAAV | 1053 | WO2016081811A1 SEQ ID NO: 57 |
| rAAV | 1054 | WO2016081811A1 SEQ ID NO: 58 |
| rAAV | 1055 | WO2016081811A1 SEQ ID NO: 59 |
| rAAV | 1056 | WO2016081811A1 SEQ ID NO: 60 |
| rAAV | 1057 | WO2016081811A1 SEQ ID NO: 61 |
| rAAV | 1058 | WO2016081811A1 SEQ ID NO: 62 |
| rAAV | 1059 | WO2016081811A1 SEQ ID NO: 63 |
| rAAV | 1060 | WO2016081811A1 SEQ ID NO: 64 |
| rAAV | 1061 | WO2016081811A1 SEQ ID NO: 65 |
| rAAV | 1062 | WO2016081811A1 SEQ ID NO: 66 |
| rAAV | 1063 | WO2016081811A1 SEQ ID NO: 67 |
| rAAV | 1064 | WO2016081811A1 SEQ ID NO: 68 |
| rAAV | 1065 | WO2016081811A1 SEQ ID NO: 69 |
| rAAV | 1066 | WO2016081811A1 SEQ ID NO: 70 |
| rAAV | 1067 | WO2016081811A1 SEQ ID NO: 71 |
| rAAV | 1068 | WO2016081811A1 SEQ ID NO: 72 |
| rAAV | 1069 | WO2016081811A1 SEQ ID NO: 73 |
| rAAV | 1070 | WO2016081811A1 SEQ ID NO: 74 |
| rAAV | 1071 | WO2016081811A1 SEQ ID NO: 75 |
| rAAV | 1072 | WO2016081811A1 SEQ ID NO: 76 |
| rAAV | 1073 | WO2016081811A1 SEQ ID NO: 77 |
| rAAV | 1074 | WO2016081811A1 SEQ ID NO: 78 |
| rAAV | 1075 | WO2016081811A1 SEQ ID NO: 79 |
| rAAV | 1076 | WO2016081811A1 SEQ ID NO: 80 |
| rAAV | 1077 | WO2016081811A1 SEQ ID NO: 81 |
| rAAV | 1078 | WO2016081811A1 SEQ ID NO: 82 |
| rAAV | 1079 | WO2016081811A1 SEQ ID NO: 83 |
| rAAV | 1080 | WO2016081811A1 SEQ ID NO: 84 |
| rAAV | 1081 | WO2016081811A1 SEQ ID NO: 85 |
| rAAV | 1082 | WO2016081811A1 SEQ ID NO: 86 |
| rAAV | 1083 | WO2016081811A1 SEQ ID NO: 87 |
| rAAV | 1084 | WO2016081811A1 SEQ ID NO: 88 |
| rAAV | 1085 | WO2016081811A1 SEQ ID NO: 89 |
| rAAV | 1086 | WO2016081811A1 SEQ ID NO: 90 |
| rAAV | 1087 | WO2016081811A1 SEQ ID NO: 91 |
| rAAV | 1088 | WO2016081811A1 SEQ ID NO: 92 |
| rAAV | 1089 | WO2016081811A1 SEQ ID NO: 93 |
| rAAV | 1090 | WO2016081811A1 SEQ ID NO: 94 |
| rAAV | 1091 | WO2016081811A1 SEQ ID NO: 95 |
| rAAV | 1092 | WO2016081811A1 SEQ ID NO: 96 |
| rAAV | 1093 | WO2016081811A1 SEQ ID NO: 97 |
| rAAV | 1094 | WO2016081811A1 SEQ ID NO: 98 |
| rAAV | 1095 | WO2016081811A1 SEQ ID NO: 99 |
| rAAV | 1096 | WO2016081811A1 SEQ ID NO: 100 |
| rAAV | 1097 | WO2016081811A1 SEQ ID NO: 101 |
| rAAV | 1098 | WO2016081811A1 SEQ ID NO: 102 |
| rAAV | 1099 | WO2016081811A1 SEQ ID NO: 103 |
| rAAV | 1100 | WO2016081811A1 SEQ ID NO: 104 |
| rAAV | 1101 | WO2016081811A1 SEQ ID NO: 105 |
| rAAV | 1102 | WO2016081811A1 SEQ ID NO: 106 |
| rAAV | 1103 | WO2016081811A1 SEQ ID NO: 107 |
| rAAV | 1104 | WO2016081811A1 SEQ ID NO: 108 |
| rAAV | 1105 | WO2016081811A1 SEQ ID NO: 109 |
| rAAV | 1106 | WO2016081811A1 SEQ ID NO: 110 |
| rAAV | 1107 | WO2016081811A1 SEQ ID NO: 111 |
| rAAV | 1108 | WO2016081811A1 SEQ ID NO: 112 |
| rAAV | 1109 | WO2016081811A1 SEQ ID NO: 113 |
| rAAV | 1110 | WO2016081811A1 SEQ ID NO: 114 |
| rAAV | 1111 | WO2016081811A1 SEQ ID NO: 115 |
| rAAV | 1112 | WO2016081811A1 SEQ ID NO: 116 |
| rAAV | 1113 | WO2016081811A1 SEQ ID NO: 117 |
| rAAV | 1114 | WO2016081811A1 SEQ ID NO: 118 |
| rAAV | 1115 | WO2016081811A1 SEQ ID NO: 119 |
| rAAV | 1116 | WO2016081811A1 SEQ ID NO: 120 |
| rAAV | 1117 | WO2016081811A1 SEQ ID NO: 121 |
| rAAV | 1118 | WO2016081811A1 SEQ ID NO: 122 |
| rAAV | 1119 | WO2016081811A1 SEQ ID NO: 123 |
| rAAV | 1120 | WO2016081811A1 SEQ ID NO: 124 |
| rAAV | 1121 | WO2016081811A1 SEQ ID NO: 125 |
| rAAV | 1122 | WO2016081811A1 SEQ ID NO: 126 |
| rAAV | 1123 | WO2016081811A1 SEQ ID NO: 127 |
| rAAV | 1124 | WO2016081811A1 SEQ ID NO: 128 |
| AAV8 E532K | 1125 | WO2016081811A1 SEQ ID NO: 133 |
| AAV8 E532K | 1126 | WO2016081811A1 SEQ ID NO: 134 |
| rAAV4 | 1127 | WO2016115382A1 SEQ ID NO: 2 |
| rAAV4 | 1128 | WO2016115382A1 SEQ ID NO: 3 |
| rAAV4 | 1129 | WO2016115382A1 SEQ ID NO: 4 |
| rAAV4 | 1130 | WO2016115382A1 SEQ ID NO: 5 |
| rAAV4 | 1131 | WO2016115382A1 SEQ ID NO: 6 |
| rAAV4 | 1132 | WO2016115382A1 SEQ ID NO: 7 |
| rAAV4 | 1133 | WO2016115382A1 SEQ ID NO: 8 |
| rAAV4 | 1134 | WO2016115382A1 SEQ ID NO: 9 |
| rAAV4 | 1135 | WO2016115382A1 SEQ ID NO: 10 |
| rAAV4 | 1136 | WO2016115382A1 SEQ ID NO: 11 |
| rAAV4 | 1137 | WO2016115382A1 SEQ ID NO: 12 |
| rAAV4 | 1138 | WO2016115382A1 SEQ ID NO: 13 |
| rAAV4 | 1139 | WO2016115382A1 SEQ ID NO: 14 |
| rAAV4 | 1140 | WO2016115382A1 SEQ ID NO: 15 |
| rAAV4 | 1141 | WO2016115382A1 SEQ ID NO: 16 |
| rAAV4 | 1142 | WO2016115382A1 SEQ ID NO: 17 |
| rAAV4 | 1143 | WO2016115382A1 SEQ ID NO: 18 |
| rAAV4 | 1144 | WO2016115382A1 SEQ ID NO: 19 |
| rAAV4 | 1145 | WO2016115382A1 SEQ ID NO: 20 |
| rAAV4 | 1146 | WO2016115382A1 SEQ ID NO: 21 |
| AAV11 | 1147 | WO2016115382A1 SEQ ID NO: 22 |
| AAV12 | 1148 | WO2016115382A1 SEQ ID NO: 23 |
| rh32 | 1149 | WO2016115382A1 SEQ ID NO: 25 |
| rh33 | 1150 | WO2016115382A1 SEQ ID NO: 26 |
| rh34 | 1151 | WO2016115382A1 SEQ ID NO: 27 |
| rAAV4 | 1152 | WO2016115382A1 SEQ ID NO: 28 |
| rAAV4 | 1153 | WO2016115382A1 SEQ ID NO: 29 |
| rAAV4 | 1154 | WO2016115382A1 SEQ ID NO: 30 |
| rAAV4 | 1155 | WO2016115382A1 SEQ ID NO: 31 |
| rAAV4 | 1156 | WO2016115382A1 SEQ ID NO: 32 |
| rAAV4 | 1157 | WO2016115382A1 SEQ ID NO: 33 |
| AAV2/8 | 1158 | WO2016131981A1 SEQ ID NO: 47 |
| AAV2/8 | 1159 | WO2016131981A1 SEQ ID NO: 48 |
| ancestral AAV | 1160 | WO2016154344A1 SEQ ID NO: 7 |
| ancestral AAV variant C4 | 1161 | WO2016154344A1 SEQ ID NO: 13 |
| ancestral AAV variant C7 | 1162 | WO2016154344A1 SEQ ID NO: 14 |
| ancestral AAV variant G4 | 1163 | WO2016154344A1 SEQ ID NO: 15 |
| consensus amino acid sequence | 1164 | WO2016154344A1 SEQ ID NO: 16 |
| of ancestral AAV variants, C4, | | |
| C7 and G4 | | |
| consensus amino acid sequence | 1165 | WO2016154344A1 SEQ ID NO: 17 |
| of ancestral AAV variants, C4 | | |
| and C7 | | |
| AAV8 (with a AAV2 | 1166 | WO2016150403A1 SEQ ID NO: 13 |
| phospholipase domain) | | |
| AAV VR-942n | 1167 | US20160289275A1 SEQ ID NO: 10 |
| AAV5-A (M569V) | 1168 | US20160289275A1 SEQ ID NO: 13 |
| AAV5-A (M569V) | 1169 | US20160289275A1 SEQ ID NO: 14 |
| AAV5-A (Y585V) | 1170 | US20160289275A1 SEQ ID NO: 16 |
| AAV5-A (Y585V) | 1171 | US20160289275A1 SEQ ID NO: 17 |
| AAV5-A (L587T) | 1172 | US20160289275A1 SEQ ID NO: 19 |
| AAV5-A (L587T) | 1173 | US20160289275A1 SEQ ID NO: 20 |
| AAV5-A (Y585V/L587T) | 1174 | US20160289275A1 SEQ ID NO: 22 |
| AAV5-A (Y585V7L587T) | 1175 | US20160289275A1 SEQ ID NO: 23 |
| AAV5-B (D652A) | 1176 | US20160289275A1 SEQ ID NO: 25 |
| AAV5-B (D652A) | 1177 | US20160289275A1 SEQ ID NO: 26 |
| AAV5-B (T362M) | 1178 | US20160289275A1 SEQ ID NO: 28 |
| AAV5-B (T362M) | 1179 | US20160289275A1 SEQ ID NO: 29 |
| AAV5-B (Q359D) | 1180 | US20160289275A1 SEQ ID NO: 31 |
| AAV5-B (Q359D) | 1181 | US20160289275A1 SEQ ID NO: 32 |
| AAV5-B (E350Q) | 1182 | US20160289275A1 SEQ ID NO: 34 |
| AAV5-B (E350Q) | 1183 | US20160289275A1 SEQ ID NO: 35 |
| AAV5-B (P533S) | 1184 | US20160289275A1 SEQ ID NO: 37 |
| AAV5-B (P533S) | 1185 | US20160289275A1 SEQ ID NO: 38 |
| AAV5-B (P533G) | 1186 | US20160289275A1 SEQ ID NO: 40 |
| AAV5-B (P533G) | 1187 | US20160289275A1 SEQ ID NO: 41 |
| AAV5-mutation in loop VII | 1188 | US20160289275A1 SEQ ID NO: 43 |
| AAV5 -mutation in loop VII | 1189 | US20160289275A1 SEQ ID NO: 44 |
| AAV8 | 1190 | US20160289275A1 SEQ ID NO: 47 |
| Mut A (LK03/AAV8) | 1191 | WO2016181123A1 SEQ ID NO: 1 |
| Mut B (LK03/AAV5) | 1192 | WO2016181123A1 SEQ ID NO: 2 |
| Mut C (AAV8/AAV3B) | 1193 | WO2016181123A1 SEQ ID NO: 3 |
| Mut D (AAV5/AAV3B) | 1194 | WO2016181123A1 SEQ ID NO: 4 |
| Mut E (AAV8/AAV3B) | 1195 | WO2016181123A1 SEQ ID NO: 5 |
| Mut F (AAV3B/AAV8) | 1196 | WO2016181123A1 SEQ ID NO: 6 |
| AAV44.9 | 1197 | WO2016183297A1 SEQ ID NO: 4 |
| AAV44.9 | 1198 | WO2016183297A1 SEQ ID NO: 5 |
| AAVrh8 | 1199 | WO2016183297A1 SEQ ID NO: 6 |
| AAV44.9 (S470N) | 1200 | WO2016183297A1 SEQ ID NO: 9 |
| rh74 VP1 | 1201 | US20160375110A1 SEQ ID NO: 1 |
| AAV-LK03 (L125I) | 1202 | WO2017015102A1 SEQ ID NO: 5 |
| AAV3B (S663V + T492V) | 1203 | WO2017015102A1 SEQ ID NO: 6 |
| Anc80 | 1204 | WO2017019994A2 SEQ ID NO: 1 |
| Anc80 | 1205 | WO2017019994A2 SEQ ID NO: 2 |
| Anc81 | 1206 | WO2017019994A2 SEQ ID NO: 3 |
| Anc81 | 1207 | WO2017019994A2 SEQ ID NO: 4 |
| Anc82 | 1208 | WO2017019994A2 SEQ ID NO: 5 |
| Anc82 | 1209 | WO2017019994A2 SEQ ID NO: 6 |
| Anc83 | 1210 | WO2017019994A2 SEQ ID NO: 7 |
| Anc83 | 1211 | WO2017019994A2 SEQ ID NO: 8 |
| Anc84 | 1212 | WO2017019994A2 SEQ ID NO: 9 |
| Anc84 | 1213 | WO2017019994A2 SEQ ID NO: 10 |
| Anc94 | 1214 | WO2017019994A2 SEQ ID NO: 11 |
| Anc94 | 1215 | WO2017019994A2 SEQ ID NO: 12 |
| Anc113 | 1216 | WO2017019994A2 SEQ ID NO: 13 |
| Anc13 | 1217 | WO2017019994A2 SEQ ID NO: 14 |
| Anc126 | 1218 | WO2017019994A2 SEQ ID NO: 15 |
| Anc126 | 1219 | WO2017019994A2 SEQ ID NO: 16 |
| Anc127 | 1220 | WO2017019994A2 SEQ ID NO: 17 |
| Anc127 | 1221 | WO2017019994A2 SEQ ID NO: 18 |
| Anc80L27 | 1222 | WO2017019994A2 SEQ ID NO: 19 |
| Anc80L59 | 1223 | WO2017019994A2 SEQ ID NO: 20 |
| Anc80L60 | 1224 | WO2017019994A2 SEQ ID NO: 21 |
| Anc80L62 | 1225 | WO2017019994A2 SEQ ID NO: 22 |
| Anc80L65 | 1226 | WO2017019994A2 SEQ ID NO: 23 |
| Anc80L33 | 1227 | WO2017019994A2 SEQ ID NO: 24 |
| Anc80L36 | 1228 | WO2017019994A2 SEQ ID NO: 25 |
| Anc80L44 | 1229 | WO2017019994A2 SEQ ID NO: 26 |
| Anc80L1 | 1230 | WO2017019994A2 SEQ ID NO: 35 |
| Anc80L1 | 1231 | WO2017019994A2 SEQ ID NO: 36 |
| AAVrh10 | 1232 | WO2017019994A2 SEQ ID NO: 41 |
| Anc110 | 1233 | WO2017019994A2 SEQ ID NO: 42 |
| Anc110 | 1234 | WO2017019994A2 SEQ ID NO: 43 |
| AAVrh32.33 | 1235 | WO2017019994A2 SEQ ID NO: 45 |
| AAVrh74 | 1236 | WO2017049031A1 SEQ ID NO: 1 |
| AAV2 | 1237 | WO2017053629A2 SEQ ID NO: 49 |
| AAV2 | 1238 | WO2017053629A2 SEQ ID NO: 50 |
| AAV2 | 1239 | WO2017053629A2 SEQ ID NO: 82 |
| Parvo-like virus | 1240 | WO2017070476A2 SEQ ID NO: 1 |
| Parvo-like virus | 1241 | WO2017070476A2 SEQ ID NO: 2 |
| Parvo-like virus | 1242 | WO2017070476A2 SEQ ID NO: 3 |
| Parvo-like virus | 1243 | WO2017070476A2 SEQ ID NO: 4 |
| Parvo-like virus | 1244 | WO2017070476A2 SEQ ID NO: 5 |
| Parvo-like virus | 1245 | WO2017070476A2 SEQ ID NO: 6 |
| AAVrh.10 | 1246 | WO2017070516A1 SEQ ID NO: 7 |
| AAVrh.10 | 1247 | WO2017070516A1 SEQ ID NO: 14 |
| AAV2tYF | 1248 | WO2017070491A1 SEQ ID NO: 1 |
| AAV-SPK | 1249 | WO2017075619A1 SEQ ID NO: 28 |
| AAV2.5 | 1250 | US20170128528A1 SEQ ID NO: 13 |
| AAV1.1 | 1251 | US20170128528A1 SEQ ID NO: 15 |
| AAV6.1 | 1252 | US20170128528A1 SEQ ID NO: 17 |
| AAV6.3.1 | 1253 | US20170128528A1 SEQ ID NO: 18 |
| AAV2i8 | 1254 | US20170128528A1 SEQ ID NO: 28 |
| AAV2i8 | 1255 | US20170128528A1 SEQ ID NO: 29 |
| ttAAV | 1256 | US20170128528A1 SEQ ID NO: 30 |
| ttAAV-S312N | 1257 | US20170128528A1 SEQ ID NO: 32 |
| ttAAV-S312N | 1258 | US20170128528A1 SEQ ID NO: 33 |
| AAV6 (Y705, Y731, and T492) | 1259 | WO2016134337A1 SEQ ID NO: 24 |
| AAV2 | 1260 | WO2016134375A1 SEQ ID NO: 9 |
| AAV2 | 1261 | WO2016134375A1 SEQ ID NO: 10 |
| AAV2 variant | 2322 | WO2018071831 SEQ ID NO: 1 |
| AAV2 variant | 2323 | WO2018071831 SEQ ID NO: 2 |
| AAV2 variant | 2324 | WO2018071831 SEQ ID NO: 3 |
| AAV2 variant | 2325 | WO2018071831 SEQ ID NO: 4 |
| AAV2 variant | 2326 | WO2018071831 SEQ ID NO: 5 |
| AAV2 variant | 2327 | WO2018071831 SEQ ID NO: 6 |
| AAV2 variant | 2328 | WO2018071831 SEQ ID NO: 7 |
| AAV2 variant | 2329 | WO2018071831 SEQ ID NO: 8 |
| AAV2 variant | 2330 | WO2018071831 SEQ ID NO: 9 |
| AAV2 variant | 2331 | WO2018071831 SEQ ID NO: 10 |
| AAV2 variant | 2332 | WO2018071831 SEQ ID NO: 11 |
| AAV2 variant | 2333 | WO2018071831 SEQ ID NO: 12 |
| AAV2 variant | 2334 | WO2018071831 SEQ ID NO: 13 |
| AAV2 variant | 2335 | WO2018071831 SEQ ID NO: 14 |
| AAV2 variant | 2336 | WO2018071831 SEQ ID NO: 15 |
| AAV2 variant | 2337 | WO2018071831 SEQ ID NO: 16 |
| AAV2 variant | 2338 | WO2018071831 SEQ ID NO: 17 |
| AAV2 variant | 2339 | WO2018071831 SEQ ID NO: 18 |
| AAV2 variant | 2340 | WO2018071831 SEQ ID NO: 19 |
| AAV2 variant | 2341 | WO2018071831 SEQ ID NO: 20 |
| AAV2 variant | 2342 | WO2018071831 SEQ ID NO: 21 |
| AAV2 variant | 2343 | WO2018071831 SEQ ID NO: 22 |
| AAV2 variant | 2344 | WO2018071831 SEQ ID NO: 23 |
| AAV2 variant | 2345 | WO2018071831 SEQ ID NO: 24 |
| AAV2 variant | 2346 | WO2018071831 SEQ ID NO: 25 |
| AAV2 variant | 2347 | WO2018071831 SEQ ID NO: 26 |
| AAV2 variant | 2348 | WO2018071831 SEQ ID NO: 27 |
| AAV2 variant | 2349 | WO2018071831 SEQ ID NO: 28 |
| AAV2 variant | 2350 | WO2018071831 SEQ ID NO: 29 |
| AAV2 variant | 2351 | WO2018071831 SEQ ID NO: 30 |
| AAV2 variant | 2352 | WO2018071831 SEQ ID NO: 31 |
| AAV2 variant | 2353 | WO2018071831 SEQ ID NO: 32 |
| AAV2 variant | 2354 | WO2018071831 SEQ ID NO: 33 |
| AAV2 variant | 2355 | WO2018071831 SEQ ID NO: 34 |
| AAV2 variant | 2356 | WO2018071831 SEQ ID NO: 35 |
| AAV2 variant | 2357 | WO2018071831 SEQ ID NO: 36 |
| AAV2 variant | 2358 | WO2018071831 SEQ ID NO: 37 |
| AAV2 variant | 2359 | WO2018071831 SEQ ID NO: 38 |
| AAV2 variant | 2360 | WO2018071831 SEQ ID NO: 39 |
| AAV2 variant | 2361 | WO2018071831 SEQ ID NO: 40 |
| AAV2 variant | 2362 | WO2018071831 SEQ ID NO: 41 |
| AAV2 variant | 2363 | WO2018071831 SEQ ID NO: 42 |
| AAV2 variant | 2364 | WO2018071831 SEQ ID NO: 43 |
| AAV2 variant | 2365 | WO2018071831 SEQ ID NO: 44 |
| AAV2 variant | 2366 | WO2018071831 SEQ ID NO: 45 |
| AAV2 variant | 2367 | WO2018071831 SEQ ID NO: 46 |
| AAV2 variant | 2368 | WO2018071831 SEQ ID NO: 47 |
| AAV2 variant | 2369 | WO2018071831 SEQ ID NO: 48 |
| AAV2 variant | 2370 | WO2018071831 SEQ ID NO: 49 |
| AAV2 variant | 2371 | WO2018071831 SEQ ID NO: 50 |
| AAV2 variant | 2372 | WO2018071831 SEQ ID NO: 51 |
| AAV2 variant | 2373 | WO2018071831 SEQ ID NO: 52 |
| AAV2 variant | 2374 | WO2018071831 SEQ ID NO: 53 |
| AAV2 variant | 2375 | WO2018071831 SEQ ID NO: 54 |
| AAV2 variant | 2376 | WO2018071831 SEQ ID NO: 55 |
| AAV2 variant | 2377 | WO2018071831 SEQ ID NO: 56 |
| AAV2 variant | 2378 | WO2018071831 SEQ ID NO: 57 |
| AAV2 variant | 2379 | WO2018071831 SEQ ID NO: 58 |
| AAV2 variant | 2380 | WO2018071831 SEQ ID NO: 59 |
| AAV2 variant | 2381 | WO2018071831 SEQ ID NO: 60 |
| AAV2 variant | 2382 | WO2018071831 SEQ ID NO: 61 |
| AAV2 variant | 2383 | WO2018071831 SEQ ID NO: 62 |
| AAV2 variant | 2384 | WO2018071831 SEQ ID NO: 63 |
| AAV2 variant | 2385 | WO2018071831 SEQ ID NO: 64 |
| AAV2 variant | 2386 | WO2018071831 SEQ ID NO: 65 |
| AAV2 variant | 2387 | WO2018071831 SEQ ID NO: 66 |
| AAV2 variant | 2388 | WO2018071831 SEQ ID NO: 67 |
| AAV2 variant | 2389 | WO2018071831 SEQ ID NO: 68 |
| AAV2 variant | 2390 | WO2018071831 SEQ ID NO: 69 |
| AAV2 variant | 2391 | WO2018071831 SEQ ID NO: 70 |
| AAV2 variant | 2392 | WO2018071831 SEQ ID NO: 71 |
| AAV2 variant | 2393 | WO2018071831 SEQ ID NO: 72 |
| AAV2 variant | 2394 | WO2018071831 SEQ ID NO: 73 |
| AAV2 variant | 2395 | WO2018071831 SEQ ID NO: 74 |
| AAV2 variant | 2396 | WO2018071831 SEQ ID NO: 75 |
| AAV2 variant | 2397 | WO2018071831 SEQ ID NO: 76 |
| AAV2 variant | 2398 | WO2018071831 SEQ ID NO: 77 |
| AAV2 variant | 2399 | WO2018071831 SEQ ID NO: 78 |
| AAV2 variant | 2400 | WO2018071831 SEQ ID NO: 79 |
| AAV2 variant | 2401 | WO2018071831 SEQ ID NO: 80 |
| AAV2 variant | 2402 | WO2018071831 SEQ ID NO: 81 |
| AAV2 variant | 2403 | WO2018071831 SEQ ID NO: 82 |
| AAV2 variant | 2404 | WO2018071831 SEQ ID NO: 83 |
| AAV2 variant | 2405 | WO2018071831 SEQ ID NO: 84 |
| AAV2 variant | 2406 | WO2018071831 SEQ ID NO: 85 |
| AAV2 variant | 2407 | WO2018071831 SEQ ID NO: 86 |
| AAV2 variant | 2408 | WO2018071831 SEQ ID NO: 87 |
| AAV2 variant | 2409 | WO2018071831 SEQ ID NO: 88 |
| AAV2 variant | 2410 | WO2018071831 SEQ ID NO: 89 |
| AAV2 variant | 2411 | WO2018071831 SEQ ID NO: 90 |
| AAV2 variant | 2412 | WO2018071831 SEQ ID NO: 91 |
| AAV2 variant | 2413 | WO2018071831 SEQ ID NO: 92 |
| AAV2 variant | 2414 | WO2018071831 SEQ ID NO: 93 |
| AAV2 variant | 2415 | WO2018071831 SEQ ID NO: 94 |
| AAV2 variant | 2416 | WO2018071831 SEQ ID NO: 95 |
| AAV2 variant | 2417 | WO2018071831 SEQ ID NO: 96 |
| AAV2 variant | 2418 | WO2018071831 SEQ ID NO: 97 |
| AAV2 variant | 2419 | WO2018071831 SEQ ID NO: 98 |
| AAV2 variant | 2420 | WO2018071831 SEQ ID NO: 99 |
| AAV2 variant | 2421 | WO2018071831 SEQ ID NO: 100 |
| AAV2 variant | 2422 | WO2018071831 SEQ ID NO: 101 |
| AAV2 variant | 2423 | WO2018071831 SEQ ID NO: 102 |
| AAV2 variant | 2424 | WO2018071831 SEQ ID NO: 103 |
| AAV2 variant | 2425 | WO2018071831 SEQ ID NO: 104 |
| AAV2 variant | 2426 | WO2018071831 SEQ ID NO: 105 |
| AAV2 variant | 2427 | WO2018071831 SEQ ID NO: 106 |
| AAV2 variant | 2428 | WO2018071831 SEQ ID NO: 107 |
| AAV2 variant | 2429 | WO2018071831 SEQ ID NO: 108 |
| AAV2 variant | 2430 | WO2018071831 SEQ ID NO: 109 |
| AAV2 variant | 2431 | WO2018071831 SEQ ID NO: 110 |
| AAV2 variant | 2432 | WO2018071831 SEQ ID NO: 111 |
| AAV2 variant | 2433 | WO2018071831 SEQ ID NO: 112 |
| AAV2 variant | 2434 | WO2018071831 SEQ ID NO: 113 |
| AAV2 variant | 2435 | WO2018071831 SEQ ID NO: 114 |
| AAV2 variant | 2436 | WO2018071831 SEQ ID NO: 115 |
| AAV2 variant | 2437 | WO2018071831 SEQ ID NO: 116 |
| AAV2 variant | 2438 | WO2018071831 SEQ ID NO: 117 |
| AAV2 variant | 2439 | WO2018071831 SEQ ID NO: 118 |
| AAV2 variant | 2440 | WO2018071831 SEQ ID NO: 119 |
| AAV2 variant | 2441 | WO2018071831 SEQ ID NO: 120 |
| AAV2 variant | 2442 | WO2018071831 SEQ ID NO: 121 |
| AAV2 variant | 2443 | WO2018071831 SEQ ID NO: 122 |
| AAV2 variant | 2444 | WO2018071831 SEQ ID NO: 123 |
| AAV2 variant | 2445 | WO2018071831 SEQ ID NO: 124 |
| AAV2 variant | 2446 | WO2018071831 SEQ ID NO: 125 |
| AAV2 variant | 2447 | WO2018071831 SEQ ID NO: 126 |
| AAV2 variant | 2448 | WO2018071831 SEQ ID NO: 127 |
| AAV2 variant | 2449 | WO2018071831 SEQ ID NO: 128 |
| AAV2 variant | 2450 | WO2018071831 SEQ ID NO: 129 |
| AAV2 variant | 2451 | WO2018071831 SEQ ID NO: 130 |
| AAV2 variant | 2452 | WO2018071831 SEQ ID NO: 131 |
| AAV2 variant | 2453 | WO2018071831 SEQ ID NO: 132 |
| AAV2 variant | 2454 | WO2018071831 SEQ ID NO: 133 |
| AAV2 variant | 2455 | WO2018071831 SEQ ID NO: 134 |
| AAV2 variant | 2456 | WO2018071831 SEQ ID NO: 135 |
| AAV2 variant | 2457 | WO2018071831 SEQ ID NO: 136 |
| AAV2 variant | 2458 | WO2018071831 SEQ ID NO: 137 |
| AAV2 variant | 2459 | WO2018071831 SEQ ID NO: 138 |
| AAV2 variant | 2460 | WO2018071831 SEQ ID NO: 139 |
| AAV2 variant | 2461 | WO2018071831 SEQ ID NO: 140 |
| AAV2 variant | 2462 | WO2018071831 SEQ ID NO: 141 |
| AAV2 variant | 2463 | WO2018071831 SEQ ID NO: 142 |
| AAV2 variant | 2464 | WO2018071831 SEQ ID NO: 143 |
| AAV2 variant | 2465 | WO2018071831 SEQ ID NO: 144 |
| AAV2 variant | 2466 | WO2018071831 SEQ ID NO: 145 |
| AAV2 variant | 2467 | WO2018071831 SEQ ID NO: 146 |
| AAV2 variant | 2468 | WO2018071831 SEQ ID NO: 147 |
| AAV2 variant | 2469 | WO2018071831 SEQ ID NO: 148 |
| AAV2 variant | 2470 | WO2018071831 SEQ ID NO: 149 |
| AAV2 variant | 2471 | WO2018071831 SEQ ID NO: 150 |
| AAV2 variant | 2472 | WO2018071831 SEQ ID NO: 151 |
| AAV2 variant | 2473 | WO2018071831 SEQ ID NO: 152 |
| AAV2 variant | 2474 | WO2018071831 SEQ ID NO: 153 |
| AAV2 variant | 2475 | WO2018071831 SEQ ID NO: 154 |
| AAV2 variant | 2476 | WO2018071831 SEQ ID NO: 155 |
| AAV2 variant | 2477 | WO2018071831 SEQ ID NO: 156 |
| AAV2 variant | 2478 | WO2018071831 SEQ ID NO: 157 |
| AAV2 variant | 2479 | WO2018071831 SEQ ID NO: 158 |
| AAV2 variant | 2480 | WO2018071831 SEQ ID NO: 159 |
| AAV2 variant | 2481 | WO2018071831 SEQ ID NO: 160 |
| AAV2 variant | 2482 | WO2018071831 SEQ ID NO: 161 |
| AAV2 variant | 2483 | WO2018071831 SEQ ID NO: 162 |
| AAV2 variant | 2484 | WO2018071831 SEQ ID NO: 163 |
| AAV2 variant | 2485 | WO2018071831 SEQ ID NO: 164 |
| AAV2 variant | 2486 | WO2018071831 SEQ ID NO: 165 |
| AAV2 variant | 2487 | WO2018071831 SEQ ID NO: 166 |
| AAV2 variant | 2488 | WO2018071831 SEQ ID NO: 167 |
| AAV2 variant | 2489 | WO2018071831 SEQ ID NO: 168 |
| AAV2 variant | 2490 | WO2018071831 SEQ ID NO: 169 |
| AAV2 variant | 2491 | WO2018071831 SEQ ID NO: 170 |
| AAV2 variant | 2492 | WO2018071831 SEQ ID NO: 171 |
| AAV2 variant | 2493 | WO2018071831 SEQ ID NO: 172 |
| AAV2 variant | 2494 | WO2018071831 SEQ ID NO: 173 |
| AAV2 variant | 2495 | WO2018071831 SEQ ID NO: 174 |
| AAV2 variant | 2496 | WO2018071831 SEQ ID NO: 175 |
| AAV2 variant | 2497 | WO2018071831 SEQ ID NO: 176 |
| AAV2 variant | 2498 | WO2018071831 SEQ ID NO: 177 |
| AAV2 variant | 2499 | WO2018071831 SEQ ID NO: 178 |
| AAV2 variant | 2500 | WO2018071831 SEQ ID NO: 179 |
| AAV2 variant | 2501 | WO2018071831 SEQ ID NO: 180 |
| AAV2 variant | 2502 | WO2018071831 SEQ ID NO: 181 |
| AAV2 variant | 2503 | WO2018071831 SEQ ID NO: 182 |
| AAV2 variant | 2504 | WO2018071831 SEQ ID NO: 183 |
| AAV2 variant | 2505 | WO2018071831 SEQ ID NO: 184 |
| AAV2 variant | 2506 | WO2018071831 SEQ ID NO: 185 |
| AAV2 variant | 2507 | WO2018071831 SEQ ID NO: 186 |
| AAV2 variant | 2508 | WO2018071831 SEQ ID NO: 187 |
| AAV2 variant | 2509 | WO2018071831 SEQ ID NO: 188 |
| AAV2 variant | 2510 | WO2018071831 SEQ ID NO: 189 |
| AAV2 variant | 2511 | WO2018071831 SEQ ID NO: 190 |
| AAV2 variant | 2512 | WO2018071831 SEQ ID NO: 191 |
| AAV2 variant | 2513 | WO2018071831 SEQ ID NO: 192 |
| AAV2 variant | 2514 | WO2018071831 SEQ ID NO: 193 |
| AAV2 variant | 2515 | WO2018071831 SEQ ID NO: 194 |
| AAV2 variant | 2516 | WO2018071831 SEQ ID NO: 195 |
| AAV2 variant | 2517 | WO2018071831 SEQ ID NO: 196 |
| AAV2 variant | 2518 | WO2018071831 SEQ ID NO: 197 |
| AAV2 variant | 2519 | WO2018071831 SEQ ID NO: 198 |
| AAV2 variant | 2520 | WO2018071831 SEQ ID NO: 199 |
| AAV2 variant | 2521 | WO2018071831 SEQ ID NO: 200 |
| AAV2 variant | 2522 | WO2018071831 SEQ ID NO: 201 |
| AAV2 variant | 2523 | WO2018071831 SEQ ID NO: 202 |
| AAV2 variant | 2524 | WO2018071831 SEQ ID NO: 203 |
| AAV2 variant | 2525 | WO2018071831 SEQ ID NO: 204 |
| AAV2 variant | 2526 | WO2018071831 SEQ ID NO: 205 |
| AAV2 variant | 2527 | WO2018071831 SEQ ID NO: 206 |
| AAV2 variant | 2528 | WO2018071831 SEQ ID NO: 207 |
| AAV2 variant | 2529 | WO2018071831 SEQ ID NO: 208 |
| AAV2 variant | 2530 | WO2018071831 SEQ ID NO: 209 |
| AAV2 variant | 2531 | WO2018071831 SEQ ID NO: 210 |
| AAV2 variant | 2532 | WO2018071831 SEQ ID NO: 211 |
| AAV2 variant | 2533 | WO2018071831 SEQ ID NO: 212 |
| AAV2 variant | 2534 | WO2018071831 SEQ ID NO: 213 |
| AAV2 variant | 2535 | WO2018071831 SEQ ID NO: 214 |
| AAV2 variant | 2536 | WO2018071831 SEQ ID NO: 215 |
| AAV2 variant | 2537 | WO2018071831 SEQ ID NO: 216 |
| AAV2 variant | 2538 | WO2018071831 SEQ ID NO: 217 |
| AAV2 variant | 2539 | WO2018071831 SEQ ID NO: 218 |
| AAV2 variant | 2540 | WO2018071831 SEQ ID NO: 219 |
| AAV2 variant | 2541 | WO2018071831 SEQ ID NO: 220 |
| AAV2 variant | 2542 | WO2018071831 SEQ ID NO: 221 |
| AAV2 variant | 2543 | WO2018071831 SEQ ID NO: 222 |
| AAV2 variant | 2544 | WO2018071831 SEQ ID NO: 223 |
| AAV2 variant | 2545 | WO2018071831 SEQ ID NO: 224 |
| AAV2 variant | 2546 | WO2018071831 SEQ ID NO: 225 |
| AAV2 variant | 2547 | WO2018071831 SEQ ID NO: 226 |
| AAV2 variant | 2548 | WO2018071831 SEQ ID NO: 227 |
| AAV2 variant | 2549 | WO2018071831 SEQ ID NO: 228 |
| AAV2 variant | 2550 | WO2018071831 SEQ ID NO: 229 |
| AAV2 variant | 2551 | WO2018071831 SEQ ID NO: 230 |
| AAV2 variant | 2552 | WO2018071831 SEQ ID NO: 231 |
| AAV2 variant | 2553 | WO2018071831 SEQ ID NO: 232 |
| AAV2 variant | 2554 | WO2018071831 SEQ ID NO: 233 |
| AAV2 variant | 2555 | WO2018071831 SEQ ID NO: 234 |
| AAV2 variant | 2556 | WO2018071831 SEQ ID NO: 235 |
| AAV2 variant | 2557 | WO2018071831 SEQ ID NO: 236 |
| AAV2 variant | 2558 | WO2018071831 SEQ ID NO: 237 |
| AAV2 variant | 2559 | WO2018071831 SEQ ID NO: 238 |
| AAV2 variant | 2560 | WO2018071831 SEQ ID NO: 239 |
| AAV2 variant | 2561 | WO2018071831 SEQ ID NO: 240 |
| AAV2 variant | 2562 | WO2018071831 SEQ ID NO: 241 |
| AAV2 variant | 2563 | WO2018071831 SEQ ID NO: 242 |
| AAV2 variant | 2564 | WO2018071831 SEQ ID NO: 243 |
| AAV2 variant | 2565 | WO2018071831 SEQ ID NO: 244 |
| AAV2 variant | 2566 | WO2018071831 SEQ ID NO: 245 |
| AAV2 variant | 2567 | WO2018071831 SEQ ID NO: 246 |
| AAV2 variant | 2568 | WO2018071831 SEQ ID NO: 247 |
| AAV2 variant | 2569 | WO2018071831 SEQ ID NO: 248 |
| AAV2 variant | 2570 | WO2018071831 SEQ ID NO: 249 |
| AAV2 variant | 2571 | WO2018071831 SEQ ID NO: 250 |
| AAV2 variant | 2572 | WO2018071831 SEQ ID NO: 251 |
| AAV2 variant | 2573 | WO2018071831 SEQ ID NO: 252 |
| AAV2 variant | 2574 | WO2018071831 SEQ ID NO: 253 |
| AAV2 variant | 2575 | WO2018071831 SEQ ID NO: 254 |
| AAV2 variant | 2576 | WO2018071831 SEQ ID NO: 255 |
| AAV2 variant | 2577 | WO2018071831 SEQ ID NO: 256 |
| AAV2 variant | 2578 | WO2018071831 SEQ ID NO: 257 |
| AAV2 variant | 2579 | WO2018071831 SEQ ID NO: 258 |
| AAV2 variant | 2580 | WO2018071831 SEQ ID NO: 259 |
| AAV2 variant | 2584 | WO2018071831 SEQ ID NO: 260 |
| AAV2 variant | 2582 | WO2018071831 SEQ ID NO: 261 |
| AAV2 variant | 2583 | WO2018071831 SEQ ID NO: 262 |
| AAV2 variant | 2584 | WO2018071831 SEQ ID NO: 263 |
| AAV2 variant | 2585 | WO2018071831 SEQ ID NO: 264 |
| AAV2 variant | 2586 | WO2018071831 SEQ ID NO: 265 |
| AAV2 variant | 2587 | WO2018071831 SEQ ID NO: 266 |
| AAV2 variant | 2588 | WO2018071831 SEQ ID NO: 267 |
| AAV2 variant | 2589 | WO2018071831 SEQ ID NO: 268 |
| AAV2 variant | 2590 | WO2018071831 SEQ ID NO: 269 |
| AAV2 variant | 2591 | WO2018071831 SEQ ID NO: 270 |
| AAV2 variant | 2592 | WO2018071831 SEQ ID NO: 271 |
| AAV2 variant | 2593 | WO2018071831 SEQ ID NO: 272 |
| AAV2 variant | 2594 | WO2018071831 SEQ ID NO: 273 |
| AAV2 variant | 2595 | WO2018071831 SEQ ID NO: 274 |
| AAV2 variant | 2596 | WO2018071831 SEQ ID NO: 275 |
| AAV2 variant | 2597 | WO2018071831 SEQ ID NO: 276 |
| AAV2 variant | 2598 | WO2018071831 SEQ ID NO: 277 |
| AAV2 variant | 2599 | WO2018071831 SEQ ID NO: 278 |
| AAV2 variant | 2600 | WO2018071831 SEQ ID NO: 279 |
| AAV2 variant | 2601 | WO2018071831 SEQ ID NO: 280 |
| AAV2 variant | 2602 | WO2018071831 SEQ ID NO: 281 |
| AAV2 variant | 2603 | WO2018071831 SEQ ID NO: 282 |
| AAV2 variant | 2604 | WO2018071831 SEQ ID NO: 283 |
| AAV2 variant | 2605 | WO2018071831 SEQ ID NO: 284 |
| AAV2 variant | 2606 | WO2018071831 SEQ ID NO: 285 |
| AAV2 variant | 2607 | WO2018071831 SEQ ID NO: 286 |
| AAV2 variant | 2608 | WO2018071831 SEQ ID NO: 287 |
| AAV2 variant | 2609 | WO2018071831 SEQ ID NO: 288 |
| AAV2 variant | 2610 | WO2018071831 SEQ ID NO: 289 |
| AAV2 variant | 2611 | WO2018071831 SEQ ID NO: 290 |
| AAV2 variant | 2612 | WO2018071831 SEQ ID NO: 291 |
| AAV2 variant | 2613 | WO2018071831 SEQ ID NO: 292 |
| AAV2 variant | 2614 | WO2018071831 SEQ ID NO: 293 |
| AAV2 variant | 2615 | WO2018071831 SEQ ID NO: 294 |
| AAV2 variant | 2616 | WO2018071831 SEQ ID NO: 295 |
| AAV2 variant | 2617 | WO2018071831 SEQ ID NO: 296 |
| AAV2 variant | 2618 | WO2018071831 SEQ ID NO: 297 |
| AAV2 variant | 2619 | WO2018071831 SEQ ID NO: 298 |
| AAV2 variant | 2620 | WO2018071831 SEQ ID NO: 299 |
| AAV2 variant | 2621 | WO2018071831 SEQ ID NO: 300 |
| AAV2 variant | 2622 | WO2018071831 SEQ ID NO: 301 |
| AAV2 variant | 2623 | WO2018071831 SEQ ID NO: 302 |
| AAV2 variant | 2624 | WO2018071831 SEQ ID NO: 303 |
| AAV2 variant | 2625 | WO2018071831 SEQ ID NO: 304 |
| AAV2 variant | 2626 | WO2018071831 SEQ ID NO: 305 |
| AAV2 variant | 2627 | WO2018071831 SEQ ID NO: 306 |
| AAV2 variant | 2628 | WO2018071831 SEQ ID NO: 307 |
| AAV2 variant | 2629 | WO2018071831 SEQ ID NO: 308 |
| AAV2 variant | 2630 | WO2018071831 SEQ ID NO: 309 |
| AAV2 variant | 2631 | WO2018071831 SEQ ID NO: 310 |
| AAV2 variant | 2632 | WO2018071831 SEQ ID NO: 311 |
| AAV2 variant | 2633 | WO2018071831 SEQ ID NO: 312 |
| AAV2 variant | 2634 | WO2018071831 SEQ ID NO: 313 |
| AAV2 variant | 2635 | WO2018071831 SEQ ID NO: 314 |
| AAV2 variant | 2636 | WO2018071831 SEQ ID NO: 315 |
| AAV2 variant | 2637 | WO2018071831 SEQ ID NO: 316 |
| AAV2 variant | 2638 | WO2018071831 SEQ ID NO: 317 |
| AAV2 variant | 2639 | WO2018071831 SEQ ID NO: 318 |
| AAV2 variant | 2640 | WO2018071831 SEQ ID NO: 319 |
| AAV2 variant | 2641 | WO2018071831 SEQ ID NO: 320 |
| AAV2 variant | 2642 | WO2018071831 SEQ ID NO: 321 |
| AAV2 variant | 2643 | WO2018071831 SEQ ID NO: 322 |
| AAV2 variant | 2644 | WO2018071831 SEQ ID NO: 323 |
| AAV2 variant | 2645 | WO2018071831 SEQ ID NO: 324 |
| AAV2 variant | 2646 | WO2018071831 SEQ ID NO: 325 |
| AAV2 variant | 2647 | WO2018071831 SEQ ID NO: 326 |
| AAV2 variant | 2648 | WO2018071831 SEQ ID NO: 327 |
| AAV2 variant | 2649 | WO2018071831 SEQ ID NO: 328 |
| AAV2 variant | 2650 | WO2018071831 SEQ ID NO: 329 |
| AAV2 variant | 2651 | WO2018071831 SEQ ID NO: 330 |
| AAV2 variant | 2652 | WO2018071831 SEQ ID NO: 331 |
| AAV2 variant | 2653 | WO2018071831 SEQ ID NO: 332 |
| AAV2 variant | 2654 | WO2018071831 SEQ ID NO: 333 |
| AAV2 variant | 2655 | WO2018071831 SEQ ID NO: 334 |
| AAV2 variant | 2656 | WO2018071831 SEQ ID NO: 335 |
| AAV2 variant | 2657 | WO2018071831 SEQ ID NO: 336 |
| AAV2 variant | 2658 | WO2018071831 SEQ ID NO: 337 |
| AAV2 variant | 2659 | WO2018071831 SEQ ID NO: 338 |
| AAV2 variant | 2660 | WO2018071831 SEQ ID NO: 339 |
| AAV2 variant | 2661 | WO2018071831 SEQ ID NO: 340 |
| AAV2 variant | 2662 | WO2018071831 SEQ ID NO: 341 |
| AAV2 variant | 2663 | WO2018071831 SEQ ID NO: 342 |
| AAV2 variant | 2664 | WO2018071831 SEQ ID NO: 343 |
| AAV2 variant | 2665 | WO2018071831 SEQ ID NO: 344 |
| AAV2 variant | 2666 | WO2018071831 SEQ ID NO: 345 |
| AAV2 variant | 2667 | WO2018071831 SEQ ID NO: 346 |
| AAV2 variant | 2668 | WO2018071831 SEQ ID NO: 347 |
| AAV2 variant | 2669 | WO2018071831 SEQ ID NO: 348 |
| AAV2 variant | 2670 | WO2018071831 SEQ ID NO: 349 |
| AAV2 variant | 2671 | WO2018071831 SEQ ID NO: 350 |
| AAV2 variant | 2672 | WO2018071831 SEQ ID NO: 351 |
| AAV2 variant | 2673 | WO2018071831 SEQ ID NO: 352 |
| AAV2 variant | 2674 | WO2018071831 SEQ ID NO: 353 |
| AAV2 variant | 2675 | WO2018071831 SEQ ID NO: 354 |
| AAV2 variant | 2676 | WO2018071831 SEQ ID NO: 355 |
| AAV2 variant | 2677 | WO2018071831 SEQ ID NO: 356 |
| AAV2 variant | 2678 | WO2018071831 SEQ ID NO: 357 |
| AAV2 variant | 2679 | WO2018071831 SEQ ID NO: 358 |
| AAV2 variant | 2680 | WO2018071831 SEQ ID NO: 359 |
| AAV2 variant | 2681 | WO2018071831 SEQ ID NO: 360 |
| AAV2 variant | 2682 | WO2018071831 SEQ ID NO: 361 |
| AAV2 variant | 2683 | WO2018071831 SEQ ID NO: 362 |
| AAV2 variant | 2684 | WO2018071831 SEQ ID NO: 363 |
| AAV2 variant | 2685 | WO2018071831 SEQ ID NO: 364 |
| AAV2 variant | 2686 | WO2018071831 SEQ ID NO: 365 |
| AAV2 variant | 2687 | WO2018071831 SEQ ID NO: 366 |
| AAV2 variant | 2688 | WO2018071831 SEQ ID NO: 367 |
| AAV2 variant | 2689 | WO2018071831 SEQ ID NO: 368 |
| AAV2 variant | 2690 | WO2018071831 SEQ ID NO: 369 |
| AAV2 variant | 2691 | WO2018071831 SEQ ID NO: 370 |
| AAV2 variant | 2692 | WO2018071831 SEQ ID NO: 371 |
| AAV2 variant | 2693 | WO2018071831 SEQ ID NO: 372 |
| AAV2 variant | 2694 | WO2018071831 SEQ ID NO: 373 |
| AAV2 variant | 2695 | WO2018071831 SEQ ID NO: 374 |
| AAV2 variant | 2696 | WO2018071831 SEQ ID NO: 375 |
| AAV2 variant | 2697 | WO2018071831 SEQ ID NO: 376 |
| AAV2 variant | 2698 | WO2018071831 SEQ ID NO: 377 |
| AAV2 variant | 2699 | WO2018071831 SEQ ID NO: 378 |
| AAV2 variant | 2700 | WO2018071831 SEQ ID NO: 379 |
| AAV2 variant | 2701 | WO2018071831 SEQ ID NO: 380 |
| AAV2 variant | 2702 | WO2018071831 SEQ ID NO: 381 |
| AAV2 variant | 2703 | WO2018071831 SEQ ID NO: 382 |
| AAV2 variant | 2704 | WO2018071831 SEQ ID NO: 383 |
| AAV2 variant | 2705 | WO2018071831 SEQ ID NO: 384 |
| AAV2 variant | 2706 | WO2018071831 SEQ ID NO: 385 |
| AAV2 variant | 2707 | WO2018071831 SEQ ID NO: 386 |
| AAV2 variant | 2708 | WO2018071831 SEQ ID NO: 387 |
| AAV2 variant | 2709 | WO2018071831 SEQ ID NO: 388 |
| AAV2 variant | 2710 | WO2018071831 SEQ ID NO: 389 |
| AAV2 variant | 2711 | WO2018071831 SEQ ID NO: 390 |
| AAV2 variant | 2712 | WO2018071831 SEQ ID NO: 391 |
| AAV2 variant | 2713 | WO2018071831 SEQ ID NO: 392 |
| AAV2 variant | 2714 | WO2018071831 SEQ ID NO: 393 |
| AAV2 variant | 2715 | WO2018071831 SEQ ID NO: 394 |
| AAV2 variant | 2716 | WO2018071831 SEQ ID NO: 395 |
| AAV2 variant | 2717 | WO2018071831 SEQ ID NO: 396 |
| AAV2 variant | 2718 | WO2018071831 SEQ ID NO: 397 |
| AAV2 variant | 2719 | WO2018071831 SEQ ID NO: 398 |
| AAV2 variant | 2720 | WO2018071831 SEQ ID NO: 399 |
| AAV2 variant | 2721 | WO2018071831 SEQ ID NO: 400 |
| AAV2 variant | 2722 | WO2018071831 SEQ ID NO: 401 |
| AAV2 variant | 2723 | WO2018071831 SEQ ID NO: 402 |
| AAV2 variant | 2724 | WO2018071831 SEQ ID NO: 403 |
| AAV2 variant | 2725 | WO2018071831 SEQ ID NO: 404 |
| AAV2 variant | 2726 | WO2018071831 SEQ ID NO: 405 |
| AAV2 variant | 2727 | WO2018071831 SEQ ID NO: 406 |
| AAV2 variant | 2728 | WO2018071831 SEQ ID NO: 407 |
| AAV2 variant | 2729 | WO2018071831 SEQ ID NO: 408 |
| AAV2 variant | 2730 | WO2018071831 SEQ ID NO: 409 |
| AAV2/3 variant | 2731 | WO2018071831 SEQ ID NO: 435 |
| AAV2/3 variant | 2732 | WO2018071831 SEQ ID NO: 436 |
| AAV2/3 variant | 2733 | WO2018071831 SEQ ID NO: 437 |
| AAV2/3 variant | 2734 | WO2018071831 SEQ ID NO: 438 |
| AAV2/3 variant | 2735 | WO2018071831 SEQ ID NO: 439 |
| AAV2/3 variant | 2736 | WO2018071831 SEQ ID NO: 440 |
| AAV2/3 variant | 2737 | WO2018071831 SEQ ID NO: 441 |
| AAV2/3 variant | 2738 | WO2018071831 SEQ ID NO: 442 |
| AAV2/3 variant | 2739 | WO2018071831 SEQ ID NO: 443 |
| AAV2/3 variant | 2740 | WO2018071831 SEQ ID NO: 444 |
| AAV2/3 variant | 2744 | WO2018071831 SEQ ID NO: 445 |
| AAV2/3 variant | 2742 | WO2018071831 SEQ ID NO: 446 |
| AAV2/3 variant | 2743 | WO2018071831 SEQ ID NO: 447 |
| AAV2/3 variant | 2744 | WO2018071831 SEQ ID NO: 448 |
| AAV2/3 variant | 2745 | WO2018071831 SEQ ID NO: 449 |
| AAV2/3 variant | 2746 | WO2018071831 SEQ ID NO: 450 |
| AAV2/3 variant | 2747 | WO2018071831 SEQ ID NO: 451 |
| AAV2/3 variant | 2748 | WO2018071831 SEQ ID NO: 452 |
| AAV2/3 variant | 2749 | WO2018071831 SEQ ID NO: 453 |
| AAV2/3 variant | 2750 | WO2018071831 SEQ ID NO: 454 |
| AAV2/3 variant | 2751 | WO2018071831 SEQ ID NO: 455 |
| AAV2/3 variant | 2752 | WO2018071831 SEQ ID NO: 456 |
| AAV2/3 variant | 2753 | WO2018071831 SEQ ID NO: 457 |
| AAV2/3 variant | 2754 | WO2018071831 SEQ ID NO: 458 |
| AAV2/3 variant | 2755 | WO2018071831 SEQ ID NO: 459 |
| AAV2/3 variant | 2756 | WO2018071831 SEQ ID NO: 460 |
| AAV2/3 variant | 2757 | WO2018071831 SEQ ID NO: 461 |
| AAV2/3 variant | 2758 | WO2018071831 SEQ ID NO: 462 |
| AAV2/3 variant | 2759 | WO2018071831 SEQ ID NO: 463 |
| AAV2/3 variant | 2760 | WO2018071831 SEQ ID NO: 464 |
| AAV2/3 variant | 2761 | WO2018071831 SEQ ID NO: 465 |
| AAV2/3 variant | 2762 | WO2018071831 SEQ ID NO: 466 |
| AAV2/3 variant | 2763 | WO2018071831 SEQ ID NO: 467 |
| AAV2/3 variant | 2764 | WO2018071831 SEQ ID NO: 468 |
| AAV2/3 variant | 2765 | WO2018071831 SEQ ID NO: 469 |
| AAV2/3 variant | 2766 | WO2018071831 SEQ ID NO: 470 |
| AAV2/3 variant | 2767 | WO2018071831 SEQ ID NO: 471 |
| AAV2/3 variant | 2768 | WO2018071831 SEQ ID NO: 472 |
| AAV2/3 variant | 2769 | WO2018071831 SEQ ID NO: 473 |
| AAV2/3 variant | 2770 | WO2018071831 SEQ ID NO: 474 |
| AAV2/3 variant | 2771 | WO2018071831 SEQ ID NO: 475 |
| AAV2/3 variant | 2772 | WO2018071831 SEQ ID NO: 476 |
| AAV2/3 variant | 2773 | WO2018071831 SEQ ID NO: 477 |
| AAV2/3 variant | 2774 | WO2018071831 SEQ ID NO: 478 |
| AAV2/3 variant | 2775 | WO2018071831 SEQ ID NO: 479 |
| AAV2/3 variant | 2776 | WO2018071831 SEQ ID NO: 480 |
| AAV2/3 variant | 2777 | WO2018071831 SEQ ID NO: 481 |
| AAV2/3 variant | 2778 | WO2018071831 SEQ ID NO: 482 |
| AAV2/3 variant | 2779 | WO2018071831 SEQ ID NO: 483 |
| AAV2/3 variant | 2780 | WO2018071831 SEQ ID NO: 484 |
| AAV2/3 variant | 2781 | WO2018071831 SEQ ID NO: 485 |
| AAV2/3 variant | 2782 | WO2018071831 SEQ ID NO: 486 |
| AAV2/3 variant | 2783 | WO2018071831 SEQ ID NO: 487 |
| AAV2/3 variant | 2784 | WO2018071831 SEQ ID NO: 488 |
| AAV2/3 variant | 2785 | WO2018071831 SEQ ID NO: 489 |
| AAV2/3 variant | 2786 | WO2018071831 SEQ ID NO: 490 |
| AAV2/3 variant | 2787 | WO2018071831 SEQ ID NO: 491 |
| AAV2/3 variant | 2788 | WO2018071831 SEQ ID NO: 492 |
| AAV2/3 variant | 2789 | WO2018071831 SEQ ID NO: 493 |
| AAV2/3 variant | 2790 | WO2018071831 SEQ ID NO: 494 |
| AAV2/3 variant | 2791 | WO2018071831 SEQ ID NO: 495 |
| AAV2/3 variant | 2792 | WO2018071831 SEQ ID NO: 496 |
| AAV2/3 variant | 2793 | WO2018071831 SEQ ID NO: 497 |
| AAV2/3 variant | 2794 | WO2018071831 SEQ ID NO: 498 |
| AAV2/3 variant | 2795 | WO2018071831 SEQ ID NO: 499 |
| AAV2/3 variant | 2796 | WO2018071831 SEQ ID NO: 500 |
| AAV2/3 variant | 2797 | WO2018071831 SEQ ID NO: 501 |
| AAV2/3 variant | 2798 | WO2018071831 SEQ ID NO: 502 |
| AAV2/3 variant | 2799 | WO2018071831 SEQ ID NO: 503 |
| AAV2/3 variant | 2800 | WO2018071831 SEQ ID NO: 504 |
| AAV2/3 variant | 2801 | WO2018071831 SEQ ID NO: 505 |
| AAV2/3 variant | 2802 | WO2018071831 SEQ ID NO: 506 |
| AAV2/3 variant | 2803 | WO2018071831 SEQ ID NO: 507 |
| AAV2/3 variant | 2804 | WO2018071831 SEQ ID NO: 508 |
| AAV2/3 variant | 2805 | WO2018071831 SEQ ID NO: 509 |
| AAV2/3 variant | 2806 | WO2018071831 SEQ ID NO: 510 |
| AAV2/3 variant | 2807 | WO2018071831 SEQ ID NO: 511 |
| AAV2/3 variant | 2808 | WO2018071831 SEQ ID NO: 512 |
| AAV2/3 variant | 2809 | WO2018071831 SEQ ID NO: 513 |
| AAV2/3 variant | 2810 | WO2018071831 SEQ ID NO: 514 |
| AAV2/3 variant | 2811 | WO2018071831 SEQ ID NO: 515 |
| AAV2/3 variant | 2812 | WO2018071831 SEQ ID NO: 516 |
| AAV2/3 variant | 2813 | WO2018071831 SEQ ID NO: 517 |
| AAV2/3 variant | 2814 | WO2018071831 SEQ ID NO: 518 |
| AAV2/3 variant | 2815 | WO2018071831 SEQ ID NO: 519 |
| AAV2/3 variant | 2816 | WO2018071831 SEQ ID NO: 520 |
| AAV2/3 variant | 2817 | WO2018071831 SEQ ID NO: 521 |
| AAV2/3 variant | 2818 | WO2018071831 SEQ ID NO: 522 |
| AAV2/3 variant | 2819 | WO2018071831 SEQ ID NO: 523 |
| AAV2/3 variant | 2820 | WO2018071831 SEQ ID NO: 524 |
| AAV2/3 variant | 2821 | WO2018071831 SEQ ID NO: 525 |
| AAV2/3 variant | 2822 | WO2018071831 SEQ ID NO: 526 |
| AAV2/3 variant | 2823 | WO2018071831 SEQ ID NO: 527 |
| AAV2/3 variant | 2824 | WO2018071831 SEQ ID NO: 528 |
| AAV2/3 variant | 2825 | WO2018071831 SEQ ID NO: 529 |
| AAV2/3 variant | 2826 | WO2018071831 SEQ ID NO: 530 |
| AAV2/3 variant | 2827 | WO2018071831 SEQ ID NO: 531 |
| AAV2/3 variant | 2828 | WO2018071831 SEQ ID NO: 532 |
| AAV2/3 variant | 2829 | WO2018071831 SEQ ID NO: 533 |
| AAV2/3 variant | 2830 | WO2018071831 SEQ ID NO: 534 |
| AAV2/3 variant | 2831 | WO2018071831 SEQ ID NO: 535 |
| AAV2/3 variant | 2832 | WO2018071831 SEQ ID NO: 536 |
| AAV2/3 variant | 2833 | WO2018071831 SEQ ID NO: 537 |
| AAV2/3 variant | 2834 | WO2018071831 SEQ ID NO: 538 |
| AAV2/3 variant | 2835 | WO2018071831 SEQ ID NO: 539 |
| AAV2/3 variant | 2836 | WO2018071831 SEQ ID NO: 540 |
| AAV2/3 variant | 2837 | WO2018071831 SEQ ID NO: 541 |
| AAV2/3 variant | 2838 | WO2018071831 SEQ ID NO: 542 |
| AAV2/3 variant | 2839 | WO2018071831 SEQ ID NO: 543 |
| AAV2/3 variant | 2840 | WO2018071831 SEQ ID NO: 544 |
| AAV2/3 variant | 2841 | WO2018071831 SEQ ID NO: 545 |
| AAV2/3 variant | 2842 | WO2018071831 SEQ ID NO: 546 |
| AAV2/3 variant | 2843 | WO2018071831 SEQ ID NO: 547 |
| AAV2/3 variant | 2844 | WO2018071831 SEQ ID NO: 548 |
| AAV2/3 variant | 2845 | WO2018071831 SEQ ID NO: 549 |
| AAV2/3 variant | 2846 | WO2018071831 SEQ ID NO: 550 |
| AAV2/3 variant | 2847 | WO2018071831 SEQ ID NO: 551 |
| AAV2/3 variant | 2848 | WO2018071831 SEQ ID NO: 552 |
| AAV2/3 variant | 2849 | WO2018071831 SEQ ID NO: 553 |
| AAV2/3 variant | 2850 | WO2018071831 SEQ ID NO: 554 |
| AAV2/3 variant | 2851 | WO2018071831 SEQ ID NO: 555 |
| AAV2/3 variant | 2852 | WO2018071831 SEQ ID NO: 556 |
| AAV2/3 variant | 2853 | WO2018071831 SEQ ID NO: 557 |
| AAV2/3 variant | 2854 | WO2018071831 SEQ ID NO: 558 |
| AAV2/3 variant | 2855 | WO2018071831 SEQ ID NO: 559 |
| AAV2/3 variant | 2856 | WO2018071831 SEQ ID NO: 560 |
| AAV2/3 variant | 2857 | WO2018071831 SEQ ID NO: 561 |
| AAV2/3 variant | 2858 | WO2018071831 SEQ ID NO: 562 |
| AAV2/3 variant | 2859 | WO2018071831 SEQ ID NO: 563 |
| AAV2/3 variant | 2860 | WO2018071831 SEQ ID NO: 564 |
| AAV2/3 variant | 2861 | WO2018071831 SEQ ID NO: 565 |
| AAV2/3 variant | 2862 | WO2018071831 SEQ ID NO: 566 |
| AAV2/3 variant | 2863 | WO2018071831 SEQ ID NO: 567 |
| AAV2/3 variant | 2864 | WO2018071831 SEQ ID NO: 568 |
| AAV2/3 variant | 2865 | WO2018071831 SEQ ID NO: 569 |
| AAV2/3 variant | 2866 | WO2018071831 SEQ ID NO: 570 |
| AAV2/3 variant | 2867 | WO2018071831 SEQ ID NO: 571 |
| AAV2/3 variant | 2868 | WO2018071831 SEQ ID NO: 572 |
| AAV2/3 variant | 2869 | WO2018071831 SEQ ID NO: 573 |
| AAV2/3 variant | 2870 | WO2018071831 SEQ ID NO: 574 |
| AAV2/3 variant | 2871 | WO2018071831 SEQ ID NO: 575 |
| AAV2/3 variant | 2872 | WO2018071831 SEQ ID NO: 576 |
| AAV2/3 variant | 2873 | WO2018071831 SEQ ID NO: 577 |
| AAV2/3 variant | 2874 | WO2018071831 SEQ ID NO: 578 |
| AAV2/3 variant | 2875 | WO2018071831 SEQ ID NO: 579 |
| AAV2/3 variant | 2876 | WO2018071831 SEQ ID NO: 580 |
| AAV2/3 variant | 2877 | WO2018071831 SEQ ID NO: 581 |
| AAV2/3 variant | 2878 | WO2018071831 SEQ ID NO: 582 |
| AAV2/3 variant | 2879 | WO2018071831 SEQ ID NO: 583 |
| AAV2/3 variant | 2880 | WO2018071831 SEQ ID NO: 584 |
| AAV2/3 variant | 2881 | WO2018071831 SEQ ID NO: 585 |
| AAV2/3 variant | 2882 | WO2018071831 SEQ ID NO: 586 |
| AAV2/3 variant | 2883 | WO2018071831 SEQ ID NO: 587 |
| AAV2/3 variant | 2884 | WO2018071831 SEQ ID NO: 588 |
| AAV2/3 variant | 2885 | WO2018071831 SEQ ID NO: 589 |
| AAV2/3 variant | 2886 | WO2018071831 SEQ ID NO: 590 |
| AAV2/3 variant | 2887 | WO2018071831 SEQ ID NO: 591 |
| AAV2/3 variant | 2888 | WO2018071831 SEQ ID NO: 592 |
| AAV2/3 variant | 2889 | WO2018071831 SEQ ID NO: 593 |
| AAV2/3 variant | 2890 | WO2018071831 SEQ ID NO: 594 |
| AAV2/3 variant | 2891 | WO2018071831 SEQ ID NO: 595 |
| AAV2/3 variant | 2892 | WO2018071831 SEQ ID NO: 596 |
| AAV2/3 variant | 2893 | WO2018071831 SEQ ID NO: 597 |
| AAV2/3 variant | 2894 | WO2018071831 SEQ ID NO: 598 |
| AAV2/3 variant | 2895 | WO2018071831 SEQ ID NO: 599 |
| AAV2/3 variant | 2896 | WO2018071831 SEQ ID NO: 600 |
| AAV2/3 variant | 2897 | WO2018071831 SEQ ID NO: 601 |
| AAV2/3 variant | 2898 | WO2018071831 SEQ ID NO: 602 |
| AAV2/3 variant | 2899 | WO2018071831 SEQ ID NO: 603 |
| AAV2/3 variant | 2900 | WO2018071831 SEQ ID NO: 604 |
| AAV2/3 variant | 2901 | WO2018071831 SEQ ID NO: 605 |
| AAV2/3 variant | 2902 | WO2018071831 SEQ ID NO: 606 |
| AAV2/3 variant | 2903 | WO2018071831 SEQ ID NO: 607 |
| AAV2/3 variant | 2904 | WO2018071831 SEQ ID NO: 608 |
| AAV2/3 variant | 2905 | WO2018071831 SEQ ID NO: 609 |
| AAV2/3 variant | 2906 | WO2018071831 SEQ ID NO: 610 |
| AAV2/3 variant | 2907 | WO2018071831 SEQ ID NO: 611 |
| AAV2/3 variant | 2908 | WO2018071831 SEQ ID NO: 612 |
| AAV2/3 variant | 2909 | WO2018071831 SEQ ID NO: 613 |
| AAV2/3 variant | 2910 | WO2018071831 SEQ ID NO: 614 |
| AAV2/3 variant | 2911 | WO2018071831 SEQ ID NO: 615 |
| AAV2/3 variant | 2912 | WO2018071831 SEQ ID NO: 616 |
| AAV2/3 variant | 2913 | WO2018071831 SEQ ID NO: 617 |
| AAV2/3 variant | 2914 | WO2018071831 SEQ ID NO: 618 |
| AAV2/3 variant | 2915 | WO2018071831 SEQ ID NO: 619 |
| AAV2/3 variant | 2916 | WO2018071831 SEQ ID NO: 620 |
| AAV2/3 variant | 2917 | WO2018071831 SEQ ID NO: 621 |
| AAV2/3 variant | 2918 | WO2018071831 SEQ ID NO: 622 |
| AAV2/3 variant | 2919 | WO2018071831 SEQ ID NO: 623 |
| AAV2/3 variant | 2920 | WO2018071831 SEQ ID NO: 624 |
| AAV2/3 variant | 2921 | WO2018071831 SEQ ID NO: 625 |
| AAV2/3 variant | 2922 | WO2018071831 SEQ ID NO: 626 |
| AAV2/3 variant | 2923 | WO2018071831 SEQ ID NO: 627 |
| AAV2/3 variant | 2924 | WO2018071831 SEQ ID NO: 628 |
| AAV8 Variant | 2925 | WO2018071831 SEQ ID NO: 629 |
| AAV8 Variant | 2926 | WO2018071831 SEQ ID NO: 630 |
| AAV8 Variant | 2927 | WO2018071831 SEQ ID NO: 631 |
| AAV8 Variant | 2928 | WO2018071831 SEQ ID NO: 632 |
| AAV8 Variant | 2929 | WO2018071831 SEQ ID NO: 633 |
| AAV8 Variant | 2930 | WO2018071831 SEQ ID NO: 634 |
| AAV8 Variant | 2931 | WO2018071831 SEQ ID NO: 635 |
| AAV8 Variant | 2932 | WO2018071831 SEQ ID NO: 636 |
| AAV8 Variant | 2933 | WO2018071831 SEQ ID NO: 637 |
| AAV8 Variant | 2934 | WO2018071831 SEQ ID NO: 638 |
| AAV8 Variant | 2935 | WO2018071831 SEQ ID NO: 639 |
| AAV8 Variant | 2936 | WO2018071831 SEQ ID NO: 640 |
| AAV8 Variant | 2937 | WO2018071831 SEQ ID NO: 641 |
| AAV8 Variant | 2938 | WO2018071831 SEQ ID NO: 642 |
| AAV8 Variant | 2939 | WO2018071831 SEQ ID NO: 643 |
| AAV8 Variant | 2940 | WO2018071831 SEQ ID NO: 644 |
| AAV8 Variant | 2941 | WO2018071831 SEQ ID NO: 645 |
| AAV8 Variant | 2942 | WO2018071831 SEQ ID NO: 646 |
| AAV8 Variant | 2943 | WO2018071831 SEQ ID NO: 647 |
| AAV8 Variant | 2944 | WO2018071831 SEQ ID NO: 648 |
| AAV8 Variant | 2945 | WO2018071831 SEQ ID NO: 649 |
| AAV8 Variant | 2946 | WO2018071831 SEQ ID NO: 650 |
| AAV8 Variant | 2947 | WO2018071831 SEQ ID NO: 651 |
| AAV8 Variant | 2948 | WO2018071831 SEQ ID NO: 652 |
| AAV8 Variant | 2949 | WO2018071831 SEQ ID NO: 653 |
| AAV8 Variant | 2950 | WO2018071831 SEQ ID NO: 654 |
| AAV8 Variant | 2951 | WO2018071831 SEQ ID NO: 655 |
| AAV8 Variant | 2952 | WO2018071831 SEQ ID NO: 656 |
| AAV8 Variant | 2953 | WO2018071831 SEQ ID NO: 657 |
| AAV8 Variant | 2954 | WO2018071831 SEQ ID NO: 658 |
| AAV8 Variant | 2955 | WO2018071831 SEQ ID NO: 659 |
| AAV8 Variant | 2956 | WO2018071831 SEQ ID NO: 660 |
| AAV8 Variant | 2957 | WO2018071831 SEQ ID NO: 661 |
| AAV8 Variant | 2958 | WO2018071831 SEQ ID NO: 662 |
| AAV8 Variant | 2959 | WO2018071831 SEQ ID NO: 663 |
| AAV8 Variant | 2960 | WO2018071831 SEQ ID NO: 664 |
| AAV8 Variant | 2961 | WO2018071831 SEQ ID NO: 665 |
| AAV8 Variant | 2962 | WO2018071831 SEQ ID NO: 666 |
| AAV8 Variant | 2963 | WO2018071831 SEQ ID NO: 667 |
| AAV8 Variant | 2964 | WO2018071831 SEQ ID NO: 668 |
| AAV8 Variant | 2965 | WO2018071831 SEQ ID NO: 669 |
| AAV8 Variant | 2966 | WO2018071831 SEQ ID NO: 670 |
| AAV8 Variant | 2967 | WO2018071831 SEQ ID NO: 671 |
| AAV8 Variant | 2968 | WO2018071831 SEQ ID NO: 672 |
| AAV8 Variant | 2969 | WO2018071831 SEQ ID NO: 673 |
| AAV8 Variant | 2970 | WO2018071831 SEQ ID NO: 674 |
| AAV8 Variant | 2971 | WO2018071831 SEQ ID NO: 675 |
| AAV8 Variant | 2972 | WO2018071831 SEQ ID NO: 676 |
| AAV8 Variant | 2973 | WO2018071831 SEQ ID NO: 677 |
| AAV8 Variant | 2974 | WO2018071831 SEQ ID NO: 678 |
| AAV8 Variant | 2975 | WO2018071831 SEQ ID NO: 679 |
| AAV8 Variant | 2976 | WO2018071831 SEQ ID NO: 680 |
| AAV8 Variant | 2977 | WO2018071831 SEQ ID NO: 681 |
| AAV8 Variant | 2978 | WO2018071831 SEQ ID NO: 682 |
| AAV8 Variant | 2979 | WO2018071831 SEQ ID NO: 683 |
| AAV8 Variant | 2980 | WO2018071831 SEQ ID NO: 684 |
| AAV8 Variant | 2981 | WO2018071831 SEQ ID NO: 685 |
| AAV8 Variant | 2982 | WO2018071831 SEQ ID NO: 686 |
| AAV8 Variant | 2983 | WO2018071831 SEQ ID NO: 687 |
| AAV8 Variant | 2984 | WO2018071831 SEQ ID NO: 688 |
| AAV8 Variant | 2985 | WO2018071831 SEQ ID NO: 689 |
| AAV8 Variant | 2986 | WO2018071831 SEQ ID NO: 690 |
| AAV8 Variant | 2987 | WO2018071831 SEQ ID NO: 691 |
| AAV8 Variant | 2988 | WO2018071831 SEQ ID NO: 692 |
| AAV8 Variant | 2989 | WO2018071831 SEQ ID NO: 693 |
| AAV8 Variant | 2990 | WO2018071831 SEQ ID NO: 694 |
| AAV8 Variant | 2991 | WO2018071831 SEQ ID NO: 695 |
| AAV8 Variant | 2992 | WO2018071831 SEQ ID NO: 696 |
| AAV8 Variant | 2993 | WO2018071831 SEQ ID NO: 697 |
| AAV8 Variant | 2994 | WO2018071831 SEQ ID NO: 698 |
| AAV8 Variant | 2995 | WO2018071831 SEQ ID NO: 699 |
| AAV8 Variant | 2996 | WO2018071831 SEQ ID NO: 700 |
| AAV8 Variant | 2997 | WO2018071831 SEQ ID NO: 701 |
| AAV8 Variant | 2998 | WO2018071831 SEQ ID NO: 702 |
| AAV8 Variant | 2999 | WO2018071831 SEQ ID NO: 703 |
| AAV8 Variant | 3000 | WO2018071831 SEQ ID NO: 704 |
| AAV8 Variant | 3001 | WO2018071831 SEQ ID NO: 705 |
| AAV8 Variant | 3002 | WO2018071831 SEQ ID NO: 706 |
| AAV8 Variant | 3003 | WO2018071831 SEQ ID NO: 707 |
| AAV8 Variant | 3004 | WO2018071831 SEQ ID NO: 708 |
| AAV8 Variant | 3005 | WO2018071831 SEQ ID NO: 709 |
| AAV8 Variant | 3006 | WO2018071831 SEQ ID NO: 710 |
| AAV8 Variant | 3007 | WO2018071831 SEQ ID NO: 711 |
| AAV8 Variant | 3008 | WO2018071831 SEQ ID NO: 712 |
| AAV8 Variant | 3009 | WO2018071831 SEQ ID NO: 713 |
| AAV8 Variant | 3010 | WO2018071831 SEQ ID NO: 714 |
| AAV8 Variant | 3011 | WO2018071831 SEQ ID NO: 715 |
| AAV8 Variant | 3012 | WO2018071831 SEQ ID NO: 716 |
| AAV8 Variant | 3013 | WO2018071831 SEQ ID NO: 717 |
| AAV8 Variant | 3014 | WO2018071831 SEQ ID NO: 718 |
| AAV8 Variant | 3015 | WO2018071831 SEQ ID NO: 719 |
| AAV8 Variant | 3016 | WO2018071831 SEQ ID NO: 720 |
| AAV8 Variant | 3017 | WO2018071831 SEQ ID NO: 721 |
| AAV8 Variant | 3018 | WO2018071831 SEQ ID NO: 722 |
| AAV8 Variant | 3019 | WO2018071831 SEQ ID NO: 723 |
| AAV8 Variant | 3020 | WO2018071831 SEQ ID NO: 724 |
| AAV8 Variant | 3021 | WO2018071831 SEQ ID NO: 725 |
| AAV8 Variant | 3022 | WO2018071831 SEQ ID NO: 726 |
| AAV8 Variant | 3023 | WO2018071831 SEQ ID NO: 727 |
| AAV8 Variant | 3024 | WO2018071831 SEQ ID NO: 728 |
| AAV8 Variant | 3025 | WO2018071831 SEQ ID NO: 729 |
| AAV8 Variant | 3026 | WO2018071831 SEQ ID NO: 730 |
| AAV8 Variant | 3027 | WO2018071831 SEQ ID NO: 731 |
| AAV8 Variant | 3028 | WO2018071831 SEQ ID NO: 732 |
| AAV8 Variant | 3029 | WO2018071831 SEQ ID NO: 733 |
| AAV8 Variant | 3030 | WO2018071831 SEQ ID NO: 734 |
| AAV8 Variant | 3031 | WO2018071831 SEQ ID NO: 735 |
| AAV8 Variant | 3032 | WO2018071831 SEQ ID NO: 736 |
| AAV8 Variant | 3033 | WO2018071831 SEQ ID NO: 737 |
| AAV8 Variant | 3034 | WO2018071831 SEQ ID NO: 738 |
| AAV8 Variant | 3035 | WO2018071831 SEQ ID NO: 739 |
| AAV8 Variant | 3036 | WO2018071831 SEQ ID NO: 740 |
| AAV8 Variant | 3037 | WO2018071831 SEQ ID NO: 741 |
| AAV8 Variant | 3038 | WO2018071831 SEQ ID NO: 742 |
| AAV8 Variant | 3039 | WO2018071831 SEQ ID NO: 743 |
| AAV8 Variant | 3040 | WO2018071831 SEQ ID NO: 744 |
| AAV8 Variant | 3041 | WO2018071831 SEQ ID NO: 745 |
| AAV8 Variant | 3042 | WO2018071831 SEQ ID NO: 746 |
| AAV8 Variant | 3043 | WO2018071831 SEQ ID NO: 747 |
| AAV8 Variant | 3044 | WO2018071831 SEQ ID NO: 748 |
| AAV8 Variant | 3045 | WO2018071831 SEQ ID NO: 749 |
| AAV8 Variant | 3046 | WO2018071831 SEQ ID NO: 750 |
| AAV8 Variant | 3047 | WO2018071831 SEQ ID NO: 751 |
| AAV8 Variant | 3048 | WO2018071831 SEQ ID NO: 752 |
| AAV8 Variant | 3049 | WO2018071831 SEQ ID NO: 753 |
| AAV8 Variant | 3050 | WO2018071831 SEQ ID NO: 754 |
| AAV8 Variant | 3051 | WO2018071831 SEQ ID NO: 755 |
| AAV8 Variant | 3052 | WO2018071831 SEQ ID NO: 756 |
| AAV8 Variant | 3053 | WO2018071831 SEQ ID NO: 757 |
| AAV8 Variant | 3054 | WO2018071831 SEQ ID NO: 758 |
| AAV8 Variant | 3055 | WO2018071831 SEQ ID NO: 759 |
| AAV8 Variant | 3056 | WO2018071831 SEQ ID NO: 760 |
| AAV8 Variant | 3057 | WO2018071831 SEQ ID NO: 761 |
| AAV8 Variant | 3058 | WO2018071831 SEQ ID NO: 762 |
| AAV8 Variant | 3059 | WO2018071831 SEQ ID NO: 763 |
| AAV8 Variant | 3060 | WO2018071831 SEQ ID NO: 764 |
| AAV8 Variant | 3061 | WO2018071831 SEQ ID NO: 765 |
| AAV8 Variant | 3062 | WO2018071831 SEQ ID NO: 766 |
| AAV8 Variant | 3063 | WO2018071831 SEQ ID NO: 767 |
| AAV8 Variant | 3064 | WO2018071831 SEQ ID NO: 768 |
| AAV8 Variant | 3065 | WO2018071831 SEQ ID NO: 769 |
| AAV8 Variant | 3066 | WO2018071831 SEQ ID NO: 770 |
| AAV8 Variant | 3067 | WO2018071831 SEQ ID NO: 771 |
| AAV8 Variant | 3068 | WO2018071831 SEQ ID NO: 772 |
| AAV8 Variant | 3069 | WO2018071831 SEQ ID NO: 773 |
| AAV8 Variant | 3070 | WO2018071831 SEQ ID NO: 774 |
| AAV8 Variant | 3071 | WO2018071831 SEQ ID NO: 775 |
| AAV8 Variant | 3072 | WO2018071831 SEQ ID NO: 776 |
| AAV8 Variant | 3073 | WO2018071831 SEQ ID NO: 777 |
| AAV8 Variant | 3074 | WO2018071831 SEQ ID NO: 778 |
| AAV8 Variant | 3075 | WO2018071831 SEQ ID NO: 779 |
| AAV8 Variant | 3076 | WO2018071831 SEQ ID NO: 780 |
| AAV8 Variant | 3077 | WO2018071831 SEQ ID NO: 781 |
| AAV8 Variant | 3078 | WO2018071831 SEQ ID NO: 782 |
| AAV8 Variant | 3079 | WO2018071831 SEQ ID NO: 783 |
| AAV8 Variant | 3080 | WO2018071831 SEQ ID NO: 784 |
| AAV8 Variant | 3081 | WO2018071831 SEQ ID NO: 785 |
| AAV8 Variant | 3082 | WO2018071831 SEQ ID NO: 786 |
| AAV8 Variant | 3083 | WO2018071831 SEQ ID NO: 787 |
| AAV8 Variant | 3084 | WO2018071831 SEQ ID NO: 788 |
| AAV8 Variant | 3085 | WO2018071831 SEQ ID NO: 789 |
| AAV8 Variant | 3086 | WO2018071831 SEQ ID NO: 790 |
| AAV8 Variant | 3087 | WO2018071831 SEQ ID NO: 791 |
| AAV8 Variant | 3088 | WO2018071831 SEQ ID NO: 792 |
| AAV8 Variant | 3089 | WO2018071831 SEQ ID NO: 793 |
| AAV8 Variant | 3090 | WO2018071831 SEQ ID NO: 794 |
| AAV8 Variant | 3091 | WO2018071831 SEQ ID NO: 795 |
| AAV8 Variant | 3092 | WO2018071831 SEQ ID NO: 796 |
| AAV8 Variant | 3093 | WO2018071831 SEQ ID NO: 797 |
| AAV8 Variant | 3094 | WO2018071831 SEQ ID NO: 798 |
| AAV8 Variant | 3095 | WO2018071831 SEQ ID NO: 799 |
| AAV8 Variant | 3096 | WO2018071831 SEQ ID NO: 800 |
| AAV8 Variant | 3097 | WO2018071831 SEQ ID NO: 801 |
| AAV8 Variant | 3098 | WO2018071831 SEQ ID NO: 802 |
| AAV8 Variant | 3099 | WO2018071831 SEQ ID NO: 803 |
| AAV8 Variant | 3100 | WO2018071831 SEQ ID NO: 804 |
| AAV8 Variant | 3101 | WO2018071831 SEQ ID NO: 805 |
| AAV8 Variant | 3102 | WO2018071831 SEQ ID NO: 806 |
| AAV8 Variant | 3103 | WO2018071831 SEQ ID NO: 807 |
| AAV8 Variant | 3104 | WO2018071831 SEQ ID NO: 808 |
| AAV8 Variant | 3105 | WO2018071831 SEQ ID NO: 809 |
| AAV8 Variant | 3106 | WO2018071831 SEQ ID NO: 810 |
| AAV8 Variant | 3107 | WO2018071831 SEQ ID NO: 811 |
| AAV8 Variant | 3108 | WO2018071831 SEQ ID NO: 812 |
| AAV8 Variant | 3109 | WO2018071831 SEQ ID NO: 813 |
| AAV8 Variant | 3110 | WO2018071831 SEQ ID NO: 814 |
| AAV8 Variant | 3111 | WO2018071831 SEQ ID NO: 815 |
| AAV8 Variant | 3112 | WO2018071831 SEQ ID NO: 816 |
| AAV8 Variant | 3113 | WO2018071831 SEQ ID NO: 817 |
| AAV8 Variant | 3114 | WO2018071831 SEQ ID NO: 818 |
| AAV8 Variant | 3115 | WO2018071831 SEQ ID NO: 819 |
| AAV8 Variant | 3116 | WO2018071831 SEQ ID NO: 820 |
| AAV8 Variant | 3117 | WO2018071831 SEQ ID NO: 821 |
| AAV8 Variant | 3118 | WO2018071831 SEQ ID NO: 822 |
| AAV8 Variant | 3119 | WO2018071831 SEQ ID NO: 823 |
| AAV8 Variant | 3120 | WO2018071831 SEQ ID NO: 824 |
| AAV8 Variant | 3121 | WO2018071831 SEQ ID NO: 825 |
| AAV8 Variant | 3122 | WO2018071831 SEQ ID NO: 826 |
| AAV8 Variant | 3123 | WO2018071831 SEQ ID NO: 827 |
| AAV8 Variant | 3124 | WO2018071831 SEQ ID NO: 828 |
| AAV8 Variant | 3125 | WO2018071831 SEQ ID NO: 829 |
| AAV8 Variant | 3126 | WO2018071831 SEQ ID NO: 830 |
| AAV8 Variant | 3127 | WO2018071831 SEQ ID NO: 831 |
| AAV8 Variant | 3128 | WO2018071831 SEQ ID NO: 832 |
| AAV8 Variant | 3129 | WO2018071831 SEQ ID NO: 833 |
| AAV8 Variant | 3130 | WO2018071831 SEQ ID NO: 834 |
| AAV8 Variant | 3131 | WO2018071831 SEQ ID NO: 835 |
| AAV8 Variant | 3132 | WO2018071831 SEQ ID NO: 836 |
| AAV2 variant | 3133 | WO2018071831 SEQ ID NO: 837 |
| AAV2 variant | 3134 | WO2018071831 SEQ ID NO: 838 |
| AAV2 variant | 3135 | WO2018071831 SEQ ID NO: 839 |
| AAV2 variant | 3136 | WO2018071831 SEQ ID NO: 840 |
| AAV2 variant | 3137 | WO2018071831 SEQ ID NO: 841 |
| AAV2 variant | 3138 | WO2018071831 SEQ ID NO: 842 |
| AAV2 variant | 3139 | WO2018071831 SEQ ID NO: 843 |
| AAV2 variant | 3140 | WO2018071831 SEQ ID NO: 844 |
| AAV2 variant | 3141 | WO2018071831 SEQ ID NO: 845 |
| AAV2 variant | 3142 | WO2018071831 SEQ ID NO: 846 |
| AAV2 variant | 3143 | WO2018071831 SEQ ID NO: 847 |
| AAV2 variant | 3144 | WO2018071831 SEQ ID NO: 848 |
| AAV2 variant | 3145 | WO2018071831 SEQ ID NO: 849 |
| AAV2 variant | 3146 | WO2018071831 SEQ ID NO: 850 |
| AAV2 variant | 3147 | WO2018071831 SEQ ID NO: 851 |
| AAV2 variant | 3148 | WO2018071831 SEQ ID NO: 852 |
| AAV8 B1 | 3149 | WO2018071831 SEQ ID NO: 853 |
| AAV8 B2 | 3150 | WO2018071831 SEQ ID NO: 854 |
| AAV8 B3 | 3151 | WO2018071831 SEQ ID NO: 855 |
| AAV8 B4 | 3152 | WO2018071831 SEQ ID NO: 856 |
| AAV8 B12 | 3153 | WO2018071831 SEQ ID NO: 857 |
| AAV8 B18 | 3154 | WO2018071831 SEQ ID NO: 858 |
| AAV8 B24 | 3155 | WO2018071831 SEQ ID NO: 859 |
| AAV8 B41 | 3156 | WO2018071831 SEQ ID NO: 860 |
| AAV8 B44 | 3157 | WO2018071831 SEQ ID NO: 861 |
| AAV8 B45 | 3158 | WO2018071831 SEQ ID NO: 862 |
| AAV8 B46 | 3159 | WO2018071831 SEQ ID NO: 863 |
| AAV8 B60 | 3160 | WO2018071831 SEQ ID NO: 864 |
| AAV8 B61 | 3161 | WO2018071831 SEQ ID NO: 865 |
| AAV8 B62 | 3162 | WO2018071831 SEQ ID NO: 866 |
| AAV8 B63 | 3163 | WO2018071831 SEQ ID NO: 867 |
| AAV8 B64 | 3164 | WO2018071831 SEQ ID NO: 868 |
| AAV variant | 3165 | WO2018071831 SEQ ID NO: 876 |
| AAV variant | 3166 | WO2018071831 SEQ ID NO: 877 |
| AAV variant | 3167 | WO2018071831 SEQ ID NO: 878 |
| AAV variant | 3168 | WO2018071831 SEQ ID NO: 879 |
| AAV variant | 3169 | WO2018071831 SEQ ID NO: 880 |
| AAV variant | 3170 | WO2018071831 SEQ ID NO: 881 |
| AAV variant | 3171 | WO2018071831 SEQ ID NO: 882 |
| AAV variant | 3172 | WO2018071831 SEQ ID NO: 883 |
| AAV variant | 3173 | WO2018071831 SEQ ID NO: 884 |
| AAV variant | 3174 | WO2018071831 SEQ ID NO: 885 |
| AAV variant | 3175 | WO2018071831 SEQ ID NO: 886 |
| AAV variant | 3176 | WO2018071831 SEQ ID NO: 887 |
| AAV variant | 3177 | WO2018071831 SEQ ID NO: 888 |
| AAV variant | 3178 | WO2018071831 SEQ ID NO: 889 |
| AAV variant | 3179 | WO2018071831 SEQ ID NO: 890 |
| AAV variant | 3180 | WO2018071831 SEQ ID NO: 891 |
| AAV variant | 3181 | WO2018071831 SEQ ID NO: 892 |
| AAV variant | 3182 | WO2018071831 SEQ ID NO: 893 |
| AAV variant | 3183 | WO2018071831 SEQ ID NO: 894 |
| AAV variant | 3184 | WO2018071831 SEQ ID NO: 895 |
| AAV variant | 3185 | WO2018071831 SEQ ID NO: 896 |
| AAV variant | 3186 | WO2018071831 SEQ ID NO: 897 |
| AAV variant | 3187 | WO2018071831 SEQ ID NO: 898 |
| AAV variant | 3188 | WO2018071831 SEQ ID NO: 899 |
| AAV variant | 3189 | WO2018071831 SEQ ID NO: 900 |
| AAV variant | 3190 | WO2018071831 SEQ ID NO: 901 |
| AAV variant | 3191 | WO2018071831 SEQ ID NO: 902 |
| AAV variant | 3192 | WO2018071831 SEQ ID NO: 903 |
| AAV variant | 3193 | WO2018071831 SEQ ID NO: 904 |
| AAV variant | 3194 | WO2018071831 SEQ ID NO: 905 |
| AAV variant | 3195 | WO2018071831 SEQ ID NO: 906 |
| AAV variant | 3196 | WO2018071831 SEQ ID NO: 907 |
| AAV variant | 3197 | WO2018071831 SEQ ID NO: 908 |
| AAV variant | 3198 | WO2018071831 SEQ ID NO: 909 |
| AAV variant | 3199 | WO2018071831 SEQ ID NO: 910 |
| AAV variant | 3200 | WO2018071831 SEQ ID NO: 911 |
| AAV variant | 3201 | WO2018071831 SEQ ID NO: 912 |
| AAV variant | 3202 | WO2018071831 SEQ ID NO: 913 |
| AAV variant | 3203 | WO2018071831 SEQ ID NO: 914 |
| AAV variant | 3204 | WO2018071831 SEQ ID NO: 915 |
| AAV variant | 3205 | WO2018071831 SEQ ID NO: 916 |
| AAV variant | 3206 | WO2018071831 SEQ ID NO: 917 |
| AAV variant | 3207 | WO2018071831 SEQ ID NO: 918 |
| AAV variant | 3208 | WO2018071831 SEQ ID NO: 919 |
| AAV variant | 3209 | WO2018071831 SEQ ID NO: 920 |
| AAV variant | 3210 | WO2018071831 SEQ ID NO: 921 |
| AAV variant | 3211 | WO2018071831 SEQ ID NO: 922 |
| AAV variant | 3212 | WO2018071831 SEQ ID NO: 923 |
| AAV variant | 3213 | WO2018071831 SEQ ID NO: 924 |
| AAV variant | 3214 | WO2018071831 SEQ ID NO: 925 |
| AAV variant | 3215 | WO2018071831 SEQ ID NO: 926 |
| AAV variant | 3216 | WO2018071831 SEQ ID NO: 927 |
| AAV variant | 3217 | WO2018071831 SEQ ID NO: 928 |
| AAV variant | 3218 | WO2018071831 SEQ ID NO: 929 |
| AAV variant | 3219 | WO2018071831 SEQ ID NO: 930 |
| AAV variant | 3220 | WO2018071831 SEQ ID NO: 931 |
| AAV variant | 3221 | WO2018071831 SEQ ID NO: 932 |
| AAV variant | 3222 | WO2018071831 SEQ ID NO: 933 |
| AAV variant | 3223 | WO2018071831 SEQ ID NO: 934 |
| AAV variant | 3224 | WO2018071831 SEQ ID NO: 935 |
| AAV variant | 3225 | WO2018071831 SEQ ID NO: 936 |
| AAV variant | 3226 | WO2018071831 SEQ ID NO: 937 |
| AAV variant | 3227 | WO2018071831 SEQ ID NO: 938 |
| AAV variant | 3228 | WO2018071831 SEQ ID NO: 939 |
| AAV variant | 3229 | WO2018071831 SEQ ID NO: 940 |
| AAV variant | 3230 | WO2018071831 SEQ ID NO: 941 |
| AAV variant | 3231 | WO2018071831 SEQ ID NO: 942 |
| AAV variant | 3232 | WO2018071831 SEQ ID NO: 943 |
| AAV variant | 3233 | WO2018071831 SEQ ID NO: 944 |
| AAV variant | 3234 | WO2018071831 SEQ ID NO: 945 |
| AAV variant | 3235 | WO2018071831 SEQ ID NO: 946 |
| AAV variant | 3236 | WO2018071831 SEQ ID NO: 947 |
| AAV variant | 3237 | WO2018071831 SEQ ID NO: 948 |
| AAV variant | 3238 | WO2018071831 SEQ ID NO: 949 |
| AAV variant | 3239 | WO2018071831 SEQ ID NO: 950 |
| AAV variant | 3240 | WO2018071831 SEQ ID NO: 951 |
| AAV variant | 3241 | WO2018071831 SEQ ID NO: 952 |
| AAV variant | 3242 | WO2018071831 SEQ ID NO: 953 |
| AAV variant | 3243 | WO2018071831 SEQ ID NO: 954 |
| AAV variant | 3244 | WO2018071831 SEQ ID NO: 955 |
| AAV variant | 3245 | WO2018071831 SEQ ID NO: 956 |
| AAV variant | 3246 | WO2018071831 SEQ ID NO: 957 |
| AAV variant | 3247 | WO2018071831 SEQ ID NO: 958 |
| AAV variant | 3248 | WO2018071831 SEQ ID NO: 959 |
| AAV variant | 3249 | WO2018071831 SEQ ID NO: 960 |
| AAV variant | 3250 | WO2018071831 SEQ ID NO: 961 |
| AAV variant | 3251 | WO2018071831 SEQ ID NO: 962 |
| AAV variant | 3252 | WO2018071831 SEQ ID NO: 963 |
| AAV variant | 3253 | WO2018071831 SEQ ID NO: 964 |
| AAV variant | 3254 | WO2018071831 SEQ ID NO: 965 |
| AAV variant | 3255 | WO2018071831 SEQ ID NO: 966 |
| AAV variant | 3256 | WO2018071831 SEQ ID NO: 967 |
| AAV variant | 3257 | WO2018071831 SEQ ID NO: 968 |
| AAV variant | 3258 | WO2018071831 SEQ ID NO: 969 |
| AAV variant | 3259 | WO2018071831 SEQ ID NO: 970 |
| AAV variant | 3260 | WO2018071831 SEQ ID NO: 971 |
| AAV variant | 3261 | WO2018071831 SEQ ID NO: 972 |
| AAV variant | 3262 | WO2018071831 SEQ ID NO: 973 |
| AAV variant | 3263 | WO2018071831 SEQ ID NO: 974 |
| AAV variant | 3264 | WO2018071831 SEQ ID NO: 975 |
| AAV variant | 3265 | WO2018071831 SEQ ID NO: 976 |
| AAV variant | 3266 | WO2018071831 SEQ ID NO: 977 |
| AAV variant | 3267 | WO2018071831 SEQ ID NO: 978 |
| AAV variant | 3268 | WO2018071831 SEQ ID NO: 979 |
| AAV variant | 3269 | WO2018071831 SEQ ID NO: 980 |
| AAV variant | 3270 | WO2018071831 SEQ ID NO: 981 |
| AAV variant | 3271 | WO2018071831 SEQ ID NO: 982 |
| AAV variant | 3272 | WO2018071831 SEQ ID NO: 983 |
| AAV variant | 3273 | WO2018071831 SEQ ID NO: 984 |
| AAV variant | 3274 | WO2018071831 SEQ ID NO: 985 |
| AAV variant | 3275 | WO2018071831 SEQ ID NO: 986 |
| AAV variant | 3276 | WO2018071831 SEQ ID NO: 987 |
| AAV variant | 3277 | WO2018071831 SEQ ID NO: 988 |
| AAV variant | 3278 | WO2018071831 SEQ ID NO: 989 |
| AAV variant | 3279 | WO2018071831 SEQ ID NO: 990 |
| AAV variant | 3280 | WO2018071831 SEQ ID NO: 991 |
| AAV variant | 3281 | WO2018071831 SEQ ID NO: 992 |
| AAV variant | 3282 | WO2018071831 SEQ ID NO: 993 |
| AAV variant | 3283 | WO2018071831 SEQ ID NO: 994 |
| AAV variant | 3284 | WO2018071831 SEQ ID NO: 995 |
| AAV variant | 3285 | WO2018071831 SEQ ID NO: 996 |
| AAV variant | 3286 | WO2018071831 SEQ ID NO: 997 |
| AAV variant | 3287 | WO2018071831 SEQ ID NO: 998 |
| AAV variant | 3288 | WO2018071831 SEQ ID NO: 999 |
| AAV variant | 3289 | WO2018071831 SEQ ID NO: 1000 |
| AAV variant | 3290 | WO2018071831 SEQ ID NO: 1001 |
| AAV variant | 3291 | WO2018071831 SEQ ID NO: 1002 |
| AAV variant | 3292 | WO2018071831 SEQ ID NO: 1003 |
| AAV variant | 3293 | WO2018071831 SEQ ID NO: 1004 |
| AAV variant | 3294 | WO2018071831 SEQ ID NO: 1005 |
| AAV variant | 3295 | WO2018071831 SEQ ID NO: 1006 |
| AAV variant | 3296 | WO2018071831 SEQ ID NO: 1007 |
| AAV variant | 3297 | WO2018071831 SEQ ID NO: 1008 |
| AAV variant | 3298 | WO2018071831 SEQ ID NO: 1009 |
| AAV variant | 3299 | WO2018071831 SEQ ID NO: 1010 |
| AAV variant | 3300 | WO2018071831 SEQ ID NO: 1011 |
| AAV variant | 3301 | WO2018071831 SEQ ID NO: 1012 |
| AAV variant | 3302 | WO2018071831 SEQ ID NO: 1013 |
| AAV variant | 3303 | WO2018071831 SEQ ID NO: 1014 |
| AAV variant | 3304 | WO2018071831 SEQ ID NO: 1015 |
| AAV variant | 3305 | WO2018071831 SEQ ID NO: 1016 |
| AAV variant | 3306 | WO2018071831 SEQ ID NO: 1017 |
| AAV variant | 3307 | WO2018071831 SEQ ID NO: 1018 |
| AAV variant | 3308 | WO2018071831 SEQ ID NO: 1019 |
| AAV variant | 3309 | WO2018071831 SEQ ID NO: 1020 |
| AAV variant | 3310 | WO2018071831 SEQ ID NO: 1021 |
| AAV variant | 3311 | WO2018071831 SEQ ID NO: 1022 |
| AAV variant | 3312 | WO2018071831 SEQ ID NO: 1023 |
| AAV variant | 3313 | WO2018071831 SEQ ID NO: 1024 |
| AAV variant | 3314 | WO2018071831 SEQ ID NO: 1025 |
| AAV variant | 3315 | WO2018071831 SEQ ID NO: 1026 |
| AAV variant | 3316 | WO2018071831 SEQ ID NO: 1027 |
| AAV variant | 3317 | WO2018071831 SEQ ID NO: 1028 |
| AAV variant | 3318 | WO2018071831 SEQ ID NO: 1029 |
| AAV variant | 3319 | WO2018071831 SEQ ID NO: 1030 |
| AAV variant | 3320 | WO2018071831 SEQ ID NO: 1031 |
| AAV variant | 3321 | WO2018071831 SEQ ID NO: 1032 |
| AAV variant | 3322 | WO2018071831 SEQ ID NO: 1033 |
| AAV variant | 3323 | WO2018071831 SEQ ID NO: 1034 |
| AAV variant | 3324 | WO2018071831 SEQ ID NO: 1035 |
| AAV variant | 3325 | WO2018071831 SEQ ID NO: 1036 |
| AAV variant | 3326 | WO2018071831 SEQ ID NO: 1037 |
| AAV variant | 3327 | WO2018071831 SEQ ID NO: 1038 |
| AAV variant | 3328 | WO2018071831 SEQ ID NO: 1039 |
| AAV variant | 3329 | WO2018071831 SEQ ID NO: 1040 |
| AAV variant | 3330 | WO2018071831 SEQ ID NO: 1041 |
| AAV variant | 3331 | WO2018071831 SEQ ID NO: 1042 |
| AAV variant | 3332 | WO2018071831 SEQ ID NO: 1043 |
| AAV variant | 3333 | WO2018071831 SEQ ID NO: 1044 |
| AAV variant | 3334 | WO2018071831 SEQ ID NO: 1045 |
| AAV variant | 3335 | WO2018071831 SEQ ID NO: 1046 |
| AAV variant | 3336 | WO2018071831 SEQ ID NO: 1047 |
| AAV variant | 3337 | WO2018071831 SEQ ID NO: 1048 |
| AAV variant | 3338 | WO2018071831 SEQ ID NO: 1049 |
| AAV variant | 3339 | WO2018071831 SEQ ID NO: 1050 |
| AAV variant | 3340 | WO2018071831 SEQ ID NO: 1051 |
| AAV variant | 3341 | WO2018071831 SEQ ID NO: 1052 |
| AAV variant | 3342 | WO2018071831 SEQ ID NO: 1053 |
| AAV variant | 3343 | WO2018071831 SEQ ID NO: 1054 |
| AAV variant | 3344 | WO2018071831 SEQ ID NO: 1055 |
| AAV variant | 3345 | WO2018071831 SEQ ID NO: 1056 |
| AAV variant | 3346 | WO2018071831 SEQ ID NO: 1057 |
| AAV variant | 3347 | WO2018071831 SEQ ID NO: 1058 |
| AAV variant | 3348 | WO2018071831 SEQ ID NO: 1059 |
| AAV variant | 3349 | WO2018071831 SEQ ID NO: 1060 |
| AAV variant | 3350 | WO2018071831 SEQ ID NO: 1061 |
| AAV variant | 3351 | WO2018071831 SEQ ID NO: 1062 |
| AAV variant | 3352 | WO2018071831 SEQ ID NO: 1063 |
| AAV variant | 3353 | WO2018071831 SEQ ID NO: 1064 |
| AAV variant | 3354 | WO2018071831 SEQ ID NO: 1065 |
| AAV variant | 3355 | WO2018071831 SEQ ID NO: 1066 |
| AAV variant | 3356 | WO2018071831 SEQ ID NO: 1067 |
| AAV variant | 3357 | WO2018071831 SEQ ID NO: 1068 |
| AAV variant | 3358 | WO2018071831 SEQ ID NO: 1069 |
| AAV variant | 3359 | WO2018071831 SEQ ID NO: 1070 |
| AAV variant | 3360 | WO2018071831 SEQ ID NO: 1071 |
| AAV variant | 3361 | WO2018071831 SEQ ID NO: 1072 |
| AAV variant | 3362 | WO2018071831 SEQ ID NO: 1073 |
| AAV variant | 3363 | WO2018071831 SEQ ID NO: 1074 |
| AAV variant | 3364 | WO2018071831 SEQ ID NO: 1075 |
| AAV variant | 3365 | WO2018071831 SEQ ID NO: 1076 |
| AAV variant | 3366 | WO2018071831 SEQ ID NO: 1077 |
| AAV variant | 3367 | WO2018071831 SEQ ID NO: 1078 |
| AAV variant | 3368 | WO2018071831 SEQ ID NO: 1079 |
| AAV variant | 3369 | WO2018071831 SEQ ID NO: 1080 |
| AAV variant | 3370 | WO2018071831 SEQ ID NO: 1081 |
| AAV variant | 3371 | WO2018071831 SEQ ID NO: 1082 |
| AAV variant | 3372 | WO2018071831 SEQ ID NO: 1083 |
| AAV variant | 3373 | WO2018071831 SEQ ID NO: 1084 |
| AAV variant | 3374 | WO2018071831 SEQ ID NO: 1085 |
| AAV variant | 3375 | WO2018071831 SEQ ID NO: 1086 |
| AAV variant | 3376 | WO2018071831 SEQ ID NO: 1087 |
| AAV variant | 3377 | WO2018071831 SEQ ID NO: 1088 |
| AAV variant | 3378 | WO2018071831 SEQ ID NO: 1089 |
| AAV variant | 3379 | WO2018071831 SEQ ID NO: 1090 |
| AAV variant | 3380 | WO2018071831 SEQ ID NO: 1091 |
| AAV variant | 3381 | WO2018071831 SEQ ID NO: 1092 |
| AAV variant | 3382 | WO2018071831 SEQ ID NO: 1093 |
| AAV variant | 3383 | WO2018071831 SEQ ID NO: 1094 |
| AAV variant | 3384 | WO2018071831 SEQ ID NO: 1095 |
| AAV variant | 3385 | WO2018071831 SEQ ID NO: 1096 |
| AAV variant | 3386 | WO2018071831 SEQ ID NO: 1097 |
| AAV variant | 3387 | WO2018071831 SEQ ID NO: 1098 |
| AAV variant | 3388 | WO2018071831 SEQ ID NO: 1099 |
| AAV variant | 3389 | WO2018071831 SEQ ID NO: 1100 |
| AAV variant | 3390 | WO2018071831 SEQ ID NO: 1101 |
| AAV variant | 3391 | WO2018071831 SEQ ID NO: 1102 |
| AAV variant | 3392 | WO2018071831 SEQ ID NO: 1103 |
| AAV variant | 3393 | WO2018071831 SEQ ID NO: 1104 |
| AAV variant | 3394 | WO2018071831 SEQ ID NO: 1105 |
| AAV variant | 3395 | WO2018071831 SEQ ID NO: 1106 |
| AAV variant | 3396 | WO2018071831 SEQ ID NO: 1107 |
| AAV variant | 3397 | WO2018071831 SEQ ID NO: 1108 |
| AAV variant | 3398 | WO2018071831 SEQ ID NO: 1109 |
| AAV variant | 3399 | WO2018071831 SEQ ID NO: 1110 |
| AAV variant | 3400 | WO2018071831 SEQ ID NO: 1111 |
| AAV variant | 3401 | WO2018071831 SEQ ID NO: 1112 |
| AAV variant | 3402 | WO2018071831 SEQ ID NO: 1113 |
| AAV variant | 3403 | WO2018071831 SEQ ID NO: 1114 |
| AAV variant | 3404 | WO2018071831 SEQ ID NO: 1115 |
| AAV variant | 3405 | WO2018071831 SEQ ID NO: 1116 |
| AAV variant | 3406 | WO2018071831 SEQ ID NO: 1117 |
| AAV variant | 3407 | WO2018071831 SEQ ID NO: 1118 |
| AAV variant | 3408 | WO2018071831 SEQ ID NO: 1119 |
| AAV variant | 3409 | WO2018071831 SEQ ID NO: 1120 |
| AAV variant | 3410 | WO2018071831 SEQ ID NO: 1121 |
| AAV variant | 3411 | WO2018071831 SEQ ID NO: 1122 |
| AAV variant | 3412 | WO2018071831 SEQ ID NO: 1123 |
| AAV variant | 3413 | WO2018071831 SEQ ID NO: 1124 |
| AAV variant | 3414 | WO2018071831 SEQ ID NO: 1125 |
| AAV variant | 3415 | WO2018071831 SEQ ID NO: 1126 |
| AAV variant | 3416 | WO2018071831 SEQ ID NO: 1127 |
| AAV variant | 3417 | WO2018071831 SEQ ID NO: 1128 |
| AAV variant | 3418 | WO2018071831 SEQ ID NO: 1129 |
| AAV variant | 3419 | WO2018071831 SEQ ID NO: 1130 |
| AAV variant | 3420 | WO2018071831 SEQ ID NO: 1131 |
| AAV variant | 3421 | WO2018071831 SEQ ID NO: 1132 |
| AAV variant | 3422 | WO2018071831 SEQ ID NO: 1133 |
| AAV variant | 3423 | WO2018071831 SEQ ID NO: 1134 |
| AAV variant | 3424 | WO2018071831 SEQ ID NO: 1135 |
| AAV variant | 3425 | WO2018071831 SEQ ID NO: 1136 |
| AAV variant | 3426 | WO2018071831 SEQ ID NO: 1137 |
| AAV variant | 3427 | WO2018071831 SEQ ID NO: 1138 |
| AAV variant | 3428 | WO2018071831 SEQ ID NO: 1139 |
| AAV variant | 3429 | WO2018071831 SEQ ID NO: 1140 |
| AAV variant | 3430 | WO2018071831 SEQ ID NO: 1141 |
| AAV variant | 3431 | WO2018071831 SEQ ID NO: 1142 |
| AAV variant | 3432 | WO2018071831 SEQ ID NO: 1143 |
| AAV variant | 3433 | WO2018071831 SEQ ID NO: 1144 |
| AAV variant | 3434 | WO2018071831 SEQ ID NO: 1145 |
| AAV variant | 3435 | WO2018071831 SEQ ID NO: 1146 |
| AAV variant | 3436 | WO2018071831 SEQ ID NO: 1147 |
| AAV variant | 3437 | WO2018071831 SEQ ID NO: 1148 |
| AAV variant | 3438 | WO2018071831 SEQ ID NO: 1149 |
| AAV variant | 3439 | WO2018071831 SEQ ID NO: 1150 |
| AAV variant | 3440 | WO2018071831 SEQ ID NO: 1151 |
| AAV variant | 3441 | WO2018071831 SEQ ID NO: 1152 |
| AAV variant | 3442 | WO2018071831 SEQ ID NO: 1153 |
| AAV variant | 3443 | WO2018071831 SEQ ID NO: 1154 |
| AAV variant | 3444 | WO2018071831 SEQ ID NO: 1155 |
| AAV variant | 3445 | WO2018071831 SEQ ID NO: 1156 |
| AAV variant | 3446 | WO2018071831 SEQ ID NO: 1157 |
| AAV variant | 3447 | WO2018071831 SEQ ID NO: 1158 |
| AAV variant | 3448 | WO2018071831 SEQ ID NO: 1159 |
| AAV variant | 3449 | WO2018071831 SEQ ID NO: 1160 |
| AAV variant | 3450 | WO2018071831 SEQ ID NO: 1161 |
| AAV variant | 3451 | WO2018071831 SEQ ID NO: 1162 |
| AAV variant | 3452 | WO2018071831 SEQ ID NO: 1163 |
| AAV variant | 3453 | WO2018071831 SEQ ID NO: 1164 |
| AAV variant | 3454 | WO2018071831 SEQ ID NO: 1165 |
| AAV variant | 3455 | WO2018071831 SEQ ID NO: 1166 |
| AAV variant | 3456 | WO2018071831 SEQ ID NO: 1167 |
| AAV variant | 3457 | WO2018071831 SEQ ID NO: 1168 |
| AAV variant | 3458 | WO2018071831 SEQ ID NO: 1169 |
| AAV variant | 3459 | WO2018071831 SEQ ID NO: 1170 |
| AAV variant | 3460 | WO2018071831 SEQ ID NO: 1171 |
| AAV variant | 3461 | WO2018071831 SEQ ID NO: 1172 |
| AAV variant | 3462 | WO2018071831 SEQ ID NO: 1173 |
| AAV variant | 3463 | WO2018071831 SEQ ID NO: 1174 |
| AAV variant | 3464 | WO2018071831 SEQ ID NO: 1175 |
| AAV variant | 3465 | WO2018071831 SEQ ID NO: 1176 |
| AAV variant | 3466 | WO2018071831 SEQ ID NO: 1177 |
| AAV variant | 3467 | WO2018071831 SEQ ID NO: 1178 |
| AAV variant | 3468 | WO2018071831 SEQ ID NO: 1179 |
| AAV variant | 3469 | WO2018071831 SEQ ID NO: 1180 |
| AAV variant | 3470 | WO2018071831 SEQ ID NO: 1181 |
| AAV variant | 3471 | WO2018071831 SEQ ID NO: 1182 |
| AAV variant | 3472 | WO2018071831 SEQ ID NO: 1183 |
| AAV variant | 3473 | WO2018071831 SEQ ID NO: 1184 |
| AAV variant | 3474 | WO2018071831 SEQ ID NO: 1185 |
| AAV variant | 3475 | WO2018071831 SEQ ID NO: 1186 |
| AAV variant | 3476 | WO2018071831 SEQ ID NO: 1187 |
| AAV variant | 3477 | WO2018071831 SEQ ID NO: 1188 |
| AAV variant | 3478 | WO2018071831 SEQ ID NO: 1189 |
| AAV variant | 3479 | WO2018071831 SEQ ID NO: 1190 |
| AAV variant | 3480 | WO2018071831 SEQ ID NO: 1191 |
| AAV variant | 3481 | WO2018071831 SEQ ID NO: 1192 |
| AAV variant | 3482 | WO2018071831 SEQ ID NO: 1193 |
| AAV variant | 3483 | WO2018071831 SEQ ID NO: 1194 |
| AAV variant | 3484 | WO2018071831 SEQ ID NO: 1195 |
| AAV variant | 3485 | WO2018071831 SEQ ID NO: 1196 |
| AAV variant | 3486 | WO2018071831 SEQ ID NO: 1197 |
| AAV variant | 3487 | WO2018071831 SEQ ID NO: 1198 |
| AAV variant | 3488 | WO2018071831 SEQ ID NO: 1199 |
| AAV variant | 3489 | WO2018071831 SEQ ID NO: 1200 |
| AAV variant | 3490 | WO2018071831 SEQ ID NO: 1201 |
| AAV variant | 3491 | WO2018071831 SEQ ID NO: 1202 |
| AAV variant | 3492 | WO2018071831 SEQ ID NO: 1203 |
| AAV variant | 3493 | WO2018071831 SEQ ID NO: 1204 |
| AAV variant | 3494 | WO2018071831 SEQ ID NO: 1205 |
| AAV variant | 3495 | WO2018071831 SEQ ID NO: 1206 |
| AAV variant | 3496 | WO2018071831 SEQ ID NO: 1207 |
| AAV variant | 3497 | WO2018071831 SEQ ID NO: 1208 |
| AAV variant | 3498 | WO2018071831 SEQ ID NO: 1209 |
| AAV variant | 3499 | WO2018071831 SEQ ID NO: 1210 |
| AAV variant | 3500 | WO2018071831 SEQ ID NO: 1211 |
| AAV variant | 3501 | WO2018071831 SEQ ID NO: 1212 |
| AAV variant | 3502 | WO2018071831 SEQ ID NO: 1213 |
| AAV variant | 3503 | WO2018071831 SEQ ID NO: 1214 |
| AAV variant | 3504 | WO2018071831 SEQ ID NO: 1215 |
| AAV variant | 3505 | WO2018071831 SEQ ID NO: 1216 |
| AAV variant | 3506 | WO2018071831 SEQ ID NO: 1217 |
| AAV variant | 3507 | WO2018071831 SEQ ID NO: 1218 |
| AAV variant | 3508 | WO2018071831 SEQ ID NO: 1219 |
| AAV variant | 3509 | WO2018071831 SEQ ID NO: 1220 |
| AAV variant | 3510 | WO2018071831 SEQ ID NO: 1221 |
| AAV variant | 3511 | WO2018071831 SEQ ID NO: 1222 |
| AAV variant | 3512 | WO2018071831 SEQ ID NO: 1223 |
| AAV variant | 3513 | WO2018071831 SEQ ID NO: 1224 |
| AAV variant | 3514 | WO2018071831 SEQ ID NO: 1225 |
| AAV variant | 3515 | WO2018071831 SEQ ID NO: 1226 |
| AAV variant | 3516 | WO2018071831 SEQ ID NO: 1227 |
| AAV variant | 3517 | WO2018071831 SEQ ID NO: 1228 |
| AAV variant | 3518 | WO2018071831 SEQ ID NO: 1229 |
| AAV variant | 3519 | WO2018071831 SEQ ID NO: 1230 |
| AAV variant | 3520 | WO2018071831 SEQ ID NO: 1231 |
| AAV variant | 3521 | WO2018071831 SEQ ID NO: 1232 |
| AAV variant | 3522 | WO2018071831 SEQ ID NO: 1233 |
| AAV variant | 3523 | WO2018071831 SEQ ID NO: 1234 |
| AAV variant | 3524 | WO2018071831 SEQ ID NO: 1235 |
| AAV variant | 3525 | WO2018071831 SEQ ID NO: 1236 |
| AAV variant | 3526 | WO2018071831 SEQ ID NO: 1237 |
| AAV variant | 3527 | WO2018071831 SEQ ID NO: 1238 |
| AAV variant | 3528 | WO2018071831 SEQ ID NO: 1239 |
| AAV variant | 3529 | WO2018071831 SEQ ID NO: 1240 |
| AAV variant | 3530 | WO2018071831 SEQ ID NO: 1241 |
| AAV variant | 3531 | WO2018071831 SEQ ID NO: 1242 |
| AAV variant | 3532 | WO2018071831 SEQ ID NO: 1243 |
| AAV variant | 3533 | WO2018071831 SEQ ID NO: 1244 |
| AAV variant | 3534 | WO2018071831 SEQ ID NO: 1245 |
| AAV variant | 3535 | WO2018071831 SEQ ID NO: 1246 |
| AAV variant | 3536 | WO2018071831 SEQ ID NO: 1247 |
| AAV variant | 3537 | WO2018071831 SEQ ID NO: 1248 |
| AAV variant | 3538 | WO2018071831 SEQ ID NO: 1249 |
| AAV variant | 3539 | WO2018071831 SEQ ID NO: 1250 |
| AAV variant | 3540 | WO2018071831 SEQ ID NO: 1251 |
| AAV variant | 3541 | WO2018071831 SEQ ID NO: 1252 |
| AAV variant | 3542 | WO2018071831 SEQ ID NO: 1253 |
| AAV variant | 3543 | WO2018071831 SEQ ID NO: 1254 |
| AAV variant | 3544 | WO2018071831 SEQ ID NO: 1255 |
| AAV variant | 3545 | WO2018071831 SEQ ID NO: 1256 |
| AAV variant | 3546 | WO2018071831 SEQ ID NO: 1257 |
| AAV variant | 3547 | WO2018071831 SEQ ID NO: 1258 |
| AAV variant | 3548 | WO2018071831 SEQ ID NO: 1259 |
| AAV variant | 3549 | WO2018071831 SEQ ID NO: 1260 |
| AAV variant | 3550 | WO2018071831 SEQ ID NO: 1261 |
| AAV variant | 3551 | WO2018071831 SEQ ID NO: 1262 |
| AAV variant | 3552 | WO2018071831 SEQ ID NO: 1263 |
| AAV variant | 3553 | WO2018071831 SEQ ID NO: 1264 |
| AAV variant | 3554 | WO2018071831 SEQ ID NO: 1265 |
| AAV variant | 3555 | WO2018071831 SEQ ID NO: 1266 |
| AAV variant | 3556 | WO2018071831 SEQ ID NO: 1267 |
| AAV variant | 3557 | WO2018071831 SEQ ID NO: 1268 |
| AAV variant | 3558 | WO2018071831 SEQ ID NO: 1269 |
| AAV variant | 3559 | WO2018071831 SEQ ID NO: 1270 |
| AAV variant | 3560 | WO2018071831 SEQ ID NO: 1271 |
| AAV variant | 3561 | WO2018071831 SEQ ID NO: 1272 |
| AAV variant | 3562 | WO2018071831 SEQ ID NO: 1273 |
| AAV variant | 3563 | WO2018071831 SEQ ID NO: 1274 |
| AAV variant | 3564 | WO2018071831 SEQ ID NO: 1275 |
| AAV variant | 3565 | WO2018071831 SEQ ID NO: 1276 |
| AAV variant | 3566 | WO2018071831 SEQ ID NO: 1277 |
| AAV variant | 3567 | WO2018071831 SEQ ID NO: 1278 |
| AAV variant | 3568 | WO2018071831 SEQ ID NO: 1279 |
| AAV variant | 3569 | WO2018071831 SEQ ID NO: 1280 |
| AAV variant | 3570 | WO2018071831 SEQ ID NO: 1281 |
| AAV variant | 3571 | WO2018071831 SEQ ID NO: 1282 |
| AAV variant | 3572 | WO2018071831 SEQ ID NO: 1283 |
| AAV variant | 3573 | WO2018071831 SEQ ID NO: 1284 |
| AAV variant | 3574 | WO2018071831 SEQ ID NO: 1285 |
| AAV variant | 3575 | WO2018071831 SEQ ID NO: 1286 |
| AAV variant | 3576 | WO2018071831 SEQ ID NO: 1287 |
| AAV variant | 3577 | WO2018071831 SEQ ID NO: 1288 |
| AAV variant | 3578 | WO2018071831 SEQ ID NO: 1289 |
| AAV variant | 3579 | WO2018071831 SEQ ID NO: 1290 |
| AAV variant | 3580 | WO2018071831 SEQ ID NO: 1291 |
| AAV variant | 3581 | WO2018071831 SEQ ID NO: 1292 |
| AAV variant | 3582 | WO2018071831 SEQ ID NO: 1293 |
| AAV variant | 3583 | WO2018071831 SEQ ID NO: 1294 |
| AAV variant | 3584 | WO2018071831 SEQ ID NO: 1295 |
| AAV variant | 3585 | WO2018071831 SEQ ID NO: 1296 |
| AAV variant | 3586 | WO2018071831 SEQ ID NO: 1297 |
| AAV variant | 3587 | WO2018071831 SEQ ID NO: 1298 |
| AAV variant | 3588 | WO2018071831 SEQ ID NO: 1299 |
| AAV variant | 3589 | WO2018071831 SEQ ID NO: 1300 |
| AAV variant | 3590 | WO2018071831 SEQ ID NO: 1301 |
| AAV variant | 3591 | WO2018071831 SEQ ID NO: 1302 |
| AAV variant | 3592 | WO2018071831 SEQ ID NO: 1303 |
| AAV variant | 3593 | WO2018071831 SEQ ID NO: 1304 |
| AAV variant | 3594 | WO2018071831 SEQ ID NO: 1305 |
| AAV variant | 3595 | WO2018071831 SEQ ID NO: 1306 |
| AAV variant | 3596 | WO2018071831 SEQ ID NO: 1307 |
| AAV variant | 3597 | WO2018071831 SEQ ID NO: 1308 |
| AAV variant | 3598 | WO2018071831 SEQ ID NO: 1309 |
| AAV variant | 3599 | WO2018071831 SEQ ID NO: 1310 |
| AAV variant | 3600 | WO2018071831 SEQ ID NO: 1311 |
| AAV variant | 3601 | WO2018071831 SEQ ID NO: 1312 |
| AAV variant | 3602 | WO2018071831 SEQ ID NO: 1313 |
| AAV variant | 3603 | WO2018071831 SEQ ID NO: 1314 |
| AAV variant | 3604 | WO2018071831 SEQ ID NO: 1315 |
| AAV variant | 3605 | WO2018071831 SEQ ID NO: 1316 |
| AAV variant | 3606 | WO2018071831 SEQ ID NO: 1317 |
| AAV variant | 3607 | WO2018071831 SEQ ID NO: 1318 |
| AAV variant | 3608 | WO2018071831 SEQ ID NO: 1319 |
| AAV variant | 3609 | WO2018071831 SEQ ID NO: 1320 |
| AAV variant | 3610 | WO2018071831 SEQ ID NO: 1321 |
| AAV variant | 3611 | WO2018071831 SEQ ID NO: 1322 |
| AAV variant | 3612 | WO2018071831 SEQ ID NO: 1323 |
| AAV variant | 3613 | WO2018071831 SEQ ID NO: 1324 |
| AAV variant | 3614 | WO2018071831 SEQ ID NO: 1325 |
| AAV variant | 3615 | WO2018071831 SEQ ID NO: 1326 |
| AAV variant | 3616 | WO2018071831 SEQ ID NO: 1327 |
| AAV variant | 3617 | WO2018071831 SEQ ID NO: 1328 |
| AAV variant | 3618 | WO2018071831 SEQ ID NO: 1329 |
| AAV variant | 3619 | WO2018071831 SEQ ID NO: 1330 |
| AAV variant | 3620 | WO2018071831 SEQ ID NO: 1331 |
| AAV variant | 3621 | WO2018071831 SEQ ID NO: 1332 |
| AAV variant | 3622 | WO2018071831 SEQ ID NO: 1333 |
| AAV variant | 3623 | WO2018071831 SEQ ID NO: 1334 |
| AAV variant | 3624 | WO2018071831 SEQ ID NO: 1335 |
| AAV variant | 3625 | WO2018071831 SEQ ID NO: 1336 |
| AAV variant | 3626 | WO2018071831 SEQ ID NO: 1337 |
| AAV variant | 3627 | WO2018071831 SEQ ID NO: 1338 |
| AAV variant | 3628 | WO2018071831 SEQ ID NO: 1339 |
| AAV variant | 3629 | WO2018071831 SEQ ID NO: 1340 |
| AAV variant | 3630 | WO2018071831 SEQ ID NO: 1341 |
| AAV variant | 3631 | WO2018071831 SEQ ID NO: 1342 |
| AAV variant | 3632 | WO2018071831 SEQ ID NO: 1343 |
| AAV variant | 3633 | WO2018071831 SEQ ID NO: 1344 |
| AAV variant | 3634 | WO2018071831 SEQ ID NO: 1345 |
| AAV variant | 3635 | WO2018071831 SEQ ID NO: 1346 |
| AAV variant | 3636 | WO2018071831 SEQ ID NO: 1347 |
| AAV variant | 3637 | WO2018071831 SEQ ID NO: 1348 |
| AAV variant | 3638 | WO2018071831 SEQ ID NO: 1349 |
| AAV variant | 3639 | WO2018071831 SEQ ID NO: 1350 |
| AAV variant | 3640 | WO2018071831 SEQ ID NO: 1351 |
| AAV variant | 3641 | WO2018071831 SEQ ID NO: 1352 |
| AAV variant | 3642 | WO2018071831 SEQ ID NO: 1353 |
| AAV variant | 3643 | WO2018071831 SEQ ID NO: 1354 |
| AAV variant | 3644 | WO2018071831 SEQ ID NO: 1355 |
| AAV variant | 3645 | WO2018071831 SEQ ID NO: 1356 |
| AAV variant | 3646 | WO2018071831 SEQ ID NO: 1357 |
| AAV variant | 3647 | WO2018071831 SEQ ID NO: 1358 |
| AAV variant | 3648 | WO2018071831 SEQ ID NO: 1359 |
| AAV variant | 3649 | WO2018071831 SEQ ID NO: 1360 |
| AAV variant | 3650 | WO2018071831 SEQ ID NO: 1361 |
| AAV variant | 3651 | WO2018071831 SEQ ID NO: 1362 |
| AAV variant | 3652 | WO2018071831 SEQ ID NO: 1363 |
| AAV variant | 3653 | WO2018071831 SEQ ID NO: 1364 |
| AAV variant | 3654 | WO2018071831 SEQ ID NO: 1365 |
| AAV variant | 3655 | WO2018071831 SEQ ID NO: 1366 |
| AAV variant | 3656 | WO2018071831 SEQ ID NO: 1367 |
| AAV variant | 3657 | WO2018071831 SEQ ID NO: 1368 |
| AAV variant | 3658 | WO2018071831 SEQ ID NO: 1369 |
| AAV variant | 3659 | WO2018071831 SEQ ID NO: 1370 |
| AAV variant | 3660 | WO2018071831 SEQ ID NO: 1371 |
| AAV variant | 3661 | WO2018071831 SEQ ID NO: 1372 |
| AAV variant | 3662 | WO2018071831 SEQ ID NO: 1373 |
| AAV variant | 3663 | WO2018071831 SEQ ID NO: 1374 |
| AAV variant | 3664 | WO2018071831 SEQ ID NO: 1375 |
| AAV variant | 3665 | WO2018071831 SEQ ID NO: 1376 |
| AAV variant | 3666 | WO2018071831 SEQ ID NO: 1377 |
| AAV variant | 3667 | WO2018071831 SEQ ID NO: 1378 |
| AAV variant | 3668 | WO2018071831 SEQ ID NO: 1379 |
| AAV variant | 3669 | WO2018071831 SEQ ID NO: 1380 |
| AAV variant | 3670 | WO2018071831 SEQ ID NO: 1381 |
| AAV variant | 3671 | WO2018071831 SEQ ID NO: 1382 |
| AAV variant | 3672 | WO2018071831 SEQ ID NO: 1383 |
| AAV variant | 3673 | WO2018071831 SEQ ID NO: 1384 |
| AAV variant | 3674 | WO2018071831 SEQ ID NO: 1385 |
| AAV variant | 3675 | WO2018071831 SEQ ID NO: 1386 |
| AAV variant | 3676 | WO2018071831 SEQ ID NO: 1387 |
| AAV variant | 3677 | WO2018071831 SEQ ID NO: 1388 |
| AAV variant | 3678 | WO2018071831 SEQ ID NO: 1389 |
| AAV variant | 3679 | WO2018071831 SEQ ID NO: 1390 |
| AAV variant | 3680 | WO2018071831 SEQ ID NO: 1391 |
| AAV variant | 3681 | WO2018071831 SEQ ID NO: 1392 |
| AAV variant | 3682 | WO2018071831 SEQ ID NO: 1393 |
| AAV variant | 3683 | WO2018071831 SEQ ID NO: 1394 |
| AAV variant | 3684 | WO2018071831 SEQ ID NO: 1395 |
| AAV variant | 3685 | WO2018071831 SEQ ID NO: 1396 |
| AAV variant | 3686 | WO2018071831 SEQ ID NO: 1397 |
| AAV variant | 3687 | WO2018071831 SEQ ID NO: 1398 |
| AAV variant | 3688 | WO2018071831 SEQ ID NO: 1399 |
| AAV variant | 3689 | WO2018071831 SEQ ID NO: 1400 |
| AAV variant | 3690 | WO2018071831 SEQ ID NO: 1401 |
| AAV variant | 3691 | WO2018071831 SEQ ID NO: 1402 |
| AAV variant | 3692 | WO2018071831 SEQ ID NO: 1403 |
| AAV variant | 3693 | WO2018071831 SEQ ID NO: 1404 |
| AAV variant | 3694 | WO2018071831 SEQ ID NO: 1405 |
| AAV variant | 3695 | WO2018071831 SEQ ID NO: 1406 |
| AAV variant | 3696 | WO2018071831 SEQ ID NO: 1407 |
| AAV variant | 3697 | WO2018071831 SEQ ID NO: 1408 |
| AAV variant | 3698 | WO2018071831 SEQ ID NO: 1409 |
| AAV variant | 3699 | WO2018071831 SEQ ID NO: 1410 |
| AAV variant | 3700 | WO2018071831 SEQ ID NO: 1411 |
| AAV variant | 3701 | WO2018071831 SEQ ID NO: 1412 |
| AAV variant | 3702 | WO2018071831 SEQ ID NO: 1413 |
| AAV variant | 3703 | WO2018071831 SEQ ID NO: 1414 |
| AAV variant | 3704 | WO2018071831 SEQ ID NO: 1415 |
| AAV variant | 3705 | WO2018071831 SEQ ID NO: 1416 |
| AAV variant | 3706 | WO2018071831 SEQ ID NO: 1417 |
| AAV variant | 3707 | WO2018071831 SEQ ID NO: 1418 |
| AAV variant | 3708 | WO2018071831 SEQ ID NO: 1419 |
| AAV variant | 3709 | WO2018071831 SEQ ID NO: 1420 |
| AAV variant | 3710 | WO2018071831 SEQ ID NO: 1421 |
| AAV variant | 3711 | WO2018071831 SEQ ID NO: 1422 |
| AAV variant | 3712 | WO2018071831 SEQ ID NO: 1423 |
| AAV variant | 3713 | WO2018071831 SEQ ID NO: 1424 |
| AAV variant | 3714 | WO2018071831 SEQ ID NO: 1425 |
| AAV variant | 3715 | WO2018071831 SEQ ID NO: 1426 |
| AAV variant | 3716 | WO2018071831 SEQ ID NO: 1427 |
| AAV variant | 3717 | WO2018071831 SEQ ID NO: 1428 |
| AAV variant | 3718 | WO2018071831 SEQ ID NO: 1429 |
| AAV variant | 3719 | WO2018071831 SEQ ID NO: 1430 |
| AAV variant | 3720 | WO2018071831 SEQ ID NO: 1431 |
| AAV variant | 3721 | WO2018071831 SEQ ID NO: 1432 |
| AAV variant | 3722 | WO2018071831 SEQ ID NO: 1433 |
| AAV variant | 3723 | WO2018071831 SEQ ID NO: 1434 |
| AAV variant | 3724 | WO2018071831 SEQ ID NO: 1435 |
| AAV variant | 3725 | WO2018071831 SEQ ID NO: 1436 |
| AAV variant | 3726 | WO2018071831 SEQ ID NO: 1437 |
| AAV variant | 3727 | WO2018071831 SEQ ID NO: 1438 |
| AAV variant | 3728 | WO2018071831 SEQ ID NO: 1439 |
| AAV variant | 3729 | WO2018071831 SEQ ID NO: 1440 |
| AAV variant | 3730 | WO2018071831 SEQ ID NO: 1441 |
| AAV variant | 3731 | WO2018071831 SEQ ID NO: 1442 |
| AAV variant | 3732 | WO2018071831 SEQ ID NO: 1443 |
| AAV variant | 3733 | WO2018071831 SEQ ID NO: 1444 |
| AAV variant | 3734 | WO2018071831 SEQ ID NO: 1445 |
| AAV variant | 3735 | WO2018071831 SEQ ID NO: 1446 |
| AAV variant | 3736 | WO2018071831 SEQ ID NO: 1447 |
| AAV variant | 3737 | WO2018071831 SEQ ID NO: 1448 |
| AAV variant | 3738 | WO2018071831 SEQ ID NO: 1449 |
| AAV variant | 3739 | WO2018071831 SEQ ID NO: 1450 |
| AAV variant | 3740 | WO2018071831 SEQ ID NO: 1451 |
| AAV variant | 3741 | WO2018071831 SEQ ID NO: 1452 |
| AAV variant | 3742 | WO2018071831 SEQ ID NO: 1453 |
| AAV variant | 3743 | WO2018071831 SEQ ID NO: 1454 |
| AAV variant | 3744 | WO2018071831 SEQ ID NO: 1455 |
| AAV variant | 3745 | WO2018071831 SEQ ID NO: 1456 |
| AAV variant | 3746 | WO2018071831 SEQ ID NO: 1457 |
| AAV variant | 3747 | WO2018071831 SEQ ID NO: 1458 |
| AAV variant | 3748 | WO2018071831 SEQ ID NO: 1459 |
| AAV variant | 3749 | WO2018071831 SEQ ID NO: 1460 |
| AAV variant | 3750 | WO2018071831 SEQ ID NO: 1461 |
| AAV variant | 3751 | WO2018071831 SEQ ID NO: 1462 |
| AAV variant | 3752 | WO2018071831 SEQ ID NO: 1463 |
| AAV variant | 3753 | WO2018071831 SEQ ID NO: 1464 |
| AAV variant | 3754 | WO2018071831 SEQ ID NO: 1465 |
| AAV variant | 3755 | WO2018071831 SEQ ID NO: 1466 |
| AAV variant | 3756 | WO2018071831 SEQ ID NO: 1467 |
| AAV variant | 3757 | WO2018071831 SEQ ID NO: 1468 |
| AAV variant | 3758 | WO2018071831 SEQ ID NO: 1469 |
| AAV variant | 3759 | WO2018071831 SEQ ID NO: 1470 |
| AAV variant | 3760 | WO2018071831 SEQ ID NO: 1471 |
| AAV variant | 3761 | WO2018071831 SEQ ID NO: 1472 |
| AAV variant | 3762 | WO2018071831 SEQ ID NO: 1473 |
| AAV variant | 3763 | WO2018071831 SEQ ID NO: 1474 |
| AAV variant | 3764 | WO2018071831 SEQ ID NO: 1475 |
| AAV variant | 3765 | WO2018071831 SEQ ID NO: 1476 |
| AAV variant | 3766 | WO2018071831 SEQ ID NO: 1477 |
| AAV variant | 3767 | WO2018071831 SEQ ID NO: 1478 |
| AAV variant | 3768 | WO2018071831 SEQ ID NO: 1479 |
| AAV variant | 3769 | WO2018071831 SEQ ID NO: 1480 |
| AAV variant | 3770 | WO2018071831 SEQ ID NO: 1481 |
| AAV variant | 3771 | WO2018071831 SEQ ID NO: 1482 |
| AAV variant | 3772 | WO2018071831 SEQ ID NO: 1483 |
| AAV variant | 3773 | WO2018071831 SEQ ID NO: 1484 |
| AAV variant | 3774 | WO2018071831 SEQ ID NO: 1485 |
| AAV variant | 3775 | WO2018071831 SEQ ID NO: 1486 |
| AAV variant | 3776 | WO2018071831 SEQ ID NO: 1487 |
| AAV variant | 3777 | WO2018071831 SEQ ID NO: 1488 |
| AAV variant | 3778 | WO2018071831 SEQ ID NO: 1489 |
| AAV variant | 3779 | WO2018071831 SEQ ID NO: 1490 |
| AAV variant | 3780 | WO2018071831 SEQ ID NO: 1491 |
| AAV variant | 3781 | WO2018071831 SEQ ID NO: 1492 |
| AAV variant | 3782 | WO2018071831 SEQ ID NO: 1493 |
| AAV variant | 3783 | WO2018071831 SEQ ID NO: 1494 |
| AAV variant | 3784 | WO2018071831 SEQ ID NO: 1495 |
| AAV variant | 3785 | WO2018071831 SEQ ID NO: 1496 |
| AAV variant | 3786 | WO2018071831 SEQ ID NO: 1497 |
| AAV variant | 3787 | WO2018071831 SEQ ID NO: 1498 |
| AAV variant | 3788 | WO2018071831 SEQ ID NO: 1499 |
| AAV variant | 3789 | WO2018071831 SEQ ID NO: 1500 |
| AAV variant | 3790 | WO2018071831 SEQ ID NO: 1501 |
| AAV variant | 3791 | WO2018071831 SEQ ID NO: 1502 |
| AAV variant | 3792 | WO2018071831 SEQ ID NO: 1503 |
| AAV variant | 3793 | WO2018071831 SEQ ID NO: 1504 |
| AAV variant | 3794 | WO2018071831 SEQ ID NO: 1505 |
| AAV variant | 3795 | WO2018071831 SEQ ID NO: 1506 |
| AAV variant | 3796 | WO2018071831 SEQ ID NO: 1507 |
| AAV variant | 3797 | WO2018071831 SEQ ID NO: 1508 |
| AAV variant | 3798 | WO2018071831 SEQ ID NO: 1509 |
| AAV variant | 3799 | WO2018071831 SEQ ID NO: 1510 |
| AAV variant | 3800 | WO2018071831 SEQ ID NO: 1511 |
| AAV variant | 3801 | WO2018071831 SEQ ID NO: 1512 |
| AAV variant | 3802 | WO2018071831 SEQ ID NO: 1513 |
| AAV variant | 3803 | WO2018071831 SEQ ID NO: 1514 |
| AAV variant | 3804 | WO2018071831 SEQ ID NO: 1515 |
| AAV variant | 3805 | WO2018071831 SEQ ID NO: 1516 |
| AAV variant | 3806 | WO2018071831 SEQ ID NO: 1517 |
| AAV variant | 3807 | WO2018071831 SEQ ID NO: 1518 |
| AAV variant | 3808 | WO2018071831 SEQ ID NO: 1519 |
| AAV variant | 3809 | WO2018071831 SEQ ID NO: 1520 |
| AAV variant | 3810 | WO2018071831 SEQ ID NO: 1521 |
| AAV variant | 3811 | WO2018071831 SEQ ID NO: 1522 |
| AAV variant | 3812 | WO2018071831 SEQ ID NO: 1523 |
| AAV variant | 3813 | WO2018071831 SEQ ID NO: 1524 |
| AAV variant | 3814 | WO2018071831 SEQ ID NO: 1525 |
| AAV variant | 3815 | WO2018071831 SEQ ID NO: 1526 |
| AAV variant | 3816 | WO2018071831 SEQ ID NO: 1527 |
| AAV variant | 3817 | WO2018071831 SEQ ID NO: 1528 |
| AAV variant | 3818 | WO2018071831 SEQ ID NO: 1529 |
| AAV variant | 3819 | WO2018071831 SEQ ID NO: 1530 |
| AAV variant | 3820 | WO2018071831 SEQ ID NO: 1531 |
| AAV variant | 3821 | WO2018071831 SEQ ID NO: 1532 |
| AAV variant | 3822 | WO2018071831 SEQ ID NO: 1533 |
| AAV variant | 3823 | WO2018071831 SEQ ID NO: 1534 |
| AAV variant | 3824 | WO2018071831 SEQ ID NO: 1535 |
| AAV variant | 3825 | WO2018071831 SEQ ID NO: 1536 |
| AAV variant | 3826 | WO2018071831 SEQ ID NO: 1537 |
| AAV variant | 3827 | WO2018071831 SEQ ID NO: 1538 |
| AAV variant | 3828 | WO2018071831 SEQ ID NO: 1539 |
| AAV variant | 3829 | WO2018071831 SEQ ID NO: 1540 |
| AAV variant | 3830 | WO2018071831 SEQ ID NO: 1541 |
| AAV variant | 3831 | WO2018071831 SEQ ID NO: 1542 |
| AAV variant | 3832 | WO2018071831 SEQ ID NO: 1543 |
| AAV variant | 3833 | WO2018071831 SEQ ID NO: 1544 |
| AAV variant | 3834 | WO2018071831 SEQ ID NO: 1545 |
| AAV variant | 3835 | WO2018071831 SEQ ID NO: 1546 |
| AAV variant | 3836 | WO2018071831 SEQ ID NO: 1547 |
| AAV variant | 3837 | WO2018071831 SEQ ID NO: 1548 |
| AAV variant | 3838 | WO2018071831 SEQ ID NO: 1549 |
| AAV variant | 3839 | WO2018071831 SEQ ID NO: 1550 |
| AAV variant | 3840 | WO2018071831 SEQ ID NO: 1551 |
| AAV variant | 3841 | WO2018071831 SEQ ID NO: 1552 |
| AAV variant | 3842 | WO2018071831 SEQ ID NO: 1553 |
| AAV variant | 3843 | WO2018071831 SEQ ID NO: 1554 |
| AAV variant | 3844 | WO2018071831 SEQ ID NO: 1555 |
| AAV variant | 3845 | WO2018071831 SEQ ID NO: 1556 |
| AAV variant | 3846 | WO2018071831 SEQ ID NO: 1557 |
| AAV variant | 3847 | WO2018071831 SEQ ID NO: 1558 |
| AAV variant | 3848 | WO2018071831 SEQ ID NO: 1559 |
| AAV variant | 3849 | WO2018071831 SEQ ID NO: 1560 |
| AAV variant | 3850 | WO2018071831 SEQ ID NO: 1561 |
| AAV variant | 3851 | WO2018071831 SEQ ID NO: 1562 |
| AAV variant | 3852 | WO2018071831 SEQ ID NO: 1563 |
| AAV variant | 3853 | WO2018071831 SEQ ID NO: 1564 |
| AAV variant | 3854 | WO2018071831 SEQ ID NO: 1565 |
| AAV variant | 3855 | WO2018071831 SEQ ID NO: 1566 |
| AAV variant | 3856 | WO2018071831 SEQ ID NO: 1567 |
| AAV variant | 3857 | WO2018071831 SEQ ID NO: 1568 |
| AAV variant | 3858 | WO2018071831 SEQ ID NO: 1569 |
| AAV variant | 3859 | WO2018071831 SEQ ID NO: 1570 |
| AAV variant | 3860 | WO2018071831 SEQ ID NO: 1571 |
| AAV variant | 3861 | WO2018071831 SEQ ID NO: 1572 |
| AAV variant | 3862 | WO2018071831 SEQ ID NO: 1573 |
| AAV variant | 3863 | WO2018071831 SEQ ID NO: 1574 |
| AAV variant | 3864 | WO2018071831 SEQ ID NO: 1575 |
| AAV variant | 3865 | WO2018071831 SEQ ID NO: 1576 |
| AAV variant | 3866 | WO2018071831 SEQ ID NO: 1577 |
| AAV variant | 3867 | WO2018071831 SEQ ID NO: 1578 |
| AAV variant | 3868 | WO2018071831 SEQ ID NO: 1579 |
| AAV variant | 3869 | WO2018071831 SEQ ID NO: 1580 |
| AAV variant | 3870 | WO2018071831 SEQ ID NO: 1581 |
| AAV variant | 3871 | WO2018071831 SEQ ID NO: 1582 |
| AAV variant | 3872 | WO2018071831 SEQ ID NO: 1583 |
| AAV variant | 3873 | WO2018071831 SEQ ID NO: 1584 |
| AAV variant | 3874 | WO2018071831 SEQ ID NO: 1585 |
| AAV variant | 3875 | WO2018071831 SEQ ID NO: 1586 |
| AAV variant | 3876 | WO2018071831 SEQ ID NO: 1587 |
| AAV variant | 3877 | WO2018071831 SEQ ID NO: 1588 |
| AAV variant | 3878 | WO2018071831 SEQ ID NO: 1589 |
| AAV variant | 3879 | WO2018071831 SEQ ID NO: 1590 |
| AAV variant | 3880 | WO2018071831 SEQ ID NO: 1591 |
| AAV variant | 3881 | WO2018071831 SEQ ID NO: 1592 |
| AAV variant | 3882 | WO2018071831 SEQ ID NO: 1593 |
| AAV variant | 3883 | WO2018071831 SEQ ID NO: 1594 |
| AAV variant | 3884 | WO2018071831 SEQ ID NO: 1595 |
| AAV variant | 3885 | WO2018071831 SEQ ID NO: 1596 |
| AAV variant | 3886 | WO2018071831 SEQ ID NO: 1597 |
| AAV variant | 3887 | WO2018071831 SEQ ID NO: 1598 |
| AAV variant | 3888 | WO2018071831 SEQ ID NO: 1599 |
| AAV variant | 3889 | WO2018071831 SEQ ID NO: 1600 |
| AAV variant | 3890 | WO2018071831 SEQ ID NO: 1601 |
| AAV variant | 3891 | WO2018071831 SEQ ID NO: 1602 |
| AAV variant | 3892 | WO2018071831 SEQ ID NO: 1603 |
| AAV variant | 3893 | WO2018071831 SEQ ID NO: 1604 |
| AAV variant | 3894 | WO2018071831 SEQ ID NO: 1605 |
| AAV variant | 3895 | WO2018071831 SEQ ID NO: 1606 |
| AAV variant | 3896 | WO2018071831 SEQ ID NO: 1607 |
| AAV variant | 3897 | WO2018071831 SEQ ID NO: 1608 |
| AAV variant | 3898 | WO2018071831 SEQ ID NO: 1609 |
| AAV variant | 3899 | WO2018071831 SEQ ID NO: 1610 |
| AAV variant | 3900 | WO2018071831 SEQ ID NO: 1611 |
| AAV variant | 3901 | WO2018071831 SEQ ID NO: 1612 |
| AAV variant | 3902 | WO2018071831 SEQ ID NO: 1613 |
| AAV variant | 3903 | WO2018071831 SEQ ID NO: 1614 |
| AAV variant | 3904 | WO2018071831 SEQ ID NO: 1615 |
| AAV variant | 3905 | WO2018071831 SEQ ID NO: 1616 |
| AAV variant | 3906 | WO2018071831 SEQ ID NO: 1617 |
| AAV variant | 3907 | WO2018071831 SEQ ID NO: 1618 |
| AAV variant | 3908 | WO2018071831 SEQ ID NO: 1619 |
| AAV variant | 3909 | WO2018071831 SEQ ID NO: 1620 |
| AAV variant | 3910 | WO2018071831 SEQ ID NO: 1621 |
| AAV variant | 3911 | WO2018071831 SEQ ID NO: 1622 |
| AAV variant | 3912 | WO2018071831 SEQ ID NO: 1623 |
| AAV variant | 3913 | WO2018071831 SEQ ID NO: 1624 |
| AAV variant | 3914 | WO2018071831 SEQ ID NO: 1625 |
| AAV variant | 3915 | WO2018071831 SEQ ID NO: 1626 |
| AAV variant | 3916 | WO2018071831 SEQ ID NO: 1627 |
| AAV variant | 3917 | WO2018071831 SEQ ID NO: 1628 |
| AAV variant | 3918 | WO2018071831 SEQ ID NO: 1629 |
| AAV variant | 3919 | WO2018071831 SEQ ID NO: 1630 |
| AAV variant | 3920 | WO2018071831 SEQ ID NO: 1631 |
| AAV variant | 3921 | WO2018071831 SEQ ID NO: 1632 |
| AAV variant | 3922 | WO2018071831 SEQ ID NO: 1633 |
| AAV variant | 3923 | WO2018071831 SEQ ID NO: 1634 |
| AAV variant | 3924 | WO2018071831 SEQ ID NO: 1635 |
| AAV variant | 3925 | WO2018071831 SEQ ID NO: 1636 |
| AAV variant | 3926 | WO2018071831 SEQ ID NO: 1637 |
| AAV variant | 3927 | WO2018071831 SEQ ID NO: 1638 |
| AAV variant | 3928 | WO2018071831 SEQ ID NO: 1639 |
| AAV variant | 3929 | WO2018071831 SEQ ID NO: 1640 |
| AAV variant | 3930 | WO2018071831 SEQ ID NO: 1641 |
| AAV variant | 3931 | WO2018071831 SEQ ID NO: 1642 |
| AAV variant | 3932 | WO2018071831 SEQ ID NO: 1643 |
| AAV variant | 3933 | WO2018071831 SEQ ID NO: 1644 |
| AAV variant | 3934 | WO2018071831 SEQ ID NO: 1645 |
| AAV variant | 3935 | WO2018071831 SEQ ID NO: 1646 |
| AAV variant | 3936 | WO2018071831 SEQ ID NO: 1647 |
| AAV variant | 3937 | WO2018071831 SEQ ID NO: 1648 |
| AAV variant | 3938 | WO2018071831 SEQ ID NO: 1649 |
| AAV variant | 3939 | WO2018071831 SEQ ID NO: 1650 |
| AAV variant | 3940 | WO2018071831 SEQ ID NO: 1651 |
| AAV variant | 3941 | WO2018071831 SEQ ID NO: 1652 |
| AAV variant | 3942 | WO2018071831 SEQ ID NO: 1653 |
| AAV variant | 3943 | WO2018071831 SEQ ID NO: 1654 |
| AAV variant | 3944 | WO2018071831 SEQ ID NO: 1655 |
| AAV variant | 3945 | WO2018071831 SEQ ID NO: 1656 |
| AAV variant | 3946 | WO2018071831 SEQ ID NO: 1657 |
| AAV variant | 3947 | WO2018071831 SEQ ID NO: 1658 |
| AAV variant | 3948 | WO2018071831 SEQ ID NO: 1659 |
| AAV variant | 3949 | WO2018071831 SEQ ID NO: 1660 |
| AAV variant | 3950 | WO2018071831 SEQ ID NO: 1661 |
| AAV variant | 3951 | WO2018071831 SEQ ID NO: 1662 |
| AAV variant | 3952 | WO2018071831 SEQ ID NO: 1663 |
| AAV variant | 3953 | WO2018071831 SEQ ID NO: 1664 |
| AAV variant | 3954 | WO2018071831 SEQ ID NO: 1665 |
| AAV variant | 3955 | WO2018071831 SEQ ID NO: 1666 |
| AAV variant | 3956 | WO2018071831 SEQ ID NO: 1667 |
| AAV variant | 3957 | WO2018071831 SEQ ID NO: 1668 |
| AAV variant | 3958 | WO2018071831 SEQ ID NO: 1669 |
| AAV variant | 3959 | WO2018071831 SEQ ID NO: 1670 |
| AAV variant | 3960 | WO2018071831 SEQ ID NO: 1671 |
| AAV variant | 3961 | WO2018071831 SEQ ID NO: 1672 |
| AAV variant | 3962 | WO2018071831 SEQ ID NO: 1673 |
| AAV variant | 3963 | WO2018071831 SEQ ID NO: 1674 |
| AAV variant | 3964 | WO2018071831 SEQ ID NO: 1675 |
| AAV variant | 3965 | WO2018071831 SEQ ID NO: 1676 |
| AAV variant | 3966 | WO2018071831 SEQ ID NO: 1677 |
| AAV variant | 3967 | WO2018071831 SEQ ID NO: 1678 |
| AAV variant | 3968 | WO2018071831 SEQ ID NO: 1679 |
| AAV variant | 3969 | WO2018071831 SEQ ID NO: 1680 |
| AAV variant | 3970 | WO2018071831 SEQ ID NO: 1681 |
| AAV variant | 3971 | WO2018071831 SEQ ID NO: 1682 |
| AAV variant | 3972 | WO2018071831 SEQ ID NO: 1683 |
| AAV variant | 3973 | WO2018071831 SEQ ID NO: 1684 |
| AAV variant | 3974 | WO2018071831 SEQ ID NO: 1685 |
| AAV variant | 3975 | WO2018071831 SEQ ID NO: 1686 |
| AAV variant | 3976 | WO2018071831 SEQ ID NO: 1687 |
| AAV variant | 3977 | WO2018071831 SEQ ID NO: 1688 |
| AAV variant | 3978 | WO2018071831 SEQ ID NO: 1689 |
| AAV variant | 3979 | WO2018071831 SEQ ID NO: 1690 |
| AAV variant | 3980 | WO2018071831 SEQ ID NO: 1691 |
| AAV variant | 3981 | WO2018071831 SEQ ID NO: 1692 |
| AAV variant | 3982 | WO2018071831 SEQ ID NO: 1693 |
| AAV variant | 3983 | WO2018071831 SEQ ID NO: 1694 |
| AAV variant | 3984 | WO2018071831 SEQ ID NO: 1695 |
| AAV variant | 3985 | WO2018071831 SEQ ID NO: 1696 |
| AAV variant | 3986 | WO2018071831 SEQ ID NO: 1697 |
| AAV variant | 3987 | WO2018071831 SEQ ID NO: 1698 |
| AAV variant | 3988 | WO2018071831 SEQ ID NO: 1699 |
| AAV variant | 3989 | WO2018071831 SEQ ID NO: 1700 |
| AAV variant | 3990 | WO2018071831 SEQ ID NO: 1701 |
| AAV variant | 3991 | WO2018071831 SEQ ID NO: 1702 |
| AAV variant | 3992 | WO2018071831 SEQ ID NO: 1703 |
| AAV variant | 3993 | WO2018071831 SEQ ID NO: 1704 |
| AAV variant | 3994 | WO2018071831 SEQ ID NO: 1705 |
| AAV variant | 3995 | WO2018071831 SEQ ID NO: 1706 |
| AAV variant | 3996 | WO2018071831 SEQ ID NO: 1707 |
| AAV variant | 3997 | WO2018071831 SEQ ID NO: 1708 |
| AAV variant | 3998 | WO2018071831 SEQ ID NO: 1709 |
| AAV variant | 3999 | WO2018071831 SEQ ID NO: 1710 |
| AAV variant | 4000 | WO2018071831 SEQ ID NO: 1711 |
| AAV variant | 4001 | WO2018071831 SEQ ID NO: 1712 |
| AAV variant | 4002 | WO2018071831 SEQ ID NO: 1713 |
| AAV variant | 4003 | WO2018071831 SEQ ID NO: 1714 |
| AAV variant | 4004 | WO2018071831 SEQ ID NO: 1715 |
| AAV variant | 4005 | WO2018071831 SEQ ID NO: 1716 |
| AAV variant | 4006 | WO2018071831 SEQ ID NO: 1717 |
| AAV variant | 4007 | WO2018071831 SEQ ID NO: 1718 |
| AAV2 variant | 4008 | WO2018071831 SEQ ID NO: 1726 |
| AAV2 variant | 4009 | WO2018071831 SEQ ID NO: 1727 |
| AAV2 variant | 4010 | WO2018071831 SEQ ID NO: 1728 |
| AAV2 variant | 4011 | WO2018071831 SEQ ID NO: 1729 |
| AAV2 variant | 4012 | WO2018071831 SEQ ID NO: 1730 |
| AAV2 variant | 4013 | WO2018071831 SEQ ID NO: 1731 |
| AAV2 variant | 4014 | WO2018071831 SEQ ID NO: 1732 |
| AAV2 variant | 4015 | WO2018071831 SEQ ID NO: 1733 |
| AAV2 variant | 4016 | WO2018071831 SEQ ID NO: 1734 |
| AAV2 variant | 4017 | WO2018071831 SEQ ID NO: 1735 |
| AAV2 variant | 4018 | WO2018071831 SEQ ID NO: 1736 |
| AAV2 variant | 4019 | WO2018071831 SEQ ID NO: 1737 |
| AAV2 variant | 4020 | WO2018071831 SEQ ID NO: 1738 |
| AAV2 variant | 4021 | WO2018071831 SEQ ID NO: 1739 |
| AAV2 variant | 4022 | WO2018071831 SEQ ID NO: 1740 |
| AAV2 variant | 4023 | WO2018071831 SEQ ID NO: 1741 |
| AAV2 variant | 4024 | WO2018071831 SEQ ID NO: 1742 |
| AAV2 variant | 4025 | WO2018071831 SEQ ID NO: 1743 |
| AAV2 variant | 4026 | WO2018071831 SEQ ID NO: 1744 |
| AAV2 variant | 4027 | WO2018071831 SEQ ID NO: 1745 |
| AAV2 variant | 4028 | WO2018071831 SEQ ID NO: 1746 |
| AAV2 variant | 4029 | WO2018071831 SEQ ID NO: 1747 |
| AAV2 variant | 4030 | WO2018071831 SEQ ID NO: 1748 |
| AAV2 variant | 4031 | WO2018071831 SEQ ID NO: 1749 |
| AAV2 variant | 4032 | WO2018071831 SEQ ID NO: 1750 |
| AAV2 variant | 4033 | WO2018071831 SEQ ID NO: 1751 |
| AAV2 variant | 4034 | WO2018071831 SEQ ID NO: 1752 |
| AAV2 variant | 4035 | WO2018071831 SEQ ID NO: 1753 |
| AAV2 variant | 4036 | WO2018071831 SEQ ID NO: 1754 |
| AAV2 variant | 4037 | WO2018071831 SEQ ID NO: 1755 |
| AAV2 variant | 4038 | WO2018071831 SEQ ID NO: 1756 |
| AAV2 variant | 4039 | WO2018071831 SEQ ID NO: 1757 |
| AAV2 variant | 4040 | WO2018071831 SEQ ID NO: 1758 |
| AAV2 variant | 4041 | WO2018071831 SEQ ID NO: 1759 |
| AAV2 variant | 4042 | WO2018071831 SEQ ID NO: 1760 |
| AAV2 variant | 4043 | WO2018071831 SEQ ID NO: 1761 |
| AAV2 variant | 4044 | WO2018071831 SEQ ID NO: 1762 |
| AAV2 variant | 4045 | WO2018071831 SEQ ID NO: 1763 |
| AAV2 variant | 4046 | WO2018071831 SEQ ID NO: 1764 |
| AAV2 variant | 4047 | WO2018071831 SEQ ID NO: 1765 |
| AAV2 variant | 4048 | WO2018071831 SEQ ID NO: 1766 |
| AAV2 variant | 4049 | WO2018071831 SEQ ID NO: 1767 |
| AAV2 variant | 4050 | WO2018071831 SEQ ID NO: 1768 |
| AAV2 variant | 4051 | WO2018071831 SEQ ID NO: 1769 |
| AAV2 variant | 4052 | WO2018071831 SEQ ID NO: 1770 |
| AAV2 variant | 4053 | WO2018071831 SEQ ID NO: 1771 |
| AAV2 variant | 4054 | WO2018071831 SEQ ID NO: 1772 |
| AAV2 variant | 4055 | WO2018071831 SEQ ID NO: 1773 |
| AAV2 variant | 4056 | WO2018071831 SEQ ID NO: 1774 |
| AAV2 variant | 4057 | WO2018071831 SEQ ID NO: 1775 |
| AAV2 variant | 4058 | WO2018071831 SEQ ID NO: 1776 |
| AAV2 variant | 4059 | WO2018071831 SEQ ID NO: 1777 |
| AAV2 variant | 4060 | WO2018071831 SEQ ID NO: 1778 |
| AAV2 variant | 4061 | WO2018071831 SEQ ID NO: 1779 |
| AAV2 variant | 4062 | WO2018071831 SEQ ID NO: 1780 |
| AAV2 variant | 4063 | WO2018071831 SEQ ID NO: 1781 |
| AAV2 variant | 4064 | WO2018071831 SEQ ID NO: 1782 |
| AAV2 variant | 4065 | WO2018071831 SEQ ID NO: 1783 |
| AAV2 variant | 4066 | WO2018071831 SEQ ID NO: 1784 |
| AAV2 variant | 4067 | WO2018071831 SEQ ID NO: 1785 |
| AAV2 variant | 4068 | WO2018071831 SEQ ID NO: 1786 |
| AAV2 variant | 4069 | WO2018071831 SEQ ID NO: 1787 |
| AAV2 variant | 4070 | WO2018071831 SEQ ID NO: 1788 |
| AAV2 variant | 4071 | WO2018071831 SEQ ID NO: 1789 |
| AAV2 variant | 4072 | WO2018071831 SEQ ID NO: 1790 |
| AAV2 variant | 4073 | WO2018071831 SEQ ID NO: 1791 |
| AAV2 variant | 4074 | WO2018071831 SEQ ID NO: 1792 |
| AAV2 variant | 4075 | WO2018071831 SEQ ID NO: 1793 |
| AAV2 variant | 4076 | WO2018071831 SEQ ID NO: 1794 |
| AAV2 variant | 4077 | WO2018071831 SEQ ID NO: 1795 |
| AAV2 variant | 4078 | WO2018071831 SEQ ID NO: 1796 |
| AAV2 variant | 4079 | WO2018071831 SEQ ID NO: 1797 |
| AAV2 variant | 4080 | WO2018071831 SEQ ID NO: 1798 |
| AAV2 variant | 4081 | WO2018071831 SEQ ID NO: 1799 |
| AAV2 variant | 4082 | WO2018071831 SEQ ID NO: 1800 |
| AAV2 variant | 4083 | WO2018071831 SEQ ID NO: 1801 |
| AAV2 variant | 4084 | WO2018071831 SEQ ID NO: 1802 |
| AAV2 variant | 4085 | WO2018071831 SEQ ID NO: 1803 |
| AAV2 variant | 4086 | WO2018071831 SEQ ID NO: 1804 |
| AAV2 variant | 4087 | WO2018071831 SEQ ID NO: 1805 |
| AAV2 variant | 4088 | WO2018071831 SEQ ID NO: 1806 |
| AAV2 variant | 4089 | WO2018071831 SEQ ID NO: 1807 |
| AAV2 variant | 4090 | WO2018071831 SEQ ID NO: 1808 |
| AAV2 variant | 4091 | WO2018071831 SEQ ID NO: 1809 |
| AAV2 variant | 4092 | WO2018071831 SEQ ID NO: 1810 |
| AAV2 variant | 4093 | WO2018071831 SEQ ID NO: 1811 |
| AAV2 variant | 4094 | WO2018071831 SEQ ID NO: 1812 |
| AAV2 variant | 4095 | WO2018071831 SEQ ID NO: 1813 |
| AAV2 variant | 4096 | WO2018071831 SEQ ID NO: 1814 |
| AAV2/3 variant | 4097 | WO2018071831 SEQ ID NO: 1815 |
| AAV2/3 variant | 4098 | WO2018071831 SEQ ID NO: 1816 |
| AAV2/3 variant | 4099 | WO2018071831 SEQ ID NO: 1817 |
| AAV2/3 variant | 4100 | WO2018071831 SEQ ID NO: 1818 |
| AAV2/3 variant | 4101 | WO2018071831 SEQ ID NO: 1819 |
| AAV2/3 variant | 4102 | WO2018071831 SEQ ID NO: 1820 |
| AAV2/3 variant | 4103 | WO2018071831 SEQ ID NO: 1821 |
| AAV2/3 variant | 4104 | WO2018071831 SEQ ID NO: 1822 |
| AAV2/3 variant | 4105 | WO2018071831 SEQ ID NO: 1823 |
| AAV2/3 variant | 4106 | WO2018071831 SEQ ID NO: 1824 |
| AAV2/3 variant | 4107 | WO2018071831 SEQ ID NO: 1825 |
| AAV2/3 variant | 4108 | WO2018071831 SEQ ID NO: 1826 |
| AAV2/3 variant | 4109 | WO2018071831 SEQ ID NO: 1827 |
| AAV2/3 variant | 4110 | WO2018071831 SEQ ID NO: 1828 |
| AAV2/3 variant | 4111 | WO2018071831 SEQ ID NO: 1829 |
| AAV2/3 variant | 4112 | WO2018071831 SEQ ID NO: 1830 |
| AAV2/3 variant | 4113 | WO2018071831 SEQ ID NO: 1831 |
| AAV2/3 variant | 4114 | WO2018071831 SEQ ID NO: 1832 |
| AAV2/3 variant | 4115 | WO2018071831 SEQ ID NO: 1833 |
| AAV2/3 variant | 4116 | WO2018071831 SEQ ID NO: 1834 |
| AAV2/3 variant | 4117 | WO2018071831 SEQ ID NO: 1835 |
| AAV2/3 variant | 4118 | WO2018071831 SEQ ID NO: 1836 |
| AAV2/3 variant | 4119 | WO2018071831 SEQ ID NO: 1837 |
| AAV2/3 variant | 4120 | WO2018071831 SEQ ID NO: 1838 |
| AAV2/3 variant | 4121 | WO2018071831 SEQ ID NO: 1839 |
| AAV2/3 variant | 4122 | WO2018071831 SEQ ID NO: 1840 |
| AAV2/3 variant | 4123 | WO2018071831 SEQ ID NO: 1841 |
| AAV2/3 variant | 4124 | WO2018071831 SEQ ID NO: 1842 |
| AAV2/3 variant | 4125 | WO2018071831 SEQ ID NO: 1843 |
| AAV2/3 variant | 4126 | WO2018071831 SEQ ID NO: 1844 |
| AAV2/3 variant | 4127 | WO2018071831 SEQ ID NO: 1845 |
| AAV2/3 variant | 4128 | WO2018071831 SEQ ID NO: 1846 |
| AAV2/3 variant | 4129 | WO2018071831 SEQ ID NO: 1847 |
| AAV2/3 variant | 4130 | WO2018071831 SEQ ID NO: 1848 |
| AAV2/3 variant | 4131 | WO2018071831 SEQ ID NO: 1849 |
| AAV2/3 variant | 4132 | WO2018071831 SEQ ID NO: 1850 |
| AAV2/3 variant | 4133 | WO2018071831 SEQ ID NO: 1851 |
| AAV2/3 variant | 4134 | WO2018071831 SEQ ID NO: 1852 |
| AAV2/3 variant | 4135 | WO2018071831 SEQ ID NO: 1853 |
| AAV2/3 variant | 4136 | WO2018071831 SEQ ID NO: 1854 |
| AAV2/3 variant | 4137 | WO2018071831 SEQ ID NO: 1855 |
| AAV2/3 variant | 4138 | WO2018071831 SEQ ID NO: 1856 |
| AAV2/3 variant | 4139 | WO2018071831 SEQ ID NO: 1857 |
| AAV2/3 variant | 4140 | WO2018071831 SEQ ID NO: 1858 |
| AAV2/3 variant | 4141 | WO2018071831 SEQ ID NO: 1859 |
| AAV2/3 variant | 4142 | WO2018071831 SEQ ID NO: 1860 |
| AAV2/3 variant | 4143 | WO2018071831 SEQ ID NO: 1861 |
| AAV2/3 variant | 4144 | WO2018071831 SEQ ID NO: 1862 |
| AAV2/3 variant | 4145 | WO2018071831 SEQ ID NO: 1863 |
| AAV2/3 variant | 4146 | WO2018071831 SEQ ID NO: 1864 |
| AAV2/3 variant | 4147 | WO2018071831 SEQ ID NO: 1865 |
| AAV2/3 variant | 4148 | WO2018071831 SEQ ID NO: 1866 |
| AAV2/3 variant | 4149 | WO2018071831 SEQ ID NO: 1867 |
| AAV2/3 variant | 4150 | WO2018071831 SEQ ID NO: 1868 |
| AAV2/3 variant | 4151 | WO2018071831 SEQ ID NO: 1869 |
| AAV2/3 variant | 4152 | WO2018071831 SEQ ID NO: 1870 |
| AAV2/3 variant | 4153 | WO2018071831 SEQ ID NO: 1871 |
| AAV2/3 variant | 4154 | WO2018071831 SEQ ID NO: 1872 |
| AAV2/3 variant | 4155 | WO2018071831 SEQ ID NO: 1873 |
| AAV2/3 variant | 4156 | WO2018071831 SEQ ID NO: 1874 |
| AAV2/3 variant | 4157 | WO2018071831 SEQ ID NO: 1875 |
| AAV2/3 variant | 4158 | WO2018071831 SEQ ID NO: 1876 |
| AAV2/3 variant | 4159 | WO2018071831 SEQ ID NO: 1877 |
| AAV2/3 variant | 4160 | WO2018071831 SEQ ID NO: 1878 |
| AAV2/3 variant | 4161 | WO2018071831 SEQ ID NO: 1879 |
| AAV2/3 variant | 4162 | WO2018071831 SEQ ID NO: 1880 |
| AAV2/3 variant | 4163 | WO2018071831 SEQ ID NO: 1881 |
| AAV2/3 variant | 4164 | WO2018071831 SEQ ID NO: 1882 |
| AAV2/3 variant | 4165 | WO2018071831 SEQ ID NO: 1883 |
| AAV2/3 variant | 4166 | WO2018071831 SEQ ID NO: 1884 |
| AAV2/3 variant | 4167 | WO2018071831 SEQ ID NO: 1885 |
| AAV2/3 variant | 4168 | WO2018071831 SEQ ID NO: 1886 |
| AAV2/3 variant | 4169 | WO2018071831 SEQ ID NO: 1887 |
| AAV2/3 variant | 4170 | WO2018071831 SEQ ID NO: 1888 |
| AAV2/3 variant | 4171 | WO2018071831 SEQ ID NO: 1889 |
| AAV2/3 variant | 4172 | WO2018071831 SEQ ID NO: 1890 |
| AAV2/3 variant | 4173 | WO2018071831 SEQ ID NO: 1891 |
| AAV2/3 variant | 4174 | WO2018071831 SEQ ID NO: 1892 |
| AAV2/3 variant | 4175 | WO2018071831 SEQ ID NO: 1893 |
| AAV2/3 variant | 4176 | WO2018071831 SEQ ID NO: 1894 |
| AAV2/3 variant | 4177 | WO2018071831 SEQ ID NO: 1895 |
| AAV2/3 variant | 4178 | WO2018071831 SEQ ID NO: 1896 |
| AAV2/3 variant | 4179 | WO2018071831 SEQ ID NO: 1897 |
| AAV2/3 variant | 4180 | WO2018071831 SEQ ID NO: 1898 |
| AAV2/3 variant | 4181 | WO2018071831 SEQ ID NO: 1899 |
| AAV2/3 variant | 4182 | WO2018071831 SEQ ID NO: 1900 |
| AAV2/3 variant | 4183 | WO2018071831 SEQ ID NO: 1901 |
| AAV2/3 variant | 4184 | WO2018071831 SEQ ID NO: 1902 |
| AAV2/3 variant | 4185 | WO2018071831 SEQ ID NO: 1903 |
| AAV2/3 variant | 4186 | WO2018071831 SEQ ID NO: 1904 |
| AAV2/3 variant | 4187 | WO2018071831 SEQ ID NO: 1905 |
| AAV2/3 variant | 4188 | WO2018071831 SEQ ID NO: 1906 |
| AAV2/3 variant | 4189 | WO2018071831 SEQ ID NO: 1907 |
| AAV2/3 variant | 4190 | WO2018071831 SEQ ID NO: 1908 |
| AAV2/3 variant | 4191 | WO2018071831 SEQ ID NO: 1909 |
| AAV2/3 variant | 4192 | WO2018071831 SEQ ID NO: 1910 |
| AAV2/3 variant | 4193 | WO2018071831 SEQ ID NO: 1911 |
| AAV2/3 variant | 4194 | WO2018071831 SEQ ID NO: 1912 |
| AAV2/3 variant | 4195 | WO2018071831 SEQ ID NO: 1913 |
| AAV2/3 variant | 4196 | WO2018071831 SEQ ID NO: 1914 |
| AAV2/3 variant | 4197 | WO2018071831 SEQ ID NO: 1915 |
| AAV2/3 variant | 4198 | WO2018071831 SEQ ID NO: 1916 |
| AAV2/3 variant | 4199 | WO2018071831 SEQ ID NO: 1917 |
| AAV2/3 variant | 4200 | WO2018071831 SEQ ID NO: 1918 |
| AAV2/3 variant | 4201 | WO2018071831 SEQ ID NO: 1919 |
| AAV2/3 variant | 4202 | WO2018071831 SEQ ID NO: 1920 |
| AAV2/3 variant | 4203 | WO2018071831 SEQ ID NO: 1921 |
| AAV2/3 variant | 4204 | WO2018071831 SEQ ID NO: 1922 |
| AAV2/3 variant | 4205 | WO2018071831 SEQ ID NO: 1923 |
| AAV2/3 variant | 4206 | WO2018071831 SEQ ID NO: 1924 |
| AAV2/3 variant | 4207 | WO2018071831 SEQ ID NO: 1925 |
| AAV2/3 variant | 4208 | WO2018071831 SEQ ID NO: 1926 |
| AAV2/3 variant | 4209 | WO2018071831 SEQ ID NO: 1927 |
| AAV2/3 variant | 4210 | WO2018071831 SEQ ID NO: 1928 |
| AAV2/3 variant | 4211 | WO2018071831 SEQ ID NO: 1929 |
| AAV2/3 variant | 4212 | WO2018071831 SEQ ID NO: 1930 |
| AAV2/3 variant | 4213 | WO2018071831 SEQ ID NO: 1931 |
| AAV2/3 variant | 4214 | WO2018071831 SEQ ID NO: 1932 |
| AAV2/3 variant | 4215 | WO2018071831 SEQ ID NO: 1933 |
| AAV2/3 variant | 4216 | WO2018071831 SEQ ID NO: 1934 |
| AAV2/3 variant | 4217 | WO2018071831 SEQ ID NO: 1935 |
| AAV2/3 variant | 4218 | WO2018071831 SEQ ID NO: 1936 |
| AAV2/3 variant | 4219 | WO2018071831 SEQ ID NO: 1937 |
| AAV2/3 variant | 4220 | WO2018071831 SEQ ID NO: 1938 |
| AAV2/3 variant | 4221 | WO2018071831 SEQ ID NO: 1939 |
| AAV2/3 variant | 4222 | WO2018071831 SEQ ID NO: 1940 |
| AAV2/3 variant | 4223 | WO2018071831 SEQ ID NO: 1941 |
| AAV2/3 variant | 4224 | WO2018071831 SEQ ID NO: 1942 |
| AAV2/3 variant | 4225 | WO2018071831 SEQ ID NO: 1943 |
| AAV2/3 variant | 4226 | WO2018071831 SEQ ID NO: 1944 |
| AAV2/3 variant | 4227 | WO2018071831 SEQ ID NO: 1945 |
| AAV2/3 variant | 4228 | WO2018071831 SEQ ID NO: 1946 |
| AAV2/3 variant | 4229 | WO2018071831 SEQ ID NO: 1947 |
| AAV2/3 variant | 4230 | WO2018071831 SEQ ID NO: 1948 |
| AAV2/3 variant | 4231 | WO2018071831 SEQ ID NO: 1949 |
| AAV2/3 variant | 4232 | WO2018071831 SEQ ID NO: 1950 |
| AAV2/3 variant | 4233 | WO2018071831 SEQ ID NO: 1951 |
| AAV2/3 variant | 4234 | WO2018071831 SEQ ID NO: 1952 |
| AAV2/3 variant | 4235 | WO2018071831 SEQ ID NO: 1953 |
| AAV2/3 variant | 4236 | WO2018071831 SEQ ID NO: 1954 |
| AAV2/3 variant | 4237 | WO2018071831 SEQ ID NO: 1955 |
| AAV2/3 variant | 4238 | WO2018071831 SEQ ID NO: 1956 |
| AAV2/3 variant | 4239 | WO2018071831 SEQ ID NO: 1957 |
| AAV2/3 variant | 4240 | WO2018071831 SEQ ID NO: 1958 |
| AAV2/3 variant | 4241 | WO2018071831 SEQ ID NO: 1959 |
| AAV2/3 variant | 4242 | WO2018071831 SEQ ID NO: 1960 |
| AAV2/3 variant | 4243 | WO2018071831 SEQ ID NO: 1961 |
| AAV2/3 variant | 4244 | WO2018071831 SEQ ID NO: 1962 |
| AAV2/3 variant | 4245 | WO2018071831 SEQ ID NO: 1963 |
| AAV2/3 variant | 4246 | WO2018071831 SEQ ID NO: 1964 |
| AAV2/3 variant | 4247 | WO2018071831 SEQ ID NO: 1965 |
| AAV2/3 variant | 4248 | WO2018071831 SEQ ID NO: 1966 |
| AAV2/3 variant | 4249 | WO2018071831 SEQ ID NO: 1967 |
| AAV2/3 variant | 4250 | WO2018071831 SEQ ID NO: 1968 |
| AAV2/3 variant | 4251 | WO2018071831 SEQ ID NO: 1969 |
| AAV2/3 variant | 4252 | WO2018071831 SEQ ID NO: 1970 |
| AAV2/3 variant | 4253 | WO2018071831 SEQ ID NO: 1971 |
| AAV2/3 variant | 4254 | WO2018071831 SEQ ID NO: 1972 |
| AAV2/3 variant | 4255 | WO2018071831 SEQ ID NO: 1973 |
| AAV2/3 variant | 4256 | WO2018071831 SEQ ID NO: 1974 |
| AAV2/3 variant | 4257 | WO2018071831 SEQ ID NO: 1975 |
| AAV2/3 variant | 4258 | WO2018071831 SEQ ID NO: 1976 |
| AAV2/3 variant | 4259 | WO2018071831 SEQ ID NO: 1977 |
| AAV2/3 variant | 4260 | WO2018071831 SEQ ID NO: 1978 |
| AAV2/3 variant | 4261 | WO2018071831 SEQ ID NO: 1979 |
| AAV2/3 variant | 4262 | WO2018071831 SEQ ID NO: 1980 |
| AAV2/3 variant | 4263 | WO2018071831 SEQ ID NO: 1981 |
| AAV2/3 variant | 4264 | WO2018071831 SEQ ID NO: 1982 |
| AAV2/3 variant | 4265 | WO2018071831 SEQ ID NO: 1983 |
| AAV2/3 variant | 4266 | WO2018071831 SEQ ID NO: 1984 |
| AAV2/3 variant | 4267 | WO2018071831 SEQ ID NO: 1985 |
| AAV2/3 variant | 4268 | WO2018071831 SEQ ID NO: 1986 |
| AAV2/3 variant | 4269 | WO2018071831 SEQ ID NO: 1987 |
| AAV2/3 variant | 4270 | WO2018071831 SEQ ID NO: 1988 |
| AAV2 variant | 4271 | WO2018071831 SEQ ID NO: 1989 |
| AAV2 variant | 4272 | WO2018071831 SEQ ID NO: 1990 |
| AAV2 variant | 4273 | WO2018071831 SEQ ID NO: 1991 |
| AAV2 variant | 4274 | WO2018071831 SEQ ID NO: 1992 |
| AAV2 variant | 4275 | WO2018071831 SEQ ID NO: 1993 |
| AAV2 variant | 4276 | WO2018071831 SEQ ID NO: 1994 |
| AAV2 variant | 4277 | WO2018071831 SEQ ID NO: 1995 |
| AAV2 variant | 4278 | WO2018071831 SEQ ID NO: 1996 |
| AAV2 variant | 4279 | WO2018071831 SEQ ID NO: 1997 |
| AAV2 variant | 4280 | WO2018071831 SEQ ID NO: 1998 |
| AAV2 variant | 4281 | WO2018071831 SEQ ID NO: 1999 |
| AAV2 variant | 4282 | WO2018071831 SEQ ID NO: 2000 |
| AAV2 variant | 4283 | WO2018071831 SEQ ID NO: 2001 |
| AAV2 variant | 4284 | WO2018071831 SEQ ID NO: 2002 |
| AAV2 variant | 4285 | WO2018071831 SEQ ID NO: 2003 |
| AAV2 variant | 4286 | WO2018071831 SEQ ID NO: 2004 |
| AAV2 variant | 4287 | WO2018071831 SEQ ID NO: 2005 |
| AAV2 variant | 4288 | WO2018071831 SEQ ID NO: 2006 |
| AAV2 variant | 4289 | WO2018071831 SEQ ID NO: 2007 |
| AAV2 variant | 4290 | WO2018071831 SEQ ID NO: 2008 |
| AAV2 variant | 4291 | WO2018071831 SEQ ID NO: 2009 |
| AAV2 variant | 4292 | WO2018071831 SEQ ID NO: 2010 |
| AAV2 variant | 4293 | WO2018071831 SEQ ID NO: 2011 |
| AAV2 variant | 4294 | WO2018071831 SEQ ID NO: 2012 |
| AAV2 variant | 4295 | WO2018071831 SEQ ID NO: 2013 |
| AAV2 variant | 4296 | WO2018071831 SEQ ID NO: 2014 |
| AAV2 variant | 4297 | WO2018071831 SEQ ID NO: 2015 |
| AAV2 variant | 4298 | WO2018071831 SEQ ID NO: 2016 |
| AAV2 variant | 4299 | WO2018071831 SEQ ID NO: 2017 |
| AAV2 variant | 4300 | WO2018071831 SEQ ID NO: 2018 |
| AAV2 variant | 4301 | WO2018071831 SEQ ID NO: 2019 |
| AAV2 variant | 4302 | WO2018071831 SEQ ID NO: 2020 |
| AAV2 variant | 4303 | WO2018071831 SEQ ID NO: 2021 |
| AAV2 variant | 4304 | WO2018071831 SEQ ID NO: 2022 |
| AAV2 variant | 4305 | WO2018071831 SEQ ID NO: 2023 |
| AAV2 variant | 4306 | WO2018071831 SEQ ID NO: 2024 |
| AAV2 variant | 4307 | WO2018071831 SEQ ID NO: 2025 |
| AAV2 variant | 4308 | WO2018071831 SEQ ID NO: 2026 |
| AAV2 variant | 4309 | WO2018071831 SEQ ID NO: 2027 |
| AAV2 variant | 4310 | WO2018071831 SEQ ID NO: 2028 |
| AAV2 variant | 4311 | WO2018071831 SEQ ID NO: 2029 |
| AAV2 variant | 4312 | WO2018071831 SEQ ID NO: 2030 |
| AAV2 variant | 4313 | WO2018071831 SEQ ID NO: 2031 |
| AAV2 variant | 4314 | WO2018071831 SEQ ID NO: 2032 |
| AAV2 variant | 4315 | WO2018071831 SEQ ID NO: 2033 |
| AAV2 variant | 4316 | WO2018071831 SEQ ID NO: 2034 |
| AAV2 variant | 4317 | WO2018071831 SEQ ID NO: 2035 |
| AAV2 variant | 4318 | WO2018071831 SEQ ID NO: 2036 |
| AAV2 variant | 4319 | WO2018071831 SEQ ID NO: 2037 |
| AAV2 variant | 4320 | WO2018071831 SEQ ID NO: 2038 |
| AAV2 variant | 4321 | WO2018071831 SEQ ID NO: 2039 |
| AAV2 variant | 4322 | WO2018071831 SEQ ID NO: 2040 |
| AAV2 variant | 4323 | WO2018071831 SEQ ID NO: 2041 |
| AAV2 variant | 4324 | WO2018071831 SEQ ID NO: 2042 |
| AAV2 variant | 4325 | WO2018071831 SEQ ID NO: 2043 |
| AAV2 variant | 4326 | WO2018071831 SEQ ID NO: 2044 |
| AAV2 variant | 4327 | WO2018071831 SEQ ID NO: 2045 |
| AAV2 variant | 4328 | WO2018071831 SEQ ID NO: 2046 |
| AAV2 variant | 4329 | WO2018071831 SEQ ID NO: 2047 |
| AAV2 variant | 4330 | WO2018071831 SEQ ID NO: 2048 |
| AAV2 variant | 4331 | WO2018071831 SEQ ID NO: 2049 |
| AAV2 variant | 4332 | WO2018071831 SEQ ID NO: 2050 |
| AAV2 variant | 4333 | WO2018071831 SEQ ID NO: 2051 |
| AAV2 variant | 4334 | WO2018071831 SEQ ID NO: 2052 |
| AAV2 variant | 4335 | WO2018071831 SEQ ID NO: 2053 |
| AAV2 variant | 4336 | WO2018071831 SEQ ID NO: 2054 |
| AAV2 variant | 4337 | WO2018071831 SEQ ID NO: 2055 |
| AAV2 variant | 4338 | WO2018071831 SEQ ID NO: 2056 |
| AAV2 variant | 4339 | WO2018071831 SEQ ID NO: 2057 |
| AAV2 variant | 4340 | WO2018071831 SEQ ID NO: 2058 |
| AAV2 variant | 4341 | WO2018071831 SEQ ID NO: 2059 |
| AAV2 variant | 4342 | WO2018071831 SEQ ID NO: 2060 |
| AAV2 variant | 4343 | WO2018071831 SEQ ID NO: 2061 |
| AAV2 variant | 4344 | WO2018071831 SEQ ID NO: 2062 |
| AAV2 variant | 4345 | WO2018071831 SEQ ID NO: 2063 |
| AAV2 variant | 4346 | WO2018071831 SEQ ID NO: 2064 |
| AAV2 variant | 4347 | WO2018071831 SEQ ID NO: 2065 |
| AAV2 variant | 4348 | WO2018071831 SEQ ID NO: 2066 |
| AAV2 variant | 4349 | WO2018071831 SEQ ID NO: 2067 |
| AAV2 variant | 4350 | WO2018071831 SEQ ID NO: 2068 |
| AAV2 variant | 4351 | WO2018071831 SEQ ID NO: 2069 |
| AAV2 variant | 4352 | WO2018071831 SEQ ID NO: 2070 |
| AAV2 variant | 4353 | WO2018071831 SEQ ID NO: 2071 |
| AAV2 variant | 4354 | WO2018071831 SEQ ID NO: 2072 |
| AAV2 variant | 4355 | WO2018071831 SEQ ID NO: 2073 |
| AAV2 variant | 4356 | WO2018071831 SEQ ID NO: 2074 |
| AAV2 variant | 4357 | WO2018071831 SEQ ID NO: 2075 |
| AAV2 variant | 4358 | WO2018071831 SEQ ID NO: 2076 |
| AAV2 variant | 4359 | WO2018071831 SEQ ID NO: 2077 |
| AAV2/3 variant | 4360 | WO2018071831 SEQ ID NO: 2078 |
| AAV2/3 variant | 4361 | WO2018071831 SEQ ID NO: 2079 |
| AAV2/3 variant | 4362 | WO2018071831 SEQ ID NO: 2080 |
| AAV2/3 variant | 4363 | WO2018071831 SEQ ID NO: 2081 |
| AAV2/3 variant | 4364 | WO2018071831 SEQ ID NO: 2082 |
| AAV2/3 variant | 4365 | WO2018071831 SEQ ID NO: 2083 |
| AAV2/3 variant | 4366 | WO2018071831 SEQ ID NO: 2084 |
| AAV2/3 variant | 4367 | WO2018071831 SEQ ID NO: 2085 |
| AAV2/3 variant | 4368 | WO2018071831 SEQ ID NO: 2086 |
| AAV2/3 variant | 4369 | WO2018071831 SEQ ID NO: 2087 |
| AAV2/3 variant | 4370 | WO2018071831 SEQ ID NO: 2088 |
| AAV2/3 variant | 4371 | WO2018071831 SEQ ID NO: 2089 |
| AAV2/3 variant | 4372 | WO2018071831 SEQ ID NO: 2090 |
| AAV2/3 variant | 4373 | WO2018071831 SEQ ID NO: 2091 |
| AAV2/3 variant | 4374 | WO2018071831 SEQ ID NO: 2092 |
| AAV2/3 variant | 4375 | WO2018071831 SEQ ID NO: 2093 |
| AAV2/3 variant | 4376 | WO2018071831 SEQ ID NO: 2094 |
| AAV2/3 variant | 4377 | WO2018071831 SEQ ID NO: 2095 |
| AAV2/3 variant | 4378 | WO2018071831 SEQ ID NO: 2096 |
| AAV2/3 variant | 4379 | WO2018071831 SEQ ID NO: 2097 |
| AAV2/3 variant | 4380 | WO2018071831 SEQ ID NO: 2098 |
| AAV2/3 variant | 4381 | WO2018071831 SEQ ID NO: 2099 |
| AAV2/3 variant | 4382 | WO2018071831 SEQ ID NO: 2100 |
| AAV2/3 variant | 4383 | WO2018071831 SEQ ID NO: 2101 |
| AAV2/3 variant | 4384 | WO2018071831 SEQ ID NO: 2102 |
| AAV2/3 variant | 4385 | WO2018071831 SEQ ID NO: 2103 |
| AAV2/3 variant | 4386 | WO2018071831 SEQ ID NO: 2104 |
| AAV2/3 variant | 4387 | WO2018071831 SEQ ID NO: 2105 |
| AAV2/3 variant | 4388 | WO2018071831 SEQ ID NO: 2106 |
| AAV2/3 variant | 4389 | WO2018071831 SEQ ID NO: 2107 |
| AAV2/3 variant | 4390 | WO2018071831 SEQ ID NO: 2108 |
| AAV2/3 variant | 4391 | WO2018071831 SEQ ID NO: 2109 |
| AAV2/3 variant | 4392 | WO2018071831 SEQ ID NO: 2110 |
| AAV2/3 variant | 4393 | WO2018071831 SEQ ID NO: 2111 |
| AAV2/3 variant | 4394 | WO2018071831 SEQ ID NO: 2112 |
| AAV2/3 variant | 4395 | WO2018071831 SEQ ID NO: 2113 |
| AAV2/3 variant | 4396 | WO2018071831 SEQ ID NO: 2114 |
| AAV2/3 variant | 4397 | WO2018071831 SEQ ID NO: 2115 |
| AAV2/3 variant | 4398 | WO2018071831 SEQ ID NO: 2116 |
| AAV2/3 variant | 4399 | WO2018071831 SEQ ID NO: 2117 |
| AAV2/3 variant | 4400 | WO2018071831 SEQ ID NO: 2118 |
| AAV2/3 variant | 4401 | WO2018071831 SEQ ID NO: 2119 |
| AAV2/3 variant | 4402 | WO2018071831 SEQ ID NO: 2120 |
| AAV2/3 variant | 4403 | WO2018071831 SEQ ID NO: 2121 |
| AAV2/3 variant | 4404 | WO2018071831 SEQ ID NO: 2122 |
| AAV2/3 variant | 4405 | WO2018071831 SEQ ID NO: 2123 |
| AAV2/3 variant | 4406 | WO2018071831 SEQ ID NO: 2124 |
| AAV2/3 variant | 4407 | WO2018071831 SEQ ID NO: 2125 |
| AAV2/3 variant | 4408 | WO2018071831 SEQ ID NO: 2126 |
| AAV2/3 variant | 4409 | WO2018071831 SEQ ID NO: 2127 |
| AAV2/3 variant | 4410 | WO2018071831 SEQ ID NO: 2128 |
| AAV2/3 variant | 4411 | WO2018071831 SEQ ID NO: 2129 |
| AAV2/3 variant | 4412 | WO2018071831 SEQ ID NO: 2130 |
| AAV2/3 variant | 4413 | WO2018071831 SEQ ID NO: 2131 |
| AAV2/3 variant | 4414 | WO2018071831 SEQ ID NO: 2132 |
| AAV2/3 variant | 4415 | WO2018071831 SEQ ID NO: 2133 |
| AAV2/3 variant | 4416 | WO2018071831 SEQ ID NO: 2134 |
| AAV2/3 variant | 4417 | WO2018071831 SEQ ID NO: 2135 |
| AAV2/3 variant | 4418 | WO2018071831 SEQ ID NO: 2136 |
| AAV2/3 variant | 4419 | WO2018071831 SEQ ID NO: 2137 |
| AAV2/3 variant | 4420 | WO2018071831 SEQ ID NO: 2138 |
| AAV2/3 variant | 4421 | WO2018071831 SEQ ID NO: 2139 |
| AAV2/3 variant | 4422 | WO2018071831 SEQ ID NO: 2140 |
| AAV2/3 variant | 4423 | WO2018071831 SEQ ID NO: 2141 |
| AAV2/3 variant | 4424 | WO2018071831 SEQ ID NO: 2142 |
| AAV2/3 variant | 4425 | WO2018071831 SEQ ID NO: 2143 |
| AAV2/3 variant | 4426 | WO2018071831 SEQ ID NO: 2144 |
| AAV2/3 variant | 4427 | WO2018071831 SEQ ID NO: 2145 |
| AAV2/3 variant | 4428 | WO2018071831 SEQ ID NO: 2146 |
| AAV2/3 variant | 4429 | WO2018071831 SEQ ID NO: 2147 |
| AAV2/3 variant | 4430 | WO2018071831 SEQ ID NO: 2148 |
| AAV2/3 variant | 4431 | WO2018071831 SEQ ID NO: 2149 |
| AAV2/3 variant | 4432 | WO2018071831 SEQ ID NO: 2150 |
| AAV2/3 variant | 4433 | WO2018071831 SEQ ID NO: 2151 |
| AAV2/3 variant | 4434 | WO2018071831 SEQ ID NO: 2152 |
| AAV2/3 variant | 4435 | WO2018071831 SEQ ID NO: 2153 |
| AAV2/3 variant | 4436 | WO2018071831 SEQ ID NO: 2154 |
| AAV2/3 variant | 4437 | WO2018071831 SEQ ID NO: 2155 |
| AAV2/3 variant | 4438 | WO2018071831 SEQ ID NO: 2156 |
| AAV2/3 variant | 4439 | WO2018071831 SEQ ID NO: 2157 |
| AAV2/3 variant | 4440 | WO2018071831 SEQ ID NO: 2158 |
| AAV2/3 variant | 4441 | WO2018071831 SEQ ID NO: 2159 |
| AAV2/3 variant | 4442 | WO2018071831 SEQ ID NO: 2160 |
| AAV2/3 variant | 4443 | WO2018071831 SEQ ID NO: 2161 |
| AAV2/3 variant | 4444 | WO2018071831 SEQ ID NO: 2162 |
| AAV2/3 variant | 4445 | WO2018071831 SEQ ID NO: 2163 |
| AAV2/3 variant | 4446 | WO2018071831 SEQ ID NO: 2164 |
| AAV2/3 variant | 4447 | WO2018071831 SEQ ID NO: 2165 |
| AAV2/3 variant | 4448 | WO2018071831 SEQ ID NO: 2166 |
| AAV2/3 variant | 4449 | WO2018071831 SEQ ID NO: 2167 |
| AAV2/3 variant | 4450 | WO2018071831 SEQ ID NO: 2168 |
| AAV2/3 variant | 4451 | WO2018071831 SEQ ID NO: 2169 |
| AAV2/3 variant | 4452 | WO2018071831 SEQ ID NO: 2170 |
| AAV2/3 variant | 4453 | WO2018071831 SEQ ID NO: 2171 |
| AAV2/3 variant | 4454 | WO2018071831 SEQ ID NO: 2172 |
| AAV2/3 variant | 4455 | WO2018071831 SEQ ID NO: 2173 |
| AAV2/3 variant | 4456 | WO2018071831 SEQ ID NO: 2174 |
| AAV2/3 variant | 4457 | WO2018071831 SEQ ID NO: 2175 |
| AAV2/3 variant | 4458 | WO2018071831 SEQ ID NO: 2176 |
| AAV2/3 variant | 4459 | WO2018071831 SEQ ID NO: 2177 |
| AAV2/3 variant | 4460 | WO2018071831 SEQ ID NO: 2178 |
| AAV2/3 variant | 4461 | WO2018071831 SEQ ID NO: 2179 |
| AAV2/3 variant | 4462 | WO2018071831 SEQ ID NO: 2180 |
| AAV2/3 variant | 4463 | WO2018071831 SEQ ID NO: 2181 |
| AAV2/3 variant | 4464 | WO2018071831 SEQ ID NO: 2182 |
| AAV2/3 variant | 4465 | WO2018071831 SEQ ID NO: 2183 |
| AAV2/3 variant | 4466 | WO2018071831 SEQ ID NO: 2184 |
| AAV2/3 variant | 4467 | WO2018071831 SEQ ID NO: 2185 |
| AAV2/3 variant | 4468 | WO2018071831 SEQ ID NO: 2186 |
| AAV2/3 variant | 4469 | WO2018071831 SEQ ID NO: 2187 |
| AAV2/3 variant | 4470 | WO2018071831 SEQ ID NO: 2188 |
| AAV2/3 variant | 4471 | WO2018071831 SEQ ID NO: 2189 |
| AAV2/3 variant | 4472 | WO2018071831 SEQ ID NO: 2190 |
| AAV2/3 variant | 4473 | WO2018071831 SEQ ID NO: 2191 |
| AAV2/3 variant | 4474 | WO2018071831 SEQ ID NO: 2192 |
| AAV2/3 variant | 4475 | WO2018071831 SEQ ID NO: 2193 |
| AAV2/3 variant | 4476 | WO2018071831 SEQ ID NO: 2194 |
| AAV2/3 variant | 4477 | WO2018071831 SEQ ID NO: 2195 |
| AAV2/3 variant | 4478 | WO2018071831 SEQ ID NO: 2196 |
| AAV2/3 variant | 4479 | WO2018071831 SEQ ID NO: 2197 |
| AAV2/3 variant | 4480 | WO2018071831 SEQ ID NO: 2198 |
| AAV2/3 variant | 4481 | WO2018071831 SEQ ID NO: 2199 |
| AAV2/3 variant | 4482 | WO2018071831 SEQ ID NO: 2200 |
| AAV2/3 variant | 4483 | WO2018071831 SEQ ID NO: 2201 |
| AAV2/3 variant | 4484 | WO2018071831 SEQ ID NO: 2202 |
| AAV2/3 variant | 4485 | WO2018071831 SEQ ID NO: 2203 |
| AAV2/3 variant | 4486 | WO2018071831 SEQ ID NO: 2204 |
| AAV2/3 variant | 4487 | WO2018071831 SEQ ID NO: 2205 |
| AAV2/3 variant | 4488 | WO2018071831 SEQ ID NO: 2206 |
| AAV2/3 variant | 4489 | WO2018071831 SEQ ID NO: 2207 |
| AAV2/3 variant | 4490 | WO2018071831 SEQ ID NO: 2208 |
| AAV2/3 variant | 4491 | WO2018071831 SEQ ID NO: 2209 |
| AAV2/3 variant | 4492 | WO2018071831 SEQ ID NO: 2210 |
| AAV2/3 variant | 4493 | WO2018071831 SEQ ID NO: 2211 |
| AAV2/3 variant | 4494 | WO2018071831 SEQ ID NO: 2212 |
| AAV2/3 variant | 4495 | WO2018071831 SEQ ID NO: 2213 |
| AAV2/3 variant | 4496 | WO2018071831 SEQ ID NO: 2214 |
| AAV2/3 variant | 4497 | WO2018071831 SEQ ID NO: 2215 |
| AAV2/3 variant | 4498 | WO2018071831 SEQ ID NO: 2216 |
| AAV2/3 variant | 4499 | WO2018071831 SEQ ID NO: 2217 |
| AAV2/3 variant | 4500 | WO2018071831 SEQ ID NO: 2218 |
| AAV2/3 variant | 4501 | WO2018071831 SEQ ID NO: 2219 |
| AAV2/3 variant | 4502 | WO2018071831 SEQ ID NO: 2220 |
| AAV2/3 variant | 4503 | WO2018071831 SEQ ID NO: 2221 |
| AAV2/3 variant | 4504 | WO2018071831 SEQ ID NO: 2222 |
| AAV2/3 variant | 4505 | WO2018071831 SEQ ID NO: 2223 |
| AAV2/3 variant | 4506 | WO2018071831 SEQ ID NO: 2224 |
| AAV2/3 variant | 4507 | WO2018071831 SEQ ID NO: 2225 |
| AAV2/3 variant | 4508 | WO2018071831 SEQ ID NO: 2226 |
| AAV2/3 variant | 4509 | WO2018071831 SEQ ID NO: 2227 |
| AAV2/3 variant | 4510 | WO2018071831 SEQ ID NO: 2228 |
| AAV2/3 variant | 4511 | WO2018071831 SEQ ID NO: 2229 |
| AAV2/3 variant | 4512 | WO2018071831 SEQ ID NO: 2230 |
| AAV2/3 variant | 4513 | WO2018071831 SEQ ID NO: 2231 |
| AAV2/3 variant | 4514 | WO2018071831 SEQ ID NO: 2232 |
| AAV2/3 variant | 4515 | WO2018071831 SEQ ID NO: 2233 |
| AAV2/3 variant | 4516 | WO2018071831 SEQ ID NO: 2234 |
| AAV2/3 variant | 4517 | WO2018071831 SEQ ID NO: 2235 |
| AAV2/3 variant | 4518 | WO2018071831 SEQ ID NO: 2236 |
| AAV2/3 variant | 4519 | WO2018071831 SEQ ID NO: 2237 |
| AAV2/3 variant | 4520 | WO2018071831 SEQ ID NO: 2238 |
| AAV2/3 variant | 4521 | WO2018071831 SEQ ID NO: 2239 |
| AAV2/3 variant | 4522 | WO2018071831 SEQ ID NO: 2240 |
| AAV2/3 variant | 4523 | WO2018071831 SEQ ID NO: 2241 |
| AAV2/3 variant | 4524 | WO2018071831 SEQ ID NO: 2242 |
| AAV2/3 variant | 4525 | WO2018071831 SEQ ID NO: 2243 |
| AAV2/3 variant | 4526 | WO2018071831 SEQ ID NO: 2244 |
| AAV2/3 variant | 4527 | WO2018071831 SEQ ID NO: 2245 |
| AAV2/3 variant | 4528 | WO2018071831 SEQ ID NO: 2246 |
| AAV2/3 variant | 4529 | WO2018071831 SEQ ID NO: 2247 |
| AAV2/3 variant | 4530 | WO2018071831 SEQ ID NO: 2248 |
| AAV2/3 variant | 4531 | WO2018071831 SEQ ID NO: 2249 |
| AAV2/3 variant | 4532 | WO2018071831 SEQ ID NO: 2250 |
| AAV2/3 variant | 4533 | WO2018071831 SEQ ID NO: 2251 |

In some embodiments, the AAV serotype may be, or may have a sequence as described in International Patent Publication WO2015038958, the contents of which are herein incorporated by reference in their entirety, such as, but not limited to, AAV9 (SEQ ID NO: 2 and 11 of WO2015038958 or SEQ ID NO: 137 and 138 respectively herein), PHP.B (SEQ ID NO: 8 and 9 of WO2015038958, herein SEQ ID NO: 5 and 6), G2B-13 (SEQ ID NO: 12 of WO2015038958, herein SEQ ID NO: 7), G2B-26 (SEQ ID NO: 13 of WO2015038958, herein SEQ ID NO: 5), TH1.1-32 (SEQ ID NO: 14 of WO2015038958, herein SEQ ID NO: 8), TH1.1-35 (SEQ ID NO: 15 of WO2015038958, herein SEQ ID NO: 9) or variants thereof. Further, any of the targeting peptides or amino acid inserts described in WO2015038958, may be inserted into any parent AAV serotype, such as, but not limited to, AAV9 (SEQ ID NO: 137 for the DNA sequence and SEQ ID NO: 138 for the amino acid sequence). In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, TLAVPFK (SEQ ID NO: 1 of WO2015038958; herein SEQ ID NO: 1262), KFPVALT (SEQ ID NO: 3 of WO2015038958; herein SEQ ID NO: 1263), LAVPFK (SEQ ID NO: 31 of WO2015038958; herein SEQ ID NO: 1264), AVPFK (SEQ ID NO: 32 of WO2015038958; herein SEQ ID NO: 1265), VPFK (SEQ ID NO: 33 of WO2015038958; herein SEQ ID NO: 1266), TLAVPF (SEQ ID NO: 34 of WO2015038958; herein SEQ ID NO: 1267), TLAVP (SEQ ID NO: 35 of WO2015038958; herein SEQ ID NO: 1268), TLAV (SEQ ID NO: 36 of WO2015038958; herein SEQ ID NO: 1269), SVSKPFL (SEQ ID NO: 28 of WO2015038958; herein SEQ ID NO: 1270), FTLTTPK (SEQ ID NO: 29 of WO2015038958; herein SEQ ID NO: 1271), MNATKNV (SEQ ID NO: 30 of WO2015038958; herein SEQ ID NO: 1272), QSSQTPR (SEQ ID NO: 54 of WO2015038958; herein SEQ ID NO: 1273), ILGTGTS (SEQ ID NO: 55 of WO2015038958; herein SEQ ID NO: 1274), TRTNPEA (SEQ ID NO: 56 of WO2015038958; herein SEQ ID NO: 1275), NGGTSSS (SEQ ID NO: 58 of WO2015038958; herein SEQ ID NO: 1276), or YTLSQGW (SEQ ID NO: 60 of WO2015038958; herein SEQ ID NO: 1277). Non-limiting examples of nucleotide sequences that may encode the amino acid inserts include the following, AAGTTTCCTGTGGCGTTGACT (for SEQ ID NO: 3 of WO2015038958; herein SEQ ID NO: 1278), ACTTTGGCGGTGCCTTTTAAG (SEQ ID NO: 24 and 49 of WO2015038958; herein SEQ ID NO: 1279), AGTGTGAGTAAGCCTTTTTTG (SEQ ID NO: 25 of WO2015038958; herein SEQ ID NO: 1280), TTTACGTTGACGACGCCTAAG (SEQ ID NO: 26 of WO2015038958; herein SEQ ID NO: 1281), ATGAATGCTACGAAGAATGTG (SEQ ID NO: 27 of WO2015038958; herein SEQ ID NO: 1282), CAGTCGTCGCAGACGCCTAGG (SEQ ID NO: 48 of WO2015038958; herein SEQ ID NO: 1283), ATTCTGGGGACTGGTACTTCG (SEQ ID NO: 50 and 52 of WO2015038958; herein SEQ ID NO: 1284). ACGCGGACTAATCCTGAGGCT (SEQ ID NO: 51 of WO2015038958; herein SEQ ID NO: 1285). AATGGGGGGACTAGTAGTTCT (SEQ ID NO: 53 of WO2015038958; herein SEQ ID NO: 1286), or TATACTTTGTCGCAGGGTTGG (SEQ ID NO: 59 of WO2015038958; herein SEQ ID NO: 1287).

In some embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 11). PHP.N (SEQ ID NO: 46 of WO2017100671, herein SEQ ID NO: 4), PHP.S (SEQ ID NO: 47 of WO2017100671, herein SEQ ID NO: 10), or variants thereof. Further, any of the targeting peptides or amino acid inserts described in WO2017100671 may be inserted into any parent AAV serotype, such as, but not limited to, AAV9. In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, AQTLAVPFKAQ (SEQ ID NO: 1 of WO2017100671; herein SEQ ID NO: 1288), AQSVSKPFLAQ (SEQ ID NO: 2 of WO2017100671; herein SEQ ID NO: 1289), AQFTLTTPKAQ (SEQ ID NO: 3 in the sequence listing of WO2017100671; herein SEQ ID NO: 1290), DGTLAVPFKAQ (SEQ ID NO: 4 in the sequence listing of WO2017100671; herein SEQ ID NO: 1291), ESTLAVPFKAQ (SEQ ID NO: 5 of WO2017100671; herein SEQ ID NO: 1292), GGTLAVPFKAQ (SEQ ID NO: 6 of WO2017100671; herein SEQ ID NO: 1293), AQTLATPFKAQ (SEQ ID NO: 7 and 33 of WO2017100671; herein SEQ ID NO: 1294), ATTLATPFKAQ (SEQ ID NO: 8 of WO2017100671; herein SEQ ID NO: 1295), DGTLATPFKAQ (SEQ ID NO: 9 of WO2017100671; herein SEQ ID NO: 1296), GGTLATPFKAQ (SEQ ID NO: 10 of WO2017100671; herein SEQ ID NO: 1297), SGSLAVPFKAQ (SEQ ID NO: 11 of WO2017100671; herein SEQ ID NO: 1298), AQTLAQPFKAQ (SEQ ID NO: 12 of WO2017100671; herein SEQ ID NO: 1299), AQTLQQPFKAQ (SEQ ID NO: 13 of WO2017100671; herein SEQ ID NO: 1300), AQTLSNPFKAQ (SEQ ID NO: 14 of WO2017100671; herein SEQ ID NO: 1301), AQTLAVPFSNP (SEQ ID NO: 15 of WO2017100671; herein SEQ ID NO: 1302), QGTLAVPFKAQ (SEQ ID NO: 16 of WO2017100671; herein SEQ ID NO: 1303), NQTLAVPFKAQ (SEQ ID NO: 17 of WO2017100671; herein SEQ ID NO: 1304), EGSLAVPFKAQ (SEQ ID NO: 18 of WO2017100671; herein SEQ ID NO: 1305), SGNLAVPFKAQ (SEQ ID NO: 19 of WO2017100671; herein SEQ ID NO: 1306), EGTLAVPFKAQ (SEQ ID NO: 20 of WO2017100671; herein SEQ ID NO: 1307), DSTLAVPFKAQ (SEQ ID NO: 21 in Table 1 of WO2017100671; herein SEQ ID NO: 1308), AVTLAVPFKAQ (SEQ ID NO: 22 of WO2017100671; herein SEQ ID NO: 1309), AQTLSTPFKAQ (SEQ ID NO: 23 of WO2017100671; herein SEQ ID NO: 1310), AQTLPQPFKAQ (SEQ ID NO: 24 and 32 of WO2017100671; herein SEQ ID NO: 1311), AQTLSQPFKAQ (SEQ ID NO: 25 of WO2017100671; herein SEQ ID NO: 1312), AQTLQLPFKAQ (SEQ ID NO: 26 of WO2017100671; herein SEQ ID NO: 1313), AQTLTMPFKAQ (SEQ ID NO: 27, and 34 of WO2017100671 and SEQ ID NO: 35 in the sequence listing of WO2017100671; herein SEQ ID NO: 1314), AQTLTTPFKAQ (SEQ ID NO: 28 of WO2017100671; herein SEQ ID NO: 1315), AQYTLSQGWAQ (SEQ ID NO: 29 of WO2017100671; herein SEQ ID NO: 1316), AQMNATKNVAQ (SEQ ID NO: 30 of WO2017100671; herein SEQ ID NO: 1317), AQVSGGHHSAQ (SEQ ID NO: 31 of WO2017100671; herein SEQ ID NO: 1318), AQTLTAPFKAQ (SEQ ID NO: 35 in Table 1 of WO2017100671; herein SEQ ID NO: 1319), AQTLSKPFKAQ (SEQ ID NO: 36 of WO2017100671; herein SEQ ID NO: 1320), QAVRTSL (SEQ ID NO: 37 of WO2017100671; herein SEQ ID NO: 1321), YTLSQGW (SEQ ID NO: 38 of WO2017100671; herein SEQ ID NO: 1277), LAKERLS (SEQ ID NO: 39 of WO2017100671; herein SEQ ID NO: 1322), TLAVPFK (SEQ ID NO: 40 in the sequence listing of WO2017100671; herein SEQ ID NO: 1262), SVSKPFL (SEQ ID NO: 41 of WO2017100671; herein SEQ ID NO: 1270), FTLTTPK (SEQ ID NO: 42 of WO2017100671; herein SEQ ID NO: 1271), MNSTKNV (SEQ ID NO: 43 of WO2017100671; herein SEQ ID NO: 1323), VSGGHHS (SEQ ID NO: 44 of WO2017100671, herein SEQ ID NO: 1324), SAQTLAVPFKAQAQ (SEQ ID NO: 48 of WO2017100671; herein SEQ ID NO: 1325), SXXXLAVPFKAQAQ (SEQ ID NO: 49 of WO2017100671 wherein X may be any amino acid; herein SEQ ID NO: 1326), SAQXXXVPFKAQAQ (SEQ ID NO: 50 of WO2017100671 wherein X may be any amino acid; herein SEQ ID NO: 1327), SAQTLXXXFKAQAQ (SEQ ID NO: 51 of WO2017100671 wherein X may be any amino acid; herein SEQ ID NO: 1328), SAQTLAVXXXAQAQ (SEQ ID NO: 52 of WO2017100671 wherein X may be any amino acid; herein SEQ ID NO: 1329). SAQTLAVPFXXXAQ (SEQ ID NO: 53 of WO2017100671 wherein X may be any amino acid; herein SEQ ID NO: 1330), TNHQSAQ (SEQ ID NO: 65 of WO2017100671; herein SEQ ID NO: 1331), AQAQTGW (SEQ ID NO: 66 of WO2017100671; herein SEQ ID NO: 1332), DGTLATPFK (SEQ ID NO: 67 of WO2017100671; herein SEQ ID NO: 1333), DGTLATPFKXX (SEQ ID NO: 68 of WO2017100671 wherein X may be any amino acid; herein SEQ ID NO: 1334), LAVPFKAQ (SEQ ID NO: 80 of WO2017100671; herein SEQ ID NO: 1335), VPFKAQ (SEQ ID NO: 81 of WO2017100671; herein SEQ ID NO: 1336), FKAQ (SEQ ID NO: 82 of WO2017100671; herein SEQ ID NO: 1337), AQTLAV (SEQ ID NO: 83 of WO2017100671; herein SEQ ID NO: 1338), AQTLAVPF (SEQ ID NO: 84 of WO2017100671; herein SEQ ID NO: 1339). QAVR (SEQ ID NO: 85 of WO2017100671; herein SEQ ID NO: 1340), AVRT (SEQ ID NO: 86 of WO2017100671; herein SEQ ID NO: 1341), VRTS (SEQ ID NO: 87 of WO2017100671; herein SEQ ID NO: 1342), RTSL (SEQ ID NO: 88 of WO2017100671; herein SEQ ID NO: 1343), QAVRT (SEQ ID NO: 89 of WO2017100671; herein SEQ ID NO: 1344), AVRTS (SEQ ID NO: 90 of WO2017100671; herein SEQ ID NO: 1345). VRTSL (SEQ ID NO: 91 of WO2017100671; herein SEQ ID NO: 1346), QAVRTS (SEQ ID NO: 92 of WO2017100671; herein SEQ ID NO: 1347), or AVRTSL (SEQ ID NO: 93 of WO2017100671; herein SEQ ID NO: 1348).

Non-limiting examples of nucleotide sequences that may encode the amino acid inserts include the following, GATGGGACTTTGGCGGTGCCTITTAAGGCACAG (SEQ ID NO: 54 of WO2017100671; herein SEQ ID NO: 1349), GATGGGACGTTGGCGGTGCCTITTAAGGCACAG (SEQ ID NO: 55 of WO2017100671; herein SEQ ID NO: 1350), CAGGCGGTTAGGACGTCTTTG (SEQ ID NO: 56 of WO2017100671; herein SEQ ID NO: 1351), CAGGTCTTCACGGACTCAGACTATCAG (SEQ ID NO: 57 and 78 of WO2017100671; herein SEQ ID NO: 1352), CAAGTAAAACCTCTACAAATGTGGTAAAATCG (SEQ ID NO: 58 of WO2017100671; herein SEQ ID NO: 1353), ACTCATCGACCAATACTTGTACTATCTCTCTAGAAC (SEQ ID NO: 59 of WO2017100671; herein SEQ ID NO: 1354), GGAAGTATTCCTTGGTTTTGAACCCA (SEQ ID NO: 60 of WO2017100671; herein SEQ ID NO: 1355), GGTCGCGGTTCTTGTTTGTGGAT (SEQ ID NO: 61 of WO2017100671; herein SEQ ID NO: 1356), CGACCTTGAAGCGCATGAACTCCT (SEQ ID NO: 62 of WO2017100671; herein SEQ ID NO: 1357), GTATTCCTTGGTTTTGAACCCAACCGGTCTGCGCCTGTGCMNNMNNMNNMNNMNNMN NMNNTTGGGCACTCTGGTGGTTTGTC (SEQ ID NO: 63 of WO2017100671 wherein N may be A, C, T. or G; herein SEQ ID NO: 1358), GTATTCCTTGGTTTTGAACCCAACCGGTCTGCGCMNNMNNMNNAAAAGGCACCGCCAA AGTTTG (SEQ ID NO: 69 of WO2017100671 wherein N may be A, C, T, or G; herein SEQ ID NO: 1359), GTATTCCTTGGTTTTGAACCCAACCGGTCTGCGCCTGTGCMNNMNNMNNCACCGCCAA AGTTTGGGCACT (SEQ ID NO: 70 of WO2017100671 wherein N may be A, C, T, or G; herein SEQ ID NO: 1360), GTATTCCTTGGTTTTGAACCCAACCGGTCTGCGCCTGTGCCTTAAAMNNMNNMNNCAAA GTTTGGGCACTCTGGTGG (SEQ ID NO: 71 of WO2017100671 wherein N may be A, C, T, or G; herein SEQ ID NO: 1361), GTATTCCTTGGTTTTGAACCCAACCGGTCTGCGCCTGTGCCTTAAAAGGCACMNNMNNM NNTTGGGCACTCTGGTGGTTTGTG (SEQ ID NO: 72 of WO2017100671 wherein N may be A, C, T, or G; herein SEQ ID NO: 1362), ACTTTGGCGGTGCCTTTTAAG (SEQ ID NO: 74 of WO2017100671; herein SEQ ID NO: 1279), AGTGTGAGTAAGCCTTTTTTG (SEQ ID NO: 75 of WO2017100671; herein SEQ ID NO: 1280), TTTACGTTGACGACGCCTAAG (SEQ ID NO: 76 of WO2017100671; herein SEQ ID NO: 1281), TATACTTTGTCGCAGGGTTGG (SEQ ID NO: 77 of WO2017100671; herein SEQ ID NO: 1287), or CTTGCGAAGGAGCGGCTTTCG (SEQ ID NO: 79 of WO2017100671; herein SEQ ID NO: 1363).

In some embodiments, the AAV serotype may be, or may have a sequence as described in U.S. Pat. No. 9,624,274, the contents of which are herein incorporated by reference in their entirety, such as, but not limited to, AAV1 (SEQ ID NO: 181 of U.S. Pat. No. 9,624,274), AAV6 (SEQ ID NO: 182 of U.S. Pat. No. 9,624,274), AAV2 (SEQ ID NO: 183 of U.S. Pat. No. 9,624,274), AAV3b (SEQ ID NO: 184 of U.S. Pat. No. 9,624,274), AAV7 (SEQ ID NO: 185 of U.S. Pat. No. 9,624,274), AAV8 (SEQ ID NO: 186 of U.S. Pat. No. 9,624,274), AAV10 (SEQ ID NO: 187 of U.S. Pat. No. 9,624,274), AAV4 (SEQ ID NO: 188 of U.S. Pat. No. 9,624,274), AAV11 (SEQ ID NO: 189 of U.S. Pat. No. 9,624,274), bAAV (SEQ ID NO: 190 of U.S. Pat. No. 9,624,274), AAV5 (SEQ ID NO: 191 of U.S. Pat. No. 9,624,274), GPV (SEQ ID NO: 192 of U.S. Pat. No. 9,624,274; herein SEQ ID NO: 879), B19 (SEQ ID NO: 193 of U.S. Pat. No. 9,624,274; herein SEQ ID NO: 880), MVM (SEQ ID NO: 194 of U.S. Pat. No. 9,624,274; herein SEQ ID NO: 881), FPV (SEQ ID NO: 195 of U.S. Pat. No. 9,624,274; herein SEQ ID NO: 882), CPV (SEQ ID NO: 196 of U.S. Pat. No. 9,624,274; herein SEQ ID NO: 883) or variants thereof. Further, any of the structural protein inserts described in U.S. Pat. No. 9,624,274, may be inserted into, but not limited to, 1-453 and 1-587 of any parent AAV serotype, such as, but not limited to, AAV2 (SEQ ID NO: 183 of U.S. Pat. No. 9,624,274). The amino acid insert may be, but is not limited to, any of the following amino acid sequences, VNLTWSRASG (SEQ ID NO: 50 of U.S. Pat. No. 9,624,274; herein SEQ ID NO: 1364), EFCINHRGYWVCGD (SEQ ID NO:55 of U.S. Pat. No. 9,624,274; herein SEQ ID NO: 1365), EDGQVMDVDLS (SEQ ID NO: 85 of U.S. Pat. No. 9,624,274; herein SEQ ID NO: 1366), EKQRNGTLT (SEQ ID NO: 86 of U.S. Pat. No. 9,624,274; herein SEQ ID NO: 1367), TYQCRVTHPHLPRALMR (SEQ ID NO: 87 of U.S. Pat. No. 9,624,274; herein SEQ ID NO: 1368), RHSTTQPRKTKGSG (SEQ ID NO: 88 of U.S. Pat. No. 9,624,274; herein SEQ ID NO: 1369), DSNPRGVSAYLSR (SEQ ID NO: 89 of U.S. Pat. No. 9,624,274; herein SEQ ID NO: 1370), TITCLWDLAPSK (SEQ ID NO: 90 of U.S. Pat. No. 9,624,274; herein SEQ ID NO: 1371), KTKGSGFFVF (SEQ ID NO: 91 of U.S. Pat. No. 9,624,274; herein SEQ ID NO: 1372), THPHLPRALMRS (SEQ ID NO: 92 of U.S. Pat. No. 9,624,274; herein SEQ ID NO: 1373), GETYQCRVTHPHLPRALMRSTTK (SEQ ID NO: 93 of U.S. Pat. No. 9,624,274; herein SEQ ID NO: 1374), LPRALMRS (SEQ ID NO: 94 of U.S. Pat. No. 9,624,274; herein SEQ ID NO: 1375), INHRGYWV (SEQ ID NO: 95 of U.S. Pat. No. 9,624,274; herein SEQ ID NO: 1376), CDAGSVRTNAPD (SEQ ID NO: 60 of U.S. Pat. No. 9,624,274; herein SEQ ID NO: 1377), AKAVSNLTESRSESLQS (SEQ ID NO: 96 of U.S. Pat. No. 9,624,274; herein SEQ ID NO: 1378), SLTGDEFKKVLET (SEQ ID NO: 97 of U.S. Pat. No. 9,624,274; herein SEQ ID NO: 1379), REAVAYRFEED (SEQ ID NO: 98 of U.S. Pat. No. 9,624,274; herein SEQ ID NO: 1380), INPEIITLDG (SEQ ID NO: 99 of U.S. Pat. No. 9,624,274; herein SEQ ID NO: 1381), DISVTGAPVITATYL (SEQ ID NO: 100 of U.S. Pat. No. 9,624,274; herein SEQ ID NO: 1382), DISVTGAPVITA (SEQ ID NO: 101 of U.S. Pat. No. 9,624,274; herein SEQ ID NO: 1383), PKTVSNLTESSSESVQS (SEQ ID NO: 102 of U.S. Pat. No. 9,624,274; herein SEQ ID NO: 1384), SLMGDEFKAVLET (SEQ ID NO: 103 of U.S. Pat. No. 9,624,274; herein SEQ ID NO: 1385). QHSVAYTFEED (SEQ ID NO: 104 of U.S. Pat. No. 9,624,274; herein SEQ ID NO: 1386), INPEIITRDG (SEQ ID NO: 105 of U.S. Pat. No. 9,624,274; herein SEQ ID NO: 1387), DISLTGDPVITASYL (SEQ ID NO: 106 of U.S. Pat. No. 9,624,274; herein SEQ ID NO: 1388), DISLTGDPVITA (SEQ ID NO: 107 of U.S. Pat. No. 9,624,274; herein SEQ ID NO: 1389), DQSIDFEIDSA (SEQ ID NO: 108 of U.S. Pat. No. 9,624,274; herein SEQ ID NO: 1390), KNVSEDLPLPTFSPTLLGDS (SEQ ID NO: 109 of U.S. Pat. No. 9,624,274; herein SEQ ID NO: 1391), KNVSEDLPLPT (SEQ ID NO: 110 of U.S. Pat. No. 9,624,274; herein SEQ ID NO: 1392), CDSGRVRTDAPD (SEQ ID NO: 111 of U.S. Pat. No. 9,624,274; herein SEQ ID NO: 1393), FPEHLLVDFLQSLS (SEQ ID NO: 112 of U.S. Pat. No. 9,624,274; herein SEQ ID NO: 1394), DAEFRHDSG (SEQ ID NO: 65 of U.S. Pat. No. 9,624,274; herein SEQ ID NO: 1395), HYAAAQWDFGNTMCQL (SEQ ID NO: 113 of U.S. Pat. No. 9,624,274; herein SEQ ID NO: 1396), YAAQWDFGNTMCQ (SEQ ID NO: 114 of U.S. Pat. No. 9,624,274; herein SEQ ID NO: 1397), RSQKEGLHYT (SEQ ID NO: 115 of U.S. Pat. No. 9,624,274; herein SEQ ID NO: 1398), SSRTPSDKPVAHWANPQAE (SEQ ID NO: 116 of U.S. Pat. No. 9,624,274; herein SEQ ID NO: 1399), SRTPSDKPVAHWANP (SEQ ID NO: 117 of U.S. Pat. No. 9,624,274; herein SEQ ID NO: 1400), SSRTPSDKP (SEQ ID NO: 118 of U.S. Pat. No. 9,624,274; herein SEQ ID NO: 1401), NADGNVDYHMNSVP (SEQ ID NO: 119 of U.S. Pat. No. 9,624,274; herein SEQ ID NO: 1402), DGNVDYHMNSV (SEQ ID NO: 120 of U.S. Pat. No. 9,624,274; herein SEQ ID NO: 1403), RSFKEFLQSSLRALRQ (SEQ ID NO: 121 of U.S. Pat. No. 9,624,274; herein SEQ ID NO: 1404); FKEFLQSSLRA (SEQ ID NO: 122 of U.S. Pat. No. 9,624,274; herein SEQ ID NO: 1405), or QMWAPQWGPD (SEQ ID NO: 123 of U.S. Pat. No. 9,624,274; herein SEQ ID NO: 1406).

In some embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not be limited to, the amino acid sequence RGNRQA (SEQ ID NO: 3 of U.S. Pat. No. 9,475,845; herein SEQ ID NO: 1407), SSSTDP (SEQ ID NO: 4 of U.S. Pat. No. 9,475,845; herein SEQ ID NO: 1408). SSNTAP (SEQ ID NO: 5 of U.S. Pat. No. 9,475,845; herein SEQ ID NO: 1409), SNSNLP (SEQ ID NO: 6 of U.S. Pat. No. 9,475,845; herein SEQ ID NO: 1410), SSTTAP (SEQ ID NO: 7 of U.S. Pat. No. 9,475,845; herein SEQ ID NO: 1411), AANTAA (SEQ ID NO: 8 of U.S. Pat. No. 9,475,845; herein SEQ ID NO: 1412), QQNTAP (SEQ ID NO: 9 of U.S. Pat. No. 9,475,845; herein SEQ ID NO: 1413), SAQAQA (SEQ ID NO: 10 of U.S. Pat. No. 9,475,845; herein SEQ ID NO: 1414), QANTGP (SEQ ID NO: 11 of U.S. Pat. No. 9,475,845; herein SEQ ID NO: 1415), NATTAP (SEQ ID NO: 12 of U.S. Pat. No. 9,475,845; herein SEQ ID NO: 1416), SSTAGP (SEQ ID NO: 13 and 20 of U.S. Pat. No. 9,475,845; herein SEQ ID NO: 1417), QQNTAA (SEQ ID NO: 14 of U.S. Pat. No. 9,475,845; herein SEQ ID NO: 1418), PSTAGP (SEQ ID NO: 15 of U.S. Pat. No. 9,475,845; herein SEQ ID NO: 1419), NQNTAP (SEQ ID NO: 16 of U.S. Pat. No. 9,475,845; herein SEQ ID NO: 1420), QAANAP (SEQ ID NO: 17 of U.S. Pat. No. 9,475,845; herein SEQ ID NO: 1421), SIVGLP (SEQ ID NO: 18 of U.S. Pat. No. 9,475,845; herein SEQ ID NO: 1422), AASTAA (SEQ ID NO: 19, and 27 of U.S. Pat. No. 9,475,845; herein SEQ ID NO: 1423), SQNTTA (SEQ ID NO: 21 of U.S. Pat. No. 9,475,845; herein SEQ ID NO: 1424), QQDTAP (SEQ ID NO: 22 of U.S. Pat. No. 9,475,845; herein SEQ ID NO: 1425), QTNTGP (SEQ ID NO: 23 of U.S. Pat. No. 9,475,845; herein SEQ ID NO: 1426), QTNGAP (SEQ ID NO: 24 of U.S. Pat. No. 9,475,845; herein SEQ ID NO: 1427), QQNAAP (SEQ ID NO: 25 of U.S. Pat. No. 9,475,845; herein SEQ ID NO: 1428), or AANTQA (SEQ ID NO: 26 of U.S. Pat. No. 9,475,845; herein SEQ ID NO: 1429). In some embodiments, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences, NGRAHA (SEQ ID NO: 38 of U.S. Pat. No. 9,475,845; herein SEQ ID NO: 1430), QPEHSST (SEQ ID NO: 39 and 50 of U.S. Pat. No. 9,475,845; herein SEQ ID NO: 1431). VNTANST (SEQ ID NO: 40 of U.S. Pat. No. 9,475,845; herein SEQ ID NO: 1432), HGPMQKS (SEQ ID NO: 41 of U.S. Pat. No. 9,475,845; herein SEQ ID NO: 1433). PHKPPLA (SEQ ID NO: 42 of U.S. Pat. No. 9,475,845; herein SEQ ID NO: 1434), IKNNEMW (SEQ ID NO: 43 of U.S. Pat. No. 9,475,845; herein SEQ ID NO: 1435), RNLDTPM (SEQ ID NO: 44 of U.S. Pat. No. 9,475,845; herein SEQ ID NO: 1436), VDSHRQS (SEQ ID NO: 45 of U.S. Pat. No. 9,475,845; herein SEQ ID NO: 1437), YDSKTKT (SEQ ID NO: 46 of U.S. Pat. No. 9,475,845; herein SEQ ID NO: 1438), SQLPHQK (SEQ ID NO: 47 of U.S. Pat. No. 9,475,845; herein SEQ ID NO: 1439), STMQQNT (SEQ ID NO: 48 of U.S. Pat. No. 9,475,845; herein SEQ ID NO: 1440), TERYMTQ (SEQ ID NO: 49 of U.S. Pat. No. 9,475,845; herein SEQ ID NO: 1441), DASLSTS (SEQ ID NO: 51 of U.S. Pat. No. 9,475,845; herein SEQ ID NO: 1442), DLPNKKT (SEQ ID NO: 52 of U.S. Pat. No. 9,475,845; herein SEQ ID NO: 1443), DLTAARL (SEQ ID NO: 53 of U.S. Pat. No. 9,475,845; hercin SEQ ID NO: 1444), EPHQFNY (SEQ ID NO: 54 of U.S. Pat. No. 9,475,845; herein SEQ ID NO: 1445), EPQSNHT (SEQ ID NO: 55 of U.S. Pat. No. 9,475,845; herein SEQ ID NO: 1446), MSSWPSQ (SEQ ID NO: 56 of U.S. Pat. No. 9,475,845; herein SEQ ID NO: 1447), NPKHNAT (SEQ ID NO: 57 of U.S. Pat. No. 9,475,845; herein SEQ ID NO: 1448), PDGMRTT (SEQ ID NO: 58 of U.S. Pat. No. 9,475,845; herein SEQ ID NO: 1449), PNNNKTT (SEQ ID NO: 59 of U.S. Pat. No. 9,475,845; herein SEQ ID NO: 1450), QSTTHDS (SEQ ID NO: 60 of U.S. Pat. No. 9,475,845; herein SEQ ID NO: 1451), TGSKQKQ (SEQ ID NO: 61 of U.S. Pat. No. 9,475,845; herein SEQ ID NO: 1452), SLKHQAL (SEQ ID NO: 62 of U.S. Pat. No. 9,475,845; herein SEQ ID NO: 1453), SPIDGEQ (SEQ ID NO: 63 of U.S. Pat. No. 9,475,845; herein SEQ ID NO: 1454), WIFPWIQL (SEQ ID NO: 64 and 112 of U.S. Pat. No. 9,475,845; herein SEQ ID NO: 1455), CDCRGDCFC (SEQ ID NO: 65 of U.S. Pat. No. 9,475,845; herein SEQ ID NO: 1456), CNGRC (SEQ ID NO: 66 of U.S. Pat. No. 9,475,845; herein SEQ ID NO: 1457), CPRECES (SEQ ID NO: 67 of U.S. Pat. No. 9,475,845; herein SEQ ID NO: 1458), CTTHWGFTLC (SEQ ID NO: 68 and 123 of U.S. Pat. No. 9,475,845; herein SEQ ID NO: 1459), CGRRAGGSC (SEQ ID NO: 69 of U.S. Pat. No. 9,475,845; herein SEQ ID NO: 1460), CKGGRAKDC (SEQ ID NO: 70 of U.S. Pat. No. 9,475,845; herein SEQ ID NO: 1461), CVPELGHEC (SEQ ID NO: 71 and 115 of U.S. Pat. No. 9,475,845; herein SEQ ID NO: 1462), CRRETAWAK (SEQ ID NO: 72 of U.S. Pat. No. 9,475,845; herein SEQ ID NO: 1463), VSWFSHRYSPFAVS (SEQ ID NO: 73 of U.S. Pat. No. 9,475,845; herein SEQ ID NO: 1464), GYRDGYAGPILYN (SEQ ID NO: 74 of U.S. Pat. No. 9,475,845; herein SEQ ID NO: 1465). XXXYXXX (SEQ ID NO: 75 of U.S. Pat. No. 9,475,845; herein SEQ ID NO: 1466), YXNW (SEQ ID NO: 76 of U.S. Pat. No. 9,475,845; herein SEQ ID NO: 1467), RPLPPLP (SEQ ID NO: 77 of U.S. Pat. No. 9,475,845; herein SEQ ID NO: 1468), APPLPPR (SEQ ID NO: 78 of U.S. Pat. No. 9,475,845; herein SEQ ID NO: 1469), DVFYPYPYASGS (SEQ ID NO: 79 of U.S. Pat. No. 9,475,845; herein SEQ ID NO: 1470), MYWYPY (SEQ ID NO: 80 of U.S. Pat. No. 9,475,845; herein SEQ ID NO: 1471), DITWDQLWDLMK (SEQ ID NO: 81 of U.S. Pat. No. 9,475,845; herein SEQ ID NO: 1472), CWDDXWLC (SEQ ID NO: 82 of U.S. Pat. No. 9,475,845; herein SEQ ID NO: 1473), EWCEYLGGYLRCYA (SEQ ID NO: 83 of U.S. Pat. No. 9,475,845; herein SEQ ID NO: 1474), YXCXXGPXTWXCXP (SEQ ID NO: 84 of U.S. Pat. No. 9,475,845; herein SEQ ID NO: 1475), IEGPTLRQWLAARA (SEQ ID NO: 85 of U.S. Pat. No. 9,475,845; herein SEQ ID NO: 1476), LWXXX (SEQ ID NO: 86 of U.S. Pat. No. 9,475,845; herein SEQ ID NO: 1477), XFXXYLW (SEQ ID NO: 87 of U.S. Pat. No. 9,475,845; herein SEQ ID NO: 1478), SSIISHFRWGLCD (SEQ ID NO: 88 of U.S. Pat. No. 9,475,845; herein SEQ ID NO: 1479), MSRPACPPNDKYE (SEQ ID NO: 89 of U.S. Pat. No. 9,475,845; herein SEQ ID NO: 1480), CLRSGRGC (SEQ ID NO: 90 of U.S. Pat. No. 9,475,845; herein SEQ ID NO: 1481), CHWMFSPWC (SEQ ID NO: 91 of U.S. Pat. No. 9,475,845; herein SEQ ID NO: 1482), WXXF (SEQ ID NO: 92 of U.S. Pat. No. 9,475,845; herein SEQ ID NO: 1483), CSSRLDAC (SEQ ID NO: 93 of U.S. Pat. No. 9,475,845; herein SEQ ID NO: 1484), CLPVASC (SEQ ID NO: 94 of U.S. Pat. No. 9,475,845; herein SEQ ID NO: 1485), CGFECVRQCPERC (SEQ ID NO: 95 of U.S. Pat. No. 9,475,845; herein SEQ ID NO: 1486), CVALCREACGEGC (SEQ ID NO: 96 of U.S. Pat. No. 9,475,845; herein SEQ ID NO: 1487), SWCEPGWCR (SEQ ID NO: 97 of U.S. Pat. No. 9,475,845; herein SEQ ID NO: 1488), YSGKWGW (SEQ ID NO: 98 of U.S. Pat. No. 9,475,845; herein SEQ ID NO: 1489), GLSGGRS (SEQ ID NO: 99 of U.S. Pat. No. 9,475,845; herein SEQ ID NO: 1490). LMLPRAD (SEQ ID NO: 100 of U.S. Pat. No. 9,475,845; herein SEQ ID NO: 1491). CSCFRDVCC (SEQ ID NO: 101 of U.S. Pat. No. 9,475,845; herein SEQ ID NO: 1492), CRDVVSVIC (SEQ ID NO: 102 of U.S. Pat. No. 9,475,845; herein SEQ ID NO: 1493), MARSGL (SEQ ID NO: 103 of U.S. Pat. No. 9,475,845; herein SEQ ID NO: 1494), MARAKE (SEQ ID NO: 104 of U.S. Pat. No. 9,475,845; herein SEQ ID NO: 1495), MSRTMS (SEQ ID NO: 105 of U.S. Pat. No. 9,475,845; herein SEQ ID NO: 1496, KCCYSL (SEQ ID NO: 106 of U.S. Pat. No. 9,475,845; herein SEQ ID NO: 1497), MYWGDSHWLQYWYE (SEQ ID NO: 107 of U.S. Pat. No. 9,475,845; herein SEQ ID NO: 1498), MQLPLAT (SEQ ID NO: 108 of U.S. Pat. No. 9,475,845; herein SEQ ID NO: 1499), EWLS (SEQ ID NO: 109 of U.S. Pat. No. 9,475,845; herein SEQ ID NO: 1500), SNEW (SEQ ID NO: 110 of U.S. Pat. No. 9,475,845; herein SEQ ID NO: 1501), TNYL (SEQ ID NO: 111 of U.S. Pat. No. 9,475,845; herein SEQ ID NO: 1502), WDLAWMFRLPVG (SEQ ID NO: 113 of U.S. Pat. No. 9,475,845; herein SEQ ID NO: 1503), CTVALPGGYVRVC (SEQ ID NO: 114 of U.S. Pat. No. 9,475,845; herein SEQ ID NO: 1504), CVAYCIEHHCWTC (SEQ ID NO: 116 of U.S. Pat. No. 9,475,845; herein SEQ ID NO: 1505), CVFAHNYDYLVC (SEQ ID NO: 117 of U.S. Pat. No. 9,475,845; herein SEQ ID NO: 1506), CVFTSNYAFC (SEQ ID NO: 118 of U.S. Pat. No. 9,475,845; herein SEQ ID NO: 1507), VHSPNKK (SEQ ID NO: 119 of U.S. Pat. No. 9,475,845; herein SEQ ID NO: 1508), CRGDGWC (SEQ ID NO: 120 of U.S. Pat. No. 9,475,845; herein SEQ ID NO: 1509), XRGCDX (SEQ ID NO: 121 of U.S. Pat. No. 9,475,845; herein SEQ ID NO: 1510). PXXX (SEQ ID NO: 122 of U.S. Pat. No. 9,475,845; herein SEQ ID NO: 1511). SGKGPRQITAL (SEQ ID NO: 124 of U.S. Pat. No. 9,475,845; herein SEQ ID NO: 1512), AAAAAAAAAXXXXX (SEQ ID NO: 125 of U.S. Pat. No. 9,475,845; herein SEQ ID NO: 1513), VYMSPF (SEQ ID NO: 126 of U.S. Pat. No. 9,475,845; herein SEQ ID NO: 1514), ATWLPPR (SEQ ID NO: 127 of U.S. Pat. No. 9,475,845; herein SEQ ID NO: 1515), HTMYYHHYQHHL (SEQ ID NO: 128 of U.S. Pat. No. 9,475,845; herein SEQ ID NO: 1516), SEVGCRAGPLQWLCEKYFG (SEQ ID NO: 129 of U.S. Pat. No. 9,475,845; herein SEQ ID NO: 1517), CGLLPVGRPDRNVWRWLC (SEQ ID NO: 130 of U.S. Pat. No. 9,475,845; herein SEQ ID NO: 1518), CKGQCDRFKGLPWEC (SEQ ID NO: 131 of U.S. Pat. No. 9,475,845; herein SEQ ID NO: 1519), SGRSA (SEQ ID NO: 132 of U.S. Pat. No. 9,475,845; herein SEQ ID NO: 1520). WGFP (SEQ ID NO: 133 of U.S. Pat. No. 9,475,845; herein SEQ ID NO: 1521), AEPMPHSLNFSQYLWYT (SEQ ID NO: 134 of U.S. Pat. No. 9,475,845; herein SEQ ID NO: 1522), WAYXSP (SEQ ID NO: 135 of U.S. Pat. No. 9,475,845; herein SEQ ID NO: 1523), IELLQAR (SEQ ID NO: 136 of U.S. Pat. No. 9,475,845; herein SEQ ID NO: 1524), AYTKCSRQWRTCMTTH (SEQ ID NO: 137 of U.S. Pat. No. 9,475,845; herein SEQ ID NO: 1525), PQNSKIPGPTFLDPH (SEQ ID NO: 138 of U.S. Pat. No. 9,475,845; herein SEQ ID NO: 1526), SMEPALPDWWWKMFK (SEQ ID NO: 139 of U.S. Pat. No. 9,475,845; herein SEQ ID NO: 1527), ANTPCGPYTHDCPVKR (SEQ ID NO: 140 of U.S. Pat. No. 9,475,845; herein SEQ ID NO: 1528), TACHQHVRMVRP (SEQ ID NO: 141 of U.S. Pat. No. 9,475,845; herein SEQ ID NO: 1529), VPWMEPAYQRFL (SEQ ID NO: 142 of U.S. Pat. No. 9,475,845; herein SEQ ID NO: 1530), DPRATPGS (SEQ ID NO: 143 of U.S. Pat. No. 9,475,845; herein SEQ ID NO: 1531), FRPNRAQDYNTN (SEQ ID NO: 144 of U.S. Pat. No. 9,475,845; herein SEQ ID NO: 1532), CTKNSYLMC (SEQ ID NO: 145 of U.S. Pat. No. 9,475,845; herein SEQ ID NO: 1533), CXXTXXXGXGC (SEQ ID NO: 146 of U.S. Pat. No. 9,475,845; herein SEQ ID NO: 1534), CPIEDRPMC (SEQ ID NO: 147 of U.S. Pat. No. 9,475,845; herein SEQ ID NO: 1535), HEWSYLAPYPWF (SEQ ID NO: 148 of U.S. Pat. No. 9,475,845; herein SEQ ID NO: 1536), MCPKHPLGC (SEQ ID NO: 149 of U.S. Pat. No. 9,475,845; herein SEQ ID NO: 1537), RMWPSSTVNLSAGRR (SEQ ID NO: 150 of U.S. Pat. No. 9,475,845; herein SEQ ID NO: 1538), SAKTAVSQRVWLPSHRGGEP (SEQ ID NO: 151 of U.S. Pat. No. 9,475,845; herein SEQ ID NO: 1539), KSREHVNNSACPSKRITAAL (SEQ ID NO: 152 of U.S. Pat. No. 9,475,845; herein SEQ ID NO: 1540), EGFR (SEQ ID NO: 153 of U.S. Pat. No. 9,475,845; herein SEQ ID NO: 1541), AGLGVR (SEQ ID NO: 154 of U.S. Pat. No. 9,475,845; herein SEQ ID NO: 1542), GTRQGHTMRLGVSDG (SEQ ID NO: 155 of U.S. Pat. No. 9,475,845; herein SEQ ID NO: 1543), IAGLATPGWSHWLAL (SEQ ID NO: 156 of U.S. Pat. No. 9,475,845; herein SEQ ID NO: 1544), SMSIARL (SEQ ID NO: 157 of U.S. Pat. No. 9,475,845; herein SEQ ID NO: 1545), HTFEPGV (SEQ ID NO: 158 of U.S. Pat. No. 9,475,845; herein SEQ ID NO: 1546). NTSLKRISNKRIRRK (SEQ ID NO: 159 of U.S. Pat. No. 9,475,845; herein SEQ ID NO: 1547). LRIKRKRRKRKKTRK (SEQ ID NO: 160 of U.S. Pat. No. 9,475,845; herein SEQ ID NO: 1548), GGG, GFS, LWS, EGG, LLV, LSP, LBS, AGG, GRR, GGH and GTV.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 1549) or variants thereof, wherein the specific site is at least one site selected from sites R447. G453, S578, N587, N587+1, S662 of VP1 or fragment thereof.

Further, any of the mutated sequences described in US 20160369298, may be or may have, but not limited to, any of the following sequences SDSGASN (SEQ ID NO: 1 and SEQ ID NO: 231 of US20160369298; herein SEQ ID NO: 1550), SPSGASN (SEQ ID NO: 2 of US20160369298; herein SEQ ID NO: 1551), SHSGASN (SEQ ID NO: 3 of US20160369298; herein SEQ ID NO: 1552), SRSGASN (SEQ ID NO: 4 of US20160369298; herein SEQ ID NO: 1553), SKSGASN (SEQ ID NO: 5 of US20160369298; herein SEQ ID NO: 1554), SNSGASN (SEQ ID NO: 6 of US20160369298; herein SEQ ID NO: 1555), SGSGASN (SEQ ID NO: 7 of US20160369298; herein SEQ ID NO: 1556), SASGASN (SEQ ID NO: 8, 175, and 221 of US20160369298; herein SEQ ID NO: 1557), SESGTSN (SEQ ID NO: 9 of US20160369298; herein SEQ ID NO: 1558), STTGGSN (SEQ ID NO: 10 of US20160369298; herein SEQ ID NO: 1559), SSAGSTN (SEQ ID NO: 11 of US20160369298; herein SEQ ID NO: 1560), NNDSQA (SEQ ID NO: 12 of US20160369298; herein SEQ ID NO: 1561), NNRNQA (SEQ ID NO: 13 of US20160369298; herein SEQ ID NO: 1562), NNNKQA (SEQ ID NO: 14 of US20160369298; herein SEQ ID NO: 1563). NAKRQA (SEQ ID NO: 15 of US20160369298; herein SEQ ID NO: 1564), NDEHQA (SEQ ID NO: 16 of US20160369298; herein SEQ ID NO: 1565), NTSQKA (SEQ ID NO: 17 of US20160369298; herein SEQ ID NO: 1566), YYLSRTNTPSGTDTQSRLVFSQAGA (SEQ ID NO: 18 of US20160369298; herein SEQ ID NO: 1567), YYLSRTNTDSGTETQSGLDFSQAGA (SEQ ID NO: 19 of US20160369298; herein SEQ ID NO: 1568), YYLSRTNTESGTPTQSALEFSQAGA (SEQ ID NO: 20 of US20160369298; herein SEQ ID NO: 1569), YYLSRTNTHSGTHTQSPLHFSQAGA (SEQ ID NO: 21 of US20160369298; herein SEQ ID NO: 1570), YYLSRTNTSSGTITISHLIFSQAGA (SEQ ID NO: 22 of US20160369298; herein SEQ ID NO: 1571), YYLSRTNTRSGIMTKSSLMFSQAGA (SEQ ID NO: 23 of US20160369298; herein SEQ ID NO: 1572), YYLSRTNTKSGRKTLSNLSFSQAGA (SEQ ID NO: 24 of US20160369298; herein SEQ ID NO: 1573), YYLSRTNDGSGPVTPSKLRFSQRGA (SEQ ID NO: 25 of US20160369298; herein SEQ ID NO: 1574), YYLSRTNAASGHATHSDLKFSQPGA (SEQ ID NO: 26 of US20160369298; herein SEQ ID NO: 1575), YYLSRTNGQAGSLTMSELGFSQVGA (SEQ ID NO: 27 of US20160369298; herein SEQ ID NO: 1576), YYLSRTNSTGGNQTTSQLLFSQLSA (SEQ ID NO: 28 of US20160369298; herein SEQ ID NO: 1577), YFLSRTNNNTGLNTNSTLNFSQGRA (SEQ ID NO: 29 of US20160369298; herein SEQ ID NO: 1578), SKTGADNNNSEYSWTG (SEQ ID NO: 30 of US20160369298; herein SEQ ID NO: 1579), SKTDADNNNSEYSWTG (SEQ ID NO: 31 of US20160369298; herein SEQ ID NO: 1580), SKTEADNNNSEYSWTG (SEQ ID NO: 32 of US20160369298; herein SEQ ID NO: 1581), SKTPADNNNSEYSWTG (SEQ ID NO: 33 of US20160369298; herein SEQ ID NO: 1582), SKTHADNNNSEYSWTG (SEQ ID NO: 34 of US20160369298; herein SEQ ID NO: 1583), SKTQADNNNSEYSWTG (SEQ ID NO: 35 of US20160369298; herein SEQ ID NO: 1584), SKTIADNNNSEYSWTG (SEQ ID NO: 36 of US20160369298; herein SEQ ID NO: 1585), SKTMADNNNSEYSWTG (SEQ ID NO: 37 of US20160369298; herein SEQ ID NO: 1586), SKTRADNNNSEYSWTG (SEQ ID NO: 38 of US20160369298; herein SEQ ID NO: 1587), SKTNADNNNSEYSWTG (SEQ ID NO: 39 of US20160369298; herein SEQ ID NO: 1588). SKTVGRNNNSEYSWTG (SEQ ID NO: 40 of US20160369298; herein SEQ ID NO: 1589), SKTADRNNNSEYSWTG (SEQ ID NO: 41 of US20160369298; herein SEQ ID NO: 1590), SKKLSQNNNSKYSWQG (SEQ ID NO: 42 of US20160369298; herein SEQ ID NO: 1591), SKPTTGNNNSDYSWPG (SEQ ID NO: 43 of US20160369298; herein SEQ ID NO: 1592), STQKNENNNSNYSWPG (SEQ ID NO: 44 of US20160369298; herein SEQ ID NO: 1593), HKDDEGKF (SEQ ID NO: 45 of US20160369298; herein SEQ ID NO: 1594), HKDDNRKF (SEQ ID NO: 46 of US20160369298; herein SEQ ID NO: 1595), HKDDTNKF (SEQ ID NO: 47 of US20160369298; herein SEQ ID NO: 1596), HEDSDKNF (SEQ ID NO: 48 of US20160369298; herein SEQ ID NO: 1597), HRDGADSF (SEQ ID NO: 49 of US20160369298; herein SEQ ID NO: 1598), HGDNKSRF (SEQ ID NO: 50 of US20160369298; herein SEQ ID NO: 1599), KQGSEKTNVDFEEV (SEQ ID NO: 51 of US20160369298; herein SEQ ID NO: 1600), KQGSEKTNVDSEEV (SEQ ID NO: 52 of US20160369298; herein SEQ ID NO: 1601), KQGSEKTNVDVEEV (SEQ ID NO: 53 of US20160369298; herein SEQ ID NO: 1602), KQGSDKTNVDDAGV (SEQ ID NO: 54 of US20160369298; herein SEQ ID NO: 1603), KQGSSKTNVDPREV (SEQ ID NO: 55 of US20160369298; herein SEQ ID NO: 1604), KQGSRKTNVDHKQV (SEQ ID NO: 56 of US20160369298; herein SEQ ID NO: 1605), KQGSKGGNVDTNRV (SEQ ID NO: 57 of US20160369298; herein SEQ ID NO: 1606), KQGSGEANVDNGDV (SEQ ID NO: 58 of US20160369298; herein SEQ ID NO: 1607), KQDAAADNIDYDHV (SEQ ID NO: 59 of US20160369298; herein SEQ ID NO: 1608), KQSGTRSNAAASSV (SEQ ID NO: 60 of US20160369298; herein SEQ ID NO: 1609), KENTNTNDTELTNV (SEQ ID NO: 61 of US20160369298; herein SEQ ID NO: 1610), QRGNNVAATADVNT (SEQ ID NO: 62 of US20160369298; herein SEQ ID NO: 1611). QRGNNEAATADVNT (SEQ ID NO: 63 of US20160369298; herein SEQ ID NO: 1612), QRGNNPAATADVNT (SEQ ID NO: 64 of US20160369298; herein SEQ ID NO: 1613), QRGNNHAATADVNT (SEQ ID NO: 65 of US20160369298; herein SEQ ID NO: 1614), QEENNIAATPGVNT (SEQ ID NO: 66 of US20160369298; herein SEQ ID NO: 1615). QPPNNMAATHEVNT (SEQ ID NO: 67 of US20160369298; herein SEQ ID NO: 1616), QHHNNSAATTIVNT (SEQ ID NO: 68 of US20160369298; herein SEQ ID NO: 1617), QTTNNRAAFNMVET (SEQ ID NO: 69 of US20160369298; herein SEQ ID NO: 1618), QKKNNNAASKKVAT (SEQ ID NO: 70 of US20160369298; herein SEQ ID NO: 1619), QGGNNKAADDAVKT (SEQ ID NO: 71 of US20160369298; herein SEQ ID NO: 1620), QAAKGGAADDAVKT (SEQ ID NO: 72 of US20160369298; herein SEQ ID NO: 1621), QDDRAAAANESVDT (SEQ ID NO: 73 of US20160369298; herein SEQ ID NO: 1622), QQQHDDAAYQRVHT (SEQ ID NO: 74 of US20160369298; herein SEQ ID NO: 1623), QSSSSLAAVSTVQT (SEQ ID NO: 75 of US20160369298; herein SEQ ID NO: 1624), QNNQTTAAIRNVTT (SEQ ID NO: 76 of US20160369298; herein SEQ ID NO: 1625), NYNKKSDNVDFT (SEQ ID NO: 77 of US20160369298; herein SEQ ID NO: 1626), NYNKKSENVDFT (SEQ ID NO: 78 of US20160369298; herein SEQ ID NO: 1627), NYNKKSLNVDFT (SEQ ID NO: 79 of US20160369298; herein SEQ ID NO: 1628), NYNKKSPNVDFT (SEQ ID NO: 80 of US20160369298; herein SEQ ID NO: 1629), NYSKKSHCVDFT (SEQ ID NO: 81 of US20160369298; herein SEQ ID NO: 1630), NYRKTIYVDFT (SEQ ID NO: 82 of US20160369298; herein SEQ ID NO: 1631), NYKEKKDVHFT (SEQ ID NO: 83 of US20160369298; herein SEQ ID NO: 1632), NYGHRAIVQFT (SEQ ID NO: 84 of US20160369298; herein SEQ ID NO: 1633), NYANHQFVVCT (SEQ ID NO: 85 of US20160369298; herein SEQ ID NO: 1634), NYDDDPTGVLLT (SEQ ID NO: 86 of US20160369298; herein SEQ ID NO: 1635), NYDDPTGVLLT (SEQ ID NO: 87 of US20160369298; herein SEQ ID NO: 1636), NFEQQNSVEWT (SEQ ID NO: 88 of US20160369298; herein SEQ ID NO: 1637), SQSGASN (SEQ ID NO: 89 and SEQ ID NO: 241 of US20160369298; herein SEQ ID NO: 1638), NNGSQA (SEQ ID NO: 90 of US20160369298; herein SEQ ID NO: 1639), YYLSRTNTPSGTTTWSRLQFSQAGA (SEQ ID NO: 91 of US20160369298; herein SEQ ID NO: 1640), SKTSADNNNSEYSWTG (SEQ ID NO: 92 of US20160369298; herein SEQ ID NO: 1641), HKDDEEKF (SEQ ID NO: 93, 209, 214, 219, 224, 234, 239, and 244 of US20160369298; herein SEQ ID NO: 1642), KQGSEKTNVDIEEV (SEQ ID NO: 94 of US20160369298; herein SEQ ID NO: 1643), QRGNNQAATADVNT (SEQ ID NO: 95 of US20160369298; herein SEQ ID NO: 1644), NYNKKSVNVDFT (SEQ ID NO: 96 of US20160369298; herein SEQ ID NO: 1645), SQSGASNYNTPSGTTTQSRLQFSTSADNNNSEYSWTGATKYH (SEQ ID NO: 106 of US20160369298; herein SEQ ID NO: 1646), SASGASNFNSEGGSLTQSSLGFSTDGENNNSDFSWTGATKYH (SEQ ID NO: 107 of US20160369298; herein SEQ ID NO: 1647), SQSGASNYNTPSGTTTQSRLQFSTDGENNNSDFSWTGATKYH (SEQ ID NO: 108 of US20160369298; herein SEQ ID NO: 1648), SASGASNYNTPSGTTTQSRLQFSTSADNNNSEFSWPGATTYH (SEQ ID NO: 109 of US20160369298; herein SEQ ID NO: 1649), SQSGASNFNSEGGSLTQSSLGFSTDGENNNSDFSWTGATKYH (SEQ ID NO: 110 of US20160369298; herein SEQ ID NO: 1650), SASGASNYNTPSGSLTQSSLGFSTDGENNNSDFSWTGATKYH (SEQ ID NO: 111 of US20160369298; herein SEQ ID NO: 1651), SQSGASNYNTPSGTTTQSRLQFSTSADNNNSDFSWTGATKYH (SEQ ID NO: 112 of US20160369298; herein SEQ ID NO: 1652), SGAGASNFNSEGGSLTQSSLGFSTDGENNNSDFSWTGATKYH (SEQ ID NO: 113 of US20160369298; herein SEQ ID NO: 1653), SGAGASN (SEQ ID NO: 176 of US20160369298; herein SEQ ID NO: 1654), NSEGGSLTQSSLGFS (SEQ ID NO: 177, 185, 193 and 202 of US20160369298; herein SEQ ID NO: 1655), TDGENNNSDFS (SEQ ID NO: 178 of US20160369298; herein SEQ ID NO: 1656), SEFSWPGATT (SEQ ID NO: 179 of US20160369298; herein SEQ ID NO: 1657), TSADNNNSDFSWT (SEQ ID NO: 180 of US20160369298; herein SEQ ID NO: 1658), SQSGASNY (SEQ ID NO: 181, 187, and 198 of US20160369298; herein SEQ ID NO: 1659), NTPSGTTTQSRLQFS (SEQ ID NO: 182, 188, 191, and 199 of US20160369298; herein SEQ ID NO: 1660), TSADNNNSEYSWTGATKYH (SEQ ID NO: 183 of US20160369298; herein SEQ ID NO: 1661), SASGASNF (SEQ ID NO: 184 of US20160369298; herein SEQ ID NO: 1662), TDGENNNSDFSWTGATKYH (SEQ ID NO: 186, 189, 194, 197, and 203 of US20160369298; herein SEQ ID NO: 1663). SASGASNY (SEQ ID NO: 190 and SEQ ID NO: 195 of US20160369298; herein SEQ ID NO: 1664), TSADNNNSEFSWPGATTYH (SEQ ID NO: 192 of US20160369298; herein SEQ ID NO: 1665), NTPSGSLTQSSLGFS (SEQ ID NO: 196 of US20160369298; herein SEQ ID NO: 1666), TSADNNNSDFSWTGATKYH (SEQ ID NO: 200 of US20160369298; herein SEQ ID NO: 1667), SGAGASNF (SEQ ID NO: 201 of US20160369298; herein SEQ ID NO: 1668), CTCCAGVVSVVSMRSRVCVNSGCAGCTDHCVVSRNSGTCVMSACACAA (SEQ ID NO: 204 of US20160369298; herein SEQ ID NO: 1669), CTCCAGAGAGGCAACAGACAAGCAGCTACCGCAGATGTCAACACACAA (SEQ ID NO: 205 of US20160369298; herein SEQ ID NO: 1670), SAAGASN (SEQ ID NO: 206 of US20160369298; herein SEQ ID NO: 1671), YFLSRTNTESGSTTQSTLRFSQAG (SEQ ID NO: 207 of US20160369298; herein SEQ ID NO: 1672), SKTSADNNNSDFS (SEQ ID NO: 208, 228, and 253 of US20160369298; herein SEQ ID NO: 1673), KQGSEKTDVDIDKV (SEQ ID NO: 210 of US20160369298; herein SEQ ID NO: 1674). STAGASN (SEQ ID NO: 211 of US20160369298; herein SEQ ID NO: 1675), YFLSRTNTTSGIETQSTLRFSQAG (SEQ ID NO: 212 and SEQ ID NO: 247 of US20160369298; herein SEQ ID NO: 1676), SKTDGENNNSDFS (SEQ ID NO: 213 and SEQ ID NO: 248 of US20160369298; herein SEQ ID NO: 1677), KQGAAADDVEIDGV (SEQ ID NO: 215 and SEQ ID NO: 250 of US20160369298; herein SEQ ID NO: 1678), SEAGASN (SEQ ID NO: 216 of US20160369298; herein SEQ ID NO: 1679), YYLSRTNTPSGTTTQSRLQFSQAG (SEQ ID NO: 217, 232 and 242 of US20160369298; herein SEQ ID NO: 1680), SKTSADNNNSEYS (SEQ ID NO: 218, 233, 238, and 243 of US20160369298; herein SEQ ID NO: 1681), KQGSEKTNVDIEKV (SEQ ID NO: 220, 225 and 245 of US20160369298; herein SEQ ID NO: 1682), YFLSRTNDASGSDTKSTLLFSQAG (SEQ ID NO: 222 of US20160369298; herein SEQ ID NO: 1683), STTPSENNNSEYS (SEQ ID NO: 223 of US20160369298; herein SEQ ID NO: 1684), SAAGATN (SEQ ID NO: 226 and SEQ ID NO: 251 of US20160369298; herein SEQ ID NO: 1685), YFLSRTNGEAGSATLSELRFSQAG (SEQ ID NO: 227 of US20160369298; herein SEQ ID NO: 1686), HGDDADRF (SEQ ID NO: 229 and SEQ ID NO: 254 of US20160369298; herein SEQ ID NO: 1687), KQGAEKSDVEVDRV (SEQ ID NO: 230 and SEQ ID NO: 255 of US20160369298; herein SEQ ID NO: 1688), KQDSGGDNIDIDQV (SEQ ID NO: 235 of US20160369298; herein SEQ ID NO: 1689), SDAGASN (SEQ ID NO: 236 of US20160369298; herein SEQ ID NO: 1690), YFLSRTNTEGGHDTQSTLRFSQAG (SEQ ID NO: 237 of US20160369298; herein SEQ ID NO: 1691), KEDGGGSDVAIDEV (SEQ ID NO: 240 of US20160369298; herein SEQ ID NO: 1692), SNAGASN (SEQ ID NO: 246 of US20160369298; herein SEQ ID NO: 1693), and YFLSRTNGEAGSATLSELRFSQPG (SEQ ID NO: 252 of US20160369298; herein SEQ ID NO: 1694). Non-limiting examples of nucleotide sequences that may encode the amino acid mutated sites include the following, AGCVVMDCAGGARSCASCAAC (SEQ ID NO: 97 of US20160369298; herein SEQ ID NO: 1695), AACRACRRSMRSMAGGCA (SEQ ID NO: 98 of US20160369298; herein SEQ ID NO: 1696), CACRRGGACRRCRMSRRSARSTTT (SEQ ID NO: 99 of US20160369298; herein SEQ ID NO: 1697), TATTTCTTGAGCAGAACAAACRVCVVSRSCGGAMNCVHSACGMHSTCAVVSCTTVDSTT TTCTCAGSBCRGSGCG (SEQ ID NO: 100 of US20160369298; herein SEQ ID NO: 1698), TCAAMAMMAVNSRVCSRSAACAACAACAGTRASTTCTCGTGGMMAGGA (SEQ ID NO: 101 of US20160369298; herein SEQ ID NO: 1699), AAGSAARRCRSCRVSRVARVCRATRYCGMSNHCRVMVRSGTC (SEQ ID NO: 102 of US20160369298; herein SEQ ID NO: 1700), CAGVVSVVSMRSRVCVNSGCAGCTDHCVVSRNSGTCVMSACA (SEQ ID NO: 103 of US20160369298; herein SEQ ID NO: 1701), AACTWCRVSVASMVSVHSDDTGTGSWSTKSACT (SEQ ID NO: 104 of US20160369298; herein SEQ ID NO: 1702). TTGTTGAACATCACCACGTGACGCACGTTC (SEQ ID NO: 256 of US20160369298; herein SEQ ID NO: 1703), TCCCCGTGGTTCTACTACATAATGTGGCCG (SEQ ID NO: 257 of US20160369298; herein SEQ ID NO: 1704), TTCCACACTCCGTTTTGGATAATGTTGAAC (SEQ ID NO: 258 of US20160369298; herein SEQ ID NO: 1705), AGGGACATCCCCAGCTCCATGCTGTGGTCG (SEQ ID NO: 259 of US20160369298; herein SEQ ID NO: 1706), AGGGACAACCCCTCCGACTCGCCCTAATCC (SEQ ID NO: 260 of US20160369298; herein SEQ ID NO: 1707), TCCTAGTAGAAGACACCCTCTCACTGCCCG (SEQ ID NO: 261 of US20160369298; herein SEQ ID NO: 1708), AGTACCATGTACACCCACTCTCCCAGTGCC (SEQ ID NO: 262 of US20160369298; herein SEQ ID NO: 1709), ATATGGACGTTCATGCTGATCACCATACCG (SEQ ID NO: 263 of US20160369298; herein SEQ ID NO: 1710), AGCAGGAGCTCCTTGGCCTCAGCGTGCGAG (SEQ ID NO: 264 of US20160369298; herein SEQ ID NO: 1711), ACAAGCAGCTTCACTATGACAACCACTGAC (SEQ ID NO: 265 of US20160369298; herein SEQ ID NO: 1712), CAGCCTAGGAACTGGCTTCCTGGACCCTGTTACCGCCAGCAGAGAGTCTCAAMAMMAV NSRVCSRSAACAACAACAGTRASTTCTCCTGGMMAGGAGCTACCAAGTACCACCTCAAT GGCAGAGACTCTCTGGTGAATCCCGGACCAGCTATGGCAAGCCACRRGGACRRCRMSRR SARSTTTTTTCCTCAGAGCGGGGTTCTCATCTTTGGGAAGSAARRCRSCRVSRVARVCRAT RYCGMSNHCRVMVRSGTCATGATTACAGACGAAGAGGAGATCTGGAC (SEQ ID NO: 266 of US20160369298; herein SEQ ID NO: 1713), TGGGACAATGGCGGTCGTCTCTCAGAGTTKTKKT (SEQ ID NO: 267 of US20160369298; herein SEQ ID NO: 1714), AGAGGACCKKTCCTCGATGGTTCATGGTGGAGTTA (SEQ ID NO: 268 of US20160369298; herein SEQ ID NO: 1715), CCACTTAGGGCCTGGTCGATACCGTTCGGTG (SEQ ID NO: 269 of US20160369298; herein SEQ ID NO: 1716), and TCTCGCCCCAAGAGTAGAAACCCTTCSTTYYG (SEQ ID NO: 270 of US20160369298; herein SEQ ID NO: 1717).

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID NO:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID NO: 1718), or AAV9 (SEQ ID NO: 11 of WO2016134375; herein SEQ ID NO: 1719). In some embodiments, modifications, such as insertions are made in AAV2 proteins at P34-A35, T138-A139, A139-P140, G453-T454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of WO2016134375; herein SEQ ID NO: 1720), or GETRAPL (SEQ ID NO: 4 of WO2016134375; herein SEQ ID NO: 1721).

In some embodiments, the AAV serotype may be modified as described in the United States Publication US 20170145405 the contents of which are herein incorporated by reference in their entirety. AAV serotypes may include, modified AAV2 (e.g., modifications at Y444F. Y500F. Y730F and/or S662V), modified AAV3 (e.g., modifications at Y705F, Y731F and/or T492V), and modified AAV6 (e.g., modifications at S663V and/or T492V).

In some embodiments, the AAV serotype may be modified as described in the International Publication WO2017083722 the contents of which are herein incorporated by reference in their entirety. AAV serotypes may include, AAV1 (Y705+731F+T492V), AAV2 (Y444+500+730F+T491V), AAV3 (Y705+731F), AAV5, AAV 5(Y436+693+719F), AAV6 (VP3 variant Y705F/Y731F/T492V), AAV8 (Y733F), AAV9, AAV9 (VP3 variant Y731F), and AAV10 (Y733F).

In some embodiments, the AAV serotype may comprise, as described in International Patent Publication WO2017015102, the contents of which are herein incorporated by reference in their entirety, an engineered epitope comprising the amino acids SPAKFA (SEQ ID NO: 24 of WO2017015102; herein SEQ ID NO: 1722) or NKDKLN (SEQ ID NO:2 of WO2017015102; herein SEQ ID NO: 1723). The epitope may be inserted in the region of amino acids 665 to 670 based on the numbering of the VP1 capsid of AAV8 (SEQ ID NO: 3 of WO2017015102) and/or residues 664 to 668 of AAV3B (SEQ ID NO: 3).

In some embodiments, the AAV serotype may be, or may have a sequence as described in International Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379,451-459, 472-473,493-500, 528-534, 547-552, 588-597, 709-710, 716-722 of AAV1, in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV. The amino acid substitution may be, but is not limited to, any of the amino acid sequences described in WO2017058892. In some embodiments, the AAV may comprise an amino acid substitution at residues 256L, 258K, 259Q, 261S, 263A, 264S, 265T, 266G, 272H, 385S, 386Q, S472R, V473D, N500E 547S, 709A, 710N, 716D, 717N, 718N, 720L, A456T, Q457T, N458Q, K459S, T492S, K493A, S586R, S587G, S588N, T589R and/or 722T of AAV1 (SEQ ID NO: 1 of WO2017058892) in any combination, 244N, 246Q, 248R, 249E, 250I, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 7055, 706T, 707G, 708E, 709Y and/or 710R of AAV5 (SEQ ID NO:5 of WO2017058892) in any combination, 248R, 316V, 317Q, 318D, 319S, 443N, 530N, 531S, 532Q 533P, 534A, 535N, 540A, 541 T, 542Y, 543L, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 5891 of AAV6 (SEQ ID NO:6 WO2017058892) in any combination, 457T, 459N, 496G, 499N, 500N, 589Q, 590N and/or 592A of AAV8 (SEQ ID NO: 8 WO2017058892) in any combination, 4511, 452N, 453G, 454S, 455G, 456Q, 457N and/or 458Q of AAV9 (SEQ ID NO: 9 WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764, the contents of which are herein incorporated by reference in their entirety. The sequences of amino acid may be, but not limited to, N-S-S, S-X-S, S-S-Y, N-X-S, N-S-Y, S-X-Y and N-X-Y, where N, X and Y are, but not limited to, independently non-serine, or non-threonine amino acids, wherein the AAV may be, but not limited to AAV1, AAV2, AAV3, AAV4, AAV5, AAV6, AAV7, AAV8, AAV9, AAV10, AAV11 and AAV12. In some embodiments, the AAV may include a deletion of at least one amino acid at positions 156, 157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

In some embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may include a peptide such as, but not limited to, PHP.B, PHP.B2, PHP.B3, PHP.A, PHP.S, G2A12, G2A15, G2A3, G2B4, and G2B5. In some embodiments, these AAV serotypes may be AAV9 (SEQ ID NO: 11 or 138) derivatives with a 7-amino acid insert between amino acids 588-589. Non-limiting examples of these 7-amino acid inserts include TLAVPFK (PHP.B; SEQ ID NO: 1262), SVSKPFL (PHP.B2; SEQ ID NO: 1270), FTLTTPK (PHP.B3; SEQ ID NO: 1271), YTLSQGW (PHP.A; SEQ ID NO: 1277), QAVRTSL (PHP.S; SEQ ID NO: 1321), LAKERLS (G2A3; SEQ ID NO: 1322), MNSTKNV (G2B4; SEQ ID NO: 1323), and/or VSGGHHS (G2B5; SEQ ID NO: 1324).

In some embodiments, the AAV serotype may be as described in Jackson et al (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (i.e., CBA or CMV).

In some embodiments, the AAV serotype is a serotype comprising the AAVPHP.N (PHP.N) peptide, or a variant thereof.

In some embodiments the AAV serotype is a serotype comprising the AAVPHP.B (PHP.B) peptide, or a variant thereof.

In some embodiments, the AAV serotype is a serotype comprising the AAVPHP.A (PHP.A) peptide, or a variant thereof.

In some embodiments, the AAV serotype is a serotype comprising the PHP.S peptide, or a variant thereof.

In some embodiments, the AAV serotype is a serotype comprising the PHP.B2 peptide, or a variant thereof.

In some embodiments, the AAV serotype is a serotype comprising the PHP.B3 peptide, or a variant thereof.

In some embodiments, the AAV serotype is a serotype comprising the G2B4 peptide, or a variant thereof.

In some embodiments, the AAV serotype is a serotype comprising the G2B5 peptide, or a variant thereof.

In some embodiments the AAV serotype is VOY101, or a variant thereof. In some embodiments, the VOY101 capsid comprises the amino acid sequence SEQ ID NO: 1. In some embodiments, the VOY101 amino acid sequence is encoded by a nucleotide sequence comprising SEQ ID NO: 2. In some embodiments, the VOY101 capsid comprises an amino acid sequence at least 70% identical to SEQ ID NO: 1, such as, 70%, 75%, 80%, 85%, 90%, 91%, 92%, 93%, 94%, 95%, 96%, 97%, 98%, 99%, or greater than 99%. In some embodiments, the VOY101 capsid comprises a nucleotide sequence at least 70% identical to SEQ ID NO: 2, such as, 70%, 75%, 80%, 85%, 90%, 91%, 92%, 93%, 94%, 95%, 96%, 97%, 98%, 99%, or greater than 99%.

In some embodiments, the AAV serotype is VOY201, or a variant thereof. In some embodiments, the VOY201 capsid comprises the amino acid sequence SEQ ID NO: 4534. In some embodiments, the VOY201 amino acid sequence is encoded by a nucleotide sequence comprising SEQ ID NO: 3. In some embodiments, the VOY201 capsid comprises an amino acid sequence at least 70% identical to SEQ ID NO: 4534, such as, 70%, 75%, 80%, 85%, 90%, 91%, 92%, 93%, 94%, 95%, 96%, 97%, 98%, 99%, or greater than 99%. In some embodiments, the VOY201 capsid comprises a nucleotide sequence at least 70% identical to SEQ ID NO: 3, such as, 70%, 75%, 80%, 85%, 90%, 91%, 92%, 93%, 94%, 95%, 96%, 97%, 98%, 99%, or greater than 99%.

In some embodiments, the AAV serotype is PHP.B, or a variant thereof. In some embodiments, the PHP.B capsid comprises the amino acid sequence SEQ ID NO: 5. In some embodiments, the PHP.B amino acid sequence is encoded by a nucleotide sequence comprising SEQ ID NO: 6. In some embodiments, the PHP.B capsid comprises an amino acid sequence at least 70% identical to SEQ ID NO: 5, such as, 70%, 75%, 80%, 85/6, 90%, 91%, 92%, 93%, 94%, 95%, 96%, 97%, 98%, 99%, or greater than 99%. In some embodiments, the PHP.B capsid comprises a nucleotide sequence at least 70% identical to SEQ ID NO: 6, such as, 70%, 75%, 80%, 85%, 90%, 91%, 92%, 93%, 94%, 95%, 96%, 97%, 98%, 99%, or greater than 99%.

In some embodiments, the AAV serotype is PHP.N, or a variant thereof. In some embodiments, the PHP.N capsid comprises the amino acid sequence SEQ ID NO: 4. In some embodiments, the PHP.N capsid comprises an amino acid sequence at least 70% identical to SEQ ID NO: 4, such as, 70%, 75%, 80%, 85%, 90%, 91%, 92%, 93%, 94%, 95%, 96%, 97%, 98%, 99%, or greater than 99%.

In some embodiments the AAV serotype is AAV9, or a variant thereof. In some embodiments, the AAV9 capsid comprises the amino acid sequence SEQ ID NO: 138. In some embodiments, the AAV9 amino acid sequence is encoded by a nucleotide sequence comprising SEQ ID NO: 137. In some embodiments, the AAV9 capsid comprises an amino acid sequence at least 70% identical to SEQ ID NO: 138, such as, 70%, 75%, 80%, 85%, 90%, 91%, 92%, 93%, 94%, 95%, 96%, 97%, 98%, 99%, or greater than 99%. In some embodiments, the AAV9 capsid comprises a nucleotide sequence at least 70% identical to SEQ ID NO: 137, such as, 70%, 75%, 80%, 85%, 90%, 91%, 92%, 93%, 94%, 95%, 96%, 97%, 98%, 99%, or greater than 99%.

In some embodiments, the AAV serotype is AAV9K449R, or a variant thereof. In some embodiments, the AAV9K449R capsid comprises the amino acid sequence SEQ ID NO: 11. In some embodiments, the AAV9K449R capsid comprises an amino acid sequence at least 70% identical to SEQ ID NO: 11, such as, 70%, 75%, 80%, 85%, 90%, 91%, 92%, 93%, 94%, 95%, 96%, 97%, 98%, 99%, or greater than 99%.

In some embodiments, the AAV capsid allows for blood brain barrier penetration following intravenous administration. Non-limiting examples of such AAV capsids include AAV9, AAV9K449R, VOY101, VOY201, or AAV capsids comprising a peptide insert such as, but not limited to, AAVPHP.N (PHP.N), AAVPHP.B (PHP.B), PHP.S, G2A3, G2B4, G2B5, G2A12, G2A15, PHP.B2, PHP.B3, or AAVPHP.A (PHP.A).

In some embodiments, the AAV capsid is suitable for intramuscular administration and/or transduction of muscle fibers. Non-limiting examples of such AAV capsids include AAV2, AAV3, AAV8 and variants thereof such as, but not limited to, AAV2 variants, AAV2/3 variants, AAV8 variants, and/or AAV2/3/8 variants.

In some embodiments, the AAV serotype is an AAV2 variant. As a non-limiting example, the AAV serotype is an AAV2 variant comprising SEQ ID NO: 2679 or a fragment or variant thereof. As a non-limiting example, the AAV serotype is at least 70% identical to SEQ ID NO: 2679, such as, 70%, 75%, 80%, 85%, 90%, 91%, 92%, 93%, 94%, 95%, 96%, 97%, 98%, 99%, or greater than 99%.

In some embodiments, the AAV serotype is an AAV2/3 variant. As a non-limiting example, the AAV serotype is an AAV2/3 variant comprising SEQ ID NO: 2809 or a fragment or variant thereof. As a non-limiting example, the AAV serotype is an AAV2/3 variant which is at least 70% identical to SEQ ID NO: 2809, such as, 70%, 75%, 80%, 85%, 90%, 91%, 92%, 93%, 94%, 95%, 96%, 97%, 98%, 99%, or greater than 99%. As a non-limiting example, the AAV serotype is an AAV2/3 variant comprising SEQ ID NO: 2871 or a fragment or variant thereof. As a non-limiting example, the AAV serotype is an AAV2/3 variant which is at least 70% identical to SEQ ID NO: 2871, such as, 70%, 75%, 80%, 85%, 90%, 91%, 92%, 93%, 94%, 95%, 96%, 97%, 98%, 99%, or greater than 99%.

In some embodiments, the AAV serotype may comprise a capsid amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described herein.

In some embodiments, the AAV serotype may be encoded by a capsid nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described herein.

In some embodiments, the AAV serotype is selected for use due to its tropism for cells of the central nervous system. In some embodiments, the cells of the central nervous system are neurons. In another embodiment, the cells of the central nervous system are astrocytes.

In some embodiments, the AAV serotype is selected for use due to its tropism for cells of the muscle(s).

In some embodiments, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Viral Genome Component: Inverted Terminal Repeals (ITRs)

The AAV particles of the present disclosure comprise a viral genome with at least one ITR region and a payload region. In some embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes may be comprised of naturally occurring polynucleotide sequences or recombinantly derived polynucleotide sequences.

The ITRs may be derived from the same serotype as the capsid, selected from any of the serotypes listed in Table 1, or a derivative thereof. The ITR may be of a different serotype than the capsid. In some embodiments, the AAV particle has more than one ITR. In a non-limiting example, the AAV particle has a viral genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments, the ITRs are 140-142 nucleotides in length. Non-limiting examples of ITR length are 102, 130, 140, 141, 142, 145 nucleotides in length, and those having at least 95% identity thereto.

In some embodiments, each ITR may be 141 nucleotides in length.

In some embodiments, each ITR may be 130 nucleotides in length.

In some embodiments, the AAV particles comprise two ITRs and one ITR is 141 nucleotides in length and the other ITR is 130 nucleotides in length.

### Viral Genome Component: Promoters

In some embodiments, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides in a target cell may require a specific promoter, including but not limited to, a promoter that is species specific, inducible, tissue-specific, or cell cycle-specific (Parr et al., Nat. Med 3:1145-9 (1997); the contents of which are herein incorporated by reference in their entirety).

In some embodiments, the promoter is deemed to be efficient when it drives expression of the polypeptide(s) encoded in the payload region of the viral genome of the AAV particle.

In some embodiments, the promoter is a promoter deemed to be efficient when it drives expression in the cell being targeted.

In some embodiments, the promoter drives expression of the polypeptides (e.g., a functional antibody) for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiments, the promoter drives expression of the polypeptides (e.g., a functional antibody) for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 1 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters. B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US20110212529, the contents of which are herein incorporated by reference in their entirety)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800, or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350,360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In some embodiments, the promoter is a combination of a 382 nucleotide CMV-enhancer sequence and a 260 nucleotide CBA-promoter sequence.

In some embodiments, the viral genome comprises a ubiquitous promoter. Non-limiting examples of ubiquitous promoters include CMV, CBA (including derivatives CAG, CB6, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EF1α, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EF1α promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HOH construct with a hGUSB promoter, an HSV-1LAT promoter and an NSE promoter and found that the HOH construct showed weaker expression than NSE in mouse brain. Passini and Wolfe (J. Virol. 2001, 12382-12392, the contents of which are herein incorporated by reference in its entirety) evaluated the long-term effects of the HOH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920-nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus, and hypothalamus (See e.g., Drews et al. Identification of evolutionary conserved, functional noncoding elements in the promoter region of the sodium channel gene SCN8A. Mamm Genome (2007) 18:723-731; and Raymond et al. Expression of Alternatively Spliced Sodium Channel α-subunit genes. Journal of Biological Chemistry (2004) 279(44) 46234-46241; the contents of each of which are herein incorporated by reference in their entireties).

Any of promoters taught by the aforementioned Yu, Soderblom, Gill, Husain. Passini, Xu, Drews, or Raymond may be used in the present disclosures.

In some embodiments, the promoter is not cell specific.

In some embodiments, the promoter is a ubiquitin c (UBC) promoter. The UBC promoter may have a size of 300-350 nucleotides. As a non-limiting example, the UBC promoter is 332 nucleotides.

In some embodiments, the promoter is a β-glucuronidase (GUSB) promoter. The GUSB promoter may have a size of 350-400 nucleotides. As a non-limiting example, the GUSB promoter is 378 nucleotides.

In some embodiments, the promoter is a neurofilament light (NFL) promoter. The NFL promoter may have a size of 600-700 nucleotides. As a non-limiting example, the NFL promoter is 650 nucleotides.

In some embodiments, the promoter is a neurofilament heavy (NFH) promoter. The NFH promoter may have a size of 900-950 nucleotides. As a non-limiting example, the NFH promoter is 920 nucleotides.

In some embodiments, the promoter is a scn8a promoter. The scn8a promoter may have a size of 450-500 nucleotides. As a non-limiting example, the scn8a promoter is 470 nucleotides.

In some embodiments, the promoter is a phosphoglycerate kinase 1 (PGK) promoter.

In some embodiments, the promoter is a chicken β-actin (CBA) promoter, or a variant thereof.

In some embodiments, the promoter is a CB6 promoter.

In some embodiments, the promoter is a minimal CB promoter.

In some embodiments, the promoter is a cytomegalovirus (CMV) promoter.

In some embodiments, the promoter is a CAG promoter.

In some embodiments, the promoter is a GFAP promoter.

In some embodiments, the promoter is a synapsin promoter.

In some embodiments, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic CS-12.

In some embodiments, the promoter is an RNA pol III promoter. As a non-limiting example, the RNA pol III promoter is U6. As a non-limiting example, the RNA pol III promoter is H1.

In some embodiments, the viral genome comprises two promoters. As a non-limiting example, the promoters are an EF1α promoter and a CMV promoter.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter; and (9) GFAP promoter.

In some embodiments, the viral genome comprises an engineered promoter.

In another embodiment, the viral genome comprises a promoter from a naturally expressed protein.

### Viral Genome Component: Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles to enhance expression in hepatic cell lines or liver.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

In some embodiments, the Kozak sequence is GAGGAGCCACC (SEQ ID NO: 4543).

In some embodiments, the Kozak sequence is GCCGCCACCATG (SEQ ID NO: 2114)

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-a, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

In some embodiments, the viral genome may include at least one miRNA seed, binding site or full sequence, microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In some embodiments, the viral genome may be engineered to include, alter or remove at least one miRNA binding site, sequence, or seed region.

Any UTR from any gene known in the art may be incorporated into the viral genome of the AAV particle. These UTRs, or portions thereof, may be placed in the same orientation as in the gene from which they were selected, or they may be altered in orientation or location. In some embodiments, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTRs which is not a variant of a wild type UTR.

In some embodiments, the viral genome of the AAV particle comprises UTRs which have been selected from a family of transcripts whose proteins share a common function, structure, feature or property.

### Viral Genome Component: Polyadenylation Sequence

In some embodiments, the viral genome of the AAV particles of the present disclosure comprise at least one polyadenylation sequence. The viral genome of the AAV particle may comprise a polyadenylation sequence between the 3′ end of the payload coding sequence and the 5′ end of the 3′ITR.

In some embodiments, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides in length.

In some embodiments, the polyadenylation sequence is 50-100 nucleotides in length.

In some embodiments, the polyadenylation sequence is 50-150 nucleotides in length.

In some embodiments, the polyadenylation sequence is 50-160 nucleotides in length.

In some embodiments, the polyadenylation sequence is 50-200 nucleotides in length.

In some embodiments, the polyadenylation sequence is 60-100 nucleotides in length.

In some embodiments, the polyadenylation sequence is 60-150 nucleotides in length.

In some embodiments, the polyadenylation sequence is 60-160 nucleotides in length.

In some embodiments, the polyadenylation sequence is 60-200 nucleotides in length.

In some embodiments, the polyadenylation sequence is 70-100 nucleotides in length.

In some embodiments, the polyadenylation sequence is 70-150 nucleotides in length.

In some embodiments, the polyadenylation sequence is 70-160 nucleotides in length.

In some embodiments, the polyadenylation sequence is 70-200 nucleotides in length.

In some embodiments, the polyadenylation sequence is 80-100 nucleotides in length.

In some embodiments, the polyadenylation sequence is 80-150 nucleotides in length.

In some embodiments, the polyadenylation sequence is 80-160 nucleotides in length.

In some embodiments, the polyadenylation sequence is 80-200 nucleotides in length.

In some embodiments, the polyadenylation sequence is 90-100 nucleotides in length.

In some embodiments, the polyadenylation sequence is 90-150 nucleotides in length.

In some embodiments, the polyadenylation sequence is 90-160 nucleotides in length.

In some embodiments, the polyadenylation sequence is 90-200 nucleotides in length.

In some embodiments, the polyadenylation sequence is 127 nucleotides in length.

In some embodiments, the polyadenylation sequence is 477 nucleotides in length.

In some embodiments, the polyadenylation sequence is 552 nucleotides in length.

### Viral Genome Component: Linkers

Viral genomes may be engineered with one or more spacer or linker regions to separate coding or non-coding regions.

In some embodiments, the payload region of the AAV particle may optionally encode one or more linker sequences. In some cases, the linker may be a peptide linker that may be used to connect the polypeptides encoded by the payload region (i.e., light and heavy antibody chains during expression). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome. Non-limiting examples of linkers that may be encoded by the payload region of an AAV particle viral genome are given in Table 2.

TABLE 2
Linkers

| Linker ID | Description | Length | SEQ ID NO |
|---|---|---|---|
| Linker1 | Furin | 12 | 1724 |
| Linker2 | Furin | 12 | 1725 |
| Linker3 | T2A | 54 | 1726 |
| Linker4 | F2A | 75 | 1727 |
| Linker5 | P2A | 66 | 1728 |
| Linker6 | SG4S (SEQ ID NO: 4536) | 18 | 1729 |
| Linker7 | (G4S)3 (SEQ ID NO: 4537) | 45 | 1730 |
| Linker8 | (G4S)5 (SEQ ID NO: 4538) | 75 | 1731 |
| Linker9 | IRES | 609 | 1732 |
| Linker10 | IRES-2 | 623 | 1733 |
| Linker11 | hIgG2 hinge | 54 | 1734 |
| Linker12 | hIgG3 hinge | 108 | 1735 |
| Linker13 | hIgG3-2 hinge | 153 | 1736 |
| Linker14 | hIgG3-3 hinge | 198 | 1737 |
| Linker15 | msiGG-1 hinge | 45 | 1738 |
| Linker16 | msiGG1 hinge | 18 | 1739 |
| Linker17 | HigG3 hinge | 198 | 2244 |
| Linker18 | G4S (SEQ ID NO: 4535) | 15 | 2245 |
| Linker19 | (G4S)2 (SEQ ID NO: 4539) | 30 | 2246 |
| Linker20 | (G4S)3 (SEQ ID NO: 4537) | 45 | 2247 |
| Linker21 | (G4S)4 (SEQ ID NO: 4540) | 60 | 2248 |
| Linker22 | (G4S)5 (SEQ ID NO: 4538) | 75 | 2249 |
| Linker23 | (G4S)5 (SEQ ID NO: 4538) | 75 | 2250 |
| Linker24 | (G4S)6 (SEQ ID NO: 4541) | 90 | 2251 |
| Linker25 | (G4S)8 (SEQ ID NO: 4542) | 120 | 2252 |
| Linker26 | (G4S)8 (SEQ ID NO: 4542) | 120 | 2253 |
| Linker27 | (G4S)4 (SEQ ID NO: 4540) | 60 | 2254 |
| Linker28 | (G4S)6 (SEQ ID NO: 4541) | 90 | 2259 |

Some payload regions encode linkers comprising furin cleavage sites. Furin is a calcium dependent serine endoprotease that cleaves proteins just downstream of a basic amino acid target sequence (Arg-X-(Arg/Lys)-Arg) (Thomas, G., 2002. Nature Reviews Molecular Cell Biology 3(10): 753-66; the contents of which are herein incorporated by reference in its entirety). Furin is enriched in the trans-golgi network where it is involved in processing cellular precursor proteins. Furin also plays a role in activating a number of pathogens. This activity can be taken advantage of for expression of polypeptides.

2A peptides are small “self-cleaving” peptides (18-22 amino acids) derived from viruses such as foot-and-mouth disease virus (F2A), porcine teschovirus-1 (P2A), Thoseaasigna virus (T2A), or equine rhinitis A virus (E2A). The 2A designation refers specifically to a region of picornavirus polyproteins that lead to a ribosomal skip at the glycyl-prolyl bond in the C-terminus of the 2A peptide (Kim, J. H. et al., 2011. PLoS One 6(4): e18556; the contents of which are herein incorporated by reference in its entirety). This skip results in a cleavage between the 2A peptide and its immediate downstream peptide. As opposed to IRES linkers, 2A peptides generate stoichiometric expression of proteins flanking the 2A peptide and their shorter length can be advantageous in generating viral expression vectors.

Internal ribosomal entry site (IRES) is a nucleotide sequence (>500 nucleotides) that allows for initiation of translation in the middle of an mRNA sequence (Kim, J. H. et al., 2011. PLoS One 6(4): e18556; the contents of which are herein incorporated by reference in its entirety). Use of an IRES sequence ensures co-expression of genes before and after the IRES, though the sequence following the IRES may be transcribed and translated at lower levels than the sequence preceding the IRES sequence.

In some embodiments, the payload region may encode one or more linkers comprising cathepsin, matrix metalloproteinases or legumain cleavage sites. Such linkers are described e.g. by Cizeau and Macdonald in International Publication No. WO2008052322, the contents of which are herein incorporated in their entirety. Cathepsins are a family of proteases with unique mechanisms to cleave specific proteins. Cathepsin B is a cysteine protease and cathepsin D is an aspartyl protease. Matrix metalloproteinases are a family of calcium-dependent and zinc-containing endopeptidases. Legumain is an enzyme catalyzing the hydrolysis of (-Asn-Xaa-) bonds of proteins and small molecule substrates.

In some embodiments, payload regions may encode linkers that are not cleaved. Such linkers may include a simple amino acid sequence, such as a glycine rich sequence. In some cases, linkers may comprise flexible peptide linkers comprising glycine and serine residues. The linker may comprise flexible peptide linkers of different lengths, e.g. nxG4S, where n=1-10 (SEQ ID NO: 4544), and the length of the encoded linker varies between 5 and 50 amino acids. In a non-limiting example, the linker may be 5xG4S (SEQ ID NO: 4544). These flexible linkers are small and without side chains so they tend not to influence secondary protein structure while providing a flexible linker between antibody segments (George, R. A., et al., 2002. Protein Engineering 15(11): 871-9; Huston, J. S. et al., 1988. PNAS 85:5879-83; and Shan, D. et al., 1999. Journal of Immunology. 162(11):6589-95; the contents of each of which are herein incorporated by reference in their entirety). Furthermore, the polarity of the serine residues improves solubility and prevents aggregation problems.

In some embodiments, payload regions may encode small and unbranched serine-rich peptide linkers, such as those described by Huston et al. in U.S. Pat. No. 5,525,491, the contents of which are herein incorporated in their entirety. Polypeptides encoded by the payload region, linked by serine-rich linkers, have increased solubility.

In some embodiments, payload regions may encode artificial linkers, such as those described by Whitlow and Filpula in U.S. Pat. No. 5,856,456 and Ladner et al. in U.S. Pat. No. 4,946,778, the contents of each of which are herein incorporated by their entirety.

In some embodiments, the payload region encodes at least one G4S3 linker (“G4S3” disclosed as SEQ ID NO: 4537).

In some embodiments, the payload region encodes at least one G4S linker (“G4S” disclosed as SEQ ID NO: 4535).

In some embodiments, the payload region encodes at least one furin site.

In some embodiments, the payload region encodes at least one T2A linker.

In some embodiments, the payload region encodes at least one F2A linker.

In some embodiments, the payload region encodes at least one P2A linker.

In some embodiments, the payload region encodes at least one IRES sequence.

In some embodiments, the payload region encodes at least one G4S5 linker (“G4S5” disclosed as SEQ ID NO: 4538).

In some embodiments, the payload region encodes at least one furin and one 2A linker. As non-limiting examples, the payload region may comprise furin and T2A linkers or furin and F2A linkers.

In some embodiments, the payload region encodes at least one hinge region. As a non-limiting example, the hinge is an IgG hinge.

In some embodiments, the linker region may be 1-50, 1-100, 50-100, 50-150, 100-150, 100-200, 150-200, 150-250, 200-250, 200-300, 250-300, 250-350, 300-350, 300-400, 350-400, 350-450, 400-450, 400-500, 450-500, 450-550, 500-550, 500-600, 550-600, 550-650, or 600-650 nucleotides in length. The linker region may have a length of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140, 145, 150, 151, 152, 153, 154, 155, 156, 157, 158, 159, 160, 165, 170, 171, 172, 173, 174, 175, 176, 177, 178, 179, 180, 185, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 640, 650 or greater than 650. In some embodiments, the linker region may be 12 nucleotides in length. In some embodiments, the linker region may be 15 nucleotides in length, In some embodiments, the linker region may be 18 nucleotides in length. In some embodiments, the linker region may be 30 nucleotides in length. In some embodiments, the linker region may be 45 nucleotides in length. In some embodiments, the linker region may be 54 nucleotides in length. In some embodiments, the linker region may be 60 nucleotides in length. In some embodiments, the linker region may be 66 nucleotides in length. In some embodiments, the linker region may be 75 nucleotides in length. In some embodiments, the linker region may be 78 nucleotides in length. In some embodiments, the linker region may be 87 nucleotides in length. In some embodiments, the linker region may be 108 nucleotides in length. In some embodiments, the linker region may be 120 nucleotides in length. In some embodiments, the linker region may be 153 nucleotides in length. In some embodiments, the linker region may be 198 nucleotides in length. In some embodiments, the linker region may be 609 nucleotides in length. In some embodiments, the linker region may be 623 nucleotides in length.

### Viral Genome Component: Introns

In some embodiments, the payload region comprises at least one element to enhance the expression such as one or more introns or portions thereof. Non-limiting examples of introns include, MVM (67-97 bps), F.IX truncated intron 1 (300 bps), 0-globin SD/immunoglobulin heavy chain splice acceptor (250 bps), adenovirus splice donor/immunoglobin splice acceptor (500 bps). SV40 late splice donor/splice acceptor (19S/16S) (180 bps) and hybrid adenovirus splice donor/IgG splice acceptor (230 bps).

In some embodiments, the intron or intron portion may be 1-100, 100-500, 500-1000, or 1000-1500 nucleotides in length. The intron may have a length of 10, 15, 20, 30, 40, 50, 60, 70, 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or greater than 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500. In some embodiments, the intron may be 15 nucleotides in length. In some embodiments, the intron may be 32 nucleotides in length. In some embodiments, the intron may be 41 nucleotides in length. In some embodiments, the intron may be 53 nucleotides in length. In some embodiments, the intron may be 54 nucleotides in length. In some embodiments, the intron may be 59 nucleotides in length. In some embodiments, the intron may be 73 nucleotides in length. In some embodiments, the intron may be 102 nucleotides in length. In some embodiments, the intron may be 134 nucleotides in length. In some embodiments, the intron may be 168 nucleotides in length. In some embodiments, the intron may be 172 nucleotides in length. In some embodiments, the intron may be 292 nucleotides in length. In some embodiments, the intron may be 347 nucleotides in length. In some embodiments, the intron may be 387 nucleotides in length. In some embodiments, the intron may be 491 nucleotides in length. In some embodiments, the intron may be 566 nucleotides in length. In some embodiments, the intron may be 1074 nucleotides in length.

Any, or all components of a viral genome may be modified or optimized to improve expression or targeting of the payload. Such components include, but are not limited to, intron, signal peptide sequences, antibody heavy chain and/or light chain 5′ to 3′ order, antibody heavy chain and/or light chain codons, linkers, cleavage sites, polyadenylation sequences, stuffer sequences, other regulatory sequences, and/or the backbone of the ITR to ITR sequence.

### Payloads

The AAV particles of the present disclosure comprise at least one payload region. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. Payloads of the present disclosure typically encode polypeptides (e.g., antibodies or antibody-based compositions) or fragments or variants thereof.

The payload region may be constructed in such a way as to reflect a region similar to or mirroring the natural organization of an mRNA.

The payload region may comprise a combination of coding and non-coding nucleic acid sequences.

In some embodiments, the AAV payload region may encode a coding or non-coding RNA.

In some embodiments, the AAV particle comprises a viral genome with a payload region comprising nucleic acid sequences encoding more than one polypeptide of interest (e.g., an antibody). In such an embodiment, a viral genome encoding more than one polypeptide may be replicated and packaged into a viral particle. A target cell transduced with a viral particle comprising more than one polypeptide may express each of the polypeptides in a single cell.

In some embodiments, an AAV particle comprises a viral genome with a payload region comprising a nucleic acid sequence encoding a heavy chain and a light chain of an antibody, or fragments thereof. The heavy chain and light chain are expressed and assembled to form the antibody which is secreted.

In some embodiments, the payload region may comprise at least one inverted terminal repeat (ITR), a promoter region, an intron region, and a coding region. In some embodiments, the coding region comprises a heavy chain region and/or a light chain region of an antibody, or a fragment thereof, and any two components may be separated by a linker region.

In some embodiments, the coding region may comprise a payload region with a heavy chain and light chain sequence separated by a linker and/or a cleavage site. In some embodiments, the heavy and light chain sequence is separated by an IRES sequence. In some embodiments, the heavy and light chain sequence is separated by a foot and mouth virus sequence. In some embodiments, the heavy and light chain sequence is separated by a foot and mouth virus sequence and a furin cleavage site. In some embodiments, the heavy and light chain sequence is separated by a porcine teschovirus-1 virus sequence. In some embodiments, the heavy and light chain sequence is separated by a porcine teschovirus-1 virus and a furin cleavage site. In some embodiments, the heavy and light chain sequence is separated by a 5xG4S sequence (“5xG4S” disclosed as SEQ ID NO: 4538).

Where the AAV particle payload region encodes a polypeptide, the polypeptide may be a peptide or protein. A protein encoded by the AAV particle payload region may comprise an antibody, an antibody related composition, a secreted protein, an intracellular protein, an extracellular protein, and/or a membrane protein. The encoded proteins may be structural or functional. In addition to the antibodies or antibody-based composition, proteins encoded by the payload region may include, in combination, certain mammalian proteins involved in immune system regulation. The AAV viral genomes encoding polypeptides described herein may be useful in the fields of human disease, viruses, infections, veterinary applications and a variety of in vivo and in vitro settings.

In some embodiments, the AAV particles are useful in the field of medicine for the treatment, prophylaxis, palliation, or amelioration of neurological diseases and/or disorders.

### Antibodies and Antibody-Based Compositions

Payload regions of the AAV particles may encode polypeptides that form one or more functional antibodies or antibody-based compositions. As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.).

As used herein, “antibody-based” or “antibody-derived” compositions are monomeric or multi-meric polypeptides which comprise at least one amino-acid region derived from a known or parental antibody sequence and at least one amino acid region derived from a non-antibody sequence, e.g., mammalian protein.

Payload regions may encode polypeptides that form or function as any antibody, including antibodies that are known in the art and/or antibodies that are commercially available. The encoded antibodies may be therapeutic, diagnostic, or for research purposes. Further, polypeptides may include fragments of such antibodies or antibodies that have been developed to comprise one or more of such fragments (e.g., variable domains or complementarity determining regions (CDRs)).

In some embodiments, the viral genome of the AAV particles may comprise nucleic acids which have been engineered to enable expression of antibodies, antibody fragments, or components of any of those described in U.S. Pat. No. 7,041,807 related to YYX epitope; US20090175884, US20110305630, US20130330275 related to misfolded proteins in cancer; US20040175775 related to PrP in eye fluid; US20030114360 related to copolymers and methods of treating prion-related diseases; WO2009121176 related to insulin-induced gene peptide compositions; US20030022243, WO2003000853 related to protein aggregation assays; WO200078344 related to prion protein peptides and uses thereof. Each of these publications are incorporated by reference in their entireties.

### Antibody Generation

In some embodiments, viral genomes of the AAV particles may encode antibodies or antibody-based compositions produced using methods known in the art. Such methods may include, but are not limited to, immunization and display technologies (e.g., phage display, yeast display, and ribosomal display). Antibodies may be developed, for example, using any naturally occurring or synthetic antigen. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. As used herein, “antigens” also refer to binding partners for specific antibodies or binding agents in a display library.

In some embodiments, the sequences of the polypeptides to be encoded in the viral genomes may be derived from antibodies produced using hybridoma technology. Host animals (e.g. mice, rabbits, goats, and llamas) may be immunized by an injection with an antigenic protein to elicit lymphocytes that specifically bind to the antigen. Lymphocytes may be collected and fused with immortalized cell lines to generate hybridomas which can be cultured in a suitable culture medium to promote growth. The antibodies produced by the cultured hybridomas may be subjected to analysis to determine binding specificity of the antibodies for the target antigen. Once antibodies with desirable characteristics are identified, corresponding hybridomas may be subcloned through limiting dilution procedures and grown by standard methods. The antibodies produced by these cells may be isolated and purified using standard immunoglobulin purification procedures.

In some embodiments, the sequences of the polypeptides to be encoded in the viral genomes may be produced using heavy and light chain variable region cDNA sequences selected from hybridomas or from other sources. Sequences encoding antibody variable domains expressed by hybridomas may be determined by extracting RNA molecules from antibody-producing hybridoma cells and producing cDNA by reverse transcriptase polymerase chain reaction (PCR). PCR may be used to amplify cDNA using primers specific for heavy and light chain sequences. PCR products may then be subcloned into plasmids for sequence analysis. Antibodies may be produced by insertion of resulting variable domain sequences into expression vectors.

In some embodiments, the sequences of the polypeptides to be encoded in the viral genomes may be generated using display technologies. Display technologies used to generate polypeptides may include any of the display techniques (e.g. display library screening techniques) disclosed in International Patent Application No. WO2014074532, the contents of which are herein incorporated by reference in their entirety. In some embodiments, synthetic antibodies may be designed, selected, or optimized by screening target antigens using display technologies (e.g. phage display technologies). Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker. In some cases, scFvs may contain the same sequence with the exception of unique sequences encoding variable loops of the CDRs. In some cases, scFvs are expressed as fusion proteins, linked to viral coat proteins (e.g. the N-terminus of the viral pIII coat protein). V_{L} chains may be expressed separately for assembly with V_{H} chains in the periplasm prior to complex incorporation into viral coats. Precipitated library members may be sequenced from the bound phage to obtain cDNA encoding desired scFvs. Antibody variable domains or CDRs from such sequences may be directly incorporated into antibody sequences for recombinant antibody production or mutated and utilized for further optimization through in vitro affinity maturation.

In some embodiments, the sequences of the polypeptides to be encoded in the viral genomes may be produced using yeast surface display technology, wherein antibody variable domain sequences may be expressed on the cell surface of Saccharomyces cerevisiae. Recombinant antibodies may be developed by displaying the antibody fragment of interest as a fusion to e.g. Aga2p protein on the surface of the yeast, where the protein interacts with proteins and small molecules in a solution, scFvs with affinity toward desired receptors may be isolated from the yeast surface using magnetic separation and flow cytometry. Several cycles of yeast surface display and isolation may be done to attain scFvs with desired properties through directed evolution.

In some embodiments, the sequence of the polypeptides to be encoded in the viral genomes (e.g., antibodies) may be designed by VERSITOPE™ Antibody Generation and other methods used by BIOATLA® and described in United States Patent Publication No. US20130281303, the contents of which are herein incorporated by reference in their entirety. In brief, recombinant monoclonal antibodies are derived from B-cells of a host immuno-challenged with one or more target antigens. These methods of antibody generation do not rely on immortalized cell lines, such as hybridoma, thereby avoiding some of the associated challenges i.e., genetic instability and low production capacity, producing high affinity and high diversity recombinant monoclonal antibodies. In some embodiments, the method is a natural diversity approach. In another embodiment, the method is a high diversity approach.

In some embodiments, the sequences of the polypeptides to be encoded in the viral genomes may be generated using the BIOATLA® natural diversity approach. In the natural diversity approach of generating recombinant monoclonal antibodies described in United States Patent Publication No. US20130281303, the original pairings of variable heavy (V_{H}) and variable light (V_{L}) domains are retained from the host, yielding recombinant monoclonal antibodies that are naturally paired. These may be advantageous due to a higher likelihood of functionality as compared to non-natural pairings of V_{H} and V_{L}. To produce the recombinant monoclonal antibodies, first a non-human host (i.e., rabbit, mouse, hamster, guinea pig, camel or goat) is immuno-challenged with an antigen of interest. In some embodiments, the host may be a previously challenged human patient. In other embodiments, the host may not have been immuno-challenged. B-cells are harvested from the host and screened by fluorescence activated cell sorting (FACS), or other method, to create a library of B-cells enriched in B-cells capable of binding the target antigen. The cDNA obtained from the mRNA of a single B-cell is then amplified to generate an immunoglobulin library of V_{H} and V_{L} domains. This library of immunoglobulins is then cloned into expression vectors capable of expressing the V_{H} and V_{L} domains, wherein the V_{H} and V_{L} domains remain naturally paired. The library of expression vectors is then used in an expression system to express the V_{H} and V_{L} domains in order to create an antibody library. Screening of the antibody library yields antibodies able to bind the target antigen, and these antibodies can be further characterized. Characterization may include one or more of the following: isoelectric point, thermal stability, sedimentation rate, folding rate, neutralization or antigen activity, antagonist or agonistic activity, expression level, specific and non-specific binding, inhibition of enzymatic activity, rigidity/flexibility, shape, charge, stability across pH, in solvents, under UV radiation, in mechanical stress conditions, or in sonic conditions, half-life, and glycosylation.

In some embodiments, the sequences of the polypeptides to be encoded in the viral genomes may be generated using the BIOATLA® high diversity approach. In the high diversity approach of generating recombinant monoclonal antibodies described in United States Patent Publication No. US20130281303, additional pairings of variable heavy (V_{H}) and variable light (V_{L}) domains are attained. To produce the recombinant monoclonal antibodies, B-cells harvested from the host are screened by fluorescence activated cell sorting (FACS), panning, or other method, to create a library of B-cells enriched in B-cells capable of binding the target antigen. The cDNA obtained from the mRNA of the pooled B-cells is then amplified to generate an immunoglobulin library of V_{H} and V_{L} domains. This library of immunoglobulins is then used in a biological display system (mammalian, yeast or bacterial cell surface display systems) to generate a population of cells displaying antibodies, fragments or derivatives comprising the V_{H} and V_{L} domains wherein, the antibodies, fragments or derivatives comprise V_{H} and V_{L} domain combinations that were not present in the B-cells in vivo. Screening of the cell population by FACS, with the target antigen, yields a subset of cells capable of binding the target antigen and the antibodies displayed on these cells can be further characterized. In an alternate embodiment of the high diversity approach, the immunoglobulin library comprises only V_{H} domains obtained from the B-cells of the immuno-challenged host, while the V_{L} domain(s) are obtained from another source.

In some embodiments, the sequences of the polypeptides to be encoded in the viral genomes may be evolved using BIOATLA® comprehensive approaches. The methods of generating recombinant monoclonal antibodies as described in United States Patent Publication No. US20130281303, further comprises evolving the recombinant antibody by comprehensive positional evolution (CPE™), CPE™ followed by comprehensive protein synthesis (CPS™), PCR shuffling, or other method.

In some embodiments, the sequence of the polypeptides to be encoded in the viral genomes (e.g., antibodies) may be derived from any of the BIOATLA® protein evolution methods described in International Publication WO2012009026, the contents of which are herein incorporated by reference in their entirety. In this method, mutations are systematically performed throughout the polypeptide or molecule of interest, a map is created providing useful informatics to guide the subsequent evolutionary steps. Not wishing to be bound by theory, these evolutionary methods typically start with a template polypeptide and a mutant is derived therefrom, which has desirable properties or characteristics. Non-limiting examples of evolutionary techniques include polymerase chain reaction (PCR), error prone PCR, oligonucleotide-directed mutagenesis, cassette mutagenesis, shuffling, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof.

In some embodiments, the BIOATLA® evolution method is Comprehensive Positional Evolution (CPE™). In CPE, naturally occurring amino acid variants are generated for each of the codons of the template polypeptide, wherein 63 different codon options exist for each amino acid variant. A set of polypeptides with single amino acid mutations are generated and the mutations are then confirmed by sequencing or other method known in the art and each amino acid change screened for improved function, neutral mutations, inhibitory mutations, expression, and compatibility with the host system. An EvoMap™ is created that describes in detail the effects of each amino acid mutation on the properties and characteristics of that polypeptide. The data from the EvoMap™ may be utilized to produce polypeptides with more than one amino acid mutation, wherein the resultant multi-site mutant polypeptides can be screened for desirable characteristics.

In some embodiments, the BIOATLA® evolution method is Synergy Evolution, wherein an EvoMap™ is used to identify amino acid positions to introduce 2-20 mutations simultaneously to produce a combinatorial effect. The resulting multi-site mutant polypeptides may be screened on one or more pre-determined characteristics to identify “upmutants” wherein the function of the mutant is improved as compared to the parent polypeptide. In some embodiments, Synergy Evolution is used to enhance binding affinity of an antibody.

In some embodiments, the BIOATLA® evolution method is Flex Evolution, wherein an EvoMap™ is used to identify fully mutable sites within a polypeptide that may then be targeted for alteration, such as introduction of glycosylation sites or chemical conjugation.

In some embodiments, the BIOATLA® evolution method is Comprehensive Positional Insertion Evolution (CPI™), wherein an amino acid is inserted after each amino acid of a template polypeptide to generate a set of lengthened polypeptides. CPI may be used to insert 1, 2, 3, 4, or 5 amino acids at each new position. The resultant lengthened polypeptides are sequenced and assayed for one or more pre-determined properties and evaluated in comparison to its template or parent molecule. In some embodiments, the binding affinity and immunogenicity of the resultant polypeptides are assayed. In some embodiments, the lengthened polypeptides are further mutated and mapped to identify polypeptides with desirable characteristics.

In some embodiments, the BIOATLA® evolution approach is Comprehensive Positional Deletion Evolution (CPD™), wherein each amino acid of the template polypeptide is individually and systematically deleted one at a time. The resultant shortened polypeptides are then sequenced and evaluated by assay for at least one pre-determined feature. In some embodiments, the shortened polypeptides are further mutated and mapped to identify polypeptides with desirable characteristics.

In some embodiments, the BIOATLA® evolution approach is Combinatorial Protein Synthesis (CPS™), wherein mutants identified in CPE, CPI, CPD, or other evolutionary techniques are combined for polypeptide synthesis. These combined mutant polypeptides are then screened for enhanced properties and characteristics. In some embodiments CPS is combined with any of the aforementioned evolutionary or polypeptide synthesis methods.

In some embodiments, the sequence of the polypeptides to be encoded in the viral genomes (e.g., antibodies) may be derived from the BIOATLA®, Comprehensive Integrated Antibody Optimization (CIAO!™) described in U.S. Pat. No. 8,859,467, the contents of which are herein incorporated by reference in their entirety. The CIAO!™ method allows for simultaneous evolution of polypeptide performance and expression optimization, within a eukaryotic cell host (i.e., mammalian or yeast cell host). First, an antibody library is generated in a mammalian cell production host by antibody cell surface display, wherein the generated antibody library targets a particular antigen of interest. The antibody library is then screened by any method known in the art, for one or more properties or characteristics. One or more antibodies of the library, with desirable properties or characteristics are chosen for further polypeptide evolution by any of the methods known in the art, to produce a library of mutant antibodies by antibody cell surface display in a mammalian cell production host. The generated mutant antibodies are screened for one or more predetermined properties or characteristics, whereby an upmutant is selected, wherein the upmutant has enhanced or improved characteristics as compared to the parent template polypeptide.

In some embodiments, the sequences of the polypeptides to be encoded in the viral genomes may be humanized by the methods of BIOATLA® as described in United States Patent Publication US20130303399, the contents of which are herein incorporated by reference in their entirety. In this method, for generating enhanced full-length humanized antibodies in mammalian cells, no back-mutations are required to retain affinity to the antigen and no CDR grafting or phage-display is necessary. The generated humanized antibody has reduced immunogenicity and equal or greater affinity for the target antigen as compared to the parent antibody. The variable regions or CDRs of the generated humanized antibody are derived from the parent or template, whereas the framework and constant regions are derived from one or more human antibodies. To start, the parent, or template antibody is selected, cloned and each CDR sequence identified and synthesized into a CDR fragment library. Double stranded DNA fragment libraries for V_{H} and V_{L} are synthesized from the CDR fragment encoding libraries, wherein at least one CDR fragment library is derived from the template antibody and framework (FW) fragment encoding libraries, wherein the FW fragment library is derived from a pool of human frameworks obtained from natively expressed and functional human antibodies. Stepwise liquid phase ligation of FW and CDR encoding fragments is then used to generate both V_{H} and V_{L} fragment libraries. The V_{H} and V_{L} fragment libraries are then cloned into expression vectors to create a humanization library, which is further transfected into cells for expression of full length humanized antibodies and used to create a humanized antibody library. The humanized antibody library is then screened to determine expression level of the humanized antibodies, affinity or binding ability for the antigen, and additional improved or enhanced characteristics, as compared to the template or parent antibody. Non-limiting examples of characteristics that may be screened include equilibrium dissociation constant (K_{D}), stability, melting temperature (Tₘ), pI, solubility, expression level, reduced immunogenicity, and improved effector function.

In some embodiments, the sequences of the polypeptides to be encoded in the viral genomes may be generated by the BIOATLA® method for preparing conditionally active antibodies as described in International Publications WO2016033331 and WO2016036916, the contents of which are herein incorporated by reference in their entirety. As used herein, the term “conditionally active” refers to a molecule that is active at an aberrant condition. Further, the conditionally active molecule may be virtually inactive at normal physiological conditions. Aberrant conditions may result from changes in pH, temperature, osmotic pressure, osmolality, oxidative stress, electrolyte concentration, and/or chemical or proteolytic resistance, as non-limiting examples.

The method of preparing a conditionally active antibody is described in International Publications WO2016033331 and WO2016036916 and summarized herein. Briefly, a wild-type polypeptide is selected and the DNA is evolved to create mutant DNAs. Non-limiting examples of evolutionary techniques that may be used to evolve the DNA include polymerase chain reaction (PCR), error prone PCR, shuffling, oligonucleotide-directed mutagenesis, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof. Once mutant DNAs are created, they are expressed in a eukaryotic cell production host (i.e., fungal, insect, mammalian, adenoviral, plant), wherein a mutant polypeptide is produced. The mutant polypeptide and the corresponding wild-type polypeptide are then subjected to assays under both normal physiological conditions and aberrant conditions in order to identify mutants that exhibit a decrease in activity in the assay at normal physiological conditions as compared to the wild-type polypeptide and/or an increase in activity in the assay under aberrant conditions, as compared to the corresponding wild-type polypeptide. The desired conditionally active mutant may then be produced in the aforementioned eukaryotic cell production host.

In some embodiments, the conditionally active antibody is a “mirac protein” as described by BIOATLA® in U.S. Pat. No. 8,709,755, the contents of which are herein incorporated by reference in their entirety. As used herein “mirac protein” refers to a conditionally active antibody that is virtually inactive at body temperature but active at lower temperatures.

In some embodiments, the sequence of the polypeptides to be encoded in the viral genomes (e.g., antibodies) may be derived based on any of the BIOATLA® methods including, but not limited to, VERSITOPE™ Antibody Generation, natural diversity approaches, and high diversity approaches for generating monoclonal antibodies, methods for generation of conditionally active polypeptides, humanized antibodies, mirac proteins, multi-specific antibodies or cross-species active mutant polypeptides, Comprehensive Integrated Antibody Optimization (CIAO!™), Comprehensive Positional Evolution (CPE™), Synergy Evolution, Flex Evolution, Comprehensive Positional Insertion Evolution (CPI™), Comprehensive Positional Deletion Evolution (CPD™), Combinatorial Protein Synthesis (CPS™), or any combination thereof. These methods are described in U.S. Pat. Nos. 8,859,467 and 8,709,755 and United States Publication Nos. US20130281303, US20130303399, US20150065690, US20150252119, US20150086562 and US20100138945, and International Publication Nos. WO2015105888, WO2012009026, WO2011109726, WO2016036916, and WO2016033331, the contents of each of which are herein incorporated by reference in their entirety.

In some embodiments, antibodies of the present disclosure are generated by any of the aforementioned means to target one or more of the following epitopes of the tau protein; phosphorylated tau peptides, pS396, pS396-pS404, pS404, pS396-pS404-pS422, pS422, pS199, pS199-pS202, pS202, pT181, pT231, cis-pT231, any of the following acetylated sites acK174, acK274, acK280, acK281 and/or any combination thereof

### Antibody Fragments and Variants

In some embodiments, antibody fragments encoded by payloads comprise antigen binding regions from intact antibodies. Examples of antibody fragments may include, but are not limited to Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab)₂ fragment that has two antigen-binding sites and is still capable of cross-linking antigen. Compounds and/or compositions of the present disclosure may comprise one or more of these fragments. For the purposes herein, an “antibody” may comprise a heavy and light variable domain as well as an Fe region.

In some embodiments, the Fc region may be a modified Fc region, as described in US Patent Publication US20150065690, wherein the Fc region may have a single amino acid substitution as compared to the corresponding sequence for the wild-type Fc region, wherein the single amino acid substitution yields an Fc region with preferred properties to those of the wild-type Fc region. Non-limiting examples of Fc properties that may be altered by the single amino acid substitution include bind properties or response to pH conditions

As used herein, the term “native antibody” refers to a usually heterotetrameric glycoprotein of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11): 2151-62 and Li, A. et al., 2004. Blood. 103 (12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia Pa. 2012. Ch. 3, p 47-54, the contents of which are herein incorporated by reference in their entirety). Determining residues making up CDRs may include the use of numbering schemes including, but not limited to, those taught by Kabat [Wu, T. T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989) and Al-Lazikani, B. et al., 1997, J. Mol. Biol. 273(4):927-48, the contents of each of which are herein incorporated by reference in their entirety], Lefranc (Lefranc, M. P. et al., 2005, Immunome Res. 1:3) and Honegger (Honegger, A. and Pluckthun, A. 2001. J. Mol. Biol. 309(3):657-70, the contents of which are herein incorporated by reference in their entirety).

V_{H} and V_{L} domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2, and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, D. et al., 2014. Peer J. 2:e456; the contents of which are herein incorporated by reference in their entirety). In some cases. CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia Pa. 2012. Ch. 3, p 47-54, the contents of which are herein incorporated by reference in their entirety).

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia Pa. 2012. Ch. 3, p 46-47, the contents of which are herein incorporated by reference in their entirety).

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of V_{H} and V_{L} antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

As used herein, the term “bispecific antibody” refers to an antibody capable of binding two different antigens. Such antibodies typically comprise regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

As used herein, the term “diabody” refers to a small antibody fragment with two antigen-binding sites. Diabodies comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404097; WO 9311161; and Hollinger et al. (Hollinger, P. et al., “Diabodies”: Small bivalent and bispecific antibody fragments. PNAS. 1993. 90:6444-8) the contents of each of which are incorporated herein by reference in their entirety.

The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling, and cell division. In some embodiments, methods of the present disclosure may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity.

In some embodiments, viral genomes of the present disclosure may encode antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, Centyrins, DARPINS™, fynomers, Kunitz domains, and domain peptides. In other embodiments, antibody mimetics may include one or more non-peptide regions.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition, or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

### Multispecific Antibodies

In some embodiments, payloads may encode antibodies that bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

In some embodiments, multi-specific antibodies may be prepared by the methods used by BIOATLA® and described in International Patent publication WO201109726, the contents of which are herein incorporated by reference in their entirety. First a library of homologous, naturally occurring antibodies is generated by any method known in the art (i.e., mammalian cell surface display), then screened by FACSAria or another screening method, for multi-specific antibodies that specifically bind to two or more target antigens. In some embodiments, the identified multi-specific antibodies are further evolved by any method known in the art, to produce a set of modified multi-specific antibodies. These modified multi-specific antibodies are screened for binding to the target antigens. In some embodiments, the multi-specific antibody may be further optimized by screening the evolved modified multi-specific antibodies for optimized or desired characteristics.

In some embodiments, multi-specific antibodies may be prepared by the methods used by BIOATLA® and described in Unites States Publication No. US20150252119, the contents of which are herein incorporated by reference in their entirety. In one approach, the variable domains of two parent antibodies, wherein the parent antibodies are monoclonal antibodies are evolved using any method known in the art in a manner that allows a single light chain to functionally complement heavy chains of two different parent antibodies. Another approach requires evolving the heavy chain of a single parent antibody to recognize a second target antigen. A third approach involves evolving the light chain of a parent antibody so as to recognize a second target antigen. Methods for polypeptide evolution are described in International Publication WO2012009026, the contents of which are herein incorporated by reference in their entirety, and include as non-limiting examples, Comprehensive Positional Evolution (CPE), Combinatorial Protein Synthesis (CPS), Comprehensive Positional Insertion (CPI), Comprehensive Positional Deletion (CPD), or any combination thereof. The Fc region of the multi-specific antibodies described in United States Publication No. US20150252119 may be created using a knob-in-hole approach, or any other method that allows the Fc domain to form heterodimers. The resultant multi-specific antibodies may be further evolved for improved characteristics or properties such as binding affinity for the target antigen.

### Bispecific Antibodies

In some embodiments, payloads may encode bispecific antibodies. Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen.

In some cases, payloads encode bispecific antibodies comprising antigen-binding regions from two different anti-tau antibodies. For example, such bispecific antibodies may comprise binding regions from two different antibodies selected from Table 3.

Bispecific antibody frameworks may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fe receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs. 2010. January-February; 2(1):77-83).

In some embodiments, payloads may encode antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (V_{HH}s) of heavy chain antibodies found in camels and llamas, which lack light chains (Nelson, A. L., MAbs. 2010. January-February; 2(1):77-83).

Disclosed and claimed in PCT Publication WO2014144573 to Memorial Sloan-Kettering Cancer Center are multimerization technologies for making dimeric multispecific binding agents (e.g., fusion proteins comprising antibody components) with improved properties over multispecific binding agents without the capability of dimerization.

In some cases, payloads may encode tetravalent bispecific antibodies (TetBiAbs as disclosed and claimed in PCT Publication WO2014144357). TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

In some aspects, payloads may encode biosynthetic antibodies as described in U.S. Pat. No. 5,091,513, the contents of which are herein incorporated by reference in their entirety. Such antibody may include one or more sequences of amino acids constituting a region which behaves as a biosynthetic antibody binding site (BABS). The sites comprise 1) non-covalently associated or disulfide bonded synthetic V_{H} and V_{L} dimers, 2) V_{H}-V_{L} or V_{L}-V_{H} single chains wherein the V_{H} and V_{L} are attached by a polypeptide linker, or 3) individuals V_{H} or V_{L} domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, payloads may encode antibodies with antibody acceptor frameworks taught in U.S. Pat. No. 8,399,625. Such antibody acceptor frameworks may be particularly well suited accepting CDRs from an antibody of interest. In some cases, CDRs from anti-tau antibodies known in the art or developed according to the methods presented herein may be used.

### Miniaturized Antibody

In some embodiments, the antibody encoded by the payloads may be a “miniaturized” antibody. Among the best examples of mAb miniaturization are the small modular immunopharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE, and possibly multiple sclerosis, although these projects remain in the earliest stages of clinical testing. (Nelson, A. L., MAbs. 2010. January-February; 2(1):77-83).

### Diabodies

In some embodiments, payloads may encode diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al. Proc. Natl. Acad. Si., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs., 2010. January-February; 2(1):77-83).

### Unibody

In some embodiments, payloads may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Other antibodies may be “miniaturized” antibodies, which are compacted 100 kDa antibodies (see, e.g., Nelson. A. L., MAbs., 2010. January-February; 2(1):77-83).

### Intrabodies

In some embodiments, payloads may encode intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes using methods known in the art, such as those disclosed and reviewed in: (Marasco et al., 1993 Proc. Natl. Acad. Sci. USA, 90: 7889-7893; Chen et al., 1994, Hum. Gene her. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998. FEBS Lett. 437:81-6; Richardson et al., 1998. Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger el al., 2000, Proc. Natl. Acad. Sci. USA 97:805-810). See generally Marasco, W A, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

Sequences from donor antibodies may be used to develop intrabodies. Intrabodies are often recombinantly expressed as single domain fragments such as isolated V_{H} and VL domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal, such as the KDEL amino acid motif (SEQ ID NO: 4545). Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly synthesized intrabody within the cell is affected by reducing conditions of the intracellular environment.

Intrabodies may be promising therapeutic agents for the treatment of misfolding diseases, including Tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site (Cardinale, and Biocca, Curr. Mol. Med. 2008, 8:2-11).

### Maxibodies

In some embodiments, the payloads encode a maxibody (bivalent scFv fused to the amino terminus of the Fc (CH2-CH3 domains) of IgG.

### Chimeric Antigen Receptors

In some embodiments, the polypeptides encoded by the viral genomes (e.g., antibodies) may be used to generate chimeric antigen receptors (CARs) as described by BIOATLA® in International Publications WO2016033331 and WO2016036916, the contents of which are herein incorporated by reference in their entirety. As used herein, a “chimeric antigen receptor (CAR)” refers to an artificial chimeric protein comprising at least one antigen specific targeting region (ASTR), wherein the antigen specific targeting region comprises a full-length antibody or a fragment thereof that specifically binds to a target antigen. The ASTR may comprise any of the following: a full length heavy or light chain, an Fab fragment, a single chain Fv fragment, a divalent single chain antibody, or a diabody. As a non-limiting example, the ASTR of a CAR may be any of the antibodies listed in Table 3, antibody-based compositions or fragments thereof. Any molecule that is capable of binding a target antigen with high affinity can be used in the ASTR of a CAR. In some embodiments, the CAR may have more than one ASTR. These ASTRs may target two or more antigens or two or more epitopes of the same antigen. In some embodiments, the CAR is conditionally active. In some embodiments, the CAR is used to produce a genetically engineered cytotoxic cell carrying the CAR and capable of targeting the antigen bound by the ASTR.

Chimeric antigen receptors (CARs) are particularly useful in the treatment of cancers, though also therapeutically effective in treatment of a wide variety of other diseases and disorders. Non-limiting examples of disease categories that may be treated with CARs or CAR-based therapeutics include autoimmune disorders, B-cell mediated diseases, inflammatory diseases, neuronal disorders, cardiovascular disease and circulatory disorders, or infectious diseases. Not wishing to be bound by theory, CARs traditionally work by targeting antigens presented on the surface of or on the inside of cells to be destroyed e.g., cancer tumor cells, by the cytotoxic cell of the CAR.

### Senescent Cell Surface Protein Antibodies

In some embodiments, the AAV particles may comprise nucleic acids which have been engineered to express of antibodies that selectively bind to surface marker proteins of senescent cells. For example, the antibodies may selectively bind to proteins that are in misfolded conformation. The binding antibodies may reduce the number of senescent cells and be used to treat age-related conditions, such as, but not limited to, Alzheimer's disease, cardiovascular disease, emphysema, sarcopenia, and tumorigenesis as well as conditions more cosmetic in nature such as signs of skin aging including wrinkling, sagging, discoloration, age-related tissue dysfunction, tumor formation, and other age-related conditions.

In some embodiments, the expressed antibodies binding to epitopes of senescent cell surface proteins may be, but are not limited to, such as prion epitopes presented by SEQ ID NO: 1-14 of International Publication No. WO2014186878; CD44 epitopes presented by SEQ ID NO: 47-51 of International Publication No. WO2014186878; TNFR epitopes presented by SEQ ID NO: 52-56 of International Publication No. WO2014186878; NOTCH1 epitope presented by SEQ ID NO: 57-61 of International Publication No. WO2014186878; FasR epitopes presented by SEQ ID NO: 62-66 of International Publication No. WO2014186878; epidermal growth factor epitopes presented by SEQ ID NO: 67-81 of International Publication No. WO2014186878; CD38 epitopes presented by SEQ ID NO: 82-86 of International Publication No. WO2014186878, the contents of each of which are herein incorporated by reference in their entirety.

In some embodiments, the expressed antibodies may comprise peptides binding to senescent cell surface prion proteins, such as, but not limited to, those presented by SEQ ID NO: 15-36 of International Publication No. WO2014186878, the contents of which are herein incorporated by reference in their entirety.

In some embodiments, the expressed antibody may be AMF-3a-118 or AMF 3d-19 (SEQ ID NO: 89-92 and 103-106 of International publication WO2014186878, respectively, the contents of which are herein incorporated by reference in their entirety) targeting senescent cell surface protein FasR. In some embodiments, the expressed antibody may be Ab c-120 (SEQ ID NO: 37-40 of International publication WO2014186878, the contents of which are herein incorporated by reference in their entirety) targeting senescent cell surface protein PrP.

### Payload Antibodies

In some embodiments, the payload region of the AAV particle comprises one or more nucleic acid sequences encoding tau associated disease antibodies, variants or fragments thereof.

In some embodiments, the payload region of the AAV particle comprises one or more nucleic acid sequences encoding one or more of the payload antibody polypeptides listed in Table 3, or variants or fragments thereof. As used herein, “antibody polynucleotide” refers to a nucleic acid sequence encoding an antibody polypeptide.

In some embodiments, the payload region of the AAV particle comprises one or more nucleic acid sequences listed in Table 3, or variants or fragments thereof.

In some embodiments, the payload region of the AAV particle comprises a nucleic acid sequence encoding a payload antibody with at least 50% identity to one or more payload antibody polypeptides listed in Tables 3. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 3, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 3, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 3, or variants or fragments thereof.

In some embodiments, the payload antibody has 90% identity to one or more of the antibody polypeptides listed in Table 3, or variants or fragments thereof.

In some embodiments, the payload antibody has 91% identity to one or more of the antibody polypeptides listed in Table 3, or variants or fragments thereof.

In some embodiments, the payload antibody has 92% identity to one or more of the antibody polypeptides listed in Table 3, or variants or fragments thereof.

In some embodiments, the payload antibody has 93% identity to one or more of the antibody polypeptides listed in Table 3, or variants or fragments thereof.

In some embodiments, the payload antibody has 94% identity to one or more of the antibody polypeptides listed in Table 3, or variants or fragments thereof.

In some embodiments, the payload antibody has 95% identity to one or more of the antibody polypeptides listed in Table 3, or variants or fragments thereof.

In some embodiments, the payload antibody has 96% identity to one or more of the antibody polypeptides listed in Table 3, or variants or fragments thereof.

In some embodiments, the payload antibody has 97% identity to one or more of the antibody polypeptides listed in Table 3, or variants or fragments thereof.

In some embodiments, the payload antibody has 98% identity to one or more of the antibody polypeptides listed in Table 3, or variants or fragments thereof.

In some embodiments, the payload antibody has 99% identity to one or more of the antibody polypeptides listed in Table 3, or variants or fragments thereof.

In some embodiments, the payload antibody has 100% identity to one or more of the antibody polypeptides listed in Table 3, or variants or fragments thereof.

In some embodiments, the payload region of the AAV particle comprises a nucleic acid sequence with at least 50% identity to one or more nucleic acid sequences listed in Table 3, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 3, or variants or fragments thereof.

In some embodiments, the payload nucleic acid sequence has 90% identity to one or more of the nucleic acid sequences listed in Table 3, or variants or fragments thereof.

In some embodiments, the payload nucleic acid sequence has 91% identity to one or more of the nucleic acid sequences listed in Table 3, or variants or fragments thereof.

In some embodiments, the payload nucleic acid sequence has 92% identity to one or more of the nucleic acid sequences listed in Table 3, or variants or fragments thereof.

In some embodiments, the payload nucleic acid sequence has 93% identity to one or more of the nucleic acid sequences listed in Table 3, or variants or fragments thereof.

In some embodiments, the payload nucleic acid sequence has 94% identity to one or more of the nucleic acid sequences listed in Table 3, or variants or fragments thereof.

In some embodiments, the payload nucleic acid sequence has 95% identity to one or more of the nucleic acid sequences listed in Table 3, or variants or fragments thereof.

In some embodiments, the payload nucleic acid sequence has 96% identity to one or more of the nucleic acid sequences listed in Table 3, or variants or fragments thereof.

In some embodiments, the payload nucleic acid sequence has 97% identity to one or more of the nucleic acid sequences listed in Table 3, or variants or fragments thereof.

In some embodiments, the payload nucleic acid sequence has 98% identity to one or more of the nucleic acid sequences listed in Table 3, or variants or fragments thereof.

In some embodiments, the payload nucleic acid sequence has 99% identity to one or more of the nucleic acid sequences listed in Table 3, or variants or fragments thereof.

In some embodiments, the payload nucleic acid sequence has 100% identity to one or more of the nucleic acid sequences listed in Table 3, or variants or fragments thereof.

TABLE 3
Tau Associated Disease Antibodies

| | | Type | | | | SEQ |
|---|---|---|---|---|---|---|
| | | (PRT/ | | | | ID |
| Ab ID | Component | DNA) | Description | Reference | Len. | NO |
| Ab1 | VH Signal | DNA | IPN002 (VH Signal) | | 57 | 1740 |
| Ab2 | VH Signal | DNA | PHF1 (VH Signal) | | 57 | 1741 |
| Ab3 | VH Constant | DNA | VH (constant - 969) | | 969 | 1742 |
| Ab4 | VH Constant | DNA | VH (constant - 972) | | 972 | 1743 |
| Ab5 | VH CDR | PRT | 7295-M6 HCDR | WO2015122922; SEQ ID | 10 | 1744 |
| | | | | NO: 12 and 20 | | |
| Ab6 | VH CDR | PRT | 7295-M6 HCDR | WO2015122922; SEQ ID | 16 | 1745 |
| | | | | NO: 13 and 21 | | |
| Ab7 | VH CDR | PRT | 7295-M6 HCDR | WO2015122922; SEQ ID | 4 | 1746 |
| | | | | NO: 14 and 22 | | |
| Ab8 | VH CDR | PRT | 7297-2M1 HCDR | WO2015122922; SEQ ID | 16 | 1747 |
| | | | | NO: 69 | | |
| Ab9 | VH CDR | PRT | 7298-M1 HCDR | WO2015122922; SEQ ID | 11 | 1748 |
| | | | | NO: 28 | | |
| Ab10 | VH CDR | PRT | 7298-M1 HCDR | WO2015122922; SEQ ID | 16 | 1749 |
| | | | | NO: 29 | | |
| Ab11 | VH CDR | PRT | 7298-M1 HCDR | WO2015122922; SEQ ID | 4 | 1750 |
| | | | | NO: 30 | | |
| Ab12 | VH CDR | PRT | 7298-M2 HCDR | WO2015122922; SEQ ID | 10 | 1751 |
| | | | | NO: 36 | | |
| Ab13 | VH CDR | PRT | 7298-M2 HCDR | WO2015122922; SEQ ID | 14 | 1752 |
| | | | | NO: 37 | | |
| Ab14 | VH CDR | PRT | 7298-M2 HCDR | WO2015122922; SEQ ID | 8 | 1753 |
| | | | | NO: 38 | | |
| Ab15 | VH CDR | PRT | 7299-M2 HCDR | WO2015122922; SEQ ID | 10 | 1754 |
| | | | | NO: 44 and 60 | | |
| Ab16 | VH CDR | PRT | 7299-M2 HCDR | WO2015122922; SEQ ID | 16 | 1755 |
| | | | | NO: 45 | | |
| Ab17 | VH CDR | PRT | 7299-M2 HCDR | WO2015122922; SEQ ID | 4 | 1756 |
| | | | | NO: 46 and 62 | | |
| Ab18 | VH CDR | PRT | 7299-M5 HCDR | WO2015122922; SEQ ID | 10 | 1757 |
| | | | | NO: 52 and 68 | | |
| Ab19 | VH CDR | PRT | 7299-M5 HCDR | WO2015122922; SEQ ID | 16 | 1758 |
| | | | | NO: 53 | | |
| Ab20 | VH CDR | PRT | 7299-M5 HCDR | WO2015122922; SEQ ID | 10 | 1759 |
| | | | | NO: 54 and 70 | | |
| Ab21 | VH CDR | PRT | 7299-M9 HCDR | WO2015122922; SEQ ID | 15 | 1760 |
| | | | | NO: 61 | | |
| Ab22 | VH CDR | PRT | C10.2 HCDR1 | WO2017009308; SEQ ID | 13 | 1761 |
| | | | | NO: 12, 20 and 28 | | |
| Ab23 | VH CDR | PRT | C10.2 HCDR2 | WO2017009308; SEQ ID | 16 | 1762 |
| | | | | NO: 13 and 29 | | |
| Ab24 | VH CDR | PRT | C10.2 HCDR3 | WO2017009308; SEQ ID | 6 | 1763 |
| | | | | NO: 14 and 30 | | |
| Ab25 | VH CDR | PRT | C5.2 HCDR2 | WO2017009308; SEQ ID | 16 | 1764 |
| | | | | NO: 21 | | |
| Ab26 | VH CDR | PRT | C5.2 HCDR3 | WO2017009308; SEQ ID | 6 | 1765 |
| | | | | NO: 22 | | |
| Ab27 | VH CDR | PRT | D1.2 HCDR1 | WO2017009308; SEQ ID | 13 | 1766 |
| | | | | NO: 4 | | |
| Ab28 | VH CDR | PRT | D1.2 HCDR2 | WO2017009308; SEQ ID | 16 | 1767 |
| | | | | NO: 5 | | |
| Ab29 | VH CDR | PRT | D1.2 HCDR3 | WO2017009308; SEQ ID | 6 | 1768 |
| | | | | NO: 6 | | |
| Ab30 | VH CDR | PRT | HCDR1 | U.S. Pat. No. 8,980,270; | 5 | 1769 |
| | | | | SEQ ID NO: 4 | | |
| Ab31 | VH CDR | PRT | HCDR1 | U.S. Pat. No. 8,980,270; | 5 | 1770 |
| | | | | SEQ ID NO: 10 | | |
| Ab32 | VH CDR | PRT | HCDR2 | U.S. Pat. No. 8,980,270; | 17 | 1771 |
| | | | | SEQ ID NO: 5 | | |
| Ab33 | VH CDR | PRT | HCDR2 | U.S. Pat. No. 8,980,270; | 17 | 1772 |
| | | | | SEQ ID NO: 11 | | |
| Ab34 | VH CDR | PRT | HCDR2 | WO2015081085; SEQ ID | 32 | 1773 |
| | | | | NO: 27 | | |
| Ab35 | VH CDR | PRT | HCDR3 | U.S. Pat. No. 8,980,270; | 8 | 1774 |
| | | | | SEQ ID NO: 6 | | |
| Ab36 | VH CDR | PRT | HCDR3 | U.S. Pat. No. 8,980,270; | 8 | 1775 |
| | | | | SEQ ID NO: 12 | | |
| Ab37 | VH CDR | PRT | HCDR3 | WO2015081085; SEQ ID | 11 | 1776 |
| | | | | NO: 44 | | |
| Ab38 | VH CDR | PRT | IPN002 HCDR1 | WO2014028777; SEQ ID | 14 | 1777 |
| | | | | NO: 84 | | |
| Ab39 | VH CDR | PRT | IPN002 HCDR2 | WO2014028777; SEQ ID | 32 | 1778 |
| | | | | NO: 85 | | |
| Ab40 | VH CDR | PRT | IPN002 HCDR3 | WO2014028777; SEQ ID | 11 | 1779 |
| | | | | NO: 86 | | |
| Ab41 | VH CDR | PRT | MC1 LCDR1 | WO2016137811; SEQ ID | 13 | 1780 |
| | | | | NO: 6 | | |
| Ab42 | VH CDR | PRT | MC1 LCDR2 | WO2016137811; SEQ ID | 18 | 1781 |
| | | | | NO: 7 | | |
| Ab43 | VH CDR | PRT | MC1 LCDR3 | WO2016137811; SEQ ID | 9 | 1782 |
| | | | | NO: 8 | | |
| Ab44 | VH CDR | PRT | PT1 HCDR1 (Chothia) | U.S. Pat. No. 9,371,376; | 7 | 1783 |
| | | | | SEQ ID NO: 19 | | |
| Ab45 | VH CDR | PRT | PT1 HCDR1 (Rabat) | U.S. Pat. No. 9,371,376; | 5 | 1784 |
| | | | | SEQ ID NO: 7 | | |
| Ab46 | VH CDR | PRT | PT1 HCDR2 (Chothia) | U.S. Pat. No. 9,371,376; | 6 | 1785 |
| | | | | SEQ ID NO: 20 | | |
| Ab47 | VH CDR | PRT | PT1 HCDR2 (Rabat) | U.S. Pat. No. 9,371,376; | 17 | 1786 |
| | | | | SEQ ID NO. 8 | | |
| Ab48 | VH CDR | PRT | PT1 HCDR3 (Chothia) | U.S. Pat. No. 9,371,376; | 9 | 1787 |
| | | | | SEQ ID NO: 21 | | |
| Ab49 | VH CDR | PRT | PT1 HCDR3 (Rabat) | U.S. Pat. No. 9,371,376; | 10 | 1788 |
| | | | | SEQ ID NO: 9 | | |
| Ab50 | VH CDR | PRT | PT3 HCDR I (Chothia) | U.S. Pat. No. 9,371,376; | 7 | 1789 |
| | | | | SEQ ID NO: 25 | | |
| Ab51 | VH CDR | PRT | PT3 HCDR1 (Rabat) | U.S. Pat. No. 9,371,376; | 5 | 1790 |
| | | | | SEQ ID NO: 13 | | |
| Ab52 | VH CDR | PRT | PT3 HCDR2 (Chothia) | U.S. Pat. No. 9,371,376; | 5 | 1791 |
| | | | | SEQ ID NO: 26 | | |
| Ab53 | VH CDR | PRT | PT3 HCDR2 (Rabat) | U.S. Pat. No. 9,371,376; | 16 | 1792 |
| | | | | SEQ ID NO: 14 | | |
| Ab54 | VH CDR | PRT | PT3 HCDR3 (Chothia) | U.S. Pat. No. 9,371,376; | 9 | 1793 |
| | | | | SEQ ID NO: 27 | | |
| Ab55 | VH CDR | PRT | PT3 HCDR3 (Rabat) | U.S. Pat. No. 9,371,376; | 10 | 1794 |
| | | | | SEQ ID NO: 15 | | |
| Ab56 | VH | PRT | 7295-M6 VH | WO2015122922; SEQ ID | 125 | 1795 |
| | | | | NO: 16 and 24 | | |
| Ab57 | VH | PRT | 7297-2M1 VH | WO2015122922; SEQ ID | 131 | 1796 |
| | | | | NO: 72 | | |
| Ab58 | VH | PRT | 7298-M1 VH | WO2015122922; SEQ ID | 127 | 1797 |
| | | | | NO: 32 | | |
| Ab59 | VH | PRT | 7298-M2 VH | WO2015122922; SEQ ID | 128 | 1798 |
| | | | | NO: 40 | | |
| Ab60 | VH | PRT | 7299-M2 VH | WO2015122922; SEQ ID | 125 | 1799 |
| | | | | NO: 48 | | |
| Ab61 | VH | PRT | 7299-M5 VH | WO2015122922; SEQ ID | 131 | 1800 |
| | | | | NO: 56 | | |
| Ab62 | VH | PRT | 7299-M9 VH | WO2015122922; SEQ ID | 125 | 1801 |
| | | | | NO: 64 | | |
| Ab63 | VH | PRT | C10.2 VH | WO2017009308; SEQ ID | 439 | 1802 |
| | | | | NO: 16 | | |
| Ab64 | VH | PRT | C10.2 VH - 115 | | 115 | 1803 |
| Ab65 | VH | DNA | C10.2 VH | | 345 | 1804 |
| | | | Bioinformatics | | | |
| Ab66 | VH | DNA | C10.2 VH EMBOSS | | 345 | 1805 |
| Ab67 | VH | DNA | C10.2 VH Geneinfinity | | 345 | 1806 |
| Ab68 | VH | DNA | C10.2 VH | | 345 | 1807 |
| | | | GregThatcher | | | |
| Ab69 | VH | DNA | C10.2 VH IDT | | 345 | 1808 |
| Ab70 | VH | DNA | C10.2 VH InSilico | | 345 | 1809 |
| Ab71 | VH | DNA | C10.2 VH MolBio | | 345 | 1810 |
| Ab72 | VH | DNA | C10.2 VH N2P | | 345 | 1811 |
| Ab73 | VH | DNA | C10.2 VH Snapgene | | 345 | 1812 |
| Ab74 | VH | DNA | C10.2 VH Vector NTT | | 345 | 1813 |
| Ab75 | VH | PRT | C5.2 VH | WO2017009308; SEQ ID | 439 | 1814 |
| | | | | NO: 24 | | |
| Ab76 | VH | PRT | C8.3 VH | WO2017009308; SEQ ID | 439 | 1815 |
| | | | | NO: 32 | | |
| Ab77 | VH | PRT | D1.2 VH | WO2017009308; SEQ ID | 451 | 1816 |
| | | | | NO: 8 | | |
| Ab78 | VH | PRT | hC10.2 VH | WO2017009308; SEQ ID | 444 | 1817 |
| | | | | NO: 35 | | |
| Ab79 | VH | DNA | IPN001 VH - 351 | U.S. Pat. No. 8,980,270; | 351 | 1818 |
| | | | | SEQ ID NO: 18 | | |
| Ab80 | VH | PRT | IPN001 VH - 117 | U.S. Pat. No. 8,980,270; | 117 | 1819 |
| | | | | SEQ ID NO: 14 | | |
| Ab81 | VH | DNA | IPN002 VH - 1377 | | 1377 | 1820 |
| Ab82 | VH | DNA | IPN002 VH - 351 | U.S. Pat. No. 8,980,270; | 351 | 1821 |
| | | | | SEQ ID NO: 20 | | |
| Ab83 | VH | PRT | IPN002 VH - 117 | U.S. Pat. No. 8,980,270; | 117 | 1822 |
| | | | | SEQ ID NO: 16 | | |
| Ab84 | VH | PRT | IPN002 VH - 30 | WO2014028777; SEQ ID | 30 | 1823 |
| | | | | NO: 83 | | |
| Ab85 | VH | DNA | IPN002 VH variant 1 | U.S. Pat. No. 8,980,270; | 351 | 1824 |
| | | | | SEQ ID NO: 28 | | |
| Ab86 | VH | PRT | IPN002 VH variant 1 | U.S. Pat. No. 8,980,270; | 117 | 1825 |
| | | | | SEQ ID NO: 36 | | |
| Ab87 | VH | DNA | IPN002 VH variant 2 | U.S. Pat. No. 8,980,270; | 351 | 1826 |
| | | | | SEQ ID NO: 29 | | |
| Ab88 | VH | PRT | IPN002 VH variant 2 | U.S. Pat. No. 8,980,270; | 117 | 1827 |
| | | | | SEQ ID NO: 37 | | |
| Ab89 | VH | DNA | IPN002 VH variant 3 | U.S. Pat. No. 8,980,270; | 351 | 1828 |
| | | | | SEQ ID NO: 30 | | |
| Ab90 | VH | PRT | IPN002 VH variant 3 | U.S. Pat. No. 8,980,270; | 117 | 1829 |
| | | | | SEQ ID NO: 38 | | |
| Ab91 | VH | DNA | IPN002 VH variant 4 | U.S. Pat. No. 8,980,270; | 351 | 1830 |
| | | | | SEQ ID NO: 31 | | |
| Ab92 | VH | PRT | IPN002 VH variant 4 | U.S. Pat. No. 8,980,270; | 117 | 1831 |
| | | | | SEQ ID NO: 39 | | |
| Ab93 | VH | DNA | MC1 VH - 1374 | | 1374 | 1832 |
| Ab94 | VH | DNA | MC1 VH - 1326 | WO2016137811; SEQ ID | 1326 | 1833 |
| | | | | NO: 12 | | |
| Ab95 | VH | PRT | MC1 VH - 458 | | 458 | 1834 |
| Ab96 | VH | PRT | MC1 VH - 442 | WO2016137811; SEQ ID | 442 | 1835 |
| | | | | NO: 2 | | |
| Ab97 | VH | PRT | MC1 VH - 116 | WO2016137811; SEQ ID | 116 | 1836 |
| | | | | NO: 10 | | |
| Ab98 | VH | DNA | PHF1 VH - 1320 | | 1320 | 1837 |
| Ab99 | VH | DNA | PHF1 VH - 1377 | | 1377 | 1838 |
| Ab100 | VH | DNA | PHF1 VH - 351 | | 351 | 1839 |
| Ab101 | VH | DNA | PHF1 VH - 699 | | 699 | 1840 |
| Ab102 | VH | DNA | PHF1 VH - 717 | | 717 | 1841 |
| Ab103 | VH | DNA | PHF1 VH - 744 | | 744 | 1842 |
| Ab104 | VH | DNA | PHF1 VH - 753 | | 753 | 1843 |
| Ab105 | VH | PRT | PHF1 VH - 459 | | 459 | 1844 |
| Ab106 | VH | DNA | PHF1 VH+ | | 1383 | 1845 |
| Ab107 | VH | DNA | PT1 VH - 357 | U.S. Pat. No. 9,371,376; | 357 | 1846 |
| | | | | SEQ ID NO: 31 | | |
| Ab108 | VH | PRT | PT1 VH - 119 | U.S. Pat. No. 9,371,376; | 119 | 1847 |
| | | | | SEQ ID NO: 35 | | |
| Ab109 | VH | DNA | PT3 VH - 354 | U.S. Pat. No. 9,371,376; | 354 | 1848 |
| | | | | SEQ ID NO: 33 | | |
| Ab110 | VH | PRT | PT3 VH - 118 | U.S. Pat. No. 9,371,376; | 118 | 1849 |
| | | | | SEQ ID NO: 37 | | |
| Ab111 | VH | DNA | PT3 VH Bioinformatics | | 354 | 1850 |
| Ab112 | VH | DNA | PT3 VH Bioinformatics | | 354 | 1851 |
| | | | 2 | | | |
| Ab113 | VH | DNA | PT3 VH EMBOSS | | 354 | 1852 |
| Ab114 | VH | DNA | PT3 VH Geneinfinity | | 354 | 1853 |
| Ab115 | VH | DNA | PT3 VH Genescript | | 354 | 1854 |
| Ab116 | VH | DNA | PT3 VH IDT | | 354 | 1855 |
| Ab117 | VH | DNA | PT3 VH NUS | | 354 | 1856 |
| Ab118 | VH | DNA | PT3 VH NUS2 | | 354 | 1857 |
| Ab119 | VH | DNA | PT3 VH NUS3 | | 354 | 1858 |
| Ab120 | VH | DNA | PT3 VH Snapgene | | 354 | 1859 |
| Ab121 | VH | PRT | VH | WO2015081085; SEQ ID | 30 | 1860 |
| | | | | NO: 25 | | |
| Ab122 | VL Signal | DNA | IPN002 (VL Signal) | | 57 | 1861 |
| Ab123 | VL Signal | DNA | PHF1 (VL Signal) | | 57 | 1862 |
| Ab124 | VL Signal | DNA | PHF1 (VL Signal 72) | | 72 | 1863 |
| Ab125 | VL Constant | DNA | VL (constant) | | 321 | 1864 |
| Ab126 | VL CDR | PRT | 7295-M6 LCDR | WO2015122922; SEQ ID | 16 | 1865 |
| | | | | NO: 9 and 17 | | |
| Ab127 | VL CDR | PRT | 7295-M6 LCDR | WO2015122922; SEQ ID | 9 | 1866 |
| | | | | NO: 11 and 19 | | |
| Ab128 | VL CDR | PRT | 7297-2M1 LCDR | WO2015122922; SEQ ID | 11 | 1867 |
| | | | | NO: 65 | | |
| Ab129 | VL CDR | PRT | 7297-2M1 LCDR | WO2015122922; SEQ ID | / | 1868 |
| | | | | NO: 66 | | |
| Ab130 | VL CDR | PRT | 7297-2M1 LCDR | WO2015122922; SEQ ID | 8 | 1869 |
| | | | | NO: 67 | | |
| Ab131 | VL CDR | PRT | 7298-M1 LCDR | WO2015122922; SEQ ID | 15 | 1870 |
| | | | | NO: 25 and 33 | | |
| Ab132 | VL CDR | PRT | 7298-M1 LCDR | WO2015122922; SEQ ID | 7 | 1871 |
| | | | | NO: 26 and 34 | | |
| Ab133 | VL CDR | PRT | 7298-M1 LCDR | WO2015122922; SEQ ID | 9 | 1872 |
| | | | | NO: 27 and 35 | | |
| Ab134 | VL CDR | PRT | 7299-M2 LCDR | WO2015122922; SEQ ID | 16 | 1873 |
| | | | | NO: 41 | | |
| Ab135 | VL CDR | PRT | 7299-M2 LCDR | WO2015122922; SEQ ID | 7 | 1874 |
| | | | | NO: 42 | | |
| Ab136 | VL CDR | PRT | 7299-M2 LCDR | WO2015122922; SEQ ID | 9 | 1875 |
| | | | | NO: 43 | | |
| Ab137 | VL CDR | PRT | 7299-M5 LCDR | WO2015122922; SEQ ID | 16 | 1876 |
| | | | | NO: 49 and 57 | | |
| Ab138 | VL CDR | PRT | 7299-M5 LCDR | WO2015122922; SEQ ID | 7 | 1877 |
| | | | | NO: 50 and 58 | | |
| Ab139 | VL CDR | PRT | 7299-M5 LCDR | WO2015122922; SEQ ID | 9 | 1878 |
| | | | | NO: 51 | | |
| Ab140 | VL CDR | PRT | 7299-M9 LCDR | WO2015122922; SEQ ID | 8 | 1879 |
| | | | | NO: 59 | | |
| Ab141 | VL CDR | PRT | C10.2 LCDR1 | WO2017009308; SEQ ID | 11 | 1880 |
| | | | | NO: 9 and 25 | | |
| Ab142 | VL CDR | PRT | C10.2 LCDR2 | WO2017009308; SEQ ID | 7 | 1881 |
| | | | | NO: 10 and 18 | | |
| Ab143 | VL CDR | PRT | C10.2 LCDR3 | WO2017009308; SEQ ID | 7 | 1882 |
| | | | | NO: 11 and 19 | | |
| Ab144 | VL CDR | PRT | C5.2 LCDR1 | WO2017009308; SEQ ID | 11 | 1883 |
| | | | | NO: 17 | | |
| Ab145 | VL CDR | PRT | C8.3 LCDR2 | WO2017009308; SEQ ID | / | 1884 |
| | | | | NO: 26 | | |
| Ab146 | VL CDR | PRT | C8.3 LCDR3 | WO2017009308; SEQ ID | 7 | 1885 |
| | | | | NO: 27 | | |
| Ab147 | VL CDR | PRT | D1.2 LCDR1 | WO2017009308; SEQ ID | 16 | 1886 |
| | | | | NO: 1 | | |
| Ab148 | VL CDR | PRT | D1.2 LCDR2 | WO2017009308; SEQ ID | 7 | 1887 |
| | | | | NO: 2 | | |
| Ab149 | VL CDR | PRT | D1.2 LCDR3 | WO2017009308; SEQ ID | 7 | 1888 |
| | | | | NO: 3 | | |
| Ab150 | VL CDR | PRT | IPN002 LCDR1 | WO2014028777; SEQ ID | 15 | 1889 |
| | | | | NO: 55 | | |
| Ab151 | VL CDR | PRT | IPN002 LCDR2 | WO2014028777; SEQ ID | 32 | 1890 |
| | | | | NO: 56 | | |
| Ab152 | VL CDR | PRT | IPN002 LCDR3 | WO2014028777; SEQ ID | 10 | 1891 |
| | | | | NO: 57 | | |
| Ab153 | VL CDR | PRT | LCDR1 | U.S. Pat. No. 8,980,270; | 16 | 1892 |
| | | | | SEQ ID NO: 1 | | |
| Ab154 | VL CDR | PRT | LCDR1 | U.S. Pat. No. 8,980,270; | 16 | 1893 |
| | | | | SEQ ID NO: 7 | | |
| Ab155 | VL CDR | PRT | LCDR1 | WO2015081085; SEQ ID | 15 | 1894 |
| | | | | NO: 46 | | |
| Ab156 | VL CDR | PRT | LCDR2 | U.S. Pat. No. 8,980,270; | 7 | 1895 |
| | | | | SEQ ID NO: 2 | | |
| Ab157 | VL CDR | PRT | LCDR2 | WO2015081085; SEQ ID | 32 | 1896 |
| | | | | NO: 47 | | |
| Ab158 | VL CDR | PRT | LCDR3 | U.S. Pat. No. 8,980,270; | 9 | 1897 |
| | | | | SEQ ID NO: 3 | | |
| | | | | and 9 | | |
| Ab159 | VL CDR | PRT | MC1 LCDR1 | WO2016137811; SEQ ID | 16 | 1898 |
| | | | | NO: 3 | | |
| Ab160 | VL CDR | PRT | MC1 LCDR2 | WO2016137811; SEQ ID | 8 | 1899 |
| | | | | NO: 4 | | |
| Ab161 | VL CDR | PRT | MC1 LCDR3 | WO2016137811; SEQ ID | 9 | 1900 |
| | | | | NO: 5 | | |
| Ab162 | VL CDR | PRT | PT1 LCDR1 (Chothia) | U.S. Pat No. 9,371,376; | 12 | 1901 |
| | | | | SEQ ID NO: 22 | | |
| Ab163 | VL CDR | PRT | PT1 LCDR1 (Rabat) | U.S. Pat No. 9,371,376; | 16 | 1902 |
| | | | | SEQ ID NO: 10 | | |
| Ab164 | VL CDR | PRT | PT1 LCDR2 (Chothia) | U.S. Pat No. 9,371,376; | 3 | 1903 |
| | | | | SEQ ID NO: 23 | | |
| Ab165 | VL CDR | PRT | PT1 LCDR2 (Rabat) | U.S. Pat No. 9,371,376; | 7 | 1904 |
| | | | | SEQ ID NO: 11 | | |
| Ab166 | VL CDR | PRT | PT1 LCDR3 (Chothia) | U.S. Pat No. 9,371,376; | 7 | 1905 |
| | | | | SEQ ID NO: 24 | | |
| Ab167 | VL CDR | PRT | PT1 LCDR3 (Rabat) | U.S. Pat No. 9,371,376; | 9 | 1906 |
| | | | | SEQ ID NO. 12 | | |
| Ab168 | VL CDR | PRT | PT3 LCDR1 (Chothia) | U.S. Pat No. 9,371,376; | 7 | 1907 |
| | | | | SEQ ID NO: 28 | | |
| Ab169 | VL CDR | PRT | PT3 LCDR1 (Rabat) | U.S. Pat No. 9,371,376; | 11 | 1908 |
| | | | | SEQ ID NO: 16 | | |
| Ab170 | VL CDR | PRT | PT3 LCDR2 (Chothia) | U.S. Pat No. 9,371,376; | 3 | 1909 |
| | | | | SEQ ID NO: 29 | | |
| Ab171 | VL CDR | PRT | PT3 LCDR2 (Rabat) | U.S. Pat No. 9,371,376; | 7 | 1910 |
| | | | | SEQ ID NO: 17 | | |
| Ab172 | VL CDR | PRT | PT3 LCDR3 (Chothia) | U.S. Pat No. 9,371,376; | 6 | 1911 |
| | | | | SEQ ID NO: 30 | | |
| Ab173 | VL CDR | PRT | PT3 LCDR3 (Rabat) | U.S. Pat No. 9,371,376; | 10 | 1912 |
| | | | | SEQ ID NO: 18 | | |
| Ab174 | VL | PRT | 7295-M6 VL | WO2015122922; SEQ ID | 122 | 1913 |
| | | | | NO: 15 and 23 | | |
| Ab175 | VL | PRT | 7297-2M1 VL | WO2015122922; SEQ ID | 116 | 1914 |
| | | | | NO: 71 | | |
| Ab176 | VL | PRT | 7298-M1 VL | WO2015122922; SEQ ID | 121 | 1915 |
| | | | | NO: 31 and 39 | | |
| Ab177 | VL | PRT | 7299-M2 VL | WO2015122922; SEQ ID | 122 | 1916 |
| | | | | NO: 47 | | |
| Ab178 | VL | PRT | 7299-M5 VL | WO2015122922; SEQ ID | 122 | 1917 |
| | | | | NO: 55 | | |
| Ab179 | VL | PRT | 7299-M9 VL | WO2015122922; SEQ ID | 122 | 1918 |
| | | | | NO: 63 | | |
| Ab180 | VL | PRT | C10.2 VL - 107 | | 107 | 1919 |
| Ab181 | VL | PRT | C10.2 VL -214 | WO2017009308; SEQ ID | 214 | 1920 |
| | | | | NO: 15 | | |
| Ab182 | VL | DMA | C10.2 VL | | 321 | 1921 |
| | | | Bioinformatics | | | |
| Ab183 | VL | DNA | C10.2 VL EMBOSS | | 321 | 1922 |
| Ab184 | VL | DNA | C10.2 VL GeneInfinity | | 321 | 1923 |
| Ab185 | VL | DNA | C10.2 VL | | 321 | 1924 |
| | | | GregThatcher | | | |
| Ab186 | VL | DNA | C10.2 VL IDT | | 321 | 1925 |
| Ab181 | VL | DNA | C10.2 VL InSilico | | 321 | 1926 |
| Ab188 | VL | DNA | C10.2 VL MolBio | | 321 | 1927 |
| Ab189 | VL | DNA | C10.2 VLN2P | | 321 | 1928 |
| Ab190 | VL | DNA | C10.2 VL Snapgene | | 321 | 1929 |
| Ab191 | VL | DNA | C10.2 VL Vector NTI | | 321 | 1930 |
| Ab192 | VL | PRT | C5.2. VL | WO2017009308; SEQ ID | 214 | 1931 |
| | | | | NO: 23 | | |
| Ab193 | VL | PRT | C8.3 VL | WO2017009308; SEQ ID | 214 | 1932 |
| | | | | NO: 31 | | |
| Ab194 | VL | PRT | D1.2 VL | WO2017009.308; SEQ ID | 219 | 1933 |
| | | | | NO: 7 | | |
| Ab195 | VL | PRT | D1.2* VL | WO2017009308; SEQ ID | 219 | 1934 |
| | | | | NO: 34 | | |
| Ab196 | VL | PRT | hC10.2 VL | WO2017009308; SEQ ID | 214 | 1935 |
| | | | | NO: 36 | | |
| Ab197 | VL | DNA | IPN001 VL - 336 | U.S. Pat No. 8,980,270; | 336 | 1936 |
| | | | | SEQ ID NO: 17 | | |
| Ab198 | VL | PRT | IPN001 VL | U.S. Pat No. 8,980,270; | 112 | 1937 |
| | | | | SEQ ID NO. 13 | | |
| Ab199 | VL | PRT | IPN002 VL | WO2014028777; SEQ ID | 23 | 1938 |
| | | | | NO: 54 | | |
| Ab200 | VL | DNA | IPN002 VL - 336 | U.S. Pat No. 8,980,270; | 336 | 1939 |
| | | | | SEQ ID NO. 19 | | |
| Ab201 | VL | DNA | IPN002 VL - 714 | | 714 | 1940 |
| Ab202 | VL | PRT | IPN002 VL - 112 | U.S. Pat No. 8,980,270; | 112 | 1941 |
| | | | | SEQ ID NO: 15 | | |
| Ab203 | VL | DNA | IPN002 VL variant 1 | U.S. Pat No. 8,980,270; | 336 | 1942 |
| | | | | SEQ ID NO: 32 | | |
| Ab204 | VL | PRT | IPN002 VL variant 1 | U.S. Pat No. 8,980,270; | 112 | 1943 |
| | | | | SEQ ID NO: 40 | | |
| Ab205 | VL | DNA | IPN002 VL variant 2 | U.S. Pat No. 8,980,270; | 336 | 1944 |
| | | | | SEQ ID NO: 33 | | |
| Ab206 | VL | PRT | IPN002 VL variant 2 | U.S. Pat No. 8,980,270; | 112 | 1945 |
| | | | | SEQ ID NO: 41 | | |
| Ab207 | VL | DNA | IPN002 VL variant 3 | U.S. Pat No. 8,980,270; | 336 | 1946 |
| | | | | SEQ ID NO: 34 | | |
| Ab208 | VL | PRT | IPN002 VL variant 3 | U.S. Pat No. 8,980,270; | 112 | 1947 |
| | | | | SEQ ID NO: 42 | | |
| Ab209 | VL | DNA | IPN002 VL variant 4 | U.S. Pat No. 8,980,270; | 336 | 1948 |
| | | | | SEQ ID NO: 35 | | |
| Ab210 | VL | PRT | IPN002 VL variant 4 | U.S. Pat No. 8,980,270; | 112 | 1949 |
| | | | | SEQ ID NO: 43 | | |
| Ab211 | VL | DNA | MC1 VL - 714 | | 714 | 1950 |
| Ab212 | VL | DNA | MC1 VL - 717 | | 717 | 1951 |
| Ab213 | VL | DNA | MC1 VL - 657 | WO2016137811; SEQ ID | 657 | 1952 |
| | | | | NO: 11 | | |
| Ab214 | VL | PRT | MC1 VL - 238 | | 238 | 1953 |
| Ab215 | VL | PRT | MC1 VL - 219 | WO2016137811; SEQ ID | 219 | 1954 |
| | | | | NO: 1 | | |
| Ab216 | VL | PRT | MC1 VL - 112 | WO2016137811; SEQ ID | 112 | 1955 |
| | | | | NO: 9 | | |
| Ab217 | VL | DNA | PHF1 VL - 339v1 | | 339 | 1956 |
| Ab218 | VL | DNA | PHF1 VL - 339v2 | | 339 | 1957 |
| Ab219 | VL | DNA | PHF1 VL - 396 | | 396 | 1958 |
| Ab220 | VL | DNA | PHF1 VL - 657 | | 657 | 1959 |
| Ab221 | VL | DNA | PHF1 VL - 660 | | 660 | 1960 |
| Ab222 | VL | DNA | PHF1 VL - 678 | | 678 | 1961 |
| Ab223 | VL | DNA | PHF1 VL - 714 | | 714 | 1962 |
| Ab224 | VL | DNA | PHF1 VL - 720 | | 720 | 1963 |
| Ab225 | VL | PRT | PHF1 VL - 239 | | 239 | 1964 |
| Ab226 | VL | DNA | PHF1 VL+ (732) | | 732 | 1965 |
| Ab227 | VL | DNA | PHF1 VL+ (747) | | 747 | 1966 |
| Ab228 | VL | DNA | PT1 VL - 336 | U.S. Pat No. 9,371,376; | 336 | 1967 |
| | | | | SEQ ID NO: 32 | | |
| Ab229 | VL | PRT | PT1 VL - 112 | U.S. Pat No. 9,371,376; | 112 | 1968 |
| | | | | SEQ ID NO: 36 | | |
| Ab230 | VL | DNA | PT3 VL - 321 | U.S. Pat No. 9,371,376; | 321 | 1969 |
| | | | | SEQ ID NO: 34 | | |
| Ab231 | VL | PRT | PT3 VL - 107 | U.S. Pat No. 9,371,376; | 107 | 1970 |
| | | | | SEQ ID NO: 38 | | |
| Ab232 | VL | DNA | PT3 VL Bioinformatics | | 321 | 1971 |
| Ab233 | VL | DNA | PT3 VL Bioinformatics | | 321 | 1972 |
| | | | 2 | | | |
| Ab234 | VL | DNA | PT3 VL EMBOSS | | 321 | 1973 |
| Ab235 | VL | DNA | PT3 VL Geneinfinity | | 321 | 1974 |
| Ab236 | VL | DNA | PT3 AT Genescript | | 321 | 1975 |
| Ab237 | VL | DNA | PT3 VL IDT | | 321 | 1976 |
| Ab238 | VL | DNA | PT3 VL NUS | | 321 | 1977 |
| Ab239 | VL | DNA | PT3 VL NUS2 | | 321 | 1978 |
| Ab240 | VL | DNA | PT3 VL NUS3 | | 321 | 1979 |
| Ab241 | VL | DNA | PT3 VL Snapgene | | 321 | 1980 |
| Ab242 | VL | PRT | VL | WO2015081085; SEQ ID | 23 | 1981 |
| | | | | NO: 45 | | |
| Ab243 | Fab | PRT | AT8 Fab1 | Malia, T. J. et al, “Epitope | 18 | 1982 |
| | | | | mapping and structural basis | | |
| | | | | for the recognition of | | |
| | | | | phosphorylated tan by the | | |
| | | | | anti-tau antibody AT8”, | | |
| | | | | Proteins 84 (4), 427-434 | | |
| | | | | (2016), Accession number | | |
| | | | | 5E2V_P | | |
| Ab244 | Fab | PRT | AT8 Fab2 | Malta, T. J. et al, “Epitope | 18 | 1983 |
| | | | | mapping and structural basis | | |
| | | | | for the recognition of | | |
| | | | | phosphorylated tau by the | | |
| | | | | anti-tau antibody AT8”, | | |
| | | | | Proteins 84 (4), 427-434 | | |
| | | | | (2016), Accession number | | |
| | | | | 5E2W_P | | |
| Ab245 | Full | DNA | MC1 Furin 2A - 2168 | WO2015035190; SEQ ID | 2168 | 1984 |
| | antibody | | | NO: 2 | | |
| Ab246 | Full | PRT | MC1 Furin 2A - 718 | WO2015035190; SEQ ID | 718 | 1985 |
| | antibody | | | NO: 4 | | |
| Ab247 | Full | DNA | MC1 optimized seq | WQ2015035190; SEQ ID | 2168 | 1986 |
| | antibody | | | NO: 6 | | |
| Ab248 | Full | DNA | PHF1 Furin 2A - 2174 | W02015035190; SEQ ID | 2174 | 1987 |
| | antibody | | | NO: 1 | | |
| Ab249 | Full | PRT | PHF1 Furin 2A - 726 | W02015035190; SEQ ID | 720 | 1988 |
| | antibody | | | NO: 3 | | |
| Ab250 | Full | DNA | PHF1 optimized seq | W02015035190; SEQ ID | 2174 | 1989 |
| | antibody | | | NO: 5 | | |
| Ab251 | VH | DNA | PT3 | | 348 | 2241 |
| Ab252 | VH Constant | DNA | VH (constant - 309) | | 309 | 2242 |
| Ab253 | VH Constant | DNA | VH (constant - 291) | | 291 | 2243 |
| Ab254 | VH | DNA | PHF1 VH-435 | | 435 | 2169 |
| Ab255 | VH | DNA | PHF1 VH-408 | | 408 | 2170 |

In some embodiments, tube payload region of the AAV particle comprises a nucleic acid sequence encoding a polypeptide which is an antibody, an antibody-based composition, or a fragment thereof. As a non-limiting example, the antibody may be one or more of the polypeptides listed in Table 3, or variants or fragments thereof. As another non-limiting example, the antibody may be one or more of the heavy chain sequences listed in Table 3. As a non-limiting example, the antibody may be one or more of the light chain sequences listed in Table 3, or variants or fragments thereof.

In some embodiments, the payload region of the AAV particle comprises a nucleic acid sequence encoding a polypeptide comprising a heavy chain and a light chain sequence listed in Table 3, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and a light chain sequence listed in Table 3, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 3, a linker from Table 2 and a heavy chain sequence from Table 3. Non-limiting examples are included in Tables 4-6 and Tables 15-91.

In some embodiments, the payload region comprises, in the 5′ to 3′ direction, an antibody heavy chain sequence, a linker region (may comprise one or more linkers) and a light chain sequence. In another embodiment, the linker is not used.

In some embodiments, the payload region comprises a nucleic acid sequence encoding, in the 5′ to 3′ direction, an antibody heavy chain sequence from Table 3, one or more linkers from Table 2, and a light chain sequence from Table 3. Non-limiting examples are included in Tables 4-6 and Tables 15-91.

In some embodiments, the payload region comprises a nucleic acid sequence encoding a single heavy chain. As a non-limiting example, the heavy chain is an amino acid sequence or fragment thereof described in Table 3.

Shown in Table 3 are a listing of antibodies and their polynucleotides and/or polypeptides sequences. These sequences may be encoded by or included in the AAV particles of the present disclosure. Variants or fragments of the antibody sequences described in Table 3 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics. NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 3. CDRs of the antibodies encoded by the viral genomes of the present disclosure may be 50%, 60%, 70%, 80%, 90%, 95% identical to CDRs listed in or incorporated in the sequences of Table 3. Methods of determining CDRs are well known in the art and are described herein. Payload regions may encode antibody variants with one or more heavy chain variable domain (V_{H}) or light chain variable domain (V_{L}) derived from the antibody sequences in Table 3. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any anti-tau antibodies, or tau-associated antibodies, not limited to those described in Table 3, including antibodies that are known in the art and/or antibodies that are commercially available. This may include fragments of such antibodies or antibodies that have been developed to comprise one or more of such fragments [e.g., variable domains or complementarity determining regions (CDRs)]. Anti-tau antibodies that may be encoded by payloads include, but are not limited to, AT8 (pSer²⁰²/pThr²⁰⁵; ThermoFisher, Waltham, Mass.; described in International Publication No. WO1995017429, the contents of which are herein incorporated in their entirety), AT100 (pThr²¹²/pSer²¹⁴; ThermoFisher, Waltham, Mass.; described in U.S. Pat. No. 6,121,003, the contents of which are herein incorporated in their entirety), AT180 (pThr²³¹; ThermoFisher, Waltham, Mass.; described in International Publication No. WO1995017429, the contents of which are herein incorporated by reference in their entirety), MC-1 (or MC1) (Tau^{2-18/312-342} conformational antibody; as described in international Publication WO199620218, the contents of which are herein incorporated by reference in their entirety), MC-6 (pSer²³⁵; described in U.S. Pat. No. 5,811,310, the contents of which are herein incorporated in their entirety). TG-3 (pThr²³¹; described in Jicha, G A et al., 1997 J Neurochem 69(5):2087-95, the contents of which are herein incorporated by reference in their entirety), CP13 (pSer²⁰²), CP27 (human Tau¹³⁰⁻¹⁵⁰), Tau12 (human Tau⁹⁻¹⁸; Abcam, Cambridge, Mass.), TG5 (Tau²²⁰⁻²⁴²; described in U.S. Pat. No. 5,811,310), DA9 (Tau¹⁰²⁻¹⁴⁰; described in U.S. Pat. No. 5,811,310), PHF1 (or PHF-1) (pSer³⁹⁶/pSer⁴⁰⁴; described in International Publication WO199620218), Alz50 (Tau⁷⁻⁹ and Tau³¹²⁻³⁴² conformational epitope; described in U.S. Pat. No. 5,811,310 and Carmel, G et al 1996 J Biol Chem 271(51):32780-32795 and Jicha, G A et al, 1997 J Neurosci Res 48(2):128-132, the contents of each of which are herein incorporated by reference in their entirety). Tau-1 (de-phosphorylated Ser¹⁹⁵/Ser¹⁹⁸/Ser¹⁹⁹/Ser²⁰²; ThermoFisher, Waltham, Mass.), Tau46 (Tau⁴⁰⁴⁻⁴⁴¹; Abcam, Cambridge, Mass.), pS199 (ThermoFisher, Waltham, Mass.), pT205, pS396 (ThermoFisher, Waltham, Mass.; described in U.S. Pat. No. 8,647,631, the contents of which are herein incorporated by reference in their entirety), pS404 (ThermoFisher, Waltham, Mass.; described in U.S. Pat. No. 8,647,631, the contents of which are herein incorporated by reference in their entirety), pS422 (ThermoFisher, Waltham, Mass.), A0024 (hTau²⁴³⁻⁴⁴¹; Dako, Glostrup, Denmark), HT7 (hTau¹⁵⁹⁻¹⁶³; ThermoFisher, Waltham, Mass.), Tau2 (hTau⁵²⁻⁶⁸; Abcam, Cambridge, Mass.), AD2 (pSer³⁹⁶/pSer⁴⁰⁴; Bio-Rad Laboratories, Hercules, Calif.), AT120 (hTau²¹⁶⁻²²⁴; described in U.S. Pat. No. 5,843,779, the contents of which are herein incorporated by reference in their entirety), AT270 (pThr¹⁸¹; ThermoFisher, Waltham, Mass.), 12E8 (pSer²⁶² and/or Ser³⁵⁶), K9JA (hTau²⁴³⁻⁴⁴¹; Dako, Caprinteria, Calif.), TauC3 (hTau Asp441; Santa Cruz Biotechnology, Dallas, Tex.; described in United States Patent Publication US20120244174 and Gamblin, T C et al 2003 PNAS 100(17):10032-7, the contents of each of which are herein incorporated by reference in their entirety). 4E6G7 (pSer³⁹⁶/pSer⁴⁰⁴; described in United States Patent Publication No. US2010316564 and Congdon, E. E. et al., 2016. Molecular Neurodegeneration August 30; 11(1):62, the contents of which are herein incorporated by reference in their entirety), 6B2 and variants thereof, described in International Patent Publication WO2016007414, the contents of which are herein incorporated by reference in their entirety. RZ3 (pThr²³¹), PG5 (pSer⁴⁰⁹), BT2 (pS^{199/202}), DA31 (Tau¹⁵⁰⁻¹⁹⁰), CP9 (pThr²³¹) Ta1505 (phospho site between Tau⁴¹⁰⁻⁴²¹, particularly pSer⁴¹³ as described in United States Patent Publication US20150183854 and Umeda, T. et al., 2015. Ann Clin Trans Neurol 2(3): 241-255, the contents of each of which are herein incorporated by reference in their entirety), PHF-6 (pThr²³¹, as described in Hoffman R et al., 1997. Biochemistry 36:8114-8124, the contents of which are herein incorporated by reference in their entirety), PHF-13 (pSer³⁹⁶, as described in Hoffman R et al., 1997. Biochemistry 36; 8114-8124), 16B5 (Tau²⁵⁻⁴⁶, as described in United States Publication US20160031976, the contents of which are herein incorporated by reference in their entirety), DC8E8 (as described in United States Patent Publication US20150050215, the contents of which are herein incorporated by reference in their entirety), PT1 or PT3 (as described in U.S. Pat. No. 9,371,376, the contents of which are herein incorporated by reference in their entirety), 4G11 (Tau⁵⁴⁻⁶⁴, as described in International Publication WO2016137950, the contents of which are herein incorporated by reference in their entirety). 1A6 (Tau⁷⁻¹⁷ and Tau²¹⁵⁻²²⁰, as described in International Publication WO2016137950), Tau15 or Tau81 (as described in International Publication WO2016055941, the contents of which are herein incorporated by reference in their entirety), TOC-1 (dimerized or aggregated tau, as described in International Publication WO2012149365, the contents of which are herein incorporated by reference in their entirety), pS4041gG2a/k (Neotope Biosciences, South San Francisco, Calif.; as described in Ittner et al., 2015. Neurochemistry 132:135-145, the contents of which are herein incorporated by reference in their entirety), TOMA (tau oligomer monoclonal antibody; as described in U.S. Pat. Nos. 8,778,343 and 9,125,846, International Publications WO2012051498 and WO2011026031, or United States Publication Nos. US20150004169 and US20150322143, and Castillo-Carranza, D L et al., 2014 J Neurosci 34(12)4260-72, the contents of each of which are herein incorporated by reference in their entirety), TTC-99 (oligomeric tau), BMS-986168 (as described in United States Patent Publication US2014294831, International Publication WO2015081085 and U.S. Pat. No. 8,980,271, the contents of which are herein incorporated by reference in their entirety), 3H3 (pan-amyloid epitope; described in Levites, Y et al 2015 J Neurosci 35(16)6265-76, the contents of which are herein incorporated by reference in their entirety), cis-pT231 (described in International Publications WO2012149334 and WO2011056561, the contents of which are herein incorporated by reference in their entirety), CP-3 (pSer²¹⁴; described in Jicha et al 1999 J Neurosci 19(17):7486-94, the contents of which are herein incorporated by reference in their entirety). TNT1 (Tau²⁻¹⁸; as described in United States Patent Publication 20160031978, the contents of which are herein incorporated by reference in their entirety), Tau-nY29 (nTyr²⁹; described in Reynolds M R, et al., 2006 J Neurosci 26(42):10636-45, the contents of which are herein incorporated by reference in their entirety), Tau-nY197 (nTyr¹⁹⁷; described in Reyes, J F et al., 2012 Acta Neuropathol 123(1):119-32, the contents of which are herein incorporated by reference in their entirety), Tau-nY394 (nTyr³⁹⁴; described in Reyes, J F et al 2012), 4E4 (Tau³³⁷⁻³⁴³ Tau³⁸⁷⁻³⁹⁷; described in International Publication WO2012049570 and United States Patent Publication US20150252102, the contents of each of which are herein incorporated by reference in their entirety), ADx210 (described in United States Patent Publication US20140161875, the contents of which are herein incorporated by reference in their entirety), ADx215 (described in United States Patent Publication US20140161875), ADx202 (as described in International Publication WO2015004163, the contents of which are herein incorporated by reference in their entirety), AP422 (pSer⁴²²; described in Hasegawa, M et al 1996 FEBS Lett 384:25-30, the contents of which are herein incorporated by reference in their entirety), Tau5 (Tau²¹⁰⁻²⁴¹), RTA2 (Tau²⁷³⁻²⁸³), RTAC (Tau⁴²⁶⁻⁴⁴¹), RTA1 (Tau²⁵⁷⁻²⁷⁴), T46 (Tau³⁹⁴⁻⁴³²), T49, MIGT4, O.BG.15, 525, 3-39, 4F1, MapTau (Tau⁹⁴⁻¹⁰⁸; SMI Covance), T1, HYB33801 (Tau⁵⁻¹²), Tau13 (Tau²⁻¹⁸), B11E8, 5J20 (14-3-3 tau), DC25 (Tau³⁴⁷⁻³⁵³), DC39N1 (Tau⁴⁵⁻⁷³), DC-11 (Tau³²¹⁻³⁹¹; described in U.S. Pat. No. 7,746,180, the contents of which are herein incorporated by reference in their entirety), DC39 (Tau⁴⁰¹⁻⁴¹¹), DC4R, n847 (nitrated tau), SPM452, T14. 1E1/A6 (Tau²⁷⁵⁻²⁹¹), 5E2, 8E6/C11 (Tau²⁰⁹⁻²²⁴), 2E12 (pT231), NFT200, 248E5 (Tau³⁻²¹⁴), IG2 (Thr¹⁷⁵, Thr¹⁸¹, Thr²³¹; as described in International Publication WO2016041553, the contents of which are herein incorporated by reference in their entirety), YP3 (as described in WO2007019273, the contents of which are herein incorporated by reference in their entirety), YP4 (as described in WO2007019273) and 14-3-3 Tau (pSer 14-3-3 binding motif; Abcam, Cambridge, Mass.). Further, anti-tau antibodies may be any of those listed in the antibody section of Alzforum.org or at the Antibody Resource Page.com, the contents of each of which are herein incorporated by reference in their entirety. Further, anti-tau antibodies may be any commercially available anti-tau antibody. Additional antibodies may include any of those taught in Petry, F. R. et al., 2014. PLoS One 9(5): e94251, the contents of which are herein incorporated by reference in their entirety. In one example, such antibodies may include any of those described in Jicha, G. A. et al., 1997. Journal of Neuroscience Research 48:128-132, the contents of which are herein incorporated by reference in their entirety. One such antibody, MC-1 (or MC1), recognizes distinct conformations of tau that are associated with neurological disease.

In some embodiments, the AAV particles may have a payload region comprising any of the anti-tau antibodies as described in International Publication WO2017189963, the contents of which are herein incorporated by reference in their entirety. As a non-limiting example, the payload region may comprise one or more of the anti-tau antibodies as described in Table 3 of International Publication WO2017189963. In some embodiments, the payload region encodes one or more anti-tau antibodies selected from SEQ ID NO: 2948-4269 as described in WO2017189963.

In some embodiments, payloads may encode anti-tau antibodies (or fragments thereof) taught in United States Publication No. US2014294831, the contents of which are herein incorporated by reference in their entirety. Such antibodies may include IPN001 and/or IPN002 antibodies or fragments of such antibodies. In some cases, variable domains of IPN002 as presented in FIGS. 2A and 2B of US2014294831 may be used (e.g., incorporated into another antibody). In some cases, CDR regions of IPN002 as underlined in FIGS. 2A and 2B may be used (e.g., incorporated into another antibody or used to prepare humanized versions of IPN002). In some cases, anti-tau antibodies may include any of the IPN001 or IPN002 antibody variants taught in US2014294831 (e.g., in FIGS. 9-16 of that publication). In some embodiments, this antibody is also referred to as BMS-986168.

In some cases, payloads may encode anti-tau antibodies (or fragments thereof) taught in Otvos, L. et al., 1994. J Neurosci. Res 39(6):669-73, the contents of which are herein incorporated by reference in their entirety. Such antibodies may include monoclonal antibody PHF-1 or fragments thereof. The PHF-1 antibody binds to tau paired helical filaments, a pathological conformation of tau, found in certain neurological disorders, including Alzheimer's disease. Further, antibody affinity is increased when either serine 396 or serine 404 of tau is phosphorylated and even further increased when both are phosphorylated.

In some embodiments, payloads may encode anti-tau antibodies (or fragments thereof) taught in U.S. Pat. No. 5,811,310, the contents of which are herein incorporated by reference in their entirety. Such embodiments may include monoclonal antibodies PHF-1 or MC1 or fragments thereof. MC1 is a conformational antibody binding to the epitopes presented in Jicha, G. A., et al., 1997. J Neurosci Res 48(128-132).

In some embodiments, payloads may encode anti-tau antibodies (or fragments thereof) taught in International Publication Number WO2015035190, the contents of which are herein incorporated by reference in their entirety. Such embodiments may include, but are not limited to, antibodies PHF-1 or MC1 or fragments thereof. Viral genomes of the AAV particles of the present disclosure may comprise or encode any of SEQ ID NO: 1-6 of WO2015035190.

In some embodiments, viral genomes may encode anti-tau antibody MC1 scFv as described in Vitale et al 2018, (Acta Neuropath Commun. 6:82) the contents of which are herein incorporated by reference in their entirety.

In some embodiments, viral genomes may encode anti-tau antibody MC1 as described in International Publication WO2016137811, the contents of which are herein incorporated by reference in their entirety.

Anti-tau antibodies (or fragments thereof) encoded by viral genomes may include antibodies that bind to one or more of the epitopes presented in Otvos, L. et al., 1994. J Neurosci. Res 39(6):669-73 (e.g., any of those presented in Table 1 of that publication).

In some embodiments, payloads may encode anti-tau antibodies (or fragments thereof) taught in U.S. Pat. No. 7,746,180, the contents of which are herein incorporated by reference in their entirety. Such embodiments may include antibody DC-11 or fragments thereof.

In some embodiments, the antibodies encoded by the viral genomes of the present disclosure may target any of the antigenic regions or epitopes described in United States Patent Publication No US2008050383 or US20100316564, the contents of which are herein incorporated by reference in their entirety. In some embodiments, the antibody targets pS396/pS404. Such embodiments may include antibody 4E6 and/or variants or fragments thereof. The affinity of antibody 4E6 for soluble PHF and its ability to reduce soluble phospho tau has been described in Congdon, E. E. et al., 2016. Molecular Neurodegeneration August 30; 11(1):62, the contents of which are herein incorporated by reference in their entirety.

In some embodiments, the antibodies encoded by the viral genomes of the present disclosure may target any of the antigenic regions or epitopes described in International Patent Publication WO1998022120, the contents of which are herein incorporated by reference in their entirety. In some embodiments, the antibody may be PHF-6 (pT231), or fragments or variants thereof. In another embodiment, the antibody may be PHF-13 (pS396), or a fragment of variant thereof. These antibodies are further described in Hoffman et al., 1997. Biochemistry 36: 8114-8124, the contents of which are herein incorporated by reference in their entirety.

In some embodiments, the antibodies encoded by the viral genomes of the present disclosure may target any of the antigenic regions or epitopes described in International Publication WO2016126993, the contents of which are herein incorporated by reference in their entirety. The antibodies may be derived from any of the tau epitopes described in Table A of WO2016126993. In some embodiments, the antibody of the present disclosure may comprise any of the sequences listed in Table B or Table 1 of WO2016126993.

In some embodiments, the antibodies encoded by the viral genomes of the present disclosure may target any of the antigenic regions or epitopes described in United States Patent Publication US20120244174, the contents of which are herein incorporated by reference in their entirety. In some embodiments, the antibody may bind to caspase-cleaved tau. In some embodiments, the epitope for antibodies targeting caspase cleaved tau is aspartic acid 421. In another embodiment, the epitope for antibodies targeting caspase cleaved tau may be the C-terminus after glutamic residue Glu391. In yet another embodiment, the epitope for antibodies targeting caspase cleaved tau may be at the N-terminus at aspartic acid residue 13. In another embodiment, the antibody may be TauC3.

In some embodiments, the antibodies encoded by the viral genomes of the present disclosure may target any of the antigenic regions or epitopes described in United States Patent Publication US20160031978, the contents of which are herein incorporated by reference in their entirety. In some embodiments, the antibody may bind to tau N-terminal residues associated with the PP1/GSK3 signaling cascade. In some embodiments, the antibody may be TNT1.

In some embodiments, the antibodies encoded by the viral genomes of the present disclosure may be any of those described in d'Abramo, C et al., 2015. PLOS One 10(8):e0135774, the contents of which are herein incorporated by reference in their entirety. In some embodiments, the antibody may be CP13 (pS202), or a fragment or variant thereof. In another embodiment, the antibody may be RZ3 (pT231), or a fragment or variant thereof. In another embodiment, the antibody may be PG5 (pS409), or a fragment or variant thereof.

Anti-tau antibodies or fragments thereof encoded by the viral genomes of the present disclosure may target tau in any antigenic form. As non-limiting examples, antigenic tau may be an unphosphorylated or unmodified tau protein, a phosphorylated or otherwise post-translationally modified tau protein (0-GlnAcylated, or nitrosylated), an oligomeric species of tau protein, a soluble species of tau protein, an insoluble species of tau protein, a conformationally abnormal species of tau protein, a neuropathological form of tau protein and/or a neurofibrillary tangle or a precursor thereof.

Anti-tau antibodies or fragments thereof encoded by the viral genomes, may target any antigenic region or epitope along the full length of any of the six human tau protein isoforms. As non-limiting examples, the targeted antigenic peptides of the tau protein may be any of the following phosphorylated sites pT50, pS396, pS396-pS404, pS404, pS396-pS404-pS422, pS409, pS413, pS422, pS198, pS199, pS199-pS202, pS202, pT205, pT212, pS214, pT212-pS214, pT181, pT231, cis-pT231, pS235, pS238, pT245, pS262, pY310, pY394, pS324, pS356, pTau¹⁷⁷⁻¹⁸⁷, pY18, pS610, pS622, nitrosylated tau (nY18, nY29), methylated tau (di-meK281, dimeK311), O-GlnAcylated tau at S400, any of the following acetylated sites acK174, acK274, acK280, acK281 and/or any combination thereof. Acetylated tau proteins and associated antigenic peptides are described in Min et al., 2010, Neuron., 67, 953-966, Min et al., 2015, Nature Medicine., 10, 1154-1162, Cohen et al., 2011, Nature Communications., 2, 252, Gorsky et al., 2016, Scientific Report., 6, 22685, Tracy et al., 2016, Neuron., 90, 245-260, the contents of each of which are herein incorporated by reference in their entirety. Phosphorylated tau proteins and associated antigenic peptides are described in Asuni et al., 2007. J Neurosci., 27, 9115-9129. Boutajangout et al., 2010. J Neurosci., 30, 16559-16566, Boutajangout et al., 2011, J Neurochem., 118, 658-667. Chai et al., 2011, J Biol Chem., 286, 34457-34467, Gu et al., 2011, J Biol Chem., 288, 33081-33095, Sankaranarayanan et al., 2015, PloS One, 10, e0125614, Ittner et al., 2015, J Neurochem., 132, 135-145, D'Abramo et al., 2016, Neurobiol Aging., 37, 58-65, Collin et al., 2014, Brain., 137, 2834-2846, Kondo et al., 2015, Nature., 523, 431-436, the contents of each of which are herein incorporated by reference in their entirety.

In some embodiments, the antibody encoded by the viral genomes of the present disclosure may be a pS409 targeting antibody as described in Lee et al., 2016, Cell Reports, 16, 1690-1700, or International Patent Publication WO2013151762, the contents of each of which am herein incorporated by reference in their entirety. In some embodiments, this antibody may be RG6100 or R071057 or variants or fragments thereof.

In some embodiments, the antibody encoded by the viral genomes of the present disclosure may be a pS413 targeting antibody as described in Umeda et al., 2015, Ann Clin Trans Neurol., 2(3), 241-255 or International Patent Publication WO2013180238, the contents of each of which are herein incorporated by reference in their entirety. In some embodiments, the antibody is Ta1505 or variants or fragments thereof.

In some embodiments, the antibody encoded by the viral genomes of the present disclosure may target a tau epitope with amino acid residues 210-275, more specifically pS238 and/or pT245, as described in International Publication WO2011053565, the contents of which are herein incorporated by reference in their entirety.

In some embodiments, the CDRs of an antibody encoded by the viral genomes of the present disclosure may be any of those listed in or incorporated in the antibody sequences of Table 3. In some embodiments, the CDRs may be any of those described in International Publication WO2015122922, the contents of which are herein incorporated by reference in their entirety. In some embodiments, a CDR may be any of those chosen from the group of SEQ ID NO: 41, 49, or 57 of WO2015122922. Further a CDR of an antibody encoded by the viral genomes of the present disclosure may have 50%, 60%, 70%, 80%, 90%, or 95% identity to SEQ ID NO: 41, 49, or 57 of WO2015122922.

In some embodiments, the antibodies encoded by the viral genomes of the present disclosure may be any of those described in International Publication WO2016097315, the contents of which are herein incorporated by reference in their entirety. In some embodiments, the antibody may have an amino acid sequence as shown by SEQ ID NO: 2, 11, 20, 29, 38, 47, 56, 65, 74, 83, 92, 101, 110, 119, 128, 137, 146, 155, 164, 173, 182, 191, 209, 218, 226, or 227 of WO2016097315.

In some embodiments, the antibodies encoded by the viral genomes of the present disclosure may be a multispecific blood brain barrier receptor antibody that also targets tau, as described in International Publication WO2016094566, the contents of which are herein incorporated by reference in their entirety. In some embodiments, the antibody may have a sequence as shown by SEQ ID NO: 1, 2, 17, 18, 33, 34, 49, 50, 65, 66, 81, 82, 9-16, 25-32, 41-48, 57-64, 73-80, 89-96 of WO2016094566.

In some embodiments, the antibodies (or fragments thereof) encoded by the viral genomes of the present disclosure may be any of those taught in U.S. Pat. Nos. 8,778,343 and 9,125,846, International Publications WO2012051498 and WO2011026031, or United States Publication Nos. US20150004169 and US20150322143, the contents of each of which are herein incorporated by reference in their entirety. Such antibodies may include those that bind to oligomeric species of tau. Further, such an antibody may be referred to as TOMA (tau oligomer monoclonal antibody), as described in Castillo-Carranza et at (Castillo-Carranza, D L et al., 2014 J Neurosci 34(12)4260-72) the contents of which are herein incorporated by reference in their entirety. In some embodiments, the antibody that binds oligomeric tau may be TTC-99.

In some embodiments, the antibodies (or fragments thereof) encoded by the viral genomes of the present disclosure may be any of those taught in International Publications WO2014059442, the contents of which are herein incorporated by reference in their entirety. Such antibodies may include those that bind to oligomeric species of tau.

In some embodiments, the antibodies (or fragments thereof) encoded by the viral genomes of the present disclosure may be any of those taught in the International Publications WO2014008404 and WO2016126993, United States Patent Publication US20150183855, Yanamandra, K et al., 2013 Neuron 80(2):402-14 and Yanamandra, K et al 2015 Ann Clin Transl Neurol 2(3):278-88, the contents of each of which are herein incorporated by reference in their entirety. Such antibodies may block tau seeding. Non-limiting examples of antibodies described in these publications include HJ8.1.1, HJ8.1.2, HJ8.2, HJ8.3, HJ8.4, HJ8.5, HJ8.7, HJ8.8, HJ9.1, HJ9.2, HJ9.3, HJ9.4, HJ9.5, and variants thereof. Non-limiting examples of targeted epitopes of tau may include amino acids 22-34, 385-391, 405-411, 3-6, 118-122, 386-401, 7-13, and/or 272-281 of human tau.

In some embodiments, the antibodies (or fragments thereof) encoded by the viral genomes of the present disclosure may be any of those taught in the International Publications WO2002062851, the contents of which are herein incorporated by reference in their entirety.

In some embodiments, the antibodies (or fragments thereof) encoded by the viral genomes of the present disclosure may be as described in Bright, J et al., 2015 Neurobiol of Aging 36:693-709; Pedersen, J T and Sigurdsson E M, 2015 Trends Mol Med 21(6):394-402; Levites, Y et al 2015 J Neurosci 35(16)6265-76; Jicha et al 1999 J Neurosci 19(17):7486-94; Reyes J F et al., 2012 Acta Neuropathol 123(1):119-32; Reynolds M R, et al., 2006 J Neurosci 26(42):10636-45; Gamblin, T C et al 2003 PNAS 100(17):10032-7; Castillo-Carranza, D L et al., 2014 J Neurosci 34(12)4260-72; Walls, K C et al., 2014 Neurosci Lett 575:96-100; Yanamandra, K et al., 2013 Neuron 80(2):402-14; Yanamandra, K et al 2015 Ann Clin Transl Neurol 2(3):278-88: Allen B. et al., 2002 J Neurosci 22(21):9340-51: Gotz, J et al., 2010 Biochem Biophys Acta 1802(10):860-71; Hasegawa, M et al 1996 FEBS Lett 384:25-30; Carmel, G et al 1996 J Biol Chem 271(51):32780-32795; Jicha, G A et al, 1997 J Neurosci Res 48(2):128-132; Jicha, G A et al., 1997 J Neurochem 69(5):2087-95; the contents of each of which are herein incorporated by reference in their entirety.

Anti-tau antibodies or fragments thereof encoded by the viral genomes of the present disclosure may be any commercially available anti-tau antibody known in the art or developed by a person with skill in the art. Non-limiting examples of commercially available anti-tau antibodies include EPR2396(2) (pThr⁵⁰; Abeam, Cambridge, Mass.), 5H911 (pThr¹⁸³; ThermoFisher, Waltham, Mass.), M7004D06 (pThr¹⁸¹; BioLegend, San Diego, Calif.), 1E7 (pThr¹⁸¹; EMD Millipore, Billerica, Mass.), EPR2400 (pSer¹⁹⁸; Abcam, Cambridge, Mass.), EPR2401Y (pSer¹⁹⁹; Abcam, Cambridge, Mass.), 2H23L4 (pSer¹⁹⁹; ThermoFisher, Waltham, Mass.), EPR2402 (pSer²⁰²; Abcam, Cambridge, Mass.), 10F8 (pSer²⁰²; Abcam, Cambridge, Mass.), EPR2403(2) (pThr²⁰⁵; Abcam, Cambridge, Mass.), EPR1884(2) (pSer²¹⁴; Abcam, Cambridge, Mass.), EPR2488 (pThr²³¹; Abcam, Cambridge, Mass.), 1H6L6 (pThr²³¹; ThermoFisher, Waltham, Mass.), 3G3 (pThr²³¹, pSer²³⁵; Abeam, Cambridge, Mass.), EPR2452 (pSer²³⁵; Abcam, Cambridge, Mass.), 12G10 (pSer²³⁹; Abcam, Cambridge, Mass.), EPR2454 (pSer²⁶²; Abcam, Cambridge, Mass.), EPR2457(2) (pSer³²⁴; Abcam, Cambridge, Mass.), EPR2603 (pSer³⁵⁶; Abcam, Cambridge, Mass.), EPR2731 (pSer³⁹⁶; Abcam, Cambridge, Mass.), EPR2605 (pSer⁴⁰⁴; Abcam, Cambridge, Mass.), EPR2866 (pSer⁴²²; Abcam, Cambridge, Mass.), 1A4 (pTau¹⁷⁷⁻¹⁸⁷; Origene, Rockville, Md.), 7G9 (pTau¹⁷⁷⁻⁸⁷; Origene, Rockville, Md.), 9B4 (pTau¹⁷⁷⁻¹⁸⁷; Origene, Rockville, Md.), 2A4 (pTau¹⁷⁷⁻¹⁸⁷; Origene, Rockville, Md.), 9G3 (pTyr¹⁸; NovusBio, Littleton, Colo.), EPR2455(2) (pSer⁶¹⁰; Abcam, Cambridge, Mass.), EP2456Y (pSer⁶²²; Abcam, Cambridge, Mass.; EMD Millipore, Billerica, Mass.), SMI 51 (PHF Tau⁹⁵⁻¹⁰⁸; BioLegend, San Diego, Calif.), TOMA-1 (Oligomeric Tau; EMD Millipore, Billerica, Mass.), Tau-nY18 (nTyr¹⁸; Origene, Rockville, Md.; BioLegend, San Diego, Calif.; EMD Millipore, Billerica, Mass.), Tau-nY29 (nTyr²⁹; BioLegend, San Diego, Calif.; EMD Millipore, Billerica, Mass.; Abcam, Cambridge, Mass.), 1C9.G6 (di-methyl-Lys²⁸¹; BioLegend, San Diego, Calif.), 7G5.F4 (di-methyl-Lys³¹¹; BioLegend, San Diego, Calif.), TNT-1 (Tau²⁻¹⁸; EMD Millipore, Billerica, Mass.), TNT-2 (Tau²⁻¹⁸; EMD Millipore. Billerica, Mass.). 7B8 (Tau⁵⁻¹²; Abcam, Cambridge, Mass.), Tau-13 (Tau²⁰⁻³⁵; BioLegend, San Diego, Calif.), 1-100 (Tau¹⁻¹⁰⁰; BioLegend, San Diego, Calif.), 2G9.F10 (Tau¹⁵⁷⁻¹⁶⁸; BioLegend, San Diego, Calif.; Origene, Rockville, Md.), 39E10 (Tau¹⁸⁹⁻¹⁹⁵; BioLegend, San Diego, Calif.; Origene, Rockville, Md.), 77E9 (Tau¹⁸⁵⁻¹⁹⁵; BioLegend, San Diego, Calif.; Origene, Rockville, Md.), AT8 (pSer²⁰², pThr²⁰⁵; ThermoFisher, Waltham, Mass.), AT100 (pThr²²¹, pSer²¹⁴; ThermoFisher, Waltham, Mass.), PHF-6 (pThr²³¹; NovusBio, Littleton, Colo.; EMD Millipore, Billerica, Mass.; BioLegend, San Diego, Calif.; ThermoFisher, Waltham, Mass.), AT180 (pThr²³¹; ThermoFisher, Waltham, Mass.), AT270 (pThr¹⁸¹; ThermoFisher, Waltham, Mass.), PHF-13 (pSer³⁹⁶; ThermoFisher, Waltham, Mass.; BioLegend, San Diego, Calif.), TauC3 (Asp⁴²¹; BioLegend, San Diego. Calif.; EMD Millipore, Billerica, Mass.; ThermoFisher, Waltham, Mass.), Tau12 (Tau⁶⁻¹⁸; BioLegend, San Diego, Calif.; EMD Millipore, Billerica, Mass.), Tau5 (Tau²¹⁰⁻²⁴¹; BioLegend, San Diego, Calif.; EMD Millipore, Billerica, Mass.; Abcam, Cambridge Mass.; ThermoFisher, Waltham, Mass.), HT7 (Tau¹⁵⁹⁻¹⁶³; ThermoFisher, Waltham, Mass.), 77G7 (Tau³¹⁶⁻³⁵⁵; BioLegend, San Diego, Calif.), Tau46 (Tau⁴⁰⁴⁻⁴⁴¹; BioLegend, San Diego, Calif.; NovusBio, Littleton, Colo.; Abcam, Cambridge, Mass.), UMAB239 (Tau⁶²³⁻⁷⁵⁸; Origene, Rockville, Md.), OTI16G3 (Tau⁶²³⁻⁷⁵⁸; Origene, Rockville, Md.), OTI13E11 (Tau⁶²³⁻⁸⁰⁰; Origene, Rockville, Md.), OTI13B5 (Tau⁶²³⁻⁷⁵⁸; Origene, Rockville, Md.), E178 (Tau⁷⁰⁰⁻⁸⁰⁰; Abcam, Cambridge, Mass.), SP70 (N-terminal Tau; Origene, Rockville, Md.; NovusBio, Littleton, Colo.; ThermoFisher, Waltham, Mass.; Abcam, Cambridge, Mass.), C45 (N-terminal Tau; Origene, Rockville, Md.), Tau7 (C-terminal Tau; EMD Millipore, Billerica, Mass.), S.125.0 (C-terminal Tau; ThermoFisher, Waltham, Mass.), 8E6/C11 (Three-repeat Tau²⁰⁹⁻²²⁴; EMD Millipore, Billerica, Mass.), 1E1/A6 (Four-repeat Tau²⁷⁵⁻²⁹¹; EMD Millipore, Billerica, Mass.), 7D12.1 (Four-repeat Tau²⁷⁵⁻²⁹¹; EMD Millipore. Billerica, Mass.), 5C7 (Four-repeat Tau²⁶⁷⁻²⁷⁸; BioLegend, San Diego, Calif.; Origene, Rockville, Md.), 5F9 (Four-repeat Tau²⁷⁵⁻²⁹¹; BioLegend, San Diego, Calif.; Origene, Rockville, Md.), 3H6.H7 (0N Tau³⁹⁻⁵⁰; BioLegend, San Diego, Calif.; Origene, Rockville, Md.), 4H5.B9 (1N Tau⁶⁸⁻⁷⁹; BioLegend, San Diego, Calif.; Origene, Rockville, Md.), 71C11 (2N Tau; BioLegend, San Diego, Calif.), PC1C6 (unphosphorylated tau; EMD Millipore, Billerica, Mass.), Tau2 (BioLegend, San Diego, Calif.; Origene, Rockville, Md.; EMD Millipore, Billerica, Mass.), 2E9 (Origene, Rockville, Md.; NovusBio, Littleton, Colo.), 4F1 (Origene, Rockville, Md.; NovusBio, Littleton, Colo.), 5B10 (NovusBio, Littleton, Colo.); 5E2 (EMD Millipore, Billerica, Mass.), Tau-93 (Origene, Rockville, Md.), T14 (ThermoFisher, Waltham, Mass.), T46 (ThermoFisher, Waltham, Mass.), BT2 (ThermoFisher, Waltham, Mass.) and/or variants or derivates thereof.

In some embodiments, the antibodies encoded by the viral genomes of the present disclosure may be multispecific antibodies for transferrin receptor and a brain antigen, wherein the brain antigen may be tau, as described in International Publication WO2016081643, the contents of which are herein incorporated by reference in their entirety. In some embodiments, the antibody may have a sequence as given by SEQ ID NO: 160 or 161 of WO2016081643.

In some embodiments, the antibodies encoded by the viral genomes of the present disclosure are any of those described in U.S. Pat. Nos. 8,871,447, 8,420,613, International Publication No. WO2014193935, WO2010011999, or in United States Publication Nos. US20110250217, US20110020237, US20100316590, or US20120225864, the contents of each of which are herein incorporated by reference in their entirety. In some embodiments, the antibody recognizes a misfolded, amyloidogenic or aggregating protein.

In some embodiments, the viral genome of the AAV particle of the present disclosure encodes anti-tau antibody PT3, or a fragment or variant thereof.

In some embodiments, the viral genome of the AAV particle of the present disclosure encodes anti-tau antibody AT8, or a fragment or variant thereof.

In some embodiments, the viral genome of the AAV particle of the present disclosure encodes anti-tau antibody IPN002, or a fragment or variant thereof.

In some embodiments, the viral genome of the AAV particle of the present disclosure encodes anti-tau antibody MC1, or a fragment or variant thereof.

In some embodiments, the viral genome of the AAV particle of the present disclosure encodes anti-tau antibody PHF1, or a fragment or variant thereof.

In some embodiments, the viral genome of the AAV particle of the present disclosure encodes anti-tau antibody CP13, or a fragment or variant thereof.

In some embodiments, the viral genome of the AAV particle of the present disclosure encodes anti-tau antibody C10.2, or a fragment or variant thereof.

In some embodiments, the viral genome of the AAV particle of the present disclosure encodes anti-tau antibody PHF-13, or a fragment or variant thereof.

In some embodiments, the viral genome of the AAV particle of the present disclosure encodes anti-tau antibody PHF-6, or a fragment or variant thereof.

### The Nature of the Polypeptides and Variants

Antibodies encoded by payload regions of the viral genomes may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and preferably, they will be at least about 80/6, more preferably at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions, or deletions of amino acid residues that still maintain the properties of the parent polypeptide.

Sequence tags or amino acids, such as one or more lysines, can be added to the peptide sequences (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure include surface manifestations, local conformational shape, folds, loops, half-loops, domains, half-domains, sites, termini or any combination thereof.

As used herein when referring to proteins the term “surface manifestation” refers to a polypeptide-based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830: 1997).

As used herein when referring to proteins the term “half-loop” refers to a portion of an identified loop having at least half the number of amino acid residues as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7-amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid residues as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7-amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid-based embodiments is used synonymous with “amino acid residue” and “amino acid side chain”. A site represents a position within a peptide or polypeptide that may be modified, manipulated, altered, derivatized or varied within the polypeptide-based molecules of the present disclosure.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide-based molecules of the present disclosure may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a component of a molecule, any of several manipulations and/or modifications of these features may be performed by moving, swapping, inverting, deleting, randomizing, or duplicating. Furthermore, it is understood that manipulation of features may result in the same outcome as a modification to the molecules. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### AAV Production

The present disclosure provides methods for the generation of parvoviral particles, e.g. AAV particles, by viral genome replication in a viral replication cell.

In accordance with the disclosure, the viral genome comprising a payload region encoding an antibody, an antibody-based composition or fragment thereof, will be incorporated into the AAV particle produced in the viral replication cell. Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088. WO1999014354, WO1999015685, WO1999047691, WO2000055342. WO2000075353, and WO2001023597; Methods In Molecular Biology, ed. Richard. Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In some embodiments, the AAV particles are made using the methods described in WO2015191508, the contents of which are herein incorporated by reference in their entirety.

Viral replication cells commonly used for production of recombinant AAV viral vectors include but are not limited to 293 cells, COS cells, HeLa cells, KB cells, and other mammalian cell lines as described in U.S. Pat. Nos. U.S. Pat. Nos. 6,156,303, 5,387,484, 5,741,683, 5,691,176, and 5,688,676; U.S. patent publication No. 2002/0081721, and International Patent Publication Nos. WO 00/47757, WO 00/24916, and WO 96/17947, the contents of each of which are herein incorporated by reference in their entireties.

In some embodiments, the AAV particles of the present disclosure may be produced in insect cells (e.g., Sf9 cells).

In some embodiments, the AAV particles of the present disclosure may be produced using triple transfection.

In some embodiments, the AAV particles of the present disclosure may be produced in mammalian cells.

In some embodiments, the AAV particles of the present disclosure may be produced by triple transfection in mammalian cells.

In some embodiments, the AAV particles of the present disclosure may be produced by triple transfection in HEK293 cells.

The present disclosure provides a method for producing an AAV particle comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, 5) harvesting and purifying the viral particle comprising a parvoviral genome.

In some embodiments, the present disclosure provides a method for producing an AAV particle comprising the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

In some embodiments, the viral construct vector(s) used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In some embodiments, the viral construct vector(s) used for AAV production may contain a nucleotide sequence encoding the AAV rep proteins where the initiation codon of the AAV rep protein or proteins is a non-ATG. In some embodiments, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

In some embodiments, the viral genome of the AAV particle optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are selected from those described in International Application No. WO 96/23810; Heim et al., Current Biology 2:178-182 (1996); Heim et al., Proc. Natl. Acad. Sci. USA (1995); or Heim et al., Science 373:663-664 (1995); WO 96/30540, the contents of each of which are incorporated herein by reference in their entireties).

The AAV viral genomes encoding an anti-tau antibody payload described herein may be useful in the fields of human disease, veterinary applications and a variety of in vivo and in vitro settings. The AAV particles of the present disclosure may be useful in the field of medicine for the treatment, prophylaxis, palliation or amelioration of neurological diseases and/or disorders. In some embodiments, the AAV particles are used for the prevention and/or treatment of a tauopathy.

Various embodiments herein provide a pharmaceutical composition comprising the AAV particles described herein and a pharmaceutically acceptable excipient.

Various embodiments herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of intravenous, intracerebroventricular, intraparenchymal, intrathecal, subpial and intramuscular, or a combination thereof. Certain embodiments of the method provide that the subject is treated for a tauopathy and/or other neurological disorder. In one aspect of the method, a pathological feature of the tauopathy or other neurological disorder is alleviated and/or the progression of the tauopathy or other neurological disorder is halted, slowed, ameliorated or reversed.

Various embodiments herein describe a method of decreasing the level of soluble tau in the central nervous system of a subject in need thereof comprising administering to said subject an effective amount of the pharmaceutical composition described herein.

Also described herein are compositions, methods, processes, kits and devices for the design, preparation, manufacture and/or formulation of AAV particles. In some embodiments, payloads, such as but not limited to anti-tau antibodies, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of neurological disease, such as, but not limited to tauopathy.

### AAV Particles Comprising Anti-Tau Antibody Payloads

In some embodiments, the AAV particle comprises a viral genome with a payload region comprising one or more anti-tau antibody polynucleotide sequences. In such an embodiment, a viral genome encoding more than one polypeptide may be replicated and packaged into a viral particle. A target cell transduced with a viral particle comprising one or more anti-tau antibody polynucleotides may express the encoded antibody or antibodies in a single cell.

In some embodiments, the AAV particles are useful in the field of medicine for the treatment, prophylaxis, palliation or amelioration of neurological diseases and/or disorders.

Non-limiting examples of ITR to ITR sequences of AAV particles comprising a viral genome with a payload region comprising an anti-tau antibody polynucleotide sequence are described in Table 4-6.

TABLE 4
ITR to ITR Sequences of AAV particles comprising
anti-tau antibody polynucleotide sequences

| ITR to ITR | Sequence | ITR to ITR |
|---|---|---|
| Construct Name | Length (nt) | SEQ ID NO |
| TAU_ITR1 | 4450 | 1990 |
| TAU_ITR2 | 4471 | 1991 |
| TAU_ITR3 | 3983 | 1992 |
| TAU_ITR4 | 3962 | 1993 |
| TAU_ITR5 | 4041 | 1994 |
| TAU_ITR6 | 4020 | 1995 |
| TAU_ITR7 | 3899 | 1996 |
| TAU_ITR8 | 3878 | 1997 |
| TAU_ITR9 | 4459 | 1998 |
| TAU_ITR10 | 4438 | 1999 |
| TAU_ITR11 | 3971 | 2000 |
| TAU_ITR12 | 4029 | 2001 |
| TAU_ITR13 | 4008 | 2002 |
| TAU_ITR14 | 3887 | 2003 |
| TAU_ITR15 | 3866 | 2004 |
| TAU_ITR16 | 3932 | 2005 |
| TAU_ITR17 | 3932 | 2006 |
| TAU_ITR18 | 3932 | 2007 |
| TAU_ITR19 | 3932 | 2008 |
| TAU_ITR20 | 3932 | 2009 |
| TAU_ITR21 | 3932 | 2010 |
| TAU_ITR22 | 3932 | 2011 |
| TAU_ITR23 | 3932 | 2012 |
| TAU_ITR24 | 3932 | 2013 |
| TAU_ITR25 | 3932 | 2014 |
| TAU_ITR26 | 4309 | 2015 |
| TAU_ITR27 | 4309 | 2016 |
| TAU_ITR28 | 4309 | 2017 |
| TAU_ITR29 | 4309 | 2018 |
| TAU_ITR30 | 4309 | 2019 |
| TAU_ITR31 | 4309 | 2020 |
| TAU_ITR32 | 4309 | 2021 |
| TAU_ITR33 | 4309 | 2022 |
| TAU_ITR34 | 4309 | 2023 |
| TAU_ITR35 | 4309 | 2024 |
| TAU_ITR87 | 3950 | 2137 |
| TAU_ITR88 | 3962 | 2138 |
| TAU_ITR89 | 4450 | 2139 |
| TAU_ITR90 | 4020 | 2140 |
| TAU_ITR91 | 3878 | 2141 |
| TAU_ITR92 | 3950 | 2142 |
| TAU_ITR93 | 4438 | 2143 |
| TAU_ITR94 | 4008 | 2144 |
| TAU_ITR95 | 3866 | 2145 |
| TAU_ITR96 | 3941 | 2146 |
| TAU_ITR97 | 4429 | 2147 |
| TAU_ITR98 | 3999 | 2148 |
| TAU_ITR99 | 3857 | 2149 |
| TAU_ITR100 | 4420 | 2150 |
| TAU_ITR101 | 3990 | 2151 |
| TAU_ITR102 | 3848 | 2152 |
| TAU_ITR103 | 3971 | 2153 |
| TAU_ITR104 | 4459 | 2154 |
| TAU_ITR105 | 4029 | 2155 |
| TAU_ITR106 | 3887 | 2156 |
| TAU_ITR107 | 3938 | 2157 |
| TAU_ITR108 | 4447 | 2158 |
| TAU_ITR109 | 4017 | 2159 |
| TAU_ITR110 | 3875 | 2160 |
| TAU_ITR111 | 3950 | 2161 |
| TAU_ITR112 | 4438 | 2162 |
| TAU_ITR113 | 4008 | 2163 |
| TAU_ITR114 | 3866 | 2164 |
| TAU_ITR115 | 3938 | 2165 |
| TAU_ITR116 | 4426 | 2166 |
| TAU_ITR117 | 3996 | 2167 |
| TAU_ITR118 | 3854 | 2168 |
| TAU_ITR119 | 3950 | 2161 |
| TAU_ITR120 | 4438 | 2162 |
| TAU_ITR121 | 3455 | 2171 |
| TAU_ITR122 | 3498 | 2172 |
| TAU_ITR123 | 3282 | 2173 |
| TAU_ITR124 | 3300 | 2174 |
| TAU_ITR125 | 3943 | 2175 |
| TAU_ITR126 | 3988 | 2176 |
| TAU_ITR127 | 3772 | 2177 |
| TAU_ITR128 | 3790 | 2178 |
| TAU_ITR129 | 3324 | 2179 |
| TAU_ITR130 | 3814 | 2180 |
| TAU_ITR131 | 2567 | 2181 |
| TAU_ITR132 | 2582 | 2182 |
| TAU_ITR133 | 2597 | 2183 |
| TAU_ITR134 | 2612 | 2184 |
| TAU_ITR135 | 2627 | 2185 |
| TAU_ITR136 | 2642 | 2186 |
| TAU_ITR137 | 3055 | 2187 |
| TAU_ITR138 | 3070 | 2188 |
| TAU_ITR139 | 3085 | 2189 |
| TAU_ITR140 | 3100 | 2190 |
| TAU_ITR141 | 3115 | 2191 |
| TAU_ITR142 | 3130 | 2192 |
| TAU_ITR143 | 3160 | 2193 |
| TAU_ITR144 | 2569 | 2194 |
| TAU_ITR145 | 2584 | 2195 |
| TAU_ITR146 | 2599 | 2196 |
| TAU_ITR147 | 2614 | 2197 |
| TAU_ITR148 | 2627 | 2198 |
| TAU_ITR149 | 2642 | 2199 |
| TAU_ITR150 | 3055 | 2200 |
| TAU_ITR151 | 3070 | 2201 |
| TAU_ITR152 | 3084 | 2202 |
| TAU_ITR153 | 3100 | 2203 |
| TAU_ITR154 | 3115 | 2204 |
| TAU_ITR155 | 3130 | 2205 |
| TAU_ITR156 | 3097 | 2206 |
| TAU_ITR157 | 3082 | 2207 |
| TAU_ITR158 | 3097 | 2208 |
| TAU_ITR159 | 3112 | 2209 |
| TAU_ITR160 | 3097 | 2210 |
| TAU_ITR161 | 3082 | 2211 |
| TAU_ITR162 | 3097 | 2212 |
| TAU_ITR163 | 3112 | 2213 |
| TAU_ITR164 | 2652 | 2214 |
| TAU_ITR165 | 2667 | 2215 |
| TAU_ITR166 | 2730 | 2216 |
| TAU_ITR167 | 2510 | 2217 |
| TAU_ITR168 | 2525 | 2218 |
| TAU_ITR169 | 2540 | 2219 |
| TAU_ITR170 | 3091 | 2220 |
| TAU_ITR171 | 3082 | 2221 |
| TAU_ITR172 | 3103 | 2222 |
| TAU_ITR173 | 3109 | 2223 |
| TAU_ITR174 | 3115 | 2224 |
| TAU_ITR175 | 3121 | 2225 |
| TAU_ITR176 | 3097 | 2226 |
| TAU_ITR177 | 3124 | 2227 |
| TAU_ITR178 | 3109 | 2228 |
| TAU_ITR179 | 3157 | 2229 |
| TAU_ITR180 | 3760 | 2230 |
| TAU_ITR181 | 3781 | 2231 |
| TAU_ITR182 | 3778 | 2232 |
| TAU_ITR183 | 3748 | 2233 |
| TAU_ITR184 | 3769 | 2234 |
| TAU_ITR185 | 3766 | 2235 |
| TAU_ITR186 | 3760 | 2236 |
| TAU_ITR187 | 3778 | 2237 |
| TAU_ITR188 | 3188 | 2260 |
| TAU_ITR189 | 3330 | 2261 |
| TAU_ITR190 | 3976 | 2262 |
| TAU_ITR191 | 3404 | 2263 |
| TAU_ITR192 | 3546 | 2264 |
| TAU_ITR193 | 3997 | 2265 |
| TAU_ITR194 | 3425 | 2266 |
| TAU_ITR195 | 3567 | 2267 |
| TAU_ITR196 | 3088 | 2268 |
| TAU_ITR197 | 3558 | 2269 |
| TAU_ITR198 | 3360 | 2270 |
| TAU_ITR199 | 3384 | 2271 |
| TAU_ITR200 | 2625 | 2272 |
| TAU_ITR201 | 2655 | 2273 |
| TAU_ITR202 | 2685 | 2274 |
| TAU_ITR203 | 2730 | 2275 |
| TAU_ITR204 | 2625 | 2276 |
| TAU_ITR205 | 2655 | 2277 |
| TAU_ITR206 | 2685 | 2278 |
| TAU_ITR207 | 3416 | 2279 |
| TAU_ITR208 | 3218 | 2280 |
| TAU_ITR209 | 3242 | 2281 |
| TAU_ITR210 | 2483 | 2282 |
| TAU_ITR211 | 2513 | 2283 |
| TAU_ITR212 | 2543 | 2284 |
| TAU_ITR213 | 2588 | 2285 |
| TAU_ITR214 | 2483 | 2286 |
| TAU_ITR215 | 2513 | 2287 |
| TAU_ITR216 | 2543 | 2288 |
| TAU_ITR217 | 3967 | 2289 |
| TAU_ITR218 | 3769 | 2290 |
| TAU_ITR219 | 3792 | 2291 |
| TAU_ITR220 | 3751 | 2292 |
| TAU_ITR221 | 3058 | 2293 |
| TAU_ITR222 | 3088 | 2294 |
| TAU_ITR223 | 3118 | 2295 |
| TAU_ITR224 | 3163 | 2296 |
| TAU_ITR225 | 3537 | 2297 |
| TAU_ITR226 | 3339 | 2298 |
| TAU_ITR227 | 3362 | 2299 |
| TAU_ITR228 | 3321 | 2300 |
| TAU_ITR229 | 2628 | 2301 |
| TAU_ITR230 | 2658 | 2302 |
| TAU_ITR231 | 2688 | 2303 |
| TAU_ITR232 | 2733 | 2304 |
| TAU_ITR233 | 3395 | 2305 |
| TAU_ITR234 | 3197 | 2306 |
| TAU_ITR235 | 3220 | 2307 |
| TAU_ITR236 | 3179 | 2308 |
| TAU_ITR237 | 2486 | 2309 |
| TAU_ITR238 | 2516 | 2310 |
| TAU_ITR239 | 2546 | 2311 |
| TAU_ITR240 | 2591 | 2312 |

TABLE 5
ITR to ITR Sequences of AAV particles comprising
anti-tau antibody polynucleotide sequences

| ITR to ITR | Sequence | ITR to ITR |
|---|---|---|
| Construct Name | Length (nt) | SEQ ID NO |
| TAU_ITR36 | 4253 | 2025 |
| TAU_ITR37 | 4716 | 2026 |
| TAU_ITR38 | 4716 | 2027 |
| TAU_ITR39 | 4698 | 2028 |
| TAU_ITR40 | 4689 | 2029 |
| TAU_ITR41 | 4716 | 2030 |
| TAU_ITR42 | 4716 | 2031 |
| TAU_ITR43 | 4698 | 2032 |
| TAU_ITR44 | 4689 | 2033 |
| TAU_ITR45 | 4253 | 2034 |
| TAU_ITR46 | 2350 | 2035 |
| TAU_ITR47 | 2370 | 2036 |
| TAU_ITR48 | 2282 | 2037 |
| TAU_ITR49 | 2314 | 2038 |
| TAU_ITR50 | 2350 | 2039 |
| TAU_ITR51 | 2370 | 2040 |
| TAU_ITR52 | 2624 | 2041 |
| TAU_ITR53 | 3778 | 2042 |
| TAU_ITR54 | 2029 | 2043 |
| TAU_ITR55 | 2218 | 2044 |
| TAU_ITR56 | 3778 | 2045 |
| TAU_ITR57 | 2624 | 2046 |
| TAU_ITR58 | 2350 | 2047 |
| TAU_ITR59 | 2029 | 2048 |
| TAU_ITR60 | 2218 | 2049 |
| TAU_ITR61 | 4188 | 2050 |
| TAU_ITR62 | 3435 | 2051 |
| TAU_ITR63 | 4395 | 2052 |
| TAU_ITR64 | 3510 | 2053 |
| TAU_ITR65 | 4131 | 2054 |
| TAU_ITR66 | 3378 | 2055 |
| TAU_ITR67 | 4395 | 2056 |
| TAU_ITR68 | 3510 | 2057 |
| TAU_ITR69 | 4016 | 2058 |
| TAU_ITR70 | 4016 | 2059 |
| TAU_ITR71 | 3608 | 2060 |
| TAU_ITR241 | 3900 | 2313 |
| TAU_ITR242 | 4042 | 2314 |
| TAU_ITR243 | 3984 | 2315 |
| TAU_ITR244 | 4472 | 2316 |
| TAU_ITR245 | 2549 | 2317 |
| TAU_ITR246 | 2445 | 2318 |
| TAU_ITR247 | 2350 | 2319 |
| TAU_ITR248 | 4698 | 2320 |
| TAU_ITR249 | 4223 | 2321 |

TABLE 6
ITR to ITR Sequences of AAV particles comprising
anti-tau antibody polynucleotide sequences

| ITR to ITR | Sequence | ITR to ITR |
|---|---|---|
| Construct Name | Length (nt) | SEQ ID NO |
| TAU_ITR72 | 3619 | 2061 |
| TAU_ITR73 | 3610 | 2062 |
| TAU_ITR74 | 3607 | 2063 |
| TAU_ITR75 | 4158 | 2064 |
| TAU_ITR76 | 3598 | 2065 |
| TAU_ITR77 | 3622 | 2066 |
| TAU_ITR78 | 3619 | 2067 |
| TAU_ITR79 | 3607 | 2068 |
| TAU_ITR80 | 4158 | 2069 |
| TAU_ITR81 | 3607 | 2070 |
| TAU_ITR82 | 3598 | 2071 |
| TAU_ITR83 | 3610 | 2072 |
| TAU_ITR84 | 3610 | 2073 |
| TAU_ITR85 | 3622 | 2074 |
| TAU_ITR86 | 3610 | 2075 |

In some embodiments, the AAV particle comprises a viral genome which comprises a sequence which has a percent identity to any of SEQ ID NOs: 1990-2075, 2137-2168, 2171-2237, and 2260-2321. The viral genome may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to any of SEQ ID NOs: 1990-2075, 2137-2168, 2171-2237, and 2260-2321. The viral genome may have 1-10%, 10-20%, 30-40%, 50-60%, 50-70%, 50-80%, 50-90/o. 50-99%, 50-100/6, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to any of SEQ ID NOs: 1990-2075, 2137-2168, 2171-2237, and 2260-2321. As a non-limiting example, the viral genome comprises a sequence which has 80% identity to any of SEQ ID NO: 1990-2075, 2137-2168, 2171-2237, and 2260-2321. As another non-limiting example, the viral genome comprises a sequence which has 85% identity to any of SEQ ID NO: 1990-2075, 2137-2168, 2171-2237, and 2260-2321. As another non-limiting example, the viral genome comprises a sequence which has 90% identity to any of SEQ ID NO: 1990-2075, 2137-2168, 2171-2237, and 2260-2321. As another non-limiting example, the viral genome comprises a sequence which has 95% identity to any of SEQ ID NO: 1990-2075, 2137-2168, 2171-2237, and 2260-2321. As another non-limiting example, the viral genome comprises a sequence which has 99% identity to any of SEQ ID NO: 1990-2075, 2137-2168, 2171-2237, and 2260-2321.

In some embodiments, the AAV particles comprising anti-tau antibody polynucleotide sequences which comprise a nucleic acid sequence encoding at least one antibody heavy and/or light chain may be introduced into mammalian cells.

The AAV viral genomes encoding anti-tau antibody polynucleotides described herein may be useful in the fields of human disease, viruses, infections veterinary applications and a variety of in vivo and in vitro settings. In some embodiments, the AAV viral genomes encoding anti-tau antibody polynucleotides are used for the prevention and/or treatment of a tauopathy.

The viral genome of the AAV particles of the present disclosure may comprise any combination of the sequence regions described in Tables 7-14 encapsulated in any of the capsids listed in Table 1 or described herein.

In some embodiments, the AAV particle viral genome may comprise at least one sequence region as described in Tables 7-14. The regions may be located before or after any of the other sequence regions described herein. Viral genomes may further comprise more than one copy of one or more sequence regions as described in Tables 7-14.

In some embodiments, the AAV particle viral genome may comprise at least one inverted terminal repeat (ITR) region. The ITR region(s) may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR region for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As anon-limiting example, the viral genome comprises a 5′ ITR that is about 141 nucleotides in length. As a non-limiting example, the viral genome comprises a 5′ ITR that is about 130 nucleotides in length.

In some embodiments, the AAV particle viral genome comprises at least one 5′ inverted terminal repeat (5′ ITR) sequence region. Non-limiting examples of 5′ ITR sequence regions are described in Table 7.

TABLE 7
Inverted Terminal Repeat (ITR) Sequence Regions

| Sequence | Sequence | |
|---|---|---|
| Region Name | Length | SEQ ID NO |
| ITR1 | 130 | 2076 |
| ITR2 | 141 | 2077 |
| ITR3 | 130 | 2078 |
| ITR4 | 141 | 2079 |

In some embodiments, the AAV particle viral genome may have an ITR that comprises ITR1. In some embodiments, the AAV particle viral genome may have an ITR that comprises ITR2. In some embodiments, the AAV particle viral genome may have an ITR that comprises ITR3. In some embodiments, the AAV particle viral genome may have an ITR that comprises ITR4.

In some embodiments, the AAV particle viral genome may have two ITRs. As a non-limiting example, the two ITRs are ITR1 and ITR3. As a non-limiting example, the two ITRs are ITR1 and ITR4. As a non-limiting example, the two ITRs are ITR2 and ITR3. As a non-limiting example, the two ITRs are ITR2 and ITR4

In some embodiments, the AAV particle viral genome may comprise at least one promoter sequence region. The promoter sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 and more than 600 nucleotides. The length of the promoter region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, 590-600 and more than 600 nucleotides. As a non-limiting example, the viral genome comprises a promoter region that is about 260 nucleotides in length. As a non-limiting example, the viral genome comprises a promoter region that is about 283 nucleotides in length. As a non-limiting example, the viral genome comprises a promoter region that is about 299 nucleotides in length. As a non-limiting example, the viral genome comprises a promoter region that is about 365 nucleotides in length. As a non-limiting example, the viral genome comprises a promoter region that is about 380 nucleotides in length. As a non-limiting example, the viral genome comprises a promoter region that is about 382 nucleotides in length. As a non-limiting example, the viral genome comprises a promoter region that is about 557 nucleotides in length. As a non-limiting example, the viral genome comprises a promoter region that is about 654 nucleotides in length. As a non-limiting example, the viral genome comprises a promoter region that is about 699 nucleotides in length. As a non-limiting example, the viral genome comprises a promoter region that is about 1714 nucleotides in length. As a non-limiting example, the viral genome comprises a promoter region that is about 1715 nucleotides in length. As a non-limiting example, the viral genome comprises a promoter region that is about 1736 nucleotides in length.

In some embodiments, the AAV particle viral genome comprises at least one promoter sequence region. Non-limiting examples of promoter sequence regions are described in Table 8.

TABLE 8
Promoter Sequence Regions

| Sequence | Sequence | |
|---|---|---|
| Region Name | Length | SEQ ID NO |
| Promoter 1 | 1715 | 2080 |
| Promoter 2 | 299 | 2081 |
| Promoter 3 | 283 | 2082 |
| Promoter 4 | 260 | 2083 |
| Promoter 5 | 654 | 2084 |
| Promoter 6 | 699 | 2085 |
| Promoter 7 | 557 | 2086 |
| Promoter 8 | 382 | 2087 |
| Promoter 9 | 1736 | 2088 |
| Promoter 10 | 365 | 2089 |
| Promoter 11 | 1714 | 2238 |
| Promoter 12 | 380 | 2239 |

In some embodiments, the AAV particle viral genome comprises one promoter sequence region. In some embodiments, the promoter sequence region is Promoter 1. In some embodiments, the promoter sequence region is Promoter 2. In some embodiments, the promoter sequence region is Promoter 3. In some embodiments, the promoter sequence region is Promoter 4. In some embodiments, the promoter sequence region is Promoter 5. In some embodiments, the promoter sequence region is Promoter 6. In some embodiments, the promoter sequence region is Promoter 7. In some embodiments, the promoter sequence region is Promoter 8. In some embodiments, the promoter sequence region is Promoter 9. In some embodiments, the promoter sequence region is Promoter 10. In some embodiments, the promoter sequence region is Promoter 11. In some embodiments, the promoter sequence region is Promoter 12. In some embodiments, the promoter sequence region further comprises at least one promoter sub-region. As a non-limiting example, the promoter sequence is Promoter 1, further comprising Promoter 2 and Promoter 3 sub-regions.

In some embodiments, the AAV particle viral genome comprises more than one promoter sequence region. In some embodiments, the AAV particle viral genome comprises two promoter sequence regions. In some embodiments, the AAV particle viral genome comprises three promoter sequence regions.

In some embodiments, the AAV particle viral genome may comprise at least one exon sequence region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90,65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 54 nucleotides in length. As a non-limiting example, the viral genome comprises an exon region that is about 59 nucleotides in length. As a non-limiting example, the viral genome comprises an exon region that is about 102 nucleotides in length. As a non-limiting example, the viral genome comprises an exon region that is about 134 nucleotides in length.

In some embodiments, the AAV particle viral genome comprises at least one Exon sequence region. Non-limiting examples of Exon sequence regions are described in Table 9.

TABLE 9
Exon Sequence Regions

| Sequence | Sequence | |
|---|---|---|
| Region Name | Length | SEQ ID NO |
| Exon1 | 134 | 2090 |
| Exon2 | 102 | 2091 |
| Exon3 | 59 | 2092 |
| Exon4 | 53 | 2093 |
| Exon5 | 54 | 2094 |

In some embodiments, the AAV particle viral genome comprises one Exon sequence region. In some embodiments, the Exon sequence region is the Exon1 sequence region. In some embodiments, the Exon sequence region is the Exon2 sequence region. In some embodiments, the Exon sequence region is the Exon3 sequence region. In some embodiments, the Exon sequence region is the Exon4 sequence region. In some embodiments, the Exon sequence region is the Exon5 sequence region.

In some embodiments, the AAV particle viral genome comprises two Exon sequence regions. In some embodiments, the AAV particle viral genome comprises three Exon sequence regions. In some embodiments, the AAV particle viral genome comprises four Exon sequence regions. In some embodiments, the AAV particle viral genome comprises more than four Exon sequence regions.

In some embodiments, the AAV particle viral genome may comprise at least one intron sequence region. The intron region(s) may, independently, have a length such as, but not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350 and more than 350 nucleotides. The length of the intron region for the viral genome may be 25-35, 25-50, 35-45, 45-55, 50-75, 55-65, 65-75, 75-85, 75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises an intron region that is about 15 nucleotides in length. As a non-limiting example, the viral genome comprises an intron region that is about 32 nucleotides in length. As a non-limiting example, the viral genome comprises an intron region that is about 41 nucleotides in length. As a non-limiting example, the viral genome comprises an intron region that is about 53 nucleotides in length. As a non-limiting example, the viral genome comprises an intron region that is about 73 nucleotides in length. As a non-limiting example, the viral genome comprises an intron region that is about 168 nucleotides in length. As a non-limiting example, the viral genome comprises an intron region that is about 172 nucleotides in length. As a non-limiting example, the viral genome comprises an intron region that is about 292 nucleotides in length. As a non-limiting example, the viral genome comprises an intron region that is about 347 nucleotides in length. As a non-limiting example, the viral genome comprises an intron region that is about 387 nucleotides in length. As a non-limiting example, the viral genome comprises an intron region that is about 491 nucleotides in length. As a non-limiting example, the viral genome comprises an intron region that is about 566 nucleotides in length. As a non-limiting example, the viral genome comprises an intron region that is about 1074 nucleotides in length.

In some embodiments, the AAV particle viral genome comprises at least one intron sequence region. Non-limiting examples of intron sequence regions are described in Table 10.

TABLE 10
Intron Sequence Regions

| Sequence | Sequence | |
|---|---|---|
| Region Name | Length | SEQ ID NO |
| Intron1 | 32 | 2095 |
| Intron2 | 15 | 2096 |
| Intron3 | 347 | 2097 |
| Intron4 | 168 | 2098 |
| Intron5 | 73 | 2099 |
| Intron6 | 73 | 2100 |
| Intron7 | 73 | 2101 |
| Intron8 | 53 | 2102 |
| Intron9 | 172 | 2103 |
| Intron10 | 1074 | 2104 |
| Intron11 | 41 | 2105 |
| Intron12 | 566 | 2240 |
| Intron13 | 491 | 2256 |
| Intron14 | 387 | 2257 |
| Intron15 | 292 | 2258 |

In some embodiments, the AAV particle viral genome comprises one intron sequence region. In some embodiments, the intron sequence region is the Intron 1 sequence region. In some embodiments, the intron sequence region is the Intron2 sequence region. In some embodiments, the intron sequence region is the Intron3 sequence region. In some embodiments, the intron sequence region is the Intron4 sequence region. In some embodiments, the intron sequence region is the Intron5 sequence region. In some embodiments, the intron sequence region is the Intron6 sequence region. In some embodiments, the intron sequence region is the Intron7 sequence region. In some embodiments, the intron sequence region is the Intron8 sequence region. In some embodiments, the intron sequence region is the Intron9 sequence region. In some embodiments, the intron sequence region is the Intron10 sequence region. In some embodiments, the intron sequence region is the Intron11 sequence region. In some embodiments, the intron sequence region is the Intron12 sequence region. In some embodiments, the intron sequence region is the Intron13 sequence region. In some embodiments, the intron sequence region is the Intron14 sequence region. In some embodiments, the intron sequence region is the Intron15 sequence region.

In some embodiments, the AAV particle viral genome comprises two intron sequence regions. In some embodiments, the AAV particle viral genome comprises three intron sequence regions. In some embodiments, the AAV particle viral genome comprises more than three intron sequence regions.

In some embodiments, the AAV particle viral genome may comprise at least one signal sequence region, not derived from an antibody. In another embodiment, the signal sequence region may be derived from an antibody sequence. The signal sequence region(s) may, independently, have a length such as, but not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the signal region in the viral genome may be 10-15, 15-25, 25-35, 25-50, 35-45, 45-55, 50-75, 55-65, 65-75, 75-85, 75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises a signal sequence region that is about 12 nucleotides in length. As a non-limiting example, the viral genome comprises a signal sequence region that is about 57 nucleotides in length. As a non-limiting example, the viral genome comprises a signal sequence region that is about 66 nucleotides in length. As a non-limiting example, the viral genome comprises a signal sequence region that is about 69 nucleotides in length. As a non-limiting example, the viral genome comprises a signal sequence region that is about 72 nucleotides in length. As a non-limiting example, the viral genome comprises a signal sequence region that is about 78 nucleotides in length. As a non-limiting example, the viral genome comprises a signal sequence region that is about 81 nucleotides in length. As a non-limiting example, the viral genome comprises a signal sequence region that is about 84 nucleotides in length. As a non-limiting example, the viral genome comprises a signal sequence region that is about 93 nucleotides in length. As a non-limiting example, the viral genome comprises a signal sequence region that is about 96 nucleotides in length. As a non-limiting example, the viral genome comprises a signal sequence region that is about 411 nucleotides in length.

In some embodiments, the AAV particle viral genome comprises at least one signal sequence region. Non-limiting examples of signal sequence regions not derived from an antibody sequence are described in Table 11.

TABLE 11
Signal Sequence Regions

| Sequence | Sequence | |
|---|---|---|
| Region Name | Length | SEQ ID NO |
| Signal1 | 84 | 2106 |
| Signal2 | 93 | 2107 |
| Signal3 | 96 | 2108 |
| Signal4 | 66 | 2109 |
| Signal5 | 72 | 2110 |
| Signal6 | 93 | 2111 |
| Signal7 | 69 | 2112 |
| Signal8 | 81 | 2113 |
| Signal9 | 12 | 2114 |
| Signal10 | 81 | 2115 |
| Signal11 | 66 | 2116 |
| Signal12 | 78 | 2117 |
| Signal13 | 57 | 1740 |
| Signal14 | 57 | 1741 |
| Signal15 | 57 | 1861 |
| Signal16 | 411 | 2241 |

In some embodiments, the AAV particle viral genome comprises one signal sequence region. In some embodiments, the signal sequence region is the Signal1 sequence region. In some embodiments, the signal sequence region is the Signal2 sequence region. In some embodiments, the signal sequence region is the Signal3 sequence region. In some embodiments, the signal sequence region is the Signal4 sequence region. In some embodiments, the signal sequence region is the Signal5 sequence region. In some embodiments, the signal sequence region is the Signal6 sequence region. In some embodiments, the signal sequence region is the Signal7 sequence region. In some embodiments, the signal sequence region is the Signal8 sequence region. In some embodiments, the signal sequence region is the Signal9 sequence region. In some embodiments, the signal sequence region is the Signal10 sequence region. In some embodiments, the signal sequence region is the Signal11 sequence region. In some embodiments, the signal sequence region is the Signal12 sequence region. In some embodiments, the signal sequence region is the Signal13 sequence region. In some embodiments, the signal sequence region is the Signal14 sequence region. In some embodiments, the signal sequence region is the Signal15 sequence region. In some embodiments, the signal sequence region is the Signal16 sequence region.

In some embodiments, the signal sequence is derived from an antibody sequence. As a non-limiting example, a signal sequence may be derived from the heavy chain or the light chain of an anti-tau antibody, such as, but not limited to, IPN002, PHF1 and/or MC1. While not wishing to be bound by theory, the first approximately 57 nucleotides of an antibody heavy chain or light chain sequence may be considered a signal sequence. Non-limiting examples of antibody derived signal sequences include Abi (SEQ ID NO: 1740), Ab2 (SEQ ID NO: 1741), and Ab122-124 (SEQ ID NO: 1861-1863), herein.

In some embodiments, the AAV particle viral genome comprises one signal sequence region. In some embodiments, the AAV particle viral genome comprises two signal sequence regions. In some embodiments, the AAV particle viral genome comprises three signal sequence regions. In some embodiments, the AAV particle viral genome comprises more than three signal sequence regions. In some embodiments, the signal sequences of a viral genome comprising more than one signal sequence, are the same. In another embodiment, the signal sequences of a viral genome comprising more than one signal sequence, are not the same.

In some embodiments, the AAV particle viral genome may comprise at least one tag sequence region. As used herein, the term “tag” indicates a polynucleotide sequence appended to the payload, that once expressed may be used to identify the expressed payload. Alternatively, the term “tag” may indicate a polynucleotide sequence appended to the payload that signals for retention of the expressed payload in a particular region of the cell (e.g., endoplasmic reticulum). The tag sequence region(s) may, independently, have a length such as, but not limited to, 10, 11, 12, 13, 14, 15, 16, 17, 18, 19, 20, 21, 22, 23, 24, 25, 26, 27, 28, 29, 30 or more than 30 nucleotides. The length of the tag sequence region in the viral genome may be 10-15, 15-20, 20-25, 25-30, or more than 30 nucleotides. As a non-limiting example, the viral genome comprises a tag sequence region that is about 18 nucleotides in length. As a non-limiting example, the viral genome comprises a tag sequence region that is about 21 nucleotides in length. As a non-limiting example, the viral genome comprises a tag sequence region that is about 27 nucleotides in length.

In some embodiments, the AAV particle viral genome comprises at least one tag sequence region. Non-limiting examples of tag sequence regions are described in Table 12.

TABLE 12
Tag Sequence Regions

| Sequence | Sequence | |
|---|---|---|
| Region Name | Length | SEQ ID NO |
| Tag1 | 27 | 2118 |
| Tag2 | 21 | 2119 |
| Tag3 | 18 | 2120 |
| Tag4 | 18 | 2121 |
| Tag5 | 18 | 2255 |

In some embodiments, the AAV particle viral genome comprises one tag sequence region. In some embodiments, the tag sequence region is the Tag1 sequence region. In some embodiments, the tag sequence region is the Tag2 sequence region. In some embodiments, the tag sequence region is the Tag3 sequence region. In some embodiments, the tag sequence region is the Tag4 sequence region. In some embodiments, the tag sequence region is the Tag5 sequence region.

In some embodiments, the AAV particle viral genome comprises more than one tag sequence region. In some embodiments, the AAV particle viral genome comprises two tag sequence regions. In some embodiments, the AAV particle viral genome comprises three tag sequence regions. In some embodiments, the AAV particle viral genome comprises more than three tag sequence regions.

In some embodiments, the AAV particle viral genome may comprise at least one polyadenylation sequence region. The polyadenylation sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the polyadenylation sequence region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a polyadenylation sequence region that is about 127 nucleotides in length. As a non-limiting example, the viral genome comprises a polyadenylation sequence region that is about 477 nucleotides in length. As a non-limiting example, the viral genome comprises a polyadenylation sequence region that is about 552 nucleotides in length.

In some embodiments, the AAV particle viral genome comprises at least one polyadenylation (polyA) sequence region. Non-limiting examples of polyA sequence regions are described in Table 13.

TABLE 13
Poly-A Signal Sequence Regions

| Sequence | Sequence | |
|---|---|---|
| Region Name | Length | SEQ ID NO |
| PolyA1 | 127 | 2122 |
| PolyA2 | 477 | 2123 |
| PolyA3 | 552 | 2124 |

In some embodiments, the AAV particle viral genome comprises one polyA sequence region. In some embodiments, the polyA sequence region is the PolyA1 sequence. In some embodiments, the polyA sequence region is the PolyA2 sequence. In some embodiments, the polyA signal sequence region is the PolyA3 sequence.

In some embodiments, the AAV particle viral genome comprises more than one polyA sequence region.

In some embodiments, the AAV particle viral genome may comprise at least one or multiple filler sequence regions. The filler region(s) may, independently, have a length such as, but not limited to, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, and 3250 nucleotides. The length of any filler region for the viral genome may be 50-100, 100-150, 150-200, 200-250, 250-300, 300-350, 350-400, 400-450, 450-500, 500-550, 550-600, 600-650, 650-700, 700-750, 750-800, 800-850, 850-900,900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides. As a non-limiting example, the viral genome comprises a filler region that is about 1153 nucleotides in length. As a non-limiting example, the viral genome comprises a filler region that is about 1240 nucleotides in length.

In some embodiments, the AAV particle viral genome comprises at least one filler sequence region. Non-limiting examples of filler sequence regions are described in Table 14.

TABLE 14
Filler Sequence Regions

| Sequence | Sequence | |
|---|---|---|
| Region Name | Length | SEQ ID NO |
| FILLER1 | 1153 | 2125 |
| FILLER2 | 1240 | 2126 |

In some embodiments, the AAV particle viral genome comprises filler sequence region FILLER1. In some embodiments, the AAV particle viral genome comprises filler sequence region FILLER2. In some embodiments, the AAV particle viral genome comprises both FILLER1 and FILLER2. In some embodiments, the AAV particle viral genome does not comprise a filler sequence region.

In some embodiments, the AAV particle viral genome may comprise any of the sequences shown in Tables 15-91.

TABLE 15
Sequence Regions in ITR to ITR Sequences

| | |
|---|---|
| TAU_ITR1 | TAU_ITR2 |
| (SEQ ID NO: 1990) | (SEQ ID NO: 1991) |

| Sequence | Region | Region | Region | Region |
|---|---|---|---|---|
| Regions | SEQ ID NO | length | SEQ ID NO | length |
| 5′ ITR | 2076 | 130 | 2076 | 130 |
| CAG | 2080 | 1715 | 2080 | 1715 |
| promoter | | | | |
| CMVie | 2081 | 299 | 2081 | 299 |
| CBA min. | 2082 | 283 | 2082 | 283 |
| Signal | 1740 | 57 | 1740 | 57 |
| VH | 1821 | 351 | 1821 | 351 |
| Constant | 1742 | 969 | 1742 | 969 |
| Linker | 1724 | 12 | 1725 | 12 |
| Linker | 1726 | 54 | 1727 | 75 |
| Signal | 1861 | 57 | 1861 | 57 |
| VL | 1939 | 336 | 1939 | 336 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 1990 (TAU_ITR1) which comprises a 5′ inverted terminal repeat (ITR) sequence region and a 3′ ITR sequence region, a CAG promoter comprising a CMVie region and a minimal CBA promoter region, an IPN002 antibody heavy chain signal sequence, an IPN002 antibody heavy chain variable region and heavy chain constant region, a furin cleavage site, a T2A linker, an IPN002 antibody light chain signal sequence, an IPN002 antibody light chain variable region and light chain constant region, and a rabbit globin polyadenylation sequence.

In some embodiments, the AAV particle genome comprises SEQ ID NO: 3815 (TAU_ITR2) which comprises a 5 inverted terminal repeat (ITR) sequence region and a 3′ ITR sequence region, a CAG promoter comprising a CMVie region and a minimal CBA promoter region, an IPN002 antibody heavy chain signal sequence, an IPN002 antibody heavy chain variable region and heavy chain constant region, a furin cleavage site, an F2A linker, an IPN002 antibody light chain signal sequence, an IPN002 antibody light chain variable region and light chain constant region, and a rabbit globin polyadenylation sequence.

TABLE 16
Sequence Regions in ITR to ITR Sequences

| | |
|---|---|
| TAU_ITR3 | TAU_ITR4 |
| (SEQ ID NO: 1992) | (SEQ ID NO: 1993) |

| Sequence | Region | Region | Region | Region |
|---|---|---|---|---|
| Regions | SEQ ID NO | length | SEQ ID NO | length |
| 5′ ITR | 2076 | 130 | 2076 | 130 |
| CB | 2083 | 260 | 2083 | 260 |
| promoter | | | | |
| Exon | 2090 | 134 | 2090 | 134 |
| Intron | 2095 | 32 | 2095 | 32 |
| Intron | 2097 | 347 | 2097 | 347 |
| Exon | 2093 | 53 | 2093 | 53 |
| Signal | 1740 | 57 | 1740 | 57 |
| VH | 1821 | 351 | 1821 | 351 |
| Constant | 1742 | 969 | 1742 | 969 |
| Linker | 1725 | 12 | 1724 | 12 |
| Linker | 1727 | 75 | 1726 | 54 |
| Signal | 1861 | 57 | 1861 | 57 |
| VL | 1939 | 336 | 1939 | 336 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 1992 (TAU_ITR3) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody heavy chain signal sequence, an IPN002 antibody heavy chain variable region and heavy chain constant region, a furin cleavage site, an F2A linker, an IPN002 antibody light chain signal sequence, an IPN002 antibody light chain variable region and light chain constant region, and a rabbit globin polyadenylation sequence.

In some embodiments, the AAV particle genome comprises SEQ ID NO: 1993 (TAU_ITR4) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody heavy chain signal sequence, an IPN002 antibody heavy chain variable region and heavy chain constant region, a furin cleavage site, a T2A linker, an IPN002 antibody light chain signal sequence, an IPN002 antibody light chain variable region and light chain constant region, and a rabbit globin polyadenylation sequence.

TABLE 17
Sequence Regions in ITR to ITR Sequences

| | TAU_ITR5 | | TAU_ITR6 | | TAU_ITR7 | | TAU_ITR8 | |
|---|---|---|---|---|---|---|---|---|
| | (SEQ ID NO: | | (SEQ ID NO: | | (SEQ ID NO: | | (SEQ ID NO: | |
| | 1994) | | 1995) | | 1996) | | 1997) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2085 | 699 | 2085 | 699 | 2086 | 557 | 2086 | 557 |
| Exon | 2090 | 134 | 2090 | 134 | 2090 | 134 | 2090 | 134 |
| Intron | 2095 | 32 | 2095 | 32 | 2095 | 32 | 2095 | 32 |
| Intron | 2097 | 347 | 2097 | 347 | 2097 | 347 | 2097 | 347 |
| Exon | 2093 | 53 | 2093 | 53 | 2093 | 53 | 2093 | 53 |
| Signal | 1740 | 57 | 1740 | 57 | 1740 | 57 | 1740 | 57 |
| VH | 1821 | 351 | 1821 | 351 | 1821 | 351 | 1821 | 351 |
| Constant | 1742 | 969 | 1742 | 969 | 1742 | 969 | 1742 | 969 |
| Linker | 1725 | 12 | 1724 | 12 | 1725 | 12 | 1724 | 12 |
| Linker | 1727 | 75 | 1726 | 54 | 1727 | 75 | 1726 | 54 |
| Signal | 1861 | 57 | 1861 | 57 | 1861 | 57 | 1861 | 57 |
| VL | 1939 | 336 | 1939 | 336 | 1939 | 336 | 1939 | 336 |
| Constant | 1864 | 321 | 1864 | 321 | 1864 | 321 | 1864 | 321 |
| Poly A | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 1994 (TAU_ITR5) which comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, an IPN002 antibody heavy chain signal sequence, an IPN002 antibody heavy chain variable region and heavy chain constant region, a furin cleavage site, an F2A linker, an IPN002 antibody light chain signal sequence, an IPN002 antibody light chain variable region and light chain constant region, and a rabbit globin polyadenylation sequence.

In some embodiments, the AAV particle genome comprises SEQ ID NO: 1995 (TAU_ITR6) which comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, an IPN002 antibody heavy chain signal sequence, an IPN002 antibody heavy chain variable region and heavy chain constant region, a furin cleavage site, a T2A linker, an IPN002 antibody light chain signal sequence, an IPN002 antibody light chain variable region and light chain constant region, and a rabbit globin polyadenylation sequence.

In some embodiments, the AAV particle genome comprises SEQ ID NO: 1996 (TAU_ITR7) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, an IPN002 antibody heavy chain signal sequence, an IPN002 antibody heavy chain variable region and heavy chain constant region, a furin cleavage site, an F2A linker, an IPN002 antibody light chain signal sequence, an IPN002 antibody light chain variable region and light chain constant region, and a rabbit globin polyadenylation sequence.

In some embodiments, the AAV particle genome comprises SEQ ID NO: 1997 (TAU_ITR8) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, an IPN002 antibody heavy chain signal sequence, an IPN002 antibody heavy chain variable region and heavy chain constant region, a furin cleavage site, a T2A linker, an IPN002 antibody light chain signal sequence, an IPN002 antibody light chain variable region and light chain constant region, and a rabbit globin polyadenylation sequence.

TABLE 18
Sequence Regions in ITR to ITR Sequences

| | TAU_ITR9 | | TAU_ITR10 | |
|---|---|---|---|---|
| | (SEQ ID NO: 1998) | | (SEQ ID NO: 1999) | |
| Sequence | Region | Region | Region | Region |
| Regions | SEQ ID NO | length | SEQ ID NO | length |
| 5′ ITR | 2076 | 130 | 2076 | 130 |
| CAG promoter | 2080 | 1715 | 2080 | 1715 |
| CMVie | 2081 | 299 | 2081 | 299 |
| CBA min. | 2082 | 283 | 2082 | 283 |
| Signal | 1861 | 57 | 1861 | 57 |
| VL | 1939 | 336 | 1939 | 336 |
| Constant | 1864 | 321 | 1864 | 321 |
| Linker | 1727 | 75 | 1726 | 54 |
| Signal | 1740 | 57 | 1740 | 57 |
| VH | 1821 | 351 | 1821 | 351 |
| Constant | 1742 | 969 | 1742 | 969 |
| PolyA | 2122 | 127 | 2122 | 127 |
| 3′ ITR | 2078 | 130 | 2078 | 130 |

In some embodiments, the AAV particle genome comprises SEQ ID NO: 1998 (TAU_ITR9) which comprises a 5′ inverted terminal repeat (ITR) sequence region and a 3′ ITR sequence region, a CAG promoter comprising a CMVie region and a minimal CBA promoter region, an IPN002 antibody light chain signal sequence, an IPN002 antibody light chain variable region and light chain constant region, an F2A linker, an IPN002 antibody heavy chain signal sequence, an IPN002 antibody heavy chain variable region and heavy chain constant region, and a rabbit globin polyadenylation sequence.

In some embodiments, the AAV particle genome comprises SEQ ID NO: 1999 (TAU_ITR10) which comprises a 5′ inverted terminal repeat (ITR) sequence region and a 3′ ITR sequence region, a CAG promoter comprising a CMVie region and a minimal CBA promoter region, an IPN002 antibody light chain signal sequence, an IPN002 antibody light chain variable region and light chain constant region, a T2A linker, an IPN002 antibody heavy chain signal sequence, an IPN002 antibody heavy chain variable region and heavy chain constant region, and a rabbit globin polyadenylation sequence.

TABLE 19
Sequence Regions in ITR to ITR Sequences

| | |
|---|---|
| TAU_ITR11 | TAU_ITR87 |
| (SEQ ID NO: 2000) | (SEQ ID NO: 2137) |

| Sequence | Region | Region | Region | Region |
|---|---|---|---|---|
| Regions | SEQ ID NO | length | SEQ ID NO | length |
| 5′ ITR | 2076 | 130 | 2076 | 130 |
| Promoter | 2083 | 260 | 2083 | 260 |
| Exon | 2090 | 134 | 2090 | 134 |
| Intron | 2095 | 32 | 2095 | 32 |
| Intron | 2097 | 347 | 2097 | 347 |
| Exon | 2093 | 53 | 2093 | 53 |
| Signal | 1861 | 57 | 1861 | 57 |
| VL | 1939 | 336 | 1939 | 336 |
| Constant | 1864 | 321 | 1864 | 321 |
| Linker | 1727 | 75 | 1726 | 54 |
| Signal | 1740 | 57 | 1740 | 57 |
| VH | 1821 | 351 | 1821 | 351 |
| Constant | 1742 | 969 | 1742 | 969 |
| PolyA | 2122 | 127 | 2122 | 127 |
| 3′ ITR | 2078 | 130 | 2078 | 130 |

In some embodiments, the AAV particle genome comprises SEQ ID NO: 2000 (TAU_ITR11) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, an IPN002 antibody light chain variable region and light chain constant region, an F2A linker, an IPN002 antibody heavy chain signal sequence, an IPN002 antibody heavy chain variable region and heavy chain constant region, and a rabbit globin polyadenylation sequence.

In some embodiments, the AAV particle genome comprises SEQ ID NO: 2137 (TAU_ITR87) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, an IPN002 antibody light chain variable region and light chain constant region, an T2A linker, an IPN002 antibody heavy chain signal sequence, an IPN002 antibody heavy chain variable region and heavy chain constant region, and a rabbit globin polyadenylation sequence.

TABLE 20
Sequence Regions in ITR to ITR Sequences

| | TAU_ITR12 | | TAU_ITR13 | | TAU_ITR14 | | TAU_ITR15 | |
|---|---|---|---|---|---|---|---|---|
| | (SEQ ID NO: | | (SEQ ID NO: | | (SEQ ID NO: | | (SEQ ID NO: | |
| | 2001) | | 2002) | | 2003) | | 2004) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2085 | 699 | 2085 | 699 | 2086 | 557 | 2086 | 557 |
| Exon | 2090 | 134 | 2090 | 134 | 2090 | 134 | 2090 | 134 |
| Intron | 2095 | 32 | 2095 | 32 | 2095 | 32 | 2095 | 32 |
| Intron | 2097 | 347 | 2097 | 347 | 2097 | 347 | 2097 | 347 |
| Exon | 2093 | 53 | 2093 | 53 | 2093 | 53 | 2093 | 53 |
| Signal | 1861 | 57 | 1861 | 57 | 1861 | 57 | 1861 | 57 |
| VL | 1939 | 336 | 1939 | 336 | 1939 | 336 | 1939 | 336 |
| Constant | 1864 | 321 | 1864 | 321 | 1864 | 321 | 1864 | 321 |
| Linker | 1727 | 75 | 1726 | 54 | 1727 | 75 | 1726 | 54 |
| Signal | 1740 | 57 | 1740 | 57 | 1740 | 57 | 1740 | 57 |
| VH | 1821 | 351 | 1821 | 351 | 1821 | 351 | 1821 | 351 |
| Constant | 1742 | 969 | 1742 | 969 | 1742 | 969 | 1742 | 969 |
| Poly A | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2001 (TAU_ITR12) which comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, an IPN002 antibody light chain signal sequence, an IPN002 antibody light chain variable region and light chain constant region, an F2A linker, an IPN002 antibody heavy chain signal sequence, an IPN002 antibody heavy chain variable region and heavy chain constant region, and a rabbit globin polyadenylation sequence.

In some embodiments, the AAV particle genome comprises SEQ ID NO: 2002 (TAU_ITR13) which comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, an IPN002 antibody light chain signal sequence, an IPN002 antibody light chain variable region and light chain constant region, a T2A linker, an IPN002 antibody heavy chain signal sequence, an IPN002 antibody heavy chain variable region and heavy chain constant region, and a rabbit globin polyadenylation sequence.

In some embodiments, the AAV particle genome comprises SEQ ID NO: 2003 (TAU_ITR14) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, an IPN002 antibody light chain signal sequence, an IPN002 antibody light chain variable region and light chain constant region, an F2A linker, an IPN002 antibody heavy chain signal sequence, an IPN002 antibody heavy chain variable region and heavy chain constant region, and a rabbit globin polyadenylation sequence.

In some embodiments, the AAV particle genome comprises SEQ ID NO: 2004 (TAU_ITR15) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, an IPN002 antibody light chain signal sequence, an IPN002 antibody light chain variable region and light chain constant region, a T2A linker, an IPN002 antibody heavy chain signal sequence, an IPN002 antibody heavy chain variable region and heavy chain constant region, and a rabbit globin polyadenylation sequence.

TABLE 21
Sequence Regions in ITR to ITR Sequences

| | TAU_ITR16 | | TAU_ITR17 | | TAU_ITR18 | | TAU_ITR19 | | TAU_ITR20 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2005) | | 2006) | | 2007) | | 2008) | | 2009) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2083 | 260 | 2083 | 260 | 2083 | 260 | 2083 | 260 | 2083 | 260 |
| Exon | 2090 | 134 | 2090 | 134 | 2090 | 134 | 2090 | 134 | 2090 | 134 |
| Intron | 2095 | 32 | 2095 | 32 | 2095 | 32 | 2095 | 32 | 2095 | 32 |
| Intron | 2097 | 347 | 2097 | 347 | 2097 | 347 | 2097 | 347 | 2097 | 347 |
| Exon | 2093 | 53 | 2093 | 53 | 2093 | 53 | 2093 | 53 | 2093 | 53 |
| Signal | 1861 | 57 | 1861 | 57 | 1861 | 57 | 1861 | 57 | 1861 | 57 |
| VL | 1921 | 321 | 1922 | 321 | 1923 | 321 | 1924 | 321 | 1925 | 321 |
| Constant | 1864 | 321 | 1864 | 321 | 1864 | 321 | 1864 | 321 | 1864 | 321 |
| Linker | 1726 | 54 | 1726 | 54 | 1726 | 54 | 1726 | 54 | 1726 | 54 |
| Signal | 1740 | 57 | 1740 | 57 | 1740 | 57 | 1740 | 57 | 1740 | 57 |
| VH | 1804 | 345 | 1805 | 345 | 1806 | 345 | 1807 | 345 | 1808 | 345 |
| Constant | 1743 | 972 | 1743 | 972 | 1743 | 972 | 1743 | 972 | 1743 | 972 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2005 (TAU_ITR16) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (Bioinformatics) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2006 (TAU_ITR17) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (EMBOSS) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2007 (TAU_ITR18) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (GeneInfinity) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2008 (TAU_ITR19) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (GregThatcher) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2009 (TAU_ITR20) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (IDT) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

TABLE 22
Sequence Regions in ITR to ITR Sequences

| | TAU_ITR21 | | TAU_ITR22 | | TAU_ITR23 | | TAU_ITR24 | | TAU_ITR25 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2010) | | 2011) | | 2012) | | 2013) | | 2014) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2083 | 260 | 2083 | 260 | 2083 | 260 | 2083 | 260 | 2083 | 260 |
| Exon | 2090 | 134 | 2090 | 134 | 2090 | 134 | 2090 | 134 | 2090 | 134 |
| Intron | 2095 | 32 | 2095 | 32 | 2095 | 32 | 2095 | 32 | 2095 | 32 |
| Intron | 2097 | 347 | 2097 | 347 | 2097 | 347 | 2097 | 347 | 2097 | 347 |
| Exon | 2093 | 53 | 2093 | 53 | 2093 | 53 | 2093 | 53 | 2093 | 53 |
| Signal | 1861 | 57 | 1861 | 57 | 1861 | 57 | 1861 | 57 | 1861 | 57 |
| VL | 1926 | 321 | 1927 | 321 | 1928 | 321 | 1929 | 321 | 1930 | 321 |
| Constant | 1864 | 321 | 1864 | 321 | 1864 | 321 | 1864 | 321 | 1864 | 321 |
| Linker | 1726 | 54 | 1726 | 54 | 1726 | 54 | 1726 | 54 | 1726 | 54 |
| Signal | 1740 | 57 | 1740 | 57 | 1740 | 57 | 1740 | 57 | 1740 | 57 |
| VH | 1809 | 345 | 1810 | 345 | 1811 | 345 | 1812 | 345 | 1813 | 345 |
| Constant | 1743 | 972 | 1743 | 972 | 1743 | 972 | 1743 | 972 | 1743 | 972 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2010 (TAU_ITR21) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (InSilico) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2011 (TAU_ITR22) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (MolBio) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2012 (TAU_ITR23) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (N2P) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2013 (TAU_ITR24) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (SnapGene) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

In some embodiments, the AAV particle genome comprises SEQ ID NO: 2014 (TAU_ITR25) which comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an IPN002 antibody light chain signal sequence, a codon-optimized (Vector NTI) C10.2 antibody light chain variable region, a light chain constant region, a T2A linker, an IPN002 antibody heavy chain signal sequence, a codon-optimized C10.2 antibody heavy chain variable region, a heavy chain constant region, and a rabbit globin polyadenylation sequence.

TABLE 23
Sequence Regions in ITR to ITR Sequences

| | TAU_ITR26 | | TAU_ITR27 | | TAU_ITR28 | | TAU_ITR29 | | TAU_ITR30 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2015) | | 2016) | | 2017) | | 2018) | | 2019) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2077 | 141 | 2077 | 141 | 2077 | 141 | 2077 | 141 | 2077 | 141 |
| Promoter | 2084 | 654 | 2084 | 654 | 2084 | 654 | 2084 | 654 | 2084 | 654 |
| Exon | 2090 | 134 | 2090 | 134 | 2090 | 134 | 2090 | 134 | 2090 | 134 |
| Intron | 2095 | 32 | 2095 | 32 | 2095 | 32 | 2095 | 32 | 2095 | 32 |
| Intron | 2097 | 347 | 2097 | 347 | 2097 | 347 | 2097 | 347 | 2097 | 347 |
| Exon | 2093 | 53 | 2093 | 53 | 2093 | 53 | 2093 | 53 | 2093 | 53 |
| Signal | 1740 | 57 | — | — | 1740 | 57 | 1740 | 57 | 1740 | 57 |
| VH | 1854 | 354 | 1859 | 354 | 1852 | 354 | 1850 | 354 | 1856 | 354 |
| Constant | 1742 | 969 | 1742 | 969 | 1742 | 969 | 1742 | 969 | 1742 | 969 |
| Linker | 1724 | 12 | 1724 | 12 | 1724 | 12 | 1724 | 12 | 1724 | 12 |
| Linker | 1726 | 54 | 1726 | 54 | 1726 | 54 | 1726 | 54 | 1726 | 54 |
| Signal | 1861 | 57 | 1861 | 57 | 1861 | 57 | 1861 | 57 | 1861 | 57 |
| VL | 1975 | 321 | 1980 | 321 | 1973 | 321 | 1971 | 321 | 1977 | 321 |
| Constant | 1864 | 321 | 1864 | 321 | 1864 | 321 | 1864 | 321 | 1864 | 321 |
| Poly A | 2123 | 477 | 2123 | 477 | 2123 | 477 | 2123 | 477 | 2123 | 477 |
| 3′ HR | 2079 | 141 | 2079 | 141 | 2079 | 141 | 2079 | 141 | 2079 | 141 |

In some embodiments, the AAV particle genome comprises SEQ ID NO: 2015 (TAU_ITR26) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (GeneScript) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2016 (TAU_ITR27) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a codon-optimized (SnapGene) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2017 (TAU_ITR28) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (EMBOSS) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2018 (TAU_ITR29) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (Bioinformatics) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2019 (TAU_ITR30) which comprises a 5′ inverted terminal repeat (ITR) sequence region and a 3′ ITR sequence region, a CBA promotcr, an el exon 1 region, an ie1 intron 1 region, a human beta-globin intron region, a human beta-globin exon region, an IPN002 antibody heavy chain signal sequence, a codon-optimized (NUS) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

TABLE 24
Sequence Regions in ITR to ITR Sequences

| | TAU_ITR31 | | TAU_ITR32 | | TAU_ITR33 | | TAU_ITR34 | | TAU_ITR35 | |
|---|---|---|---|---|---|---|---|---|---|---|
| | (SEQ ID NO: | | (SEQ ID NO: | | (SEQ ID NO: | | (SEQ ID NO | | (SEQ ID NO: | |
| | 2020) | | 2021) | | 2022) | | 2023) | | 2024) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2077 | 141 | 2077 | 141 | 2077 | 141 | 2077 | 141 | 2077 | 141 |
| Promoter | 2084 | 654 | 2084 | 654 | 2084 | 654 | 2084 | 654 | 2084 | 654 |
| Exon | 2090 | 134 | 2090 | 134 | 2090 | 134 | 2090 | 134 | 2090 | 134 |
| Intron | 2095 | 32 | 2095 | 32 | 2095 | 32 | 2095 | 32 | 2095 | 32 |
| Intron | 2097 | 347 | 2097 | 347 | 2097 | 347 | 2097 | 347 | 2097 | 347 |
| Exon | 2093 | 53 | 2093 | 53 | 2093 | 53 | 2093 | 53 | 2093 | 53 |
| Signal | 1740 | 57 | 1740 | 57 | 1740 | 57 | 1740 | 57 | 1740 | 57 |
| VH | 1857 | 354 | 1853 | 354 | 1855 | 354 | 1851 | 354 | 1858 | 354 |
| Constant | 1742 | 969 | 1742 | 969 | 1742 | 969 | 1742 | 969 | 1742 | 969 |
| Linker | 1724 | 12 | 1724 | 12 | 1724 | 12 | 1724 | 12 | 1724 | 12 |
| Linker | 1726 | 54 | 1726 | 54 | 1726 | 54 | 1726 | 54 | 1726 | 54 |
| Signal | 1861 | 57 | 1861 | 57 | 1861 | 57 | 1861 | 57 | 1861 | 57 |
| VL | 1978 | 321 | 1974 | 321 | 1976 | 321 | 1972 | 321 | 1979 | 321 |
| Constant | 1864 | 321 | 1864 | 321 | 1864 | 321 | 1864 | 321 | 1864 | 321 |
| Poly A | 2123 | 477 | 2123 | 477 | 2123 | 477 | 2123 | 477 | 2123 | 477 |
| 3′ ITR | 2079 | 141 | 2079 | 141 | 2079 | 141 | 2079 | 141 | 2079 | 141 |

In some embodiments, the AAV particle genome comprises SEQ ID NO: 2020 (TAU_ITR31) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (NUS2) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2021 (TAU_ITR32) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (GeneInfinity) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2022 (TAU_ITR33) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (IDT) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2023 (TAU_ITR34) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (Bioinformatics 2) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

In some embodiments, the AAV particle genome comprises SEQ ID NO: 2024 (TAU_ITR35) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an IPN002 antibody heavy chain signal sequence, a codon-optimized (NUS3) PT3 antibody heavy chain variable region, a heavy chain constant region, a furin cleavage site, a T2A linker, an IPN002 antibody light chain signal sequence, a codon-optimized PT3 antibody light chain variable region, a light chain constant region, and a human growth hormone polyadenylation sequence.

TABLE 25
Sequence Regions in ITR to ITR Sequences

| | TAU_ITR36 | | TAU_ITR37 | | TAU_ITR38 | | TAU_ITR39 | | TAU_ITR40 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2025) | | 2026) | | 2027) | | 2028) | | 2029) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2077 | 141 | 2077 | 141 | 2077 | 141 | 2077 | 141 | 2077 | 141 |
| CAG | 2080 | 1715 | 2080 | 1715 | 2080 | 1715 | 2080 | 1715 | 2080 | 1715 |
| promoter | | | | | | | | | | |
| CMVie | 2081 | 299 | 2081 | 299 | 2081 | 299 | 2081 | 299 | 2081 | 299 |
| CBA min. | 2082 | 283 | 2082 | 283 | 2082 | 283 | 2082 | 283 | 2082 | 283 |
| Signal | 1741 | 57 | 2106 | 84 | 2106 | 84 | 1741 | 57 | 1741 | 57 |
| VH | 1839 | 351 | 1839 | 351 | 1839 | 351 | 1839 | 351 | 1839 | 351 |
| Linker | 1730 | 45 | 1730 | 45 | 1730 | 45 | 1730 | 45 | 1730 | 45 |
| VL | 1957 | 339 | 1957 | 339 | 1957 | 339 | 1957 | 339 | 1957 | 339 |
| Tag | 2118 | 27 | 2119 | 21 | 2121 | 18 | 2118 | 27 | 2121 | 18 |
| Poly A | 2122 | 127 | 2123 | 477 | 2123 | 477 | 2123 | 477 | 2123 | 477 |
| Filler | 2125 | 1153 | 2126 | 1240 | 2126 | 1240 | 2126 | 1240 | 2126 | 1240 |
| 3′ ITR | 2079 | 141 | 2079 | 141 | 2079 | 141 | 2079 | 141 | 2079 | 141 |

In some embodiments, the AAV particle genome comprises SEQ ID NO: 2025 (TAU_ITR36) which comprises a 5′ inverted terminal repeat (ITR) sequence region and a 3′ ITR sequence region, a CAG promoter comprising a CMVie region and a minimal CBA promoter region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, a rabbit globin polyadenylation signal sequence and a human albumin derived filler sequence.

In some embodiments, the AAV particle genome comprises SEQ ID NO: 2026 (TAU_ITR37) which comprises a 5′ inverted terminal repeat (ITR) sequence region and a 3′ ITR sequence region, a CAG promoter comprising a CMVie region and a minimal CBA promoter region, a human growth hormone-2 signal sequence, a PHF1 antibody heavy chain variable region, a (G4S)3 linker (“(G4S)3” disclosed as SEQ ID NO: 4537), a PHF1 antibody light chain variable region, an SEKDEL tag (“SEKDEL” disclosed as SEQ ID NO: 4546), a human growth hormone polyadenylation signal sequence and a human albumin derived filler sequence.

In some embodiments, the AAV particle genome comprises SEQ ID NO: 2027 (TAU_ITR38) which comprises a 5′ inverted terminal repeat (ITR) sequence region and a 3′ ITR sequence region, a CAG promoter comprising a CMVie region and a minimal CBA promoter region, a human growth hormone-2 signal sequence, a PHF1 antibody heavy chain variable region, a (G4S)3 linker (“(G4S)3” disclosed as SEQ ID NO: 4537), a PHF1 antibody light chain variable region, a His tag, a human growth hormone polyadenylation signal sequence and a human albumin derived filler sequence.

In some embodiments, the AAV particle genome comprises SEQ ID NO: 2028 (TAU_ITR39) which comprises a 5′ inverted terminal repeat (ITR) sequence region and a 3′ ITR sequence region, a CAG promoter comprising a CMVie region and a minimal CBA promoter region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, a human growth hormone polyadenylation signal sequence and a human albumin derived filler sequence.

In some embodiments, the AAV particle genome comprises SEQ ID NO: 2029 (TAU_ITR40) which comprises a 5′ inverted terminal repeat (ITR) sequence region and a 3′ ITR sequence region, a CAG promoter comprising a CMVie region and a minimal CBA promoter region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, a His tag, a human growth hormone polyadenylation signal sequence and a human albumin derived filler sequence.

TABLE 26
Sequence Regions in ITR to ITR Sequences

| | TAU_ITR41 | | TAU_ITR42 | | TAU_ITR43 | | TAU_ITR44 | | TAU_ITR45 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2030) | | 2031) | | 2032) | | 2033) | | 2034) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2077 | 141 | 2077 | 141 | 2077 | 141 | 2077 | 141 | 2077 | 141 |
| CAG | 2080 | 1715 | 2080 | 1715 | 2080 | 1715 | 2080 | 1715 | 2080 | 1715 |
| promoter | | | | | | | | | | |
| CMVie | 2081 | 299 | 2081 | 299 | 2081 | 299 | 2081 | 299 | 2081 | 299 |
| CBA min. | 2082 | 283 | 2082 | 283 | 2082 | 283 | 2082 | 283 | 2082 | 283 |
| Signal | 2016 | 84 | 2106 | 84 | 1862 | 57 | 1862 | 57 | 1862 | 57 |
| VL | 1957 | 339 | 1957 | 339 | 1957 | 339 | 1957 | 339 | 1957 | 339 |
| Linker | 1730 | 45 | 1730 | 45 | 1730 | 45 | 1730 | 45 | 1730 | 45 |
| VH | 1839 | 351 | 1839 | 351 | 1839 | 351 | 1839 | 351 | 1839 | 351 |
| Tag | 2120 | 18 | 2121 | 18 | 2118 | 27 | 2121 | 18 | 2118 | 27 |
| Poly A | 2123 | 447 | 2123 | 477 | 2123 | 477 | 2123 | 477 | 2122 | 127 |
| Filler | 2126 | 1240 | 2126 | 1240 | 2126 | 1240 | 2126 | 1240 | 2125 | 1153 |
| 3′ ITR | 2079 | 141 | 2079 | 141 | 2079 | 141 | 2079 | 141 | 2079 | 141 |

In some embodiments, the AAV particle genome comprises SEQ ID NO: 2030 (TAU_ITR41) which comprises a 5′ inverted terminal repeat (ITR) sequence region and a 3′ ITR sequence region, a CAG promoter comprising a CMVie region and a minimal CBA promoter region, a human growth hormone-2 signal sequence, a PHF1 antibody light chain variable region, a (G4S)3 linker (“(G4S)3” disclosed as SEQ ID NO: 4537), a PHF1 antibody heavy chain variable region, a SEKDEL tag (“SEKDEL” disclosed as SEQ ID NO: 4546), a human growth hormone polyadenylation signal sequence and a human albumin derived filler sequence.

In some embodiments, the AAV particle genome comprises SEQ ID NO: 2031 (TAU_ITR42) which comprises a 5′ inverted terminal repeat (ITR) sequence region and a 3′ ITR sequence region, a CAG promoter comprising a CMVie region and a minimal CBA promoter region, a human growth hormone-2 signal sequence, a PHF1 antibody light chain variable region, a (G4S)3 linker (“(G4S)3” disclosed as SEQ ID NO: 4537), a PHF1 antibody heavy chain variable region, a His tag, a human growth hormone polyadenylation signal sequence and a human albumin derived filler sequence.

In some embodiments, the AAV particle genome comprises SEQ ID NO: 2032 (TAU_ITR43) which comprises a 5′ inverted terminal repeat (ITR) sequence region and a 3′ ITR sequence region, a CAG promoter comprising a CMVie region and a minimal CBA promoter region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, a human growth hormone polyadenylation signal sequence and a human albumin derived filler sequence.

In some embodiments, the AAV particle genome comprises SEQ ID NO: 2033 (TAU_ITR44) which comprises a 5′ inverted terminal repeat (ITR) sequence region and a 3′ ITR sequence region, a CAG promoter comprising a CMVie region and a minimal CBA promoter region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, a His tag, a human growth hormone polyadenylation signal sequence and a human albumin derived filler sequence.

In some embodiments, the AAV particle genome comprises SEQ ID NO: 2034 (TAU_ITR45) which comprises a 5′ inverted terminal repeat (ITR) sequence region and a 3′ ITR sequence region, a CAG promoter comprising a CMVie region and a minimal CBA promoter region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, a rabbit globin polyadenylation signal sequence and a human albumin derived filler sequence.

TABLE 27
Sequence Regions in ITR to ITR Sequences

| | TAU_ITR46 | | TAU_ITR47 | | TAU_ITR48 | | TAU_ITR49 | | TAU_ITR50 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2035) | | 2036) | | 2037) | | 2038) | | 2039) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2077 | 141 | 2077 | 141 | 2077 | 141 | 2077 | 141 | 2077 | 141 |
| CBA | 2084 | 654 | 2084 | 654 | 2084 | 654 | 2084 | 654 | 2084 | 654 |
| promoter | | | | | | | | | | |
| Exon | 2090 | 134 | 2092 | 59 | 2091 | 102 | 2090 | 134 | 2090 | 134 |
| Intron | 2095 | 32 | 2095 | 32 | 2096 | 15 | 2096 | 15 | 2095 | 32 |
| Intron | 2099 | 73 | 2098 | 168 | 2102 | 53 | 2102 | 53 | 2100 | 73 |
| Exon | 2093 | 53 | 2093 | 53 | 2093 | 53 | 2093 | 53 | 2093 | 53 |
| Signal | 1741 | 57 | 1741 | 57 | 1741 | 57 | 1741 | 57 | 1741 | 57 |
| VH | 1839 | 351 | 1839 | 351 | 1839 | 351 | 1839 | 351 | 1839 | 351 |
| Linker | 1730 | 45 | 1730 | 45 | 1730 | 45 | 1730 | 45 | 1730 | 45 |
| VL | 1957 | 339 | 1957 | 339 | 1957 | 339 | 1957 | 339 | 1957 | 339 |
| Tag | 2118 | 27 | 2118 | 27 | 2118 | 27 | 2118 | 27 | 2118 | 27 |
| Poly A | 2122 | 127 | 2122 | 127 | 2122 | 127 | 2122 | 127 | 2122 | 127 |
| 3′ ITR | 2079 | 141 | 2079 | 141 | 2079 | 141 | 2079 | 141 | 2079 | 141 |

In some embodiments, the AAV particle genome comprises SEQ ID NO: 2035 (TAU_ITR46) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

In some embodiments, the AAV particle genome comprises SEQ ID NO: 2036 (TAU_ITR47) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

In some embodiments, the AAV particle genome comprises SEQ ID NO: 2037 (TAU_ITR48) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

In some embodiments, the AAV particle genome comprises SEQ ID NO: 2038 (TAU_ITR49) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

In some embodiments, the AAV particle genome comprises SEQ ID NO: 2039 (TAU_ITR50) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

TABLE 28
Sequence Regions in ITR to ITR Sequences

| | TAU_ITR51 | | TAU_ITR52 | | TAU_ITR53 | | TAU_ITR54 | | TAU_ITR55 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2040) | | 2041) | | 2042) | | 2043) | | 2044) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2077 | 141 | 2077 | 141 | 2077 | 141 | 2077 | 141 | 2077 | 141 |
| CBA | 2084 | 654 | 2084 | 654 | 2084 | 654 | 2084 | 654 | 2084 | 654 |
| promoter | | | | | | | | | | |
| Exon | 2092 | 59 | 2090 | 134 | 2090 | 134 | — | — | — | — |
| Intron | 2095 | 32 | 2095 | 32 | 2095 | 32 | — | — | 2103 | 172 |
| Intron | 2098 | 168 | 2097 | 347 | 2097 | 347 | — | — | — | — |
| Exon | 2093 | 53 | 2093 | 53 | 2093 | 53 | — | — | — | — |
| Signal | 1862 | 57 | 1862 | 57 | 1862 | 57 | 1862 | 57 | 1862 | 57 |
| VL | 1957 | 339 | 1957 | 339 | 1957 | 339 | 1957 | 339 | 1957 | 339 |
| Linker | 1730 | 45 | 1730 | 45 | 1730 | 45 | 1730 | 45 | 1736 | 45 |
| VH | 1839 | 351 | 1839 | 351 | 1839 | 351 | 1839 | 351 | 1839 | 351 |
| Tag | 2118 | 27 | 2118 | 27 | 2118 | 27 | 2118 | 27 | 2118 | 27 |
| Poly A | 2122 | 127 | 2122 | 127 | 2122 | 127 | 2122 | 127 | 2122 | 127 |
| Filler | — | — | — | — | 2125 | 1153 | — | — | — | — |
| 3′ ITR | 2079 | 141 | 2079 | 141 | 2079 | 141 | 2079 | 141 | 2079 | 141 |

In some embodiments, the AAV particle genome comprises SEQ ID NO: 2040 (TAU_ITR51) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, and a rabbit globin polyadenylation signal sequence.

In some embodiments, the AAV particle genome comprises SEQ ID NO: 2041 (TAU_ITR52) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, and a rabbit globin polyadenylation signal sequence.

In some embodiments, the AAV particle genome comprises SEQ ID NO: 2042 (TAU_ITR53) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, a rabbit globin polyadenylation signal sequence and a human albumin derived filler sequence.

In some embodiments, the AAV particle genome comprises SEQ ID NO: 2043 (TAU_ITR54) which comprises a 5′ inverted terminal repeat (ITR) sequence region and a 3′ ITR sequence region, a CBA promoter, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, and a rabbit globin polyadenylation signal sequence.

In some embodiments, the AAV particle genome comprises SEQ ID NO: 2044 (TAU_ITR55) which comprises a 5′ inverted terminal repeat (ITR) sequence region and a 3′ ITR sequence region, a CBA promoter, an SV40 intron, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, and a rabbit globin polyadenylation signal sequence.

TABLE 29
Sequence Regions in ITR to ITR Sequences

| | TAU_ITR56 | | TAU_ITR57 | | TAU_ITR58 | | TAU_ITR59 | | TAU_ITR60 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2045) | | 2046) | | 2047) | | 2048) | | 2049) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2077 | 141 | 2077 | 141 | 2077 | 141 | 2077 | 141 | 2077 | 141 |
| CBA | 2084 | 654 | 2084 | 654 | 2084 | 654 | 2084 | 654 | 2084 | 654 |
| promoter | | | | | | | | | | |
| Exon | 2090 | 134 | 2090 | 134 | 2090 | 134 | — | — | — | — |
| Intron | 2095 | 32 | 2095 | 32 | 2095 | 32 | — | — | 2103 | 172 |
| Intron | 2097 | 347 | 2097 | 347 | 2101 | 73 | — | — | — | — |
| Exon | 2093 | 53 | 2093 | 53 | 2093 | 53 | — | — | — | — |
| Signal | 1741 | 57 | 1741 | 57 | 1741 | 57 | 1741 | 57 | 1741 | 57 |
| VH | 1839 | 351 | 1839 | 351 | 1839 | 351 | 1839 | 351 | 1839 | 351 |
| Linker | 1730 | 45 | 1730 | 45 | 1730 | 45 | 1730 | 45 | 1730 | 45 |
| VL | 1957 | 339 | 1957 | 339 | 1957 | 339 | 1957 | 339 | 1957 | 339 |
| Tag | 2118 | 27 | 2118 | 27 | 2118 | 27 | 2118 | 27 | 2118 | 27 |
| Poly A | 2122 | 127 | 2122 | 127 | 2122 | 127 | 2122 | 127 | 2122 | 127 |
| Filler | 2125 | 1153 | — | — | — | — | — | — | — | — |
| 3′ ITR | 2079 | 141 | 2079 | 141 | 2079 | 141 | 2079 | 141 | 2079 | 141 |

In some embodiments, the AAV particle genome comprises SEQ ID NO: 2045 (TAU_ITR56) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, a rabbit globin polyadenylation signal sequence and a human albumin derived filler sequence.

In some embodiments, the AAV particle genome comprises SEQ ID NO: 2046 (TAU_ITR57) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

In some embodiments, the AAV particle genome comprises SEQ ID NO: 2047 (TAU_ITR58) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF11 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

In some embodiments, the AAV particle genome comprises SEQ ID NO: 2048 (TAU_ITR59) which comprises a 5′ inverted terminal repeat (ITR) sequence region and a 3′ ITR sequence region, a CBA promoter, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

In some embodiments, the AAV particle genome comprises SEQ ID NO: 2049 (TAU_ITR60) which comprises a 5′ inverted terminal repeat (ITR) sequence region and a 3′ ITR sequence region, a CBA promoter, an SV40 intron, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, and a rabbit globin polyadenylation signal sequence.

TABLE 30
Sequence Regions in ITR to ITR Sequences

| | TAU_ITR61 | | TAU_ITR62 | | TAU_ITR63 | | TAU_ITR64 | |
|---|---|---|---|---|---|---|---|---|
| | (SEQ ID NO: | | (SEQ ID NO: | | (SEQ ID NO: | | (SEQ ID NO: | |
| | 2050) | | 2051) | | 2052) | | 2053) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2077 | 141 | 2077 | 141 | 2077 | 141 | 2077 | 141 |
| CBA | 2084 | 654 | 2084 | 654 | 2084 | 654 | 2084 | 654 |
| promoter | | | | | | | | |
| Exon | 2090 | 134 | 2090 | 134 | 2090 | 134 | 2090 | 134 |
| Intron | 2095 | 32 | 2095 | 32 | 2095 | 32 | 2095 | 32 |
| Intron | 2097 | 347 | 2097 | 347 | 2097 | 347 | 2097 | 347 |
| Exon | 2093 | 53 | 2093 | 53 | 2093 | 53 | 2093 | 53 |
| Signal | 1741 | 57 | 1741 | 57 | 1741 | 57 | 1741 | 57 |
| VH1 | 1839 | 351 | 1839 | 351 | 1839 | 351 | 1839 | 351 |
| Linker | 1730 | 45 | 1730 | 45 | 1730 | 45 | 1730 | 45 |
| VL | 1957 | 339 | 1957 | 339 | 1957 | 339 | 1957 | 339 |
| Tag | — | — | — | — | 2118 | 27 | 2118 | 27 |
| Linker | — | — | — | — | 1724 | 12 | 1724 | 12 |
| Linker | 1729 | 18 | 1729 | 18 | 1726 | 54 | 1726 | 54 |
| Signal | 1741 | 57 | 1741 | 57 | 1741 | 57 | 1741 | 57 |
| VH | 1839 | 351 | 1839 | 351 | 1839 | 351 | 1839 | 351 |
| Linker | 1730 | 45 | 1730 | 45 | 1730 | 45 | 1730 | 45 |
| VL | 1957 | 339 | 1957 | 339 | 1957 | 339 | 1957 | 339 |
| Tag | — | — | — | — | 2118 | 27 | — | — |
| Linker | — | — | — | — | 1724 | 12 | — | — |
| Linker | 1729 | 18 | — | — | 1726 | 54 | — | — |
| Signal | — | — | — | — | 1741 | 57 | — | — |
| VH | 1839 | 351 | — | — | 1839 | 351 | — | — |
| Linker | 1730 | 45 | — | — | 1730 | 45 | — | — |
| VL | 1957 | 339 | — | — | 1957 | 339 | — | — |
| Tag | 2118 | 27 | 2118 | 27 | 2118 | 27 | 2118 | 27 |
| Poly A | 2122 | 127 | 2122 | 127 | 2122 | 127 | 2122 | 127 |
| 3′ ITR | 2079 | 141 | 2079 | 141 | 2079 | 141 | 2079 | 141 |

In some embodiments, the AAV particle genome comprises SEQ ID NO: 2050 (TAU_ITR61) which comprises a 5′ inverted terminal repeat (ITR) sequence region and a 3′ ITR sequence region, a CBA promotcr, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, a G4S linker (“G4S” disclosed as SEQ ID NO: 4535), a second PHF1 antibody heavy chain signal sequence, a second PHF1 antibody heavy chain variable region, a (G4S)3 linker (“(G4S)3” disclosed as SEQ ID NO: 4537), a second PHF1 antibody light chain variable region, a G4S linker (“G4S” disclosed as SEQ ID NO: 4535), a third PHF1 antibody heavy chain variable region, a (G4S)3 linker (“(G4S)3” disclosed as SEQ ID NO: 4537), a third PHF1 antibody light chain variable region, an HA tag, and a rabbit globin polyadenylation signal sequence.

In some embodiments, the AAV particle genome comprises SEQ ID NO: 2051 (TAU_ITR62) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, a G4S linker (“G4S” disclosed as SEQ ID NO: 4535), a second PHF1 antibody heavy chain signal sequence, a second PHF1 antibody heavy chain variable region, a (G4S)3 linker (“(G4S)3” disclosed as SEQ ID NO: 4537), a second PHF1 antibody light chain variable region, an HA tag, and a rabbit globin polyadenylation signal sequence.

In some embodiments, the AAV particle genome comprises SEQ ID NO: 2052 (TAU_ITR63) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, a furin cleavage site, a T2A linker, a second PHF1 antibody heavy chain signal sequence, a second PHF1 antibody heavy chain variable region, a (G4S)3 linker (“(G4S)3” disclosed as SEQ ID NO: 4537), a second PHF1 antibody light chain variable region, a second HA tag, a second furin cleavage site, a second T2A linker, a third PHF1 antibody heavy chain signal sequence, a third PHF1 antibody heavy chain variable region, a (G4S)3 linker (“(G4S)3” disclosed as SEQ ID NO: 4537), a third PHF1 antibody light chain variable region, a third HA tag, and a rabbit globin polyadenylation signal sequence.

In some embodiments, the AAV particle genome comprises SEQ ID NO: 2053 (TAU_ITR64) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody heavy chain signal sequence, a PHF1 antibody heavy chain variable region, a (G4S)3 linker (“(G4S)3” disclosed as SEQ ID NO: 4537), a PHF1 antibody light chain variable region, an HA tag, a furin cleavage site, a T2A linker, a second PHF1 antibody heavy chain signal sequence, a second PHF1 antibody heavy chain variable region, a (G4S)3 linker (“(G4S)3” disclosed as SEQ ID NO: 4537), a second PHF1 antibody light chain variable region, a second HA tag, and a rabbit globin polyadenylation signal sequence.

TABLE 31
Sequence Regions in ITR to ITR Sequences

| | TAU_ITR65 | | TAU_ITR66 | | TAU_ITR67 | | TAU_ITR68 | |
|---|---|---|---|---|---|---|---|---|
| | (SEQ ID NO: | | (SEQ ID NO: | | (SEQ ID NO: | | (SEQ ID NO: | |
| | 2054) | | 2055) | | 2056) | | 2057) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2077 | 141 | 2077 | 141 | 2077 | 141 | 2077 | 141 |
| CBA | 2084 | 654 | 2084 | 654 | 2084 | 654 | 2084 | 654 |
| promoter | | | | | | | | |
| Exon | 2090 | 134 | 2090 | 134 | 2090 | 134 | 2090 | 134 |
| Intron | 2095 | 32 | 2095 | 32 | 2095 | 32 | 2095 | 32 |
| Intron | 2097 | 347 | 2097 | 347 | 2097 | 347 | 2097 | 347 |
| Exon | 2093 | 53 | 2093 | 53 | 2093 | 53 | 2093 | 53 |
| Signal | 1862 | 57 | 1862 | 57 | 1862 | 57 | 1862 | 57 |
| VL | 1956 | 339 | 1956 | 339 | 1956 | 339 | 1956 | 339 |
| Linker | 1730 | 45 | 1730 | 45 | 1730 | 45 | 1730 | 45 |
| VH | 1839 | 351 | 1839 | 351 | 1839 | 351 | 1839 | 351 |
| Tag | — | — | — | — | 2118 | 27 | 2118 | 27 |
| Linker | — | — | — | — | 1724 | 12 | 1724 | 12 |
| Linker | 1729 | 18 | — | — | 1726 | 54 | 1726 | 54 |
| Signal | — | — | — | — | 1862 | 57 | 1862 | 57 |
| VL | 1956 | 339 | 1956 | 339 | 1956 | 339 | 1956 | 339 |
| Linker | 1730 | 45 | 1730 | 45 | 1730 | 45 | 1730 | 45 |
| VH | 1839 | 351 | 1839 | 351 | 1839 | 351 | 1839 | 351 |
| Tag | — | — | — | — | 2118 | 27 | — | — |
| Linker | — | — | — | — | 1724 | 12 | — | — |
| Linker | 1729 | 18 | | — | 1726 | 54 | — | — |
| Signal | — | — | — | — | 1862 | 57 | — | — |
| VL | 1956 | 339 | — | — | 1956 | 339 | — | — |
| Linker | 1730 | 45 | — | — | 1730 | 45 | — | — |
| VH | 1839 | 351 | — | — | 1839 | 351 | — | — |
| Tag | 2118 | 27 | 2118 | — | 2118 | 27 | 2118 | 27 |
| Poly A | 2122 | 127 | 2122 | 127 | 2122 | 127 | 2122 | 127 |
| 3′ ITR | 2079 | 141 | 2079 | 141 | 2079 | 141 | 2079 | 141 |

In some embodiments, the AAV particle genome comprises SEQ ID NO: 2054 (TAU_ITR65) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody light chain signal sequence, a PHF1 antibody light chain variable region, a (G4S).3 linker (“(G4S)3” disclosed as SEQ ID NO: 4537), a PHF1 antibody heavy chain variable region, a G4S linker (“G4S” disclosed as SEQ ID NO: 4535), a second PHF1 antibody light chain variable region, a (G4S)3 linker (“(G4S)3” disclosed as SEQ ID NO: 4537), a second PHF1 antibody heavy chain variable region, a G4S linker (“G4S” disclosed as SEQ ID NO: 4535), a third PHF1 antibody light chain variable region, a (G4S)3 linker (“(G4S)3” disclosed as SEQ ID NO: 4537), a third PHF1 antibody heavy chain variable region, an HA tag, and a rabbit globin polyadenylation signal sequence.

In some embodiments, the AAV particle genome comprises SEQ ID NO: 2055 (TAU_ITR66) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, a second PHF1 antibody light chain variable region, a (G4S)3 linker (“(G4S)3” disclosed as SEQ ID NO: 4537), a second PHF1 antibody heavy chain variable region, an HA tag, and a rabbit globin polyadenylation signal sequence.

In some embodiments, the AAV particle genome comprises SEQ ID NO: 2056 (TAU_ITR67) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, a furin cleavage site, a T2A linker, a second PHF1 antibody light chain signal sequence, a second PHF1 antibody light chain variable region, a (G4S)3 linker (“(G4S)3” disclosed as SEQ ID NO: 4537), a second PHF1 antibody heavy chain variable region, a second HA tag, a second furin cleavage site, a second T2A linker, a third PHF1 antibody light chain signal sequence, a third PHF1 antibody light chain variable region, a (G4S)3 linker (“(G4S)3” disclosed as SEQ ID NO: 4537), a third PHF1 antibody heavy chain variable region, a third HA tag, and a rabbit globin polyadenylation signal sequence.

In some embodiments, the AAV particle genome comprises SEQ ID NO: 2057 (TAU_ITR68) which comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, a PHF1 antibody light chain signal sequence, a PHF1 antibody light chain variable region, a (G4S)3 linker (“(G4S)3” disclosed as SEQ ID NO: 4537), a PHF1 antibody heavy chain variable region, an HA tag, a furin cleavage site, a T2A linker, a second PHF1 antibody light chain signal sequence, a second PHF1 antibody light chain variable region, a (G4S)3 linker (“(G4S)3” disclosed as SEQ ID NO: 4537), a second PHF1 antibody heavy chain variable region, a second HA tag, and a rabbit globin polyadenylation signal sequence.

TABLE 32
Sequence Regions in ITR to ITR Sequences

| | TAU_ITR69 | | TAU_ITR70 | | TAU_ITR71 | |
|---|---|---|---|---|---|---|
| | (SEQ ID NO: | | (SEQ ID NO: | | (SEQ ID NO: | |
| | 2058) | | 2059) | | 2060) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| Promoter | 2087 | 382 | 2087 | 382 | 2085 | 699 |
| Promoter | 2083 | 260 | 2083 | 260 | — | — |
| Exon | 2090 | 134 | 2090 | 134 | — | — |
| Intron | 2095 | 32 | 2095 | 32 | 2103 | 172 |
| Intron | 2097 | 347 | 2097 | 347 | — | — |
| Exon | 2093 | 53 | 2093 | 53 | — | — |
| VH | 1838 | 1377 | — | — | — | — |
| VL | — | — | 1962 | 714 | 1962 | 714 |
| Linker | 1727 | 75 | 1727 | 75 | 1727 | 75 |
| VL | 1962 | 714 | — | — | — | — |
| VH | — | — | 1838 | 1377 | 1838 | 1377 |
| Poly A | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2058 (TAU_ITR69) which comprises a 5′ inverted terminal repeat (ITR) sequence region and a 3′ ITR sequence region, a CMVie region, a CB promoter, an ie1 exon 1 region, an ie1 intron 1 region, a human beta-globin intron region, a human beta-globin exon region, a PHF1 antibody heavy chain region, an F2A linker, a PHF1 antibody light chain region, and a rabbit globin polyadenylation signal sequence.

In some embodiments, the AAV particle genome comprises SEQ ID NO: 2059 (TAU_ITR70) which comprises a 5′ inverted terminal repeat (ITR) sequence region and a 3′ ITR sequence region, a CMVie region, a CB promoter, an ie1 exon region, an ie1 intron 1 region, a human beta-globin intron region, a human beta-globin exon region, a PHF1 antibody light chain variable region, an F2A linker, a PHF1 antibody heavy chain variable region, and a rabbit globin polyadenylation signal sequence.

In some embodiments, the AAV particle genome comprises SEQ ID NO: 2060 (TAU_ITR71) which comprises a 5′ inverted terminal repeat (ITR) sequence region and a 3′ ITR sequence region, a GFAP promoter, an SV40 intron, a PHF1 antibody light chain variable region, an F2A linker, a PHF1 antibody heavy chain variable region, and a rabbit globin polyadenylation signal sequence.

TABLE 33
Sequence Regions in ITR to ITR Sequences

| | TAU_ITR72 | | TAU_ITR73 | | TAU_ITR74 | | TAU_ITR75 | | TAU_ITR76 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2061) | | 2062) | | 2063) | | 2064) | | 2065) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| CMVie | 2087 | 382 | 2087 | 382 | 2087 | 382 | 2087 | 382 | 2087 | 382 |
| Promoter | 2083 | 260 | 2083 | 260 | 2083 | 260 | 2083 | 260 | 2083 | 260 |
| Intron | 2103 | 172 | 2103 | 172 | 2103 | 172 | 2103 | 172 | 2103 | 172 |
| VH | 1832 | 1374 | 1832 | 1374 | 1832 | 1374 | 1832 | 1374 | 1832 | 1374 |
| Linker | 1725 | 12 | 1725 | 12 | — | — | — | — | — | — |
| Linker | 1727 | 75 | 1728 | 66 | 1727 | 75 | 1732 | 609 | 1728 | 66 |
| VL | 1950 | 714 | 1950 | 714 | 1950 | 714 | 1950 | 714 | 1950 | 714 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2061 (TAU_ITR72) which comprises a 5′ inverted terminal repeat (ITR) sequence region and a 3′ ITR sequence region, a CMVie region, a CB promoter, an SV40 intron, an MC1 antibody heavy chain variable region, a furin cleavage site, an F2A linker, an MC1 antibody light chain variable region, and a rabbit globin polyadenylation signal sequence.

In some embodiments, the AAV particle genome comprises SEQ ID NO: 2062 (TAU_ITR73) which comprises a 5′ inverted terminal repeat (ITR) sequence region and a 3′ ITR sequence region, a CMVie region, a CB promoter, an SV40 intron, an MC1 antibody heavy chain variable region, a furin cleavage site, a P2A linker, an MC1 antibody light chain variable region, and a rabbit globin polyadenylation signal sequence.

In some embodiments, the AAV particle genome comprises SEQ ID NO: 2063 (TAU_ITR74) which comprises a 5′ inverted terminal repeat (ITR) sequence region and a 3′ ITR sequence region, a CMVie region, a CB promoter, an SV40 intron, an MC1 antibody heavy chain variable region, an F2A linker, an MC1 antibody light chain variable region, and a rabbit globin polyadenylation signal sequence.

In some embodiments, the AAV particle genome comprises SEQ ID NO: 2064 (TAU_ITR75) which comprises a 5′ inverted terminal repeat (ITR) sequence region and a 3′ ITR sequence region, a CMVie region, a CB promoter, an SV40 intron, an MC1 antibody heavy chain variable region, an IRES linker, an MC1 antibody light chain variable region, and a rabbit globin polyadenylation signal sequence.

In some embodiments, the AAV particle genome comprises SEQ ID NO: 2065 (TAU_ITR76) which comprises a 5′ inverted terminal repeat (ITR) sequence region and a 3′ ITR sequence region, a CMVie region, a CB promoter, an SV40 intron, an MC1 antibody heavy chain variable region, a P2A linker, an MC1 antibody light chain variable region, and a rabbit globin polyadenylation signal sequence.

TABLE 34
Sequence Regions in ITR to ITR Sequences

| | TAU_ITR77 | | TAU_ITR85 | |
|---|---|---|---|---|
| | (SEQ ID NO: 2066) | | (SEQ ID NO: 2074) | |
| Sequence | Region | Region | Region | Region |
| Regions | SEQ ID NO | SEQ ID NO | length | length |
| 5′ ITR | 2076 | 130 | 2076 | 130 |
| CMVie | 2087 | 382 | 2087 | 382 |
| Promoter | 2083 | 260 | 2083 | 260 |
| Intron | 2103 | 172 | 2103 | 172 |
| VH | 1820 | 1377 | 1838 | 1377 |
| Linker | 1725 | 12 | 1725 | 12 |
| Linker | 1727 | 75 | 1727 | 75 |
| VL | 1940 | 714 | 1962 | 714 |
| PolyA | 2122 | 127 | 2122 | 127 |
| 3′ ITR | 2078 | 130 | 2078 | 130 |

In some embodiments, the AAV particle genome comprises SEQ ID NO: 2066 (TAU_ITR77) which comprises a 5 inverted terminal repeat (ITR) sequence region and a 3′ ITR sequence region, a CMVie region, a CB promoter, an SV40 intron, an IPN002 antibody heavy chain variable region, a furin cleavage site, an F2A linker, an IPN002 antibody light chain variable region, and a rabbit globin polyadenylation signal sequence.

In some embodiments, the AAV particle genome comprises SEQ ID NO: 2074 (TAU_ITR85) which comprises a 5′ inverted terminal repeat (ITR) sequence region and a ITR sequence region, a CMVie region, a CB promoter, an SV40 intron, an PHF1 antibody heavy chain variable region, a furin cleavage site, an F2A linker, an PHF1 antibody light chain variable region, and a rabbit globin polyadenylation signal sequence.

TABLE 35
Sequence Regions in ITR to ITR Sequences

| | TAU_ITR78 | | TAU_ITR79 | | TAU_ITR80 | | TAU_ITR81 | | TAU_ITR82 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2067) | | 2068) | | 2069) | | 2070) | | 2071) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| CMVie | 2087 | 382 | 2087 | 382 | 2087 | 382 | 2087 | 382 | 2087 | 382 |
| Promoter | 2083 | 260 | 2083 | 260 | 2083 | 260 | 2083 | 260 | 2083 | 260 |
| SV40 | 2103 | 172 | 2103 | 172 | 2103 | 172 | 2103 | 172 | 2103 | 172 |
| VL | 1950 | 714 | 1950 | 714 | 1950 | 714 | 1950 | 714 | 1950 | 714 |
| Linker | 1725 | 12 | — | — | — | — | — | — | — | — |
| Linker | 1727 | 75 | 1727 | 75 | 1732 | 609 | 1731 | 75 | 1728 | 66 |
| VH | 1832 | 1374 | 1832 | 1374 | 1832 | 1374 | 1832 | 1374 | 1832 | 1374 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2067 (TAU_ITR78) which comprises a 5′ inverted terminal repeat (ITR) sequence region and a 3′ ITR sequence region, a CMVie region, a CB promoter, an SV40 intron, an MC1 antibody light chain variable region, a furin cleavage site, an F2A linker, an MC1 antibody heavy chain variable region, and a rabbit globin polyadenylation signal sequence.

In some embodiments, the AAV particle genome comprises SEQ ID NO: 2068 (TAU_ITR79) which comprises a 5′ inverted terminal repeat (ITR) sequence region and a 3′ ITR sequence region, a CMVie region, a CB promoter, an SV40 intron, an MC1 antibody light chain variable region, an F2A linker, an MC1 antibody heavy chain variable region, and a rabbit globin polyadenylation signal sequence.

In some embodiments, the AAV particle genome comprises SEQ ID NO: 2069 (TAU_ITR80) which comprises a 5′ inverted terminal repeat (ITR) sequence region and a 3′ ITR sequence region, a CMVie region, a CB promoter, an SV40 intron, an MC1 antibody light chain variable region, an IRES linker, an MC1 antibody heavy chain variable region, and a rabbit globin polyadenylation signal sequence.

In some embodiments, the AAV particle genome comprises SEQ ID NO: 2070 (TAU_ITR81) which comprises a 5′ inverted terminal repeat (ITR) sequence region and a 3′ ITR sequence region, a CMVie region, a CB promoter, an SV40 intron, an MC1 antibody light chain variable region, a (G4S)5 linker (“(G4S)5” disclosed as SEQ ID NO: 4538), an MC1 antibody heavy chain variable region, and a rabbit globin polyadenylation signal sequence.

In some embodiments, the AAV particle genome comprises SEQ ID NO: 2071 (TAU_ITR82) which comprises a 5′ inverted terminal repeat (ITR) sequence region and a 3′ ITR sequence region, a CMVie region, a CB promoter, an SV40 intron, an MC1 antibody light chain variable region, an P2A linker, an MC1 antibody heavy chain variable region, and a rabbit globin polyadenylation signal sequence.

TABLE 36
Sequence Regions in ITR to ITR Sequences

| | TAU_ITR83 | | TAU_ITR84 | | TAU_ITR86 | |
|---|---|---|---|---|---|---|
| | (SEQ ID NO: | | (SEQ IDNO: | | (SEQ ID NO: | |
| | 2072) | | 2073) | | 2075) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| CMVie | 2087 | 382 | 2087 | 382 | 2087 | 382 |
| Promoter | 2083 | 260 | 2083 | 260 | 2083 | 260 |
| Intron | 2103 | 172 | 2103 | 172 | 2103 | 172 |
| VL | 1950 | 714 | 1940 | 714 | 1962 | 714 |
| Linker | 1725 | 12 | — | — | — | — |
| Linker | 1728 | 66 | 1727 | 75 | 1727 | 75 |
| VH | 1832 | 1374 | 1820 | 1377 | 1838 | 1377 |
| Poly A | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2072 (TAU_ITR83) which comprises a 5′ inverted terminal repeat (ITR) sequence region and a 3′ ITR sequence region, a CMVie region, a CB promoter, an SV40 intron, an MC1 antibody light chain variable region, a furin cleavage site, a P2A linker, an MC1 antibody heavy chain variable region, and a rabbit globin polyadenylation signal sequence.

In some embodiments, the AAV particle genome comprises SEQ ID NO: 2073 (TAU_ITR84) which comprises a 5′ inverted terminal repeat (ITR) sequence region and a 3′ ITR sequence region, a CMVie region, a CB promoter, an SV40 intron, an IPN002 antibody light chain variable region, an F2A linker, an IPN002 antibody heavy chain variable region, and a rabbit globin polyadenylation signal sequence.

In some embodiments, the AAV particle genome comprises SEQ ID NO: 2075 (TAU_ITR86) which comprises a 5′ inverted terminal repeat (ITR) sequence region and a 3′ ITR sequence region, a CMVie region, a CB promoter, an SV40 intron, a PHF1 antibody light chain variable region, an F2A linker, a PHF1 antibody heavy chain variable region, and a rabbit globin polyadenylation signal sequence.

TABLE 37
Sequence Regions in ITR to ITR Sequences

| | TAU_ITR89 | | TAU_ITR97 | | TAU_ITR218 | | TAU_ITR220 | |
|---|---|---|---|---|---|---|---|---|
| | (SEQ ID NO: | | (SEQ ID NO: | | (SEQ ID NO: | | (SEQ ID NO: | |
| | 2139) | | 2147) | | 2290) | | 2292) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| CAG | 2080 | 1715 | 2080 | 1715 | 2080 | 1715 | 2080 | 1715 |
| promoter | | | | | | | | |
| CMVie | 2081 | 299 | 2081 | 299 | 2081 | 299 | 2081 | 299 |
| CBA min. | 2082 | 283 | 2082 | 283 | 2082 | 283 | 2082 | 283 |
| Signal | 1740 | 57 | 1740 | 57 | 1740 | 57 | 1740 | 57 |
| VH | 1809 | 345 | 1809 | 345 | 1809 | 345 | 1809 | 345 |
| Constant | 1742 | 969 | 1742 | 969 | 2242 | 309 | 2243 | 291 |
| Linker | 1725 | 12 | 1724 | 12 | 1724 | 12 | 1724 | 12 |
| Linker | 1727 | 75 | 1726 | 54 | 1726 | 54 | 1726 | 54 |
| Signal | 1861 | 57 | 1861 | 57 | 1861 | 57 | 1861 | 57 |
| VL | 1926 | 321 | 1926 | 321 | 1926 | 321 | 1926 | 321 |
| Constant | 1864 | 321 | 1864 | 321 | 1864 | 321 | 1864 | 321 |
| Poly A | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2139 (TAU_ITR89) which comprises a 5′ inverted terminal repeat (ITR) sequence region and a 3′ ITR sequence region, a CAG promoter comprising a CMVie region and a minimal CBA promoter region, a heavy chain signal sequence, a C10.2 antibody heavy chain variable region, a heavy chain constant region, a furin cleavage site, a F2A linker, a light chain signal sequence, a C10.2 antibody light chain variable region, a light chain constant region, and a rabbit globin polyadenylation signal sequence.

In some embodiments, the AAV particle genome comprises SEQ ID NO: 2147 (TAU_ITR97) which comprises a 5′ inverted terminal repeat (ITR) sequence region and a 3′ ITR sequence region, a CAG promoter comprising a CMVie region and a minimal CBA promoter region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2290 (TAU_ITR218) which comprises a 5′ inverted terminal repeat (ITR) sequence region and a 3′ ITR sequence region, a CAG promoter comprising a CMVie region and a minimal CBA promoter region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2290 (TAU_ITR220) which comprises a 5′ inverted terminal repeat (ITR) sequence region and a 3′ ITR sequence region, a CAG promoter comprising a CMVie region and a minimal CBA promoter region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

TABLE 38
Sequence Regions in ITR to ITR Sequences

| | TAU_ITR217 | | TAU_ITR126 | |
|---|---|---|---|---|
| | (SEQ ID NO: 2289) | | (SEQ ID NO: 2176) | |
| Sequence | Region SEQ | Region | Region SEQ | Region |
| Regions | ID NO | length | ID NO | length |
| 5′ ITR | 2076 | 130 | 2076 | 130 |
| CAG promoter | 2080 | 1715 | 2080 | 1715 |
| CMVie | 2081 | 299 | 2081 | 299 |
| CBA min. | 2082 | 283 | 2082 | 283 |
| Signal | 1740 | 57 | 1740 | 57 |
| VH | 1809 | 345 | 1821 | 351 |
| Constant | 2242 | 309 | 2242 | 309 |
| Linker | 2244 | 198 | 2244 | 198 |
| Linker | 1724 | 12 | 1724 | 12 |
| Linker | 1726 | 54 | 1726 | 54 |
| Signal | 1861 | 57 | 1861 | 57 |
| VL | 1926 | 321 | 1939 | 336 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 2289 (TAU_ITR217) which comprises a 5′ inverted terminal repeat (ITR) sequence region and a 3′ ITR sequence region, a CAG promoter comprising a CMVie region and a minimal CBA promoter region, a heavy chain signal sequence, a C10.2 antibody heavy chain variable region, a heavy chain constant region, a hinge region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2289 (TAU_ITR217) which comprises a 5′ inverted terminal repeat (ITR) sequence region and a 3′ ITR sequence region, a CAG promoter comprising a CMVie region and a minimal CBA promoter region, a heavy chain signal sequence, a C10.2 antibody heavy chain variable region, a heavy chain constant region, a hinge region, a furin cleavage site, a T2A linker, a light chain signal sequence, a C10.2 antibody light chain variable region, a light chain constant region, and a rabbit globin polyadenylation signal sequence.

TABLE 39
Sequence Regions in ITR to ITR Sequences

| | TAU_ITR222 | | TAU_ITR223 | | TAU_ITR224 | | TAU_ITR221 | | TAU_ITR151 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2294) | | 2295) | | 2296) | | 2293) | | 2201) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| CAG | 2080 | 1715 | 2080 | 1715 | 2080 | 1715 | 2080 | 1715 | 2080 | 1715 |
| promoter | | | | | | | | | | |
| CMVie | 2081 | 299 | 2081 | 299 | 2081 | 299 | 2081 | 299 | 2081 | 299 |
| CBA min. | 2082 | 283 | 2082 | 283 | 2082 | 283 | 2082 | 283 | 2082 | 283 |
| Signal | 1861 | 57 | 1861 | 57 | 1861 | 57 | 1861 | 57 | 1861 | 57 |
| VL | 1926 | 321 | 1926 | 321 | 1926 | 321 | 1926 | 321 | 1939 | 336 |
| Linker | 2247 | 45 | 2250 | 75 | 2253 | 120 | 2245 | 15 | 2246 | 30 |
| VH | 1809 | 345 | 1809 | 345 | 1809 | 345 | 1809 | 345 | 1821 | 351 |
| Tag | 2118 | 27 | 2118 | 27 | 2118 | 27 | 2118 | 27 | 2118 | 27 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2294 (TAU_ITR222) which comprises a 5′ inverted terminal repeat (ITR) sequence region and a 3′ ITR sequence region, a CAG promoter comprising a CMVie region and a minimal CBA promoter region, a light chain signal sequence, a C10.2 antibody light chain variable region, a (G4S)3 linker (“(G4S)3” disclosed as SEQ ID NO: 4537), a C10.2 antibody heavy chain variable region, an HA tag, and a rabbit globin polyadenylation signal sequence.

In some embodiments, the AAV particle genome comprises SEQ ID NO: 2295 (TAU_ITR223) which comprises a 5′ inverted terminal repeat (ITR) sequence region and a 3′ ITR sequence region, a CAG promoter comprising a CMVie region and a minimal CBA promoter region, a light chain signal sequence, a C10.2 antibody light chain variable region, a (G4S)3 linker (“(G4S)3” disclosed as SEQ ID NO: 4537), a C10.2 antibody heavy chain variable region, an HA tag, and a rabbit globin polyadenylation signal sequence.

In some embodiments, the AAV particle genome comprises SEQ ID NO: 2296 (TAU_ITR224) which comprises a 5′ inverted terminal repeat (ITR) sequence region and a 3′ ITR sequence region, a CAG promoter comprising a CMVie region and a minimal CBA promoter region, a light chain signal sequence, a C10.2 antibody light chain variable region, a (G4S)8 linker (“(G4S)8” disclosed as SEQ ID NO: 4542), a C10.2 antibody heavy chain variable region, an HA tag, and a rabbit globin polyadenylation signal sequence.

In some embodiments, the AAV particle genome comprises SEQ ID NO: 2293 (TAU_ITR221) which comprises a 5′ inverted terminal repeat (ITR) sequence region and a 3′ ITR sequence region, a CAG promoter comprising a CMVie region and a minimal CBA promoter region, a light chain signal sequence, a C10.2 antibody light chain variable region, a G4S linker (“G4S” disclosed as SEQ ID NO: 4535), a C10.2 antibody heavy chain variable region, an HA tag, and a rabbit globin polyadenylation signal sequence.

In some embodiments, the AAV particle genome comprises SEQ ID NO: 2201 (TAU_ITR151) which comprises a 5′ inverted terminal repeat (ITR) sequence region and a 3′ ITR sequence region, a CAG promoter comprising a CMVie region and a minimal CBA promoter region, a light chain signal sequence, an IPN002 antibody light chain variable region, a (G4S)2 linker (“(G4S)2” disclosed as SEQ ID NO: 4539), an IPN002 antibody heavy chain variable region, an HA tag, and a rabbit globin polyadenylation signal sequence.

TABLE 40
Sequence Regions in ITR to ITR Sequences

| | TAU_ITR93 | | TAU_ITR100 | | TAU_ITR219 | | TAU_ITR130 | |
|---|---|---|---|---|---|---|---|---|
| | (SEQ ID NO: | | (SEQ ID NO: | | (SEQ ID NO: | | (SEQ ID NO: | |
| | 2143) | | 2150) | | 2291) | | 2180) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| CAG | 2080 | 1715 | 2080 | 1715 | 2080 | 1715 | 2080 | 1715 |
| promoter | | | | | | | | |
| CMVie | 2081 | 299 | 2081 | 299 | 2081 | 299 | 2081 | 299 |
| CBA mm. | 2082 | 283 | 2082 | 283 | 2082 | 283 | 2082 | 283 |
| Signal | 1861 | 57 | 1861 | 57 | 1861 | 57 | 1861 | 57 |
| VL | 1926 | 321 | 1926 | 321 | 1926 | 321 | 1939 | 336 |
| Constant | 1864 | 321 | 1864 | 321 | 1864 | 321 | 1864 | 321 |
| Linker | 1727 | 75 | 1726 | 54 | 1726 | 54 | 1726 | 54 |
| Signal | 1740 | 57 | 1740 | 57 | 1740 | 57 | 1740 | 57 |
| VH | 1809 | 345 | 1809 | 345 | 1809 | 345 | 1821 | 351 |
| Constant | 1742 | 969 | 1743 | 972 | 2242 | 309 | 2242 | 309 |
| Poly A | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2143 (TAU_ITR93) which comprises a 5′ inverted terminal repeat (ITR) sequence region and a 3′ ITR sequence region, a CAG promoter comprising a CMVie region and a minimal CBA promoter region, a light chain signal sequence, a C10.2 antibody light chain variable region, a light chain constant region, a F2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2150 (TAU_ITR100) which comprises a 5′ inverted terminal repeat (ITR) sequence region and a 3′ ITR sequence region, a CAG promoter comprising a CMVie region and a minimal CBA promoter region, a light chain signal sequence, a C10.2 antibody light chain variable region, a light chain constant region, a T2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2291 (TAU_ITR219) which comprises a 5′ inverted terminal repeat (ITR) sequence region and a 3′ ITR sequence region, a CAG promoter comprising a CMVie region and a minimal CBA promoter region, a light chain signal sequence, a C10.2 antibody light chain variable region, a light chain constant region, a T2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2180 (TAU_ITR130) which comprises a 5′ inverted terminal repeat (ITR) sequence region and a 3′ ITR sequence region, a CAG promoter comprising a CMVie region and a minimal CBA promoter region, a light chain signal sequence, an IPN002 antibody light chain variable region, a light chain constant region, a T2A linker, a heavy chain signal sequence, an IPN002 antibody heavy chain variable region, a heavy chain constant region, and a rabbit globin polyadenylation signal sequence.

TABLE 41
Sequence Regions in ITR to ITR Sequences

| | TAU_ITR90 | | TAU_ITR98 | | TAU_ITR226 | | TAU_ITR228 | | TAU_ITR198 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2140) | | 2148) | | 2298) | | 2300) | | 2270) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2085 | 699 | 2085 | 699 | 2085 | 699 | 2085 | 699 | 2085 | 699 |
| Intron | 2240 | 566 | 2240 | 566 | 2240 | 566 | 2240 | 566 | 2240 | 566 |
| Signal | 1740 | 57 | 1740 | 57 | 1740 | 57 | 1740 | 57 | 1740 | 57 |
| VH | 1809 | 345 | 1809 | 345 | 1809 | 345 | 1809 | 345 | 1821 | 351 |
| Constant | 1742 | 969 | 1742 | 969 | 2242 | 309 | 2243 | 291 | 2242 | 309 |
| Linker | 1725 | 12 | 1724 | 12 | 1724 | 12 | 1724 | 12 | 1724 | 12 |
| Linker | 1727 | 75 | 1726 | 54 | 1726 | 54 | 1726 | 54 | 1726 | 54 |
| Signal | 1861 | 57 | 1861 | 57 | 1861 | 57 | 1861 | 57 | 1861 | 57 |
| VL | 1926 | 321 | 1926 | 321 | 1926 | 321 | 1926 | 321 | 1939 | 336 |
| Constant | 1864 | 321 | 1864 | 321 | 1864 | 321 | 1864 | 321 | 1864 | 321 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2140 (TAU_ITR90) which comprises a 5′ inverted terminal repeat (ITR) sequence region and a 3′ ITR sequence region, a GFAP promoter, a human beta-globin intron region, a heavy chain signal sequence, a C10.2 antibody heavy chain variable region, a heavy chain constant region, a furin cleavage site, a F2A linker, a light chain signal sequence, a C10.2 antibody light chain variable region, a light chain constant region, and a rabbit globin polyadenylation signal sequence.

In some embodiments, the AAV particle genome comprises SEQ ID NO: 2148 (TAU_ITR98) which comprises a 5′ inverted terminal repeat (ITR) sequence region and a 3′ ITR sequence region, a GFAP promoter, a human beta-globin intron region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2298 (TAU_ITR226) which comprises a 5′ inverted terminal repeat (ITR) sequence region and a 3′ ITR sequence region, a GFAP promoter, a human beta-globin intron region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2300 (TAU_ITR228) which comprises a 5′ inverted terminal repeat (ITR) sequence region and a 3′ ITR sequence region, a GFAP promoter, a human beta-globin intron region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2270 (TAU_ITR198) which comprises a 5′ inverted terminal repeat (ITR) sequence region and a 3′ ITR sequence region, a GFAP promoter, a human beta-globin intron region, a heavy chain signal sequence, an IPN002 antibody heavy chain variable region, a heavy chain constant region, a furin cleavage site, a T2A linker, a light chain signal sequence, an IPN002 antibody light chain variable region, a light chain constant region, and a rabbit globin polyadenylation signal sequence.

TABLE 42
Sequence Regions in ITR to ITR Sequences

| | TAU_ITR225 | | TAU_ITR197 | | TAU_ITR207 | | TAU_ITR122 | |
|---|---|---|---|---|---|---|---|---|
| | (SEQ ID NO: | | (SEQ ID NO: | | (SEQ ID NO: | | (SEQ ID NO: | |
| | 2297) | | 2269) | | 2279) | | 2172) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2085 | 699 | 2085 | 699 | 2086 | 557 | 2239 | 380 |
| Intron | 2240 | 566 | 2240 | 566 | 2240 | 566 | 2240 | 566 |
| Signal | 1740 | 57 | 1740 | 57 | 1740 | 57 | 2241 | 57 |
| VH | 1809 | 345 | 1821 | 351 | 1821 | 351 | 1821 | 351 |
| Constant | 2242 | 309 | 2242 | 309 | 2242 | 309 | 2242 | 309 |
| Linker | 2244 | 198 | 2244 | 198 | 2244 | 198 | 2244 | 198 |
| Linker | 1724 | 12 | 1724 | 12 | 1724 | 12 | 1724 | 12 |
| Linker | 1726 | 54 | 1726 | 54 | 1726 | 54 | 1726 | 54 |
| Signal | 1861 | 57 | 1861 | 57 | 1861 | 57 | 1861 | 57 |
| VL | 1926 | 321 | 1939 | 336 | 1939 | 336 | 1939 | 336 |
| Constant | 1864 | 321 | 1864 | 321 | 1864 | 321 | 1864 | 321 |
| Poly A | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2297 (TAU_ITR225) which comprises a 5′ inverted terminal repeat (ITR) sequence region and a 3′ ITR sequence region, a GFAP promoter, a human beta-globin intron region, a heavy chain signal sequence, a C10.2 antibody heavy chain variable region, a heavy chain constant region, a hinge region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2269 (TAU_ITR197) which comprises a 5′ inverted terminal repeat (ITR) sequence region and a 3′ ITR sequence region, a GFAP promoter, a human beta-globin intron region, a heavy chain signal sequence, an IPN002 antibody heavy chain variable region, a heavy chain constant region, a hinge region, a furin cleavage site, a T2A linker, a light chain signal sequence, an IPN002 antibody light chain variable region, a light chain constant region, and a rabbit globin polyadenylation signal sequence.

In some embodiments, the AAV particle genome comprises SEQ ID NO: 2279 (TAU_ITR207) which comprises a 5′ inverted terminal repeat (ITR) sequence region and a 3′ ITR sequence region, a hSYN promoter, a human beta-globin intron region, a heavy chain signal sequence, an IPN002 antibody heavy chain variable region, a heavy chain constant region, a hinge region, a furin cleavage site, a T2A linker, a light chain signal sequence, an IPN002 antibody light chain variable region, a light chain constant region, and a rabbit globin polyadenylation signal sequence.

In some embodiments, the AAV particle genome comprises SEQ ID NO: 2172 (TAU_ITR122) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a heavy chain constant region, a hinge region, a furin cleavage site, a T2A linker, a light chain signal sequence, an IPN002 antibody light chain variable region, a light chain constant region, and a rabbit globin polyadenylation signal sequence.

TABLE 43
Sequence Regions in ITR to ITR Sequences

| | TAU_ITR230 | | TAU_ITR231 | | TAU_ITR232 | | TAU_ITR204 | | TAU_ITR205 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2302) | | 2303) | | 2304) | | 2276) | | 2277) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2085 | 699 | 2085 | 699 | 2085 | 699 | 2085 | 699 | 2085 | 699 |
| Intron | 2240 | 566 | 2240 | 566 | 2240 | 566 | 2240 | 566 | 2240 | 566 |
| Signal | 1861 | 57 | 1861 | 57 | 1861 | 57 | 1861 | 57 | 1861 | 57 |
| VL | 1926 | 321 | 1926 | 321 | 1926 | 321 | 1939 | 336 | 1939 | 336 |
| Linker | 2247 | 45 | 2250 | 75 | 2253 | 120 | 2245 | 15 | 2247 | 45 |
| VH | 1809 | 345 | 1809 | 345 | 1809 | 345 | 1821 | 351 | 1821 | 351 |
| Tag | 2118 | 27 | 2118 | 27 | 2118 | 27 | 2118 | 27 | 2118 | 27 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2302 (TAU_ITR230) which comprises a 5′ inverted terminal repeat (ITR) sequence region and a 3′ ITR sequence region, a GFAP promoter, a human beta-globin intron region, a light chain signal sequence, a C10.2 antibody light chain variable region, a (G4S)3 linker (“(G4S)3” disclosed as SEQ ID NO: 4537), a C10.2 antibody heavy chain variable region, an HA tag, and a rabbit globin polyadenylation signal sequence.

In some embodiments, the AAV particle genome comprises SEQ ID NO: 2303 (TAU_ITR231) which comprises a 5′ inverted terminal repeat (ITR) sequence region and a 3′ ITR sequence region, a GFAP promoter, a human beta-globin intron region, a light chain signal sequence, a C10.2 antibody light chain variable region, a (G4S)5 linker (“(G4S)5” disclosed as SEQ ID NO: 4538), a C10.2 antibody heavy chain variable region, an HA tag, and a rabbit globin polyadenylation signal sequence.

In some embodiments, the AAV particle genome comprises SEQ ID NO: 2304 (TAU_ITR232) which comprises a 5′ inverted terminal repeat (ITR) sequence region and a 3′ ITR sequence region, a GFAP promoter, a human beta-globin intron region, a light chain signal sequence, a C10.2 antibody light chain variable region, a (G4S)8 linker (“G4S)8” disclosed as SEQ ID NO: 4542), a C10.2 antibody heavy chain variable region, an HA tag, and a rabbit globin polyadenylation signal sequence.

In some embodiments, the AAV particle genome comprises SEQ ID NO: 2276 (TAU_ITR204) which comprises a 5′ inverted terminal repeat (ITR) sequence region and a 3′ ITR sequence region, a GFAP promoter, a human beta-globin intron region, a light chain signal sequence, an IPN002 antibody light chain variable region, a G4S linker (“G4S” disclosed as SEQ ID NO: 4535), an IPN002 antibody heavy chain variable region, an HA tag, and a rabbit globin polyadenylation signal sequence.

In some embodiments, the AAV particle genome comprises SEQ ID NO: 2277 (TAU_ITR205) which comprises a 5′ inverted terminal repeat (ITR) sequence region and a 3′ ITR sequence region, a GFAP promoter, a human beta-globin intron region, a light chain signal sequence, an IPN002 antibody light chain variable region, a (G4S)3 linker (“(G4S)3” disclosed as SEQ ID NO: 4537), an IPN002 antibody heavy chain variable region, an HA tag, and a rabbit globin polyadenylation signal sequence.

TABLE 44
Sequence Regions in ITR to ITR Sequences

| | TAU_ITR94 | | TAU_ITR101 | | TAU_ ITR227 | | TAU_ITR95 | | TAU_ITR102 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2144) | | 2151) | | 2299) | | 2145) | | 2152) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2085 | 699 | 2085 | 699 | 2085 | 699 | 2086 | 557 | 2086 | 557 |
| Intron | 2240 | 566 | 2240 | 566 | 2240 | 566 | 2240 | 566 | 2240 | 566 |
| Signal | 1861 | 57 | 1861 | 57 | 1861 | 57 | 1861 | 57 | 1861 | 57 |
| VL | 1926 | 321 | 1926 | 321 | 1926 | 321 | 1926 | 321 | 1926 | 321 |
| Constant | 1864 | 321 | 1864 | 321 | 1864 | 321 | 1864 | 321 | 1864 | 321 |
| Linker | 1727 | 75 | 1726 | 54 | 1726 | 54 | 1727 | 75 | 1726 | 54 |
| Signal | 1740 | 57 | 1740 | 57 | 1740 | 57 | 1740 | 57 | 1740 | 57 |
| VH | 1809 | 345 | 1809 | 345 | 1809 | 345 | 1809 | 345 | 1809 | 345 |
| Constant | 1742 | 969 | 1743 | 972 | 2242 | 309 | 1742 | 969 | 1743 | 972 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2144 (TAU_ITR94) which comprises a 5′ inverted terminal repeat (ITR) sequence region and a 3′ ITR sequence region, a GFAP promoter, a human beta-globin intron region, a light chain signal sequence, a C10.2 antibody light chain variable region, a light chain constant region, a F2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2151 (TAU_ITR101) which comprises a 5′ inverted terminal repeat (ITR) sequence region and a 3′ ITR sequence region, a GFAP promoter, a human beta-globin intron region, a light chain signal sequence, a C10.2 antibody light chain variable region, a light chain constant region, a T2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2299 (TAU_ITR227) which comprises a 5′ inverted terminal repeat (ITR) sequence region and a 3′ ITR sequence region, a GFAP promoter, a human beta-globin intron region, a light chain signal sequence, a C10.2 antibody light chain variable region, a light chain constant region, a T2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2145 (TAU_ITR95) which comprises a 5′ inverted terminal repeat (ITR) sequence region and a 3′ ITR sequence region, a hSYN promoter, a human beta-globin intron region, a light chain signal sequence, a C10.2 antibody light chain variable region, a light chain constant region, a F2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2152 (TAU_ITR102) which comprises a 5′ inverted terminal repeat (ITR) sequence region and a 3′ ITR sequence region, a hSYN promoter, a human beta-globin intron region, a light chain signal sequence, a C10.2 antibody light chain variable region, a light chain constant region, a T2A linker, a heavy chain signal sequence, a C10.2 antibody heavy chain variable region, a heavy chain constant region, and a rabbit globin polyadenylation signal sequence.

TABLE 45
Sequence Regions in ITR to ITR Sequences

| | TAU_ITR229 | | TAU_ITR238 | | TAU_ITR239 | | TAU_ITR240 | | TAU_ITR237 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2301) | | 2310) | | 2311) | | 2312) | | 2309) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2085 | 699 | 2086 | 557 | 2086 | 557 | 2086 | 557 | 2086 | 557 |
| Intron | 2240 | 566 | 2240 | 566 | 2240 | 566 | 2240 | 566 | 2240 | 566 |
| Signal | 1861 | 57 | 1861 | 57 | 1861 | 57 | 1861 | 57 | 1861 | 57 |
| VL | 1926 | 321 | 1926 | 321 | 1926 | 321 | 1926 | 321 | 1926 | 321 |
| Linker | 2245 | 15 | 2247 | 45 | 2250 | 75 | 2253 | 120 | 2245 | 15 |
| VH | 1809 | 345 | 1809 | 345 | 1809 | 345 | 1809 | 345 | 1809 | 345 |
| Tag | 2118 | 27 | 2118 | 27 | 2118 | 27 | 2118 | 27 | 2118 | 27 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2301 (TAU_ITR229) which comprises a 5′ inverted terminal repeat (ITR) sequence region and a 3′ ITR sequence region, a GFAP promoter, a human beta-globin intron region, a light chain signal sequence, a C10.2 antibody light chain variable region, a G4S linker (“G4S” disclosed as SEQ ID NO: 4535), a C10.2 antibody heavy chain variable region, an HA tag, and a rabbit globin polyadenylation signal sequence.

In some embodiments, the AAV particle genome comprises SEQ ID NO: 2310 (TAU_ITR238) which comprises a 5′ inverted terminal repeat (ITR) sequence region and a 3′ ITR sequence region, a hSYN promoter, a human beta-globin intron region, a light chain signal sequence, a C10.2 antibody light chain variable region, a (G4S)3 linker (“(G4S)3” disclosed as SEQ ID NO: 4537), a C10.2 antibody heavy chain variable region, an HA tag, and a rabbit globin polyadenylation signal sequence.

In some embodiments, the AAV particle genome comprises SEQ ID NO: 2311 (TAU_ITR239) which comprises a 5′ inverted terminal repeat (ITR) sequence region and a 3′ ITR sequence region, a hSYN promoter, a human beta-globin intron region, a light chain signal sequence, a C10.2 antibody light chain variable region, a (G4S)5 linker (“(G4S)5” disclosed as SEQ ID NO: 4538), a C10.2 antibody heavy chain variable region, an HA tag, and a rabbit globin polyadenylation signal sequence.

In some embodiments, the AAV particle genome comprises SEQ ID NO: 2312 (TAU_ITR240) which comprises a 5′ inverted terminal repeat (ITR) sequence region and a 3′ ITR sequence region, a hSYN promoter, a human beta-globin intron region, a light chain signal sequence, a C10.2 antibody light chain variable region, a (G4S)8 linker (“(G4S)8” disclosed as SEQ ID NO: 4542), a C10.2 antibody heavy chain variable region, an HA tag, and a rabbit globin polyadenylation signal sequence.

In some embodiments, the AAV particle genome comprises SEQ ID NO: 2309 (TAU_ITR237) which comprises a 5′ inverted terminal repeat (ITR) sequence region and a 3′ ITR sequence region, a hSYN promoter, a human beta-globin intron region, a light chain signal sequence, a C10.2 antibody light chain variable region, a G4S linker (“G4S” disclosed as SEQ ID NO: 4535), a C10.2 antibody heavy chain variable region, an HA tag, and a rabbit globin polyadenylation signal sequence.

TABLE 46
Sequence Regions in ITR to ITR Sequences

| | TAU_ITR91 | | TAU_ITR99 | | TAU_ITR234 | | TAU_ITR236 | | TAU_ITR208 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2141) | | 2149) | | 2306) | | 2308) | | 2280) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2086 | 557 | 2086 | 557 | 2086 | 557 | 2086 | 557 | 2086 | 557 |
| Intron | 2240 | 566 | 2240 | 566 | 2240 | 566 | 2240 | 566 | 2240 | 566 |
| Signal | 1740 | 57 | 1740 | 57 | 1740 | 57 | 1740 | 57 | 1740 | 57 |
| VH | 1809 | 345 | 1809 | 345 | 1809 | 345 | 1809 | 345 | 1821 | 351 |
| Constant | 1742 | 969 | 1742 | 969 | 2242 | 309 | 2243 | 291 | 2242 | 309 |
| Linker | 1725 | 12 | 1724 | 12 | 1724 | 12 | 1724 | 12 | 1724 | 12 |
| Linker | 1727 | 75 | 1726 | 54 | 1726 | 54 | 1726 | 54 | 1726 | 54 |
| Signal | 1861 | 57 | 1861 | 57 | 1861 | 57 | 1861 | 57 | 1861 | 57 |
| VL | 1926 | 321 | 1926 | 321 | 1926 | 321 | 1926 | 321 | 1939 | 336 |
| Constant | 1864 | 321 | 1864 | 321 | 1864 | 321 | 1864 | 321 | 1864 | 321 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2141 (TAU_ITR91) which comprises a 5′ inverted terminal repeat (ITR) sequence region and a 3′ ITR sequence region, a hSYN promoter, a human beta-globin intron region, a heavy chain signal sequence, a C10.2 antibody heavy chain variable region, a heavy chain constant region, a furin cleavage site, a F2A linker, a light chain signal sequence, a C10.2 antibody light chain variable region, a light chain constant region, and a rabbit globin polyadenylation signal sequence.

In some embodiments, the AAV particle genome comprises SEQ ID NO: 2149 (TAU_ITR99) which comprises a 5′ inverted terminal repeat (ITR) sequence region and a 3′ ITR sequence region, a hSYN promoter, a human beta-globin intron region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2306 (TAU_ITR234) which comprises a 5′ inverted terminal repeat (ITR) sequence region and a 3′ ITR sequence region, a hSYN promoter, a human beta-globin intron region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2308 (TAU_ITR236) which comprises a 5′ inverted terminal repeat (ITR) sequence region and a 3′ ITR sequence region, a hSYN promoter, a human beta-globin intron region, a heavy chain signal sequence, a C10.2 antibody heavy chain variable region, a heavy chain constant region, a furin cleavage site, a T2A linker, a light chain signal sequence, a C10.2 antibody light chain variable region, a light chain constant region, and a rabbit globin polyadenylation signal sequence.

In some embodiments, the AAV particle genome comprises SEQ ID NO: 2280 (TAU_ITR208) which comprises a 5′ inverted terminal repeat (ITR) sequence region and a 3′ ITR sequence region, a hSYN promoter, a human beta-globin intron region, a heavy chain signal sequence, an IPN002 antibody heavy chain variable region, a heavy chain constant region, a furin cleavage site, a T2A linker, a light chain signal sequence, an IPN002 antibody light chain variable region, a light chain constant region, and a rabbit globin polyadenylation signal sequence.

TABLE 47
Sequence Regions in ITR to ITR Sequences

| | |
|---|---|
| Sequence | TAU_ITR233 (SEQ ID NO: 2305) |

| Regions | Region SEQ ID NO | Region length |
|---|---|---|
| 5′ ITR | 2076 | 130 |
| Promoter | 2086 | 557 |
| Intron | 2240 | 566 |
| Signal | 1740 | 57 |
| VH | 1809 | 345 |
| Constant | 2242 | 309 |
| Linker | 2244 | 198 |
| Linker | 1724 | 12 |
| Linker | 1726 | 54 |
| Signal | 1861 | 57 |
| VL | 1926 | 321 |
| Constant | 1864 | 321 |
| PolyA | 2122 | 127 |
| 3′ ITR | 2078 | 130 |

In some embodiments, the AAV particle genome comprises SEQ ID NO: 2305 (TAU_ITR233) which comprises a 5′ inverted terminal repeat (ITR) sequence region and a 3′ ITR sequence region, a hSYN promoter, a human beta-globin intron region, a heavy chain signal sequence, a C10.2 antibody heavy chain variable region, a heavy chain constant region, a hinge region, a furin cleavage site, a T2A linker, a light chain signal sequence, a C10.2 antibody light chain variable region, a light chain constant region, and a rabbit globin polyadenylation signal sequence.

TABLE 48
Sequence Regions in ITR to ITR Sequences

| | TAU_ITR235 | | TAU_ITR199 | | TAU_ITR209 | | TAU_ITR129 | |
|---|---|---|---|---|---|---|---|---|
| | (SEQ ID NO: | | (SEQ ID NO: | | (SEQ ID NO: | | (SEQ ID NO: | |
| | 2307) | | 2271) | | 2281) | | 2179) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2086 | 557 | 2085 | 699 | 2086 | 557 | 2239 | 380 |
| Intron | 2240 | 566 | 2240 | 566 | 2240 | 566 | 2240 | 566 |
| Signal | 1861 | 57 | 1861 | 57 | 1861 | 57 | 1861 | 57 |
| VL | 1926 | 321 | 1939 | 336 | 1939 | 336 | 1939 | 336 |
| Constant | 1864 | 321 | 1864 | 321 | 1864 | 321 | 1864 | 321 |
| Linker | 1726 | 54 | 1726 | 54 | 1726 | 54 | 1726 | 54 |
| Signal | 1740 | 57 | 1740 | 57 | 1740 | 57 | 1740 | 57 |
| VH | 1809 | 345 | 1821 | 351 | 1821 | 351 | 1821 | 351 |
| Constant | 2242 | 309 | 2242 | 309 | 2242 | 309 | 2242 | 309 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2307 (TAU_ITR235) which comprises a 5′ inverted terminal repeat (ITR) sequence region and a 3′ ITR sequence region, a hSYN promoter, a human beta-globin intron region, a light chain signal sequence, a C10.2 antibody light chain variable region, a light chain constant region, a T2A linker, a heavy chain signal sequence, a C10.2 antibody heavy chain variable region, a heavy chain constant region, and a rabbit globin polyadenylation signal sequence.

In some embodiments, the AAV particle genome comprises SEQ ID NO: 2271 (TAU_ITR199) which comprises a 5′ inverted terminal repeat (ITR) sequence region and a 3′ ITR sequence region, a GFAP promoter, a human beta-globin intron region, a light chain signal sequence, an IPN002 antibody light chain variable region, a light chain constant region, a T2A linker, a heavy chain signal sequence, an IPN002 antibody heavy chain variable region, a heavy chain constant region, and a rabbit globin polyadenylation signal sequence.

In some embodiments, the AAV particle genome comprises SEQ ID NO: 2281 (TAU_ITR209) which comprises a 5′ inverted terminal repeat (ITR) sequence region and a 3′ ITR sequence region, a hSYN promoter, a human beta-globin intron region, a light chain signal sequence, an IPN002 antibody light chain variable region, a light chain constant region, a T2A linker, a heavy chain signal sequence, an IPN002 antibody heavy chain variable region, a heavy chain constant region, and a rabbit globin polyadenylation signal sequence.

In some embodiments, the AAV particle genome comprises SEQ ID NO: 2179 (TAU_ITR129) which comprises a 5′ inverted terminal repeat (ITR) sequence region and a 3′ ITR sequence region, a CMVie region, a human beta-globin intron region, a light chain signal sequence, an IPN002 antibody light chain variable region, a light chain constant region, a T2A linker, a heavy chain signal sequence, an IPN002 antibody heavy chain variable region, a heavy chain constant region, and a rabbit globin polyadenylation signal sequence.

TABLE 49
Sequence Regions in ITR to ITR Sequences

| | |
|---|---|
| TAU_ITR88 | TAU_ITR96 |
| (SEQ ID NO: 2138) | (SEQ ID NO: 2146) |

| Sequence | Region SEQ | Region | Region SEQ | Region |
|---|---|---|---|---|
| Regions | ID NO | length | ID NO | length |
| 5′ ITR | 2076 | 130 | 2076 | 130 |
| Promoter | 2239 | 380 | 2239 | 380 |
| Promoter | 2083 | 260 | 2083 | 260 |
| Intron | 2240 | 566 | 2240 | 566 |
| Signal | 1740 | 57 | 1740 | 57 |
| VH | 1809 | 345 | 1809 | 345 |
| Constant | 1742 | 969 | 1742 | 969 |
| Linker | 1725 | 12 | 1724 | 12 |
| Linker | 1727 | 75 | 1726 | 54 |
| Signal | 1861 | 57 | 1861 | 57 |
| VL | 1926 | 321 | 1926 | 321 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 2138 (TAU_ITR88) which comprises a 5′ inverted terminal repeat (ITR) sequence region and a 3′ ITR sequence region, a CMVie region, a CB promoter, a human beta-globin intron region, a heavy chain signal sequence, a C10.2 antibody heavy chain variable region, a heavy chain constant region, a furin cleavage site, an F2A linker, a light chain signal sequence, a C10.2 antibody light chain variable region, a light chain constant region, and a rabbit globin polyadenylation signal sequence.

In some embodiments, the AAV particle genome comprises SEQ ID NO: 2146 (TAU_ITR96) which comprises a 5′ inverted terminal repeat (ITR) sequence region and a 3′ ITR sequence region, a CMVie region, a CB promoter, a human beta-globin intron region, a heavy chain signal sequence, a C10.2 antibody heavy chain variable region, a heavy chain constant region, a furin cleavage site, an T2A linker, a light chain signal sequence, a C10.2 antibody light chain variable region, a light chain constant region, and a rabbit globin polyadenylation signal sequence.

TABLE 50
Sequence Regions in ITR to ITR Sequences

| | |
|---|---|
| Sequence | TAU_ITR92 (SEQ ID NO: 2142) |

| Regions | Region SEQ ID NO | Region length |
|---|---|---|
| 5′ ITR | 2076 | 130 |
| Promoter | 2239 | 380 |
| Promoter | 2083 | 260 |
| Intron | 2240 | 566 |
| Signal | 1861 | 57 |
| VL | 1926 | 321 |
| Constant | 1864 | 321 |
| Linker | 1727 | 75 |
| Signal | 1740 | 57 |
| VH | 1809 | 345 |
| Constant | 1742 | 969 |
| PolyA | 2122 | 127 |
| 3′ ITR | 2078 | 130 |

In some embodiments, the AAV particle genome comprises SEQ ID NO: 2142 (TAU_ITR92) which comprises a 5′ inverted terminal repeat (ITR) sequence region and a 3′ ITR sequence region, a CMVie region, a CB promoter, a human beta-globin intron region, a light chain signal sequence, a C10.2 antibody light chain variable region, a light chain constant region, an F2A linker, a heavy chain signal sequence, a C10.2 antibody heavy chain variable region, a heavy chain constant region, and a rabbit globin polyadenylation signal sequence.

TABLE 51
Sequence Regions in ITR to ITR Sequences

| | TAU_ITR139 | | TAU_ITR140 | | TAU_ITR141 | | TAU_ITR142 | | TAU_ITR143 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2189) | | 2190) | | 2191) | | 2192) | | 2193) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| CAG | 2080 | 1715 | 2080 | 1715 | 2080 | 1715 | 2080 | 1715 | 2080 | 1715 |
| promoter | | | | | | | | | | |
| CMVie | 2081 | 299 | 2081 | 299 | 2081 | 299 | 2081 | 299 | 2081 | 299 |
| CBA min. | 2082 | 283 | 2082 | 283 | 2082 | 283 | 2082 | 283 | 2082 | 283 |
| Signal | 1740 | 57 | 1740 | 57 | 1740 | 57 | 1740 | 57 | 1740 | 57 |
| VH | 1821 | 351 | 1821 | 351 | 1821 | 351 | 1821 | 351 | 1821 | 351 |
| Linker | 1730 | 45 | 2254 | 60 | 2249 | 75 | 2251 | 90 | 2252 | 120 |
| VL | 1939 | 336 | 1939 | 336 | 1939 | 336 | 1939 | 336 | 1939 | 336 |
| Tag | 2118 | 27 | 2118 | 27 | 2118 | 27 | 2118 | 27 | 2118 | 27 |
| PolyA | 2122 | 127 | 2122 | 127 | 2122 | 127 | 2122 | 127 | 2122 | 127 |
| 3′ ITR | 2078 | 130 | 2078 | 130 | 2078 | 136 | 2078 | 130 | 2078 | 130 |

In some embodiments, the AAV particle genome comprises SEQ ID NO: 2189 (TAU_ITR139) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a (G4S)3 linker (“(G4S)3” disclosed as SEQ ID NO: 4537), an IPN002 antibody light chain variable region, an HA tag, and a rabbit globin polyadenylation signal sequence.

In some embodiments, the AAV particle genome comprises SEQ ID NO: 2190 (TAU_ITR140) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a (G4S)4 linker (“(G4S)4” disclosed as SEQ ID NO: 4540), an IPN002 antibody light chain variable region, an HA tag, and a rabbit globin polyadenylation signal sequence.

In some embodiments, the AAV particle genome comprises SEQ ID NO: 2191 (TAU_ITR141) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a (G4S)5 linker (“(G4S)5” disclosed as SEQ ID NO: 4538), an IPN002 antibody light chain variable region, an HA tag, and a rabbit globin polyadenylation signal sequence.

In some embodiments, the AAV particle genome comprises SEQ ID NO: 2192 (TAU_ITR142) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a (G4S)6 linker (“(G4S)4” disclosed as SEQ ID NO: 4541), an IPN002 antibody light chain variable region, an HA tag, and a rabbit globin polyadenylation signal sequence.

In some embodiments, the AAV particle genome comprises SEQ ID NO: 2193 (TAU_ITR143) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a (G4S)8 linker (“(G4S)8” disclosed as SEQ ID NO: 4542), an IPN002 antibody light chain variable region, an HA tag, and a rabbit globin polyadenylation signal sequence.

TABLE 52
Sequence Regions in ITR to ITR Sequences

| | |
|---|---|
| Sequence | TAU_ITR137 (SEQ ID NO: 2187) |

| Regions | Region SEQ ID NO | Region length |
|---|---|---|
| 5′ ITR | 2076 | 130 |
| CAG promoter | 2080 | 1715 |
| CMVie | 2081 | 299 |
| CBA min. | 2082 | 283 |
| Signal | 1740 | 57 |
| VH | 1821 | 351 |
| Linker | 2245 | 15 |
| VL | 1939 | 336 |
| Tag | 2118 | 27 |
| PolyA | 2122 | 127 |
| 3′ ITR | 2078 | 130 |

In some embodiments, the AAV particle genome comprises SEQ ID NO: 2187 (TAU_ITR137) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a G4S linker (“G4S” disclosed as SEQ ID NO: 4535), an IPN002 antibody light chain variable region, an HA tag, and a rabbit globin polyadenylation signal sequence.

TABLE 53
Sequence Regions in ITR to ITR Sequences

| | TAU_ITR125 | | TAU_ITR127 | | TAU_ITR128 | |
|---|---|---|---|---|---|---|
| | (SEQ ID NO: | | (SEQ ID NO: | | (SEQ ID NO: | |
| | 2175) | | 2177) | | 2178) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| CAG | 2080 | 1715 | 2080 | 1715 | 2080 | 1715 |
| promoter | | | | | | |
| CMVie | 2081 | 299 | 2081 | 299 | 2081 | 299 |
| CBA min. | 2082 | 283 | 2082 | 283 | 2082 | 283 |
| Signal | 1740 | 57 | 1740 | 57 | 1740 | 57 |
| VH | 1821 | 351 | 1821 | 351 | 1821 | 351 |
| Constant | 2242 | 309 | 2243 | 291 | 2242 | 309 |
| Linker | 1724 | 12 | 1724 | 12 | 1724 | 12 |
| Linker | 1726 | 54 | 1726 | 54 | 1726 | 54 |
| Signal | 1861 | 57 | 1861 | 57 | 1861 | 57 |
| VL | 1939 | 336 | 1939 | 336 | 1939 | 336 |
| Constant | 1864 | 321 | 1864 | 321 | 1864 | 321 |
| PolyA | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2175 (TAU_ITR125) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a heavy chain constant region, a furin cleavage site, a T2A linker, a light chain signal sequence, an IPN002 antibody light chain variable region, a light chain constant region, and a rabbit globin polyadenylation signal sequence.

In some embodiments, the AAV particle genome comprises SEQ ID NO: 2177 (TAU_ITR127) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a heavy chain constant region, a furin cleavage site, a T2A linker, a light chain signal sequence, an IPN002 antibody light chain variable region, a light chain constant region, and a rabbit globin polyadenylation signal sequence.

In some embodiments, the AAV particle genome comprises SEQ ID NO: 2178 (TAU_ITR128) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a heavy chain constant region, a furin cleavage site, a T2A linker, a light chain signal sequence, an IPN002 antibody light chain variable region, a light chain constant region, and a rabbit globin polyadenylation signal sequence.

TABLE 54
Sequence Regions in ITR to ITR Sequences

| | |
|---|---|
| Sequence | TAU_ITR126 (SEQ ID NO: 2176) |

| Regions | Region SEQ ID NO | Region length |
|---|---|---|
| 5′ ITR | 2076 | 130 |
| CAG promoter | 2080 | 1715 |
| CMVie | 2081 | 299 |
| CBA min. | 2082 | 283 |
| Signal | 1740 | 57 |
| VH | 1821 | 351 |
| Constant | 2242 | 309 |
| Linker | 2244 | 198 |
| Linker | 1724 | 12 |
| Linker | 1726 | 54 |
| Signal | 1861 | 57 |
| VL | 1939 | 336 |
| Constant | 1864 | 321 |
| PolyA | 2122 | 127 |
| 3′ ITR | 2078 | 130 |

In some embodiments, the AAV particle genome comprises SEQ ID NO: 2176 (TAU_ITR126) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, a heavy chain constant region, a hinge region, a furin cleavage site, a T2A linker, a light chain signal sequence, an IPN002 antibody light chain variable region, a light chain constant region, and a rabbit globin polyadenylation signal sequence.

TABLE 55
Sequence Regions in ITR to ITR Sequences

| | |
|---|---|
| Sequence | TAU_ITR138 (SEQ ID NO: 2188) |

| Regions | Region SEQ ID NO | Region length |
|---|---|---|
| 5′ ITR | 2076 | 130 |
| CAG promoter | 2080 | 1715 |
| CMVie | 2081 | 299 |
| CBA min. | 2082 | 283 |
| Signal | 1740 | 57 |
| VH | 1821 | 351 |
| VL | 1939 | 336 |
| Tag | 2118 | 27 |
| PolyA | 2122 | 127 |
| 3′ ITR | 2078 | 130 |

In some embodiments, the AAV particle genome comprises SEQ ID NO: 2188 (TAU_ITR138) which comprises a 5′ inverted terminal repeat (ITR) sequence region and a 3′ ITR sequence region, a CAG promoter comprising a CMVie region and a minimal CBA promoter region, a heavy chain signal sequence, an IPN002 antibody heavy chain variable region, an IPN002 antibody light chain variable region, an HA tag, and a rabbit globin polyadenylation signal sequence.

TABLE 56
Sequence Regions in ITR to ITR Sequences

| | TAU_ITR152 | | TAU_ITR153 | | TAU_ITR154 | | TAU_ITRI55 | | TAU_ITR150 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2202) | | 2203) | | 2204) | | 2205) | | 2200) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| CAG | 2238 | 1714 | 2238 | 1714 | 2238 | 1714 | 2238 | 1714 | 2080 | 1715 |
| promoter | | | | | | | | | | |
| CMVie | 2081 | 299 | 2081 | 299 | 2081 | 299 | 2081 | 299 | 2081 | 299 |
| CBA min. | 2082 | 283 | 2082 | 283 | 2082 | 283 | 2082 | 283 | 2082 | 283 |
| Signal | 1861 | 57 | 1861 | 57 | 1861 | 57 | 1861 | 57 | 1861 | 57 |
| VL | 1939 | 336 | 1939 | 336 | 1939 | 336 | 1939 | 336 | 1939 | 336 |
| Linker | 2247 | 45 | 2248 | 60 | 2250 | 75 | 2259 | 90 | 2245 | 15 |
| VH | 1821 | 351 | 1821 | 351 | 1821 | 351 | 1821 | 351 | 1821 | 351 |
| Tag | 2118 | 27 | 2118 | 27 | 2118 | 27 | 2118 | 27 | 2118 | 27 |
| PolyA | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2202 (TAU_ITR152) which comprises a 5′ inverted terminal repeat (ITR) sequence region and a 3′ ITR sequence region, a CAG promoter comprising a CMVie region and a minimal CBA promoter region, a light chain signal sequence, an IPN002 antibody light chain variable region, a (G4S)3 linker (“(G4S)3” disclosed as SEQ ID NO: 4537), an IPN002 antibody heavy chain variable region, an HA tag, and a rabbit globin polyadenylation signal sequence.

In some embodiments, the AAV particle genome comprises SEQ ID NO: 2203 (TAU_ITR153) which comprises a 5′ inverted terminal repeat (ITR) sequence region and a 3′ ITR sequence region, a CAG promoter comprising a CMVie region and a minimal CBA promoter region, a light chain signal sequence, an IPN002 antibody light chain variable region, a (G4S)4 linker (“(G4S)4” disclosed as SEQ ID NO: 4540), an IPN002 antibody heavy chain variable region, an HA tag, and a rabbit globin polyadenylation signal sequence.

In some embodiments, the AAV particle genome comprises SEQ ID NO: 2204 (TAU_ITR154) which comprises a 5′ inverted terminal repeat (ITR) sequence region and a 3′ ITR sequence region, a CAG promoter comprising a CMVie region and a minimal CBA promoter region, a light chain signal sequence, an IPN002 antibody light chain variable region, a (G4S)5 linker (“(G4S)5” disclosed as SEQ ID NO: 4538), an IPN002 antibody heavy chain variable region, an HA tag, and a rabbit globin polyadenylation signal sequence.

In some embodiments, the AAV particle genome comprises SEQ ID NO: 2205 (TAU_ITR155) which comprises a 5′ inverted terminal repeat (ITR) sequence region and a 3′ ITR sequence region, a CAG promoter comprising a CMVie region and a minimal CBA promoter region, a light chain signal sequence, an IPN002 antibody light chain variable region, a (G4S)6 linker (“(G4S)4” disclosed as SEQ ID NO: 4541), an IPN002 antibody heavy chain variable region, an HA tag, and a rabbit globin polyadenylation signal sequence.

In some embodiments, the AAV particle genome comprises SEQ ID NO: 2200 (TAU_ITR150) which comprises a 5′ inverted terminal repeat (ITR) sequence region and a 3′ ITR sequence region, a CAG promoter comprising a CMVie region and a minimal CBA promoter region, a light chain signal sequence, an IPN002 antibody light chain variable region, a G4S linker (“G4S” disclosed as SEQ ID NO: 4535), an IPN002 antibody heavy chain variable region, an HA tag, and a rabbit globin polyadenylation signal sequence.

TABLE 57
Sequence Regions in ITR to ITR Sequences

| | TAU_ITR200 | | TAU_ITR201 | | TAU_ITR202 | | TAU_ITR203 | | TAU_ITR211 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2272) | | 2273) | | 2274) | | 2275) | | 2283) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2085 | 699 | 2085 | 699 | 2085 | 699 | 2085 | 699 | 2086 | 557 |
| Intron | 2240 | 566 | 2240 | 566 | 2240 | 566 | 2240 | 566 | 2240 | 566 |
| Signal | 1740 | 57 | 1740 | 57 | 1740 | 57 | 1740 | 57 | 1740 | 57 |
| VH | 1821 | 351 | 1821 | 351 | 1821 | 351 | 1821 | 351 | 1821 | 351 |
| Linker | 2245 | 15 | 1730 | 45 | 2249 | 75 | 2252 | 120 | 1730 | 45 |
| VL | 1939 | 336 | 1939 | 336 | 1939 | 336 | 1939 | 336 | 1939 | 336 |
| Tag | 2118 | 27 | 2118 | 27 | 2118 | 27 | 2118 | 27 | 2118 | 27 |
| PolyA | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2272 (TAU_ITR200) which comprises a 5′ inverted terminal repeat (ITR) sequence region and a 3′ ITR sequence region, a GFAP promoter, a human beta-globin intron region, a heavy chain signal sequence, an IPN002 antibody heavy chain variable region, a G4S linker (“G4S” disclosed as SEQ ID NO: 4535), an IPN002 antibody light chain variable region, an HA tag, and a rabbit globin polyadenylation signal sequence.

In some embodiments, the AAV particle genome comprises SEQ ID NO: 2273 (TAU_ITR201) which comprises a 5′ inverted terminal repeat (ITR) sequence region and a 3′ ITR sequence region, a GFAP promoter, a human beta-globin intron region, a heavy chain signal sequence, an IPN002 antibody heavy chain variable region, a (G4S)3 linker (“(G4S)3” disclosed as SEQ ID NO: 4537), an IPN002 antibody light chain variable region, an HA tag, and a rabbit globin polyadenylation signal sequence.

In some embodiments, the AAV particle genome comprises SEQ ID NO: 2274 (TAU_ITR202) which comprises a 5′ inverted terminal repeat (ITR) sequence region and a 3′ ITR sequence region, a GFAP promoter, a human beta-globin intron region, a heavy chain signal sequence, an IPN002 antibody heavy chain variable region, a (G4S)5 linker (“(G4S)5” disclosed as SEQ ID NO: 4538), an IPN002 antibody light chain variable region, an HA tag, and a rabbit globin polyadenylation signal sequence.

In some embodiments, the AAV particle genome comprises SEQ ID NO: 2275 (TAU_ITR203) which comprises a 5′ inverted terminal repeat (ITR) sequence region and a 3′ ITR sequence region, a GFAP promoter, a human beta-globin intron region, a heavy chain signal sequence, an IPN002 antibody heavy chain variable region, a (G4S)8 linker (“(G4S)8” disclosed as SEQ ID NO: 4542), an IPN002 antibody light chain variable region, an HA tag, and a rabbit globin polyadenylation signal sequence.

In some embodiments, the AAV particle genome comprises SEQ ID NO: 2283 (TAU_ITR211) which comprises a 5′ inverted terminal repeat (ITR) sequence region and a 3′ ITR sequence region, a hSYN promoter, a human beta-globin intron region, a heavy chain signal sequence, an IPN002 antibody heavy chain variable region, a (G4S)3 linker (“(G4S)3” disclosed as SEQ ID NO: 4537), an IPN002 antibody light chain variable region, an HA tag, and a rabbit globin polyadenylation signal sequence.

TABLE 58
Sequence Regions in ITR to ITR Sequences

| | TAU_ITR206 | | TAU_ITR215 | | TAU_ITR216 | | TAU_ITR214 | | TAU_ITR144 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2278) | | 2287) | | 2288) | | 2286) | | 2194) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2085 | 699 | 2086 | 557 | 2086 | 557 | 2086 | 557 | 2239 | 380 |
| Intron | 2240 | 566 | 2240 | 566 | 2240 | 566 | 2240 | 566 | 2240 | 566 |
| Signal | 1861 | 57 | 1861 | 57 | 1861 | 57 | 1861 | 57 | 1861 | 57 |
| VL | 1939 | 336 | 1939 | 336 | 1939 | 336 | 1939 | 336 | 1939 | 336 |
| Linker | 2250 | 75 | 2247 | 45 | 2250 | 75 | 2245 | 15 | 2245 | 15 |
| VH | 1821 | 351 | 1821 | 351 | 1821 | 351 | 1821 | 351 | 1821 | 351 |
| Tag | 2118 | 27 | 2118 | 27 | 2118 | 27 | 2118 | 27 | 2118 | 27 |
| PolyA | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2278 (TAU_ITR206) which comprises a 5′ inverted terminal repeat (ITR) sequence region and a 3′ ITR sequence region, a GFAP promoter, a human beta-globin intron region, a light chain signal sequence, an IPN002 antibody light chain variable region, a (G4S)5 linker (“(G4S)5” disclosed as SEQ ID NO: 4538), an IPN002 antibody heavy chain variable region, an HA tag, and a rabbit globin polyadenylation signal sequence.

In some embodiments, the AAV particle genome comprises SEQ ID NO: 2287 (TAU_ITR215) which comprises a 5′ inverted terminal repeat (ITR) sequence region and a 3′ ITR sequence region, a hSYN promoter, a human beta-globin intron region, a light chain signal sequence, an IPN002 antibody light chain variable region, a (G4S)3 linker (“(G4S)3” disclosed as SEQ ID NO: 4537), an IPN002 antibody heavy chain variable region, an HA tag, and a rabbit globin polyadenylation signal sequence.

In some embodiments, the AAV particle genome comprises SEQ ID NO: 2288 (TAU_ITR216) which comprises a 5′ inverted terminal repeat (ITR) sequence region and a 3′ ITR sequence region, a hSYN promoter, a human beta-globin intron region, a light chain signal sequence, an IPN002 antibody light chain variable region, a (G4S)5 linker (“(G4S)5” disclosed as SEQ ID NO: 4538), an IPN002 antibody heavy chain variable region, an HA tag, and a rabbit globin polyadenylation signal sequence.

In some embodiments, the AAV particle genome comprises SEQ ID NO: 2286 (TAU_ITR214) which comprises a 5′ inverted terminal repeat (ITR) sequence region and a 3′ ITR sequence region, a hSYN promoter, a human beta-globin intron region, a light chain signal sequence, an IPN002 antibody light chain variable region, a G4S linker (“G4S” disclosed as SEQ ID NO: 4535), an IPN002 antibody heavy chain variable region, an HA tag, and a rabbit globin polyadenylation signal sequence.

In some embodiments, the AAV particle genome comprises SEQ ID NO: 2194 (TAU_ITR144) which comprises a 5′ inverted terminal repeat (ITR) sequence region and a 3′ ITR sequence region, a CMVie region, a human beta-globin intron region, a light chain signal sequence, an IPN002 antibody light chain variable region, a G4S linker (“G4S” disclosed as SEQ ID NO: 4535), an IPN002 antibody heavy chain variable region, an HA tag, and a rabbit globin polyadenylation signal sequence.

TABLE 59
Sequence Regions in ITR to ITR Sequences

| | TAU_ITR212 | | TAU_ITR213 | | TAU_ITR210 | |
|---|---|---|---|---|---|---|
| | (SEQ ID NO: | | (SEQ ID NO: | | (SEQ ID NO: | |
| | 2284) | | 2285) | | 2282) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| Promoter | 2086 | 557 | 2086 | 557 | 2086 | 557 |
| Intron | 2240 | 566 | 2240 | 566 | 2240 | 566 |
| Signal | 1740 | 57 | 1740 | 57 | 1740 | 57 |
| VH | 1821 | 351 | 1821 | 351 | 1821 | 351 |
| Linker | 2249 | 75 | 2252 | 120 | 2245 | 15 |
| VL | 1939 | 336 | 1939 | 336 | 1939 | 336 |
| Tag | 2118 | 27 | 2118 | 27 | 2118 | 27 |
| PolyA | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2284 (TAU_ITR212) which comprises a 5′ inverted terminal repeat (ITR) sequence region and a 3′ ITR sequence region, a hSYN promoter, a human beta-globin intron region, a heavy chain signal sequence, an IPN002 antibody heavy chain variable region, a (G4S)5 linker (“(G4S)5” disclosed as SEQ ID NO: 4538), an IPN002 antibody light chain variable region, an HA tag, and a rabbit globin polyadenylation signal sequence.

In some embodiments, the AAV particle genome comprises SEQ ID NO: 2285 (TAU_ITR213) which comprises a 5′ inverted terminal repeat (ITR) sequence region and a 3′ ITR sequence region, a hSYN promoter, a human beta-globin intron region, a heavy chain signal sequence, an IPN002 antibody heavy chain variable region, a (G4S)8 linker (“(G4S)8” disclosed as SEQ ID NO: 4542), an IPN002 antibody light chain variable region, an HA tag, and a rabbit globin polyadenylation signal sequence.

In some embodiments, the AAV particle genome comprises SEQ ID NO: 2282 (TAU_ITR210) which comprises a 5′ inverted terminal repeat (ITR) sequence region and a 3′ ITR sequence region, a hSYN promoter, a human beta-globin intron region, a heavy chain signal sequence, an IPN002 antibody heavy chain variable region, a G4S linker (G4S″ disclosed as SEQ ID NO: 4535), an IPN002 antibody light chain variable region, an HA tag, and a rabbit globin polyadenylation signal sequence.

TABLE 60
Sequence Regions in ITR to ITR Sequences

| | TAU_ITR131 | | TAU_ITR132 | | TAU_ITR133 | | TAU_ITR134 | | TAU_ITR135 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2181) | | 2182) | | 2183) | | 2184) | | 2185) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2239 | 380 | 2239 | 380 | 2239 | 380 | 2239 | 380 | 2239 | 380 |
| Intron | 2240 | 566 | 2240 | 566 | 2240 | 566 | 2240 | 566 | 2240 | 566 |
| Signal | 1740 | 57 | 1740 | 57 | 1740 | 57 | 1740 | 57 | 1740 | 57 |
| VH | 1821 | 351 | 1821 | 351 | 1821 | 351 | 1821 | 351 | 1821 | 351 |
| Constant | 1864 | 321 | 1864 | 321 | 1864 | 321 | 1864 | 321 | 1864 | 321 |
| Linker | 2245 | 15 | 2246 | 30 | 1730 | 45 | 2254 | 60 | 2249 | 75 |
| VL | 1939 | 336 | 1939 | 336 | 1939 | 336 | 1939 | 336 | 1939 | 336 |
| Tag | 2118 | 27 | 2118 | 27 | 2118 | 27 | 2118 | 27 | 2118 | 27 |
| PolyA | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2181 (TAU_ITR131) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a light chain constant region, a G4S linker (“G4S” disclosed as SEQ ID NO: 4535), an IPN002 antibody light chain variable region, an HA tag, and a rabbit globin polyadenylation signal sequence.

In some embodiments, the AAV particle genome comprises SEQ ID NO: 2182 (TAU_ITR132) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a light chain constant region, a (G4S)2 linker (“(G4S)4” disclosed as SEQ ID NO: 4539), an IPN002 antibody light chain variable region, an HA tag, and a rabbit globin polyadenylation signal sequence.

In some embodiments, the AAV particle genome comprises SEQ ID NO: 2183 (TAU_ITR133) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a light chain constant region, a (G4S)3 linker (“(G4S)3” disclosed as SEQ ID NO: 4537), an IPN002 antibody light chain variable region, an HA tag, and a rabbit globin polyadenylation signal sequence.

In some embodiments, the AAV particle genome comprises SEQ ID NO: 2184 (TAU_ITR134) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a light chain constant region, a (G4S)4 linker (“(G4S)4” disclosed as SEQ ID NO: 4540), an IPN002 antibody light chain variable region, an HA tag, and a rabbit globin polyadenylation signal sequence.

In some embodiments, the AAV particle genome comprises SEQ ID NO: 2185 (TAU_ITR135) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a light chain constant region, a (G4S)5 linker (“(G4S)5” disclosed as SEQ ID NO: 4538), an IPN002 antibody light chain variable region, an HA tag, and a rabbit globin polyadenylation signal sequence.

TABLE 61
Sequence Regions in ITR to ITR Sequences

| | |
|---|---|
| Sequence | TAU_ITR136 (SEQ ID NO: 2186) |

| Regions | Region SEQ ID NO | Region length |
|---|---|---|
| 5′ ITR | 2076 | 130 |
| Promoter | 2239 | 380 |
| Intron | 2240 | 566 |
| Signal | 1740 | 57 |
| VH | 1821 | 351 |
| Constant | 1864 | 321 |
| Linker | 2251 | 90 |
| VL | 1939 | 336 |
| Tag | 2118 | 27 |
| PolyA | 2122 | 127 |
| 3′ ITR | 2078 | 130 |

In some embodiments, the AAV particle genome comprises SEQ ID NO: 2186 (TAU_ITR136) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a light chain constant region, a (G4S)6 linker (“(G4S)4” disclosed as SEQ ID NO: 4541), an IPN002 antibody light chain variable region, an HA tag, and a rabbit globin polyadenylation signal sequence.

TABLE 62
Sequence Regions in ITR to ITR Sequences

| | |
|---|---|
| Sequence | TAU_ITR121 (SEQ ID NO: 2171) |

| Regions | Region SEQ ID NO | Region length |
|---|---|---|
| 5′ ITR | 2076 | 130 |
| Promoter | 2239 | 380 |
| Intron | 2240 | 566 |
| Signal | 2241 | 57 |
| VH | 1821 | 351 |
| Constant | 2242 | 309 |
| Linker | 1724 | 12 |
| Linker | 1726 | 54 |
| Constant | 1864 | 321 |
| PolyA | 2122 | 127 |
| 3′ ITR | 2078 | 130 |

In some embodiments, the AAV particle genome comprises SEQ ID NO: 2171 (TAU_ITR121) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a heavy chain constant region, a furin cleavage site, a T2A linker, a light chain constant region, and a rabbit globin polyadenylation signal sequence.

TABLE 63
Sequence Regions in ITR to ITR Sequences

| | |
|---|---|
| TAU_ITR123 | TAU_ITR124 |
| (SEQ ID NO: 2173) | (SEQ ID NO: 2174) |

| Sequence | Region SEQ | Region | Region SEQ | Region |
|---|---|---|---|---|
| Regions | ID NO | length | ID NO | length |
| 5′ ITR | 2076 | 130 | 2076 | 130 |
| Promoter | 2239 | 380 | 2239 | 380 |
| Intron | 2240 | 566 | 2240 | 566 |
| Signal | 2241 | 57 | 2241 | 57 |
| VH | 1821 | 351 | 1821 | 351 |
| Constant | 2243 | 291 | 2242 | 309 |
| Linker | 1724 | 12 | 1724 | 12 |
| Linker | 1726 | 54 | 1726 | 54 |
| Signal | 1861 | 57 | 1861 | 57 |
| VL | 1939 | 336 | 1939 | 336 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 2173 (TAU_ITR123) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a heavy chain constant region, a furin cleavage site, a T2A linker, a light chain signal sequence, an IPN002 antibody light chain variable region, a light chain constant region, and a rabbit globin polyadenylation signal sequence.

In some embodiments, the AAV particle genome comprises SEQ ID NO: 2174 (TAU_ITR124) which comprises a 5′ inverted terminal repeat (ITR) sequence region and a 3′ ITR sequence region, a CMVie region, a human beta-globin intron region, a heavy chain signal sequence, an IPN002 antibody heavy chain variable region, a heavy chain constant region, a furin cleavage site, a T2A linker, a light chain signal sequence, an IPN002 antibody light chain variable region, a light chain constant region, and a rabbit globin polyadenylation signal sequence.

TABLE 64
Sequence Regions in ITR to ITR Sequences

| | TAU_ITR145 | | TAU_ITR146 | | TAU_ITR147 | | TAU_ITR148 | | TAU_ITR149 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2195) | | 2196) | | 2197) | | 2198) | | 2199) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2076 | 130 | 2076 | 130 | 2076 | 130 | 2076 | 130 |
| Promoter | 2239 | 380 | 2239 | 380 | 2239 | 380 | 2239 | 380 | 2239 | 380 |
| Intron | 2240 | 566 | 2240 | 566 | 2240 | 566 | 2240 | 566 | 2240 | 566 |
| Signal | 1861 | 57 | 1861 | 57 | 1861 | 57 | 1861 | 57 | 1861 | 57 |
| VL | 1939 | 336 | 1939 | 336 | 1939 | 336 | 1939 | 336 | 1939 | 336 |
| Linker | 2246 | 30 | 1730 | 45 | 2254 | 60 | 2249 | 75 | 2251 | 90 |
| VH | 1821 | 351 | 1821 | 351 | 1821 | 351 | 1821 | 351 | 1821 | 351 |
| Tag | 2118 | 27 | 2118 | 27 | 2118 | 27 | 2118 | 27 | 2118 | 27 |
| PolyA | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2195 (TAU_ITR145) which comprises a 5′ inverted terminal repeat (ITR) sequence region and a 3′ ITR sequence region, a CMVie region, a human beta-globin intron region, a light chain signal sequence, an IPN002 antibody light chain variable region, a (G4S)2 linker (“(G4S)4” disclosed as SEQ ID NO: 4539), an IPN002 antibody heavy chain variable region, an HA tag, and a rabbit globin polyadenylation signal sequence.

In some embodiments, the AAV particle genome comprises SEQ ID NO: 2196 (TAU_ITR146) which comprises a 5′ inverted terminal repeat (ITR) sequence region and a 3′ ITR sequence region, a CMVie region, a human beta-globin intron region, a light chain signal sequence, an IPN002 antibody light chain variable region, a (G4S)3 linker (“(G4S)3” disclosed as SEQ ID NO: 4537), an IPN002 antibody heavy chain variable region, an HA tag, and a rabbit globin polyadenylation signal sequence.

In some embodiments, the AAV particle genome comprises SEQ ID NO: 2197 (TAU_ITR147) which comprises a 5′ inverted terminal repeat (ITR) sequence region and a 3′ ITR sequence region, a CMVie region, a human beta-globin intron region, a light chain signal sequence, an IPN002 antibody light chain variable region, a (G4S)4 linker (“(G4S)4” disclosed as SEQ ID NO: 4540), an IPN002 antibody heavy chain variable region, an HA tag, and a rabbit globin polyadenylation signal sequence.

In some embodiments, the AAV particle genome comprises SEQ ID NO: 2198 (TAU_ITR148) which comprises a 5′ inverted terminal repeat (ITR) sequence region and a 3′ ITR sequence region, a CMVie region, a human beta-globin intron region, a light chain signal sequence, an IPN002 antibody light chain variable region, a (G4S)5 linker (“(G4S)5” disclosed as SEQ ID NO: 4538), an IPN002 antibody heavy chain variable region, an HA tag, and a rabbit globin polyadenylation signal sequence.

In some embodiments, the AAV particle genome comprises SEQ ID NO: 2199 (TAU_ITR149) which comprises a 5′ inverted terminal repeat (ITR) sequence region and a 3′ ITR sequence region, a CMVie region, a human beta-globin intron region, a light chain signal sequence, an IPN002 antibody light chain variable region, a (G4S)6 linker (“(G4S)4” disclosed as SEQ ID NO: 4541), an IPN002 antibody heavy chain variable region, an HA tag, and a rabbit globin polyadenylation signal sequence.

TABLE 65
Sequence Regions in ITR to ITR Sequences

| | |
|---|---|
| Sequence | TAU_ITR244 (SEQ ID NO: 2316) |

| Regions | Region SEQ ID NO | Region length |
|---|---|---|
| 5′ ITR | 2076 | 130 |
| CAG promoter | 2080 | 1715 |
| CMVie | 2081 | 299 |
| CBA min. | 2082 | 283 |
| VH | 1838 | 1377 |
| Linker | 1724 | 12 |
| Linker | 1726 | 54 |
| VL | 1936 | 720 |
| PolyA | 2122 | 127 |
| 3′ ITR | 2078 | 130 |

In some embodiments, the AAV particle genome comprises SEQ ID NO: 2316 (TAU_ITR244) which comprises a 5′ inverted terminal repeat (ITR) sequence region and a 3′ ITR sequence region, a CAG promoter comprising a CMVie region and a minimal CBA promoter region, a PHF1 antibody heavy chain variable region, a furin cleavage site, a T2A linker, a PHF1 antibody light chain variable region and a rabbit beta globin polyadenylation signal sequence.

TABLE 66
Sequence Regions in ITR to ITR Sequences

| | |
|---|---|
| Sequence | TAU_ITR248 (SEQ ID NO: 2320) |

| Regions | Region SEQ ID NO | Region length |
|---|---|---|
| 5′ ITR | 2077 | 141 |
| CAG promoter | 2080 | 1715 |
| CMVie | 2081 | 299 |
| CBA min. | 2082 | 283 |
| VH | 2169 | 435 |
| Linker | 1730 | 45 |
| VL | 1957 | 339 |
| PolyA | 2123 | 477 |
| Filler | 2126 | 1240 |
| 3′ ITR | 2079 | 141 |

In some embodiments, the AAV particle genome comprises SEQ ID NO: 2320 (TAU_ITR248) which comprises a 5′ inverted terminal repeat (ITR) sequence region and a 3′ ITR sequence region, a CAG promoter comprising a CMVie region and a minimal CBA promoter region, a PHF1 antibody heavy chain variable region, a furin cleavage site, a PHF1 antibody light chain variable region, a rabbit beta globin polyadenylation signal sequence and a human albumin derived filler sequence.

TABLE 67
Sequence Regions in ITR to ITR Sequences

| | TAU_ITR243 | | TAU_ITR242 | | TAU_ITR241 | |
|---|---|---|---|---|---|---|
| | (SEQ ID NO: | | (SEQ ID NO: | | (SEQ ID NO: | |
| | 2315) | | 2314) | | 2313) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| Promoter | 2084 | 654 | 2085 | 699 | 5086 | 557 |
| Intron | 2240 | 566 | 2240 | 566 | 2240 | 566 |
| VH | 1838 | 1377 | 1838 | 1377 | 1838 | 1377 |
| Linker | 1724 | 12 | 1724 | 12 | 1724 | 12 |
| Linker | 1726 | 54 | 1726 | 54 | 1726 | 54 |
| VL | 1936 | 720 | 1936 | 720 | 1936 | 720 |
| PolyA | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2315 (TAU_ITR243) which comprises a 5′ inverted terminal repeat (ITR) sequence region and a 3′ ITR sequence region, a CBA promoter, a human beta-globin intron region, a PHF1 antibody heavy chain variable region, a furin cleavage site, a T2A linker, an PHF1 antibody light chain variable region, and a rabbit globin polyadenylation sequence.

In some embodiments, the AAV particle genome comprises SEQ ID NO: 2314 (TAU_ITR242) which comprises a 5′ inverted terminal repeat (ITR) sequence region and a 3′ ITR sequence region, a GFAP promoter, a human beta-globin intron region, a PHF1 antibody heavy chain variable region, a furin cleavage site, a T2A linker, an PHF1 antibody light chain variable region, and a rabbit globin polyadenylation sequence.

In some embodiments, the AAV particle genome comprises SEQ ID NO: 2313 (TAU_ITR241) which comprises a 5′ inverted terminal repeat (ITR) sequence region and a 3′ ITR sequence region, a synapsin promoter, a human beta-globin intron region, a PHF1 antibody heavy chain variable region, a furin cleavage site, a T2A linker, an PHF1 antibody light chain variable region, and a rabbit globin polyadenylation sequence.

TABLE 68
Sequence Regions in ITR to ITR Sequences

| | TAU_ITR245 | | TAU_ITR246 | | TAU_ITR247 | |
|---|---|---|---|---|---|---|
| | (SEQ ID NO: | | (SEQ ID NO: | | (SEQ ID NO: | |
| | 2317) | | 2318) | | 2319) | |
| | Region | | Region | | Region | |
| Sequence | SEQ | Region | SEQ | Region | SEQ | Region |
| Regions | ID NO | length | ID NO | length | ID NO | length |
| 5′ ITR | 2077 | 141 | 2077 | 141 | 2077 | 141 |
| CBA | 2084 | 654 | 2084 | 654 | 2084 | 654 |
| Promoter | | | | | | |
| Intron | 2256 | 491 | 2257 | 387 | 2258 | 292 |
| VH | 2170 | 408 | 2170 | 408 | 2170 | 408 |
| Linker | 1730 | 45 | 1730 | 45 | 1730 | 45 |
| VL | 1957 | 339 | 1957 | 339 | 1957 | 339 |
| HA Tag | 2118 | 27 | 2118 | 27 | 2118 | 27 |
| PolyA | 2122 | 127 | 2122 | 127 | 2122 | 127 |
| 3′ ITR | 2079 | 141 | 2079 | 141 | 2079 | 141 |

In some embodiments, the AAV particle genome comprises SEQ ID NO: 2317 (TAU_ITR245) which comprises a 5′ inverted terminal repeat (ITR) sequence region and a 3′ ITR sequence region, a CBA promoter, a human beta-globin intron region, a PHF1 antibody heavy chain variable region, a furin cleavage site, an PHF1 antibody light chain variable region, a HA tag and a rabbit globin polyadenylation sequence.

In some embodiments, the AAV particle genome comprises SEQ ID NO: 2318 (TAU_ITR246) which comprises a 5′ inverted terminal repeat (ITR) sequence region and a 3′ ITR sequence region, a CBA promoter, a human beta-globin intron region, a PHF1 antibody heavy chain variable region, a furin cleavage site, an PHF1 antibody light chain variable region, a HA tag and a rabbit globin polyadenylation sequence.

In some embodiments, the AAV particle genome comprises SEQ ID NO: 2319 (TAU_ITR247) which comprises a 5′ inverted terminal repeat (ITR) sequence region and a 3′ ITR sequence region, a CBA promoter, a human beta-globin intron region, a PHF1 antibody heavy chain variable region, a furin cleavage site, an PHF1 antibody light chain variable region, a HA tag and a rabbit globin polyadenylation sequence.

TABLE 69
Sequence Regions in ITR to ITR Sequences

| | |
|---|---|
| Sequence | TAU_ITR249 (SEQ ID NO: 2321) |

| Regions | Region SEQ ID NO | Region length |
|---|---|---|
| 5′ ITR | 2077 | 141 |
| CBA promoter | 2084 | 654 |
| VH | 2169 | 435 |
| Linker | 1730 | 45 |
| VL | 1957 | 339 |
| PolyA | 2123 | 477 |
| Filler | 2126 | 1240 |
| 3′ ITR | 2079 | 141 |

In some embodiments, the AAV particle genome comprises SEQ ID NO: 2321 (TAU_ITR249) which comprises a 5′ inverted terminal repeat (ITR) sequence region and a 3′ ITR sequence region, a CBA promoter, a PHF1 antibody heavy chain variable region, a furin cleavage site, a PHF1 antibody light chain variable region, a rabbit beta globin polyadenylation signal sequence and a human albumin derived filler sequence.

TABLE 70
Sequence Regions in ITR to ITR Sequences

| | TAU_ITR158 | | TAU_ITR170 | | TAU_ITR171 | | TAU_ITR172 | |
|---|---|---|---|---|---|---|---|---|
| | (SEQ ID NO: | | (SEQ ID NO: | | (SEQ ID NO: | | (SEQ ID NO: | |
| | 2208) | | 2220) | | 2221) | | 2222) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| CAG | 2080 | 1715 | 2080 | 1715 | 2080 | 1715 | 2080 | 1715 |
| promoter | | | | | | | | |
| CMVie | 2081 | 299 | 2081 | 299 | 2081 | 299 | 2081 | 299 |
| CBA min. | 2082 | 283 | 2082 | 283 | 2082 | 283 | 2082 | 283 |
| Signal | 1740 | 57 | 1740 | 57 | 1740 | 57 | 1740 | 57 |
| VH | 1859 | 354 | 2241 | 348 | 2241 | 348 | 1859 | 354 |
| Linker | 2247 | 45 | 2247 | 45 | 2247 | 45 | 2247 | 45 |
| VL | 1980 | 321 | 1980 | 321 | 1980 | 321 | 1980 | 321 |
| HA Tag | 2118 | 27 | 2118 | 27 | 2118 | 27 | 2118 | 27 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2208 (TAU_ITR158) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence.

In some embodiments, the AAV particle genome comprises SEQ ID NO: 2220 (TAU_ITR170) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence.

In some embodiments, the AAV particle genome comprises SEQ ID NO: 2221 (TAU_ITR171) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence.

In some embodiments, the AAV particle genome comprises SEQ ID NO: 2222 (TAU_ITR172) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence

TABLE 71
Sequence Regions in ITR to ITR Sequences

| | TAU_ITR173 | | TAU_ITR174 | | TAU_ITR175 | | TAU_ITR179 | |
|---|---|---|---|---|---|---|---|---|
| | (SEQ ID NO: | | (SEQ ID NO: | | (SEQ ID NO: | | (SEQ ID NO: | |
| | 2223) | | 2224) | | 2225) | | 2229) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| CAG | 2080 | 1715 | 2080 | 1715 | 2080 | 1715 | 2080 | 1715 |
| promoter | | | | | | | | |
| CMVie | 2081 | 299 | 2081 | 299 | 2081 | 299 | 2081 | 299 |
| CBA min. | 2082 | 283 | 2082 | 283 | 2082 | 283 | 2082 | 283 |
| Signal | 1740 | 57 | 1740 | 57 | 1740 | 57 | 1740 | 57 |
| VH | 1859 | 354 | 1859 | 354 | 1859 | 354 | 1859 | 354 |
| Linker | 2247 | 45 | 2247 | 45 | 2247 | 45 | 2247 | 45 |
| VL | 1980 | 321 | 1980 | 321 | 1980 | 321 | 1980 | 321 |
| HA Tag | 2118 | 27 | 2118 | 27 | 2118 | 27 | 2118 | 27 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2223 (TAU_ITR173) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence.

In some embodiments, the AAV particle genome comprises SEQ ID NO: 2224 (TAU_ITR174) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence.

In some embodiments, the AAV particle genome comprises SEQ ID NO: 2225 (TAU_ITR175) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence.

In some embodiments, the AAV particle genome comprises SEQ ID NO: 2229 (TAU_ITR179) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)3 linker, a light chain signal, a PT3 antibody light chain variable region, a HA tag, and a rabbit beta globin polyadenylation signal sequence.

TABLE 72
Sequence Regions in ITR to ITR Sequences

| | TAU_ITR157 | | TAU_ITR159 | |
|---|---|---|---|---|
| | (SEQ ID NO: 2207) | | (SEQ ID NO: 2209) | |
| Sequence | Region SEQ | Region | Region SEQ | Region |
| Regions | ID NO | length | ID NO | length |
| 5′ ITR | 2076 | 130 | 2076 | 130 |
| CAG promoter | 2080 | 1715 | 2080 | 1715 |
| CMVie | 2081 | 299 | 2081 | 299 |
| CBA min. | 2082 | 283 | 2082 | 283 |
| Signal | 1740 | 57 | 1740 | 57 |
| VH | 1859 | 354 | 1859 | 354 |
| Linker | 2246 | 30 | 2248 | 60 |
| VL | 1980 | 321 | 1980 | 321 |
| HA Tag | 2118 | 27 | 2118 | 27 |
| PolyA | 2122 | 127 | 2122 | 127 |
| 3′ ITR | 2078 | 130 | 2078 | 130 |

In some embodiments, the AAV particle genome comprises SEQ ID NO: 2207 (TAU_ITR157) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)2 linker, a light chain signal, a PT3 antibody light chain variable region, a HA tag, and a rabbit beta globin polyadenylation signal sequence.

In some embodiments, the AAV particle genome comprises SEQ ID NO: 2209 (TAU_ITR159) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable region, a (G4S)4 linker, a light chain signal, a PT3 antibody light chain variable region, a HA tag, and a rabbit beta globin polyadenylation signal sequence.

TABLE 73
Sequence Regions in ITR to ITR Sequences

| | TAU_ITR104 | | TAU_ITR181 | |
|---|---|---|---|---|
| | (SEQ ID NO: 2154) | | (SEQ ID NO: 2231) | |
| Sequence | Region SEQ | Region | Region SEQ | Region |
| Regions | ID NO | length | ID NO | length |
| 5′ ITR | 2076 | 130 | 2076 | 130 |
| CAG promoter | 2080 | 1715 | 2080 | 1715 |
| CMVie | 2081 | 299 | 2081 | 299 |
| CBA min. | 2082 | 283 | 2082 | 283 |
| Signal | 1740 | 57 | 1740 | 57 |
| VH | 1859 | 354 | 1859 | 354 |
| Constant | 1742 | 969 | 2243 | 291 |
| Linker | 1725 | 12 | 1725 | 12 |
| Linker | 1727 | 75 | 1727 | 75 |
| Signal | 1861 | 57 | 1861 | 57 |
| VL | 1980 | 321 | 1980 | 321 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 2154 (TAU_ITR104) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2231 (TAU_ITR181) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

TABLE 74
Sequence Regions in ITR to ITR Sequences

| | TAU_ITR112 | | TAU_ITR120 | | TAU_ITR180 | | TAU_ITR182 | |
|---|---|---|---|---|---|---|---|---|
| | (SEQ ID NO: | | (SEQ ID NO: | | (SEQ ID NO: | | (SEQ ID NO: | |
| | 2162) | | 2162) | | 2230) | | 2232) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| CAG | 2080 | 1715 | 2080 | 1715 | 2080 | 1715 | 2080 | 1715 |
| promoter | | | | | | | | |
| CMVie | 2081 | 299 | 2081 | 299 | 2081 | 299 | 2081 | 299 |
| CBA min. | 2082 | 283 | 2082 | 283 | 2082 | 283 | 2082 | 283 |
| Signal | 1740 | 57 | 1740 | 57 | 1740 | 57 | 1740 | 57 |
| VH | 1859 | 354 | 1859 | 354 | 1859 | 354 | 1859 | 354 |
| Constant | 1742 | 969 | 1742 | 969 | 2243 | 291 | 2242 | 309 |
| Linker | 1724 | 12 | 1724 | 12 | 1724 | 12 | 1724 | 12 |
| Linker | 1726 | 54 | 1726 | 54 | 1726 | 54 | 1726 | 54 |
| Signal | 1861 | 57 | 1861 | 57 | 1861 | 57 | 1861 | 57 |
| VL | 1980 | 321 | 1980 | 321 | 1980 | 321 | 1980 | 321 |
| Constant | 1864 | 321 | 1864 | 321 | 1864 | 321 | 1864 | 321 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2162 (TAU_ITR112) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

In some embodiments, the AAV particle genome comprises SEQ ID NO: 2162 (TAU_ITR120) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

In some embodiments, the AAV particle genome comprises SEQ ID NO: 2230 (TAU_ITR180) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

In some embodiments, the AAV particle genome comprises SEQ ID NO: 2232 (TAU_ITR182) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

TABLE 75
Sequence Regions in ITR to ITR Sequences

| | TAU_ITR193 | | TAU_ITR190 | |
|---|---|---|---|---|
| | (SEQ ID NO: 2265) | | (SEQ ID NO: 2262) | |
| Sequence | Region SEQ | Region | Region SEQ | Region |
| Regions | ID NO | length | ID NO | length |
| 5′ ITR | 2076 | 130 | 2076 | 130 |
| CAG promoter | 2080 | 1715 | 2080 | 1715 |
| CMVie | 2081 | 299 | 2081 | 299 |
| CBA min. | 2082 | 283 | 2082 | 283 |
| Signal | 1740 | 57 | 1740 | 57 |
| VH | 1859 | 354 | 1859 | 354 |
| Constant | 2242 | 309 | 2242 | 309 |
| Hinge | 1737 | 198 | 1737 | 198 |
| Linker | 1724 | 12 | 1724 | 12 |
| Linker | 1727 | 75 | 1726 | 54 |
| Signal | 1861 | 57 | 1861 | 57 |
| VL | 1980 | 321 | 1980 | 321 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 2265 (TAU_ITR193) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a hinge region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2262 (TAU_ITR190) which comprises a 5′ inverted terminal repeat (ITR) sequence region and a 3′ ITR sequence region, a CAG promoter comprising a CMVie region and a minimal CBA promoter region, a heavy chain signal, a PT3 antibody heavy chain variable and constant region, a hinge region, a furin cleavage site, a T2A linker, a light chain signal, a PT3 antibody light chain variable and constant region and a rabbit beta globin polyadenylation signal sequence.

TABLE 76
Sequence Regions in ITR to ITR Sequences

| | |
|---|---|
| Sequence | TAU_ITR156 (SEQ ID NO: 2206) |

| Regions | Region SEQ ID NO | Region length |
|---|---|---|
| 5′ ITR | 2076 | 130 |
| CAG promoter | 2080 | 1715 |
| CMVie | 2081 | 299 |
| CBA min. | 2082 | 283 |
| Signal | 1861 | 57 |
| VH | 1859 | 354 |
| Linker | 2247 | 45 |
| VL | 1980 | 321 |
| HA Tag | 2118 | 27 |
| PolyA | 2122 | 127 |
| 3′ ITR | 2078 | 130 |

In some embodiments, the AAV particle genome comprises SEQ ID NO: 2206 (TAU_ITR156) which comprises a 5′ inverted terminal repeat (ITR) sequence region and a 3′ ITR sequence region, a CAG promoter comprising a CMVie region and a minimal CBA promoter region, a light chain signal, a PT3 antibody heavy chain variable region, a (G4S)3 linker, a PT3 antibody light chain variable region, a HA tag and a rabbit beta globin polyadenylation signal sequence.

TABLE 77
Sequence Regions in ITR to ITR Sequences

| | |
|---|---|
| Sequence | TAU_ITR160 (SEQ ID NO: 2210) |

| Regions | Region SEQ ID NO | Region length |
|---|---|---|
| 5′ ITR | 2076 | 130 |
| CAG promoter | 2080 | 1715 |
| CMVie | 2081 | 299 |
| CBA min. | 2082 | 283 |
| Signal | 1740 | 57 |
| VL | 1980 | 321 |
| Linker | 2247 | 45 |
| VH | 1859 | 354 |
| HA Tag | 2118 | 27 |
| PolyA | 2122 | 127 |
| 3′ ITR | 2078 | 130 |

In some embodiments, the AAV particle genome comprises SEQ ID NO: 2210 (TAU_ITR160) which comprises a 5′ inverted terminal repeat (ITR) sequence region and a 3′ ITR sequence region, a CAG promoter comprising a CMVie region and a minimal CBA promoter region, a heavy chain signal, a PT3 antibody light chain variable region, a (G4S)3 linker, a PT3 antibody heavy chain variable region, a HA tag and a rabbit beta globin polyadenylation signal sequence.

TABLE 78
Sequence Regions in ITR to ITR Sequences

| | TAU_ITR161 | | TAU_ITR162 | | TAU_ITR196 | | TAU_ITR163 | |
|---|---|---|---|---|---|---|---|---|
| | (SEQ ID NO: | | (SEQ ID NO: | | (SEQ ID NO: | | (SEQ ID NO: | |
| | 2211) | | 2212) | | 2268) | | 2213) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| CAG | 2080 | 1715 | 2080 | 1715 | 2080 | 1715 | 2080 | 1715 |
| promoter | | | | | | | | |
| CMVie | 2081 | 299 | 2081 | 299 | 2081 | 299 | 2081 | 299 |
| CBA min. | 2082 | 283 | 2082 | 283 | 2082 | 283 | 2082 | 283 |
| Signal | 1861 | 57 | 1861 | 57 | 1861 | 57 | 1861 | 57 |
| VL | 1980 | 321 | 1980 | 321 | 1980 | 321 | 1980 | 321 |
| Linker | 2246 | 30 | 2247 | 45 | 2247 | 45 | 2248 | 60 |
| VH | 1859 | 354 | 1859 | 354 | 1859 | 354 | 1859 | 354 |
| HA Tag | 2118 | 27 | 2118 | 27 | 2255 | 18 | 2118 | 27 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 2211 (TAU_ITR161) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable region, a (G4S)2 linker, a PT3 antibody heavy chain variable region, a HA tag and a rabbit beta globin polyadenylation signal sequence.

In some embodiments, the AAV particle genome comprises SEQ ID NO: 2212 (TAU_ITR162) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable region, a (G4S)3 linker, a PT3 antibody heavy chain variable region, a HA tag and a rabbit beta globin polyadenylation signal sequence.

In some embodiments, the AAV particle genome comprises SEQ ID NO: 2268 (TAU_ITR196) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable region, a (G4S)3 linker, a PT3 antibody heavy chain variable region, a HA tag and a rabbit beta globin polyadenylation signal sequence.

In some embodiments, the AAV particle genome comprises SEQ ID NO: 2213 (TAU_ITR163) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable region, a (G4S)4 linker, a PT3 antibody heavy chain variable region, a HA tag and a rabbit beta globin polyadenylation signal sequence.

TABLE 79
Sequence Regions in ITR to ITR Sequences

| | TAU_ITR108 | | TAU_ITR184 | | TAU_ITR116 | | TAU_ITR183 | | TAU_ITR185 | |
|---|---|---|---|---|---|---|---|---|---|---|
| | (SEQ ID NO: | | (SEQ ID NO: | | (SEQ ID NO: | | (SEQ ID NO: | | (SEQ ID NO: | |
| | 2158) | | 2234) | | 2166) | | 2233) | | 2235) | |
| | Region | | Region | | Region | | Region | | Region | |
| Sequence | SEQ | Region | SEQ | Region | SEQ | Region | SEQ | Region | SEQ | Region |
| Regions | ID NO | length | ID NO | length | ID NO | length | ID NO | length | ID NO | length |
| 5′ ITR | 2076 | 130 | 2676 | 130 | 2076 | 130 | 2076 | 130 | 2076 | 130 |
| CAG | 2080 | 1715 | 2080 | 1715 | 2080 | 1715 | 2080 | 1715 | 2080 | 1715 |
| promoter | | | | | | | | | | |
| CMVie | 2081 | 299 | 2081 | 299 | 2081 | 299 | 2081 | 299 | 2081 | 299 |
| CBA min. | 2082 | 283 | 2082 | 283 | 2082 | 283 | 2082 | 283 | 2082 | 283 |
| Signal | 1861 | 57 | 1861 | 57 | 1861 | 57 | 1861 | 57 | 1861 | 57 |
| VL | 1980 | 321 | 1980 | 321 | 1980 | 321 | 1980 | 321 | 1980 | 321 |
| Constant | 1864 | 321 | 1864 | 321 | 1864 | 321 | 1864 | 321 | 1864 | 321 |
| Linker | 1727 | 75 | 1727 | 75 | 1726 | 54 | 1726 | 54 | 1726 | 54 |
| Signal | 1740 | 57 | 1740 | 57 | 2241 | 57 | 1740 | 57 | 1740 | 57 |
| VH | 1859 | 354 | 1859 | 354 | 1859 | 354 | 1859 | 354 | 1859 | 354 |
| Constant | 1742 | 969 | 2243 | 291 | 1742 | 969 | 2243 | 291 | 2242 | 309 |
| Poly A | 2122 | 127 | 2122 | 127 | 2122 | 127 | 2122 | 127 | 2122 | 127 |
| 3′ ITR | 2078 | 130 | 2078 | 130 | 2078 | 130 | 2078 | 130 | 2078 | 130 |

In some embodiments, the AAV particle genome comprises SEQ ID NO: 2158 (TAU_ITR108) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable and constant region, a F2A linker, a heavy chain signal, a PT3 antibody heavy chain variable and constant region and a rabbit beta globin polyadenylation signal sequence.

In some embodiments, the AAV particle genome comprises SEQ ID NO: 2234 (TAU_ITR184) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable and constant region, a F2A linker, a heavy chain signal, a PT3 antibody heavy chain variable and constant region and a rabbit beta globin polyadenylation signal sequence.

In some embodiments, the AAV particle genome comprises SEQ ID NO: 2166 (TAU_ITR116) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable and constant region, a T2A linker, a heavy chain signal, a PT3 antibody heavy chain variable and constant region and a rabbit beta globin polyadenylation signal sequence.

In some embodiments, the AAV particle genome comprises SEQ ID NO: 2233 (TAU_ITR183) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable and constant region, a T2A linker, a heavy chain signal, a PT3 antibody heavy chain variable and constant region and a rabbit beta globin polyadenylation signal sequence.

In some embodiments, the AAV particle genome comprises SEQ ID NO: 2235 (TAU_ITR185) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable and constant region, a T2A linker, a heavy chain signal, a PT3 antibody heavy chain variable and constant region and a rabbit beta globin polyadenylation signal sequence.

TABLE 80
Sequence Regions in ITR to ITR Sequences

| | TAU_ITR186 | | TAU_ITR187 | |
|---|---|---|---|---|
| | (SEQ ID NO: 2236) | | (SEQ ID NO: 2237) | |
| Sequence | Region SEQ | Region | Region SEQ | Region |
| Regions | ID NO | length | ID NO | length |
| 5′ ITR | 2076 | 130 | 2076 | 130 |
| CAG promoter | 2080 | 1715 | 2080 | 1715 |
| CMVie | 2081 | 299 | 2081 | 299 |
| CBA min. | 2082 | 283 | 2082 | 283 |
| Signal | 1861 | 57 | 1861 | 57 |
| VL | 1980 | 321 | 1980 | 321 |
| Constant | 1864 | 321 | 1864 | 321 |
| Linker | 1724 | 12 | 1724 | 12 |
| Linker | 1726 | 54 | 1726 | 54 |
| Signal | 1740 | 57 | 1740 | 57 |
| VH | 1859 | 354 | 1859 | 354 |
| Constant | 2243 | 291 | 2242 | 309 |
| PolyA | 2122 | 127 | 2122 | 127 |
| 3′ ITR | 2078 | 130 | 2078 | 130 |

In some embodiments, the AAV particle genome comprises SEQ ID NO: 2236 (TAU_ITR186) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable and constant region, a furin cleavage site, a T2A linker, a heavy chain signal, a PT3 antibody heavy chain variable and constant region and a rabbit beta globin polyadenylation signal sequence.

In some embodiments, the AAV particle genome comprises SEQ ID NO: 2237 (TAU_ITR187) which comprises a 5′ inverted terminal repeat (ITR) sequence region and a 3′ ITR sequence region, a CAG promoter comprising a CMVie region and a minimal CBA promoter region, a light chain signal, a PT3 antibody light chain variable and constant region, a furin cleavage site, a T2A linker, a heavy chain signal, a PT3 antibody heavy chain variable and constant region and a rabbit beta globin polyadenylation signal sequence.

TABLE 81
Sequence Regions in ITR to ITR Sequences

| | TAU_ITR176 | | TAU_ITR177 | | TAU_ITR178 | |
|---|---|---|---|---|---|---|
| | (SEQ ID NO: | | (SEQ ID NO: | | (SEQ ID NO: | |
| | 2226) | | 2227) | | 2228) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| CAG | 2080 | 1715 | 2080 | 1715 | 2080 | 1715 |
| promoter | | | | | | |
| CMVie | 2081 | 299 | 2081 | 299 | 2081 | 299 |
| CBA mm. | 2082 | 283 | 2082 | 283 | 2082 | 283 |
| VH | 1859 | 354 | 1859 | 354 | 1859 | 354 |
| Linker | 2247 | 45 | 2247 | 45 | 2247 | 45 |
| VL | 1980 | 321 | 1980 | 321 | 1980 | 321 |
| HA Tag | 2118 | 37 | 2118 | 27 | 2118 | 27 |
| Poly A | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2226 (TAU_ITR176) which comprises a 5′ inverted terminal repeat (ITR) sequence region and a 3′ ITR sequence region, a CAG promoter comprising a CMVie region and a minimal CBA promoter region, a PT3 antibody heavy chain variable region, a (G4S)3 linker, a PT3 antibody light chain variable region a HA tag and a rabbit beta globin polyadenylation signal sequence.

In some embodiments, the AAV particle genome comprises SEQ ID NO: 2227 (TAU_ITR177) which comprises a 5 inverted terminal repeat (ITR) sequence region and a 3′ ITR sequence region, a CAG promoter comprising a CMVie region and a minimal CBA promoter region, a PT3 antibody heavy chain variable region, a (G4S)3 linker, a PT3 antibody light chain variable region, a HA tag and a rabbit beta globin polyadenylation signal sequence.

In some embodiments, the AAV particle genome comprises SEQ ID NO: 2228 (TAU_ITR178) which comprises a 5′ inverted terminal repeat (ITR) sequence region and a 3′ ITR sequence region, a CAG promoter comprising a CMVie region and a minimal CBA promoter region, a PT3 antibody heavy chain variable region, a (G4S)3 linker, a PT3 antibody light chain variable region, a HA tag and a rabbit beta globin polyadenylation signal sequence.

TABLE 82
Sequence Regions in ITR to ITR Sequences

| | TAU_ITR105 | | TAU_ITR113 | | TAU_ITR189 | |
|---|---|---|---|---|---|---|
| | (SEQ ID NO: | | (SEQ ID NO: | | (SEQ ID NO: | |
| | 2155) | | 2163) | | 2261) | |
| | Region | | Region | | Region | |
| Sequence | SEQ | Region | SEQ ID | Region | SEQ ID | Region |
| Regions | ID NO | length | NO | length | NO | length |
| 5′ ITR | 2076 | 130 | 2076 | 130 | 2076 | 130 |
| GFAP | 2085 | 699 | 2085 | 699 | 2085 | 699 |
| Promoter | | | | | | |
| Intron | 2240 | 566 | 2240 | 566 | 2240 | 566 |
| Signal | 1740 | 57 | 1740 | 57 | 1740 | 57 |
| VH | 1859 | 354 | 1859 | 354 | 1859 | 354 |
| Constant | 1742 | 969 | 1742 | 969 | 2243 | 291 |
| Linker | 1725 | 12 | 1724 | 12 | 1724 | 12 |
| Linker | 1727 | 75 | 1726 | 54 | 1726 | 54 |
| Signal | 1980 | 321 | 1980 | 321 | 1980 | 321 |
| VL | 1864 | 321 | 1864 | 321 | 1864 | 321 |
| Poly A | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2155 (TAU_ITR105) which comprises a 5′ inverted terminal repeat (ITR) sequence region and a 3′ ITR sequence region, a GFAP promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2163 (TAU_ITR113) which comprises a 5′ inverted terminal repeat (ITR) sequence region and a 3′ ITR sequence region, a GFAP promoter, a human beta-globin intron region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

In some embodiments, the AAV particle genome comprises SEQ ID NO: 2261 (TAU_ITR189) which comprises a 5′ inverted terminal repeat (ITR) sequence region and a 3′ ITR sequence region, a GFAP promoter, a human beta-globin intron region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

TABLE 83
Sequence Regions in ITR to ITR Sequences

| | TAU_ITR195 | | TAU_ITR192 | |
|---|---|---|---|---|
| | (SEQ ID NO: 2267) | | (SEQ ID NO: 2264) | |
| Sequence | Region SEQ | Region | Region SEQ | Region |
| Regions | ID NO | length | ID NO | length |
| 5′ ITR | 2076 | 130 | 2076 | 130 |
| GFAP Promoter | 2085 | 699 | 2085 | 699 |
| Intron | 2240 | 566 | 2240 | 566 |
| Signal | 1740 | 57 | 1740 | 57 |
| VH | 1859 | 354 | 1859 | 354 |
| Constant | 2242 | 309 | 2242 | 309 |
| Hinge | 1737 | 198 | 1737 | 198 |
| Linker | 1725 | 12 | 1724 | 12 |
| Linker | 1727 | 75 | 1726 | 54 |
| Signal | 1861 | 57 | 1861 | 57 |
| VL | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 2267 (TAU_ITR195) which comprises a 5′ inverted terminal repeat (ITR) sequence region and a 3′ ITR sequence region, a GFAP promoter, a human beta-globin intron region, a heavy chain signal, a PT3 antibody heavy chain variable and constant region, a hinge region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2264 (TAU_ITR192) which comprises a 5′ inverted terminal repeat (ITR) sequence region and a 3′ ITR sequence region, a GFAP promoter, a human beta-globin intron region, a heavy chain signal, a PT3 antibody heavy chain variable and constant region, a hinge region, a furin cleavage site, a T2A linker, a light chain signal, a PT3 antibody light chain variable and constant region and a rabbit beta globin polyadenylation signal sequence.

TABLE 84
Sequence Regions in ITR to ITR Sequences

| | TAU_ITR164 | | TAU_ITR165 | | TAU_ITR166 | |
|---|---|---|---|---|---|---|
| | (SEQ ID NO: | | (SEQ ID NO: | | (SEQ ID NO: | |
| | 2214) | | 2215) | | 2216) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| GFAP | 2085 | 699 | 2085 | 699 | 2085 | 699 |
| Promoter | | | | | | |
| Intron | 2240 | 566 | 2240 | 566 | 2240 | 566 |
| Signal | 1861 | 57 | 1861 | 57 | 1861 | 57 |
| VL | 1980 | 321 | 1980 | 321 | 1980 | 321 |
| Linker | 2246 | 30 | 2247 | 45 | 2248 | 60 |
| VH | 1859 | 354 | 1859 | 354 | 1859 | 354 |
| HA Tag | 2118 | 27 | 2118 | 27 | 2118 | 27 |
| Poly A | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2214 (TAU_ITR164) which comprises a 5′ inverted terminal repeat (ITR) sequence region and a 3′ ITR sequence region, a GFAP promoter, a human beta-globin intron region, a light chain signal, a PT3 antibody light chain variable region, a (G4S)2 linker, a PT3 antibody heavy chain variable region, a HA tag and a rabbit beta globin polyadenylation signal sequence.

In some embodiments, the AAV particle genome comprises SEQ ID NO: 2215 (TAU_ITR165) which comprises a 5′ inverted terminal repeat (ITR) sequence region and a 3′ ITR sequence region, a GFAP promoter, a human beta-globin intron region, a light chain signal, a PT3 antibody light chain variable region, a (G4S)3 linker, a PT3 antibody heavy chain variable region, a HA tag and a rabbit beta globin polyadenylation signal sequence.

In some embodiments, the AAV particle genome comprises SEQ ID NO: 2216 (TAU_ITR166) which comprises a 5′ inverted terminal repeat (ITR) sequence region and a 3′ ITR sequence region a GFAP promoter, a human beta-globin intron region, a light c a 9 signal, a PT3 antibody light chain variable region, a (G4S)4 linker, a PT3 antibody heavy chain variable region, a HA tag and a rabbit beta globin polyadenylation signal sequence.

TABLE 85
Sequence Regions in ITR to ITR Sequences

| | TAU_ITR109 | | TAU_ITR117 | |
|---|---|---|---|---|
| | (SEQ ID NO: 2159) | | (SEQ ID NO: 2167) | |
| Sequence | Region SEQ | Region | Region SEQ | Region |
| Regions | ID NO | length | ID NO | length |
| 5′ ITR | 2076 | 130 | 2076 | 130 |
| GFAP Promoter | 2085 | 699 | 2085 | 699 |
| Intron | 2240 | 566 | 2240 | 566 |
| Signal | 1861 | 57 | 1861 | 57 |
| VL | 1980 | 321 | 1980 | 321 |
| Constant | 1864 | 321 | 1864 | 321 |
| Linker | 1727 | 75 | 1826 | 54 |
| Signal | 1740 | 57 | 2241 | 57 |
| VH | 1859 | 354 | 1859 | 354 |
| Constant | 1742 | 969 | 1742 | 969 |
| PolyA | 2122 | 127 | 2122 | 127 |
| 3′ ITR | 2078 | 130 | 2078 | 130 |

In some embodiments, the AAV particle genome comprises SEQ ID NO: 2159 (TAU_ITR9) which comprises a 5′ inverted terminal repeat (ITR) sequence region and a 3′ ITR sequence region, a GFAP promoter, a human beta-globin intron region, a light chain signal, a 3 antibody light chain variable and constant region, a F2A linker, a heavy chain signal, a PT3 antibody heavy chain variable and constant region and a rabbit beta globin polyadenylation signal sequence.

In some embodiments, the AAV particle genome comprises SEQ ID NO: 2167 (TAU_ITR117) which comprises a 5 inverted terminal repeat (FR) sequence region and a 3′ ITR sequence region, a GFAP promoter, a human beta-globin intron region, a light chain signal, a PT3 antibody light chain variable and constant region, a T2A linker, a heavy chain signal, a PT3 antibody heavy chain variable and constant region and a rabbit beta globin polyadenylation signal sequence.

TABLE 86
Sequence Regions in ITR to ITR Sequences

| | TAU_ITR106 | | TAU_ITR114 | | TAU_ITR188 | |
|---|---|---|---|---|---|---|
| | (SEQ ID NO: | | (SEQ ID NO: | | (SEQ ID NO: | |
| | 2156) | | 2164) | | 2260) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| SYN | 2086 | 557 | 2086 | 557 | 2086 | 557 |
| Promoter | | | | | | |
| Intron | 2240 | 566 | 2240 | 566 | 2240 | 566 |
| Signal | 1740 | 57 | 1740 | 57 | 1740 | 57 |
| VH | 1859 | 354 | 1859 | 354 | 1859 | 354 |
| Constant | 1742 | 969 | 1742 | 969 | 2243 | 291 |
| Linker | 1725 | 12 | 1724 | 12 | 1724 | 12 |
| Linker | 1727 | 75 | 1726 | 54 | 1726 | 54 |
| Signal | 1861 | 57 | 1861 | 57 | 1861 | 57 |
| VL | 1980 | 321 | 1980 | 231 | 1980 | 231 |
| Constant | 1864 | 321 | 1864 | 321 | 1864 | 321 |
| Poly A | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2156 (TAU_ITR106) which comprises a 5′ inverted terminal repeat (ITR) sequence region and a 3′ ITR sequence region, a synapsin promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2164 (TAU_ITR114) which comprises a 5′ inverted terminal repeat (ITR) sequence region and a 3′ ITR sequence region, a synapsin promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2260 (TAU_ITR188) which comprises a 5′ inverted terminal repeat (ITR) sequence region and a 3′ ITR sequence region, a synapsin promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

TABLE 87
Sequence Regions in ITR to ITR Sequences

| | TAU_ITR194 | | TAU_ITR191 | |
|---|---|---|---|---|
| | (SEQ ID NO: 2266) | | (SEQ ID NO: 2263) | |
| Sequence | Region SEQ | Region | Region SEQ | Region |
| Regions | ID NO | length | ID NO | length |
| 5′ ITR | 2076 | 130 | 2076 | 130 |
| SYN Promoter | 2086 | 557 | 2086 | 557 |
| Intron | 2240 | 566 | 2240 | 566 |
| Signal | 1740 | 57 | 1740 | 57 |
| VH | 1859 | 354 | 1859 | 354 |
| Constant | 2242 | 309 | 2242 | 309 |
| Hinge | 1737 | 198 | 1737 | 198 |
| Linker | 1724 | 12 | 1724 | 12 |
| Linker | 1727 | 75 | 1726 | 54 |
| Signal | 1861 | 54 | 1861 | 57 |
| VL | 1980 | 321 | 1980 | 321 |
| Constant | 1864 | 321 | 1864 | 321 |
| PolyA | 2122 | 127 | 2122 | 127 |
| 3′ ITR | 2078 | 130 | 2078 | 130 |

In some embodiments, the AAV particle genome comprises SEQ ID NO: 2266 (TAU_ITR194) which comprises a 5′ inverted terminal repeat (ITR) sequence region and a 3′ ITR sequence region, a synapsin promoter, a human beta-globin intron region, a heavy chain signal, a PT3 antibody heavy chain variable and constant region, a hinge region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2263 (TAU_ITR191) which comprises a 5′ inverted terminal repeat (ITR) sequence region and a 3′ ITR sequence region, a synapsin promoter, a human beta-globin intron region, a heavy chain signal, a PT3 antibody heavy chain variable and constant region, a hinge region, a furin cleavage site, a T2A linker, a light chain signal, a PT3 antibody light chain variable and constant region and a rabbit beta globin polyadenylation signal sequence.

TABLE 88
Sequence Regions in ITR to ITR Sequences

| | TAU_ITR167 | | TAU_ITR168 | | TAU_ITR169 | |
|---|---|---|---|---|---|---|
| | (SEQ ID NO: | | (SEQ ID NO: | | (SEQ ID NO: | |
| | 2217) | | 2218) | | 2219) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| SYN | 2086 | 557 | 2086 | 557 | 2086 | 557 |
| Promoter | | | | | | |
| Intron | 2240 | 566 | 2240 | 566 | 2240 | 566 |
| Signal | 1861 | 57 | 1861 | 57 | 1861 | 57 |
| VL | 1980 | 321 | 1980 | 321 | 1980 | 321 |
| Linker | 2246 | 30 | 2247 | 45 | 2248 | 60 |
| VH | 1859 | 354 | 1859 | 354 | 1859 | 354 |
| HA Tag | 2118 | 27 | 2118 | 27 | 2118 | 27 |
| Poly A | 2122 | 127 | 212.2 | 127 | 2122 | 127 |
| 3′ ITR | 2078 | 130 | 2078 | 130 | 2078 | 130 |

In some embodiments, the AAV particle genome comprises SEQ ID NO: 2217 (TAU_ITR167) which comprises a 5′ inverted terminal repeat (ITR) sequence region and a 3′ ITR sequence region, a synapsin promoter, a human beta-globin intron region, a light chain signal, a PT3 antibody light chain variable region, a (G4S)2 linker, a PT3 antibody heavy chain variable region, a HA tag and a rabbit beta globin polyadenylation signal sequence.

In some embodiments, the AAV particle genome comprises SEQ ID NO: 2218 (TAU_ITR168) which comprises a 5′ inverted terminal repeat (ITR) sequence region and a 3′ ITR sequence region, a synapsin promoter, a human beta-globin intron region, a light chain signal, a PT3 antibody light chain variable region, a (G4S)3 linker, a PT3 antibody heavy chain variable region, a HA tag and a rabbit beta globin polyadenylation signal sequence.

In some embodiments, tbc AAV particle genome comprises SEQ ID NO: 2219 (TAU_ITR169) which comprises a 5′ inverted terminal repeat (ITR) sequence region and a 3′ ITR sequence region, a synapsin promoter, a human beta-globin intron region, a light chain signal, a PT3 antibody light chain variable region, a (G4S)4 linker, a PT3 antibody heavy chain variable region, a HA tag and a rabbit beta globin polyadenylation signal sequence.

TABLE 89
Sequence Regions in ITR to ITR Sequences

| | TAU_ITR110 | | TAU_ITR118 | |
|---|---|---|---|---|
| | (SEQ ID NO: 2160) | | (SEQ ID NO: 2168) | |
| Sequence | Region SEQ | Region | Region SEQ | Region |
| Regions | ID NO | length | ID NO | length |
| 5′ ITR | 2076 | 130 | 2076 | 130 |
| SYN Promoter | 2086 | 557 | 2086 | 557 |
| Intron | 2240 | 566 | 2240 | 566 |
| Signal | 1861 | 57 | 1861 | 57 |
| VL | 1980 | 321 | 1980 | 321 |
| Constant | 1864 | 321 | 1864 | 321 |
| Linker | 1727 | 75 | 1726 | 54 |
| Signal | 1740 | 57 | 2241 | 57 |
| VH | 1859 | 354 | 1859 | 354 |
| Constant | 1742 | 969 | 1742 | 969 |
| PolyA | 2122 | 127 | 2122 | 127 |
| 3′ ITR | 2078 | 130 | 2078 | 130 |

In some embodiments, the AAV particle genome comprises SEQ ID NO: 2160 (TAU_ITR110) which comprises a 5′ inverted terminal repeat (ITR) sequence region and a 3′ ITR sequence region, a synapsin promoter, a human beta-globin intron region, a light chain signal, a PT3 antibody light chain variable region, a F2A linker, a heavy chain signal, a PT3 antibody heavy chain variable and constant region and a rabbit beta globin polyadenylation signal sequence.

In some embodiments, the AAV particle genome comprises SEQ ID NO: 2168 (TAU_ITR18) which comprises a inverted terminal repeat (ITR) sequence region and a 3′ ITR sequence region, a synapsin promoter, a human beta-globin intron region, a light chain signal, a PT3 antibody light chain variable region, a T2A linker, a heavy chain signal, a PT3 antibody heavy chain variable and constant region and a rabbit beta globin polyadenylation signal sequence.

TABLE 90
Sequence Regions in ITR to ITR Sequences

| | TAU_ITR103 | | TAU_ITR111 | | TAU_ITR119 | |
|---|---|---|---|---|---|---|
| | (SEQ ID NO: | | (SEQ ID NO: | | (SEQ ID NO: | |
| | 2153) | | 2161) | | 2161) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| CMVie | 2239 | 380 | 2239 | 380 | 2239 | 380 |
| CB | 2083 | 260 | 2083 | 260 | 2083 | 260 |
| Promoter | | | | | | |
| Intron | 2240 | 566 | 2240 | 566 | 2240 | 566 |
| Signal | 1740 | 57 | 1740 | 57 | 1740 | 57 |
| VH | 1859 | 354 | 1859 | 354 | 1859 | 354 |
| Constant | 1742 | 969 | 1742 | 969 | 1742 | 969 |
| Linker | 1725 | 12 | 1724 | 12 | 1724 | 12 |
| Linker | 1727 | 75 | 1726 | 54 | 1726 | 54 |
| Signal | 1861 | 57 | 1861 | 57 | 1861 | 57 |
| VL | 1980 | 321 | 1980 | 321 | 1980 | 321 |
| Constant | 1864 | 321 | 1864 | 321 | 1864 | 321 |
| Poly A | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2153 (TAU_ITR103) which comprises a 5′ inverted terminal repeat (ITR) sequence region and a 3′ ITR sequence region a CMVie region and a CB promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2161 (TAU_ITR11) which comprises a 5 inverted terminal repeat (ITR) sequence region and a 3′ ITR sequence region, a CMVie region and a CB promoter, a human beta-globin intron region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

In some embodiments, the AAV particle genome comprises SEQ ID NO: 2161 (TAU_ITR119) which comprises a 5′ inverted terminal repeat (ITR) sequence region and a 3′ ITR sequence region, a CMVie region and a CB promoter, a human beta-globin intron region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

TABLE 91
Sequence Regions in ITR to ITR Sequences

| | TAU_ITR107 | | TAU_ITR115 | |
|---|---|---|---|---|
| | (SEQ ID NO: 2157) | | (SEQ ID NO: 2165) | |
| Sequence | Region SEQ | Region | Region SEQ | Region |
| Regions | ID NO | length | ID NO | length |
| 5′ ITR | 2076 | 130 | 2076 | 130 |
| CMVie | 2239 | 380 | 2239 | 380 |
| CB Promoter | 2083 | 260 | 2083 | 260 |
| Intron | 2240 | 566 | 2240 | 566 |
| Signal | 1861 | 57 | 1861 | 57 |
| VL | 1980 | 321 | 1980 | 321 |
| Constant | 1864 | 321 | 1864 | 321 |
| Linker | 1727 | 75 | 1726 | 54 |
| Signal | 1740 | 57 | 2241 | 57 |
| VH | 1859 | 354 | 1859 | 354 |
| Constant | 1742 | 969 | 1742 | 969 |
| PolyA | 2122 | 127 | 2122 | 127 |
| 3′ ITR | 2078 | 130 | 2078 | 130 |

In some embodiments, the AAV particle genome comprises SEQ ID NO: 2157 (TAU_ITR107) which comprises a 5′ inverted terminal repeat (ITR) sequence region and a 3′ ITR sequence region, a CMVie region and a CB promoter, a human beta-globin intron region, a light chain signal, a PT3 antibody light chain variable and constant region, a F2A linker, a heavy chain signal, a PT3 antibody heavy chain variable and constant region and a rabbit beta globin polyadenylation signal sequence.

In some embodiments, the AAV particle genome comprises SEQ ID NO: 2165 (TAU_ITR115) which comprises a 5′ inverted terminal repeat (ITR) sequence region and a 3′ ITR sequence region, a CMVie region and a CB promoter, a human beta-globin intron region, a light chain signal, a PT3 antibody light chain variable and constant region, a F2A linker, a heavy chain signal, a PT3 antibody heavy chain variable and constant region and a rabbit beta globin polyadenylation signal sequence.

In some embodiments, the viral genome may comprise any combination of the following components, including, but not limited to, a 5′ ITR, a promoter region (may comprise one or more component pieces), an intronic region, a Kozak sequence, one or more signal sequences (antibody signal sequences or signal sequence derived from another protein), one or more furin cleavage sites, one or more linker sequences, one or more antibody light chain variable regions, one or more antibody light chain constant regions, one or more antibody heavy chain variable regions, one or more antibody heavy chain constant regions, a polyadenylation sequence, a stuffer sequence, and/or a filler sequence.

In some embodiments, the AAV viral genome comprises, when read in the 5′ to 3′ direction, a 5′ ITR, a promoter region, an optional intronic region, a signal sequence, an antibody light chain region, a linker region, a signal sequence, an antibody heavy chain region, a polyadenylation sequence, an optional filler sequence, and a 3′ ITR.

The viral genome may encode an antibody fragment, such as, but not limited to Fab, F(ab′)2 or scFv fragments. In some embodiments, the viral genome encodes a Fab antibody fragment. In another embodiment, the viral genome encodes an F(ab′)2 antibody fragment. In some embodiments, the viral genome encodes an scFv.

In some embodiments, the viral genome comprising the ITR to ITR sequence, or a fragment thereof, described in Tables 4-6 and 15-91 is packaged in a capsid having a serotype selected from Table 1 to generate an AAV particle. For example, the capsid serotype is VOY101, VOY201, AAVPHP.B, AAVPHP.N, AAV1, AAV2, AAV2 variant, AAV3, AAV2/3 variant, AAV4, AAV5, AAV6, AAV7, AAV8, AAV9, AAV9.47, AAV9(hu14), AAV9K449R, AAV10, AAV11, AAV12, AAVrh8, AAVrh10, AAVDJ, or AAVDJ8, or any variant thereof. In some embodiments, the capsid serotype is AAVPHP.B, AAV9, AAV6, AAVrh10, and/or AAVDJ.

In some embodiments, the AAV particle comprises a viral genome comprising SEQ ID NO: 1990 and a VOY101 capsid. In some embodiments, the AAV particle comprises a viral genome comprising SEQ ID NO: 1990 and a VOY201 capsid. In some embodiments, the AAV particle comprises a viral genome comprising SEQ ID NO: 1990 and an AAV9 capsid. In some embodiments, the AAV particle comprises a viral genome comprising SEQ ID NO: 1990 and an AAV9K449R capsid. In some embodiments, the AAV particle comprises a viral genome comprising SEQ ID NO: 1990 and an AAVPHP.B capsid. In some embodiments, the AAV particle comprises a viral genome comprising SEQ ID NO: 1990 and an AAVPHP.N capsid. In some embodiments, the AAV particle comprises a viral genome comprising SEQ ID NO: 1990 and an AAV2 variant capsid. In some embodiments, the AAV particle comprises a viral genome comprising SEQ ID NO: 1990 and an AAV2/3 variant capsid. In some embodiments, the AAV particle comprises a viral genome comprising SEQ ID NO: 1990 and a capsid comprising SEQ ID NO: 1. In some embodiments, the AAV particle comprises a viral genome comprising SEQ ID NO: 1990 and a capsid encoded by a nucleic acid sequence comprising SEQ ID NO: 2. In some embodiments, the AAV particle comprises a viral genome comprising SEQ ID NO: 1990 and a capsid comprising SEQ ID NO: 4534. In some embodiments, the AAV particle comprises a viral genome comprising SEQ ID NO: 1990 and a capsid encoded by a nucleic acid sequence comprising SEQ ID NO: 3. In some embodiments, the AAV particle comprises a viral genome comprising SEQ ID NO: 1990 and a capsid comprising SEQ ID NO: 138. In some embodiments, the AAV particle comprises a viral genome comprising SEQ ID NO: 1990 and a capsid encoded by a nucleic acid sequence comprising SEQ ID NO: 137. In some embodiments, the AAV particle comprises a viral genome comprising SEQ ID NO: 1990 and a capsid comprising SEQ ID NO: 5. In some embodiments, the AAV particle comprises a viral genome comprising SEQ ID NO: 1990 and a capsid encoded by a nucleic acid sequence comprising SEQ ID NO: 6. In some embodiments, the AAV particle comprises a viral genome comprising SEQ ID NO: 1990 and a capsid comprising SEQ ID NO: 4. In some embodiments, the AAV particle comprises a viral genome comprising SEQ ID NO: 1990 and a capsid comprising SEQ ID NO: 11. In some embodiments, the AAV particle comprises a viral genome comprising SEQ ID NO: 1990 and a capsid comprising SEQ ID NO: 2679. In some embodiments, the AAV particle comprises a viral genome comprising SEQ ID NO: 1990 and a capsid comprising SEQ ID NO: 2809. In some embodiments, the AAV particle comprises a viral genome comprising SEQ ID NO: 1990 and a capsid comprising SEQ ID NO: 2871.

In some embodiments, the AAV particle comprises a viral genome comprising SEQ ID NO: 1991 and a VOY101 capsid. In some embodiments, the AAV particle comprises a viral genome comprising SEQ ID NO: 1991 and a VOY201 capsid. In some embodiments, the AAV particle comprises a viral genome comprising SEQ ID NO: 1991 and an AAV9 capsid. In some embodiments, the AAV particle comprises a viral genome comprising SEQ ID NO: 1991 and an AAV9K449R capsid. In some embodiments, the AAV particle comprises a viral genome comprising SEQ ID NO: 1991 and an AAVPHP.B capsid. In some embodiments, the AAV particle comprises a viral genome comprising SEQ ID NO: 1991 and an AAVPHP.N capsid. In some embodiments, the AAV particle comprises a viral genome comprising SEQ ID NO: 1991 and an AAV2 variant capsid. In some embodiments, the AAV particle comprises a viral genome comprising SEQ ID NO: 1991 and an AAV2/3 variant capsid. In some embodiments, the AAV particle comprises a viral genome comprising SEQ ID NO: 1991 and a capsid comprising SEQ ID NO: 1. In some embodiments, the AAV particle comprises a viral genome comprising SEQ ID NO: 1991 and a capsid encoded by a nucleic acid sequence comprising SEQ ID NO: 2. In some embodiments, the AAV particle comprises a viral genome comprising SEQ ID NO: 1991 and a capsid comprising SEQ ID NO: 4534. In some embodiments, the AAV particle comprises a viral genome comprising SEQ ID NO: 1991 and a capsid encoded by a nucleic acid sequence comprising SEQ ID NO: 3. In some embodiments, the AAV particle comprises a viral genome comprising SEQ ID NO: 1991 and a capsid comprising SEQ ID NO: 138. In some embodiments, the AAV particle comprises a viral genome comprising SEQ ID NO: 1991 and a capsid encoded by a nucleic acid sequence comprising SEQ ID NO: 137. In some embodiments, the AAV particle comprises a viral genome comprising SEQ ID NO: 1991 and a capsid comprising SEQ ID NO: 5. In some embodiments, the AAV particle comprises a viral genome comprising SEQ ID NO: 1991 and a capsid encoded by a nucleic acid sequence comprising SEQ ID NO: 6. In some embodiments, the AAV particle comprises a viral genome comprising SEQ ID NO: 1991 and a capsid comprising SEQ ID NO: 4. In some embodiments, the AAV particle comprises a viral genome comprising SEQ ID NO: 1991 and a capsid comprising SEQ ID NO: 11. In some embodiments, the AAV particle comprises a viral genome comprising SEQ ID NO: 1991 and a capsid comprising SEQ ID NO: 2679. In some embodiments, the AAV particle comprises a viral genome comprising SEQ ID NO: 1991 and a capsid comprising SEQ ID NO: 2809. In some embodiments, the AAV particle comprises a viral genome comprising SEQ ID NO: 1991 and a capsid comprising SEQ ID NO: 2871.

In some embodiments, the AAV particle comprises a viral genome comprising SEQ ID NO: 1992 and a VOY101 capsid. In some embodiments, the AAV particle comprises a viral genome comprising SEQ ID NO: 1992 and a VOY201 capsid. In some embodiments, the AAV particle comprises a viral genome comprising SEQ ID NO: 1992 and an AAV9 capsid. In some embodiments, the AAV particle comprises a viral genome comprising SEQ ID NO: 1992 and an AAV9K449R capsid. In some embodiments, the AAV particle comprises a viral genome comprising SEQ ID NO: 1992 and an AAVPHP.B capsid. In some embodiments, the AAV particle comprises a viral genome comprising SEQ ID NO: 1992 and an AAVPHP.N capsid. In some embodiments, the AAV particle comprises a viral genome comprising SEQ ID NO: 1992 and an AAV2 variant capsid. In some embodiments, the AAV particle comprises a viral genome comprising SEQ ID NO: 1992 and an AAV2/3 variant capsid. In some embodiments, the AAV particle comprises a viral genome comprising SEQ ID NO: 1992 and a capsid comprising SEQ ID NO: 1. In some embodiments, the AAV particle comprises a viral genome comprising SEQ ID NO: 1992 and a capsid encoded by a nucleic acid sequence comprising SEQ ID NO: 2. In some embodiments, the AAV particle comprises a viral genome comprising SEQ ID NO: 1992 and a capsid comprising SEQ ID NO: 4534. In some embodiments, the AAV particle comprises a viral genome comprising SEQ ID NO: 1992 and a capsid encoded by a nucleic acid sequence comprising SEQ ID NO: 3. In some embodiments, the AAV particle comprises a viral genome comprising SEQ ID NO: 1992 and a capsid comprising SEQ ID NO: 138. In some embodiments, the AAV particle comprises a viral genome comprising SEQ ID NO: 1992 and a capsid encoded by a nucleic acid sequence comprising SEQ ID NO: 137. In some embodiments, the AAV particle comprises a viral genome comprising SEQ ID NO: 1992 and a capsid comprising SEQ ID NO: 5. In some embodiments, the AAV particle comprises a viral genome comprising SEQ ID NO: 1992 and a capsid encoded by a nucleic acid sequence comprising SEQ ID NO: 6. In some embodiments, the AAV particle comprises a viral genome comprising SEQ ID NO: 1992 and a capsid comprising SEQ ID NO: 4. In some embodiments, the AAV particle comprises a viral genome comprising SEQ ID NO: 1992 and a capsid comprising SEQ ID NO: 11. In some embodiments, the AAV particle comprises a viral genome comprising SEQ ID NO: 1992 and a capsid comprising SEQ ID NO: 2679. In some embodiments, the AAV particle comprises a viral genome comprising SEQ ID NO: 1992 and a capsid comprising SEQ ID NO: 2809. In some embodiments, the AAV particle comprises a viral genome comprising SEQ ID NO: 1992 and a capsid comprising SEQ ID NO: 2871.

In some embodiments, the AAV particle comprises a viral genome comprising SEQ ID NO: 1993 and a VOY101 capsid. In some embodiments, the AAV particle comprises a viral genome comprising SEQ ID NO: 1993 and a VOY201 capsid. In some embodiments, the AAV particle comprises a viral genome comprising SEQ ID NO: 1993 and an AAV9 capsid. In some embodiments, the AAV particle comprises a viral genome comprising SEQ ID NO: 1993 and an AAV9K449R capsid. In some embodiments, the AAV particle comprises a viral genome comprising SEQ ID NO: 1993 and an AAVPHP.B capsid. In some embodiments, the AAV particle comprises a viral genome comprising SEQ ID NO: 1993 and an AAVPHP.N capsid. In some embodiments, the AAV particle comprises a viral genome comprising SEQ ID NO: 1993 and an AAV2 variant capsid. In some embodiments, the AAV particle comprises a viral genome comprising SEQ ID NO: 1993 and an AAV2/3 variant capsid. In some embodiments, the AAV particle comprises a viral genome comprising SEQ ID NO: 1993 and a capsid comprising SEQ ID NO: 1. In some embodiments, the AAV particle comprises a viral genome comprising SEQ ID NO: 1993 and a capsid encoded by a nucleic acid sequence comprising SEQ ID NO: 2. In some embodiments, the AAV particle comprises a viral genome comprising SEQ ID NO: 1993 and a capsid comprising SEQ ID NO: 4534. In some embodiments, the AAV particle comprises a viral genome comprising SEQ ID NO: 1993 and a capsid encoded by a nucleic acid sequence comprising SEQ ID NO: 3. In some embodiments, the AAV particle comprises a viral genome comprising SEQ ID NO: 1993 and a capsid comprising SEQ ID NO: 138. In some embodiments, the AAV particle comprises a viral genome comprising SEQ ID NO: 1993 and a capsid encoded by a nucleic acid sequence comprising SEQ ID NO: 137. In some embodiments, the AAV particle comprises a viral genome comprising SEQ ID NO: 1993 and a capsid comprising SEQ ID NO: 5. In some embodiments, the AAV particle comprises a viral genome comprising SEQ ID NO: 1993 and a capsid encoded by a nucleic acid sequence comprising SEQ ID NO: 6. In some embodiments, the AAV particle comprises a viral genome comprising SEQ ID NO: 1993 and a capsid comprising SEQ ID NO: 4. In some embodiments, the AAV particle comprises a viral genome comprising SEQ ID NO: 1993 and a capsid comprising SEQ ID NO: 11. In some embodiments, the AAV particle comprises a viral genome comprising SEQ ID NO: 1993 and a capsid comprising SEQ ID NO: 2679. In some embodiments, the AAV particle comprises a viral genome comprising SEQ ID NO: 1993 and a capsid comprising SEQ ID NO: 2809. In some embodiments, the AAV particle comprises a viral genome comprising SEQ ID NO: 1993 and a capsid comprising SEQ ID NO: 2871.

In some embodiments, the AAV particle comprises a viral genome comprising SEQ ID NO: 1994 and a VOY101 capsid. In some embodiments, the AAV particle comprises a viral genome comprising SEQ ID NO: 1994 and a VOY201 capsid. In some embodiments, the AAV particle comprises a viral genome comprising SEQ ID NO: 1994 and an AAV9 capsid. In some embodiments, the AAV particle comprises a viral genome comprising SEQ ID NO: 1994 and an AAV9K449R capsid. In some embodiments, the AAV particle comprises a viral genome comprising SEQ ID NO: 1994 and an AAVPHP.B capsid. In some embodiments, the AAV particle comprises a viral genome comprising SEQ ID NO: 1994 and an AAVPHP.N capsid. In some embodiments, the AAV particle comprises a viral genome comprising SEQ ID NO: 1994 and an AAV2 variant capsid. In some embodiments, the AAV particle comprises a viral genome comprising SEQ ID NO: 1994 and an AAV2/3 variant capsid. In some embodiments, the AAV particle comprises a viral genome comprising SEQ ID NO: 1994 and a capsid comprising SEQ ID NO: 1. In some embodiments, the AAV particle comprises a viral genome comprising SEQ ID NO: 1994 and a capsid encoded by a nucleic acid sequence comprising SEQ ID NO: 2. In some embodiments, the AAV particle comprises a viral genome comprising SEQ ID NO: 1994 and a capsid comprising SEQ ID NO: 4534. In some embodiments, the AAV particle comprises a viral genome comprising SEQ ID NO: 1994 and a capsid encoded by a nucleic acid sequence comprising SEQ ID NO: 3. In some embodiments, the AAV particle comprises a viral genome comprising SEQ ID NO: 1994 and a capsid comprising SEQ ID NO: 138. In some embodiments, the AAV particle comprises a viral genome comprising SEQ ID NO: 1994 and a capsid encoded by a nucleic acid sequence comprising SEQ ID NO: 137. In some embodiments, the AAV particle comprises a viral genome comprising SEQ ID NO: 1994 and a capsid comprising SEQ ID NO: 5. In some embodiments, the AAV particle comprises a viral genome comprising SEQ ID NO: 1994 and a capsid encoded by a nucleic acid sequence comprising SEQ ID NO: 6. In some embodiments, the AAV particle comprises a viral genome comprising SEQ ID NO: 1994 and a capsid comprising SEQ ID NO: 4. In some embodiments, the AAV particle comprises a viral genome comprising SEQ ID NO: 1994 and a capsid comprising SEQ ID NO: 11. In some embodiments, the AAV particle comprises a viral genome comprising SEQ ID NO: 1994 and a capsid comprising SEQ ID NO: 2679. In some embodiments, the AAV particle comprises a viral genome comprising SEQ ID NO: 1994 and a capsid comprising SEQ ID NO: 2809. In some embodiments, the AAV particle comprises a viral genome comprising SEQ ID NO: 1994 and a capsid comprising SEQ ID NO: 2871.

In some embodiments, the AAV particle comprises a viral genome comprising SEQ ID NO: 1995 and a VOY101 capsid. In some embodiments, the AAV particle comprises a viral genome comprising SEQ ID NO: 1995 and a VOY201 capsid. In some embodiments, the AAV particle comprises a viral genome comprising SEQ ID NO: 1995 and an AAV9 capsid. In some embodiments, the AAV particle comprises a viral genome comprising SEQ ID NO: 1995 and an AAV9K449R capsid. In some embodiments, the AAV particle comprises a viral genome comprising SEQ ID NO: 1995 and an AAVPHP.B capsid. In some embodiments, the AAV particle comprises a viral genome comprising SEQ ID NO: 1995 and an AAVPHP.N capsid. In some embodiments, the AAV particle comprises a viral genome comprising SEQ ID NO: 1995 and an AAV2 variant capsid. In some embodiments, the AAV particle comprises a viral genome comprising SEQ ID NO: 1995 and an AAV2/3 variant capsid. In some embodiments, the AAV particle comprises a viral genome comprising SEQ ID NO: 1995 and a capsid comprising SEQ ID NO: 1. In some embodiments, the AAV particle comprises a viral genome comprising SEQ ID NO: 1995 and a capsid encoded by a nucleic acid sequence comprising SEQ ID NO: 2. In some embodiments, the AAV particle comprises a viral genome comprising SEQ ID NO: 1995 and a capsid comprising SEQ ID NO: 4534. In some embodiments, the AAV particle comprises a viral genome comprising SEQ ID NO: 1995 and a capsid encoded by a nucleic acid sequence comprising SEQ ID NO: 3. In some embodiments, the AAV particle comprises a viral genome comprising SEQ ID NO: 1995 and a capsid comprising SEQ ID NO: 138. In some embodiments, the AAV particle comprises a viral genome comprising SEQ ID NO: 1995 and a capsid encoded by a nucleic acid sequence comprising SEQ ID NO: 137. In some embodiments, the AAV particle comprises a viral genome comprising SEQ ID NO: 1995 and a capsid comprising SEQ ID NO: 5. In some embodiments, the AAV particle comprises a viral genome comprising SEQ ID NO: 1995 and a capsid encoded by a nucleic acid sequence comprising SEQ ID NO: 6. In some embodiments, the AAV particle comprises a viral genome comprising SEQ ID NO: 1995 and a capsid comprising SEQ ID NO: 4. In some embodiments, the AAV particle comprises a viral genome comprising SEQ ID NO: 1995 and a capsid comprising SEQ ID NO: 11. In some embodiments, the AAV particle comprises a viral genome comprising SEQ ID NO: 1995 and a capsid comprising SEQ ID NO: 2679. In some embodiments, the AAV particle comprises a viral genome comprising SEQ ID NO: 1995 and a capsid comprising SEQ ID NO: 2809. In some embodiments, the AAV particle comprises a viral genome comprising SEQ ID NO: 1995 and a capsid comprising SEQ ID NO: 2871.

In some embodiments, the AAV particle comprises a viral genome comprising SEQ ID NO: 1996 and a VOY101 capsid. In some embodiments, the AAV particle comprises a viral genome comprising SEQ ID NO: 1996 and a VOY201 capsid. In some embodiments, the AAV particle comprises a viral genome comprising SEQ ID NO: 1996 and an AAV9 capsid. In some embodiments, the AAV particle comprises a viral genome comprising SEQ ID NO: 1996 and an AAV9K449R capsid. In some embodiments, the AAV particle comprises a viral genome comprising SEQ ID NO: 1996 and an AAVPHP.B capsid. In some embodiments, the AAV particle comprises a viral genome comprising SEQ ID NO: 1996 and an AAVPHP.N capsid. In some embodiments, the AAV particle comprises a viral genome comprising SEQ ID NO: 1996 and an AAV2 variant capsid. In some embodiments, the AAV particle comprises a viral genome comprising SEQ ID NO: 1996 and an AAV2/3 variant capsid. In some embodiments, the AAV particle comprises a viral genome comprising SEQ ID NO: 1996 and a capsid comprising SEQ ID NO: 1. In some embodiments, the AAV particle comprises a viral genome comprising SEQ ID NO: 1996 and a capsid encoded by a nucleic acid sequence comprising SEQ ID NO: 2. In some embodiments, the AAV particle comprises a viral genome comprising SEQ ID NO: 1996 and a capsid comprising SEQ ID NO: 4534. In some embodiments, the AAV particle comprises a viral genome comprising SEQ ID NO: 1996 and a capsid encoded by a nucleic acid sequence comprising SEQ ID NO: 3. In some embodiments, the AAV particle comprises a viral genome comprising SEQ ID NO: 1996 and a capsid comprising SEQ ID NO: 138. In some embodiments, the AAV particle comprises a viral genome comprising SEQ ID NO: 1996 and a capsid encoded by a nucleic acid sequence comprising SEQ ID NO: 137. In some embodiments, the AAV particle comprises a viral genome comprising SEQ ID NO: 1996 and a capsid comprising SEQ ID NO: 5. In some embodiments, the AAV particle comprises a viral genome comprising SEQ ID NO: 1996 and a capsid encoded by a nucleic acid sequence comprising SEQ ID NO: 6. In some embodiments, the AAV particle comprises a viral genome comprising SEQ ID NO: 1996 and a capsid comprising SEQ ID NO: 4. In some embodiments, the AAV particle comprises a viral genome comprising SEQ ID NO: 1996 and a capsid comprising SEQ ID NO: 11. In some embodiments, the AAV particle comprises a viral genome comprising SEQ ID NO: 1996 and a capsid comprising SEQ ID NO: 2679. In some embodiments, the AAV particle comprises a viral genome comprising SEQ ID NO: 1996 and a capsid comprising SEQ ID NO: 2809. In some embodiments, the AAV particle comprises a viral genome comprising SEQ ID NO: 1996 and a capsid comprising SEQ ID NO: 2871.

In some embodiments, the AAV particle comprises a viral genome comprising SEQ ID NO: 1997 and a VOY101 capsid. In some embodiments, the AAV particle comprises a viral genome comprising SEQ ID NO: 1997 and a VOY201 capsid. In some embodiments, the AAV particle comprises a viral genome comprising SEQ ID NO: 1997 and an AAV9 capsid. In some embodiments, the AAV particle comprises a viral genome comprising SEQ ID NO: 1997 and an AAV9K449R capsid. In some embodiments, the AAV particle comprises a viral genome comprising SEQ ID NO: 1997 and an AAVPHP.B capsid. In some embodiments, the AAV particle comprises a viral genome comprising SEQ ID NO: 1997 and an AAVPHP.N capsid. In some embodiments, the AAV particle comprises a viral genome comprising SEQ ID NO: 1997 and an AAV2 variant capsid. In some embodiments, the AAV particle comprises a viral genome comprising SEQ ID NO: 1997 and an AAV2/3 variant capsid. In some embodiments, the AAV particle comprises a viral genome comprising SEQ ID NO: 1997 and a capsid comprising SEQ ID NO: 1. In some embodiments, the AAV particle comprises a viral genome comprising SEQ ID NO: 1997 and a capsid encoded by a nucleic acid sequence comprising SEQ ID NO: 2. In some embodiments, the AAV particle comprises a viral genome comprising SEQ ID NO: 1997 and a capsid comprising SEQ ID NO: 4534. In some embodiments, the AAV particle comprises a viral genome comprising SEQ ID NO: 1997 and a capsid encoded by a nucleic acid sequence comprising SEQ ID NO: 3. In some embodiments, the AAV particle comprises a viral genome comprising SEQ ID NO: 1997 and a capsid comprising SEQ ID NO: 138. In some embodiments, the AAV particle comprises a viral genome comprising SEQ ID NO: 1997 and a capsid encoded by a nucleic acid sequence comprising SEQ ID NO: 137. In some embodiments, the AAV particle comprises a viral genome comprising SEQ ID NO: 1997 and a capsid comprising SEQ ID NO: 5. In some embodiments, the AAV particle comprises a viral genome comprising SEQ ID NO: 1997 and a capsid encoded by a nucleic acid sequence comprising SEQ ID NO: 6. In some embodiments, the AAV particle comprises a viral genome comprising SEQ ID NO: 1997 and a capsid comprising SEQ ID NO: 4. In some embodiments, the AAV particle comprises a viral genome comprising SEQ ID NO: 1997 and a capsid comprising SEQ ID NO: 11. In some embodiments, the AAV particle comprises a viral genome comprising SEQ ID NO: 1997 and a capsid comprising SEQ ID NO: 2679. In some embodiments, the AAV particle comprises a viral genome comprising SEQ ID NO: 1997 and a capsid comprising SEQ ID NO: 2809. In some embodiments, the AAV particle comprises a viral genome comprising SEQ ID NO: 1997 and a capsid comprising SEQ ID NO: 2871.

In some embodiments, the AAV particle comprises a viral genome comprising SEQ ID NO: 1998 and a VOY101 capsid. In some embodiments, the AAV particle comprises a viral genome comprising SEQ ID NO: 1998 and a VOY201 capsid. In some embodiments, the AAV particle comprises a viral genome comprising SEQ ID NO: 1998 and an AAV9 capsid. In some embodiments, the AAV particle comprises a viral genome comprising SEQ ID NO: 1998 and an AAV9K449R capsid. In some embodiments, the AAV particle comprises a viral genome comprising SEQ ID NO: 1998 and an AAVPHP.B capsid. In some embodiments, the AAV particle comprises a viral gnome comprising SEQ ID NO: 1998 and an AAVPHP.N capsid. In some embodiments, the AAV particle comprises a viral gnome comprising SEQ ID NO: 1998 and an AAV2 variant capsid. In some embodiments, the AAV particle comprises a viral genome comprising SEQ ID NO: 1998 and an AAV2/3 variant capsid. In some embodiments, the AAV particle comprises a viral genome comprising SEQ ID NO: 1998 and a capsid comprising SEQ ID NO: 1. In some embodiments, the AAV particle comprises a viral genome comprising SEQ ID NO: 1998 and a capsid encoded by a nucleic acid sequence comprising SEQ ID NO: 2. In some embodiments, the AAV particle comprises a viral genome comprising SEQ ID NO: 1998 and a capsid comprising SEQ ID NO: 4534. In some embodiments, the AAV particle comprises a viral genome comprising SEQ ID NO: 1998 and a capsid encoded by a nucleic acid sequence comprising SEQ ID NO: 3. In some embodiments, the AAV particle comprises a viral gnome comprising SEQ ID NO: 1998 and a capsid comprising SEQ ID NO: 138. In some embodiments, the AAV particle comprises a viral genome comprising SEQ ID NO: 1998 and a capsid encoded by a nucleic acid sequence comprising SEQ ID NO: 137. In some embodiments, the AAV particle comprises a viral genome comprising SEQ ID NO: 1998 and a capsid comprising SEQ ID NO: 5. In some embodiments, the AAV particle comprises a viral genome comprising SEQ ID NO: 1998 and a capsid encoded by a nucleic acid sequence comprising SEQ ID NO: 6. In some embodiments, the AAV particle comprises a viral genome comprising SEQ ID NO: 1998 and a capsid comprising SEQ ID NO: 4. In some embodiments, the AAV particle comprises a viral genome comprising SEQ ID NO: 1998 and a capsid comprising SEQ ID NO: 11. In some embodiments, the AAV particle comprises a viral genome comprising SEQ ID NO: 1998 and a capsid comprising SEQ ID NO: 2679. In some embodiments, the AAV particle comprises a viral genome comprising SEQ ID NO: 1998 and a capsid comprising SEQ ID NO: 2809. In some embodiments, the AAV particle comprises a viral genome comprising SEQ ID NO: 1998 and a capsid comprising SEQ ID NO: 2871.

In some embodiments, the AAV particle comprises a viral genome comprising SEQ ID NO: 1999 and a VOY101 capsid. In some embodiments, the AAV particle comprises a viral genome comprising SEQ ID NO: 1999 and a VOY201 capsid. In some embodiments, the AAV particle comprises a viral genome comprising SEQ ID NO: 1999 and an AAV9 capsid. In some embodiments, the AAV particle comprises a viral genome comprising SEQ ID NO: 1999 and an AAV9K449R capsid. In some embodiments, the AAV particle comprises a viral genome comprising SEQ ID NO: 1999 and an AAVPHP.B capsid. In some embodiments, the AAV particle comprises a viral genome comprising SEQ ID NO: 1999 and an AAVPHP.N capsid. In some embodiments, the AAV particle comprises a viral genome comprising SEQ ID NO: 1999 and an AAV2 variant capsid. In some embodiments, the AAV particle comprises a viral genome comprising SEQ ID NO: 1999 and an AAV2/3 variant capsid. In some embodiments, the AAV particle comprises a viral genome comprising SEQ ID NO: 1999 and a capsid comprising SEQ ID NO: 1. In some embodiments, the AAV particle comprises a viral genome comprising SEQ ID NO: 1999 and a capsid encoded by a nucleic acid sequence comprising SEQ ID NO: 2. In some embodiments, the AAV particle comprises a viral genome comprising SEQ ID NO: 1999 and a capsid comprising SEQ ID NO: 4534. In some embodiments, the AAV particle comprises a viral genome comprising SEQ ID NO: 1999 and a capsid encoded by a nucleic acid sequence comprising SEQ ID NO: 3. In some embodiments, the AAV particle comprises a viral genome comprising SEQ ID NO: 1999 and a capsid comprising SEQ ID NO: 138. In some embodiments, the AAV particle comprises a viral genome comprising SEQ ID NO: 1999 and a capsid encoded by a nucleic acid sequence comprising SEQ ID NO: 137. In some embodiments, the AAV particle comprises a viral genome comprising SEQ ID NO: 1999 and a capsid comprising SEQ ID NO: 5. In some embodiments, the AAV particle comprises a viral genome comprising SEQ ID NO: 1999 and a capsid encoded by a nucleic acid sequence comprising SEQ ID NO: 6. In some embodiments, the AAV particle comprises a viral genome comprising SEQ ID NO: 1999 and a capsid comprising SEQ ID NO: 4. In some embodiments, the AAV particle comprises a viral genome comprising SEQ ID NO: 1999 and a capsid comprising SEQ ID NO: 11. In some embodiments, the AAV particle comprises a viral genome comprising SEQ ID NO: 1999 and a capsid comprising SEQ ID NO: 2679. In some embodiments, the AAV particle comprises a viral genome comprising SEQ ID NO: 1999 and a capsid comprising SEQ ID NO: 2809. In some embodiments, the AAV particle comprises a viral genome comprising SEQ ID NO: 1999 and a capsid comprising SEQ ID NO: 2871.

In some embodiments, the AAV particle comprises a viral genome comprising SEQ ID NO: 2000 and a VOY101 capsid. In some embodiments, the AAV particle comprises a viral genome comprising SEQ ID NO: 2000 and a VOY201 capsid. In some embodiments, the AAV particle comprises a viral genome comprising SEQ ID NO: 2000 and an AAV9 capsid. In some embodiments, the AAV particle comprises a viral genome comprising SEQ ID NO: 2000 and an AAV9K449R capsid. In some embodiments, the AAV particle comprises a viral genome comprising SEQ ID NO: 2000 and an AAVPHP.B capsid. In some embodiments, the AAV particle comprises a viral genome comprising SEQ ID NO: 2000 and an AAVPHP.N capsid. In some embodiments, the AAV particle comprises a viral genome comprising SEQ ID NO: 2000 and an AAV2 variant capsid. In some embodiments, the AAV particle comprises a viral genome comprising SEQ ID NO: 2000 and an AAV2/3 variant capsid. In some embodiments, the AAV particle comprises a viral genome comprising SEQ ID NO: 2000 and a capsid comprising SEQ ID NO: 1. In some embodiments, the AAV particle comprises a viral genome comprising SEQ ID NO: 2000 and a capsid encoded by a nucleic acid sequence comprising SEQ ID NO: 2. In some embodiments, the AAV particle comprises a viral genome comprising SEQ ID NO: 2000 and a capsid comprising SEQ ID NO: 4534. In some embodiments, the AAV particle comprises a viral genome comprising SEQ ID NO: 2000 and a capsid encoded by a nucleic acid sequence comprising SEQ ID NO: 3. In some embodiments, the AAV particle comprises a viral genome comprising SEQ ID NO: 2000 and a capsid comprising SEQ ID NO: 138. In some embodiments, the AAV particle comprises a viral genome comprising SEQ ID NO: 2000 and a capsid encoded by a nucleic acid sequence comprising SEQ ID NO: 137. In some embodiments, the AAV particle comprises a viral genome comprising SEQ ID NO: 2000 and a capsid comprising SEQ ID NO: 5. In some embodiments, the AAV particle comprises a viral genome comprising SEQ ID NO: 2000 and a capsid encoded by a nucleic acid sequence comprising SEQ ID NO: 6. In some embodiments, the AAV particle comprises a viral genome comprising SEQ ID NO: 2000 and a capsid comprising SEQ ID NO: 4. In some embodiments, the AAV particle comprises a viral genome comprising SEQ ID NO: 2000 and a capsid comprising SEQ ID NO: 11. In some embodiments, the AAV particle comprises a viral genome comprising SEQ ID NO: 2000 and a capsid comprising SEQ ID NO: 2679. In some embodiments, the AAV particle comprises a viral genome comprising SEQ ID NO: 2000 and a capsid comprising SEQ ID NO: 2809. In some embodiments, the AAV particle comprises a viral genome comprising SEQ ID NO: 2000 and a capsid comprising SEQ ID NO: 2871.

In some embodiments, the AAV particle comprises a viral genome comprising SEQ ID NO: 2001 and a VOY101 capsid. In some embodiments, the AAV particle comprises a viral genome comprising SEQ ID NO: 2001 and a VOY201 capsid. In some embodiments, the AAV particle comprises a viral genome comprising SEQ ID NO: 2001 and an AAV9 capsid. In some embodiments, the AAV particle comprises a viral genome comprising SEQ ID NO: 2001 and an AAV9K449R capsid. In some embodiments, the AAV particle comprises a viral genome comprising SEQ ID NO: 2001 and an AAVPHP.B capsid. In some embodiments, the AAV particle comprises a viral genome comprising SEQ ID NO: 2001 and an AAVPHP.N capsid. In some embodiments, the AAV particle comprises a viral genome comprising SEQ ID NO: 2001 and an AAV2 variant capsid. In some embodiments, the AAV particle comprises a viral genome comprising SEQ ID NO: 2001 and an AAV2/3 variant capsid. In some embodiments, the AAV particle comprises a viral genome comprising SEQ ID NO: 2001 and a capsid comprising SEQ ID NO: 1. In some embodiments, the AAV particle comprises a viral genome comprising SEQ ID NO: 2001 and a capsid encoded by a nucleic acid sequence comprising SEQ ID NO: 2. In some embodiments, the AAV particle comprises a viral genome comprising SEQ ID NO: 2001 and a capsid comprising SEQ ID NO: 4534. In some embodiments, the AAV particle comprises a viral genome comprising SEQ ID NO: 2001 and a capsid encoded by a nucleic acid sequence comprising SEQ ID NO: 3. In some embodiments, the AAV particle comprises a viral genome comprising SEQ ID NO: 2001 and a capsid comprising SEQ ID NO: 138. In some embodiments, the AAV particle comprises a viral genome comprising SEQ ID NO: 2001 and a capsid encoded by a nucleic acid sequence comprising SEQ ID NO: 137. In some embodiments, the AAV particle comprises a viral genome comprising SEQ ID NO: 2001 and a capsid comprising SEQ ID NO: 5. In some embodiments, the AAV particle comprises a viral genome comprising SEQ ID NO: 2001 and a capsid encoded by a nucleic acid sequence comprising SEQ ID NO: 6. In some embodiments, the AAV particle comprises a viral genome comprising SEQ ID NO: 2001 and a capsid comprising SEQ ID NO: 4. In some embodiments, the AAV particle comprises a viral genome comprising SEQ ID NO: 2001 and a capsid comprising SEQ ID NO: 11. In some embodiments, the AAV particle comprises a viral genome comprising SEQ ID NO: 2001 and a capsid comprising SEQ ID NO: 2679. In some embodiments, the AAV particle comprises a viral genome comprising SEQ ID NO: 2001 and a capsid comprising SEQ ID NO: 2809. In some embodiments, the AAV particle comprises a viral genome comprising SEQ ID NO: 2001 and a capsid comprising SEQ ID NO: 2871.

In some embodiments, the AAV particle comprises a viral genome comprising SEQ ID NO: 2002 and a VOY101 capsid. In some embodiments, the AAV particle comprises a viral genome comprising SEQ ID NO: 2002 and a VOY201 capsid. In some embodiments, the AAV particle comprises a viral genome comprising SEQ ID NO: 2002 and an AAV9 capsid. In some embodiments, the AAV particle comprises a viral genome comprising SEQ ID NO: 2002 and an AAV9K449R capsid. In some embodiments, the AAV particle comprises a viral genome comprising SEQ ID NO: 2002 and an AAVPHP.B capsid. In some embodiments, the AAV particle comprises a viral genome comprising SEQ ID NO: 2002 and an AAVPHP.N capsid. In some embodiments, the AAV particle comprises a viral genome comprising SEQ ID NO: 2002 and an AAV2 variant capsid. In some embodiments, the AAV particle comprises a viral genome comprising SEQ ID NO: 2002 and an AAV2/3 variant capsid. In some embodiments, the AAV particle comprises a viral genome comprising SEQ ID NO: 2002 and a capsid comprising SEQ ID NO: 1. In some embodiments, the AAV particle comprises a viral genome comprising SEQ ID NO: 2002 and a capsid encoded by a nucleic acid sequence comprising SEQ ID NO: 2. In some embodiments, the AAV particle comprises a viral genome comprising SEQ ID NO: 2002 and a capsid comprising SEQ ID NO: 4534. In some embodiments, the AAV particle comprises a viral genome comprising SEQ ID NO: 2002 and a capsid encoded by a nucleic acid sequence comprising SEQ ID NO: 3. In some embodiments, the AAV particle comprises a viral genome comprising SEQ ID NO: 2002 and a capsid comprising SEQ ID NO: 138. In some embodiments, the AAV particle comprises a viral genome comprising SEQ ID NO: 2002 and a capsid encoded by a nucleic acid sequence comprising SEQ ID NO: 137. In some embodiments, the AAV particle comprises a viral genome comprising SEQ ID NO: 2002 and a capsid comprising SEQ ID NO: 5. In some embodiments, the AAV particle comprises a viral genome comprising SEQ ID NO: 2002 and a capsid encoded by a nucleic acid sequence comprising SEQ ID NO: 6. In some embodiments, the AAV particle comprises a viral genome comprising SEQ ID NO: 2002 and a capsid comprising SEQ ID NO: 4. In some embodiments, the AAV particle comprises a viral genome comprising SEQ ID NO: 2002 and a capsid comprising SEQ ID NO: 11. In some embodiments, the AAV particle comprises a viral genome comprising SEQ ID NO: 2002 and a capsid comprising SEQ ID NO: 2679. In some embodiments, the AAV particle comprises a viral genome comprising SEQ ID NO: 2002 and a capsid comprising SEQ ID NO: 2809. In some embodiments, the AAV particle comprises a viral genome comprising SEQ ID NO: 2002 and a capsid comprising SEQ ID NO: 2871.

In some embodiments, the AAV particle comprises a viral genome comprising SEQ ID NO: 2003 and a VOY101 capsid. In some embodiments, the AAV particle comprises a viral genome comprising SEQ ID NO: 2003 and a VOY201 capsid. In some embodiments, the AAV particle comprises a viral genome comprising SEQ ID NO: 2003 and an AAV9 capsid. In some embodiments, the AAV particle comprises a viral genome comprising SEQ ID NO: 2003 and an AAV9K449R capsid. In some embodiments, the AAV particle comprises a viral genome comprising SEQ ID NO: 2003 and an AAVPHP.B capsid. In some embodiments, the AAV particle comprises a viral genome comprising SEQ ID NO: 2003 and an AAVPHP.N capsid. In some embodiments, the AAV particle comprises a viral genome comprising SEQ ID NO: 2003 and an AAV2 variant capsid. In some embodiments, the AAV particle comprises a viral genome comprising SEQ ID NO: 2003 and an AAV2/3 variant capsid. In some embodiments, the AAV particle comprises a viral genome comprising SEQ ID NO: 2003 and a capsid comprising SEQ ID NO: 1. In some embodiments, the AAV particle comprises a viral genome comprising SEQ ID NO: 2003 and a capsid encoded by a nucleic acid sequence comprising SEQ ID NO: 2. In some embodiments, the AAV particle comprises a viral genome comprising SEQ ID NO: 2003 and a capsid comprising SEQ ID NO: 4534. In some embodiments, the AAV particle comprises a viral genome comprising SEQ ID NO: 2003 and a capsid encoded by a nucleic acid sequence comprising SEQ ID NO: 3. In some embodiments, the AAV particle comprises a viral genome comprising SEQ ID NO: 2003 and a capsid comprising SEQ ID NO: 138. In some embodiments, the AAV particle comprises a viral genome comprising SEQ ID NO: 2003 and a capsid encoded by a nucleic acid sequence comprising SEQ ID NO: 137. In some embodiments, the AAV particle comprises a viral genome comprising SEQ ID NO: 2003 and a capsid comprising SEQ ID NO: 5. In some embodiments, the AAV particle comprises a viral genome comprising SEQ ID NO: 2003 and a capsid encoded by a nucleic acid sequence comprising SEQ ID NO: 6. In some embodiments, the AAV particle comprises a viral genome comprising SEQ ID NO: 2003 and a capsid comprising SEQ ID NO: 4. In some embodiments, the AAV particle comprises a viral genome comprising SEQ ID NO: 2003 and a capsid comprising SEQ ID NO: 11. In some embodiments, the AAV particle comprises a viral genome comprising SEQ ID NO: 2003 and a capsid comprising SEQ ID NO: 2679. In some embodiments, the AAV particle comprises a viral genome comprising SEQ ID NO: 2003 and a capsid comprising SEQ ID NO: 2809. In some embodiments, the AAV particle comprises a viral genome comprising SEQ ID NO: 2003 and a capsid comprising SEQ ID NO: 2871.

In some embodiments, the AAV particle comprises a viral genome comprising SEQ ID NO: 2004 and a VOY101 capsid. In some embodiments, the AAV particle comprises a viral genome comprising SEQ ID NO: 2004 and a VOY201 capsid. In some embodiments, the AAV particle comprises a viral genome comprising SEQ ID NO: 2004 and an AAV9 capsid. In some embodiments, the AAV particle comprises a viral genome comprising SEQ ID NO: 2004 and an AAV9K449R capsid. In some embodiments, the AAV particle comprises a viral genome comprising SEQ ID NO: 2004 and an AAVPHP.B capsid. In some embodiments, the AAV particle comprises a viral genome comprising SEQ ID NO: 2004 and an AAVPHP.N capsid. In some embodiments, the AAV particle comprises a viral genome comprising SEQ ID NO: 2004 and an AAV2 variant capsid. In some embodiments, the AAV particle comprises a viral genome comprising SEQ ID NO: 2004 and an AAV2/3 variant capsid. In some embodiments, the AAV particle comprises a viral genome comprising SEQ ID NO: 2004 and a capsid comprising SEQ ID NO: 1. In some embodiments, the AAV particle comprises a viral genome comprising SEQ ID NO: 2004 and a capsid encoded by a nucleic acid sequence comprising SEQ ID NO: 2. In some embodiments, the AAV particle comprises a viral genome comprising SEQ ID NO: 2004 and a capsid comprising SEQ ID NO: 4534. In some embodiments, the AAV particle comprises a viral genome comprising SEQ ID NO: 2004 and a capsid encoded by a nucleic acid sequence comprising SEQ ID NO: 3. In some embodiments, the AAV particle comprises a viral genome comprising SEQ ID NO: 2004 and a capsid comprising SEQ ID NO: 138. In some embodiments, the AAV particle comprises a viral genome comprising SEQ ID NO: 2004 and a capsid encoded by a nucleic acid sequence comprising SEQ ID NO: 137. In some embodiments, the AAV particle comprises a viral genome comprising SEQ ID NO: 2004 and a capsid comprising SEQ ID NO: 5. In some embodiments, the AAV particle comprises a viral genome comprising SEQ ID NO: 2004 and a capsid encoded by a nucleic acid sequence comprising SEQ ID NO: 6. In some embodiments, the AAV particle comprises a viral genome comprising SEQ ID NO: 2004 and a capsid comprising SEQ ID NO: 4. In some embodiments, the AAV particle comprises a viral genome comprising SEQ ID NO: 2004 and a capsid comprising SEQ ID NO: 11. In some embodiments, the AAV particle comprises a viral genome comprising SEQ ID NO: 2004 and a capsid comprising SEQ ID NO: 2679. In some embodiments, the AAV particle comprises a viral genome comprising SEQ ID NO: 2004 and a capsid comprising SEQ ID NO: 2809. In some embodiments, the AAV particle comprises a viral genome comprising SEQ ID NO: 2004 and a capsid comprising SEQ ID NO: 2871.

In some embodiments, the AAV particle comprises a viral genome comprising SEQ ID NO: 2005 and a VOY101 capsid. In some embodiments, the AAV particle comprises a viral genome comprising SEQ ID NO: 2005 and a VOY201 capsid. In some embodiments, the AAV particle comprises a viral genome comprising SEQ ID NO: 2005 and an AAV9 capsid. In some embodiments, the AAV particle comprises a viral genome comprising SEQ ID NO: 2005 and an AAV9K449R capsid. In some embodiments, the AAV particle comprises a viral genome comprising SEQ ID NO: 2005 and an AAVPHP.B capsid. In some embodiments, the AAV particle comprises a viral genome comprising SEQ ID NO: 2005 and an AAVPHP.N capsid. In some embodiments, the AAV particle comprises a viral genome comprising SEQ ID NO: 2005 and an AAV2 variant capsid. In some embodiments, the AAV particle comprises a viral genome comprising SEQ ID NO: 2005 and an AAV2/3 variant capsid. In some embodiments, the AAV particle comprises a viral genome comprising SEQ ID NO: 2005 and a capsid comprising SEQ ID NO: 1. In some embodiments, the AAV particle comprises a viral genome comprising SEQ ID NO: 2005 and a capsid encoded by a nucleic acid sequence comprising SEQ ID NO: 2. In some embodiments, the AAV particle comprises a viral genome comprising SEQ ID NO: 2005 and a capsid comprising SEQ ID NO: 4534. In some embodiments, the AAV particle comprises a viral genome comprising SEQ ID NO: 2005 and a capsid encoded by a nucleic acid sequence comprising SEQ ID NO: 3. In some embodiments, the AAV particle comprises a viral genome comprising SEQ ID NO: 2005 and a capsid comprising SEQ ID NO: 138. In some embodiments, the AAV particle comprises a viral genome comprising SEQ ID NO: 2005 and a capsid encoded by a nucleic acid sequence comprising SEQ ID NO: 137. In some embodiments, the AAV particle comprises a viral genome comprising SEQ ID NO: 2005 and a capsid comprising SEQ ID NO: 5. In some embodiments, the AAV particle comprises a viral genome comprising SEQ ID NO: 2005 and a capsid encoded by a nucleic acid sequence comprising SEQ ID NO: 6. In some embodiments, the AAV particle comprises a viral genome comprising SEQ ID NO: 2005 and a capsid comprising SEQ ID NO: 4. In some embodiments, the AAV particle comprises a viral genome comprising SEQ ID NO: 2005 and a capsid comprising SEQ ID NO: 11. In some embodiments, the AAV particle comprises a viral genome comprising SEQ ID NO: 2005 and a capsid comprising SEQ ID NO: 2679. In some embodiments, the AAV particle comprises a viral genome comprising SEQ ID NO: 2005 and a capsid comprising SEQ ID NO: 2809. In some embodiments, the AAV particle comprises a viral genome comprising SEQ ID NO: 2005 and a capsid comprising SEQ ID NO: 2871.

In some embodiments, the AAV particle comprises a viral genome comprising SEQ ID NO: 2006 and a VOY101 capsid. In some embodiments, the AAV particle comprises a viral genome comprising SEQ ID NO: 2006 and a VOY201 capsid. In some embodiments, the AAV particle comprises a viral genome comprising SEQ ID NO: 2006 and an AAV9 capsid. In some embodiments, the AAV particle comprises a viral genome comprising SEQ ID NO: 2006 and an AAV9K449R capsid. In some embodiments, the AAV particle comprises a viral genome comprising SEQ ID NO: 2006 and an AAVPHP.B capsid. In some embodiments, the AAV particle comprises a viral genome comprising SEQ ID NO: 2006 and an AAVPHP.N capsid. In some embodiments, the AAV particle comprises a viral genome comprising SEQ ID NO: 2006 and an AAV2 variant capsid. In some embodiments, the AAV particle comprises a viral genome comprising SEQ ID NO: 2006 and an AAV2/3 variant capsid. In some embodiments, the AAV particle comprises a viral genome comprising SEQ ID NO: 2006 and a capsid comprising SEQ ID NO: 1. In some embodiments, the AAV particle comprises a viral genome comprising SEQ ID NO: 2006 and a capsid encoded by a nucleic acid sequence comprising SEQ ID NO: 2. In some embodiments, the AAV particle comprises a viral genome comprising SEQ ID NO: 2006 and a capsid comprising SEQ ID NO: 4534. In some embodiments, the AAV particle comprises a viral genome comprising SEQ ID NO: 2006 and a capsid encoded by a nucleic acid sequence comprising SEQ ID NO: 3. In some embodiments, the AAV particle comprises a viral genome comprising SEQ ID NO: 2006 and a capsid comprising SEQ ID NO: 138. In some embodiments, the AAV particle comprises a viral genome comprising SEQ ID NO: 2006 and a capsid encoded by a nucleic acid sequence comprising SEQ ID NO: 137. In some embodiments, the AAV particle comprises a viral genome comprising SEQ ID NO: 2006 and a capsid comprising SEQ ID NO: 5. In some embodiments, the AAV particle comprises a viral genome comprising SEQ ID NO: 2006 and a capsid encoded by a nucleic acid sequence comprising SEQ ID NO: 6. In some embodiments, the AAV particle comprises a viral genome comprising SEQ ID NO: 2006 and a capsid comprising SEQ ID NO: 4. In some embodiments, the AAV particle comprises a viral genome comprising SEQ ID NO: 2006 and a capsid comprising SEQ ID NO: 11. In some embodiments, the AAV particle comprises a viral genome comprising SEQ ID NO: 2006 and a capsid comprising SEQ ID NO: 2679. In some embodiments, the AAV particle comprises a viral genome comprising SEQ ID NO: 2006 and a capsid comprising SEQ ID NO: 2809. In some embodiments, the AAV particle comprises a viral genome comprising SEQ ID NO: 2006 and a capsid comprising SEQ ID NO: 2871.

In some embodiments, the AAV particle comprises a viral genome comprising SEQ ID NO: 2007 and a VOY101 capsid. In some embodiments, the AAV particle comprises a viral genome comprising SEQ ID NO: 2007 and a VOY201 capsid. In some embodiments, the AAV particle comprises a viral genome comprising SEQ ID NO: 2007 and an AAV9 capsid. In some embodiments, the AAV particle comprises a viral genome comprising SEQ ID NO: 2007 and an AAV9K449R capsid. In some embodiments, the AAV particle comprises a viral genome comprising SEQ ID NO: 2007 and an AAVPHP.B capsid. In some embodiments, the AAV particle comprises a viral genome comprising SEQ ID NO: 2007 and an AAVPHP.N capsid. In some embodiments, the AAV particle comprises a viral genome comprising SEQ ID NO: 2007 and an AAV2 variant capsid. In some embodiments, the AAV particle comprises a viral genome comprising SEQ ID NO: 2007 and an AAV2/3 variant capsid. In some embodiments, the AAV particle comprises a viral genome comprising SEQ ID NO: 2007 and a capsid comprising SEQ ID NO: 1. In some embodiments, the AAV particle comprises a viral genome comprising SEQ ID NO: 2007 and a capsid encoded by a nucleic acid sequence comprising SEQ ID NO: 2. In some embodiments, the AAV particle comprises a viral genome comprising SEQ ID NO: 2007 and a capsid comprising SEQ ID NO: 4534. In some embodiments, the AAV particle comprises a viral genome comprising SEQ ID NO: 2007 and a capsid encoded by a nucleic acid sequence comprising SEQ ID NO: 3. In some embodiments, the AAV particle comprises a viral genome comprising SEQ ID NO: 2007 and a capsid comprising SEQ ID NO: 138. In some embodiments, the AAV particle comprises a viral genome comprising SEQ ID NO: 2007 and a capsid encoded by a nucleic acid sequence comprising SEQ ID NO: 137. In some embodiments, the AAV particle comprises a viral genome comprising SEQ ID NO: 2007 and a capsid comprising SEQ ID NO: 5. In some embodiments, the AAV particle comprises a viral genome comprising SEQ ID NO: 2007 and a capsid encoded by a nucleic acid sequence comprising SEQ ID NO: 6. In some embodiments, the AAV particle comprises a viral genome comprising SEQ ID NO: 2007 and a capsid comprising SEQ ID NO: 4. In some embodiments, the AAV particle comprises a viral genome comprising SEQ ID NO: 2007 and a capsid comprising SEQ ID NO: 11. In some embodiments, the AAV particle comprises a viral genome comprising SEQ ID NO: 2007 and a capsid comprising SEQ ID NO: 2679. In some embodiments, the AAV particle comprises a viral genome comprising SEQ ID NO: 2007 and a capsid comprising SEQ ID NO: 2809. In some embodiments, the AAV particle comprises a viral genome comprising SEQ ID NO: 2007 and a capsid comprising SEQ ID NO: 2871.

In some embodiments, the AAV particle comprises a viral genome comprising SEQ ID NO: 2008 and a VOY101 capsid. In some embodiments, the AAV particle comprises a viral genome comprising SEQ ID NO: 2008 and a VOY201 capsid. In some embodiments, the AAV particle comprises a viral genome comprising SEQ ID NO: 2008 and an AAV9 capsid. In some embodiments, the AAV particle comprises a viral genome comprising SEQ ID NO: 2008 and an AAV9K449R capsid. In some embodiments, the AAV particle comprises a viral genome comprising SEQ ID NO: 2008 and an AAVPHP.B capsid. In some embodiments, the AAV particle comprises a viral genome comprising SEQ ID NO: 2008 and an AAVPHP.N capsid. In some embodiments, the AAV particle comprises a viral genome comprising SEQ ID NO: 2008 and an AAV2 variant capsid. In some embodiments, the AAV particle comprises a viral genome comprising SEQ ID NO: 2008 and an AAV2/3 variant capsid. In some embodiments, the AAV particle comprises a viral genome comprising SEQ ID NO: 2008 and a capsid comprising SEQ ID NO: 1. In some embodiments, the AAV particle comprises a viral genome comprising SEQ ID NO: 2008 and a capsid encoded by a nucleic acid sequence comprising SEQ ID NO: 2. In some embodiments, the AAV particle comprises a viral genome comprising SEQ ID NO: 2008 and a capsid comprising SEQ ID NO: 4534. In some embodiments, the AAV particle comprises a viral genome comprising SEQ ID NO: 2008 and a capsid encoded by a nucleic acid sequence comprising SEQ ID NO: 3. In some embodiments, the AAV particle comprises a viral genome comprising SEQ ID NO: 2008 and a capsid comprising SEQ ID NO: 138. In some embodiments, the AAV particle comprises a viral genome comprising SEQ ID NO: 2008 and a capsid encoded by a nucleic acid sequence comprising SEQ ID NO: 137. In some embodiments, the AAV particle comprises a viral genome comprising SEQ ID NO: 2008 and a capsid comprising SEQ ID NO: 5. In some embodiments, the AAV particle comprises a viral genome comprising SEQ ID NO: 2008 and a capsid encoded by a nucleic acid sequence comprising SEQ ID NO: 6. In some embodiments, the AAV particle comprises a viral genome comprising SEQ ID NO: 2008 and a capsid comprising SEQ ID NO: 4. In some embodiments, the AAV particle comprises a viral genome comprising SEQ ID NO: 2008 and a capsid comprising SEQ ID NO: 11. In some embodiments, the AAV particle comprises a viral genome comprising SEQ ID NO: 2008 and a capsid comprising SEQ ID NO: 2679. In some embodiments, the AAV particle comprises a viral genome comprising SEQ ID NO: 2008 and a capsid comprising SEQ ID NO: 2809. In some embodiments, the AAV particle comprises a viral genome comprising SEQ ID NO: 2008 and a capsid comprising SEQ ID NO: 2871.

In some embodiments, the AAV particle comprises a viral genome comprising SEQ ID NO: 2009 and a VOY101 capsid. In some embodiments, the AAV particle comprises a viral genome comprising SEQ ID NO: 2009 and a VOY201 capsid. In some embodiments, the AAV particle comprises a viral genome comprising SEQ ID NO: 2009 and an AAV9 capsid. In some embodiments, the AAV particle comprises a viral genome comprising SEQ ID NO: 2009 and an AAV9K449R capsid. In some embodiments, the AAV particle comprises a viral genome comprising SEQ ID NO: 2009 and an AAVPHP.B capsid. In some embodiments, the AAV particle comprises a viral genome comprising SEQ ID NO: 2009 and an AAVPHP.N capsid. In some embodiments, the AAV particle comprises a viral genome comprising SEQ ID NO: 2009 and an AAV2 variant capsid. In some embodiments, the AAV particle comprises a viral genome comprising SEQ ID NO: 2009 and an AAV2/3 variant capsid. In some embodiments, the AAV particle comprises a viral genome comprising SEQ ID NO: 2009 and a capsid comprising SEQ ID NO: 1. In some embodiments, the AAV particle comprises a viral genome comprising SEQ ID NO: 2009 and a capsid encoded by a nucleic acid sequence comprising SEQ ID NO: 2. In some embodiments, the AAV particle comprises a viral genome comprising SEQ ID NO: 2009 and a capsid comprising SEQ ID NO: 4534. In some embodiments, the AAV particle comprises a viral genome comprising SEQ ID NO: 2009 and a capsid encoded by a nucleic acid sequence comprising SEQ ID NO: 3. In some embodiments, the AAV particle comprises a viral genome comprising SEQ ID NO: 2009 and a capsid comprising SEQ ID NO: 138. In some embodiments, the AAV particle comprises a viral genome comprising SEQ ID NO: 2009 and a capsid encoded by a nucleic acid sequence comprising SEQ ID NO: 137. In some embodiments, the AAV particle comprises a viral genome comprising SEQ ID NO: 2009 and a capsid comprising SEQ ID NO: 5. In some embodiments, the AAV particle comprises a viral genome comprising SEQ ID NO: 2009 and a capsid encoded by a nucleic acid sequence comprising SEQ ID NO: 6. In some embodiments, the AAV particle comprises a viral genome comprising SEQ ID NO: 2009 and a capsid comprising SEQ ID NO: 4. In some embodiments, the AAV particle comprises a viral genome comprising SEQ ID NO: 2009 and a capsid comprising SEQ ID NO: 11. In some embodiments, the AAV particle comprises a viral genome comprising SEQ ID NO: 2009 and a capsid comprising SEQ ID NO: 2679. In some embodiments, the AAV particle comprises a viral genome comprising SEQ ID NO: 2009 and a capsid comprising SEQ ID NO: 2809. In some embodiments, the AAV particle comprises a viral genome comprising SEQ ID NO: 2009 and a capsid comprising SEQ ID NO: 2871.

In some embodiments, the AAV particle comprises a viral genome comprising SEQ ID NO: 2010 and a VOY101 capsid. In some embodiments, the AAV particle comprises a viral genome comprising SEQ ID NO: 2010 and a VOY201 capsid. In some embodiments, the AAV particle comprises a viral genome comprising SEQ ID NO: 2010 and an AAV9 capsid. In some embodiments, the AAV particle comprises a viral genome comprising SEQ ID NO: 2010 and an AAV9K449R capsid. In some embodiments, the AAV particle comprises a viral genome comprising SEQ ID NO: 2010 and an AAVPHP.B capsid. In some embodiments, the AAV particle comprises a viral genome comprising SEQ ID NO: 2010 and an AAVPHP.N capsid. In some embodiments, the AAV particle comprises a viral genome comprising SEQ ID NO: 2010 and an AAV2 variant capsid. In some embodiments, the AAV particle comprises a viral genome comprising SEQ ID NO: 2010 and an AAV2/3 variant capsid. In some embodiments, the AAV particle comprises a viral genome comprising SEQ ID NO: 2010 and a capsid comprising SEQ ID NO: 1. In some embodiments, the AAV particle comprises a viral genome comprising SEQ ID NO: 2010 and a capsid encoded by a nucleic acid sequence comprising SEQ ID NO: 2. In some embodiments, the AAV particle comprises a viral genome comprising SEQ ID NO: 2010 and a capsid comprising SEQ ID NO: 4534. In some embodiments, the AAV particle comprises a viral genome comprising SEQ ID NO: 2010 and a capsid encoded by a nucleic acid sequence comprising SEQ ID NO: 3. In some embodiments, the AAV particle comprises a viral genome comprising SEQ ID NO: 2010 and a capsid comprising SEQ ID NO: 138. In some embodiments, the AAV particle comprises a viral genome comprising SEQ ID NO: 2010 and a capsid encoded by a nucleic acid sequence comprising SEQ ID NO: 137. In some embodiments, the AAV particle comprises a viral genome comprising SEQ ID NO: 2010 and a capsid comprising SEQ ID NO: 5. In some embodiments, the AAV particle comprises a viral genome comprising SEQ ID NO: 2010 and a capsid encoded by a nucleic acid sequence comprising SEQ ID NO: 6. In some embodiments, the AAV particle comprises a viral genome comprising SEQ ID NO: 2010 and a capsid comprising SEQ ID NO: 4. In some embodiments, the AAV particle comprises a viral genome comprising SEQ ID NO: 2010 and a capsid comprising SEQ ID NO: 11. In some embodiments, the AAV particle comprises a viral genome comprising SEQ ID NO: 2010 and a capsid comprising SEQ ID NO: 2679. In some embodiments, the AAV particle comprises a viral genome comprising SEQ ID NO: 2010 and a capsid comprising SEQ ID NO: 2809. In some embodiments, the AAV particle comprises a viral genome comprising SEQ ID NO: 2010 and a capsid comprising SEQ ID NO: 2871.

In some embodiments, the AAV particle comprises a viral genome comprising SEQ ID NO: 2011 and a VOY101 capsid. In some embodiments, the AAV particle comprises a viral genome comprising SEQ ID NO: 2011 and a VOY201 capsid. In some embodiments, the AAV particle comprises a viral genome comprising SEQ ID NO: 2011 and an AAV9 capsid. In some embodiments, the AAV particle comprises a viral genome comprising SEQ ID NO: 2011 and an AAV9K449R capsid. In some embodiments, the AAV particle comprises a viral genome comprising SEQ ID NO: 2011 and an AAVPHP.B capsid. In some embodiments, the AAV particle comprises a viral genome comprising SEQ ID NO: 2011 and an AAVPHP.N capsid. In some embodiments, the AAV particle comprises a viral genome comprising SEQ ID NO: 2011 and an AAV2 variant capsid. In some embodiments, the AAV particle comprises a viral genome comprising SEQ ID NO: 2011 and an AAV2/3 variant capsid. In some embodiments, the AAV particle comprises a viral genome comprising SEQ ID NO: 2011 and a capsid comprising SEQ ID NO: 1. In some embodiments, the AAV particle comprises a viral genome comprising SEQ ID NO: 2011 and a capsid encoded by a nucleic acid sequence comprising SEQ ID NO: 2. In some embodiments, the AAV particle comprises a viral genome comprising SEQ ID NO: 2011 and a capsid comprising SEQ ID NO: 4534. In some embodiments, the AAV particle comprises a viral genome comprising SEQ ID NO: 2011 and a capsid encoded by a nucleic acid sequence comprising SEQ ID NO: 3. In some embodiments, the AAV particle comprises a viral genome comprising SEQ ID NO: 2011 and a capsid comprising SEQ ID NO: 138. In some embodiments, the AAV particle comprises a viral genome comprising SEQ ID NO: 2011 and a capsid encoded by a nucleic acid sequence comprising SEQ ID NO: 137. In some embodiments, the AAV particle comprises a viral genome comprising SEQ ID NO: 2011 and a capsid comprising SEQ ID NO: 5. In some embodiments, the AAV particle comprises a viral genome comprising SEQ ID NO: 2011 and a capsid encoded by a nucleic acid sequence comprising SEQ ID NO: 6. In some embodiments, the AAV particle comprises a viral genome comprising SEQ ID NO: 2011 and a capsid comprising SEQ ID NO: 4. In some embodiments, the AAV particle comprises a viral genome comprising SEQ ID NO: 2011 and a capsid comprising SEQ ID NO: 11. In some embodiments, the AAV particle comprises a viral genome comprising SEQ ID NO: 2011 and a capsid comprising SEQ ID NO: 2679. In some embodiments, the AAV particle comprises a viral genome comprising SEQ ID NO: 2011 and a capsid comprising SEQ ID NO: 2809. In some embodiments, the AAV particle comprises a viral genome comprising SEQ ID NO: 2011 and a capsid comprising SEQ ID NO: 2871.

In some embodiments, the AAV particle comprises a viral genome comprising SEQ ID NO: 2012 and a VOY101 capsid. In some embodiments, the AAV particle comprises a viral genome comprising SEQ ID NO: 2012 and a VOY201 capsid. In some embodiments, the AAV particle comprises a viral genome comprising SEQ ID NO: 2012 and an AAV9 capsid. In some embodiments, the AAV particle comprises a viral genome comprising SEQ ID NO: 2012 and an AAV9K449R capsid. In some embodiments, the AAV particle comprises a viral genome comprising SEQ ID NO: 2012 and an AAVPHP.B capsid. In some embodiments, the AAV particle comprises a viral genome comprising SEQ ID NO: 2012 and an AAVPHP.N capsid. In some embodiments, the AAV particle comprises a viral genome comprising SEQ ID NO: 2012 and an AAV2 variant capsid. In some embodiments, the AAV particle comprises a viral genome comprising SEQ ID NO: 2012 and an AAV2/3 variant capsid. In some embodiments, the AAV particle comprises a viral genome comprising SEQ ID NO: 2012 and a capsid comprising SEQ ID NO: 1. In some embodiments, the AAV particle comprises a viral genome comprising SEQ ID NO: 2012 and a capsid encoded by a nucleic acid sequence comprising SEQ ID NO: 2. In some embodiments, the AAV particle comprises a viral genome comprising SEQ ID NO: 2012 and a capsid comprising SEQ ID NO: 4534. In some embodiments, the AAV particle comprises a viral genome comprising SEQ ID NO: 2012 and a capsid encoded by a nucleic acid sequence comprising SEQ ID NO: 3. In some embodiments, the AAV particle comprises a viral genome comprising SEQ ID NO: 2012 and a capsid comprising SEQ ID NO: 138. In some embodiments, the AAV particle comprises a viral genome comprising SEQ ID NO: 2012 and a capsid encoded by a nucleic acid sequence comprising SEQ ID NO: 137. In some embodiments, the AAV particle comprises a viral genome comprising SEQ ID NO: 2012 and a capsid comprising SEQ ID NO: 5. In some embodiments, the AAV particle comprises a viral genome comprising SEQ ID NO: 2012 and a capsid encoded by a nucleic acid sequence comprising SEQ ID NO: 6. In some embodiments, the AAV particle comprises a viral genome comprising SEQ ID NO: 2012 and a capsid comprising SEQ ID NO: 4. In some embodiments, the AAV particle comprises a viral genome comprising SEQ ID NO: 2012 and a capsid comprising SEQ ID NO: 11. In some embodiments, the AAV particle comprises a viral genome comprising SEQ ID NO: 2012 and a capsid comprising SEQ ID NO: 2679. In some embodiments, the AAV particle comprises a viral genome comprising SEQ ID NO: 2012 and a capsid comprising SEQ ID NO: 2809. In some embodiments, the AAV particle comprises a viral genome comprising SEQ ID NO: 2012 and a capsid comprising SEQ ID NO: 2871.

In some embodiments, the AAV particle comprises a viral genome comprising SEQ ID NO: 2013 and a VOY101 capsid. In some embodiments, the AAV particle comprises a viral genome comprising SEQ ID NO: 2013 and a VOY201 capsid. In some embodiments, the AAV particle comprises a viral genome comprising SEQ ID NO: 2013 and an AAV9 capsid. In some embodiments, the AAV particle comprises a viral genome comprising SEQ ID NO: 2013 and an AAV9K449R capsid. In some embodiments, the AAV particle comprises a viral genome comprising SEQ ID NO: 2013 and an AAVPHP.B capsid. In some embodiments, the AAV particle comprises a viral genome comprising SEQ ID NO: 2013 and an AAVPHP.N capsid. In some embodiments, the AAV particle comprises a viral genome comprising SEQ ID NO: 2013 and an AAV2 variant capsid. In some embodiments, the AAV particle comprises a viral genome comprising SEQ ID NO: 2013 and an AAV2/3 variant capsid. In some embodiments, the AAV particle comprises a viral genome comprising SEQ ID NO: 2013 and a capsid comprising SEQ ID NO: 1. In some embodiments, the AAV particle comprises a viral genome comprising SEQ ID NO: 2013 and a capsid encoded by a nucleic acid sequence comprising SEQ ID NO: 2. In some embodiments, the AAV particle comprises a viral genome comprising SEQ ID NO: 2013 and a capsid comprising SEQ ID NO: 4534. In some embodiments, the AAV particle comprises a viral genome comprising SEQ ID NO: 2013 and a capsid encoded by a nucleic acid sequence comprising SEQ ID NO: 3. In some embodiments, the AAV particle comprises a viral genome comprising SEQ ID NO: 2013 and a capsid comprising SEQ ID NO: 138. In some embodiments, the AAV particle comprises a viral genome comprising SEQ ID NO: 2013 and a capsid encoded by a nucleic acid sequence comprising SEQ ID NO: 137. In some embodiments, the AAV particle comprises a viral genome comprising SEQ ID NO: 2013 and a capsid comprising SEQ ID NO: 5. In some embodiments, the AAV particle comprises a viral genome comprising SEQ ID NO: 2013 and a capsid encoded by a nucleic acid sequence comprising SEQ ID NO: 6. In some embodiments, the AAV particle comprises a viral genome comprising SEQ ID NO: 2013 and a capsid comprising SEQ ID NO: 4. In some embodiments, the AAV particle comprises a viral genome comprising SEQ ID NO: 2013 and a capsid comprising SEQ ID NO: 11. In some embodiments, the AAV particle comprises a viral genome comprising SEQ ID NO: 2013 and a capsid comprising SEQ ID NO: 2679. In some embodiments, the AAV particle comprises a viral genome comprising SEQ ID NO: 2013 and a capsid comprising SEQ ID NO: 2809. In some embodiments, the AAV particle comprises a viral genome comprising SEQ ID NO: 2013 and a capsid comprising SEQ ID NO: 2871.

In some embodiments, the AAV particle comprises a viral genome comprising SEQ ID NO: 2014 and a VOY101 capsid. In some embodiments, the AAV particle comprises a viral genome comprising SEQ ID NO: 2014 and a VOY201 capsid. In some embodiments, the AAV particle comprises a viral genome comprising SEQ ID NO: 2014 and an AAV9 capsid. In some embodiments, the AAV particle comprises a viral genome comprising SEQ ID NO: 2014 and an AAV9K449R capsid. In some embodiments, the AAV particle comprises a viral genome comprising SEQ ID NO: 2014 and an AAVPHP.B capsid. In some embodiments, the AAV particle comprises a viral genome comprising SEQ ID NO: 2014 and an AAVPHP.N capsid. In some embodiments, the AAV particle comprises a viral genome comprising SEQ ID NO: 2014 and an AAV2 variant capsid. In some embodiments, the AAV particle comprises a viral genome comprising SEQ ID NO: 2014 and an AAV2/3 variant capsid. In some embodiments, the AAV particle comprises a viral genome comprising SEQ ID NO: 2014 and a capsid comprising SEQ ID NO: 1. In some embodiments, the AAV particle comprises a viral genome comprising SEQ ID NO: 2014 and a capsid encoded by a nucleic acid sequence comprising SEQ ID NO: 2. In some embodiments, the AAV particle comprises a viral genome comprising SEQ ID NO: 2014 and a capsid comprising SEQ ID NO: 4534. In some embodiments, the AAV particle comprises a viral genome comprising SEQ ID NO: 2014 and a capsid encoded by a nucleic acid sequence comprising SEQ ID NO: 3. In some embodiments, the AAV particle comprises a viral genome comprising SEQ ID NO: 2014 and a capsid comprising SEQ ID NO: 138. In some embodiments, the AAV particle comprises a viral genome comprising SEQ ID NO: 2014 and a capsid encoded by a nucleic acid sequence comprising SEQ ID NO: 137. In some embodiments, the AAV particle comprises a viral genome comprising SEQ ID NO: 2014 and a capsid comprising SEQ ID NO: 5. In some embodiments, the AAV particle comprises a viral genome comprising SEQ ID NO: 2014 and a capsid encoded by a nucleic acid sequence comprising SEQ ID NO: 6. In some embodiments, the AAV particle comprises a viral genome comprising SEQ ID NO: 2014 and a capsid comprising SEQ ID NO: 4. In some embodiments, the AAV particle comprises a viral genome comprising SEQ ID NO: 2014 and a capsid comprising SEQ ID NO: 11. In some embodiments, the AAV particle comprises a viral genome comprising SEQ ID NO: 2014 and a capsid comprising SEQ ID NO: 2679. In some embodiments, the AAV particle comprises a viral genome comprising SEQ ID NO: 2014 and a capsid comprising SEQ ID NO: 2809. In some embodiments, the AAV particle comprises a viral genome comprising SEQ ID NO: 2014 and a capsid comprising SEQ ID NO: 2871.

In some embodiments, the AAV particle comprises a viral genome comprising SEQ ID NO: 2015 and a VOY101 capsid. In some embodiments, the AAV particle comprises a viral genome comprising SEQ ID NO: 2015 and a VOY201 capsid. In some embodiments, the AAV particle comprises a viral genome comprising SEQ ID NO: 2015 and an AAV9 capsid. In some embodiments, the AAV particle comprises a viral genome comprising SEQ ID NO: 2015 and an AAV9K449R capsid. In some embodiments, the AAV particle comprises a viral genome comprising SEQ ID NO: 2015 and an AAVPHP.B capsid. In some embodiments, the AAV particle comprises a viral genome comprising SEQ ID NO: 2015 and an AAVPHP.N capsid. In some embodiments, the AAV particle comprises a viral genome comprising SEQ ID NO: 2015 and an AAV2 variant capsid. In some embodiments, the AAV particle comprises a viral genome comprising SEQ ID NO: 2015 and an AAV2/3 variant capsid. In some embodiments, the AAV particle comprises a viral genome comprising SEQ ID NO: 2015 and a capsid comprising SEQ ID NO: 1. In some embodiments, the AAV particle comprises a viral genome comprising SEQ ID NO: 2015 and a capsid encoded by a nucleic acid sequence comprising SEQ ID NO: 2. In some embodiments, the AAV particle comprises a viral genome comprising SEQ ID NO: 2015 and a capsid comprising SEQ ID NO: 4534. In some embodiments, the AAV particle comprises a viral genome comprising SEQ ID NO: 2015 and a capsid encoded by a nucleic acid sequence comprising SEQ ID NO: 3. In some embodiments, the AAV particle comprises a viral genome comprising SEQ ID NO: 2015 and a capsid comprising SEQ ID NO: 138. In some embodiments, the AAV particle comprises a viral genome comprising SEQ ID NO: 2015 and a capsid encoded by a nucleic acid sequence comprising SEQ ID NO: 137. In some embodiments, the AAV particle comprises a viral genome comprising SEQ ID NO: 2015 and a capsid comprising SEQ ID NO: 5. In some embodiments, the AAV particle comprises a viral genome comprising SEQ ID NO: 2015 and a capsid encoded by a nucleic acid sequence comprising SEQ ID NO: 6. In some embodiments, the AAV particle comprises a viral genome comprising SEQ ID NO: 2015 and a capsid comprising SEQ ID NO: 4. In some embodiments, the AAV particle comprises a viral genome comprising SEQ ID NO: 2015 and a capsid comprising SEQ ID NO: 11. In some embodiments, the AAV particle comprises a viral genome comprising SEQ ID NO: 2015 and a capsid comprising SEQ ID NO: 2679. In some embodiments, the AAV particle comprises a viral genome comprising SEQ ID NO: 2015 and a capsid comprising SEQ ID NO: 2809. In some embodiments, the AAV particle comprises a viral genome comprising SEQ ID NO: 2015 and a capsid comprising SEQ ID NO: 2871.

In some embodiments, the AAV particle comprises a viral genome comprising SEQ ID NO: 2016 and a VOY101 capsid. In some embodiments, the AAV particle comprises a viral genome comprising SEQ ID NO: 2016 and a VOY201 capsid. In some embodiments, the AAV particle comprises a viral genome comprising SEQ ID NO: 2016 and an AAV9 capsid. In some embodiments, the AAV particle comprises a viral genome comprising SEQ ID NO: 2016 and an AAV9K449R capsid. In some embodiments, the AAV particle comprises a viral genome comprising SEQ ID NO: 2016 and an AAVPHP.B capsid. In some embodiments, the AAV particle comprises a viral genome comprising SEQ ID NO: 2016 and an AAVPHP.N capsid. In some embodiments, the AAV particle comprises a viral genome comprising SEQ ID NO: 2016 and an AAV2 variant capsid. In some embodiments, the AAV particle comprises a viral genome comprising SEQ ID NO: 2016 and an AAV2/3 variant capsid. In some embodiments, the AAV particle comprises a viral genome comprising SEQ ID NO: 2016 and a capsid comprising SEQ ID NO: 1. In some embodiments, the AAV particle comprises a viral genome comprising SEQ ID NO: 2016 and a capsid encoded by a nucleic acid sequence comprising SEQ ID NO: 2. In some embodiments, the AAV particle comprises a viral genome comprising SEQ ID NO: 2016 and a capsid comprising SEQ ID NO: 4534. In some embodiments, the AAV particle comprises a viral genome comprising SEQ ID NO: 2016 and a capsid encoded by a nucleic acid sequence comprising SEQ ID NO: 3. In some embodiments, the AAV particle comprises a viral genome comprising SEQ ID NO: 2016 and a capsid comprising SEQ ID NO: 138. In some embodiments, the AAV particle comprises a viral genome comprising SEQ ID NO: 2016 and a capsid encoded by a nucleic acid sequence comprising SEQ ID NO: 137. In some embodiments, the AAV particle comprises a viral genome comprising SEQ ID NO: 2016 and a capsid comprising SEQ ID NO: 5. In some embodiments, the AAV particle comprises a viral genome comprising SEQ ID NO: 2016 and a capsid encoded by a nucleic acid sequence comprising SEQ ID NO: 6. In some embodiments, the AAV particle comprises a viral genome comprising SEQ ID NO: 2016 and a capsid comprising SEQ ID NO: 4. In some embodiments, the AAV particle comprises a viral genome comprising SEQ ID NO: 2016 and a capsid comprising SEQ ID NO: 11. In some embodiments, the AAV particle comprises a viral genome comprising SEQ ID NO: 2016 and a capsid comprising SEQ ID NO: 2679. In some embodiments, the AAV particle comprises a viral genome comprising SEQ ID NO: 2016 and a capsid comprising SEQ ID NO: 2809. In some embodiments, the AAV particle comprises a viral genome comprising SEQ ID NO: 2016 and a capsid comprising SEQ ID NO: 2871.

In some embodiments, the AAV particle comprises a viral genome comprising SEQ ID NO: 2017 and a VOY101 capsid. In some embodiments, the AAV particle comprises a viral genome comprising SEQ ID NO: 2017 and a VOY201 capsid. In some embodiments, the AAV particle comprises a viral genome comprising SEQ ID NO: 2017 and an AAV9 capsid. In some embodiments, the AAV particle comprises a viral genome comprising SEQ ID NO: 2017 and an AAV9K449R capsid. In some embodiments, the AAV particle comprises a viral genome comprising SEQ ID NO: 2017 and an AAVPHP.B capsid. In some embodiments, the AAV particle comprises a viral genome comprising SEQ ID NO: 2017 and an AAVPHP.N capsid. In some embodiments, the AAV particle comprises a viral genome comprising SEQ ID NO: 2017 and an AAV2 variant capsid. In some embodiments, the AAV particle comprises a viral genome comprising SEQ ID NO: 2017 and an AAV2/3 variant capsid. In some embodiments, the AAV particle comprises a viral genome comprising SEQ ID NO: 2017 and a capsid comprising SEQ ID NO: 1. In some embodiments, the AAV particle comprises a viral genome comprising SEQ ID NO: 2017 and a capsid encoded by a nucleic acid sequence comprising SEQ ID NO: 2. In some embodiments, the AAV particle comprises a viral genome comprising SEQ ID NO: 2017 and a capsid comprising SEQ ID NO: 4534. In some embodiments, the AAV particle comprises a viral genome comprising SEQ ID NO: 2017 and a capsid encoded by a nucleic acid sequence comprising SEQ ID NO: 3. In some embodiments, the AAV particle comprises a viral genome comprising SEQ ID NO: 2017 and a capsid comprising SEQ ID NO: 138. In some embodiments, the AAV particle comprises a viral genome comprising SEQ ID NO: 2017 and a capsid encoded by a nucleic acid sequence comprising SEQ ID NO: 137. In some embodiments, the AAV particle comprises a viral genome comprising SEQ ID NO: 2017 and a capsid comprising SEQ ID NO: 5. In some embodiments, the AAV particle comprises a viral genome comprising SEQ ID NO: 2017 and a capsid encoded by a nucleic acid sequence comprising SEQ ID NO: 6. In some embodiments, the AAV particle comprises a viral genome comprising SEQ ID NO: 2017 and a capsid comprising SEQ ID NO: 4. In some embodiments, the AAV particle comprises a viral genome comprising SEQ ID NO: 2017 and a capsid comprising SEQ ID NO: 11. In some embodiments, the AAV particle comprises a viral genome comprising SEQ ID NO: 2017 and a capsid comprising SEQ ID NO: 2679. In some embodiments, the AAV particle comprises a viral genome comprising SEQ ID NO: 2017 and a capsid comprising SEQ ID NO: 2809. In some embodiments, the AAV particle comprises a viral genome comprising SEQ ID NO: 2017 and a capsid comprising SEQ ID NO: 2871.

In some embodiments, the AAV particle comprises a viral genome comprising SEQ ID NO: 2018 and a VOY101 capsid. In some embodiments, the AAV particle comprises a viral genome comprising SEQ ID NO: 2018 and a VOY201 capsid. In some embodiments, the AAV particle comprises a viral genome comprising SEQ ID NO: 2018 and an AAV9 capsid. In some embodiments, the AAV particle comprises a viral genome comprising SEQ ID NO: 2018 and an AAV9K449R capsid. In some embodiments, the AAV particle comprises a viral genome comprising SEQ ID NO: 2018 and an AAVPHP.B capsid. In some embodiments, the AAV particle comprises a viral genome comprising SEQ ID NO: 2018 and an AAVPHP.N capsid. In some embodiments, the AAV particle comprises a viral genome comprising SEQ ID NO: 2018 and an AAV2 variant capsid. In some embodiments, the AAV particle comprises a viral genome comprising SEQ ID NO: 2018 and an AAV2/3 variant capsid. In some embodiments, the AAV particle comprises a viral genome comprising SEQ ID NO: 2018 and a capsid comprising SEQ ID NO: 1. In some embodiments, the AAV particle comprises a viral genome comprising SEQ ID NO: 2018 and a capsid encoded by a nucleic acid sequence comprising SEQ ID NO: 2. In some embodiments, the AAV particle comprises a viral genome comprising SEQ ID NO: 2018 and a capsid comprising SEQ ID NO: 4534. In some embodiments, the AAV particle comprises a viral genome comprising SEQ ID NO: 2018 and a capsid encoded by a nucleic acid sequence comprising SEQ ID NO: 3. In some embodiments, the AAV particle comprises a viral genome comprising SEQ ID NO: 2018 and a capsid comprising SEQ ID NO: 138. In some embodiments, the AAV particle comprises a viral genome comprising SEQ ID NO: 2018 and a capsid encoded by a nucleic acid sequence comprising SEQ ID NO: 137. In some embodiments, the AAV particle comprises a viral genome comprising SEQ ID NO: 2018 and a capsid comprising SEQ ID NO: 5. In some embodiments, the AAV particle comprises a viral genome comprising SEQ ID NO: 2018 and a capsid encoded by a nucleic acid sequence comprising SEQ ID NO: 6. In some embodiments, the AAV particle comprises a viral genome comprising SEQ ID NO: 2018 and a capsid comprising SEQ ID NO: 4. In some embodiments, the AAV particle comprises a viral genome comprising SEQ ID NO: 2018 and a capsid comprising SEQ ID NO: 11. In some embodiments, the AAV particle comprises a viral genome comprising SEQ ID NO: 2018 and a capsid comprising SEQ ID NO: 2679. In some embodiments, the AAV particle comprises a viral genome comprising SEQ ID NO: 2018 and a capsid comprising SEQ ID NO: 2809. In some embodiments, the AAV particle comprises a viral genome comprising SEQ ID NO: 2018 and a capsid comprising SEQ ID NO: 2871.

In some embodiments, the AAV particle comprises a viral genome comprising SEQ ID NO: 2019 and a VOY101 capsid. In some embodiments, the AAV particle comprises a viral genome comprising SEQ ID NO: 2019 and a VOY201 capsid. In some embodiments, the AAV particle comprises a viral genome comprising SEQ ID NO: 2019 and an AAV9 capsid. In some embodiments, the AAV particle comprises a viral genome comprising SEQ ID NO: 2019 and an AAV9K449R capsid. In some embodiments, the AAV particle comprises a viral genome comprising SEQ ID NO: 2019 and an AAVPHP.B capsid. In some embodiments, the AAV particle comprises a viral genome comprising SEQ ID NO: 2019 and an AAVPHP.N capsid. In some embodiments, the AAV particle comprises a viral genome comprising SEQ ID NO: 2019 and an AAV2 variant capsid. In some embodiments, the AAV particle comprises a viral genome comprising SEQ ID NO: 2019 and an AAV2/3 variant capsid. In some embodiments, the AAV particle comprises a viral genome comprising SEQ ID NO: 2019 and a capsid comprising SEQ ID NO: 1. In some embodiments, the AAV particle comprises a viral genome comprising SEQ ID NO: 2019 and a capsid encoded by a nucleic acid sequence comprising SEQ ID NO: 2. In some embodiments, the AAV particle comprises a viral genome comprising SEQ ID NO: 2019 and a capsid comprising SEQ ID NO: 4534. In some embodiments, the AAV particle comprises a viral genome comprising SEQ ID NO: 2019 and a capsid encoded by a nucleic acid sequence comprising SEQ ID NO: 3. In some embodiments, the AAV particle comprises a viral genome comprising SEQ ID NO: 2019 and a capsid comprising SEQ ID NO: 138. In some embodiments, the AAV particle comprises a viral genome comprising SEQ ID NO: 2019 and a capsid encoded by a nucleic acid sequence comprising SEQ ID NO: 137. In some embodiments, the AAV particle comprises a viral genome comprising SEQ ID NO: 2019 and a capsid comprising SEQ ID NO: 5. In some embodiments, the AAV particle comprises a viral genome comprising SEQ ID NO: 2019 and a capsid encoded by a nucleic acid sequence comprising SEQ ID NO: 6. In some embodiments, the AAV particle comprises a viral genome comprising SEQ ID NO: 2019 and a capsid comprising SEQ ID NO: 4. In some embodiments, the AAV particle comprises a viral genome comprising SEQ ID NO: 2019 and a capsid comprising SEQ ID NO: 11. In some embodiments, the AAV particle comprises a viral genome comprising SEQ ID NO: 2019 and a capsid comprising SEQ ID NO: 2679. In some embodiments, the AAV particle comprises a viral genome comprising SEQ ID NO: 2019 and a capsid comprising SEQ ID NO: 2809. In some embodiments, the AAV particle comprises a viral genome comprising SEQ ID NO: 2019 and a capsid comprising SEQ ID NO: 2871.

In some embodiments, the AAV particle comprises a viral genome comprising SEQ ID NO: 2020 and a VOY101 capsid. In some embodiments, the AAV particle comprises a viral genome comprising SEQ ID NO: 2020 and a VOY201 capsid. In some embodiments, the AAV particle comprises a viral genome comprising SEQ ID NO: 2020 and an AAV9 capsid. In some embodiments, the AAV particle comprises a viral genome comprising SEQ ID NO: 2020 and an AAV9K449R capsid. In some embodiments, the AAV particle comprises a viral genome comprising SEQ ID NO: 2020 and an AAVPHP.B capsid. In some embodiments, the AAV particle comprises a viral genome comprising SEQ ID NO: 2020 and an AAVPHP.N capsid. In some embodiments, the AAV particle comprises a viral genome comprising SEQ ID NO: 2020 and an AAV2 variant capsid. In some embodiments, the AAV particle comprises a viral genome comprising SEQ ID NO: 2020 and an AAV2/3 variant capsid. In some embodiments, the AAV particle comprises a viral genome comprising SEQ ID NO: 2020 and a capsid comprising SEQ ID NO: 1. In some embodiments, the AAV particle comprises a viral genome comprising SEQ ID NO: 2020 and a capsid encoded by a nucleic acid sequence comprising SEQ ID NO: 2. In some embodiments, the AAV particle comprises a viral genome comprising SEQ ID NO: 2020 and a capsid comprising SEQ ID NO: 4534. In some embodiments, the AAV particle comprises a viral genome comprising SEQ ID NO: 2020 and a capsid encoded by a nucleic acid sequence comprising SEQ ID NO: 3. In some embodiments, the AAV particle comprises a viral genome comprising SEQ ID NO: 2020 and a capsid comprising SEQ ID NO: 138. In some embodiments, the AAV particle comprises a viral genome comprising SEQ ID NO: 2020 and a capsid encoded by a nucleic acid sequence comprising SEQ ID NO: 137. In some embodiments, the AAV particle comprises a viral genome comprising SEQ ID NO: 2020 and a capsid comprising SEQ ID NO: 5. In some embodiments, the AAV particle comprises a viral genome comprising SEQ ID NO: 2020 and a capsid encoded by a nucleic acid sequence comprising SEQ ID NO: 6. In some embodiments, the AAV particle comprises a viral genome comprising SEQ ID NO: 2020 and a capsid comprising SEQ ID NO: 4. In some embodiments, the AAV particle comprises a viral genome comprising SEQ ID NO: 2020 and a capsid comprising SEQ ID NO: 11. In some embodiments, the AAV particle comprises a viral genome comprising SEQ ID NO: 2020 and a capsid comprising SEQ ID NO: 2679. In some embodiments, the AAV particle comprises a viral genome comprising SEQ ID NO: 2020 and a capsid comprising SEQ ID NO: 2809. In some embodiments, the AAV particle comprises a viral genome comprising SEQ ID NO: 2020 and a capsid comprising SEQ ID NO: 2871.

In some embodiments, the AAV particle comprises a viral genome comprising SEQ ID NO: 2021 and a VOY101 capsid. In some embodiments, the AAV particle comprises a viral genome comprising SEQ ID NO: 2021 and a VOY201 capsid. In some embodiments, the AAV particle comprises a viral genome comprising SEQ ID NO: 2021 and an AAV9 capsid. In some embodiments, the AAV particle comprises a viral genome comprising SEQ ID NO: 2021 and an AAV9K449R capsid. In some embodiments, the AAV particle comprises a viral genome comprising SEQ ID NO: 2021 and an AAVPHP.B capsid. In some embodiments, the AAV particle comprises a viral genome comprising SEQ ID NO: 2021 and an AAVPHP.N capsid. In some embodiments, the AAV particle comprises a viral genome comprising SEQ ID NO: 2021 and an AAV2 variant capsid. In some embodiments, the AAV particle comprises a viral genome comprising SEQ ID NO: 2021 and an AAV2/3 variant capsid. In some embodiments, the AAV particle comprises a viral genome comprising SEQ ID NO: 2021 and a capsid comprising SEQ ID NO: 1. In some embodiments, the AAV particle comprises a viral genome comprising SEQ ID NO: 2021 and a capsid encoded by a nucleic acid sequence comprising SEQ ID NO: 2. In some embodiments, the AAV particle comprises a viral genome comprising SEQ ID NO: 2021 and a capsid comprising SEQ ID NO: 4534. In some embodiments, the AAV particle comprises a viral genome comprising SEQ ID NO: 2021 and a capsid encoded by a nucleic acid sequence comprising SEQ ID NO: 3. In some embodiments, the AAV particle comprises a viral genome comprising SEQ ID NO: 2021 and a capsid comprising SEQ ID NO: 138. In some embodiments, the AAV particle comprises a viral genome comprising SEQ ID NO: 2021 and a capsid encoded by a nucleic acid sequence comprising SEQ ID NO: 137. In some embodiments, the AAV particle comprises a viral genome comprising SEQ ID NO: 2021 and a capsid comprising SEQ ID NO: 5. In some embodiments, the AAV particle comprises a viral genome comprising SEQ ID NO: 2021 and a capsid encoded by a nucleic acid sequence comprising SEQ ID NO: 6. In some embodiments, the AAV particle comprises a viral genome comprising SEQ ID NO: 2021 and a capsid comprising SEQ ID NO: 4. In some embodiments, the AAV particle comprises a viral genome comprising SEQ ID NO: 2021 and a capsid comprising SEQ ID NO: 11. In some embodiments, the AAV particle comprises a viral genome comprising SEQ ID NO: 2021 and a capsid comprising SEQ ID NO: 2679. In some embodiments, the AAV particle comprises a viral genome comprising SEQ ID NO: 2021 and a capsid comprising SEQ ID NO: 2809. In some embodiments, the AAV particle comprises a viral genome comprising SEQ ID NO: 2021 and a capsid comprising SEQ ID NO: 2871.

In some embodiments, the AAV particle comprises a viral genome comprising SEQ ID NO: 2022 and a VOY101 capsid. In some embodiments, the AAV particle comprises a viral genome comprising SEQ ID NO: 2022 and a VOY201 capsid. In some embodiments, the AAV particle comprises a viral genome comprising SEQ ID NO: 2022 and an AAV9 capsid. In some embodiments, the AAV particle comprises a viral genome comprising SEQ ID NO: 2022 and an AAV9K449R capsid. In some embodiments, the AAV particle comprises a viral genome comprising SEQ ID NO: 2022 and an AAVPHP.B capsid. In some embodiments, the AAV particle comprises a viral genome comprising SEQ ID NO: 2022 and an AAVPHP.N capsid. In some embodiments, the AAV particle comprises a viral genome comprising SEQ ID NO: 2022 and an AAV2 variant capsid. In some embodiments, the AAV particle comprises a viral genome comprising SEQ ID NO: 2022 and an AAV2/3 variant capsid. In some embodiments, the AAV particle comprises a viral genome comprising SEQ ID NO: 2022 and a capsid comprising SEQ ID NO: 1. In some embodiments, the AAV particle comprises a viral genome comprising SEQ ID NO: 2022 and a capsid encoded by a nucleic acid sequence comprising SEQ ID NO: 2. In some embodiments, the AAV particle comprises a viral genome comprising SEQ ID NO: 2022 and a capsid comprising SEQ ID NO: 4534. In some embodiments, the AAV particle comprises a viral genome comprising SEQ ID NO: 2022 and a capsid encoded by a nucleic acid sequence comprising SEQ ID NO: 3. In some embodiments, the AAV particle comprises a viral genome comprising SEQ ID NO: 2022 and a capsid comprising SEQ ID NO: 138. In some embodiments, the AAV particle comprises a viral genome comprising SEQ ID NO: 2022 and a capsid encoded by a nucleic acid sequence comprising SEQ ID NO: 137. In some embodiments, the AAV particle comprises a viral genome comprising SEQ ID NO: 2022 and a capsid comprising SEQ ID NO: 5. In some embodiments, the AAV particle comprises a viral genome comprising SEQ ID NO: 2022 and a capsid encoded by a nucleic acid sequence comprising SEQ ID NO: 6. In some embodiments, the AAV particle comprises a viral genome comprising SEQ ID NO: 2022 and a capsid comprising SEQ ID NO: 4. In some embodiments, the AAV particle comprises a viral genome comprising SEQ ID NO: 2022 and a capsid comprising SEQ ID NO: 11. In some embodiments, the AAV particle comprises a viral genome comprising SEQ ID NO: 2022 and a capsid comprising SEQ ID NO: 2679. In some embodiments, the AAV particle comprises a viral genome comprising SEQ ID NO: 2022 and a capsid comprising SEQ ID NO: 2809. In some embodiments, the AAV particle comprises a viral genome comprising SEQ ID NO: 2022 and a capsid comprising SEQ ID NO: 2871.

In some embodiments, the AAV particle comprises a viral genome comprising SEQ ID NO: 2023 and a VOY101 capsid. In some embodiments, the AAV particle comprises a viral genome comprising SEQ ID NO: 2023 and a VOY201 capsid. In some embodiments, the AAV particle comprises a viral genome comprising SEQ ID NO: 2023 and an AAV9 capsid. In some embodiments, the AAV particle comprises a viral genome comprising SEQ ID NO: 2023 and an AAV9K449R capsid. In some embodiments, the AAV particle comprises a viral genome comprising SEQ ID NO: 2023 and an AAVPHP.B capsid. In some embodiments, the AAV particle comprises a viral genome comprising SEQ ID NO: 2023 and an AAVPHP.N capsid. In some embodiments, the AAV particle comprises a viral genome comprising SEQ ID NO: 2023 and an AAV2 variant capsid. In some embodiments, the AAV particle comprises a viral genome comprising SEQ ID NO: 2023 and an AAV2/3 variant capsid. In some embodiments, the AAV particle comprises a viral genome comprising SEQ ID NO: 2023 and a capsid comprising SEQ ID NO: 1. In some embodiments, the AAV particle comprises a viral genome comprising SEQ ID NO: 2023 and a capsid encoded by a nucleic acid sequence comprising SEQ ID NO: 2. In some embodiments, the AAV particle comprises a viral genome comprising SEQ ID NO: 2023 and a capsid comprising SEQ ID NO: 4534. In some embodiments, the AAV particle comprises a viral genome comprising SEQ ID NO: 2023 and a capsid encoded by a nucleic acid sequence comprising SEQ ID NO: 3. In some embodiments, the AAV particle comprises a viral genome comprising SEQ ID NO: 2023 and a capsid comprising SEQ ID NO: 138. In some embodiments, the AAV particle comprises a viral genome comprising SEQ ID NO: 2023 and a capsid encoded by a nucleic acid sequence comprising SEQ ID NO: 137. In some embodiments, the AAV particle comprises a viral genome comprising SEQ ID NO: 2023 and a capsid comprising SEQ ID NO: 5. In some embodiments, the AAV particle comprises a viral genome comprising SEQ ID NO: 2023 and a capsid encoded by a nucleic acid sequence comprising SEQ ID NO: 6. In some embodiments, the AAV particle comprises a viral genome comprising SEQ ID NO: 2023 and a capsid comprising SEQ ID NO: 4. In some embodiments, the AAV particle comprises a viral genome comprising SEQ ID NO: 2023 and a capsid comprising SEQ ID NO: 11. In some embodiments, the AAV particle comprises a viral genome comprising SEQ ID NO: 2023 and a capsid comprising SEQ ID NO: 2679. In some embodiments, the AAV particle comprises a viral genome comprising SEQ ID NO: 2023 and a capsid comprising SEQ ID NO: 2809. In some embodiments, the AAV particle comprises a viral genome comprising SEQ ID NO: 2023 and a capsid comprising SEQ ID NO: 2871.

In some embodiments, the AAV particle comprises a viral genome comprising SEQ ID NO: 2024 and a VOY101 capsid. In some embodiments, the AAV particle comprises a viral genome comprising SEQ ID NO: 2024 and a VOY201 capsid. In some embodiments, the AAV particle comprises a viral genome comprising SEQ ID NO: 2024 and an AAV9 capsid. In some embodiments, the AAV particle comprises a viral genome comprising SEQ ID NO: 2024 and an AAV9K449R capsid. In some embodiments, the AAV particle comprises a viral genome comprising SEQ ID NO: 2024 and an AAVPHP.B capsid. In some embodiments, the AAV particle comprises a viral genome comprising SEQ ID NO: 2024 and an AAVPHP.N capsid. In some embodiments, the AAV particle comprises a viral genome comprising SEQ ID NO: 2024 and an AAV2 variant capsid. In some embodiments, the AAV particle comprises a viral genome comprising SEQ ID NO: 2024 and an AAV2/3 variant capsid. In some embodiments, the AAV particle comprises a viral genome comprising SEQ ID NO: 2024 and a capsid comprising SEQ ID NO: 1. In some embodiments, the AAV particle comprises a viral genome comprising SEQ ID NO: 2024 and a capsid encoded by a nucleic acid sequence comprising SEQ ID NO: 2. In some embodiments, the AAV particle comprises a viral genome comprising SEQ ID NO: 2024 and a capsid comprising SEQ ID NO: 4534. In some embodiments, the AAV particle comprises a viral genome comprising SEQ ID NO: 2024 and a capsid encoded by a nucleic acid sequence comprising SEQ ID NO: 3. In some embodiments, the AAV particle comprises a viral genome comprising SEQ ID NO: 2024 and a capsid comprising SEQ ID NO: 138. In some embodiments, the AAV particle comprises a viral genome comprising SEQ ID NO: 2024 and a capsid encoded by a nucleic acid sequence comprising SEQ ID NO: 137. In some embodiments, the AAV particle comprises a viral genome comprising SEQ ID NO: 2024 and a capsid comprising SEQ ID NO: 5. In some embodiments, the AAV particle comprises a viral genome comprising SEQ ID NO: 2024 and a capsid encoded by a nucleic acid sequence comprising SEQ ID NO: 6. In some embodiments, the AAV particle comprises a viral genome comprising SEQ ID NO: 2024 and a capsid comprising SEQ ID NO: 4. In some embodiments, the AAV particle comprises a viral genome comprising SEQ ID NO: 2024 and a capsid comprising SEQ ID NO: 11. In some embodiments, the AAV particle comprises a viral genome comprising SEQ ID NO: 2024 and a capsid comprising SEQ ID NO: 2679. In some embodiments, the AAV particle comprises a viral genome comprising SEQ ID NO: 2024 and a capsid comprising SEQ ID NO: 2809. In some embodiments, the AAV particle comprises a viral genome comprising SEQ ID NO: 2024 and a capsid comprising SEQ ID NO: 2871.

In some embodiments, the AAV particle comprises a viral genome comprising SEQ ID NO: 2025 and a VOY101 capsid. In some embodiments, the AAV particle comprises a viral genome comprising SEQ ID NO: 2025 and a VOY201 capsid. In some embodiments, the AAV particle comprises a viral genome comprising SEQ ID NO: 2025 and an AAV9 capsid. In some embodiments, the AAV particle comprises a viral genome comprising SEQ ID NO: 2025 and an AAV9K449R capsid. In some embodiments, the AAV particle comprises a viral genome comprising SEQ ID NO: 2025 and an AAVPHP.B capsid. In some embodiments, the AAV particle comprises a viral genome comprising SEQ ID NO: 2025 and an AAVPHP.N capsid. In some embodiments, the AAV particle comprises a viral genome comprising SEQ ID NO: 2025 and an AAV2 variant capsid. In some embodiments, the AAV particle comprises a viral genome comprising SEQ ID NO: 2025 and an AAV2/3 variant capsid. In some embodiments, the AAV particle comprises a viral genome comprising SEQ ID NO: 2025 and a capsid comprising SEQ ID NO: 1. In some embodiments, the AAV particle comprises a viral genome comprising SEQ ID NO: 2025 and a capsid encoded by a nucleic acid sequence comprising SEQ ID NO: 2. In some embodiments, the AAV particle comprises a viral genome comprising SEQ ID NO: 2025 and a capsid comprising SEQ ID NO: 4534. In some embodiments, the AAV particle comprises a viral genome comprising SEQ ID NO: 2025 and a capsid encoded by a nucleic acid sequence comprising SEQ ID NO: 3. In some embodiments, the AAV particle comprises a viral genome comprising SEQ ID NO: 2025 and a capsid comprising SEQ ID NO: 138. In some embodiments, the AAV particle comprises a viral genome comprising SEQ ID NO: 2025 and a capsid encoded by a nucleic acid sequence comprising SEQ ID NO: 137. In some embodiments, the AAV particle comprises a viral genome comprising SEQ ID NO: 2025 and a capsid comprising SEQ ID NO: 5. In some embodiments, the AAV particle comprises a viral genome comprising SEQ ID NO: 2025 and a capsid encoded by a nucleic acid sequence comprising SEQ ID NO: 6. In some embodiments, the AAV particle comprises a viral genome comprising SEQ ID NO: 2025 and a capsid comprising SEQ ID NO: 4. In some embodiments, the AAV particle comprises a viral genome comprising SEQ ID NO: 2025 and a capsid comprising SEQ ID NO: 11. In some embodiments, the AAV particle comprises a viral genome comprising SEQ ID NO: 2025 and a capsid comprising SEQ ID NO: 2679. In some embodiments, the AAV particle comprises a viral genome comprising SEQ ID NO: 2025 and a capsid comprising SEQ ID NO: 2809. In some embodiments, the AAV particle comprises a viral genome comprising SEQ ID NO: 2025 and a capsid comprising SEQ ID NO: 2871.

In some embodiments, the AAV particle comprises a viral genome comprising SEQ ID NO: 2026 and a VOY101 capsid. In some embodiments, the AAV particle comprises a viral genome comprising SEQ ID NO: 2026 and a VOY201 capsid. In some embodiments, the AAV particle comprises a viral genome comprising SEQ ID NO: 2026 and an AAV9 capsid. In some embodiments, the AAV particle comprises a viral genome comprising SEQ ID NO: 2026 and an AAV9K449R capsid. In some embodiments, the AAV particle comprises a viral genome comprising SEQ ID NO: 2026 and an AAVPHP.B capsid. In some embodiments, the AAV particle comprises a viral genome comprising SEQ ID NO: 2026 and an AAVPHP.N capsid. In some embodiments, the AAV particle comprises a viral genome comprising SEQ ID NO: 2026 and an AAV2 variant capsid. In some embodiments, the AAV particle comprises a viral genome comprising SEQ ID NO: 2026 and an AAV2/3 variant capsid. In some embodiments, the AAV particle comprises a viral genome comprising SEQ ID NO: 2026 and a capsid comprising SEQ ID NO: 1. In some embodiments, the AAV particle comprises a viral genome comprising SEQ ID NO: 2026 and a capsid encoded by a nucleic acid sequence comprising SEQ ID NO: 2. In some embodiments, the AAV particle comprises a viral genome comprising SEQ ID NO: 2026 and a capsid comprising SEQ ID NO: 4534. In some embodiments, the AAV particle comprises a viral genome comprising SEQ ID NO: 2026 and a capsid encoded by a nucleic acid sequence comprising SEQ ID NO: 3. In some embodiments, the AAV particle comprises a viral genome comprising SEQ ID NO: 2026 and a capsid comprising SEQ ID NO: 138. In some embodiments, the AAV particle comprises a viral genome comprising SEQ ID NO: 2026 and a capsid encoded by a nucleic acid sequence comprising SEQ ID NO: 137. In some embodiments, the AAV particle comprises a viral genome comprising SEQ ID NO: 2026 and a capsid comprising SEQ ID NO: 5. In some embodiments, the AAV particle comprises a viral genome comprising SEQ ID NO: 2026 and a capsid encoded by a nucleic acid sequence comprising SEQ ID NO: 6. In some embodiments, the AAV particle comprises a viral genome comprising SEQ ID NO: 2026 and a capsid comprising SEQ ID NO: 4. In some embodiments, the AAV particle comprises a viral genome comprising SEQ ID NO: 2026 and a capsid comprising SEQ ID NO: 11. In some embodiments, the AAV particle comprises a viral genome comprising SEQ ID NO: 2026 and a capsid comprising SEQ ID NO: 2679. In some embodiments, the AAV particle comprises a viral genome comprising SEQ ID NO: 2026 and a capsid comprising SEQ ID NO: 2809. In some embodiments, the AAV particle comprises a viral genome comprising SEQ ID NO: 2026 and a capsid comprising SEQ ID NO: 2871.

In some embodiments, the AAV particle comprises a viral genome comprising SEQ ID NO: 2027 and a VOY101 capsid. In some embodiments, the AAV particle comprises a viral genome comprising SEQ ID NO: 2027 and a VOY201 capsid. In some embodiments, the AAV particle comprises a viral genome comprising SEQ ID NO: 2027 and an AAV9 capsid. In some embodiments, the AAV particle comprises a viral genome comprising SEQ ID NO: 2027 and an AAV9K449R capsid. In some embodiments, the AAV particle comprises a viral genome comprising SEQ ID NO: 2027 and an AAVPHP.B capsid. In some embodiments, the AAV particle comprises a viral genome comprising SEQ ID NO: 2027 and an AAVPHP.N capsid. In some embodiments, the AAV particle comprises a viral genome comprising SEQ ID NO: 2027 and an AAV2 variant capsid. In some embodiments, the AAV particle comprises a viral genome comprising SEQ ID NO: 2027 and an AAV2/3 variant capsid. In some embodiments, the AAV particle comprises a viral genome comprising SEQ ID NO: 2027 and a capsid comprising SEQ ID NO: 1. In some embodiments, the AAV particle comprises a viral genome comprising SEQ ID NO: 2027 and a capsid encoded by a nucleic acid sequence comprising SEQ ID NO: 2. In some embodiments, the AAV particle comprises a viral genome comprising SEQ ID NO: 2027 and a capsid comprising SEQ ID NO: 4534. In some embodiments, the AAV particle comprises a viral genome comprising SEQ ID NO: 2027 and a capsid encoded by a nucleic acid sequence comprising SEQ ID NO: 3. In some embodiments, the AAV particle comprises a viral genome comprising SEQ ID NO: 2027 and a capsid comprising SEQ ID NO: 138. In some embodiments, the AAV particle comprises a viral genome comprising SEQ ID NO: 2027 and a capsid encoded by a nucleic acid sequence comprising SEQ ID NO: 137. In some embodiments, the AAV particle comprises a viral genome comprising SEQ ID NO: 2027 and a capsid comprising SEQ ID NO: 5. In some embodiments, the AAV particle comprises a viral genome comprising SEQ ID NO: 2027 and a capsid encoded by a nucleic acid sequence comprising SEQ ID NO: 6. In some embodiments, the AAV particle comprises a viral genome comprising SEQ ID NO: 2027 and a capsid comprising SEQ ID NO: 4. In some embodiments, the AAV particle comprises a viral genome comprising SEQ ID NO: 2027 and a capsid comprising SEQ ID NO: 11. In some embodiments, the AAV particle comprises a viral genome comprising SEQ ID NO: 2027 and a capsid comprising SEQ ID NO: 2679. In some embodiments, the AAV particle comprises a viral genome comprising SEQ ID NO: 2027 and a capsid comprising SEQ ID NO: 2809. In some embodiments, the AAV particle comprises a viral genome comprising SEQ ID NO: 2027 and a capsid comprising SEQ ID NO: 2871.

In some embodiments, the AAV particle comprises a viral genome comprising SEQ ID NO: 2028 and a VOY101 capsid. In some embodiments, the AAV particle comprises a viral genome comprising SEQ ID NO: 2028 and a VOY201 capsid. In some embodiments, the AAV particle comprises a viral genome comprising SEQ ID NO: 2028 and an AAV9 capsid. In some embodiments, the AAV particle comprises a viral genome comprising SEQ ID NO: 2028 and an AAV9K449R capsid. In some embodiments, the AAV particle comprises a viral genome comprising SEQ ID NO: 2028 and an AAVPHP.B capsid. In some embodiments, the AAV particle comprises a viral genome comprising SEQ ID NO: 2028 and an AAVPHP.N capsid. In some embodiments, the AAV particle comprises a viral genome comprising SEQ ID NO: 2028 and an AAV2 variant capsid. In some embodiments, the AAV particle comprises a viral genome comprising SEQ ID NO: 2028 and an AAV2/3 variant capsid. In some embodiments, the AAV particle comprises a viral genome comprising SEQ ID NO: 2028 and a capsid comprising SEQ ID NO: 1. In some embodiments, the AAV particle comprises a viral genome comprising SEQ ID NO: 2028 and a capsid encoded by a nucleic acid sequence comprising SEQ ID NO: 2. In some embodiments, the AAV particle comprises a viral genome comprising SEQ ID NO: 2028 and a capsid comprising SEQ ID NO: 4534. In some embodiments, the AAV particle comprises a viral genome comprising SEQ ID NO: 2028 and a capsid encoded by a nucleic acid sequence comprising SEQ ID NO: 3. In some embodiments, the AAV particle comprises a viral genome comprising SEQ ID NO: 2028 and a capsid comprising SEQ ID NO: 138. In some embodiments, the AAV particle comprises a viral genome comprising SEQ ID NO: 2028 and a capsid encoded by a nucleic acid sequence comprising SEQ ID NO: 137. In some embodiments, the AAV particle comprises a viral genome comprising SEQ ID NO: 2028 and a capsid comprising SEQ ID NO: 5. In some embodiments, the AAV particle comprises a viral genome comprising SEQ ID NO: 2028 and a capsid encoded by a nucleic acid sequence comprising SEQ ID NO: 6. In some embodiments, the AAV particle comprises a viral genome comprising SEQ ID NO: 2028 and a capsid comprising SEQ ID NO: 4. In some embodiments, the AAV particle comprises a viral genome comprising SEQ ID NO: 2028 and a capsid comprising SEQ ID NO: 11. In some embodiments, the AAV particle comprises a viral genome comprising SEQ ID NO: 2028 and a capsid comprising SEQ ID NO: 2679. In some embodiments, the AAV particle comprises a viral genome comprising SEQ ID NO: 2028 and a capsid comprising SEQ ID NO: 2809. In some embodiments, the AAV particle comprises a viral genome comprising SEQ ID NO: 2028 and a capsid comprising SEQ ID NO: 2871.

In some embodiments, the AAV particle comprises a viral genome comprising SEQ ID NO: 2029 and a VOY101 capsid. In some embodiments, the AAV particle comprises a viral genome comprising SEQ ID NO: 2029 and a VOY201 capsid. In some embodiments, the AAV particle comprises a viral genome comprising SEQ ID NO: 2029 and an AAV9 capsid. In some embodiments, the AAV particle comprises a viral genome comprising SEQ ID NO: 2029 and an AAV9K449R capsid. In some embodiments, the AAV particle comprises a viral genome comprising SEQ ID NO: 2029 and an AAVPHP.B capsid. In some embodiments, the AAV particle comprises a viral genome comprising SEQ ID NO: 2029 and an AAVPHP.N capsid. In some embodiments, the AAV particle comprises a viral genome comprising SEQ ID NO: 2029 and an AAV2 variant capsid. In some embodiments, the AAV particle comprises a viral genome comprising SEQ ID NO: 2029 and an AAV2/3 variant capsid. In some embodiments, the AAV particle comprises a viral genome comprising SEQ ID NO: 2029 and a capsid comprising SEQ ID NO: 1. In some embodiments, the AAV particle comprises a viral genome comprising SEQ ID NO: 2029 and a capsid encoded by a nucleic acid sequence comprising SEQ ID NO: 2. In some embodiments, the AAV particle comprises a viral genome comprising SEQ ID NO: 2029 and a capsid comprising SEQ ID NO: 4534. In some embodiments, the AAV particle comprises a viral genome comprising SEQ ID NO: 2029 and a capsid encoded by a nucleic acid sequence comprising SEQ ID NO: 3. In some embodiments, the AAV particle comprises a viral genome comprising SEQ ID NO: 2029 and a capsid comprising SEQ ID NO: 138. In some embodiments, the AAV particle comprises a viral genome comprising SEQ ID NO: 2029 and a capsid encoded by a nucleic acid sequence comprising SEQ ID NO: 137. In some embodiments, the AAV particle comprises a viral genome comprising SEQ ID NO: 2029 and a capsid comprising SEQ ID NO: 5. In some embodiments, the AAV particle comprises a viral genome comprising SEQ ID NO: 2029 and a capsid encoded by a nucleic acid sequence comprising SEQ ID NO: 6. In some embodiments, the AAV particle comprises a viral genome comprising SEQ ID NO: 2029 and a capsid comprising SEQ ID NO: 4. In some embodiments, the AAV particle comprises a viral genome comprising SEQ ID NO: 2029 and a capsid comprising SEQ ID NO: 11. In some embodiments, the AAV particle comprises a viral genome comprising SEQ ID NO: 2029 and a capsid comprising SEQ ID NO: 2679. In some embodiments, the AAV particle comprises a viral genome comprising SEQ ID NO: 2029 and a capsid comprising SEQ ID NO: 2809. In some embodiments, the AAV particle comprises a viral genome comprising SEQ ID NO: 2029 and a capsid comprising SEQ ID NO: 2871.

In some embodiments, the AAV particle comprises a viral genome comprising SEQ ID NO: 2030 and a VOY101 capsid. In some embodiments, the AAV particle comprises a viral genome comprising SEQ ID NO: 2030 and a VOY201 capsid. In some embodiments, the AAV particle comprises a viral genome comprising SEQ ID NO: 2030 and an AAV9 capsid. In some embodiments, the AAV particle comprises a viral genome comprising SEQ ID NO: 2030 and an AAV9K449R capsid. In some embodiments, the AAV particle comprises a viral genome comprising SEQ ID NO: 2030 and an AAVPHP.B capsid. In some embodiments, the AAV particle comprises a viral genome comprising SEQ ID NO: 2030 and an AAVPHP.N capsid. In some embodiments, the AAV particle comprises a viral genome comprising SEQ ID NO: 2030 and an AAV2 variant capsid. In some embodiments, the AAV particle comprises a viral genome comprising SEQ ID NO: 2030 and an AAV2/3 variant capsid. In some embodiments, the AAV particle comprises a viral genome comprising SEQ ID NO: 2030 and a capsid comprising SEQ ID NO: 1. In some embodiments, the AAV particle comprises a viral genome comprising SEQ ID NO: 2030 and a capsid encoded by a nucleic acid sequence comprising SEQ ID NO: 2. In some embodiments, the AAV particle comprises a viral genome comprising SEQ ID NO: 2030 and a capsid comprising SEQ ID NO: 4534. In some embodiments, the AAV particle comprises a viral genome comprising SEQ ID NO: 2030 and a capsid encoded by a nucleic acid sequence comprising SEQ ID NO: 3. In some embodiments, the AAV particle comprises a viral genome comprising SEQ ID NO: 2030 and a capsid comprising SEQ ID NO: 138. In some embodiments, the AAV particle comprises a viral genome comprising SEQ ID NO: 2030 and a capsid encoded by a nucleic acid sequence comprising SEQ ID NO: 137. In some embodiments, the AAV particle comprises a viral genome comprising SEQ ID NO: 2030 and a capsid comprising SEQ ID NO: 5. In some embodiments, the AAV particle comprises a viral genome comprising SEQ ID NO: 2030 and a capsid encoded by a nucleic acid sequence comprising SEQ ID NO: 6. In some embodiments, the AAV particle comprises a viral genome comprising SEQ ID NO: 2030 and a capsid comprising SEQ ID NO: 4. In some embodiments, the AAV particle comprises a viral genome comprising SEQ ID NO: 2030 and a capsid comprising SEQ ID NO: 11. In some embodiments, the AAV particle comprises a viral genome comprising SEQ ID NO: 2030 and a capsid comprising SEQ ID NO: 2679. In some embodiments, the AAV particle comprises a viral genome comprising SEQ ID NO: 2030 and a capsid comprising SEQ ID NO: 2809. In some embodiments, the AAV particle comprises a viral genome comprising SEQ ID NO: 2030 and a capsid comprising SEQ ID NO: 2871.

In some embodiments, the AAV particle comprises a viral genome comprising SEQ ID NO: 2031 and a VOY101 capsid. In some embodiments, the AAV particle comprises a viral genome comprising SEQ ID NO: 2031 and a VOY201 capsid. In some embodiments, the AAV particle comprises a viral genome comprising SEQ ID NO: 2031 and an AAV9 capsid. In some embodiments, the AAV particle comprises a viral genome comprising SEQ ID NO: 2031 and an AAV9K449R capsid. In some embodiments, the AAV particle comprises a viral genome comprising SEQ ID NO: 2031 and an AAVPHP.B capsid. In some embodiments, the AAV particle comprises a viral genome comprising SEQ ID NO: 2031 and an AAVPHP.N capsid. In some embodiments, the AAV particle comprises a viral genome comprising SEQ ID NO: 2031 and an AAV2 variant capsid. In some embodiments, the AAV particle comprises a viral genome comprising SEQ ID NO: 2031 and an AAV2/3 variant capsid. In some embodiments, the AAV particle comprises a viral genome comprising SEQ ID NO: 2031 and a capsid comprising SEQ ID NO: 1. In some embodiments, the AAV particle comprises a viral genome comprising SEQ ID NO: 2031 and a capsid encoded by a nucleic acid sequence comprising SEQ ID NO: 2. In some embodiments, the AAV particle comprises a viral genome comprising SEQ ID NO: 2031 and a capsid comprising SEQ ID NO: 4534. In some embodiments, the AAV particle comprises a viral genome comprising SEQ ID NO: 2031 and a capsid encoded by a nucleic acid sequence comprising SEQ ID NO: 3. In some embodiments, the AAV particle comprises a viral genome comprising SEQ ID NO: 2031 and a capsid comprising SEQ ID NO: 138. In some embodiments, the AAV particle comprises a viral genome comprising SEQ ID NO: 2031 and a capsid encoded by a nucleic acid sequence comprising SEQ ID NO: 137. In some embodiments, the AAV particle comprises a viral genome comprising SEQ ID NO: 2031 and a capsid comprising SEQ ID NO: 5. In some embodiments, the AAV particle comprises a viral genome comprising SEQ ID NO: 2031 and a capsid encoded by a nucleic acid sequence comprising SEQ ID NO: 6. In some embodiments, the AAV particle comprises a viral genome comprising SEQ ID NO: 2031 and a capsid comprising SEQ ID NO: 4. In some embodiments, the AAV particle comprises a viral genome comprising SEQ ID NO: 2031 and a capsid comprising SEQ ID NO: 11. In some embodiments, the AAV particle comprises a viral genome comprising SEQ ID NO: 2031 and a capsid comprising SEQ ID NO: 2679. In some embodiments, the AAV particle comprises a viral genome comprising SEQ ID NO: 2031 and a capsid comprising SEQ ID NO: 2809. In some embodiments, the AAV particle comprises a viral genome comprising SEQ ID NO: 2031 and a capsid comprising SEQ ID NO: 2871.

In some embodiments, the AAV particle comprises a viral genome comprising SEQ ID NO: 2032 and a VOY101 capsid. In some embodiments, the AAV particle comprises a viral genome comprising SEQ ID NO: 2032 and a VOY201 capsid. In some embodiments, the AAV particle comprises a viral genome comprising SEQ ID NO: 2032 and an AAV9 capsid. In some embodiments, the AAV particle comprises a viral genome comprising SEQ ID NO: 2032 and an AAV9K449R capsid. In some embodiments, the AAV particle comprises a viral genome comprising SEQ ID NO: 2032 and an AAVPHP.B capsid. In some embodiments, the AAV particle comprises a viral genome comprising SEQ ID NO: 2032 and an AAVPHP.N capsid. In some embodiments, the AAV particle comprises a viral genome comprising SEQ ID NO: 2032 and an AAV2 variant capsid. In some embodiments, the AAV particle comprises a viral genome comprising SEQ ID NO: 2032 and an AAV2/3 variant capsid. In some embodiments, the AAV particle comprises a viral genome comprising SEQ ID NO: 2032 and a capsid comprising SEQ ID NO: 1. In some embodiments, the AAV particle comprises a viral genome comprising SEQ ID NO: 2032 and a capsid encoded by a nucleic acid sequence comprising SEQ ID NO: 2. In some embodiments, the AAV particle comprises a viral genome comprising SEQ ID NO: 2032 and a capsid comprising SEQ ID NO: 4534. In some embodiments, the AAV particle comprises a viral genome comprising SEQ ID NO: 2032 and a capsid encoded by a nucleic acid sequence comprising SEQ ID NO: 3. In some embodiments, the AAV particle comprises a viral genome comprising SEQ ID NO: 2032 and a capsid comprising SEQ ID NO: 138. In some embodiments, the AAV particle comprises a viral genome comprising SEQ ID NO: 2032 and a capsid encoded by a nucleic acid sequence comprising SEQ ID NO: 137. In some embodiments, the AAV particle comprises a viral genome comprising SEQ ID NO: 2032 and a capsid comprising SEQ ID NO: 5. In some embodiments, the AAV particle comprises a viral genome comprising SEQ ID NO: 2032 and a capsid encoded by a nucleic acid sequence comprising SEQ ID NO: 6. In some embodiments, the AAV particle comprises a viral genome comprising SEQ ID NO: 2032 and a capsid comprising SEQ ID NO: 4. In some embodiments, the AAV particle comprises a viral genome comprising SEQ ID NO: 2032 and a capsid comprising SEQ ID NO: 11. In some embodiments, the AAV particle comprises a viral genome comprising SEQ ID NO: 2032 and a capsid comprising SEQ ID NO: 2679. In some embodiments, the AAV particle comprises a viral genome comprising SEQ ID NO: 2032 and a capsid comprising SEQ ID NO: 2809. In some embodiments, the AAV particle comprises a viral genome comprising SEQ ID NO: 2032 and a capsid comprising SEQ ID NO: 2871.

In some embodiments, the AAV particle comprises a viral genome comprising SEQ ID NO: 2033 and a VOY101 capsid. In some embodiments, the AAV particle comprises a viral genome comprising SEQ ID NO: 2033 and a VOY201 capsid. In some embodiments, the AAV particle comprises a viral genome comprising SEQ ID NO: 2033 and an AAV9 capsid. In some embodiments, the AAV particle comprises a viral genome comprising SEQ ID NO: 2033 and an AAV9K449R capsid. In some embodiments, the AAV particle comprises a viral genome comprising SEQ ID NO: 2033 and an AAVPHP.B capsid. In some embodiments, the AAV particle comprises a viral genome comprising SEQ ID NO: 2033 and an AAVPHP.N capsid. In some embodiments, the AAV particle comprises a viral genome comprising SEQ ID NO: 2033 and an AAV2 variant capsid. In some embodiments, the AAV particle comprises a viral genome comprising SEQ ID NO: 2033 and an AAV2/3 variant capsid. In some embodiments, the AAV particle comprises a viral genome comprising SEQ ID NO: 2033 and a capsid comprising SEQ ID NO: 1. In some embodiments, the AAV particle comprises a viral genome comprising SEQ ID NO: 2033 and a capsid encoded by a nucleic acid sequence comprising SEQ ID NO: 2. In some embodiments, the AAV particle comprises a viral genome comprising SEQ ID NO: 2033 and a capsid comprising SEQ ID NO: 4534. In some embodiments, the AAV particle comprises a viral genome comprising SEQ ID NO: 2033 and a capsid encoded by a nucleic acid sequence comprising SEQ ID NO: 3. In some embodiments, the AAV particle comprises a viral genome comprising SEQ ID NO: 2033 and a capsid comprising SEQ ID NO: 138. In some embodiments, the AAV particle comprises a viral genome comprising SEQ ID NO: 2033 and a capsid encoded by a nucleic acid sequence comprising SEQ ID NO: 137. In some embodiments, the AAV particle comprises a viral genome comprising SEQ ID NO: 2033 and a capsid comprising SEQ ID NO: 5. In some embodiments, the AAV particle comprises a viral genome comprising SEQ ID NO: 2033 and a capsid encoded by a nucleic acid sequence comprising SEQ ID NO: 6. In some embodiments, the AAV particle comprises a viral genome comprising SEQ ID NO: 2033 and a capsid comprising SEQ ID NO: 4. In some embodiments, the AAV particle comprises a viral genome comprising SEQ ID NO: 2033 and a capsid comprising SEQ ID NO: 11. In some embodiments, the AAV particle comprises a viral genome comprising SEQ ID NO: 2033 and a capsid comprising SEQ ID NO: 2679. In some embodiments, the AAV particle comprises a viral genome comprising SEQ ID NO: 2033 and a capsid comprising SEQ ID NO: 2809. In some embodiments, the AAV particle comprises a viral genome comprising SEQ ID NO: 2033 and a capsid comprising SEQ ID NO: 2871.

In some embodiments, the AAV particle comprises a viral genome comprising SEQ ID NO: 2034 and a VOY101 capsid. In some embodiments, the AAV particle comprises a viral genome comprising SEQ ID NO: 2034 and a VOY201 capsid. In some embodiments, the AAV particle comprises a viral genome comprising SEQ ID NO: 2034 and an AAV9 capsid. In some embodiments, the AAV particle comprises a viral genome comprising SEQ ID NO: 2034 and an AAV9K449R capsid. In some embodiments, the AAV particle comprises a viral genome comprising SEQ ID NO: 2034 and an AAVPHP.B capsid. In some embodiments, the AAV particle comprises a viral genome comprising SEQ ID NO: 2034 and an AAVPHP.N capsid. In some embodiments, the AAV particle comprises a viral genome comprising SEQ ID NO: 2034 and an AAV2 variant capsid. In some embodiments, the AAV particle comprises a viral genome comprising SEQ ID NO: 2034 and an AAV2/3 variant capsid. In some embodiments, the AAV particle comprises a viral genome comprising SEQ ID NO: 2034 and a capsid comprising SEQ ID NO: 1. In some embodiments, the AAV particle comprises a viral genome comprising SEQ ID NO: 2034 and a capsid encoded by a nucleic acid sequence comprising SEQ ID NO: 2. In some embodiments, the AAV particle comprises a viral genome comprising SEQ ID NO: 2034 and a capsid comprising SEQ ID NO: 4534. In some embodiments, the AAV particle comprises a viral genome comprising SEQ ID NO: 2034 and a capsid encoded by a nucleic acid sequence comprising SEQ ID NO: 3. In some embodiments, the AAV particle comprises a viral genome comprising SEQ ID NO: 2034 and a capsid comprising SEQ ID NO: 138. In some embodiments, the AAV particle comprises a viral genome comprising SEQ ID NO: 2034 and a capsid encoded by a nucleic acid sequence comprising SEQ ID NO: 137. In some embodiments, the AAV particle comprises a viral genome comprising SEQ ID NO: 2034 and a capsid comprising SEQ ID NO: 5. In some embodiments, the AAV particle comprises a viral genome comprising SEQ ID NO: 2034 and a capsid encoded by a nucleic acid sequence comprising SEQ ID NO: 6. In some embodiments, the AAV particle comprises a viral genome comprising SEQ ID NO: 2034 and a capsid comprising SEQ ID NO: 4. In some embodiments, the AAV particle comprises a viral genome comprising SEQ ID NO: 2034 and a capsid comprising SEQ ID NO: 11. In some embodiments, the AAV particle comprises a viral genome comprising SEQ ID NO: 2034 and a capsid comprising SEQ ID NO: 2679. In some embodiments, the AAV particle comprises a viral genome comprising SEQ ID NO: 2034 and a capsid comprising SEQ ID NO: 2809. In some embodiments, the AAV particle comprises a viral genome comprising SEQ ID NO: 2034 and a capsid comprising SEQ ID NO: 2871.

In some embodiments, the AAV particle comprises a viral genome comprising SEQ ID NO: 2035 and a VOY101 capsid. In some embodiments, the AAV particle comprises a viral genome comprising SEQ ID NO: 2035 and a VOY201 capsid. In some embodiments, the AAV particle comprises a viral genome comprising SEQ ID NO: 2035 and an AAV9 capsid. In some embodiments, the AAV particle comprises a viral genome comprising SEQ ID NO: 2035 and an AAV9K449R capsid. In some embodiments, the AAV particle comprises a viral genome comprising SEQ ID NO: 2035 and an AAVPHP.B capsid. In some embodiments, the AAV particle comprises a viral genome comprising SEQ ID NO: 2035 and an AAVPHP.N capsid. In some embodiments, the AAV particle comprises a viral genome comprising SEQ ID NO: 2035 and an AAV2 variant capsid. In some embodiments, the AAV particle comprises a viral genome comprising SEQ ID NO: 2035 and an AAV2/3 variant capsid. In some embodiments, the AAV particle comprises a viral genome comprising SEQ ID NO: 2035 and a capsid comprising SEQ ID NO: 1. In some embodiments, the AAV particle comprises a viral genome comprising SEQ ID NO: 2035 and a capsid encoded by a nucleic acid sequence comprising SEQ ID NO: 2. In some embodiments, the AAV particle comprises a viral genome comprising SEQ ID NO: 2035 and a capsid comprising SEQ ID NO: 4534. In some embodiments, the AAV particle comprises a viral genome comprising SEQ ID NO: 2035 and a capsid encoded by a nucleic acid sequence comprising SEQ ID NO: 3. In some embodiments, the AAV particle comprises a viral genome comprising SEQ ID NO: 2035 and a capsid comprising SEQ ID NO: 138. In some embodiments, the AAV particle comprises a viral genome comprising SEQ ID NO: 2035 and a capsid encoded by a nucleic acid sequence comprising SEQ ID NO: 137. In some embodiments, the AAV particle comprises a viral genome comprising SEQ ID NO: 2035 and a capsid comprising SEQ ID NO: 5. In some embodiments, the AAV particle comprises a viral genome comprising SEQ ID NO: 2035 and a capsid encoded by a nucleic acid sequence comprising SEQ ID NO: 6. In some embodiments, the AAV particle comprises a viral genome comprising SEQ ID NO: 2035 and a capsid comprising SEQ ID NO: 4. In some embodiments, the AAV particle comprises a viral genome comprising SEQ ID NO: 2035 and a capsid comprising SEQ ID NO: 11. In some embodiments, the AAV particle comprises a viral genome comprising SEQ ID NO: 2035 and a capsid comprising SEQ ID NO: 2679. In some embodiments, the AAV particle comprises a viral genome comprising SEQ ID NO: 2035 and a capsid comprising SEQ ID NO: 2809. In some embodiments, the AAV particle comprises a viral genome comprising SEQ ID NO: 2035 and a capsid comprising SEQ ID NO: 2871.

In some embodiments, the AAV particle comprises a viral genome comprising SEQ ID NO: 2036 and a VOY101 capsid. In some embodiments, the AAV particle comprises a viral genome comprising SEQ ID NO: 2036 and a VOY201 capsid. In some embodiments, the AAV particle comprises a viral genome comprising SEQ ID NO: 2036 and an AAV9 capsid. In some embodiments, the AAV particle comprises a viral genome comprising SEQ ID NO: 2036 and an AAV9K449R capsid. In some embodiments, the AAV particle comprises a viral genome comprising SEQ ID NO: 2036 and an AAVPHP.B capsid. In some embodiments, the AAV particle comprises a viral genome comprising SEQ ID NO: 2036 and an AAVPHP.N capsid. In some embodiments, the AAV particle comprises a viral genome comprising SEQ ID NO: 2036 and an AAV2 variant capsid. In some embodiments, the AAV particle comprises a viral genome comprising SEQ ID NO: 2036 and an AAV2/3 variant capsid. In some embodiments, the AAV particle comprises a viral genome comprising SEQ ID NO: 2036 and a capsid comprising SEQ ID NO: 1. In some embodiments, the AAV particle comprises a viral genome comprising SEQ ID NO: 2036 and a capsid encoded by a nucleic acid sequence comprising SEQ ID NO: 2. In some embodiments, the AAV particle comprises a viral genome comprising SEQ ID NO: 2036 and a capsid comprising SEQ ID NO: 4534. In some embodiments, the AAV particle comprises a viral genome comprising SEQ ID NO: 2036 and a capsid encoded by a nucleic acid sequence comprising SEQ ID NO: 3. In some embodiments, the AAV particle comprises a viral genome comprising SEQ ID NO: 2036 and a capsid comprising SEQ ID NO: 138. In some embodiments, the AAV particle comprises a viral genome comprising SEQ ID NO: 2036 and a capsid encoded by a nucleic acid sequence comprising SEQ ID NO: 137. In some embodiments, the AAV particle comprises a viral genome comprising SEQ ID NO: 2036 and a capsid comprising SEQ ID NO: 5. In some embodiments, the AAV particle comprises a viral genome comprising SEQ ID NO: 2036 and a capsid encoded by a nucleic acid sequence comprising SEQ ID NO: 6. In some embodiments, the AAV particle comprises a viral genome comprising SEQ ID NO: 2036 and a capsid comprising SEQ ID NO: 4. In some embodiments, the AAV particle comprises a viral genome comprising SEQ ID NO: 2036 and a capsid comprising SEQ ID NO: 11. In some embodiments, the AAV particle comprises a viral genome comprising SEQ ID NO: 2036 and a capsid comprising SEQ ID NO: 2679. In some embodiments, the AAV particle comprises a viral genome comprising SEQ ID NO: 2036 and a capsid comprising SEQ ID NO: 2809. In some embodiments, the AAV particle comprises a viral genome comprising SEQ ID NO: 2036 and a capsid comprising SEQ ID NO: 2871.

In some embodiments, the AAV particle comprises a viral genome comprising SEQ ID NO: 2037 and a VOY101 capsid. In some embodiments, the AAV particle comprises a viral genome comprising SEQ ID NO: 2037 and a VOY201 capsid. In some embodiments, the AAV particle comprises a viral genome comprising SEQ ID NO: 2037 and an AAV9 capsid. In some embodiments, the AAV particle comprises a viral genome comprising SEQ ID NO: 2037 and an AAV9K449R capsid. In some embodiments, the AAV particle comprises a viral genome comprising SEQ ID NO: 2037 and an AAVPHP.B capsid. In some embodiments, the AAV particle comprises a viral genome comprising SEQ ID NO: 2037 and an AAVPHP.N capsid. In some embodiments, the AAV particle comprises a viral genome comprising SEQ ID NO: 2037 and an AAV2 variant capsid. In some embodiments, the AAV particle comprises a viral genome comprising SEQ ID NO: 2037 and an AAV2/3 variant capsid. In some embodiments, the AAV particle comprises a viral genome comprising SEQ ID NO: 2037 and a capsid comprising SEQ ID NO: 1. In some embodiments, the AAV particle comprises a viral genome comprising SEQ ID NO: 2037 and a capsid encoded by a nucleic acid sequence comprising SEQ ID NO: 2. In some embodiments, the AAV particle comprises a viral genome comprising SEQ ID NO: 2037 and a capsid comprising SEQ ID NO: 4534. In some embodiments, the AAV particle comprises a viral genome comprising SEQ ID NO: 2037 and a capsid encoded by a nucleic acid sequence comprising SEQ ID NO: 3. In some embodiments, the AAV particle comprises a viral genome comprising SEQ ID NO: 2037 and a capsid comprising SEQ ID NO: 138. In some embodiments, the AAV particle comprises a viral genome comprising SEQ ID NO: 2037 and a capsid encoded by a nucleic acid sequence comprising SEQ ID NO: 137. In some embodiments, the AAV particle comprises a viral genome comprising SEQ ID NO: 2037 and a capsid comprising SEQ ID NO: 5. In some embodiments, the AAV particle comprises a viral genome comprising SEQ ID NO: 2037 and a capsid encoded by a nucleic acid sequence comprising SEQ ID NO: 6. In some embodiments, the AAV particle comprises a viral genome comprising SEQ ID NO: 2037 and a capsid comprising SEQ ID NO: 4. In some embodiments, the AAV particle comprises a viral genome comprising SEQ ID NO: 2037 and a capsid comprising SEQ ID NO: 11. In some embodiments, the AAV particle comprises a viral genome comprising SEQ ID NO: 2037 and a capsid comprising SEQ ID NO: 2679. In some embodiments, the AAV particle comprises a viral genome comprising SEQ ID NO: 2037 and a capsid comprising SEQ ID NO: 2809. In some embodiments, the AAV particle comprises a viral genome comprising SEQ ID NO: 2037 and a capsid comprising SEQ ID NO: 2871.

In some embodiments, the AAV particle comprises a viral genome comprising SEQ ID NO: 2038 and a VOY101 capsid. In some embodiments, the AAV particle comprises a viral genome comprising SEQ ID NO: 2038 and a VOY201 capsid. In some embodiments, the AAV particle comprises a viral genome comprising SEQ ID NO: 2038 and an AAV9 capsid. In some embodiments, the AAV particle comprises a viral genome comprising SEQ ID NO: 2038 and an AAV9K449R capsid. In some embodiments, the AAV particle comprises a viral genome comprising SEQ ID NO: 2038 and an AAVPHP.B capsid. In some embodiments, the AAV particle comprises a viral genome comprising SEQ ID NO: 2038 and an AAVPHP.N capsid. In some embodiments, the AAV particle comprises a viral genome comprising SEQ ID NO: 2038 and an AAV2 variant capsid. In some embodiments, the AAV particle comprises a viral genome comprising SEQ ID NO: 2038 and an AAV2/3 variant capsid. In some embodiments, the AAV particle comprises a viral genome comprising SEQ ID NO: 2038 and a capsid comprising SEQ ID NO: 1. In some embodiments, the AAV particle comprises a viral genome comprising SEQ ID NO: 2038 and a capsid encoded by a nucleic acid sequence comprising SEQ ID NO: 2. In some embodiments, the AAV particle comprises a viral genome comprising SEQ ID NO: 2038 and a capsid comprising SEQ ID NO: 4534. In some embodiments, the AAV particle comprises a viral genome comprising SEQ ID NO: 2038 and a capsid encoded by a nucleic acid sequence comprising SEQ ID NO: 3. In some embodiments, the AAV particle comprises a viral genome comprising SEQ ID NO: 2038 and a capsid comprising SEQ ID NO: 138. In some embodiments, the AAV particle comprises a viral genome comprising SEQ ID NO: 2038 and a capsid encoded by a nucleic acid sequence comprising SEQ ID NO: 137. In some embodiments, the AAV particle comprises a viral genome comprising SEQ ID NO: 2038 and a capsid comprising SEQ ID NO: 5. In some embodiments, the AAV particle comprises a viral genome comprising SEQ ID NO: 2038 and a capsid encoded by a nucleic acid sequence comprising SEQ ID NO: 6. In some embodiments, the AAV particle comprises a viral genome comprising SEQ ID NO: 2038 and a capsid comprising SEQ ID NO: 4. In some embodiments, the AAV particle comprises a viral genome comprising SEQ ID NO: 2038 and a capsid comprising SEQ ID NO: 11. In some embodiments, the AAV particle comprises a viral genome comprising SEQ ID NO: 2038 and a capsid comprising SEQ ID NO: 2679. In some embodiments, the AAV particle comprises a viral genome comprising SEQ ID NO: 2038 and a capsid comprising SEQ ID NO: 2809. In some embodiments, the AAV particle comprises a viral genome comprising SEQ ID NO: 2038 and a capsid comprising SEQ ID NO: 2871.

In some embodiments, the AAV particle comprises a viral genome comprising SEQ ID NO: 2039 and a VOY101 capsid. In some embodiments, the AAV particle comprises a viral genome comprising SEQ ID NO: 2039 and a VOY201 capsid. In some embodiments, the AAV particle comprises a viral genome comprising SEQ ID NO: 2039 and an AAV9 capsid. In some embodiments, the AAV particle comprises a viral genome comprising SEQ ID NO: 2039 and an AAV9K449R capsid. In some embodiments, the AAV particle comprises a viral genome comprising SEQ ID NO: 2039 and an AAVPHP.B capsid. In some embodiments, the AAV particle comprises a viral genome comprising SEQ ID NO: 2039 and an AAVPHP.N capsid. In some embodiments, the AAV particle comprises a viral genome comprising SEQ ID NO: 2039 and an AAV2 variant capsid. In some embodiments, the AAV particle comprises a viral genome comprising SEQ ID NO: 2039 and an AAV2/3 variant capsid. In some embodiments, the AAV particle comprises a viral genome comprising SEQ ID NO: 2039 and a capsid comprising SEQ ID NO: 1. In some embodiments, the AAV particle comprises a viral genome comprising SEQ ID NO: 2039 and a capsid encoded by a nucleic acid sequence comprising SEQ ID NO: 2. In some embodiments, the AAV particle comprises a viral genome comprising SEQ ID NO: 2039 and a capsid comprising SEQ ID NO: 4534. In some embodiments, the AAV particle comprises a viral genome comprising SEQ ID NO: 2039 and a capsid encoded by a nucleic acid sequence comprising SEQ ID NO: 3. In some embodiments, the AAV particle comprises a viral genome comprising SEQ ID NO: 2039 and a capsid comprising SEQ ID NO: 138. In some embodiments, the AAV particle comprises a viral genome comprising SEQ ID NO: 2039 and a capsid encoded by a nucleic acid sequence comprising SEQ ID NO: 137. In some embodiments, the AAV particle comprises a viral genome comprising SEQ ID NO: 2039 and a capsid comprising SEQ ID NO: 5. In some embodiments, the AAV particle comprises a viral genome comprising SEQ ID NO: 2039 and a capsid encoded by a nucleic acid sequence comprising SEQ ID NO: 6. In some embodiments, the AAV particle comprises a viral genome comprising SEQ ID NO: 2039 and a capsid comprising SEQ ID NO: 4. In some embodiments, the AAV particle comprises a viral genome comprising SEQ ID NO: 2039 and a capsid comprising SEQ ID NO: 11. In some embodiments, the AAV particle comprises a viral genome comprising SEQ ID NO: 2039 and a capsid comprising SEQ ID NO: 2679. In some embodiments, the AAV particle comprises a viral genome comprising SEQ ID NO: 2039 and a capsid comprising SEQ ID NO: 2809. In some embodiments, the AAV particle comprises a viral genome comprising SEQ ID NO: 2039 and a capsid comprising SEQ ID NO: 2871.

In some embodiments, the AAV particle comprises a viral genome comprising SEQ ID NO: 2040 and a VOY101 capsid. In some embodiments, the AAV particle comprises a viral genome comprising SEQ ID NO: 2040 and a VOY201 capsid. In some embodiments, the AAV particle comprises a viral genome comprising SEQ ID NO: 2040 and an AAV9 capsid. In some embodiments, the AAV particle comprises a viral genome comprising SEQ ID NO: 2040 and an AAV9K449R capsid. In some embodiments, the AAV particle comprises a viral genome comprising SEQ ID NO: 2040 and an AAVPHP.B capsid. In some embodiments, the AAV particle comprises a viral genome comprising SEQ ID NO: 2040 and an AAVPHP.N capsid. In some embodiments, the AAV particle comprises a viral genome comprising SEQ ID NO: 2040 and an AAV2 variant capsid. In some embodiments, the AAV particle comprises a viral genome comprising SEQ ID NO: 2040 and an AAV2/3 variant capsid. In some embodiments, the AAV particle comprises a viral genome comprising SEQ ID NO: 2040 and a capsid comprising SEQ ID NO: 1. In some embodiments, the AAV particle comprises a viral genome comprising SEQ ID NO: 2040 and a capsid encoded by a nucleic acid sequence comprising SEQ ID NO: 2. In some embodiments, the AAV particle comprises a viral genome comprising SEQ ID NO: 2040 and a capsid comprising SEQ ID NO: 4534. In some embodiments, the AAV particle comprises a viral genome comprising SEQ ID NO: 2040 and a capsid encoded by a nucleic acid sequence comprising SEQ ID NO: 3. In some embodiments, the AAV particle comprises a viral genome comprising SEQ ID NO: 2040 and a capsid comprising SEQ ID NO: 138. In some embodiments, the AAV particle comprises a viral genome comprising SEQ ID NO: 2040 and a capsid encoded by a nucleic acid sequence comprising SEQ ID NO: 137. In some embodiments, the AAV particle comprises a viral genome comprising SEQ ID NO: 2040 and a capsid comprising SEQ ID NO: 5. In some embodiments, the AAV particle comprises a viral genome comprising SEQ ID NO: 2040 and a capsid encoded by a nucleic acid sequence comprising SEQ ID NO: 6. In some embodiments, the AAV particle comprises a viral genome comprising SEQ ID NO: 2040 and a capsid comprising SEQ ID NO: 4. In some embodiments, the AAV particle comprises a viral genome comprising SEQ ID NO: 2040 and a capsid comprising SEQ ID NO: 11. In some embodiments, the AAV particle comprises a viral genome comprising SEQ ID NO: 2040 and a capsid comprising SEQ ID NO: 2679. In some embodiments, the AAV particle comprises a viral genome comprising SEQ ID NO: 2040 and a capsid comprising SEQ ID NO: 2809. In some embodiments, the AAV particle comprises a viral genome comprising SEQ ID NO: 2040 and a capsid comprising SEQ ID NO: 2871.

In some embodiments, the AAV particle comprises a viral genome comprising SEQ ID NO: 2041 and a VOY101 capsid. In some embodiments, the AAV particle comprises a viral genome comprising SEQ ID NO: 2041 and a VOY201 capsid. In some embodiments, the AAV particle comprises a viral genome comprising SEQ ID NO: 2041 and an AAV9 capsid. In some embodiments, the AAV particle comprises a viral genome comprising SEQ ID NO: 2041 and an AAV9K449R capsid. In some embodiments, the AAV particle comprises a viral genome comprising SEQ ID NO: 2041 and an AAVPHP.B capsid. In some embodiments, the AAV particle comprises a viral genome comprising SEQ ID NO: 2041 and an AAVPHP.N capsid. In some embodiments, the AAV particle comprises a viral genome comprising SEQ ID NO: 2041 and an AAV2 variant capsid. In some embodiments, the AAV particle comprises a viral genome comprising SEQ ID NO: 2041 and an AAV2/3 variant capsid. In some embodiments, the AAV particle comprises a viral genome comprising SEQ ID NO: 2041 and a capsid comprising SEQ ID NO: 1. In some embodiments, the AAV particle comprises a viral genome comprising SEQ ID NO: 2041 and a capsid encoded by a nucleic acid sequence comprising SEQ ID NO: 2. In some embodiments, the AAV particle comprises a viral genome comprising SEQ ID NO: 2041 and a capsid comprising SEQ ID NO: 4534. In some embodiments, the AAV particle comprises a viral genome comprising SEQ ID NO: 2041 and a capsid encoded by a nucleic acid sequence comprising SEQ ID NO: 3. In some embodiments, the AAV particle comprises a viral genome comprising SEQ ID NO: 2041 and a capsid comprising SEQ ID NO: 138. In some embodiments, the AAV particle comprises a viral genome comprising SEQ ID NO: 2041 and a capsid encoded by a nucleic acid sequence comprising SEQ ID NO: 137. In some embodiments, the AAV particle comprises a viral genome comprising SEQ ID NO: 2041 and a capsid comprising SEQ ID NO: 5. In some embodiments, the AAV particle comprises a viral genome comprising SEQ ID NO: 2041 and a capsid encoded by a nucleic acid sequence comprising SEQ ID NO: 6. In some embodiments, the AAV particle comprises a viral genome comprising SEQ ID NO: 2041 and a capsid comprising SEQ ID NO: 4. In some embodiments, the AAV particle comprises a viral genome comprising SEQ ID NO: 2041 and a capsid comprising SEQ ID NO: 11. In some embodiments, the AAV particle comprises a viral genome comprising SEQ ID NO: 2041 and a capsid comprising SEQ ID NO: 2679. In some embodiments, the AAV particle comprises a viral genome comprising SEQ ID NO: 2041 and a capsid comprising SEQ ID NO: 2809. In some embodiments, the AAV particle comprises a viral genome comprising SEQ ID NO: 2041 and a capsid comprising SEQ ID NO: 2871.

In some embodiments, the AAV particle comprises a viral genome comprising SEQ ID NO: 2042 and a VOY101 capsid. In some embodiments, the AAV particle comprises a viral genome comprising SEQ ID NO: 2042 and a VOY201 capsid. In some embodiments, the AAV particle comprises a viral genome comprising SEQ ID NO: 2042 and an AAV9 capsid. In some embodiments, the AAV particle comprises a viral genome comprising SEQ ID NO: 2042 and an AAV9K449R capsid. In some embodiments, the AAV particle comprises a viral genome comprising SEQ ID NO: 2042 and an AAVPHP.B capsid. In some embodiments, the AAV particle comprises a viral genome comprising SEQ ID NO: 2042 and an AAVPHP.N capsid. In some embodiments, the AAV particle comprises a viral genome comprising SEQ ID NO: 2042 and an AAV2 variant capsid. In some embodiments, the AAV particle comprises a viral genome comprising SEQ ID NO: 2042 and an AAV2/3 variant capsid. In some embodiments, the AAV particle comprises a viral genome comprising SEQ ID NO: 2042 and a capsid comprising SEQ ID NO: 1. In some embodiments, the AAV particle comprises a viral genome comprising SEQ ID NO: 2042 and a capsid encoded by a nucleic acid sequence comprising SEQ ID NO: 2. In some embodiments, the AAV particle comprises a viral genome comprising SEQ ID NO: 2042 and a capsid comprising SEQ ID NO: 4534. In some embodiments, the AAV particle comprises a viral genome comprising SEQ ID NO: 2042 and a capsid encoded by a nucleic acid sequence comprising SEQ ID NO: 3. In some embodiments, the AAV particle comprises a viral genome comprising SEQ ID NO: 2042 and a capsid comprising SEQ ID NO: 138. In some embodiments, the AAV particle comprises a viral genome comprising SEQ ID NO: 2042 and a capsid encoded by a nucleic acid sequence comprising SEQ ID NO: 137. In some embodiments, the AAV particle comprises a viral genome comprising SEQ ID NO: 2042 and a capsid comprising SEQ ID NO: 5. In some embodiments, the AAV particle comprises a viral genome comprising SEQ ID NO: 2042 and a capsid encoded by a nucleic acid sequence comprising SEQ ID NO: 6. In some embodiments, the AAV particle comprises a viral genome comprising SEQ ID NO: 2042 and a capsid comprising SEQ ID NO: 4. In some embodiments, the AAV particle comprises a viral genome comprising SEQ ID NO: 2042 and a capsid comprising SEQ ID NO: 11. In some embodiments, the AAV particle comprises a viral genome comprising SEQ ID NO: 2042 and a capsid comprising SEQ ID NO: 2679. In some embodiments, the AAV particle comprises a viral genome comprising SEQ ID NO: 2042 and a capsid comprising SEQ ID NO: 2809. In some embodiments, the AAV particle comprises a viral genome comprising SEQ ID NO: 2042 and a capsid comprising SEQ ID NO: 2871.

In some embodiments, the AAV particle comprises a viral genome comprising SEQ ID NO: 2043 and a VOY101 capsid. In some embodiments, the AAV particle comprises a viral genome comprising SEQ ID NO: 2043 and a VOY201 capsid. In some embodiments, the AAV particle comprises a viral genome comprising SEQ ID NO: 2043 and an AAV9 capsid. In some embodiments, the AAV particle comprises a viral genome comprising SEQ ID NO: 2043 and an AAV9K449R capsid. In some embodiments, the AAV particle comprises a viral genome comprising SEQ ID NO: 2043 and an AAVPHP.B capsid. In some embodiments, the AAV particle comprises a viral genome comprising SEQ ID NO: 2043 and an AAVPHP.N capsid. In some embodiments, the AAV particle comprises a viral genome comprising SEQ ID NO: 2043 and an AAV2 variant capsid. In some embodiments, the AAV particle comprises a viral genome comprising SEQ ID NO: 2043 and an AAV2/3 variant capsid. In some embodiments, the AAV particle comprises a viral genome comprising SEQ ID NO: 2043 and a capsid comprising SEQ ID NO: 1. In some embodiments, the AAV particle comprises a viral genome comprising SEQ ID NO: 2043 and a capsid encoded by a nucleic acid sequence comprising SEQ ID NO: 2. In some embodiments, the AAV particle comprises a viral genome comprising SEQ ID NO: 2043 and a capsid comprising SEQ ID NO: 4534. In some embodiments, the AAV particle comprises a viral genome comprising SEQ ID NO: 2043 and a capsid encoded by a nucleic acid sequence comprising SEQ ID NO: 3. In some embodiments, the AAV particle comprises a viral genome comprising SEQ ID NO: 2043 and a capsid comprising SEQ ID NO: 138. In some embodiments, the AAV particle comprises a viral genome comprising SEQ ID NO: 2043 and a capsid encoded by a nucleic acid sequence comprising SEQ ID NO: 137. In some embodiments, the AAV particle comprises a viral genome comprising SEQ ID NO: 2043 and a capsid comprising SEQ ID NO: 5. In some embodiments, the AAV particle comprises a viral genome comprising SEQ ID NO: 2043 and a capsid encoded by a nucleic acid sequence comprising SEQ ID NO: 6. In some embodiments, the AAV particle comprises a viral genome comprising SEQ ID NO: 2043 and a capsid comprising SEQ ID NO: 4. In some embodiments, the AAV particle comprises a viral genome comprising SEQ ID NO: 2043 and a capsid comprising SEQ ID NO: 11. In some embodiments, the AAV particle comprises a viral genome comprising SEQ ID NO: 2043 and a capsid comprising SEQ ID NO: 2679. In some embodiments, the AAV particle comprises a viral genome comprising SEQ ID NO: 2043 and a capsid comprising SEQ ID NO: 2809. In some embodiments, the AAV particle comprises a viral genome comprising SEQ ID NO: 2043 and a capsid comprising SEQ ID NO: 2871.

In some embodiments, the AAV particle comprises a viral genome comprising SEQ ID NO: 2044 and a VOY101 capsid. In some embodiments, the AAV particle comprises a viral genome comprising SEQ ID NO: 2044 and a VOY201 capsid. In some embodiments, the AAV particle comprises a viral genome comprising SEQ ID NO: 2044 and an AAV9 capsid. In some embodiments, the AAV particle comprises a viral genome comprising SEQ ID NO: 2044 and an AAV9K449R capsid. In some embodiments, the AAV particle comprises a viral genome comprising SEQ ID NO: 2044 and an AAVPHP.B capsid. In some embodiments, the AAV particle comprises a viral genome comprising SEQ ID NO: 2044 and an AAVPHP.N capsid. In some embodiments, the AAV particle comprises a viral genome comprising SEQ ID NO: 2044 and an AAV2 variant capsid. In some embodiments, the AAV particle comprises a viral genome comprising SEQ ID NO: 2044 and an AAV2/3 variant capsid. In some embodiments, the AAV particle comprises a viral genome comprising SEQ ID NO: 2044 and a capsid comprising SEQ ID NO: 1. In some embodiments, the AAV particle comprises a viral genome comprising SEQ ID NO: 2044 and a capsid encoded by a nucleic acid sequence comprising SEQ ID NO: 2. In some embodiments, the AAV particle comprises a viral genome comprising SEQ ID NO: 2044 and a capsid comprising SEQ ID NO: 4534. In some embodiments, the AAV particle comprises a viral genome comprising SEQ ID NO: 2044 and a capsid encoded by a nucleic acid sequence comprising SEQ ID NO: 3. In some embodiments, the AAV particle comprises a viral genome comprising SEQ ID NO: 2044 and a capsid comprising SEQ ID NO: 138. In some embodiments, the AAV particle comprises a viral genome comprising SEQ ID NO: 2044 and a capsid encoded by a nucleic acid sequence comprising SEQ ID NO: 137. In some embodiments, the AAV particle comprises a viral genome comprising SEQ ID NO: 2044 and a capsid comprising SEQ ID NO: 5. In some embodiments, the AAV particle comprises a viral genome comprising SEQ ID NO: 2044 and a capsid encoded by a nucleic acid sequence comprising SEQ ID NO: 6. In some embodiments, the AAV particle comprises a viral genome comprising SEQ ID NO: 2044 and a capsid comprising SEQ ID NO: 4. In some embodiments, the AAV particle comprises a viral genome comprising SEQ ID NO: 2044 and a capsid comprising SEQ ID NO: 11. In some embodiments, the AAV particle comprises a viral genome comprising SEQ ID NO: 2044 and a capsid comprising SEQ ID NO: 2679. In some embodiments, the AAV particle comprises a viral genome comprising SEQ ID NO: 2044 and a capsid comprising SEQ ID NO: 2809. In some embodiments, the AAV particle comprises a viral genome comprising SEQ ID NO: 2044 and a capsid comprising SEQ ID NO: 2871.

In some embodiments, the AAV particle comprises a viral genome comprising SEQ ID NO: 2045 and a VOY101 capsid. In some embodiments, the AAV particle comprises a viral genome comprising SEQ ID NO: 2045 and a VOY201 capsid. In some embodiments, the AAV particle comprises a viral genome comprising SEQ ID NO: 2045 and an AAV9 capsid. In some embodiments, the AAV particle comprises a viral genome comprising SEQ ID NO: 2045 and an AAV9K449R capsid. In some embodiments, the AAV particle comprises a viral genome comprising SEQ ID NO: 2045 and an AAVPHP.B capsid. In some embodiments, the AAV particle comprises a viral genome comprising SEQ ID NO: 2045 and an AAVPHP.N capsid. In some embodiments, the AAV particle comprises a viral genome comprising SEQ ID NO: 2045 and an AAV2 variant capsid. In some embodiments, the AAV particle comprises a viral genome comprising SEQ ID NO: 2045 and an AAV2/3 variant capsid. In some embodiments, the AAV particle comprises a viral genome comprising SEQ ID NO: 2045 and a capsid comprising SEQ ID NO: 1. In some embodiments, the AAV particle comprises a viral genome comprising SEQ ID NO: 2045 and a capsid encoded by a nucleic acid sequence comprising SEQ ID NO: 2. In some embodiments, the AAV particle comprises a viral genome comprising SEQ ID NO: 2045 and a capsid comprising SEQ ID NO: 4534. In some embodiments, the AAV particle comprises a viral genome comprising SEQ ID NO: 2045 and a capsid encoded by a nucleic acid sequence comprising SEQ ID NO: 3. In some embodiments, the AAV particle comprises a viral genome comprising SEQ ID NO: 2045 and a capsid comprising SEQ ID NO: 138. In some embodiments, the AAV particle comprises a viral genome comprising SEQ ID NO: 2045 and a capsid encoded by a nucleic acid sequence comprising SEQ ID NO: 137. In some embodiments, the AAV particle comprises a viral genome comprising SEQ ID NO: 2045 and a capsid comprising SEQ ID NO: 5. In some embodiments, the AAV particle comprises a viral genome comprising SEQ ID NO: 2045 and a capsid encoded by a nucleic acid sequence comprising SEQ ID NO: 6. In some embodiments, the AAV particle comprises a viral genome comprising SEQ ID NO: 2045 and a capsid comprising SEQ ID NO: 4. In some embodiments, the AAV particle comprises a viral genome comprising SEQ ID NO: 2045 and a capsid comprising SEQ ID NO: 11. In some embodiments, the AAV particle comprises a viral genome comprising SEQ ID NO: 2045 and a capsid comprising SEQ ID NO: 2679. In some embodiments, the AAV particle comprises a viral genome comprising SEQ ID NO: 2045 and a capsid comprising SEQ ID NO: 2809. In some embodiments, the AAV particle comprises a viral genome comprising SEQ ID NO: 2045 and a capsid comprising SEQ ID NO: 2871.

In some embodiments, the AAV particle comprises a viral genome comprising SEQ ID NO: 2046 and a VOY101 capsid. In some embodiments, the AAV particle comprises a viral genome comprising SEQ ID NO: 2046 and a VOY201 capsid. In some embodiments, the AAV particle comprises a viral genome comprising SEQ ID NO: 2046 and an AAV9 capsid. In some embodiments, the AAV particle comprises a viral genome comprising SEQ ID NO: 2046 and an AAV9K449R capsid. In some embodiments, the AAV particle comprises a viral genome comprising SEQ ID NO: 2046 and an AAVPHP.B capsid. In some embodiments, the AAV particle comprises a viral genome comprising SEQ ID NO: 2046 and an AAVPHP.N capsid. In some embodiments, the AAV particle comprises a viral genome comprising SEQ ID NO: 2046 and an AAV2 variant capsid. In some embodiments, the AAV particle comprises a viral genome comprising SEQ ID NO: 2046 and an AAV2/3 variant capsid. In some embodiments, the AAV particle comprises a viral genome comprising SEQ ID NO: 2046 and a capsid comprising SEQ ID NO: 1. In some embodiments, the AAV particle comprises a viral genome comprising SEQ ID NO: 2046 and a capsid encoded by a nucleic acid sequence comprising SEQ ID NO: 2. In some embodiments, the AAV particle comprises a viral genome comprising SEQ ID NO: 2046 and a capsid comprising SEQ ID NO: 4534. In some embodiments, the AAV particle comprises a viral genome comprising SEQ ID NO: 2046 and a capsid encoded by a nucleic acid sequence comprising SEQ ID NO: 3. In some embodiments, the AAV particle comprises a viral genome comprising SEQ ID NO: 2046 and a capsid comprising SEQ ID NO: 138. In some embodiments, the AAV particle comprises a viral genome comprising SEQ ID NO: 2046 and a capsid encoded by a nucleic acid sequence comprising SEQ ID NO: 137. In some embodiments, the AAV particle comprises a viral genome comprising SEQ ID NO: 2046 and a capsid comprising SEQ ID NO: 5. In some embodiments, the AAV particle comprises a viral genome comprising SEQ ID NO: 2046 and a capsid encoded by a nucleic acid sequence comprising SEQ ID NO: 6. In some embodiments, the AAV particle comprises a viral genome comprising SEQ ID NO: 2046 and a capsid comprising SEQ ID NO: 4. In some embodiments, the AAV particle comprises a viral genome comprising SEQ ID NO: 2046 and a capsid comprising SEQ ID NO: 11. In some embodiments, the AAV particle comprises a viral genome comprising SEQ ID NO: 2046 and a capsid comprising SEQ ID NO: 2679. In some embodiments, the AAV particle comprises a viral genome comprising SEQ ID NO: 2046 and a capsid comprising SEQ ID NO: 2809. In some embodiments, the AAV particle comprises a viral genome comprising SEQ ID NO: 2046 and a capsid comprising SEQ ID NO: 2871.

In some embodiments, the AAV particle comprises a viral genome comprising SEQ ID NO: 2047 and a VOY101 capsid. In some embodiments, the AAV particle comprises a viral genome comprising SEQ ID NO: 2047 and a VOY201 capsid. In some embodiments, the AAV particle comprises a viral genome comprising SEQ ID NO: 2047 and an AAV9 capsid. In some embodiments, the AAV particle comprises a viral genome comprising SEQ ID NO: 2047 and an AAV9K449R capsid. In some embodiments, the AAV particle comprises a viral genome comprising SEQ ID NO: 2047 and an AAVPHP.B capsid. In some embodiments, the AAV particle comprises a viral genome comprising SEQ ID NO: 2047 and an AAVPHP.N capsid. In some embodiments, the AAV particle comprises a viral genome comprising SEQ ID NO: 2047 and an AAV2 variant capsid. In some embodiments, the AAV particle comprises a viral genome comprising SEQ ID NO: 2047 and an AAV2/3 variant capsid. In some embodiments, the AAV particle comprises a viral genome comprising SEQ ID NO: 2047 and a capsid comprising SEQ ID NO: 1. In some embodiments, the AAV particle comprises a viral genome comprising SEQ ID NO: 2047 and a capsid encoded by a nucleic acid sequence comprising SEQ ID NO: 2. In some embodiments, the AAV particle comprises a viral genome comprising SEQ ID NO: 2047 and a capsid comprising SEQ ID NO: 4534. In some embodiments, the AAV particle comprises a viral genome comprising SEQ ID NO: 2047 and a capsid encoded by a nucleic acid sequence comprising SEQ ID NO: 3. In some embodiments, the AAV particle comprises a viral genome comprising SEQ ID NO: 2047 and a capsid comprising SEQ ID NO: 138. In some embodiments, the AAV particle comprises a viral genome comprising SEQ ID NO: 2047 and a capsid encoded by a nucleic acid sequence comprising SEQ ID NO: 137. In some embodiments, the AAV particle comprises a viral genome comprising SEQ ID NO: 2047 and a capsid comprising SEQ ID NO: 5. In some embodiments, the AAV particle comprises a viral genome comprising SEQ ID NO: 2047 and a capsid encoded by a nucleic acid sequence comprising SEQ ID NO: 6. In some embodiments, the AAV particle comprises a viral genome comprising SEQ ID NO: 2047 and a capsid comprising SEQ ID NO: 4. In some embodiments, the AAV particle comprises a viral genome comprising SEQ ID NO: 2047 and a capsid comprising SEQ ID NO: 11. In some embodiments, the AAV particle comprises a viral genome comprising SEQ ID NO: 2047 and a capsid comprising SEQ ID NO: 2679. In some embodiments, the AAV particle comprises a viral genome comprising SEQ ID NO: 2047 and a capsid comprising SEQ ID NO: 2809. In some embodiments, the AAV particle comprises a viral genome comprising SEQ ID NO: 2047 and a capsid comprising SEQ ID NO: 2871.

In some embodiments, the AAV particle comprises a viral genome comprising SEQ ID NO: 2048 and a VOY101 capsid. In some embodiments, the AAV particle comprises a viral genome comprising SEQ ID NO: 2048 and a VOY201 capsid. In some embodiments, the AAV particle comprises a viral genome comprising SEQ ID NO: 2048 and an AAV9 capsid. In some embodiments, the AAV particle comprises a viral genome comprising SEQ ID NO: 2048 and an AAV9K449R capsid. In some embodiments, the AAV particle comprises a viral genome comprising SEQ ID NO: 2048 and an AAVPHP.B capsid. In some embodiments, the AAV particle comprises a viral genome comprising SEQ ID NO: 2048 and an AAVPHP.N capsid. In some embodiments, the AAV particle comprises a viral genome comprising SEQ ID NO: 2048 and an AAV2 variant capsid. In some embodiments, the AAV particle comprises a viral genome comprising SEQ ID NO: 2048 and an AAV2/3 variant capsid. In some embodiments, the AAV particle comprises a viral genome comprising SEQ ID NO: 2048 and a capsid comprising SEQ ID NO: 1. In some embodiments, the AAV particle comprises a viral genome comprising SEQ ID NO: 2048 and a capsid encoded by a nucleic acid sequence comprising SEQ ID NO: 2. In some embodiments, the AAV particle comprises a viral genome comprising SEQ ID NO: 2048 and a capsid comprising SEQ ID NO: 4534. In some embodiments, the AAV particle comprises a viral genome comprising SEQ ID NO: 2048 and a capsid encoded by a nucleic acid sequence comprising SEQ ID NO: 3. In some embodiments, the AAV particle comprises a viral genome comprising SEQ ID NO: 2048 and a capsid comprising SEQ ID NO: 138. In some embodiments, the AAV particle comprises a viral genome comprising SEQ ID NO: 2048 and a capsid encoded by a nucleic acid sequence comprising SEQ ID NO: 137. In some embodiments, the AAV particle comprises a viral genome comprising SEQ ID NO: 2048 and a capsid comprising SEQ ID NO: 5. In some embodiments, the AAV particle comprises a viral genome comprising SEQ ID NO: 2048 and a capsid encoded by a nucleic acid sequence comprising SEQ ID NO: 6. In some embodiments, the AAV particle comprises a viral genome comprising SEQ ID NO: 2048 and a capsid comprising SEQ ID NO: 4. In some embodiments, the AAV particle comprises a viral genome comprising SEQ ID NO: 2048 and a capsid comprising SEQ ID NO: 11. In some embodiments, the AAV particle comprises a viral genome comprising SEQ ID NO: 2048 and a capsid comprising SEQ ID NO: 2679. In some embodiments, the AAV particle comprises a viral genome comprising SEQ ID NO: 2048 and a capsid comprising SEQ ID NO: 2809. In some embodiments, the AAV particle comprises a viral genome comprising SEQ ID NO: 2048 and a capsid comprising SEQ ID NO: 2871.

In some embodiments, the AAV particle comprises a viral genome comprising SEQ ID NO: 2049 and a VOY101 capsid. In some embodiments, the AAV particle comprises a viral genome comprising SEQ ID NO: 2049 and a VOY201 capsid. In some embodiments, the AAV particle comprises a viral genome comprising SEQ ID NO: 2049 and an AAV9 capsid. In some embodiments, the AAV particle comprises a viral genome comprising SEQ ID NO: 2049 and an AAV9K449R capsid. In some embodiments, the AAV particle comprises a viral genome comprising SEQ ID NO: 2049 and an AAVPHP.B capsid. In some embodiments, the AAV particle comprises a viral genome comprising SEQ ID NO: 2049 and an AAVPHP.N capsid. In some embodiments, the AAV particle comprises a viral genome comprising SEQ ID NO: 2049 and an AAV2 variant capsid. In some embodiments, the AAV particle comprises a viral genome comprising SEQ ID NO: 2049 and an AAV2/3 variant capsid. In some embodiments, the AAV particle comprises a viral genome comprising SEQ ID NO: 2049 and a capsid comprising SEQ ID NO: 1. In some embodiments, the AAV particle comprises a viral genome comprising SEQ ID NO: 2049 and a capsid encoded by a nucleic acid sequence comprising SEQ ID NO: 2. In some embodiments, the AAV particle comprises a viral genome comprising SEQ ID NO: 2049 and a capsid comprising SEQ ID NO: 4534. In some embodiments, the AAV particle comprises a viral genome comprising SEQ ID NO: 2049 and a capsid encoded by a nucleic acid sequence comprising SEQ ID NO: 3. In some embodiments, the AAV particle comprises a viral genome comprising SEQ ID NO: 2049 and a capsid comprising SEQ ID NO: 138. In some embodiments, the AAV particle comprises a viral genome comprising SEQ ID NO: 2049 and a capsid encoded by a nucleic acid sequence comprising SEQ ID NO: 137. In some embodiments, the AAV particle comprises a viral genome comprising SEQ ID NO: 2049 and a capsid comprising SEQ ID NO: 5. In some embodiments, the AAV particle comprises a viral genome comprising SEQ ID NO: 2049 and a capsid encoded by a nucleic acid sequence comprising SEQ ID NO: 6. In some embodiments, the AAV particle comprises a viral genome comprising SEQ ID NO: 2049 and a capsid comprising SEQ ID NO: 4. In some embodiments, the AAV particle comprises a viral genome comprising SEQ ID NO: 2049 and a capsid comprising SEQ ID NO: 11. In some embodiments, the AAV particle comprises a viral genome comprising SEQ ID NO: 2049 and a capsid comprising SEQ ID NO: 2679. In some embodiments, the AAV particle comprises a viral genome comprising SEQ ID NO: 2049 and a capsid comprising SEQ ID NO: 2809. In some embodiments, the AAV particle comprises a viral genome comprising SEQ ID NO: 2049 and a capsid comprising SEQ ID NO: 2871.

In some embodiments, the AAV particle comprises a viral genome comprising SEQ ID NO: 2050 and a VOY101 capsid. In some embodiments, the AAV particle comprises a viral genome comprising SEQ ID NO: 2050 and a VOY201 capsid. In some embodiments, the AAV particle comprises a viral genome comprising SEQ ID NO: 2050 and an AAV9 capsid. In some embodiments, the AAV particle comprises a viral genome comprising SEQ ID NO: 2050 and an AAV9K449R capsid. In some embodiments, the AAV particle comprises a viral genome comprising SEQ ID NO: 2050 and an AAVPHP.B capsid. In some embodiments, the AAV particle comprises a viral genome comprising SEQ ID NO: 2050 and an AAVPHP.N capsid. In some embodiments, the AAV particle comprises a viral genome comprising SEQ ID NO: 2050 and an AAV2 variant capsid. In some embodiments, the AAV particle comprises a viral genome comprising SEQ ID NO: 2050 and an AAV2/3 variant capsid. In some embodiments, the AAV particle comprises a viral genome comprising SEQ ID NO: 2050 and a capsid comprising SEQ ID NO: 1. In some embodiments, the AAV particle comprises a viral genome comprising SEQ ID NO: 2050 and a capsid encoded by a nucleic acid sequence comprising SEQ ID NO: 2. In some embodiments, the AAV particle comprises a viral genome comprising SEQ ID NO: 2050 and a capsid comprising SEQ ID NO: 4534. In some embodiments, the AAV particle comprises a viral genome comprising SEQ ID NO: 2050 and a capsid encoded by a nucleic acid sequence comprising SEQ ID NO: 3. In some embodiments, the AAV particle comprises a viral genome comprising SEQ ID NO: 2050 and a capsid comprising SEQ ID NO: 138. In some embodiments, the AAV particle comprises a viral genome comprising SEQ ID NO: 2050 and a capsid encoded by a nucleic acid sequence comprising SEQ ID NO: 137. In some embodiments, the AAV particle comprises a viral genome comprising SEQ ID NO: 2050 and a capsid comprising SEQ ID NO: 5. In some embodiments, the AAV particle comprises a viral genome comprising SEQ ID NO: 2050 and a capsid encoded by a nucleic acid sequence comprising SEQ ID NO: 6. In some embodiments, the AAV particle comprises a viral genome comprising SEQ ID NO: 2050 and a capsid comprising SEQ ID NO: 4. In some embodiments, the AAV particle comprises a viral genome comprising SEQ ID NO: 2050 and a capsid comprising SEQ ID NO: 11. In some embodiments, the AAV particle comprises a viral genome comprising SEQ ID NO: 2050 and a capsid comprising SEQ ID NO: 2679. In some embodiments, the AAV particle comprises a viral genome comprising SEQ ID NO: 2050 and a capsid comprising SEQ ID NO: 2809. In some embodiments, the AAV particle comprises a viral genome comprising SEQ ID NO: 2050 and a capsid comprising SEQ ID NO: 2871.

In some embodiments, the AAV particle comprises a viral genome comprising SEQ ID NO: 2051 and a VOY101 capsid. In some embodiments, the AAV particle comprises a viral genome comprising SEQ ID NO: 2051 and a VOY201 capsid. In some embodiments, the AAV particle comprises a viral genome comprising SEQ ID NO: 2051 and an AAV9 capsid. In some embodiments, the AAV particle comprises a viral genome comprising SEQ ID NO: 2051 and an AAV9K449R capsid. In some embodiments, the AAV particle comprises a viral genome comprising SEQ ID NO: 2051 and an AAVPHP.B capsid. In some embodiments, the AAV particle comprises a viral genome comprising SEQ ID NO: 2051 and an AAVPHP.N capsid. In some embodiments, the AAV particle comprises a viral genome comprising SEQ ID NO: 2051 and an AAV2 variant capsid. In some embodiments, the AAV particle comprises a viral genome comprising SEQ ID NO: 2051 and an AAV2/3 variant capsid. In some embodiments, the AAV particle comprises a viral genome comprising SEQ ID NO: 2051 and a capsid comprising SEQ ID NO: 1. In some embodiments, the AAV particle comprises a viral genome comprising SEQ ID NO: 2051 and a capsid encoded by a nucleic acid sequence comprising SEQ ID NO: 2. In some embodiments, the AAV particle comprises a viral genome comprising SEQ ID NO: 2051 and a capsid comprising SEQ ID NO: 4534. In some embodiments, the AAV particle comprises a viral genome comprising SEQ ID NO: 2051 and a capsid encoded by a nucleic acid sequence comprising SEQ ID NO: 3. In some embodiments, the AAV particle comprises a viral genome comprising SEQ ID NO: 2051 and a capsid comprising SEQ ID NO: 138. In some embodiments, the AAV particle comprises a viral genome comprising SEQ ID NO: 2051 and a capsid encoded by a nucleic acid sequence comprising SEQ ID NO: 137. In some embodiments, the AAV particle comprises a viral genome comprising SEQ ID NO: 2051 and a capsid comprising SEQ ID NO: 5. In some embodiments, the AAV particle comprises a viral genome comprising SEQ ID NO: 2051 and a capsid encoded by a nucleic acid sequence comprising SEQ ID NO: 6. In some embodiments, the AAV particle comprises a viral genome comprising SEQ ID NO: 2051 and a capsid comprising SEQ ID NO: 4. In some embodiments, the AAV particle comprises a viral genome comprising SEQ ID NO: 2051 and a capsid comprising SEQ ID NO: 11. In some embodiments, the AAV particle comprises a viral genome comprising SEQ ID NO: 2051 and a capsid comprising SEQ ID NO: 2679. In some embodiments, the AAV particle comprises a viral genome comprising SEQ ID NO: 2051 and a capsid comprising SEQ ID NO: 2809. In some embodiments, the AAV particle comprises a viral genome comprising SEQ ID NO: 2051 and a capsid comprising SEQ ID NO: 2871.

In some embodiments, the AAV particle comprises a viral genome comprising SEQ ID NO: 2052 and a VOY101 capsid. In some embodiments, the AAV particle comprises a viral genome comprising SEQ ID NO: 2052 and a VOY201 capsid. In some embodiments, the AAV particle comprises a viral genome comprising SEQ ID NO: 2052 and an AAV9 capsid. In some embodiments, the AAV particle comprises a viral genome comprising SEQ ID NO: 2052 and an AAV9K449R capsid. In some embodiments, the AAV particle comprises a viral genome comprising SEQ ID NO: 2052 and an AAVPHP.B capsid. In some embodiments, the AAV particle comprises a viral genome comprising SEQ ID NO: 2052 and an AAVPHP.N capsid. In some embodiments, the AAV particle comprises a viral genome comprising SEQ ID NO: 2052 and an AAV2 variant capsid. In some embodiments, the AAV particle comprises a viral genome comprising SEQ ID NO: 2052 and an AAV2/3 variant capsid. In some embodiments, the AAV particle comprises a viral genome comprising SEQ ID NO: 2052 and a capsid comprising SEQ ID NO: 1. In some embodiments, the AAV particle comprises a viral genome comprising SEQ ID NO: 2052 and a capsid encoded by a nucleic acid sequence comprising SEQ ID NO: 2. In some embodiments, the AAV particle comprises a viral genome comprising SEQ ID NO: 2052 and a capsid comprising SEQ ID NO: 4534. In some embodiments, the AAV particle comprises a viral genome comprising SEQ ID NO: 2052 and a capsid encoded by a nucleic acid sequence comprising SEQ ID NO: 3. In some embodiments, the AAV particle comprises a viral genome comprising SEQ ID NO: 2052 and a capsid comprising SEQ ID NO: 138. In some embodiments, the AAV particle comprises a viral genome comprising SEQ ID NO: 2052 and a capsid encoded by a nucleic acid sequence comprising SEQ ID NO: 137. In some embodiments, the AAV particle comprises a viral genome comprising SEQ ID NO: 2052 and a capsid comprising SEQ ID NO: 5. In some embodiments, the AAV particle comprises a viral genome comprising SEQ ID NO: 2052 and a capsid encoded by a nucleic acid sequence comprising SEQ ID NO: 6. In some embodiments, the AAV particle comprises a viral genome comprising SEQ ID NO: 2052 and a capsid comprising SEQ ID NO: 4. In some embodiments, the AAV particle comprises a viral genome comprising SEQ ID NO: 2052 and a capsid comprising SEQ ID NO: 11. In some embodiments, the AAV particle comprises a viral genome comprising SEQ ID NO: 2052 and a capsid comprising SEQ ID NO: 2679. In some embodiments, the AAV particle comprises a viral genome comprising SEQ ID NO: 2052 and a capsid comprising SEQ ID NO: 2809. In some embodiments, the AAV particle comprises a viral genome comprising SEQ ID NO: 2052 and a capsid comprising SEQ ID NO: 2871.

In some embodiments, the AAV particle comprises a viral genome comprising SEQ ID NO: 2053 and a VOY101 capsid. In some embodiments, the AAV particle comprises a viral genome comprising SEQ ID NO: 2053 and a VOY201 capsid. In some embodiments, the AAV particle comprises a viral genome comprising SEQ ID NO: 2053 and an AAV9 capsid. In some embodiments, the AAV particle comprises a viral genome comprising SEQ ID NO: 2053 and an AAV9K449R capsid. In some embodiments, the AAV particle comprises a viral genome comprising SEQ ID NO: 2053 and an AAVPHP.B capsid. In some embodiments, the AAV particle comprises a viral genome comprising SEQ ID NO: 2053 and an AAVPHP.N capsid. In some embodiments, the AAV particle comprises a viral genome comprising SEQ ID NO: 2053 and an AAV2 variant capsid. In some embodiments, the AAV particle comprises a viral genome comprising SEQ ID NO: 2053 and an AAV2/3 variant capsid. In some embodiments, the AAV particle comprises a viral genome comprising SEQ ID NO: 2053 and a capsid comprising SEQ ID NO: 1. In some embodiments, the AAV particle comprises a viral genome comprising SEQ ID NO: 2053 and a capsid encoded by a nucleic acid sequence comprising SEQ ID NO: 2. In some embodiments, the AAV particle comprises a viral genome comprising SEQ ID NO: 2053 and a capsid comprising SEQ ID NO: 4534. In some embodiments, the AAV particle comprises a viral genome comprising SEQ ID NO: 2053 and a capsid encoded by a nucleic acid sequence comprising SEQ ID NO: 3. In some embodiments, the AAV particle comprises a viral genome comprising SEQ ID NO: 2053 and a capsid comprising SEQ ID NO: 138. In some embodiments, the AAV particle comprises a viral genome comprising SEQ ID NO: 2053 and a capsid encoded by a nucleic acid sequence comprising SEQ ID NO: 137. In some embodiments, the AAV particle comprises a viral genome comprising SEQ ID NO: 2053 and a capsid comprising SEQ ID NO: 5. In some embodiments, the AAV particle comprises a viral genome comprising SEQ ID NO: 2053 and a capsid encoded by a nucleic acid sequence comprising SEQ ID NO: 6. In some embodiments, the AAV particle comprises a viral genome comprising SEQ ID NO: 2053 and a capsid comprising SEQ ID NO: 4. In some embodiments, the AAV particle comprises a viral genome comprising SEQ ID NO: 2053 and a capsid comprising SEQ ID NO: 11. In some embodiments, the AAV particle comprises a viral genome comprising SEQ ID NO: 2053 and a capsid comprising SEQ ID NO: 2679. In some embodiments, the AAV particle comprises a viral genome comprising SEQ ID NO: 2053 and a capsid comprising SEQ ID NO: 2809. In some embodiments, the AAV particle comprises a viral genome comprising SEQ ID NO: 2053 and a capsid comprising SEQ ID NO: 2871.

In some embodiments, the AAV particle comprises a viral genome comprising SEQ ID NO: 2054 and a VOY101 capsid. In some embodiments, the AAV particle comprises a viral genome comprising SEQ ID NO: 2054 and a VOY201 capsid. In some embodiments, the AAV particle comprises a viral genome comprising SEQ ID NO: 2054 and an AAV9 capsid. In some embodiments, the AAV particle comprises a viral genome comprising SEQ ID NO: 2054 and an AAV9K449R capsid. In some embodiments, the AAV particle comprises a viral genome comprising SEQ ID NO: 2054 and an AAVPHP.B capsid. In some embodiments, the AAV particle comprises a viral genome comprising SEQ ID NO: 2054 and an AAVPHP.N capsid. In some embodiments, the AAV particle comprises a viral genome comprising SEQ ID NO: 2054 and an AAV2 variant capsid. In some embodiments, the AAV particle comprises a viral genome comprising SEQ ID NO: 2054 and an AAV2/3 variant capsid. In some embodiments, the AAV particle comprises a viral genome comprising SEQ ID NO: 2054 and a capsid comprising SEQ ID NO: 1. In some embodiments, the AAV particle comprises a viral genome comprising SEQ ID NO: 2054 and a capsid encoded by a nucleic acid sequence comprising SEQ ID NO: 2. In some embodiments, the AAV particle comprises a viral genome comprising SEQ ID NO: 2054 and a capsid comprising SEQ ID NO: 4534. In some embodiments, the AAV particle comprises a viral genome comprising SEQ ID NO: 2054 and a capsid encoded by a nucleic acid sequence comprising SEQ ID NO: 3. In some embodiments, the AAV particle comprises a viral genome comprising SEQ ID NO: 2054 and a capsid comprising SEQ ID NO: 138. In some embodiments, the AAV particle comprises a viral genome comprising SEQ ID NO: 2054 and a capsid encoded by a nucleic acid sequence comprising SEQ ID NO: 137. In some embodiments, the AAV particle comprises a viral genome comprising SEQ ID NO: 2054 and a capsid comprising SEQ ID NO: 5. In some embodiments, the AAV particle comprises a viral genome comprising SEQ ID NO: 2054 and a capsid encoded by a nucleic acid sequence comprising SEQ ID NO: 6. In some embodiments, the AAV particle comprises a viral genome comprising SEQ ID NO: 2054 and a capsid comprising SEQ ID NO: 4. In some embodiments, the AAV particle comprises a viral genome comprising SEQ ID NO: 2054 and a capsid comprising SEQ ID NO: 11. In some embodiments, the AAV particle comprises a viral genome comprising SEQ ID NO: 2054 and a capsid comprising SEQ ID NO: 2679. In some embodiments, the AAV particle comprises a viral genome comprising SEQ ID NO: 2054 and a capsid comprising SEQ ID NO: 2809. In some embodiments, the AAV particle comprises a viral genome comprising SEQ ID NO: 2054 and a capsid comprising SEQ ID NO: 2871.

In some embodiments, the AAV particle comprises a viral genome comprising SEQ ID NO: 2055 and a VOY101 capsid. In some embodiments, the AAV particle comprises a viral genome comprising SEQ ID NO: 2055 and a VOY201 capsid. In some embodiments, the AAV particle comprises a viral genome comprising SEQ ID NO: 2055 and an AAV9 capsid. In some embodiments, the AAV particle comprises a viral genome comprising SEQ ID NO: 2055 and an AAV9K449R capsid. In some embodiments, the AAV particle comprises a viral genome comprising SEQ ID NO: 2055 and an AAVPHP.B capsid. In some embodiments, the AAV particle comprises a viral genome comprising SEQ ID NO: 2055 and an AAVPHP.N capsid. In some embodiments, the AAV particle comprises a viral genome comprising SEQ ID NO: 2055 and an AAV2 variant capsid. In some embodiments, the AAV particle comprises a viral genome comprising SEQ ID NO: 2055 and an AAV2/3 variant capsid. In some embodiments, the AAV particle comprises a viral genome comprising SEQ ID NO: 2055 and a capsid comprising SEQ ID NO: 1. In some embodiments, the AAV particle comprises a viral genome comprising SEQ ID NO: 2055 and a capsid encoded by a nucleic acid sequence comprising SEQ ID NO: 2. In some embodiments, the AAV particle comprises a viral genome comprising SEQ ID NO: 2055 and a capsid comprising SEQ ID NO: 4534. In some embodiments, the AAV particle comprises a viral genome comprising SEQ ID NO: 2055 and a capsid encoded by a nucleic acid sequence comprising SEQ ID NO: 3. In some embodiments, the AAV particle comprises a viral genome comprising SEQ ID NO: 2055 and a capsid comprising SEQ ID NO: 138. In some embodiments, the AAV particle comprises a viral genome comprising SEQ ID NO: 2055 and a capsid encoded by a nucleic acid sequence comprising SEQ ID NO: 137. In some embodiments, the AAV particle comprises a viral genome comprising SEQ ID NO: 2055 and a capsid comprising SEQ ID NO: 5. In some embodiments, the AAV particle comprises a viral genome comprising SEQ ID NO: 2055 and a capsid encoded by a nucleic acid sequence comprising SEQ ID NO: 6. In some embodiments, the AAV particle comprises a viral genome comprising SEQ ID NO: 2055 and a capsid comprising SEQ ID NO: 4. In some embodiments, the AAV particle comprises a viral genome comprising SEQ ID NO: 2055 and a capsid comprising SEQ ID NO: 11. In some embodiments, the AAV particle comprises a viral genome comprising SEQ ID NO: 2055 and a capsid comprising SEQ ID NO: 2679. In some embodiments, the AAV particle comprises a viral genome comprising SEQ ID NO: 2055 and a capsid comprising SEQ ID NO: 2809. In some embodiments, the AAV particle comprises a viral genome comprising SEQ ID NO: 2055 and a capsid comprising SEQ ID NO: 2871.

In some embodiments, the AAV particle comprises a viral genome comprising SEQ ID NO: 2056 and a VOY101 capsid. In some embodiments, the AAV particle comprises a viral genome comprising SEQ ID NO: 2056 and a VOY201 capsid. In some embodiments, the AAV particle comprises a viral genome comprising SEQ ID NO: 2056 and an AAV9 capsid. In some embodiments, the AAV particle comprises a viral genome comprising SEQ ID NO: 2056 and an AAV9K449R capsid. In some embodiments, the AAV particle comprises a viral genome comprising SEQ ID NO: 2056 and an AAVPHP.B capsid. In some embodiments, the AAV particle comprises a viral genome comprising SEQ ID NO: 2056 and an AAVPHP.N capsid. In some embodiments, the AAV particle comprises a viral genome comprising SEQ ID NO: 2056 and an AAV2 variant capsid. In some embodiments, the AAV particle comprises a viral genome comprising SEQ ID NO: 2056 and an AAV2/3 variant capsid. In some embodiments, the AAV particle comprises a viral genome comprising SEQ ID NO: 2056 and a capsid comprising SEQ ID NO: 1. In some embodiments, the AAV particle comprises a viral genome comprising SEQ ID NO: 2056 and a capsid encoded by a nucleic acid sequence comprising SEQ ID NO: 2. In some embodiments, the AAV particle comprises a viral genome comprising SEQ ID NO: 2056 and a capsid comprising SEQ ID NO: 4534. In some embodiments, the AAV particle comprises a viral genome comprising SEQ ID NO: 2056 and a capsid encoded by a nucleic acid sequence comprising SEQ ID NO: 3. In some embodiments, the AAV particle comprises a viral genome comprising SEQ ID NO: 2056 and a capsid comprising SEQ ID NO: 138. In some embodiments, the AAV particle comprises a viral genome comprising SEQ ID NO: 2056 and a capsid encoded by a nucleic acid sequence comprising SEQ ID NO: 137. In some embodiments, the AAV particle comprises a viral genome comprising SEQ ID NO: 2056 and a capsid comprising SEQ ID NO: 5. In some embodiments, the AAV particle comprises a viral genome comprising SEQ ID NO: 2056 and a capsid encoded by a nucleic acid sequence comprising SEQ ID NO: 6. In some embodiments, the AAV particle comprises a viral genome comprising SEQ ID NO: 2056 and a capsid comprising SEQ ID NO: 4. In some embodiments, the AAV particle comprises a viral genome comprising SEQ ID NO: 2056 and a capsid comprising SEQ ID NO: 11. In some embodiments, the AAV particle comprises a viral genome comprising SEQ ID NO: 2056 and a capsid comprising SEQ ID NO: 2679. In some embodiments, the AAV particle comprises a viral genome comprising SEQ ID NO: 2056 and a capsid comprising SEQ ID NO: 2809. In some embodiments, the AAV particle comprises a viral genome comprising SEQ ID NO: 2056 and a capsid comprising SEQ ID NO: 2871.

In some embodiments, the AAV particle comprises a viral genome comprising SEQ ID NO: 2057 and a VOY101 capsid. In some embodiments, the AAV particle comprises a viral genome comprising SEQ ID NO: 2057 and a VOY201 capsid. In some embodiments, the AAV particle comprises a viral genome comprising SEQ ID NO: 2057 and an AAV9 capsid. In some embodiments, the AAV particle comprises a viral genome comprising SEQ ID NO: 2057 and an AAV9K449R capsid. In some embodiments, the AAV particle comprises a viral genome comprising SEQ ID NO: 2057 and an AAVPHP.B capsid. In some embodiments, the AAV particle comprises a viral genome comprising SEQ ID NO: 2057 and an AAVPHP.N capsid. In some embodiments, the AAV particle comprises a viral genome comprising SEQ ID NO: 2057 and an AAV2 variant capsid. In some embodiments, the AAV particle comprises a viral genome comprising SEQ ID NO: 2057 and an AAV2/3 variant capsid. In some embodiments, the AAV particle comprises a viral genome comprising SEQ ID NO: 2057 and a capsid comprising SEQ ID NO: 1. In some embodiments, the AAV particle comprises a viral genome comprising SEQ ID NO: 2057 and a capsid encoded by a nucleic acid sequence comprising SEQ ID NO: 2. In some embodiments, the AAV particle comprises a viral genome comprising SEQ ID NO: 2057 and a capsid comprising SEQ ID NO: 4534. In some embodiments, the AAV particle comprises a viral genome comprising SEQ ID NO: 2057 and a capsid encoded by a nucleic acid sequence comprising SEQ ID NO: 3. In some embodiments, the AAV particle comprises a viral genome comprising SEQ ID NO: 2057 and a capsid comprising SEQ ID NO: 138. In some embodiments, the AAV particle comprises a viral genome comprising SEQ ID NO: 2057 and a capsid encoded by a nucleic acid sequence comprising SEQ ID NO: 137. In some embodiments, the AAV particle comprises a viral genome comprising SEQ ID NO: 2057 and a capsid comprising SEQ ID NO: 5. In some embodiments, the AAV particle comprises a viral genome comprising SEQ ID NO: 2057 and a capsid encoded by a nucleic acid sequence comprising SEQ ID NO: 6. In some embodiments, the AAV particle comprises a viral genome comprising SEQ ID NO: 2057 and a capsid comprising SEQ ID NO: 4. In some embodiments, the AAV particle comprises a viral genome comprising SEQ ID NO: 2057 and a capsid comprising SEQ ID NO: 11. In some embodiments, the AAV particle comprises a viral genome comprising SEQ ID NO: 2057 and a capsid comprising SEQ ID NO: 2679. In some embodiments, the AAV particle comprises a viral genome comprising SEQ ID NO: 2057 and a capsid comprising SEQ ID NO: 2809. In some embodiments, the AAV particle comprises a viral genome comprising SEQ ID NO: 2057 and a capsid comprising SEQ ID NO: 2871.

In some embodiments, the AAV particle comprises a viral genome comprising SEQ ID NO: 2058 and a VOY101 capsid. In some embodiments, the AAV particle comprises a viral genome comprising SEQ ID NO: 2058 and a VOY201 capsid. In some embodiments, the AAV particle comprises a viral genome comprising SEQ ID NO: 2058 and an AAV9 capsid. In some embodiments, the AAV particle comprises a viral genome comprising SEQ ID NO: 2058 and an AAV9K449R capsid. In some embodiments, the AAV particle comprises a viral genome comprising SEQ ID NO: 2058 and an AAVPHP.B capsid. In some embodiments, the AAV particle comprises a viral genome comprising SEQ ID NO: 2058 and an AAVPHP.N capsid. In some embodiments, the AAV particle comprises a viral genome comprising SEQ ID NO: 2058 and an AAV2 variant capsid. In some embodiments, the AAV particle comprises a viral genome comprising SEQ ID NO: 2058 and an AAV2/3 variant capsid. In some embodiments, the AAV particle comprises a viral genome comprising SEQ ID NO: 2058 and a capsid comprising SEQ ID NO: 1. In some embodiments, the AAV particle comprises a viral genome comprising SEQ ID NO: 2058 and a capsid encoded by a nucleic acid sequence comprising SEQ ID NO: 2. In some embodiments, the AAV particle comprises a viral genome comprising SEQ ID NO: 2058 and a capsid comprising SEQ ID NO: 4534. In some embodiments, the AAV particle comprises a viral genome comprising SEQ ID NO: 2058 and a capsid encoded by a nucleic acid sequence comprising SEQ ID NO: 3. In some embodiments, the AAV particle comprises a viral genome comprising SEQ ID NO: 2058 and a capsid comprising SEQ ID NO: 138. In some embodiments, the AAV particle comprises a viral genome comprising SEQ ID NO: 2058 and a capsid encoded by a nucleic acid sequence comprising SEQ ID NO: 137. In some embodiments, the AAV particle comprises a viral genome comprising SEQ ID NO: 2058 and a capsid comprising SEQ ID NO: 5. In some embodiments, the AAV particle comprises a viral genome comprising SEQ ID NO: 2058 and a capsid encoded by a nucleic acid sequence comprising SEQ ID NO: 6. In some embodiments, the AAV particle comprises a viral genome comprising SEQ ID NO: 2058 and a capsid comprising SEQ ID NO: 4. In some embodiments, the AAV particle comprises a viral genome comprising SEQ ID NO: 2058 and a capsid comprising SEQ ID NO: 11. In some embodiments, the AAV particle comprises a viral genome comprising SEQ ID NO: 2058 and a capsid comprising SEQ ID NO: 2679. In some embodiments, the AAV particle comprises a viral genome comprising SEQ ID NO: 2058 and a capsid comprising SEQ ID NO: 2809. In some embodiments, the AAV particle comprises a viral genome comprising SEQ ID NO: 2058 and a capsid comprising SEQ ID NO: 2871.

In some embodiments, the AAV particle comprises a viral genome comprising SEQ ID NO: 2059 and a VOY101 capsid. In some embodiments, the AAV particle comprises a viral genome comprising SEQ ID NO: 2059 and a VOY201 capsid. In some embodiments, the AAV particle comprises a viral genome comprising SEQ ID NO: 2059 and an AAV9 capsid. In some embodiments, the AAV particle comprises a viral genome comprising SEQ ID NO: 2059 and an AAV9K449R capsid. In some embodiments, the AAV particle comprises a viral genome comprising SEQ ID NO: 2059 and an AAVPHP.B capsid. In some embodiments, the AAV particle comprises a viral genome comprising SEQ ID NO: 2059 and an AAVPHP.N capsid. In some embodiments, the AAV particle comprises a viral genome comprising SEQ ID NO: 2059 and an AAV2 variant capsid. In some embodiments, the AAV particle comprises a viral genome comprising SEQ ID NO: 2059 and an AAV2/3 variant capsid. In some embodiments, the AAV particle comprises a viral genome comprising SEQ ID NO: 2059 and a capsid comprising SEQ ID NO: 1. In some embodiments, the AAV particle comprises a viral genome comprising SEQ ID NO: 2059 and a capsid encoded by a nucleic acid sequence comprising SEQ ID NO: 2. In some embodiments, the AAV particle comprises a viral genome comprising SEQ ID NO: 2059 and a capsid comprising SEQ ID NO: 4534. In some embodiments, the AAV particle comprises a viral genome comprising SEQ ID NO: 2059 and a capsid encoded by a nucleic acid sequence comprising SEQ ID NO: 3. In some embodiments, the AAV particle comprises a viral genome comprising SEQ ID NO: 2059 and a capsid comprising SEQ ID NO: 138. In some embodiments, the AAV particle comprises a viral genome comprising SEQ ID NO: 2059 and a capsid encoded by a nucleic acid sequence comprising SEQ ID NO: 137. In some embodiments, the AAV particle comprises a viral genome comprising SEQ ID NO: 2059 and a capsid comprising SEQ ID NO: 5. In some embodiments, the AAV particle comprises a viral genome comprising SEQ ID NO: 2059 and a capsid encoded by a nucleic acid sequence comprising SEQ ID NO: 6. In some embodiments, the AAV particle comprises a viral genome comprising SEQ ID NO: 2059 and a capsid comprising SEQ ID NO: 4. In some embodiments, the AAV particle comprises a viral genome comprising SEQ ID NO: 2059 and a capsid comprising SEQ ID NO: 11. In some embodiments, the AAV particle comprises a viral genome comprising SEQ ID NO: 2059 and a capsid comprising SEQ ID NO: 2679. In some embodiments, the AAV particle comprises a viral genome comprising SEQ ID NO: 2059 and a capsid comprising SEQ ID NO: 2809. In some embodiments, the AAV particle comprises a viral genome comprising SEQ ID NO: 2059 and a capsid comprising SEQ ID NO: 2871.

In some embodiments, the AAV particle comprises a viral genome comprising SEQ ID NO: 2060 and a VOY101 capsid. In some embodiments, the AAV particle comprises a viral genome comprising SEQ ID NO: 2060 and a VOY201 capsid. In some embodiments, the AAV particle comprises a viral genome comprising SEQ ID NO: 2060 and an AAV9 capsid. In some embodiments, the AAV particle comprises a viral genome comprising SEQ ID NO: 2060 and an AAV9K449R capsid. In some embodiments, the AAV particle comprises a viral genome comprising SEQ ID NO: 2060 and an AAVPHP.B capsid. In some embodiments, the AAV particle comprises a viral genome comprising SEQ ID NO: 2060 and an AAVPHP.N capsid. In some embodiments, the AAV particle comprises a viral genome comprising SEQ ID NO: 2060 and an AAV2 variant capsid. In some embodiments, the AAV particle comprises a viral genome comprising SEQ ID NO: 2060 and an AAV2/3 variant capsid. In some embodiments, the AAV particle comprises a viral genome comprising SEQ ID NO: 2060 and a capsid comprising SEQ ID NO: 1. In some embodiments, the AAV particle comprises a viral genome comprising SEQ ID NO: 2060 and a capsid encoded by a nucleic acid sequence comprising SEQ ID NO: 2. In some embodiments, the AAV particle comprises a viral genome comprising SEQ ID NO: 2060 and a capsid comprising SEQ ID NO: 4534. In some embodiments, the AAV particle comprises a viral genome comprising SEQ ID NO: 2060 and a capsid encoded by a nucleic acid sequence comprising SEQ ID NO: 3. In some embodiments, the AAV particle comprises a viral genome comprising SEQ ID NO: 2060 and a capsid comprising SEQ ID NO: 138. In some embodiments, the AAV particle comprises a viral genome comprising SEQ ID NO: 2060 and a capsid encoded by a nucleic acid sequence comprising SEQ ID NO: 137. In some embodiments, the AAV particle comprises a viral genome comprising SEQ ID NO: 2060 and a capsid comprising SEQ ID NO: 5. In some embodiments, the AAV particle comprises a viral genome comprising SEQ ID NO: 2060 and a capsid encoded by a nucleic acid sequence comprising SEQ ID NO: 6. In some embodiments, the AAV particle comprises a viral genome comprising SEQ ID NO: 2060 and a capsid comprising SEQ ID NO: 4. In some embodiments, the AAV particle comprises a viral genome comprising SEQ ID NO: 2060 and a capsid comprising SEQ ID NO: 11. In some embodiments, the AAV particle comprises a viral genome comprising SEQ ID NO: 2060 and a capsid comprising SEQ ID NO: 2679. In some embodiments, the AAV particle comprises a viral genome comprising SEQ ID NO: 2060 and a capsid comprising SEQ ID NO: 2809. In some embodiments, the AAV particle comprises a viral genome comprising SEQ ID NO: 2060 and a capsid comprising SEQ ID NO: 2871.

In some embodiments, the AAV particle comprises a viral genome comprising SEQ ID NO: 2061 and a VOY101 capsid. In some embodiments, the AAV particle comprises a viral genome comprising SEQ ID NO: 2061 and a VOY201 capsid. In some embodiments, the AAV particle comprises a viral genome comprising SEQ ID NO: 2061 and an AAV9 capsid. In some embodiments, the AAV particle comprises a viral genome comprising SEQ ID NO: 2061 and an AAV9K449R capsid. In some embodiments, the AAV particle comprises a viral genome comprising SEQ ID NO: 2061 and an AAVPHP.B capsid. In some embodiments, the AAV particle comprises a viral genome comprising SEQ ID NO: 2061 and an AAVPHP.N capsid. In some embodiments, the AAV particle comprises a viral genome comprising SEQ ID NO: 2061 and an AAV2 variant capsid. In some embodiments, the AAV particle comprises a viral genome comprising SEQ ID NO: 2061 and an AAV2/3 variant capsid. In some embodiments, the AAV particle comprises a viral genome comprising SEQ ID NO: 2061 and a capsid comprising SEQ ID NO: 1. In some embodiments, the AAV particle comprises a viral genome comprising SEQ ID NO: 2061 and a capsid encoded by a nucleic acid sequence comprising SEQ ID NO: 2. In some embodiments, the AAV particle comprises a viral genome comprising SEQ ID NO: 2061 and a capsid comprising SEQ ID NO: 4534. In some embodiments, the AAV particle comprises a viral genome comprising SEQ ID NO: 2061 and a capsid encoded by a nucleic acid sequence comprising SEQ ID NO: 3. In some embodiments, the AAV particle comprises a viral genome comprising SEQ ID NO: 2061 and a capsid comprising SEQ ID NO: 138. In some embodiments, the AAV particle comprises a viral genome comprising SEQ ID NO: 2061 and a capsid encoded by a nucleic acid sequence comprising SEQ ID NO: 137. In some embodiments, the AAV particle comprises a viral genome comprising SEQ ID NO: 2061 and a capsid comprising SEQ ID NO: 5. In some embodiments, the AAV particle comprises a viral genome comprising SEQ ID NO: 2061 and a capsid encoded by a nucleic acid sequence comprising SEQ ID NO: 6. In some embodiments, the AAV particle comprises a viral genome comprising SEQ ID NO: 2061 and a capsid comprising SEQ ID NO: 4. In some embodiments, the AAV particle comprises a viral genome comprising SEQ ID NO: 2061 and a capsid comprising SEQ ID NO: 11. In some embodiments, the AAV particle comprises a viral genome comprising SEQ ID NO: 2061 and a capsid comprising SEQ ID NO: 2679. In some embodiments, the AAV particle comprises a viral genome comprising SEQ ID NO: 2061 and a capsid comprising SEQ ID NO: 2809. In some embodiments, the AAV particle comprises a viral genome comprising SEQ ID NO: 2061 and a capsid comprising SEQ ID NO: 2871.

In some embodiments, the AAV particle comprises a viral genome comprising SEQ ID NO: 2062 and a VOY101 capsid. In some embodiments, the AAV particle comprises a viral genome comprising SEQ ID NO: 2062 and a VOY201 capsid. In some embodiments, the AAV particle comprises a viral genome comprising SEQ ID NO: 2062 and an AAV9 capsid. In some embodiments, the AAV particle comprises a viral genome comprising SEQ ID NO: 2062 and an AAV9K449R capsid. In some embodiments, the AAV particle comprises a viral genome comprising SEQ ID NO: 2062 and an AAVPHP.B capsid. In some embodiments, the AAV particle comprises a viral genome comprising SEQ ID NO: 2062 and an AAVPHP.N capsid. In some embodiments, the AAV particle comprises a viral genome comprising SEQ ID NO: 2062 and an AAV2 variant capsid. In some embodiments, the AAV particle comprises a viral genome comprising SEQ ID NO: 2062 and an AAV2/3 variant capsid. In some embodiments, the AAV particle comprises a viral genome comprising SEQ ID NO: 2062 and a capsid comprising SEQ ID NO: 1. In some embodiments, the AAV particle comprises a viral genome comprising SEQ ID NO: 2062 and a capsid encoded by a nucleic acid sequence comprising SEQ ID NO: 2. In some embodiments, the AAV particle comprises a viral genome comprising SEQ ID NO: 2062 and a capsid comprising SEQ ID NO: 4534. In some embodiments, the AAV particle comprises a viral genome comprising SEQ ID NO: 2062 and a capsid encoded by a nucleic acid sequence comprising SEQ ID NO: 3. In some embodiments, the AAV particle comprises a viral genome comprising SEQ ID NO: 2062 and a capsid comprising SEQ ID NO: 138. In some embodiments, the AAV particle comprises a viral genome comprising SEQ ID NO: 2062 and a capsid encoded by a nucleic acid sequence comprising SEQ ID NO: 137. In some embodiments, the AAV particle comprises a viral genome comprising SEQ ID NO: 2062 and a capsid comprising SEQ ID NO: 5. In some embodiments, the AAV particle comprises a viral genome comprising SEQ ID NO: 2062 and a capsid encoded by a nucleic acid sequence comprising SEQ ID NO: 6. In some embodiments, the AAV particle comprises a viral genome comprising SEQ ID NO: 2062 and a capsid comprising SEQ ID NO: 4. In some embodiments, the AAV particle comprises a viral genome comprising SEQ ID NO: 2062 and a capsid comprising SEQ ID NO: 11. In some embodiments, the AAV particle comprises a viral genome comprising SEQ ID NO: 2062 and a capsid comprising SEQ ID NO: 2679. In some embodiments, the AAV particle comprises a viral genome comprising SEQ ID NO: 2062 and a capsid comprising SEQ ID NO: 2809. In some embodiments, the AAV particle comprises a viral genome comprising SEQ ID NO: 2062 and a capsid comprising SEQ ID NO: 2871.

In some embodiments, the AAV particle comprises a viral genome comprising SEQ ID NO: 2063 and a VOY101 capsid. In some embodiments, the AAV particle comprises a viral genome comprising SEQ ID NO: 2063 and a VOY201 capsid. In some embodiments, the AAV particle comprises a viral genome comprising SEQ ID NO: 2063 and an AAV9 capsid. In some embodiments, the AAV particle comprises a viral genome comprising SEQ ID NO: 2063 and an AAV9K449R capsid. In some embodiments, the AAV particle comprises a viral genome comprising SEQ ID NO: 2063 and an AAVPHP.B capsid. In some embodiments, the AAV particle comprises a viral genome comprising SEQ ID NO: 2063 and an AAVPHP.N capsid. In some embodiments, the AAV particle comprises a viral genome comprising SEQ ID NO: 2063 and an AAV2 variant capsid. In some embodiments, the AAV particle comprises a viral genome comprising SEQ ID NO: 2063 and an AAV2/3 variant capsid. In some embodiments, the AAV particle comprises a viral genome comprising SEQ ID NO: 2063 and a capsid comprising SEQ ID NO: 1. In some embodiments, the AAV particle comprises a viral genome comprising SEQ ID NO: 2063 and a capsid encoded by a nucleic acid sequence comprising SEQ ID NO: 2. In some embodiments, the AAV particle comprises a viral genome comprising SEQ ID NO: 2063 and a capsid comprising SEQ ID NO: 4534. In some embodiments, the AAV particle comprises a viral genome comprising SEQ ID NO: 2063 and a capsid encoded by a nucleic acid sequence comprising SEQ ID NO: 3. In some embodiments, the AAV particle comprises a viral genome comprising SEQ ID NO: 2063 and a capsid comprising SEQ ID NO: 138. In some embodiments, the AAV particle comprises a viral genome comprising SEQ ID NO: 2063 and a capsid encoded by a nucleic acid sequence comprising SEQ ID NO: 137. In some embodiments, the AAV particle comprises a viral genome comprising SEQ ID NO: 2063 and a capsid comprising SEQ ID NO: 5. In some embodiments, the AAV particle comprises a viral genome comprising SEQ ID NO: 2063 and a capsid encoded by a nucleic acid sequence comprising SEQ ID NO: 6. In some embodiments, the AAV particle comprises a viral genome comprising SEQ ID NO: 2063 and a capsid comprising SEQ ID NO: 4. In some embodiments, the AAV particle comprises a viral genome comprising SEQ ID NO: 2063 and a capsid comprising SEQ ID NO: 11. In some embodiments, the AAV particle comprises a viral genome comprising SEQ ID NO: 2063 and a capsid comprising SEQ ID NO: 2679. In some embodiments, the AAV particle comprises a viral genome comprising SEQ ID NO: 2063 and a capsid comprising SEQ ID NO: 2809. In some embodiments, the AAV particle comprises a viral genome comprising SEQ ID NO: 2063 and a capsid comprising SEQ ID NO: 2871.

In some embodiments, the AAV particle comprises a viral genome comprising SEQ ID NO: 2064 and a VOY101 capsid. In some embodiments, the AAV particle comprises a viral genome comprising SEQ ID NO: 2064 and a VOY201 capsid. In some embodiments, the AAV particle comprises a viral genome comprising SEQ ID NO: 2064 and an AAV9 capsid. In some embodiments, the AAV particle comprises a viral genome comprising SEQ ID NO: 2064 and an AAV9K449R capsid. In some embodiments, the AAV particle comprises a viral genome comprising SEQ ID NO: 2064 and an AAVPHP.B capsid. In some embodiments, the AAV particle comprises a viral genome comprising SEQ ID NO: 2064 and an AAVPHP.N capsid. In some embodiments, the AAV particle comprises a viral genome comprising SEQ ID NO: 2064 and an AAV2 variant capsid. In some embodiments, the AAV particle comprises a viral genome comprising SEQ ID NO: 2064 and an AAV2/3 variant capsid. In some embodiments, the AAV particle comprises a viral genome comprising SEQ ID NO: 2064 and a capsid comprising SEQ ID NO: 1. In some embodiments, the AAV particle comprises a viral genome comprising SEQ ID NO: 2064 and a capsid encoded by a nucleic acid sequence comprising SEQ ID NO: 2. In some embodiments, the AAV particle comprises a viral genome comprising SEQ ID NO: 2064 and a capsid comprising SEQ ID NO: 4534. In some embodiments, the AAV particle comprises a viral genome comprising SEQ ID NO: 2064 and a capsid encoded by a nucleic acid sequence comprising SEQ ID NO: 3. In some embodiments, the AAV particle comprises a viral genome comprising SEQ ID NO: 2064 and a capsid comprising SEQ ID NO: 138. In some embodiments, the AAV particle comprises a viral genome comprising SEQ ID NO: 2064 and a capsid encoded by a nucleic acid sequence comprising SEQ ID NO: 137. In some embodiments, the AAV particle comprises a viral genome comprising SEQ ID NO: 2064 and a capsid comprising SEQ ID NO: 5. In some embodiments, the AAV particle comprises a viral genome comprising SEQ ID NO: 2064 and a capsid encoded by a nucleic acid sequence comprising SEQ ID NO: 6. In some embodiments, the AAV particle comprises a viral genome comprising SEQ ID NO: 2064 and a capsid comprising SEQ ID NO: 4. In some embodiments, the AAV particle comprises a viral genome comprising SEQ ID NO: 2064 and a capsid comprising SEQ ID NO: 11. In some embodiments, the AAV particle comprises a viral genome comprising SEQ ID NO: 2064 and a capsid comprising SEQ ID NO: 2679. In some embodiments, the AAV particle comprises a viral genome comprising SEQ ID NO: 2064 and a capsid comprising SEQ ID NO: 2809. In some embodiments, the AAV particle comprises a viral genome comprising SEQ ID NO: 2064 and a capsid comprising SEQ ID NO: 2871.

In some embodiments, the AAV particle comprises a viral genome comprising SEQ ID NO: 2065 and a VOY101 capsid. In some embodiments, the AAV particle comprises a viral genome comprising SEQ ID NO: 2065 and a VOY201 capsid. In some embodiments, the AAV particle comprises a viral genome comprising SEQ ID NO: 2065 and an AAV9 capsid. In some embodiments, the AAV particle comprises a viral genome comprising SEQ ID NO: 2065 and an AAV9K449R capsid. In some embodiments, the AAV particle comprises a viral genome comprising SEQ ID NO: 2065 and an AAVPHP.B capsid. In some embodiments, the AAV particle comprises a viral genome comprising SEQ ID NO: 2065 and an AAVPHP.N capsid. In some embodiments, the AAV particle comprises a viral genome comprising SEQ ID NO: 2065 and an AAV2 variant capsid. In some embodiments, the AAV particle comprises a viral genome comprising SEQ ID NO: 2065 and an AAV2/3 variant capsid. In some embodiments, the AAV particle comprises a viral genome comprising SEQ ID NO: 2065 and a capsid comprising SEQ ID NO: 1. In some embodiments, the AAV particle comprises a viral genome comprising SEQ ID NO: 2065 and a capsid encoded by a nucleic acid sequence comprising SEQ ID NO: 2. In some embodiments, the AAV particle comprises a viral genome comprising SEQ ID NO: 2065 and a capsid comprising SEQ ID NO: 4534. In some embodiments, the AAV particle comprises a viral genome comprising SEQ ID NO: 2065 and a capsid encoded by a nucleic acid sequence comprising SEQ ID NO: 3. In some embodiments, the AAV particle comprises a viral genome comprising SEQ ID NO: 2065 and a capsid comprising SEQ ID NO: 138. In some embodiments, the AAV particle comprises a viral genome comprising SEQ ID NO: 2065 and a capsid encoded by a nucleic acid sequence comprising SEQ ID NO: 137. In some embodiments, the AAV particle comprises a viral genome comprising SEQ ID NO: 2065 and a capsid comprising SEQ ID NO: 5. In some embodiments, the AAV particle comprises a viral genome comprising SEQ ID NO: 2065 and a capsid encoded by a nucleic acid sequence comprising SEQ ID NO: 6. In some embodiments, the AAV particle comprises a viral genome comprising SEQ ID NO: 2065 and a capsid comprising SEQ ID NO: 4. In some embodiments, the AAV particle comprises a viral genome comprising SEQ ID NO: 2065 and a capsid comprising SEQ ID NO: 11. In some embodiments, the AAV particle comprises a viral genome comprising SEQ ID NO: 2065 and a capsid comprising SEQ ID NO: 2679. In some embodiments, the AAV particle comprises a viral genome comprising SEQ ID NO: 2065 and a capsid comprising SEQ ID NO: 2809. In some embodiments, the AAV particle comprises a viral genome comprising SEQ ID NO: 2065 and a capsid comprising SEQ ID NO: 2871.

In some embodiments, the AAV particle comprises a viral genome comprising SEQ ID NO: 2066 and a VOY101 capsid. In some embodiments, the AAV particle comprises a viral genome comprising SEQ ID NO: 2066 and a VOY201 capsid. In some embodiments, the AAV particle comprises a viral genome comprising SEQ ID NO: 2066 and an AAV9 capsid. In some embodiments, the AAV particle comprises a viral genome comprising SEQ ID NO: 2066 and an AAV9K449R capsid. In some embodiments, the AAV particle comprises a viral genome comprising SEQ ID NO: 2066 and an AAVPHP.B capsid. In some embodiments, the AAV particle comprises a viral genome comprising SEQ ID NO: 2066 and an AAVPHP.N capsid. In some embodiments, the AAV particle comprises a viral genome comprising SEQ ID NO: 2066 and an AAV2 variant capsid. In some embodiments, the AAV particle comprises a viral genome comprising SEQ ID NO: 2066 and an AAV2/3 variant capsid. In some embodiments, the AAV particle comprises a viral genome comprising SEQ ID NO: 2066 and a capsid comprising SEQ ID NO: 1. In some embodiments, the AAV particle comprises a viral genome comprising SEQ ID NO: 2066 and a capsid encoded by a nucleic acid sequence comprising SEQ ID NO: 2. In some embodiments, the AAV particle comprises a viral genome comprising SEQ ID NO: 2066 and a capsid comprising SEQ ID NO: 4534. In some embodiments, the AAV particle comprises a viral genome comprising SEQ ID NO: 2066 and a capsid encoded by a nucleic acid sequence comprising SEQ ID NO: 3. In some embodiments, the AAV particle comprises a viral genome comprising SEQ ID NO: 2066 and a capsid comprising SEQ ID NO: 138. In some embodiments, the AAV particle comprises a viral genome comprising SEQ ID NO: 2066 and a capsid encoded by a nucleic acid sequence comprising SEQ ID NO: 137. In some embodiments, the AAV particle comprises a viral genome comprising SEQ ID NO: 2066 and a capsid comprising SEQ ID NO: 5. In some embodiments, the AAV particle comprises a viral genome comprising SEQ ID NO: 2066 and a capsid encoded by a nucleic acid sequence comprising SEQ ID NO: 6. In some embodiments, the AAV particle comprises a viral genome comprising SEQ ID NO: 2066 and a capsid comprising SEQ ID NO: 4. In some embodiments, the AAV particle comprises a viral genome comprising SEQ ID NO: 2066 and a capsid comprising SEQ ID NO: 11. In some embodiments, the AAV particle comprises a viral genome comprising SEQ ID NO: 2066 and a capsid comprising SEQ ID NO: 2679. In some embodiments, the AAV particle comprises a viral genome comprising SEQ ID NO: 2066 and a capsid comprising SEQ ID NO: 2809. In some embodiments, the AAV particle comprises a viral genome comprising SEQ ID NO: 2066 and a capsid comprising SEQ ID NO: 2871.

In some embodiments, the AAV particle comprises a viral genome comprising SEQ ID NO: 2067 and a VOY101 capsid. In some embodiments, the AAV particle comprises a viral genome comprising SEQ ID NO: 2067 and a VOY201 capsid. In some embodiments, the AAV particle comprises a viral genome comprising SEQ ID NO: 2067 and an AAV9 capsid. In some embodiments, the AAV particle comprises a viral genome comprising SEQ ID NO: 2067 and an AAV9K449R capsid. In some embodiments, the AAV particle comprises a viral genome comprising SEQ ID NO: 2067 and an AAVPHP.B capsid. In some embodiments, the AAV particle comprises a viral genome comprising SEQ ID NO: 2067 and an AAVPHP.N capsid. In some embodiments, the AAV particle comprises a viral genome comprising SEQ ID NO: 2067 and an AAV2 variant capsid. In some embodiments, the AAV particle comprises a viral genome comprising SEQ ID NO: 2067 and an AAV2/3 variant capsid. In some embodiments, the AAV particle comprises a viral genome comprising SEQ ID NO: 2067 and a capsid comprising SEQ ID NO: 1. In some embodiments, the AAV particle comprises a viral genome comprising SEQ ID NO: 2067 and a capsid encoded by a nucleic acid sequence comprising SEQ ID NO: 2. In some embodiments, the AAV particle comprises a viral genome comprising SEQ ID NO: 2067 and a capsid comprising SEQ ID NO: 4534. In some embodiments, the AAV particle comprises a viral genome comprising SEQ ID NO: 2067 and a capsid encoded by a nucleic acid sequence comprising SEQ ID NO: 3. In some embodiments, the AAV particle comprises a viral genome comprising SEQ ID NO: 2067 and a capsid comprising SEQ ID NO: 138. In some embodiments, the AAV particle comprises a viral genome comprising SEQ ID NO: 2067 and a capsid encoded by a nucleic acid sequence comprising SEQ ID NO: 137. In some embodiments, the AAV particle comprises a viral genome comprising SEQ ID NO: 2067 and a capsid comprising SEQ ID NO: 5. In some embodiments, the AAV particle comprises a viral genome comprising SEQ ID NO: 2067 and a capsid encoded by a nucleic acid sequence comprising SEQ ID NO: 6. In some embodiments, the AAV particle comprises a viral genome comprising SEQ ID NO: 2067 and a capsid comprising SEQ ID NO: 4. In some embodiments, the AAV particle comprises a viral genome comprising SEQ ID NO: 2067 and a capsid comprising SEQ ID NO: 11. In some embodiments, the AAV particle comprises a viral genome comprising SEQ ID NO: 2067 and a capsid comprising SEQ ID NO: 2679. In some embodiments, the AAV particle comprises a viral genome comprising SEQ ID NO: 2067 and a capsid comprising SEQ ID NO: 2809. In some embodiments, the AAV particle comprises a viral genome comprising SEQ ID NO: 2067 and a capsid comprising SEQ ID NO: 2871.

In some embodiments, the AAV particle comprises a viral genome comprising SEQ ID NO: 2068 and a VOY101 capsid. In some embodiments, the AAV particle comprises a viral genome comprising SEQ ID NO: 2068 and a VOY201 capsid. In some embodiments, the AAV particle comprises a viral genome comprising SEQ ID NO: 2068 and an AAV9 capsid. In some embodiments, the AAV particle comprises a viral genome comprising SEQ ID NO: 2068 and an AAV9K449R capsid. In some embodiments, the AAV particle comprises a viral genome comprising SEQ ID NO: 2068 and an AAVPHP.B capsid. In some embodiments, the AAV particle comprises a viral genome comprising SEQ ID NO: 2068 and an AAVPHP.N capsid. In some embodiments, the AAV particle comprises a viral genome comprising SEQ ID NO: 2068 and an AAV2 variant capsid. In some embodiments, the AAV particle comprises a viral genome comprising SEQ ID NO: 2068 and an AAV2/3 variant capsid. In some embodiments, the AAV particle comprises a viral genome comprising SEQ ID NO: 2068 and a capsid comprising SEQ ID NO: 1. In some embodiments, the AAV particle comprises a viral genome comprising SEQ ID NO: 2068 and a capsid encoded by a nucleic acid sequence comprising SEQ ID NO: 2. In some embodiments, the AAV particle comprises a viral genome comprising SEQ ID NO: 2068 and a capsid comprising SEQ ID NO: 4534. In some embodiments, the AAV particle comprises a viral genome comprising SEQ ID NO: 2068 and a capsid encoded by a nucleic acid sequence comprising SEQ ID NO: 3. In some embodiments, the AAV particle comprises a viral genome comprising SEQ ID NO: 2068 and a capsid comprising SEQ ID NO: 138. In some embodiments, the AAV particle comprises a viral genome comprising SEQ ID NO: 2068 and a capsid encoded by a nucleic acid sequence comprising SEQ ID NO: 137. In some embodiments, the AAV particle comprises a viral genome comprising SEQ ID NO: 2068 and a capsid comprising SEQ ID NO: 5. In some embodiments, the AAV particle comprises a viral genome comprising SEQ ID NO: 2068 and a capsid encoded by a nucleic acid sequence comprising SEQ ID NO: 6. In some embodiments, the AAV particle comprises a viral genome comprising SEQ ID NO: 2068 and a capsid comprising SEQ ID NO: 4. In some embodiments, the AAV particle comprises a viral genome comprising SEQ ID NO: 2068 and a capsid comprising SEQ ID NO: 11. In some embodiments, the AAV particle comprises a viral genome comprising SEQ ID NO: 2068 and a capsid comprising SEQ ID NO: 2679. In some embodiments, the AAV particle comprises a viral genome comprising SEQ ID NO: 2068 and a capsid comprising SEQ ID NO: 2809. In some embodiments, the AAV particle comprises a viral genome comprising SEQ ID NO: 2068 and a capsid comprising SEQ ID NO: 2871.

In some embodiments, the AAV particle comprises a viral genome comprising SEQ ID NO: 2069 and a VOY101 capsid. In some embodiments, the AAV particle comprises a viral genome comprising SEQ ID NO: 2069 and a VOY201 capsid. In some embodiments, the AAV particle comprises a viral genome comprising SEQ ID NO: 2069 and an AAV9 capsid. In some embodiments, the AAV particle comprises a viral genome comprising SEQ ID NO: 2069 and an AAV9K449R capsid. In some embodiments, the AAV particle comprises a viral genome comprising SEQ ID NO: 2069 and an AAVPHP.B capsid. In some embodiments, the AAV particle comprises a viral genome comprising SEQ ID NO: 2069 and an AAVPHP.N capsid. In some embodiments, the AAV particle comprises a viral genome comprising SEQ ID NO: 2069 and an AAV2 variant capsid. In some embodiments, the AAV particle comprises a viral genome comprising SEQ ID NO: 2069 and an AAV2/3 variant capsid. In some embodiments, the AAV particle comprises a viral genome comprising SEQ ID NO: 2069 and a capsid comprising SEQ ID NO: 1. In some embodiments, the AAV particle comprises a viral genome comprising SEQ ID NO: 2069 and a capsid encoded by a nucleic acid sequence comprising SEQ ID NO: 2. In some embodiments, the AAV particle comprises a viral genome comprising SEQ ID NO: 2069 and a capsid comprising SEQ ID NO: 4534. In some embodiments, the AAV particle comprises a viral genome comprising SEQ ID NO: 2069 and a capsid encoded by a nucleic acid sequence comprising SEQ ID NO: 3. In some embodiments, the AAV particle comprises a viral genome comprising SEQ ID NO: 2069 and a capsid comprising SEQ ID NO: 138. In some embodiments, the AAV particle comprises a viral genome comprising SEQ ID NO: 2069 and a capsid encoded by a nucleic acid sequence comprising SEQ ID NO: 137. In some embodiments, the AAV particle comprises a viral genome comprising SEQ ID NO: 2069 and a capsid comprising SEQ ID NO: 5. In some embodiments, the AAV particle comprises a viral genome comprising SEQ ID NO: 2069 and a capsid encoded by a nucleic acid sequence comprising SEQ ID NO: 6. In some embodiments, the AAV particle comprises a viral genome comprising SEQ ID NO: 2069 and a capsid comprising SEQ ID NO: 4. In some embodiments, the AAV particle comprises a viral genome comprising SEQ ID NO: 2069 and a capsid comprising SEQ ID NO: 11. In some embodiments, the AAV particle comprises a viral genome comprising SEQ ID NO: 2069 and a capsid comprising SEQ ID NO: 2679. In some embodiments, the AAV particle comprises a viral genome comprising SEQ ID NO: 2069 and a capsid comprising SEQ ID NO: 2809. In some embodiments, the AAV particle comprises a viral genome comprising SEQ ID NO: 2069 and a capsid comprising SEQ ID NO: 2871.

In some embodiments, the AAV particle comprises a viral genome comprising SEQ ID NO: 2070 and a VOY101 capsid. In some embodiments, the AAV particle comprises a viral genome comprising SEQ ID NO: 2070 and a VOY201 capsid. In some embodiments, the AAV particle comprises a viral genome comprising SEQ ID NO: 2070 and an AAV9 capsid. In some embodiments, the AAV particle comprises a viral genome comprising SEQ ID NO: 2070 and an AAV9K449R capsid. In some embodiments, the AAV particle comprises a viral genome comprising SEQ ID NO: 2070 and an AAVPHP.B capsid. In some embodiments, the AAV particle comprises a viral genome comprising SEQ ID NO: 2070 and an AAVPHP.N capsid. In some embodiments, the AAV particle comprises a viral genome comprising SEQ ID NO: 2070 and an AAV2 variant capsid. In some embodiments, the AAV particle comprises a viral genome comprising SEQ ID NO: 2070 and an AAV2/3 variant capsid. In some embodiments, the AAV particle comprises a viral genome comprising SEQ ID NO: 2070 and a capsid comprising SEQ ID NO: 1. In some embodiments, the AAV particle comprises a viral genome comprising SEQ ID NO: 2070 and a capsid encoded by a nucleic acid sequence comprising SEQ ID NO: 2. In some embodiments, the AAV particle comprises a viral genome comprising SEQ ID NO: 2070 and a capsid comprising SEQ ID NO: 4534. In some embodiments, the AAV particle comprises a viral genome comprising SEQ ID NO: 2070 and a capsid encoded by a nucleic acid sequence comprising SEQ ID NO: 3. In some embodiments, the AAV particle comprises a viral genome comprising SEQ ID NO: 2070 and a capsid comprising SEQ ID NO: 138. In some embodiments, the AAV particle comprises a viral genome comprising SEQ ID NO: 2070 and a capsid encoded by a nucleic acid sequence comprising SEQ ID NO: 137. In some embodiments, the AAV particle comprises a viral genome comprising SEQ ID NO: 2070 and a capsid comprising SEQ ID NO: 5. In some embodiments, the AAV particle comprises a viral genome comprising SEQ ID NO: 2070 and a capsid encoded by a nucleic acid sequence comprising SEQ ID NO: 6. In some embodiments, the AAV particle comprises a viral genome comprising SEQ ID NO: 2070 and a capsid comprising SEQ ID NO: 4. In some embodiments, the AAV particle comprises a viral genome comprising SEQ ID NO: 2070 and a capsid comprising SEQ ID NO: 11. In some embodiments, the AAV particle comprises a viral genome comprising SEQ ID NO: 2070 and a capsid comprising SEQ ID NO: 2679. In some embodiments, the AAV particle comprises a viral genome comprising SEQ ID NO: 2070 and a capsid comprising SEQ ID NO: 2809. In some embodiments, the AAV particle comprises a viral genome comprising SEQ ID NO: 2070 and a capsid comprising SEQ ID NO: 2871.

In some embodiments, the AAV particle comprises a viral genome comprising SEQ ID NO: 2071 and a VOY101 capsid. In some embodiments, the AAV particle comprises a viral genome comprising SEQ ID NO: 2071 and a VOY201 capsid. In some embodiments, the AAV particle comprises a viral genome comprising SEQ ID NO: 2071 and an AAV9 capsid. In some embodiments, the AAV particle comprises a viral genome comprising SEQ ID NO: 2071 and an AAV9K449R capsid. In some embodiments, the AAV particle comprises a viral genome comprising SEQ ID NO: 2071 and an AAVPHP.B capsid. In some embodiments, the AAV particle comprises a viral genome comprising SEQ ID NO: 2071 and an AAVPHP.N capsid. In some embodiments, the AAV particle comprises a viral genome comprising SEQ ID NO: 2071 and an AAV2 variant capsid. In some embodiments, the AAV particle comprises a viral genome comprising SEQ ID NO: 2071 and an AAV2/3 variant capsid. In some embodiments, the AAV particle comprises a viral genome comprising SEQ ID NO: 2071 and a capsid comprising SEQ ID NO: 1. In some embodiments, the AAV particle comprises a viral genome comprising SEQ ID NO: 2071 and a capsid encoded by a nucleic acid sequence comprising SEQ ID NO: 2. In some embodiments, the AAV particle comprises a viral genome comprising SEQ ID NO: 2071 and a capsid comprising SEQ ID NO: 4534. In some embodiments, the AAV particle comprises a viral genome comprising SEQ ID NO: 2071 and a capsid encoded by a nucleic acid sequence comprising SEQ ID NO: 3. In some embodiments, the AAV particle comprises a viral genome comprising SEQ ID NO: 2071 and a capsid comprising SEQ ID NO: 138. In some embodiments, the AAV particle comprises a viral genome comprising SEQ ID NO: 2071 and a capsid encoded by a nucleic acid sequence comprising SEQ ID NO: 137. In some embodiments, the AAV particle comprises a viral genome comprising SEQ ID NO: 2071 and a capsid comprising SEQ ID NO: 5. In some embodiments, the AAV particle comprises a viral genome comprising SEQ ID NO: 2071 and a capsid encoded by a nucleic acid sequence comprising SEQ ID NO: 6. In some embodiments, the AAV particle comprises a viral genome comprising SEQ ID NO: 2071 and a capsid comprising SEQ ID NO: 4. In some embodiments, the AAV particle comprises a viral genome comprising SEQ ID NO: 2071 and a capsid comprising SEQ ID NO: 11. In some embodiments, the AAV particle comprises a viral genome comprising SEQ ID NO: 2071 and a capsid comprising SEQ ID NO: 2679. In some embodiments, the AAV particle comprises a viral genome comprising SEQ ID NO: 2071 and a capsid comprising SEQ ID NO: 2809. In some embodiments, the AAV particle comprises a viral genome comprising SEQ ID NO: 2071 and a capsid comprising SEQ ID NO: 2871.

In some embodiments, the AAV particle comprises a viral genome comprising SEQ ID NO: 2072 and a VOY101 capsid. In some embodiments, the AAV particle comprises a viral genome comprising SEQ ID NO: 2072 and a VOY201 capsid. In some embodiments, the AAV particle comprises a viral genome comprising SEQ ID NO: 2072 and an AAV9 capsid. In some embodiments, the AAV particle comprises a viral genome comprising SEQ ID NO: 2072 and an AAV9K449R capsid. In some embodiments, the AAV particle comprises a viral genome comprising SEQ ID NO: 2072 and an AAVPHP.B capsid. In some embodiments, the AAV particle comprises a viral genome comprising SEQ ID NO: 2072 and an AAVPHP.N capsid. In some embodiments, the AAV particle comprises a viral genome comprising SEQ ID NO: 2072 and an AAV2 variant capsid. In some embodiments, the AAV particle comprises a viral genome comprising SEQ ID NO: 2072 and an AAV2/3 variant capsid. In some embodiments, the AAV particle comprises a viral genome comprising SEQ ID NO: 2072 and a capsid comprising SEQ ID NO: 1. In some embodiments, the AAV particle comprises a viral genome comprising SEQ ID NO: 2072 and a capsid encoded by a nucleic acid sequence comprising SEQ ID NO: 2. In some embodiments, the AAV particle comprises a viral genome comprising SEQ ID NO: 2072 and a capsid comprising SEQ ID NO: 4534. In some embodiments, the AAV particle comprises a viral genome comprising SEQ ID NO: 2072 and a capsid encoded by a nucleic acid sequence comprising SEQ ID NO: 3. In some embodiments, the AAV particle comprises a viral genome comprising SEQ ID NO: 2072 and a capsid comprising SEQ ID NO: 138. In some embodiments, the AAV particle comprises a viral genome comprising SEQ ID NO: 2072 and a capsid encoded by a nucleic acid sequence comprising SEQ ID NO: 137. In some embodiments, the AAV particle comprises a viral genome comprising SEQ ID NO: 2072 and a capsid comprising SEQ ID NO: 5. In some embodiments, the AAV particle comprises a viral genome comprising SEQ ID NO: 2072 and a capsid encoded by a nucleic acid sequence comprising SEQ ID NO: 6. In some embodiments, the AAV particle comprises a viral genome comprising SEQ ID NO: 2072 and a capsid comprising SEQ ID NO: 4. In some embodiments, the AAV particle comprises a viral genome comprising SEQ ID NO: 2072 and a capsid comprising SEQ ID NO: 11. In some embodiments, the AAV particle comprises a viral genome comprising SEQ ID NO: 2072 and a capsid comprising SEQ ID NO: 2679. In some embodiments, the AAV particle comprises a viral genome comprising SEQ ID NO: 2072 and a capsid comprising SEQ ID NO: 2809. In some embodiments, the AAV particle comprises a viral genome comprising SEQ ID NO: 2072 and a capsid comprising SEQ ID NO: 2871.

In some embodiments, the AAV particle comprises a viral genome comprising SEQ ID NO: 2073 and a VOY101 capsid. In some embodiments, the AAV particle comprises a viral genome comprising SEQ ID NO: 2073 and a VOY201 capsid. In some embodiments, the AAV particle comprises a viral genome comprising SEQ ID NO: 2073 and an AAV9 capsid. In some embodiments, the AAV particle comprises a viral genome comprising SEQ ID NO: 2073 and an AAV9K449R capsid. In some embodiments, the AAV particle comprises a viral genome comprising SEQ ID NO: 2073 and an AAVPHP.B capsid. In some embodiments, the AAV particle comprises a viral genome comprising SEQ ID NO: 2073 and an AAVPHP.N capsid. In some embodiments, the AAV particle comprises a viral genome comprising SEQ ID NO: 2073 and an AAV2 variant capsid. In some embodiments, the AAV particle comprises a viral genome comprising SEQ ID NO: 2073 and an AAV2/3 variant capsid. In some embodiments, the AAV particle comprises a viral genome comprising SEQ ID NO: 2073 and a capsid comprising SEQ ID NO: 1. In some embodiments, the AAV particle comprises a viral genome comprising SEQ ID NO: 2073 and a capsid encoded by a nucleic acid sequence comprising SEQ ID NO: 2. In some embodiments, the AAV particle comprises a viral genome comprising SEQ ID NO: 2073 and a capsid comprising SEQ ID NO: 4534. In some embodiments, the AAV particle comprises a viral genome comprising SEQ ID NO: 2073 and a capsid encoded by a nucleic acid sequence comprising SEQ ID NO: 3. In some embodiments, the AAV particle comprises a viral genome comprising SEQ ID NO: 2073 and a capsid comprising SEQ ID NO: 138. In some embodiments, the AAV particle comprises a viral genome comprising SEQ ID NO: 2073 and a capsid encoded by a nucleic acid sequence comprising SEQ ID NO: 137. In some embodiments, the AAV particle comprises a viral genome comprising SEQ ID NO: 2073 and a capsid comprising SEQ ID NO: 5. In some embodiments, the AAV particle comprises a viral genome comprising SEQ ID NO: 2073 and a capsid encoded by a nucleic acid sequence comprising SEQ ID NO: 6. In some embodiments, the AAV particle comprises a viral genome comprising SEQ ID NO: 2073 and a capsid comprising SEQ ID NO: 4. In some embodiments, the AAV particle comprises a viral genome comprising SEQ ID NO: 2073 and a capsid comprising SEQ ID NO: 11. In some embodiments, the AAV particle comprises a viral genome comprising SEQ ID NO: 2073 and a capsid comprising SEQ ID NO: 2679. In some embodiments, the AAV particle comprises a viral genome comprising SEQ ID NO: 2073 and a capsid comprising SEQ ID NO: 2809. In some embodiments, the AAV particle comprises a viral genome comprising SEQ ID NO: 2073 and a capsid comprising SEQ ID NO: 2871.

In some embodiments, the AAV particle comprises a viral genome comprising SEQ ID NO: 2074 and a VOY101 capsid. In some embodiments, the AAV particle comprises a viral genome comprising SEQ ID NO: 2074 and a VOY201 capsid. In some embodiments, the AAV particle comprises a viral genome comprising SEQ ID NO: 2074 and an AAV9 capsid. In some embodiments, the AAV particle comprises a viral genome comprising SEQ ID NO: 2074 and an AAV9K449R capsid. In some embodiments, the AAV particle comprises a viral genome comprising SEQ ID NO: 2074 and an AAVPHP.B capsid. In some embodiments, the AAV particle comprises a viral genome comprising SEQ ID NO: 2074 and an AAVPHP.N capsid. In some embodiments, the AAV particle comprises a viral genome comprising SEQ ID NO: 2074 and an AAV2 variant capsid. In some embodiments, the AAV particle comprises a viral genome comprising SEQ ID NO: 2074 and an AAV2/3 variant capsid. In some embodiments, the AAV particle comprises a viral genome comprising SEQ ID NO: 2074 and a capsid comprising SEQ ID NO: 1. In some embodiments, the AAV particle comprises a viral genome comprising SEQ ID NO: 2074 and a capsid encoded by a nucleic acid sequence comprising SEQ ID NO: 2. In some embodiments, the AAV particle comprises a viral genome comprising SEQ ID NO: 2074 and a capsid comprising SEQ ID NO: 4534. In some embodiments, the AAV particle comprises a viral genome comprising SEQ ID NO: 2074 and a capsid encoded by a nucleic acid sequence comprising SEQ ID NO: 3. In some embodiments, the AAV particle comprises a viral genome comprising SEQ ID NO: 2074 and a capsid comprising SEQ ID NO: 138. In some embodiments, the AAV particle comprises a viral genome comprising SEQ ID NO: 2074 and a capsid encoded by a nucleic acid sequence comprising SEQ ID NO: 137. In some embodiments, the AAV particle comprises a viral genome comprising SEQ ID NO: 2074 and a capsid comprising SEQ ID NO: 5. In some embodiments, the AAV particle comprises a viral genome comprising SEQ ID NO: 2074 and a capsid encoded by a nucleic acid sequence comprising SEQ ID NO: 6. In some embodiments, the AAV particle comprises a viral genome comprising SEQ ID NO: 2074 and a capsid comprising SEQ ID NO: 4. In some embodiments, the AAV particle comprises a viral genome comprising SEQ ID NO: 2074 and a capsid comprising SEQ ID NO: 11. In some embodiments, the AAV particle comprises a viral genome comprising SEQ ID NO: 2074 and a capsid comprising SEQ ID NO: 2679. In some embodiments, the AAV particle comprises a viral genome comprising SEQ ID NO: 2074 and a capsid comprising SEQ ID NO: 2809. In some embodiments, the AAV particle comprises a viral genome comprising SEQ ID NO: 2074 and a capsid comprising SEQ ID NO: 2871.

In some embodiments, the AAV particle comprises a viral genome comprising SEQ ID NO: 2075 and a VOY101 capsid. In some embodiments, the AAV particle comprises a viral genome comprising SEQ ID NO: 2075 and a VOY201 capsid. In some embodiments, the AAV particle comprises a viral genome comprising SEQ ID NO: 2075 and an AAV9 capsid. In some embodiments, the AAV particle comprises a viral genome comprising SEQ ID NO: 2075 and an AAV9K449R capsid. In some embodiments, the AAV particle comprises a viral genome comprising SEQ ID NO: 2075 and an AAVPHP.B capsid. In some embodiments, the AAV particle comprises a viral genome comprising SEQ ID NO: 2075 and an AAVPHP.N capsid. In some embodiments, the AAV particle comprises a viral genome comprising SEQ ID NO: 2075 and an AAV2 variant capsid. In some embodiments, the AAV particle comprises a viral genome comprising SEQ ID NO: 2075 and an AAV2/3 variant capsid. In some embodiments, the AAV particle comprises a viral genome comprising SEQ ID NO: 2075 and a capsid comprising SEQ ID NO: 1. In some embodiments, the AAV particle comprises a viral genome comprising SEQ ID NO: 2075 and a capsid encoded by a nucleic acid sequence comprising SEQ ID NO: 2. In some embodiments, the AAV particle comprises a viral genome comprising SEQ ID NO: 2075 and a capsid comprising SEQ ID NO: 4534. In some embodiments, the AAV particle comprises a viral genome comprising SEQ ID NO: 2075 and a capsid encoded by a nucleic acid sequence comprising SEQ ID NO: 3. In some embodiments, the AAV particle comprises a viral genome comprising SEQ ID NO: 2075 and a capsid comprising SEQ ID NO: 138. In some embodiments, the AAV particle comprises a viral genome comprising SEQ ID NO: 2075 and a capsid encoded by a nucleic acid sequence comprising SEQ ID NO: 137. In some embodiments, the AAV particle comprises a viral genome comprising SEQ ID NO: 2075 and a capsid comprising SEQ ID NO: 5. In some embodiments, the AAV particle comprises a viral genome comprising SEQ ID NO: 2075 and a capsid encoded by a nucleic acid sequence comprising SEQ ID NO: 6. In some embodiments, the AAV particle comprises a viral genome comprising SEQ ID NO: 2075 and a capsid comprising SEQ ID NO: 4. In some embodiments, the AAV particle comprises a viral genome comprising SEQ ID NO: 2075 and a capsid comprising SEQ ID NO: 11. In some embodiments, the AAV particle comprises a viral genome comprising SEQ ID NO: 2075 and a capsid comprising SEQ ID NO: 2679. In some embodiments, the AAV particle comprises a viral genome comprising SEQ ID NO: 2075 and a capsid comprising SEQ ID NO: 2809. In some embodiments, the AAV particle comprises a viral genome comprising SEQ ID NO: 2075 and a capsid comprising SEQ ID NO: 2871.

In some embodiments, the AAV particle comprises a viral genome comprising SEQ ID NO: 2137 and a VOY101 capsid. In some embodiments, the AAV particle comprises a viral genome comprising SEQ ID NO: 2137 and a VOY201 capsid. In some embodiments, the AAV particle comprises a viral genome comprising SEQ ID NO: 2137 and an AAV9 capsid. In some embodiments, the AAV particle comprises a viral genome comprising SEQ ID NO: 2137 and an AAV9K449R capsid. In some embodiments, the AAV particle comprises a viral genome comprising SEQ ID NO: 2137 and an AAVPHP.B capsid. In some embodiments, the AAV particle comprises a viral genome comprising SEQ ID NO: 2137 and an AAVPHP.N capsid. In some embodiments, the AAV particle comprises a viral genome comprising SEQ ID NO: 2137 and an AAV2 variant capsid. In some embodiments, the AAV particle comprises a viral genome comprising SEQ ID NO: 2137 and an AAV2/3 variant capsid. In some embodiments, the AAV particle comprises a viral genome comprising SEQ ID NO: 2137 and a capsid comprising SEQ ID NO: 1. In some embodiments, the AAV particle comprises a viral genome comprising SEQ ID NO: 2137 and a capsid encoded by a nucleic acid sequence comprising SEQ ID NO: 2. In some embodiments, the AAV particle comprises a viral genome comprising SEQ ID NO: 2137 and a capsid comprising SEQ ID NO: 4534. In some embodiments, the AAV particle comprises a viral genome comprising SEQ ID NO: 2137 and a capsid encoded by a nucleic acid sequence comprising SEQ ID NO: 3. In some embodiments, the AAV particle comprises a viral genome comprising SEQ ID NO: 2137 and a capsid comprising SEQ ID NO: 138. In some embodiments, the AAV particle comprises a viral genome comprising SEQ ID NO: 2137 and a capsid encoded by a nucleic acid sequence comprising SEQ ID NO: 137. In some embodiments, the AAV particle comprises a viral genome comprising SEQ ID NO: 2137 and a capsid comprising SEQ ID NO: 5. In some embodiments, the AAV particle comprises a viral genome comprising SEQ ID NO: 2137 and a capsid encoded by a nucleic acid sequence comprising SEQ ID NO: 6. In some embodiments, the AAV particle comprises a viral genome comprising SEQ ID NO: 2137 and a capsid comprising SEQ ID NO: 4. In some embodiments, the AAV particle comprises a viral genome comprising SEQ ID NO: 2137 and a capsid comprising SEQ ID NO: 11. In some embodiments, the AAV particle comprises a viral genome comprising SEQ ID NO: 2137 and a capsid comprising SEQ ID NO: 2679. In some embodiments, the AAV particle comprises a viral genome comprising SEQ ID NO: 2137 and a capsid comprising SEQ ID NO: 2809. In some embodiments, the AAV particle comprises a viral genome comprising SEQ ID NO: 2137 and a capsid comprising SEQ ID NO: 2871.

In some embodiments, the AAV particle comprises a viral genome comprising SEQ ID NO: 2138 and a VOY101 capsid. In some embodiments, the AAV particle comprises a viral genome comprising SEQ ID NO: 2138 and a VOY201 capsid. In some embodiments, the AAV particle comprises a viral genome comprising SEQ ID NO: 2138 and an AAV9 capsid. In some embodiments, the AAV particle comprises a viral genome comprising SEQ ID NO: 2138 and an AAV9K449R capsid. In some embodiments, the AAV particle comprises a viral genome comprising SEQ ID NO: 2138 and an AAVPHP.B capsid. In some embodiments, the AAV particle comprises a viral genome comprising SEQ ID NO: 2138 and an AAVPHP.N capsid. In some embodiments, the AAV particle comprises a viral genome comprising SEQ ID NO: 2138 and an AAV2 variant capsid. In some embodiments, the AAV particle comprises a viral genome comprising SEQ ID NO: 2138 and an AAV2/3 variant capsid. In some embodiments, the AAV particle comprises a viral genome comprising SEQ ID NO: 2138 and a capsid comprising SEQ ID NO: 1. In some embodiments, the AAV particle comprises a viral genome comprising SEQ ID NO: 2138 and a capsid encoded by a nucleic acid sequence comprising SEQ ID NO: 2. In some embodiments, the AAV particle comprises a viral genome comprising SEQ ID NO: 2138 and a capsid comprising SEQ ID NO: 4534. In some embodiments, the AAV particle comprises a viral genome comprising SEQ ID NO: 2138 and a capsid encoded by a nucleic acid sequence comprising SEQ ID NO: 3. In some embodiments, the AAV particle comprises a viral genome comprising SEQ ID NO: 2138 and a capsid comprising SEQ ID NO: 138. In some embodiments, the AAV particle comprises a viral genome comprising SEQ ID NO: 2138 and a capsid encoded by a nucleic acid sequence comprising SEQ ID NO: 137. In some embodiments, the AAV particle comprises a viral genome comprising SEQ ID NO: 2138 and a capsid comprising SEQ ID NO: 5. In some embodiments, the AAV particle comprises a viral genome comprising SEQ ID NO: 2138 and a capsid encoded by a nucleic acid sequence comprising SEQ ID NO: 6. In some embodiments, the AAV particle comprises a viral genome comprising SEQ ID NO: 2138 and a capsid comprising SEQ ID NO: 4. In some embodiments, the AAV particle comprises a viral genome comprising SEQ ID NO: 2138 and a capsid comprising SEQ ID NO: 11. In some embodiments, the AAV particle comprises a viral genome comprising SEQ ID NO: 2138 and a capsid comprising SEQ ID NO: 2679. In some embodiments, the AAV particle comprises a viral genome comprising SEQ ID NO: 2138 and a capsid comprising SEQ ID NO: 2809. In some embodiments, the AAV particle comprises a viral genome comprising SEQ ID NO: 2138 and a capsid comprising SEQ ID NO: 2871.

In some embodiments, the AAV particle comprises a viral genome comprising SEQ ID NO: 2139 and a VOY101 capsid. In some embodiments, the AAV particle comprises a viral genome comprising SEQ ID NO: 2139 and a VOY201 capsid. In some embodiments, the AAV particle comprises a viral genome comprising SEQ ID NO: 2139 and an AAV9 capsid. In some embodiments, the AAV particle comprises a viral genome comprising SEQ ID NO: 2139 and an AAV9K449R capsid. In some embodiments, the AAV particle comprises a viral genome comprising SEQ ID NO: 2139 and an AAVPHP.B capsid. In some embodiments, the AAV particle comprises a viral genome comprising SEQ ID NO: 2139 and an AAVPHP.N capsid. In some embodiments, the AAV particle comprises a viral genome comprising SEQ ID NO: 2139 and an AAV2 variant capsid. In some embodiments, the AAV particle comprises a viral genome comprising SEQ ID NO: 2139 and an AAV2/3 variant capsid. In some embodiments, the AAV particle comprises a viral genome comprising SEQ ID NO: 2139 and a capsid comprising SEQ ID NO: 1. In some embodiments, the AAV particle comprises a viral genome comprising SEQ ID NO: 2139 and a capsid encoded by a nucleic acid sequence comprising SEQ ID NO: 2. In some embodiments, the AAV particle comprises a viral genome comprising SEQ ID NO: 2139 and a capsid comprising SEQ ID NO: 4534. In some embodiments, the AAV particle comprises a viral genome comprising SEQ ID NO: 2139 and a capsid encoded by a nucleic acid sequence comprising SEQ ID NO: 3. In some embodiments, the AAV particle comprises a viral genome comprising SEQ ID NO: 2139 and a capsid comprising SEQ ID NO: 138. In some embodiments, the AAV particle comprises a viral genome comprising SEQ ID NO: 2139 and a capsid encoded by a nucleic acid sequence comprising SEQ ID NO: 137. In some embodiments, the AAV particle comprises a viral genome comprising SEQ ID NO: 2139 and a capsid comprising SEQ ID NO: 5. In some embodiments, the AAV particle comprises a viral genome comprising SEQ ID NO: 2139 and a capsid encoded by a nucleic acid sequence comprising SEQ ID NO: 6. In some embodiments, the AAV particle comprises a viral genome comprising SEQ ID NO: 2139 and a capsid comprising SEQ ID NO: 4. In some embodiments, the AAV particle comprises a viral genome comprising SEQ ID NO: 2139 and a capsid comprising SEQ ID NO: 11. In some embodiments, the AAV particle comprises a viral genome comprising SEQ ID NO: 2139 and a capsid comprising SEQ ID NO: 2679. In some embodiments, the AAV particle comprises a viral genome comprising SEQ ID NO: 2139 and a capsid comprising SEQ ID NO: 2809. In some embodiments, the AAV particle comprises a viral genome comprising SEQ ID NO: 2139 and a capsid comprising SEQ ID NO: 2871.

In some embodiments, the AAV particle comprises a viral genome comprising SEQ ID NO: 2140 and a VOY101 capsid. In some embodiments, the AAV particle comprises a viral genome comprising SEQ ID NO: 2140 and a VOY201 capsid. In some embodiments, the AAV particle comprises a viral genome comprising SEQ ID NO: 2140 and an AAV9 capsid. In some embodiments, the AAV particle comprises a viral genome comprising SEQ ID NO: 2140 and an AAV9K449R capsid. In some embodiments, the AAV particle comprises a viral genome comprising SEQ ID NO: 2140 and an AAVPHP.B capsid. In some embodiments, the AAV particle comprises a viral genome comprising SEQ ID NO: 2140 and an AAVPHP.N capsid. In some embodiments, the AAV particle comprises a viral genome comprising SEQ ID NO: 2140 and an AAV2 variant capsid. In some embodiments, the AAV particle comprises a viral genome comprising SEQ ID NO: 2140 and an AAV2/3 variant capsid. In some embodiments, the AAV particle comprises a viral genome comprising SEQ ID NO: 2140 and a capsid comprising SEQ ID NO: 1. In some embodiments, the AAV particle comprises a viral genome comprising SEQ ID NO: 2140 and a capsid encoded by a nucleic acid sequence comprising SEQ ID NO: 2. In some embodiments, the AAV particle comprises a viral genome comprising SEQ ID NO: 2140 and a capsid comprising SEQ ID NO: 4534. In some embodiments, the AAV particle comprises a viral genome comprising SEQ ID NO: 2140 and a capsid encoded by a nucleic acid sequence comprising SEQ ID NO: 3. In some embodiments, the AAV particle comprises a viral genome comprising SEQ ID NO: 2140 and a capsid comprising SEQ ID NO: 138. In some embodiments, the AAV particle comprises a viral genome comprising SEQ ID NO: 2140 and a capsid encoded by a nucleic acid sequence comprising SEQ ID NO: 137. In some embodiments, the AAV particle comprises a viral genome comprising SEQ ID NO: 2140 and a capsid comprising SEQ ID NO: 5. In some embodiments, the AAV particle comprises a viral genome comprising SEQ ID NO: 2140 and a capsid encoded by a nucleic acid sequence comprising SEQ ID NO: 6. In some embodiments, the AAV particle comprises a viral genome comprising SEQ ID NO: 2140 and a capsid comprising SEQ ID NO: 4. In some embodiments, the AAV particle comprises a viral genome comprising SEQ ID NO: 2140 and a capsid comprising SEQ ID NO: 11. In some embodiments, the AAV particle comprises a viral genome comprising SEQ ID NO: 2140 and a capsid comprising SEQ ID NO: 2679. In some embodiments, the AAV particle comprises a viral genome comprising SEQ ID NO: 2140 and a capsid comprising SEQ ID NO: 2809. In some embodiments, the AAV particle comprises a viral genome comprising SEQ ID NO: 2140 and a capsid comprising SEQ ID NO: 2871.

In some embodiments, the AAV particle comprises a viral genome comprising SEQ ID NO: 2141 and a VOY101 capsid. In some embodiments, the AAV particle comprises a viral genome comprising SEQ ID NO: 2141 and a VOY201 capsid. In some embodiments, the AAV particle comprises a viral genome comprising SEQ ID NO: 2141 and an AAV9 capsid. In some embodiments, the AAV particle comprises a viral genome comprising SEQ ID NO: 2141 and an AAV9K449R capsid. In some embodiments, the AAV particle comprises a viral genome comprising SEQ ID NO: 2141 and an AAVPHP.B capsid. In some embodiments, the AAV particle comprises a viral genome comprising SEQ ID NO: 2141 and an AAVPHP.N capsid. In some embodiments, the AAV particle comprises a viral genome comprising SEQ ID NO: 2141 and an AAV2 variant capsid. In some embodiments, the AAV particle comprises a viral genome comprising SEQ ID NO: 2141 and an AAV2/3 variant capsid. In some embodiments, the AAV particle comprises a viral genome comprising SEQ ID NO: 2141 and a capsid comprising SEQ ID NO: 1. In some embodiments, the AAV particle comprises a viral genome comprising SEQ ID NO: 2141 and a capsid encoded by a nucleic acid sequence comprising SEQ ID NO: 2. In some embodiments, the AAV particle comprises a viral genome comprising SEQ ID NO: 2141 and a capsid comprising SEQ ID NO: 4534. In some embodiments, the AAV particle comprises a viral genome comprising SEQ ID NO: 2141 and a capsid encoded by a nucleic acid sequence comprising SEQ ID NO: 3. In some embodiments, the AAV particle comprises a viral genome comprising SEQ ID NO: 2141 and a capsid comprising SEQ ID NO: 138. In some embodiments, the AAV particle comprises a viral genome comprising SEQ ID NO: 2141 and a capsid encoded by a nucleic acid sequence comprising SEQ ID NO: 137. In some embodiments, the AAV particle comprises a viral genome comprising SEQ ID NO: 2141 and a capsid comprising SEQ ID NO: 5. In some embodiments, the AAV particle comprises a viral genome comprising SEQ ID NO: 2141 and a capsid encoded by a nucleic acid sequence comprising SEQ ID NO: 6. In some embodiments, the AAV particle comprises a viral genome comprising SEQ ID NO: 2141 and a capsid comprising SEQ ID NO: 4. In some embodiments, the AAV particle comprises a viral genome comprising SEQ ID NO: 2141 and a capsid comprising SEQ ID NO: 11. In some embodiments, the AAV particle comprises a viral genome comprising SEQ ID NO: 2141 and a capsid comprising SEQ ID NO: 2679. In some embodiments, the AAV particle comprises a viral genome comprising SEQ ID NO: 2141 and a capsid comprising SEQ ID NO: 2809. In some embodiments, the AAV particle comprises a viral genome comprising SEQ ID NO: 2141 and a capsid comprising SEQ ID NO: 2871.

In some embodiments, the AAV particle comprises a viral genome comprising SEQ ID NO: 2142 and a VOY101 capsid. In some embodiments, the AAV particle comprises a viral genome comprising SEQ ID NO: 2142 and a VOY201 capsid. In some embodiments, the AAV particle comprises a viral genome comprising SEQ ID NO: 2142 and an AAV9 capsid. In some embodiments, the AAV particle comprises a viral genome comprising SEQ ID NO: 2142 and an AAV9K449R capsid. In some embodiments, the AAV particle comprises a viral genome comprising SEQ ID NO: 2142 and an AAVPHP.B capsid. In some embodiments, the AAV particle comprises a viral genome comprising SEQ ID NO: 2142 and an AAVPHP.N capsid. In some embodiments, the AAV particle comprises a viral genome comprising SEQ ID NO: 2142 and an AAV2 variant capsid. In some embodiments, the AAV particle comprises a viral genome comprising SEQ ID NO: 2142 and an AAV2/3 variant capsid. In some embodiments, the AAV particle comprises a viral genome comprising SEQ ID NO: 2142 and a capsid comprising SEQ ID NO: 1. In some embodiments, the AAV particle comprises a viral genome comprising SEQ ID NO: 2142 and a capsid encoded by a nucleic acid sequence comprising SEQ ID NO: 2. In some embodiments, the AAV particle comprises a viral genome comprising SEQ ID NO: 2142 and a capsid comprising SEQ ID NO: 4534. In some embodiments, the AAV particle comprises a viral genome comprising SEQ ID NO: 2142 and a capsid encoded by a nucleic acid sequence comprising SEQ ID NO: 3. In some embodiments, the AAV particle comprises a viral genome comprising SEQ ID NO: 2142 and a capsid comprising SEQ ID NO: 138. In some embodiments, the AAV particle comprises a viral genome comprising SEQ ID NO: 2142 and a capsid encoded by a nucleic acid sequence comprising SEQ ID NO: 137. In some embodiments, the AAV particle comprises a viral genome comprising SEQ ID NO: 2142 and a capsid comprising SEQ ID NO: 5. In some embodiments, the AAV particle comprises a viral genome comprising SEQ ID NO: 2142 and a capsid encoded by a nucleic acid sequence comprising SEQ ID NO: 6. In some embodiments, the AAV particle comprises a viral genome comprising SEQ ID NO: 2142 and a capsid comprising SEQ ID NO: 4. In some embodiments, the AAV particle comprises a viral genome comprising SEQ ID NO: 2142 and a capsid comprising SEQ ID NO: 11. In some embodiments, the AAV particle comprises a viral genome comprising SEQ ID NO: 2142 and a capsid comprising SEQ ID NO: 2679. In some embodiments, the AAV particle comprises a viral genome comprising SEQ ID NO: 2142 and a capsid comprising SEQ ID NO: 2809. In some embodiments, the AAV particle comprises a viral genome comprising SEQ ID NO: 2142 and a capsid comprising SEQ ID NO: 2871.

In some embodiments, the AAV particle comprises a viral genome comprising SEQ ID NO: 2143 and a VOY101 capsid. In some embodiments, the AAV particle comprises a viral genome comprising SEQ ID NO: 2143 and a VOY201 capsid. In some embodiments, the AAV particle comprises a viral genome comprising SEQ ID NO: 2143 and an AAV9 capsid. In some embodiments, the AAV particle comprises a viral genome comprising SEQ ID NO: 2143 and an AAV9K449R capsid. In some embodiments, the AAV particle comprises a viral genome comprising SEQ ID NO: 2143 and an AAVPHP.B capsid. In some embodiments, the AAV particle comprises a viral genome comprising SEQ ID NO: 2143 and an AAVPHP.N capsid. In some embodiments, the AAV particle comprises a viral genome comprising SEQ ID NO: 2143 and an AAV2 variant capsid. In some embodiments, the AAV particle comprises a viral genome comprising SEQ ID NO: 2143 and an AAV2/3 variant capsid. In some embodiments, the AAV particle comprises a viral genome comprising SEQ ID NO: 2143 and a capsid comprising SEQ ID NO: 1. In some embodiments, the AAV particle comprises a viral genome comprising SEQ ID NO: 2143 and a capsid encoded by a nucleic acid sequence comprising SEQ ID NO: 2. In some embodiments, the AAV particle comprises a viral genome comprising SEQ ID NO: 2143 and a capsid comprising SEQ ID NO: 4534. In some embodiments, the AAV particle comprises a viral genome comprising SEQ ID NO: 2143 and a capsid encoded by a nucleic acid sequence comprising SEQ ID NO: 3. In some embodiments, the AAV particle comprises a viral genome comprising SEQ ID NO: 2143 and a capsid comprising SEQ ID NO: 138. In some embodiments, the AAV particle comprises a viral genome comprising SEQ ID NO: 2143 and a capsid encoded by a nucleic acid sequence comprising SEQ ID NO: 137. In some embodiments, the AAV particle comprises a viral genome comprising SEQ ID NO: 2143 and a capsid comprising SEQ ID NO: 5. In some embodiments, the AAV particle comprises a viral genome comprising SEQ ID NO: 2143 and a capsid encoded by a nucleic acid sequence comprising SEQ ID NO: 6. In some embodiments, the AAV particle comprises a viral genome comprising SEQ ID NO: 2143 and a capsid comprising SEQ ID NO: 4. In some embodiments, the AAV particle comprises a viral genome comprising SEQ ID NO: 2143 and a capsid comprising SEQ ID NO: 11. In some embodiments, the AAV particle comprises a viral genome comprising SEQ ID NO: 2143 and a capsid comprising SEQ ID NO: 2679. In some embodiments, the AAV particle comprises a viral genome comprising SEQ ID NO: 2143 and a capsid comprising SEQ ID NO: 2809. In some embodiments, the AAV particle comprises a viral genome comprising SEQ ID NO: 2143 and a capsid comprising SEQ ID NO: 2871.

In some embodiments, the AAV particle comprises a viral genome comprising SEQ ID NO: 2144 and a VOY101 capsid. In some embodiments, the AAV particle comprises a viral genome comprising SEQ ID NO: 2144 and a VOY201 capsid. In some embodiments, the AAV particle comprises a viral genome comprising SEQ ID NO: 2144 and an AAV9 capsid. In some embodiments, the AAV particle comprises a viral genome comprising SEQ ID NO: 2144 and an AAV9K449R capsid. In some embodiments, the AAV particle comprises a viral genome comprising SEQ ID NO: 2144 and an AAVPHP.B capsid. In some embodiments, the AAV particle comprises a viral genome comprising SEQ ID NO: 2144 and an AAVPHP.N capsid. In some embodiments, the AAV particle comprises a viral genome comprising SEQ ID NO: 2144 and an AAV2 variant capsid. In some embodiments, the AAV particle comprises a viral genome comprising SEQ ID NO: 2144 and an AAV2/3 variant capsid. In some embodiments, the AAV particle comprises a viral genome comprising SEQ ID NO: 2144 and a capsid comprising SEQ ID NO: 1. In some embodiments, the AAV particle comprises a viral genome comprising SEQ ID NO: 2144 and a capsid encoded by a nucleic acid sequence comprising SEQ ID NO: 2. In some embodiments, the AAV particle comprises a viral genome comprising SEQ ID NO: 2144 and a capsid comprising SEQ ID NO: 4534. In some embodiments, the AAV particle comprises a viral genome comprising SEQ ID NO: 2144 and a capsid encoded by a nucleic acid sequence comprising SEQ ID NO: 3. In some embodiments, the AAV particle comprises a viral genome comprising SEQ ID NO: 2144 and a capsid comprising SEQ ID NO: 138. In some embodiments, the AAV particle comprises a viral genome comprising SEQ ID NO: 2144 and a capsid encoded by a nucleic acid sequence comprising SEQ ID NO: 137. In some embodiments, the AAV particle comprises a viral genome comprising SEQ ID NO: 2144 and a capsid comprising SEQ ID NO: 5. In some embodiments, the AAV particle comprises a viral genome comprising SEQ ID NO: 2144 and a capsid encoded by a nucleic acid sequence comprising SEQ ID NO: 6. In some embodiments, the AAV particle comprises a viral genome comprising SEQ ID NO: 2144 and a capsid comprising SEQ ID NO: 4. In some embodiments, the AAV particle comprises a viral genome comprising SEQ ID NO: 2144 and a capsid comprising SEQ ID NO: 11. In some embodiments, the AAV particle comprises a viral genome comprising SEQ ID NO: 2144 and a capsid comprising SEQ ID NO: 2679. In some embodiments, the AAV particle comprises a viral genome comprising SEQ ID NO: 2144 and a capsid comprising SEQ ID NO: 2809. In some embodiments, the AAV particle comprises a viral genome comprising SEQ ID NO: 2144 and a capsid comprising SEQ ID NO: 2871.

In some embodiments, the AAV particle comprises a viral genome comprising SEQ ID NO: 2145 and a VOY101 capsid. In some embodiments, the AAV particle comprises a viral genome comprising SEQ ID NO: 2145 and a VOY201 capsid. In some embodiments, the AAV particle comprises a viral genome comprising SEQ ID NO: 2145 and an AAV9 capsid. In some embodiments, the AAV particle comprises a viral genome comprising SEQ ID NO: 2145 and an AAV9K449R capsid. In some embodiments, the AAV particle comprises a viral genome comprising SEQ ID NO: 2145 and an AAVPHP.B capsid. In some embodiments, the AAV particle comprises a viral genome comprising SEQ ID NO: 2145 and an AAVPHP.N capsid. In some embodiments, the AAV particle comprises a viral genome comprising SEQ ID NO: 2145 and an AAV2 variant capsid. In some embodiments, the AAV particle comprises a viral genome comprising SEQ ID NO: 2145 and an AAV2/3 variant capsid. In some embodiments, the AAV particle comprises a viral genome comprising SEQ ID NO: 2145 and a capsid comprising SEQ ID NO: 1. In some embodiments, the AAV particle comprises a viral genome comprising SEQ ID NO: 2145 and a capsid encoded by a nucleic acid sequence comprising SEQ ID NO: 2. In some embodiments, the AAV particle comprises a viral genome comprising SEQ ID NO: 2145 and a capsid comprising SEQ ID NO: 4534. In some embodiments, the AAV particle comprises a viral genome comprising SEQ ID NO: 2145 and a capsid encoded by a nucleic acid sequence comprising SEQ ID NO: 3. In some embodiments, the AAV particle comprises a viral genome comprising SEQ ID NO: 2145 and a capsid comprising SEQ ID NO: 138. In some embodiments, the AAV particle comprises a viral genome comprising SEQ ID NO: 2145 and a capsid encoded by a nucleic acid sequence comprising SEQ ID NO: 137. In some embodiments, the AAV particle comprises a viral genome comprising SEQ ID NO: 2145 and a capsid comprising SEQ ID NO: 5. In some embodiments, the AAV particle comprises a viral genome comprising SEQ ID NO: 2145 and a capsid encoded by a nucleic acid sequence comprising SEQ ID NO: 6. In some embodiments, the AAV particle comprises a viral genome comprising SEQ ID NO: 2145 and a capsid comprising SEQ ID NO: 4. In some embodiments, the AAV particle comprises a viral genome comprising SEQ ID NO: 2145 and a capsid comprising SEQ ID NO: 11. In some embodiments, the AAV particle comprises a viral genome comprising SEQ ID NO: 2145 and a capsid comprising SEQ ID NO: 2679. In some embodiments, the AAV particle comprises a viral genome comprising SEQ ID NO: 2145 and a capsid comprising SEQ ID NO: 2809. In some embodiments, the AAV particle comprises a viral genome comprising SEQ ID NO: 2145 and a capsid comprising SEQ ID NO: 2871.

In some embodiments, the AAV particle comprises a viral genome comprising SEQ ID NO: 2146 and a VOY101 capsid. In some embodiments, the AAV particle comprises a viral genome comprising SEQ ID NO: 2146 and a VOY201 capsid. In some embodiments, the AAV particle comprises a viral genome comprising SEQ ID NO: 2146 and an AAV9 capsid. In some embodiments, the AAV particle comprises a viral genome comprising SEQ ID NO: 2146 and an AAV9K449R capsid. In some embodiments, the AAV particle comprises a viral genome comprising SEQ ID NO: 2146 and an AAVPHP.B capsid. In some embodiments, the AAV particle comprises a viral genome comprising SEQ ID NO: 2146 and an AAVPHP.N capsid. In some embodiments, the AAV particle comprises a viral genome comprising SEQ ID NO: 2146 and an AAV2 variant capsid. In some embodiments, the AAV particle comprises a viral genome comprising SEQ ID NO: 2146 and an AAV2/3 variant capsid. In some embodiments, the AAV particle comprises a viral genome comprising SEQ ID NO: 2146 and a capsid comprising SEQ ID NO: 1. In some embodiments, the AAV particle comprises a viral genome comprising SEQ ID NO: 2146 and a capsid encoded by a nucleic acid sequence comprising SEQ ID NO: 2. In some embodiments, the AAV particle comprises a viral genome comprising SEQ ID NO: 2146 and a capsid comprising SEQ ID NO: 4534. In some embodiments, the AAV particle comprises a viral genome comprising SEQ ID NO: 2146 and a capsid encoded by a nucleic acid sequence comprising SEQ ID NO: 3. In some embodiments, the AAV particle comprises a viral genome comprising SEQ ID NO: 2146 and a capsid comprising SEQ ID NO: 138. In some embodiments, the AAV particle comprises a viral genome comprising SEQ ID NO: 2146 and a capsid encoded by a nucleic acid sequence comprising SEQ ID NO: 137. In some embodiments, the AAV particle comprises a viral genome comprising SEQ ID NO: 2146 and a capsid comprising SEQ ID NO: 5. In some embodiments, the AAV particle comprises a viral genome comprising SEQ ID NO: 2146 and a capsid encoded by a nucleic acid sequence comprising SEQ ID NO: 6. In some embodiments, the AAV particle comprises a viral genome comprising SEQ ID NO: 2146 and a capsid comprising SEQ ID NO: 4. In some embodiments, the AAV particle comprises a viral genome comprising SEQ ID NO: 2146 and a capsid comprising SEQ ID NO: 11. In some embodiments, the AAV particle comprises a viral genome comprising SEQ ID NO: 2146 and a capsid comprising SEQ ID NO: 2679. In some embodiments, the AAV particle comprises a viral genome comprising SEQ ID NO: 2146 and a capsid comprising SEQ ID NO: 2809. In some embodiments, the AAV particle comprises a viral genome comprising SEQ ID NO: 2146 and a capsid comprising SEQ ID NO: 2871.

In some embodiments, the AAV particle comprises a viral genome comprising SEQ ID NO: 2147 and a VOY101 capsid. In some embodiments, the AAV particle comprises a viral genome comprising SEQ ID NO: 2147 and a VOY201 capsid. In some embodiments, the AAV particle comprises a viral genome comprising SEQ ID NO: 2147 and an AAV9 capsid. In some embodiments, the AAV particle comprises a viral genome comprising SEQ ID NO: 2147 and an AAV9K449R capsid. In some embodiments, the AAV particle comprises a viral genome comprising SEQ ID NO: 2147 and an AAVPHP.B capsid. In some embodiments, the AAV particle comprises a viral genome comprising SEQ ID NO: 2147 and an AAVPHP.N capsid. In some embodiments, the AAV particle comprises a viral genome comprising SEQ ID NO: 2147 and an AAV2 variant capsid. In some embodiments, the AAV particle comprises a viral genome comprising SEQ ID NO: 2147 and an AAV2/3 variant capsid. In some embodiments, the AAV particle comprises a viral genome comprising SEQ ID NO: 2147 and a capsid comprising SEQ ID NO: 1. In some embodiments, the AAV particle comprises a viral genome comprising SEQ ID NO: 2147 and a capsid encoded by a nucleic acid sequence comprising SEQ ID NO: 2. In some embodiments, the AAV particle comprises a viral genome comprising SEQ ID NO: 2147 and a capsid comprising SEQ ID NO: 4534. In some embodiments, the AAV particle comprises a viral genome comprising SEQ ID NO: 2147 and a capsid encoded by a nucleic acid sequence comprising SEQ ID NO: 3. In some embodiments, the AAV particle comprises a viral genome comprising SEQ ID NO: 2147 and a capsid comprising SEQ ID NO: 138. In some embodiments, the AAV particle comprises a viral genome comprising SEQ ID NO: 2147 and a capsid encoded by a nucleic acid sequence comprising SEQ ID NO: 137. In some embodiments, the AAV particle comprises a viral genome comprising SEQ ID NO: 2147 and a capsid comprising SEQ ID NO: 5. In some embodiments, the AAV particle comprises a viral genome comprising SEQ ID NO: 2147 and a capsid encoded by a nucleic acid sequence comprising SEQ ID NO: 6. In some embodiments, the AAV particle comprises a viral genome comprising SEQ ID NO: 2147 and a capsid comprising SEQ ID NO: 4. In some embodiments, the AAV particle comprises a viral genome comprising SEQ ID NO: 2147 and a capsid comprising SEQ ID NO: 11. In some embodiments, the AAV particle comprises a viral genome comprising SEQ ID NO: 2147 and a capsid comprising SEQ ID NO: 2679. In some embodiments, the AAV particle comprises a viral genome comprising SEQ ID NO: 2147 and a capsid comprising SEQ ID NO: 2809. In some embodiments, the AAV particle comprises a viral genome comprising SEQ ID NO: 2147 and a capsid comprising SEQ ID NO: 2871.

In some embodiments, the AAV particle comprises a viral genome comprising SEQ ID NO: 2148 and a VOY101 capsid. In some embodiments, the AAV particle comprises a viral genome comprising SEQ ID NO: 2148 and a VOY201 capsid. In some embodiments, the AAV particle comprises a viral genome comprising SEQ ID NO: 2148 and an AAV9 capsid. In some embodiments, the AAV particle comprises a viral genome comprising SEQ ID NO: 2148 and an AAV9K449R capsid. In some embodiments, the AAV particle comprises a viral genome comprising SEQ ID NO: 2148 and an AAVPHP.B capsid. In some embodiments, the AAV particle comprises a viral genome comprising SEQ ID NO: 2148 and an AAVPHP.N capsid. In some embodiments, the AAV particle comprises a viral genome comprising SEQ ID NO: 2148 and an AAV2 variant capsid. In some embodiments, the AAV particle comprises a viral genome comprising SEQ ID NO: 2148 and an AAV2/3 variant capsid. In some embodiments, the AAV particle comprises a viral genome comprising SEQ ID NO: 2148 and a capsid comprising SEQ ID NO: 1. In some embodiments, the AAV particle comprises a viral genome comprising SEQ ID NO: 2148 and a capsid encoded by a nucleic acid sequence comprising SEQ ID NO: 2. In some embodiments, the AAV particle comprises a viral genome comprising SEQ ID NO: 2148 and a capsid comprising SEQ ID NO: 4534. In some embodiments, the AAV particle comprises a viral genome comprising SEQ ID NO: 2148 and a capsid encoded by a nucleic acid sequence comprising SEQ ID NO: 3. In some embodiments, the AAV particle comprises a viral genome comprising SEQ ID NO: 2148 and a capsid comprising SEQ ID NO: 138. In some embodiments, the AAV particle comprises a viral genome comprising SEQ ID NO: 2148 and a capsid encoded by a nucleic acid sequence comprising SEQ ID NO: 137. In some embodiments, the AAV particle comprises a viral genome comprising SEQ ID NO: 2148 and a capsid comprising SEQ ID NO: 5. In some embodiments, the AAV particle comprises a viral genome comprising SEQ ID NO: 2148 and a capsid encoded by a nucleic acid sequence comprising SEQ ID NO: 6. In some embodiments, the AAV particle comprises a viral genome comprising SEQ ID NO: 2148 and a capsid comprising SEQ ID NO: 4. In some embodiments, the AAV particle comprises a viral genome comprising SEQ ID NO: 2148 and a capsid comprising SEQ ID NO: 11. In some embodiments, the AAV particle comprises a viral genome comprising SEQ ID NO: 2148 and a capsid comprising SEQ ID NO: 2679. In some embodiments, the AAV particle comprises a viral genome comprising SEQ ID NO: 2148 and a capsid comprising SEQ ID NO: 2809. In some embodiments, the AAV particle comprises a viral genome comprising SEQ ID NO: 2148 and a capsid comprising SEQ ID NO: 2871.

In some embodiments, the AAV particle comprises a viral genome comprising SEQ ID NO: 2149 and a VOY101 capsid. In some embodiments, the AAV particle comprises a viral genome comprising SEQ ID NO: 2149 and a VOY201 capsid. In some embodiments, the AAV particle comprises a viral genome comprising SEQ ID NO: 2149 and an AAV9 capsid. In some embodiments, the AAV particle comprises a viral genome comprising SEQ ID NO: 2149 and an AAV9K449R capsid. In some embodiments, the AAV particle comprises a viral genome comprising SEQ ID NO: 2149 and an AAVPHP.B capsid. In some embodiments, the AAV particle comprises a viral genome comprising SEQ ID NO: 2149 and an AAVPHP.N capsid. In some embodiments, the AAV particle comprises a viral genome comprising SEQ ID NO: 2149 and an AAV2 variant capsid. In some embodiments, the AAV particle comprises a viral genome comprising SEQ ID NO: 2149 and an AAV2/3 variant capsid. In some embodiments, the AAV particle comprises a viral genome comprising SEQ ID NO: 2149 and a capsid comprising SEQ ID NO: 1. In some embodiments, the AAV particle comprises a viral genome comprising SEQ ID NO: 2149 and a capsid encoded by a nucleic acid sequence comprising SEQ ID NO: 2. In some embodiments, the AAV particle comprises a viral genome comprising SEQ ID NO: 2149 and a capsid comprising SEQ ID NO: 4534. In some embodiments, the AAV particle comprises a viral genome comprising SEQ ID NO: 2149 and a capsid encoded by a nucleic acid sequence comprising SEQ ID NO: 3. In some embodiments, the AAV particle comprises a viral genome comprising SEQ ID NO: 2149 and a capsid comprising SEQ ID NO: 138. In some embodiments, the AAV particle comprises a viral genome comprising SEQ ID NO: 2149 and a capsid encoded by a nucleic acid sequence comprising SEQ ID NO: 137. In some embodiments, the AAV particle comprises a viral genome comprising SEQ ID NO: 2149 and a capsid comprising SEQ ID NO: 5. In some embodiments, the AAV particle comprises a viral genome comprising SEQ ID NO: 2149 and a capsid encoded by a nucleic acid sequence comprising SEQ ID NO: 6. In some embodiments, the AAV particle comprises a viral genome comprising SEQ ID NO: 2149 and a capsid comprising SEQ ID NO: 4. In some embodiments, the AAV particle comprises a viral genome comprising SEQ ID NO: 2149 and a capsid comprising SEQ ID NO: 11. In some embodiments, the AAV particle comprises a viral genome comprising SEQ ID NO: 2149 and a capsid comprising SEQ ID NO: 2679. In some embodiments, the AAV particle comprises a viral genome comprising SEQ ID NO: 2149 and a capsid comprising SEQ ID NO: 2809. In some embodiments, the AAV particle comprises a viral genome comprising SEQ ID NO: 2149 and a capsid comprising SEQ ID NO: 2871.

In some embodiments, the AAV particle comprises a viral genome comprising SEQ ID NO: 2150 and a VOY101 capsid. In some embodiments, the AAV particle comprises a viral genome comprising SEQ ID NO: 2150 and a VOY201 capsid. In some embodiments, the AAV particle comprises a viral genome comprising SEQ ID NO: 2150 and an AAV9 capsid. In some embodiments, the AAV particle comprises a viral genome comprising SEQ ID NO: 2150 and an AAV9K449R capsid. In some embodiments, the AAV particle comprises a viral genome comprising SEQ ID NO: 2150 and an AAVPHP.B capsid. In some embodiments, the AAV particle comprises a viral genome comprising SEQ ID NO: 2150 and an AAVPHP.N capsid. In some embodiments, the AAV particle comprises a viral genome comprising SEQ ID NO: 2150 and an AAV2 variant capsid. In some embodiments, the AAV particle comprises a viral genome comprising SEQ ID NO: 2150 and an AAV2/3 variant capsid. In some embodiments, the AAV particle comprises a viral genome comprising SEQ ID NO: 2150 and a capsid comprising SEQ ID NO: 1. In some embodiments, the AAV particle comprises a viral genome comprising SEQ ID NO: 2150 and a capsid encoded by a nucleic acid sequence comprising SEQ ID NO: 2. In some embodiments, the AAV particle comprises a viral genome comprising SEQ ID NO: 2150 and a capsid comprising SEQ ID NO: 4534. In some embodiments, the AAV particle comprises a viral genome comprising SEQ ID NO: 2150 and a capsid encoded by a nucleic acid sequence comprising SEQ ID NO: 3. In some embodiments, the AAV particle comprises a viral genome comprising SEQ ID NO: 2150 and a capsid comprising SEQ ID NO: 138. In some embodiments, the AAV particle comprises a viral genome comprising SEQ ID NO: 2150 and a capsid encoded by a nucleic acid sequence comprising SEQ ID NO: 137. In some embodiments, the AAV particle comprises a viral genome comprising SEQ ID NO: 2150 and a capsid comprising SEQ ID NO: 5. In some embodiments, the AAV particle comprises a viral genome comprising SEQ ID NO: 2150 and a capsid encoded by a nucleic acid sequence comprising SEQ ID NO: 6. In some embodiments, the AAV particle comprises a viral genome comprising SEQ ID NO: 2150 and a capsid comprising SEQ ID NO: 4. In some embodiments, the AAV particle comprises a viral genome comprising SEQ ID NO: 2150 and a capsid comprising SEQ ID NO: 11. In some embodiments, the AAV particle comprises a viral genome comprising SEQ ID NO: 2150 and a capsid comprising SEQ ID NO: 2679. In some embodiments, the AAV particle comprises a viral genome comprising SEQ ID NO: 2150 and a capsid comprising SEQ ID NO: 2809. In some embodiments, the AAV particle comprises a viral genome comprising SEQ ID NO: 2150 and a capsid comprising SEQ ID NO: 2871.

In some embodiments, the AAV particle comprises a viral genome comprising SEQ ID NO: 2151 and a VOY101 capsid. In some embodiments, the AAV particle comprises a viral genome comprising SEQ ID NO: 2151 and a VOY201 capsid. In some embodiments, the AAV particle comprises a viral genome comprising SEQ ID NO: 2151 and an AAV9 capsid. In some embodiments, the AAV particle comprises a viral genome comprising SEQ ID NO: 2151 and an AAV9K449R capsid. In some embodiments, the AAV particle comprises a viral genome comprising SEQ ID NO: 2151 and an AAVPHP.B capsid. In some embodiments, the AAV particle comprises a viral genome comprising SEQ ID NO: 2151 and an AAVPHP.N capsid. In some embodiments, the AAV particle comprises a viral genome comprising SEQ ID NO: 2151 and an AAV2 variant capsid. In some embodiments, the AAV particle comprises a viral genome comprising SEQ ID NO: 2151 and an AAV2/3 variant capsid. In some embodiments, the AAV particle comprises a viral genome comprising SEQ ID NO: 2151 and a capsid comprising SEQ ID NO: 1. In some embodiments, the AAV particle comprises a viral genome comprising SEQ ID NO: 2151 and a capsid encoded by a nucleic acid sequence comprising SEQ ID NO: 2. In some embodiments, the AAV particle comprises a viral genome comprising SEQ ID NO: 2151 and a capsid comprising SEQ ID NO: 4534. In some embodiments, the AAV particle comprises a viral genome comprising SEQ ID NO: 2151 and a capsid encoded by a nucleic acid sequence comprising SEQ ID NO: 3. In some embodiments, the AAV particle comprises a viral genome comprising SEQ ID NO: 2151 and a capsid comprising SEQ ID NO: 138. In some embodiments, the AAV particle comprises a viral genome comprising SEQ ID NO: 2151 and a capsid encoded by a nucleic acid sequence comprising SEQ ID NO: 137. In some embodiments, the AAV particle comprises a viral genome comprising SEQ ID NO: 2151 and a capsid comprising SEQ ID NO: 5. In some embodiments, the AAV particle comprises a viral genome comprising SEQ ID NO: 2151 and a capsid encoded by a nucleic acid sequence comprising SEQ ID NO: 6. In some embodiments, the AAV particle comprises a viral genome comprising SEQ ID NO: 2151 and a capsid comprising SEQ ID NO: 4. In some embodiments, the AAV particle comprises a viral genome comprising SEQ ID NO: 2151 and a capsid comprising SEQ ID NO: 11. In some embodiments, the AAV particle comprises a viral genome comprising SEQ ID NO: 2151 and a capsid comprising SEQ ID NO: 2679. In some embodiments, the AAV particle comprises a viral genome comprising SEQ ID NO: 2151 and a capsid comprising SEQ ID NO: 2809. In some embodiments, the AAV particle comprises a viral genome comprising SEQ ID NO: 2151 and a capsid comprising SEQ ID NO: 2871.

In some embodiments, the AAV particle comprises a viral genome comprising SEQ ID NO: 2152 and a VOY101 capsid. In some embodiments, the AAV particle comprises a viral genome comprising SEQ ID NO: 2152 and a VOY201 capsid. In some embodiments, the AAV particle comprises a viral genome comprising SEQ ID NO: 2152 and an AAV9 capsid. In some embodiments, the AAV particle comprises a viral genome comprising SEQ ID NO: 2152 and an AAV9K449R capsid. In some embodiments, the AAV particle comprises a viral genome comprising SEQ ID NO: 2152 and an AAVPHP.B capsid. In some embodiments, the AAV particle comprises a viral genome comprising SEQ ID NO: 2152 and an AAVPHP.N capsid. In some embodiments, the AAV particle comprises a viral genome comprising SEQ ID NO: 2152 and an AAV2 variant capsid. In some embodiments, the AAV particle comprises a viral genome comprising SEQ ID NO: 2152 and an AAV2/3 variant capsid. In some embodiments, the AAV particle comprises a viral genome comprising SEQ ID NO: 2152 and a capsid comprising SEQ ID NO: 1. In some embodiments, the AAV particle comprises a viral genome comprising SEQ ID NO: 2152 and a capsid encoded by a nucleic acid sequence comprising SEQ ID NO: 2. In some embodiments, the AAV particle comprises a viral genome comprising SEQ ID NO: 2152 and a capsid comprising SEQ ID NO: 4534. In some embodiments, the AAV particle comprises a viral genome comprising SEQ ID NO: 2152 and a capsid encoded by a nucleic acid sequence comprising SEQ ID NO: 3. In some embodiments, the AAV particle comprises a viral genome comprising SEQ ID NO: 2152 and a capsid comprising SEQ ID NO: 138. In some embodiments, the AAV particle comprises a viral genome comprising SEQ ID NO: 2152 and a capsid encoded by a nucleic acid sequence comprising SEQ ID NO: 137. In some embodiments, the AAV particle comprises a viral genome comprising SEQ ID NO: 2152 and a capsid comprising SEQ ID NO: 5. In some embodiments, the AAV particle comprises a viral genome comprising SEQ ID NO: 2152 and a capsid encoded by a nucleic acid sequence comprising SEQ ID NO: 6. In some embodiments, the AAV particle comprises a viral genome comprising SEQ ID NO: 2152 and a capsid comprising SEQ ID NO: 4. In some embodiments, the AAV particle comprises a viral genome comprising SEQ ID NO: 2152 and a capsid comprising SEQ ID NO: 11. In some embodiments, the AAV particle comprises a viral genome comprising SEQ ID NO: 2152 and a capsid comprising SEQ ID NO: 2679. In some embodiments, the AAV particle comprises a viral genome comprising SEQ ID NO: 2152 and a capsid comprising SEQ ID NO: 2809. In some embodiments, the AAV particle comprises a viral genome comprising SEQ ID NO: 2152 and a capsid comprising SEQ ID NO: 2871.

In some embodiments, the AAV particle comprises a viral genome comprising SEQ ID NO: 2153 and a VOY101 capsid. In some embodiments, the AAV particle comprises a viral genome comprising SEQ ID NO: 2153 and a VOY201 capsid. In some embodiments, the AAV particle comprises a viral genome comprising SEQ ID NO: 2153 and an AAV9 capsid. In some embodiments, the AAV particle comprises a viral genome comprising SEQ ID NO: 2153 and an AAV9K449R capsid. In some embodiments, the AAV particle comprises a viral genome comprising SEQ ID NO: 2153 and an AAVPHP.B capsid. In some embodiments, the AAV particle comprises a viral genome comprising SEQ ID NO: 2153 and an AAVPHP.N capsid. In some embodiments, the AAV particle comprises a viral genome comprising SEQ ID NO: 2153 and an AAV2 variant capsid. In some embodiments, the AAV particle comprises a viral genome comprising SEQ ID NO: 2153 and an AAV2/3 variant capsid. In some embodiments, the AAV particle comprises a viral genome comprising SEQ ID NO: 2153 and a capsid comprising SEQ ID NO: 1. In some embodiments, the AAV particle comprises a viral genome comprising SEQ ID NO: 2153 and a capsid encoded by a nucleic acid sequence comprising SEQ ID NO: 2. In some embodiments, the AAV particle comprises a viral genome comprising SEQ ID NO: 2153 and a capsid comprising SEQ ID NO: 4534. In some embodiments, the AAV particle comprises a viral genome comprising SEQ ID NO: 2153 and a capsid encoded by a nucleic acid sequence comprising SEQ ID NO: 3. In some embodiments, the AAV particle comprises a viral genome comprising SEQ ID NO: 2153 and a capsid comprising SEQ ID NO: 138. In some embodiments, the AAV particle comprises a viral genome comprising SEQ ID NO: 2153 and a capsid encoded by a nucleic acid sequence comprising SEQ ID NO: 137. In some embodiments, the AAV particle comprises a viral genome comprising SEQ ID NO: 2153 and a capsid comprising SEQ ID NO: 5. In some embodiments, the AAV particle comprises a viral genome comprising SEQ ID NO: 2153 and a capsid encoded by a nucleic acid sequence comprising SEQ ID NO: 6. In some embodiments, the AAV particle comprises a viral genome comprising SEQ ID NO: 2153 and a capsid comprising SEQ ID NO: 4. In some embodiments, the AAV particle comprises a viral genome comprising SEQ ID NO: 2153 and a capsid comprising SEQ ID NO: 11. In some embodiments, the AAV particle comprises a viral genome comprising SEQ ID NO: 2153 and a capsid comprising SEQ ID NO: 2679. In some embodiments, the AAV particle comprises a viral genome comprising SEQ ID NO: 2153 and a capsid comprising SEQ ID NO: 2809. In some embodiments, the AAV particle comprises a viral genome comprising SEQ ID NO: 2153 and a capsid comprising SEQ ID NO: 2871.

In some embodiments, the AAV particle comprises a viral genome comprising SEQ ID NO: 2154 and a VOY101 capsid. In some embodiments, the AAV particle comprises a viral genome comprising SEQ ID NO: 2154 and a VOY201 capsid. In some embodiments, the AAV particle comprises a viral genome comprising SEQ ID NO: 2154 and an AAV9 capsid. In some embodiments, the AAV particle comprises a viral genome comprising SEQ ID NO: 2154 and an AAV9K449R capsid. In some embodiments, the AAV particle comprises a viral genome comprising SEQ ID NO: 2154 and an AAVPHP.B capsid. In some embodiments, the AAV particle comprises a viral genome comprising SEQ ID NO: 2154 and an AAVPHP.N capsid. In some embodiments, the AAV particle comprises a viral genome comprising SEQ ID NO: 2154 and an AAV2 variant capsid. In some embodiments, the AAV particle comprises a viral genome comprising SEQ ID NO: 2154 and an AAV2/3 variant capsid. In some embodiments, the AAV particle comprises a viral genome comprising SEQ ID NO: 2154 and a capsid comprising SEQ ID NO: 1. In some embodiments, the AAV particle comprises a viral genome comprising SEQ ID NO: 2154 and a capsid encoded by a nucleic acid sequence comprising SEQ ID NO: 2. In some embodiments, the AAV particle comprises a viral genome comprising SEQ ID NO: 2154 and a capsid comprising SEQ ID NO: 4534. In some embodiments, the AAV particle comprises a viral genome comprising SEQ ID NO: 2154 and a capsid encoded by a nucleic acid sequence comprising SEQ ID NO: 3. In some embodiments, the AAV particle comprises a viral genome comprising SEQ ID NO: 2154 and a capsid comprising SEQ ID NO: 138. In some embodiments, the AAV particle comprises a viral genome comprising SEQ ID NO: 2154 and a capsid encoded by a nucleic acid sequence comprising SEQ ID NO: 137. In some embodiments, the AAV particle comprises a viral genome comprising SEQ ID NO: 2154 and a capsid comprising SEQ ID NO: 5. In some embodiments, the AAV particle comprises a viral genome comprising SEQ ID NO: 2154 and a capsid encoded by a nucleic acid sequence comprising SEQ ID NO: 6. In some embodiments, the AAV particle comprises a viral genome comprising SEQ ID NO: 2154 and a capsid comprising SEQ ID NO: 4. In some embodiments, the AAV particle comprises a viral genome comprising SEQ ID NO: 2154 and a capsid comprising SEQ ID NO: 11. In some embodiments, the AAV particle comprises a viral genome comprising SEQ ID NO: 2154 and a capsid comprising SEQ ID NO: 2679. In some embodiments, the AAV particle comprises a viral genome comprising SEQ ID NO: 2154 and a capsid comprising SEQ ID NO: 2809. In some embodiments, the AAV particle comprises a viral genome comprising SEQ ID NO: 2154 and a capsid comprising SEQ ID NO: 2871.

In some embodiments, the AAV particle comprises a viral genome comprising SEQ ID NO: 2155 and a VOY101 capsid. In some embodiments, the AAV particle comprises a viral genome comprising SEQ ID NO: 2155 and a VOY201 capsid. In some embodiments, the AAV particle comprises a viral genome comprising SEQ ID NO: 2155 and an AAV9 capsid. In some embodiments, the AAV particle comprises a viral genome comprising SEQ ID NO: 2155 and an AAV9K449R capsid. In some embodiments, the AAV particle comprises a viral genome comprising SEQ ID NO: 2155 and an AAVPHP.B capsid. In some embodiments, the AAV particle comprises a viral genome comprising SEQ ID NO: 2155 and an AAVPHP.N capsid. In some embodiments, the AAV particle comprises a viral genome comprising SEQ ID NO: 2155 and an AAV2 variant capsid. In some embodiments, the AAV particle comprises a viral genome comprising SEQ ID NO: 2155 and an AAV2/3 variant capsid. In some embodiments, the AAV particle comprises a viral genome comprising SEQ ID NO: 2155 and a capsid comprising SEQ ID NO: 1. In some embodiments, the AAV particle comprises a viral genome comprising SEQ ID NO: 2155 and a capsid encoded by a nucleic acid sequence comprising SEQ ID NO: 2. In some embodiments, the AAV particle comprises a viral genome comprising SEQ ID NO: 2155 and a capsid comprising SEQ ID NO: 4534. In some embodiments, the AAV particle comprises a viral genome comprising SEQ ID NO: 2155 and a capsid encoded by a nucleic acid sequence comprising SEQ ID NO: 3. In some embodiments, the AAV particle comprises a viral genome comprising SEQ ID NO: 2155 and a capsid comprising SEQ ID NO: 138. In some embodiments, the AAV particle comprises a viral genome comprising SEQ ID NO: 2155 and a capsid encoded by a nucleic acid sequence comprising SEQ ID NO: 137. In some embodiments, the AAV particle comprises a viral genome comprising SEQ ID NO: 2155 and a capsid comprising SEQ ID NO: 5. In some embodiments, the AAV particle comprises a viral genome comprising SEQ ID NO: 2155 and a capsid encoded by a nucleic acid sequence comprising SEQ ID NO: 6. In some embodiments, the AAV particle comprises a viral genome comprising SEQ ID NO: 2155 and a capsid comprising SEQ ID NO: 4. In some embodiments, the AAV particle comprises a viral genome comprising SEQ ID NO: 2155 and a capsid comprising SEQ ID NO: 11. In some embodiments, the AAV particle comprises a viral genome comprising SEQ ID NO: 2155 and a capsid comprising SEQ ID NO: 2679. In some embodiments, the AAV particle comprises a viral genome comprising SEQ ID NO: 2155 and a capsid comprising SEQ ID NO: 2809. In some embodiments, the AAV particle comprises a viral genome comprising SEQ ID NO: 2155 and a capsid comprising SEQ ID NO: 2871.

In some embodiments, the AAV particle comprises a viral genome comprising SEQ ID NO: 2156 and a VOY101 capsid. In some embodiments, the AAV particle comprises a viral genome comprising SEQ ID NO: 2156 and a VOY201 capsid. In some embodiments, the AAV particle comprises a viral genome comprising SEQ ID NO: 2156 and an AAV9 capsid. In some embodiments, the AAV particle comprises a viral genome comprising SEQ ID NO: 2156 and an AAV9K449R capsid. In some embodiments, the AAV particle comprises a viral genome comprising SEQ ID NO: 2156 and an AAVPHP.B capsid. In some embodiments, the AAV particle comprises a viral genome comprising SEQ ID NO: 2156 and an AAVPHP.N capsid. In some embodiments, the AAV particle comprises a viral genome comprising SEQ ID NO: 2156 and an AAV2 variant capsid. In some embodiments, the AAV particle comprises a viral genome comprising SEQ ID NO: 2156 and an AAV2/3 variant capsid. In some embodiments, the AAV particle comprises a viral genome comprising SEQ ID NO: 2156 and a capsid comprising SEQ ID NO: 1. In some embodiments, the AAV particle comprises a viral genome comprising SEQ ID NO: 2156 and a capsid encoded by a nucleic acid sequence comprising SEQ ID NO: 2. In some embodiments, the AAV particle comprises a viral genome comprising SEQ ID NO: 2156 and a capsid comprising SEQ ID NO: 4534. In some embodiments, the AAV particle comprises a viral genome comprising SEQ ID NO: 2156 and a capsid encoded by a nucleic acid sequence comprising SEQ ID NO: 3. In some embodiments, the AAV particle comprises a viral genome comprising SEQ ID NO: 2156 and a capsid comprising SEQ ID NO: 138. In some embodiments, the AAV particle comprises a viral genome comprising SEQ ID NO: 2156 and a capsid encoded by a nucleic acid sequence comprising SEQ ID NO: 137. In some embodiments, the AAV particle comprises a viral genome comprising SEQ ID NO: 2156 and a capsid comprising SEQ ID NO: 5. In some embodiments, the AAV particle comprises a viral genome comprising SEQ ID NO: 2156 and a capsid encoded by a nucleic acid sequence comprising SEQ ID NO: 6. In some embodiments, the AAV particle comprises a viral genome comprising SEQ ID NO: 2156 and a capsid comprising SEQ ID NO: 4. In some embodiments, the AAV particle comprises a viral genome comprising SEQ ID NO: 2156 and a capsid comprising SEQ ID NO: 11. In some embodiments, the AAV particle comprises a viral genome comprising SEQ ID NO: 2156 and a capsid comprising SEQ ID NO: 2679. In some embodiments, the AAV particle comprises a viral genome comprising SEQ ID NO: 2156 and a capsid comprising SEQ ID NO: 2809. In some embodiments, the AAV particle comprises a viral genome comprising SEQ ID NO: 2156 and a capsid comprising SEQ ID NO: 2871.

In some embodiments, the AAV particle comprises a viral genome comprising SEQ ID NO: 2157 and a VOY101 capsid. In some embodiments, the AAV particle comprises a viral genome comprising SEQ ID NO: 2157 and a VOY201 capsid. In some embodiments, the AAV particle comprises a viral genome comprising SEQ ID NO: 2157 and an AAV9 capsid. In some embodiments, the AAV particle comprises a viral genome comprising SEQ ID NO: 2157 and an AAV9K449R capsid. In some embodiments, the AAV particle comprises a viral genome comprising SEQ ID NO: 2157 and an AAVPHP.B capsid. In some embodiments, the AAV particle comprises a viral genome comprising SEQ ID NO: 2157 and an AAVPHP.N capsid. In some embodiments, the AAV particle comprises a viral genome comprising SEQ ID NO: 2157 and an AAV2 variant capsid. In some embodiments, the AAV particle comprises a viral genome comprising SEQ ID NO: 2157 and an AAV2/3 variant capsid. In some embodiments, the AAV particle comprises a viral genome comprising SEQ ID NO: 2157 and a capsid comprising SEQ ID NO: 1. In some embodiments, the AAV particle comprises a viral genome comprising SEQ ID NO: 2157 and a capsid encoded by a nucleic acid sequence comprising SEQ ID NO: 2. In some embodiments, the AAV particle comprises a viral genome comprising SEQ ID NO: 2157 and a capsid comprising SEQ ID NO: 4534. In some embodiments, the AAV particle comprises a viral genome comprising SEQ ID NO: 2157 and a capsid encoded by a nucleic acid sequence comprising SEQ ID NO: 3. In some embodiments, the AAV particle comprises a viral genome comprising SEQ ID NO: 2157 and a capsid comprising SEQ ID NO: 138. In some embodiments, the AAV particle comprises a viral genome comprising SEQ ID NO: 2157 and a capsid encoded by a nucleic acid sequence comprising SEQ ID NO: 137. In some embodiments, the AAV particle comprises a viral genome comprising SEQ ID NO: 2157 and a capsid comprising SEQ ID NO: 5. In some embodiments, the AAV particle comprises a viral genome comprising SEQ ID NO: 2157 and a capsid encoded by a nucleic acid sequence comprising SEQ ID NO: 6. In some embodiments, the AAV particle comprises a viral genome comprising SEQ ID NO: 2157 and a capsid comprising SEQ ID NO: 4. In some embodiments, the AAV particle comprises a viral genome comprising SEQ ID NO: 2157 and a capsid comprising SEQ ID NO: 11. In some embodiments, the AAV particle comprises a viral genome comprising SEQ ID NO: 2157 and a capsid comprising SEQ ID NO: 2679. In some embodiments, the AAV particle comprises a viral genome comprising SEQ ID NO: 2157 and a capsid comprising SEQ ID NO: 2809. In some embodiments, the AAV particle comprises a viral genome comprising SEQ ID NO: 2157 and a capsid comprising SEQ ID NO: 2871.

In some embodiments, the AAV particle comprises a viral genome comprising SEQ ID NO: 2158 and a VOY101 capsid. In some embodiments, the AAV particle comprises a viral genome comprising SEQ ID NO: 2158 and a VOY201 capsid. In some embodiments, the AAV particle comprises a viral genome comprising SEQ ID NO: 2158 and an AAV9 capsid. In some embodiments, the AAV particle comprises a viral genome comprising SEQ ID NO: 2158 and an AAV9K449R capsid. In some embodiments, the AAV particle comprises a viral genome comprising SEQ ID NO: 2158 and an AAVPHP.B capsid. In some embodiments, the AAV particle comprises a viral genome comprising SEQ ID NO: 2158 and an AAVPHP.N capsid. In some embodiments, the AAV particle comprises a viral genome comprising SEQ ID NO: 2158 and an AAV2 variant capsid. In some embodiments, the AAV particle comprises a viral genome comprising SEQ ID NO: 2158 and an AAV2/3 variant capsid. In some embodiments, the AAV particle comprises a viral genome comprising SEQ ID NO: 2158 and a capsid comprising SEQ ID NO: 1. In some embodiments, the AAV particle comprises a viral genome comprising SEQ ID NO: 2158 and a capsid encoded by a nucleic acid sequence comprising SEQ ID NO: 2. In some embodiments, the AAV particle comprises a viral genome comprising SEQ ID NO: 2158 and a capsid comprising SEQ ID NO: 4534. In some embodiments, the AAV particle comprises a viral genome comprising SEQ ID NO: 2158 and a capsid encoded by a nucleic acid sequence comprising SEQ ID NO: 3. In some embodiments, the AAV particle comprises a viral genome comprising SEQ ID NO: 2158 and a capsid comprising SEQ ID NO: 138. In some embodiments, the AAV particle comprises a viral genome comprising SEQ ID NO: 2158 and a capsid encoded by a nucleic acid sequence comprising SEQ ID NO: 137. In some embodiments, the AAV particle comprises a viral genome comprising SEQ ID NO: 2158 and a capsid comprising SEQ ID NO: 5. In some embodiments, the AAV particle comprises a viral genome comprising SEQ ID NO: 2158 and a capsid encoded by a nucleic acid sequence comprising SEQ ID NO: 6. In some embodiments, the AAV particle comprises a viral genome comprising SEQ ID NO: 2158 and a capsid comprising SEQ ID NO: 4. In some embodiments, the AAV particle comprises a viral genome comprising SEQ ID NO: 2158 and a capsid comprising SEQ ID NO: 11. In some embodiments, the AAV particle comprises a viral genome comprising SEQ ID NO: 2158 and a capsid comprising SEQ ID NO: 2679. In some embodiments, the AAV particle comprises a viral genome comprising SEQ ID NO: 2158 and a capsid comprising SEQ ID NO: 2809. In some embodiments, the AAV particle comprises a viral genome comprising SEQ ID NO: 2158 and a capsid comprising SEQ ID NO: 2871.

In some embodiments, the AAV particle comprises a viral genome comprising SEQ ID NO: 2159 and a VOY101 capsid. In some embodiments, the AAV particle comprises a viral genome comprising SEQ ID NO: 2159 and a VOY201 capsid. In some embodiments, the AAV particle comprises a viral genome comprising SEQ ID NO: 2159 and an AAV9 capsid. In some embodiments, the AAV particle comprises a viral genome comprising SEQ ID NO: 2159 and an AAV9K449R capsid. In some embodiments, the AAV particle comprises a viral genome comprising SEQ ID NO: 2159 and an AAVPHP.B capsid. In some embodiments, the AAV particle comprises a viral genome comprising SEQ ID NO: 2159 and an AAVPHP.N capsid. In some embodiments, the AAV particle comprises a viral genome comprising SEQ ID NO: 2159 and an AAV2 variant capsid. In some embodiments, the AAV particle comprises a viral genome comprising SEQ ID NO: 2159 and an AAV2/3 variant capsid. In some embodiments, the AAV particle comprises a viral genome comprising SEQ ID NO: 2159 and a capsid comprising SEQ ID NO: 1. In some embodiments, the AAV particle comprises a viral genome comprising SEQ ID NO: 2159 and a capsid encoded by a nucleic acid sequence comprising SEQ ID NO: 2. In some embodiments, the AAV particle comprises a viral genome comprising SEQ ID NO: 2159 and a capsid comprising SEQ ID NO: 4534. In some embodiments, the AAV particle comprises a viral genome comprising SEQ ID NO: 2159 and a capsid encoded by a nucleic acid sequence comprising SEQ ID NO: 3. In some embodiments, the AAV particle comprises a viral genome comprising SEQ ID NO: 2159 and a capsid comprising SEQ ID NO: 138. In some embodiments, the AAV particle comprises a viral genome comprising SEQ ID NO: 2159 and a capsid encoded by a nucleic acid sequence comprising SEQ ID NO: 137. In some embodiments, the AAV particle comprises a viral genome comprising SEQ ID NO: 2159 and a capsid comprising SEQ ID NO: 5. In some embodiments, the AAV particle comprises a viral genome comprising SEQ ID NO: 2159 and a capsid encoded by a nucleic acid sequence comprising SEQ ID NO: 6. In some embodiments, the AAV particle comprises a viral genome comprising SEQ ID NO: 2159 and a capsid comprising SEQ ID NO: 4. In some embodiments, the AAV particle comprises a viral genome comprising SEQ ID NO: 2159 and a capsid comprising SEQ ID NO: 11. In some embodiments, the AAV particle comprises a viral genome comprising SEQ ID NO: 2159 and a capsid comprising SEQ ID NO: 2679. In some embodiments, the AAV particle comprises a viral genome comprising SEQ ID NO: 2159 and a capsid comprising SEQ ID NO: 2809. In some embodiments, the AAV particle comprises a viral genome comprising SEQ ID NO: 2159 and a capsid comprising SEQ ID NO: 2871.

In some embodiments, the AAV particle comprises a viral genome comprising SEQ ID NO: 2160 and a VOY101 capsid. In some embodiments, the AAV particle comprises a viral genome comprising SEQ ID NO: 2160 and a VOY201 capsid. In some embodiments, the AAV particle comprises a viral genome comprising SEQ ID NO: 2160 and an AAV9 capsid. In some embodiments, the AAV particle comprises a viral genome comprising SEQ ID NO: 2160 and an AAV9K449R capsid. In some embodiments, the AAV particle comprises a viral genome comprising SEQ ID NO: 2160 and an AAVPHP.B capsid. In some embodiments, the AAV particle comprises a viral genome comprising SEQ ID NO: 2160 and an AAVPHP.N capsid. In some embodiments, the AAV particle comprises a viral genome comprising SEQ ID NO: 2160 and an AAV2 variant capsid. In some embodiments, the AAV particle comprises a viral genome comprising SEQ ID NO: 2160 and an AAV2/3 variant capsid. In some embodiments, the AAV particle comprises a viral genome comprising SEQ ID NO: 2160 and a capsid comprising SEQ ID NO: 1. In some embodiments, the AAV particle comprises a viral genome comprising SEQ ID NO: 2160 and a capsid encoded by a nucleic acid sequence comprising SEQ ID NO: 2. In some embodiments, the AAV particle comprises a viral genome comprising SEQ ID NO: 2160 and a capsid comprising SEQ ID NO: 4534. In some embodiments, the AAV particle comprises a viral genome comprising SEQ ID NO: 2160 and a capsid encoded by a nucleic acid sequence comprising SEQ ID NO: 3. In some embodiments, the AAV particle comprises a viral genome comprising SEQ ID NO: 2160 and a capsid comprising SEQ ID NO: 138. In some embodiments, the AAV particle comprises a viral genome comprising SEQ ID NO: 2160 and a capsid encoded by a nucleic acid sequence comprising SEQ ID NO: 137. In some embodiments, the AAV particle comprises a viral genome comprising SEQ ID NO: 2160 and a capsid comprising SEQ ID NO: 5. In some embodiments, the AAV particle comprises a viral genome comprising SEQ ID NO: 2160 and a capsid encoded by a nucleic acid sequence comprising SEQ ID NO: 6. In some embodiments, the AAV particle comprises a viral genome comprising SEQ ID NO: 2160 and a capsid comprising SEQ ID NO: 4. In some embodiments, the AAV particle comprises a viral genome comprising SEQ ID NO: 2160 and a capsid comprising SEQ ID NO: 11. In some embodiments, the AAV particle comprises a viral genome comprising SEQ ID NO: 2160 and a capsid comprising SEQ ID NO: 2679. In some embodiments, the AAV particle comprises a viral genome comprising SEQ ID NO: 2160 and a capsid comprising SEQ ID NO: 2809. In some embodiments, the AAV particle comprises a viral genome comprising SEQ ID NO: 2160 and a capsid comprising SEQ ID NO: 2871.

In some embodiments, the AAV particle comprises a viral genome comprising SEQ ID NO: 2161 and a VOY101 capsid. In some embodiments, the AAV particle comprises a viral genome comprising SEQ ID NO: 2161 and a VOY201 capsid. In some embodiments, the AAV particle comprises a viral genome comprising SEQ ID NO: 2161 and an AAV9 capsid. In some embodiments, the AAV particle comprises a viral genome comprising SEQ ID NO: 2161 and an AAV9K449R capsid. In some embodiments, the AAV particle comprises a viral genome comprising SEQ ID NO: 2161 and an AAVPHP.B capsid. In some embodiments, the AAV particle comprises a viral genome comprising SEQ ID NO: 2161 and an AAVPHP.N capsid. In some embodiments, the AAV particle comprises a viral genome comprising SEQ ID NO: 2161 and an AAV2 variant capsid. In some embodiments, the AAV particle comprises a viral genome comprising SEQ ID NO: 2161 and an AAV2/3 variant capsid. In some embodiments, the AAV particle comprises a viral genome comprising SEQ ID NO: 2161 and a capsid comprising SEQ ID NO: 1. In some embodiments, the AAV particle comprises a viral genome comprising SEQ ID NO: 2161 and a capsid encoded by a nucleic acid sequence comprising SEQ ID NO: 2. In some embodiments, the AAV particle comprises a viral genome comprising SEQ ID NO: 2161 and a capsid comprising SEQ ID NO: 4534. In some embodiments, the AAV particle comprises a viral genome comprising SEQ ID NO: 2161 and a capsid encoded by a nucleic acid sequence comprising SEQ ID NO: 3. In some embodiments, the AAV particle comprises a viral genome comprising SEQ ID NO: 2161 and a capsid comprising SEQ ID NO: 138. In some embodiments, the AAV particle comprises a viral genome comprising SEQ ID NO: 2161 and a capsid encoded by a nucleic acid sequence comprising SEQ ID NO: 137. In some embodiments, the AAV particle comprises a viral genome comprising SEQ ID NO: 2161 and a capsid comprising SEQ ID NO: 5. In some embodiments, the AAV particle comprises a viral genome comprising SEQ ID NO: 2161 and a capsid encoded by a nucleic acid sequence comprising SEQ ID NO: 6. In some embodiments, the AAV particle comprises a viral genome comprising SEQ ID NO: 2161 and a capsid comprising SEQ ID NO: 4. In some embodiments, the AAV particle comprises a viral genome comprising SEQ ID NO: 2161 and a capsid comprising SEQ ID NO: 11. In some embodiments, the AAV particle comprises a viral genome comprising SEQ ID NO: 2161 and a capsid comprising SEQ ID NO: 2679. In some embodiments, the AAV particle comprises a viral genome comprising SEQ ID NO: 2161 and a capsid comprising SEQ ID NO: 2809. In some embodiments, the AAV particle comprises a viral genome comprising SEQ ID NO: 2161 and a capsid comprising SEQ ID NO: 2871.

In some embodiments, the AAV particle comprises a viral genome comprising SEQ ID NO: 2162 and a VOY101 capsid. In some embodiments, the AAV particle comprises a viral genome comprising SEQ ID NO: 2162 and a VOY201 capsid. In some embodiments, the AAV particle comprises a viral genome comprising SEQ ID NO: 2162 and an AAV9 capsid. In some embodiments, the AAV particle comprises a viral genome comprising SEQ ID NO: 2162 and an AAV9K449R capsid. In some embodiments, the AAV particle comprises a viral genome comprising SEQ ID NO: 2162 and an AAVPHP.B capsid. In some embodiments, the AAV particle comprises a viral genome comprising SEQ ID NO: 2162 and an AAVPHP.N capsid. In some embodiments, the AAV particle comprises a viral genome comprising SEQ ID NO: 2162 and an AAV2 variant capsid. In some embodiments, the AAV particle comprises a viral genome comprising SEQ ID NO: 2162 and an AAV2/3 variant capsid. In some embodiments, the AAV particle comprises a viral genome comprising SEQ ID NO: 2162 and a capsid comprising SEQ ID NO: 1. In some embodiments, the AAV particle comprises a viral genome comprising SEQ ID NO: 2162 and a capsid encoded by a nucleic acid sequence comprising SEQ ID NO: 2. In some embodiments, the AAV particle comprises a viral genome comprising SEQ ID NO: 2162 and a capsid comprising SEQ ID NO: 4534. In some embodiments, the AAV particle comprises a viral genome comprising SEQ ID NO: 2162 and a capsid encoded by a nucleic acid sequence comprising SEQ ID NO: 3. In some embodiments, the AAV particle comprises a viral genome comprising SEQ ID NO: 2162 and a capsid comprising SEQ ID NO: 138. In some embodiments, the AAV particle comprises a viral genome comprising SEQ ID NO: 2162 and a capsid encoded by a nucleic acid sequence comprising SEQ ID NO: 137. In some embodiments, the AAV particle comprises a viral genome comprising SEQ ID NO: 2162 and a capsid comprising SEQ ID NO: 5. In some embodiments, the AAV particle comprises a viral genome comprising SEQ ID NO: 2162 and a capsid encoded by a nucleic acid sequence comprising SEQ ID NO: 6. In some embodiments, the AAV particle comprises a viral genome comprising SEQ ID NO: 2162 and a capsid comprising SEQ ID NO: 4. In some embodiments, the AAV particle comprises a viral genome comprising SEQ ID NO: 2162 and a capsid comprising SEQ ID NO: 11. In some embodiments, the AAV particle comprises a viral genome comprising SEQ ID NO: 2162 and a capsid comprising SEQ ID NO: 2679. In some embodiments, the AAV particle comprises a viral genome comprising SEQ ID NO: 2162 and a capsid comprising SEQ ID NO: 2809. In some embodiments, the AAV particle comprises a viral genome comprising SEQ ID NO: 2162 and a capsid comprising SEQ ID NO: 2871.

In some embodiments, the AAV particle comprises a viral genome comprising SEQ ID NO: 2163 and a VOY101 capsid. In some embodiments, the AAV particle comprises a viral genome comprising SEQ ID NO: 2163 and a VOY201 capsid. In some embodiments, the AAV particle comprises a viral genome comprising SEQ ID NO: 2163 and an AAV9 capsid. In some embodiments, the AAV particle comprises a viral genome comprising SEQ ID NO: 2163 and an AAV9K449R capsid. In some embodiments, the AAV particle comprises a viral genome comprising SEQ ID NO: 2163 and an AAVPHP.B capsid. In some embodiments, the AAV particle comprises a viral genome comprising SEQ ID NO: 2163 and an AAVPHP.N capsid. In some embodiments, the AAV particle comprises a viral genome comprising SEQ ID NO: 2163 and an AAV2 variant capsid. In some embodiments, the AAV particle comprises a viral genome comprising SEQ ID NO: 2163 and an AAV2/3 variant capsid. In some embodiments, the AAV particle comprises a viral genome comprising SEQ ID NO: 2163 and a capsid comprising SEQ ID NO: 1. In some embodiments, the AAV particle comprises a viral genome comprising SEQ ID NO: 2163 and a capsid encoded by a nucleic acid sequence comprising SEQ ID NO: 2. In some embodiments, the AAV particle comprises a viral genome comprising SEQ ID NO: 2163 and a capsid comprising SEQ ID NO: 4534. In some embodiments, the AAV particle comprises a viral genome comprising SEQ ID NO: 2163 and a capsid encoded by a nucleic acid sequence comprising SEQ ID NO: 3. In some embodiments, the AAV particle comprises a viral genome comprising SEQ ID NO: 2163 and a capsid comprising SEQ ID NO: 138. In some embodiments, the AAV particle comprises a viral genome comprising SEQ ID NO: 2163 and a capsid encoded by a nucleic acid sequence comprising SEQ ID NO: 137. In some embodiments, the AAV particle comprises a viral genome comprising SEQ ID NO: 2163 and a capsid comprising SEQ ID NO: 5. In some embodiments, the AAV particle comprises a viral genome comprising SEQ ID NO: 2163 and a capsid encoded by a nucleic acid sequence comprising SEQ ID NO: 6. In some embodiments, the AAV particle comprises a viral genome comprising SEQ ID NO: 2163 and a capsid comprising SEQ ID NO: 4. In some embodiments, the AAV particle comprises a viral genome comprising SEQ ID NO: 2163 and a capsid comprising SEQ ID NO: 11. In some embodiments, the AAV particle comprises a viral genome comprising SEQ ID NO: 2163 and a capsid comprising SEQ ID NO: 2679. In some embodiments, the AAV particle comprises a viral genome comprising SEQ ID NO: 2163 and a capsid comprising SEQ ID NO: 2809. In some embodiments, the AAV particle comprises a viral genome comprising SEQ ID NO: 2163 and a capsid comprising SEQ ID NO: 2871.

In some embodiments, the AAV particle comprises a viral genome comprising SEQ ID NO: 2164 and a VOY101 capsid. In some embodiments, the AAV particle comprises a viral genome comprising SEQ ID NO: 2164 and a VOY201 capsid. In some embodiments, the AAV particle comprises a viral genome comprising SEQ ID NO: 2164 and an AAV9 capsid. In some embodiments, the AAV particle comprises a viral genome comprising SEQ ID NO: 2164 and an AAV9K449R capsid. In some embodiments, the AAV particle comprises a viral genome comprising SEQ ID NO: 2164 and an AAVPHP.B capsid. In some embodiments, the AAV particle comprises a viral genome comprising SEQ ID NO: 2164 and an AAVPHP.N capsid. In some embodiments, the AAV particle comprises a viral genome comprising SEQ ID NO: 2164 and an AAV2 variant capsid. In some embodiments, the AAV particle comprises a viral genome comprising SEQ ID NO: 2164 and an AAV2/3 variant capsid. In some embodiments, the AAV particle comprises a viral genome comprising SEQ ID NO: 2164 and a capsid comprising SEQ ID NO: 1. In some embodiments, the AAV particle comprises a viral genome comprising SEQ ID NO: 2164 and a capsid encoded by a nucleic acid sequence comprising SEQ ID NO: 2. In some embodiments, the AAV particle comprises a viral genome comprising SEQ ID NO: 2164 and a capsid comprising SEQ ID NO: 4534. In some embodiments, the AAV particle comprises a viral genome comprising SEQ ID NO: 2164 and a capsid encoded by a nucleic acid sequence comprising SEQ ID NO: 3. In some embodiments, the AAV particle comprises a viral genome comprising SEQ ID NO: 2164 and a capsid comprising SEQ ID NO: 138. In some embodiments, the AAV particle comprises a viral genome comprising SEQ ID NO: 2164 and a capsid encoded by a nucleic acid sequence comprising SEQ ID NO: 137. In some embodiments, the AAV particle comprises a viral genome comprising SEQ ID NO: 2164 and a capsid comprising SEQ ID NO: 5. In some embodiments, the AAV particle comprises a viral genome comprising SEQ ID NO: 2164 and a capsid encoded by a nucleic acid sequence comprising SEQ ID NO: 6. In some embodiments, the AAV particle comprises a viral genome comprising SEQ ID NO: 2164 and a capsid comprising SEQ ID NO: 4. In some embodiments, the AAV particle comprises a viral genome comprising SEQ ID NO: 2164 and a capsid comprising SEQ ID NO: 11. In some embodiments, the AAV particle comprises a viral genome comprising SEQ ID NO: 2164 and a capsid comprising SEQ ID NO: 2679. In some embodiments, the AAV particle comprises a viral genome comprising SEQ ID NO: 2164 and a capsid comprising SEQ ID NO: 2809. In some embodiments, the AAV particle comprises a viral genome comprising SEQ ID NO: 2164 and a capsid comprising SEQ ID NO: 2871.

In some embodiments, the AAV particle comprises a viral genome comprising SEQ ID NO: 2165 and a VOY101 capsid. In some embodiments, the AAV particle comprises a viral genome comprising SEQ ID NO: 2165 and a VOY201 capsid. In some embodiments, the AAV particle comprises a viral genome comprising SEQ ID NO: 2165 and an AAV9 capsid. In some embodiments, the AAV particle comprises a viral genome comprising SEQ ID NO: 2165 and an AAV9K449R capsid. In some embodiments, the AAV particle comprises a viral genome comprising SEQ ID NO: 2165 and an AAVPHP.B capsid. In some embodiments, the AAV particle comprises a viral genome comprising SEQ ID NO: 2165 and an AAVPHP.N capsid. In some embodiments, the AAV particle comprises a viral genome comprising SEQ ID NO: 2165 and an AAV2 variant capsid. In some embodiments, the AAV particle comprises a viral genome comprising SEQ ID NO: 2165 and an AAV2/3 variant capsid. In some embodiments, the AAV particle comprises a viral genome comprising SEQ ID NO: 2165 and a capsid comprising SEQ ID NO: 1. In some embodiments, the AAV particle comprises a viral genome comprising SEQ ID NO: 2165 and a capsid encoded by a nucleic acid sequence comprising SEQ ID NO: 2. In some embodiments, the AAV particle comprises a viral genome comprising SEQ ID NO: 2165 and a capsid comprising SEQ ID NO: 4534. In some embodiments, the AAV particle comprises a viral genome comprising SEQ ID NO: 2165 and a capsid encoded by a nucleic acid sequence comprising SEQ ID NO: 3. In some embodiments, the AAV particle comprises a viral genome comprising SEQ ID NO: 2165 and a capsid comprising SEQ ID NO: 138. In some embodiments, the AAV particle comprises a viral genome comprising SEQ ID NO: 2165 and a capsid encoded by a nucleic acid sequence comprising SEQ ID NO: 137. In some embodiments, the AAV particle comprises a viral genome comprising SEQ ID NO: 2165 and a capsid comprising SEQ ID NO: 5. In some embodiments, the AAV particle comprises a viral genome comprising SEQ ID NO: 2165 and a capsid encoded by a nucleic acid sequence comprising SEQ ID NO: 6. In some embodiments, the AAV particle comprises a viral genome comprising SEQ ID NO: 2165 and a capsid comprising SEQ ID NO: 4. In some embodiments, the AAV particle comprises a viral genome comprising SEQ ID NO: 2165 and a capsid comprising SEQ ID NO: 11. In some embodiments, the AAV particle comprises a viral genome comprising SEQ ID NO: 2165 and a capsid comprising SEQ ID NO: 2679. In some embodiments, the AAV particle comprises a viral genome comprising SEQ ID NO: 2165 and a capsid comprising SEQ ID NO: 2809. In some embodiments, the AAV particle comprises a viral genome comprising SEQ ID NO: 2165 and a capsid comprising SEQ ID NO: 2871.

In some embodiments, the AAV particle comprises a viral genome comprising SEQ ID NO: 2166 and a VOY101 capsid. In some embodiments, the AAV particle comprises a viral genome comprising SEQ ID NO: 2166 and a VOY201 capsid. In some embodiments, the AAV particle comprises a viral genome comprising SEQ ID NO: 2166 and an AAV9 capsid. In some embodiments, the AAV particle comprises a viral genome comprising SEQ ID NO: 2166 and an AAV9K449R capsid. In some embodiments, the AAV particle comprises a viral genome comprising SEQ ID NO: 2166 and an AAVPHP.B capsid. In some embodiments, the AAV particle comprises a viral genome comprising SEQ ID NO: 2166 and an AAVPHP.N capsid. In some embodiments, the AAV particle comprises a viral genome comprising SEQ ID NO: 2166 and an AAV2 variant capsid. In some embodiments, the AAV particle comprises a viral genome comprising SEQ ID NO: 2166 and an AAV2/3 variant capsid. In some embodiments, the AAV particle comprises a viral genome comprising SEQ ID NO: 2166 and a capsid comprising SEQ ID NO: 1. In some embodiments, the AAV particle comprises a viral genome comprising SEQ ID NO: 2166 and a capsid encoded by a nucleic acid sequence comprising SEQ ID NO: 2. In some embodiments, the AAV particle comprises a viral genome comprising SEQ ID NO: 2166 and a capsid comprising SEQ ID NO: 4534. In some embodiments, the AAV particle comprises a viral genome comprising SEQ ID NO: 2166 and a capsid encoded by a nucleic acid sequence comprising SEQ ID NO: 3. In some embodiments, the AAV particle comprises a viral genome comprising SEQ ID NO: 2166 and a capsid comprising SEQ ID NO: 138. In some embodiments, the AAV particle comprises a viral genome comprising SEQ ID NO: 2166 and a capsid encoded by a nucleic acid sequence comprising SEQ ID NO: 137. In some embodiments, the AAV particle comprises a viral genome comprising SEQ ID NO: 2166 and a capsid comprising SEQ ID NO: 5. In some embodiments, the AAV particle comprises a viral genome comprising SEQ ID NO: 2166 and a capsid encoded by a nucleic acid sequence comprising SEQ ID NO: 6. In some embodiments, the AAV particle comprises a viral genome comprising SEQ ID NO: 2166 and a capsid comprising SEQ ID NO: 4. In some embodiments, the AAV particle comprises a viral genome comprising SEQ ID NO: 2166 and a capsid comprising SEQ ID NO: 11. In some embodiments, the AAV particle comprises a viral genome comprising SEQ ID NO: 2166 and a capsid comprising SEQ ID NO: 2679. In some embodiments, the AAV particle comprises a viral genome comprising SEQ ID NO: 2166 and a capsid comprising SEQ ID NO: 2809. In some embodiments, the AAV particle comprises a viral genome comprising SEQ ID NO: 2166 and a capsid comprising SEQ ID NO: 2871.

In some embodiments, the AAV particle comprises a viral genome comprising SEQ ID NO: 2167 and a VOY101 capsid. In some embodiments, the AAV particle comprises a viral genome comprising SEQ ID NO: 2167 and a VOY201 capsid. In some embodiments, the AAV particle comprises a viral genome comprising SEQ ID NO: 2167 and an AAV9 capsid. In some embodiments, the AAV particle comprises a viral genome comprising SEQ ID NO: 2167 and an AAV9K449R capsid. In some embodiments, the AAV particle comprises a viral genome comprising SEQ ID NO: 2167 and an AAVPHP.B capsid. In some embodiments, the AAV particle comprises a viral genome comprising SEQ ID NO: 2167 and an AAVPHP.N capsid. In some embodiments, the AAV particle comprises a viral genome comprising SEQ ID NO: 2167 and an AAV2 variant capsid. In some embodiments, the AAV particle comprises a viral genome comprising SEQ ID NO: 2167 and an AAV2/3 variant capsid. In some embodiments, the AAV particle comprises a viral genome comprising SEQ ID NO: 2167 and a capsid comprising SEQ ID NO: 1. In some embodiments, the AAV particle comprises a viral genome comprising SEQ ID NO: 2167 and a capsid encoded by a nucleic acid sequence comprising SEQ ID NO: 2. In some embodiments, the AAV particle comprises a viral genome comprising SEQ ID NO: 2167 and a capsid comprising SEQ ID NO: 4534. In some embodiments, the AAV particle comprises a viral genome comprising SEQ ID NO: 2167 and a capsid encoded by a nucleic acid sequence comprising SEQ ID NO: 3. In some embodiments, the AAV particle comprises a viral genome comprising SEQ ID NO: 2167 and a capsid comprising SEQ ID NO: 138. In some embodiments, the AAV particle comprises a viral genome comprising SEQ ID NO: 2167 and a capsid encoded by a nucleic acid sequence comprising SEQ ID NO: 137. In some embodiments, the AAV particle comprises a viral genome comprising SEQ ID NO: 2167 and a capsid comprising SEQ ID NO: 5. In some embodiments, the AAV particle comprises a viral genome comprising SEQ ID NO: 2167 and a capsid encoded by a nucleic acid sequence comprising SEQ ID NO: 6. In some embodiments, the AAV particle comprises a viral genome comprising SEQ ID NO: 2167 and a capsid comprising SEQ ID NO: 4. In some embodiments, the AAV particle comprises a viral genome comprising SEQ ID NO: 2167 and a capsid comprising SEQ ID NO: 11. In some embodiments, the AAV particle comprises a viral genome comprising SEQ ID NO: 2167 and a capsid comprising SEQ ID NO: 2679. In some embodiments, the AAV particle comprises a viral genome comprising SEQ ID NO: 2167 and a capsid comprising SEQ ID NO: 2809. In some embodiments, the AAV particle comprises a viral genome comprising SEQ ID NO: 2167 and a capsid comprising SEQ ID NO: 2871.

In some embodiments, the AAV particle comprises a viral genome comprising SEQ ID NO: 2168 and a VOY101 capsid. In some embodiments, the AAV particle comprises a viral genome comprising SEQ ID NO: 2168 and a VOY201 capsid. In some embodiments, the AAV particle comprises a viral genome comprising SEQ ID NO: 2168 and an AAV9 capsid. In some embodiments, the AAV particle comprises a viral genome comprising SEQ ID NO: 2168 and an AAV9K449R capsid. In some embodiments, the AAV particle comprises a viral genome comprising SEQ ID NO: 2168 and an AAVPHP.B capsid. In some embodiments, the AAV particle comprises a viral genome comprising SEQ ID NO: 2168 and an AAVPHP.N capsid. In some embodiments, the AAV particle comprises a viral genome comprising SEQ ID NO: 2168 and an AAV2 variant capsid. In some embodiments, the AAV particle comprises a viral genome comprising SEQ ID NO: 2168 and an AAV2/3 variant capsid. In some embodiments, the AAV particle comprises a viral genome comprising SEQ ID NO: 2168 and a capsid comprising SEQ ID NO: 1. In some embodiments, the AAV particle comprises a viral genome comprising SEQ ID NO: 2168 and a capsid encoded by a nucleic acid sequence comprising SEQ ID NO: 2. In some embodiments, the AAV particle comprises a viral genome comprising SEQ ID NO: 2168 and a capsid comprising SEQ ID NO: 4534. In some embodiments, the AAV particle comprises a viral genome comprising SEQ ID NO: 2168 and a capsid encoded by a nucleic acid sequence comprising SEQ ID NO: 3. In some embodiments, the AAV particle comprises a viral genome comprising SEQ ID NO: 2168 and a capsid comprising SEQ ID NO: 138. In some embodiments, the AAV particle comprises a viral genome comprising SEQ ID NO: 2168 and a capsid encoded by a nucleic acid sequence comprising SEQ ID NO: 137. In some embodiments, the AAV particle comprises a viral genome comprising SEQ ID NO: 2168 and a capsid comprising SEQ ID NO: 5. In some embodiments, the AAV particle comprises a viral genome comprising SEQ ID NO: 2168 and a capsid encoded by a nucleic acid sequence comprising SEQ ID NO: 6. In some embodiments, the AAV particle comprises a viral genome comprising SEQ ID NO: 2168 and a capsid comprising SEQ ID NO: 4. In some embodiments, the AAV particle comprises a viral genome comprising SEQ ID NO: 2168 and a capsid comprising SEQ ID NO: 11. In some embodiments, the AAV particle comprises a viral genome comprising SEQ ID NO: 2168 and a capsid comprising SEQ ID NO: 2679. In some embodiments, the AAV particle comprises a viral genome comprising SEQ ID NO: 2168 and a capsid comprising SEQ ID NO: 2809. In some embodiments, the AAV particle comprises a viral genome comprising SEQ ID NO: 2168 and a capsid comprising SEQ ID NO: 2871.

In some embodiments, the AAV particle comprises a viral genome comprising SEQ ID NO: 2171 and a VOY101 capsid. In some embodiments, the AAV particle comprises a viral genome comprising SEQ ID NO: 2171 and a VOY201 capsid. In some embodiments, the AAV particle comprises a viral genome comprising SEQ ID NO: 2171 and an AAV9 capsid. In some embodiments, the AAV particle comprises a viral genome comprising SEQ ID NO: 2171 and an AAV9K449R capsid. In some embodiments, the AAV particle comprises a viral genome comprising SEQ ID NO: 2171 and an AAVPHP.B capsid. In some embodiments, the AAV particle comprises a viral genome comprising SEQ ID NO: 2171 and an AAVPHP.N capsid. In some embodiments, the AAV particle comprises a viral genome comprising SEQ ID NO: 2171 and an AAV2 variant capsid. In some embodiments, the AAV particle comprises a viral genome comprising SEQ ID NO: 2171 and an AAV2/3 variant capsid. In some embodiments, the AAV particle comprises a viral genome comprising SEQ ID NO: 2171 and a capsid comprising SEQ ID NO: 1. In some embodiments, the AAV particle comprises a viral genome comprising SEQ ID NO: 2171 and a capsid encoded by a nucleic acid sequence comprising SEQ ID NO: 2. In some embodiments, the AAV particle comprises a viral genome comprising SEQ ID NO: 2171 and a capsid comprising SEQ ID NO: 4534. In some embodiments, the AAV particle comprises a viral genome comprising SEQ ID NO: 2171 and a capsid encoded by a nucleic acid sequence comprising SEQ ID NO: 3. In some embodiments, the AAV particle comprises a viral genome comprising SEQ ID NO: 2171 and a capsid comprising SEQ ID NO: 138. In some embodiments, the AAV particle comprises a viral genome comprising SEQ ID NO: 2171 and a capsid encoded by a nucleic acid sequence comprising SEQ ID NO: 137. In some embodiments, the AAV particle comprises a viral genome comprising SEQ ID NO: 2171 and a capsid comprising SEQ ID NO: 5. In some embodiments, the AAV particle comprises a viral genome comprising SEQ ID NO: 2171 and a capsid encoded by a nucleic acid sequence comprising SEQ ID NO: 6. In some embodiments, the AAV particle comprises a viral genome comprising SEQ ID NO: 2171 and a capsid comprising SEQ ID NO: 4. In some embodiments, the AAV particle comprises a viral genome comprising SEQ ID NO: 2171 and a capsid comprising SEQ ID NO: 11. In some embodiments, the AAV particle comprises a viral genome comprising SEQ ID NO: 2171 and a capsid comprising SEQ ID NO: 2679. In some embodiments, the AAV particle comprises a viral genome comprising SEQ ID NO: 2171 and a capsid comprising SEQ ID NO: 2809. In some embodiments, the AAV particle comprises a viral genome comprising SEQ ID NO: 2171 and a capsid comprising SEQ ID NO: 2871.

In some embodiments, the AAV particle comprises a viral genome comprising SEQ ID NO: 2172 and a VOY101 capsid. In some embodiments, the AAV particle comprises a viral genome comprising SEQ ID NO: 2172 and a VOY201 capsid. In some embodiments, the AAV particle comprises a viral genome comprising SEQ ID NO: 2172 and an AAV9 capsid. In some embodiments, the AAV particle comprises a viral genome comprising SEQ ID NO: 2172 and an AAV9K449R capsid. In some embodiments, the AAV particle comprises a viral genome comprising SEQ ID NO: 2172 and an AAVPHP.B capsid. In some embodiments, the AAV particle comprises a viral genome comprising SEQ ID NO: 2172 and an AAVPHP.N capsid. In some embodiments, the AAV particle comprises a viral genome comprising SEQ ID NO: 2172 and an AAV2 variant capsid. In some embodiments, the AAV particle comprises a viral genome comprising SEQ ID NO: 2172 and an AAV2/3 variant capsid. In some embodiments, the AAV particle comprises a viral genome comprising SEQ ID NO: 2172 and a capsid comprising SEQ ID NO: 1. In some embodiments, the AAV particle comprises a viral genome comprising SEQ ID NO: 2172 and a capsid encoded by a nucleic acid sequence comprising SEQ ID NO: 2. In some embodiments, the AAV particle comprises a viral genome comprising SEQ ID NO: 2172 and a capsid comprising SEQ ID NO: 4534. In some embodiments, the AAV particle comprises a viral genome comprising SEQ ID NO: 2172 and a capsid encoded by a nucleic acid sequence comprising SEQ ID NO: 3. In some embodiments, the AAV particle comprises a viral genome comprising SEQ ID NO: 2172 and a capsid comprising SEQ ID NO: 138. In some embodiments, the AAV particle comprises a viral genome comprising SEQ ID NO: 2172 and a capsid encoded by a nucleic acid sequence comprising SEQ ID NO: 137. In some embodiments, the AAV particle comprises a viral genome comprising SEQ ID NO: 2172 and a capsid comprising SEQ ID NO: 5. In some embodiments, the AAV particle comprises a viral genome comprising SEQ ID NO: 2172 and a capsid encoded by a nucleic acid sequence comprising SEQ ID NO: 6. In some embodiments, the AAV particle comprises a viral genome comprising SEQ ID NO: 2172 and a capsid comprising SEQ ID NO: 4. In some embodiments, the AAV particle comprises a viral genome comprising SEQ ID NO: 2172 and a capsid comprising SEQ ID NO: 11. In some embodiments, the AAV particle comprises a viral genome comprising SEQ ID NO: 2172 and a capsid comprising SEQ ID NO: 2679. In some embodiments, the AAV particle comprises a viral genome comprising SEQ ID NO: 2172 and a capsid comprising SEQ ID NO: 2809. In some embodiments, the AAV particle comprises a viral genome comprising SEQ ID NO: 2172 and a capsid comprising SEQ ID NO: 2871.

In some embodiments, the AAV particle comprises a viral genome comprising SEQ ID NO: 2173 and a VOY101 capsid. In some embodiments, the AAV particle comprises a viral genome comprising SEQ ID NO: 2173 and a VOY201 capsid. In some embodiments, the AAV particle comprises a viral genome comprising SEQ ID NO: 2173 and an AAV9 capsid. In some embodiments, the AAV particle comprises a viral genome comprising SEQ ID NO: 2173 and an AAV9K449R capsid. In some embodiments, the AAV particle comprises a viral genome comprising SEQ ID NO: 2173 and an AAVPHP.B capsid. In some embodiments, the AAV particle comprises a viral genome comprising SEQ ID NO: 2173 and an AAVPHP.N capsid. In some embodiments, the AAV particle comprises a viral genome comprising SEQ ID NO: 2173 and an AAV2 variant capsid. In some embodiments, the AAV particle comprises a viral genome comprising SEQ ID NO: 2173 and an AAV2/3 variant capsid. In some embodiments, the AAV particle comprises a viral genome comprising SEQ ID NO: 2173 and a capsid comprising SEQ ID NO: 1. In some embodiments, the AAV particle comprises a viral genome comprising SEQ ID NO: 2173 and a capsid encoded by a nucleic acid sequence comprising SEQ ID NO: 2. In some embodiments, the AAV particle comprises a viral genome comprising SEQ ID NO: 2173 and a capsid comprising SEQ ID NO: 4534. In some embodiments, the AAV particle comprises a viral genome comprising SEQ ID NO: 2173 and a capsid encoded by a nucleic acid sequence comprising SEQ ID NO: 3. In some embodiments, the AAV particle comprises a viral genome comprising SEQ ID NO: 2173 and a capsid comprising SEQ ID NO: 138. In some embodiments, the AAV particle comprises a viral genome comprising SEQ ID NO: 2173 and a capsid encoded by a nucleic acid sequence comprising SEQ ID NO: 137. In some embodiments, the AAV particle comprises a viral genome comprising SEQ ID NO: 2173 and a capsid comprising SEQ ID NO: 5. In some embodiments, the AAV particle comprises a viral genome comprising SEQ ID NO: 2173 and a capsid encoded by a nucleic acid sequence comprising SEQ ID NO: 6. In some embodiments, the AAV particle comprises a viral genome comprising SEQ ID NO: 2173 and a capsid comprising SEQ ID NO: 4. In some embodiments, the AAV particle comprises a viral genome comprising SEQ ID NO: 2173 and a capsid comprising SEQ ID NO: 11. In some embodiments, the AAV particle comprises a viral genome comprising SEQ ID NO: 2173 and a capsid comprising SEQ ID NO: 2679. In some embodiments, the AAV particle comprises a viral genome comprising SEQ ID NO: 2173 and a capsid comprising SEQ ID NO: 2809. In some embodiments, the AAV particle comprises a viral genome comprising SEQ ID NO: 2173 and a capsid comprising SEQ ID NO: 2871.

In some embodiments, the AAV particle comprises a viral genome comprising SEQ ID NO: 2174 and a VOY101 capsid. In some embodiments, the AAV particle comprises a viral genome comprising SEQ ID NO: 2174 and a VOY201 capsid. In some embodiments, the AAV particle comprises a viral genome comprising SEQ ID NO: 2174 and an AAV9 capsid. In some embodiments, the AAV particle comprises a viral genome comprising SEQ ID NO: 2174 and an AAV9K449R capsid. In some embodiments, the AAV particle comprises a viral genome comprising SEQ ID NO: 2174 and an AAVPHP.B capsid. In some embodiments, the AAV particle comprises a viral genome comprising SEQ ID NO: 2174 and an AAVPHP.N capsid. In some embodiments, the AAV particle comprises a viral genome comprising SEQ ID NO: 2174 and an AAV2 variant capsid. In some embodiments, the AAV particle comprises a viral genome comprising SEQ ID NO: 2174 and an AAV2/3 variant capsid. In some embodiments, the AAV particle comprises a viral genome comprising SEQ ID NO: 2174 and a capsid comprising SEQ ID NO: 1. In some embodiments, the AAV particle comprises a viral genome comprising SEQ ID NO: 2174 and a capsid encoded by a nucleic acid sequence comprising SEQ ID NO: 2. In some embodiments, the AAV particle comprises a viral genome comprising SEQ ID NO: 2174 and a capsid comprising SEQ ID NO: 4534. In some embodiments, the AAV particle comprises a viral genome comprising SEQ ID NO: 2174 and a capsid encoded by a nucleic acid sequence comprising SEQ ID NO: 3. In some embodiments, the AAV particle comprises a viral genome comprising SEQ ID NO: 2174 and a capsid comprising SEQ ID NO: 138. In some embodiments, the AAV particle comprises a viral genome comprising SEQ ID NO: 2174 and a capsid encoded by a nucleic acid sequence comprising SEQ ID NO: 137. In some embodiments, the AAV particle comprises a viral genome comprising SEQ ID NO: 2174 and a capsid comprising SEQ ID NO: 5. In some embodiments, the AAV particle comprises a viral genome comprising SEQ ID NO: 2174 and a capsid encoded by a nucleic acid sequence comprising SEQ ID NO: 6. In some embodiments, the AAV particle comprises a viral genome comprising SEQ ID NO: 2174 and a capsid comprising SEQ ID NO: 4. In some embodiments, the AAV particle comprises a viral genome comprising SEQ ID NO: 2174 and a capsid comprising SEQ ID NO: 11. In some embodiments, the AAV particle comprises a viral genome comprising SEQ ID NO: 2174 and a capsid comprising SEQ ID NO: 2679. In some embodiments, the AAV particle comprises a viral genome comprising SEQ ID NO: 2174 and a capsid comprising SEQ ID NO: 2809. In some embodiments, the AAV particle comprises a viral genome comprising SEQ ID NO: 2174 and a capsid comprising SEQ ID NO: 2871.

In some embodiments, the AAV particle comprises a viral genome comprising SEQ ID NO: 2175 and a VOY101 capsid. In some embodiments, the AAV particle comprises a viral genome comprising SEQ ID NO: 2175 and a VOY201 capsid, in some embodiments, the AAV particle comprises a viral genome comprising SEQ ID NO: 2175 and an AAV9 capsid. In some embodiments, the AAV particle comprises a viral genome comprising SEQ ID NO: 2175 and an AAV9K449R capsid. In some embodiments, the AAV particle comprises a viral genome comprising SEQ ID NO: 2175 and an AAVPHP.B capsid. In some embodiments, the AAV particle comprises a viral genome comprising SEQ ID NO: 2175 and an AAVPHP.N capsid. In some embodiments, the AAV particle comprises a viral genome comprising SEQ ID NO: 2175 and an AAV2 variant capsid. In some embodiments, the AAV particle comprises a viral genome comprising SEQ ID NO: 2175 and an AAV2/3 variant capsid. In some embodiments, the AAV particle comprises a viral genome comprising SEQ ID NO: 2175 and a capsid comprising SEQ ID NO: 1. In some embodiments, the AAV particle comprises a viral genome comprising SEQ ID NO: 2175 and a capsid encoded by a nucleic acid sequence comprising SEQ ID NO: 2. In some embodiments, the AAV particle comprises a viral genome comprising SEQ ID NO: 2175 and a capsid comprising SEQ ID NO: 4534. In some embodiments, the AAV particle comprises a viral genome comprising SEQ ID NO: 2175 and a capsid encoded by a nucleic acid sequence comprising SEQ ID NO: 3. In some embodiments, the AAV particle comprises a viral genome comprising SEQ ID NO: 2175 and a capsid comprising SEQ ID NO: 138. In some embodiments, the AAV particle comprises a viral genome comprising SEQ ID NO: 2175 and a capsid encoded by a nucleic acid sequence comprising SEQ ID NO: 137. In some embodiments, the AAV particle comprises a viral genome comprising SEQ ID NO: 2175 and a capsid comprising SEQ ID NO: 5. In some embodiments, the AAV particle comprises a viral genome comprising SEQ ID NO: 2175 and a capsid encoded by a nucleic acid sequence comprising SEQ ID NO: 6. In some embodiments, the AAV particle comprises a viral genome comprising SEQ ID NO: 2175 and a capsid comprising SEQ ID NO: 4. In some embodiments, the AAV particle comprises a viral genome comprising SEQ ID NO: 2175 and a capsid comprising SEQ ID NO: 11. In some embodiments, the AAV particle comprises a viral genome comprising SEQ ID NO: 2175 and a capsid comprising SEQ ID NO: 2679. In some embodiments, the AAV particle comprises a viral genome comprising SEQ ID NO: 2175 and a capsid comprising SEQ ID NO: 2809. In some embodiments, the AAV particle comprises a viral genome comprising SEQ ID NO: 2175 and a capsid comprising SEQ ID NO: 2871.

In some embodiments, the AAV particle comprises a viral genome comprising SEQ ID NO: 2176 and a VOY101 capsid. In some embodiments, the AAV particle comprises a viral genome comprising SEQ ID NO: 2176 and a VOY201 capsid. In some embodiments, the AAV particle comprises a viral genome comprising SEQ ID NO: 2176 and an AAV9 capsid. In some embodiments, the AAV particle comprises a viral genome comprising SEQ ID NO: 2176 and an AAV9K449R capsid. In some embodiments, the AAV particle comprises a viral genome comprising SEQ ID NO: 2176 and an AAVPHP.B capsid. In some embodiments, the AAV particle comprises a viral genome comprising SEQ ID NO: 2176 and an AAVPHP.N capsid. In some embodiments, the AAV particle comprises a viral genome comprising SEQ ID NO: 2176 and an AAV2 variant capsid. In some embodiments, the AAV particle comprises a viral genome comprising SEQ ID NO: 2176 and an AAV2/3 variant capsid. In some embodiments, the AAV particle comprises a viral genome comprising SEQ ID NO: 2176 and a capsid comprising SEQ ID NO: 1. In some embodiments, the AAV particle comprises a viral genome comprising SEQ ID NO: 2176 and a capsid encoded by a nucleic acid sequence comprising SEQ ID NO: 2. In some embodiments, the AAV particle comprises a viral genome comprising SEQ ID NO: 2176 and a capsid comprising SEQ ID NO: 4534. In some embodiments, the AAV particle comprises a viral genome comprising SEQ ID NO: 2176 and a capsid encoded by a nucleic acid sequence comprising SEQ ID NO: 3. In some embodiments, the AAV particle comprises a viral genome comprising SEQ ID NO: 2176 and a capsid comprising SEQ ID NO: 138. In some embodiments, the AAV particle comprises a viral genome comprising SEQ ID NO: 2176 and a capsid encoded by a nucleic acid sequence comprising SEQ ID NO: 137. In some embodiments, the AAV particle comprises a viral genome comprising SEQ ID NO: 2176 and a capsid comprising SEQ ID NO: 5. In some embodiments, the AAV particle comprises a viral genome comprising SEQ ID NO: 2176 and a capsid encoded by a nucleic acid sequence comprising SEQ ID NO: 6. In some embodiments, the AAV particle comprises a viral genome comprising SEQ ID NO: 2176 and a capsid comprising SEQ ID NO: 4. In some embodiments, the AAV particle comprises a viral genome comprising SEQ ID NO: 2176 and a capsid comprising SEQ ID NO: 11. In some embodiments, the AAV particle comprises a viral genome comprising SEQ ID NO: 2176 and a capsid comprising SEQ ID NO: 2679. In some embodiments, the AAV particle comprises a viral genome comprising SEQ ID NO: 2176 and a capsid comprising SEQ ID NO: 2809. In some embodiments, the AAV particle comprises a viral genome comprising SEQ ID NO: 2176 and a capsid comprising SEQ ID NO: 2871.

In some embodiments, the AAV particle comprises a viral genome comprising SEQ ID NO: 2177 and a VOY101 capsid. In some embodiments, the AAV particle comprises a viral genome comprising SEQ ID NO: 2177 and a VOY201 capsid. In some embodiments, the AAV particle comprises a viral genome comprising SEQ ID NO: 2177 and an AAV9 capsid. In some embodiments, the AAV particle comprises a viral genome comprising SEQ ID NO: 2177 and an AAV9K449R capsid. In some embodiments, the AAV particle comprises a viral genome comprising SEQ ID NO: 2177 and an AAVPHP.B capsid. In some embodiments, the AAV particle comprises a viral genome comprising SEQ ID NO: 2177 and an AAVPHP.N capsid. In some embodiments, the AAV particle comprises a viral genome comprising SEQ ID NO: 2177 and an AAV2 variant capsid. In some embodiments, the AAV particle comprises a viral genome comprising SEQ ID NO: 2177 and an AAV2/3 variant capsid. In some embodiments, the AAV particle comprises a viral genome comprising SEQ ID NO: 2177 and a capsid comprising SEQ ID NO: 1. In some embodiments, the AAV particle comprises a viral genome comprising SEQ ID NO: 2177 and a capsid encoded by a nucleic acid sequence comprising SEQ ID NO: 2. In some embodiments, the AAV particle comprises a viral genome comprising SEQ ID NO: 2177 and a capsid comprising SEQ ID NO: 4534. In some embodiments, the AAV particle comprises a viral genome comprising SEQ ID NO: 2177 and a capsid encoded by a nucleic acid sequence comprising SEQ ID NO: 3. In some embodiments, the AAV particle comprises a viral genome comprising SEQ ID NO: 2177 and a capsid comprising SEQ ID NO: 138. In some embodiments, the AAV particle comprises a viral genome comprising SEQ ID NO: 2177 and a capsid encoded by a nucleic acid sequence comprising SEQ ID NO: 137. In some embodiments, the AAV particle comprises a viral genome comprising SEQ ID NO: 2177 and a capsid comprising SEQ ID NO: 5. In some embodiments, the AAV particle comprises a viral genome comprising SEQ ID NO: 2177 and a capsid encoded by a nucleic acid sequence comprising SEQ ID NO: 6. In some embodiments, the AAV particle comprises a viral genome comprising SEQ ID NO: 2177 and a capsid comprising SEQ ID NO: 4. In some embodiments, the AAV particle comprises a viral genome comprising SEQ ID NO: 2177 and a capsid comprising SEQ ID NO: 11. In some embodiments, the AAV particle comprises a viral genome comprising SEQ ID NO: 2177 and a capsid comprising SEQ ID NO: 2679. In some embodiments, the AAV particle comprises a viral genome comprising SEQ ID NO: 2177 and a capsid comprising SEQ ID NO: 2809. In some embodiments, the AAV particle comprises a viral genome comprising SEQ ID NO: 2177 and a capsid comprising SEQ ID NO: 2871.

In some embodiments, the AAV particle comprises a viral genome comprising SEQ ID NO: 2178 and a VOY101 capsid. In some embodiments, the AAV particle comprises a viral genome comprising SEQ ID NO: 2178 and a VOY201 capsid. In some embodiments, the AAV particle comprises a viral genome comprising SEQ ID NO: 2178 and an AAV9 capsid. In some embodiments, the AAV particle comprises a viral genome comprising SEQ ID NO: 2178 and an AAV9K449R capsid. In some embodiments, the AAV particle comprises a viral genome comprising SEQ ID NO: 2178 and an AAVPHP.B capsid. In some embodiments, the AAV particle comprises a viral genome comprising SEQ ID NO: 2178 and an AAVPHP.N capsid. In some embodiments, the AAV particle comprises a viral genome comprising SEQ ID NO: 2178 and an AAV2 variant capsid. In some embodiments, the AAV particle comprises a viral genome comprising SEQ ID NO: 2178 and an AAV2/3 variant capsid. In some embodiments, the AAV particle comprises a viral genome comprising SEQ ID NO: 2178 and a capsid comprising SEQ ID NO: 1. In some embodiments, the AAV particle comprises a viral genome comprising SEQ ID NO: 2178 and a capsid encoded by a nucleic acid sequence comprising SEQ ID NO: 2. In some embodiments, the AAV particle comprises a viral genome comprising SEQ ID NO: 2178 and a capsid comprising SEQ ID NO: 4534. In some embodiments, the AAV particle comprises a viral genome comprising SEQ ID NO: 2178 and a capsid encoded by a nucleic acid sequence comprising SEQ ID NO: 3. In some embodiments, the AAV particle comprises a viral genome comprising SEQ ID NO: 2178 and a capsid comprising SEQ ID NO: 138. In some embodiments, the AAV particle comprises a viral genome comprising SEQ ID NO: 2178 and a capsid encoded by a nucleic acid sequence comprising SEQ ID NO: 137. In some embodiments, the AAV particle comprises a viral genome comprising SEQ ID NO: 2178 and a capsid comprising SEQ ID NO: 5. In some embodiments, the AAV particle comprises a viral genome comprising SEQ ID NO: 2178 and a capsid encoded by a nucleic acid sequence comprising SEQ ID NO: 6. In some embodiments, the AAV particle comprises a viral genome comprising SEQ ID NO: 2178 and a capsid comprising SEQ ID NO: 4. In some embodiments, the AAV particle comprises a viral genome comprising SEQ ID NO: 2178 and a capsid comprising SEQ ID NO: 11. In some embodiments, the AAV particle comprises a viral genome comprising SEQ ID NO: 2178 and a capsid comprising SEQ ID NO: 2679. In some embodiments, the AAV particle comprises a viral genome comprising SEQ ID NO: 2178 and a capsid comprising SEQ ID NO: 2809. In some embodiments, the AAV particle comprises a viral genome comprising SEQ ID NO: 2178 and a capsid comprising SEQ ID NO: 2871.

In some embodiments, the AAV particle comprises a viral genome comprising SEQ ID NO: 2179 and a VOY101 capsid. In some embodiments, the AAV particle comprises a viral genome comprising SEQ ID NO: 2179 and a VOY201 capsid. In some embodiments, the AAV particle comprises a viral genome comprising SEQ ID NO: 2179 and an AAV9 capsid. In some embodiments, the AAV particle comprises a viral genome comprising SEQ ID NO: 2179 and an AAV9K449R capsid. In some embodiments, the AAV particle comprises a viral genome comprising SEQ ID NO: 2179 and an AAVPHP.B capsid. In some embodiments, the AAV particle comprises a viral genome comprising SEQ ID NO: 2179 and an AAVPHP.N capsid. In some embodiments, the AAV particle comprises a viral genome comprising SEQ ID NO: 2179 and an AAV2 variant capsid. In some embodiments, the AAV particle comprises a viral genome comprising SEQ ID NO: 2179 and an AAV2/3 variant capsid. In some embodiments, the AAV particle comprises a viral genome comprising SEQ ID NO: 2179 and a capsid comprising SEQ ID NO: 1. In some embodiments, the AAV particle comprises a viral genome comprising SEQ ID NO: 2179 and a capsid encoded by a nucleic acid sequence comprising SEQ ID NO: 2. In some embodiments, the AAV particle comprises a viral genome comprising SEQ ID NO: 2179 and a capsid comprising SEQ ID NO: 4534. In some embodiments, the AAV particle comprises a viral genome comprising SEQ ID NO: 2179 and a capsid encoded by a nucleic acid sequence comprising SEQ ID NO: 3. In some embodiments, the AAV particle comprises a viral genome comprising SEQ ID NO: 2179 and a capsid comprising SEQ ID NO: 138. In some embodiments, the AAV particle comprises a viral genome comprising SEQ ID NO: 2179 and a capsid encoded by a nucleic acid sequence comprising SEQ ID NO: 137. In some embodiments, the AAV particle comprises a viral genome comprising SEQ ID NO: 2179 and a capsid comprising SEQ ID NO: 5. In some embodiments, the AAV particle comprises a viral genome comprising SEQ ID NO: 2179 and a capsid encoded by a nucleic acid sequence comprising SEQ ID NO: 6. In some embodiments, the AAV particle comprises a viral genome comprising SEQ ID NO: 2179 and a capsid comprising SEQ ID NO: 4. In some embodiments, the AAV particle comprises a viral genome comprising SEQ ID NO: 2179 and a capsid comprising SEQ ID NO: 11. In some embodiments, the AAV particle comprises a viral genome comprising SEQ ID NO: 2179 and a capsid comprising SEQ ID NO: 2679. In some embodiments, the AAV particle comprises a viral genome comprising SEQ ID NO: 2179 and a capsid comprising SEQ ID NO: 2809. In some embodiments, the AAV particle comprises a viral genome comprising SEQ ID NO: 2179 and a capsid comprising SEQ ID NO: 2871.

In some embodiments, the AAV particle comprises a viral genome comprising SEQ ID NO: 2180 and a VOY101 capsid. In some embodiments, the AAV particle comprises a viral genome comprising SEQ ID NO: 2180 and a VOY201 capsid. In some embodiments, the AAV particle comprises a viral genome comprising SEQ ID NO: 2180 and an AAV9 capsid. In some embodiments, the AAV particle comprises a viral genome comprising SEQ ID NO: 2180 and an AAV9K449R capsid. In some embodiments, the AAV particle comprises a viral genome comprising SEQ ID NO: 2180 and an AAVPHP.B capsid. In some embodiments, the AAV particle comprises a viral genome comprising SEQ ID NO: 2180 and an AAVPHP.N capsid. In some embodiments, the AAV particle comprises a viral genome comprising SEQ ID NO: 2180 and an AAV2 variant capsid. In some embodiments, the AAV particle comprises a viral genome comprising SEQ ID NO: 2180 and an AAV2/3 variant capsid. In some embodiments, the AAV particle comprises a viral genome comprising SEQ ID NO: 2180 and a capsid comprising SEQ ID NO: 1. In some embodiments, the AAV particle comprises a viral genome comprising SEQ ID NO: 2180 and a capsid encoded by a nucleic acid sequence comprising SEQ ID NO: 2. In some embodiments, the AAV particle comprises a viral genome comprising SEQ ID NO: 2180 and a capsid comprising SEQ ID NO: 4534. In some embodiments, the AAV particle comprises a viral genome comprising SEQ ID NO: 2180 and a capsid encoded by a nucleic acid sequence comprising SEQ ID NO: 3. In some embodiments, the AAV particle comprises a viral genome comprising SEQ ID NO: 2180 and a capsid comprising SEQ ID NO: 138. In some embodiments, the AAV particle comprises a viral genome comprising SEQ ID NO: 2180 and a capsid encoded by a nucleic acid sequence comprising SEQ ID NO: 137. In some embodiments, the AAV particle comprises a viral genome comprising SEQ ID NO: 2180 and a capsid comprising SEQ ID NO: 5. In some embodiments, the AAV particle comprises a viral genome comprising SEQ ID NO: 2180 and a capsid encoded by a nucleic acid sequence comprising SEQ ID NO: 6. In some embodiments, the AAV particle comprises a viral genome comprising SEQ ID NO: 2180 and a capsid comprising SEQ ID NO: 4. In some embodiments, the AAV particle comprises a viral genome comprising SEQ ID NO: 2180 and a capsid comprising SEQ ID NO: 11. In some embodiments, the AAV particle comprises a viral genome comprising SEQ ID NO: 2180 and a capsid comprising SEQ ID NO: 2679. In some embodiments, the AAV particle comprises a viral genome comprising SEQ ID NO: 2180 and a capsid comprising SEQ ID NO: 2809. In some embodiments, the AAV particle comprises a viral genome comprising SEQ ID NO: 2180 and a capsid comprising SEQ ID NO: 2871.

In some embodiments, the AAV particle comprises a viral genome comprising SEQ ID NO: 2181 and a VOY101 capsid. In some embodiments, the AAV particle comprises a viral genome comprising SEQ ID NO: 2181 and a VOY201 capsid. In some embodiments, the AAV particle comprises a viral genome comprising SEQ ID NO: 2181 and an AAV9 capsid. In some embodiments, the AAV particle comprises a viral genome comprising SEQ ID NO: 2181 and an AAV9K449R capsid. In some embodiments, the AAV particle comprises a viral genome comprising SEQ ID NO: 2181 and an AAVPHP.B capsid. In some embodiments, the AAV particle comprises a viral genome comprising SEQ ID NO: 2181 and an AAVPHP.N capsid. In some embodiments, the AAV particle comprises a viral genome comprising SEQ ID NO: 2181 and an AAV2 variant capsid. In some embodiments, the AAV particle comprises a viral genome comprising SEQ ID NO: 2181 and an AAV2/3 variant capsid. In some embodiments, the AAV particle comprises a viral genome comprising SEQ ID NO: 2181 and a capsid comprising SEQ ID NO: 1. In some embodiments, the AAV particle comprises a viral genome comprising SEQ ID NO: 2181 and a capsid encoded by a nucleic acid sequence comprising SEQ ID NO: 2. In some embodiments, the AAV particle comprises a viral genome comprising SEQ ID NO: 2181 and a capsid comprising SEQ ID NO: 4534. In some embodiments, the AAV particle comprises a viral genome comprising SEQ ID NO: 2181 and a capsid encoded by a nucleic acid sequence comprising SEQ ID NO: 3. In some embodiments, the AAV particle comprises a viral genome comprising SEQ ID NO: 2181 and a capsid comprising SEQ ID NO: 138. In some embodiments, the AAV particle comprises a viral genome comprising SEQ ID NO: 2181 and a capsid encoded by a nucleic acid sequence comprising SEQ ID NO: 137. In some embodiments, the AAV particle comprises a viral genome comprising SEQ ID NO: 2181 and a capsid comprising SEQ ID NO: 5. In some embodiments, the AAV particle comprises a viral genome comprising SEQ ID NO: 2181 and a capsid encoded by a nucleic acid sequence comprising SEQ ID NO: 6. In some embodiments, the AAV particle comprises a viral genome comprising SEQ ID NO: 2181 and a capsid comprising SEQ ID NO: 4. In some embodiments, the AAV particle comprises a viral genome comprising SEQ ID NO: 2181 and a capsid comprising SEQ ID NO: 11. In some embodiments, the AAV particle comprises a viral genome comprising SEQ ID NO: 2181 and a capsid comprising SEQ ID NO: 2679. In some embodiments, the AAV particle comprises a viral genome comprising SEQ ID NO: 2181 and a capsid comprising SEQ ID NO: 2809. In some embodiments, the AAV particle comprises a viral genome comprising SEQ ID NO: 2181 and a capsid comprising SEQ ID NO: 2871.

In some embodiments, the AAV particle comprises a viral genome comprising SEQ ID NO: 2182 and a VOY101 capsid. In some embodiments, the AAV particle comprises a viral genome comprising SEQ ID NO: 2182 and a VOY201 capsid. In some embodiments, the AAV particle comprises a viral genome comprising SEQ ID NO: 2182 and an AAV9 capsid. In some embodiments, the AAV particle comprises a viral genome comprising SEQ ID NO: 2182 and an AAV9K449R capsid. In some embodiments, the AAV particle comprises a viral genome comprising SEQ ID NO: 2182 and an AAVPHP.B capsid. In some embodiments, the AAV particle comprises a viral genome comprising SEQ ID NO: 2182 and an AAVPHP.N capsid. In some embodiments, the AAV particle comprises a viral genome comprising SEQ ID NO: 2182 and an AAV2 variant capsid. In some embodiments, the AAV particle comprises a viral genome comprising SEQ ID NO: 2182 and an AAV2/3 variant capsid. In some embodiments, the AAV particle comprises a viral genome comprising SEQ ID NO: 2182 and a capsid comprising SEQ ID NO: 1. In some embodiments, the AAV particle comprises a viral genome comprising SEQ ID NO: 2182 and a capsid encoded by a nucleic acid sequence comprising SEQ ID NO: 2. In some embodiments, the AAV particle comprises a viral genome comprising SEQ ID NO: 2182 and a capsid comprising SEQ ID NO: 4534. In some embodiments, the AAV particle comprises a viral genome comprising SEQ ID NO: 2182 and a capsid encoded by a nucleic acid sequence comprising SEQ ID NO: 3. In some embodiments, the AAV particle comprises a viral genome comprising SEQ ID NO: 2182 and a capsid comprising SEQ ID NO: 138. In some embodiments, the AAV particle comprises a viral genome comprising SEQ ID NO: 2182 and a capsid encoded by a nucleic acid sequence comprising SEQ ID NO: 137. In some embodiments, the AAV particle comprises a viral genome comprising SEQ ID NO: 2182 and a capsid comprising SEQ ID NO: 5. In some embodiments, the AAV particle comprises a viral genome comprising SEQ ID NO: 2182 and a capsid encoded by a nucleic acid sequence comprising SEQ ID NO: 6. In some embodiments, the AAV particle comprises a viral genome comprising SEQ ID NO: 2182 and a capsid comprising SEQ ID NO: 4. In some embodiments, the AAV particle comprises a viral genome comprising SEQ ID NO: 2182 and a capsid comprising SEQ ID NO: 11. In some embodiments, the AAV particle comprises a viral genome comprising SEQ ID NO: 2182 and a capsid comprising SEQ ID NO: 2679. In some embodiments, the AAV particle comprises a viral genome comprising SEQ ID NO: 2182 and a capsid comprising SEQ ID NO: 2809. In some embodiments, the AAV particle comprises a viral genome comprising SEQ ID NO: 2182 and a capsid comprising SEQ ID NO: 2871.

In some embodiments, the AAV particle comprises a viral genome comprising SEQ ID NO: 2183 and a VOY101 capsid. In some embodiments, the AAV particle comprises a viral genome comprising SEQ ID NO: 2183 and a VOY201 capsid. In some embodiments, the AAV particle comprises a viral genome comprising SEQ ID NO: 2183 and an AAV9 capsid. In some embodiments, the AAV particle comprises a viral genome comprising SEQ ID NO: 2183 and an AAV9K449R capsid. In some embodiments, the AAV particle comprises a viral genome comprising SEQ ID NO: 2183 and an AAVPHP.B capsid. In some embodiments, the AAV particle comprises a viral genome comprising SEQ ID NO: 2183 and an AAVPHP.N capsid. In some embodiments, the AAV particle comprises a viral genome comprising SEQ ID NO: 2183 and an AAV2 variant capsid. In some embodiments, the AAV particle comprises a viral genome comprising SEQ ID NO: 2183 and an AAV2/3 variant capsid. In some embodiments, the AAV particle comprises a viral genome comprising SEQ ID NO: 2183 and a capsid comprising SEQ ID NO: 1. In some embodiments, the AAV particle comprises a viral genome comprising SEQ ID NO: 2183 and a capsid encoded by a nucleic acid sequence comprising SEQ ID NO: 2. In some embodiments, the AAV particle comprises a viral genome comprising SEQ ID NO: 2183 and a capsid comprising SEQ ID NO: 4534. In some embodiments, the AAV particle comprises a viral genome comprising SEQ ID NO: 2183 and a capsid encoded by a nucleic acid sequence comprising SEQ ID NO: 3. In some embodiments, the AAV particle comprises a viral genome comprising SEQ ID NO: 2183 and a capsid comprising SEQ ID NO: 138. In some embodiments, the AAV particle comprises a viral genome comprising SEQ ID NO: 2183 and a capsid encoded by a nucleic acid sequence comprising SEQ ID NO: 137. In some embodiments, the AAV particle comprises a viral genome comprising SEQ ID NO: 2183 and a capsid comprising SEQ ID NO: 5. In some embodiments, the AAV particle comprises a viral genome comprising SEQ ID NO: 2183 and a capsid encoded by a nucleic acid sequence comprising SEQ ID NO: 6. In some embodiments, the AAV particle comprises a viral genome comprising SEQ ID NO: 2183 and a capsid comprising SEQ ID NO: 4. In some embodiments, the AAV particle comprises a viral genome comprising SEQ ID NO: 2183 and a capsid comprising SEQ ID NO: 11. In some embodiments, the AAV particle comprises a viral genome comprising SEQ ID NO: 2183 and a capsid comprising SEQ ID NO: 2679. In some embodiments, the AAV particle comprises a viral genome comprising SEQ ID NO: 2183 and a capsid comprising SEQ ID NO: 2809. In some embodiments, the AAV particle comprises a viral genome comprising SEQ ID NO: 2183 and a capsid comprising SEQ ID NO: 2871.

In some embodiments, the AAV particle comprises a viral genome comprising SEQ ID NO: 2184 and a VOY101 capsid. In some embodiments, the AAV particle comprises a viral genome comprising SEQ ID NO: 2184 and a VOY201 capsid. In some embodiments, the AAV particle comprises a viral genome comprising SEQ ID NO: 2184 and an AAV9 capsid. In some embodiments, the AAV particle comprises a viral genome comprising SEQ ID NO: 2184 and an AAV9K449R capsid. In some embodiments, the AAV particle comprises a viral genome comprising SEQ ID NO: 2184 and an AAVPHP.B capsid. In some embodiments, the AAV particle comprises a viral genome comprising SEQ ID NO: 2184 and an AAVPHP.N capsid. In some embodiments, the AAV particle comprises a viral genome comprising SEQ ID NO: 2184 and an AAV2 variant capsid. In some embodiments, the AAV particle comprises a viral genome comprising SEQ ID NO: 2184 and an AAV2/3 variant capsid. In some embodiments, the AAV particle comprises a viral genome comprising SEQ ID NO: 2184 and a capsid comprising SEQ ID NO: 1. In some embodiments, the AAV particle comprises a viral genome comprising SEQ ID NO: 2184 and a capsid encoded by a nucleic acid sequence comprising SEQ ID NO: 2. In some embodiments, the AAV particle comprises a viral genome comprising SEQ ID NO: 2184 and a capsid comprising SEQ ID NO: 4534. In some embodiments, the AAV particle comprises a viral genome comprising SEQ ID NO: 2184 and a capsid encoded by a nucleic acid sequence comprising SEQ ID NO: 3. In some embodiments, the AAV particle comprises a viral genome comprising SEQ ID NO: 2184 and a capsid comprising SEQ ID NO: 138. In some embodiments, the AAV particle comprises a viral genome comprising SEQ ID NO: 2184 and a capsid encoded by a nucleic acid sequence comprising SEQ ID NO: 137. In some embodiments, the AAV particle comprises a viral genome comprising SEQ ID NO: 2184 and a capsid comprising SEQ ID NO: 5. In some embodiments, the AAV particle comprises a viral genome comprising SEQ ID NO: 2184 and a capsid encoded by a nucleic acid sequence comprising SEQ ID NO: 6. In some embodiments, the AAV particle comprises a viral genome comprising SEQ ID NO: 2184 and a capsid comprising SEQ ID NO: 4. In some embodiments, the AAV particle comprises a viral genome comprising SEQ ID NO: 2184 and a capsid comprising SEQ ID NO: 11. In some embodiments, the AAV particle comprises a viral genome comprising SEQ ID NO: 2184 and a capsid comprising SEQ ID NO: 2679. In some embodiments, the AAV particle comprises a viral genome comprising SEQ ID NO: 2184 and a capsid comprising SEQ ID NO: 2809. In some embodiments, the AAV particle comprises a viral genome comprising SEQ ID NO: 2184 and a capsid comprising SEQ ID NO: 2871.

In some embodiments, the AAV particle comprises a viral genome comprising SEQ ID NO: 2185 and a VOY101 capsid. In some embodiments, the AAV particle comprises a viral genome comprising SEQ ID NO: 2185 and a VOY201 capsid. In some embodiments, the AAV particle comprises a viral genome comprising SEQ ID NO: 2185 and an AAV9 capsid. In some embodiments, the AAV particle comprises a viral genome comprising SEQ ID NO: 2185 and an AAV9K449R capsid. In some embodiments, the AAV particle comprises a viral genome comprising SEQ ID NO: 2185 and an AAVPHP.B capsid. In some embodiments, the AAV particle comprises a viral genome comprising SEQ ID NO: 2185 and an AAVPHP.N capsid. In some embodiments, the AAV particle comprises a viral genome comprising SEQ ID NO: 2185 and an AAV2 variant capsid. In some embodiments, the AAV particle comprises a viral genome comprising SEQ ID NO: 2185 and an AAV2/3 variant capsid. In some embodiments, the AAV particle comprises a viral genome comprising SEQ ID NO: 2185 and a capsid comprising SEQ ID NO: 1. In some embodiments, the AAV particle comprises a viral genome comprising SEQ ID NO: 2185 and a capsid encoded by a nucleic acid sequence comprising SEQ ID NO: 2. In some embodiments, the AAV particle comprises a viral genome comprising SEQ ID NO: 2185 and a capsid comprising SEQ ID NO: 4534. In some embodiments, the AAV particle comprises a viral genome comprising SEQ ID NO: 2185 and a capsid encoded by a nucleic acid sequence comprising SEQ ID NO: 3. In some embodiments, the AAV particle comprises a viral genome comprising SEQ ID NO: 2185 and a capsid comprising SEQ ID NO: 138. In some embodiments, the AAV particle comprises a viral genome comprising SEQ ID NO: 2185 and a capsid encoded by a nucleic acid sequence comprising SEQ ID NO: 137. In some embodiments, the AAV particle comprises a viral genome comprising SEQ ID NO: 2185 and a capsid comprising SEQ ID NO: 5. In some embodiments, the AAV particle comprises a viral genome comprising SEQ ID NO: 2185 and a capsid encoded by a nucleic acid sequence comprising SEQ ID NO: 6. In some embodiments, the AAV particle comprises a viral genome comprising SEQ ID NO: 2185 and a capsid comprising SEQ ID NO: 4. In some embodiments, the AAV particle comprises a viral genome comprising SEQ ID NO: 2185 and a capsid comprising SEQ ID NO: 11. In some embodiments, the AAV particle comprises a viral genome comprising SEQ ID NO: 2185 and a capsid comprising SEQ ID NO: 2679. In some embodiments, the AAV particle comprises a viral genome comprising SEQ ID NO: 2185 and a capsid comprising SEQ ID NO: 2809. In some embodiments, the AAV particle comprises a viral genome comprising SEQ ID NO: 2185 and a capsid comprising SEQ ID NO: 2871.

In some embodiments, the AAV particle comprises a viral genome comprising SEQ ID NO: 2186 and a VOY101 capsid. In some embodiments, the AAV particle comprises a viral genome comprising SEQ ID NO: 2186 and a VOY201 capsid. In some embodiments, the AAV particle comprises a viral genome comprising SEQ ID NO: 2186 and an AAV9 capsid. In some embodiments, the AAV particle comprises a viral genome comprising SEQ ID NO: 2186 and an AAV9K449R capsid. In some embodiments, the AAV particle comprises a viral genome comprising SEQ ID NO: 2186 and an AAVPHP.B capsid. In some embodiments, the AAV particle comprises a viral genome comprising SEQ ID NO: 2186 and an AAVPHP.N capsid. In some embodiments, the AAV particle comprises a viral genome comprising SEQ ID NO: 2186 and an AAV2 variant capsid. In some embodiments, the AAV particle comprises a viral genome comprising SEQ ID NO: 2186 and an AAV2/3 variant capsid. In some embodiments, the AAV particle comprises a viral genome comprising SEQ ID NO: 2186 and a capsid comprising SEQ ID NO: 1. In some embodiments, the AAV particle comprises a viral genome comprising SEQ ID NO: 2186 and a capsid encoded by a nucleic acid sequence comprising SEQ ID NO: 2. In some embodiments, the AAV particle comprises a viral genome comprising SEQ ID NO: 2186 and a capsid comprising SEQ ID NO: 4534. In some embodiments, the AAV particle comprises a viral genome comprising SEQ ID NO: 2186 and a capsid encoded by a nucleic acid sequence comprising SEQ ID NO: 3. In some embodiments, the AAV particle comprises a viral genome comprising SEQ ID NO: 2186 and a capsid comprising SEQ ID NO: 138. In some embodiments, the AAV particle comprises a viral genome comprising SEQ ID NO: 2186 and a capsid encoded by a nucleic acid sequence comprising SEQ ID NO: 137. In some embodiments, the AAV particle comprises a viral genome comprising SEQ ID NO: 2186 and a capsid comprising SEQ ID NO: 5. In some embodiments, the AAV particle comprises a viral genome comprising SEQ ID NO: 2186 and a capsid encoded by a nucleic acid sequence comprising SEQ ID NO: 6. In some embodiments, the AAV particle comprises a viral genome comprising SEQ ID NO: 2186 and a capsid comprising SEQ ID NO: 4. In some embodiments, the AAV particle comprises a viral genome comprising SEQ ID NO: 2186 and a capsid comprising SEQ ID NO: 11. In some embodiments, the AAV particle comprises a viral genome comprising SEQ ID NO: 2186 and a capsid comprising SEQ ID NO: 2679. In some embodiments, the AAV particle comprises a viral genome comprising SEQ ID NO: 2186 and a capsid comprising SEQ ID NO: 2809. In some embodiments, the AAV particle comprises a viral genome comprising SEQ ID NO: 2186 and a capsid comprising SEQ ID NO: 2871.

In some embodiments, the AAV particle comprises a viral genome comprising SEQ ID NO: 2187 and a VOY101 capsid. In some embodiments, the AAV particle comprises a viral genome comprising SEQ ID NO: 2187 and a VOY201 capsid. In some embodiments, the AAV particle comprises a viral genome comprising SEQ ID NO: 2187 and an AAV9 capsid. In some embodiments, the AAV particle comprises a viral genome comprising SEQ ID NO: 2187 and an AAV9K449R capsid. In some embodiments, the AAV particle comprises a viral genome comprising SEQ ID NO: 2187 and an AAVPHP.B capsid. In some embodiments, the AAV particle comprises a viral genome comprising SEQ ID NO: 2187 and an AAVPHP.N capsid. In some embodiments, the AAV particle comprises a viral genome comprising SEQ ID NO: 2187 and an AAV2 variant capsid. In some embodiments, the AAV particle comprises a viral genome comprising SEQ ID NO: 2187 and an AAV2/3 variant capsid. In some embodiments, the AAV particle comprises a viral genome comprising SEQ ID NO: 2187 and a capsid comprising SEQ ID NO: 1. In some embodiments, the AAV particle comprises a viral genome comprising SEQ ID NO: 2187 and a capsid encoded by a nucleic acid sequence comprising SEQ ID NO: 2. In some embodiments, the AAV particle comprises a viral genome comprising SEQ ID NO: 2187 and a capsid comprising SEQ ID NO: 4534. In some embodiments, the AAV particle comprises a viral genome comprising SEQ ID NO: 2187 and a capsid encoded by a nucleic acid sequence comprising SEQ ID NO: 3. In some embodiments, the AAV particle comprises a viral genome comprising SEQ ID NO: 2187 and a capsid comprising SEQ ID NO: 138. In some embodiments, the AAV particle comprises a viral genome comprising SEQ ID NO: 2187 and a capsid encoded by a nucleic acid sequence comprising SEQ ID NO: 137. In some embodiments, the AAV particle comprises a viral genome comprising SEQ ID NO: 2187 and a capsid comprising SEQ ID NO: 5. In some embodiments, the AAV particle comprises a viral genome comprising SEQ ID NO: 2187 and a capsid encoded by a nucleic acid sequence comprising SEQ ID NO: 6. In some embodiments, the AAV particle comprises a viral genome comprising SEQ ID NO: 2187 and a capsid comprising SEQ ID NO: 4. In some embodiments, the AAV particle comprises a viral genome comprising SEQ ID NO: 2187 and a capsid comprising SEQ ID NO: 11. In some embodiments, the AAV particle comprises a viral genome comprising SEQ ID NO: 2187 and a capsid comprising SEQ ID NO: 2679. In some embodiments, the AAV particle comprises a viral genome comprising SEQ ID NO: 2187 and a capsid comprising SEQ ID NO: 2809. In some embodiments, the AAV particle comprises a viral genome comprising SEQ ID NO: 2187 and a capsid comprising SEQ ID NO: 2871.

In some embodiments, the AAV particle comprises a viral genome comprising SEQ ID NO: 2188 and a VOY101 capsid. In some embodiments, the AAV particle comprises a viral genome comprising SEQ ID NO: 2188 and a VOY201 capsid. In some embodiments, the AAV particle comprises a viral genome comprising SEQ ID NO: 2188 and an AAV9 capsid. In some embodiments, the AAV particle comprises a viral genome comprising SEQ ID NO: 2188 and an AAV9K449R capsid. In some embodiments, the AAV particle comprises a viral genome comprising SEQ ID NO: 2188 and an AAVPHP.B capsid. In some embodiments, the AAV particle comprises a viral genome comprising SEQ ID NO: 2188 and an AAVPHP.N capsid. In some embodiments, the AAV particle comprises a viral genome comprising SEQ ID NO: 2188 and an AAV2 variant capsid. In some embodiments, the AAV particle comprises a viral genome comprising SEQ ID NO: 2188 and an AAV2/3 variant capsid. In some embodiments, the AAV particle comprises a viral genome comprising SEQ ID NO: 2188 and a capsid comprising SEQ ID NO: 1. In some embodiments, the AAV particle comprises a viral genome comprising SEQ ID NO: 2188 and a capsid encoded by a nucleic acid sequence comprising SEQ ID NO: 2. In some embodiments, the AAV particle comprises a viral genome comprising SEQ ID NO: 2188 and a capsid comprising SEQ ID NO: 4534. In some embodiments, the AAV particle comprises a viral genome comprising SEQ ID NO: 2188 and a capsid encoded by a nucleic acid sequence comprising SEQ ID NO: 3. In some embodiments, the AAV particle comprises a viral genome comprising SEQ ID NO: 2188 and a capsid comprising SEQ ID NO: 138. In some embodiments, the AAV particle comprises a viral genome comprising SEQ ID NO: 2188 and a capsid encoded by a nucleic acid sequence comprising SEQ ID NO: 137. In some embodiments, the AAV particle comprises a viral genome comprising SEQ ID NO: 2188 and a capsid comprising SEQ ID NO: 5. In some embodiments, the AAV particle comprises a viral genome comprising SEQ ID NO: 2188 and a capsid encoded by a nucleic acid sequence comprising SEQ ID NO: 6. In some embodiments, the AAV particle comprises a viral genome comprising SEQ ID NO: 2188 and a capsid comprising SEQ ID NO: 4. In some embodiments, the AAV particle comprises a viral genome comprising SEQ ID NO: 2188 and a capsid comprising SEQ ID NO: 11. In some embodiments, the AAV particle comprises a viral genome comprising SEQ ID NO: 2188 and a capsid comprising SEQ ID NO: 2679. In some embodiments, the AAV particle comprises a viral genome comprising SEQ ID NO: 2188 and a capsid comprising SEQ ID NO: 2809. In some embodiments, the AAV particle comprises a viral genome comprising SEQ ID NO: 2188 and a capsid comprising SEQ ID NO: 2871.

In some embodiments, the AAV particle comprises a viral genome comprising SEQ ID NO: 2189 and a VOY101 capsid. In some embodiments, the AAV particle comprises a viral genome comprising SEQ ID NO: 2189 and a VOY201 capsid. In some embodiments, the AAV particle comprises a viral genome comprising SEQ ID NO: 2189 and an AAV9 capsid. In some embodiments, the AAV particle comprises a viral genome comprising SEQ ID NO: 2189 and an AAV9K449R capsid. In some embodiments, the AAV particle comprises a viral genome comprising SEQ ID NO: 2189 and an AAVPHP.B capsid. In some embodiments, the AAV particle comprises a viral genome comprising SEQ ID NO: 2189 and an AAVPHP.N capsid. In some embodiments, the AAV particle comprises a viral genome comprising SEQ ID NO: 2189 and an AAV2 variant capsid. In some embodiments, the AAV particle comprises a viral genome comprising SEQ ID NO: 2189 and an AAV2/3 variant capsid. In some embodiments, the AAV particle comprises a viral genome comprising SEQ ID NO: 2189 and a capsid comprising SEQ ID NO: 1. In some embodiments, the AAV particle comprises a viral genome comprising SEQ ID NO: 2189 and a capsid encoded by a nucleic acid sequence comprising SEQ ID NO: 2. In some embodiments, the AAV particle comprises a viral genome comprising SEQ ID NO: 2189 and a capsid comprising SEQ ID NO: 4534. In some embodiments, the AAV particle comprises a viral genome comprising SEQ ID NO: 2189 and a capsid encoded by a nucleic acid sequence comprising SEQ ID NO: 3. In some embodiments, the AAV particle comprises a viral genome comprising SEQ ID NO: 2189 and a capsid comprising SEQ ID NO: 138. In some embodiments, the AAV particle comprises a viral genome comprising SEQ ID NO: 2189 and a capsid encoded by a nucleic acid sequence comprising SEQ ID NO: 137. In some embodiments, the AAV particle comprises a viral genome comprising SEQ ID NO: 2189 and a capsid comprising SEQ ID NO: 5. In some embodiments, the AAV particle comprises a viral genome comprising SEQ ID NO: 2189 and a capsid encoded by a nucleic acid sequence comprising SEQ ID NO: 6. In some embodiments, the AAV particle comprises a viral genome comprising SEQ ID NO: 2189 and a capsid comprising SEQ ID NO: 4. In some embodiments, the AAV particle comprises a viral genome comprising SEQ ID NO: 2189 and a capsid comprising SEQ ID NO: 11. In some embodiments, the AAV particle comprises a viral genome comprising SEQ ID NO: 2189 and a capsid comprising SEQ ID NO: 2679. In some embodiments, the AAV particle comprises a viral genome comprising SEQ ID NO: 2189 and a capsid comprising SEQ ID NO: 2809. In some embodiments, the AAV particle comprises a viral genome comprising SEQ ID NO: 2189 and a capsid comprising SEQ ID NO: 2871.

In some embodiments, the AAV particle comprises a viral genome comprising SEQ ID NO: 2190 and a VOY101 capsid. In some embodiments, the AAV particle comprises a viral genome comprising SEQ ID NO: 2190 and a VOY201 capsid. In some embodiments, the AAV particle comprises a viral genome comprising SEQ ID NO: 2190 and an AAV9 capsid. In some embodiments, the AAV particle comprises a viral genome comprising SEQ ID NO: 2190 and an AAV9K449R capsid. In some embodiments, the AAV particle comprises a viral genome comprising SEQ ID NO: 2190 and an AAVPHP.B capsid. In some embodiments, the AAV particle comprises a viral genome comprising SEQ ID NO: 2190 and an AAVPHP.N capsid. In some embodiments, the AAV particle comprises a viral genome comprising SEQ ID NO: 2190 and an AAV2 variant capsid. In some embodiments, the AAV particle comprises a viral genome comprising SEQ ID NO: 2190 and an AAV2/3 variant capsid. In some embodiments, the AAV particle comprises a viral genome comprising SEQ ID NO: 2190 and a capsid comprising SEQ ID NO: 1. In some embodiments, the AAV particle comprises a viral genome comprising SEQ ID NO: 2190 and a capsid encoded by a nucleic acid sequence comprising SEQ ID NO: 2. In some embodiments, the AAV particle comprises a viral genome comprising SEQ ID NO: 2190 and a capsid comprising SEQ ID NO: 4534. In some embodiments, the AAV particle comprises a viral genome comprising SEQ ID NO: 2190 and a capsid encoded by a nucleic acid sequence comprising SEQ ID NO: 3. In some embodiments, the AAV particle comprises a viral genome comprising SEQ ID NO: 2190 and a capsid comprising SEQ ID NO: 138. In some embodiments, the AAV particle comprises a viral genome comprising SEQ ID NO: 2190 and a capsid encoded by a nucleic acid sequence comprising SEQ ID NO: 137. In some embodiments, the AAV particle comprises a viral genome comprising SEQ ID NO: 2190 and a capsid comprising SEQ ID NO: 5. In some embodiments, the AAV particle comprises a viral genome comprising SEQ ID NO: 2190 and a capsid encoded by a nucleic acid sequence comprising SEQ ID NO: 6. In some embodiments, the AAV particle comprises a viral genome comprising SEQ ID NO: 2190 and a capsid comprising SEQ ID NO: 4. In some embodiments, the AAV particle comprises a viral genome comprising SEQ ID NO: 2190 and a capsid comprising SEQ ID NO: 11. In some embodiments, the AAV particle comprises a viral genome comprising SEQ ID NO: 2190 and a capsid comprising SEQ ID NO: 2679. In some embodiments, the AAV particle comprises a viral genome comprising SEQ ID NO: 2190 and a capsid comprising SEQ ID NO: 2809. In some embodiments, the AAV particle comprises a viral genome comprising SEQ ID NO: 2190 and a capsid comprising SEQ ID NO: 2871.

In some embodiments, the AAV particle comprises a viral genome comprising SEQ ID NO: 2191 and a VOY101 capsid. In some embodiments, the AAV particle comprises a viral genome comprising SEQ ID NO: 2191 and a VOY201 capsid. In some embodiments, the AAV particle comprises a viral genome comprising SEQ ID NO: 2191 and an AAV9 capsid. In some embodiments, the AAV particle comprises a viral genome comprising SEQ ID NO: 2191 and an AAV9K449R capsid. In some embodiments, the AAV particle comprises a viral genome comprising SEQ ID NO: 2191 and an AAVPHP.B capsid. In some embodiments, the AAV particle comprises a viral genome comprising SEQ ID NO: 2191 and an AAVPHP.N capsid. In some embodiments, the AAV particle comprises a viral genome comprising SEQ ID NO: 2191 and an AAV2 variant capsid. In some embodiments, the AAV particle comprises a viral genome comprising SEQ ID NO: 2191 and an AAV2/3 variant capsid. In some embodiments, the AAV particle comprises a viral genome comprising SEQ ID NO: 2191 and a capsid comprising SEQ ID NO: 1. In some embodiments, the AAV particle comprises a viral genome comprising SEQ ID NO: 2191 and a capsid encoded by a nucleic acid sequence comprising SEQ ID NO: 2. In some embodiments, the AAV particle comprises a viral genome comprising SEQ ID NO: 2191 and a capsid comprising SEQ ID NO: 4534. In some embodiments, the AAV particle comprises a viral genome comprising SEQ ID NO: 2191 and a capsid encoded by a nucleic acid sequence comprising SEQ ID NO: 3. In some embodiments, the AAV particle comprises a viral genome comprising SEQ ID NO: 2191 and a capsid comprising SEQ ID NO: 138. In some embodiments, the AAV particle comprises a viral genome comprising SEQ ID NO: 2191 and a capsid encoded by a nucleic acid sequence comprising SEQ ID NO: 137. In some embodiments, the AAV particle comprises a viral genome comprising SEQ ID NO: 2191 and a capsid comprising SEQ ID NO: 5. In some embodiments, the AAV particle comprises a viral genome comprising SEQ ID NO: 2191 and a capsid encoded by a nucleic acid sequence comprising SEQ ID NO: 6. In some embodiments, the AAV particle comprises a viral genome comprising SEQ ID NO: 2191 and a capsid comprising SEQ ID NO: 4. In some embodiments, the AAV particle comprises a viral genome comprising SEQ ID NO: 2191 and a capsid comprising SEQ ID NO: 11. In some embodiments, the AAV particle comprises a viral genome comprising SEQ ID NO: 2191 and a capsid comprising SEQ ID NO: 2679. In some embodiments, the AAV particle comprises a viral genome comprising SEQ ID NO: 2191 and a capsid comprising SEQ ID NO: 2809. In some embodiments, the AAV particle comprises a viral genome comprising SEQ ID NO: 2191 and a capsid comprising SEQ ID NO: 2871.

In some embodiments, the AAV particle comprises a viral genome comprising SEQ ID NO: 2192 and a VOY101 capsid. In some embodiments, the AAV particle comprises a viral genome comprising SEQ ID NO: 2192 and a VOY201 capsid. In some embodiments, the AAV particle comprises a viral genome comprising SEQ ID NO: 2192 and an AAV9 capsid. In some embodiments, the AAV particle comprises a viral genome comprising SEQ ID NO: 2192 and an AAV9K449R capsid. In some embodiments, the AAV particle comprises a viral genome comprising SEQ ID NO: 2192 and an AAVPHP.B capsid. In some embodiments, the AAV particle comprises a viral genome comprising SEQ ID NO: 2192 and an AAVPHP.N capsid. In some embodiments, the AAV particle comprises a viral genome comprising SEQ ID NO: 2192 and an AAV2 variant capsid. In some embodiments, the AAV particle comprises a viral genome comprising SEQ ID NO: 2192 and an AAV2/3 variant capsid. In some embodiments, the AAV particle comprises a viral genome comprising SEQ ID NO: 2192 and a capsid comprising SEQ ID NO: 1. In some embodiments, the AAV particle comprises a viral genome comprising SEQ ID NO: 2192 and a capsid encoded by a nucleic acid sequence comprising SEQ ID NO: 2. In some embodiments, the AAV particle comprises a viral genome comprising SEQ ID NO: 2192 and a capsid comprising SEQ ID NO: 4534. In some embodiments, the AAV particle comprises a viral genome comprising SEQ ID NO: 2192 and a capsid encoded by a nucleic acid sequence comprising SEQ ID NO: 3. In some embodiments, the AAV particle comprises a viral genome comprising SEQ ID NO: 2192 and a capsid comprising SEQ ID NO: 138. In some embodiments, the AAV particle comprises a viral genome comprising SEQ ID NO: 2192 and a capsid encoded by a nucleic acid sequence comprising SEQ ID NO: 137. In some embodiments, the AAV particle comprises a viral genome comprising SEQ ID NO: 2192 and a capsid comprising SEQ ID NO: 5. In some embodiments, the AAV particle comprises a viral genome comprising SEQ ID NO: 2192 and a capsid encoded by a nucleic acid sequence comprising SEQ ID NO: 6. In some embodiments, the AAV particle comprises a viral genome comprising SEQ ID NO: 2192 and a capsid comprising SEQ ID NO: 4. In some embodiments, the AAV particle comprises a viral genome comprising SEQ ID NO: 2192 and a capsid comprising SEQ ID NO: 11. In some embodiments, the AAV particle comprises a viral genome comprising SEQ ID NO: 2192 and a capsid comprising SEQ ID NO: 2679. In some embodiments, the AAV particle comprises a viral genome comprising SEQ ID NO: 2192 and a capsid comprising SEQ ID NO: 2809. In some embodiments, the AAV particle comprises a viral genome comprising SEQ ID NO: 2192 and a capsid comprising SEQ ID NO: 2871.

In some embodiments, the AAV particle comprises a viral genome comprising SEQ ID NO: 2193 and a VOY101 capsid. In some embodiments, the AAV particle comprises a viral genome comprising SEQ ID NO: 2193 and a VOY201 capsid. In some embodiments, the AAV particle comprises a viral genome comprising SEQ ID NO: 2193 and an AAV9 capsid. In some embodiments, the AAV particle comprises a viral genome comprising SEQ ID NO: 2193 and an AAV9K449R capsid. In some embodiments, the AAV particle comprises a viral genome comprising SEQ ID NO: 2193 and an AAVPHP.B capsid. In some embodiments, the AAV particle comprises a viral genome comprising SEQ ID NO: 2193 and an AAVPHP.N capsid. In some embodiments, the AAV particle comprises a viral genome comprising SEQ ID NO: 2193 and an AAV2 variant capsid. In some embodiments, the AAV particle comprises a viral genome comprising SEQ ID NO: 2193 and an AAV2/3 variant capsid. In some embodiments, the AAV particle comprises a viral genome comprising SEQ ID NO: 2193 and a capsid comprising SEQ ID NO: 1. In some embodiments, the AAV particle comprises a viral genome comprising SEQ ID NO: 2193 and a capsid encoded by a nucleic acid sequence comprising SEQ ID NO: 2. In some embodiments, the AAV particle comprises a viral genome comprising SEQ ID NO: 2193 and a capsid comprising SEQ ID NO: 4534. In some embodiments, the AAV particle comprises a viral genome comprising SEQ ID NO: 2193 and a capsid encoded by a nucleic acid sequence comprising SEQ ID NO: 3. In some embodiments, the AAV particle comprises a viral genome comprising SEQ ID NO: 2193 and a capsid comprising SEQ ID NO: 138. In some embodiments, the AAV particle comprises a viral genome comprising SEQ ID NO: 2193 and a capsid encoded by a nucleic acid sequence comprising SEQ ID NO: 137. In some embodiments, the AAV particle comprises a viral genome comprising SEQ ID NO: 2193 and a capsid comprising SEQ ID NO: 5. In some embodiments, the AAV particle comprises a viral genome comprising SEQ ID NO: 2193 and a capsid encoded by a nucleic acid sequence comprising SEQ ID NO: 6. In some embodiments, the AAV particle comprises a viral genome comprising SEQ ID NO: 2193 and a capsid comprising SEQ ID NO: 4. In some embodiments, the AAV particle comprises a viral genome comprising SEQ ID NO: 2193 and a capsid comprising SEQ ID NO: 11. In some embodiments, the AAV particle comprises a viral genome comprising SEQ ID NO: 2193 and a capsid comprising SEQ ID NO: 2679. In some embodiments, the AAV particle comprises a viral genome comprising SEQ ID NO: 2193 and a capsid comprising SEQ ID NO: 2809. In some embodiments, the AAV particle comprises a viral genome comprising SEQ ID NO: 2193 and a capsid comprising SEQ ID NO: 2871.

In some embodiments, the AAV particle comprises a viral genome comprising SEQ ID NO: 2194 and a VOY101 capsid. In some embodiments, the AAV particle comprises a viral genome comprising SEQ ID NO: 2194 and a VOY201 capsid. In some embodiments, the AAV particle comprises a viral genome comprising SEQ ID NO: 2194 and an AAV9 capsid. In some embodiments, the AAV particle comprises a viral genome comprising SEQ ID NO: 2194 and an AAV9K449R capsid. In some embodiments, the AAV particle comprises a viral genome comprising SEQ ID NO: 2194 and an AAVPHP.B capsid. In some embodiments, the AAV particle comprises a viral genome comprising SEQ ID NO: 2194 and an AAVPHP.N capsid. In some embodiments, the AAV particle comprises a viral genome comprising SEQ ID NO: 2194 and an AAV2 variant capsid. In some embodiments, the AAV particle comprises a viral genome comprising SEQ ID NO: 2194 and an AAV2/3 variant capsid. In some embodiments, the AAV particle comprises a viral genome comprising SEQ ID NO: 2194 and a capsid comprising SEQ ID NO: 1. In some embodiments, the AAV particle comprises a viral genome comprising SEQ ID NO: 2194 and a capsid encoded by a nucleic acid sequence comprising SEQ ID NO: 2. In some embodiments, the AAV particle comprises a viral genome comprising SEQ ID NO: 2194 and a capsid comprising SEQ ID NO: 4534. In some embodiments, the AAV particle comprises a viral genome comprising SEQ ID NO: 2194 and a capsid encoded by a nucleic acid sequence comprising SEQ ID NO: 3. In some embodiments, the AAV particle comprises a viral genome comprising SEQ ID NO: 2194 and a capsid comprising SEQ ID NO: 138. In some embodiments, the AAV particle comprises a viral genome comprising SEQ ID NO: 2194 and a capsid encoded by a nucleic acid sequence comprising SEQ ID NO: 137. In some embodiments, the AAV particle comprises a viral genome comprising SEQ ID NO: 2194 and a capsid comprising SEQ ID NO: 5. In some embodiments, the AAV particle comprises a viral genome comprising SEQ ID NO: 2194 and a capsid encoded by a nucleic acid sequence comprising SEQ ID NO: 6. In some embodiments, the AAV particle comprises a viral genome comprising SEQ ID NO: 2194 and a capsid comprising SEQ ID NO: 4. In some embodiments, the AAV particle comprises a viral genome comprising SEQ ID NO: 2194 and a capsid comprising SEQ ID NO: 11. In some embodiments, the AAV particle comprises a viral genome comprising SEQ ID NO: 2194 and a capsid comprising SEQ ID NO: 2679. In some embodiments, the AAV particle comprises a viral genome comprising SEQ ID NO: 2194 and a capsid comprising SEQ ID NO: 2809. In some embodiments, the AAV particle comprises a viral genome comprising SEQ ID NO: 2194 and a capsid comprising SEQ ID NO: 2871.

In some embodiments, the AAV particle comprises a viral genome comprising SEQ ID NO: 2195 and a VOY101 capsid. In some embodiments, the AAV particle comprises a viral genome comprising SEQ ID NO: 2195 and a VOY201 capsid. In some embodiments, the AAV particle comprises a viral genome comprising SEQ ID NO: 2195 and an AAV9 capsid. In some embodiments, the AAV particle comprises a viral genome comprising SEQ ID NO: 2195 and an AAV9K449R capsid. In some embodiments, the AAV particle comprises a viral genome comprising SEQ ID NO: 2195 and an AAVPHP.B capsid. In some embodiments, the AAV particle comprises a viral genome comprising SEQ ID NO: 2195 and an AAVPHP.N capsid. In some embodiments, the AAV particle comprises a viral genome comprising SEQ ID NO: 2195 and an AAV2 variant capsid. In some embodiments, the AAV particle comprises a viral genome comprising SEQ ID NO: 2195 and an AAV2/3 variant capsid. In some embodiments, the AAV particle comprises a viral genome comprising SEQ ID NO: 2195 and a capsid comprising SEQ ID NO: 1. In some embodiments, the AAV particle comprises a viral genome comprising SEQ ID NO: 2195 and a capsid encoded by a nucleic acid sequence comprising SEQ ID NO: 2. In some embodiments, the AAV particle comprises a viral genome comprising SEQ ID NO: 2195 and a capsid comprising SEQ ID NO: 4534. In some embodiments, the AAV particle comprises a viral genome comprising SEQ ID NO: 2195 and a capsid encoded by a nucleic acid sequence comprising SEQ ID NO: 3. In some embodiments, the AAV particle comprises a viral genome comprising SEQ ID NO: 2195 and a capsid comprising SEQ ID NO: 138. In some embodiments, the AAV particle comprises a viral genome comprising SEQ ID NO: 2195 and a capsid encoded by a nucleic acid sequence comprising SEQ ID NO: 137. In some embodiments, the AAV particle comprises a viral genome comprising SEQ ID NO: 2195 and a capsid comprising SEQ ID NO: 5. In some embodiments, the AAV particle comprises a viral genome comprising SEQ ID NO: 2195 and a capsid encoded by a nucleic acid sequence comprising SEQ ID NO: 6. In some embodiments, the AAV particle comprises a viral genome comprising SEQ ID NO: 2195 and a capsid comprising SEQ ID NO: 4. In some embodiments, the AAV particle comprises a viral genome comprising SEQ ID NO: 2195 and a capsid comprising SEQ ID NO: 11. In some embodiments, the AAV particle comprises a viral genome comprising SEQ ID NO: 2195 and a capsid comprising SEQ ID NO: 2679. In some embodiments, the AAV particle comprises a viral genome comprising SEQ ID NO: 2195 and a capsid comprising SEQ ID NO: 2809. In some embodiments, the AAV particle comprises a viral genome comprising SEQ ID NO: 2195 and a capsid comprising SEQ ID NO: 2871.

In some embodiments, the AAV particle comprises a viral genome comprising SEQ ID NO: 2196 and a VOY101 capsid. In some embodiments, the AAV particle comprises a viral genome comprising SEQ ID NO: 2196 and a VOY201 capsid. In some embodiments, the AAV particle comprises a viral genome comprising SEQ ID NO: 2196 and an AAV9 capsid. In some embodiments, the AAV particle comprises a viral genome comprising SEQ ID NO: 2196 and an AAV9K449R capsid. In some embodiments, the AAV particle comprises a viral genome comprising SEQ ID NO: 2196 and an AAVPHP.B capsid. In some embodiments, the AAV particle comprises a viral genome comprising SEQ ID NO: 2196 and an AAVPHP.N capsid. In some embodiments, the AAV particle comprises a viral genome comprising SEQ ID NO: 2196 and an AAV2 variant capsid. In some embodiments, the AAV particle comprises a viral genome comprising SEQ ID NO: 2196 and an AAV2/3 variant capsid. In some embodiments, the AAV particle comprises a viral genome comprising SEQ ID NO: 2196 and a capsid comprising SEQ ID NO: 1. In some embodiments, the AAV particle comprises a viral genome comprising SEQ ID NO: 2196 and a capsid encoded by a nucleic acid sequence comprising SEQ ID NO: 2. In some embodiments, the AAV particle comprises a viral genome comprising SEQ ID NO: 2196 and a capsid comprising SEQ ID NO: 4534. In some embodiments, the AAV particle comprises a viral genome comprising SEQ ID NO: 2196 and a capsid encoded by a nucleic acid sequence comprising SEQ ID NO: 3. In some embodiments, the AAV particle comprises a viral genome comprising SEQ ID NO: 2196 and a capsid comprising SEQ ID NO: 138. In some embodiments, the AAV particle comprises a viral genome comprising SEQ ID NO: 2196 and a capsid encoded by a nucleic acid sequence comprising SEQ ID NO: 137. In some embodiments, the AAV particle comprises a viral genome comprising SEQ ID NO: 2196 and a capsid comprising SEQ ID NO: 5. In some embodiments, the AAV particle comprises a viral genome comprising SEQ ID NO: 2196 and a capsid encoded by a nucleic acid sequence comprising SEQ ID NO: 6. In some embodiments, the AAV particle comprises a viral genome comprising SEQ ID NO: 2196 and a capsid comprising SEQ ID NO: 4. In some embodiments, the AAV particle comprises a viral genome comprising SEQ ID NO: 2196 and a capsid comprising SEQ ID NO: 11. In some embodiments, the AAV particle comprises a viral genome comprising SEQ ID NO: 2196 and a capsid comprising SEQ ID NO: 2679. In some embodiments, the AAV particle comprises a viral genome comprising SEQ ID NO: 2196 and a capsid comprising SEQ ID NO: 2809. In some embodiments, the AAV particle comprises a viral genome comprising SEQ ID NO: 2196 and a capsid comprising SEQ ID NO: 2871.

In some embodiments, the AAV particle comprises a viral genome comprising SEQ ID NO: 2197 and a VOY101 capsid. In some embodiments, the AAV particle comprises a viral genome comprising SEQ ID NO: 2197 and a VOY201 capsid. In some embodiments, the AAV particle comprises a viral genome comprising SEQ ID NO: 2197 and an AAV9 capsid. In some embodiments, the AAV particle comprises a viral genome comprising SEQ ID NO: 2197 and an AAV9K449R capsid. In some embodiments, the AAV particle comprises a viral genome comprising SEQ ID NO: 2197 and an AAVPHP.B capsid. In some embodiments, the AAV particle comprises a viral genome comprising SEQ ID NO: 2197 and an AAVPHP.N capsid. In some embodiments, the AAV particle comprises a viral genome comprising SEQ ID NO: 2197 and an AAV2 variant capsid. In some embodiments, the AAV particle comprises a viral genome comprising SEQ ID NO: 2197 and an AAV2/3 variant capsid. In some embodiments, the AAV particle comprises a viral genome comprising SEQ ID NO: 2197 and a capsid comprising SEQ ID NO: 1. In some embodiments, the AAV particle comprises a viral genome comprising SEQ ID NO: 2197 and a capsid encoded by a nucleic acid sequence comprising SEQ ID NO: 2. In some embodiments, the AAV particle comprises a viral genome comprising SEQ ID NO: 2197 and a capsid comprising SEQ ID NO: 4534. In some embodiments, the AAV particle comprises a viral genome comprising SEQ ID NO: 2197 and a capsid encoded by a nucleic acid sequence comprising SEQ ID NO: 3. In some embodiments, the AAV particle comprises a viral genome comprising SEQ ID NO: 2197 and a capsid comprising SEQ ID NO: 138. In some embodiments, the AAV particle comprises a viral genome comprising SEQ ID NO: 2197 and a capsid encoded by a nucleic acid sequence comprising SEQ ID NO: 137. In some embodiments, the AAV particle comprises a viral genome comprising SEQ ID NO: 2197 and a capsid comprising SEQ ID NO: 5. In some embodiments, the AAV particle comprises a viral genome comprising SEQ ID NO: 2197 and a capsid encoded by a nucleic acid sequence comprising SEQ ID NO: 6. In some embodiments, the AAV particle comprises a viral genome comprising SEQ ID NO: 2197 and a capsid comprising SEQ ID NO: 4. In some embodiments, the AAV particle comprises a viral genome comprising SEQ ID NO: 2197 and a capsid comprising SEQ ID NO: 11. In some embodiments, the AAV particle comprises a viral genome comprising SEQ ID NO: 2197 and a capsid comprising SEQ ID NO: 2679. In some embodiments, the AAV particle comprises a viral genome comprising SEQ ID NO: 2197 and a capsid comprising SEQ ID NO: 2809. In some embodiments, the AAV particle comprises a viral genome comprising SEQ ID NO: 2197 and a capsid comprising SEQ ID NO: 2871.

In some embodiments, the AAV particle comprises a viral genome comprising SEQ ID NO: 2198 and a VOY101 capsid. In some embodiments, the AAV particle comprises a viral genome comprising SEQ ID NO: 2198 and a VOY201 capsid. In some embodiments, the AAV particle comprises a viral genome comprising SEQ ID NO: 2198 and an AAV9 capsid. In some embodiments, the AAV particle comprises a viral genome comprising SEQ ID NO: 2198 and an AAV9K449R capsid. In some embodiments, the AAV particle comprises a viral genome comprising SEQ ID NO: 2198 and an AAVPHP.B capsid. In some embodiments, the AAV particle comprises a viral genome comprising SEQ ID NO: 2198 and an AAVPHP.N capsid. In some embodiments, the AAV particle comprises a viral genome comprising SEQ ID NO: 2198 and an AAV2 variant capsid. In some embodiments, the AAV particle comprises a viral genome comprising SEQ ID NO: 2198 and an AAV2/3 variant capsid. In some embodiments, the AAV particle comprises a viral genome comprising SEQ ID NO: 2198 and a capsid comprising SEQ ID NO: 1. In some embodiments, the AAV particle comprises a viral genome comprising SEQ ID NO: 2198 and a capsid encoded by a nucleic acid sequence comprising SEQ ID NO: 2. In some embodiments, the AAV particle comprises a viral genome comprising SEQ ID NO: 2198 and a capsid comprising SEQ ID NO: 4534. In some embodiments, the AAV particle comprises a viral genome comprising SEQ ID NO: 2198 and a capsid encoded by a nucleic acid sequence comprising SEQ ID NO: 3. In some embodiments, the AAV particle comprises a viral genome comprising SEQ ID NO: 2198 and a capsid comprising SEQ ID NO: 138. In some embodiments, the AAV particle comprises a viral genome comprising SEQ ID NO: 2198 and a capsid encoded by a nucleic acid sequence comprising SEQ ID NO: 137. In some embodiments, the AAV particle comprises a viral genome comprising SEQ ID NO: 2198 and a capsid comprising SEQ ID NO: 5. In some embodiments, the AAV particle comprises a viral genome comprising SEQ ID NO: 2198 and a capsid encoded by a nucleic acid sequence comprising SEQ ID NO: 6. In some embodiments, the AAV particle comprises a viral genome comprising SEQ ID NO: 2198 and a capsid comprising SEQ ID NO: 4. In some embodiments, the AAV particle comprises a viral genome comprising SEQ ID NO: 2198 and a capsid comprising SEQ ID NO: 11. In some embodiments, the AAV particle comprises a viral genome comprising SEQ ID NO: 2198 and a capsid comprising SEQ ID NO: 2679. In some embodiments, the AAV particle comprises a viral genome comprising SEQ ID NO: 2198 and a capsid comprising SEQ ID NO: 2809. In some embodiments, the AAV particle comprises a viral genome comprising SEQ ID NO: 2198 and a capsid comprising SEQ ID NO: 2871.

In some embodiments, the AAV particle comprises a viral genome comprising SEQ ID NO: 2199 and a VOY101 capsid. In some embodiments, the AAV particle comprises a viral genome comprising SEQ ID NO: 2199 and a VOY201 capsid. In some embodiments, the AAV particle comprises a viral genome comprising SEQ ID NO: 2199 and an AAV9 capsid. In some embodiments, the AAV particle comprises a viral genome comprising SEQ ID NO: 2199 and an AAV9K449R capsid. In some embodiments, the AAV particle comprises a viral genome comprising SEQ ID NO: 2199 and an AAVPHP.B capsid. In some embodiments, the AAV particle comprises a viral genome comprising SEQ ID NO: 2199 and an AAVPHP.N capsid. In some embodiments, the AAV particle comprises a viral genome comprising SEQ ID NO: 2199 and an AAV2 variant capsid. In some embodiments, the AAV particle comprises a viral genome comprising SEQ ID NO: 2199 and an AAV2/3 variant capsid. In some embodiments, the AAV particle comprises a viral genome comprising SEQ ID NO: 2199 and a capsid comprising SEQ ID NO: 1. In some embodiments, the AAV particle comprises a viral genome comprising SEQ ID NO: 2199 and a capsid encoded by a nucleic acid sequence comprising SEQ ID NO: 2. In some embodiments, the AAV particle comprises a viral genome comprising SEQ ID NO: 2199 and a capsid comprising SEQ ID NO: 4534. In some embodiments, the AAV particle comprises a viral genome comprising SEQ ID NO: 2199 and a capsid encoded by a nucleic acid sequence comprising SEQ ID NO: 3. In some embodiments, the AAV particle comprises a viral genome comprising SEQ ID NO: 2199 and a capsid comprising SEQ ID NO: 138. In some embodiments, the AAV particle comprises a viral genome comprising SEQ ID NO: 2199 and a capsid encoded by a nucleic acid sequence comprising SEQ ID NO: 137. In some embodiments, the AAV particle comprises a viral genome comprising SEQ ID NO: 2199 and a capsid comprising SEQ ID NO: 5. In some embodiments, the AAV particle comprises a viral genome comprising SEQ ID NO: 2199 and a capsid encoded by a nucleic acid sequence comprising SEQ ID NO: 6. In some embodiments, the AAV particle comprises a viral genome comprising SEQ ID NO: 2199 and a capsid comprising SEQ ID NO: 4. In some embodiments, the AAV particle comprises a viral genome comprising SEQ ID NO: 2199 and a capsid comprising SEQ ID NO: 11. In some embodiments, the AAV particle comprises a viral genome comprising SEQ ID NO: 2199 and a capsid comprising SEQ ID NO: 2679. In some embodiments, the AAV particle comprises a viral genome comprising SEQ ID NO: 2199 and a capsid comprising SEQ ID NO: 2809. In some embodiments, the AAV particle comprises a viral genome comprising SEQ ID NO: 2199 and a capsid comprising SEQ ID NO: 2871.

In some embodiments, the AAV particle comprises a viral genome comprising SEQ ID NO: 2200 and a VOY101 capsid. In some embodiments, the AAV particle comprises a viral genome comprising SEQ ID NO: 2200 and a VOY201 capsid. In some embodiments, the AAV particle comprises a viral genome comprising SEQ ID NO: 2200 and an AAV9 capsid. In some embodiments, the AAV particle comprises a viral genome comprising SEQ ID NO: 2200 and an AAV9K449R capsid. In some embodiments, the AAV particle comprises a viral genome comprising SEQ ID NO: 2200 and an AAVPHP.B capsid. In some embodiments, the AAV particle comprises a viral genome comprising SEQ ID NO: 2200 and an AAVPHP.N capsid. In some embodiments, the AAV particle comprises a viral genome comprising SEQ ID NO: 2200 and an AAV2 variant capsid. In some embodiments, the AAV particle comprises a viral genome comprising SEQ ID NO: 2200 and an AAV2/3 variant capsid. In some embodiments, the AAV particle comprises a viral genome comprising SEQ ID NO: 2200 and a capsid comprising SEQ ID NO: 1. In some embodiments, the AAV particle comprises a viral genome comprising SEQ ID NO: 2200 and a capsid encoded by a nucleic acid sequence comprising SEQ ID NO: 2. In some embodiments, the AAV particle comprises a viral genome comprising SEQ ID NO: 2200 and a capsid comprising SEQ ID NO: 4534. In some embodiments, the AAV particle comprises a viral genome comprising SEQ ID NO: 2200 and a capsid encoded by a nucleic acid sequence comprising SEQ ID NO: 3. In some embodiments, the AAV particle comprises a viral genome comprising SEQ ID NO: 2200 and a capsid comprising SEQ ID NO: 138. In some embodiments, the AAV particle comprises a viral genome comprising SEQ ID NO: 2200 and a capsid encoded by a nucleic acid sequence comprising SEQ ID NO: 137. In some embodiments, the AAV particle comprises a viral genome comprising SEQ ID NO: 2200 and a capsid comprising SEQ ID NO: 5. In some embodiments, the AAV particle comprises a viral genome comprising SEQ ID NO: 2200 and a capsid encoded by a nucleic acid sequence comprising SEQ ID NO: 6. In some embodiments, the AAV particle comprises a viral genome comprising SEQ ID NO: 2200 and a capsid comprising SEQ ID NO: 4. In some embodiments, the AAV particle comprises a viral genome comprising SEQ ID NO: 2200 and a capsid comprising SEQ ID NO: 11. In some embodiments, the AAV particle comprises a viral genome comprising SEQ ID NO: 2200 and a capsid comprising SEQ ID NO: 2679. In some embodiments, the AAV particle comprises a viral genome comprising SEQ ID NO: 2200 and a capsid comprising SEQ ID NO: 2809. In some embodiments, the AAV particle comprises a viral genome comprising SEQ ID NO: 2200 and a capsid comprising SEQ ID NO: 2871.

In some embodiments, the AAV particle comprises a viral genome comprising SEQ ID NO: 2201 and a VOY101 capsid. In some embodiments, the AAV particle comprises a viral genome comprising SEQ ID NO: 2201 and a VOY201 capsid. In some embodiments, the AAV particle comprises a viral genome comprising SEQ ID NO: 2201 and an AAV9 capsid. In some embodiments, the AAV particle comprises a viral genome comprising SEQ ID NO: 2201 and an AAV9K449R capsid. In some embodiments, the AAV particle comprises a viral genome comprising SEQ ID NO: 2201 and an AAVPHP.B capsid. In some embodiments, the AAV particle comprises a viral genome comprising SEQ ID NO: 2201 and an AAVPHP.N capsid. In some embodiments, the AAV particle comprises a viral genome comprising SEQ ID NO: 2201 and an AAV2 variant capsid. In some embodiments, the AAV particle comprises a viral genome comprising SEQ ID NO: 2201 and an AAV2/3 variant capsid. In some embodiments, the AAV particle comprises a viral genome comprising SEQ ID NO: 2201 and a capsid comprising SEQ ID NO: 1. In some embodiments, the AAV particle comprises a viral genome comprising SEQ ID NO: 2201 and a capsid encoded by a nucleic acid sequence comprising SEQ ID NO: 2. In some embodiments, the AAV particle comprises a viral genome comprising SEQ ID NO: 2201 and a capsid comprising SEQ ID NO: 4534. In some embodiments, the AAV particle comprises a viral genome comprising SEQ ID NO: 2201 and a capsid encoded by a nucleic acid sequence comprising SEQ ID NO: 3. In some embodiments, the AAV particle comprises a viral genome comprising SEQ ID NO: 2201 and a capsid comprising SEQ ID NO: 138. In some embodiments, the AAV particle comprises a viral genome comprising SEQ ID NO: 2201 and a capsid encoded by a nucleic acid sequence comprising SEQ ID NO: 137. In some embodiments, the AAV particle comprises a viral genome comprising SEQ ID NO: 2201 and a capsid comprising SEQ ID NO: 5. In some embodiments, the AAV particle comprises a viral genome comprising SEQ ID NO: 2201 and a capsid encoded by a nucleic acid sequence comprising SEQ ID NO: 6. In some embodiments, the AAV particle comprises a viral genome comprising SEQ ID NO: 2201 and a capsid comprising SEQ ID NO: 4. In some embodiments, the AAV particle comprises a viral genome comprising SEQ ID NO: 2201 and a capsid comprising SEQ ID NO: 11. In some embodiments, the AAV particle comprises a viral genome comprising SEQ ID NO: 2201 and a capsid comprising SEQ ID NO: 2679. In some embodiments, the AAV particle comprises a viral genome comprising SEQ ID NO: 2201 and a capsid comprising SEQ ID NO: 2809. In some embodiments, the AAV particle comprises a viral genome comprising SEQ ID NO: 2201 and a capsid comprising SEQ ID NO: 2871.

In some embodiments, the AAV particle comprises a viral genome comprising SEQ ID NO: 2202 and a VOY101 capsid. In some embodiments, the AAV particle comprises a viral genome comprising SEQ ID NO: 2202 and a VOY201 capsid. In some embodiments, the AAV particle comprises a viral genome comprising SEQ ID NO: 2202 and an AAV9 capsid. In some embodiments, the AAV particle comprises a viral genome comprising SEQ ID NO: 2202 and an AAV9K449R capsid. In some embodiments, the AAV particle comprises a viral genome comprising SEQ ID NO: 2202 and an AAVPHP.B capsid. In some embodiments, the AAV particle comprises a viral genome comprising SEQ ID NO: 2202 and an AAVPHP.N capsid. In some embodiments, the AAV particle comprises a viral genome comprising SEQ ID NO: 2202 and an AAV2 variant capsid. In some embodiments, the AAV particle comprises a viral genome comprising SEQ ID NO: 2202 and an AAV2/3 variant capsid. In some embodiments, the AAV particle comprises a viral genome comprising SEQ ID NO: 2202 and a capsid comprising SEQ ID NO: 1. In some embodiments, the AAV particle comprises a viral genome comprising SEQ ID NO: 2202 and a capsid encoded by a nucleic acid sequence comprising SEQ ID NO: 2. In some embodiments, the AAV particle comprises a viral genome comprising SEQ ID NO: 2202 and a capsid comprising SEQ ID NO: 4534. In some embodiments, the AAV particle comprises a viral genome comprising SEQ ID NO: 2202 and a capsid encoded by a nucleic acid sequence comprising SEQ ID NO: 3. In some embodiments, the AAV particle comprises a viral genome comprising SEQ ID NO: 2202 and a capsid comprising SEQ ID NO: 138. In some embodiments, the AAV particle comprises a viral genome comprising SEQ ID NO: 2202 and a capsid encoded by a nucleic acid sequence comprising SEQ ID NO: 137. In some embodiments, the AAV particle comprises a viral genome comprising SEQ ID NO: 2202 and a capsid comprising SEQ ID NO: 5. In some embodiments, the AAV particle comprises a viral genome comprising SEQ ID NO: 2202 and a capsid encoded by a nucleic acid sequence comprising SEQ ID NO: 6. In some embodiments, the AAV particle comprises a viral genome comprising SEQ ID NO: 2202 and a capsid comprising SEQ ID NO: 4. In some embodiments, the AAV particle comprises a viral genome comprising SEQ ID NO: 2202 and a capsid comprising SEQ ID NO: 11. In some embodiments, the AAV particle comprises a viral genome comprising SEQ ID NO: 2202 and a capsid comprising SEQ ID NO: 2679. In some embodiments, the AAV particle comprises a viral genome comprising SEQ ID NO: 2202 and a capsid comprising SEQ ID NO: 2809. In some embodiments, the AAV particle comprises a viral genome comprising SEQ ID NO: 2202 and a capsid comprising SEQ ID NO: 2871.

In some embodiments, the AAV particle comprises a viral genome comprising SEQ ID NO: 2203 and a VOY101 capsid. In some embodiments, the AAV particle comprises a viral genome comprising SEQ ID NO: 2203 and a VOY201 capsid. In some embodiments, the AAV particle comprises a viral genome comprising SEQ ID NO: 2203 and an AAV9 capsid. In some embodiments, the AAV particle comprises a viral genome comprising SEQ ID NO: 2203 and an AAV9K449R capsid. In some embodiments, the AAV particle comprises a viral genome comprising SEQ ID NO: 2203 and an AAVPHP.B capsid. In some embodiments, the AAV particle comprises a viral genome comprising SEQ ID NO: 2203 and an AAVPHP.N capsid. In some embodiments, the AAV particle comprises a viral genome comprising SEQ ID NO: 2203 and an AAV2 variant capsid. In some embodiments, the AAV particle comprises a viral genome comprising SEQ ID NO: 2203 and an AAV2/3 variant capsid. In some embodiments, the AAV particle comprises a viral genome comprising SEQ ID NO: 2203 and a capsid comprising SEQ ID NO: 1. In some embodiments, the AAV particle comprises a viral genome comprising SEQ ID NO: 2203 and a capsid encoded by a nucleic acid sequence comprising SEQ ID NO: 2. In some embodiments, the AAV particle comprises a viral genome comprising SEQ ID NO: 2203 and a capsid comprising SEQ ID NO: 4534. In some embodiments, the AAV particle comprises a viral genome comprising SEQ ID NO: 2203 and a capsid encoded by a nucleic acid sequence comprising SEQ ID NO: 3. In some embodiments, the AAV particle comprises a viral genome comprising SEQ ID NO: 2203 and a capsid comprising SEQ ID NO: 138. In some embodiments, the AAV particle comprises a viral genome comprising SEQ ID NO: 2203 and a capsid encoded by a nucleic acid sequence comprising SEQ ID NO: 137. In some embodiments, the AAV particle comprises a viral genome comprising SEQ ID NO: 2203 and a capsid comprising SEQ ID NO: 5. In some embodiments, the AAV particle comprises a viral genome comprising SEQ ID NO: 2203 and a capsid encoded by a nucleic acid sequence comprising SEQ ID NO: 6. In some embodiments, the AAV particle comprises a viral genome comprising SEQ ID NO: 2203 and a capsid comprising SEQ ID NO: 4. In some embodiments, the AAV particle comprises a viral genome comprising SEQ ID NO: 2203 and a capsid comprising SEQ ID NO: 11. In some embodiments, the AAV particle comprises a viral genome comprising SEQ ID NO: 2203 and a capsid comprising SEQ ID NO: 2679. In some embodiments, the AAV particle comprises a viral genome comprising SEQ ID NO: 2203 and a capsid comprising SEQ ID NO: 2809. In some embodiments, the AAV particle comprises a viral genome comprising SEQ ID NO: 2203 and a capsid comprising SEQ ID NO: 2871.

In some embodiments, the AAV particle comprises a viral genome comprising SEQ ID NO: 2204 and a VOY101 capsid. In some embodiments, the AAV particle comprises a viral genome comprising SEQ ID NO: 2204 and a VOY201 capsid. In some embodiments, the AAV particle comprises a viral genome comprising SEQ ID NO: 2204 and an AAV9 capsid. In some embodiments, the AAV particle comprises a viral genome comprising SEQ ID NO: 2204 and an AAV9K449R capsid. In some embodiments, the AAV particle comprises a viral genome comprising SEQ ID NO: 2204 and an AAVPHP.B capsid. In some embodiments, the AAV particle comprises a viral genome comprising SEQ ID NO: 2204 and an AAVPHP.N capsid. In some embodiments, the AAV particle comprises a viral genome comprising SEQ ID NO: 2204 and an AAV2 variant capsid. In some embodiments, the AAV particle comprises a viral genome comprising SEQ ID NO: 2204 and an AAV2/3 variant capsid. In some embodiments, the AAV particle comprises a viral genome comprising SEQ ID NO: 2204 and a capsid comprising SEQ ID NO: 1. In some embodiments, the AAV particle comprises a viral genome comprising SEQ ID NO: 2204 and a capsid encoded by a nucleic acid sequence comprising SEQ ID NO: 2. In some embodiments, the AAV particle comprises a viral genome comprising SEQ ID NO: 2204 and a capsid comprising SEQ ID NO: 4534. In some embodiments, the AAV particle comprises a viral genome comprising SEQ ID NO: 2204 and a capsid encoded by a nucleic acid sequence comprising SEQ ID NO: 3. In some embodiments, the AAV particle comprises a viral genome comprising SEQ ID NO: 2204 and a capsid comprising SEQ ID NO: 138. In some embodiments, the AAV particle comprises a viral genome comprising SEQ ID NO: 2204 and a capsid encoded by a nucleic acid sequence comprising SEQ ID NO: 137. In some embodiments, the AAV particle comprises a viral genome comprising SEQ ID NO: 2204 and a capsid comprising SEQ ID NO: 5. In some embodiments, the AAV particle comprises a viral genome comprising SEQ ID NO: 2204 and a capsid encoded by a nucleic acid sequence comprising SEQ ID NO: 6. In some embodiments, the AAV particle comprises a viral genome comprising SEQ ID NO: 2204 and a capsid comprising SEQ ID NO: 4. In some embodiments, the AAV particle comprises a viral genome comprising SEQ ID NO: 2204 and a capsid comprising SEQ ID NO: 11. In some embodiments, the AAV particle comprises a viral genome comprising SEQ ID NO: 2204 and a capsid comprising SEQ ID NO: 2679. In some embodiments, the AAV particle comprises a viral genome comprising SEQ ID NO: 2204 and a capsid comprising SEQ ID NO: 2809. In some embodiments, the AAV particle comprises a viral genome comprising SEQ ID NO: 2204 and a capsid comprising SEQ ID NO: 2871.

In some embodiments, the AAV particle comprises a viral genome comprising SEQ ID NO: 2205 and a VOY101 capsid. In some embodiments, the AAV particle comprises a viral genome comprising SEQ ID NO: 2205 and a VOY201 capsid. In some embodiments, the AAV particle comprises a viral genome comprising SEQ ID NO: 2205 and an AAV9 capsid. In some embodiments, the AAV particle comprises a viral genome comprising SEQ ID NO: 2205 and an AAV9K449R capsid. In some embodiments, the AAV particle comprises a viral genome comprising SEQ ID NO: 2205 and an AAVPHP.B capsid. In some embodiments, the AAV particle comprises a viral genome comprising SEQ ID NO: 2205 and an AAVPHP.N capsid. In some embodiments, the AAV particle comprises a viral genome comprising SEQ ID NO: 2205 and an AAV2 variant capsid. In some embodiments, the AAV particle comprises a viral genome comprising SEQ ID NO: 2205 and an AAV2/3 variant capsid. In some embodiments, the AAV particle comprises a viral genome comprising SEQ ID NO: 2205 and a capsid comprising SEQ ID NO: 1. In some embodiments, the AAV particle comprises a viral genome comprising SEQ ID NO: 2205 and a capsid encoded by a nucleic acid sequence comprising SEQ ID NO: 2. In some embodiments, the AAV particle comprises a viral genome comprising SEQ ID NO: 2205 and a capsid comprising SEQ ID NO: 4534. In some embodiments, the AAV particle comprises a viral genome comprising SEQ ID NO: 2205 and a capsid encoded by a nucleic acid sequence comprising SEQ ID NO: 3. In some embodiments, the AAV particle comprises a viral genome comprising SEQ ID NO: 2205 and a capsid comprising SEQ ID NO: 138. In some embodiments, the AAV particle comprises a viral genome comprising SEQ ID NO: 2205 and a capsid encoded by a nucleic acid sequence comprising SEQ ID NO: 137. In some embodiments, the AAV particle comprises a viral genome comprising SEQ ID NO: 2205 and a capsid comprising SEQ ID NO: 5. In some embodiments, the AAV particle comprises a viral genome comprising SEQ ID NO: 2205 and a capsid encoded by a nucleic acid sequence comprising SEQ ID NO: 6. In some embodiments, the AAV particle comprises a viral genome comprising SEQ ID NO: 2205 and a capsid comprising SEQ ID NO: 4. In some embodiments, the AAV particle comprises a viral genome comprising SEQ ID NO: 2205 and a capsid comprising SEQ ID NO: 11. In some embodiments, the AAV particle comprises a viral genome comprising SEQ ID NO: 2205 and a capsid comprising SEQ ID NO: 2679. In some embodiments, the AAV particle comprises a viral genome comprising SEQ ID NO: 2205 and a capsid comprising SEQ ID NO: 2809. In some embodiments, the AAV particle comprises a viral genome comprising SEQ ID NO: 2205 and a capsid comprising SEQ ID NO: 2871.

In some embodiments, the AAV particle comprises a viral genome comprising SEQ ID NO: 2206 and a VOY101 capsid. In some embodiments, the AAV particle comprises a viral genome comprising SEQ ID NO: 2206 and a VOY201 capsid. In some embodiments, the AAV particle comprises a viral genome comprising SEQ ID NO: 2206 and an AAV9 capsid. In some embodiments, the AAV particle comprises a viral genome comprising SEQ ID NO: 2206 and an AAV9K449R capsid. In some embodiments, the AAV particle comprises a viral genome comprising SEQ ID NO: 2206 and an AAVPHP.B capsid. In some embodiments, the AAV particle comprises a viral genome comprising SEQ ID NO: 2206 and an AAVPHP.N capsid. In some embodiments, the AAV particle comprises a viral genome comprising SEQ ID NO: 2206 and an AAV2 variant capsid. In some embodiments, the AAV particle comprises a viral genome comprising SEQ ID NO: 2206 and an AAV2/3 variant capsid. In some embodiments, the AAV particle comprises a viral genome comprising SEQ ID NO: 2206 and a capsid comprising SEQ ID NO: 1. In some embodiments, the AAV particle comprises a viral genome comprising SEQ ID NO: 2206 and a capsid encoded by a nucleic acid sequence comprising SEQ ID NO: 2. In some embodiments, the AAV particle comprises a viral genome comprising SEQ ID NO: 2206 and a capsid comprising SEQ ID NO: 4534. In some embodiments, the AAV particle comprises a viral genome comprising SEQ ID NO: 2206 and a capsid encoded by a nucleic acid sequence comprising SEQ ID NO: 3. In some embodiments, the AAV particle comprises a viral genome comprising SEQ ID NO: 2206 and a capsid comprising SEQ ID NO: 138. In some embodiments, the AAV particle comprises a viral genome comprising SEQ ID NO: 2206 and a capsid encoded by a nucleic acid sequence comprising SEQ ID NO: 137. In some embodiments, the AAV particle comprises a viral genome comprising SEQ ID NO: 2206 and a capsid comprising SEQ ID NO: 5. In some embodiments, the AAV particle comprises a viral genome comprising SEQ ID NO: 2206 and a capsid encoded by a nucleic acid sequence comprising SEQ ID NO: 6. In some embodiments, the AAV particle comprises a viral genome comprising SEQ ID NO: 2206 and a capsid comprising SEQ ID NO: 4. In some embodiments, the AAV particle comprises a viral genome comprising SEQ ID NO: 2206 and a capsid comprising SEQ ID NO: 11. In some embodiments, the AAV particle comprises a viral genome comprising SEQ ID NO: 2206 and a capsid comprising SEQ ID NO: 2679. In some embodiments, the AAV particle comprises a viral genome comprising SEQ ID NO: 2206 and a capsid comprising SEQ ID NO: 2809. In some embodiments, the AAV particle comprises a viral genome comprising SEQ ID NO: 2206 and a capsid comprising SEQ ID NO: 2871.

In some embodiments, the AAV particle comprises a viral genome comprising SEQ ID NO: 2207 and a VOY101 capsid. In some embodiments, the AAV particle comprises a viral genome comprising SEQ ID NO: 2207 and a VOY201 capsid. In some embodiments, the AAV particle comprises a viral genome comprising SEQ ID NO: 2207 and an AAV9 capsid. In some embodiments, the AAV particle comprises a viral genome comprising SEQ ID NO: 2207 and an AAV9K449R capsid. In some embodiments, the AAV particle comprises a viral genome comprising SEQ ID NO: 2207 and an AAVPHP.B capsid. In some embodiments, the AAV particle comprises a viral genome comprising SEQ ID NO: 2207 and an AAVPHP.N capsid. In some embodiments, the AAV particle comprises a viral genome comprising SEQ ID NO: 2207 and an AAV2 variant capsid. In some embodiments, the AAV particle comprises a viral genome comprising SEQ ID NO: 2207 and an AAV2/3 variant capsid. In some embodiments, the AAV particle comprises a viral genome comprising SEQ ID NO: 2207 and a capsid comprising SEQ ID NO: 1. In some embodiments, the AAV particle comprises a viral genome comprising SEQ ID NO: 2207 and a capsid encoded by a nucleic acid sequence comprising SEQ ID NO: 2. In some embodiments, the AAV particle comprises a viral genome comprising SEQ ID NO: 2207 and a capsid comprising SEQ ID NO: 4534. In some embodiments, the AAV particle comprises a viral genome comprising SEQ ID NO: 2207 and a capsid encoded by a nucleic acid sequence comprising SEQ ID NO: 3. In some embodiments, the AAV particle comprises a viral genome comprising SEQ ID NO: 2207 and a capsid comprising SEQ ID NO: 138. In some embodiments, the AAV particle comprises a viral genome comprising SEQ ID NO: 2207 and a capsid encoded by a nucleic acid sequence comprising SEQ ID NO: 137. In some embodiments, the AAV particle comprises a viral genome comprising SEQ ID NO: 2207 and a capsid comprising SEQ ID NO: 5. In some embodiments, the AAV particle comprises a viral genome comprising SEQ ID NO: 2207 and a capsid encoded by a nucleic acid sequence comprising SEQ ID NO: 6. In some embodiments, the AAV particle comprises a viral genome comprising SEQ ID NO: 2207 and a capsid comprising SEQ ID NO: 4. In some embodiments, the AAV particle comprises a viral genome comprising SEQ ID NO: 2207 and a capsid comprising SEQ ID NO: 11. In some embodiments, the AAV particle comprises a viral genome comprising SEQ ID NO: 2207 and a capsid comprising SEQ ID NO: 2679. In some embodiments, the AAV particle comprises a viral genome comprising SEQ ID NO: 2207 and a capsid comprising SEQ ID NO: 2809. In some embodiments, the AAV particle comprises a viral genome comprising SEQ ID NO: 2207 and a capsid comprising SEQ ID NO: 2871.

In some embodiments, the AAV particle comprises a viral genome comprising SEQ ID NO: 2208 and a VOY101 capsid. In some embodiments, the AAV particle comprises a viral genome comprising SEQ ID NO: 2208 and a VOY201 capsid. In some embodiments, the AAV particle comprises a viral genome comprising SEQ ID NO: 2208 and an AAV9 capsid. In some embodiments, the AAV particle comprises a viral genome comprising SEQ ID NO: 2208 and an AAV9K449R capsid. In some embodiments, the AAV particle comprises a viral genome comprising SEQ ID NO: 2208 and an AAVPHP.B capsid. In some embodiments, the AAV particle comprises a viral genome comprising SEQ ID NO: 2208 and an AAVPHP.N capsid. In some embodiments, the AAV particle comprises a viral genome comprising SEQ ID NO: 2208 and an AAV2 variant capsid. In some embodiments, the AAV particle comprises a viral genome comprising SEQ ID NO: 2208 and an AAV2/3 variant capsid. In some embodiments, the AAV particle comprises a viral genome comprising SEQ ID NO: 2208 and a capsid comprising SEQ ID NO: 1. In some embodiments, the AAV particle comprises a viral genome comprising SEQ ID NO: 2208 and a capsid encoded by a nucleic acid sequence comprising SEQ ID NO: 2. In some embodiments, the AAV particle comprises a viral genome comprising SEQ ID NO: 2208 and a capsid comprising SEQ ID NO: 4534. In some embodiments, the AAV particle comprises a viral genome comprising SEQ ID NO: 2208 and a capsid encoded by a nucleic acid sequence comprising SEQ ID NO: 3. In some embodiments, the AAV particle comprises a viral genome comprising SEQ ID NO: 2208 and a capsid comprising SEQ ID NO: 138. In some embodiments, the AAV particle comprises a viral genome comprising SEQ ID NO: 2208 and a capsid encoded by a nucleic acid sequence comprising SEQ ID NO: 137. In some embodiments, the AAV particle comprises a viral genome comprising SEQ ID NO: 2208 and a capsid comprising SEQ ID NO: 5. In some embodiments, the AAV particle comprises a viral genome comprising SEQ ID NO: 2208 and a capsid encoded by a nucleic acid sequence comprising SEQ ID NO: 6. In some embodiments, the AAV particle comprises a viral genome comprising SEQ ID NO: 2208 and a capsid comprising SEQ ID NO: 4. In some embodiments, the AAV particle comprises a viral genome comprising SEQ ID NO: 2208 and a capsid comprising SEQ ID NO: 11. In some embodiments, the AAV particle comprises a viral genome comprising SEQ ID NO: 2208 and a capsid comprising SEQ ID NO: 2679. In some embodiments, the AAV particle comprises a viral genome comprising SEQ ID NO: 2208 and a capsid comprising SEQ ID NO: 2809. In some embodiments, the AAV particle comprises a viral genome comprising SEQ ID NO: 2208 and a capsid comprising SEQ ID NO: 2871.

In some embodiments, the AAV particle comprises a viral genome comprising SEQ ID NO: 2209 and a VOY101 capsid. In some embodiments, the AAV particle comprises a viral genome comprising SEQ ID NO: 2209 and a VOY201 capsid. In some embodiments, the AAV particle comprises a viral genome comprising SEQ ID NO: 2209 and an AAV9 capsid. In some embodiments, the AAV particle comprises a viral genome comprising SEQ ID NO: 2209 and an AAV9K449R capsid. In some embodiments, the AAV particle comprises a viral genome comprising SEQ ID NO: 2209 and an AAVPHP.B capsid. In some embodiments, the AAV particle comprises a viral genome comprising SEQ ID NO: 2209 and an AAVPHP.N capsid. In some embodiments, the AAV particle comprises a viral genome comprising SEQ ID NO: 2209 and an AAV2 variant capsid. In some embodiments, the AAV particle comprises a viral genome comprising SEQ ID NO: 2209 and an AAV2/3 variant capsid. In some embodiments, the AAV particle comprises a viral genome comprising SEQ ID NO: 2209 and a capsid comprising SEQ ID NO: 1. In some embodiments, the AAV particle comprises a viral genome comprising SEQ ID NO: 2209 and a capsid encoded by a nucleic acid sequence comprising SEQ ID NO: 2. In some embodiments, the AAV particle comprises a viral genome comprising SEQ ID NO: 2209 and a capsid comprising SEQ ID NO: 4534. In some embodiments, the AAV particle comprises a viral genome comprising SEQ ID NO: 2209 and a capsid encoded by a nucleic acid sequence comprising SEQ ID NO: 3. In some embodiments, the AAV particle comprises a viral genome comprising SEQ ID NO: 2209 and a capsid comprising SEQ ID NO: 138. In some embodiments, the AAV particle comprises a viral genome comprising SEQ ID NO: 2209 and a capsid encoded by a nucleic acid sequence comprising SEQ ID NO: 137. In some embodiments, the AAV particle comprises a viral genome comprising SEQ ID NO: 2209 and a capsid comprising SEQ ID NO: 5. In some embodiments, the AAV particle comprises a viral genome comprising SEQ ID NO: 2209 and a capsid encoded by a nucleic acid sequence comprising SEQ ID NO: 6. In some embodiments, the AAV particle comprises a viral genome comprising SEQ ID NO: 2209 and a capsid comprising SEQ ID NO: 4. In some embodiments, the AAV particle comprises a viral genome comprising SEQ ID NO: 2209 and a capsid comprising SEQ ID NO: 11. In some embodiments, the AAV particle comprises a viral genome comprising SEQ ID NO: 2209 and a capsid comprising SEQ ID NO: 2679. In some embodiments, the AAV particle comprises a viral genome comprising SEQ ID NO: 2209 and a capsid comprising SEQ ID NO: 2809. In some embodiments, the AAV particle comprises a viral genome comprising SEQ ID NO: 2209 and a capsid comprising SEQ ID NO: 2871.

In some embodiments, the AAV particle comprises a viral genome comprising SEQ ID NO: 2210 and a VOY101 capsid. In some embodiments, the AAV particle comprises a viral genome comprising SEQ ID NO: 2210 and a VOY201 capsid. In some embodiments, the AAV particle comprises a viral genome comprising SEQ ID NO: 2210 and an AAV9 capsid. In some embodiments, the AAV particle comprises a viral genome comprising SEQ ID NO: 2210 and an AAV9K449R capsid. In some embodiments, the AAV particle comprises a viral genome comprising SEQ ID NO: 2210 and an AAVPHP.B capsid. In some embodiments, the AAV particle comprises a viral genome comprising SEQ ID NO: 2210 and an AAVPHP.N capsid. In some embodiments, the AAV particle comprises a viral genome comprising SEQ ID NO: 2210 and an AAV2 variant capsid. In some embodiments, the AAV particle comprises a viral genome comprising SEQ ID NO: 2210 and an AAV2/3 variant capsid. In some embodiments, the AAV particle comprises a viral genome comprising SEQ ID NO: 2210 and a capsid comprising SEQ ID NO: 1. In some embodiments, the AAV particle comprises a viral genome comprising SEQ ID NO: 2210 and a capsid encoded by a nucleic acid sequence comprising SEQ ID NO: 2. In some embodiments, the AAV particle comprises a viral genome comprising SEQ ID NO: 2210 and a capsid comprising SEQ ID NO: 4534. In some embodiments, the AAV particle comprises a viral genome comprising SEQ ID NO: 2210 and a capsid encoded by a nucleic acid sequence comprising SEQ ID NO: 3. In some embodiments, the AAV particle comprises a viral genome comprising SEQ ID NO: 2210 and a capsid comprising SEQ ID NO: 138. In some embodiments, the AAV particle comprises a viral genome comprising SEQ ID NO: 2210 and a capsid encoded by a nucleic acid sequence comprising SEQ ID NO: 137. In some embodiments, the AAV particle comprises a viral genome comprising SEQ ID NO: 2210 and a capsid comprising SEQ ID NO: 5. In some embodiments, the AAV particle comprises a viral genome comprising SEQ ID NO: 2210 and a capsid encoded by a nucleic acid sequence comprising SEQ ID NO: 6. In some embodiments, the AAV particle comprises a viral genome comprising SEQ ID NO: 2210 and a capsid comprising SEQ ID NO: 4. In some embodiments, the AAV particle comprises a viral genome comprising SEQ ID NO: 2210 and a capsid comprising SEQ ID NO: 11. In some embodiments, the AAV particle comprises a viral genome comprising SEQ ID NO: 2210 and a capsid comprising SEQ ID NO: 2679. In some embodiments, the AAV particle comprises a viral genome comprising SEQ ID NO: 2210 and a capsid comprising SEQ ID NO: 2809. In some embodiments, the AAV particle comprises a viral genome comprising SEQ ID NO: 2210 and a capsid comprising SEQ ID NO: 2871.

In some embodiments, the AAV particle comprises a viral genome comprising SEQ ID NO: 2211 and a VOY101 capsid. In some embodiments, the AAV particle comprises a viral genome comprising SEQ ID NO: 2211 and a VOY201 capsid. In some embodiments, the AAV particle comprises a viral genome comprising SEQ ID NO: 2211 and an AAV9 capsid. In some embodiments, the AAV particle comprises a viral genome comprising SEQ ID NO: 2211 and an AAV9K449R capsid. In some embodiments, the AAV particle comprises a viral genome comprising SEQ ID NO: 2211 and an AAVPHP.B capsid. In some embodiments, the AAV particle comprises a viral genome comprising SEQ ID NO: 2211 and an AAVPHP.N capsid. In some embodiments, the AAV particle comprises a viral genome comprising SEQ ID NO: 2211 and an AAV2 variant capsid. In some embodiments, the AAV particle comprises a viral genome comprising SEQ ID NO: 2211 and an AAV2/3 variant capsid. In some embodiments, the AAV particle comprises a viral genome comprising SEQ ID NO: 2211 and a capsid comprising SEQ ID NO: 1. In some embodiments, the AAV particle comprises a viral genome comprising SEQ ID NO: 2211 and a capsid encoded by a nucleic acid sequence comprising SEQ ID NO: 2. In some embodiments, the AAV particle comprises a viral genome comprising SEQ ID NO: 2211 and a capsid comprising SEQ ID NO: 4534. In some embodiments, the AAV particle comprises a viral genome comprising SEQ ID NO: 2211 and a capsid encoded by a nucleic acid sequence comprising SEQ ID NO: 3. In some embodiments, the AAV particle comprises a viral genome comprising SEQ ID NO: 2211 and a capsid comprising SEQ ID NO: 138. In some embodiments, the AAV particle comprises a viral genome comprising SEQ ID NO: 2211 and a capsid encoded by a nucleic acid sequence comprising SEQ ID NO: 137. In some embodiments, the AAV particle comprises a viral genome comprising SEQ ID NO: 2211 and a capsid comprising SEQ ID NO: 5. In some embodiments, the AAV particle comprises a viral genome comprising SEQ ID NO: 2211 and a capsid encoded by a nucleic acid sequence comprising SEQ ID NO: 6. In some embodiments, the AAV particle comprises a viral genome comprising SEQ ID NO: 2211 and a capsid comprising SEQ ID NO: 4. In some embodiments, the AAV particle comprises a viral genome comprising SEQ ID NO: 2211 and a capsid comprising SEQ ID NO: 11. In some embodiments, the AAV particle comprises a viral genome comprising SEQ ID NO: 2211 and a capsid comprising SEQ ID NO: 2679. In some embodiments, the AAV particle comprises a viral genome comprising SEQ ID NO: 2211 and a capsid comprising SEQ ID NO: 2809. In some embodiments, the AAV particle comprises a viral genome comprising SEQ ID NO: 2211 and a capsid comprising SEQ ID NO: 2871.

In some embodiments, the AAV particle comprises a viral genome comprising SEQ ID NO: 2212 and a VOY101 capsid. In some embodiments, the AAV particle comprises a viral genome comprising SEQ ID NO: 2212 and a VOY201 capsid. In some embodiments, the AAV particle comprises a viral genome comprising SEQ ID NO: 2212 and an AAV9 capsid. In some embodiments, the AAV particle comprises a viral genome comprising SEQ ID NO: 2212 and an AAV9K449R capsid. In some embodiments, the AAV particle comprises a viral genome comprising SEQ ID NO: 2212 and an AAVPHP.B capsid. In some embodiments, the AAV particle comprises a viral genome comprising SEQ ID NO: 2212 and an AAVPHP.N capsid. In some embodiments, the AAV particle comprises a viral genome comprising SEQ ID NO: 2212 and an AAV2 variant capsid. In some embodiments, the AAV particle comprises a viral genome comprising SEQ ID NO: 2212 and an AAV2/3 variant capsid. In some embodiments, the AAV particle comprises a viral genome comprising SEQ ID NO: 2212 and a capsid comprising SEQ ID NO: 1. In some embodiments, the AAV particle comprises a viral genome comprising SEQ ID NO: 2212 and a capsid encoded by a nucleic acid sequence comprising SEQ ID NO: 2. In some embodiments, the AAV particle comprises a viral genome comprising SEQ ID NO: 2212 and a capsid comprising SEQ ID NO: 4534. In some embodiments, the AAV particle comprises a viral genome comprising SEQ ID NO: 2212 and a capsid encoded by a nucleic acid sequence comprising SEQ ID NO: 3. In some embodiments, the AAV particle comprises a viral genome comprising SEQ ID NO: 2212 and a capsid comprising SEQ ID NO: 138. In some embodiments, the AAV particle comprises a viral genome comprising SEQ ID NO: 2212 and a capsid encoded by a nucleic acid sequence comprising SEQ ID NO: 137. In some embodiments, the AAV particle comprises a viral genome comprising SEQ ID NO: 2212 and a capsid comprising SEQ ID NO: 5. In some embodiments, the AAV particle comprises a viral genome comprising SEQ ID NO: 2212 and a capsid encoded by a nucleic acid sequence comprising SEQ ID NO: 6. In some embodiments, the AAV particle comprises a viral genome comprising SEQ ID NO: 2212 and a capsid comprising SEQ ID NO: 4. In some embodiments, the AAV particle comprises a viral genome comprising SEQ ID NO: 2212 and a capsid comprising SEQ ID NO: 11. In some embodiments, the AAV particle comprises a viral genome comprising SEQ ID NO: 2212 and a capsid comprising SEQ ID NO: 2679. In some embodiments, the AAV particle comprises a viral genome comprising SEQ ID NO: 2212 and a capsid comprising SEQ ID NO: 2809. In some embodiments, the AAV particle comprises a viral genome comprising SEQ ID NO: 2212 and a capsid comprising SEQ ID NO: 2871.

In some embodiments, the AAV particle comprises a viral genome comprising SEQ ID NO: 2213 and a VOY101 capsid. In some embodiments, the AAV particle comprises a viral genome comprising SEQ ID NO: 2213 and a VOY201 capsid. In some embodiments, the AAV particle comprises a viral genome comprising SEQ ID NO: 2213 and an AAV9 capsid. In some embodiments, the AAV particle comprises a viral genome comprising SEQ ID NO: 2213 and an AAV9K449R capsid. In some embodiments, the AAV particle comprises a viral genome comprising SEQ ID NO: 2213 and an AAVPHP.B capsid. In some embodiments, the AAV particle comprises a viral genome comprising SEQ ID NO: 2213 and an AAVPHP.N capsid. In some embodiments, the AAV particle comprises a viral genome comprising SEQ ID NO: 2213 and an AAV2 variant capsid. In some embodiments, the AAV particle comprises a viral genome comprising SEQ ID NO: 2213 and an AAV2/3 variant capsid. In some embodiments, the AAV particle comprises a viral genome comprising SEQ ID NO: 2213 and a capsid comprising SEQ ID NO: 1. In some embodiments, the AAV particle comprises a viral genome comprising SEQ ID NO: 2213 and a capsid encoded by a nucleic acid sequence comprising SEQ ID NO: 2. In some embodiments, the AAV particle comprises a viral genome comprising SEQ ID NO: 2213 and a capsid comprising SEQ ID NO: 4534. In some embodiments, the AAV particle comprises a viral genome comprising SEQ ID NO: 2213 and a capsid encoded by a nucleic acid sequence comprising SEQ ID NO: 3. In some embodiments, the AAV particle comprises a viral genome comprising SEQ ID NO: 2213 and a capsid comprising SEQ ID NO: 138. In some embodiments, the AAV particle comprises a viral genome comprising SEQ ID NO: 2213 and a capsid encoded by a nucleic acid sequence comprising SEQ ID NO: 137. In some embodiments, the AAV particle comprises a viral genome comprising SEQ ID NO: 2213 and a capsid comprising SEQ ID NO: 5. In some embodiments, the AAV particle comprises a viral genome comprising SEQ ID NO: 2213 and a capsid encoded by a nucleic acid sequence comprising SEQ ID NO: 6. In some embodiments, the AAV particle comprises a viral genome comprising SEQ ID NO: 2213 and a capsid comprising SEQ ID NO: 4. In some embodiments, the AAV particle comprises a viral genome comprising SEQ ID NO: 2213 and a capsid comprising SEQ ID NO: 11. In some embodiments, the AAV particle comprises a viral genome comprising SEQ ID NO: 2213 and a capsid comprising SEQ ID NO: 2679. In some embodiments, the AAV particle comprises a viral genome comprising SEQ ID NO: 2213 and a capsid comprising SEQ ID NO: 2809. In some embodiments, the AAV particle comprises a viral genome comprising SEQ ID NO: 2213 and a capsid comprising SEQ ID NO: 2871.

In some embodiments, the AAV particle comprises a viral genome comprising SEQ ID NO: 2214 and a VOY101 capsid. In some embodiments, the AAV particle comprises a viral genome comprising SEQ ID NO: 2214 and a VOY201 capsid. In some embodiments, the AAV particle comprises a viral genome comprising SEQ ID NO: 2214 and an AAV9 capsid. In some embodiments, the AAV particle comprises a viral genome comprising SEQ ID NO: 2214 and an AAV9K449R capsid. In some embodiments, the AAV particle comprises a viral genome comprising SEQ ID NO: 2214 and an AAVPHP.B capsid. In some embodiments, the AAV particle comprises a viral genome comprising SEQ ID NO: 2214 and an AAVPHP.N capsid. In some embodiments, the AAV particle comprises a viral genome comprising SEQ ID NO: 2214 and an AAV2 variant capsid. In some embodiments, the AAV particle comprises a viral genome comprising SEQ ID NO: 2214 and an AAV2/3 variant capsid. In some embodiments, the AAV particle comprises a viral genome comprising SEQ ID NO: 2214 and a capsid comprising SEQ ID NO: 1. In some embodiments, the AAV particle comprises a viral genome comprising SEQ ID NO: 2214 and a capsid encoded by a nucleic acid sequence comprising SEQ ID NO: 2. In some embodiments, the AAV particle comprises a viral genome comprising SEQ ID NO: 2214 and a capsid comprising SEQ ID NO: 4534. In some embodiments, the AAV particle comprises a viral genome comprising SEQ ID NO: 2214 and a capsid encoded by a nucleic acid sequence comprising SEQ ID NO: 3. In some embodiments, the AAV particle comprises a viral genome comprising SEQ ID NO: 2214 and a capsid comprising SEQ ID NO: 138. In some embodiments, the AAV particle comprises a viral genome comprising SEQ ID NO: 2214 and a capsid encoded by a nucleic acid sequence comprising SEQ ID NO: 137. In some embodiments, the AAV particle comprises a viral genome comprising SEQ ID NO: 2214 and a capsid comprising SEQ ID NO: 5. In some embodiments, the AAV particle comprises a viral genome comprising SEQ ID NO: 2214 and a capsid encoded by a nucleic acid sequence comprising SEQ ID NO: 6. In some embodiments, the AAV particle comprises a viral genome comprising SEQ ID NO: 2214 and a capsid comprising SEQ ID NO: 4. In some embodiments, the AAV particle comprises a viral genome comprising SEQ ID NO: 2214 and a capsid comprising SEQ ID NO: 11. In some embodiments, the AAV particle comprises a viral genome comprising SEQ ID NO: 2214 and a capsid comprising SEQ ID NO: 2679. In some embodiments, the AAV particle comprises a viral genome comprising SEQ ID NO: 2214 and a capsid comprising SEQ ID NO: 2809. In some embodiments, the AAV particle comprises a viral genome comprising SEQ ID NO: 2214 and a capsid comprising SEQ ID NO: 2871.

In some embodiments, the AAV particle comprises a viral genome comprising SEQ ID NO: 2215 and a VOY101 capsid. In some embodiments, the AAV particle comprises a viral genome comprising SEQ ID NO: 2215 and a VOY201 capsid. In some embodiments, the AAV particle comprises a viral genome comprising SEQ ID NO: 2215 and an AAV9 capsid. In some embodiments, the AAV particle comprises a viral genome comprising SEQ ID NO: 2215 and an AAV9K449R capsid. In some embodiments, the AAV particle comprises a viral genome comprising SEQ ID NO: 2215 and an AAVPHP.B capsid. In some embodiments, the AAV particle comprises a viral genome comprising SEQ ID NO: 2215 and an AAVPHP.N capsid. In some embodiments, the AAV particle comprises a viral genome comprising SEQ ID NO: 2215 and an AAV2 variant capsid. In some embodiments, the AAV particle comprises a viral genome comprising SEQ ID NO: 2215 and an AAV2/3 variant capsid. In some embodiments, the AAV particle comprises a viral genome comprising SEQ ID NO: 2215 and a capsid comprising SEQ ID NO: 1. In some embodiments, the AAV particle comprises a viral genome comprising SEQ ID NO: 2215 and a capsid encoded by a nucleic acid sequence comprising SEQ ID NO: 2. In some embodiments, the AAV particle comprises a viral genome comprising SEQ ID NO: 2215 and a capsid comprising SEQ ID NO: 4534. In some embodiments, the AAV particle comprises a viral genome comprising SEQ ID NO: 2215 and a capsid encoded by a nucleic acid sequence comprising SEQ ID NO: 3. In some embodiments, the AAV particle comprises a viral genome comprising SEQ ID NO: 2215 and a capsid comprising SEQ ID NO: 138. In some embodiments, the AAV particle comprises a viral genome comprising SEQ ID NO: 2215 and a capsid encoded by a nucleic acid sequence comprising SEQ ID NO: 137. In some embodiments, the AAV particle comprises a viral genome comprising SEQ ID NO: 2215 and a capsid comprising SEQ ID NO: 5. In some embodiments, the AAV particle comprises a viral genome comprising SEQ ID NO: 2215 and a capsid encoded by a nucleic acid sequence comprising SEQ ID NO: 6. In some embodiments, the AAV particle comprises a viral genome comprising SEQ ID NO: 2215 and a capsid comprising SEQ ID NO: 4. In some embodiments, the AAV particle comprises a viral genome comprising SEQ ID NO: 2215 and a capsid comprising SEQ ID NO: 11. In some embodiments, the AAV particle comprises a viral genome comprising SEQ ID NO: 2215 and a capsid comprising SEQ ID NO: 2679. In some embodiments, the AAV particle comprises a viral genome comprising SEQ ID NO: 2215 and a capsid comprising SEQ ID NO: 2809. In some embodiments, the AAV particle comprises a viral genome comprising SEQ ID NO: 2215 and a capsid comprising SEQ ID NO: 2871.

In some embodiments, the AAV particle comprises a viral genome comprising SEQ ID NO: 2216 and a VOY101 capsid. In some embodiments, the AAV particle comprises a viral genome comprising SEQ ID NO: 2216 and a VOY201 capsid. In some embodiments, the AAV particle comprises a viral genome comprising SEQ ID NO: 2216 and an AAV9 capsid. In some embodiments, the AAV particle comprises a viral genome comprising SEQ ID NO: 2216 and an AAV9K449R capsid. In some embodiments, the AAV particle comprises a viral genome comprising SEQ ID NO: 2216 and an AAVPHP.B capsid. In some embodiments, the AAV particle comprises a viral genome comprising SEQ ID NO: 2216 and an AAVPHP.N capsid. In some embodiments, the AAV particle comprises a viral genome comprising SEQ ID NO: 2216 and an AAV2 variant capsid. In some embodiments, the AAV particle comprises a viral genome comprising SEQ ID NO: 2216 and an AAV2/3 variant capsid. In some embodiments, the AAV particle comprises a viral genome comprising SEQ ID NO: 2216 and a capsid comprising SEQ ID NO: 1. In some embodiments, the AAV particle comprises a viral genome comprising SEQ ID NO: 2216 and a capsid encoded by a nucleic acid sequence comprising SEQ ID NO: 2. In some embodiments, the AAV particle comprises a viral genome comprising SEQ ID NO: 2216 and a capsid comprising SEQ ID NO: 4534. In some embodiments, the AAV particle comprises a viral genome comprising SEQ ID NO: 2216 and a capsid encoded by a nucleic acid sequence comprising SEQ ID NO: 3. In some embodiments, the AAV particle comprises a viral genome comprising SEQ ID NO: 2216 and a capsid comprising SEQ ID NO: 138. In some embodiments, the AAV particle comprises a viral genome comprising SEQ ID NO: 2216 and a capsid encoded by a nucleic acid sequence comprising SEQ ID NO: 137. In some embodiments, the AAV particle comprises a viral genome comprising SEQ ID NO: 2216 and a capsid comprising SEQ ID NO: 5. In some embodiments, the AAV particle comprises a viral genome comprising SEQ ID NO: 2216 and a capsid encoded by a nucleic acid sequence comprising SEQ ID NO: 6. In some embodiments, the AAV particle comprises a viral genome comprising SEQ ID NO: 2216 and a capsid comprising SEQ ID NO: 4. In some embodiments, the AAV particle comprises a viral genome comprising SEQ ID NO: 2216 and a capsid comprising SEQ ID NO: 11. In some embodiments, the AAV particle comprises a viral genome comprising SEQ ID NO: 2216 and a capsid comprising SEQ ID NO: 2679. In some embodiments, the AAV particle comprises a viral genome comprising SEQ ID NO: 2216 and a capsid comprising SEQ ID NO: 2809. In some embodiments, the AAV particle comprises a viral genome comprising SEQ ID NO: 2216 and a capsid comprising SEQ ID NO: 2871.

In some embodiments, the AAV particle comprises a viral genome comprising SEQ ID NO: 2217 and a VOY101 capsid. In some embodiments, the AAV particle comprises a viral genome comprising SEQ ID NO: 2217 and a VOY201 capsid. In some embodiments, the AAV particle comprises a viral genome comprising SEQ ID NO: 2217 and an AAV9 capsid. In some embodiments, the AAV particle comprises a viral genome comprising SEQ ID NO: 2217 and an AAV9K449R capsid. In some embodiments, the AAV particle comprises a viral genome comprising SEQ ID NO: 2217 and an AAVPHP.B capsid. In some embodiments, the AAV particle comprises a viral genome comprising SEQ ID NO: 2217 and an AAVPHP.N capsid. In some embodiments, the AAV particle comprises a viral genome comprising SEQ ID NO: 2217 and an AAV2 variant capsid. In some embodiments, the AAV particle comprises a viral genome comprising SEQ ID NO: 2217 and an AAV2/3 variant capsid. In some embodiments, the AAV particle comprises a viral genome comprising SEQ ID NO: 2217 and a capsid comprising SEQ ID NO: 1. In some embodiments, the AAV particle comprises a viral genome comprising SEQ ID NO: 2217 and a capsid encoded by a nucleic acid sequence comprising SEQ ID NO: 2. In some embodiments, the AAV particle comprises a viral genome comprising SEQ ID NO: 2217 and a capsid comprising SEQ ID NO: 4534. In some embodiments, the AAV particle comprises a viral genome comprising SEQ ID NO: 2217 and a capsid encoded by a nucleic acid sequence comprising SEQ ID NO: 3. In some embodiments, the AAV particle comprises a viral genome comprising SEQ ID NO: 2217 and a capsid comprising SEQ ID NO: 138. In some embodiments, the AAV particle comprises a viral genome comprising SEQ ID NO: 2217 and a capsid encoded by a nucleic acid sequence comprising SEQ ID NO: 137. In some embodiments, the AAV particle comprises a viral genome comprising SEQ ID NO: 2217 and a capsid comprising SEQ ID NO: 5. In some embodiments, the AAV particle comprises a viral genome comprising SEQ ID NO: 2217 and a capsid encoded by a nucleic acid sequence comprising SEQ ID NO: 6. In some embodiments, the AAV particle comprises a viral genome comprising SEQ ID NO: 2217 and a capsid comprising SEQ ID NO: 4. In some embodiments, the AAV particle comprises a viral genome comprising SEQ ID NO: 2217 and a capsid comprising SEQ ID NO: 11. In some embodiments, the AAV particle comprises a viral genome comprising SEQ ID NO: 2217 and a capsid comprising SEQ ID NO: 2679. In some embodiments, the AAV particle comprises a viral genome comprising SEQ ID NO: 2217 and a capsid comprising SEQ ID NO: 2809. In some embodiments, the AAV particle comprises a viral genome comprising SEQ ID NO: 2217 and a capsid comprising SEQ ID NO: 2871.

In some embodiments, the AAV particle comprises a viral genome comprising SEQ ID NO: 2218 and a VOY101 capsid. In some embodiments, the AAV particle comprises a viral genome comprising SEQ ID NO: 2218 and a VOY201 capsid. In some embodiments, the AAV particle comprises a viral genome comprising SEQ ID NO: 2218 and an AAV9 capsid. In some embodiments, the AAV particle comprises a viral genome comprising SEQ ID NO: 2218 and an AAV9K449R capsid. In some embodiments, the AAV particle comprises a viral genome comprising SEQ ID NO: 2218 and an AAVPHP.B capsid. In some embodiments, the AAV particle comprises a viral genome comprising SEQ ID NO: 2218 and an AAVPHP.N capsid. In some embodiments, the AAV particle comprises a viral genome comprising SEQ ID NO: 2218 and an AAV2 variant capsid. In some embodiments, the AAV particle comprises a viral genome comprising SEQ ID NO: 2218 and an AAV2/3 variant capsid. In some embodiments, the AAV particle comprises a viral genome comprising SEQ ID NO: 2218 and a capsid comprising SEQ ID NO: 1. In some embodiments, the AAV particle comprises a viral genome comprising SEQ ID NO: 2218 and a capsid encoded by a nucleic acid sequence comprising SEQ ID NO: 2. In some embodiments, the AAV particle comprises a viral genome comprising SEQ ID NO: 2218 and a capsid comprising SEQ ID NO: 4534. In some embodiments, the AAV particle comprises a viral genome comprising SEQ ID NO: 2218 and a capsid encoded by a nucleic acid sequence comprising SEQ ID NO: 3. In some embodiments, the AAV particle comprises a viral genome comprising SEQ ID NO: 2218 and a capsid comprising SEQ ID NO: 138. In some embodiments, the AAV particle comprises a viral genome comprising SEQ ID NO: 2218 and a capsid encoded by a nucleic acid sequence comprising SEQ ID NO: 137. In some embodiments, the AAV particle comprises a viral genome comprising SEQ ID NO: 2218 and a capsid comprising SEQ ID NO: 5. In some embodiments, the AAV particle comprises a viral genome comprising SEQ ID NO: 2218 and a capsid encoded by a nucleic acid sequence comprising SEQ ID NO: 6. In some embodiments, the AAV particle comprises a viral genome comprising SEQ ID NO: 2218 and a capsid comprising SEQ ID NO: 4. In some embodiments, the AAV particle comprises a viral genome comprising SEQ ID NO: 2218 and a capsid comprising SEQ ID NO: 11. In some embodiments, the AAV particle comprises a viral genome comprising SEQ ID NO: 2218 and a capsid comprising SEQ ID NO: 2679. In some embodiments, the AAV particle comprises a viral genome comprising SEQ ID NO: 2218 and a capsid comprising SEQ ID NO: 2809. In some embodiments, the AAV particle comprises a viral genome comprising SEQ ID NO: 2218 and a capsid comprising SEQ ID NO: 2871.

In some embodiments, the AAV particle comprises a viral genome comprising SEQ ID NO: 2219 and a VOY101 capsid. In some embodiments, the AAV particle comprises a viral genome comprising SEQ ID NO: 2219 and a VOY201 capsid. In some embodiments, the AAV particle comprises a viral genome comprising SEQ ID NO: 2219 and an AAV9 capsid. In some embodiments, the AAV particle comprises a viral genome comprising SEQ ID NO: 2219 and an AAV9K449R capsid. In some embodiments, the AAV particle comprises a viral genome comprising SEQ ID NO: 2219 and an AAVPHP.B capsid. In some embodiments, the AAV particle comprises a viral genome comprising SEQ ID NO: 2219 and an AAVPHP.N capsid. In some embodiments, the AAV particle comprises a viral genome comprising SEQ ID NO: 2219 and an AAV2 variant capsid. In some embodiments, the AAV particle comprises a viral genome comprising SEQ ID NO: 2219 and an AAV2/3 variant capsid. In some embodiments, the AAV particle comprises a viral genome comprising SEQ ID NO: 2219 and a capsid comprising SEQ ID NO: 1. In some embodiments, the AAV particle comprises a viral genome comprising SEQ ID NO: 2219 and a capsid encoded by a nucleic acid sequence comprising SEQ ID NO: 2. In some embodiments, the AAV particle comprises a viral genome comprising SEQ ID NO: 2219 and a capsid comprising SEQ ID NO: 4534. In some embodiments, the AAV particle comprises a viral genome comprising SEQ ID NO: 2219 and a capsid encoded by a nucleic acid sequence comprising SEQ ID NO: 3. In some embodiments, the AAV particle comprises a viral genome comprising SEQ ID NO: 2219 and a capsid comprising SEQ ID NO: 138. In some embodiments, the AAV particle comprises a viral genome comprising SEQ ID NO: 2219 and a capsid encoded by a nucleic acid sequence comprising SEQ ID NO: 137. In some embodiments, the AAV particle comprises a viral genome comprising SEQ ID NO: 2219 and a capsid comprising SEQ ID NO: 5. In some embodiments, the AAV particle comprises a viral genome comprising SEQ ID NO: 2219 and a capsid encoded by a nucleic acid sequence comprising SEQ ID NO: 6. In some embodiments, the AAV particle comprises a viral genome comprising SEQ ID NO: 2219 and a capsid comprising SEQ ID NO: 4. In some embodiments, the AAV particle comprises a viral genome comprising SEQ ID NO: 2219 and a capsid comprising SEQ ID NO: 11. In some embodiments, the AAV particle comprises a viral genome comprising SEQ ID NO: 2219 and a capsid comprising SEQ ID NO: 2679. In some embodiments, the AAV particle comprises a viral genome comprising SEQ ID NO: 2219 and a capsid comprising SEQ ID NO: 2809. In some embodiments, the AAV particle comprises a viral genome comprising SEQ ID NO: 2219 and a capsid comprising SEQ ID NO: 2871.

In some embodiments, the AAV particle comprises a viral genome comprising SEQ ID NO: 2220 and a VOY101 capsid. In some embodiments, the AAV particle comprises a viral genome comprising SEQ ID NO: 2220 and a VOY201 capsid. In some embodiments, the AAV particle comprises a viral genome comprising SEQ ID NO: 2220 and an AAV9 capsid. In some embodiments, the AAV particle comprises a viral genome comprising SEQ ID NO: 2220 and an AAV9K449R capsid. In some embodiments, the AAV particle comprises a viral genome comprising SEQ ID NO: 2220 and an AAVPHP.B capsid. In some embodiments, the AAV particle comprises a viral genome comprising SEQ ID NO: 2220 and an AAVPHP.N capsid. In some embodiments, the AAV particle comprises a viral genome comprising SEQ ID NO: 2220 and an AAV2 variant capsid. In some embodiments, the AAV particle comprises a viral genome comprising SEQ ID NO: 2220 and an AAV2/3 variant capsid. In some embodiments, the AAV particle comprises a viral genome comprising SEQ ID NO: 2220 and a capsid comprising SEQ ID NO: 1. In some embodiments, the AAV particle comprises a viral genome comprising SEQ ID NO: 2220 and a capsid encoded by a nucleic acid sequence comprising SEQ ID NO: 2. In some embodiments, the AAV particle comprises a viral genome comprising SEQ ID NO: 2220 and a capsid comprising SEQ ID NO: 4534. In some embodiments, the AAV particle comprises a viral genome comprising SEQ ID NO: 2220 and a capsid encoded by a nucleic acid sequence comprising SEQ ID NO: 3. In some embodiments, the AAV particle comprises a viral genome comprising SEQ ID NO: 2220 and a capsid comprising SEQ ID NO: 138. In some embodiments, the AAV particle comprises a viral genome comprising SEQ ID NO: 2220 and a capsid encoded by a nucleic acid sequence comprising SEQ ID NO: 137. In some embodiments, the AAV particle comprises a viral genome comprising SEQ ID NO: 2220 and a capsid comprising SEQ ID NO: 5. In some embodiments, the AAV particle comprises a viral genome comprising SEQ ID NO: 2220 and a capsid encoded by a nucleic acid sequence comprising SEQ ID NO: 6. In some embodiments, the AAV particle comprises a viral genome comprising SEQ ID NO: 2220 and a capsid comprising SEQ ID NO: 4. In some embodiments, the AAV particle comprises a viral genome comprising SEQ ID NO: 2220 and a capsid comprising SEQ ID NO: 11. In some embodiments, the AAV particle comprises a viral genome comprising SEQ ID NO: 2220 and a capsid comprising SEQ ID NO: 2679. In some embodiments, the AAV particle comprises a viral genome comprising SEQ ID NO: 2220 and a capsid comprising SEQ ID NO: 2809. In some embodiments, the AAV particle comprises a viral genome comprising SEQ ID NO: 2220 and a capsid comprising SEQ ID NO: 2871.

In some embodiments, the AAV particle comprises a viral genome comprising SEQ ID NO: 2221 and a VOY101 capsid. In some embodiments, the AAV particle comprises a viral genome comprising SEQ ID NO: 2221 and a VOY201 capsid. In some embodiments, the AAV particle comprises a viral genome comprising SEQ ID NO: 2221 and an AAV9 capsid. In some embodiments, the AAV particle comprises a viral genome comprising SEQ ID NO: 2221 and an AAV9K449R capsid. In some embodiments, the AAV particle comprises a viral genome comprising SEQ ID NO: 2221 and an AAVPHP.B capsid. In some embodiments, the AAV particle comprises a viral genome comprising SEQ ID NO: 2221 and an AAVPHP.N capsid. In some embodiments, the AAV particle comprises a viral genome comprising SEQ ID NO: 2221 and an AAV2 variant capsid. In some embodiments, the AAV particle comprises a viral genome comprising SEQ ID NO: 2221 and an AAV2/3 variant capsid. In some embodiments, the AAV particle comprises a viral genome comprising SEQ ID NO: 2221 and a capsid comprising SEQ ID NO: 1. In some embodiments, the AAV particle comprises a viral genome comprising SEQ ID NO: 2221 and a capsid encoded by a nucleic acid sequence comprising SEQ ID NO: 2. In some embodiments, the AAV particle comprises a viral genome comprising SEQ ID NO: 2221 and a capsid comprising SEQ ID NO: 4534. In some embodiments, the AAV particle comprises a viral genome comprising SEQ ID NO: 2221 and a capsid encoded by a nucleic acid sequence comprising SEQ ID NO: 3. In some embodiments, the AAV particle comprises a viral genome comprising SEQ ID NO: 2221 and a capsid comprising SEQ ID NO: 138. In some embodiments, the AAV particle comprises a viral genome comprising SEQ ID NO: 2221 and a capsid encoded by a nucleic acid sequence comprising SEQ ID NO: 137. In some embodiments, the AAV particle comprises a viral genome comprising SEQ ID NO: 2221 and a capsid comprising SEQ ID NO: 5. In some embodiments, the AAV particle comprises a viral genome comprising SEQ ID NO: 2221 and a capsid encoded by a nucleic acid sequence comprising SEQ ID NO: 6. In some embodiments, the AAV particle comprises a viral genome comprising SEQ ID NO: 2221 and a capsid comprising SEQ ID NO: 4. In some embodiments, the AAV particle comprises a viral genome comprising SEQ ID NO: 2221 and a capsid comprising SEQ ID NO: 11. In some embodiments, the AAV particle comprises a viral genome comprising SEQ ID NO: 2221 and a capsid comprising SEQ ID NO: 2679. In some embodiments, the AAV particle comprises a viral genome comprising SEQ ID NO: 2221 and a capsid comprising SEQ ID NO: 2809. In some embodiments, the AAV particle comprises a viral genome comprising SEQ ID NO: 2221 and a capsid comprising SEQ ID NO: 2871.

In some embodiments, the AAV particle comprises a viral genome comprising SEQ ID NO: 2222 and a VOY101 capsid. In some embodiments, the AAV particle comprises a viral genome comprising SEQ ID NO: 2222 and a VOY201 capsid. In some embodiments, the AAV particle comprises a viral genome comprising SEQ ID NO: 2222 and an AAV9 capsid. In some embodiments, the AAV particle comprises a viral genome comprising SEQ ID NO: 2222 and an AAV9K449R capsid. In some embodiments, the AAV particle comprises a viral genome comprising SEQ ID NO: 2222 and an AAVPHP.B capsid. In some embodiments, the AAV particle comprises a viral genome comprising SEQ ID NO: 2222 and an AAVPHP.N capsid. In some embodiments, the AAV particle comprises a viral genome comprising SEQ ID NO: 2222 and an AAV2 variant capsid. In some embodiments, the AAV particle comprises a viral genome comprising SEQ ID NO: 2222 and an AAV2/3 variant capsid. In some embodiments, the AAV particle comprises a viral genome comprising SEQ ID NO: 2222 and a capsid comprising SEQ ID NO: 1. In some embodiments, the AAV particle comprises a viral genome comprising SEQ ID NO: 2222 and a capsid encoded by a nucleic acid sequence comprising SEQ ID NO: 2. In some embodiments, the AAV particle comprises a viral genome comprising SEQ ID NO: 2222 and a capsid comprising SEQ ID NO: 4534. In some embodiments, the AAV particle comprises a viral genome comprising SEQ ID NO: 2222 and a capsid encoded by a nucleic acid sequence comprising SEQ ID NO: 3. In some embodiments, the AAV particle comprises a viral genome comprising SEQ ID NO: 2222 and a capsid comprising SEQ ID NO: 138. In some embodiments, the AAV particle comprises a viral genome comprising SEQ ID NO: 2222 and a capsid encoded by a nucleic acid sequence comprising SEQ ID NO: 137. In some embodiments, the AAV particle comprises a viral genome comprising SEQ ID NO: 2222 and a capsid comprising SEQ ID NO: 5. In some embodiments, the AAV particle comprises a viral genome comprising SEQ ID NO: 2222 and a capsid encoded by a nucleic acid sequence comprising SEQ ID NO: 6. In some embodiments, the AAV particle comprises a viral genome comprising SEQ ID NO: 2222 and a capsid comprising SEQ ID NO: 4. In some embodiments, the AAV particle comprises a viral genome comprising SEQ ID NO: 2222 and a capsid comprising SEQ ID NO: 11. In some embodiments, the AAV particle comprises a viral genome comprising SEQ ID NO: 2222 and a capsid comprising SEQ ID NO: 2679. In some embodiments, the AAV particle comprises a viral genome comprising SEQ ID NO: 2222 and a capsid comprising SEQ ID NO: 2809. In some embodiments, the AAV particle comprises a viral genome comprising SEQ ID NO: 2222 and a capsid comprising SEQ ID NO: 2871.

In some embodiments, the AAV particle comprises a viral genome comprising SEQ ID NO: 2223 and a VOY101 capsid. In some embodiments, the AAV particle comprises a viral genome comprising SEQ ID NO: 2223 and a VOY201 capsid. In some embodiments, the AAV particle comprises a viral genome comprising SEQ ID NO: 2223 and an AAV9 capsid. In some embodiments, the AAV particle comprises a viral genome comprising SEQ ID NO: 2223 and an AAV9K449R capsid. In some embodiments, the AAV particle comprises a viral genome comprising SEQ ID NO: 2223 and an AAVPHP.B capsid. In some embodiments, the AAV particle comprises a viral genome comprising SEQ ID NO: 2223 and an AAVPHP.N capsid. In some embodiments, the AAV particle comprises a viral genome comprising SEQ ID NO: 2223 and an AAV2 variant capsid. In some embodiments, the AAV particle comprises a viral genome comprising SEQ ID NO: 2223 and an AAV2/3 variant capsid. In some embodiments, the AAV particle comprises a viral genome comprising SEQ ID NO: 2223 and a capsid comprising SEQ ID NO: 1. In some embodiments, the AAV particle comprises a viral genome comprising SEQ ID NO: 2223 and a capsid encoded by a nucleic acid sequence comprising SEQ ID NO: 2. In some embodiments, the AAV particle comprises a viral genome comprising SEQ ID NO: 2223 and a capsid comprising SEQ ID NO: 4534. In some embodiments, the AAV particle comprises a viral genome comprising SEQ ID NO: 2223 and a capsid encoded by a nucleic acid sequence comprising SEQ ID NO: 3. In some embodiments, the AAV particle comprises a viral genome comprising SEQ ID NO: 2223 and a capsid comprising SEQ ID NO: 138. In some embodiments, the AAV particle comprises a viral genome comprising SEQ ID NO: 2223 and a capsid encoded by a nucleic acid sequence comprising SEQ ID NO: 137. In some embodiments, the AAV particle comprises a viral genome comprising SEQ ID NO: 2223 and a capsid comprising SEQ ID NO: 5. In some embodiments, the AAV particle comprises a viral genome comprising SEQ ID NO: 2223 and a capsid encoded by a nucleic acid sequence comprising SEQ ID NO: 6. In some embodiments, the AAV particle comprises a viral genome comprising SEQ ID NO: 2223 and a capsid comprising SEQ ID NO: 4. In some embodiments, the AAV particle comprises a viral genome comprising SEQ ID NO: 2223 and a capsid comprising SEQ ID NO: 11. In some embodiments, the AAV particle comprises a viral genome comprising SEQ ID NO: 2223 and a capsid comprising SEQ ID NO: 2679. In some embodiments, the AAV particle comprises a viral genome comprising SEQ ID NO: 2223 and a capsid comprising SEQ ID NO: 2809. In some embodiments, the AAV particle comprises a viral genome comprising SEQ ID NO: 2223 and a capsid comprising SEQ ID NO: 2871.

In some embodiments, the AAV particle comprises a viral genome comprising SEQ ID NO: 2224 and a VOY101 capsid. In some embodiments, the AAV particle comprises a viral genome comprising SEQ ID NO: 2224 and a VOY201 capsid. In some embodiments, the AAV particle comprises a viral genome comprising SEQ ID NO: 2224 and an AAV9 capsid. In some embodiments, the AAV particle comprises a viral genome comprising SEQ ID NO: 2224 and an AAV9K449R capsid. In some embodiments, the AAV particle comprises a viral genome comprising SEQ ID NO: 2224 and an AAVPHP.B capsid. In some embodiments, the AAV particle comprises a viral genome comprising SEQ ID NO: 2224 and an AAVPHP.N capsid. In some embodiments, the AAV particle comprises a viral genome comprising SEQ ID NO: 2224 and an AAV2 variant capsid. In some embodiments, the AAV particle comprises a viral genome comprising SEQ ID NO: 2224 and an AAV2/3 variant capsid. In some embodiments, the AAV particle comprises a viral genome comprising SEQ ID NO: 2224 and a capsid comprising SEQ ID NO: 1. In some embodiments, the AAV particle comprises a viral genome comprising SEQ ID NO: 2224 and a capsid encoded by a nucleic acid sequence comprising SEQ ID NO: 2. In some embodiments, the AAV particle comprises a viral genome comprising SEQ ID NO: 2224 and a capsid comprising SEQ ID NO: 4534. In some embodiments, the AAV particle comprises a viral genome comprising SEQ ID NO: 2224 and a capsid encoded by a nucleic acid sequence comprising SEQ ID NO: 3. In some embodiments, the AAV particle comprises a viral genome comprising SEQ ID NO: 2224 and a capsid comprising SEQ ID NO: 138. In some embodiments, the AAV particle comprises a viral genome comprising SEQ ID NO: 2224 and a capsid encoded by a nucleic acid sequence comprising SEQ ID NO: 137. In some embodiments, the AAV particle comprises a viral genome comprising SEQ ID NO: 2224 and a capsid comprising SEQ ID NO: 5. In some embodiments, the AAV particle comprises a viral genome comprising SEQ ID NO: 2224 and a capsid encoded by a nucleic acid sequence comprising SEQ ID NO: 6. In some embodiments, the AAV particle comprises a viral genome comprising SEQ ID NO: 2224 and a capsid comprising SEQ ID NO: 4. In some embodiments, the AAV particle comprises a viral genome comprising SEQ ID NO: 2224 and a capsid comprising SEQ ID NO: 11. In some embodiments, the AAV particle comprises a viral genome comprising SEQ ID NO: 2224 and a capsid comprising SEQ ID NO: 2679. In some embodiments, the AAV particle comprises a viral genome comprising SEQ ID NO: 2224 and a capsid comprising SEQ ID NO: 2809. In some embodiments, the AAV particle comprises a viral genome comprising SEQ ID NO: 2224 and a capsid comprising SEQ ID NO: 2871.

In some embodiments, the AAV particle comprises a viral genome comprising SEQ ID NO: 2225 and a VOY101 capsid. In some embodiments, the AAV particle comprises a viral genome comprising SEQ ID NO: 2225 and a VOY201 capsid. In some embodiments, the AAV particle comprises a viral genome comprising SEQ ID NO: 2225 and an AAV9 capsid. In some embodiments, the AAV particle comprises a viral genome comprising SEQ ID NO: 2225 and an AAV9K449R capsid. In some embodiments, the AAV particle comprises a viral genome comprising SEQ ID NO: 2225 and an AAVPHP.B capsid. In some embodiments, the AAV particle comprises a viral genome comprising SEQ ID NO: 2225 and an AAVPHP.N capsid. In some embodiments, the AAV particle comprises a viral genome comprising SEQ ID NO: 2225 and an AAV2 variant capsid. In some embodiments, the AAV particle comprises a viral genome comprising SEQ ID NO: 2225 and an AAV2/3 variant capsid. In some embodiments, the AAV particle comprises a viral genome comprising SEQ ID NO: 2225 and a capsid comprising SEQ ID NO: 1. In some embodiments, the AAV particle comprises a viral genome comprising SEQ ID NO: 2225 and a capsid encoded by a nucleic acid sequence comprising SEQ ID NO: 2. In some embodiments, the AAV particle comprises a viral genome comprising SEQ ID NO: 2225 and a capsid comprising SEQ ID NO: 4534. In some embodiments, the AAV particle comprises a viral genome comprising SEQ ID NO: 2225 and a capsid encoded by a nucleic acid sequence comprising SEQ ID NO: 3. In some embodiments, the AAV particle comprises a viral genome comprising SEQ ID NO: 2225 and a capsid comprising SEQ ID NO: 138. In some embodiments, the AAV particle comprises a viral genome comprising SEQ ID NO: 2225 and a capsid encoded by a nucleic acid sequence comprising SEQ ID NO: 137. In some embodiments, the AAV particle comprises a viral genome comprising SEQ ID NO: 2225 and a capsid comprising SEQ ID NO: 5. In some embodiments, the AAV particle comprises a viral genome comprising SEQ ID NO: 2225 and a capsid encoded by a nucleic acid sequence comprising SEQ ID NO: 6. In some embodiments, the AAV particle comprises a viral genome comprising SEQ ID NO: 2225 and a capsid comprising SEQ ID NO: 4. In some embodiments, the AAV particle comprises a viral genome comprising SEQ ID NO: 2225 and a capsid comprising SEQ ID NO: 11. In some embodiments, the AAV particle comprises a viral genome comprising SEQ ID NO: 2225 and a capsid comprising SEQ ID NO: 2679. In some embodiments, the AAV particle comprises a viral genome comprising SEQ ID NO: 2225 and a capsid comprising SEQ ID NO: 2809. In some embodiments, the AAV particle comprises a viral genome comprising SEQ ID NO: 2225 and a capsid comprising SEQ ID NO: 2871.

In some embodiments, the AAV particle comprises a viral genome comprising SEQ ID NO: 2226 and a VOY101 capsid. In some embodiments, the AAV particle comprises a viral genome comprising SEQ ID NO: 2226 and a VOY201 capsid. In some embodiments, the AAV particle comprises a viral genome comprising SEQ ID NO: 2226 and an AAV9 capsid. In some embodiments, the AAV particle comprises a viral genome comprising SEQ ID NO: 2226 and an AAV9K449R capsid. In some embodiments, the AAV particle comprises a viral genome comprising SEQ ID NO: 2226 and an AAVPHP.B capsid. In some embodiments, the AAV particle comprises a viral genome comprising SEQ ID NO: 2226 and an AAVPHP.N capsid. In some embodiments, the AAV particle comprises a viral genome comprising SEQ ID NO: 2226 and an AAV2 variant capsid. In some embodiments, the AAV particle comprises a viral genome comprising SEQ ID NO: 2226 and an AAV2/3 variant capsid. In some embodiments, the AAV particle comprises a viral genome comprising SEQ ID NO: 2226 and a capsid comprising SEQ ID NO: 1. In some embodiments, the AAV particle comprises a viral genome comprising SEQ ID NO: 2226 and a capsid encoded by a nucleic acid sequence comprising SEQ ID NO: 2. In some embodiments, the AAV particle comprises a viral genome comprising SEQ ID NO: 2226 and a capsid comprising SEQ ID NO: 4534. In some embodiments, the AAV particle comprises a viral genome comprising SEQ ID NO: 2226 and a capsid encoded by a nucleic acid sequence comprising SEQ ID NO: 3. In some embodiments, the AAV particle comprises a viral genome comprising SEQ ID NO: 2226 and a capsid comprising SEQ ID NO: 138. In some embodiments, the AAV particle comprises a viral genome comprising SEQ ID NO: 2226 and a capsid encoded by a nucleic acid sequence comprising SEQ ID NO: 137. In some embodiments, the AAV particle comprises a viral genome comprising SEQ ID NO: 2226 and a capsid comprising SEQ ID NO: 5. In some embodiments, the AAV particle comprises a viral genome comprising SEQ ID NO: 2226 and a capsid encoded by a nucleic acid sequence comprising SEQ ID NO: 6. In some embodiments, the AAV particle comprises a viral genome comprising SEQ ID NO: 2226 and a capsid comprising SEQ ID NO: 4. In some embodiments, the AAV particle comprises a viral genome comprising SEQ ID NO: 2226 and a capsid comprising SEQ ID NO: 11. In some embodiments, the AAV particle comprises a viral genome comprising SEQ ID NO: 2226 and a capsid comprising SEQ ID NO: 2679. In some embodiments, the AAV particle comprises a viral genome comprising SEQ ID NO: 2226 and a capsid comprising SEQ ID NO: 2809. In some embodiments, the AAV particle comprises a viral genome comprising SEQ ID NO: 2226 and a capsid comprising SEQ ID NO: 2871.

In some embodiments, the AAV particle comprises a viral genome comprising SEQ ID NO: 2227 and a VOY101 capsid. In some embodiments, the AAV particle comprises a viral genome comprising SEQ ID NO: 2227 and a VOY201 capsid. In some embodiments, the AAV particle comprises a viral genome comprising SEQ ID NO: 2227 and an AAV9 capsid. In some embodiments, the AAV particle comprises a viral genome comprising SEQ ID NO: 2227 and an AAV9K449R capsid. In some embodiments, the AAV particle comprises a viral genome comprising SEQ ID NO: 2227 and an AAVPHP.B capsid. In some embodiments, the AAV particle comprises a viral genome comprising SEQ ID NO: 2227 and an AAVPHP.N capsid. In some embodiments, the AAV particle comprises a viral genome comprising SEQ ID NO: 2227 and an AAV2 variant capsid. In some embodiments, the AAV particle comprises a viral genome comprising SEQ ID NO: 2227 and an AAV2/3 variant capsid. In some embodiments, the AAV particle comprises a viral genome comprising SEQ ID NO: 2227 and a capsid comprising SEQ ID NO: 1. In some embodiments, the AAV particle comprises a viral genome comprising SEQ ID NO: 2227 and a capsid encoded by a nucleic acid sequence comprising SEQ ID NO: 2. In some embodiments, the AAV particle comprises a viral genome comprising SEQ ID NO: 2227 and a capsid comprising SEQ ID NO: 4534. In some embodiments, the AAV particle comprises a viral genome comprising SEQ ID NO: 2227 and a capsid encoded by a nucleic acid sequence comprising SEQ ID NO: 3. In some embodiments, the AAV particle comprises a viral genome comprising SEQ ID NO: 2227 and a capsid comprising SEQ ID NO: 138. In some embodiments, the AAV particle comprises a viral genome comprising SEQ ID NO: 2227 and a capsid encoded by a nucleic acid sequence comprising SEQ ID NO: 137. In some embodiments, the AAV particle comprises a viral genome comprising SEQ ID NO: 2227 and a capsid comprising SEQ ID NO: 5. In some embodiments, the AAV particle comprises a viral genome comprising SEQ ID NO: 2227 and a capsid encoded by a nucleic acid sequence comprising SEQ ID NO: 6. In some embodiments, the AAV particle comprises a viral genome comprising SEQ ID NO: 2227 and a capsid comprising SEQ ID NO: 4. In some embodiments, the AAV particle comprises a viral genome comprising SEQ ID NO: 2227 and a capsid comprising SEQ ID NO: 11. In some embodiments, the AAV particle comprises a viral genome comprising SEQ ID NO: 2227 and a capsid comprising SEQ ID NO: 2679. In some embodiments, the AAV particle comprises a viral genome comprising SEQ ID NO: 2227 and a capsid comprising SEQ ID NO: 2809. In some embodiments, the AAV particle comprises a viral genome comprising SEQ ID NO: 2227 and a capsid comprising SEQ ID NO: 2871.

In some embodiments, the AAV particle comprises a viral genome comprising SEQ ID NO: 2228 and a VOY101 capsid. In some embodiments, the AAV particle comprises a viral genome comprising SEQ ID NO: 2228 and a VOY201 capsid. In some embodiments, the AAV particle comprises a viral genome comprising SEQ ID NO: 2228 and an AAV9 capsid. In some embodiments, the AAV particle comprises a viral genome comprising SEQ ID NO: 2228 and an AAV9K449R capsid. In some embodiments, the AAV particle comprises a viral genome comprising SEQ ID NO: 2228 and an AAVPHP.B capsid. In some embodiments, the AAV particle comprises a viral genome comprising SEQ ID NO: 2228 and an AAVPHP.N capsid. In some embodiments, the AAV particle comprises a viral genome comprising SEQ ID NO: 2228 and an AAV2 variant capsid. In some embodiments, the AAV particle comprises a viral genome comprising SEQ ID NO: 2228 and an AAV2/3 variant capsid. In some embodiments, the AAV particle comprises a viral genome comprising SEQ ID NO: 2228 and a capsid comprising SEQ ID NO: 1. In some embodiments, the AAV particle comprises a viral genome comprising SEQ ID NO: 2228 and a capsid encoded by a nucleic acid sequence comprising SEQ ID NO: 2. In some embodiments, the AAV particle comprises a viral genome comprising SEQ ID NO: 2228 and a capsid comprising SEQ ID NO: 4534. In some embodiments, the AAV particle comprises a viral genome comprising SEQ ID NO: 2228 and a capsid encoded by a nucleic acid sequence comprising SEQ ID NO: 3. In some embodiments, the AAV particle comprises a viral genome comprising SEQ ID NO: 2228 and a capsid comprising SEQ ID NO: 138. In some embodiments, the AAV particle comprises a viral genome comprising SEQ ID NO: 2228 and a capsid encoded by a nucleic acid sequence comprising SEQ ID NO: 137. In some embodiments, the AAV particle comprises a viral genome comprising SEQ ID NO: 2228 and a capsid comprising SEQ ID NO: 5. In some embodiments, the AAV particle comprises a viral genome comprising SEQ ID NO: 2228 and a capsid encoded by a nucleic acid sequence comprising SEQ ID NO: 6. In some embodiments, the AAV particle comprises a viral genome comprising SEQ ID NO: 2228 and a capsid comprising SEQ ID NO: 4. In some embodiments, the AAV particle comprises a viral genome comprising SEQ ID NO: 2228 and a capsid comprising SEQ ID NO: 11. In some embodiments, the AAV particle comprises a viral genome comprising SEQ ID NO: 2228 and a capsid comprising SEQ ID NO: 2679. In some embodiments, the AAV particle comprises a viral genome comprising SEQ ID NO: 2228 and a capsid comprising SEQ ID NO: 2809. In some embodiments, the AAV particle comprises a viral genome comprising SEQ ID NO: 2228 and a capsid comprising SEQ ID NO: 2871.

In some embodiments, the AAV particle comprises a viral genome comprising SEQ ID NO: 2229 and a VOY101 capsid. In some embodiments, the AAV particle comprises a viral genome comprising SEQ ID NO: 2229 and a VOY201 capsid. In some embodiments, the AAV particle comprises a viral genome comprising SEQ ID NO: 2229 and an AAV9 capsid. In some embodiments, the AAV particle comprises a viral genome comprising SEQ ID NO: 2229 and an AAV9K449R capsid. In some embodiments, the AAV particle comprises a viral genome comprising SEQ ID NO: 2229 and an AAVPHP.B capsid. In some embodiments, the AAV particle comprises a viral genome comprising SEQ ID NO: 2229 and an AAVPHP.N capsid. In some embodiments, the AAV particle comprises a viral genome comprising SEQ ID NO: 2229 and an AAV2 variant capsid. In some embodiments, the AAV particle comprises a viral genome comprising SEQ ID NO: 2229 and an AAV2/3 variant capsid. In some embodiments, the AAV particle comprises a viral genome comprising SEQ ID NO: 2229 and a capsid comprising SEQ ID NO: 1. In some embodiments, the AAV particle comprises a viral genome comprising SEQ ID NO: 2229 and a capsid encoded by a nucleic acid sequence comprising SEQ ID NO: 2. In some embodiments, the AAV particle comprises a viral genome comprising SEQ ID NO: 2229 and a capsid comprising SEQ ID NO: 4534. In some embodiments, the AAV particle comprises a viral genome comprising SEQ ID NO: 2229 and a capsid encoded by a nucleic acid sequence comprising SEQ ID NO: 3. In some embodiments, the AAV particle comprises a viral genome comprising SEQ ID NO: 2229 and a capsid comprising SEQ ID NO: 138. In some embodiments, the AAV particle comprises a viral genome comprising SEQ ID NO: 2229 and a capsid encoded by a nucleic acid sequence comprising SEQ ID NO: 137. In some embodiments, the AAV particle comprises a viral genome comprising SEQ ID NO: 2229 and a capsid comprising SEQ ID NO: 5. In some embodiments, the AAV particle comprises a viral genome comprising SEQ ID NO: 2229 and a capsid encoded by a nucleic acid sequence comprising SEQ ID NO: 6. In some embodiments, the AAV particle comprises a viral genome comprising SEQ ID NO: 2229 and a capsid comprising SEQ ID NO: 4. In some embodiments, the AAV particle comprises a viral genome comprising SEQ ID NO: 2229 and a capsid comprising SEQ ID NO: 11. In some embodiments, the AAV particle comprises a viral genome comprising SEQ ID NO: 2229 and a capsid comprising SEQ ID NO: 2679. In some embodiments, the AAV particle comprises a viral genome comprising SEQ ID NO: 2229 and a capsid comprising SEQ ID NO: 2809. In some embodiments, the AAV particle comprises a viral genome comprising SEQ ID NO: 2229 and a capsid comprising SEQ ID NO: 2871.

In some embodiments, the AAV particle comprises a viral genome comprising SEQ ID NO: 2230 and a VOY101 capsid. In some embodiments, the AAV particle comprises a viral genome comprising SEQ ID NO: 2230 and a VOY201 capsid. In some embodiments, the AAV particle comprises a viral genome comprising SEQ ID NO: 2230 and an AAV9 capsid. In some embodiments, the AAV particle comprises a viral genome comprising SEQ ID NO: 2230 and an AAV9K449R capsid. In some embodiments, the AAV particle comprises a viral genome comprising SEQ ID NO: 2230 and an AAVPHP.B capsid. In some embodiments, the AAV particle comprises a viral genome comprising SEQ ID NO: 2230 and an AAVPHP.N capsid. In some embodiments, the AAV particle comprises a viral genome comprising SEQ ID NO: 2230 and an AAV2 variant capsid. In some embodiments, the AAV particle comprises a viral genome comprising SEQ ID NO: 2230 and an AAV2/3 variant capsid. In some embodiments, the AAV particle comprises a viral genome comprising SEQ ID NO: 2230 and a capsid comprising SEQ ID NO: 1. In some embodiments, the AAV particle comprises a viral genome comprising SEQ ID NO: 2230 and a capsid encoded by a nucleic acid sequence comprising SEQ ID NO: 2. In some embodiments, the AAV particle comprises a viral genome comprising SEQ ID NO: 2230 and a capsid comprising SEQ ID NO: 4534. In some embodiments, the AAV particle comprises a viral genome comprising SEQ ID NO: 2230 and a capsid encoded by a nucleic acid sequence comprising SEQ ID NO: 3. In some embodiments, the AAV particle comprises a viral genome comprising SEQ ID NO: 2230 and a capsid comprising SEQ ID NO: 138. In some embodiments, the AAV particle comprises a viral genome comprising SEQ ID NO: 2230 and a capsid encoded by a nucleic acid sequence comprising SEQ ID NO: 137. In some embodiments, the AAV particle comprises a viral genome comprising SEQ ID NO: 2230 and a capsid comprising SEQ ID NO: 5. In some embodiments, the AAV particle comprises a viral genome comprising SEQ ID NO: 2230 and a capsid encoded by a nucleic acid sequence comprising SEQ ID NO: 6. In some embodiments, the AAV particle comprises a viral genome comprising SEQ ID NO: 2230 and a capsid comprising SEQ ID NO: 4. In some embodiments, the AAV particle comprises a viral genome comprising SEQ ID NO: 2230 and a capsid comprising SEQ ID NO: 11. In some embodiments, the AAV particle comprises a viral genome comprising SEQ ID NO: 2230 and a capsid comprising SEQ ID NO: 2679. In some embodiments, the AAV particle comprises a viral genome comprising SEQ ID NO: 2230 and a capsid comprising SEQ ID NO: 2809. In some embodiments, the AAV particle comprises a viral genome comprising SEQ ID NO: 2230 and a capsid comprising SEQ ID NO: 2871.

In some embodiments, the AAV particle comprises a viral genome comprising SEQ ID NO: 2231 and a VOY101 capsid. In some embodiments, the AAV particle comprises a viral genome comprising SEQ ID NO: 2231 and a VOY201 capsid. In some embodiments, the AAV particle comprises a viral genome comprising SEQ ID NO: 2231 and an AAV9 capsid. In some embodiments, the AAV particle comprises a viral genome comprising SEQ ID NO: 2231 and an AAV9K449R capsid. In some embodiments, the AAV particle comprises a viral genome comprising SEQ ID NO: 2231 and an AAVPHP.B capsid. In some embodiments, the AAV particle comprises a viral genome comprising SEQ ID NO: 2231 and an AAVPHP.N capsid. In some embodiments, the AAV particle comprises a viral genome comprising SEQ ID NO: 2231 and an AAV2 variant capsid. In some embodiments, the AAV particle comprises a viral genome comprising SEQ ID NO: 2231 and an AAV2/3 variant capsid. In some embodiments, the AAV particle comprises a viral genome comprising SEQ ID NO: 2231 and a capsid comprising SEQ ID NO: 1. In some embodiments, the AAV particle comprises a viral genome comprising SEQ ID NO: 2231 and a capsid encoded by a nucleic acid sequence comprising SEQ ID NO: 2. In some embodiments, the AAV particle comprises a viral genome comprising SEQ ID NO: 2231 and a capsid comprising SEQ ID NO: 4534. In some embodiments, the AAV particle comprises a viral genome comprising SEQ ID NO: 2231 and a capsid encoded by a nucleic acid sequence comprising SEQ ID NO: 3. In some embodiments, the AAV particle comprises a viral genome comprising SEQ ID NO: 2231 and a capsid comprising SEQ ID NO: 138. In some embodiments, the AAV particle comprises a viral genome comprising SEQ ID NO: 2231 and a capsid encoded by a nucleic acid sequence comprising SEQ ID NO: 137. In some embodiments, the AAV particle comprises a viral genome comprising SEQ ID NO: 2231 and a capsid comprising SEQ ID NO: 5. In some embodiments, the AAV particle comprises a viral genome comprising SEQ ID NO: 2231 and a capsid encoded by a nucleic acid sequence comprising SEQ ID NO: 6. In some embodiments, the AAV particle comprises a viral genome comprising SEQ ID NO: 2231 and a capsid comprising SEQ ID NO: 4. In some embodiments, the AAV particle comprises a viral genome comprising SEQ ID NO: 2231 and a capsid comprising SEQ ID NO: 11. In some embodiments, the AAV particle comprises a viral genome comprising SEQ ID NO: 2231 and a capsid comprising SEQ ID NO: 2679. In some embodiments, the AAV particle comprises a viral genome comprising SEQ ID NO: 2231 and a capsid comprising SEQ ID NO: 2809. In some embodiments, the AAV particle comprises a viral genome comprising SEQ ID NO: 2231 and a capsid comprising SEQ ID NO: 2871.

In some embodiments, the AAV particle comprises a viral genome comprising SEQ ID NO: 2232 and a VOY101 capsid. In some embodiments, the AAV particle comprises a viral genome comprising SEQ ID NO: 2232 and a VOY201 capsid. In some embodiments, the AAV particle comprises a viral genome comprising SEQ ID NO: 2232 and an AAV9 capsid. In some embodiments, the AAV particle comprises a viral genome comprising SEQ ID NO: 2232 and an AAV9K449R capsid. In some embodiments, the AAV particle comprises a viral genome comprising SEQ ID NO: 2232 and an AAVPHP.B capsid. In some embodiments, the AAV particle comprises a viral genome comprising SEQ ID NO: 2232 and an AAVPHP.N capsid. In some embodiments, the AAV particle comprises a viral genome comprising SEQ ID NO: 2232 and an AAV2 variant capsid. In some embodiments, the AAV particle comprises a viral genome comprising SEQ ID NO: 2232 and an AAV2/3 variant capsid. In some embodiments, the AAV particle comprises a viral genome comprising SEQ ID NO: 2232 and a capsid comprising SEQ ID NO: 1. In some embodiments, the AAV particle comprises a viral genome comprising SEQ ID NO: 2232 and a capsid encoded by a nucleic acid sequence comprising SEQ ID NO: 2. In some embodiments, the AAV particle comprises a viral genome comprising SEQ ID NO: 2232 and a capsid comprising SEQ ID NO: 4534. In some embodiments, the AAV particle comprises a viral genome comprising SEQ ID NO: 2232 and a capsid encoded by a nucleic acid sequence comprising SEQ ID NO: 3. In some embodiments, the AAV particle comprises a viral genome comprising SEQ ID NO: 2232 and a capsid comprising SEQ ID NO: 138. In some embodiments, the AAV particle comprises a viral genome comprising SEQ ID NO: 2232 and a capsid encoded by a nucleic acid sequence comprising SEQ ID NO: 137. In some embodiments, the AAV particle comprises a viral genome comprising SEQ ID NO: 2232 and a capsid comprising SEQ ID NO: 5. In some embodiments, the AAV particle comprises a viral genome comprising SEQ ID NO: 2232 and a capsid encoded by a nucleic acid sequence comprising SEQ ID NO: 6. In some embodiments, the AAV particle comprises a viral genome comprising SEQ ID NO: 2232 and a capsid comprising SEQ ID NO: 4. In some embodiments, the AAV particle comprises a viral genome comprising SEQ ID NO: 2232 and a capsid comprising SEQ ID NO: 11. In some embodiments, the AAV particle comprises a viral genome comprising SEQ ID NO: 2232 and a capsid comprising SEQ ID NO: 2679. In some embodiments, the AAV particle comprises a viral genome comprising SEQ ID NO: 2232 and a capsid comprising SEQ ID NO: 2809. In some embodiments, the AAV particle comprises a viral genome comprising SEQ ID NO: 2232 and a capsid comprising SEQ ID NO: 2871.

In some embodiments, the AAV particle comprises a viral genome comprising SEQ ID NO: 2233 and a VOY101 capsid. In some embodiments, the AAV particle comprises a viral genome comprising SEQ ID NO: 2233 and a VOY201 capsid. In some embodiments, the AAV particle comprises a viral genome comprising SEQ ID NO: 2233 and an AAV9 capsid. In some embodiments, the AAV particle comprises a viral genome comprising SEQ ID NO: 2233 and an AAV9K449R capsid. In some embodiments, the AAV particle comprises a viral genome comprising SEQ ID NO: 2233 and an AAVPHP.B capsid. In some embodiments, the AAV particle comprises a viral genome comprising SEQ ID NO: 2233 and an AAVPHP.N capsid. In some embodiments, the AAV particle comprises a viral genome comprising SEQ ID NO: 2233 and an AAV2 variant capsid. In some embodiments, the AAV particle comprises a viral genome comprising SEQ ID NO: 2233 and an AAV2/3 variant capsid. In some embodiments, the AAV particle comprises a viral genome comprising SEQ ID NO: 2233 and a capsid comprising SEQ ID NO: 1. In some embodiments, the AAV particle comprises a viral genome comprising SEQ ID NO: 2233 and a capsid encoded by a nucleic acid sequence comprising SEQ ID NO: 2. In some embodiments, the AAV particle comprises a viral genome comprising SEQ ID NO: 2233 and a capsid comprising SEQ ID NO: 4534. In some embodiments, the AAV particle comprises a viral genome comprising SEQ ID NO: 2233 and a capsid encoded by a nucleic acid sequence comprising SEQ ID NO: 3. In some embodiments, the AAV particle comprises a viral genome comprising SEQ ID NO: 2233 and a capsid comprising SEQ ID NO: 138. In some embodiments, the AAV particle comprises a viral genome comprising SEQ ID NO: 2233 and a capsid encoded by a nucleic acid sequence comprising SEQ ID NO: 137. In some embodiments, the AAV particle comprises a viral genome comprising SEQ ID NO: 2233 and a capsid comprising SEQ ID NO: 5. In some embodiments, the AAV particle comprises a viral genome comprising SEQ ID NO: 2233 and a capsid encoded by a nucleic acid sequence comprising SEQ ID NO: 6. In some embodiments, the AAV particle comprises a viral genome comprising SEQ ID NO: 2233 and a capsid comprising SEQ ID NO: 4. In some embodiments, the AAV particle comprises a viral genome comprising SEQ ID NO: 2233 and a capsid comprising SEQ ID NO: 11. In some embodiments, the AAV particle comprises a viral genome comprising SEQ ID NO: 2233 and a capsid comprising SEQ ID NO: 2679. In some embodiments, the AAV particle comprises a viral genome comprising SEQ ID NO: 2233 and a capsid comprising SEQ ID NO: 2809. In some embodiments, the AAV particle comprises a viral genome comprising SEQ ID NO: 2233 and a capsid comprising SEQ ID NO: 2871.

In some embodiments, the AAV particle comprises a viral genome comprising SEQ ID NO: 2234 and a VOY101 capsid. In some embodiments, the AAV particle comprises a viral genome comprising SEQ ID NO: 2234 and a VOY201 capsid. In some embodiments, the AAV particle comprises a viral genome comprising SEQ ID NO: 2234 and an AAV9 capsid. In some embodiments, the AAV particle comprises a viral genome comprising SEQ ID NO: 2234 and an AAV9K449R capsid. In some embodiments, the AAV particle comprises a viral genome comprising SEQ ID NO: 2234 and an AAVPHP.B capsid. In some embodiments, the AAV particle comprises a viral genome comprising SEQ ID NO: 2234 and an AAVPHP.N capsid. In some embodiments, the AAV particle comprises a viral genome comprising SEQ ID NO: 2234 and an AAV2 variant capsid. In some embodiments, the AAV particle comprises a viral genome comprising SEQ ID NO: 2234 and an AAV2/3 variant capsid. In some embodiments, the AAV particle comprises a viral genome comprising SEQ ID NO: 2234 and a capsid comprising SEQ ID NO: 1. In some embodiments, the AAV particle comprises a viral genome comprising SEQ ID NO: 2234 and a capsid encoded by a nucleic acid sequence comprising SEQ ID NO: 2. In some embodiments, the AAV particle comprises a viral genome comprising SEQ ID NO: 2234 and a capsid comprising SEQ ID NO: 4534. In some embodiments, the AAV particle comprises a viral genome comprising SEQ ID NO: 2234 and a capsid encoded by a nucleic acid sequence comprising SEQ ID NO: 3. In some embodiments, the AAV particle comprises a viral genome comprising SEQ ID NO: 2234 and a capsid comprising SEQ ID NO: 138. In some embodiments, the AAV particle comprises a viral genome comprising SEQ ID NO: 2234 and a capsid encoded by a nucleic acid sequence comprising SEQ ID NO: 137. In some embodiments, the AAV particle comprises a viral genome comprising SEQ ID NO: 2234 and a capsid comprising SEQ ID NO: 5. In some embodiments, the AAV particle comprises a viral genome comprising SEQ ID NO: 2234 and a capsid encoded by a nucleic acid sequence comprising SEQ ID NO: 6. In some embodiments, the AAV particle comprises a viral genome comprising SEQ ID NO: 2234 and a capsid comprising SEQ ID NO: 4. In some embodiments, the AAV particle comprises a viral genome comprising SEQ ID NO: 2234 and a capsid comprising SEQ ID NO: 11. In some embodiments, the AAV particle comprises a viral genome comprising SEQ ID NO: 2234 and a capsid comprising SEQ ID NO: 2679. In some embodiments, the AAV particle comprises a viral genome comprising SEQ ID NO: 2234 and a capsid comprising SEQ ID NO: 2809. In some embodiments, the AAV particle comprises a viral genome comprising SEQ ID NO: 2234 and a capsid comprising SEQ ID NO: 2871.

In some embodiments, the AAV particle comprises a viral genome comprising SEQ ID NO: 2235 and a VOY101 capsid. In some embodiments, the AAV particle comprises a viral genome comprising SEQ ID NO: 2235 and a VOY201 capsid. In some embodiments, the AAV particle comprises a viral genome comprising SEQ ID NO: 2235 and an AAV9 capsid. In some embodiments, the AAV particle comprises a viral genome comprising SEQ ID NO: 2235 and an AAV9K449R capsid. In some embodiments, the AAV particle comprises a viral genome comprising SEQ ID NO: 2235 and an AAVPHP.B capsid. In some embodiments, the AAV particle comprises a viral genome comprising SEQ ID NO: 2235 and an AAVPHP.N capsid. In some embodiments, the AAV particle comprises a viral genome comprising SEQ ID NO: 2235 and an AAV2 variant capsid. In some embodiments, the AAV particle comprises a viral genome comprising SEQ ID NO: 2235 and an AAV2/3 variant capsid. In some embodiments, the AAV particle comprises a viral genome comprising SEQ ID NO: 2235 and a capsid comprising SEQ ID NO: 1. In some embodiments, the AAV particle comprises a viral genome comprising SEQ ID NO: 2235 and a capsid encoded by a nucleic acid sequence comprising SEQ ID NO: 2. In some embodiments, the AAV particle comprises a viral genome comprising SEQ ID NO: 2235 and a capsid comprising SEQ ID NO: 4534. In some embodiments, the AAV particle comprises a viral genome comprising SEQ ID NO: 2235 and a capsid encoded by a nucleic acid sequence comprising SEQ ID NO: 3. In some embodiments, the AAV particle comprises a viral genome comprising SEQ ID NO: 2235 and a capsid comprising SEQ ID NO: 138. In some embodiments, the AAV particle comprises a viral genome comprising SEQ ID NO: 2235 and a capsid encoded by a nucleic acid sequence comprising SEQ ID NO: 137. In some embodiments, the AAV particle comprises a viral genome comprising SEQ ID NO: 2235 and a capsid comprising SEQ ID NO: 5. In some embodiments, the AAV particle comprises a viral genome comprising SEQ ID NO: 2235 and a capsid encoded by a nucleic acid sequence comprising SEQ ID NO: 6. In some embodiments, the AAV particle comprises a viral genome comprising SEQ ID NO: 2235 and a capsid comprising SEQ ID NO: 4. In some embodiments, the AAV particle comprises a viral genome comprising SEQ ID NO: 2235 and a capsid comprising SEQ ID NO: 11. In some embodiments, the AAV particle comprises a viral genome comprising SEQ ID NO: 2235 and a capsid comprising SEQ ID NO: 2679. In some embodiments, the AAV particle comprises a viral genome comprising SEQ ID NO: 2235 and a capsid comprising SEQ ID NO: 2809. In some embodiments, the AAV particle comprises a viral genome comprising SEQ ID NO: 2235 and a capsid comprising SEQ ID NO: 2871.

In some embodiments, the AAV particle comprises a viral genome comprising SEQ ID NO: 2236 and a VOY101 capsid. In some embodiments, the AAV particle comprises a viral genome comprising SEQ ID NO: 2236 and a VOY201 capsid. In some embodiments, the AAV particle comprises a viral genome comprising SEQ ID NO: 2236 and an AAV9 capsid. In some embodiments, the AAV particle comprises a viral genome comprising SEQ ID NO: 2236 and an AAV9K449R capsid. In some embodiments, the AAV particle comprises a viral genome comprising SEQ ID NO: 2236 and an AAVPHP.B capsid. In some embodiments, the AAV particle comprises a viral genome comprising SEQ ID NO: 2236 and an AAVPHP.N capsid. In some embodiments, the AAV particle comprises a viral genome comprising SEQ ID NO: 2236 and an AAV2 variant capsid. In some embodiments, the AAV particle comprises a viral genome comprising SEQ ID NO: 2236 and an AAV2/3 variant capsid. In some embodiments, the AAV particle comprises a viral genome comprising SEQ ID NO: 2236 and a capsid comprising SEQ ID NO: 1. In some embodiments, the AAV particle comprises a viral genome comprising SEQ ID NO: 2236 and a capsid encoded by a nucleic acid sequence comprising SEQ ID NO: 2. In some embodiments, the AAV particle comprises a viral genome comprising SEQ ID NO: 2236 and a capsid comprising SEQ ID NO: 4534. In some embodiments, the AAV particle comprises a viral genome comprising SEQ ID NO: 2236 and a capsid encoded by a nucleic acid sequence comprising SEQ ID NO: 3. In some embodiments, the AAV particle comprises a viral genome comprising SEQ ID NO: 2236 and a capsid comprising SEQ ID NO: 138. In some embodiments, the AAV particle comprises a viral genome comprising SEQ ID NO: 2236 and a capsid encoded by a nucleic acid sequence comprising SEQ ID NO: 137. In some embodiments, the AAV particle comprises a viral genome comprising SEQ ID NO: 2236 and a capsid comprising SEQ ID NO: 5. In some embodiments, the AAV particle comprises a viral genome comprising SEQ ID NO: 2236 and a capsid encoded by a nucleic acid sequence comprising SEQ ID NO: 6. In some embodiments, the AAV particle comprises a viral genome comprising SEQ ID NO: 2236 and a capsid comprising SEQ ID NO: 4. In some embodiments, the AAV particle comprises a viral genome comprising SEQ ID NO: 2236 and a capsid comprising SEQ ID NO: 11. In some embodiments, the AAV particle comprises a viral genome comprising SEQ ID NO: 2236 and a capsid comprising SEQ ID NO: 2679. In some embodiments, the AAV particle comprises a viral genome comprising SEQ ID NO: 2236 and a capsid comprising SEQ ID NO: 2809. In some embodiments, the AAV particle comprises a viral genome comprising SEQ ID NO: 2236 and a capsid comprising SEQ ID NO: 2871.

In some embodiments, the AAV particle comprises a viral genome comprising SEQ ID NO: 2237 and a VOY101 capsid. In some embodiments, the AAV particle comprises a viral genome comprising SEQ ID NO: 2237 and a VOY201 capsid. In some embodiments, the AAV particle comprises a viral genome comprising SEQ ID NO: 2237 and an AAV9 capsid. In some embodiments, the AAV particle comprises a viral genome comprising SEQ ID NO: 2237 and an AAV9K449R capsid. In some embodiments, the AAV particle comprises a viral genome comprising SEQ ID NO: 2237 and an AAVPHP.B capsid. In some embodiments, the AAV particle comprises a viral genome comprising SEQ ID NO: 2237 and an AAVPHP.N capsid. In some embodiments, the AAV particle comprises a viral genome comprising SEQ ID NO: 2237 and an AAV2 variant capsid. In some embodiments, the AAV particle comprises a viral genome comprising SEQ ID NO: 2237 and an AAV2/3 variant capsid. In some embodiments, the AAV particle comprises a viral genome comprising SEQ ID NO: 2237 and a capsid comprising SEQ ID NO: 1. In some embodiments, the AAV particle comprises a viral genome comprising SEQ ID NO: 2237 and a capsid encoded by a nucleic acid sequence comprising SEQ ID NO: 2. In some embodiments, the AAV particle comprises a viral genome comprising SEQ ID NO: 2237 and a capsid comprising SEQ ID NO: 4534. In some embodiments, the AAV particle comprises a viral genome comprising SEQ ID NO: 2237 and a capsid encoded by a nucleic acid sequence comprising SEQ ID NO: 3. In some embodiments, the AAV particle comprises a viral genome comprising SEQ ID NO: 2237 and a capsid comprising SEQ ID NO: 138. In some embodiments, the AAV particle comprises a viral genome comprising SEQ ID NO: 2237 and a capsid encoded by a nucleic acid sequence comprising SEQ ID NO: 137. In some embodiments, the AAV particle comprises a viral genome comprising SEQ ID NO: 2237 and a capsid comprising SEQ ID NO: 5. In some embodiments, the AAV particle comprises a viral genome comprising SEQ ID NO: 2237 and a capsid encoded by a nucleic acid sequence comprising SEQ ID NO: 6. In some embodiments, the AAV particle comprises a viral genome comprising SEQ ID NO: 2237 and a capsid comprising SEQ ID NO: 4. In some embodiments, the AAV particle comprises a viral genome comprising SEQ ID NO: 2237 and a capsid comprising SEQ ID NO: 11. In some embodiments, the AAV particle comprises a viral genome comprising SEQ ID NO: 2237 and a capsid comprising SEQ ID NO: 2679. In some embodiments, the AAV particle comprises a viral genome comprising SEQ ID NO: 2237 and a capsid comprising SEQ ID NO: 2809. In some embodiments, the AAV particle comprises a viral genome comprising SEQ ID NO: 2237 and a capsid comprising SEQ ID NO: 2871.

In some embodiments, the AAV particle comprises a viral genome comprising SEQ ID NO: 2260 and a VOY101 capsid. In some embodiments, the AAV particle comprises a viral genome comprising SEQ ID NO: 2260 and a VOY201 capsid. In some embodiments, the AAV particle comprises a viral genome comprising SEQ ID NO: 2260 and an AAV9 capsid. In some embodiments, the AAV particle comprises a viral genome comprising SEQ ID NO: 2260 and an AAV9K449R capsid. In some embodiments, the AAV particle comprises a viral genome comprising SEQ ID NO: 2260 and an AAVPHP.B capsid. In some embodiments, the AAV particle comprises a viral genome comprising SEQ ID NO: 2260 and an AAVPHP.N capsid. In some embodiments, the AAV particle comprises a viral genome comprising SEQ ID NO: 2260 and an AAV2 variant capsid. In some embodiments, the AAV particle comprises a viral genome comprising SEQ ID NO: 2260 and an AAV2/3 variant capsid. In some embodiments, the AAV particle comprises a viral genome comprising SEQ ID NO: 2260 and a capsid comprising SEQ ID NO: 1. In some embodiments, the AAV particle comprises a viral genome comprising SEQ ID NO: 2260 and a capsid encoded by a nucleic acid sequence comprising SEQ ID NO: 2. In some embodiments, the AAV particle comprises a viral genome comprising SEQ ID NO: 2260 and a capsid comprising SEQ ID NO: 4534. In some embodiments, the AAV particle comprises a viral genome comprising SEQ ID NO: 2260 and a capsid encoded by a nucleic acid sequence comprising SEQ ID NO: 3. In some embodiments, the AAV particle comprises a viral genome comprising SEQ ID NO: 2260 and a capsid comprising SEQ ID NO: 138. In some embodiments, the AAV particle comprises a viral genome comprising SEQ ID NO: 2260 and a capsid encoded by a nucleic acid sequence comprising SEQ ID NO: 137. In some embodiments, the AAV particle comprises a viral genome comprising SEQ ID NO: 2260 and a capsid comprising SEQ ID NO: 5. In some embodiments, the AAV particle comprises a viral genome comprising SEQ ID NO: 2260 and a capsid encoded by a nucleic acid sequence comprising SEQ ID NO: 6. In some embodiments, the AAV particle comprises a viral genome comprising SEQ ID NO: 2260 and a capsid comprising SEQ ID NO: 4. In some embodiments, the AAV particle comprises a viral genome comprising SEQ ID NO: 2260 and a capsid comprising SEQ ID NO: 11. In some embodiments, the AAV particle comprises a viral genome comprising SEQ ID NO: 2260 and a capsid comprising SEQ ID NO: 2679. In some embodiments, the AAV particle comprises a viral genome comprising SEQ ID NO: 2260 and a capsid comprising SEQ ID NO: 2809. In some embodiments, the AAV particle comprises a viral genome comprising SEQ ID NO: 2260 and a capsid comprising SEQ ID NO: 2871.

In some embodiments, the AAV particle comprises a viral genome comprising SEQ ID NO: 2261 and a VOY101 capsid. In some embodiments, the AAV particle comprises a viral genome comprising SEQ ID NO: 2261 and a VOY201 capsid. In some embodiments, the AAV particle comprises a viral genome comprising SEQ ID NO: 2261 and an AAV9 capsid. In some embodiments, the AAV particle comprises a viral genome comprising SEQ ID NO: 2261 and an AAV9K449R capsid. In some embodiments, the AAV particle comprises a viral genome comprising SEQ ID NO: 2261 and an AAVPHP.B capsid. In some embodiments, the AAV particle comprises a viral genome comprising SEQ ID NO: 2261 and an AAVPHP.N capsid. In some embodiments, the AAV particle comprises a viral genome comprising SEQ ID NO: 2261 and an AAV2 variant capsid. In some embodiments, the AAV particle comprises a viral genome comprising SEQ ID NO: 2261 and an AAV2/3 variant capsid. In some embodiments, the AAV particle comprises a viral genome comprising SEQ ID NO: 2261 and a capsid comprising SEQ ID NO: 1. In some embodiments, the AAV particle comprises a viral genome comprising SEQ ID NO: 2261 and a capsid encoded by a nucleic acid sequence comprising SEQ ID NO: 2. In some embodiments, the AAV particle comprises a viral genome comprising SEQ ID NO: 2261 and a capsid comprising SEQ ID NO: 4534. In some embodiments, the AAV particle comprises a viral genome comprising SEQ ID NO: 2261 and a capsid encoded by a nucleic acid sequence comprising SEQ ID NO: 3. In some embodiments, the AAV particle comprises a viral genome comprising SEQ ID NO: 2261 and a capsid comprising SEQ ID NO: 138. In some embodiments, the AAV particle comprises a viral genome comprising SEQ ID NO: 2261 and a capsid encoded by a nucleic acid sequence comprising SEQ ID NO: 137. In some embodiments, the AAV particle comprises a viral genome comprising SEQ ID NO: 2261 and a capsid comprising SEQ ID NO: 5. In some embodiments, the AAV particle comprises a viral genome comprising SEQ ID NO: 2261 and a capsid encoded by a nucleic acid sequence comprising SEQ ID NO: 6. In some embodiments, the AAV particle comprises a viral genome comprising SEQ ID NO: 2261 and a capsid comprising SEQ ID NO: 4. In some embodiments, the AAV particle comprises a viral genome comprising SEQ ID NO: 2261 and a capsid comprising SEQ ID NO: 11. In some embodiments, the AAV particle comprises a viral genome comprising SEQ ID NO: 2261 and a capsid comprising SEQ ID NO: 2679. In some embodiments, the AAV particle comprises a viral genome comprising SEQ ID NO: 2261 and a capsid comprising SEQ ID NO: 2809. In some embodiments, the AAV particle comprises a viral genome comprising SEQ ID NO: 2261 and a capsid comprising SEQ ID NO: 2871.

In some embodiments, the AAV particle comprises a viral genome comprising SEQ ID NO: 2262 and a VOY101 capsid. In some embodiments, the AAV particle comprises a viral genome comprising SEQ ID NO: 2262 and a VOY201 capsid. In some embodiments, the AAV particle comprises a viral genome comprising SEQ ID NO: 2262 and an AAV9 capsid. In some embodiments, the AAV particle comprises a viral genome comprising SEQ ID NO: 2262 and an AAV9K449R capsid. In some embodiments, the AAV particle comprises a viral genome comprising SEQ ID NO: 2262 and an AAVPHP.B capsid. In some embodiments, the AAV particle comprises a viral genome comprising SEQ ID NO: 2262 and an AAVPHP.N capsid. In some embodiments, the AAV particle comprises a viral genome comprising SEQ ID NO: 2262 and an AAV2 variant capsid. In some embodiments, the AAV particle comprises a viral genome comprising SEQ ID NO: 2262 and an AAV2/3 variant capsid. In some embodiments, the AAV particle comprises a viral genome comprising SEQ ID NO: 2262 and a capsid comprising SEQ ID NO: 1. In some embodiments, the AAV particle comprises a viral genome comprising SEQ ID NO: 2262 and a capsid encoded by a nucleic acid sequence comprising SEQ ID NO: 2. In some embodiments, the AAV particle comprises a viral genome comprising SEQ ID NO: 2262 and a capsid comprising SEQ ID NO: 4534. In some embodiments, the AAV particle comprises a viral genome comprising SEQ ID NO: 2262 and a capsid encoded by a nucleic acid sequence comprising SEQ ID NO: 3. In some embodiments, the AAV particle comprises a viral genome comprising SEQ ID NO: 2262 and a capsid comprising SEQ ID NO: 138. In some embodiments, the AAV particle comprises a viral genome comprising SEQ ID NO: 2262 and a capsid encoded by a nucleic acid sequence comprising SEQ ID NO: 137. In some embodiments, the AAV particle comprises a viral genome comprising SEQ ID NO: 2262 and a capsid comprising SEQ ID NO: 5. In some embodiments, the AAV particle comprises a viral genome comprising SEQ ID NO: 2262 and a capsid encoded by a nucleic acid sequence comprising SEQ ID NO: 6. In some embodiments, the AAV particle comprises a viral genome comprising SEQ ID NO: 2262 and a capsid comprising SEQ ID NO: 4. In some embodiments, the AAV particle comprises a viral genome comprising SEQ ID NO: 2262 and a capsid comprising SEQ ID NO: 11. In some embodiments, the AAV particle comprises a viral genome comprising SEQ ID NO: 2262 and a capsid comprising SEQ ID NO: 2679. In some embodiments, the AAV particle comprises a viral genome comprising SEQ ID NO: 2262 and a capsid comprising SEQ ID NO: 2809. In some embodiments, the AAV particle comprises a viral genome comprising SEQ ID NO: 2262 and a capsid comprising SEQ ID NO: 2871.

In some embodiments, the AAV particle comprises a viral genome comprising SEQ ID NO: 2263 and a VOY101 capsid. In some embodiments, the AAV particle comprises a viral genome comprising SEQ ID NO: 2263 and a VOY201 capsid. In some embodiments, the AAV particle comprises a viral genome comprising SEQ ID NO: 2263 and an AAV9 capsid. In some embodiments, the AAV particle comprises a viral genome comprising SEQ ID NO: 2263 and an AAV9K449R capsid. In some embodiments, the AAV particle comprises a viral genome comprising SEQ ID NO: 2263 and an AAVPHP.B capsid. In some embodiments, the AAV particle comprises a viral genome comprising SEQ ID NO: 2263 and an AAVPHP.N capsid. In some embodiments, the AAV particle comprises a viral genome comprising SEQ ID NO: 2263 and an AAV2 variant capsid. In some embodiments, the AAV particle comprises a viral genome comprising SEQ ID NO: 2263 and an AAV2/3 variant capsid. In some embodiments, the AAV particle comprises a viral genome comprising SEQ ID NO: 2263 and a capsid comprising SEQ ID NO: 1. In some embodiments, the AAV particle comprises a viral genome comprising SEQ ID NO: 2263 and a capsid encoded by a nucleic acid sequence comprising SEQ ID NO: 2. In some embodiments, the AAV particle comprises a viral genome comprising SEQ ID NO: 2263 and a capsid comprising SEQ ID NO: 4534. In some embodiments, the AAV particle comprises a viral genome comprising SEQ ID NO: 2263 and a capsid encoded by a nucleic acid sequence comprising SEQ ID NO: 3. In some embodiments, the AAV particle comprises a viral genome comprising SEQ ID NO: 2263 and a capsid comprising SEQ ID NO: 138. In some embodiments, the AAV particle comprises a viral genome comprising SEQ ID NO: 2263 and a capsid encoded by a nucleic acid sequence comprising SEQ ID NO: 137. In some embodiments, the AAV particle comprises a viral genome comprising SEQ ID NO: 2263 and a capsid comprising SEQ ID NO: 5. In some embodiments, the AAV particle comprises a viral genome comprising SEQ ID NO: 2263 and a capsid encoded by a nucleic acid sequence comprising SEQ ID NO: 6. In some embodiments, the AAV particle comprises a viral genome comprising SEQ ID NO: 2263 and a capsid comprising SEQ ID NO: 4. In some embodiments, the AAV particle comprises a viral genome comprising SEQ ID NO: 2263 and a capsid comprising SEQ ID NO: 11. In some embodiments, the AAV particle comprises a viral genome comprising SEQ ID NO: 2263 and a capsid comprising SEQ ID NO: 2679. In some embodiments, the AAV particle comprises a viral genome comprising SEQ ID NO: 2263 and a capsid comprising SEQ ID NO: 2809. In some embodiments, the AAV particle comprises a viral genome comprising SEQ ID NO: 2263 and a capsid comprising SEQ ID NO: 2871.

In some embodiments, the AAV particle comprises a viral genome comprising SEQ ID NO: 2264 and a VOY101 capsid. In some embodiments, the AAV particle comprises a viral genome comprising SEQ ID NO: 2264 and a VOY201 capsid. In some embodiments, the AAV particle comprises a viral genome comprising SEQ ID NO: 2264 and an AAV9 capsid. In some embodiments, the AAV particle comprises a viral genome comprising SEQ ID NO: 2264 and an AAV9K449R capsid. In some embodiments, the AAV particle comprises a viral genome comprising SEQ ID NO: 2264 and an AAVPHP.B capsid. In some embodiments, the AAV particle comprises a viral genome comprising SEQ ID NO: 2264 and an AAVPHP.N capsid. In some embodiments, the AAV particle comprises a viral genome comprising SEQ ID NO: 2264 and an AAV2 variant capsid. In some embodiments, the AAV particle comprises a viral genome comprising SEQ ID NO: 2264 and an AAV2/3 variant capsid. In some embodiments, the AAV particle comprises a viral genome comprising SEQ ID NO: 2264 and a capsid comprising SEQ ID NO: 1. In some embodiments, the AAV particle comprises a viral genome comprising SEQ ID NO: 2264 and a capsid encoded by a nucleic acid sequence comprising SEQ ID NO: 2. In some embodiments, the AAV particle comprises a viral genome comprising SEQ ID NO: 2264 and a capsid comprising SEQ ID NO: 4534. In some embodiments, the AAV particle comprises a viral genome comprising SEQ ID NO: 2264 and a capsid encoded by a nucleic acid sequence comprising SEQ ID NO: 3. In some embodiments, the AAV particle comprises a viral genome comprising SEQ ID NO: 2264 and a capsid comprising SEQ ID NO: 138. In some embodiments, the AAV particle comprises a viral genome comprising SEQ ID NO: 2264 and a capsid encoded by a nucleic acid sequence comprising SEQ ID NO: 137. In some embodiments, the AAV particle comprises a viral genome comprising SEQ ID NO: 2264 and a capsid comprising SEQ ID NO: 5. In some embodiments, the AAV particle comprises a viral genome comprising SEQ ID NO: 2264 and a capsid encoded by a nucleic acid sequence comprising SEQ ID NO: 6. In some embodiments, the AAV particle comprises a viral genome comprising SEQ ID NO: 2264 and a capsid comprising SEQ ID NO: 4. In some embodiments, the AAV particle comprises a viral genome comprising SEQ ID NO: 2264 and a capsid comprising SEQ ID NO: 11. In some embodiments, the AAV particle comprises a viral genome comprising SEQ ID NO: 2264 and a capsid comprising SEQ ID NO: 2679. In some embodiments, the AAV particle comprises a viral genome comprising SEQ ID NO: 2264 and a capsid comprising SEQ ID NO: 2809. In some embodiments, the AAV particle comprises a viral genome comprising SEQ ID NO: 2264 and a capsid comprising SEQ ID NO: 2871.

In some embodiments, the AAV particle comprises a viral genome comprising SEQ ID NO: 2265 and a VOY101 capsid. In some embodiments, the AAV particle comprises a viral genome comprising SEQ ID NO: 2265 and a VOY201 capsid. In some embodiments, the AAV particle comprises a viral genome comprising SEQ ID NO: 2265 and an AAV9 capsid. In some embodiments, the AAV particle comprises a viral genome comprising SEQ ID NO: 2265 and an AAV9K449R capsid. In some embodiments, the AAV particle comprises a viral genome comprising SEQ ID NO: 2265 and an AAVPHP.B capsid. In some embodiments, the AAV particle comprises a viral genome comprising SEQ ID NO: 2265 and an AAVPHP.N capsid. In some embodiments, the AAV particle comprises a viral genome comprising SEQ ID NO: 2265 and an AAV2 variant capsid. In some embodiments, the AAV particle comprises a viral genome comprising SEQ ID NO: 2265 and an AAV2/3 variant capsid. In some embodiments, the AAV particle comprises a viral genome comprising SEQ ID NO: 2265 and a capsid comprising SEQ ID NO: 1. In some embodiments, the AAV particle comprises a viral genome comprising SEQ ID NO: 2265 and a capsid encoded by a nucleic acid sequence comprising SEQ ID NO: 2. In some embodiments, the AAV particle comprises a viral genome comprising SEQ ID NO: 2265 and a capsid comprising SEQ ID NO: 4534. In some embodiments, the AAV particle comprises a viral genome comprising SEQ ID NO: 2265 and a capsid encoded by a nucleic acid sequence comprising SEQ ID NO: 3. In some embodiments, the AAV particle comprises a viral genome comprising SEQ ID NO: 2265 and a capsid comprising SEQ ID NO: 138. In some embodiments, the AAV particle comprises a viral genome comprising SEQ ID NO: 2265 and a capsid encoded by a nucleic acid sequence comprising SEQ ID NO: 137. In some embodiments, the AAV particle comprises a viral genome comprising SEQ ID NO: 2265 and a capsid comprising SEQ ID NO: 5. In some embodiments, the AAV particle comprises a viral genome comprising SEQ ID NO: 2265 and a capsid encoded by a nucleic acid sequence comprising SEQ ID NO: 6. In some embodiments, the AAV particle comprises a viral genome comprising SEQ ID NO: 2265 and a capsid comprising SEQ ID NO: 4. In some embodiments, the AAV particle comprises a viral genome comprising SEQ ID NO: 2265 and a capsid comprising SEQ ID NO: 11. In some embodiments, the AAV particle comprises a viral genome comprising SEQ ID NO: 2265 and a capsid comprising SEQ ID NO: 2679. In some embodiments, the AAV particle comprises a viral genome comprising SEQ ID NO: 2265 and a capsid comprising SEQ ID NO: 2809. In some embodiments, the AAV particle comprises a viral genome comprising SEQ ID NO: 2265 and a capsid comprising SEQ ID NO: 2871.

In some embodiments, the AAV particle comprises a viral genome comprising SEQ ID NO: 2266 and a VOY101 capsid. In some embodiments, the AAV particle comprises a viral genome comprising SEQ ID NO: 2266 and a VOY201 capsid. In some embodiments, the AAV particle comprises a viral genome comprising SEQ ID NO: 2266 and an AAV9 capsid. In some embodiments, the AAV particle comprises a viral genome comprising SEQ ID NO: 2266 and an AAV9K449R capsid. In some embodiments, the AAV particle comprises a viral genome comprising SEQ ID NO: 2266 and an AAVPHP.B capsid. In some embodiments, the AAV particle comprises a viral genome comprising SEQ ID NO: 2266 and an AAVPHP.N capsid. In some embodiments, the AAV particle comprises a viral genome comprising SEQ ID NO: 2266 and an AAV2 variant capsid. In some embodiments, the AAV particle comprises a viral genome comprising SEQ ID NO: 2266 and an AAV2/3 variant capsid. In some embodiments, the AAV particle comprises a viral genome comprising SEQ ID NO: 2266 and a capsid comprising SEQ ID NO: 1. In some embodiments, the AAV particle comprises a viral genome comprising SEQ ID NO: 2266 and a capsid encoded by a nucleic acid sequence comprising SEQ ID NO: 2. In some embodiments, the AAV particle comprises a viral genome comprising SEQ ID NO: 2266 and a capsid comprising SEQ ID NO: 4534. In some embodiments, the AAV particle comprises a viral genome comprising SEQ ID NO: 2266 and a capsid encoded by a nucleic acid sequence comprising SEQ ID NO: 3. In some embodiments, the AAV particle comprises a viral genome comprising SEQ ID NO: 2266 and a capsid comprising SEQ ID NO: 138. In some embodiments, the AAV particle comprises a viral genome comprising SEQ ID NO: 2266 and a capsid encoded by a nucleic acid sequence comprising SEQ ID NO: 137. In some embodiments, the AAV particle comprises a viral genome comprising SEQ ID NO: 2266 and a capsid comprising SEQ ID NO: 5. In some embodiments, the AAV particle comprises a viral genome comprising SEQ ID NO: 2266 and a capsid encoded by a nucleic acid sequence comprising SEQ ID NO: 6. In some embodiments, the AAV particle comprises a viral genome comprising SEQ ID NO: 2266 and a capsid comprising SEQ ID NO: 4. In some embodiments, the AAV particle comprises a viral genome comprising SEQ ID NO: 2266 and a capsid comprising SEQ ID NO: 11. In some embodiments, the AAV particle comprises a viral genome comprising SEQ ID NO: 2266 and a capsid comprising SEQ ID NO: 2679. In some embodiments, the AAV particle comprises a viral genome comprising SEQ ID NO: 2266 and a capsid comprising SEQ ID NO: 2809. In some embodiments, the AAV particle comprises a viral genome comprising SEQ ID NO: 2266 and a capsid comprising SEQ ID NO: 2871.

In some embodiments, the AAV particle comprises a viral genome comprising SEQ ID NO: 2267 and a VOY101 capsid. In some embodiments, the AAV particle comprises a viral genome comprising SEQ ID NO: 2267 and a VOY201 capsid. In some embodiments, the AAV particle comprises a viral genome comprising SEQ ID NO: 2267 and an AAV9 capsid. In some embodiments, the AAV particle comprises a viral genome comprising SEQ ID NO: 2267 and an AAV9K449R capsid. In some embodiments, the AAV particle comprises a viral genome comprising SEQ ID NO: 2267 and an AAVPHP.B capsid. In some embodiments, the AAV particle comprises a viral genome comprising SEQ ID NO: 2267 and an AAVPHP.N capsid. In some embodiments, the AAV particle comprises a viral genome comprising SEQ ID NO: 2267 and an AAV2 variant capsid. In some embodiments, the AAV particle comprises a viral genome comprising SEQ ID NO: 2267 and an AAV2/3 variant capsid. In some embodiments, the AAV particle comprises a viral genome comprising SEQ ID NO: 2267 and a capsid comprising SEQ ID NO: 1. In some embodiments, the AAV particle comprises a viral genome comprising SEQ ID NO: 2267 and a capsid encoded by a nucleic acid sequence comprising SEQ ID NO: 2. In some embodiments, the AAV particle comprises a viral genome comprising SEQ ID NO: 2267 and a capsid comprising SEQ ID NO: 4534. In some embodiments, the AAV particle comprises a viral genome comprising SEQ ID NO: 2267 and a capsid encoded by a nucleic acid sequence comprising SEQ ID NO: 3. In some embodiments, the AAV particle comprises a viral genome comprising SEQ ID NO: 2267 and a capsid comprising SEQ ID NO: 138. In some embodiments, the AAV particle comprises a viral genome comprising SEQ ID NO: 2267 and a capsid encoded by a nucleic acid sequence comprising SEQ ID NO: 137. In some embodiments, the AAV particle comprises a viral genome comprising SEQ ID NO: 2267 and a capsid comprising SEQ ID NO: 5. In some embodiments, the AAV particle comprises a viral genome comprising SEQ ID NO: 2267 and a capsid encoded by a nucleic acid sequence comprising SEQ ID NO: 6. In some embodiments, the AAV particle comprises a viral genome comprising SEQ ID NO: 2267 and a capsid comprising SEQ ID NO: 4. In some embodiments, the AAV particle comprises a viral genome comprising SEQ ID NO: 2267 and a capsid comprising SEQ ID NO: 11. In some embodiments, the AAV particle comprises a viral genome comprising SEQ ID NO: 2267 and a capsid comprising SEQ ID NO: 2679. In some embodiments, the AAV particle comprises a viral genome comprising SEQ ID NO: 2267 and a capsid comprising SEQ ID NO: 2809. In some embodiments, the AAV particle comprises a viral genome comprising SEQ ID NO: 2267 and a capsid comprising SEQ ID NO: 2871.

In some embodiments, the AAV particle comprises a viral genome comprising SEQ ID NO: 2268 and a VOY101 capsid. In some embodiments, the AAV particle comprises a viral genome comprising SEQ ID NO: 2268 and a VOY201 capsid. In some embodiments, the AAV particle comprises a viral genome comprising SEQ ID NO: 2268 and an AAV9 capsid. In some embodiments, the AAV particle comprises a viral genome comprising SEQ ID NO: 2268 and an AAV9K449R capsid. In some embodiments, the AAV particle comprises a viral genome comprising SEQ ID NO: 2268 and an AAVPHP.B capsid. In some embodiments, the AAV particle comprises a viral genome comprising SEQ ID NO: 2268 and an AAVPHP.N capsid. In some embodiments, the AAV particle comprises a viral genome comprising SEQ ID NO: 2268 and an AAV2 variant capsid. In some embodiments, the AAV particle comprises a viral genome comprising SEQ ID NO: 2268 and an AAV2/3 variant capsid. In some embodiments, the AAV particle comprises a viral genome comprising SEQ ID NO: 2268 and a capsid comprising SEQ ID NO: 1. In some embodiments, the AAV particle comprises a viral genome comprising SEQ ID NO: 2268 and a capsid encoded by a nucleic acid sequence comprising SEQ ID NO: 2. In some embodiments, the AAV particle comprises a viral genome comprising SEQ ID NO: 2268 and a capsid comprising SEQ ID NO: 4534. In some embodiments, the AAV particle comprises a viral genome comprising SEQ ID NO: 2268 and a capsid encoded by a nucleic acid sequence comprising SEQ ID NO: 3. In some embodiments, the AAV particle comprises a viral genome comprising SEQ ID NO: 2268 and a capsid comprising SEQ ID NO: 138. In some embodiments, the AAV particle comprises a viral genome comprising SEQ ID NO: 2268 and a capsid encoded by a nucleic acid sequence comprising SEQ ID NO: 137. In some embodiments, the AAV particle comprises a viral genome comprising SEQ ID NO: 2268 and a capsid comprising SEQ ID NO: 5. In some embodiments, the AAV particle comprises a viral genome comprising SEQ ID NO: 2268 and a capsid encoded by a nucleic acid sequence comprising SEQ ID NO: 6. In some embodiments, the AAV particle comprises a viral genome comprising SEQ ID NO: 2268 and a capsid comprising SEQ ID NO: 4. In some embodiments, the AAV particle comprises a viral genome comprising SEQ ID NO: 2268 and a capsid comprising SEQ ID NO: 11. In some embodiments, the AAV particle comprises a viral genome comprising SEQ ID NO: 2268 and a capsid comprising SEQ ID NO: 2679. In some embodiments, the AAV particle comprises a viral genome comprising SEQ ID NO: 2268 and a capsid comprising SEQ ID NO: 2809. In some embodiments, the AAV particle comprises a viral genome comprising SEQ ID NO: 2268 and a capsid comprising SEQ ID NO: 2871.

In some embodiments, the AAV particle comprises a viral genome comprising SEQ ID NO: 2269 and a VOY101 capsid. In some embodiments, the AAV particle comprises a viral genome comprising SEQ ID NO: 2269 and a VOY201 capsid. In some embodiments, the AAV particle comprises a viral genome comprising SEQ ID NO: 2269 and an AAV9 capsid. In some embodiments, the AAV particle comprises a viral genome comprising SEQ ID NO: 2269 and an AAV9K449R capsid. In some embodiments, the AAV particle comprises a viral genome comprising SEQ ID NO: 2269 and an AAVPHP.B capsid. In some embodiments, the AAV particle comprises a viral genome comprising SEQ ID NO: 2269 and an AAVPHP.N capsid. In some embodiments, the AAV particle comprises a viral genome comprising SEQ ID NO: 2269 and an AAV2 variant capsid. In some embodiments, the AAV particle comprises a viral genome comprising SEQ ID NO: 2269 and an AAV2/3 variant capsid. In some embodiments, the AAV particle comprises a viral genome comprising SEQ ID NO: 2269 and a capsid comprising SEQ ID NO: 1. In some embodiments, the AAV particle comprises a viral genome comprising SEQ ID NO: 2269 and a capsid encoded by a nucleic acid sequence comprising SEQ ID NO: 2. In some embodiments, the AAV particle comprises a viral genome comprising SEQ ID NO: 2269 and a capsid comprising SEQ ID NO: 4534. In some embodiments, the AAV particle comprises a viral genome comprising SEQ ID NO: 2269 and a capsid encoded by a nucleic acid sequence comprising SEQ ID NO: 3. In some embodiments, the AAV particle comprises a viral genome comprising SEQ ID NO: 2269 and a capsid comprising SEQ ID NO: 138. In some embodiments, the AAV particle comprises a viral genome comprising SEQ ID NO: 2269 and a capsid encoded by a nucleic acid sequence comprising SEQ ID NO: 137. In some embodiments, the AAV particle comprises a viral genome comprising SEQ ID NO: 2269 and a capsid comprising SEQ ID NO: 5. In some embodiments, the AAV particle comprises a viral genome comprising SEQ ID NO: 2269 and a capsid encoded by a nucleic acid sequence comprising SEQ ID NO: 6. In some embodiments, the AAV particle comprises a viral genome comprising SEQ ID NO: 2269 and a capsid comprising SEQ ID NO: 4. In some embodiments, the AAV particle comprises a viral genome comprising SEQ ID NO: 2269 and a capsid comprising SEQ ID NO: 11. In some embodiments, the AAV particle comprises a viral genome comprising SEQ ID NO: 2269 and a capsid comprising SEQ ID NO: 2679. In some embodiments, the AAV particle comprises a viral genome comprising SEQ ID NO: 2269 and a capsid comprising SEQ ID NO: 2809. In some embodiments, the AAV particle comprises a viral genome comprising SEQ ID NO: 2269 and a capsid comprising SEQ ID NO: 2871.

In some embodiments, the AAV particle comprises a viral genome comprising SEQ ID NO: 2270 and a VOY101 capsid. In some embodiments, the AAV particle comprises a viral genome comprising SEQ ID NO: 2270 and a VOY201 capsid. In some embodiments, the AAV particle comprises a viral genome comprising SEQ ID NO: 2270 and an AAV9 capsid. In some embodiments, the AAV particle comprises a viral genome comprising SEQ ID NO: 2270 and an AAV9K449R capsid. In some embodiments, the AAV particle comprises a viral genome comprising SEQ ID NO: 2270 and an AAVPHP.B capsid. In some embodiments, the AAV particle comprises a viral genome comprising SEQ ID NO: 2270 and an AAVPHP.N capsid. In some embodiments, the AAV particle comprises a viral genome comprising SEQ ID NO: 2270 and an AAV2 variant capsid. In some embodiments, the AAV particle comprises a viral genome comprising SEQ ID NO: 2270 and an AAV2/3 variant capsid. In some embodiments, the AAV particle comprises a viral genome comprising SEQ ID NO: 2270 and a capsid comprising SEQ ID NO: 1. In some embodiments, the AAV particle comprises a viral genome comprising SEQ ID NO: 2270 and a capsid encoded by a nucleic acid sequence comprising SEQ ID NO: 2. In some embodiments, the AAV particle comprises a viral genome comprising SEQ ID NO: 2270 and a capsid comprising SEQ ID NO: 4534. In some embodiments, the AAV particle comprises a viral genome comprising SEQ ID NO: 2270 and a capsid encoded by a nucleic acid sequence comprising SEQ ID NO: 3. In some embodiments, the AAV particle comprises a viral genome comprising SEQ ID NO: 2270 and a capsid comprising SEQ ID NO: 138. In some embodiments, the AAV particle comprises a viral genome comprising SEQ ID NO: 2270 and a capsid encoded by a nucleic acid sequence comprising SEQ ID NO: 137. In some embodiments, the AAV particle comprises a viral genome comprising SEQ ID NO: 2270 and a capsid comprising SEQ ID NO: 5. In some embodiments, the AAV particle comprises a viral genome comprising SEQ ID NO: 2270 and a capsid encoded by a nucleic acid sequence comprising SEQ ID NO: 6. In some embodiments, the AAV particle comprises a viral genome comprising SEQ ID NO: 2270 and a capsid comprising SEQ ID NO: 4. In some embodiments, the AAV particle comprises a viral genome comprising SEQ ID NO: 2270 and a capsid comprising SEQ ID NO: 11. In some embodiments, the AAV particle comprises a viral genome comprising SEQ ID NO: 2270 and a capsid comprising SEQ ID NO: 2679. In some embodiments, the AAV particle comprises a viral genome comprising SEQ ID NO: 2270 and a capsid comprising SEQ ID NO: 2809. In some embodiments, the AAV particle comprises a viral genome comprising SEQ ID NO: 2270 and a capsid comprising SEQ ID NO: 2871.

In some embodiments, the AAV particle comprises a viral genome comprising SEQ ID NO: 2271 and a VOY101 capsid. In some embodiments, the AAV particle comprises a viral genome comprising SEQ ID NO: 2271 and a VOY201 capsid. In some embodiments, the AAV particle comprises a viral genome comprising SEQ ID NO: 2271 and an AAV9 capsid. In some embodiments, the AAV particle comprises a viral genome comprising SEQ ID NO: 2271 and an AAV9K449R capsid. In some embodiments, the AAV particle comprises a viral genome comprising SEQ ID NO: 2271 and an AAVPHP.B capsid. In some embodiments, the AAV particle comprises a viral genome comprising SEQ ID NO: 2271 and an AAVPHP.N capsid. In some embodiments, the AAV particle comprises a viral genome comprising SEQ ID NO: 2271 and an AAV2 variant capsid. In some embodiments, the AAV particle comprises a viral genome comprising SEQ ID NO: 2271 and an AAV2/3 variant capsid. In some embodiments, the AAV particle comprises a viral genome comprising SEQ ID NO: 2271 and a capsid comprising SEQ ID NO: 1. In some embodiments, the AAV particle comprises a viral genome comprising SEQ ID NO: 2271 and a capsid encoded by a nucleic acid sequence comprising SEQ ID NO: 2. In some embodiments, the AAV particle comprises a viral genome comprising SEQ ID NO: 2271 and a capsid comprising SEQ ID NO: 4534. In some embodiments, the AAV particle comprises a viral genome comprising SEQ ID NO: 2271 and a capsid encoded by a nucleic acid sequence comprising SEQ ID NO: 3. In some embodiments, the AAV particle comprises a viral genome comprising SEQ ID NO: 2271 and a capsid comprising SEQ ID NO: 138. In some embodiments, the AAV particle comprises a viral genome comprising SEQ ID NO: 2271 and a capsid encoded by a nucleic acid sequence comprising SEQ ID NO: 137. In some embodiments, the AAV particle comprises a viral genome comprising SEQ ID NO: 2271 and a capsid comprising SEQ ID NO: 5. In some embodiments, the AAV particle comprises a viral genome comprising SEQ ID NO: 2271 and a capsid encoded by a nucleic acid sequence comprising SEQ ID NO: 6. In some embodiments, the AAV particle comprises a viral genome comprising SEQ ID NO: 2271 and a capsid comprising SEQ ID NO: 4. In some embodiments, the AAV particle comprises a viral genome comprising SEQ ID NO: 2271 and a capsid comprising SEQ ID NO: 11. In some embodiments, the AAV particle comprises a viral genome comprising SEQ ID NO: 2271 and a capsid comprising SEQ ID NO: 2679. In some embodiments, the AAV particle comprises a viral genome comprising SEQ ID NO: 2271 and a capsid comprising SEQ ID NO: 2809. In some embodiments, the AAV particle comprises a viral genome comprising SEQ ID NO: 2271 and a capsid comprising SEQ ID NO: 2871.

In some embodiments, the AAV particle comprises a viral genome comprising SEQ ID NO: 2272 and a VOY101 capsid. In some embodiments, the AAV particle comprises a viral genome comprising SEQ ID NO: 2272 and a VOY201 capsid. In some embodiments, the AAV particle comprises a viral genome comprising SEQ ID NO: 2272 and an AAV9 capsid. In some embodiments, the AAV particle comprises a viral genome comprising SEQ ID NO: 2272 and an AAV9K449R capsid. In some embodiments, the AAV particle comprises a viral genome comprising SEQ ID NO: 2272 and an AAVPHP.B capsid. In some embodiments, the AAV particle comprises a viral genome comprising SEQ ID NO: 2272 and an AAVPHP.N capsid. In some embodiments, the AAV particle comprises a viral genome comprising SEQ ID NO: 2272 and an AAV2 variant capsid. In some embodiments, the AAV particle comprises a viral genome comprising SEQ ID NO: 2272 and an AAV2/3 variant capsid. In some embodiments, the AAV particle comprises a viral genome comprising SEQ ID NO: 2272 and a capsid comprising SEQ ID NO: 1. In some embodiments, the AAV particle comprises a viral genome comprising SEQ ID NO: 2272 and a capsid encoded by a nucleic acid sequence comprising SEQ ID NO: 2. In some embodiments, the AAV particle comprises a viral genome comprising SEQ ID NO: 2272 and a capsid comprising SEQ ID NO: 4534. In some embodiments, the AAV particle comprises a viral genome comprising SEQ ID NO: 2272 and a capsid encoded by a nucleic acid sequence comprising SEQ ID NO: 3. In some embodiments, the AAV particle comprises a viral genome comprising SEQ ID NO: 2272 and a capsid comprising SEQ ID NO: 138. In some embodiments, the AAV particle comprises a viral genome comprising SEQ ID NO: 2272 and a capsid encoded by a nucleic acid sequence comprising SEQ ID NO: 137. In some embodiments, the AAV particle comprises a viral genome comprising SEQ ID NO: 2272 and a capsid comprising SEQ ID NO: 5. In some embodiments, the AAV particle comprises a viral genome comprising SEQ ID NO: 2272 and a capsid encoded by a nucleic acid sequence comprising SEQ ID NO: 6. In some embodiments, the AAV particle comprises a viral genome comprising SEQ ID NO: 2272 and a capsid comprising SEQ ID NO: 4. In some embodiments, the AAV particle comprises a viral genome comprising SEQ ID NO: 2272 and a capsid comprising SEQ ID NO: 11. In some embodiments, the AAV particle comprises a viral genome comprising SEQ ID NO: 2272 and a capsid comprising SEQ ID NO: 2679. In some embodiments, the AAV particle comprises a viral genome comprising SEQ ID NO: 2272 and a capsid comprising SEQ ID NO: 2809. In some embodiments, the AAV particle comprises a viral genome comprising SEQ ID NO: 2272 and a capsid comprising SEQ ID NO: 2871.

In some embodiments, the AAV particle comprises a viral genome comprising SEQ ID NO: 2273 and a VOY101 capsid. In some embodiments, the AAV particle comprises a viral genome comprising SEQ ID NO: 2273 and a VOY201 capsid. In some embodiments, the AAV particle comprises a viral genome comprising SEQ ID NO: 2273 and an AAV9 capsid. In some embodiments, the AAV particle comprises a viral genome comprising SEQ ID NO: 2273 and an AAV9K449R capsid. In some embodiments, the AAV particle comprises a viral genome comprising SEQ ID NO: 2273 and an AAVPHP.B capsid. In some embodiments, the AAV particle comprises a viral genome comprising SEQ ID NO: 2273 and an AAVPHP.N capsid. In some embodiments, the AAV particle comprises a viral genome comprising SEQ ID NO: 2273 and an AAV2 variant capsid. In some embodiments, the AAV particle comprises a viral genome comprising SEQ ID NO: 2273 and an AAV2/3 variant capsid. In some embodiments, the AAV particle comprises a viral genome comprising SEQ ID NO: 2273 and a capsid comprising SEQ ID NO: 1. In some embodiments, the AAV particle comprises a viral genome comprising SEQ ID NO: 2273 and a capsid encoded by a nucleic acid sequence comprising SEQ ID NO: 2. In some embodiments, the AAV particle comprises a viral genome comprising SEQ ID NO: 2273 and a capsid comprising SEQ ID NO: 4534. In some embodiments, the AAV particle comprises a viral genome comprising SEQ ID NO: 2273 and a capsid encoded by a nucleic acid sequence comprising SEQ ID NO: 3. In some embodiments, the AAV particle comprises a viral genome comprising SEQ ID NO: 2273 and a capsid comprising SEQ ID NO: 138. In some embodiments, the AAV particle comprises a viral genome comprising SEQ ID NO: 2273 and a capsid encoded by a nucleic acid sequence comprising SEQ ID NO: 137. In some embodiments, the AAV particle comprises a viral genome comprising SEQ ID NO: 2273 and a capsid comprising SEQ ID NO: 5. In some embodiments, the AAV particle comprises a viral genome comprising SEQ ID NO: 2273 and a capsid encoded by a nucleic acid sequence comprising SEQ ID NO: 6. In some embodiments, the AAV particle comprises a viral genome comprising SEQ ID NO: 2273 and a capsid comprising SEQ ID NO: 4. In some embodiments, the AAV particle comprises a viral genome comprising SEQ ID NO: 2273 and a capsid comprising SEQ ID NO: 11. In some embodiments, the AAV particle comprises a viral genome comprising SEQ ID NO: 2273 and a capsid comprising SEQ ID NO: 2679. In some embodiments, the AAV particle comprises a viral genome comprising SEQ ID NO: 2273 and a capsid comprising SEQ ID NO: 2809. In some embodiments, the AAV particle comprises a viral genome comprising SEQ ID NO: 2273 and a capsid comprising SEQ ID NO: 2871.

In some embodiments, the AAV particle comprises a viral genome comprising SEQ ID NO: 2274 and a VOY101 capsid. In some embodiments, the AAV particle comprises a viral genome comprising SEQ ID NO: 2274 and a VOY201 capsid. In some embodiments, the AAV particle comprises a viral genome comprising SEQ ID NO: 2274 and an AAV9 capsid. In some embodiments, the AAV particle comprises a viral genome comprising SEQ ID NO: 2274 and an AAV9K449R capsid. In some embodiments, the AAV particle comprises a viral genome comprising SEQ ID NO: 2274 and an AAVPHP.B capsid. In some embodiments, the AAV particle comprises a viral genome comprising SEQ ID NO: 2274 and an AAVPHP.N capsid. In some embodiments, the AAV particle comprises a viral genome comprising SEQ ID NO: 2274 and an AAV2 variant capsid. In some embodiments, the AAV particle comprises a viral genome comprising SEQ ID NO: 2274 and an AAV2/3 variant capsid. In some embodiments, the AAV particle comprises a viral genome comprising SEQ ID NO: 2274 and a capsid comprising SEQ ID NO: 1. In some embodiments, the AAV particle comprises a viral genome comprising SEQ ID NO: 2274 and a capsid encoded by a nucleic acid sequence comprising SEQ ID NO: 2. In some embodiments, the AAV particle comprises a viral genome comprising SEQ ID NO: 2274 and a capsid comprising SEQ ID NO: 4534. In some embodiments, the AAV particle comprises a viral genome comprising SEQ ID NO: 2274 and a capsid encoded by a nucleic acid sequence comprising SEQ ID NO: 3. In some embodiments, the AAV particle comprises a viral genome comprising SEQ ID NO: 2274 and a capsid comprising SEQ ID NO: 138. In some embodiments, the AAV particle comprises a viral genome comprising SEQ ID NO: 2274 and a capsid encoded by a nucleic acid sequence comprising SEQ ID NO: 137. In some embodiments, the AAV particle comprises a viral genome comprising SEQ ID NO: 2274 and a capsid comprising SEQ ID NO: 5. In some embodiments, the AAV particle comprises a viral genome comprising SEQ ID NO: 2274 and a capsid encoded by a nucleic acid sequence comprising SEQ ID NO: 6. In some embodiments, the AAV particle comprises a viral genome comprising SEQ ID NO: 2274 and a capsid comprising SEQ ID NO: 4. In some embodiments, the AAV particle comprises a viral genome comprising SEQ ID NO: 2274 and a capsid comprising SEQ ID NO: 11. In some embodiments, the AAV particle comprises a viral genome comprising SEQ ID NO: 2274 and a capsid comprising SEQ ID NO: 2679. In some embodiments, the AAV particle comprises a viral genome comprising SEQ ID NO: 2274 and a capsid comprising SEQ ID NO: 2809. In some embodiments, the AAV particle comprises a viral genome comprising SEQ ID NO: 2274 and a capsid comprising SEQ ID NO: 2871.

In some embodiments, the AAV particle comprises a viral genome comprising SEQ ID NO: 2275 and a VOY101 capsid. In some embodiments, the AAV particle comprises a viral genome comprising SEQ ID NO: 2275 and a VOY201 capsid. In some embodiments, the AAV particle comprises a viral genome comprising SEQ ID NO: 2275 and an AAV9 capsid. In some embodiments, the AAV particle comprises a viral genome comprising SEQ ID NO: 2275 and an AAV9K449R capsid. In some embodiments, the AAV particle comprises a viral genome comprising SEQ ID NO: 2275 and an AAVPHP.B capsid. In some embodiments, the AAV particle comprises a viral genome comprising SEQ ID NO: 2275 and an AAVPHP.N capsid. In some embodiments, the AAV particle comprises a viral genome comprising SEQ ID NO: 2275 and an AAV2 variant capsid. In some embodiments, the AAV particle comprises a viral genome comprising SEQ ID NO: 2275 and an AAV2/3 variant capsid. In some embodiments, the AAV particle comprises a viral genome comprising SEQ ID NO: 2275 and a capsid comprising SEQ ID NO: 1. In some embodiments, the AAV particle comprises a viral genome comprising SEQ ID NO: 2275 and a capsid encoded by a nucleic acid sequence comprising SEQ ID NO: 2. In some embodiments, the AAV particle comprises a viral genome comprising SEQ ID NO: 2275 and a capsid comprising SEQ ID NO: 4534. In some embodiments, the AAV particle comprises a viral genome comprising SEQ ID NO: 2275 and a capsid encoded by a nucleic acid sequence comprising SEQ ID NO: 3. In some embodiments, the AAV particle comprises a viral genome comprising SEQ ID NO: 2275 and a capsid comprising SEQ ID NO: 138. In some embodiments, the AAV particle comprises a viral genome comprising SEQ ID NO: 2275 and a capsid encoded by a nucleic acid sequence comprising SEQ ID NO: 137. In some embodiments, the AAV particle comprises a viral genome comprising SEQ ID NO: 2275 and a capsid comprising SEQ ID NO: 5. In some embodiments, the AAV particle comprises a viral genome comprising SEQ ID NO: 2275 and a capsid encoded by a nucleic acid sequence comprising SEQ ID NO: 6. In some embodiments, the AAV particle comprises a viral genome comprising SEQ ID NO: 2275 and a capsid comprising SEQ ID NO: 4. In some embodiments, the AAV particle comprises a viral genome comprising SEQ ID NO: 2275 and a capsid comprising SEQ ID NO: 11. In some embodiments, the AAV particle comprises a viral genome comprising SEQ ID NO: 2275 and a capsid comprising SEQ ID NO: 2679. In some embodiments, the AAV particle comprises a viral genome comprising SEQ ID NO: 2275 and a capsid comprising SEQ ID NO: 2809. In some embodiments, the AAV particle comprises a viral genome comprising SEQ ID NO: 2275 and a capsid comprising SEQ ID NO: 2871.

In some embodiments, the AAV particle comprises a viral genome comprising SEQ ID NO: 2276 and a VOY101 capsid. In some embodiments, the AAV particle comprises a viral genome comprising SEQ ID NO: 2276 and a VOY201 capsid. In some embodiments, the AAV particle comprises a viral genome comprising SEQ ID NO: 2276 and an AAV9 capsid. In some embodiments, the AAV particle comprises a viral genome comprising SEQ ID NO: 2276 and an AAV9K449R capsid. In some embodiments, the AAV particle comprises a viral genome comprising SEQ ID NO: 2276 and an AAVPHP.B capsid. In some embodiments, the AAV particle comprises a viral genome comprising SEQ ID NO: 2276 and an AAVPHP.N capsid. In some embodiments, the AAV particle comprises a viral genome comprising SEQ ID NO: 2276 and an AAV2 variant capsid. In some embodiments, the AAV particle comprises a viral genome comprising SEQ ID NO: 2276 and an AAV2/3 variant capsid. In some embodiments, the AAV particle comprises a viral genome comprising SEQ ID NO: 2276 and a capsid comprising SEQ ID NO: 1. In some embodiments, the AAV particle comprises a viral genome comprising SEQ ID NO: 2276 and a capsid encoded by a nucleic acid sequence comprising SEQ ID NO: 2. In some embodiments, the AAV particle comprises a viral genome comprising SEQ ID NO: 2276 and a capsid comprising SEQ ID NO: 4534. In some embodiments, the AAV particle comprises a viral genome comprising SEQ ID NO: 2276 and a capsid encoded by a nucleic acid sequence comprising SEQ ID NO: 3. In some embodiments, the AAV particle comprises a viral genome comprising SEQ ID NO: 2276 and a capsid comprising SEQ ID NO: 138. In some embodiments, the AAV particle comprises a viral genome comprising SEQ ID NO: 2276 and a capsid encoded by a nucleic acid sequence comprising SEQ ID NO: 137. In some embodiments, the AAV particle comprises a viral genome comprising SEQ ID NO: 2276 and a capsid comprising SEQ ID NO: 5. In some embodiments, the AAV particle comprises a viral genome comprising SEQ ID NO: 2276 and a capsid encoded by a nucleic acid sequence comprising SEQ ID NO: 6. In some embodiments, the AAV particle comprises a viral genome comprising SEQ ID NO: 2276 and a capsid comprising SEQ ID NO: 4. In some embodiments, the AAV particle comprises a viral genome comprising SEQ ID NO: 2276 and a capsid comprising SEQ ID NO: 11. In some embodiments, the AAV particle comprises a viral genome comprising SEQ ID NO: 2276 and a capsid comprising SEQ ID NO: 2679. In some embodiments, the AAV particle comprises a viral genome comprising SEQ ID NO: 2276 and a capsid comprising SEQ ID NO: 2809. In some embodiments, the AAV particle comprises a viral genome comprising SEQ ID NO: 2276 and a capsid comprising SEQ ID NO: 2871.

In some embodiments, the AAV particle comprises a viral genome comprising SEQ ID NO: 2277 and a VOY101 capsid. In some embodiments, the AAV particle comprises a viral genome comprising SEQ ID NO: 2277 and a VOY201 capsid. In some embodiments, the AAV particle comprises a viral genome comprising SEQ ID NO: 2277 and an AAV9 capsid. In some embodiments, the AAV particle comprises a viral genome comprising SEQ ID NO: 2277 and an AAV9K449R capsid. In some embodiments, the AAV particle comprises a viral genome comprising SEQ ID NO: 2277 and an AAVPHP.B capsid. In some embodiments, the AAV particle comprises a viral genome comprising SEQ ID NO: 2277 and an AAVPHP.N capsid. In some embodiments, the AAV particle comprises a viral genome comprising SEQ ID NO: 2277 and an AAV2 variant capsid. In some embodiments, the AAV particle comprises a viral genome comprising SEQ ID NO: 2277 and an AAV2/3 variant capsid. In some embodiments, the AAV particle comprises a viral genome comprising SEQ ID NO: 2277 and a capsid comprising SEQ ID NO: 1. In some embodiments, the AAV particle comprises a viral genome comprising SEQ ID NO: 2277 and a capsid encoded by a nucleic acid sequence comprising SEQ ID NO: 2. In some embodiments, the AAV particle comprises a viral genome comprising SEQ ID NO: 2277 and a capsid comprising SEQ ID NO: 4534. In some embodiments, the AAV particle comprises a viral genome comprising SEQ ID NO: 2277 and a capsid encoded by a nucleic acid sequence comprising SEQ ID NO: 3. In some embodiments, the AAV particle comprises a viral genome comprising SEQ ID NO: 2277 and a capsid comprising SEQ ID NO: 138. In some embodiments, the AAV particle comprises a viral genome comprising SEQ ID NO: 2277 and a capsid encoded by a nucleic acid sequence comprising SEQ ID NO: 137. In some embodiments, the AAV particle comprises a viral genome comprising SEQ ID NO: 2277 and a capsid comprising SEQ ID NO: 5. In some embodiments, the AAV particle comprises a viral genome comprising SEQ ID NO: 2277 and a capsid encoded by a nucleic acid sequence comprising SEQ ID NO: 6. In some embodiments, the AAV particle comprises a viral genome comprising SEQ ID NO: 2277 and a capsid comprising SEQ ID NO: 4. In some embodiments, the AAV particle comprises a viral genome comprising SEQ ID NO: 2277 and a capsid comprising SEQ ID NO: 11. In some embodiments, the AAV particle comprises a viral genome comprising SEQ ID NO: 2277 and a capsid comprising SEQ ID NO: 2679. In some embodiments, the AAV particle comprises a viral genome comprising SEQ ID NO: 2277 and a capsid comprising SEQ ID NO: 2809. In some embodiments, the AAV particle comprises a viral genome comprising SEQ ID NO: 2277 and a capsid comprising SEQ ID NO: 2871.

In some embodiments, the AAV particle comprises a viral genome comprising SEQ ID NO: 2278 and a VOY101 capsid. In some embodiments, the AAV particle comprises a viral genome comprising SEQ ID NO: 2278 and a VOY201 capsid. In some embodiments, the AAV particle comprises a viral genome comprising SEQ ID NO: 2278 and an AAV9 capsid. In some embodiments, the AAV particle comprises a viral genome comprising SEQ ID NO: 2278 and an AAV9K449R capsid. In some embodiments, the AAV particle comprises a viral genome comprising SEQ ID NO: 2278 and an AAVPHP.B capsid. In some embodiments, the AAV particle comprises a viral genome comprising SEQ ID NO: 2278 and an AAVPHP.N capsid. In some embodiments, the AAV particle comprises a viral genome comprising SEQ ID NO: 2278 and an AAV2 variant capsid. In some embodiments, the AAV particle comprises a viral genome comprising SEQ ID NO: 2278 and an AAV2/3 variant capsid. In some embodiments, the AAV particle comprises a viral genome comprising SEQ ID NO: 2278 and a capsid comprising SEQ ID NO: 1. In some embodiments, the AAV particle comprises a viral genome comprising SEQ ID NO: 2278 and a capsid encoded by a nucleic acid sequence comprising SEQ ID NO: 2. In some embodiments, the AAV particle comprises a viral genome comprising SEQ ID NO: 2278 and a capsid comprising SEQ ID NO: 4534. In some embodiments, the AAV particle comprises a viral genome comprising SEQ ID NO: 2278 and a capsid encoded by a nucleic acid sequence comprising SEQ ID NO: 3. In some embodiments, the AAV particle comprises a viral genome comprising SEQ ID NO: 2278 and a capsid comprising SEQ ID NO: 138. In some embodiments, the AAV particle comprises a viral genome comprising SEQ ID NO: 2278 and a capsid encoded by a nucleic acid sequence comprising SEQ ID NO: 137. In some embodiments, the AAV particle comprises a viral genome comprising SEQ ID NO: 2278 and a capsid comprising SEQ ID NO: 5. In some embodiments, the AAV particle comprises a viral genome comprising SEQ ID NO: 2278 and a capsid encoded by a nucleic acid sequence comprising SEQ ID NO: 6. In some embodiments, the AAV particle comprises a viral genome comprising SEQ ID NO: 2278 and a capsid comprising SEQ ID NO: 4. In some embodiments, the AAV particle comprises a viral genome comprising SEQ ID NO: 2278 and a capsid comprising SEQ ID NO: 11. In some embodiments, the AAV particle comprises a viral genome comprising SEQ ID NO: 2278 and a capsid comprising SEQ ID NO: 2679. In some embodiments, the AAV particle comprises a viral genome comprising SEQ ID NO: 2278 and a capsid comprising SEQ ID NO: 2809. In some embodiments, the AAV particle comprises a viral genome comprising SEQ ID NO: 2278 and a capsid comprising SEQ ID NO: 2871.

In some embodiments, the AAV particle comprises a viral genome comprising SEQ ID NO: 2279 and a VOY101 capsid. In some embodiments, the AAV particle comprises a viral genome comprising SEQ ID NO: 2279 and a VOY201 capsid. In some embodiments, the AAV particle comprises a viral genome comprising SEQ ID NO: 2279 and an AAV9 capsid. In some embodiments, the AAV particle comprises a viral genome comprising SEQ ID NO: 2279 and an AAV9K449R capsid. In some embodiments, the AAV particle comprises a viral genome comprising SEQ ID NO: 2279 and an AAVPHP.B capsid. In some embodiments, the AAV particle comprises a viral genome comprising SEQ ID NO: 2279 and an AAVPHP.N capsid. In some embodiments, the AAV particle comprises a viral genome comprising SEQ ID NO: 2279 and an AAV2 variant capsid. In some embodiments, the AAV particle comprises a viral genome comprising SEQ ID NO: 2279 and an AAV2/3 variant capsid. In some embodiments, the AAV particle comprises a viral genome comprising SEQ ID NO: 2279 and a capsid comprising SEQ ID NO: 1. In some embodiments, the AAV particle comprises a viral genome comprising SEQ ID NO: 2279 and a capsid encoded by a nucleic acid sequence comprising SEQ ID NO: 2. In some embodiments, the AAV particle comprises a viral genome comprising SEQ ID NO: 2279 and a capsid comprising SEQ ID NO: 4534. In some embodiments, the AAV particle comprises a viral genome comprising SEQ ID NO: 2279 and a capsid encoded by a nucleic acid sequence comprising SEQ ID NO: 3. In some embodiments, the AAV particle comprises a viral genome comprising SEQ ID NO: 2279 and a capsid comprising SEQ ID NO: 138. In some embodiments, the AAV particle comprises a viral genome comprising SEQ ID NO: 2279 and a capsid encoded by a nucleic acid sequence comprising SEQ ID NO: 137. In some embodiments, the AAV particle comprises a viral genome comprising SEQ ID NO: 2279 and a capsid comprising SEQ ID NO: 5. In some embodiments, the AAV particle comprises a viral genome comprising SEQ ID NO: 2279 and a capsid encoded by a nucleic acid sequence comprising SEQ ID NO: 6. In some embodiments, the AAV particle comprises a viral genome comprising SEQ ID NO: 2279 and a capsid comprising SEQ ID NO: 4. In some embodiments, the AAV particle comprises a viral genome comprising SEQ ID NO: 2279 and a capsid comprising SEQ ID NO: 11. In some embodiments, the AAV particle comprises a viral genome comprising SEQ ID NO: 2279 and a capsid comprising SEQ ID NO: 2679. In some embodiments, the AAV particle comprises a viral genome comprising SEQ ID NO: 2279 and a capsid comprising SEQ ID NO: 2809. In some embodiments, the AAV particle comprises a viral genome comprising SEQ ID NO: 2279 and a capsid comprising SEQ ID NO: 2871.

In some embodiments, the AAV particle comprises a viral genome comprising SEQ ID NO: 2280 and a VOY101 capsid. In some embodiments, the AAV particle comprises a viral genome comprising SEQ ID NO: 2280 and a VOY201 capsid. In some embodiments, the AAV particle comprises a viral genome comprising SEQ ID NO: 2280 and an AAV9 capsid. In some embodiments, the AAV particle comprises a viral genome comprising SEQ ID NO: 2280 and an AAV9K449R capsid. In some embodiments, the AAV particle comprises a viral genome comprising SEQ ID NO: 2280 and an AAVPHP.B capsid. In some embodiments, the AAV particle comprises a viral genome comprising SEQ ID NO: 2280 and an AAVPHP.N capsid. In some embodiments, the AAV particle comprises a viral genome comprising SEQ ID NO: 2280 and an AAV2 variant capsid. In some embodiments, the AAV particle comprises a viral genome comprising SEQ ID NO: 2280 and an AAV2/3 variant capsid. In some embodiments, the AAV particle comprises a viral genome comprising SEQ ID NO: 2280 and a capsid comprising SEQ ID NO: 1. In some embodiments, the AAV particle comprises a viral genome comprising SEQ ID NO: 2280 and a capsid encoded by a nucleic acid sequence comprising SEQ ID NO: 2. In some embodiments, the AAV particle comprises a viral genome comprising SEQ ID NO: 2280 and a capsid comprising SEQ ID NO: 4534. In some embodiments, the AAV particle comprises a viral genome comprising SEQ ID NO: 2280 and a capsid encoded by a nucleic acid sequence comprising SEQ ID NO: 3. In some embodiments, the AAV particle comprises a viral genome comprising SEQ ID NO: 2280 and a capsid comprising SEQ ID NO: 138. In some embodiments, the AAV particle comprises a viral genome comprising SEQ ID NO: 2280 and a capsid encoded by a nucleic acid sequence comprising SEQ ID NO: 137. In some embodiments, the AAV particle comprises a viral genome comprising SEQ ID NO: 2280 and a capsid comprising SEQ ID NO: 5. In some embodiments, the AAV particle comprises a viral genome comprising SEQ ID NO: 2280 and a capsid encoded by a nucleic acid sequence comprising SEQ ID NO: 6. In some embodiments, the AAV particle comprises a viral genome comprising SEQ ID NO: 2280 and a capsid comprising SEQ ID NO: 4. In some embodiments, the AAV particle comprises a viral genome comprising SEQ ID NO: 2280 and a capsid comprising SEQ ID NO: 11. In some embodiments, the AAV particle comprises a viral genome comprising SEQ ID NO: 2280 and a capsid comprising SEQ ID NO: 2679. In some embodiments, the AAV particle comprises a viral genome comprising SEQ ID NO: 2280 and a capsid comprising SEQ ID NO: 2809. In some embodiments, the AAV particle comprises a viral genome comprising SEQ ID NO: 2280 and a capsid comprising SEQ ID NO: 2871.

In some embodiments, the AAV particle comprises a viral genome comprising SEQ ID NO: 2281 and a VOY101 capsid. In some embodiments, the AAV particle comprises a viral genome comprising SEQ ID NO: 2281 and a VOY201 capsid. In some embodiments, the AAV particle comprises a viral genome comprising SEQ ID NO: 2281 and an AAV9 capsid. In some embodiments, the AAV particle comprises a viral genome comprising SEQ ID NO: 2281 and an AAV9K449R capsid. In some embodiments, the AAV particle comprises a viral genome comprising SEQ ID NO: 2281 and an AAVPHP.B capsid. In some embodiments, the AAV particle comprises a viral genome comprising SEQ ID NO: 2281 and an AAVPHP.N capsid. In some embodiments, the AAV particle comprises a viral genome comprising SEQ ID NO: 2281 and an AAV2 variant capsid. In some embodiments, the AAV particle comprises a viral genome comprising SEQ ID NO: 2281 and an AAV2/3 variant capsid. In some embodiments, the AAV particle comprises a viral genome comprising SEQ ID NO: 2281 and a capsid comprising SEQ ID NO: 1. In some embodiments, the AAV particle comprises a viral genome comprising SEQ ID NO: 2281 and a capsid encoded by a nucleic acid sequence comprising SEQ ID NO: 2. In some embodiments, the AAV particle comprises a viral genome comprising SEQ ID NO: 2281 and a capsid comprising SEQ ID NO: 4534. In some embodiments, the AAV particle comprises a viral genome comprising SEQ ID NO: 2281 and a capsid encoded by a nucleic acid sequence comprising SEQ ID NO: 3. In some embodiments, the AAV particle comprises a viral genome comprising SEQ ID NO: 2281 and a capsid comprising SEQ ID NO: 138. In some embodiments, the AAV particle comprises a viral genome comprising SEQ ID NO: 2281 and a capsid encoded by a nucleic acid sequence comprising SEQ ID NO: 137. In some embodiments, the AAV particle comprises a viral genome comprising SEQ ID NO: 2281 and a capsid comprising SEQ ID NO: 5. In some embodiments, the AAV particle comprises a viral genome comprising SEQ ID NO: 2281 and a capsid encoded by a nucleic acid sequence comprising SEQ ID NO: 6. In some embodiments, the AAV particle comprises a viral genome comprising SEQ ID NO: 2281 and a capsid comprising SEQ ID NO: 4. In some embodiments, the AAV particle comprises a viral genome comprising SEQ ID NO: 2281 and a capsid comprising SEQ ID NO: 11. In some embodiments, the AAV particle comprises a viral genome comprising SEQ ID NO: 2281 and a capsid comprising SEQ ID NO: 2679. In some embodiments, the AAV particle comprises a viral genome comprising SEQ ID NO: 2281 and a capsid comprising SEQ ID NO: 2809. In some embodiments, the AAV particle comprises a viral genome comprising SEQ ID NO: 2281 and a capsid comprising SEQ ID NO: 2871.

In some embodiments, the AAV particle comprises a viral genome comprising SEQ ID NO: 2282 and a VOY101 capsid. In some embodiments, the AAV particle comprises a viral genome comprising SEQ ID NO: 2282 and a VOY201 capsid. In some embodiments, the AAV particle comprises a viral genome comprising SEQ ID NO: 2282 and an AAV9 capsid. In some embodiments, the AAV particle comprises a viral genome comprising SEQ ID NO: 2282 and an AAV9K449R capsid. In some embodiments, the AAV particle comprises a viral genome comprising SEQ ID NO: 2282 and an AAVPHP.B capsid. In some embodiments, the AAV particle comprises a viral genome comprising SEQ ID NO: 2282 and an AAVPHP.N capsid. In some embodiments, the AAV particle comprises a viral genome comprising SEQ ID NO: 2282 and an AAV2 variant capsid. In some embodiments, the AAV particle comprises a viral genome comprising SEQ ID NO: 2282 and an AAV2/3 variant capsid. In some embodiments, the AAV particle comprises a viral genome comprising SEQ ID NO: 2282 and a capsid comprising SEQ ID NO: 1. In some embodiments, the AAV particle comprises a viral genome comprising SEQ ID NO: 2282 and a capsid encoded by a nucleic acid sequence comprising SEQ ID NO: 2. In some embodiments, the AAV particle comprises a viral genome comprising SEQ ID NO: 2282 and a capsid comprising SEQ ID NO: 4534. In some embodiments, the AAV particle comprises a viral genome comprising SEQ ID NO: 2282 and a capsid encoded by a nucleic acid sequence comprising SEQ ID NO: 3. In some embodiments, the AAV particle comprises a viral genome comprising SEQ ID NO: 2282 and a capsid comprising SEQ ID NO: 138. In some embodiments, the AAV particle comprises a viral genome comprising SEQ ID NO: 2282 and a capsid encoded by a nucleic acid sequence comprising SEQ ID NO: 137. In some embodiments, the AAV particle comprises a viral genome comprising SEQ ID NO: 2282 and a capsid comprising SEQ ID NO: 5. In some embodiments, the AAV particle comprises a viral genome comprising SEQ ID NO: 2282 and a capsid encoded by a nucleic acid sequence comprising SEQ ID NO: 6. In some embodiments, the AAV particle comprises a viral genome comprising SEQ ID NO: 2282 and a capsid comprising SEQ ID NO: 4. In some embodiments, the AAV particle comprises a viral genome comprising SEQ ID NO: 2282 and a capsid comprising SEQ ID NO: 11. In some embodiments, the AAV particle comprises a viral genome comprising SEQ ID NO: 2282 and a capsid comprising SEQ ID NO: 2679. In some embodiments, the AAV particle comprises a viral genome comprising SEQ ID NO: 2282 and a capsid comprising SEQ ID NO: 2809. In some embodiments, the AAV particle comprises a viral genome comprising SEQ ID NO: 2282 and a capsid comprising SEQ ID NO: 2871.

In some embodiments, the AAV particle comprises a viral genome comprising SEQ ID NO: 2283 and a VOY101 capsid. In some embodiments, the AAV particle comprises a viral genome comprising SEQ ID NO: 2283 and a VOY201 capsid. In some embodiments, the AAV particle comprises a viral genome comprising SEQ ID NO: 2283 and an AAV9 capsid. In some embodiments, the AAV particle comprises a viral genome comprising SEQ ID NO: 2283 and an AAV9K449R capsid. In some embodiments, the AAV particle comprises a viral genome comprising SEQ ID NO: 2283 and an AAVPHP.B capsid. In some embodiments, the AAV particle comprises a viral genome comprising SEQ ID NO: 2283 and an AAVPHP.N capsid. In some embodiments, the AAV particle comprises a viral genome comprising SEQ ID NO: 2283 and an AAV2 variant capsid. In some embodiments, the AAV particle comprises a viral genome comprising SEQ ID NO: 2283 and an AAV2/3 variant capsid. In some embodiments, the AAV particle comprises a viral genome comprising SEQ ID NO: 2283 and a capsid comprising SEQ ID NO: 1. In some embodiments, the AAV particle comprises a viral genome comprising SEQ ID NO: 2283 and a capsid encoded by a nucleic acid sequence comprising SEQ ID NO: 2. In some embodiments, the AAV particle comprises a viral genome comprising SEQ ID NO: 2283 and a capsid comprising SEQ ID NO: 4534. In some embodiments, the AAV particle comprises a viral genome comprising SEQ ID NO: 2283 and a capsid encoded by a nucleic acid sequence comprising SEQ ID NO: 3. In some embodiments, the AAV particle comprises a viral genome comprising SEQ ID NO: 2283 and a capsid comprising SEQ ID NO: 138. In some embodiments, the AAV particle comprises a viral genome comprising SEQ ID NO: 2283 and a capsid encoded by a nucleic acid sequence comprising SEQ ID NO: 137. In some embodiments, the AAV particle comprises a viral genome comprising SEQ ID NO: 2283 and a capsid comprising SEQ ID NO: 5. In some embodiments, the AAV particle comprises a viral genome comprising SEQ ID NO: 2283 and a capsid encoded by a nucleic acid sequence comprising SEQ ID NO: 6. In some embodiments, the AAV particle comprises a viral genome comprising SEQ ID NO: 2283 and a capsid comprising SEQ ID NO: 4. In some embodiments, the AAV particle comprises a viral genome comprising SEQ ID NO: 2283 and a capsid comprising SEQ ID NO: 11. In some embodiments, the AAV particle comprises a viral genome comprising SEQ ID NO: 2283 and a capsid comprising SEQ ID NO: 2679. In some embodiments, the AAV particle comprises a viral genome comprising SEQ ID NO: 2283 and a capsid comprising SEQ ID NO: 2809. In some embodiments, the AAV particle comprises a viral genome comprising SEQ ID NO: 2283 and a capsid comprising SEQ ID NO: 2871.

In some embodiments, the AAV particle comprises a viral genome comprising SEQ ID NO: 2284 and a VOY101 capsid. In some embodiments, the AAV particle comprises a viral genome comprising SEQ ID NO: 2284 and a VOY201 capsid. In some embodiments, the AAV particle comprises a viral genome comprising SEQ ID NO: 2284 and an AAV9 capsid. In some embodiments, the AAV particle comprises a viral genome comprising SEQ ID NO: 2284 and an AAV9K449R capsid. In some embodiments, the AAV particle comprises a viral genome comprising SEQ ID NO: 2284 and an AAVPHP.B capsid. In some embodiments, the AAV particle comprises a viral genome comprising SEQ ID NO: 2284 and an AAVPHP.N capsid. In some embodiments, the AAV particle comprises a viral genome comprising SEQ ID NO: 2284 and an AAV2 variant capsid. In some embodiments, the AAV particle comprises a viral genome comprising SEQ ID NO: 2284 and an AAV2/3 variant capsid. In some embodiments, the AAV particle comprises a viral genome comprising SEQ ID NO: 2284 and a capsid comprising SEQ ID NO: 1. In some embodiments, the AAV particle comprises a viral genome comprising SEQ ID NO: 2284 and a capsid encoded by a nucleic acid sequence comprising SEQ ID NO: 2. In some embodiments, the AAV particle comprises a viral genome comprising SEQ ID NO: 2284 and a capsid comprising SEQ ID NO: 4534. In some embodiments, the AAV particle comprises a viral genome comprising SEQ ID NO: 2284 and a capsid encoded by a nucleic acid sequence comprising SEQ ID NO: 3. In some embodiments, the AAV particle comprises a viral genome comprising SEQ ID NO: 2284 and a capsid comprising SEQ ID NO: 138. In some embodiments, the AAV particle comprises a viral genome comprising SEQ ID NO: 2284 and a capsid encoded by a nucleic acid sequence comprising SEQ ID NO: 137. In some embodiments, the AAV particle comprises a viral genome comprising SEQ ID NO: 2284 and a capsid comprising SEQ ID NO: 5. In some embodiments, the AAV particle comprises a viral genome comprising SEQ ID NO: 2284 and a capsid encoded by a nucleic acid sequence comprising SEQ ID NO: 6. In some embodiments, the AAV particle comprises a viral genome comprising SEQ ID NO: 2284 and a capsid comprising SEQ ID NO: 4. In some embodiments, the AAV particle comprises a viral genome comprising SEQ ID NO: 2284 and a capsid comprising SEQ ID NO: 11. In some embodiments, the AAV particle comprises a viral genome comprising SEQ ID NO: 2284 and a capsid comprising SEQ ID NO: 2679. In some embodiments, the AAV particle comprises a viral genome comprising SEQ ID NO: 2284 and a capsid comprising SEQ ID NO: 2809. In some embodiments, the AAV particle comprises a viral genome comprising SEQ ID NO: 2284 and a capsid comprising SEQ ID NO: 2871.

In some embodiments, the AAV particle comprises a viral genome comprising SEQ ID NO: 2285 and a VOY101 capsid. In some embodiments, the AAV particle comprises a viral genome comprising SEQ ID NO: 2285 and a VOY201 capsid. In some embodiments, the AAV particle comprises a viral genome comprising SEQ ID NO: 2285 and an AAV9 capsid. In some embodiments, the AAV particle comprises a viral genome comprising SEQ ID NO: 2285 and an AAV9K449R capsid. In some embodiments, the AAV particle comprises a viral genome comprising SEQ ID NO: 2285 and an AAVPHP.B capsid. In some embodiments, the AAV particle comprises a viral genome comprising SEQ ID NO: 2285 and an AAVPHP.N capsid. In some embodiments, the AAV particle comprises a viral genome comprising SEQ ID NO: 2285 and an AAV2 variant capsid. In some embodiments, the AAV particle comprises a viral genome comprising SEQ ID NO: 2285 and an AAV2/3 variant capsid. In some embodiments, the AAV particle comprises a viral genome comprising SEQ ID NO: 2285 and a capsid comprising SEQ ID NO: 1. In some embodiments, the AAV particle comprises a viral genome comprising SEQ ID NO: 2285 and a capsid encoded by a nucleic acid sequence comprising SEQ ID NO: 2. In some embodiments, the AAV particle comprises a viral genome comprising SEQ ID NO: 2285 and a capsid comprising SEQ ID NO: 4534. In some embodiments, the AAV particle comprises a viral genome comprising SEQ ID NO: 2285 and a capsid encoded by a nucleic acid sequence comprising SEQ ID NO: 3. In some embodiments, the AAV particle comprises a viral genome comprising SEQ ID NO: 2285 and a capsid comprising SEQ ID NO: 138. In some embodiments, the AAV particle comprises a viral genome comprising SEQ ID NO: 2285 and a capsid encoded by a nucleic acid sequence comprising SEQ ID NO: 137. In some embodiments, the AAV particle comprises a viral genome comprising SEQ ID NO: 2285 and a capsid comprising SEQ ID NO: 5. In some embodiments, the AAV particle comprises a viral genome comprising SEQ ID NO: 2285 and a capsid encoded by a nucleic acid sequence comprising SEQ ID NO: 6. In some embodiments, the AAV particle comprises a viral genome comprising SEQ ID NO: 2285 and a capsid comprising SEQ ID NO: 4. In some embodiments, the AAV particle comprises a viral genome comprising SEQ ID NO: 2285 and a capsid comprising SEQ ID NO: 11. In some embodiments, the AAV particle comprises a viral genome comprising SEQ ID NO: 2285 and a capsid comprising SEQ ID NO: 2679. In some embodiments, the AAV particle comprises a viral genome comprising SEQ ID NO: 2285 and a capsid comprising SEQ ID NO: 2809. In some embodiments, the AAV particle comprises a viral genome comprising SEQ ID NO: 2285 and a capsid comprising SEQ ID NO: 2871.

In some embodiments, the AAV particle comprises a viral genome comprising SEQ ID NO: 2286 and a VOY101 capsid. In some embodiments, the AAV particle comprises a viral genome comprising SEQ ID NO: 2286 and a VOY201 capsid. In some embodiments, the AAV particle comprises a viral genome comprising SEQ ID NO: 2286 and an AAV9 capsid. In some embodiments, the AAV particle comprises a viral genome comprising SEQ ID NO: 2286 and an AAV9K449R capsid. In some embodiments, the AAV particle comprises a viral genome comprising SEQ ID NO: 2286 and an AAVPHP.B capsid. In some embodiments, the AAV particle comprises a viral genome comprising SEQ ID NO: 2286 and an AAVPHP.N capsid. In some embodiments, the AAV particle comprises a viral genome comprising SEQ ID NO: 2286 and an AAV2 variant capsid. In some embodiments, the AAV particle comprises a viral genome comprising SEQ ID NO: 2286 and an AAV2/3 variant capsid. In some embodiments, the AAV particle comprises a viral genome comprising SEQ ID NO: 2286 and a capsid comprising SEQ ID NO: 1. In some embodiments, the AAV particle comprises a viral genome comprising SEQ ID NO: 2286 and a capsid encoded by a nucleic acid sequence comprising SEQ ID NO: 2. In some embodiments, the AAV particle comprises a viral genome comprising SEQ ID NO: 2286 and a capsid comprising SEQ ID NO: 4534. In some embodiments, the AAV particle comprises a viral genome comprising SEQ ID NO: 2286 and a capsid encoded by a nucleic acid sequence comprising SEQ ID NO: 3. In some embodiments, the AAV particle comprises a viral genome comprising SEQ ID NO: 2286 and a capsid comprising SEQ ID NO: 138. In some embodiments, the AAV particle comprises a viral genome comprising SEQ ID NO: 2286 and a capsid encoded by a nucleic acid sequence comprising SEQ ID NO: 137. In some embodiments, the AAV particle comprises a viral genome comprising SEQ ID NO: 2286 and a capsid comprising SEQ ID NO: 5. In some embodiments, the AAV particle comprises a viral genome comprising SEQ ID NO: 2286 and a capsid encoded by a nucleic acid sequence comprising SEQ ID NO: 6. In some embodiments, the AAV particle comprises a viral genome comprising SEQ ID NO: 2286 and a capsid comprising SEQ ID NO: 4. In some embodiments, the AAV particle comprises a viral genome comprising SEQ ID NO: 2286 and a capsid comprising SEQ ID NO: 11. In some embodiments, the AAV particle comprises a viral genome comprising SEQ ID NO: 2286 and a capsid comprising SEQ ID NO: 2679. In some embodiments, the AAV particle comprises a viral genome comprising SEQ ID NO: 2286 and a capsid comprising SEQ ID NO: 2809. In some embodiments, the AAV particle comprises a viral genome comprising SEQ ID NO: 2286 and a capsid comprising SEQ ID NO: 2871.

In some embodiments, the AAV particle comprises a viral genome comprising SEQ ID NO: 2287 and a VOY101 capsid. In some embodiments, the AAV particle comprises a viral genome comprising SEQ ID NO: 2287 and a VOY201 capsid. In some embodiments, the AAV particle comprises a viral genome comprising SEQ ID NO: 2287 and an AAV9 capsid. In some embodiments, the AAV particle comprises a viral genome comprising SEQ ID NO: 2287 and an AAV9K449R capsid. In some embodiments, the AAV particle comprises a viral genome comprising SEQ ID NO: 2287 and an AAVPHP.B capsid. In some embodiments, the AAV particle comprises a viral genome comprising SEQ ID NO: 2287 and an AAVPHP.N capsid. In some embodiments, the AAV particle comprises a viral genome comprising SEQ ID NO: 2287 and an AAV2 variant capsid. In some embodiments, the AAV particle comprises a viral genome comprising SEQ ID NO: 2287 and an AAV2/3 variant capsid. In some embodiments, the AAV particle comprises a viral genome comprising SEQ ID NO: 2287 and a capsid comprising SEQ ID NO: 1. In some embodiments, the AAV particle comprises a viral genome comprising SEQ ID NO: 2287 and a capsid encoded by a nucleic acid sequence comprising SEQ ID NO: 2. In some embodiments, the AAV particle comprises a viral genome comprising SEQ ID NO: 2287 and a capsid comprising SEQ ID NO: 4534. In some embodiments, the AAV particle comprises a viral genome comprising SEQ ID NO: 2287 and a capsid encoded by a nucleic acid sequence comprising SEQ ID NO: 3. In some embodiments, the AAV particle comprises a viral genome comprising SEQ ID NO: 2287 and a capsid comprising SEQ ID NO: 138. In some embodiments, the AAV particle comprises a viral genome comprising SEQ ID NO: 2287 and a capsid encoded by a nucleic acid sequence comprising SEQ ID NO: 137. In some embodiments, the AAV particle comprises a viral genome comprising SEQ ID NO: 2287 and a capsid comprising SEQ ID NO: 5. In some embodiments, the AAV particle comprises a viral genome comprising SEQ ID NO: 2287 and a capsid encoded by a nucleic acid sequence comprising SEQ ID NO: 6. In some embodiments, the AAV particle comprises a viral genome comprising SEQ ID NO: 2287 and a capsid comprising SEQ ID NO: 4. In some embodiments, the AAV particle comprises a viral genome comprising SEQ ID NO: 2287 and a capsid comprising SEQ ID NO: 11. In some embodiments, the AAV particle comprises a viral genome comprising SEQ ID NO: 2287 and a capsid comprising SEQ ID NO: 2679. In some embodiments, the AAV particle comprises a viral genome comprising SEQ ID NO: 2287 and a capsid comprising SEQ ID NO: 2809. In some embodiments, the AAV particle comprises a viral genome comprising SEQ ID NO: 2287 and a capsid comprising SEQ ID NO: 2871.

In some embodiments, the AAV particle comprises a viral genome comprising SEQ ID NO: 2288 and a VOY101 capsid. In some embodiments, the AAV particle comprises a viral genome comprising SEQ ID NO: 2288 and a VOY201 capsid. In some embodiments, the AAV particle comprises a viral genome comprising SEQ ID NO: 2288 and an AAV9 capsid. In some embodiments, the AAV particle comprises a viral genome comprising SEQ ID NO: 2288 and an AAV9K449R capsid. In some embodiments, the AAV particle comprises a viral genome comprising SEQ ID NO: 2288 and an AAVPHP.B capsid. In some embodiments, the AAV particle comprises a viral genome comprising SEQ ID NO: 2288 and an AAVPHP.N capsid. In some embodiments, the AAV particle comprises a viral genome comprising SEQ ID NO: 2288 and an AAV2 variant capsid. In some embodiments, the AAV particle comprises a viral genome comprising SEQ ID NO: 2288 and an AAV2/3 variant capsid. In some embodiments, the AAV particle comprises a viral genome comprising SEQ ID NO: 2288 and a capsid comprising SEQ ID NO: 1. In some embodiments, the AAV particle comprises a viral genome comprising SEQ ID NO: 2288 and a capsid encoded by a nucleic acid sequence comprising SEQ ID NO: 2. In some embodiments, the AAV particle comprises a viral genome comprising SEQ ID NO: 2288 and a capsid comprising SEQ ID NO: 4534. In some embodiments, the AAV particle comprises a viral genome comprising SEQ ID NO: 2288 and a capsid encoded by a nucleic acid sequence comprising SEQ ID NO: 3. In some embodiments, the AAV particle comprises a viral genome comprising SEQ ID NO: 2288 and a capsid comprising SEQ ID NO: 138. In some embodiments, the AAV particle comprises a viral genome comprising SEQ ID NO: 2288 and a capsid encoded by a nucleic acid sequence comprising SEQ ID NO: 137. In some embodiments, the AAV particle comprises a viral genome comprising SEQ ID NO: 2288 and a capsid comprising SEQ ID NO: 5. In some embodiments, the AAV particle comprises a viral genome comprising SEQ ID NO: 2288 and a capsid encoded by a nucleic acid sequence comprising SEQ ID NO: 6. In some embodiments, the AAV particle comprises a viral genome comprising SEQ ID NO: 2288 and a capsid comprising SEQ ID NO: 4. In some embodiments, the AAV particle comprises a viral genome comprising SEQ ID NO: 2288 and a capsid comprising SEQ ID NO: 11. In some embodiments, the AAV particle comprises a viral genome comprising SEQ ID NO: 2288 and a capsid comprising SEQ ID NO: 2679. In some embodiments, the AAV particle comprises a viral genome comprising SEQ ID NO: 2288 and a capsid comprising SEQ ID NO: 2809. In some embodiments, the AAV particle comprises a viral genome comprising SEQ ID NO: 2288 and a capsid comprising SEQ ID NO: 2871.

In some embodiments, the AAV particle comprises a viral genome comprising SEQ ID NO: 2289 and a VOY101 capsid. In some embodiments, the AAV particle comprises a viral genome comprising SEQ ID NO: 2289 and a VOY201 capsid. In some embodiments, the AAV particle comprises a viral genome comprising SEQ ID NO: 2289 and an AAV9 capsid. In some embodiments, the AAV particle comprises a viral genome comprising SEQ ID NO: 2289 and an AAV9K449R capsid. In some embodiments, the AAV particle comprises a viral genome comprising SEQ ID NO: 2289 and an AAVPHP.B capsid. In some embodiments, the AAV particle comprises a viral genome comprising SEQ ID NO: 2289 and an AAVPHP.N capsid. In some embodiments, the AAV particle comprises a viral genome comprising SEQ ID NO: 2289 and an AAV2 variant capsid. In some embodiments, the AAV particle comprises a viral genome comprising SEQ ID NO: 2289 and an AAV2/3 variant capsid. In some embodiments, the AAV particle comprises a viral genome comprising SEQ ID NO: 2289 and a capsid comprising SEQ ID NO: 1. In some embodiments, the AAV particle comprises a viral genome comprising SEQ ID NO: 2289 and a capsid encoded by a nucleic acid sequence comprising SEQ ID NO: 2. In some embodiments, the AAV particle comprises a viral genome comprising SEQ ID NO: 2289 and a capsid comprising SEQ ID NO: 4534. In some embodiments, the AAV particle comprises a viral genome comprising SEQ ID NO: 2289 and a capsid encoded by a nucleic acid sequence comprising SEQ ID NO: 3. In some embodiments, the AAV particle comprises a viral genome comprising SEQ ID NO: 2289 and a capsid comprising SEQ ID NO: 138. In some embodiments, the AAV particle comprises a viral genome comprising SEQ ID NO: 2289 and a capsid encoded by a nucleic acid sequence comprising SEQ ID NO: 137. In some embodiments, the AAV particle comprises a viral genome comprising SEQ ID NO: 2289 and a capsid comprising SEQ ID NO: 5. In some embodiments, the AAV particle comprises a viral genome comprising SEQ ID NO: 2289 and a capsid encoded by a nucleic acid sequence comprising SEQ ID NO: 6. In some embodiments, the AAV particle comprises a viral genome comprising SEQ ID NO: 2289 and a capsid comprising SEQ ID NO: 4. In some embodiments, the AAV particle comprises a viral genome comprising SEQ ID NO: 2289 and a capsid comprising SEQ ID NO: 11. In some embodiments, the AAV particle comprises a viral genome comprising SEQ ID NO: 2289 and a capsid comprising SEQ ID NO: 2679. In some embodiments, the AAV particle comprises a viral genome comprising SEQ ID NO: 2289 and a capsid comprising SEQ ID NO: 2809. In some embodiments, the AAV particle comprises a viral genome comprising SEQ ID NO: 2289 and a capsid comprising SEQ ID NO: 2871.

In some embodiments, the AAV particle comprises a viral genome comprising SEQ ID NO: 2290 and a VOY101 capsid. In some embodiments, the AAV particle comprises a viral genome comprising SEQ ID NO: 2290 and a VOY201 capsid. In some embodiments, the AAV particle comprises a viral genome comprising SEQ ID NO: 2290 and an AAV9 capsid. In some embodiments, the AAV particle comprises a viral genome comprising SEQ ID NO: 2290 and an AAV9K449R capsid. In some embodiments, the AAV particle comprises a viral genome comprising SEQ ID NO: 2290 and an AAVPHP.B capsid. In some embodiments, the AAV particle comprises a viral genome comprising SEQ ID NO: 2290 and an AAVPHP.N capsid. In some embodiments, the AAV particle comprises a viral genome comprising SEQ ID NO: 2290 and an AAV2 variant capsid. In some embodiments, the AAV particle comprises a viral genome comprising SEQ ID NO: 2290 and an AAV2/3 variant capsid. In some embodiments, the AAV particle comprises a viral genome comprising SEQ ID NO: 2290 and a capsid comprising SEQ ID NO: 1. In some embodiments, the AAV particle comprises a viral genome comprising SEQ ID NO: 2290 and a capsid encoded by a nucleic acid sequence comprising SEQ ID NO: 2. In some embodiments, the AAV particle comprises a viral genome comprising SEQ ID NO: 2290 and a capsid comprising SEQ ID NO: 4534. In some embodiments, the AAV particle comprises a viral genome comprising SEQ ID NO: 2290 and a capsid encoded by a nucleic acid sequence comprising SEQ ID NO: 3. In some embodiments, the AAV particle comprises a viral genome comprising SEQ ID NO: 2290 and a capsid comprising SEQ ID NO: 138. In some embodiments, the AAV particle comprises a viral genome comprising SEQ ID NO: 2290 and a capsid encoded by a nucleic acid sequence comprising SEQ ID NO: 137. In some embodiments, the AAV particle comprises a viral genome comprising SEQ ID NO: 2290 and a capsid comprising SEQ ID NO: 5. In some embodiments, the AAV particle comprises a viral genome comprising SEQ ID NO: 2290 and a capsid encoded by a nucleic acid sequence comprising SEQ ID NO: 6. In some embodiments, the AAV particle comprises a viral genome comprising SEQ ID NO: 2290 and a capsid comprising SEQ ID NO: 4. In some embodiments, the AAV particle comprises a viral genome comprising SEQ ID NO: 2290 and a capsid comprising SEQ ID NO: 11. In some embodiments, the AAV particle comprises a viral genome comprising SEQ ID NO: 2290 and a capsid comprising SEQ ID NO: 2679. In some embodiments, the AAV particle comprises a viral genome comprising SEQ ID NO: 2290 and a capsid comprising SEQ ID NO: 2809. In some embodiments, the AAV particle comprises a viral genome comprising SEQ ID NO: 2290 and a capsid comprising SEQ ID NO: 2871.

In some embodiments, the AAV particle comprises a viral genome comprising SEQ ID NO: 2291 and a VOY101 capsid. In some embodiments, the AAV particle comprises a viral genome comprising SEQ ID NO: 2291 and a VOY201 capsid. In some embodiments, the AAV particle comprises a viral genome comprising SEQ ID NO: 2291 and an AAV9 capsid. In some embodiments, the AAV particle comprises a viral genome comprising SEQ ID NO: 2291 and an AAV9K449R capsid. In some embodiments, the AAV particle comprises a viral genome comprising SEQ ID NO: 2291 and an AAVPHP.B capsid. In some embodiments, the AAV particle comprises a viral genome comprising SEQ ID NO: 2291 and an AAVPHP.N capsid. In some embodiments, the AAV particle comprises a viral genome comprising SEQ ID NO: 2291 and an AAV2 variant capsid. In some embodiments, the AAV particle comprises a viral genome comprising SEQ ID NO: 2291 and an AAV2/3 variant capsid. In some embodiments, the AAV particle comprises a viral genome comprising SEQ ID NO: 2291 and a capsid comprising SEQ ID NO: 1. In some embodiments, the AAV particle comprises a viral genome comprising SEQ ID NO: 2291 and a capsid encoded by a nucleic acid sequence comprising SEQ ID NO: 2. In some embodiments, the AAV particle comprises a viral genome comprising SEQ ID NO: 2291 and a capsid comprising SEQ ID NO: 4534. In some embodiments, the AAV particle comprises a viral genome comprising SEQ ID NO: 2291 and a capsid encoded by a nucleic acid sequence comprising SEQ ID NO: 3. In some embodiments, the AAV particle comprises a viral genome comprising SEQ ID NO: 2291 and a capsid comprising SEQ ID NO: 138. In some embodiments, the AAV particle comprises a viral genome comprising SEQ ID NO: 2291 and a capsid encoded by a nucleic acid sequence comprising SEQ ID NO: 137. In some embodiments, the AAV particle comprises a viral genome comprising SEQ ID NO: 2291 and a capsid comprising SEQ ID NO: 5. In some embodiments, the AAV particle comprises a viral genome comprising SEQ ID NO: 2291 and a capsid encoded by a nucleic acid sequence comprising SEQ ID NO: 6. In some embodiments, the AAV particle comprises a viral genome comprising SEQ ID NO: 2291 and a capsid comprising SEQ ID NO: 4. In some embodiments, the AAV particle comprises a viral genome comprising SEQ ID NO: 2291 and a capsid comprising SEQ ID NO: 11. In some embodiments, the AAV particle comprises a viral genome comprising SEQ ID NO: 2291 and a capsid comprising SEQ ID NO: 2679. In some embodiments, the AAV particle comprises a viral genome comprising SEQ ID NO: 2291 and a capsid comprising SEQ ID NO: 2809. In some embodiments, the AAV particle comprises a viral genome comprising SEQ ID NO: 2291 and a capsid comprising SEQ ID NO: 2871.

In some embodiments, the AAV particle comprises a viral genome comprising SEQ ID NO: 2292 and a VOY101 capsid. In some embodiments, the AAV particle comprises a viral genome comprising SEQ ID NO: 2292 and a VOY201 capsid. In some embodiments, the AAV particle comprises a viral genome comprising SEQ ID NO: 2292 and an AAV9 capsid. In some embodiments, the AAV particle comprises a viral genome comprising SEQ ID NO: 2292 and an AAV9K449R capsid. In some embodiments, the AAV particle comprises a viral genome comprising SEQ ID NO: 2292 and an AAVPHP.B capsid. In some embodiments, the AAV particle comprises a viral genome comprising SEQ ID NO: 2292 and an AAVPHP.N capsid. In some embodiments, the AAV particle comprises a viral genome comprising SEQ ID NO: 2292 and an AAV2 variant capsid. In some embodiments, the AAV particle comprises a viral genome comprising SEQ ID NO: 2292 and an AAV2/3 variant capsid. In some embodiments, the AAV particle comprises a viral genome comprising SEQ ID NO: 2292 and a capsid comprising SEQ ID NO: 1. In some embodiments, the AAV particle comprises a viral genome comprising SEQ ID NO: 2292 and a capsid encoded by a nucleic acid sequence comprising SEQ ID NO: 2. In some embodiments, the AAV particle comprises a viral genome comprising SEQ ID NO: 2292 and a capsid comprising SEQ ID NO: 4534. In some embodiments, the AAV particle comprises a viral genome comprising SEQ ID NO: 2292 and a capsid encoded by a nucleic acid sequence comprising SEQ ID NO: 3. In some embodiments, the AAV particle comprises a viral genome comprising SEQ ID NO: 2292 and a capsid comprising SEQ ID NO: 138. In some embodiments, the AAV particle comprises a viral genome comprising SEQ ID NO: 2292 and a capsid encoded by a nucleic acid sequence comprising SEQ ID NO: 137. In some embodiments, the AAV particle comprises a viral genome comprising SEQ ID NO: 2292 and a capsid comprising SEQ ID NO: 5. In some embodiments, the AAV particle comprises a viral genome comprising SEQ ID NO: 2292 and a capsid encoded by a nucleic acid sequence comprising SEQ ID NO: 6. In some embodiments, the AAV particle comprises a viral genome comprising SEQ ID NO: 2292 and a capsid comprising SEQ ID NO: 4. In some embodiments, the AAV particle comprises a viral genome comprising SEQ ID NO: 2292 and a capsid comprising SEQ ID NO: 11. In some embodiments, the AAV particle comprises a viral genome comprising SEQ ID NO: 2292 and a capsid comprising SEQ ID NO: 2679. In some embodiments, the AAV particle comprises a viral genome comprising SEQ ID NO: 2292 and a capsid comprising SEQ ID NO: 2809. In some embodiments, the AAV particle comprises a viral genome comprising SEQ ID NO: 2292 and a capsid comprising SEQ ID NO: 2871.

In some embodiments, the AAV particle comprises a viral genome comprising SEQ ID NO: 2293 and a VOY101 capsid. In some embodiments, the AAV particle comprises a viral genome comprising SEQ ID NO: 2293 and a VOY201 capsid. In some embodiments, the AAV particle comprises a viral genome comprising SEQ ID NO: 2293 and an AAV9 capsid. In some embodiments, the AAV particle comprises a viral genome comprising SEQ ID NO: 2293 and an AAV9K449R capsid. In some embodiments, the AAV particle comprises a viral genome comprising SEQ ID NO: 2293 and an AAVPHP.B capsid. In some embodiments, the AAV particle comprises a viral genome comprising SEQ ID NO: 2293 and an AAVPHP.N capsid. In some embodiments, the AAV particle comprises a viral genome comprising SEQ ID NO: 2293 and an AAV2 variant capsid. In some embodiments, the AAV particle comprises a viral genome comprising SEQ ID NO: 2293 and an AAV2/3 variant capsid. In some embodiments, the AAV particle comprises a viral genome comprising SEQ ID NO: 2293 and a capsid comprising SEQ ID NO: 1. In some embodiments, the AAV particle comprises a viral genome comprising SEQ ID NO: 2293 and a capsid encoded by a nucleic acid sequence comprising SEQ ID NO: 2. In some embodiments, the AAV particle comprises a viral genome comprising SEQ ID NO: 2293 and a capsid comprising SEQ ID NO: 4534. In some embodiments, the AAV particle comprises a viral genome comprising SEQ ID NO: 2293 and a capsid encoded by a nucleic acid sequence comprising SEQ ID NO: 3. In some embodiments, the AAV particle comprises a viral genome comprising SEQ ID NO: 2293 and a capsid comprising SEQ ID NO: 138. In some embodiments, the AAV particle comprises a viral genome comprising SEQ ID NO: 2293 and a capsid encoded by a nucleic acid sequence comprising SEQ ID NO: 137. In some embodiments, the AAV particle comprises a viral genome comprising SEQ ID NO: 2293 and a capsid comprising SEQ ID NO: 5. In some embodiments, the AAV particle comprises a viral genome comprising SEQ ID NO: 2293 and a capsid encoded by a nucleic acid sequence comprising SEQ ID NO: 6. In some embodiments, the AAV particle comprises a viral genome comprising SEQ ID NO: 2293 and a capsid comprising SEQ ID NO: 4. In some embodiments, the AAV particle comprises a viral genome comprising SEQ ID NO: 2293 and a capsid comprising SEQ ID NO: 11. In some embodiments, the AAV particle comprises a viral genome comprising SEQ ID NO: 2293 and a capsid comprising SEQ ID NO: 2679. In some embodiments, the AAV particle comprises a viral genome comprising SEQ ID NO: 2293 and a capsid comprising SEQ ID NO: 2809. In some embodiments, the AAV particle comprises a viral genome comprising SEQ ID NO: 2293 and a capsid comprising SEQ ID NO: 2871.

In some embodiments, the AAV particle comprises a viral genome comprising SEQ ID NO: 2294 and a VOY101 capsid. In some embodiments, the AAV particle comprises a viral genome comprising SEQ ID NO: 2294 and a VOY201 capsid. In some embodiments, the AAV particle comprises a viral genome comprising SEQ ID NO: 2294 and an AAV9 capsid. In some embodiments, the AAV particle comprises a viral genome comprising SEQ ID NO: 2294 and an AAV9K449R capsid. In some embodiments, the AAV particle comprises a viral genome comprising SEQ ID NO: 2294 and an AAVPHP.B capsid. In some embodiments, the AAV particle comprises a viral genome comprising SEQ ID NO: 2294 and an AAVPHP.N capsid. In some embodiments, the AAV particle comprises a viral genome comprising SEQ ID NO: 2294 and an AAV2 variant capsid. In some embodiments, the AAV particle comprises a viral genome comprising SEQ ID NO: 2294 and an AAV2/3 variant capsid. In some embodiments, the AAV particle comprises a viral genome comprising SEQ ID NO: 2294 and a capsid comprising SEQ ID NO: 1. In some embodiments, the AAV particle comprises a viral genome comprising SEQ ID NO: 2294 and a capsid encoded by a nucleic acid sequence comprising SEQ ID NO: 2. In some embodiments, the AAV particle comprises a viral genome comprising SEQ ID NO: 2294 and a capsid comprising SEQ ID NO: 4534. In some embodiments, the AAV particle comprises a viral genome comprising SEQ ID NO: 2294 and a capsid encoded by a nucleic acid sequence comprising SEQ ID NO: 3. In some embodiments, the AAV particle comprises a viral genome comprising SEQ ID NO: 2294 and a capsid comprising SEQ ID NO: 138. In some embodiments, the AAV particle comprises a viral genome comprising SEQ ID NO: 2294 and a capsid encoded by a nucleic acid sequence comprising SEQ ID NO: 137. In some embodiments, the AAV particle comprises a viral genome comprising SEQ ID NO: 2294 and a capsid comprising SEQ ID NO: 5. In some embodiments, the AAV particle comprises a viral genome comprising SEQ ID NO: 2294 and a capsid encoded by a nucleic acid sequence comprising SEQ ID NO: 6. In some embodiments, the AAV particle comprises a viral genome comprising SEQ ID NO: 2294 and a capsid comprising SEQ ID NO: 4. In some embodiments, the AAV particle comprises a viral genome comprising SEQ ID NO: 2294 and a capsid comprising SEQ ID NO: 11. In some embodiments, the AAV particle comprises a viral genome comprising SEQ ID NO: 2294 and a capsid comprising SEQ ID NO: 2679. In some embodiments, the AAV particle comprises a viral genome comprising SEQ ID NO: 2294 and a capsid comprising SEQ ID NO: 2809. In some embodiments, the AAV particle comprises a viral genome comprising SEQ ID NO: 2294 and a capsid comprising SEQ ID NO: 2871.

In some embodiments, the AAV particle comprises a viral genome comprising SEQ ID NO: 2295 and a VOY101 capsid. In some embodiments, the AAV particle comprises a viral genome comprising SEQ ID NO: 2295 and a VOY201 capsid. In some embodiments, the AAV particle comprises a viral genome comprising SEQ ID NO: 2295 and an AAV9 capsid. In some embodiments, the AAV particle comprises a viral genome comprising SEQ ID NO: 2295 and an AAV9K449R capsid. In some embodiments, the AAV particle comprises a viral genome comprising SEQ ID NO: 2295 and an AAVPHP.B capsid. In some embodiments, the AAV particle comprises a viral genome comprising SEQ ID NO: 2295 and an AAVPHP.N capsid. In some embodiments, the AAV particle comprises a viral genome comprising SEQ ID NO: 2295 and an AAV2 variant capsid. In some embodiments, the AAV particle comprises a viral genome comprising SEQ ID NO: 2295 and an AAV2/3 variant capsid. In some embodiments, the AAV particle comprises a viral genome comprising SEQ ID NO: 2295 and a capsid comprising SEQ ID NO: 1. In some embodiments, the AAV particle comprises a viral genome comprising SEQ ID NO: 2295 and a capsid encoded by a nucleic acid sequence comprising SEQ ID NO: 2. In some embodiments, the AAV particle comprises a viral genome comprising SEQ ID NO: 2295 and a capsid comprising SEQ ID NO: 4534. In some embodiments, the AAV particle comprises a viral genome comprising SEQ ID NO: 2295 and a capsid encoded by a nucleic acid sequence comprising SEQ ID NO: 3. In some embodiments, the AAV particle comprises a viral genome comprising SEQ ID NO: 2295 and a capsid comprising SEQ ID NO: 138. In some embodiments, the AAV particle comprises a viral genome comprising SEQ ID NO: 2295 and a capsid encoded by a nucleic acid sequence comprising SEQ ID NO: 137. In some embodiments, the AAV particle comprises a viral genome comprising SEQ ID NO: 2295 and a capsid comprising SEQ ID NO: 5. In some embodiments, the AAV particle comprises a viral genome comprising SEQ ID NO: 2295 and a capsid encoded by a nucleic acid sequence comprising SEQ ID NO: 6. In some embodiments, the AAV particle comprises a viral genome comprising SEQ ID NO: 2295 and a capsid comprising SEQ ID NO: 4. In some embodiments, the AAV particle comprises a viral genome comprising SEQ ID NO: 2295 and a capsid comprising SEQ ID NO: 11. In some embodiments, the AAV particle comprises a viral genome comprising SEQ ID NO: 2295 and a capsid comprising SEQ ID NO: 2679. In some embodiments, the AAV particle comprises a viral genome comprising SEQ ID NO: 2295 and a capsid comprising SEQ ID NO: 2809. In some embodiments, the AAV particle comprises a viral genome comprising SEQ ID NO: 2295 and a capsid comprising SEQ ID NO: 2871.

In some embodiments, the AAV particle comprises a viral genome comprising SEQ ID NO: 2296 and a VOY101 capsid. In some embodiments, the AAV particle comprises a viral genome comprising SEQ ID NO: 2296 and a VOY201 capsid. In some embodiments, the AAV particle comprises a viral genome comprising SEQ ID NO: 2296 and an AAV9 capsid. In some embodiments, the AAV particle comprises a viral genome comprising SEQ ID NO: 2296 and an AAV9K449R capsid. In some embodiments, the AAV particle comprises a viral genome comprising SEQ ID NO: 2296 and an AAVPHP.B capsid. In some embodiments, the AAV particle comprises a viral genome comprising SEQ ID NO: 2296 and an AAVPHP.N capsid. In some embodiments, the AAV particle comprises a viral genome comprising SEQ ID NO: 2296 and an AAV2 variant capsid. In some embodiments, the AAV particle comprises a viral genome comprising SEQ ID NO: 2296 and an AAV2/3 variant capsid. In some embodiments, the AAV particle comprises a viral genome comprising SEQ ID NO: 2296 and a capsid comprising SEQ ID NO: 1. In some embodiments, the AAV particle comprises a viral genome comprising SEQ ID NO: 2296 and a capsid encoded by a nucleic acid sequence comprising SEQ ID NO: 2. In some embodiments, the AAV particle comprises a viral genome comprising SEQ ID NO: 2296 and a capsid comprising SEQ ID NO: 4534. In some embodiments, the AAV particle comprises a viral genome comprising SEQ ID NO: 2296 and a capsid encoded by a nucleic acid sequence comprising SEQ ID NO: 3. In some embodiments, the AAV particle comprises a viral genome comprising SEQ ID NO: 2296 and a capsid comprising SEQ ID NO: 138. In some embodiments, the AAV particle comprises a viral genome comprising SEQ ID NO: 2296 and a capsid encoded by a nucleic acid sequence comprising SEQ ID NO: 137. In some embodiments, the AAV particle comprises a viral genome comprising SEQ ID NO: 2296 and a capsid comprising SEQ ID NO: 5. In some embodiments, the AAV particle comprises a viral genome comprising SEQ ID NO: 2296 and a capsid encoded by a nucleic acid sequence comprising SEQ ID NO: 6. In some embodiments, the AAV particle comprises a viral genome comprising SEQ ID NO: 2296 and a capsid comprising SEQ ID NO: 4. In some embodiments, the AAV particle comprises a viral genome comprising SEQ ID NO: 2296 and a capsid comprising SEQ ID NO: 11. In some embodiments, the AAV particle comprises a viral genome comprising SEQ ID NO: 2296 and a capsid comprising SEQ ID NO: 2679. In some embodiments, the AAV particle comprises a viral genome comprising SEQ ID NO: 2296 and a capsid comprising SEQ ID NO: 2809. In some embodiments, the AAV particle comprises a viral genome comprising SEQ ID NO: 2296 and a capsid comprising SEQ ID NO: 2871.

In some embodiments, the AAV particle comprises a viral genome comprising SEQ ID NO: 2297 and a VOY101 capsid. In some embodiments, the AAV particle comprises a viral genome comprising SEQ ID NO: 2297 and a VOY201 capsid. In some embodiments, the AAV particle comprises a viral genome comprising SEQ ID NO: 2297 and an AAV9 capsid. In some embodiments, the AAV particle comprises a viral genome comprising SEQ ID NO: 2297 and an AAV9K449R capsid. In some embodiments, the AAV particle comprises a viral genome comprising SEQ ID NO: 2297 and an AAVPHP.B capsid. In some embodiments, the AAV particle comprises a viral genome comprising SEQ ID NO: 2297 and an AAVPHP.N capsid. In some embodiments, the AAV particle comprises a viral genome comprising SEQ ID NO: 2297 and an AAV2 variant capsid. In some embodiments, the AAV particle comprises a viral genome comprising SEQ ID NO: 2297 and an AAV2/3 variant capsid. In some embodiments, the AAV particle comprises a viral genome comprising SEQ ID NO: 2297 and a capsid comprising SEQ ID NO: 1. In some embodiments, the AAV particle comprises a viral genome comprising SEQ ID NO: 2297 and a capsid encoded by a nucleic acid sequence comprising SEQ ID NO: 2. In some embodiments, the AAV particle comprises a viral genome comprising SEQ ID NO: 2297 and a capsid comprising SEQ ID NO: 4534. In some embodiments, the AAV particle comprises a viral genome comprising SEQ ID NO: 2297 and a capsid encoded by a nucleic acid sequence comprising SEQ ID NO: 3. In some embodiments, the AAV particle comprises a viral genome comprising SEQ ID NO: 2297 and a capsid comprising SEQ ID NO: 138. In some embodiments, the AAV particle comprises a viral genome comprising SEQ ID NO: 2297 and a capsid encoded by a nucleic acid sequence comprising SEQ ID NO: 137. In some embodiments, the AAV particle comprises a viral genome comprising SEQ ID NO: 2297 and a capsid comprising SEQ ID NO: 5. In some embodiments, the AAV particle comprises a viral genome comprising SEQ ID NO: 2297 and a capsid encoded by a nucleic acid sequence comprising SEQ ID NO: 6. In some embodiments, the AAV particle comprises a viral genome comprising SEQ ID NO: 2297 and a capsid comprising SEQ ID NO: 4. In some embodiments, the AAV particle comprises a viral genome comprising SEQ ID NO: 2297 and a capsid comprising SEQ ID NO: 11. In some embodiments, the AAV particle comprises a viral genome comprising SEQ ID NO: 2297 and a capsid comprising SEQ ID NO: 2679. In some embodiments, the AAV particle comprises a viral genome comprising SEQ ID NO: 2297 and a capsid comprising SEQ ID NO: 2809. In some embodiments, the AAV particle comprises a viral genome comprising SEQ ID NO: 2297 and a capsid comprising SEQ ID NO: 2871.

In some embodiments, the AAV particle comprises a viral genome comprising SEQ ID NO: 2298 and a VOY101 capsid. In some embodiments, the AAV particle comprises a viral genome comprising SEQ ID NO: 2298 and a VOY201 capsid. In some embodiments, the AAV particle comprises a viral genome comprising SEQ ID NO: 2298 and an AAV9 capsid. In some embodiments, the AAV particle comprises a viral genome comprising SEQ ID NO: 2298 and an AAV9K449R capsid. In some embodiments, the AAV particle comprises a viral genome comprising SEQ ID NO: 2298 and an AAVPHP.B capsid. In some embodiments, the AAV particle comprises a viral genome comprising SEQ ID NO: 2298 and an AAVPHP.N capsid. In some embodiments, the AAV particle comprises a viral genome comprising SEQ ID NO: 2298 and an AAV2 variant capsid. In some embodiments, the AAV particle comprises a viral genome comprising SEQ ID NO: 2298 and an AAV2/3 variant capsid. In some embodiments, the AAV particle comprises a viral genome comprising SEQ ID NO: 2298 and a capsid comprising SEQ ID NO: 1. In some embodiments, the AAV particle comprises a viral genome comprising SEQ ID NO: 2298 and a capsid encoded by a nucleic acid sequence comprising SEQ ID NO: 2. In some embodiments, the AAV particle comprises a viral genome comprising SEQ ID NO: 2298 and a capsid comprising SEQ ID NO: 4534. In some embodiments, the AAV particle comprises a viral genome comprising SEQ ID NO: 2298 and a capsid encoded by a nucleic acid sequence comprising SEQ ID NO: 3. In some embodiments, the AAV particle comprises a viral genome comprising SEQ ID NO: 2298 and a capsid comprising SEQ ID NO: 138. In some embodiments, the AAV particle comprises a viral genome comprising SEQ ID NO: 2298 and a capsid encoded by a nucleic acid sequence comprising SEQ ID NO: 137. In some embodiments, the AAV particle comprises a viral genome comprising SEQ ID NO: 2298 and a capsid comprising SEQ ID NO: 5. In some embodiments, the AAV particle comprises a viral genome comprising SEQ ID NO: 2298 and a capsid encoded by a nucleic acid sequence comprising SEQ ID NO: 6. In some embodiments, the AAV particle comprises a viral genome comprising SEQ ID NO: 2298 and a capsid comprising SEQ ID NO: 4. In some embodiments, the AAV particle comprises a viral genome comprising SEQ ID NO: 2298 and a capsid comprising SEQ ID NO: 11. In some embodiments, the AAV particle comprises a viral genome comprising SEQ ID NO: 2298 and a capsid comprising SEQ ID NO: 2679. In some embodiments, the AAV particle comprises a viral genome comprising SEQ ID NO: 2298 and a capsid comprising SEQ ID NO: 2809. In some embodiments, the AAV particle comprises a viral genome comprising SEQ ID NO: 2298 and a capsid comprising SEQ ID NO: 2871.

In some embodiments, the AAV particle comprises a viral genome comprising SEQ ID NO: 2299 and a VOY101 capsid. In some embodiments, the AAV particle comprises a viral genome comprising SEQ ID NO: 2299 and a VOY201 capsid. In some embodiments, the AAV particle comprises a viral genome comprising SEQ ID NO: 2299 and an AAV9 capsid. In some embodiments, the AAV particle comprises a viral genome comprising SEQ ID NO: 2299 and an AAV9K449R capsid. In some embodiments, the AAV particle comprises a viral genome comprising SEQ ID NO: 2299 and an AAVPHP.B capsid. In some embodiments, the AAV particle comprises a viral genome comprising SEQ ID NO: 2299 and an AAVPHP.N capsid. In some embodiments, the AAV particle comprises a viral genome comprising SEQ ID NO: 2299 and an AAV2 variant capsid. In some embodiments, the AAV particle comprises a viral genome comprising SEQ ID NO: 2299 and an AAV2/3 variant capsid. In some embodiments, the AAV particle comprises a viral genome comprising SEQ ID NO: 2299 and a capsid comprising SEQ ID NO: 1. In some embodiments, the AAV particle comprises a viral genome comprising SEQ ID NO: 2299 and a capsid encoded by a nucleic acid sequence comprising SEQ ID NO: 2. In some embodiments, the AAV particle comprises a viral genome comprising SEQ ID NO: 2299 and a capsid comprising SEQ ID NO: 4534. In some embodiments, the AAV particle comprises a viral genome comprising SEQ ID NO: 2299 and a capsid encoded by a nucleic acid sequence comprising SEQ ID NO: 3. In some embodiments, the AAV particle comprises a viral genome comprising SEQ ID NO: 2299 and a capsid comprising SEQ ID NO: 138. In some embodiments, the AAV particle comprises a viral genome comprising SEQ ID NO: 2299 and a capsid encoded by a nucleic acid sequence comprising SEQ ID NO: 137. In some embodiments, the AAV particle comprises a viral genome comprising SEQ ID NO: 2299 and a capsid comprising SEQ ID NO: 5. In some embodiments, the AAV particle comprises a viral genome comprising SEQ ID NO: 2299 and a capsid encoded by a nucleic acid sequence comprising SEQ ID NO: 6. In some embodiments, the AAV particle comprises a viral genome comprising SEQ ID NO: 2299 and a capsid comprising SEQ ID NO: 4. In some embodiments, the AAV particle comprises a viral genome comprising SEQ ID NO: 2299 and a capsid comprising SEQ ID NO: 11. In some embodiments, the AAV particle comprises a viral genome comprising SEQ ID NO: 2299 and a capsid comprising SEQ ID NO: 2679. In some embodiments, the AAV particle comprises a viral genome comprising SEQ ID NO: 2299 and a capsid comprising SEQ ID NO: 2809. In some embodiments, the AAV particle comprises a viral genome comprising SEQ ID NO: 2299 and a capsid comprising SEQ ID NO: 2871.

In some embodiments, the AAV particle comprises a viral genome comprising SEQ ID NO: 2300 and a VOY101 capsid. In some embodiments, the AAV particle comprises a viral genome comprising SEQ ID NO: 2300 and a VOY201 capsid. In some embodiments, the AAV particle comprises a viral genome comprising SEQ ID NO: 2300 and an AAV9 capsid. In some embodiments, the AAV particle comprises a viral genome comprising SEQ ID NO: 2300 and an AAV9K449R capsid. In some embodiments, the AAV particle comprises a viral genome comprising SEQ ID NO: 2300 and an AAVPHP.B capsid. In some embodiments, the AAV particle comprises a viral genome comprising SEQ ID NO: 2300 and an AAVPHP.N capsid. In some embodiments, the AAV particle comprises a viral genome comprising SEQ ID NO: 2300 and an AAV2 variant capsid. In some embodiments, the AAV particle comprises a viral genome comprising SEQ ID NO: 2300 and an AAV2/3 variant capsid. In some embodiments, the AAV particle comprises a viral genome comprising SEQ ID NO: 2300 and a capsid comprising SEQ ID NO: 1. In some embodiments, the AAV particle comprises a viral genome comprising SEQ ID NO: 2300 and a capsid encoded by a nucleic acid sequence comprising SEQ ID NO: 2. In some embodiments, the AAV particle comprises a viral genome comprising SEQ ID NO: 2300 and a capsid comprising SEQ ID NO: 4534. In some embodiments, the AAV particle comprises a viral genome comprising SEQ ID NO: 2300 and a capsid encoded by a nucleic acid sequence comprising SEQ ID NO: 3. In some embodiments, the AAV particle comprises a viral genome comprising SEQ ID NO: 2300 and a capsid comprising SEQ ID NO: 138. In some embodiments, the AAV particle comprises a viral genome comprising SEQ ID NO: 2300 and a capsid encoded by a nucleic acid sequence comprising SEQ ID NO: 137. In some embodiments, the AAV particle comprises a viral genome comprising SEQ ID NO: 2300 and a capsid comprising SEQ ID NO: 5. In some embodiments, the AAV particle comprises a viral genome comprising SEQ ID NO: 2300 and a capsid encoded by a nucleic acid sequence comprising SEQ ID NO: 6. In some embodiments, the AAV particle comprises a viral genome comprising SEQ ID NO: 2300 and a capsid comprising SEQ ID NO: 4. In some embodiments, the AAV particle comprises a viral genome comprising SEQ ID NO: 2300 and a capsid comprising SEQ ID NO: 11. In some embodiments, the AAV particle comprises a viral genome comprising SEQ ID NO: 2300 and a capsid comprising SEQ ID NO: 2679. In some embodiments, the AAV particle comprises a viral genome comprising SEQ ID NO: 2300 and a capsid comprising SEQ ID NO: 2809. In some embodiments, the AAV particle comprises a viral genome comprising SEQ ID NO: 2300 and a capsid comprising SEQ ID NO: 2871.

In some embodiments, the AAV particle comprises a viral genome comprising SEQ ID NO: 2301 and a VOY101 capsid. In some embodiments, the AAV particle comprises a viral genome comprising SEQ ID NO: 2301 and a VOY201 capsid. In some embodiments, the AAV particle comprises a viral genome comprising SEQ ID NO: 2301 and an AAV9 capsid. In some embodiments, the AAV particle comprises a viral genome comprising SEQ ID NO: 2301 and an AAV9K449R capsid. In some embodiments, the AAV particle comprises a viral genome comprising SEQ ID NO: 2301 and an AAVPHP.B capsid. In some embodiments, the AAV particle comprises a viral genome comprising SEQ ID NO: 2301 and an AAVPHP.N capsid. In some embodiments, the AAV particle comprises a viral genome comprising SEQ ID NO: 2301 and an AAV2 variant capsid. In some embodiments, the AAV particle comprises a viral genome comprising SEQ ID NO: 2301 and an AAV2/3 variant capsid. In some embodiments, the AAV particle comprises a viral genome comprising SEQ ID NO: 2301 and a capsid comprising SEQ ID NO: 1. In some embodiments, the AAV particle comprises a viral genome comprising SEQ ID NO: 2301 and a capsid encoded by a nucleic acid sequence comprising SEQ ID NO: 2. In some embodiments, the AAV particle comprises a viral genome comprising SEQ ID NO: 2301 and a capsid comprising SEQ ID NO: 4534. In some embodiments, the AAV particle comprises a viral genome comprising SEQ ID NO: 2301 and a capsid encoded by a nucleic acid sequence comprising SEQ ID NO: 3. In some embodiments, the AAV particle comprises a viral genome comprising SEQ ID NO: 2301 and a capsid comprising SEQ ID NO: 138. In some embodiments, the AAV particle comprises a viral genome comprising SEQ ID NO: 2301 and a capsid encoded by a nucleic acid sequence comprising SEQ ID NO: 137. In some embodiments, the AAV particle comprises a viral genome comprising SEQ ID NO: 2301 and a capsid comprising SEQ ID NO: 5. In some embodiments, the AAV particle comprises a viral genome comprising SEQ ID NO: 2301 and a capsid encoded by a nucleic acid sequence comprising SEQ ID NO: 6. In some embodiments, the AAV particle comprises a viral genome comprising SEQ ID NO: 2301 and a capsid comprising SEQ ID NO: 4. In some embodiments, the AAV particle comprises a viral genome comprising SEQ ID NO: 2301 and a capsid comprising SEQ ID NO: 11. In some embodiments, the AAV particle comprises a viral genome comprising SEQ ID NO: 2301 and a capsid comprising SEQ ID NO: 2679. In some embodiments, the AAV particle comprises a viral genome comprising SEQ ID NO: 2301 and a capsid comprising SEQ ID NO: 2809. In some embodiments, the AAV particle comprises a viral genome comprising SEQ ID NO: 2301 and a capsid comprising SEQ ID NO: 2871.

In some embodiments, the AAV particle comprises a viral genome comprising SEQ ID NO: 2302 and a VOY101 capsid. In some embodiments, the AAV particle comprises a viral genome comprising SEQ ID NO: 2302 and a VOY201 capsid. In some embodiments, the AAV particle comprises a viral genome comprising SEQ ID NO: 2302 and an AAV9 capsid. In some embodiments, the AAV particle comprises a viral genome comprising SEQ ID NO: 2302 and an AAV9K449R capsid. In some embodiments, the AAV particle comprises a viral genome comprising SEQ ID NO: 2302 and an AAVPHP.B capsid. In some embodiments, the AAV particle comprises a viral genome comprising SEQ ID NO: 2302 and an AAVPHP.N capsid. In some embodiments, the AAV particle comprises a viral genome comprising SEQ ID NO: 2302 and an AAV2 variant capsid. In some embodiments, the AAV particle comprises a viral genome comprising SEQ ID NO: 2302 and an AAV2/3 variant capsid. In some embodiments, the AAV particle comprises a viral genome comprising SEQ ID NO: 2302 and a capsid comprising SEQ ID NO: 1. In some embodiments, the AAV particle comprises a viral genome comprising SEQ ID NO: 2302 and a capsid encoded by a nucleic acid sequence comprising SEQ ID NO: 2. In some embodiments, the AAV particle comprises a viral genome comprising SEQ ID NO: 2302 and a capsid comprising SEQ ID NO: 4534. In some embodiments, the AAV particle comprises a viral genome comprising SEQ ID NO: 2302 and a capsid encoded by a nucleic acid sequence comprising SEQ ID NO: 3. In some embodiments, the AAV particle comprises a viral genome comprising SEQ ID NO: 2302 and a capsid comprising SEQ ID NO: 138. In some embodiments, the AAV particle comprises a viral genome comprising SEQ ID NO: 2302 and a capsid encoded by a nucleic acid sequence comprising SEQ ID NO: 137. In some embodiments, the AAV particle comprises a viral genome comprising SEQ ID NO: 2302 and a capsid comprising SEQ ID NO: 5. In some embodiments, the AAV particle comprises a viral genome comprising SEQ ID NO: 2302 and a capsid encoded by a nucleic acid sequence comprising SEQ ID NO: 6. In some embodiments, the AAV particle comprises a viral genome comprising SEQ ID NO: 2302 and a capsid comprising SEQ ID NO: 4. In some embodiments, the AAV particle comprises a viral genome comprising SEQ ID NO: 2302 and a capsid comprising SEQ ID NO: 11. In some embodiments, the AAV particle comprises a viral genome comprising SEQ ID NO: 2302 and a capsid comprising SEQ ID NO: 2679. In some embodiments, the AAV particle comprises a viral genome comprising SEQ ID NO: 2302 and a capsid comprising SEQ ID NO: 2809. In some embodiments, the AAV particle comprises a viral genome comprising SEQ ID NO: 2302 and a capsid comprising SEQ ID NO: 2871.

In some embodiments, the AAV particle comprises a viral genome comprising SEQ ID NO: 2303 and a VOY101 capsid. In some embodiments, the AAV particle comprises a viral genome comprising SEQ ID NO: 2303 and a VOY201 capsid. In some embodiments, the AAV particle comprises a viral genome comprising SEQ ID NO: 2303 and an AAV9 capsid. In some embodiments, the AAV particle comprises a viral genome comprising SEQ ID NO: 2303 and an AAV9K449R capsid. In some embodiments, the AAV particle comprises a viral genome comprising SEQ ID NO: 2303 and an AAVPHP.B capsid. In some embodiments, the AAV particle comprises a viral genome comprising SEQ ID NO: 2303 and an AAVPHP.N capsid. In some embodiments, the AAV particle comprises a viral genome comprising SEQ ID NO: 2303 and an AAV2 variant capsid. In some embodiments, the AAV particle comprises a viral genome comprising SEQ ID NO: 2303 and an AAV2/3 variant capsid. In some embodiments, the AAV particle comprises a viral genome comprising SEQ ID NO: 2303 and a capsid comprising SEQ ID NO: 1. In some embodiments, the AAV particle comprises a viral genome comprising SEQ ID NO: 2303 and a capsid encoded by a nucleic acid sequence comprising SEQ ID NO: 2. In some embodiments, the AAV particle comprises a viral genome comprising SEQ ID NO: 2303 and a capsid comprising SEQ ID NO: 4534. In some embodiments, the AAV particle comprises a viral genome comprising SEQ ID NO: 2303 and a capsid encoded by a nucleic acid sequence comprising SEQ ID NO: 3. In some embodiments, the AAV particle comprises a viral genome comprising SEQ ID NO: 2303 and a capsid comprising SEQ ID NO: 138. In some embodiments, the AAV particle comprises a viral genome comprising SEQ ID NO: 2303 and a capsid encoded by a nucleic acid sequence comprising SEQ ID NO: 137. In some embodiments, the AAV particle comprises a viral genome comprising SEQ ID NO: 2303 and a capsid comprising SEQ ID NO: 5. In some embodiments, the AAV particle comprises a viral genome comprising SEQ ID NO: 2303 and a capsid encoded by a nucleic acid sequence comprising SEQ ID NO: 6. In some embodiments, the AAV particle comprises a viral genome comprising SEQ ID NO: 2303 and a capsid comprising SEQ ID NO: 4. In some embodiments, the AAV particle comprises a viral genome comprising SEQ ID NO: 2303 and a capsid comprising SEQ ID NO: 11. In some embodiments, the AAV particle comprises a viral genome comprising SEQ ID NO: 2303 and a capsid comprising SEQ ID NO: 2679. In some embodiments, the AAV particle comprises a viral genome comprising SEQ ID NO: 2303 and a capsid comprising SEQ ID NO: 2809. In some embodiments, the AAV particle comprises a viral genome comprising SEQ ID NO: 2303 and a capsid comprising SEQ ID NO: 2871.

In some embodiments, the AAV particle comprises a viral genome comprising SEQ ID NO: 2304 and a VOY101 capsid. In some embodiments, the AAV particle comprises a viral genome comprising SEQ ID NO: 2304 and a VOY201 capsid. In some embodiments, the AAV particle comprises a viral genome comprising SEQ ID NO: 2304 and an AAV9 capsid. In some embodiments, the AAV particle comprises a viral genome comprising SEQ ID NO: 2304 and an AAV9K449R capsid. In some embodiments, the AAV particle comprises a viral genome comprising SEQ ID NO: 2304 and an AAVPHP.B capsid. In some embodiments, the AAV particle comprises a viral genome comprising SEQ ID NO: 2304 and an AAVPHP.N capsid. In some embodiments, the AAV particle comprises a viral genome comprising SEQ ID NO: 2304 and an AAV2 variant capsid. In some embodiments, the AAV particle comprises a viral genome comprising SEQ ID NO: 2304 and an AAV2/3 variant capsid. In some embodiments, the AAV particle comprises a viral genome comprising SEQ ID NO: 2304 and a capsid comprising SEQ ID NO: 1. In some embodiments, the AAV particle comprises a viral genome comprising SEQ ID NO: 2304 and a capsid encoded by a nucleic acid sequence comprising SEQ ID NO: 2. In some embodiments, the AAV particle comprises a viral genome comprising SEQ ID NO: 2304 and a capsid comprising SEQ ID NO: 4534. In some embodiments, the AAV particle comprises a viral genome comprising SEQ ID NO: 2304 and a capsid encoded by a nucleic acid sequence comprising SEQ ID NO: 3. In some embodiments, the AAV particle comprises a viral genome comprising SEQ ID NO: 2304 and a capsid comprising SEQ ID NO: 138. In some embodiments, the AAV particle comprises a viral genome comprising SEQ ID NO: 2304 and a capsid encoded by a nucleic acid sequence comprising SEQ ID NO: 137. In some embodiments, the AAV particle comprises a viral genome comprising SEQ ID NO: 2304 and a capsid comprising SEQ ID NO: 5. In some embodiments, the AAV particle comprises a viral genome comprising SEQ ID NO: 2304 and a capsid encoded by a nucleic acid sequence comprising SEQ ID NO: 6. In some embodiments, the AAV particle comprises a viral genome comprising SEQ ID NO: 2304 and a capsid comprising SEQ ID NO: 4. In some embodiments, the AAV particle comprises a viral genome comprising SEQ ID NO: 2304 and a capsid comprising SEQ ID NO: 11. In some embodiments, the AAV particle comprises a viral genome comprising SEQ ID NO: 2304 and a capsid comprising SEQ ID NO: 2679. In some embodiments, the AAV particle comprises a viral genome comprising SEQ ID NO: 2304 and a capsid comprising SEQ ID NO: 2809. In some embodiments, the AAV particle comprises a viral genome comprising SEQ ID NO: 2304 and a capsid comprising SEQ ID NO: 2871.

In some embodiments, the AAV particle comprises a viral genome comprising SEQ ID NO: 2305 and a VOY101 capsid. In some embodiments, the AAV particle comprises a viral genome comprising SEQ ID NO: 2305 and a VOY201 capsid. In some embodiments, the AAV particle comprises a viral genome comprising SEQ ID NO: 2305 and an AAV9 capsid. In some embodiments, the AAV particle comprises a viral genome comprising SEQ ID NO: 2305 and an AAV9K449R capsid. In some embodiments, the AAV particle comprises a viral genome comprising SEQ ID NO: 2305 and an AAVPHP.B capsid. In some embodiments, the AAV particle comprises a viral genome comprising SEQ ID NO: 2305 and an AAVPHP.N capsid. In some embodiments, the AAV particle comprises a viral genome comprising SEQ ID NO: 2305 and an AAV2 variant capsid. In some embodiments, the AAV particle comprises a viral genome comprising SEQ ID NO: 2305 and an AAV2/3 variant capsid. In some embodiments, the AAV particle comprises a viral genome comprising SEQ ID NO: 2305 and a capsid comprising SEQ ID NO: 1. In some embodiments, the AAV particle comprises a viral genome comprising SEQ ID NO: 2305 and a capsid encoded by a nucleic acid sequence comprising SEQ ID NO: 2. In some embodiments, the AAV particle comprises a viral genome comprising SEQ ID NO: 2305 and a capsid comprising SEQ ID NO: 4534. In some embodiments, the AAV particle comprises a viral genome comprising SEQ ID NO: 2305 and a capsid encoded by a nucleic acid sequence comprising SEQ ID NO: 3. In some embodiments, the AAV particle comprises a viral genome comprising SEQ ID NO: 2305 and a capsid comprising SEQ ID NO: 138. In some embodiments, the AAV particle comprises a viral genome comprising SEQ ID NO: 2305 and a capsid encoded by a nucleic acid sequence comprising SEQ ID NO: 137. In some embodiments, the AAV particle comprises a viral genome comprising SEQ ID NO: 2305 and a capsid comprising SEQ ID NO: 5. In some embodiments, the AAV particle comprises a viral genome comprising SEQ ID NO: 2305 and a capsid encoded by a nucleic acid sequence comprising SEQ ID NO: 6. In some embodiments, the AAV particle comprises a viral genome comprising SEQ ID NO: 2305 and a capsid comprising SEQ ID NO: 4. In some embodiments, the AAV particle comprises a viral genome comprising SEQ ID NO: 2305 and a capsid comprising SEQ ID NO: 11. In some embodiments, the AAV particle comprises a viral genome comprising SEQ ID NO: 2305 and a capsid comprising SEQ ID NO: 2679. In some embodiments, the AAV particle comprises a viral genome comprising SEQ ID NO: 2305 and a capsid comprising SEQ ID NO: 2809. In some embodiments, the AAV particle comprises a viral genome comprising SEQ ID NO: 2305 and a capsid comprising SEQ ID NO: 2871.

In some embodiments, the AAV particle comprises a viral genome comprising SEQ ID NO: 2306 and a VOY101 capsid. In some embodiments, the AAV particle comprises a viral genome comprising SEQ ID NO: 2306 and a VOY201 capsid. In some embodiments, the AAV particle comprises a viral genome comprising SEQ ID NO: 2306 and an AAV9 capsid. In some embodiments, the AAV particle comprises a viral genome comprising SEQ ID NO: 2306 and an AAV9K449R capsid. In some embodiments, the AAV particle comprises a viral genome comprising SEQ ID NO: 2306 and an AAVPHP.B capsid. In some embodiments, the AAV particle comprises a viral genome comprising SEQ ID NO: 2306 and an AAVPHP.N capsid. In some embodiments, the AAV particle comprises a viral genome comprising SEQ ID NO: 2306 and an AAV2 variant capsid. In some embodiments, the AAV particle comprises a viral genome comprising SEQ ID NO: 2306 and an AAV2/3 variant capsid. In some embodiments, the AAV particle comprises a viral genome comprising SEQ ID NO: 2306 and a capsid comprising SEQ ID NO: 1. In some embodiments, the AAV particle comprises a viral genome comprising SEQ ID NO: 2306 and a capsid encoded by a nucleic acid sequence comprising SEQ ID NO: 2. In some embodiments, the AAV particle comprises a viral genome comprising SEQ ID NO: 2306 and a capsid comprising SEQ ID NO: 4534. In some embodiments, the AAV particle comprises a viral genome comprising SEQ ID NO: 2306 and a capsid encoded by a nucleic acid sequence comprising SEQ ID NO: 3. In some embodiments, the AAV particle comprises a viral genome comprising SEQ ID NO: 2306 and a capsid comprising SEQ ID NO: 138. In some embodiments, the AAV particle comprises a viral genome comprising SEQ ID NO: 2306 and a capsid encoded by a nucleic acid sequence comprising SEQ ID NO: 137. In some embodiments, the AAV particle comprises a viral genome comprising SEQ ID NO: 2306 and a capsid comprising SEQ ID NO: 5. In some embodiments, the AAV particle comprises a viral genome comprising SEQ ID NO: 2306 and a capsid encoded by a nucleic acid sequence comprising SEQ ID NO: 6. In some embodiments, the AAV particle comprises a viral genome comprising SEQ ID NO: 2306 and a capsid comprising SEQ ID NO: 4. In some embodiments, the AAV particle comprises a viral genome comprising SEQ ID NO: 2306 and a capsid comprising SEQ ID NO: 11. In some embodiments, the AAV particle comprises a viral genome comprising SEQ ID NO: 2306 and a capsid comprising SEQ ID NO: 2679. In some embodiments, the AAV particle comprises a viral genome comprising SEQ ID NO: 2306 and a capsid comprising SEQ ID NO: 2809. In some embodiments, the AAV particle comprises a viral genome comprising SEQ ID NO: 2306 and a capsid comprising SEQ ID NO: 2871.

In some embodiments, the AAV particle comprises a viral genome comprising SEQ ID NO: 2307 and a VOY101 capsid. In some embodiments, the AAV particle comprises a viral genome comprising SEQ ID NO: 2307 and a VOY201 capsid. In some embodiments, the AAV particle comprises a viral genome comprising SEQ ID NO: 2307 and an AAV9 capsid. In some embodiments, the AAV particle comprises a viral genome comprising SEQ ID NO: 2307 and an AAV9K449R capsid. In some embodiments, the AAV particle comprises a viral genome comprising SEQ ID NO: 2307 and an AAVPHP.B capsid. In some embodiments, the AAV particle comprises a viral genome comprising SEQ ID NO: 2307 and an AAVPHP.N capsid. In some embodiments, the AAV particle comprises a viral genome comprising SEQ ID NO: 2307 and an AAV2 variant capsid. In some embodiments, the AAV particle comprises a viral genome comprising SEQ ID NO: 2307 and an AAV2/3 variant capsid. In some embodiments, the AAV particle comprises a viral genome comprising SEQ ID NO: 2307 and a capsid comprising SEQ ID NO: 1. In some embodiments, the AAV particle comprises a viral genome comprising SEQ ID NO: 2307 and a capsid encoded by a nucleic acid sequence comprising SEQ ID NO: 2. In some embodiments, the AAV particle comprises a viral genome comprising SEQ ID NO: 2307 and a capsid comprising SEQ ID NO: 4534. In some embodiments, the AAV particle comprises a viral genome comprising SEQ ID NO: 2307 and a capsid encoded by a nucleic acid sequence comprising SEQ ID NO: 3. In some embodiments, the AAV particle comprises a viral genome comprising SEQ ID NO: 2307 and a capsid comprising SEQ ID NO: 138. In some embodiments, the AAV particle comprises a viral genome comprising SEQ ID NO: 2307 and a capsid encoded by a nucleic acid sequence comprising SEQ ID NO: 137. In some embodiments, the AAV particle comprises a viral genome comprising SEQ ID NO: 2307 and a capsid comprising SEQ ID NO: 5. In some embodiments, the AAV particle comprises a viral genome comprising SEQ ID NO: 2307 and a capsid encoded by a nucleic acid sequence comprising SEQ ID NO: 6. In some embodiments, the AAV particle comprises a viral genome comprising SEQ ID NO: 2307 and a capsid comprising SEQ ID NO: 4. In some embodiments, the AAV particle comprises a viral genome comprising SEQ ID NO: 2307 and a capsid comprising SEQ ID NO: 11. In some embodiments, the AAV particle comprises a viral genome comprising SEQ ID NO: 2307 and a capsid comprising SEQ ID NO: 2679. In some embodiments, the AAV particle comprises a viral genome comprising SEQ ID NO: 2307 and a capsid comprising SEQ ID NO: 2809. In some embodiments, the AAV particle comprises a viral genome comprising SEQ ID NO: 2307 and a capsid comprising SEQ ID NO: 2871.

In some embodiments, the AAV particle comprises a viral genome comprising SEQ ID NO: 2308 and a VOY101 capsid. In some embodiments, the AAV particle comprises a viral genome comprising SEQ ID NO: 2308 and a VOY201 capsid. In some embodiments, the AAV particle comprises a viral genome comprising SEQ ID NO: 2308 and an AAV9 capsid. In some embodiments, the AAV particle comprises a viral genome comprising SEQ ID NO: 2308 and an AAV9K449R capsid. In some embodiments, the AAV particle comprises a viral genome comprising SEQ ID NO: 2308 and an AAVPHP.B capsid. In some embodiments, the AAV particle comprises a viral genome comprising SEQ ID NO: 2308 and an AAVPHP.N capsid. In some embodiments, the AAV particle comprises a viral genome comprising SEQ ID NO: 2308 and an AAV2 variant capsid. In some embodiments, the AAV particle comprises a viral genome comprising SEQ ID NO: 2308 and an AAV2/3 variant capsid. In some embodiments, the AAV particle comprises a viral genome comprising SEQ ID NO: 2308 and a capsid comprising SEQ ID NO: 1. In some embodiments, the AAV particle comprises a viral genome comprising SEQ ID NO: 2308 and a capsid encoded by a nucleic acid sequence comprising SEQ ID NO: 2. In some embodiments, the AAV particle comprises a viral genome comprising SEQ ID NO: 2308 and a capsid comprising SEQ ID NO: 4534. In some embodiments, the AAV particle comprises a viral genome comprising SEQ ID NO: 2308 and a capsid encoded by a nucleic acid sequence comprising SEQ ID NO: 3. In some embodiments, the AAV particle comprises a viral genome comprising SEQ ID NO: 2308 and a capsid comprising SEQ ID NO: 138. In some embodiments, the AAV particle comprises a viral genome comprising SEQ ID NO: 2308 and a capsid encoded by a nucleic acid sequence comprising SEQ ID NO: 137. In some embodiments, the AAV particle comprises a viral genome comprising SEQ ID NO: 2308 and a capsid comprising SEQ ID NO: 5. In some embodiments, the AAV particle comprises a viral genome comprising SEQ ID NO: 2308 and a capsid encoded by a nucleic acid sequence comprising SEQ ID NO: 6. In some embodiments, the AAV particle comprises a viral genome comprising SEQ ID NO: 2308 and a capsid comprising SEQ ID NO: 4. In some embodiments, the AAV particle comprises a viral genome comprising SEQ ID NO: 2308 and a capsid comprising SEQ ID NO: 11. In some embodiments, the AAV particle comprises a viral genome comprising SEQ ID NO: 2308 and a capsid comprising SEQ ID NO: 2679. In some embodiments, the AAV particle comprises a viral genome comprising SEQ ID NO: 2308 and a capsid comprising SEQ ID NO: 2809. In some embodiments, the AAV particle comprises a viral genome comprising SEQ ID NO: 2308 and a capsid comprising SEQ ID NO: 2871.

In some embodiments, the AAV particle comprises a viral genome comprising SEQ ID NO: 2309 and a VOY101 capsid. In some embodiments, the AAV particle comprises a viral genome comprising SEQ ID NO: 2309 and a VOY201 capsid. In some embodiments, the AAV particle comprises a viral genome comprising SEQ ID NO: 2309 and an AAV9 capsid. In some embodiments, the AAV particle comprises a viral genome comprising SEQ ID NO: 2309 and an AAV9K449R capsid. In some embodiments, the AAV particle comprises a viral genome comprising SEQ ID NO: 2309 and an AAVPHP.B capsid. In some embodiments, the AAV particle comprises a viral genome comprising SEQ ID NO: 2309 and an AAVPHP.N capsid. In some embodiments, the AAV particle comprises a viral genome comprising SEQ ID NO: 2309 and an AAV2 variant capsid. In some embodiments, the AAV particle comprises a viral genome comprising SEQ ID NO: 2309 and an AAV2/3 variant capsid. In some embodiments, the AAV particle comprises a viral genome comprising SEQ ID NO: 2309 and a capsid comprising SEQ ID NO: 1. In some embodiments, the AAV particle comprises a viral genome comprising SEQ ID NO: 2309 and a capsid encoded by a nucleic acid sequence comprising SEQ ID NO: 2. In some embodiments, the AAV particle comprises a viral genome comprising SEQ ID NO: 2309 and a capsid comprising SEQ ID NO: 4534. In some embodiments, the AAV particle comprises a viral genome comprising SEQ ID NO: 2309 and a capsid encoded by a nucleic acid sequence comprising SEQ ID NO: 3. In some embodiments, the AAV particle comprises a viral genome comprising SEQ ID NO: 2309 and a capsid comprising SEQ ID NO: 138. In some embodiments, the AAV particle comprises a viral genome comprising SEQ ID NO: 2309 and a capsid encoded by a nucleic acid sequence comprising SEQ ID NO: 137. In some embodiments, the AAV particle comprises a viral genome comprising SEQ ID NO: 2309 and a capsid comprising SEQ ID NO: 5. In some embodiments, the AAV particle comprises a viral genome comprising SEQ ID NO: 2309 and a capsid encoded by a nucleic acid sequence comprising SEQ ID NO: 6. In some embodiments, the AAV particle comprises a viral genome comprising SEQ ID NO: 2309 and a capsid comprising SEQ ID NO: 4. In some embodiments, the AAV particle comprises a viral genome comprising SEQ ID NO: 2309 and a capsid comprising SEQ ID NO: 11. In some embodiments, the AAV particle comprises a viral genome comprising SEQ ID NO: 2309 and a capsid comprising SEQ ID NO: 2679. In some embodiments, the AAV particle comprises a viral genome comprising SEQ ID NO: 2309 and a capsid comprising SEQ ID NO: 2809. In some embodiments, the AAV particle comprises a viral genome comprising SEQ ID NO: 2309 and a capsid comprising SEQ ID NO: 2871.

In some embodiments, the AAV particle comprises a viral genome comprising SEQ ID NO: 2310 and a VOY101 capsid. In some embodiments, the AAV particle comprises a viral genome comprising SEQ ID NO: 2310 and a VOY201 capsid. In some embodiments, the AAV particle comprises a viral genome comprising SEQ ID NO: 2310 and an AAV9 capsid. In some embodiments, the AAV particle comprises a viral genome comprising SEQ ID NO: 2310 and an AAV9K449R capsid. In some embodiments, the AAV particle comprises a viral genome comprising SEQ ID NO: 2310 and an AAVPHP.B capsid. In some embodiments, the AAV particle comprises a viral genome comprising SEQ ID NO: 2310 and an AAVPHP.N capsid. In some embodiments, the AAV particle comprises a viral genome comprising SEQ ID NO: 2310 and an AAV2 variant capsid. In some embodiments, the AAV particle comprises a viral genome comprising SEQ ID NO: 2310 and an AAV2/3 variant capsid. In some embodiments, the AAV particle comprises a viral genome comprising SEQ ID NO: 2310 and a capsid comprising SEQ ID NO: 1. In some embodiments, the AAV particle comprises a viral genome comprising SEQ ID NO: 2310 and a capsid encoded by a nucleic acid sequence comprising SEQ ID NO: 2. In some embodiments, the AAV particle comprises a viral genome comprising SEQ ID NO: 2310 and a capsid comprising SEQ ID NO: 4534. In some embodiments, the AAV particle comprises a viral genome comprising SEQ ID NO: 2310 and a capsid encoded by a nucleic acid sequence comprising SEQ ID NO: 3. In some embodiments, the AAV particle comprises a viral genome comprising SEQ ID NO: 2310 and a capsid comprising SEQ ID NO: 138. In some embodiments, the AAV particle comprises a viral genome comprising SEQ ID NO: 2310 and a capsid encoded by a nucleic acid sequence comprising SEQ ID NO: 137. In some embodiments, the AAV particle comprises a viral genome comprising SEQ ID NO: 2310 and a capsid comprising SEQ ID NO: 5. In some embodiments, the AAV particle comprises a viral genome comprising SEQ ID NO: 2310 and a capsid encoded by a nucleic acid sequence comprising SEQ ID NO: 6. In some embodiments, the AAV particle comprises a viral genome comprising SEQ ID NO: 2310 and a capsid comprising SEQ ID NO: 4. In some embodiments, the AAV particle comprises a viral genome comprising SEQ ID NO: 2310 and a capsid comprising SEQ ID NO: 11. In some embodiments, the AAV particle comprises a viral genome comprising SEQ ID NO: 2310 and a capsid comprising SEQ ID NO: 2679. In some embodiments, the AAV particle comprises a viral genome comprising SEQ ID NO: 2310 and a capsid comprising SEQ ID NO: 2809. In some embodiments, the AAV particle comprises a viral genome comprising SEQ ID NO: 2310 and a capsid comprising SEQ ID NO: 2871.

In some embodiments, the AAV particle comprises a viral genome comprising SEQ ID NO: 2311 and a VOY101 capsid. In some embodiments, the AAV particle comprises a viral genome comprising SEQ ID NO: 2311 and a VOY201 capsid. In some embodiments, the AAV particle comprises a viral genome comprising SEQ ID NO: 2311 and an AAV9 capsid. In some embodiments, the AAV particle comprises a viral genome comprising SEQ ID NO: 2311 and an AAV9K449R capsid. In some embodiments, the AAV particle comprises a viral genome comprising SEQ ID NO: 2311 and an AAVPHP.B capsid. In some embodiments, the AAV particle comprises a viral genome comprising SEQ ID NO: 2311 and an AAVPHP.N capsid. In some embodiments, the AAV particle comprises a viral genome comprising SEQ ID NO: 2311 and an AAV2 variant capsid. In some embodiments, the AAV particle comprises a viral genome comprising SEQ ID NO: 2311 and an AAV2/3 variant capsid. In some embodiments, the AAV particle comprises a viral genome comprising SEQ ID NO: 2311 and a capsid comprising SEQ ID NO: 1. In some embodiments, the AAV particle comprises a viral genome comprising SEQ ID NO: 2311 and a capsid encoded by a nucleic acid sequence comprising SEQ ID NO: 2. In some embodiments, the AAV particle comprises a viral genome comprising SEQ ID NO: 2311 and a capsid comprising SEQ ID NO: 4534. In some embodiments, the AAV particle comprises a viral genome comprising SEQ ID NO: 2311 and a capsid encoded by a nucleic acid sequence comprising SEQ ID NO: 3. In some embodiments, the AAV particle comprises a viral genome comprising SEQ ID NO: 2311 and a capsid comprising SEQ ID NO: 138. In some embodiments, the AAV particle comprises a viral genome comprising SEQ ID NO: 2311 and a capsid encoded by a nucleic acid sequence comprising SEQ ID NO: 137. In some embodiments, the AAV particle comprises a viral genome comprising SEQ ID NO: 2311 and a capsid comprising SEQ ID NO: 5. In some embodiments, the AAV particle comprises a viral genome comprising SEQ ID NO: 2311 and a capsid encoded by a nucleic acid sequence comprising SEQ ID NO: 6. In some embodiments, the AAV particle comprises a viral genome comprising SEQ ID NO: 2311 and a capsid comprising SEQ ID NO: 4. In some embodiments, the AAV particle comprises a viral genome comprising SEQ ID NO: 2311 and a capsid comprising SEQ ID NO: 11. In some embodiments, the AAV particle comprises a viral genome comprising SEQ ID NO: 2311 and a capsid comprising SEQ ID NO: 2679. In some embodiments, the AAV particle comprises a viral genome comprising SEQ ID NO: 2311 and a capsid comprising SEQ ID NO: 2809. In some embodiments, the AAV particle comprises a viral genome comprising SEQ ID NO: 2311 and a capsid comprising SEQ ID NO: 2871.

In some embodiments, the AAV particle comprises a viral genome comprising SEQ ID NO: 2312 and a VOY101 capsid. In some embodiments, the AAV particle comprises a viral genome comprising SEQ ID NO: 2312 and a VOY201 capsid. In some embodiments, the AAV particle comprises a viral genome comprising SEQ ID NO: 2312 and an AAV9 capsid. In some embodiments, the AAV particle comprises a viral genome comprising SEQ ID NO: 2312 and an AAV9K449R capsid. In some embodiments, the AAV particle comprises a viral genome comprising SEQ ID NO: 2312 and an AAVPHP.B capsid. In some embodiments, the AAV particle comprises a viral genome comprising SEQ ID NO: 2312 and an AAVPHP.N capsid. In some embodiments, the AAV particle comprises a viral genome comprising SEQ ID NO: 2312 and an AAV2 variant capsid. In some embodiments, the AAV particle comprises a viral genome comprising SEQ ID NO: 2312 and an AAV2/3 variant capsid. In some embodiments, the AAV particle comprises a viral genome comprising SEQ ID NO: 2312 and a capsid comprising SEQ ID NO: 1. In some embodiments, the AAV particle comprises a viral genome comprising SEQ ID NO: 2312 and a capsid encoded by a nucleic acid sequence comprising SEQ ID NO: 2. In some embodiments, the AAV particle comprises a viral genome comprising SEQ ID NO: 2312 and a capsid comprising SEQ ID NO: 4534. In some embodiments, the AAV particle comprises a viral genome comprising SEQ ID NO: 2312 and a capsid encoded by a nucleic acid sequence comprising SEQ ID NO: 3. In some embodiments, the AAV particle comprises a viral genome comprising SEQ ID NO: 2312 and a capsid comprising SEQ ID NO: 138. In some embodiments, the AAV particle comprises a viral genome comprising SEQ ID NO: 2312 and a capsid encoded by a nucleic acid sequence comprising SEQ ID NO: 137. In some embodiments, the AAV particle comprises a viral genome comprising SEQ ID NO: 2312 and a capsid comprising SEQ ID NO: 5. In some embodiments, the AAV particle comprises a viral genome comprising SEQ ID NO: 2312 and a capsid encoded by a nucleic acid sequence comprising SEQ ID NO: 6. In some embodiments, the AAV particle comprises a viral genome comprising SEQ ID NO: 2312 and a capsid comprising SEQ ID NO: 4. In some embodiments, the AAV particle comprises a viral genome comprising SEQ ID NO: 2312 and a capsid comprising SEQ ID NO: 11. In some embodiments, the AAV particle comprises a viral genome comprising SEQ ID NO: 2312 and a capsid comprising SEQ ID NO: 2679. In some embodiments, the AAV particle comprises a viral genome comprising SEQ ID NO: 2312 and a capsid comprising SEQ ID NO: 2809. In some embodiments, the AAV particle comprises a viral genome comprising SEQ ID NO: 2312 and a capsid comprising SEQ ID NO: 2871.

In some embodiments, the AAV particle comprises a viral genome comprising SEQ ID NO: 2313 and a VOY101 capsid. In some embodiments, the AAV particle comprises a viral genome comprising SEQ ID NO: 2313 and a VOY201 capsid. In some embodiments, the AAV particle comprises a viral genome comprising SEQ ID NO: 2313 and an AAV9 capsid. In some embodiments, the AAV particle comprises a viral genome comprising SEQ ID NO: 2313 and an AAV9K449R capsid. In some embodiments, the AAV particle comprises a viral genome comprising SEQ ID NO: 2313 and an AAVPHP.B capsid. In some embodiments, the AAV particle comprises a viral genome comprising SEQ ID NO: 2313 and an AAVPHP.N capsid. In some embodiments, the AAV particle comprises a viral genome comprising SEQ ID NO: 2313 and an AAV2 variant capsid. In some embodiments, the AAV particle comprises a viral genome comprising SEQ ID NO: 2313 and an AAV2/3 variant capsid. In some embodiments, the AAV particle comprises a viral genome comprising SEQ ID NO: 2313 and a capsid comprising SEQ ID NO: 1. In some embodiments, the AAV particle comprises a viral genome comprising SEQ ID NO: 2313 and a capsid encoded by a nucleic acid sequence comprising SEQ ID NO: 2. In some embodiments, the AAV particle comprises a viral genome comprising SEQ ID NO: 2313 and a capsid comprising SEQ ID NO: 4534. In some embodiments, the AAV particle comprises a viral genome comprising SEQ ID NO: 2313 and a capsid encoded by a nucleic acid sequence comprising SEQ ID NO: 3. In some embodiments, the AAV particle comprises a viral genome comprising SEQ ID NO: 2313 and a capsid comprising SEQ ID NO: 138. In some embodiments, the AAV particle comprises a viral genome comprising SEQ ID NO: 2313 and a capsid encoded by a nucleic acid sequence comprising SEQ ID NO: 137. In some embodiments, the AAV particle comprises a viral genome comprising SEQ ID NO: 2313 and a capsid comprising SEQ ID NO: 5. In some embodiments, the AAV particle comprises a viral genome comprising SEQ ID NO: 2313 and a capsid encoded by a nucleic acid sequence comprising SEQ ID NO: 6. In some embodiments, the AAV particle comprises a viral genome comprising SEQ ID NO: 2313 and a capsid comprising SEQ ID NO: 4. In some embodiments, the AAV particle comprises a viral genome comprising SEQ ID NO: 2313 and a capsid comprising SEQ ID NO: 11. In some embodiments, the AAV particle comprises a viral genome comprising SEQ ID NO: 2313 and a capsid comprising SEQ ID NO: 2679. In some embodiments, the AAV particle comprises a viral genome comprising SEQ ID NO: 2313 and a capsid comprising SEQ ID NO: 2809. In some embodiments, the AAV particle comprises a viral genome comprising SEQ ID NO: 2313 and a capsid comprising SEQ ID NO: 2871.

In some embodiments, the AAV particle comprises a viral genome comprising SEQ ID NO: 2314 and a VOY101 capsid. In some embodiments, the AAV particle comprises a viral genome comprising SEQ ID NO: 2314 and a VOY201 capsid. In some embodiments, the AAV particle comprises a viral genome comprising SEQ ID NO: 2314 and an AAV9 capsid. In some embodiments, the AAV particle comprises a viral genome comprising SEQ ID NO: 2314 and an AAV9K449R capsid. In some embodiments, the AAV particle comprises a viral genome comprising SEQ ID NO: 2314 and an AAVPHP.B capsid. In some embodiments, the AAV particle comprises a viral genome comprising SEQ ID NO: 2314 and an AAVPHP.N capsid. In some embodiments, the AAV particle comprises a viral genome comprising SEQ ID NO: 2314 and an AAV2 variant capsid. In some embodiments, the AAV particle comprises a viral genome comprising SEQ ID NO: 2314 and an AAV2/3 variant capsid. In some embodiments, the AAV particle comprises a viral genome comprising SEQ ID NO: 2314 and a capsid comprising SEQ ID NO: 1. In some embodiments, the AAV particle comprises a viral genome comprising SEQ ID NO: 2314 and a capsid encoded by a nucleic acid sequence comprising SEQ ID NO: 2. In some embodiments, the AAV particle comprises a viral genome comprising SEQ ID NO: 2314 and a capsid comprising SEQ ID NO: 4534. In some embodiments, the AAV particle comprises a viral genome comprising SEQ ID NO: 2314 and a capsid encoded by a nucleic acid sequence comprising SEQ ID NO: 3. In some embodiments, the AAV particle comprises a viral genome comprising SEQ ID NO: 2314 and a capsid comprising SEQ ID NO: 138. In some embodiments, the AAV particle comprises a viral genome comprising SEQ ID NO: 2314 and a capsid encoded by a nucleic acid sequence comprising SEQ ID NO: 137. In some embodiments, the AAV particle comprises a viral genome comprising SEQ ID NO: 2314 and a capsid comprising SEQ ID NO: 5. In some embodiments, the AAV particle comprises a viral genome comprising SEQ ID NO: 2314 and a capsid encoded by a nucleic acid sequence comprising SEQ ID NO: 6. In some embodiments, the AAV particle comprises a viral genome comprising SEQ ID NO: 2314 and a capsid comprising SEQ ID NO: 4. In some embodiments, the AAV particle comprises a viral genome comprising SEQ ID NO: 2314 and a capsid comprising SEQ ID NO: 11. In some embodiments, the AAV particle comprises a viral genome comprising SEQ ID NO: 2314 and a capsid comprising SEQ ID NO: 2679. In some embodiments, the AAV particle comprises a viral genome comprising SEQ ID NO: 2314 and a capsid comprising SEQ ID NO: 2809. In some embodiments, the AAV particle comprises a viral genome comprising SEQ ID NO: 2314 and a capsid comprising SEQ ID NO: 2871.

In some embodiments, the AAV particle comprises a viral genome comprising SEQ ID NO: 2315 and a VOY101 capsid. In some embodiments, the AAV particle comprises a viral genome comprising SEQ ID NO: 2315 and a VOY201 capsid. In some embodiments, the AAV particle comprises a viral genome comprising SEQ ID NO: 2315 and an AAV9 capsid. In some embodiments, the AAV particle comprises a viral genome comprising SEQ ID NO: 2315 and an AAV9K449R capsid. In some embodiments, the AAV particle comprises a viral genome comprising SEQ ID NO: 2315 and an AAVPHP.B capsid. In some embodiments, the AAV particle comprises a viral genome comprising SEQ ID NO: 2315 and an AAVPHP.N capsid. In some embodiments, the AAV particle comprises a viral genome comprising SEQ ID NO: 2315 and an AAV2 variant capsid. In some embodiments, the AAV particle comprises a viral genome comprising SEQ ID NO: 2315 and an AAV2/3 variant capsid. In some embodiments, the AAV particle comprises a viral genome comprising SEQ ID NO: 2315 and a capsid comprising SEQ ID NO: 1. In some embodiments, the AAV particle comprises a viral genome comprising SEQ ID NO: 2315 and a capsid encoded by a nucleic acid sequence comprising SEQ ID NO: 2. In some embodiments, the AAV particle comprises a viral genome comprising SEQ ID NO: 2315 and a capsid comprising SEQ ID NO: 4534. In some embodiments, the AAV particle comprises a viral genome comprising SEQ ID NO: 2315 and a capsid encoded by a nucleic acid sequence comprising SEQ ID NO: 3. In some embodiments, the AAV particle comprises a viral genome comprising SEQ ID NO: 2315 and a capsid comprising SEQ ID NO: 138. In some embodiments, the AAV particle comprises a viral genome comprising SEQ ID NO: 2315 and a capsid encoded by a nucleic acid sequence comprising SEQ ID NO: 137. In some embodiments, the AAV particle comprises a viral genome comprising SEQ ID NO: 2315 and a capsid comprising SEQ ID NO: 5. In some embodiments, the AAV particle comprises a viral genome comprising SEQ ID NO: 2315 and a capsid encoded by a nucleic acid sequence comprising SEQ ID NO: 6. In some embodiments, the AAV particle comprises a viral genome comprising SEQ ID NO: 2315 and a capsid comprising SEQ ID NO: 4. In some embodiments, the AAV particle comprises a viral genome comprising SEQ ID NO: 2315 and a capsid comprising SEQ ID NO: 11. In some embodiments, the AAV particle comprises a viral genome comprising SEQ ID NO: 2315 and a capsid comprising SEQ ID NO: 2679. In some embodiments, the AAV particle comprises a viral genome comprising SEQ ID NO: 2315 and a capsid comprising SEQ ID NO: 2809. In some embodiments, the AAV particle comprises a viral genome comprising SEQ ID NO: 2315 and a capsid comprising SEQ ID NO: 2871.

In some embodiments, the AAV particle comprises a viral genome comprising SEQ ID NO: 2316 and a VOY101 capsid. In some embodiments, the AAV particle comprises a viral genome comprising SEQ ID NO: 2316 and a VOY201 capsid. In some embodiments, the AAV particle comprises a viral genome comprising SEQ ID NO: 2316 and an AAV9 capsid. In some embodiments, the AAV particle comprises a viral genome comprising SEQ ID NO: 2316 and an AAV9K449R capsid. In some embodiments, the AAV particle comprises a viral genome comprising SEQ ID NO: 2316 and an AAVPHP.B capsid. In some embodiments, the AAV particle comprises a viral genome comprising SEQ ID NO: 2316 and an AAVPHP.N capsid. In some embodiments, the AAV particle comprises a viral genome comprising SEQ ID NO: 2316 and an AAV2 variant capsid. In some embodiments, the AAV particle comprises a viral genome comprising SEQ ID NO: 2316 and an AAV2/3 variant capsid. In some embodiments, the AAV particle comprises a viral genome comprising SEQ ID NO: 2316 and a capsid comprising SEQ ID NO: 1. In some embodiments, the AAV particle comprises a viral genome comprising SEQ ID NO: 2316 and a capsid encoded by a nucleic acid sequence comprising SEQ ID NO: 2. In some embodiments, the AAV particle comprises a viral genome comprising SEQ ID NO: 2316 and a capsid comprising SEQ ID NO: 4534. In some embodiments, the AAV particle comprises a viral genome comprising SEQ ID NO: 2316 and a capsid encoded by a nucleic acid sequence comprising SEQ ID NO: 3. In some embodiments, the AAV particle comprises a viral genome comprising SEQ ID NO: 2316 and a capsid comprising SEQ ID NO: 138. In some embodiments, the AAV particle comprises a viral genome comprising SEQ ID NO: 2316 and a capsid encoded by a nucleic acid sequence comprising SEQ ID NO: 137. In some embodiments, the AAV particle comprises a viral genome comprising SEQ ID NO: 2316 and a capsid comprising SEQ ID NO: 5. In some embodiments, the AAV particle comprises a viral genome comprising SEQ ID NO: 2316 and a capsid encoded by a nucleic acid sequence comprising SEQ ID NO: 6. In some embodiments, the AAV particle comprises a viral genome comprising SEQ ID NO: 2316 and a capsid comprising SEQ ID NO: 4. In some embodiments, the AAV particle comprises a viral genome comprising SEQ ID NO: 2316 and a capsid comprising SEQ ID NO: 11. In some embodiments, the AAV particle comprises a viral genome comprising SEQ ID NO: 2316 and a capsid comprising SEQ ID NO: 2679. In some embodiments, the AAV particle comprises a viral genome comprising SEQ ID NO: 2316 and a capsid comprising SEQ ID NO: 2809. In some embodiments, the AAV particle comprises a viral genome comprising SEQ ID NO: 2316 and a capsid comprising SEQ ID NO: 2871.

In some embodiments, the AAV particle comprises a viral genome comprising SEQ ID NO: 2317 and a VOY101 capsid. In some embodiments, the AAV particle comprises a viral genome comprising SEQ ID NO: 2317 and a VOY201 capsid. In some embodiments, the AAV particle comprises a viral genome comprising SEQ ID NO: 2317 and an AAV9 capsid. In some embodiments, the AAV particle comprises a viral genome comprising SEQ ID NO: 2317 and an AAV9K449R capsid. In some embodiments, the AAV particle comprises a viral genome comprising SEQ ID NO: 2317 and an AAVPHP.B capsid. In some embodiments, the AAV particle comprises a viral genome comprising SEQ ID NO: 2317 and an AAVPHP.N capsid. In some embodiments, the AAV particle comprises a viral genome comprising SEQ ID NO: 2317 and an AAV2 variant capsid. In some embodiments, the AAV particle comprises a viral genome comprising SEQ ID NO: 2317 and an AAV2/3 variant capsid. In some embodiments, the AAV particle comprises a viral genome comprising SEQ ID NO: 2317 and a capsid comprising SEQ ID NO: 1. In some embodiments, the AAV particle comprises a viral genome comprising SEQ ID NO: 2317 and a capsid encoded by a nucleic acid sequence comprising SEQ ID NO: 2. In some embodiments, the AAV particle comprises a viral genome comprising SEQ ID NO: 2317 and a capsid comprising SEQ ID NO: 4534. In some embodiments, the AAV particle comprises a viral genome comprising SEQ ID NO: 2317 and a capsid encoded by a nucleic acid sequence comprising SEQ ID NO: 3. In some embodiments, the AAV particle comprises a viral genome comprising SEQ ID NO: 2317 and a capsid comprising SEQ ID NO: 138. In some embodiments, the AAV particle comprises a viral genome comprising SEQ ID NO: 2317 and a capsid encoded by a nucleic acid sequence comprising SEQ ID NO: 137. In some embodiments, the AAV particle comprises a viral genome comprising SEQ ID NO: 2317 and a capsid comprising SEQ ID NO: 5. In some embodiments, the AAV particle comprises a viral genome comprising SEQ ID NO: 2317 and a capsid encoded by a nucleic acid sequence comprising SEQ ID NO: 6. In some embodiments, the AAV particle comprises a viral genome comprising SEQ ID NO: 2317 and a capsid comprising SEQ ID NO: 4. In some embodiments, the AAV particle comprises a viral genome comprising SEQ ID NO: 2317 and a capsid comprising SEQ ID NO: 11. In some embodiments, the AAV particle comprises a viral genome comprising SEQ ID NO: 2317 and a capsid comprising SEQ ID NO: 2679. In some embodiments, the AAV particle comprises a viral genome comprising SEQ ID NO: 2317 and a capsid comprising SEQ ID NO: 2809. In some embodiments, the AAV particle comprises a viral genome comprising SEQ ID NO: 2317 and a capsid comprising SEQ ID NO: 2871.

In some embodiments, the AAV particle comprises a viral genome comprising SEQ ID NO: 2318 and a VOY101 capsid. In some embodiments, the AAV particle comprises a viral genome comprising SEQ ID NO: 2318 and a VOY201 capsid. In some embodiments, the AAV particle comprises a viral genome comprising SEQ ID NO: 2318 and an AAV9 capsid. In some embodiments, the AAV particle comprises a viral genome comprising SEQ ID NO: 2318 and an AAV9K449R capsid. In some embodiments, the AAV particle comprises a viral genome comprising SEQ ID NO: 2318 and an AAVPHP.B capsid. In some embodiments, the AAV particle comprises a viral genome comprising SEQ ID NO: 2318 and an AAVPHP.N capsid. In some embodiments, the AAV particle comprises a viral genome comprising SEQ ID NO: 2318 and an AAV2 variant capsid. In some embodiments, the AAV particle comprises a viral genome comprising SEQ ID NO: 2318 and an AAV2/3 variant capsid. In some embodiments, the AAV particle comprises a viral genome comprising SEQ ID NO: 2318 and a capsid comprising SEQ ID NO: 1. In some embodiments, the AAV particle comprises a viral genome comprising SEQ ID NO: 2318 and a capsid encoded by a nucleic acid sequence comprising SEQ ID NO: 2. In some embodiments, the AAV particle comprises a viral genome comprising SEQ ID NO: 2318 and a capsid comprising SEQ ID NO: 4534. In some embodiments, the AAV particle comprises a viral genome comprising SEQ ID NO: 2318 and a capsid encoded by a nucleic acid sequence comprising SEQ ID NO: 3. In some embodiments, the AAV particle comprises a viral genome comprising SEQ ID NO: 2318 and a capsid comprising SEQ ID NO: 138. In some embodiments, the AAV particle comprises a viral genome comprising SEQ ID NO: 2318 and a capsid encoded by a nucleic acid sequence comprising SEQ ID NO: 137. In some embodiments, the AAV particle comprises a viral genome comprising SEQ ID NO: 2318 and a capsid comprising SEQ ID NO: 5. In some embodiments, the AAV particle comprises a viral genome comprising SEQ ID NO: 2318 and a capsid encoded by a nucleic acid sequence comprising SEQ ID NO: 6. In some embodiments, the AAV particle comprises a viral genome comprising SEQ ID NO: 2318 and a capsid comprising SEQ ID NO: 4. In some embodiments, the AAV particle comprises a viral genome comprising SEQ ID NO: 2318 and a capsid comprising SEQ ID NO: 11. In some embodiments, the AAV particle comprises a viral genome comprising SEQ ID NO: 2318 and a capsid comprising SEQ ID NO: 2679. In some embodiments, the AAV particle comprises a viral genome comprising SEQ ID NO: 2318 and a capsid comprising SEQ ID NO: 2809. In some embodiments, the AAV particle comprises a viral genome comprising SEQ ID NO: 2318 and a capsid comprising SEQ ID NO: 2871.

In some embodiments, the AAV particle comprises a viral genome comprising SEQ ID NO: 2319 and a VOY101 capsid. In some embodiments, the AAV particle comprises a viral genome comprising SEQ ID NO: 2319 and a VOY201 capsid. In some embodiments, the AAV particle comprises a viral genome comprising SEQ ID NO: 2319 and an AAV9 capsid. In some embodiments, the AAV particle comprises a viral genome comprising SEQ ID NO: 2319 and an AAV9K449R capsid. In some embodiments, the AAV particle comprises a viral genome comprising SEQ ID NO: 2319 and an AAVPHP.B capsid. In some embodiments, the AAV particle comprises a viral genome comprising SEQ ID NO: 2319 and an AAVPHP.N capsid. In some embodiments, the AAV particle comprises a viral genome comprising SEQ ID NO: 2319 and an AAV2 variant capsid. In some embodiments, the AAV particle comprises a viral genome comprising SEQ ID NO: 2319 and an AAV2/3 variant capsid. In some embodiments, the AAV particle comprises a viral genome comprising SEQ ID NO: 2319 and a capsid comprising SEQ ID NO: 1. In some embodiments, the AAV particle comprises a viral genome comprising SEQ ID NO: 2319 and a capsid encoded by a nucleic acid sequence comprising SEQ ID NO: 2. In some embodiments, the AAV particle comprises a viral genome comprising SEQ ID NO: 2319 and a capsid comprising SEQ ID NO: 4534. In some embodiments, the AAV particle comprises a viral genome comprising SEQ ID NO: 2319 and a capsid encoded by a nucleic acid sequence comprising SEQ ID NO: 3. In some embodiments, the AAV particle comprises a viral genome comprising SEQ ID NO: 2319 and a capsid comprising SEQ ID NO: 138. In some embodiments, the AAV particle comprises a viral genome comprising SEQ ID NO: 2319 and a capsid encoded by a nucleic acid sequence comprising SEQ ID NO: 137. In some embodiments, the AAV particle comprises a viral genome comprising SEQ ID NO: 2319 and a capsid comprising SEQ ID NO: 5. In some embodiments, the AAV particle comprises a viral genome comprising SEQ ID NO: 2319 and a capsid encoded by a nucleic acid sequence comprising SEQ ID NO: 6. In some embodiments, the AAV particle comprises a viral genome comprising SEQ ID NO: 2319 and a capsid comprising SEQ ID NO: 4. In some embodiments, the AAV particle comprises a viral genome comprising SEQ ID NO: 2319 and a capsid comprising SEQ ID NO: 11. In some embodiments, the AAV particle comprises a viral genome comprising SEQ ID NO: 2319 and a capsid comprising SEQ ID NO: 2679. In some embodiments, the AAV particle comprises a viral genome comprising SEQ ID NO: 2319 and a capsid comprising SEQ ID NO: 2809. In some embodiments, the AAV particle comprises a viral genome comprising SEQ ID NO: 2319 and a capsid comprising SEQ ID NO: 2871.

In some embodiments, the AAV particle comprises a viral genome comprising SEQ ID NO: 2320 and a VOY101 capsid. In some embodiments, the AAV particle comprises a viral genome comprising SEQ ID NO: 2320 and a VOY201 capsid. In some embodiments, the AAV particle comprises a viral genome comprising SEQ ID NO: 2320 and an AAV9 capsid. In some embodiments, the AAV particle comprises a viral genome comprising SEQ ID NO: 2320 and an AAV9K449R capsid. In some embodiments, the AAV particle comprises a viral genome comprising SEQ ID NO: 2320 and an AAVPHP.B capsid. In some embodiments, the AAV particle comprises a viral genome comprising SEQ ID NO: 2320 and an AAVPHP.N capsid. In some embodiments, the AAV particle comprises a viral genome comprising SEQ ID NO: 2320 and an AAV2 variant capsid. In some embodiments, the AAV particle comprises a viral genome comprising SEQ ID NO: 2320 and an AAV2/3 variant capsid. In some embodiments, the AAV particle comprises a viral genome comprising SEQ ID NO: 2320 and a capsid comprising SEQ ID NO: 1. In some embodiments, the AAV particle comprises a viral genome comprising SEQ ID NO: 2320 and a capsid encoded by a nucleic acid sequence comprising SEQ ID NO: 2. In some embodiments, the AAV particle comprises a viral genome comprising SEQ ID NO: 2320 and a capsid comprising SEQ ID NO: 4534. In some embodiments, the AAV particle comprises a viral genome comprising SEQ ID NO: 2320 and a capsid encoded by a nucleic acid sequence comprising SEQ ID NO: 3. In some embodiments, the AAV particle comprises a viral genome comprising SEQ ID NO: 2320 and a capsid comprising SEQ ID NO: 138. In some embodiments, the AAV particle comprises a viral genome comprising SEQ ID NO: 2320 and a capsid encoded by a nucleic acid sequence comprising SEQ ID NO: 137. In some embodiments, the AAV particle comprises a viral genome comprising SEQ ID NO: 2320 and a capsid comprising SEQ ID NO: 5. In some embodiments, the AAV particle comprises a viral genome comprising SEQ ID NO: 2320 and a capsid encoded by a nucleic acid sequence comprising SEQ ID NO: 6. In some embodiments, the AAV particle comprises a viral genome comprising SEQ ID NO: 2320 and a capsid comprising SEQ ID NO: 4. In some embodiments, the AAV particle comprises a viral genome comprising SEQ ID NO: 2320 and a capsid comprising SEQ ID NO: 11. In some embodiments, the AAV particle comprises a viral genome comprising SEQ ID NO: 2320 and a capsid comprising SEQ ID NO: 2679. In some embodiments, the AAV particle comprises a viral genome comprising SEQ ID NO: 2320 and a capsid comprising SEQ ID NO: 2809. In some embodiments, the AAV particle comprises a viral genome comprising SEQ ID NO: 2320 and a capsid comprising SEQ ID NO: 2871.

In some embodiments, the AAV particle comprises a viral genome comprising SEQ ID NO: 2321 and a VOY101 capsid. In some embodiments, the AAV particle comprises a viral genome comprising SEQ ID NO: 2321 and a VOY201 capsid. In some embodiments, the AAV particle comprises a viral genome comprising SEQ ID NO: 2321 and an AAV9 capsid. In some embodiments, the AAV particle comprises a viral genome comprising SEQ ID NO: 2321 and an AAV9K449R capsid. In some embodiments, the AAV particle comprises a viral genome comprising SEQ ID NO: 2321 and an AAVPHP.B capsid. In some embodiments, the AAV particle comprises a viral genome comprising SEQ ID NO: 2321 and an AAVPHP.N capsid. In some embodiments, the AAV particle comprises a viral genome comprising SEQ ID NO: 2321 and an AAV2 variant capsid. In some embodiments, the AAV particle comprises a viral genome comprising SEQ ID NO: 2321 and an AAV2/3 variant capsid. In some embodiments, the AAV particle comprises a viral genome comprising SEQ ID NO: 2321 and a capsid comprising SEQ ID NO: 1. In some embodiments, the AAV particle comprises a viral genome comprising SEQ ID NO: 2321 and a capsid encoded by a nucleic acid sequence comprising SEQ ID NO: 2. In some embodiments, the AAV particle comprises a viral genome comprising SEQ ID NO: 2321 and a capsid comprising SEQ ID NO: 4534. In some embodiments, the AAV particle comprises a viral genome comprising SEQ ID NO: 2321 and a capsid encoded by a nucleic acid sequence comprising SEQ ID NO: 3. In some embodiments, the AAV particle comprises a viral genome comprising SEQ ID NO: 2321 and a capsid comprising SEQ ID NO: 138. In some embodiments, the AAV particle comprises a viral genome comprising SEQ ID NO: 2321 and a capsid encoded by a nucleic acid sequence comprising SEQ ID NO: 137. In some embodiments, the AAV particle comprises a viral genome comprising SEQ ID NO: 2321 and a capsid comprising SEQ ID NO: 5. In some embodiments, the AAV particle comprises a viral genome comprising SEQ ID NO: 2321 and a capsid encoded by a nucleic acid sequence comprising SEQ ID NO: 6. In some embodiments, the AAV particle comprises a viral genome comprising SEQ ID NO: 2321 and a capsid comprising SEQ ID NO: 4. In some embodiments, the AAV particle comprises a viral genome comprising SEQ ID NO: 2321 and a capsid comprising SEQ ID NO: 11. In some embodiments, the AAV particle comprises a viral genome comprising SEQ ID NO: 2321 and a capsid comprising SEQ ID NO: 2679. In some embodiments, the AAV particle comprises a viral genome comprising SEQ ID NO: 2321 and a capsid comprising SEQ ID NO: 2809. In some embodiments, the AAV particle comprises a viral genome comprising SEQ ID NO: 2321 and a capsid comprising SEQ ID NO: 2871.

### II. Formulation and Delivery

### Pharmaceutical Compositions

According to the present disclosure the AAV particles may be prepared as pharmaceutical compositions. It will be understood that such compositions necessarily comprise one or more active ingredients and, most often, a pharmaceutically acceptable excipient.

Relative amounts of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the AAV particle pharmaceutical compositions described herein may comprise at least one payload. As a non-limiting example, the pharmaceutical compositions may contain an AAV particle with 1, 2, 3, 4 or 5 payloads. In some embodiments, the pharmaceutical composition may contain a nucleic acid encoding a payload construct encoding proteins selected from antibodies and/or antibody-based compositions.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients, or subjects.

### Formulations

The AAV particles can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload.

Formulations of the present disclosure can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an AAV particle carrying a payload region encoding the polypeptides or to the antibody or antibody-based composition encoded by a viral genome of by an AAV particle as described herein.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, the AAV particles may be formulated in phosphate buffered saline (PBS), in combination with an ethylene oxide/propylene oxide copolymer (also known as Pluronic or poloxamer).

In some embodiments, the AAV particles may be formulated in PBS with 0.001% Pluronic acid (F-68) (poloxamer 188) at a pH of about 7.0.

In some embodiments, the AAV particles may be formulated in PBS with 0.001% Pluronic acid (F-68) (poloxamer 188) at a pH of about 7.3.

In some embodiments, the AAV particles may be formulated in PBS with 0.001% Pluronic acid (F-68) (poloxamer 188) at a pH of about 7.4.

In some embodiments, the AAV particles may be formulated in a solution comprising sodium chloride, sodium phosphate and an ethylene oxide/propylene oxide copolymer.

In some embodiments, the AAV particles may be formulated in a solution comprising sodium chloride, sodium phosphate dibasic, sodium phosphate monobasic and poloxamer 188/Pluronic acid (F-68).

In some embodiments, the AAV particles may be formulated in a solution comprising about 180 mM sodium chloride, about 10 mM sodium phosphate and about 0.001% poloxamer 188. In some embodiments, this formulation may be at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate in the final solution may be 1 mM-50 mM. As non-limiting examples, the concentration of sodium phosphate in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the invention may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

Relative amounts of the active ingredient (e.g. AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

In some embodiments, the AAV formulations described herein may contain sufficient AAV particles for expression of at least one expressed functional antibody or antibody-based composition. As a non-limiting example, the AAV particles may contain viral genomes encoding 1, 2, 3, 4, or 5 functional antibodies.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, AAV particle formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

Pharmaceutical composition formulations of AAV particles disclosed herein may include cations or anions. In some embodiments, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

### III. Administration and Dosing

### Administration

The AAV particles of the present disclosure may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions may be administered in away which allows them to cross the blood-brain barrier, vascular barrier, or other epithelial barrier. The AAV particles of the present disclosure may be administered in any suitable form, either as a liquid solution or suspension, as a solid form suitable for liquid solution or suspension in a liquid solution. The AAV particles may be formulated with any appropriate and pharmaceutically acceptable excipient.

In some embodiments, the AAV particles of the present disclosure may be delivered to a subject via a single route administration.

In some embodiments, the AAV particles of the present disclosure may be delivered to a subject via a multi-site route of administration. A subject may be administered at 2, 3, 4, 5, or more than 5 sites.

In some embodiments, a subject may be administered the AAV particles of the present disclosure using a bolus infusion.

In some embodiments, a subject may be administered the AAV particles of the present disclosure using sustained delivery over a period of minutes, hours, or days. The infusion rate may be changed depending on the subject, distribution, formulation or another delivery parameter.

In some embodiments, the AAV particles may be delivered by more than one route of administration. As non-limiting examples of combination administrations, AAV particles may be delivered by intrathecal and intracerebroventricular, or by intravenous and intraparenchymal administration.

### Intravenous Administration

In some embodiments, the AAV particles may be administered to a subject by systemic administration.

In some embodiments, the systemic administration is intravenous administration.

In another embodiment, the systemic administration is intraarterial administration.

In some embodiments, the AAV particles of the present disclosure may be administered to a subject by intravenous administration.

In some embodiments, the intravenous administration may be achieved by subcutaneous delivery.

In some embodiments, the intravenous administration may be achieved by a tail vein injection (e.g., in a mouse model).

In some embodiments, the intravenous administration may be achieved by retro-orbital injection.

### Administration to the CNS

In some embodiments, the AAV particles may be delivered by direct injection into the brain. As a non-limiting example, the brain delivery may be by intrahippocampal administration.

In some embodiments, the AAV particles of the present disclosure may be administered to a subject by intraparenchymal administration. In some embodiments, the intraparenchymal administration is to tissue of the central nervous system.

In some embodiments, the AAV particles of the present disclosure may be administered to a subject by intracranial delivery (See. e.g., U.S. Pat. No. 8,119,611; the content of which is incorporated herein by reference in its entirety).

In some embodiments, the AAV particles may be delivered by injection into the CSF pathway. Non-limiting examples of delivery to the CSF pathway include intrathecal and intracerebroventricular administration.

In some embodiments, the AAV particles may be delivered to the brain by systemic delivery. As a non-limiting example, the systemic delivery may be by intravascular administration. As a non-limiting example, the systemic or intravascular administration may be intravenous.

In some embodiments, the AAV particles of the present disclosure may be delivered by intraocular delivery route. A non-limiting example of intraocular administration include an intravitreal injection.

### Intramuscular Administration

In some embodiments, the AAV particles may be delivered by intramuscular administration. Whilst not wishing to be bound by theory, the multi-nucleated nature of muscle cells provides an advantage to gene transduction subsequent to AAV delivery. Cells of the muscle are capable of expressing recombinant proteins with the appropriate post-translational modifications. The enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered.

In some embodiments, the AAV particles of the present disclosure may be delivered by intramuscular delivery route. (See, e.g., U.S. Pat. No. 6,506,379: the content of which is incorporated herein by reference in its entirety). Non-limiting examples of intramuscular administration include an intravenous injection or a subcutaneous injection.

In some embodiments, the AAV particles of the present disclosure are administered to a subject and transduce muscle of a subject. As a non-limiting example, the AAV particles are administered by intramuscular administration.

In some embodiments, the AAV particles of the present disclosure may be administered to a subject by subcutaneous administration.

In some embodiments, the intramuscular administration is via systemic delivery.

In some embodiments, the intramuscular administration is via intravenous delivery.

In some embodiments, the intramuscular administration is via direct injection to the muscle.

In some embodiments, the muscle is transduced by administration, and this is referred to as intramuscular administration.

In some embodiments, the intramuscular delivery comprises administration at one site.

In some embodiments, the intramuscular delivery comprises administration at more than one site. In some embodiments, the intramuscular delivery comprises administration at two sites. In some embodiments, the intramuscular delivery comprises administration at three sites. In some embodiments, the intramuscular delivery comprises administration at four sites. In some embodiments, the intramuscular delivery comprises administration at more than four sites.

In some embodiments, intramuscular delivery is combined with at least one other method of administration.

In some embodiments, the AAV particles that may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U.S. Patent Publication Nos. US20100240739 and US20100130594; the content of each of which is incorporated herein by reference in their entirety).

In some embodiments, the AAV particles of the present disclosure may be administered to a subject by intraparenchymal administration. In some embodiments, the intraparenchymal administration is to muscle tissue.

In some embodiments, the AAV particles of the present disclosure are delivered as described in Bright et al 2015 (Neurobiol Aging. 36(2):693-709), the contents of which are herein incorporated by reference in their entirety.

In some embodiments, the AAV particles of the present disclosure are administered to the gastrocnemius muscle of a subject.

In some embodiments, the AAV particles of the present disclosure are administered to the bicep femorii of the subject.

In some embodiments, the AAV particles of the present disclosure are administered to the tibialis anterior muscles.

In some embodiments, the AAV particles of the present disclosure are administered to the soleus muscle.

Depot Administration

As described herein, in some embodiments, pharmaceutical compositions, AAV particles of the present disclosure are formulated in depots for extended release. Generally, specific organs or tissues (“target tissues”) are targeted for administration.

In some aspects, pharmaceutical compositions, AAV particles of the present disclosure are spatially retained within or proximal to target tissues. Provided are methods of providing pharmaceutical compositions, AAV particles, to target tissues of mammalian subjects by contacting target tissues (which comprise one or more target cells) with pharmaceutical compositions, AAV particles, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, AAV particles, that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of pharmaceutical compositions, AAV particles, administered to subjects are present intracellularly at a period of time following administration. For example, intramuscular injection to mammalian subjects may be performed using aqueous compositions comprising pharmaceutical compositions, AAV particles of the present disclosure and one or more transfection reagents, and retention is determined by measuring the amount of pharmaceutical compositions, AAV particles, present in muscle cells.

Certain aspects are directed to methods of providing pharmaceutical compositions, AAV particles of the present disclosure to a target tissues of mammalian subjects, by contacting target tissues (comprising one or more target cells) with pharmaceutical compositions, AAV particles under conditions such that they are substantially retained in such target tissues. Pharmaceutical compositions, AAV particles comprise enough active ingredient such that the effect of interest is produced in at least one target cell. In some embodiments, pharmaceutical compositions, AAV particles generally comprise one or more cell penetration agents, although “naked” formulations (such as without cell penetration agents or other agents) are also contemplated, with or without pharmaceutically acceptable carriers.

### Delivery

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particle or pharmaceutical compositions of the present disclosure may be administered or delivered using the methods for delivering proteins using AAV vectors described in European Patent Application No. EP2678433, the contents of which are herein incorporated by reference in their entirety.

In some embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

### Delivery to Cells

The present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

The present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

### Dose and Regimen

The present disclosure provides methods of administering AAV particles in accordance with the disclosure to a subject in need thereof. The pharmaceutical, diagnostic, or prophylactic AAV particles and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific AAV particle employed; the duration of the treatment; drugs used in combination or coincidental with the specific AAV particle employed; and like factors well known in the medical arts.

In certain embodiments, AAV particle pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations may be converted to vg or viral genomes per kg or into total viral genomes administered by one of skill in the art.

In certain embodiments, AAV particle pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, AAV particles may be administered at 50 μl/site and/or 150 μl/site.

In certain embodiments, the desired dosage may be delivered using multiple administrations (e.g., two, three, four, or more than four administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the AAV particles of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

In some embodiments, delivery of the AAV particles of the present disclosure to a subject provides neutralizing activity to a subject. The neutralizing activity can be for at least 1 month, 2 months, 3 months, 4 months, 5 months, 6 months, 7 months, 8 months, 9 months, 10 months, 10 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, delivery of the AAV particles of the present disclosure results in minimal serious adverse events (SAEs) as a result of the delivery of the AAV particles.

In some embodiments, delivery of AAV particles may comprise a total dose between about 1×10⁶VG and about 1×10¹⁶VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³VG. As another non-limiting example, the total dose is 2.1×10¹² VG.

In some embodiments, delivery of AAV particles may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10, 3×10¹⁵, 4×10¹⁵, 5×10¹⁵, 6×10¹⁵, 7×10¹⁵, 8×10¹⁵, 9×10¹⁵, or 1×10¹⁶ VG/mL. In some embodiments, the delivery comprises a composition concentration of 1×10¹³ VG/mL. In some embodiments, the delivery comprises a composition concentration of 2.1×10¹² VG/mL.

### Combinations

The AAV particles may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### Measurement of Expression

Expression of payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, immunohistochemistry, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

In some embodiments, the ELISA assays used are those described in Liu et al 2016, the contents of which are herein incorporated by reference in their entirety (Liu, W et al., 2016 J Neurosci 36(49):12425-12435).

### IV. Methods and Uses of the Compositions

The present disclosure provides a method for treating a disease, disorder and/or condition in a mammalian subject, including a human subject, comprising administering to the subject any of the AAV particles described herein or administering to the subject any of the described compositions, including pharmaceutical compositions, described herein.

In some embodiments, the AAV particles of the present disclosure are administered to a subject prophylactically.

In some embodiments, the AAV particles of the present disclosure are administered to a subject having at least one of the diseases described herein.

In some embodiments, the AAV particles of the present disclosure are administered to a subject to treat a disease or disorder described herein. The subject may have the disease or disorder or may be at-risk to developing the disease or disorder.

In some embodiments, the AAV particles of the present disclosure are part of an active immunization strategy to protect against diseases and disorders. In an active immunization strategy, a vaccine or AAV particles are administered to a subject to prevent an infectious disease by activating the subject's production of antibodies that can fight off invading bacteria or viruses.

In some embodiments, the AAV particles of the present disclosure are part of a passive immunization strategy. In a passive immunization strategy, antibodies against a particular infectious agent are given directly to the subject.

In some embodiments, the AAV particles of the present disclosure may be used for passive immunotherapy of tauopathy, (e.g. Alzheimer Disease or Frontotemporal Dementia), as described in Liu et al, the contents of which are herein incorporated by reference in their entirety (Liu, W et al., 2016 J Neurosci 36(49):12425-12435). As a non-limiting example, the AAV particles of the present disclosure may encode a PHF1 antibody. Heavy and light chains of the PHF1 antibody may be linked by a Tav2A and/or Furin 2A linker sequence. Antibody expression may be under the control of a CAG promoter. The AAV particle may comprise, as a non-limiting example, an AAVrh.10 serotype capsid. Further, these PHF1 encoding AAV particles may be administered by bilateral intraparenchymal delivery directly to the hippocampus. Such treatment with AAV-PHF1 may result in a 50-fold increase in antibody levels in the hippocampus as compared to antibody levels subsequent to systemic administration. Neuropathological tau species in the hippocampus may be reduced as much as 80-90% and hippocampal atrophy may be fully rescued after treatment with AAV particles of the present disclosure.

In some embodiments, the AAV particles of the present disclosure may be used to treat tauopathy as described in Ising et al, the contents of which are herein incorporated by reference in their entirety (Ising, C et al., 2017 J Exp Med April 17, Epub ahead of print). As a non-limiting example, the AAV particles of the present disclosure may encode an HJ8.5, HJ8.7, or Tau5 antibody or a single chain variable fragment (scFv) derived therefrom. Heavy and light chains of the HJ8.5 antibody or scFv may be linked by variable length linker sequences and may be flexible glycine and/or serine linkers. The AAV particle may comprise, as a non-limiting example, an AAV2/8 serotype. Further, these HJ8.5, HJ8.7 or Tau5 encoding AAV particles may be administered by bilateral intracerebroventricular delivery. Such treatment with HJ8.5, HJ8.7 or Tau5 encoding AAV particles may result in a significant reduction in neuropathological tau species in the hippocampus.

Passive immunization by intravenous (or intraperitoneal in mice) delivery of antibody has been shown to result in substantial serum levels of antibody (Chai et al., 2011, J Biol Chem., 286, 34457-34467, the contents of which are herein incorporated by reference in their entirety) and reduced tau pathology in mouse models of tauopathy (e.g., P301L or P301S mice). However, these reductions in tau pathology are considered modest (e.g., about 34%) and require high and repeated dosing to achieve. Passive immunization strategies are thought to be limited in their ability to deliver large quantity of antibody to the brain, which may limit efficacy, and are also challenged in addressing intracellular aggregates. In some embodiments, delivery of an AAV particle comprising a viral genome encoding an anti-tau antibody can be used to overcome the limitations seen with passive immunization strategies.

In some embodiments, the administration of AAV particles of the present disclosure may result in substantially higher antibody levels in the target tissue (e.g., CNS) of the subject than if anti-tau antibodies were administered by passive immunization. In some embodiments, AAV mediated delivery can result in 1.5-16 fold higher antibody levels in the brain than if delivered by passive immunization. Whilst not wishing to be bound by theory, passive immunization is thought to generate 20-40 ng of antibody per mg of protein in the brain of the subject. In some embodiments, AAV-mediated delivery results in antibody levels 2-5× above the levels seen with passive immunization. In some embodiments, AAV-mediated delivery results in antibody levels 1.5-3× above the levels seen with passive immunization. In some embodiments, AAV-mediated delivery results in antibody levels 5-10× above the levels seen with passive immunization. In some embodiments, AAV-mediated delivery results in antibody levels 8-16× above the levels seen with passive immunization.

### Diagnostic Applications

The AAV particles of the present disclosure may be used for diagnostic purposes or as diagnostic tools for any disease or disorder. As non-limiting examples, the AAV particles of the present disclosure or the antibodies encoded within the viral genome therein may be used as a biomarker for disease diagnosis. As a second non-limiting example, the AAV particles of the present disclosure or the antibodies encoded within the viral genome therein may be used for diagnostic imaging purposes, e.g., MRI, PET, CT or ultrasound.

### Preventative Applications

The AAV particles of the present disclosure or the antibodies encoded by the viral genome therein may be used to prevent disease or stabilize the progression of disease. In some embodiments, the AAV particles of the present disclosure are used to as a prophylactic to prevent a disease or disorder in the future. In some embodiments, the AAV particles of the present disclosure are used to halt further progression of a disease or disorder. As a non-limiting example, the AAV particles may be used in a manner similar to that of a vaccine.

### Research Applications

The AAV particles of the present disclosure or the antibodies encoded by the viral genome therein may also be used as research tools. The AAV particles may be used as in any research experiment, e.g., in vivo or in vitro experiments. In a non-limiting example, the AAV particles may be used in cultured cells. The cultured cells may be derived from any origin known to one with skill in the art, and may be as non-limiting examples, derived from a stable cell line, an animal model or a human patient or control subject. In a non-limiting example, the AAV particles may be used in in vivo experiments in animal models (i.e., mouse, rat, rabbit, dog, cat, non-human primate, guinea pig, ferret, c-elegans, drosophila, zebrafish, or any other animal used for research purposes, known in the art). In another non-limiting example, the AAV particles may be used in human research experiments or human clinical trials.

### Combination Applications

The AAV particles may be used as a combination therapy with any other therapeutic molecule known in the art. The therapeutic molecule may be approved by the US Food and Drug Administration or may be in clinical trial or at the preclinical research stage. The therapeutic molecule may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Therapeutic Applications

The present disclosure additionally provides a method for treating neurological diseases and/or disorders in a mammalian subject, including a human subject, comprising administering to the subject any of the AAV particles. In some cases, neurological diseases and/or disorders treated according to methods described herein include indications involving irregular expression or aggregation of tau. Such indications may include, but are not limited to Alzheimer's disease (AD), frontotemporal dementia and parkinsonism linked to chromosome 17 (FTDP-17), Frontotemporal lobar degeneration (FTLD), Frontotemporal dementia (FTD), chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis.

In some embodiments, methods of treating neurological diseases and/or disorders in a subject in need thereof may comprise the steps of: (1) deriving, generating and/or selecting an anti-tau antibody, antibody-based composition or fragment thereof: (2) producing an AAV particle with a viral genome that includes a payload region encoding the selected antibody of (1); and (3) administering the AAV particle (or pharmaceutical composition thereof) to the subject.

The present disclosure provides a method for administering to a subject in need thereof, including a human subject, a therapeutically effective amount of the AAV particles to slow, stop or reverse disease progression. As a non-limiting example, disease progression may be measured by cognitive tests such as, but not limited to, the Mini-Mental State Exam (MMSE) or other similar diagnostic tool(s), known to those skilled in the art. As another non-limiting example, disease progression may be measured by change in the pathological features of the brain. CSF or other tissues of the subject, such as, but not limited to a decrease in levels of tau (either soluble or insoluble). In some embodiments levels of insoluble hyperphosphorylated tau are decreased. In some embodiments levels of soluble tau are decreased. In some embodiments both soluble and insoluble tau are decreased. In some embodiments, levels of insoluble hyperphosphorylated tau are increased. In some embodiments levels of soluble tau are increased. In some embodiments both insoluble and soluble tau levels are increased. In some embodiments, neurofibrillary tangles are decreased in size, number, density, or combination thereof. In another embodiment, neurofibrillary tangles are increased in size, number, density or combination thereof.

### Alzheimer's Disease

Alzheimer Disease (AD) is a debilitating neurodegenerative disease currently afflicting more than 35 million people worldwide, with that number expected to double in coming decades. Symptomatic treatments have been available for many years but these treatments do not address the underlying pathophysiology. Recent clinical trials using these and other treatments have largely failed and, to date, no known cure has been identified.

The AD brain is characterized by the presence of two forms of pathological aggregates, the extracellular plaques composed of β-amyloid (Aβ) and the intracellular neurofibrillary tangles (NFT) comprised of hyperphosphorylated microtubule associated protein tau. Based on early genetic findings, β-amyloid alterations were thought to initiate disease, with changes in tau considered downstream. Thus, most clinical trials have been Aβ-centric. Although no mutations of the tau gene have been linked to AD, such alterations have been shown to result in a family of dementias known as tauopathies, demonstrating that changes in tau can contribute to neurodegenerative processes. Tau is normally a very soluble protein known to associate with microtubules based on the extent of its phosphorylation. Hyperphosphorylation of tau depresses its binding to microtubules and microtubule assembly activity. In tauopathies, the tau becomes hyperphosphorylated, misfolds and aggregates as NFT of paired helical filaments (PHF), twisted ribbons or straight filaments. In AD, NFT pathology, rather than plaque pathology, correlates more closely with neuropathological markers such as neuronal loss, synaptic deficits, severity of disease and cognitive decline. NFT pathology marches through the brain in a stereotyped manner and animal studies suggest a trans-cellular propagation mechanism along neuronal connections.

Several approaches have been proposed for therapeutically interfering with progression of tau pathology and preventing the subsequent molecular and cellular consequences. Given that NFT are composed of a hyperphosphorylated, misfolded and aggregated form of tau, interference at each of these stages has yielded the most avidly pursued set of targets. Introducing agents that limit phosphorylation, block misfolding or prevent aggregation have all generated promising results. Passive and active immunization with late stage anti-phospho-tau antibodies in mouse models have led to dramatic decreases in tau aggregation and improvements in cognitive parameters. It has also been suggested that introduction of anti-tau antibodies can prevent the trans-neuronal spread of tau pathology.

The vectored antibody delivery (VAD) of tau disease associated antibodies of the present disclosure may be used to treat subjects suffering from AD and other tauopathies. In some cases, methods of the present disclosure may be used to treat subjects suspected of developing AD or other tauopathies.

### Frontotemporal Dementia and Parkinsonism Linked to Chromosome 17 (FTDP-17)

Although Alzheimer's disease is, in part, characterized by the presence of tau pathology, no known mutations in the tau gene have been causally linked to the disease. Mutations in the tau gene have been shown to lead to an autosomal dominantly inherited tauopathy known as frontotemporal dementia and parkinsonism linked to chromosome 17 (FTDP-17) and demonstrate that alterations in tau can lead to neurodegenerative changes in the brain. Mutations in the tau gene that lead to FTDP-17 are thought to influence splicing patterns, thereby leading to an elevated proportion of tau with four microtubule binding domains (rather than three). These molecules are considered to be more amyloidogenic, meaning they are more likely to become hyperphosphorylated and more likely to aggregate into NFT (Hutton, M. et al., 1998, Nature 393(6686):702-5). Although physically and behaviorally, FTDP-17 patients can appear quite similar to Alzheimer's disease patients, at autopsy FTDP-17 brains lack the prominent Aβ plaque pathology of an AD brain (Gotz, J. et al., 2012, British Journal of Pharmacology 165(5):1246-59). Therapeutically targeting the aggregates of tau protein may ameliorate and prevent degenerative changes in the brain and potentially lead to improved cognitive ability.

As of today, there is no treatment to prevent, slow the progression, or cure FTDP-17. Medication may be prescribed to reduce aggressive, agitated or dangerous behavior. There remains a need for therapy affecting the underlying pathophysiology, such as antibody therapies targeting tau protein.

In some embodiments, the vectored antibody delivery of the present disclosure may be used to treat subjects suffering from FTDP-17. In some cases, methods of the present disclosure may be used to treat subjects suspected of developing FTDP-17.

### Chronic Traumatic Encephalopathy

Unlike the genetically linked tauopathies, chronic traumatic encephalopathy is a degenerative tauopathy linked to repeated head injuries. The disease was first described in boxers who behaved “punch drunk” and has since been identified primarily in athletes that play American football, ice hockey, wrestling and other contact sports. The brains of those suffering from CTE are characterized by distinctive patterns of brain atrophy accompanied by accumulation of hyperphosphorylated species of aggregated tau in NFT. In CTE, pathological changes in tau are accompanied by a number of other pathobiological processes, such as inflammation (Daneshvar, D. H. et al., 2015 Mol Cell Neurosci 66(Pt B): 81-90). Targeting the tau aggregates may provide reprieve from the progression of the disease and may allow cognitive improvement.

As of today, there is no medical therapy to treat or cure CTE. The condition is only diagnosed after death, due to lack of in vivo techniques to identify CTE specific biomarkers. There remains a need for therapy affecting the underlying pathophysiology, such as antibody therapies targeting tau protein.

In some embodiments, the vectored antibody delivery methods of the present disclosure may be used to treat subjects suffering from CTE. In some cases, methods of the present disclosure may be used to treat subjects suspected of developing CTE.

### Prion Diseases

Prion diseases, also known as transmissible spongiform encephalopathies (TSEs), are a group of rare progressive conditions affecting the nervous system. The related conditions are rare and are typically caused by mutations in the PRNP gene which enables production of the prion protein. Gene mutations lead to an abnormally structured prion protein. Alternatively, the abnormal prion may be acquired by exposure from an outside source, e.g. by consumption of beef products containing the abnormal prion protein. Abnormal prions are misfolded, causing the brain tissue to degenerate rapidly. Prion diseases include, but are not limited to, Creutzfeldt-Jakob disease (CJD), Gerstmann-Sträussler-Scheinker syndrome (GSS), fatal insomnia (FFI), variably protease-sensitive prionopathy (VPSPr), and kuru. Prion diseases are rare. Approximately 350 cases of prion diseases are diagnosed in the US annually.

CJD is a degenerative brain disorder characterized by problems with muscular coordination, personality changes including mental impairment, impaired vision, involuntary muscle jerks, weakness and eventually coma. The most common categories of CJD are sporadic, hereditary due to a genetic mutation, and acquired. Sporadic CJD is the most common form affecting people with no known risk factors for the disease. The acquired form of CJD is transmitted by exposure of the brain and nervous system tissue to the prion. As an example, variant CJD (vCDJ) is linked to a bovine spongiform encephalopathy (BSE), also known as a ‘mad cow’ disease. CJD is fatal and patients typically die within one year of diagnosis.

Prion diseases are associated with an infectious agent consisting of an alternative conformational isoform of the prion protein, PrPSc. PrPSc replication is considered to occur through an induction of the infectious prion in the normal prion protein (PrPC). The replication occurs without a nucleic acid.

As of today, there is no therapy to manage or cure CJD, or other prion diseases. Typically, treatment is aimed at alleviating symptoms and increasing comfortability of the patient, e.g. with pain relievers. There remains a need for therapy affecting the underlying pathophysiology, such as antibody therapies targeting the prion protein.

In some embodiments, vectored antibody delivery methods of the present disclosure may be used to treat subjects suffering from a prion disease. In some cases, methods of the present disclosure may be used to treat subjects suspected of developing a prion disease.

### Neurodegeneration and Stroke

Neurodegenerative diseases and other diseases of the nervous system share many common features. Neurodegenerative diseases, in particular, are a group of conditions characterized by progressive loss of neuronal structure and function, ultimately leading to neuronal cell death. Neurons are the building blocks of the nervous system(s) and are generally not able to reproduce and/or be replaced, and therefore neuron damage and/or death is especially devastating. Other, non-degenerating diseases that lead to neuronal cell loss, such as stroke, have similarly debilitating outcomes. Targeting molecules that contribute to the deteriorating cell structure or function may prove beneficial generally for treatment of nervous system diseases, neurodegenerative disease and/or stroke.

Certain molecules are believed to have inhibitory effects on neurite outgrowth, contributing to the limited ability of the central nervous system to repair. Such molecules include, but are not limited to, myelin associated proteins, such as, but not limited to, RGM (Repulsive guidance molecule), NOGO (Neurite outgrowth inhibitor), NOGO receptor, MAG (myelin associated glycoprotein), and MAI (myelin associated inhibitor). In some embodiments, the vectored antibody delivery of the present disclosure is utilized to target the aforementioned antigens (e.g., neurite outgrowth inhibitors).

Many neurodegenerative diseases are associated with aggregation of misfolded proteins, including, but not limited to, alpha synuclein, tau, amyloid β, prion proteins, TDP-43, and huntingtin (see, e.g. De Genst et al., 2014, Biochim Biophys Acta; 1844(11):1907-1919, and Yu et al., 2013, Neurotherapeutics.; 10(3): 459-472, references therein). The aggregation results from disease-specific conversion of soluble proteins to an insoluble, highly ordered fibrillary deposit. This conversion is thought to prevent the proper disposal or degradation of the misfolded protein, thereby leading to further aggregation. Conditions associated with alpha synuclein and tau may be referred to as “synucleinopathies” and “tauopathies”, respectively. In some embodiments, the vectored antibody delivery of the present disclosure is utilized to target the aforementioned antigens (e.g., misfolded or aggregated proteins).

AAV Particles and methods of using the AAV particles described in the present disclosure may be used to prevent, manage and/or treat tauopathies or tau associated disease. As a non-limiting example, the AAV particles of the present disclosure comprise a nucleic acid sequence encoding at least one of the sequences described in Table 3.

### V. Kits and Devices

### Kits

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the AAV particles of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569, and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In some embodiments, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point: mounting the guide device relative to the body to move with respect to the PTL, wherein the guide device does not intersect the visualization plane: determining a point of intersection (GPP) between the guide axis and the visualization plane; and aligning the GPP with the sighting point in the visualization plane.

In some embodiments, the AAV particles may be delivered to a subject using a convention-enhanced delivery device. Non-limiting examples of targeted delivery of drugs using convection are described in US Patent Publication Nos. US20100217228, US20130035574, and US 20130035660 and International Patent Publication No. WO2013019830 and WO2008144585, the contents of each of which are herein incorporated by reference in their entireties.

In some embodiments, a subject may be imaged prior to, during and/or after delivery of the AAV particles. The imaging method may be a method known in the art and/or described herein, such as but not limited to, magnetic resonance imaging (MRI). As a non-limiting example, imaging may be used to assess therapeutic effect. As another non-limiting example, imaging may be used for assisted delivery of AAV particles.

In some embodiments, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677, and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In some embodiments, the AAV particles may be delivered using an MRI-compatible tip assembly. Non-limiting examples of MRI-compatible tip assemblies are described in US Patent Publication No. US20140275980, the contents of which is herein incorporated by reference in its entirety.

In some embodiments, the AAV particles may be delivered using a cannula which is MRI-compatible. Non-limiting examples of MRI-compatible cannulas include those taught in International Patent Publication No. WO2011130107, the contents of which are herein incorporated by reference in its entirety.

In some embodiments, the AAV particles may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In some embodiments, the AAV particles may be delivered using a device with an elongated tubular body and a diaphragm as described in US Patent Publication Nos. US20140276582 and US20140276614, the contents of each of which are herein incorporated by reference in their entireties.

In some embodiments, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, the AAV particles may be delivered to a subject using a gene gun.

### VI. Definitions

At various places in the present specification, substituents of compounds of the present disclosure are disclosed in groups or in ranges. It is specifically intended that the present disclosure include each and every individual sub combination of the members of such groups and ranges.

About: As used herein, the term “about” means +/−10% of the recited value.

Adeno-associated virus: The term “adeno-associated virus” or “AAV” as used herein refers to members of the dependovirus genus comprising any particle, sequence, gene, protein, or component derived therefrom.

AA6V Particle: As used herein, an “AAV particle” is a virus which comprises a viral genome with at least one payload region and at least one ITR region. AAV vectors of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions may have activity and this activity may involve one or more biological events.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antibody: As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include V_{H} and VL domains, scFvs, Fab, Fab′, F(ab′)₂, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

Antibody-based composition: As used herein, “antibody-based” or “antibody-derived” compositions are monomeric or multi-meric polypeptides which comprise at least one amino-acid region derived from a known or parental antibody sequence and at least one amino acid region derived from a non-antibody sequence, e.g., mammalian protein.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle. In some embodiments, the term capsid may refer to the nucleic acid encoding the protein shell of the virus particle.

Chimeric antigen receptor (CAR): As used herein, the term “chimeric antigen receptor” or “CAR” refers to an artificial chimeric protein comprising at least one antigen specific targeting region (ASTR), a transmembrane domain and an intracellular signaling domain, wherein the antigen specific targeting region comprises a full-length antibody or a fragment thereof. As a non-limiting example, the ASTR of a CAR may be any of the antibodies listed in Table 3, antibody-based compositions or fragments thereof. Any molecule that is capable of binding a target antigen with high affinity can be used in the ASTR of a CAR. The CAR may optionally have an extracellular spacer domain and/or a co-stimulatory domain. A CAR may also be used to generate a cytotoxic cell carrying the CAR.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T. A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Comprehensive Positional Evolution (CPE™): As used herein, the term “comprehensive positional evolution” refers to an antibody evolution technology that allows for mapping of the effects of amino acid changes at every position along an antibody variable domain's sequence. This comprehensive mutagenesis technology can be used to enhance one or more antibody properties or characteristics.

Comprehensive Protein Synthesis (CPS™): As used herein, the term “comprehensive protein synthesis” refers to a combinatorial protein synthesis technology that can be used to optimize antibody properties or characteristics by combining the best properties into a new, high-performance antibody.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Control Elements: As used herein, “control elements”, “regulatory control elements”, or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to affect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destable”, “destabilize”, or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Epitope: As used herein, an “epitope” refers to a surface or region on a molecule that is capable of interacting with a biomolecule. For example, a protein may contain one or more amino acids, e.g., an epitope, which interacts with an antibody. e.g., a biomolecule. In some embodiments, when referring to a protein or protein module, an epitope may comprise a linear stretch of amino acids or a three-dimensional structure formed by folded amino acid chains.

EvoMap™: As used herein, an EvoMap™ refers to a map of a polypeptide, wherein detailed informatics are presented about the effects of single amino acid mutations within the length of the polypeptide and their influence on the properties and characteristics of that polypeptide.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing): (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle and a delivery agent.

Fragment: A “fragment.” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100/0 of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith. D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov. M. and Devereux. J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., el al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules, such as a V_{H} chain and V_{L} chain or an antibody. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule. Molecules may be modified in many ways including chemically, structurally, and functionally.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid (e.g., viral genome).

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Payload construct: As used herein, “payload construct” or “construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient.” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition: partially or completely delaying onset of one or more symptoms, features, or clinical manifestations of a particular infection, disease, disorder, and/or condition: partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini, 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized.” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” is a polynucleotide comprising at least one inverted terminal repeat (ITR) and at least one encoded payload. A viral genome encodes at least one copy of the payload.

Described herein are compositions, methods, processes, kits and devices for the design, preparation, manufacture and/or formulation of AAV particles. In some embodiments, payloads, such as but not limited to AAV polynucleotides, may be encoded by payload constructs or contained within plasmids or vectors or recombinant adeno-associated viruses (AAVs).

The details of one or more embodiments are set forth in the accompanying description below. Although any materials and methods similar or equivalent to those described herein can be used in the practice or testing of the present disclosure, the preferred materials and methods are now described. Other features, objects and advantages will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### VII. Examples

### Example 1. Generation of Antibodies

### Antibody Production by Hybridoma Technology

Host animals (e.g. mice, rabbits, goats, and llamas) are immunized by an injection with an antigenic protein (e.g., tau) to elicit lymphocytes that specifically bind to the antigen (e.g., tau). Lymphocytes are collected and fused with immortalized cell lines to generate hybridomas. Hybridomas are cultured in a suitable culture medium that is enriched with appropriate selection agents to promote growth.

Antibodies produced by the cultured hybridomas are subjected to analysis to determine binding specificity of the antibodies for the target antigen. Once antibodies with desirable characteristics are identified, corresponding hybridomas are subcloned through limiting dilution procedures and grown by standard methods. Antibodies produced by these cells are isolated and purified using standard immunoglobulin purification procedures.

Tau knock-out mice and/or wild-type mice (CD1 or B6/129) are immunized with a tau antigen using the HT-Hock immunization method. The tau antigens are selected from, (1) ePHF, (2) a mid-domain tau peptide for targeting pathological forms of tau around T181-T217 of human 441 tau, (3) a C-terminal tau peptide for targeting pathological forms of tau around S396-S422, and (4) an AT100 immunopurified tau from human AD brain.

### Recombinant Antibody Production

Recombinant antibodies are produced using heavy and light chain variable region cDNA sequences selected from hybridomas or from other sources. Sequences encoding antibody variable domains expressed by hybridomas are determined by extracting RNA molecules from antibody-producing hybridoma cells and producing cDNA by reverse transcriptase polymerase chain reaction (PCR). PCR is used to amplify cDNA using primers specific for heavy and light chain sequences. PCR products are then subcloned into plasmids for sequence analysis. Antibodies are produced by insertion of resulting variable domain sequences into expression vectors.

Recombinant antibodies are also produced using phage display technology. Target antigens are screened, in vitro, using phage display libraries having millions to billions of phage particles expressing unique single chain variable fragments (scFvs) on their viral coat. Precipitated phage particles are analyzed and sequences encoding expressed scFvs are determined. Sequences encoding antibody variable domains and/or CDRs are inserted into expression vectors for antibody production.

Recombinant antibodies are further produced using yeast surface display technology, wherein antibody variable domain sequences are expressed on the cell surface of Saccharomyces cerevisiae. Recombinant antibodies are developed by displaying the antibody fragment of interest as a fusion to e.g. Aga2p protein on the surface of the yeast, were the protein interacts with proteins and small molecules in a solution, scFvs with affinity towards desired receptors are isolated from the yeast surface using magnetic separation and flow cytometry. Several cycles of yeast surface display and isolation will be done to attain scFvs with desired properties through directed evolution.

### Reverse Translation for Antibody Generation

Nucleotide sequences are derived from antibody amino acid sequences. The amino acid sequence is reverse translated from a parent antibody sequence (e.g., light chain variable or heavy chain variable region) to generate a nucleotide sequence. The nucleotide sequence is then cloned into an antibody constant region framework and tested for expression and activity.

### Codon-Optimization

Nucleotide sequences are codon-optimized to enhance one or more desirable features of the encoded antibody (e.g., expression or affinity for the target). Each antibody nucleotide sequence is codon-optimized according to several methods known in the art and compared to the other codon-optimized variants in vitro or in vivo. All codon-optimized variants are then cloned into viral genomes and tested in vitro. The codon-optimized variants with the greatest in vitro or in vivo expression and/or affinity characteristics are selected for further study.

### Generation of Antibody Fragments

In addition to generating full length anti-tau antibodies, antibody fragments and variants are also generated. These fragments may lack the effector (Fc) region typically included in a full-length antibody. For each anti-tau antibody (e.g., IPN002, PT3, C10.2, CP13), a series of Fab, F(ab)2 and/or scFv fragments are generated and tested in vitro or in vivo. Those showing the best expression in vitro and/or in vivo are selected for further studies.

### Antibody Screening

Anti-tau antibodies that are generated by any of the methods described above or other methods known in the art, are screened for expression and affinity characteristics. Antibodies are screened first with one or more ELISA assays (phospho-tau ELISA, wild-type tau ELISA, or ePHF ELISA). An in vitro cellular screen using a biosensor system is used to assess in vitro seeding. The biosensor system may be a semi-quantitative FRET assay. Further in vitro characterization will be carried out by standard techniques known in the art, such as western blotting or immunoprecipitation, immunohistochemistry, immunocytochemistry, immunofluorescence, or an in vitro seeding assay in primary neurons. Propagation and biophysical characteristics are screened with in vitro and/or in vivo methods, which may rely on a tau-seeding and/or propagation model. Anti-tau antibodies may be purified by any method known to one with skill in the art.

### Example 2. Engineering Viral Genomes for the Expression of Anti-Tau Antibodies

Viral genomes were designed for AAV delivery of anti-tau antibodies IPN002, PHF1, MC1, C10.2 or PT3. The ITR to ITR sequences for the viral genomes are given as TAU_ITR1 to TAU_ITR249 as shown in Tables 4-6 and given by SEQ ID NO: 1990-2075 and 2137-2168, 2171-2237 and 2260-2321.

### Viral Genomes for the Expression of IPN002

Sixteen viral genomes were generated for delivery of anti-tau antibody IPN002 (TAU_ITR1 to TAU_ITR15 and TAU_ITR87; SEQ ID NO: 1990-2004 and 2137). The payload regions for these viral genomes were designed to comprise an antibody heavy chain signal sequence (SEQ ID NO: 1740), an antibody heavy chain variable region (SEQ ID NO: 1821), an antibody heavy chain constant region (SEQ ID NO: 1742), a linker region, an antibody light chain signal sequence (SEQ ID NO: 1861), an antibody light chain variable region (SEQ ID NO: 1939), and an antibody light chain constant region (SEQ ID NO: 1864), but not always in this order when read 5′ to 3′. The linker region selected for these viral genomes was either T2A (SEQ ID NO: 1726) or F2A (SEQ ID NO: 1727). For TAU_ITR1 to TAU_ITR8 (SEQ ID NO: 1990-1997), a furin cleavage site (SEQ ID NO: 1724 or 1725) was located immediately 5′ to the T2A or F2A linker.

In designing viral genomes for the expression of anti-tau antibody IPN002, one of four promoters were selected. The ubiquitous CAG promoter (SEQ ID NO: 2080), further comprising a CMVie element (SEQ ID NO: 2081) and a CBA minimal promoter region (SEQ ID NO: 2082) was used for TAU_ITR1 (SEQ ID NO: 1990), TAU_ITR2 (SEQ ID NO: 1991), TAU_ITR9 (SEQ ID NO: 1998) and TAU_ITR10 (SEQ ID NO: 1999). The ubiquitous CB promoter (SEQ ID NO: 2083), was used for TAU_ITR3 (SEQ ID NO: 1992), TAU_ITR4 (SEQ ID NO: 1993), TAU_ITR11 (SEQ ID NO: 2000) and TAU_ITR87 (SEQ ID NO: 2137). The astrocyte targeting GFAP promoter (SEQ ID NO: 2085) was used for TAU_ITR5 (SEQ ID NO: 1994), TAU_ITR6 (SEQ ID NO: 1995), TAU_ITR12 (SEQ ID NO: 2001) and TAU_ITR13 (SEQ ID NO: 2002). A neuronal targeting synapsin promoter (SEQ ID NO: 2086) was used for TAU_ITR7 (SEQ ID NO: 1996), TAU_ITR8 (SEQ ID NO: 1997), TAU_ITR14 (SEQ ID NO: 2003) and TAU_ITR15 (SEQ ID NO: 2004).

The viral genomes designed with CB, GFAP and synapsin promoters further included an ie1 exon region (SEQ ID NO: 2090), an ie1 intron 1 region (SEQ ID NO: 2095), a human beta-globin intron region (SEQ ID NO: 2097) and a human beta-globin exon region (SEQ ID NO: 2093). These four elements were always maintained in this order when read 5′ to 3′.

The 5′ ITR (SEQ ID NO: 2076), the polyadenylation sequence (SEQ ID NO: 2122) and the 3′ ITR (SEQ ID NO: 2078) were retained across all sixteen viral genomes designed for the expression of anti-tau antibody IPN002.

The order of heavy and light chains (including signal sequence, variable region and constant region) was alternated with respect to 5′ to 3′ direction. When read 5′ to 3′, viral genomes encoding heavy chain antibody sequences, a linker region, and light chain antibody sequences (heavy-linker-light) include TAU_ITR1 to TAU_ITR8 (SEQ ID NO: 1990-1997). Viral genomes encoding light chain antibody sequences, a linker region, and heavy chain antibody sequences (light-linker-heavy) when read 5′ to 3′, include TAU_ITR9 to TAU_ITR15 (SEQ ID NO: 1998-2004) and TAU_ITR87 (SEQ ID NO: 2137).

In summary, these 16 viral genomes represent four configurations HF.T2AL, HF.F2AL, LT2AH, and LF2AH, each driven by one of four promoters, CAG, CB6, GFAP or synapsin. The description of these viral genomes, including the individual sequence regions, is laid out in Tables 15-20 above.

### Viral Genomes for the Expression of C10.2

### Codon-Optimized Variants

Ten viral genomes were designed for the expression of anti-tau antibody C10.2 (TAU_ITR16 to TAU_ITR25; SEQ ID NO: 2005-2014). These viral genomes were differentiated from one another by the codon-optimized sequences for C10.2 (with light chain variable regions given by SEQ ID NO: 1921-1930 and heavy chain variable regions given by SEQ ID NO: 1804-1813). Codon-optimization was determined according to one of the following methods: Bioinformatics (TAU_ITR16: SEQ ID NO: 2005), EMBOSS (TAU_ITR17; SEQ ID NO: 2006), GeneInfinity (TAU_ITR18; SEQ ID NO: 2007), Greg Thatcher (TAU_ITR19; SEQ ID NO: 2008), IDT (TAU_ITR20; SEQ ID NO: 2009), In Silico (TAU_ITR21; SEQ ID NO: 2010), Molecular Biology (TAU_ITR22; SEQ ID NO: 2011). N2P (TAU_ITR23; SEQ ID NO: 2012), SnapGene (TAU_ITR24; SEQ ID NO: 2013), or VectorNTI (TAU_ITR25; SEQ ID NO: 2014).

Beyond the antibody light chain variable and heavy chain variable regions, each viral genome included a 5′ ITR (SEQ ID NO: 2076), a CB promoter (SEQ ID NO: 2083), an exon/intron region comprising an ie1 exon 1 region (SEQ ID NO: 2090), an ie1 intron 1 region (SEQ ID NO: 2095), a human beta-globin intron region (SEQ ID NO: 2097) and a human beta-globin exon region (SEQ ID NO: 2093), a light chain antibody signal sequence (SEQ ID NO: 1861), a light chain constant region (SEQ ID NO: 1864), a T2A linker (SEQ ID NO: 1726), a heavy chain signal sequence (SEQ ID NO: 1740), a heavy chain constant region (SEQ ID NO: 1743), a polyadenylation sequence (SEQ ID NO: 2122) and a 3′ ITR (SEQ ID NO: 2078).

Each of these viral genomes was configured in a light-linker-heavy 5′ to 3′ orientation. The description of these viral genomes, including the individual sequence regions, is laid out in Tables 21 and 22 above.

### Promoter and Configuration Variants

Sixteen viral genomes, representing four configurations (HF.T2AL, LT2AH, HF.F2AL, LF2AH), driven by each of four promoters (CAG, CBA, GFAP, synapsin) were generated for delivery of anti-tau antibody C10.2 (TAU_ITR21, TAU_ITR88 to TAU_ITR102: SEQ ID NO: 2010, 2138-2152). The payload regions for these viral genomes were designed to comprise an antibody heavy chain (SEQ ID NO: 1809), a linker region (T2A; SEQ ID NO: 1726 or F2A; SEQ ID NO: 1727), and an antibody light chain (SEQ ID NO: 1926), not always in this order when read 5′ to 3′, depending on configuration. C10.2 antibody sequences were codon-optimized according to the InSilico method.

One of four promoters was selected in designing viral genomes for the expression of anti-tau antibody C10.2. The ubiquitous CAG promoter (SEQ ID NO: 2080), further comprising a CMVie element (SEQ ID NO: 2081) and a CBA minimal promoter region (SEQ ID NO: 2082), was used for TAU_ITR89 (SEQ ID NO: 2139), TAU_ITR93 (SEQ ID NO: 2143), TAU_ITR97 (SEQ ID NO: 2147) and TAU_ITR100 (SEQ ID NO: 2150). The ubiquitous CB promoter (SEQ ID NO: 2083), further comprising a CMVie enhancer (SEQ ID NO: 2087) immediately 5′ to the promoter sequence was used for TAU_ITR88 (SEQ ID NO: 2138), TAU_ITR92 (SEQ ID NO: 2142), TAU_ITR96 (SEQ ID NO: 2146) and TAU_ITR21 (SEQ ID NO: 2010). The astrocyte targeting GFAP promoter (SEQ ID NO: 2085) was used for TAU_ITR90 (SEQ ID NO: 2140), TAU_ITR94 (SEQ ID NO: 2144), TAU_ITR98 (SEQ ID NO: 2148) and TAU_ITR101 (SEQ ID NO: 2151). A neuronal targeting synapsin promoter (SEQ ID NO: 2086) was used for TAU_ITR91 (SEQ ID NO: 2141), TAU_ITR95 (SEQ ID NO: 2145). TAU_ITR99 (SEQ ID NO: 2149) and TAU_ITR102 (SEQ ID NO: 2152).

Viral genomes driven by CB, GFAP and synapsin promoters further comprised an intron/exon region consisting of an ie1 exon region (SEQ ID NO: 2090), an ie1 intron region (SEQ ID NO: 2095), a human beta-globin intron region (SEQ ID NO: 2097) and a human beta-globin exon region (SEQ ID NO: 2093), as read in the 5′ to 3′ direction.

The 5′ ITR (SEQ ID NO: 2076), the polyadenylation sequence (SEQ ID NO: 2122), and the 3′ ITR (SEQ ID NO: 2078) were retained across all sixteen viral genomes designed for the expression of anti-tau antibody C10.2.

The order of heavy and light chains was alternated with respect to 5′ to 3′ direction. Eight viral genomes (TAU_ITR88 to TAU_ITR91; SEQ ID NO: 2138-2141 and TAU_ITR96 to TAU_ITR99; SEQ ID NO: 2146-2149) were designed encoding heavy chain antibody sequences, a T2A or F2A linker region, and light chain antibody sequences (heavy-linker-light), when read 5′ to 3′. In these heavy-linker-light configuration viral genomes, the linker also included a furin cleavage site (SEQ ID NO: 1724 or 1725) immediately 5′ to the linker sequence. The other eight viral genomes (TAU_ITR92 to TAU_ITR95; SEQ ID NO: 2142-2145, TAU_ITR21; SEQ ID NO: 2010, and TAU_ITR100 to TAU_ITR102: SEQ ID NO: 2150-2152), when read 5′ to 3′, were designed to encode light chain antibody sequences, a T2A or F2A linker region (linker only, no furin cleavage site), and heavy chain antibody sequences (light-linker-heavy).

In summary, these 16 viral genomes represent four configurations HF.T2AL, LT2AH, HF.F2AL, LF2AH, each driven by one of four promoters CAG, CB. GFAP or synapsin, for the expression of anti-tau antibody C10.2.

### Viral Genomes for the Expression of PT3

### Codon-Optimized Variants

Ten viral genomes were designed for the expression of anti-tau antibody PT3 (TAU_ITR26 to TAU_ITR35; SEQ ID NO: 2015-2024). These viral genomes were differentiated from one another by the codon-optimized sequences for PT3 (with light chain variable regions given by SEQ ID NO: 1971-1980 and heavy chain variable regions given by SEQ ID NO: 1851-1860). Codon-optimization was determined according to one of the following methods: GeneScript (TAU_ITR26; SEQ ID NO: 2015), SnapGene (TAU_ITR27; SEQ ID NO: 2016). EMBOSS (TAU_ITR28; SEQ ID NO: 2017), Bioinformatics (TAU_ITR29; SEQ ID NO: 2018), NUS (TAU_ITR30; SEQ ID NO: 2019). NUS2 (TAU_ITR31; SEQ ID NO: 2020), GeneInfinity (TAU_ITR32; SEQ ID NO: 2021), IDT (TAU_ITR33; SEQ ID NO: 2022), Bioinformatics2 (TAU_ITR34; SEQ ID NO: 2023), or NUS3 (TAU_ITR35; SEQ ID NO: 2024).

Beyond the antibody light chain variable and heavy chain variable regions, each viral genome included a 5′ ITR (SEQ ID NO: 2077), a CBA promoter (SEQ ID NO: 2084), an exon/intron region comprising an ie1 exon 1 region (SEQ ID NO: 2090), an ie1 intron 1 region (SEQ ID NO: 2095), a human beta-globin intron region (SEQ ID NO: 2097) and a human beta-globin exon region (SEQ ID NO: 2093), a heavy chain antibody signal sequence (SEQ ID NO: 1740), a heavy chain constant region (SEQ ID NO: 1742), a furin cleavage site (SEQ ID NO: 1724), a T2A linker (SEQ ID NO: 1726), a light chain signal sequence (SEQ ID NO: 1861), a light chain constant region (SEQ ID NO: 1864), a polyadenylation sequence (SEQ ID NO: 2123) and a 3′ ITR (SEQ ID NO: 2079). TAU_ITR27 (SEQ ID NO: 2016) had a partial heavy chain antibody signal sequence.

Each of these viral genomes was configured in a heavy-linker-light 5′ to 3′ orientation. The description of these viral genomes, including the individual sequence regions, is laid out in Tables 23 and 24 above.

Nucleotide sequences for the heavy chain and light chains of two codon-optimized variants, encoding anti-tau antibody PT3 were cloned into viral genomes with expression driven by either the CBA or CAG promoters. Four viral genomes were generated, each with an HF.T2AL configuration (TAU_ITR11: SEQ ID NO: 2161, TAU_ITR112; SEQ ID NO: 2162, TAU_ITR119; SEQ ID NO: 2169, TAU_ITR120; SEQ ID NO: 2170). Western blot analysis of PT3 expression from these four viral genome constructs showed the two bands expected to be expressed for each construct tested. Quantification by mouse IgG1 ELISA as a fold change normalized to Lucia indicated enhanced expression using the CAG promoter and the first codon-optimization variant (TAU_ITR120: SEQ ID NO: 2170). Assessment by wild-type tau ELISA showed PT3 binds to wt tau441 at high concentration, while GSK-phosphorylated tau ELISA showed PT3 weakly binds phosphorylated tau441 (GSKed_Tau protein from Signal Chem) and PHF-tau ELISA showed binding patterns of PT3 to PHF-tau substantially similar to those seen with IPN002.

### Promoter and Configuration Variants

Sixteen viral genomes, representing four configurations (HF.T2AL, HF.F2AL, LT2AH, and LF2AH), driven by each of four promoters (CAG. CB, GFAP, synapsin) were generated for delivery of anti-tau antibody PT3 (TAU_ITR103 to TAU_ITR120: SEQ ID NO: 2153-2170). The payload regions for these viral genomes were designed to comprise an antibody heavy chain (SEQ ID NO: 1859), a linker region, and an antibody light chain (SEQ ID NO: 1980), not always in this order when read 5′ to 3′, depending on configuration. PT3 antibody sequences were codon-optimized according to the SnapGene codon format. The linker region selected for these viral genomes was either T2A (SEQ ID NO: 1726) or F2A (SEQ ID NO: 1727).

One of four promoters was selected in designing viral genomes for the expression of anti-tau antibody PT3. The ubiquitous CAG promoter (SEQ ID NO: 2080), further comprising a CMVie element (SEQ ID NO: 2081) and a CBA minimal promoter region (SEQ ID NO: 2082) was used for TAU_ITR104 (SEQ ID NO: 2154), TAU_ITR108 (SEQ ID NO: 2158). TAU_ITR116 (SEQ ID NO: 2166) and TAU_ITR112 (SEQ ID NO: 2162). The ubiquitous CB promoter (SEQ ID NO: 2083) was used for TAU_ITR103 (SEQ ID NO: 2153), TAU_ITR107 (SEQ ID NO: 2157), TAU_ITR111 (SEQ ID NO: 2161) and TAU_ITR115 (SEQ ID NO: 2165). The astrocyte targeting GFAP promoter (SEQ ID NO: 2085) was used for TAU_ITR105 (SEQ ID NO: 2155), TAU_ITR109 (SEQ ID NO: 2159), TAU_ITR113 (SEQ ID NO: 2163) and TAU_ITR117 (SEQ ID NO: 2167). A neuronal targeting synapsin promoter (SEQ ID NO: 2086) was used for TAU_ITR106 (SEQ ID NO: 2156), TAU_ITR110 (SEQ ID NO: 2160), TAU_ITR114 (SEQ ID NO: 2164) and TAU_ITR117 (SEQ ID NO: 2167).

The viral genomes designed with CB, GFAP and synapsin promoters further included an ie1 exon region (SEQ ID NO: 2090), an ie1 intron 1 region (SEQ ID NO: 2095), a human beta-globin intron region (SEQ ID NO: 2097) and a human beta-globin exon region (SEQ ID NO: 2093). These four elements were always maintained in this order when read 5′ to 3′.

The 5′ ITR (SEQ ID NO: 2076), the polyadenylation sequence (SEQ ID NO: 2122) and the 3′ ITR (SEQ ID NO: 2078) were retained across all sixteen viral genomes designed for the expression of anti-tau antibody PT3.

The order of heavy and light chains was alternated with respect to 5′ to 3′ direction. Eight viral genomes (TAU_ITR103 to TAU_ITR106; SEQ ID NO: 2153-2156 and TAU_ITR111 to TAU_ITR114: SEQ ID NO: 2161-2164) were designed encoding heavy chain antibody sequences, a linker region, and light chain antibody sequences (heavy-linker-light), when read 5′ to 3′. In these heavy-linker-light configuration viral genomes, the linker also included a furin cleavage site (SEQ ID NO: 1724 or 1725) immediately 5′ to the T2A or F2A linker. The other eight viral genomes (TAU_ITR107 to TAU_ITR110; SEQ ID NO: 2157-2160 and TAU_ITR115 to TAU_ITR118; SEQ ID NO: 2165-2168), when read 5′ to 3′, were designed to encode light chain antibody sequences, a linker region (T2A or F2A only, no furin cleavage site), and heavy chain antibody sequences (light-linker-heavy).

In summary, these 16 viral genomes represent four configurations HF.T2AL, HF.F2AL, LT2AH, and LF2AH, each driven by one of four promoters CAG, CB6, GFAP or synapsin, for the expression of anti-tau antibody PT3.

Viral genomes for the expression of PHF/Thirty-six viral genomes were designed for the expression of anti-tau antibody PHF1 (TAU_ITR36 to TAU_ITR71; SEQ ID NO: 2025-2060).

Either a 130 or 141 nucleotide 5′ ITR (SEQ ID NO: 2076 or 2077, respectively) was selected, and matched with either a 130 or 141 nucleotide 3′ ITR (SEQ ID NO: 2078 or 2079, respectively). TAU_ITR69 to TAU_ITR71 (SEQ ID NO: 2058-2060) included 130 nucleotide ITRs, while TAU_ITR36 to TAU_ITR68 (SEQ ID NO: 2025-2057) included 141 nucleotide ITRs.

Promoters were selected from one of four, (1) CAG promoter (SEQ ID NO: 2080), further comprising a CMVie element (SEQ ID NO: 2081) and a CBA minimal promoter region (SEQ ID NO: 2082), (2) CBA promoter (SEQ ID NO: 2084), (3) CB6 promoter comprising a CMV enhancer (SEQ ID NO: 2087) and a CB promoter (SEQ ID NO: 2083), and (4) GFAP promoter (SEQ ID NO: 2085).

Viral genomes with CBA (TAU_ITR46 to TAU_ITR68: SEQ ID NO: 2035-2057) or CB6 promoters (TAU_ITR69 and TAU_ITR70; SEQ ID NO: 2058 and 2059) were further designed to incorporate an exon/intron region comprising an ie1 exon 1 region (SEQ ID NO: 2090-2092), an ie1 intron 1 region (SEQ ID NO: 2095), a human beta-globin intron region (SEQ ID NO: 2097-2102) and a human beta-globin exon region (SEQ ID NO: 2093), or an SV40 intron (SEQ ID NO: 2103).

At least one signal sequence was incorporated into each viral genome for the expression of PHF1, such as, a light chain antibody signal sequence (SEQ ID NO: 1862), a heavy chain antibody signal sequence (SEQ ID NO: 1741) or a human growth hormone 2 signal sequence (SEQ ID NO: 2106). As many as four signal sequences were incorporated into a single viral genome (TAU_ITR67; SEQ ID NO: 2056).

Nucleotide sequences encoding anti-tau antibody PHF1 were selected from light chain sequences given by SEQ ID NO: 1956, 1957 or 1962, and heavy chain sequences given by SEQ ID NO: 1838 and 1839. As many as 3 pairs of heavy and light chain sequences were incorporated into single viral genomes (TAU_ITR61; SEQ ID NO: 2050, TAU_ITR63; SEQ ID NO: 2052, TAU_ITR65; SEQ ID NO: 2054, TAU_ITR67; SEQ ID NO: 2056). Heavy and light chain antibody sequences were separated by a linker sequence selected from SG4S (encoded by SEQ ID NO: 1729) (corresponding protein sequence disclosed as SEQ ID NO: 4535), G4S3 (encoded by SEQ ID NO: 1730) (corresponding protein sequence disclosed as SEQ ID NO: 4537), F2A (encoded by SEQ ID NO: 1727), T2A (encoded by SEQ ID NP: 1726). In some cases, these linkers were combined with a furin cleavage site given by SEQ ID NO: 1724.

The order of heavy and light chains was alternated with respect to 5′ to 3′ direction (heavy-linker-light vs light-linker-heavy). When read 5′ to 3′, viral genomes in the heavy-linker-light configuration include TAU_ITR36 to TAU_ITR40 (SEQ ID NO: 2025-2029), TAU_ITR46 to TAU_ITR50 (SEQ ID NO: 2035-2039), TAU_ITR56 to TAU_ITR64 (SEQ ID NO: 2045-2053) and TAU_ITR69 (SEQ ID NO: 2058). Viral genomes designed in the light-linker-heavy configuration when read 5′ to 3′, include TAU_ITR41 to TAU_ITR45 (SEQ ID NO: 2030-2034), TAU_ITR51 to TAU_ITR55 (SEQ ID NO: 2040-2044), TAU_ITR65 to TAU_ITR68 (SEQ ID NO: 2054-2057) and TAU_ITR70 (SEQ ID NO: 2059) and TAU_ITR71 (SEQ ID NO: 2060).

One of four tag sequences was selected for use in these viral genomes, with as many as three tags per single viral genome. The tag sequences are given by SEQ ID NO: 2118-2121 and represent HA, SEKDEL (“SEKDEL” disclosed as SEQ ID NO: 4546) (21 nt and 18 nt) and HIS tags, respectively.

Polyadenylation sequences were selected from either a rabbit beta-globin polyadenylation sequence (SEQ ID NO: 2122), or a human growth hormone polyadenylation sequence (SEQ ID NO: 2123).

A subset of these viral genomes were supplemented with an albumin filler sequence selected from SEQ ID NO: 2125 or 2126.

The description of these viral genomes, including the individual sequence regions, is laid out in Tables 25-32 above.

### Viral Genomes for the Expression of MC1, PHF1 or IPN002

Viral genomes of Table 6 (SEQ ID NO: 2061-2075; TAU_ITR72-TAU_ITR86) were designed for delivery of anti-tau antibodies MC1 (with heavy chain of SEQ ID NO: 1832 and light chain of SEQ ID NO: 1950), PHF1 (with heavy chain of SEQ ID NO: 1838 and light chain of SEQ ID NO: 1962) and IPN002 (with heavy chain of SEQ ID NO: 1820 and light chain of SEQ ID NO: 1940) to cells. The viral genomes include, besides the antibody coding regions, a 5′ ITR (SEQ ID NO: 2076), a CMV enhancer (SEQ ID NO: 2087), a CB promoter (SEQ ID NO: 2083), an SV40 intron (SEQ ID NO: 2103), a rabbit globin polyadenylation sequence (SEQ ID NO: 2122), and a 3′ITR (SEQ ID NO: 2078).

Viral genomes were designed to encode a linker between light and heavy chain antibody sequences. Linker sequences were derived from an internal ribosome entry site (IRES; SEQ ID NO: 1732), foot and mouth disease virus 2A (F2A; SEQ ID NO: 1727), porcine teschovirus-1 virus 2A (P2A; SEQ ID NO: 1728), or G4S5 (SEQ ID NO: 1731) (corresponding protein sequence disclosed as SEQ ID NO: 4538) sequences. For TAU_ITR72 (SEQ ID NO: 2061), TAU_ITR73 (SEQ ID NO: 2062), TAU_ITR77 (SEQ ID NO: 2066), TAU_ITR78 (SEQ ID NO: 2067), TAU_ITR83 (SEQ ID NO: 2072) and TAU_ITR85 (SEQ ID NO: 2074), a furin cleavage site (F: SEQ ID NO: 1725) was introduced immediately 5′ to the linker sequence. The order of heavy and light chains was alternated with respect to 5′ to 3′ direction. When read 5′ to 3′, viral genomes encoding a heavy chain antibody sequence, a linker region, and a light chain antibody sequence (heavy-linker-light) include TAU_ITR72 to TAU_ITR77 (SEQ ID NO: 2061-2066) and TAU_ITR85 (SEQ ID NO: 2074). Viral genomes encoding a light chain antibody sequence, a linker region, and a heavy chain antibody sequence (light-linker-heavy) when read 5′ to 3′, include TAU_ITR78 to TAU_ITR84 (SEQ ID NO: 2067-2073) and TAU_ITR86 (SEQ ID NO: 2075).

The description of these viral genomes, including the individual sequence regions, is laid out in Tables 33-36 above.

The viral genomes for expression of IPN002, C10.2, PT2, PHF1 and MC1 were incorporated into AAV particles using an AAV1, AAV2, or VOY101 capsid.

### Example 3. Production and Purification of AAV Particles

Any of the viral genomes described herein (e.g., those of Example 2) may be encapsulated in an AAV capsid to generate an AAV particle. AAV particles described herein may be produced using methods known in the art, such as, for example, triple transfection or baculovirus mediated virus production. Any suitable permissive or packaging cell known in the art may be employed to produce the particles. Mammalian cells are often preferred. Also preferred are trans-complementing packaging cell lines that provide functions deleted from a replication-defective helper virus, e.g., 293 cells or other Ela trans-complementing cells.

The gene cassette may contain some or all of the parvovirus (e.g., AAV) cap and rep genes. Preferably, however, some or all of the cap and rep functions are provided in trans by introducing a packaging vector(s) encoding the capsid and/or Rep proteins into the cell. Most preferably, the gene cassette does not encode the capsid or Rep proteins. Alternatively, a packaging cell line is used that is stably transformed to express the cap and/or rep genes

Recombinant AAV virus particles are, in some cases, produced and purified from culture supernatants according to the procedure as described in US20160032254, the contents of which are incorporated by reference. Production may also involve methods known in the art including those using 293T cells, sf9 insect cells, triple transfection or any suitable production method.

In some cases, 293T cells (adhesion/suspension) are transfected with polyethyleneimine (PEI) with plasmids required for production of AAV, i.e., AAV2 rep, an adenoviral helper construct and an ITR flanked transgene cassette. The AAV2 rep plasmid also contains the cap sequence of the particular virus being studied. Twenty-four hours after transfection (no medium changes for suspension), which occurs in DMEM/F17 with/without serum, the medium is replaced with fresh medium with or without serum. Three (3) days after transfection, a sample is taken from the culture medium of the 293 adherent cells. Subsequently cells are scraped, or suspension cells are pelleted, and transferred into a receptacle. For adhesion cells, after centrifugation to remove cellular pellet, a second sample is taken from the supernatant after scraping. Next, cell lysis is achieved by three consecutive freeze-thaw cycles (−80 C to 37 C) or adding detergent triton. Cellular debris is removed by centrifugation or depth filtration and sample 3 is taken from the medium. The samples are quantified for AAV particles by DNase resistant genome titration by DNA qPCR. The total production yield from such a transfection is equal to the particle concentration from sample 3 described above.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278).

### Example 4. Development of ELISA Assay to Determine Affinity to ePHF Tau

An assay was developed to determine the affinity of anti-tau antibodies, expressed from various viral genome constructs, for extracellular tau in the form of paired helical filaments (ePHF). The ePHF were first immobilized on a 96-well plate overnight by pre-coating with 1500× of the concentrated PHF tau at 4° C., washed 3 times with PBS then blocked with 3% BSA for 2 hrs at room temperature or overnight at 4° C. Supernatants from suspensions of Expi 293 cells transfected with anti-tau antibody viral genome constructs (TAU_ITR72-TAU_ITR86) were collected and loaded onto the plates. Anti-tau antibody MC1 was diluted in 3% BSA and analyzed separately as a control. Plates were then incubated for 2 hrs at room temperature. Wells were washed 5 times with TBS/0.5% Tween 20 wash buffer, then incubated with 1:5000 dilution of anti-mouse antibody labeled with HRP (Thermo Fisher Scientific, Waltham, Mass.) for 30 min. Plates were then developed by incubating with one-step TMB substrate (Thermo Fisher Scientific, Waltham, Mass.) for 30 min, stopped by 2N H₂SO₄ and read using a BioTek Synergy H1 hybrid reader (BioTek, Winooski, Vt.) at 450 nm. The concentration of anti-tau antibodies, and their affinity for ePHF tau, was determined using a standard curve. Anti-tau antibodies produced using MC1LIRESH (TAU_ITR80), MC1LF2AH (TAU_ITR79), MC1HF.F2AL (TAU_ITR72), MC1LF.F2AH (TAU_ITR78), and MC1HF.P2AL (TAU_ITR73) viral genome constructs showed similar affinity for ePHF tau as the MC1 control. Anti-tau antibodies generated using MC1HIRESL (TAU_ITR75), MC1LP2AH (TAU_ITR82) and MC1LF.P2AH (TAU_ITR83) viral genome constructs demonstrated lower affinity to ePHF tau than control MC1. Data are shown in Table 92 below (construct names are shown without the MC1 prefix).

TABLE 92
ePHF Tau ELISA: OD450 data

| ng/ml | HIRESL | LIRESH | LF2AH | HF.F2AL | LF.F2AH | LP2AH | HFP2AL | LF.P2AH | MC1 |
|---|---|---|---|---|---|---|---|---|---|
| 1000.0 | 1.0 | 1.0 | 1.1 | 1.1 | 1.1 | 0.8 | 0.9 | 0.6 | — |
| 500.0 | 0.7 | 0.9 | 1.0 | 1.0 | 1.0 | 0.7 | 0.9 | 0.4 | — |
| 166.7 | 0.3 | 0.6 | 0.7 | 0.7 | 0.7 | 0.4 | 0.6 | 0.2 | — |
| 55.6 | 0.2 | 0.3 | 0.4 | 0.5 | 0.4 | 0.2 | 0.4 | 0.1 | — |
| 18.5 | 0.1 | 0.2 | 0.2 | 0.3 | 0.2 | 0.1 | 0.2 | 0.1 | — |
| 6.2 | 0.1 | 0.1 | 0.1 | 0.2 | 0.1 | 0.1 | 0.1 | 0.1 | — |
| 2.1 | 0.1 | 0.1 | 0.1 | 0.1 | 0.1 | 0.1 | 0.1 | 0.1 | — |
| 0.7 | 0.1 | 0.1 | 0.1 | 0.1 | 0.1 | 0.1 | 0.1 | 0.1 | — |
| 2558.9 | — | — | — | — | — | — | — | — | 1.2 |
| 1279.4 | — | — | — | — | — | — | — | — | 1.2 |
| 426.5 | — | — | — | — | — | — | — | — | 1.0 |
| 142.2 | — | — | — | — | — | — | — | — | 0.7 |
| 47.4 | — | — | — | — | — | — | — | — | 0.4 |
| 15.8 | — | — | — | — | — | — | — | — | 0.2 |
| 5.3 | — | — | — | — | — | — | — | — | 0.3 |
| 1.8 | — | — | — | — | — | — | — | — | 0.1 |

According to the same assay, MC1LF2AH (TAU_ITR79), MC1HF.F2AL (TAU_ITR72), IPN002LF2AH (TAU_ITR84), IPN002HF.F2AL (TAU_ITR77), PHF-1LF2AH (TAU_ITR86), and PHF-1HF.F2AL (TAU_ITR85) viral genome constructs were expressed in Expi 293 cells, the supernatants collected and expressed antibodies were tested for affinity to ePHF tau. Antibodies generated from the six viral genome constructs tested showed similar affinity for ePHF tau in comparison to their respective control antibodies (MC1. PHF1 and IPN002 antibodies).

### Example 5. ELISA Assay for Detection of Expressed Antibodies

Expi 293 cell culture supernatants from cells expressing anti-tau antibodies were tested by sandwich ELISA to detect and determine concentrations of expressed antibodies. Ninety-six well plates were pre-coated with anti-mouse IgG1 overnight at 4° C. then washed 3 times with PBS and blocked with 3% BSA for 2 hrs at room temperature. Supernatants were diluted in blocking buffer (3% BSA), added to the wells and incubated for 2 hrs at room temperature. Samples were then washed 5 times with TBS/0.5% Tween 20 wash buffer and incubated with 1:5000 dilution of anti-mouse antibody labeled with HRP (Thermo Fisher Scientific, Waltham, Mass.) for 30 min. Plates were developed by incubating with one-step TMB substrate for 30 min, stopped by 2N H₂SO₄ and read using a BioTek Synergy H1 hybrid reader (BioTek, Winooski, Vt.) at 450 nm. The concentration of expressed MC1 anti-tau antibodies was then determined for each construct using a standard curve (see Table 93).

TABLE 93
Concentrations of expressed antibodies

| Construct name | Antibody concentration μg/ml |
|---|---|
| MC1HIRESL | 4.42 |
| MC1LIRESH | 32.29 |
| MC1LF2AH | 10.74 |
| MC1HF.F2AL | 12.10 |
| MC1LF.F2AH | 12.94 |
| MC1LP2AH | 44.12 |
| MC1HF.P2AL | 23.79 |
| MC1LF.P2AH | 46.43 |

Cells expressing MC1LIRESH (TAU_ITR80), MC1LP2AH (TAU_ITR82) and MC1LF.P2AH (TAU_ITR83) viral genome constructs produced the highest concentration of antibodies from transfected cells.

In a subsequent experiment using the same methods, cell supernatants from Expi 293 cells expressing MC1LF2AH (TAU_ITR79), MC1HF.F2AL (TAU_ITR72), PHF1LF2AH (TAU_ITR86), PHF1HF.F2AL (TAU_ITR85), IPN002LF2AH (TAU_ITR84), or IPN002HF.F2AL (TAU_ITR77) viral genome constructs were also assessed for concentrations of expressed antibodies by sandwich ELISA. Antibody concentrations from supernatants tested are presented in Table 94.

TABLE 94
Concentrations of expressed antibodies

| Construct name | Antibody concentration μg/ml |
|---|---|
| MC1LF2AH | 40.4 |
| MC1HF.F2AL | 4.5 |
| PHF1LF2AH | 28.3 |
| PHF1HF.F2AL | 2.9 |
| IPN002LF2AH | 10.2 |
| IPN002HF.F2AL | 1.6 |

Cells expressing MC1LF2AH (TAU_ITR79), PHF1LF2AH (TAU_ITR86) and IPN002LF2AH (TAU_ITR84) viral genome constructs produced the highest concentration of antibodies from transfected cells.

### Example 6. Western Blotting for Anti-Tau Antibody Expression

Anti-tau antibodies expressed using MC1HIRESL (TAU_ITR75), MC1LIRESH (TAU_ITR80), MC1HF2AL (TAU_ITR74), MC1LF2AH (TAU_ITR79), MC1HF.F2AL (TAU_ITR72), MC1LF.F2AH (TAU_ITR78), MC1HP2AL (TAU_ITR76), MC1LP2AH (TAU_ITR82), MC1HF.P2AL (TAU_ITR73), MC1LF.P2AH (TAU_ITR83), and MC1LG4S5H (TAU_ITR81) viral genome constructs were assessed by Western blotting in both small and large volume (30 mL) cell culture experiments. Expi 293 cells expressing MC1HIRESL (TAU_ITR75), MC1LIRESH (TAU_ITR80), MC1 HF2AL (TAU_ITR74), MC1LF2AH (TAU_ITR79), MC1HF.F2AL (TAU_ITR72). MC1LF.F2AH (TAU_ITR78), MC1HP2AL (TAU_ITR76), MC1LP2AH (TAU_ITR82), MC1HF.P2AL (TAU_ITR73). MC1LF.P2AH (TAU_ITR83), and MC1LG4S5H (TAU_ITR81) viral genome constructs were cultured to produce antibody-rich supernatant. After centrifugation, supernatants were collected and two small samples of each were removed and mixed with equal volumes of Laemmli sample buffer. Samples were then boiled at 95° C. for 5 min before loading into two 4-20% polyacrylamide gels along with molecular weight markers. Both gels were run for 1-2 hrs at 100V under reducing or non-reducing conditions. Proteins were then transferred to a nitrocellulose membrane for 2 hr at 4° C. and stained with anti-mouse IgGs. First, membranes were placed in blocking buffer for 1 h at room temperature or overnight at 4° C. followed by incubation with anti-mouse IgG antibodies in blocking buffer overnight at 4° C. The membranes were then washed three times each for 5 min in TBST and incubated with enzyme-labeled secondary antibody in blocking buffer for 1 hr at room temperature. Membranes were washed three times each for 5 min in TBST then developed using a luminescent substrate.

Under both reducing and non-reducing conditions, three viral genome constructs showed limited expression when initially assessed by Western blot. In normal (reducing) conditions, antibody heavy chains usually run at approximately 50 kD, while light chains are evident at 25 kD. In supernatant from cells expressing MC1HF2AL (TAU_ITR74) and MC1LG4S5H (TAU_ITR81) viral genome constructs, only the 25 kD species was evident while in supernatant from cells expressing MC1HP2AL (TAU_ITR76), neither species appeared. The remaining supernatants showed the anticipated 25 and 50 kD species under reducing conditions and several high molecular weight (80-150 kD) bands under non-reducing conditions.

A similar experiment was conducted using MC1LF2AH (TAU_ITR79), MC1HF.F2AL (TAU_ITR72), IPN002LF2AH (TAU_ITR84), IPN002HF.F2AL (TAU_ITR77), PHF-1LF2AH (TAU_ITR86), and PHF-1HF.F2AL (TAU_ITR86) viral genome constructs. Western blot showed the expected 25 kD and 50 kD bands under reducing conditions and high molecular weight triplets under non-reducing conditions, similar to the appropriate controls (MC1, PHF1 and IPN002 antibodies). LF2AH viral genome constructs generated better expression levels for all three antibodies than the HF.F2AL viral genome constructs.

Antibody concentrations from scaled-up culture conditions (30 mL) were determined for select constructs (see Table 95).

TABLE 95
Antibody concentrations from 30 mL cultures

| Construct name | Concentration μg/ml |
|---|---|
| MC1LIRESH | 20.3 |
| MC1LF2AH | 86.2 |
| MC1HF.F2AL | 9.9 |
| MC1LF.F2AH | 14.7 |
| MC1HF.P2AL | 15.9 |

Viral genome construct MC1 LF2AH (TAU_ITR79) yielded the highest concentration of antibody from transfected cells.

### Example 7. Purification of Anti-Tau Antibody Constructs

### Purification of Anti-Tau Antibody MC1

Anti-tau antibodies expressed in large volumes of Expi 293 cells (30 mL) were purified using protein A/G beads. A column was prepared with protein A/G bead resin and washed 3 times with loading buffer. Supernatants were diluted with equal volumes of loading buffer and applied to the column. Unbound proteins were washed through with loading buffer. Elution buffer was added to the column and fractions collected. Fractions containing proteins were identified by absorbance at 280 nm, pooled together, neutralized and run on polyacrylamide gels as described in Example 6. Under reducing conditions, antibodies produced using MC1LIRESH (TAU_ITR80), MC1LF2AH (TAU_ITR79), MC1HF.F2AL (TAU_ITR72), MC1LF.F2AH (TAU_ITR78), and MC1HF.P2AL (TAU_ITR73) viral genome constructs yielded protein bands when examined by Western blotting that were similar to those observed with MC1 control antibody (bands at 25 kD and 50 kD). Under non-reducing conditions, all expressed antibodies generated a triplet set of bands between 80-150 kD, as did the MC1 control.

Purified anti-tau antibodies were then tested for their affinity to ePHF tau by ELISA assay as described in Example 4. Antibodies with the highest affinity for ePHF tau were those produced using MC1LF2AH (TAU_ITR79), MC1HF.F2AL (TAU_ITR72) and MC1LF.F2AH (TAU_ITR78) viral genome constructs. These antibodies all demonstrated affinity for ePHF tau that was similar to that observed with MC1 control antibody.

Purification of anti-tau antibody PT3 Anti-tau antibody PT3 was produced and purified in a IL culture of ExpiCHO cells, as described above. Cells were transfected with expifectamine and maintained in culture. The culture media was harvested and PT3 purified using a Protein G column, then concentrated to 6 mg/ml in 1×PBS with 55 mM NaCl and 0.001% F-68. When examined by SDS-PAGE under reduced conditions, bands at approximately 25 kD and 50 kD were observed. Under non-reducing conditions, bands were evident at approximately 160 kD. The purified PT3 antibody was tested for affinity to ePHF and wild-type tau by ELISA assay and demonstrated a greater affinity for ePHF tau.

### Example 8. Optimization of Viral Genomes for Antibody Expression

### Viral Genomes for IPN002 Antibody Expression

Ten viral genomes (TAU_ITR1 to TAU_ITR4, TAU_ITR9 to TAU_ITR11, TAU_ITR87, TAU_ITR84, and TAU_ITR77) were generated to test the influence of ubiquitous promoters (CB6 vs CAG) and configuration (HF.F2AL vs LF2AH) on IPN002 expression. Characterization included ExpiCHO transfection, mouse IgG1 ELISA, Western blot, Phospho-tau ELISA and wild-type tau ELISA.

ExpiCHO cells were transfected with each of the IPN002 antibody expressing viral genome constructs listed above to generate an antibody-rich supernatant. Samples of supernatant were prepared for analysis by mouse IgG1 ELISA to determine the expression level of anti-tau antibody IPN002 in the ExpiCHO cells. Quantification of IgG1 (μg/mL) and the average fold change when normalized to Lucia, is shown in Table 96 below.

TABLE 96
Quantification of ExpiCHO Transfection with IPN002 constructs

| | | SEQ | | | Norm. | | Avg. |
|---|---|---|---|---|---|---|---|
| | TAU_ITR | ID | | IgG1 | to | Fold | Fold |
| Construct Name | ID | NO: | Lucia | (μg/mL) | Lucia | Change | Change |
| CB6-IPN002-HF.F2AL | TAU_ ITR3 | 1992 | 11503.8 | 4.9 | 0.4 | 2.8 | 2.8 |
| | | | 11224.7 | 4.8 | 0.4 | 2.8 | |
| CAG-IPN002-HF.F2AL | TAU_ITR2 | 1991 | 5859.2 | 5.0 | 0.9 | 5.5 | 5.5 |
| | | | 6533.7 | 5.5 | 0.8 | 5.5 | |
| CB6-IPN002-LF2AH | TAU_ITR11 | 2000 | 11627.9 | 15.4 | 1.3 | 8.5 | 7.8 |
| | | | 18215.9 | 20.0 | 1.1 | 7.0 | |
| CAG-IPN002-LF2AH | TAU_ITR9 | 1998 | 10953.3 | 52.3 | 4.8 | 30.1 | 46.9 |
| | | | 3238.4 | 31.8 | 9.8 | 63.1 | |
| CB6-IPN002-HF.T2AL | TAU_ITR4 | 1993 | 18660.5 | 23.6 | 1.3 | 8.1 | 5.8 |
| | | | 15305.7 | 8.3 | 0.5 | 3.5 | |
| CAG-IPN002-HF.T2AL | TAU_ITR1 | 1990 | 13201.9 | 74.4 | 5.6 | 36.2 | 32.7 |
| | | | 9462.1 | 42.9 | 4.5 | 29.1 | |
| CB6-IPN002-LT2AH | TAU_ITR87 | 2137 | 9198.4 | 58.0 | 6.3 | 40.5 | 31.6 |
| | | | 17042.6 | 60.5 | 3.5 | 22.8 | |
| CAG-IPN002-LT2AH | TAU_ITR10 | 1999 | 16716.9 | 90.6 | 5.4 | 34.8 | 36.2 |
| | | | 14401.1 | 84.4 | 5.9 | 37.6 | |
| CB6-SV40-IPN002-LF2AH | TAU_ITR84 | 2073 | 15274.7 | 8.0 | 0.5 | 3.4 | 3.4 |
| CB6-SV40-IPN002-HF.F2AL | TAU_ITR77 | 2066 | 18471.8 | 2.9 | 0.2 | 1 | 1 |

Samples of supernatant were prepared for analysis by Western blot, as described in Example 6 above. The resultant Western blots were reviewed for the presence of heavy (50 kD) and light chain (25 kD) species, which were evident for all constructs tested.

Two ELISA assays (PHF-1 IgG1 and anti-eTau), as described in Examples 4 and 5, were also performed to determine antibody production from eight of the constructs. Results are shown in Table 97 below. Cell-free supernatant and anti-tau antibody IPN002 were used as negative and positive controls, respectively.

TABLE 97
ELISA for analysis of antibody production

| | TAU_ITR | SEQ | PHF-1 IgG1 | anti-eTau |
|---|---|---|---|---|
| Construct Name | ID | ID NO: | ELISA: ng/ml | ELISA: ng/ml |
| CB6-IPN002-HF.F2AL | TAU_ITR3 | 1992 | 9.9 | 6.8 |
| CAG-IPN002-HF.F2AL | TAU_ITR2 | 1991 | 9.3 | 7.0 |
| CB6-IPN002-LF2AH | TAU_ITR11 | 2000 | 12.4 | 8.8 |
| CAG-IPN002-LF2AH | TAU_ITR9 | 1998 | 14.9 | 10.8 |
| CB6-IPN002-HF.T2AL | TAU_ITR4 | 1993 | 12.4 | 9.0 |
| CAG-IPN002-HF.T2AL | TAU_ITR1 | 1990 | 18.4 | 12.8 |
| CB6-IPN002-LT2AH | TAU_ITR87 | 2137 | 21.5 | 14.6 |
| CAG-IPN002-LT2AH | TAU_ITR10 | 1999 | 19.6 | 13.4 |
| Ctl supernatant | — | — | 0 | 0 |
| IPN002 ctl | — | — | 16.2 | 14.7 |

The anti-tau antibody IPN002 expressed from these viral genome constructs binds both phospho- and wild-type tau, based on ELISA.

### VOY101.IPN002 Promoter and Viral Genome Configuration Studies in Rodents

Four viral genome configurations (HF.T2AL, HF.F2AL, LT2AH, and LF2AH) for the expression of anti-tau antibody IPN002 were matched with each of four promoters (CAG, CB6, GFAP and synapsin), for the generation of 16 viral genome variants as described in Example 2 and outlined in Tables 15-20. To determine the effects of the promoters and viral genome configurations on brain distribution, cellular tropism, and expression levels, the viral genomes were packaged in VOY101 capsids and single stranded AAV particles delivered by intravenous administration to mice.

First, two intravenous administration routes and associated pharmacokinetics were compared using TAU_ITR1 (SEQ ID NO: 1990) packaged in VOY101 delivered to mice. Overall, the pharmacokinetics and biodistribution patterns were substantially similar if ssAAV particles were delivered via tail vein or retro-orbitally. For the purposes of the present disclosure, when animal studies are described, the term intravenous delivery may refer to any means of intravenous delivery, including, but not limited to, tail vein injection or retro-orbital injection.

### Ubiquitous Promoters for IPN002 Expression

First, viral genomes TAU_ITR1 (SEQ ID NO: 1990; CAG-HF.T2AL), TAU_ITR10 (SEQ ID NO: 1999; CAG-LT2AH), TAU_ITR4 (SEQ ID NO: 1993; CB6-HF.T2AL), and TAU_ITR87 (SEQ ID NO: 2137; CB6-LT2AH), utilizing ubiquitous CAG or CB6 promoters and HF.T2AL or LT2AH configurations were assessed. These four viral genomes were packaged into VOY101 AAV particles (single stranded) and formulated in PBS and 0.001% F-68. Vehicle PBS with 0.001% F-68 was used as a control. An intravenous bolus at a dose of 1.4×1013 vg/kg was administered via tail vein to two-month old female C57Bl/6 mice (n=4 per group; 20 total mice). Twenty-eight days later, mice were sacrificed by intracardiac perfusion and tissue samples collected from the olfactory bulb, hippocampus, cortex, thalamus, brain stem, spinal cord, spleen, liver, and skeletal muscle and processed for further analysis. Serum samples were collected daily. The study design is shown in Table 98 below.

TABLE 98
ssVOY101.IPN002 CAG/CB6 promoter study design

| | | SEQ | | | | | | End |
|---|---|---|---|---|---|---|---|---|
| Construct | TAU_ITR | ID | | Dose | Dose | | | of |
| Configuration | ID | NO: | μL | (vg/kg) | (vg) | Route | N | study |
| CAG-IPN002-HF.T2AL | TAU_ITR1 | 1990 | 100 | 1.4E13 | 2.8E11 | IV | 4 | D 28 |
| CAG-IPN002-LT2AH | TAU_ITR10 | 1999 | 100 | 1.4E13 | 2.8E11 | IV | 4 | D 28 |
| CB6-IPN002-HF.T2AL | TAU_ITR4 | 1993 | 100 | 1.4E13 | 2.8E11 | IV | 4 | D 28 |
| CB6-IPN002-LT2AH | TAU_ITR87 | 2137 | 100 | 1.4E13 | 2.8E11 | IV | 4 | D 28 |
| PBS with 0.001% F-68 | — | — | 100 | — | — | IV | 4 | D 28 |

Samples were analyzed by ePHF ELISA or total tau ELISA, immunohistochemistry (NeuN, GFAP, Iba) and/or immunofluorescence or hematoxylin and eosin staining (H&E) and vector genome quantification using droplet digital PCR (ddPCR) normalized to diploid cell number (VG/DC) using the endogenous transferrin receptor gene (TFRC). Secondary readouts included daily cage side observations for 1 week after AAV particle administration and weekly body weight measurements. Steady increases in body weight over the course of the study indicated that no obvious health issues were encountered as a result of treatment. Vector genome quantifications (VG/DC are shown in Table 99 below.

TABLE 99
Vector genome quantification of ssVOY101.IPN002 constructs

| | TAU_ITR1 | TAU_ITR10 | TAU_ITR4 | TAU_ITR87 | Vehicle |
|---|---|---|---|---|---|
| | (CAG-HL) | (CAG-LH) | (CB-HL) | (CB-LH) | (PBS) |
| Hippocampus | 5.0 | 8.1 | 8.7 | 15.3 | 0.0 |
| Cortex | 7.2 | 14.0 | 19.4 | 42.2 | 0.0 |
| Olfactory Bulb | 2.4 | 5.2 | 7.6 | 17.6 | 0.0 |
| Thalamus | 7.9 | 13.9 | 20.4 | 35.7 | 0.0 |
| Brainstem | 8.1 | 14.7 | 21.8 | 36.6 | 0.0 |
| Spinal Cord | 4.9 | 8.0 | 12.6 | 24.8 | 0.0 |
| Liver | 0.5 | 0.9 | 2.0 | 3.1 | 0.0 |
| Skeletal Muscle | 0.1 | 0.2 | 0.5 | 1.7 | 0.0 |
| Spleen | 0.4 | 0.4 | 1.2 | 2.5 | 0.0 |

In all tissues analyzed, including CNS tissues (hippocampus, cortex, olfactory bulb, thalamus, brainstem and spinal cord), TAU_ITR87 (SEQ ID NO: 2137) with configuration CB6-IPN002-LT2AH showed the highest vector genome quantification per diploid cell. One mouse from this group demonstrated significantly low AAV biodistribution and transduction and these data were removed from the analysis. In peripheral tissues (liver, skeletal muscle and spleen), quantification of vector genome distribution per cell was low for each of the 4 constructs tested. Intravenous administration of ssVOY101.IPN002 particles resulted in markedly higher CNS tissue biodistribution, as compared to in peripheral tissues. Higher vector genome levels were detected when driven by the CB6 promoter as opposed to the CAG promoter.

Samples of hippocampus, cortex, olfactory bulb, thalamus, brainstem, spinal cord, liver and serum were subjected to analysis by ePHF ELISA to determine IPN002 expression from each of the four IPN002 viral genomes tested. IPN002 antibody expression was then normalized to the viral genome biodistribution (IPN002/VG). Data are shown in Table 100 below as ng/mg protein (or ng/mL for serum samples) and IPN002/vg (shortened to ab/vg). Data for one mouse were omitted since no vector genomes were detected.

TABLE 100
IPN002 expression from ssVOY101.IPN002 constructs (ng/mg protein or mL)

| | TAU_ITR1 | | TAU_ITR10 | | TAU_ITR4 | | TAU_ITR87 | | Vehicle | |
|---|---|---|---|---|---|---|---|---|---|---|
| | (CAG-HL) | | (CAG-LH) | | (CB-HL) | | (CB-LH) | | (PBS) | |
| | ng/mg | ab/vg | ng/mg | ab/vg | ng/mg | ab/vg | ng/mg | ab/vg | ng/mg | ab/vg |
| Hippocampus | 62.7 | 9.7 | 48.0 | 6.6 | 35.9 | 3.8 | 44.2 | 2.9 | 0.0 | 0.0 |
| Cortex | 106.1 | 12.4 | 62.3 | 4.6 | 54.8 | 2.6 | 22.3 | 0.6 | 0.0 | 0.0 |
| Olfactory Bulb | 72.6 | 23.1 | 44.2 | 9.3 | 35.3 | 5.9 | 27.5 | 1.6 | 0.0 | 0.0 |
| Thalamus | 59.0 | 6.6 | 46.7 | 3.3 | 40.1 | 1.8 | 16.0 | 0.4 | 0.0 | 0.0 |
| Brainstem | 138.5 | 14.9 | 126.8 | 11.1 | 111.9 | 5.4 | 60.5 | 1.7 | 0.0 | 0.0 |
| Spinal Cord | 124.3 | 24.3 | 150.3 | 28.6 | 78.7 | 4.6 | 34.0 | 1.4 | 0.0 | 0.0 |
| Liver | 39.5 | 81.0 | 118.2 | 175.9 | 8.9 | 5.5 | 5.7 | 2.1 | 0.0 | 0.0 |
| Serum (ng/mL) | 3295.6 | — | 16209 | — | 4807.6 | — | 3364.5 | — | 0.0 | 0.0 |

None of the four viral genomes was proven to significantly enhance IPN002 antibody expression, though TAU_ITR4 (SEQ ID NO: 1993; CB6-IPN002-HF.T2AL) showed a trend toward increased IPN002 expression, as compared to expression from the other three viral genomes. Based on IPN002 expression normalized to vector genome data, TAU_ITR1 (SEQ ID NO: 1990; CAG-IPN002-HF.T2AL) showed the highest expression per vector genome.

Immunofluorescent IgG staining of brain tissue collected from mice treated with an IV injection of ssVOY101.IPN002 AAV particles comprising viral genomes encoding IPN002, driven by CAG or CB6 promoters and one of two viral genome configurations (HF.T2AL or LT2AH). showed greater immunofluorescence (i.e., antibody penetration) in tissue of mice treated with heavy-linker-light chain configuration, as compared to light-heavy-linker configuration with the use of both CAG and CB6 promoters. Comparisons of antibody expression from viral genomes with the same genome configurations but different promoters indicated that the CAG promoter drove higher expression in brain tissues, as measured by immunofluorescence. The highest immunofluorescent signal was observed with TAU_ITR1 (SEQ ID NO: 1990: CAG-IPN002-HF.T2AL), which was consistent with the ePHF tau ELISA results.

Co-immunostaining (fluorescent) of brain tissues for IPN002 (IgG) and neuronal (NeuN) or astrocytic (GFAP) markers showed co-localization of IPN002 with NeuN in neurons of the hippocampus, cortex and thalamus, as well as with GFAP in astrocytes of the cortex.

Anti-IgG immunohistochemistry of brain tissue collected from mice 28 days after treatment with an IV bolus injection of ssVOY101.IPN002 AAV particles comprising viral genomes encoding IPN002, driven by CAG or CB6 promoters and one of two viral genome configurations (HF.T2AL or LT2AH), showed broad antibody expression across the mouse CNS, but stronger staining in tissue of mice treated with heavy-linker-light chain configuration, as compared to light-heavy-linker configuration with the use of both CAG and CB6 promoters. Vehicle-treated control animals exhibited low background levels of staining. Expression of IPN002 antibody was observed in a non-cell type specific manner (neurons and astrocytes showed staining) across multiple brain regions, as may be expected when using ubiquitous promoters.

Immunohistochemistry of brain tissue of mice, treated with an IV bolus injection of ssVOY101.IPN002 AAV particles comprising viral genomes encoding IPN002, driven by CAG or CB6 promoters 28 days prior, for IPN002 (IgG) and astrocytic (S100β) markers showed co-localization of IPN002 with S100β positive cells.

Together, these data indicate that CAG and CB6 promoters may be used to effectively drive IPN002 expression in brain tissue of mice. Viral genome configuration heavy-linker-light leads to approximately 2-fold greater IPN002 antibody expression and tissue distribution, as compared to the light-linker-heavy viral genome configuration, regardless of whether the CAG or CB6 promoter is used. When CAG and CB6 promoters were compared, the CAG promoter was observed to drive greater expression of IPN002 as compared to CB6, regardless of the viral genome configuration. The highest expression of IPN002, per vector genome, was observed from TAU_ITR1 (SEQ ID NO: 1990; CAG-IPN002-HF.T2AL).

### Ubiquitous Promoters and T2A or F2A Linkers for IPN002 Expression

In a second study, the effect of using ubiquitous CAG or CB6 promoters for driving expression of IPN002 from two different viral genome configurations (HF.F2AL or LF2AH) was assessed based on measurement of brain distribution, cellular tropism and antibody expression level. Data from this study further enabled comparison of the effects of T2A and F2A cleavage sites on IPN002 antibody expression, distribution and tropism.

Viral genomes TAU_ITR2 (SEQ ID NO: 1991; CAG-HF.F2AL), TAU_ITR9 (SEQ ID NO: 1998: CAG-LF2AH), TAU_ITR3 (SEQ ID NO: 1992; CB6-HF.F2AL), and TAU_ITR11 (SEQ ID NO: 2000; CB6-LF2AH), were packaged, single stranded, into VOY101 AAV particles an, formulated in PBS and 0.001% F-68. Vehicle PBS with 0.001% F-68 was used as a control. An intravenous bolus at a dose of 1.4×10¹³ vg/kg was administered via tail vein to two-month old female C57Bl/6 mice (n=4 per group; 20 total mice). Twenty-eight days later, mice were sacrificed by intracardiac perfusion and tissue samples collected and processed for further analysis. The right half of the brain (including olfactory bulb) and lumbar spinal cord were post-fixed with 4% PFA for immunohistochemistry. The other half of the brain (dissected into hippocampus, cortex, thalamus, brainstem) and cervical and thoracic spinal cord tissues were processed for use in ELISA assays and vector genome quantification by droplet digital PCR normalized to diploid cell number (VG/DC) using the endogenous transferrin receptor gene (TFRC). Peripheral tissues from spleen, liver and skeletal muscle were also collected. Serum samples were collected at two days prior to dosing, and weekly after dosing on days 7, 14, 21 and 28 (terminal day). CSF samples were collected on the terminal day (D28). The study design is shown in Table 101 below.

TABLE 101
ssVOY101.IPN002 CAG/CB6 promoter and linker study design

| | | SEQ | | | | | | End |
|---|---|---|---|---|---|---|---|---|
| Construct | TAU_ITR | ID | | Dose | Dose | | | of |
| Configuration | ID | NO: | μL | (vg/kg) | (vg) | Route | N | study |
| CAG-HF.F2AL | TAU_ITR2 | 1991 | 100 | 1.4E13 | 2.8E11 | IV | 4 | D 28 |
| CAG-LF2AH | TAU_ITR9 | 1998 | 100 | 1.4E13 | 2.8E11 | IV | 4 | D 28 |
| CB6-HF.F2AL | TAU_ITR3 | 1992 | 100 | 1.4E13 | 2.8E11 | IV | 4 | D 28 |
| CB6-LF2AH | TAU_ITR11 | 2000 | 100 | 1.4E13 | 2.8E11 | IV | 4 | D 28 |
| PBS with 0.001% F-68 | — | — | 100 | — | — | IV | 4 | D 28 |

CNS, peripheral and serum samples were analyzed by ePHF ELISA. Brain, olfactory bulb and spinal cord tissues were assessed by immunohistochemistry (NeuN, S100β, IgG), immunofluorescence and/or hematoxylin and eosin staining (H&E). Vector genome quantification using droplet digital PCR (ddPCR) was carried out for samples collected from cortex, hippocampus, anterior olfactory bulb, brain stem, thoracic spinal cord, liver and skeletal muscle. Secondary readouts included daily cage side observations for 1 week after AAV particle administration and weekly body weight measurements. Steady increases in body weight over the course of the study indicated that no obvious health issues were encountered as a result of treatment. Vector genome quantifications (VG/DC) are shown in the Table 102 below.

TABLE 102
Vector genome quantification of ssVOY101.IPN002 constructs

| | TAU_ITR2 | TAU_ITR9 | TAU_ITR3 | TAU_ITR11 | Vehicle |
|---|---|---|---|---|---|
| | (CAG-HL) | (CAG-LH) | (CB6-HL) | CB6-LH) | (PBS) |
| Hippocampus | 7.0 | 7.3 | 17.2 | 16.8 | 0.0 |
| Cortex | 17.4 | 13.8 | 23.5 | 36.0 | 0.0 |
| Olfactory Bulb | 7.1 | 6.9 | 10.0 | 12.5 | 0.1 |
| Thalamus | 20.3 | 18.2 | 34.0 | 42.2 | 0.0 |
| Brainstem | 20.5 | 19.9 | 24.8 | 44.2 | 0.1 |
| Spinal Cord | 11.1 | 9.5 | 16.0 | 26.3 | 0.2 |
| Liver | 5.6 | 2.0 | 6.8 | 5.9 | 0.0 |
| Skeletal Muscle | 0.4 | 0.4 | 0.7 | 1.5 | 0.0 |
| Spleen | 1.7 | 1.6 | 4.3 | 4.2 | 0.1 |

Across the six CNS tissues analyzed, treatment with each of the four viral genome configurations resulted in similar AAV biodistribution, with no statistically significant difference between treatment groups. A trend was seen toward higher AAV genomes per cell in animals treated with CB6 promoter constructs. In peripheral tissues, all 4 viral genome configurations showed low AAV biodistribution, though delivery of viral genomes comprising a CB6 promoter TAU_ITR3 (SEQ ID NO: 1992; CB6-HFF2AL) and TAU_ITR11 (SEQ ID NO: 2000; CB6-LF2AH) exhibited a slight, statistically non-significant trend toward enhanced expression in liver and skeletal muscle. IV administration of each of the four viral genomes tested resulted in greater AAV biodistribution in CNS tissues as compared to peripheral tissues.

Samples of hippocampus, cortex, olfactory bulb, thalamus, brainstem, spinal cord, liver, spleen, skeletal muscle and serum were subjected to analysis by ePHF ELISA to determine IPN002 expression from each of the four cell-type specific IPN002 viral genomes tested. Control studies were performed to confirm the standard curve and determine that the matrix effect is not observed in this ELISA assay. IPN002 antibody expression was then normalized to the viral genome biodistribution (IPN002/VG). Data are shown in Table 103 below as ng/mg protein (or ng/mL for serum samples) and IPN002/vg (shortened to ab/vg).

TABLE 103
IPN002 expression from ssVOY101.IPN002 constructs (ng/mg protein or mL)

| | TAU_ITR2 | | TAU_ITR9 | | TAU_ITR3 | | TAU_ITR11 | | Vehicle | |
|---|---|---|---|---|---|---|---|---|---|---|
| | (CAG-HL) | | (CAG-LH) | | (CB6-HL) | | (CB6-LH) | | (PBS) | |
| | ng/mg | ab/vg | ng/mg | ab/vg | ng/mg | ab/vg | ng/mg | ab/vg | ng/mg | ab/vg |
| Hippocampus | 7.6 | 1.1 | 10.4 | 1.4 | 1.6 | 0.1 | 6.2 | 0.4 | 0.0 | 0.0 |
| Cortex | 8.6 | 0.5 | 11.6 | 0.8 | 3.7 | 0.2 | 10.0 | 0.3 | 0.0 | 0.0 |
| Olfactory Bulb | 5.0 | 0.6 | 13.6 | 2.1 | 1.1 | 0.1 | 6.3 | 0.5 | 0.0 | 0.0 |
| Thalamus | 8.2 | 0.4 | 14.4 | 0.9 | 4.1 | 0.1 | 9.4 | 0.2 | 0.0 | 0.0 |
| Brainstem | 13.9 | 0.7 | 26.2 | 1.4 | 9.6 | 0.4 | 16.3 | 0.4 | 0.0 | 0.0 |
| Spinal Cord | 23.8 | 2.1 | 14.6 | 1.5 | 7.4 | 0.5 | 11.2 | 0.4 | 0.0 | 0.0 |
| Liver | 9.9 | 3.5 | 14.7 | 11.0 | 3.0 | 0.8 | 0.9 | 0.2 | 0.1 | 0.0 |
| Spleen | 11.5 | 19.3 | 10.1 | 12.1 | 0.2 | 0.0 | 0.9 | 0.6 | 0.0 | 0.0 |
| Muscle | 36.7 | 119.3 | 21.9 | 44.2 | 1.8 | 3.5 | 2.0 | 1.9 | 0.0 | 0.0 |

IPN002 expression was evident in all CNS tissues tested an limited expression was seen in the peripheral tissues of mice treated with each of the four viral genomes. Comparison of IPN002 expression from viral genomes of the same configuration other than for the promoter (CAG-HF.F2AL vs. CB6-HF.F2AL) showed greater expression driven by the CAG promoter than the CB6 promoter. The trend for greater IPN002 expression when using the CAG promoter was also evident when assessed per viral genome (normalized to viral genome biodistribution; ab/vg).

In a direct comparison of viral genomes comprising ubiquitous promotors and either a T2A or F2A linker, eight viral genomes were tested (TAU_ITR1 (SEQ ID NO: 1990: CAG-HF.T2AL), TAU_ITR10 (SEQ ID NO: 1999: CAG-LT2AH), TAU_ITR4 (SEQ ID NO: 1993; CB6-HF.T2AL), and TAU_ITR87 (SEQ ID NO: 2137; CB6-LT2AH), TAU_ITR2 (SEQ ID NO: 1991; CAG-HF.F2AL), TAU_ITR9 (SEQ ID NO: 1998: CAG-LF2AH), TAU_ITR3 (SEQ ID NO: 1992; CB6-HF.F2AL), and TAU_ITR11 (SEQ ID NO: 2000: CB6-LF2AH)) by ePHF ELISA (Table 104) for IPN002 antibody expression (ng/mg). Table 105 shows IPN002 antibody expression normalized to viral genome biodistribution (IPN002/VG).

TABLE 104
IPN002 expression from ssVOY101.IPN002 constructs (ng/mg protein)

| | CAG-HL | | CAG-LH | | CB6-HL | |
|---|---|---|---|---|---|---|
| | T2A | F2A | T2A | F2A | T2A | |
| | (TAU_ITR1) | (TAU_ITR2) | (TAU_ITR10) | (TAU_ITR9) | (TAU_ITR4) | |
| SEQ ID NO: | 1990 | 1991 | 1999 | 1998 | 1993 | |
| Hippocampus | 62.7 | 7.6 | 48.0 | 10.4 | 35.9 | |
| Cortex | 106.1 | 8.6 | 62.3 | 11.6 | 54.8 | |
| Olfactory | 72.6 | 5.0 | 44.2 | 13.6 | 35.3 | |
| Bulb | | | | | | |
| Thalamus | 59.0 | 8.2 | 46.7 | 14.4 | 40.1 | |
| Brainstem | 138.5 | 13.9 | 69.6 | 26.2 | 111.9 | |
| Spinal Cord | 124.3 | 23.8 | 150.3 | 14.6 | 78.7 | |
| Serum | 131274 | 87446.7 | 1452.54 | 76889.3 | 26251.2 | |
| (ng/mL) | | | | | | |
| | | CB6-HL | CB6-LH | | | |
| | | F2A | T2A | F2A | Vehicle (PBS) | |
| | | (TAU_ITR3) | (TAU_ITR87) | (TAU_ITR11) | T2A | F2A |
| | SEQ ID NO: | 1992 | 2137 | 2000 | — | — |
| | Hippocampus | 1.6 | 44.2 | 6.2 | 0.0 | 0.0 |
| | Cortex | 3.7 | 22.3 | 10.0 | 0.0 | 0.0 |
| | Olfactory | 1.1 | 27.5 | 6.3 | 0.0 | 0.0 |
| | Bulb | | | | | |
| | Thalamus | 4.1 | 16.0 | 9.4 | 0.0 | 0.0 |
| | Brainstem | 9.6 | 60.5 | 16.3 | 0.0 | 0.0 |
| | Spinal Cord | 7.4 | 34.0 | 11.2 | 0.0 | 0.0 |
| | Serum | 7688.1 | 30185.2 | 1792.3.1 | 0.0 | 0.0 |
| | (ng/mL) | | | | | |

TABLE 105
Normalized IPN002 expression from ssVQY101.IPN002 constructs

| | CAG-HL | | CAG-LH | | CB6-HL | |
|---|---|---|---|---|---|---|
| | T2A | F2A | T2A | F2A | T2A | |
| | (TAU_ITR1) | (TAU_ITR2) | (TAU_ITR10) | (TAU_ITR9) | (TAU_ITR4) | |
| SEQ ID NO: | 1990 | 1991 | 1999 | 1998 | 1993 | |
| Hippocampus | 9.7 | 1.1 | 6.6 | 1.4 | 3.8 | |
| Cortex | 12.4 | 0.5 | 4.6 | 0.8 | 2.6 | |
| Olfactory | 23.1 | 0.6 | 9.3 | 2.1 | 5.9 | |
| Bulb | | | | | | |
| Thalamus | 6.6 | 0.1 | 3.3 | 0.9 | 1.8 | |
| Brainstem | 14.9 | 0.7 | 11.1 | 1.4 | 5.4 | |
| Spinal Cord | 24.3 | 2.1 | 28.5 | 1.5 | 4.6 | |
| | | CB6-HL | CB6-LH | | | |
| | | F2A | T2A | F2A | Vehicle (PBS) | |
| | | (TAU_ITR3) | (TAU_ITR87) | (TAU_ITR11) | T2A | F2A |
| | SEQ ID NO: | 1992 | 2137 | 2000 | — | — |
| | Hippocampus | 0.1 | 2.9 | 0.4 | 0.0 | 0.0 |
| | Cortex | 0.2 | 0.6 | 0.3 | 0.0 | 0.0 |
| | Olfactory | 0.1 | 1.6 | 0.5 | 0.0 | 0.0 |
| | Bulb | | | | | |
| | Thalamus | 0.1 | 0.4 | 0.4 | 0.0 | 0.0 |
| | Brainstem | 0.4 | 1.7 | 0.4 | 0.0 | 0.0 |
| | Spinal Cord | 0.5 | 1.4 | 0.4 | 0.0 | 0.0 |

In CNS tissues of mice, use of the T2A linker, rather than the F2A, led to higher expression of IPN002 from viral genomes comprising cither a CAG or CB36 promoter and having an HFF2AL or LF2AH configuration, as determined by, ELISA. Analysis based on normalization to viral genome biodistribution demonstrated that IPN002 expression was increased when driven by the CAG, rather than the CB6 promoter, and that the heavy-linker-light configuration yielded higher antibody expression than the light-linker-heavy configuration. These findings were supported by measurement of IPN002 antibody levels in serum samples, by ePHF ELISA, which showed higher levels of IPN002 expression when driven by the CAG promoter than by the CB6 promoter. Delivery of viral genomes comprising a T2A linker, rather than an F2A linker, yielded greater serum levels of IPN002.

Anti-IgG immunohistochemistry of brain tissue collected from mice 28 days after treatment with an IV bolus injection of ssVOY101.IPN002 AAV particles comprising viral genomes encoding IPN002, driven by either CAG or CB6 promoters and one of two viral genome configurations (HF.F2AL or LF2AH), showed broad antibody expression across the mouse CNS, but stronger staining in tissue of mice treated with heavy chain-linker-light chain configuration, as compared to light-linker-heavy configuration. Vehicle-treated control animals exhibited low background levels of staining. Expression of IPN002 antibody was observed in a non-cell type specific manner (neurons and astrocytes showed staining) across multiple brain regions, as may be expected when using ubiquitous promoters.

Immunohistochemistry of brain tissue of mice, treated with an IV bolus injection of ssVOY101.IPN102 AAV particles comprising viral genomes encoding IPN002, driven by CAG or CB6 promoters 28 days prior, for IPN002 (IgG) and astrocytic (S100β) markers showed co-localization of IPN002 with S100β positive cells.

Comparison of IPN002 antibody expression in the brain tissue of mice treated with TAU_ITR2 (SEQ ID NO: 1991: CAG-HF.F2AL) and TAU_ITR9 (SEQ ID NO: 1998; CAG-LF2AH) by immunohistochemistry for IPN002 (IgG) and astrocytes (S100β) showed IPN002 expression in several brain regions of mice treated with TAU_ITR2 (SEQ ID NO: 1991; CAG-HF.F2AL), but very little expression of IPN002 in the brain tissue of mice treated with TAU_ITR9 (SEQ ID NO: 1998; CAG-LF2AH). Co-localization of IPN002 (IgG) and S100β was identified. Comparison of viral genomes differing only by linker sequence (e.g., CAG HFT2AL vs CAG-HFF2AL) showed similar staining patterns when assessed by immunohistochemistry.

Comparison of IPN002 antibody expression in the brain tissue of mice treated with TAU_ITR3 (SEQ ID NO: 1992; CB6-HF.F2AL) and TAU_ITR11 (SEQ ID NO: 2000; CB6-LF2AH) by immunohistochemistry for IPN002 (IgG) and astrocytes (S100β) showed IPN002 expression in several brain regions of mice treated with TAU_ITR3 (SEQ ID NO: 1992; CB6-HF.F2AL), but very little expression of IPN002 in the brain tissue of mice treated with TAU_ITR11 (SEQ ID NO: 2000: CB6-LF2AH). Co-localization of IPN002 (IgG) and S100β was identified. Comparison of viral genomes differing only by linker sequence (e.g., CAG HFT2AL vs CAG-HFF2AL) showed similar staining patterns when assessed by immunohistochemistry.

Comparison of IPN002 antibody expression in the brain tissue of mice treated with TAU_ITR2 (SEQ ID NO: 1991; CAG-HF.F2AL) or TAU_ITR3 (SEQ ID NO: 1992: CB6-HF.F2AL) and TAU_ITR1 (SEQ ID NO: 1990: CAG-HT2AL) or TAU_ITR4 (SEQ ID NO: 1993; CB6-HT2AL) by immunohistochemistry for IPN002 (IgG) and astrocytes (S100β) showed enhanced IPN002 expression when driven by the CAG promoter, as opposed to the CB6 promoter.

Co-localization studies based on immunofluorescent staining for IPN002 and NeuN in cortex, hippocampus and thalamus of mice treated with heavy-linker-light viral genomes showed IPN002 expression in neuronal cells (NeuN positive cells) driven by the CAG or CB6 promoters.

Immunohistochemical analysis demonstrated strong IPN002 antibody expression in the CNS of mice treated with viral genomes utilizing a ubiquitous CAG or CB6 promoter. Viral genomes with heavy-linker-light configurations showed increased IPN002 expression as compared to light-linker-heavy configurations.

Taken together, these data indicate that ubiquitous CAG or CB6 promoters may effectively drive IPN002 expression in CNS tissue of mice, with limited concomitant expression in peripheral tissues (e.g., liver, spleen and muscle). High levels of IPN002 were observed in serum samples of treated mice. Expression of IPN002 in CNS and serum, as measured by PHF ELISA was increased when viral genomes comprising the T2A (rather than F2A) linker were used for IPN002 expression.

### Cell-Type Specific Promoters for IPN002 Expression

The effect of using cell-type specific promoters GFAP or synapsin for driving expression of IPN002 from two different viral genome configurations (HF.T2AL or LT2AH) was assessed based on measurement of brain distribution, cellular tropism and expression level.

Viral genomes TAU_ITR6 (SEQ ID NO: 1995; GFAP-HF.T2AL), TAU_ITR13 (SEQ ID NO: 2002: GFAP-LT2AH), TAU_ITR8 (SEQ ID NO: 1997: SYN-HF.T2AL), and TAU_ITR15 (SEQ ID NO: 2004; SYN-LT2AH), were packaged into VOY101 AAV particles and formulated in PBS and 0.001% F-68. Vehicle PBS with 0.001% F-68 was used as a control. An intravenous bolus at a dose of 1.4×10¹³ vg/kg was administered via tail vein to two-month old female C57Bl/6 mice (n=4 per group: 20 total mice). Twenty-eight days later, mice were sacrificed by intracardiac perfusion and tissue samples collected and processed for further analysis. The right half of the brain (including olfactory bulb) and lumbar spinal cord were post-fixed with 4% PFA for immunohistochemistry. The other half of the brain (dissected into hippocampus, cortex, thalamus, brainstem) and cervical and thoracic spinal cord tissues were processed for use in ELISA assays and vector genome quantification by droplet digital PCR normalized to diploid cell number (VG/DC) using the endogenous transferrin receptor gene (TFRC). Peripheral tissues from spleen, liver and skeletal muscle were also collected. Serum samples were collected at two days prior to dosing, and weekly after dosing on days 7, 14, 21 and 28 (terminal day). CSF samples were collected on the terminal day (D28). The study design is shown in Table 106 below.

TABLE 106
ssVOY101.IPN002 GFAP/SYN promoter study design

| | | SEQ | | | | | | End |
|---|---|---|---|---|---|---|---|---|
| Construct | TAU_ITR | ID | | Dose | Dose | | | of |
| Configuration | ID | NO: | μL | (vg/kg) | (vg) | Route | N | study |
| GFAP-HF.T2AL | TAU_ITR6 | 1995 | 100 | 1.4E13 | 2.8E11 | IV | 4 | D 28 |
| GFAP-LT2AH | TAU_ITR13 | 2002 | 100 | 1.4E13 | 2.8E11 | IV | 4 | D 28 |
| SYN-HF.T2AL | TAU_ITR8 | 1997 | 100 | 1.4E13 | 2.8E11 | IV | 4 | D 28 |
| SYN-LT2AH | TAU_ITR15 | 2004 | 100 | 1.4E13 | 2.8E11 | IV | 4 | D 28 |
| PBS with 0.001% F-68 | — | — | 100 | — | — | IV | 4 | D 28 |

CNS, peripheral and serum samples were analyzed by ePHF ELISA. Brain, olfactory bulb and spinal cord tissues were assessed by immunohistochemistry (NeuN. S100b, Iba), immunofluorescence and/or hematoxylin and eosin staining (H&E). Vector genome quantification using droplet digital PCR (ddPCR) was carried out for samples collected from cortex, hippocampus, anterior olfactory bulb, brain stem, thoracic spinal cord, liver and skeletal muscle. Secondary readouts included daily cage side observations for 1 week after AAV particle administration and weekly body weight measurements. Vector genome quantifications (VG/DC) are shown in the Table 107 below.

TABLE 107
Vector genome quantification of ssVOY101.IPN002 constructs

| | TAU_ITR6 | TAU_ITR13 | TAU_ITR8 | TAU_ITR15 | Vehicle |
|---|---|---|---|---|---|
| | (GFAP-HL) | (GFAP-LH) | (SYN-HL) | (SYN-LH) | (PBS) |
| Hippocampus | 8.7 | 21.2 | 13.4 | 10.1 | 0.0 |
| Cortex | 11.6 | 27.0 | 20.0 | 20.5 | 0.0 |
| Olfactory Bulb | 5.5 | 14.2 | 8.4 | 11.6 | 0.0 |
| Thalamus | 14.7 | 40.3 | 23.4 | 20.2 | 0.0 |
| Brainstem | 13.5 | 39.4 | 25.3 | 16.8 | 0.0 |
| Spinal Cord | 7.7 | 22.6 | 13.0 | 13.5 | 0.0 |
| Liver | 0.7 | 6.4 | 3.5 | 2.4 | 0.0 |
| Skeletal Muscle | 0.1 | 0.4 | 0.2 | 0.3 | 0.0 |
| Spleen | 0.3 | 0.7 | 0.2 | 2.0 | 0.0 |

Across the six CNS tissues analyzed, three (GFAP-HF.T2AL, SYN-HF.T2AL and SYN-LT2AH) of the four viral genome configurations resulted in similar AAV biodistribution. A slight increase in CNS biodistribution was seen with TAU_ITR13 (SEQ ID NO: 2002: GFAP-LT2AH), however this difference was not found to be statistically significant. In peripheral tissues, all 4 viral genome configurations showed low AAV biodistribution.

Samples of hippocampus, cortex, olfactory bulb, thalamus, brainstem, spinal cord, liver, spleen, skeletal muscle and serum were subjected to analysis by ePHF ELISA to determine IPN002 expression from each of the four cell-type specific IPN002 viral genomes tested. Control studies were performed to confirm the standard curve and determine that the matrix effect is not observed in this ELISA assay. IPN002 antibody expression was then normalized to the viral genome biodistribution (IPN002/VG). Data are shown in Table 108 below as ng/mg protein (or ng/mL for serum samples) and IPN002/vg (shortened to ab/vg). Data for one mouse were omitted since no vector genomes were detected.

TABLE 108
IPN002 expression from ssVOY101.IPN002 constructs (ng/mg protein or mL)

| | TAU_ITR6 | | TAU_ITR13 | | TAU_ITR8 | | TAU_ITR15 | | Vehicle | |
|---|---|---|---|---|---|---|---|---|---|---|
| | (GFAP-HL) | | (GFAP-LH) | | (SYN-HL) | | (SYN-LH) | | (PBS) | |
| | ng/mg | ab/vg | ng/mg | ab/vg | ng/mg | ab/vg | ng/mg | ab/vg | ng/mg | ab/vg |
| Hippocampus | 47.6 | 5.1 | 20.0 | 0.9 | 40.4 | 3.0 | 11.6 | 0.9 | 0.0 | 0.0 |
| Cortex | 66.5 | 5.5 | 32.8 | 1.3 | 64.9 | 3.3 | 21.0 | 1.1 | 0.0 | 0.0 |
| Olfactory Bulb | 118.3 | 21.5 | 63.4 | 4.7 | 104.0 | 9.2 | 26.9 | 2.7 | 0.0 | 0.0 |
| Thalamus | 32.2 | 2.1 | 26.6 | 0.9 | 69.3 | 3.0 | 20.3 | 1.0 | 0.0 | 0.0 |
| Brainstem | 53.3 | 4.0 | 42.0 | 1.1 | 119.5 | 3.5 | 28.6 | 1.9 | 0.0 | 0.0 |
| Spinal Cord | 29.3 | 3.8 | 24.4 | 1.0 | 123.8 | 7.8 | 27 7 | 2.3 | 0.0 | 0.0 |
| Liver | 8.7 | 12.7 | 18.0 | 3.9 | 0.7 | 0.1 | 0.2 | 0.1 | 0.0 | 0.0 |
| Serum (ng/mL) | 40202.8 | — | 86884.3 | — | 6652.4 | — | 5025.3 | — | 0.0 | 0.0 |
| Spleen | 9.8 | 83.3 | 5.2 | 8.7 | 0.7 | 3.6 | 0 | 0 | 0.0 | 0.0 |
| Skeletal muscle | 8.3 | — | 7.5 | — | 0.9 | — | 0.3 | — | 0.0 | 0.0 |

IPN002 expression was limited in the peripheral tissues in samples collected from mice treated with each of the four viral genomes, as compared to expression in CNS tissues. In all CNS tissues tested, the heavy-linker-light viral genome configuration (TAU_ITR6 and TAU_ITR8) yielded greater IPN002 expression than the light-linker-heavy viral genome configuration (TAU_ITR13 and TAU_ITR15). This result is consistent with the findings of the study comparing ubiquitous CAG and CB6 promoters.

Comparison of IPN002 antibody expression in the brain tissue of mice treated with TAU_ITR6 (SEQ ID NO: 1995; GFAP-HF.T2AL) and TAU_ITR13 (SEQ ID NO: 2002; GFAP-LT2AH) by immunohistochemistry for IPN002 (IgG) and astrocytes (S100β) showed IPN002 expression in several brain regions of mice treated with TAU_ITR6 (SEQ ID NO: 1995; GFAP-HF.T2AL), but very little expression of IPN002 in the brain tissue of mice treated with TAU_ITR13 (SEQ ID NO: 2002; GFAP-LT2AH). Co-localization of IPN002 (IgG) and S100β was identified.

Immunohistochemical staining for IPN002 (IgG) and S100β in brain tissue of mice treated with TAU_ITR8 (SEQ ID NO: 1997; SYN-HF.T2AL), and TAU_ITR15 (SEQ ID NO: 2004; SYN-LT2AH), showed IPN002 expression in several brain regions of mice treated with TAU_ITR8 (SEQ ID NO: 1997; SYN-HF.T2AL), with IPN002 predominantly expressed in cells with neuronal morphology in the hippocampus, cortex and thalamus. Limited IPN002 expression was evident in brain tissue of mice treated with TAU_ITR15 (SEQ ID NO: 2004; SYN-LT2AH). No co-localization of IPN002 and S100β in these samples was identified.

Immunofluorescence staining of samples collected from mice treated with each of the four vectors confirmed findings that IPN002 expression was readily detected in the tissue of mice treated with TAU_ITR6 (SEQ ID NO: 1995; GFAP-HF.T2AL) or TAU_ITR8 (SEQ ID NO: 1997; SYN-HF.T2AL), but not in tissue of mice treated with TAU_ITR13 (SEQ ID NO: 2002; GFAP-LT2AH) or TAU_ITR15 (SEQ ID NO: 2004; SYN-LT2AH), indicating the importance of a heavy-linker-light viral genome when matched with a cell-type specific promoter such as GFAP or synapsin.

Co-localization studies based on immunofluorescent staining for IPN002 and NeuN showed IPN002 expression in neuronal cells in the tissue of mice treated with heavy-linker-light viral genomes driven by the synapsin promoter. IPN002 expressed from heavy-linker-light viral genomes driven by the GFAP promoter was present in cells with astrocytic morphology.

Immunohistochemical analysis demonstrated strong and appropriate cell specificity of GFAP and synapsin promoters in the brain tissue of treated mice. Further, immunohistochemical results were consistent with those observed by ELISA assay.

Taken together, these data indicate that GFAP and synapsin promoters may effectively drive IPN002 expression in brain tissue of mice and support the previous finding that use of the vector genome configuration HF.T2AL can lead to approximately 2-fold greater IPN002 expression than configuration LT2AH.

### Cell-Type Specific Promoters and 72A or F2A Linkers for IPN002 Expression

In a second study, the effect of using cell-type specific promoters GFAP or synapsin for driving expression of IPN002 from two different viral genome configurations (HF.F2AL or LF2AH) was assessed based on measurement of brain distribution, cellular tropism and antibody expression level. Data from this study further enabled comparison of the effects of T2A and F2A cleavage sites on IPN002 antibody expression, distribution and tropism.

Viral genomes TAU_ITR5 (SEQ ID NO: 1994; GFAP-HF.F2AL), TAU_ITR12 (SEQ ID NO: 2001; GFAP-LF2AH), TAU_ITR7 (SEQ ID NO: 1996; SYN-HF.F2AL), and TAU_ITR14 (SEQ ID NO: 2003; SYN-LF2AH), were packaged, single stranded, into VOY101 AAV particles and formulated in PBS and 0.001% F-68. Vehicle PBS with 0.001% F-68 was used as a control. An intravenous bolus at a dose of 1.4×10¹³ vg/kg was administered via tail vein to two-month old female C57Bl/6 mice (n=4 per group; 20 total mice). Twenty-eight days later, mice were sacrificed by intracardiac perfusion and tissue samples collected and processed for further analysis. The right half of the brain (including olfactory bulb) and lumbar spinal cord were post-fixed with 4% PFA for immunohistochemistry. The other half of the brain (dissected into hippocampus, cortex, thalamus, brainstem) and cervical and thoracic spinal cord tissues were processed for use in ELISA assays and vector genome quantification by droplet digital PCR normalized to diploid cell number (VG/DC) using the endogenous transferrin receptor gene (TFRC). Peripheral tissues from spleen, liver and skeletal muscle were also collected. Serum samples were collected at two days prior to dosing, and weekly after dosing on days 7, 14, 21 and 28 (terminal day). CSF samples were collected on the terminal day (D28). The study design is shown in Table 109 below.

TABLE 109
ssVOY101.1PN002 GFAP/SYN promoter and linker study design

| | | SEQ | | | | | | End |
|---|---|---|---|---|---|---|---|---|
| Construct | TAU_ITR | ID | | Dose | Dose | | | of |
| Configuration | ID | NO: | μL | (vg/kg) | (vg) | Route | N | study |
| GFAP-HF.F2AL | TAU_ITR5 | 1994 | 100 | 1.4E13 | 2.8E11 | IV | 4 | D 28 |
| GFAP-LF2AH | TAU_ITR12 | 2001 | 100 | 1.4E13 | 2.8E11 | IV | 4 | D 28 |
| SYN-HF.F2AL | TAU_ITR7 | 1996 | 100 | 1.4E13 | 2.8E11 | IV | 4 | D 28 |
| SYN-LF2AH | TAU_ITR14 | 2003 | 100 | 1.4E13 | 2.8E11 | IV | 4 | D 28 |
| PBS with 0.001% F-68 | — | — | 100 | — | — | IV | 4 | D 28 |

CNS, peripheral and serum samples were analyzed by ePHF ELISA. Brain, olfactory bulb and spinal cord tissues were assessed by immunohistochemistry (NeuN, S100β, IgG), immunofluorescence and/or hematoxylin and eosin staining (H&E). Vector genome quantification using droplet digital PCR (ddPCR) was carried out for samples collected from cortex, hippocampus, anterior olfactory bulb, brain stem, thoracic spinal cord, liver and skeletal muscle. Secondary readouts included daily cage side observations for 1 week after AAV particle administration and weekly body weight measurements. Vector genome quantifications (VG/DC) are shown in the Table 110 below.

TABLE 110
Vector genome quantification of ssVOY101.IPN002 constructs

| | TAU_ITR5 | TAU_ITR12 | TAU_ITR7 | TAU_ITR14 | Vehicle |
|---|---|---|---|---|---|
| | (GFAP-HL) | (GFAP-LH) | (SYN-HL) | (SYN-LH) | (PBS) |
| Hippocampus | 8.4 | 11.3 | 14.7 | 17.0 | 0.0 |
| Cortex | 17.7 | 22.8 | 29.2 | 32.1 | 0.0 |
| Olfactory Bulb | 5.8 | 9.8 | 11.8 | 13.7 | 0.0 |
| Thalamus | 20.1 | 25.9 | 31.8 | 28.5 | 0.0 |
| Brainstem | 21.4 | 22.1 | 32.1 | 36.8 | 0.0 |
| Spinal Cord | 12.0 | 15.5 | 18.9 | 19.1 | 0.0 |
| Liver | 1.8 | 1.4 | 1.1 | 4.9 | 0.0 |
| Skeletal Muscle | 0.6 | 0.9 | 0.8 | 1.0 | 0.0 |
| Spleen | 0.3 | 0.4 | 0.6 | 1.2 | 0.0 |

Across the six CNS tissues analyzed, treatment with each of the four viral genome configurations resulted in similar AAV biodistribution, with no statistically significant difference between treatment groups. In peripheral tissues, all 4 viral genome configurations showed low AAV biodistribution, though delivery of TAU_ITR14 (SEQ ID NO: 2003; SYN-LF2AH) exhibited a slight, statistically non-significant trend toward enhanced expression in liver and skeletal muscle. IV administration of each of the four viral genomes tested resulted in greater AAV biodistribution in CNS tissues as compared to peripheral tissues.

Samples of hippocampus, cortex, olfactory bulb, thalamus, brainstem, spinal cord, liver, spleen, skeletal muscle and serum were subjected to analysis by ePHF ELISA to determine IPN002 expression from each of the four cell-type specific IPN002 viral genomes tested. Control studies were performed to confirm the standard curve and determine that the matrix effect is not observed in this ELISA assay. IPN002 antibody expression was then normalized to the viral genome biodistribution (IPN002/VG). Data are shown in Table 111 below as ng/mg protein (or ng/mL for serum samples) and IPN002/vg (shortened to ab/vg).

TABLE 111
IPN002 expression from ssVOY101.IPN002 constructs (ng/mg protein or inL)

| | TAU_ITR5 | | TAU_ITR12 | | TAU_ITR7 | | TAU_ITR14 | | Vehicle | |
|---|---|---|---|---|---|---|---|---|---|---|
| | (GFAP-HL) | | (GFAP-LH) | | (SYN-HL) | | (SYN-LH) | | (PBS) | |
| | ng/mg | ab/vg | ng/mg | ab/vg | ng/mg | ab/vg | ng/mg | ab/vg | ng/mg | ab/vg |
| Hippocampus | 1.6 | 0.2 | 9.8 | 0.8 | 20.5 | 1.5 | 17.5 | 1.1 | 0.0 | 0.0 |
| Cortex | 1.8 | 0.1 | 8.0 | 0.4 | 5.7 | 0.2 | 7.4 | 0.2 | 0.0 | 0.0 |
| Olfactory Bulb | 1.8 | 0.2 | 10.4 | 0.8 | 14.9 | 1.1 | 19.6 | 1.5 | 0.0 | 0.0 |
| Thalamus | 1.4 | 0.0 | 9.5 | 0.3 | 10.7 | 0.3 | 19.9 | 0.7 | 0.0 | 0.0 |
| Brainstem | 2.7 | 0.1 | 11.3 | 0.5 | 16.7 | 0.6 | 20.1 | 0.6 | 0.0 | 0.0 |
| Spinal Cord | 4.2 | 0.3 | 7.0 | 0.4 | 20.7 | 1.2 | 12.6 | 0.7 | 0.0 | 0.0 |
| Liver | 1.1 | 1.0 | 2.0 | 2.2 | 0.0 | 0.0 | 0.2 | 0.0 | 0.0 | 0.0 |
| Serum (ng/mL) | 4563.8 | — | 28318 | — | 994.2 | — | 2445.4 | — | 0.0 | 0.0 |
| Spleen | 1.9 | 2.4 | 6.0 | 5.9 | 0.0 | 0.0 | 0.0 | 0.0 | 0.0 | 0.0 |
| Skeletal muscle | 3.0 | 10.6 | 6.5 | 15.6 | 0.2 | 0.3 | 0.2 | 0.3 | 0.0 | 0.0 |

IPN002 expression was evident in all CNS tissues tested and limited expression was seen in the peripheral tissues of mice treated with each of the four viral genomes. In this study, there was no clear pattern for enhanced antibody expression with one particular viral genome configuration as compared to the others, as was seen in the prior experiment using a T2A cleavage site.

In a direct comparison of viral genomes comprising cell-specific promotors and either a T2A or F2A linker, eight viral genomes were tested (TAU_ITR6 (SEQ ID NO: 1995; GFAP-HF.T2AL), TAU_ITR13 (SEQ ID NO: 2002; GFAP-LT2AH) TAU_ITR8 (SEQ ID NO: 1997; SYN-HF.T2AL), and TAU_ITR1 (SEQ ID NO: 2004 SYN-LT2AH), TAU_ITR5 (SEQ ID NO: 1994; GFAP-HF.F2AL) TAU_ITR12 (SEQ ID NO: 2001; GFAP-LF2AH) TAU_ITR7 (SEQ ID NO: 1996m SYN-HF.F2AL), and TAU_ITR14 (SEQ ID NO: 2003. SYN-LF2AH)) by IgG (Table 112) and ePHF ELISA (Table 113) for IPN002 antibody expression (ng/mg). Table 114 shows IPN002 antibody expression normalized to viral genome biodistribution (IPN002/VG)

TABLE 112
IPN002 expression from ssVOY101.IPN002 constructs (ng/mg protein)

| | GFAP-HL | | GFAP-LH | | SYN-HL |
|---|---|---|---|---|---|
| | T2A | F2A | T2A | F2A | T2A |
| | (TAU_ITR6) | (TAU_ITR5) | (TAU_ITR13) | (TAU_ITR12) | (TAU_ITR8) |
| SEQ ID NO: | 1995 | 1994 | 2002 | 2001 | 1997 |
| Hippocampus | 90.5 | 16.0 | 128.5 | 22.4 | 98.2 |
| Cortex | 112.0 | 15.8 | 67.5 | 24.8 | 71.5 |
| Olfactory | 150.7 | 30.1 | 123.9 | 58.0 | 161.1 |
| Bulb | | | | | |

| | SYN-HL | SYN-LH | | Vehicle | |
|---|---|---|---|---|---|
| | F2A | T2A | F2A | (PBS) | |
| | (TAU_ITR7) | (TAU_ITR15) | (TAU_ITR14) | T2A | F2A |
| SEQ ID NO: | 1996 | 2004 | 2003 | — | — |
| Hippocampus | 30.3 | 66.5 | 28.1 | 0.0 | 0.0 |
| Cortex | 17.6 | 46.5 | 26.3 | 10.9 | 4.3 |
| Olfactory | 48.5 | 17.2 | 51.3 | 0.0 | 7.9 |
| Bulb | | | | | |

TABLE 113
IPN002 expression from ssVOY101.IPN002 constructs (ng/mg protein)

| | GFAP-HL | | GFAP-LH | | SYN-HL |
|---|---|---|---|---|---|
| | T2A | F2A | T2A | F2A | T2A |
| | (TAU_ITR6) | (TAU_ITR5) | (TAU_ITR13) | (TAU_ITR12) | (TAU_ITR8) |
| SEQ ID NO: | 1995 | 1994 | 2002 | 2001 | 1997 |
| Hippocampus | 47.6 | 1.6 | 20.0 | 9.8 | 30.3 |
| Conex | 66.5 | 1.8 | 32.9 | 8.0 | 48.7 |
| Olfactory | 118.3 | 1.8 | 63.4 | 10.4 | 78.0 |
| Bulb | | | | | |
| Thalamus | 32.2 | 1.4 | 26.6 | 9.5 | 60.0 |
| Brainstem | 53.3 | 2.7 | 42.0 | 11.3 | 89.6 |
| Spinal Cord | 29.3 | 4.2 | 24.2 | 1.5 | 92.9 |
| Serum | 39769.8 | 4563.8 | 94211.6 | 28318 | 5861.8 |
| (ng/mL) | | | | | |

| | SYN-HL | SYN-LH | | Vehicle | |
|---|---|---|---|---|---|
| | F2A | T2A | F2A | (PBS) | |
| | (TAU_ITR7) | (TAU_ITR15) | (TAU_ITR14) | T2A | F2A |
| SEQ ID NO: | 1996 | 2004 | 2003 | — | — |
| Hippocampus | 20.5 | 11.6 | 17.5 | 0.0 | 0.0 |
| Conex | 5.7 | 21.0 | 7.4 | 0.0 | 0.0 |
| Olfactory | 14.9 | 26.9 | 19.6 | 0.0 | 0.0 |
| Bulb | | | | | |
| Thalamus | 10.7 | 20.3 | 19.9 | 0.0 | 0.0 |
| Brainstem | 16.7 | 28.6 | 20.1 | 0.0 | 0.0 |
| Spinal Cord | 20.7 | 27.7 | 12.6 | 0.0 | 0.0 |
| Serum | 994.2 | 456.3.3 | 2445.4 | 0.0 | 0.0 |
| (ng/mL) | | | | | |

TABLE 114
Normalized IPN002 expression from ssVOY101.IPN002 constructs

| | GFAP-HL | | GFAP-LH | | SYN-HL |
|---|---|---|---|---|---|
| | T2A | F2A | T2A | F2A | T2A |
| | (TAU_ITR6) | (TAU_ITR5) | (TAU_ITR13) | (TAU_ITR12) | (TAU_ITR8) |
| SEQ ID NO: | 1995 | 1994 | 2002 | 2001 | 1997 |
| Hippocampus | 5.1 | 0.2 | 0.9 | 0.8 | 3.0 |
| Cortex | 5.5 | 0.1 | 1.3 | 0.4 | 3.7 |
| Olfactory | 21.5 | 0.2 | 4.7 | 0.8 | 9.2 |
| Bulb | | | | | |
| Thalamus | 2.1 | 0.0 | 0.9 | 0.3 | 2.4 |
| Brainstem | 4.0 | 0.1 | 1.1 | 0.5 | 3.5 |
| Spinal Cord | 3.8 | 0.3 | 1.0 | 0.4 | 5.9 |

| | SYN-HL | SYN-LH | | Vehicle | |
|---|---|---|---|---|---|
| | F2A | T2A | F2A | (PBS) | |
| | (TAU_ITR7) | (TAU_ITR15) | (TAU_ITR14) | T2A | F2A |
| SEQ ID NO: | 1996 | 2004 | 2003 | — | — |
| Hippocampus | 1.5 | 0.9 | 1.1 | 0.0 | 0.0 |
| Cortex | 0.2 | 1.1 | 0.2 | 0.0 | 0.0 |
| Olfactory | 1.1 | 2.7 | 1.5 | 0.0 | 0.0 |
| Bulb | | | | | |
| Thalamus | 0.3 | 1.0 | 0.7 | 0.0 | 0.0 |
| Brainstem | 0.6 | 1.9 | 0.6 | 0.0 | 0.0 |
| Spinal Cord | 1.2 | 1.0 | 0.7 | 0.0 | 0.0 |

In general, use of the T2A linker, rather than the F2A, led to higher expression of IPN002 from viral genomes comprising either a GFAP or synapsin promoter and having an HFF2AL or LF2AH configuration, as determined by ELISA. Serum levels of IPN002 antibody were greater with use of the GFAP promoter rather than the synapsin promoter and were greater when the T2A linker, rather than F2A linker, was used.

Comparison of IPN002 antibody expression in the brain tissue of mice treated with TAU_ITR5 (SEQ ID NO: 1994; GFAP-HF.F2AL) and TAU_ITR12 (SEQ ID NO: 2001; GFAP-LF2AH) by immunohistochemistry for IPN002 (IgG) and astrocytes (S100β) showed IPN002 expression in several brain regions of mice treated with TAU_ITR5 (SEQ ID NO: 1994; GFAP-HF.F2AL), but very little expression of IPN002 in the brain tissue of mice treated with TAU_ITR12 (SEQ ID NO: 2001; GFAP-LF2AH). Co-localization of IPN002 (IgG) and S100β was identified. These findings are very similar to those seen with the use of a T2A linker, above.

Immunohistochemical staining for IPN002 (IgG) and S100β in brain tissue of mice treated with TAU_ITR7 (SEQ ID NO: 1996; SYN-HF.F2AL), and TAU_ITR14 (SEQ ID NO: 2003; SYN-LF2AH), showed IPN002 expression in several brain regions of mice treated with TAU_ITR7 (SEQ ID NO: 1996; SYN-HF.F2AL), with IPN002 predominantly expressed in cells with neuronal morphology in the hippocampus, cortex and thalamus. Limited IPN002 expression was evident in brain tissue of mice treated with TAU_ITR14 (SEQ ID NO: 2003; SYN-LF2AH). No co-localization of IPN002 and S100β in these samples was identified. These findings are very similar to those seen with the use of a T2A linker, above.

Co-localization studies based on immunofluorescent staining for IPN002 and NeuN in cortex, hippocampus (CA subfields) and thalamus of mice treated with heavy-linker-light viral genomes showed IPN002 expression in neuronal cells driven by the synapsin promoter. IPN002 expressed from heavy-linker-light viral genomes driven by the GFAP promoter was present in cells with astrocytic morphology.

Immunohistochemical analysis demonstrated strong and appropriate cell specificity of GFAP and synapsin promoters in the brain tissue of treated mice. Viral genomes with heavy-linker-light configurations showed a trend toward increased IPN002 expression as compared to light-linker-heavy configurations.

Taken together, these data indicate that GFAP and synapsin promoters may effectively drive IPN002 expression in CNS tissue of mice, with limited concomitant expression in peripheral tissues (e.g., liver, spleen and muscle). Cell-type specific promoters (GFAP or synapsin) resulted in IPN002 expression levels comparable to those seen when expression was driven by ubiquitous promoters. High levels of IPN002 were observed in serum samples, particularly in serum of mice treated with ssVOY101.IPN002 using the GFAP promoter, as opposed to synapsin promoter. Expression of IPN002 in CNS and serum, as measured by PHF ELISA was increased when viral genomes comprising the T2A (rather than F2A) linker were used for IPN002 expression.

### Testing of IPN002 Viral Genomes in Primary Neurons

Primary neuronal cultures were used for testing cell specificity, transduction efficiency and expression of IPN002 using the viral genomes outlined above, encapsulated in Voy101. For preparation of primary cultures, brain tissue was removed from CD1 mice at embryonic day 17 (E17) and meninges removed. The hippocampi and cortices were dissected out and digested with papain. Cells were counted and plated (17,500 cells/well) accordingly.

On the 4^{th} day in vitro (DIV4) cells were transduced with AAV particles (3E6, 1E6, 3.33E5, 1.11E5, 3.7E4 VG/cell) comprising the IPN002 encoding viral genomes or Voy101-GFP as a positive control. Untransduced neurons served as the negative control.

Constructs shown in the table below (Table 115), with IPN002 expression driven by ubiquitous or cell-type specific promoters were tested.

TABLE 115
IPN002 Constructs for in vitro testing in primary cultures

| | SEQ | |
|---|---|---|
| Construct ID | ID NO: | Configuration |
| TAU_ITR1 | 1990 | CAG-IPN002-HFT2AL |
| TAU_ITR10 | 1999 | CAG-IPN002-LT2AH |
| TAU_ITR2 | 1991 | CAG-IPN002-HFF2AL |
| TAU_ITR9 | 1998 | CAG-IPN002-LF2AH |
| TAU_ITR4 | 1993 | CBA-IPN002-HFT2AL |
| TAU_ITR87 | 2137 | CBA-IPN002-LT2AH |
| TAU_ITR11 | 2000 | CBA-IPN002-LF2AH |
| TAU_ITR3 | 1992 | CBA-IPN002-HFF2AL |
| TAU_ITR5 | 1994 | GFAP-IPN002-HFF2AL |
| TAU_ITR12 | 2001 | GFAP-IPN002-LF2AH |
| TAU_ITR6 | 1995 | GFAP-IPN002-HFT2AL |
| TAU_ITR13 | 2002 | GFAP-IPN002-LT2AH |
| TAU_ITR7 | 1996 | SYN-IPN002-HFF2AL |
| TAU_ITR14 | 2003 | SYN-IPN002-LF2AH |
| TAU_ITR8 | 1997 | SYN-IPN002-HFT2AL |
| TAU_ITR15 | 2004 | SYN-IPN002-LT2AH |

Cultures were confirmed to be approximately 60-70% neuronal as measured by NeuN staining, with 5-10% of cells staining positive for GFAP (astrocytes).

On DIV7, the supernatant was collected for analysis by mouse IgG ELISA and the cells fixed with (4% PFA and 4% sucrose in PBS for 15 min, followed by 3 PBS washes) for immunocytochemical experiments. Cells were stained by first blocking with 3% BSA and 0.05% saponin in PBS for 1 hour at room temperature. Primary antibodies for IgG, GFAP, and NeuN were incubated overnight at 4 C in blocking buffer then washed with four PBS washes. Secondary antibodies (1:1000 in blocking buffer) were incubated for 1 hour at room temperature then followed by four washes in PBS. Hoecsht was applied at 1:1000 in PBS at room temperature for 15 min to stain nuclei, then washed four times with PBS. Imaging was done on a BioTek Cytation 5.

As expected, cell-type specific promoters drove expression of IPN002 in the appropriate cell type, meaning that the synapsin promoter drove expression in neuronal cells while the GFAP promoter drove expression in astrocytes. These results are consistent with those of the in vivo studies described above.

The supernatant collected on DIV7 (3 days after transduction) was used with IgG1 mouse uncoated ELISA kit (ThermoFisher) for quantification of antibody/mL (ms IgG ng/mL). Of the viral genomes tested wherein a CAG promoter drove expression of IPN002, use of configuration CAG-IPN002-HFT2AL (TAU_ITR1; SEQ ID NO: 1990) resulted in the greatest expression. Similarly, testing of viral genomes wherein a CBA promoter drove expression of IPN002, use of configuration CBA-IPN002-HFT2AL (TAU_ITR4; SEQ ID NO: 1993) resulted in the greatest expression. These ELISA results were consistent with quantification of the immunocytochemistry for number of cells expressing IPN002/total number of cells. Direct comparison of configurations HFT2AL and LT2AH driven by CAG and CBA promoters, by ELISA and quantification of immunocytochemistry demonstrated the CAG promoter to drive greater expression of IPN002 in cells in primary culture and supported the findings that the HFT2AL configuration resulted in greater expression. Analysis of viral genome constructs wherein IPN002 expression is driven by a synapsin promoter by ELISA and quantification of immunocytochemistry again indicated enhanced expression from the HFT2AL configuration, as compared to LT2AH, HFF2AL and LF2AH configurations.

Studies in primary neuronal cultures supported the findings wherein CAG promoters, heavy-linker-light configurations and T2A linkers each contributed to more robust expression of IPN002 as compared to their correlate, i.e., CB6 promoters, light-linker-heavy configurations and F2A linkers, respectively.

### Testing of PT3 Viral Genomes in Primary Culture

Viral genomes encoding anti-tau antibody PT3 with configuration HFT2AL and one of CAG, CBA, GFAP or synapsin promoters were tested in primary neuronal cultures for testing cell specificity, transduction efficiency and expression of PT3. The viral genome constructs shown in Table 116 below were encapsulated in AAV Voy101 and used to transduce primary neuronal cultures as described above.

TABLE 116
PT3 Constructs for in vitro testing in primary cultures

| | SEQ | |
|---|---|---|
| Construct ID | ID NO: | Configuration |
| TAU_ITR111 | 2161 | CBA-PT3-HFT2AL |
| TAU_ITR112 | 2162 | CAG-PT3-HFT2AL |
| TAU_ITR113 | 2163 | GFAP-PT3-HFT2AL |
| TAU_ITR114 | 2164 | SYN-PT3-HFT2AL |

As expected, cell-type specific promoters drove expression of PT3 in the appropriate cell type, meaning that the synapsin promoter drove expression in neuronal cells while the GFAP promoter drove expression in astrocytes.

Studies of viral genomes expressing PT3 demonstrated that IV dosing using a blood brain barrier penetrant capsid (e.g., VOY101) resulted in widespread vector genome biodistribution and expression of vectorized anti-tau antibody in mouse CNS, using each of the constructs tested. The incorporation of cell-type specific promoters targeted the antibody expression to a cellular subgroup. Use of a synapsin promoter was shown to drive PT3 expression in neuronal cells, while use of a GFAP promoter was shown to drive PT3 expression in astrocytes.

Antibody expression levels were shown to be influenced by the promoter choice and by the configuration of the viral genome. Heavy-linker-light configurations yielded greater PT3 antibody expression than light-heavy-linker configurations. Overall, these studies have shown that IV dosing of ssAAVVOY101.PT3 can effectively deliver vectorized antibody to the CNS of mice for both ubiquitous and cell-type specific antibody expression.

### Viral Genomes for PHF1 Antibody Expression

Five viral genome variants were prepared to assess ITR to ITR and payload configuration effects on PHF1 antibody expression. Promoters, introns and heavy and light chain 5′ to 3′ order were assessed. Constructs tested included TAU_ITR86 (PHF-1 LF2AH), TAU_ITR85 (PHF-1 HF.F2AL), and TAU_ITR70 (PHF-1 LF2AH).

ExpiCHO cells were transfected with each of the PHF1 antibody expressing viral genome constructs to generate an antibody-rich supernatant. Samples of supernatant were prepared for analysis by Western blot, as described in Example 6 above. The resultant Western blots were reviewed for the presence of heavy (50 kD) and light chain (25 kD) species. Two of the three constructs yielded bands representing both heavy and light chain species. TAU_ITR85 gave no evidence of antibody expression, lacking bands at both 50 kD and 25 kD. TAU_ITR70 showed the greatest antibody expression, followed by TAU_ITR86, based on Western blot analysis.

Direct comparison of TAU_ITR70 and TAU_ITR86, comprising different intronic regions, indicated greater antibody expression with the use of human beta-globin intronic segments (TAU_ITR70) as compared to an SV40 intron (TAU_ITR86).

Two ELISA assays (PHF-1 IgG1 and anti-eTau) were also performed to determine antibody production from each of the three constructs. Results are shown in Table 17 below.

TABLE 117
ELISA for analysis of antibody production

| | PHF-1 IgG1 Elisa: | anti-eTau Elisa: |
|---|---|---|
| Construct Name | ng/ml | ng/ml |
| TAU_ITR86 | 15273.0 | 1526.5 |
| TAU_ITR85 | 191.0 | 18.8 |
| TAU_ITR70 | 25678.0 | 4285.0 |

### Example 9. rAAV Production of Anti-Tau Antibodies Using HEK293T Cells

HEK293 cells were transfected with three vectors simultaneously: anti-tau antibody encoding viral genomes; vectors expressing rep and cap genes; and a helper vector to generate rAAV9 products. Vector production was the greatest (highest AAV titer vg/mL) when using MC1LF2AH (TAU_ITR79) and MC1HF.F2AL (TAU_ITR72) viral genomes. These two formats were then utilized to generate rAAV9 particles encoding anti-tau antibodies PHF1 and IPN002.

TABLE 118
Vector production in HEK293T cells (vg/mL)

| | 1st | 2nd | Average |
|---|---|---|---|
| AAV9.LIRESH | 2.23E+10 | 3.00E+10 | 2.62E+10 |
| AAV9.LF2AH | 9.12E+10 | 2.69E+11 | 1.80E+11 |
| AAV9.HF.F2AL | 2.32E+10 | 1.24E+11 | 7.35E+10 |
| AAV9.LF.F2AH | 2.00E+10 | 1.15E+10 | 1.57E+10 |
| AAV9.HF.P2AL | 1.70E+10 | 2.63E+10 | 2.16E+10 |

### Example 10. Construction of Viral Genomes for Expression of Antibody Fragments

Full-length antibodies have certain disadvantages in terms of incorporation into AAV viral genomes and particles, immunogenicity and blood brain barrier penetration, that can be overcome by encoding an antibody fragment instead of the full-length antibody. Antibody fragments (e.g., F(ab′)2, Fab and scFv) can be advantageous in several ways. While retaining their antigen-binding capacity, the smaller size of antibody fragments may allow for better tissue penetration and/or have increased solubility. Use of antibody fragments may also provide the opportunity to engage epitopes which may be hidden. As compared to fill-length antibodies, antibody fragments may have reduced immunogenicity and effector effects. Further, antibody fragments are typically straightforward to modify or engineer to one with skill in the art and may be more amenable to incorporation in AAV viral genomes and particles.

Antibody fragments were designed for incorporation into viral genomes and AAV particles. F(ab′)2, Fab and scFv fragments were designed. Fragments were sequence confirmed and tested in vitro for expression patterns. Western blotting was used for semi-quantitation, followed by ePHF ELISA and wild-type tau441 ELISA for further characterization. Based on these results, lead fragment constructs were selected, incorporated into viral genome constructs and packaged into AAV particles, then tested in vivo.

### Viral Genomes for the Expression of PHF1 Fragments

Four constructs were generated for the expression of PHF-1 single chain variable fragments (scFv). Two viral genome configurations (light-linker-heavy and heavy-linker-light) and human growth hormone-2 (Signal1; SEQ ID NO: 2106) and PHF1 antibody heavy chain (Ab2; SEQ ID NO: 1741) signal sequences were tested for effects on the expression of PHF1 scFv and evaluated by western blot analysis as described in Example 6. These viral genomes encoded a His tag (TAG4; SEQ ID NO: 2121) which was used for labeling with anti-his HRP (1:5000 dilution) in Western blot experiments.

Each of the 4 constructs (TAU_ITR42 (LG4S3H-hGH2sig), TAU_ITR44 (LG4S3H-VLsig), TAU_ITR38 (HG4S3L-hGH2sig) and TAU_ITR40 (HG4S3L-VHsig) was transfected into Expi293 and ExpiCHO cells. Supernatants were collected and analyzed by Western blot. Four replicates were run for each construct. TAU_ITR42 and TAU_ITR40 each had one sample that showed no antibody expression based on Western blot analysis, while each of the other samples yielded a single prominent band indicating scFv expression. TAU_ITR38 and TAU_ITR40 (HG4S3L configuration) showed slightly elevated scFv expression, based on Western blot results.

Supernatants from transfected ExpiCHO and Expi293 cells were also tested for binding affinity to PHF tau, using the ePHF tau ELISA as described in Example 4. An anti-His HRP (1:5000) was used for detection. Untransfected ExpiCHO and Expi293 supernatants were used as control. OD450 data are shown below, in Table 119.

TABLE 119
PHF Tau ELISA: OD450 data

| | Expi293 | | | | ExpiCHO |
|---|---|---|---|---|---|
| dilution | TAU_ITR42 | TAU_ITR44 | TAU_ITR38 | TAU_ITR40 | TAU_ITR42 |
| 1 | 1.4 | 1.5 | 2.9 | 3.0 | 1.3 |
| 4 | 0.5 | 0.6 | 2.7 | 2.7 | 0.7 |
| 16 | 0.3 | 0.3 | 2.1 | 2.1 | 0.4 |
| 64 | 0.2 | 0.2 | 1.6 | 1.5 | 0.2 |
| 256 | 0.1 | 0.1 | 0.8 | 0.7 | 0.1 |
| 1024 | 0.1 | 0.1 | 0.4 | 0.3 | 0.1 |
| 4096 | 0.1 | 0.0 | 0.1 | 0.1 | 0.1 |
| 16384 | 0.1 | 0.0 | 0.1 | 0.1 | 0.0 |
| 65536 | 0.0 | 0.0 | 0.0 | 0.0 | 0.0 |
| 262144 | 0.0 | 0.0 | 0.0 | 0.0 | 0.0 |
| 1048576 | 0.0 | 0.0 | 0.0 | 0.0 | 0.0 |
| 4194304 | 0.1 | 0.0 | 0.0 | 0.0 | 0.0 |

| | | | | Controls | |
|---|---|---|---|---|---|
| | ExpiCHO | | | Expi293 | ExpiCHO |
| dilution | TAU_ITR44 | TAU_ITR38 | TAU_ITR40 | CTL | CTL |
| 1 | 1.4 | 3.1 | 3.0 | 0.0 | 0.1 |
| 4 | 0.7 | 2.9 | 2.8 | 0.0 | 0.0 |
| 16 | 0.4 | 2.6 | 2.5 | 0.0 | 0.0 |
| 64 | 0.3 | 2.1 | 2.0 | 0.0 | 0.0 |
| 256 | 0.1 | 1.4 | 1.3 | 0.0 | 0.0 |
| 1024 | 0.1 | 0.6 | 0.6 | 0.0 | 0.0 |
| 4096 | 0.1 | 0.2 | 0.3 | 0.0 | 0.0 |
| 16384 | 0.0 | 0.1 | 0.1 | 0.0 | 0.0 |
| 65536 | 0.0 | 0.1 | 0.1 | | |
| 262144 | 0.0 | 0.0 | 0.0 | | |
| 1048576 | 0.0 | 0.0 | 0.0 | | |
| 4194304 | 0.0 | 0.0 | 0.0 | | |

PHF1 scFvs generated from TAU_ITR38 and TAU_ITR40 viral genome constructs (both heavy-linker-light configuration) showed greater affinity for ePHF tau than scFvs generated from TAU_ITR42 and TAU_ITR44 viral genome constructs (both light-linker-heavy configuration), based on ePHF tau ELISA assay.

### Viral Genomes for the Expression of IPN002 Fragments

Eighteen viral genomes were designed for the expression of anti-tau antibody IPN002 Fab, F(ab′)2, extended Fab, or scFv fragments, with components as outlined in Table 120 below. One of two promoters, CAG or CBA, was selected. Three configurations were tested (HF.T2AL, LT2AH, scFv), using each of the two promoters. For F(ab′)2 constructs one of two hinge components was also introduced. ScFv constructs alternated heavy chain or light chain first when read 5′ to 3′ and relied upon G4S linkers (“G4S” disclosed as SEQ ID NO: 4535) of variable lengths.

TABLE 120
Viral genomes for IPN002 fragment expression

| | Intron/exon | | | | SEQ |
|---|---|---|---|---|---|
| Promoter | region | Configuration | Description | Construct ID | ID NO: |
| CBA | hGbin | HF.T2AL | F(ab′)2_hIgG3_2hinge | TAU_ITR121 | 2171 |
| | | | F(ab′)2_hIgG3_3hinge | TAU_ITR122 | 2172 |
| | | | Fab | TAU_ITR123 | 2173 |
| | | | Fab_ext | TAU_ITR124 | 2174 |
| CAG | — | HF.T2AL | F(ab′)2_hIgG3_2hinge | TAU_ITR125 | 2175 |
| | | | F(ab′)2_hIgG3_3hinge | TAU_ITR126 | 2176 |
| | | | Fab | TAU_ITR127 | 2177 |
| | | | Fab_ext | TAU_ITR128 | 2178 |
| CBA | hGbin | LT2AH | Fab_ext | TAU_ITR129 | 2179 |
| CAG | — | LT2AH | Fab_ext | TAU_ITR130 | 2180 |
| CBA | hGbin | scFv-HA | H(G4S)3L | TAU_ITR133 | 2183 |
| | | | H(G4S)L | TAU_ITR131 | 2181 |
| | | | L(G4S)3H | TAU_ITR146 | 2196 |
| | | | L(G4S)H | TAU_ITR144 | 2194 |
| CAG | — | scFv-HA | H(G4S)3L | TAU_ITR139 | 2189 |
| | | | H(G4S)L | TAU_ITR137 | 2187 |
| | | | L(G4S)3H | TAU_ITR152 | 2202 |
| | | | L(G4S)H | TAU_ITR150 | 2200 |

Anti-HA western blot of IPN002 scFv-HA viral genome constructs demonstrated bands at approximately 30 kd for each of the 8 constructs tested. Quantification data from anti-HA western blot and wtTau ELISA data are shown in the Table 121 below.

TABLE 121
IPN002 scFv-HA screening results

| | | | | | SEQ | Anti-HA | wtTau |
|---|---|---|---|---|---|---|---|
| | Intron/exon | Config- | | Construct | ID | WS | ELISA |
| Promoter | region | uration | Description | ID | NO: | μg/mL | EC₅₀ |
| CBA | hGbin | scFv-HA | H(G4S)3L | TAU_ITR133 | 2183 | 7.0 | 74.5 |
| | | | H(G4S)L | TAU_ITR131 | 2181 | 2.3 | 423.3 |
| | | | L(G4S)3H | TAU_ITR146 | 2196 | 41.9 | 37.9 |
| | | | L(G4S)H | TAU_ITR144 | 2194 | 2.2 | 93.1 |
| CAG | | scFv-HA | H(G4S)3L | TAU_ITR139 | 2189 | 10.5 | 29.5 |
| | | | H(G4S)L | TAU_ITR137 | 2187 | 3.4 | 56.8 |
| | | | L(G4S)3H | TAU_ITR152 | 2202 | 8.4 | 34.7 |
| | | | L(G4S)H | TAU_ITR150 | 2200 | 4.8 | 232 |

IPN002 F(ab′)2, Fab and Fab_ext constructs were also assessed by western blot and tau ELISA assays. Western blot analysis of the 16 constructs outlined above showed a 30 kD band for all but two constructs. Viral genome contracts TAU_ITR121 (SEQ ID NO: 2171) and TAU_ITR125 (SEQ ID NO: 2175) showed no expression due to a mutation in the antibody encoding sequences. The remaining IPN002 F(ab′)2, Fab and Fab_ext viral genome constructs were assessed by tau ELISA and the results are shown in Tables 122 and 123 below.

TABLE 122
IPN002 HF.T2AL-Fab screening results

| | | | | | SEQ | wtTau | PHF Tau |
|---|---|---|---|---|---|---|---|
| | Intron/exon | Config- | | Construct | ID | ELISA | ELISA |
| Promoter | region | uration | Description | ID | NO: | EC₅₀ | EC₅₀ |
| CBA | hGbin | HF.T2AL | Fab | TAU_ITR123 | 2173 | 0.5 | 0.1 |
| | | | Fab_ext | TAU_ITR124 | 2174 | 0.5 | 0.2 |
| CAG | | HF.T2AL | Fab | TAU_ITR127 | 2177 | 0.5 | 0.1 |
| | | | Fab_ext | TAU_ITR128 | 2178 | 0.6 | 0.1 |
| Cells | — | — | — | — | — | 0.2 | 0.0 |
| IPN002 | — | — | — | — | — | 0.3 | 0.0 |

TABLE 123
IPN002 LT2AFl-Fab screening results

| | | | | | SEQ | wtTau | PHF Tau |
|---|---|---|---|---|---|---|---|
| | Intron/exon | Config- | | Construct | ID | ELISA | ELISA |
| Promoter | region | uration | Description | ID | NO; | EC₅₀ | EC₅₀ |
| CBA | hGbin | LT2AH | Fab_ext | TAU_ITR129 | 2179 | 0.3 | 0.1 |
| CAG | — | LT2AH | Fab_ext | TAU_ITR130 | 2180 | 0.3 | 0.1 |
| Cells | — | — | — | — | — | 65.1 | 11.2 |
| IPN002 | — | — | — | — | — | 0.2 | 0.0 |

These preliminary data indicate that viral genomes designed for expression of IPN002 Fab fragments, generate IPN002 Fabs that have affinity to and bind tau.

IPN002 F(ab)2 constructs were assessed in vitro by western blotting and ELISA. Under non-reduced conditions, each of the four IPN002 F(ab′)2 constructs outlined above, showed bands at approximately 150 kd based on western blot of 293 cell supernatant. In reduced conditions, all four constructs tested demonstrated a band between 25-30 kD. IPN002 F(ab′)2 constructs driven by CBA promoter were then tested by PHF-tau and wild-type tau ELISA, with data shown in the Table 124 below.

TABLE 124
IPN002 CBA-F(ab′)2 screening results

| | | | | | SEQ | wtTau | PHF Tau |
|---|---|---|---|---|---|---|---|
| | Intron/exon | | | Construct | ID | ELISA | ELISA |
| Promoter | region | Config. | Description | ID | NO: | EC₅₀ | EC₅₀ |
| CBA | hGbin | HF.T2AL | F(ab′)2_hIgG3_2hinge | TAU_ITR121 | 2171 | 109.1 | 48.9 |
| | | | F(ab′)2_hIgG3_3hinge | TAU_ITR122 | 2172 | 116.7 | 64.3 |

IPN002 Fab constructs were assessed in vitro by western blotting and ELISA. Under non-reduced conditions, all of the IPN002 Fab constructs tested showed bands at approximately 23 kd based on western blot of 293 cell supernatant. In reduced conditions, all constructs tested demonstrated a band between 25-30 kD. IPN002 Fab constructs were then tested by PHF-tau and wild-type tau ELISA, with data shown in the Table 125 below.

TABLE 125
IPN002 Fab screening results

| | | | | | SEQ | wtTau | PHF Tau |
|---|---|---|---|---|---|---|---|
| | Intron/exon | Config- | | Construct | ID | ELISA | ELISA |
| Promoter | region | uration | Description | ID | NO: | EC₅₀ | EC₅₀ |
| CBA | hGbin | HF.T2AL | Fab | TAU_ITR123 | 2173 | 18.8 | 14.0 |
| | | | Fab_ext | TAU_ITR124 | 2174 | 18.7 | 15.3 |
| CAG | — | HF.T2AL | Fab | TAU_ITR127 | 2177 | 22.9 | 17.0 |
| | | | Fab_ext | TAU_ITR128 | 2173 | 28.6 | 23 |
| CBA | hGbin | LT2AH | Fab_ext | TAU_ITR129 | 2179 | 30.2 | 21.35 |
| CAG | — | LT2AH | Fab_ext | TAU_ITR130 | 2180 | 23.1 | 18.6 |

Results of ELISA assays of IPN002 Fab and F(ab′)2 fragments indicate that both are able to bind wild-type and pathological tau.

Twenty-five IPN002 scFv constructs were made using one of two promoters (CBA-hGbin or CAG), alternating antibody sequence configurations (heavy-linker-light (HL) vs light-linker-heavy (LH)) and flexible G4S linkers (“G4S” disclosed as SEQ ID NO: 4535) of different lengths. The construct design is shown in Table 126 below. In the table below, the amino acid sequence of the linker are: G4S is SEQ ID NO: 4535, (G4S)2 is SEQ ID NO: 4539, (G4S)3 is SEQ ID NO: 4537, (G4S)4 is SEQ ID NO: 4540, (G4S)5 is SEQ ID NO: 4538, (G4S)6 is SEQ ID NO: 4541, and (G4S)8 is SEQ ID NO: 4542.

TABLE 126
IPN002 scFv construct design

| | | | | | | SEQ |
|---|---|---|---|---|---|---|
| Construct | | | Config- | | Construct | ID |
| No. | Promoter | Antibody | uration | Linker | ID | NO: |
| 1 | CBA-hGbin | IPN002 | HL | G4S | TAU_ITR131 | 2181 |
| 2 | CBA-hGbin | IPN002 | HL | (G4S)2 | TAU_ITR132 | 2182 |
| 3 | CBA-hGbin | IPN003 | HL | (G4S)3 | TAU_ITR133 | 2183 |
| 4 | CBA-hGbin | IPN008 | HL | (G4S)4 | TAU_ITR134 | 2184 |
| 5 | CBA-hGbin | IPN004 | HL | (G4S)5 | TAU_ITR135 | 2185 |
| 6 | CBA-hGbin | IPN0011 | HL | (G4S)6 | TAU_ITR136 | 2186 |
| 7 | CAG | IPN002 | HL | G4S | TAU_ITR137 | 2187 |
| 8 | CAG | IPN002 | HL | (G4S)2 | TAU_ITR138 | 2188 |
| 9 | CAG | IPN003 | HL | (G4S)3 | TAU_ITR139 | 2189 |
| 10 | CAG | IPN008 | HL | (G4S)4 | TAU_ITR140 | 2190 |
| 11 | CAG | IPN0010 | HL | (G4S)5 | TAU_ITR141 | 2191 |
| 12 | CAG | IPN0011 | HL | (G4S)6 | TAU_ITR142 | 2192 |
| 13 | CAG | IPN0012 | HL | (G4S)8 | TAU_ITR143 | 2193 |
| 14 | CBA-hGbin | IPN005 | LH | G4S | TAU_ITR144 | 2194 |
| 15 | CBA-hGbin | IPN005 | LH | (G4S)2 | TAU_ITR145 | 2195 |
| 16 | CBA-hGbin | IPN006 | LH | (G4S)3 | TAU_ITR146 | 2196 |
| 17 | CBA-hGbin | IPN007 | LH | (G4S)4 | TAU_ITR147 | 2197 |
| 18 | CBA-hGbin | IPN0012 | LH | (G4S)5 | TAU_ITR148 | 2198 |
| 19 | CBA-hGbin | IPN0013 | LH | (G4S)6 | TAU_ITR149 | 2199 |
| 20 | CAG | IPN004 | LH | G4S | TAU_ITR150 | 2200 |
| 21 | CAG | IPN004 | LH | (G4S)2 | TAU_ITR151 | 2201 |
| 22 | CAG | IPN005 | LH | (G4S)3 | TAU_ITR152 | 2202 |
| 23 | CAG | IPN006 | LH | (G4S)4 | TAU_ITR153 | 2203 |
| 24 | CAG | IPN007 | LH | (G4S)5 | TAU_ITR154 | 2204 |
| 25 | CAG | IPN0014 | LH | (G4S)6 | TAU_ITR155 | 2205 |

Viral genome constructs for IPN002 scFv expression driven by a CAG promoter (TAU_ITR137 to TAU_ITR143; SEQ ID NO: 2187-2193 and TAU_ITR150 to TAU_ITR155; SEQ ID NO: 2200-2205, in the Table above) were tested in vitro. Western blot of 293 cell supernatant demonstrated bands (25-30 kd) for each construct tested, though the bands observed for TAU_ITR137 (SEQ ID NO: 2187), TAU_ITR138 (SEQ ID NO: 2188), TAU_ITR150 (SEQ ID NO: 2200) and TAU_ITR151 (SEQ ID NO: 2201) were light and difficult to resolve. At greater exposure, bands for these constructs became more evident. CAG-promoter scFv constructs were further tested by PHF and wild-type tau ELISA. Data are shown in Table 127 below.

TABLE 127
IPN002 scFv screening results

| | | | | wtTau | PHF Tau |
|---|---|---|---|---|---|
| Construct | | | SEQ | ELISA | ELISA |
| No. | Construct Description | Construct ID | ID NO: | EC₅₀ | EC₅₀ |
| 7 | CAG-IPN002-HL-G4S | TAU_ITR137 | 2187 | 3.0 | 1.2 |
| 8 | CAG-IPN002-HL-G4S2 | TAU_ITR138 | 2188 | 5.4 | 2.1 |
| 9 | CAG-IPN003-HL-G4S3 | TAU_ITR139 | 2189 | 2.5 | 1.1 |
| 10 | CAG-IPN008-HL-G4S4 | TAU_ITR140 | 2190 | 3.4 | 3.5 |
| 11 | CAG-IPN0010-HL-G4S5 | TAU_ITR141 | 2191 | 7.3 | 7.2 |
| 12 | CAG-IPN0011-HL-G4S6 | TAU_ITR142 | 2192 | 8.7 | 20.6 |
| 20 | CAG-IPN004-LH-G4S | TAU_ITR150 | 2200 | 4.9 | 6.0 |
| 21 | CAG-IPN004-LH-G4S2 | TAU_ITR151 | 2201 | 8.1 | 5.3 |
| 22 | CAG-IPN005-LH-G4S3 | TAU_ITR152 | 2202 | 4.6 | 8.0 |
| 23 | CAG-IPN006-LH-G4S4 | TAU_ITR153 | 2203 | 7.2 | 18.7 |
| 24 | CAG-IPN007-LH-G4S5 | TAU_ITR154 | 2204 | 7.0 | 10.0 |
| 25 | CAG-IPN0014-LH-G4S6 | TAU_ITR155 | 2205 | 5.2 | 11.3 |

A second set of ELISA assays were conducted to compare CAG-IPN0011-HL-G4S6 and CAG-IPN0012-HL-G4S8, and these data are shown below (Table 128).

TABLE 128
IPN002 scFv screening results

| | | | | wtTau | PHF Tau |
|---|---|---|---|---|---|
| Construct | | | SEQ | ELISA | ELISA |
| No. | Construct Description | Construct ID | ID NO: | EC₅₀ | EC₅₀ |
| 12 | CAG-IPN0011-HL-G4S6 | TAU_ITR142 | 2192 | 9.2 | 3.6 |
| 13 | CAG-IPN0012-HL-G4S8 | TAU_ITR143 | 2193 | 8.0 | 4.0 |

Data from these studies indicate that for scFv expression, the length of the linker between the antibody chains is important and effects the expression of the IPN002 scFv. Results of the ELISA assays indicate that the expressed IPN002 scFvs bind both wild-type and PHF tau.

### Viral Genomes for the Expression of PT3 Fragments

Twenty-four PT3 scFv constructs (TAU_ITR156 to TAU_ITR179: SEQ ID NO: 2206-2229) were made using one of three promoters (CAG, GFAP or synapsin), alternating PT3 antibody sequence configurations (heavy-linker-light (HL) vs light-linker-heavy (LH)) and flexible G4S linkers (“G4S” disclosed as SEQ ID NO: 4535) of different lengths. In three of these constructs, an alternate signal sequence (Glue100LP, hGhormone, pSec) was incorporated into the viral genome (TAU_ITR176 to TAU_ITR178; SEQ ID NO: 2226-2228), while in the others a heavy chain or light chain signal sequence was used. Each of the constructs included an HA tag, a rabbit globin poly(A) sequence, and 5′ and 3′ ITRs.

Expression of PT3 scFv fragments was tested in HEK293 cells followed by Western blot and binding affinity for ePHF tau of the expressed antibody was assessed by ELISA. As compared to TAU_ITR112 (SEQ ID NO: 2162; PT3-CAG-HT2AL), scFv fragments generally showed lower affinity to ePHF tau.

Eight viral genomes were designed for the expression of PT3 Fab fragments, using a CAG promoter, alternating PT3 antibody sequence configurations (heavy-linker-light (HL) vs light-linker-heavy (LH)), a T2A or F2A linker, rabbit globin poly(A) sequence, and 5′ and 3′ ITRs. As above, these constructs were tested for antibody expression in HEK293 cells followed by Western blot and for binding affinity for ePHF tau by ELISA. Each of the PT3 Fab constructs tested showed ability to bind ePHF tau.

### Viral Genomes for the Expression of C10.2 Fragments

Viral genomes for expression of C10.2 fragments (Fab, F(ab′)2, scFv) were generated and tested in vitro for expression and quantitation by Western blot/ELISA and for binding affinity to ePHF tau and concentration, also by ELISA.

First, four Fab and two F(ab′)2 constructs were designed using a CAG promoter and alternating antibody sequence configurations (heavy-linker light and light-linker-heavy). A T2A linker was used in each of the six viral genomes. Expression of C10.2 antibody fragments from these viral genomes was tested in vitro in 293 cells and the supernatant collected for western blot analysis. Concentration and binding affinity for tau of C10.2 F(ab′)2 and Fab was determined by ELISA.

Next, eight C10.2 scFv constructs were prepared using a CAG promoter, alternating C10.2 antibody sequence configurations (heavy-linker-light (HL) vs light-linker-heavy (LH)) and flexible G4S linkers (“G4S” disclosed as SEQ ID NO: 4535) of different lengths. Each of the constructs further included an HA tag, a poly(A) sequence, and 5′ and 3′ ITRs. Western blot analysis of 293 cell supernatant was used to test expression of C10.2 scFv and concentration and binding affinity for tau determined by ELISA.

One viral genome (CAG-C10.2 scFv HL-G4S3) was selected for further testing of 14 different signal peptides. Each of the fourteen signal peptides was cloned into a viral genome and the viral genomes tested for expression of C10.2 scFv in vitro.

Viral genomes for cell-specific expression of C10.2 Fab, F(ab′)2 and scFv fragments were generated using GFAP or synapsin promoters. Each of the 8 scFv constructs utilized the light-linker-heavy antibody sequence configuration with flexible G4S linkers (“G4S” disclosed as SEQ ID NO: 4535) of different lengths.

Taken together, ELISA assays indicate that C10.2 F(ab′)2 fragments bind pathological tau with an affinity similar to that seen with full C10.2 antibody, whereas C10.2 Fab fragments demonstrate a reduced affinity for pathological tau.

### Example 11. Identification of Antibody Epitopes

Anti-tau antibody PT3 is known to bind PHF-tau while not binding wild-type (441 amino acids: tau441; SEQ ID NO: 2127; NP_005901.2). Affinity for PHF-tau was measured at 18 pM, however, the specific epitope remains unknown, ePHF tau ELISA assay (EC₅₀ of 7.6) and SDS-PAGE showed that PT3 binds to ePHF tau.

Purified recombinant phospho-tau proteins were generated to simulate the mixture of abnormal hyperphosphorylated oligomeric tau that comprises ePHF tau and used in experiments to determine the antigen binding site for anti-tau antibody PT3. Recombinant phospho-tau proteins were phosphorylated in vitro by protein kinases Glycogen synthase kinase 3 beta (GSK3β), Protein Kinase A (PKA), Tau tubulin kinase 1 (TTBK1), Dual specificity tyrosine phosphorylation regulated kinase 1A (DYRK1A), BR serine/threonine-protein kinase 2 (BRSK2), Ca²⁺/Calmodulin-dependent protein kinase II (CAMK2), BR serine/threonine-protein kinase 1 (BRSK1), Phosphorylase kinase catalytic subunit gamma 2 (PHKG2). PHF tau was used as a control. Phospho-tau ELISA assay and Western blot analyses showed that PT3 binds recombinant phospho-tau. Results indicated that PT3 bound with greatest affinity to tau protein phosphorylated by DYRK1A, as compared to those phosphorylated by other kinases.

Protein kinase DYRK1A has serine/threonine/tyrosine protein kinase activity and has been shown to play a role in regulation of cell growth, division and differentiation. DYRK1A is important in the development of the nervous system and has been identified in formation and maturation of dendritic spines. DYRK1A has been shown to be over-expressed in Down Syndrome and has been associated with early onset of AD-type pathology, Down Syndrome and Autism Spectrum Disorders. Previous studies (Wegiel, J. et al., 2011, FEBS J 278(2):236-45, the contents of which are herein incorporated by reference in their entirety) indicate that DYRK1A phosphorylates human tau at 11 threonine (pThr) and serine (pSer) sites.

Tau441 was incubated in vitro with DYRK1A for 0, 5, 10, 20, 45, 82 or 120 minutes and then analyzed by western blot using PT3. Binding of PT3 to phospho-tau was increased with each increase in incubation time, with the greatest binding evident at 120 minutes.

Twelve recombinant tau mutant proteins were generated, based on phosphorylation sites of Tau441 by DYRK1A, having mutations at T181, S199, S202, T205, T212, T217, T231, S396, S400, S404 and S422. Amino acids at these positions were mutated to Alanine and a his tag was added as a tag or selectable marker.

Western blot of the 12 tau441 mutants and wild-type tau441 using anti-tau antibody PT3 showed no binding to recombinant tau proteins with mutations at T212 or T217, even though western blot using anti-his antibody showed recombinant tau441 protein present in each of these lanes. Semi-quantitative analyses of these western blots indicated substantially equivalent recombinant tau protein present based on anti-his staining. PT3 binding was more variable with greatest signal for recombinant tau 441 with a mutation at S422. Further quantification of PT3 signal normalized to anti-his signal indicated greatest binding of PT3 to recombinant tau441 with a mutation at S199 and then with decreasing binding for each of recombinant tau441 proteins with mutations at S422, T205, S400, S404, T181, S396, T231, wild-type tau441, S202, respectively.

ELISA was performed using PT3 as the immobilized antibody and DYRK1A phosphorylated tau proteins or recombinant tau mutant proteins in the applied solution (333 ng/mL). Data are shown in Table 129 below.

TABLE 129
PT3, Tau and Tau mutant ELISA

| Tau variant | OD450-570 |
|---|---|
| Phospho_Tau441 | 0.5 |
| Tau441 | 0.0 |
| Phospho_Tau441_T212A | 0.0 |
| Tau441_T212A | 0.0 |
| Phospho_Tau441_T202A | 0.6 |
| Tau441_T202A | 0.0 |
| Phospho_Tau441_S404A | 0.6 |
| Tau441_S404A | 0.0 |
| Phospho_Tau441_T181A | 0.4 |
| Tau441_T181A | 0.0 |
| Phospho_Tau441_S199A | 0.7 |
| Tau441_S199A | 0.0 |
| Phospho_Tau441_T205A | 0.4 |
| Tau441_T205A | 0.0 |
| Phospho_Tau441_T217A | 0.0 |
| Tau441_T217A | 0.0 |
| Phospho_Tau441_T231A | 0.3 |
| Tau441_T231A | 0.0 |
| Phospho_Tau441_S396A | 0.5 |
| Tau441_S396A | 0.0 |
| Phospho_Tau441_S400A | 0.3 |
| Tau441_S400A | 0.0 |
| Phospho_Tau441_S422A | 0.3 |
| Tau441_S422A | 0.1 |

As by western blot analysis, results again indicated poor binding of anti-tau antibody PT3 to tau with mutations at T212 or T217.

Six additional recombinant tau mutant proteins were generated to further elucidate the PT3 epitope (tau441−S214A, tau441−T212A+S214A, tau441−T212A+T217A, tau441−T217A+T231A, tau441−T212A+T231A, tau441−T212A+T217A+T231A). Western blot of these six additional tau441 mutants and wild type tau441 using PT3 showed no binding to recombinant tau proteins with mutations at T212 or T217, even though western blot using anti-his antibody showed recombinant tau441 protein present in each of these lanes.

PT3 ELISA using 210 ng/mL of phospho-Tau variants gave results as shown in the Table below (see Table 130) and confirmed the findings of the western blot analyses, wherein binding of PT3 was eliminated with recombinant tau proteins with mutations at T212 or T217.

TABLE 130
PT3 and phosphoTau ELISA

| Tau variant | OD450-570 |
|---|---|
| Wild-type Tau441 | 1.7 |
| Tau441-T212A | 0.0 |
| Tau441-S214A | 1.5 |
| Tau441-T217A | 0.0 |
| Tau441-T231A | 2.0 |
| Tau441-T212A + S214A | 0.1 |
| Tau441-T212A + T217A | 0.0 |
| Tau441-T217A + T231A | 0.1 |
| Tau441-T212A + T231A | 0.1 |
| Tau441-T212A + T217A + T231A | 0.0 |
| Tau441_no phos | 0.0 |
| Tau441-T212A-no phos | 0.0 |

A series of nine new phospho-tau peptides were synthesized as indicated in Table 131 below, where “p” indicates a phosphorylation site and tested by PT3 ELISA.

TABLE 131
Phospho Tau Peptides

| | | | SEQ | |
|---|---|---|---|---|
| Tau | | | ID | |
| ID | Description | Sequence | NO: | EC₅₀ |
| Tau1 | pT212 | SPGTPGSRSRpTPSLP | 2128 | — |
| | | TPP | | |
| Tau2 | pT217 | SRTPSLPpTPPTREPK | 2129 | 17.8 |
| Tau3 | pT231 | KKVAVVRpTPPKSPSS | 2130 | — |
| Tau4 | pT212 + | TPGSRSRpTPSLPpTP | 2131 | 13.6 |
| | pT217 | PTREPK | | |
| Tau5 | pT217 + | SRTPSLPpTPPTREPK | 2132 | 15.6 |
| | pT231 | KVAVVRpTPPKSPSS | | |
| Tau6 | pT212 + | TPGSRSRpTPSLPpTP | 2133 | 12.0 |
| | pT217 + | PTREPKKVAVVRpTPP | | |
| | pT231 | KSPSS | | |
| Tau7 | wt tau | TPGSRSRTPSLPTPPT | 2134 | — |
| | | REPKKVAVVRTPPKSP | | |
| | | SS | | |
| Tau8 | pT212 + | TPGSRSRpTPSLPTPP | 2135 | — |
| | pT231 | TREPKKVAVVRpTPPK | | |
| | | SPSS | | |
| Tau9 | wt tau | TPPSSGEPPKSGDRSG | 2136 | — |
| | | YSSPGSPGTPGSRSRT | | |
| | | PSLPTPPTREPKKVAV | | |
| | | V | | |

Further analysis of the Tau1 peptide by PT3 or AT100 ELISA or ELISA using a series of pT212 commercial antibodies confirmed that the Tau1 peptide was indeed phosphorylated at pT212.

Together, the data above indicate that PT3 binds tau phosphorylated at two sites, pT212 and pT217. A follow-up PT3 ELISA using peptides Tau1 (pT212), Tau2 (pT217), Tau4 (pT212+pT217) and Tau7 (wt tau) yielded EC₅₀ values of 83.6 and 17.2 for Tau2 (pT217) and Tau4 (pT212+pT217) peptides respectively. A PT3 competition ELISA using Tau1-6, Tau9 and DYRK1A phosphorylated Tau441 supported these findings. Based on these findings, a hypothetical model of PT3 binding was developed, wherein PT3 binds pT212 tau very weakly and pT217 with greater affinity (approximately 1,000× stronger). Tau phosphorylated at both pT212 and pT217 shows the greatest binding by PT3, with an affinity higher than that of PT3 to either phospho-site on its own. It was noted that PT3 binds wild type tau441 weakly as well.

### Example 12. Engineering of Antibody Variants

### Intrabodies

Viral genomes for expressing anti-tau intrabodies are engineered to test the effect of intracellular antibody expression. Secretory signal peptides are removed to retain the intrabody within the cell. Constructs are tested with the assays (ELISA and Western blot) described above. Intrabodies are not expected to be present in the cell supernatant, but are expected to be abundant in the cell lysate.

### Nanobodies

Viral genomes for expressing anti-tau nanobodies are developed and tested using the assays described above. Nanobodies may demonstrate better permeability in tissues, greater stability, higher solubility, shorter plasma half-life, and enhanced binding to hidden targets.

### Bispecific Antibodies

Viral genomes for expressing bispecific antibodies, bispecific nanobodies or bispecific scFvs are engineered and tested using the assays described above or any appropriate qualitative or quantitative assay known to one with skill in the art.

### Example 13. Intramuscular Expression of Antibodies

### Engineering for Enhanced Muscle Tropism

AAV particles comprising viral genomes encoding anti-tau antibodies are modified for enhanced transduction to muscular tissue. The AAV capsid is engineered to increase tropism to muscle. In one embodiment, AAV9 is modified to increase muscle tropism. In another embodiment, AAV2 and/or AAV3 are modified to increase muscle tropism. AAV particles may be delivered directly to muscle via intramuscular delivery or may be administered systemically, but still target muscular tissues.

### In Vivo Intramuscular Dosing Studies for Expression of Anti-Tau Antibodies

AAV1, AAV2 or VOY101 AAV particles comprising viral genomes encoding an anti-tau antibody (e.g., IPN002, PT3, C10.2, PHF1, MC1, CP13) are provided to subject (e.g., mouse, rat, rabbit, guinea pig, pig, ferret, dog, cat, non-human primate, or human) in a single intramuscular dose or in three intramuscular doses (1e12, 3e11, or 1e11) to the gastrocnemius muscle at a volume of 30-50 μL/site. The AAV particles will be formulated in PBS with 0.001% F-68. The subject may be wild-type, diseased, or model a disease or indication. Serum, CSF and/or tissue samples (hippocampus, cortex, thalamus, brain stem, spinal cord, liver, skeletal muscle) are collected for quantification of anti-tau antibody levels, with repeated sample collection for monitoring antibody presence over time. Serum will be collected at −2, 7, 14, 21, 28, 35, 42 and 43 days, as counted from day of administration of AAV particles, passive immunization or control composition. Antibody levels will be quantified using Tau-ELISA or ePHF ELISA, as described above. It is expected that administration of AAV particles, especially at the highest dose, will lead to detectable IPN002 antibody expression in serum samples. Vector genome quantification will be determined for a subset of tissues, such as for muscle, brain, and/or peripheral tissue (e.g., liver). Passive immunization with an anti-tau antibody (60 mg/kg) will be used as a control. Study designs are shown in Tables 132 and 133 below.

TABLE 132
AAV1.IPN002 IM study design

| | | SEQ | | | | | | End |
|---|---|---|---|---|---|---|---|---|
| Construct | TAU_ITR | ID | | Dose | Dose | | | of |
| Configuration | ID | NO: | μL | (vg/kg) | (vg) | Route | N | study |
| CAG-IPN002- | TAU_ITR1 | 1990 | 4 × 40 | 5E13 | 1E12 | IM | 6 | D 43 |
| HF.T2AL | | | | | | | | |
| CAG-IPN002- | TAU_ITR1 | 1990 | 2 × 40 | 1.5E13 | 3E11 | IM | 6 | D 43 |
| HF.T2AL | | | | | | | | |
| CAG-IPN002- | TAU_ITR1 | 1990 | 1 × 40 | 5E12 | 1E11 | IM | 6 | D 43 |
| HF.T2AL | | | | | | | | |
| Passive | — | — | | 60 | — | i.p. | 6 | D 43 |
| immunization | | | | mg/kg | | | | |
| with IPN002 | | | | weekly | | | | |
| PBS with | — | — | 100 | — | — | IM | 4 | D 43 |
| 0.001% F-68 | | | | | | | | |

TABLE 133
AAV2.IPN002 IM study design

| | | SEQ | | | | | | End |
|---|---|---|---|---|---|---|---|---|
| Construct | TAU_ITR | ID | | Dose | Dose | | | of |
| Configuration | ID | NO: | μL | (vg/kg) | (vg) | Route | N | study |
| CAG-IPN002-HF.T2AL | TAU_ITR1 | 1990 | 4 × 40 | 4E13 | 8E11 | IM | 6 | D 43 |
| CAG-IPN002-HF.T2AL | TAU_ITR1 | 1990 | 2 × 40 | 1.5E13 | 3E11 | IM | 6 | D 43 |
| CAG-IPN002-HF.T2AL | TAU_ITR1 | 1990 | 1 × 40 | 5E12 | 1E11 | IM | 6 | D 43 |

Secondary readouts include cage-side observations for the first week and weekly body weight checks. No adverse side effects are anticipated.

### Example 14. Intramuscular Dosing of Anti-Tau Antibody AAV Particles

To assess anti-tau antibody levels in serum, CSF or CNS tissue of mice after bilateral intramuscular delivery of vector, a viral genome (TAU_ITR112; SEQ ID NO: 2162; PT3-CAG-HT2AL) was packaged into AAV1, AAV2 or VOY101 capsids. AAV particles were formulated in PBS with 0.001% F-68 and provided at a dose of 4.65×10¹³ vg/kg across four intramuscular injection sites in the gastrocnemius and thigh muscle of C57Bl/6 mice. Forty-two days after administration, mice were sacrificed by intracardiac perfusion and CSF and/or tissue samples (hippocampus, cortex, thalamus, brain stem, spinal cord, olfactory bulb, liver, skeletal muscle) were collected for analysis. Serum samples were collected at −2, 7, 14, 21, 28, 35, 42 and 43 days, as counted from day of administration of AAV particles, passive immunization or vehicle control (PBS and 0.001% F-68). Passive immunization with an anti-tau antibody (30 mg/kg) was used as a control. Study design is shown in Table 134 below.

TABLE 134
PT3 IM study design

| | | SEQ | | | | | | End |
|---|---|---|---|---|---|---|---|---|
| | TAU_ITR | ID | | Dose | Dose | | | of |
| AAV Particle | ID | NO: | μL | (vg/kg) | (vg) | Route | N | study |
| AAV1.CAG-PT3-HF.T2A L | TAU_ITR112 | 2162 | 4 × 40 | 4.65E13 | 1E12 | IM | 8 | D 43 |
| AAV2.CAG-PT3-HF.T2AL | TAU_ITR112 | 2162 | 4 × 40 | 4.65E13 | 1E12 | IM | 8 | D 43 |
| VOY101.CAG-PT3-HF.T2AL | TAU_ITR112 | 2162 | 4 × 40 | 4.65E13 | 1E11 | IM | 8 | D 43 |
| Passive immunization with | — | — | | 30 | — | i.p. | 8 | D 43 |
| PT3 | | | | mg/kg | | | | |
| | | | | weekly | | | | |
| PBS with 0.001% F-68 | — | — | 100 | — | — | IM | 4 | D 43 |

Vector genome quantification was determined for a subset of tissues, such as for muscle (injection/non-injection sites), brain, and/or peripheral tissue (e.g., liver) by droplet digital PCR and normalized per diploid cell (VG/DC). Antibody levels in the hippocampus, cortex, spinal cord, liver, serum, gastrocnemius and CSF were quantified using ePHF ELISA, as described above. Secondary readouts included cage-side observations for one week after AAV particle administration and weekly body weight measurements. Vector genome biodistribution quantifications (VG/DC) are shown in Table 135 below.

TABLE 135
Vector genome quantification of ssVOY101.PT3 constructs

| | | | | Passive | Vehicle |
|---|---|---|---|---|---|
| | AAV1.PT3 | AAV2.PT3 | VOY101.PT3 | Immunization | (PBS) |
| Hippocampus | 0.1 | 0.3 | 8.6 | 0.0 | 0.0 |
| Cortex | 0.1 | 0.0 | 14.8 | 0.0 | 0.0 |
| Olfactory Bulb | 0.0 | 0.0 | 11.5 | 0.0 | 0.0 |
| Thalamus | 0.2 | 0.0 | 21.7 | 0.0 | 0.0 |
| Brainstem | 0.2 | 0.0 | 19.1 | 0.0 | 0.0 |
| Spinal Cord | 0.3 | 0.0 | 10.9 | 0.0 | 0.0 |
| Gastrocnemius | 61.0 | 27.5 | 6.7 | 0.0 | 0.0 |
| Left Bicep | 1.0 | 8.4 | 0.3 | 0.0 | 0.0 |
| Right Thigh | 71.3 | 8.9 | 4.2 | 0.0 | 0.0 |
| Spleen | 2.8 | 1.9 | 1.0 | 0.0 | 0.0 |
| Liver | 12.6 | 0.1 | 1.8 | 0.0 | 0.0 |

Subsequent to bilateral intramuscular delivery of TAU_ITR112 (SEQ ID NO: 2162; PT3-CAG-HT2AL) in AAV1, AAV2 or VOY101 very low biodistribution in CNS tissues was observed with AAV1 and AAV2 vectors. VOY101.PT3 showed higher viral genome levels per diploid cell across all tested CNS tissues.

In peripheral tissues, AAV1, AAV2 and VOY101 delivery of PT3 resulted in detectable biodistribution at the site of the injection (gastrocnemius and thigh skeletal muscle), but presence of viral genomes in distant tissues from the injection site (e.g., left bicep), was scare. In skeletal muscle, AAV1 particles showed a trend for increased biodistribution as compared to AAV2 and VOY101, after IM administration. Viral genome quantifications indicate that, in gastrocnemius and thigh tissue, AAV1 was the most efficient at delivering PT3 encoding viral genomes, while VOY101 was least efficient in these peripheral tissues.

ePHF ELISA was used to assess PT3 antibody expression in tissues collected after IM injection of AAV1, AAV2, or VOY101 particles. Samples of hippocampus, cortex, spinal cord, serum, liver and gastrocnemius were analyzed. Data are shown in Table 136 below as ng/mg protein.

TABLE 136
PT3 expression from AAV1, AAV2, or VOY101 particles (ng/mg protein)

| | | | | Passive | Vehicle |
|---|---|---|---|---|---|
| | AAV1.PT3 | AAV2.PT3 | VOY101.PT3 | Immunization | (PBS) |
| | ng/mg | ng/mg | ng/mg | ng/mg | ng/mg |
| Hippocampus | 3.9 | 0.0 | 47.3 | 0.0 | 0.0 |
| Cortex | 1.6 | 0.0 | 104.9 | 0.0 | 0.0 |
| Spinal Cord | 0.0 | 0.0 | 27.6 | 0.5 | 0.0 |
| Liver | 165.0 | 34.6 | 56.8 | 24.3 | 0.0 |
| Gastrocnemius | 1233.0 | 353.0 | 563.7 | 58.0 | 0.0 |
| Serum (μg/mL) | 1900.0 | 247.5 | 689.0 | 315.0 | 0.0 |

Based on PT3 antibody expression quantification as determined by ePHF ELISA, VOY101 was the only one of the three capsids to deliver detectable levels of PT3 to tissues of the CNS after IM dosing. Passive immunization and administration of vehicle resulted in substantially no detectable PT3 expression in tissue collected from the hippocampus, cortex and spinal cord. In peripheral tissues, PT3 antibody expression was detectable in liver and injected muscle (gastrocnemius) in all groups, other than vehicle, with the highest expression seen with delivery using AAV1, followed by VOY101 and AAV2, respectively. These results are consistent with the vector genome quantification (VG/DC) determined by ddPCR, above.

In serum collected on the terminal day, quantification of PT3 antibody expression demonstrated enhanced expression of PT3 when delivered with AAV1, as compared to AAV2, VOY101 and passive immunization. Whilst not wishing to be bound by theory, one might have expected approximately 200-400 μg/mL of antibody to be present in the serum of mice dosed with 30 mg/kg. In serum samples collected prior to the terminal day (at days 7, 14, 21, 28, 35, and 42 days), an abundance of PT3 antibody was detected in each of the samples and these levels were largely sustained throughout the entirety of the experiment. PT3 antibody levels in serum, delivered by AAV1 and VOY101 were higher than antibody levels evident after passive immunization, while AAV2 delivery of PT3 encoding viral genomes resulted in PT3 antibody levels in serum similar to those seen with passive immunization.

Cerebrospinal fluid samples collected on the terminal day were also assessed for PT3 expression after IM delivery with one of AAV1, AAV2, VOY101 or passive immunization. When delivered by passive immunization, PT3 antibody levels in the CSF were approximately 0.05% of the levels seen in serum samples. Whilst not wishing to be bound by theory, this finding is consistent with what might be expected by one with skill in the art. Data for PT3 antibody expression in CSF and serum samples is shown in Table 137 below.

TABLE 137
PT3 expression from AAV1, AAV2, or VOY101 particles (ng/mg protein)

| | | | | Passive | Vehicle |
|---|---|---|---|---|---|
| | AAV1.PT3 | AAV2.PT3 | VOY101.PT3 | Immunization | (PBS) |
| | ng/mg | ng/mg | ng/mg | ng/mg | ng/mg |
| Serum (μg/mL) | 2471.0 | 229.0 | 689.0 | 342.0 | 0.0 |
| CSF (ng/mL) | 851.0 | 174.8 | 886.0 | 157.5 | 0.0 |
| % CSF/Serum | 0.0 | 0.1 | 0.1 | 0.0 | 0.0 |

PT3 antibody expression in CSF was highest when delivered by AAV1 or VOY101, followed by AAV2 and passive immunization. Vectored delivery, regardless of capsid, resulted in greater CSF PT3 antibody expression than seen with passive immunization.

Taken together, these data indicate that intramuscular delivery of VOY101 can result in antibody expression in CNS tissues, while use of AAV1 leads to higher expression of antibody in peripheral tissues.

### Example 15. Evaluation of Anti-Tau Antibody Constructs in Non-Human Primates

Adult Rhesus macaque monkeys, pre-screened for low anti-AAV antibody levels, will receive intraparenchymal (IPa; e.g., thalamus and/or putamen), intracisternal (CM), intravenous (IV) or intramuscular (IM) administration of anti-tau antibody AAV particles to assess expression, distribution and therapeutic potential.

Anti-tau antibody AAV particles will be formulated in a solution comprising 180 mM sodium chloride, 10 mM sodium phosphate, and 0.001% Pluronic Acid. Dosing concentrations will be determined based on route of administration. For IPa administration, two animals will each receive bilateral infusions into two brain regions (e.g., the thalamus and putamen) by convection enhanced delivery device guided by MRI. An additional three animals will each receive a single 1 mL bolus injection into the CSF via the cisterna magna. Intravenous administration will be delivered via injection to the saphenous vein, or other readily accessible vein of the superior leg. Intramuscular injections will be administered in at least one, but up to four sites, of the same or of different muscles, such as the gastrocnemius muscle. One group of animals will receive passive immunization with the antibody constructs of interest. Animals will be monitored post-injection(s) for 28 days, with weekly body weight measurements and daily cage-side behavioral, mortality and morbidity checks serving as secondary readouts. Serum and CSF samples will be collected pre-dose and prior to necropsy.

On day 29, animals will be transcardially perfused with PBS, tissues will be collected and drop fixed in paraformaldehyde for histological analyses or flash frozen for biochemical assay. Tissues processed for histological analysis will be sectioned and immunostained with HRP-labeled mouse IgG1 for presence of tau antibodies. Further, these samples will be co-immunostained with NeuN, Iba1 or GFAP to identify cell-type. Samples snap frozen for biochemical analyses will be utilized for PCR to detect vector genomes and mRNA, ELISA to detect antibodies and MS to determine protein levels. Blood and CSF samples will be assessed for antibody and AAV levels.

### Example 16. Treatment of Tau-Associated Disease

AAV particles comprising viral genomes for delivery of an anti-tau antibody or fragment thereof are administered to a patient who has been diagnosed with a tau associated disease, disorder or condition. The purpose of the treatment may be aimed to manage the disease, prevent or slow the progression of the disease, treat the symptoms associated with the disease and/or cure the disease.

The AAV particles are administered to a subject by IM, IV, ICV, IPa or IT administration. The administration may include one or more injections over a period of time. The level and distribution of AAV particles and antibody expression is monitored by standard diagnostic techniques known in the art. Such diagnostic techniques include e.g. (e.g. from blood, urine, or saliva), cerebrospinal fluid (CSF) testing, or any other testing useful for monitoring antibody levels in the body.

Additionally, the progression of the disease and the health of the patient is monitored by standard diagnostic techniques known in the art. Such techniques may include diagnostic imaging (e.g. X-ray, MRI scans, Ultrasound scans, PET scans, Nuclear scans, mammography), biopsy, laboratory tests (e.g. from blood, urine, or saliva), cerebrospinal fluid (CSF) testing, vital signs, clinical tests (cognitive, motor or reflex tests) and other relevant techniques. Treatment with the AAV particles may result in cure of the tau-associated disease, slowing down or stabilizing the progression of the disease, or have no effect on the progression of the disease. Additionally, the treatment may reduce severity of one or more symptoms associated with the disease, eliminate one or more symptoms associated with the disease or have no effect on the symptoms.

### Example 17. Affinity of a Panel of Anti-Tau Antibodies to ePHF or Wild-Type Tau

A panel of eight BioLegend (San Diego, Calif.) monoclonal murine anti-tau antibodies were tested for their affinity to ePHF tau and wild-type tau (Tau441), using an ELISA assay as described in Example 4 (Tau441 immobilized on the 96-well plate for the wild-type tau assay, instead of ePHF tau). The panel of BioLegend antibodies included clones Tau5 (anti-tau, 210-230; cat #806401), Tau12 (anti-tau, 6-18; cat #806501), Tau46 (anti-tau, 404-421; cat #806601), 43D (anti-tau, 1-100; cat #816601), 2G9.F10 (anti-tau, 157-168; cat #824601), PHF-6 (anti-tau phospho Th231; cat #828901), PHF-13 (anti-tau phospho Ser396; cat #829001) and Tau-13 (anti-tau, 20-35; cat #835201), with PHF-1 used as a control antibody.

As described above, the ePHF were first immobilized on a 96-well plate overnight by pre-coating with 1500× of the concentrated PHF tau at 4° C., washed 3 times with PBS then blocked with 3% BSA for 2 hrs at room temperature or overnight at 4° C. Anti-tau antibodies were diluted in 3% BSA and loaded onto the plates. Plates were then incubated for 2 hrs at room temperature. Wells were washed 5 times with TBS/0.5% Tween 20 wash buffer, then incubated with 1:5000 dilution of anti-mouse antibody labeled with HRP (Thermo Fisher Scientific, Waltham, Mass.) for 30 min. Plates were then developed by incubating with one-step TMB substrate (Thermo Fisher Scientific, Waltham. Mass.) for 30 min. stopped by 2N H₂SO₄ and read using a BioTek Synergy H1 hybrid reader (BioTek, Winooski, Vt.) at 450 nm. The concentration of anti-tau antibodies, and their affinity for ePHF tau, was determined using a standard curve. The data are shown in Table 138 below.

TABLE 138
BioLegend antibody PHF Tan ELISA (OD450 data)

| ng/ML | Tau5 | | Tau12 | | Tau46 | | 43D | | 2G9.F10 | | PHF-6 | | PHF-13 | | Tau-13 | | PHF-1 | |
|---|---|---|---|---|---|---|---|---|---|---|---|---|---|---|---|---|---|---|
| 1000.0 | 3.2 | 3.2 | 3.2 | 3.1 | 2.8 | 2.9 | 3.2 | 3.1 | 3.1 | 3.1 | 3.2 | 3.1 | 1.9 | 2.0 | 3.2 | 3.2 | 2.1 | 2.2 |
| 333.3 | 3.2 | 3.2 | 3.2 | 3.1 | 2.3 | 2.6 | 3.2 | 3.1 | 2.9 | 3.0 | 3.0 | 3.1 | 1.2 | 1.2 | 3.2 | 3.2 | 1.5 | 1.6 |
| 111.1 | 3.2 | 3.2 | 3.2 | 3.2 | 1.7 | 2.0 | 3.2 | 3.2 | 2.4 | 2.8 | 2.7 | 2.9 | 0.6 | 0.7 | 3.2 | 3.2 | 1.2 | 1.3 |
| 37.0 | 2.6 | 2.9 | 3.2 | 3.2 | 0.9 | 1.0 | 3.2 | 3.2 | 1.6 | 2.0 | 1.9 | 2.3 | 0.3 | 0.3 | 3.2 | 3.2 | 0.8 | 0.9 |
| 12.3 | 1.7 | 2.1 | 3.2 | 3.2 | 0.4 | 0.4 | 3.0 | 3.1 | 0.7 | 0.9 | 1.1 | 1.3 | 0.1 | 0.1 | 2.8 | 3.1 | 0.5 | 0.5 |
| 4.1 | 0.8 | 0.9 | 2.2 | 2.4 | 0.2 | 0.1 | 1.7 | 1.9 | 0.3 | 0.3 | 0.4 | 0.5 | 0.1 | 0.0 | 1.4 | 1.8 | 0.2 | 0.2 |
| 1.4 | 0.4 | 0.4 | 1.0 | 1.0 | 0.1 | 0.0 | 0.7 | 0.8 | 0.1 | 0.1 | 0.2 | 0.2 | 0.0 | 0.0 | 0.6 | 0.7 | 0.1 | 0.1 |
| 0.5 | 0.1 | 0.1 | 0.4 | 0.4 | 0.0 | 0.0 | 0.3 | 0.3 | 0.0 | 0.0 | 0.1 | 0.0 | 0.0 | 0.0 | 0.2 | 0.2 | 0.1 | 0.1 |

PHF-13 showed the lowest affinity for ePHF tau, while all other BioLegend antibodies demonstrated greater affinity for ePHF tau than control antibody PHF-1. EC₅₀ values are shown in Table 139 below.

TABLE 139
EC₅₀ values for anti-tau antibodies

| Antibody | EC₅₀ |
|---|---|
| Tau5 | 9.3 |
| Tau12 | 2.2 |
| Tau46 | 79 |
| 43D | 3.3 |
| 2G9.F10 | 29.2 |
| PHF-6 | 20 |
| PHF-13 | 36542 |
| Tau-13 | 4.1 |
| PHF-1 | 1888 |

The same panel of antibodies was then tested for affinity to wild-type tau (Tau441) using an ELISA assay in which Tau441 was immobilized to the plate instead of ePHF tau as described above. All other aspects of the assay remained the same. OD450 and EC₅₀ data are shown in Tables 140 and 141 below. Phospho-tau specific antibodies PHF-6, PHF-13 and PHF-1 showed little to no affinity to Tau441, as expected.

TABLE 140
BioLegend antibody Tan 441 ELISA (OD450 data)

| Ng/ML | Tau5 | Tau12 | Tau46 | 43D | 2G9.F10 | PHF-6 | PHF-13 | Tau-13 | PHF-1 |
|---|---|---|---|---|---|---|---|---|---|
| 1000.0 | 3.3 | 3.3 | 3.2 | 3.2 | 2.2 | 0.1 | 0.1 | 3.2 | 0.1 |
| 333.3 | 3.3 | 3.3 | 3.2 | 3.3 | 1.6 | 0.0 | 0.1 | 3.2 | 0.1 |
| 111.1 | 3.3 | 3.3 | 3.0 | 3.2 | 0.9 | 0.0 | 0.1 | 3.1 | 0.1 |
| 37.0 | 2.6 | 3.1 | 2.0 | 3.0 | 0.5 | 0.0 | 0.0 | 2.3 | 0.1 |
| 12.3 | 1.2 | 2.0 | 1.0 | 2.0 | 0.2 | 0.0 | 0.1 | 1.2 | 0.1 |
| 4.1 | 0.6 | 0.9 | 0.4 | 0.9 | 0.1 | 0.0 | 0.0 | 0.5 | 0.1 |
| 1.4 | 0.2 | 0.4 | 0.2 | 0.4 | 0.1 | 0.1 | 0.0 | 0.2 | 0.1 |
| 0.0 | 0.1 | 0.0 | 0.1 | 0.0 | 0.0 | 0.0 | 0.0 | 0.0 | 0.1 |

TABLE 141
EC₅₀ values for anti-tau antibodies

| Antibody | EC₅₀ |
|---|---|
| Tau5 | 18.1 |
| Tau12 | 8.5 |
| Tau46 | 25.5 |
| 43D | 9.0 |
| 2G9.F10 | 235.3 |
| PHF-6 | 1.9 |
| PHF-13 | 481.8 |
| Tau-13 | 18.9 |
| PHF-1 | 1.4 |

### Example 18. Vectorized PT3 and IPN002 Expression in Primary Hippocampal Neurons

Primary neuronal cultures were used to test cell specificity, transduction efficiency and expression of PT3 & IPN002 using viral genomes listed in Table 142, encapsulated in Voy101. For preparation of primary cultures, brain tissue was removed from CDI mice at embryonic day 17 (E17) and meninges removed. The hippocampi and cortices were dissected out and digested with papain. Cells were counted and plated (17,500 cells/well) accordingly.

On day 4, in vitro (DIV4), cells were transduced with (3E6, 1E6, 3.33E5, 1.11E5, 3.7E4 VG/cell) AAV particles comprising the PT3 or IPN002 encoding viral genomes or VOY101-GFP as a positive control. Untransduced neurons served as the negative control.

Constructs shown in Table 142 with PT3 or IPN002 expression driven by ubiquitous or cell-type specific promoters were tested.

TABLE 142
PT3 & IPN002 Constructs for in vitro testing in primary cultures

| | SEQ | |
|---|---|---|
| Construct ID | ID NO: | Configuration |
| TAU_ITR104 | 2154 | CAG PT3 F2a HL |
| TAU_ITR108 | 2158 | CAG PTS F2a LH |
| TAU_ITR112 | 2162 | CAG PT3 T2A HL |
| TAU_ITR116 | 2166 | CAG PT3 T2a LH |
| TAU_ITR103 | 2153 | CBA PT3 F2a HL |
| TAU_ITR107 | 2157 | CBA PT3 F2a LH |
| TAU_ITR111 | 2161 | CBA PT3 T2A HL |
| TAU_ITR115 | 2165 | CBA PT3 T2a LH |
| TAU_ITR105 | 2155 | GFAP PT3 F2a HL |
| TAU_ITR109 | 2159 | GFAP PT3 F2A LH |
| TAU_ITR113 | 2163 | GFAP PT3 T2A HL |
| TAU_ITR117 | 2167 | GFAP PT3 T2A LH |
| TAU_ITR106 | 2156 | Syn PT3 F2a HL |
| TAU_ITR110 | 2160 | Syn PT3 F2a LH |
| TAU_ITR114 | 2164 | Syn PT3 T2A HL |
| TAU_ITR118 | 2168 | Syn PT3 T2a LH |
| TAU_ITR1 | 1990 | CAG.IPN002.HC.T2A.LC |
| TAU_ITR10 | 1999 | CAG.IPN002.LC.T2A.HC |
| TAU_ITR4 | 1993 | CBA.IPN002.HC.T2A.LC |
| TAU_ITR87 | 2137 | CBA.IPN002.LC.T2A.HC |
| TAU_ITR2 | 1991 | CAG.IPN002.HC.F2A.LC |
| TAU_ITR9 | 1998 | CAG.IPN002.LC.F2A.HC |
| TAU_ITR3 | 1992 | CBA.IPN002.HC.F2A.LC |
| TAU_ITR11 | 2000 | CBA.IPN002.LC.F2A.HC |

Cultures were confirmed to be approximately 60-70% neuronal as measured by NeuN staining. On DIV7, cells were fixed in 4% paraformaldehyde and 4% sucrose in PBS for 15 minutes followed by four PBS washes and used for immunocytochemical experiments. Cells were blocked with 3% BSA and 0.05% saponin in PBS for 1 hour at room temperature. Cells were stained with primary antibodies for IgG, GFAP, and NeuN and incubated overnight at 4 C in blocking buffer, then washed with four PBS washes. Cells were stained in secondary antibodies (1:1000 in blocking buffer) and incubated for 1 hour at room temperature followed by four washes in PBS. Höechst was applied at 1:1000 in PBS at room temperature for 15 minutes to stain nuclei, then washed four times with PBS. Imaging was performed using a BioTek Cytation 5. When transduced at 1E4 to 3E6 vg/cell, viral genomes comprising a GFAP-promoter, namely TAU_ITR109 (SEQ ID NO: 2159) TAU_ITR113 (SEQ ID NO:2163): TAU_ITR117 (SEQ ID NO: 2167) showed exclusive expression in GFAP positive astrocytes whereas viral genomes comprising a synapsin promoter, namely TAU_ITR106 (SEQ ID NO: 2156); TAU_ITR110 (SEQ ID NO: 2160); TAU_ITR114 (SEQ ID NO: 2164); TAU_ITR118 (SEQ ID NO: 2168), showed exclusive expression in NeuN positive neurons.

The percentage PT3 positive cells obtained by immunocytochemistry are shown in Table 143.

TABLE 143
% PT3 positive cells

| | Vg/cell | | |
|---|---|---|---|
| ITR ID | 3.33E+05 | 1.00E+06 | 3.00E+06 |
| TAU_ITR104 | 51.15 | 71.58 | 52.4 |
| | 56.31 | 69.06 | 57.72 |
| TAU_ITR108 | 60.94 | 73.51 | 81.1 |
| | 57.39 | 73.21 | 77.94 |
| TAU_ITR112 | 78.7 | 66.12 | 55.97 |
| | 62.31 | 57.62 | 59.5 |
| TAU_ITR116 | 30.28 | 52.03 | 71.06 |
| | 25.12 | 55.21 | 61.07 |
| TAU_ITR103 | 42.05 | 58.82 | 69.26 |
| | 53.65 | 64.46 | 68.38 |
| TAU_ITR107 | 31.7 | 51.4 | 82.18 |
| | 35.18 | 56.07 | 69.55 |
| TAU_ITR111 | 52.14 | 60.63 | 74.48 |
| | 56.23 | 59.93 | 51.03 |
| TAU_ITR115 | 22.54 | 45.92 | 57.17 |
| | 20 | 43.56 | 47.05 |

Among the CAG and CBA promoter driven constructs, higher percentage of PT3 positive cells was observed when the vg/cell concentration was increased. Some variations in the percentage PT3 positive cells was observed at the 3.33E+05 vg/cell concentration. But higher vg/cell concentrations showed similar % PT3 cells across the different constructs.

The percentage IPN002 positive cells obtained by immunocytochemistry are shown in Table 144.

TABLE 144
% IPN002 positive cells (ng/ml)

| | Vg/cell | | |
|---|---|---|---|
| ITR ID | 3.33E+05 | 1.00E+06 | 3.00E+06 |
| TAU_ITR1 | 25.17 | 38.95 | 56.4 |
| (CAG.IPN002.HC.T2A.LC) | 22 | 31 | 55.16 |
| TAU_ITR10 | 7.66 | 23.36 | 40.83 |
| (CAG.IPN002.LC.T2A.HC) | 7.33 | 20.96 | 35.51 |
| TAU_ITR2 | 1.31 | 9.72 | 21.45 |
| (CAG.IPN002.HC.F2A.LC) | 0.64 | 6.62 | 17.65 |
| TAU_ITR9 | 4.85 | 16.4 | 27.13 |
| (CAG.IPN002.LC.F2A.HC) | 3.34 | 13.42 | 26.57 |
| TAU_ITR4 | 10.82 | 23.65 | 38.77 |
| (CBA.IPN002.HC.T2A.LC) | 9.01 | 21.59 | 31.08 |
| TAU_ITR87 | 1.9 | 7.85 | 21.61 |
| (CBA.IPN002.LC.T2A.HC) | 1.38 | 6.34 | 15.31 |
| TAU_ITR3 | 3.2 | 7.94 | 20.58 |
| (CBA.IPN002.HC.F2A.LC) | 2.65 | 6.33 | 17.48 |
| TAU_ITR11 | 5.45 | 14.96 | 23.64 |
| (CBA.IPN002.LC.F2A.HC) | 3.49 | 10.49 | 20.4 |

Among the CAG and CBA promoter driven constructs, higher percentage of IPN002 positive cells was observed when the vg/cell concentration was increased. Some variations in the percentage PT3 positive cells was observed at the 3.33E+05 vg/cell concentration. In all three vg/cell concentrations tested TAU_ITR1 (CAG.IPN002.HC.T2A.LC) showed the highest percentage of IPN002 expression.

The supernatant collected on DIV7 (3 days after transduction) was used along with IgG1 mouse uncoated ELISA kit (ThermoFisher) for quantification of antibody/mL (ms IgG ng/mL). Comparison between the CAG promoter driven constructs (TAU_ITR112, TAU_ITR116, TAU_ITR104, TAU_ITR108) and CBA promoter driven constructs (TAU_ITR111, TAU_ITR115, TAU_ITR103, TAU_ITR107) comprising either a T2A cleavage site or F2A cleavage site showed that the CBA promoter provides more robust expression of PT3 when combined with T2A, as compared to CBA promoter driven PT3 expression combined with a T2A cleavage site. Further, viral genomes comprising a CAG or CBA promoter and a T2A cleavage site provided more robust PT3 expression than viral genomes comprising an F2A linker.

PT3 expression data are shown in Table 145.

TABLE 145
PT3 expression

| | Vg/cell | | |
|---|---|---|---|
| ITR ID | 3.33E+05 | 1.00E+06 | 3.00E+06 |
| TAU_ITR104 | 7.69 | 8.578 | 7.29 |
| | 7.657 | 8.289 | 6.681 |
| TAU_ITR108 | 10.03 | 11.05 | 12.58 |
| | 9.30 | 12.30 | 13.79 |
| TAU_ITR112 | 39.92 | 30.24 | 22.68 |
| | 37.07 | 29.00 | 21.74 |
| TAU_ITR116 | 17.16 | 28.18 | 34.44 |
| | 19.14 | 27.62 | 37.22 |
| TAU_ITR103 | 8.55 | 9.65 | 7.56 |
| | 7.18 | 11.58 | 9.23 |
| TAU_ITR107 | 6.32 | 9.02 | 9.06 |
| | 7.21 | 9.04 | 8.92 |
| TAU_ITR111 | 31.16 | 29.70 | 30.65 |
| | 34.09 | 39.55 | 38.47 |
| TAU_ITR115 | 14.70 | 20.02 | 21.02 |
| | 17.12 | 22.13 | 24.07 |

Among the constructs tested, TAU_ITR112 (CAG PT3 T2A HL), TAU_ITR111 (CBA PT3 T2A HL) with T2A.HL format showed comparatively higher PT3 expression levels than the other constructs tested. TAU_ITR115 with CBA PT3 T2A LH also showed high PT3 expression at the 1.00E+06 vg/cell concentration.

The PT3 expression (ng/ml) in primary hippocampal neurons when comprising a T2A cleavage site was higher than when an F2A cleavage site was used. A similar observation was made in vivo in the hippocampus and CSF of mice transduced with AAV particles comprising PT3 encoding viral genomes.

As a comparison, PT3 expression levels obtained by ELISA were compared to IPN002 antibody expression levels (ng/ml) and are shown in Table 146.

TABLE 146
IPN002 expression (ng/ml)

| | Vg/cell | | |
|---|---|---|---|
| ITR ID | 3.33E+05 | 1.00E+06 | 3.00E+06 |
| TAU_ITR1 | 25.17 | 38.95 | 56.4 |
| (CAG.IPN002.HC.T2A.LC) | 22 | 31 | 55.16 |
| TAU_ITR10 | 7.66 | 23.36 | 40.83 |
| (CAG.IPN002.LC.T2A.HC) | 7.33 | 20.96 | 35.51 |
| TAU_ITR2 | 1.31 | 9.72 | 21.45 |
| (CAG.IPN002.HC.F2A.LC) | 0.64 | 6.62 | 17.65 |
| TAU_ITR9 | 4.85 | 16.4 | 27.13 |
| (CAG.IPN002.LC.F2A.HC) | 3.34 | 13.42 | 26.57 |
| TAU_ITR4 | 10.82 | 23.65 | 38.77 |
| (CBA.IPN002.HC.T2A.LC) | 9.01 | 21.59 | 31.08 |
| TAU_ITR87 | 1.9 | 7.85 | 21.61 |
| (CBA.IPN002.LC.T2A.HC) | 1.38 | 6.34 | 15.31 |
| TAU_ITR3 | 3.2 | 7.94 | 20.58 |
| (CBA.IPN002.HC.F2A.LC) | 2.65 | 6.33 | 17.48 |
| TAU_ITR11 | 5.45 | 14.96 | 23.64 |
| (CBA.IPN002.LC.F2A.HC) | 3.49 | 10.49 | 20.4 |

In general, an increase in vg/cell concentrations resulted in higher IPN002 expression levels. The CAG promoter based constructs showed higher IPN002 expression when compared to CBA promoter driven expression.

The IPN002 expression (ng/ml) in primary hippocampal neurons when comprising a T2A cleavage site was higher than when an F2A cleavage site was used. A similar observation was made in vivo in the hippocampus and CSF of mice transduced with AAV particles comprising the IPN002 encoding viral genomes.

Taken together these data suggest that viral genomes comprising a T2A cleavage site may contribute to greater antibody expression than when an F2A cleavage site is used.

### Example 19. Expression and Distribution of scFv Based Anti-Tau Antibody Constructs

Testing of IPN002 scFv Viral Genomes In Vivo in Mice

To study anti-tau scFv antibody expression driven by CAG promoter in serum, CSF and/or CNS tissue of mice after intravenous delivery of AAV particles, viral genomes TAU_ITR139 (SEQ ID NO. 2189) and TAU_ITR152 (SEQ ID NO. 2202) were each packaged into VOY101 capsids. AAV particles were formulated in PBS and provided intravenously at a dose of 1.4×10¹³ vg/kg to 2 month-old female C57Bl/6 mice. 28 days after administration, mice were euthanized and CSF and/or tissue samples (hippocampus, cortex, thalamus, brain stem, spinal cord, olfactory bulb, liver, skeletal muscle) were collected for analysis. Study design is shown in Table 147.

TABLE 147
IPN002 IV study design

| | | SEQ | | | | | | End |
|---|---|---|---|---|---|---|---|---|
| | TAU_ITR | ID | | Dose | Dose | | | of |
| AAV Particle | ID | NO: | μl | (vg/kg) | (vg) | Route | N | study |
| IPN002.scFv.CAG.HL.G4S-3 | TAU_ITR139 | 2189 | 100 | 1.4 × 10¹³ | 2.8 × 10¹¹ | IV | 6 | Day 28 |
| IPN002.scFv.CAG.LH.G4S-3 | TAU_ITR152 | 2202 | 100 | 1.4 × 10¹³ | 2.8 × 10¹¹ | IV | 6 | Day 28 |
| PBS | — | — | 100 | — | — | IV | 4 | Day 28 |

Vector genome was quantified for a subset of tissues, such as for brain, CSF, and/or peripheral tissue (e.g. liver) by droplet digital PCR and normalized per diploid cell (VG/DC). Antibody levels in the hippocampus, cortex, olfactory bulb, brainstem, spinal cord, liver, serum, and CSF were quantified using ePHF ELISA, as described above. Vector genome biodistribution quantification (VG/DC) is shown in Table 148.

TABLE 148
Vector genome quantification of VOY101.IPN002
constructs (AAV genomes/cell)

| TAU_ITR ID | | | Olfactory | | | Spinal |
|---|---|---|---|---|---|---|
| (SEQ ID NO.) | Hippocampus | Cortex | bulb | Thalamus | Brainstem | Cord |
| TAU_ITR139 | 33.49 | 24.86 | 34.14 | 47.33 | 59.68 | 38.72 |
| (SEQ ID NO. | 37.06 | 23.72 | 63.95 | 58.46 | 71.12 | 141.68 |
| 2189) | 14.34 | 22.12 | 33.61 | 35.87 | 62.59 | 49.55 |
| | 11.32 | 19.52 | 55.37 | 33.25 | 48.86 | 36.27 |
| | 34 | 31.09 | 58.12 | 46.73 | 61.29 | 37.2 |
| | 20.28 | 33 | 50.18 | 48.68 | 83.83 | 106.23 |
| Average | 25.08 | 25.72 | 49.23 | 45.05 | 64.56 | 68.28 |
| TAU_ITR152 | 23.08 | 15.86 | 30.25 | 59.11 | 52.76 | 79.12 |
| (SEQ ID NO. | 2.78 | 3.38 | 10.85 | 6.84 | 9.77 | 12.14 |
| 2202) | 17.73 | 24.05 | 14.82 | 28.73 | 38.81 | 28.67 |
| | 32.01 | 25.15 | 43.66 | 73.5 | 79.95 | 116.56 |
| | 17.37 | 21.38 | 28.08 | 33.4 | 51.58 | 42.41 |
| | 3.46 | 8.82 | 7.33 | 11.04 | 16.31 | 10.79 |
| Average | 16.07 | 16.44 | 22.50 | 35.44 | 41.53 | 48.28 |
| Vehicle (PBS) | 0 | 0 | 0 | 0 | 0 | 0 |

Subsequent to intravenous delivery of IPN002-scFv VOY101 particles, biodistribution was observed in all CNS tissue noted in Table 148. Higher viral genome levels per diploid cell were observed with TAU_ITR139 (SEQ ID NO. 2189) with the IPN002.scFv.CAG.HL.G4S-3 (heavy-linker-light) configuration compared to TAU_ITR152 (SEQ ID NO. 2202) with the IPN002.scFv.CAG.LH.G4S-3 (light-linker-heavy) configuration.

ePHF ELISA was used to assess IPN002 antibody expression in tissues collected after intravenous injection of IPN002-scFv VOY101 particles. Samples of hippocampus, cortex, olfactory bulb, thalamus, brain stem and spinal cord were analyzed. Data are shown in Table 149 and Table 150 as ng/mg of protein and as normalized sFv/vg respectively.

TABLE 149
IPN002 expression in the CNS

| | Hippocampus | | Cortex | | Olfactory bulb | |
|---|---|---|---|---|---|---|
| TAU_ITR ID | ng/mg | | ng/mg | | ng/mg | |
| (SEQ ID NO.) | protein | scFv/Vg | protein | scFv/Vg | protein | scFv/Vg |
| TAU_ITR139 | 352.56 | 10.53 | 590.74 | 23.76 | 1096.45 | 32.12 |
| (SEQ ID NO. | 422.05 | 11.39 | 578.91 | 24.41 | 1147.95 | 17.95 |
| 2189) | 446.11 | 31.11 | 714.68 | 32.30 | 1011.01 | 30.08 |
| | 400.01 | 35.34 | 580.23 | 29.72 | 660.91 | 11.94 |
| | 448.99 | 13.20 | 629.62 | 20.25 | 1197.19 | 20.60 |
| | 505.11 | 24.90 | 757.7 | 22.96 | 1126.88 | 22.46 |
| Average | 429.14 | 21.08 | 641.98 | 25.57 | 1040.07 | 22.53 |
| TAU_ITR152 | 194.52 | 8.43 | 358.57 | 22.61 | 465.66 | 15.39 |
| (SEQ ID NO. | 24.19 | 8.69 | 33.18 | 9.81 | 44.32 | 4.08 |
| 2202) | 74.93 | 4.23 | 138.41 | 5.76 | 225.37 | 15.20 |
| | 233.75 | 7.30 | 454.64 | 18.08 | 599.41 | 13.73 |
| | 292.5 | 16.84 | 464.74 | 21.74 | 772.8 | 27.52 |
| | 57.63 | 16.66 | 95.61 | 10.84 | 190.22 | 25.93 |
| Average | 146.25 | 10.36 | 257.53 | 14.81 | 382.96 | 16.98 |
| Vehicle (PBS) | 0 | 0 | 0 | 0 | 0 | 0 |

TABLE 150
IPN002 expression in CNS and spinal cord

| TAU_ITR | Thalamus | | Brainstem | | Spinal Cord | |
|---|---|---|---|---|---|---|
| ID (SEQ ID | ng/mg | scFv/V | ng/mg | | ng/mg | |
| NO.) | protein | g | protein | scFv/Vg | protein | scFv/Vg |
| TAU_ITR139 | 639.27 | 13.51 | 639.49 | 10.71 | 883.76 | 22.82 |
| (SEQ ID NO. | 808.44 | 13.83 | 1296.27 | 18.23 | 836.37 | 5.90 |
| 2189) | 700.65 | 19.54 | 887.67 | 14.18 | 879.74 | 17.75 |
| | 763.33 | 22.96 | 1043.87 | 21.37 | 870.09 | 23.99 |
| | 786.91 | 16.84 | 675.48 | 11.02 | 986.58 | 26.52 |
| | 913.07 | 18.76 | 1128.73 | 13.46 | 744.17 | 7.01 |
| Average | 768.61 | 17.57 | 945.25 | 14.83 | 866.79 | 17.33 |
| TAU_ITR152 | 553.21 | 9.36 | 505.81 | 9.59 | 793.3 | 10.03 |
| (SEQ ID NO. | 54.94 | 8.04 | 78.31 | 8.01 | 99.97 | 8.23 |
| 2202) | 223.88 | 7.79 | 214.98 | 5.54 | 458.31 | 15.99 |
| | 522.09 | 7.10 | 656.5 | 8.21 | 900.14 | 7.72 |
| | 702.19 | 21.02 | 645.19 | 12.51 | 486.98 | 11.48 |
| | 161.88 | 14.66 | 266.84 | 16.35 | 202.79 | 18.79 |
| Average | 369.70 | 11.33 | 394.61 | 10.03 | 490.25 | 12.04 |
| Vehicle (PBS) | 0 | 0 | 0 | 0 | 0 | 0 |

High levels of IPN scFv were observed in the hippocampus, cortex and olfactory bulb of the mice dosed with IPN scFv vectors. Higher IPN scFv levels were observed with TAU_ITR139 (SEQ ID NO. 2189) with the IPN002.scFv.CAG.HL.G4S-3 viral genome configuration as compared to TAU_ITR152 (SEQ ID NO. 2202) with the IPN002.scFv.CAG.LH.G4S-3 viral genome configuration.

In serum and CSF samples collected on the terminal day, quantification of the IPN002 antibody expression demonstrated enhanced expression of IPN002 when delivered with VOY101 and driven by the CAG promoter. Consistent with other tissues, higher IPN scFv levels were observed with TAU_ITR139 (SEQ ID NO. 2189) with the IPN002.scFv.CAG.HL.G4S-3 configuration as compared to TAU_ITR152 (SEQ ID NO. 2202) with the IPN002.scFv.CAG.LH.G4S-3 configuration. These results are shown in Table 151.

TABLE 151
IPN002 expression in CSF and Serum

| TAU_ITR ID | CSF | | Serum | |
|---|---|---|---|---|
| (SEQ ID NO.) | ng/ml | nM scFv | ng/ml | nM scFv |
| TAU_ITR139 | 11926.1 | 477.04 | 54921 | 2196.84 |
| (SEQ ID NO. 2189) | 9711.8 | 388.47 | 119105 | 4764.20 |
| | 9993.2 | 399.73 | 59057 | 2362.28 |
| | 9846.95 | 393.88 | 28863.5 | 1154.54 |
| | 11596.75 | 463.87 | 59741.5 | 2389.66 |
| | — | — | 124490.5 | 4979.62 |
| Average | 10614.96 | 424.60 | 74363.08 | 2974.52 |
| TAU_ITR152 | 4702.4 | 188.10 | 9443.8 | 377.75 |
| (SEQ ID NO. 2202) | 1034.7 | 41.39 | 1083.95 | 43.36 |
| | 9470.5 | 378.82 | 844.85 | 33.79 |
| | 10020.6 | 400.82 | 3451.05 | 138.04 |
| | 3368.35 | 134.73 | 5346.7 | 213.87 |
| | — | — | 1351.75 | 54.07 |
| Average | 5719.31 | 228.77 | 3587.017 | 143.48 |
| Vehicle (PBS) | 0 | 0 | 0 | 0 |

Liver samples collected on the terminal day, showed low IPN002 antibody expression and biodistribution of AAV genomes when delivered with VOY101. The results are shown in Table 152.

TABLE 152
Vector genome quantification and IPN002 expression in Liver

| Construct | Genome/cell | scFv/mg |
|---|---|---|
| TAU_ITR139 | 7.50 | 222.83 |
| (SEQ ID NO. 2189) | 19.36 | 332.41 |
| | 9.18 | 289.83 |
| | 4.62 | 169.14 |
| | 7.79 | 256.53 |
| | 13.16 | 349.19 |
| Average | 10.27 | 269.99 |
| TAU_ITR152 | 7.99 | 127.28 |
| (SEQ ID NO. 2202) | 0.36 | 12.55 |
| | 0.22 | 9.41 |
| | 2.87 | 42.79 |
| | 3.13 | 90.54 |
| | 0.59 | 19.05 |
| Average | 2.53 | 50.27 |
| vehicle | 0 | 0 |

Consistent with other tissues, the liver demonstrated higher IPN scFv levels with delivery of TAU_ITR139 (SEQ ID NO. 2189) with the IPN002.scFv.CAG.HL.G4S-3 configuration as compared to with delivery of TAU_ITR152 (SEQ ID NO. 2202) with the IPN002.scFv.CAG.LH.G4S-3 configuration.

Brain tissue samples were collected on the terminal day and fixed with 4% PFA for immunohistochemistry. Anti-IgG immunohistochemistry of brain tissue collected from mice injected with VOY101.IPN002-scFv AAV particles driven by CAG promoters and one of viral genome configurations (HL or LH) showed broad antibody expression across mouse CNS, but stronger staining in tissue of mice treated with HL configuration as compared to the LH configuration. Vehicle-treated control animals exhibited low background levels of staining. Expression of IPN002 antibody was observed in a non-cell type specific manner (neurons and astrocytes showed staining) across multiple brain regions, as may be expected when using ubiquitous promoters. Expression of IPN002 was also observed in the spinal cord.

Taken together, these data indicate that the ubiquitous CAG promoter may effectively drive IPN002 expression in CNS tissue of mice, with limited concomitant expression in peripheral tissues, such as the liver.

The IPN002 scFv expression levels in the hippocampus and cortex were compared to expression levels of full-length PT3 antibody, IPN002 Fab fragment and IPN002 F(ab′)2 fragment. Both the heavy chain-linker-light chain format as well as the light chain-linker-heavy chain format was considered for this analysis. Comparison of the expression levels showed that the scFv format resulted in higher expression in both the hippocampus and the cortex as compared to the other formats (e.g., full length antibody, Fab or F(ab′)2 fragment). PT3 expression levels of the full-length antibody in the heavy-linker-light chain format was higher than IPN002 expression from Fab or F(ab′)2 fragments.

Testing of PT3 scFv Viral Genomes In Vivo in Mice

To study anti-tau antibody PT3 scFv levels in serum, CSF and/or CNS tissue of mice after intravenous delivery of vector, viral genomes TAU_ITR162 (SEQ ID NO: 2212). TAU_ITR168 (SEQ ID NO: 2218); TAU_ITR165 (SEQ ID NO: 2215) were packaged into VOY101 capsids. AAV particles were formulated in PBS and provided intravenously at a dose of 1.4×10¹³ vg/kg into 2 month-old female C57Bl/6 mice. Twenty-eight days after administration, mice were sacrificed and CSF and/or tissue samples (hippocampus, cortex, thalamus, brain stem, spinal cord, olfactory bulb, liver, spleen) were collected for analysis. Study design is shown in Table 153.

TABLE 153
PT3 IV study design

| | | SEQ | | | | | | End |
|---|---|---|---|---|---|---|---|---|
| | TAU_ITR | ID | | Dose | Dose | | | of |
| AAV Particle | ID | NO: | μL | (vg/kg) | (vg) | Route | N | study |
| ssAAV CAG PT3 VL G4S-3 | TAU_ITR162 | 2212 | 100 | 1.4 × 10¹³ | 2.8 × 10¹¹ | IV | 6 | Day 28 |
| VH HA rBGpA | | | | | | | | |
| ssAAV SYN PTS VL G4S-3 | TAU_ITR168 | 2218 | 100 | 1.4 × 10¹³ | 2.8 × 10¹¹ | IV | 6 | Day 28 |
| VH HA rBGpA | | | | | | | | |
| ssAAV GFAP PT3 VL G4S-3 | TAU_ITP165 | 2215 | 100 | 1.4 × 10¹³ | 2.8 × 10¹¹ | IV | 6 | Day 28 |
| VH HA rBGpA | | | | | | | | |
| PBS | — | — | 100 | — | — | IV | 4 | Day 28 |

Vector genome distribution was quantified for a subset of tissues, such as for brain, CSF, and/or peripheral tissue (e.g. liver and spleen) by droplet digital PCR and normalized per diploid cell (VG/DC). Antibody levels in the hippocampus, cortex, olfactory bulb, brainstem, spinal cord, liver, serum, and CSF were quantified using ePHF ELISA, as described above. Vector genome biodistribution quantification (VG/DC) is shown in Table 154.

TABLE 154
Biodistribution of PT3-scFv viral genomes (AAV genomes/cell)

| | | | Olfactory | | | Spinal |
|---|---|---|---|---|---|---|
| Construct | Hippocampus | Cortex | bulb | Thalamus | Brainstem | Cord |
| TAU_ITR168 | 10.40 | 8.63 | 12.60 | 32.13 | 20.31 | 9.53 |
| | 17.99 | 28.95 | 23.63 | 23.19 | 18.93 | 19.91 |
| | 9.20 | 6.32 | 20.24 | 21.44 | 23.49 | 31.58 |
| | 10.05 | 7.93 | 42.50 | 22.96 | 26.33 | 37.08 |
| | 13.67 | 8.67 | 12.63 | 29.11 | 33.83 | 20.46 |
| | 2.17 | 2.02 | 2.05 | 2.85 | 5.32 | 2.27 |
| | 11.58 | — | 18.11 | 23.05 | 45.04 | 66.61 |
| Average | 10.72 | 10.42 | 18.82 | 22.10 | 24.75 | 26.78 |
| TAU_ITR165 | 34.22 | 35.57 | 24.41 | 37.06 | 36.65 | 52.59 |
| | 5.89 | 13.29 | 6.17 | 9.49 | 9.98 | 23.06 |
| | 9.99 | 4.37 | 6.47 | 22.11 | 13.10 | 24.12 |
| | 32.62 | 46.88 | 63.20 | 79.94 | 80.27 | 84.26 |
| | 46.54 | 33.49 | 55.83 | 50.40 | 57.67 | 72.87 |
| | 33.88 | 15.20 | 62.29 | 74.33 | 72.13 | 121.54 |
| Average | 27.19 | 24.80 | 36.40 | 45.56 | 44.97 | 63.07 |
| TAU_ITR162 | 6.27 | 5.58 | 6.14 | 11.32 | 21.80 | 20.95 |
| | 13.76 | 13.39 | 14.91 | 21.34 | 44.88 | 57.40 |
| | 15.32 | 14.09 | 19.04 | 21.52 | 19.58 | 13.56 |
| | 14.26 | 12.43 | 26.25 | 61.08 | 49.97 | 142.85 |
| | 3.45 | 3.13 | 4.94 | 13.09 | 15.98 | 14.07 |
| | 9.22 | 9.15 | 41.04 | 17.63 | 38.43 | 22.21 |
| Average | 10.38 | 9.63 | 18.72 | 24.33 | 31.77 | 45.17 |
| Vehicle (PBS) | 0 | 0 | 0 | 0 | 0 | 0 |

Subsequent to intravenous delivery of PT3-scFv VOY101 particles, biodistribution was observed in all tissues noted in Table 154. Higher viral genome levels per diploid cell were observed in hippocampus, cortex, olfactory bulb and thalamus with TAU_ITR165 with the ssAAV GFAP PT3 VL G4S-3 VH HA rBGpA configuration compared to the other two constructs.

ePHF ELISA was used to assess PT3 antibody expression in tissues collected after intravenous injection of VOY101 particles. Samples of hippocampus, cortex, olfactory bulb, thalamus, brain stem and spinal cord were analyzed. Data are shown in Table 155 and Table 156 as ng/mg of protein and as normalized scFv/vg.

TABLE 155
PT3 expression in the CNS

| | Hippocampus | | Cortex | | Olfactory bulb | |
|---|---|---|---|---|---|---|
| | ng/mg | | ng/mg | | ng/mg | |
| Construct | protein | scFv/Vg | protein | scFv/Vg | protein | scFv/Vg |
| TAU_ITR168 | 0 | 0.00 | 3.63 | 0.42 | 3.69 | 0.29 |
| | 0 | 0.00 | 3.04 | 0.11 | 3.75 | 0.16 |
| | 0 | 0.00 | 2.50 | 0.40 | — | 0.00 |
| | 0.98 | 0.10 | 2.74 | 0.35 | 2.48 | 0.06 |
| | 0 | 0.00 | 3.66 | 0.42 | 8.17 | 0.65 |
| | 0 | 0.00 | 2.68 | 1.33 | — | 0.00 |
| | — | 0.00 | 2.64 | — | 1 | 0.06 |
| Average | 0.16 | 0.01 | 2.98 | 0.50 | 3.82 | 0.17 |
| TAU_ITR165 | 4.03 | 0.12 | 25.74 | 0.72 | 47.4 | 1.94 |
| | 0 | 0.00 | 2.95 | 0.22 | 1.14 | 0.18 |
| | 0 | 0.00 | 2.50 | 0.57 | 4.69 | 0.72 |
| | 2.26 | 0.07 | 16.53 | 0.35 | 39.03 | 0.62 |
| | 8.12 | 0.17 | 22.94 | 0.69 | 119.67 | 2.14 |
| | 0.99 | 0.03 | 5.36 | 0.35 | 28.34 | 0.45 |
| Average | 2.57 | 0.07 | 12.67 | 0.48 | 40.05 | 1.01 |
| TAU_ITR162 | 1.8 | 0.29 | 7.67 | 1.37 | 22.36 | 3.64 |
| | 5.35 | 0.39 | 15.81 | 1.18 | 53.01 | 3.56 |
| | 17.84 | 1.16 | 46.60 | 3.31 | 83.61 | 4.39 |
| | 18.15 | 1.27 | 67.42 | 5.42 | 146.79 | 5.59 |
| | 0.97 | 0.28 | 5.64 | 1.80 | 16.3 | 3.30 |
| | 8.47 | 0.92 | 53.74 | 5.87 | 119.46 | 2.91 |
| Average | 8.76 | 0.72 | 32.81 | 3.16 | 73.59 | 3.90 |
| Vehicle (PBS) | 0 | 0 | 0 | 0 | 0 | 0 |

TABLE 156
PT3 expression CNS and Spinal Cord

| | Thalamus | | Brainstem | | Spinal cord | |
|---|---|---|---|---|---|---|
| | ng/mg | | ng/mg | | ng/mg | |
| Construct | protein | scFv/Vg | protein | scFv/Vg | protein | scFv/Vg |
| TAU_ITR168 | 43.31 | 1.35 | 10.96 | 0.54 | 45.01 | 4.72 |
| | 6.8 | 0.29 | 84.36 | 4.46 | 101.41 | 5.09 |
| | 5.41 | 0.00 | 9.93 | 0.00 | 20.4 | 0.00 |
| | 8.68 | 0.38 | 40.66 | 1.54 | 46.43 | 1.25 |
| | 9.39 | 0.32 | 70.55 | 2.09 | 151.37 | 7.40 |
| | 0.18 | 0.06 | 1.18 | 0.22 | 0.78 | 0.34 |
| | 0.47 | 0.02 | 4.53 | 0.10 | 10.41 | 0.16 |
| Average | 10.61 | 0.35 | 31.74 | 1.28 | 53.69 | 2.71 |
| TAU_ITR165 | 4.74 | 0.13 | 25.21 | 0.69 | 47.53 | 0.90 |
| | 0.46 | 0.05 | — | 0.00 | 0.75 | 0.03 |
| | 0.31 | 0.01 | — | 0.00 | 1.16 | 0.05 |
| | 2.02 | 0.03 | 53.61 | 0.67 | 41.26 | 0.49 |
| | 7.38 | 0.15 | 30.78 | 0.53 | 42.07 | 0.58 |
| | 1.66 | 0.02 | 17.33 | 0.24 | 12.04 | 0.10 |
| Average | 2.76 | 0.06 | 31.73 | 0.35 | 24.14 | 0.36 |
| TAU_ITR162 | 8.5 | 0.75 | 29.97 | 1.37 | 63.13 | 3.01 |
| | 9.7 | 0.45 | 108.83 | 2.43 | 60 | 1.05 |
| | 52.26 | 2.43 | 119.14 | 6.08 | 91.33 | 6.73 |
| | 34.43 | 0.56 | 86.61 | 1.73 | 102.92 | 0.72 |
| | 11.48 | 0.88 | 25.31 | 1.58 | 58.59 | 4.16 |
| | 26.63 | 1.51 | 108.26 | 2.82 | 61.24 | 2.76 |
| | — | — | 29.97 | — | — | — |
| Average | 23.83 | 1.10 | 79.69 | 2.67 | 72.87 | 3.07 |
| Vehicle (PBS) | 0 | 0 | 0 | 0 | 0 | 0 |

PT3 scFv was observed in the hippocampus, cortex, olfactory bulb, thalamus, brainstem and spinal cord tissue of the mice dosed with PT3 scFv vectors. Higher PT3 scFv levels were observed with TAU_ITR62 with the ssAAV CAG PT3 VL G4S-3 VH HA rBGpA format as compared to the other two constructs.

In the serum and CSF samples collected on the terminal day, quantification of the PT3 antibody expression demonstrated enhanced expression of PT3 when delivered with VOY101. Consistent with other tissues, higher PT3 scFv levels were observed with TAU_ITR162 with the ssAAV CAG PT3 VL G4S-3 VH HA rBGpA as compared to the other two constructs. These results are shown in Table 157.

TABLE 157
PT3 expression in CSF and Serum

| | |
|---|---|
| CSF | Serum |

| Constructs | ng/ml | nM scFv | ng/ml | nM scFv |
|---|---|---|---|---|
| TAU_ITR168 | 41.65 | 1.67 | 0 | 0.00 |
| | 60.35 | 2.41 | — | 0.00 |
| | 53.1 | 2.12 | — | 0.00 |
| | 65.8 | 0.26 | — | 0.00 |
| | 6.4 | — | — | |
| Average | 45.46 | 1.62 | 0 | 0 |
| TAU_ITR165 | 79.75 | 3.19 | 120.25 | 4.81 |
| | 19.5 | 0.78 | 89.00 | 3.56 |
| | 85.2 | 3.41 | 22.20 | 0.89 |
| | — | — | 81.75 | 3.27 |
| Average | 61.48 | 2.46 | 78.30 | 3.13 |
| TAU_ITR162 | 66.3 | 2.65 | 78.15 | 3.13 |
| | 151.8 | 6.07 | 63.80 | 2.55 |
| | 166.1 | 6.64 | 111.15 | 4.45 |
| | 97.55 | 3.90 | 202.10 | 8.08 |
| | 227.35 | 9.09 | 127.90 | 5.12 |
| | — | — | 147.50 | 5.9 |
| Average | 141.82 | 5.67 | 121.80 | 4.87 |
| Vehicle (PBS) | 0 | 0 | 0 | 0 |

Liver and spleen samples collected on the terminal day, showed low PT3 antibody expression and biodistribution of AAV genomes when delivered with VOY101. The results are shown in Table 158.

TABLE 158
Vector genome quantification and
PT3 expression in Liver & Spleen

| | Liver | | Spleen | |
|---|---|---|---|---|
| Construct | Genome/cell | scFv ng/mg | Genome/cell | scFv/mg |
| TAU_ITR168 | 1.05 | 2.95 | 1.42 | 1.55 |
| | 1.75 | 2.51 | 7.70 | 1.62 |
| | 0.09 | 1.90 | 2.35 | 1.6 |
| | 0.77 | 1.90 | 4.07 | 2.11 |
| | 1.06 | 1.64 | 22.68 | 1.73 |
| | 0.02 | 2.37 | 0.52 | 2.65 |
| | 0.05 | 2.24 | 18.72 | — |
| Average | 0.68 | 2.22 | 8.21 | 1.88 |
| TAU_ITR165 | 5.31 | 16.11 | 59.87 | 1.56 |
| | 0.07 | 2.66 | 8.09 | 2.01 |
| | 0.57 | 4.25 | 4.40 | 1.85 |
| | 1.94 | 4.28 | 34.42 | 2.86 |
| | 1.50 | 5.54 | 76.93 | 1.41 |
| | 1.34 | 4.85 | 14.45 | 1.79 |
| Average | 1.79 | 6.28 | 33.03 | 1.91 |
| TAU_ITR162 | 1.50 | 5.87 | 16.52 | 2.37 |
| | 0.76 | 6.64 | 2.43 | 1.73 |
| | 2.44 | 12.15 | 2.69 | 2.12 |
| | 3.54 | 24.34 | 4.68 | 1.96 |
| | 2.85 | 19.20 | 2.60 | 2.12 |
| | 2.68 | 13.63 | 8.39 | 1.87 |
| | — | — | — | 0.97 |
| Average | 2.29 | 13.64 | 6.22 | 1.88 |
| Vehicle | 0 | 2.29 | 0 | 1.48 |
| | — | 1.78 | — | 1.6 |
| | — | 2.22 | — | 1.53 |
| | — | 1.77 | — | 1.19 |
| Average | 0 | 2.02 | 0 | 1.45 |

In comparison to CNS tissues, significantly lower AAV biodistribution was seen in peripheral tissues such as liver and spleen.

Brain tissue samples were collected on the terminal day and fixed with 4% PFA for immunohistochemistry. Anti-IgG immunohistochemistry of brain tissue collected from mice injected with VOY101.PT3 scFv AAV particles driven by CAG, GFAP or Syn promoters showed broad antibody expression across mouse CNS, but stronger and broader staining pattern was observed in the tissue of mice treated with VOY101.PT3 AAV particles driven by CAG promoter. Vehicle-treated control animals exhibited low background levels of staining. Expression of PT3 was also observed in the spinal cord.

Taken together, this study shows that PT3 is expressed in CNS tissues of all the experimental groups, with highest expression driven by the CAG promoter and cell-specific expression when driven by GFAP or synapsin promoters.

The PT3 antibody expression levels in the brain (hippocampus and cortex) and the CSF obtained with an scFv (light chain-linker-heavy chain) were compared to the full length PT3 antibody (light chain-linker-heavy chain). The PT3 levels in the cortex of mice dosed with full length antibody was similar to mice dosed with PT3 scFv. In the CSF, PT3 levels were higher in mice dosed with the full-length antibody as compared to the scFv. These observations were consistent across the different promoters tested i.e. CAG, SYN, and GFAP.

The PT3 antibody expression levels in the brain (hippocampus and cortex) and the CSF obtained with an scFv (light chain-linker-heavy chain) were compared to the full length PT3 antibody (heavy chain-linker-light chain). The PT3 expression levels in the brain and CSF of mice dosed with full length antibody was higher than the levels in mice dosed with PT3 scFv. In the CSF, PT3 levels were higher in mice dosed with the full-length antibody compared to the scFv. These observations were consistent across the different promoters tested i.e. CAG, SYN, and GFAP.

### Example 20: VOY101.PT3 Promoter and Viral Genome Configuration Studies in Rodents

### Ubiquitous Promoters and T2A or F2 Linkers for PT3 Expression

To determine the effects of promoters and viral genome configurations on brain distribution, cellular tropism and expression levels of anti-tau antibody PT3 after intravenous delivery, single-stranded viral genomes described in Table 159 were packaged into VOY101 capsid. AAV particles were formulated in 0.001% F-68 and PBS and provided intravenously at a dose of 1.4×10¹³ vg/kg into 2 month-old female C57Bl/6 mice. Twenty-eight days after administration mice were sacrificed, perfused intracardially and CSF, serum, and/or tissue samples (hippocampus, cortex, thalamus, brain stem, spinal cord, olfactory bulb, liver, spleen, skeletal muscle) were collected for analysis. Study design is shown in Table 159 where N=6 for each AAV particle tested.

TABLE 159
PT3 IV study design

| | | SEQ | | | | | End |
|---|---|---|---|---|---|---|---|
| | TAU_ITR | ID | | Dose | Dose | | of |
| AAV Particle | ID | NO: | μL | (vg/kg) | (vg) | Route | study |
| Voy101.CAG.PT3.H.F.T2A.L | TAU_ITR112 | 2162 | 100 | 1.4 × 10¹³ | 2.8 × 10¹¹ | IV | Day 28 |
| Voy101.CAG.PT3.LH.T2 | TAU_ITR116 | 2166 | 100 | 1.4 × 10¹³ | 2.8 × 10¹¹ | IV | Day 28 |
| Voy101.CBA.PT3.H.F.T2A.L | TAU_ITR111 | 2161 | 100 | 1.4 × 10¹³ | 2.8 × 10¹¹ | IV | Day 28 |
| Voy101.CBA.PT3.LH.T2A | TAU_ITR115 | 2165 | 100 | 1.4 × 10¹³ | 2.8 × 10¹¹ | IV | Day 28 |
| Voy101.CAG.PT3.HL.F2 | TAU_ITR104 | 2154 | 100 | 1.4 × 10¹³ | 2.8 × 10¹¹ | IV | Day 28 |
| Voy101.CAG.PT3.LH.F2 | TAU_ITR108 | 2158 | 100 | 1.4 × 10¹³ | 2.8 × 10¹¹ | IV | Day 28 |
| Voy101.CBA.PT3.HL.F2A | TAU_ITR103 | 2153 | 100 | 1.4 × 10¹³ | 2.8 × 10¹¹ | IV | Day 28 |
| Voy101.CBA.PT3.LH.F2A | TAU_ITR107 | 2157 | 100 | 1.4 × 10¹³ | 2.8 × 10¹¹ | IV | Day 28 |

Vector genome was quantified for a subset of tissues, such as for brain, CSF, and/or peripheral tissue (e.g. liver and spleen) by droplet digital PCR and normalized per diploid cell (VG/DC) using the endogenous mouse transferrin receptor gene (TFRC). Antibody levels in the hippocampus, cortex, olfactory bulb, brainstem, spinal cord, liver, serum, and CSF were quantified using ePHF ELISA with an anti-IgG as the detection antibody, as described above. The VG/DC values for the olfactory bulb (OB), cortex (CTX), hippocampus (HC), thalamus (THL), brainstem (BS), spinal cord (SC), spleen (SPL), liver (LIV) and muscle (MUS) are shown in Table 160. Average vector genome biodistribution quantification (VG/DC) in brain and spinal cord are shown in Table 161 and the peripheral tissues are shown in Table 162.

TABLE 160
Vector genome quantification of VOY101.PT3 constructs (AAV genomes/cell)

| | TAU_ITR112 | TAU_ITR116 | TAU_ITR111 | TAU_ITR115 |
|---|---|---|---|---|
| Tissue | (CAG.HFT2AL) | (CAG.LHT2A) | (CBA.HFT2AL) | (CBA.LHT2A) |
| OB | 11.03 | 25.28 | 11.41 | 15.42 |
| | 6.39 | 11.83 | 27.20 | 11.68 |
| | 11.33 | 20.58 | 36.29 | 18.50 |
| | 17.81 | 14.39 | 14.72 | 20.07 |
| | 8.61 | 17.70 | 46.05 | 14.78 |
| | 5.09 | 27.09 | 12.60 | — |
| | 11.69 | — | 26.53 | — |
| | 11.47 | — | 24.81 | — |
| | 13.20 | — | — | — |
| CTX | 17.56 | 29.89 | 12.05 | 22.36 |
| | 14.16 | 13.84 | 39.46 | 14.14 |
| | 21.45 | 18.43 | 35.00 | 25.34 |
| | 19.58 | 18.94 | 37.14 | 26.28 |
| | 11.55 | 18.12 | 42.07 | 21.75 |
| | 8.47 | 13.02 | 31.06 | — |
| | 14.59 | — | 2.6.68 | — |
| | 12.84 | — | 28.73 | — |
| | 14.91 | — | — | — |
| HC | 13.70 | 19.93 | 7.14 | 15.12 |
| | 27.76 | 17.47 | 20.19 | 25.90 |
| | 10.69 | 19.13 | 19.35 | 25.69 |
| | 20.17 | 11.60 | 18.99 | 23.79 |
| | 10.38 | 14.22 | 21.24 | 15.54 |
| | 14.94 | 17.37 | 16.59 | — |
| | 10.32 | — | 18.77 | — |
| | 13.46 | — | 20.62 | — |
| | 8.39 | — | — | — |
| THL | 25.10 | 38.02 | 14.29 | 43.81 |
| | 23.11 | 37.28 | 50.31 | 38.69 |
| | 15.01 | 57.03 | 42.69 | 62.34 |
| | 26.77 | 43.54 | 52.98 | 64.33 |
| | 40.46 | 30.53 | 42.49 | 29.84 |
| | 19.23 | 24.86 | 20.92 | — |
| | 19.53 | — | 22.68 | — |
| | 26.68 | — | 26.50 | — |
| | 22.20 | — | — | — |
| BS | 24.15 | 68.89 | 19.56 | 60.15 |
| | 26.46 | 45.99 | 60.70 | 34.52 |
| | 74.69 | 47.46 | 64.97 | 87.17 |
| | 26.54 | 43.39 | 66.11 | 43.45 |
| | 9.53 | 50.89 | 45.11 | 44.2.1 |
| | 17.27 | 46.93 | 33.47 | — |
| | 42.60 | — | 39.50 | — |
| | 22.49 | — | 41.19 | — |
| | 22.67 | — | — | — |
| SC | 16.17 | 36.43 | 8.40 | 33.2.1 |
| | 15.00 | 23.51 | 33.91 | 25.42 |
| | 41.81 | 46.00 | 30.21 | 85.83 |
| | 23.25 | 28.88 | 33.69 | 59.40 |
| | 11.11 | 25.02 | 30.41 | 21.07 |
| | 11.43 | 32.08 | 28.01 | — |
| | 15.34 | — | 16.43 | — |
| | 21.09 | — | 35.39 | — |
| | 12.89 | — | — | — |
| Spl | 0.81 | 0.33 | 0.35 | 0.33 |
| | 0.85 | 0.21 | 0.78 | 0.40 |
| | 0.30 | 0.21 | 0.62 | 0.78 |
| | 1.88 | 0.32 | 1.38 | 1.18 |
| | 2.03 | 0.40 | 4.05 | 0.35 |
| | 0.16 | 0.40 | 0.44 | — |
| | 0.00 | — | 0.37 | — |
| | 1.72 | — | 1.20 | — |
| | 0.35 | — | — | — |
| Liv | 2.10 | 4.33 | 10.04 | 5.96 |
| | 3.78 | 1.76 | 7.99 | 1.92 |
| | 3.12 | 4.54 | 6.03 | 2.20 |
| | 3.23 | 2.28 | 42.83 | 4.63 |
| | 2.57 | 3.17 | 27.63 | 2.57 |
| | 0.72 | 3.19 | 8.98 | — |
| | 2.89 | — | 14.96 | — |
| | 3.26 | — | 19.22 | — |
| | 3.00 | — | — | — |
| Mus | 1.39 | 0.45 | 1.09 | 0.58 |
| | 0.84 | 0.44 | 2.80 | 0.58 |
| | 0.72 | 0.46 | 0.74 | 0.54 |
| | 0.62 | 0.64 | 1.98 | 0.59 |
| | 0.23 | 0.43 | 3.56 | 0.35 |
| | 0.30 | 0.34 | 1.94 | — |
| | 0.37 | — | 0.56 | — |
| | 0.85 | — | 0.96 | — |
| | 0.49 | — | — | — |

| | TAU_ITR104 | TAU_ITR108 | TAU_ITR103 | TAU_ITR107 | |
|---|---|---|---|---|---|
| Tissue | (CAG.HLF2A) | (CAG.LHF2A) | (CBA.HLF2A) | (CBA.LHF2A) | PBS |
| OB | 18.95 | 10.14 | 13.81 | 14.12 | 0.00 |
| | 8.90 | 10.56 | 12.24 | 17.31 | 0.00 |
| | 9.60 | 23.73 | 15.78 | 13.45 | 0.00 |
| | 7.80 | 17.21 | 8.70 | 8.02 | 0.02 |
| | 25.44 | 7.90 | 11.72 | 17.90 | 0.00 |
| | 10.44 | 6.16 | 7.72 | 12.12 | 0.00 |
| | — | — | — | — | 0.00 |
| | — | — | — | — | 0.00 |
| | — | — | — | — | 0.00 |
| CTX | 18.27 | 16.27 | 22.98 | 25.81 | 0.00 |
| | 7.86 | 14.10 | 20.93 | 27.91 | 0.00 |
| | 13.77 | 19.27 | 28.47 | 22.83 | 0.00 |
| | 8.64 | 12.47 | 15.52 | 16.17 | 0.00 |
| | 22.10 | 10.96 | 11.53 | 22.20 | 0.00 |
| | 10.2.2 | 16.54 | 14.09 | 13.69 | 0.00 |
| | — | — | — | — | 0.01 |
| | — | — | — | — | 0.01 |
| | — | — | — | — | 0.00 |
| HC | 9.01 | 11.68 | 34.88 | 20.23 | 0.00 |
| | 6.74 | 11.11 | 2.3.17 | 13.35 | 0.00 |
| | 8.95 | 15.29 | 18.13 | 17.42 | 0.00 |
| | 11.38 | 16.42 | 10.82 | 17.67 | 0.00 |
| | 18.88 | 11.06 | 9.66 | 16.64 | 0.00 |
| | 5.98 | 13.97 | 10.95 | 18.70 | 0.09 |
| | — | — | — | — | 0.02 |
| | — | — | — | — | 0.00 |
| | — | — | — | — | 0.00 |
| THL | 43.59 | 39.30 | 43.77 | 44.78 | 0.00 |
| | 18.75 | 25.89 | 52.01 | 36.63 | 0.01 |
| | 22.00 | 41.03 | 60.42 | 49.85 | 0.00 |
| | 15.47 | 32.80 | 25.53 | 29.12 | 0.00 |
| | 37.53 | 22.05 | 25.34 | 62.93 | 0.01 |
| | 16.87 | 24.58 | 28.79 | 32.24 | 0.00 |
| | — | — | — | — | 0.02 |
| | — | — | — | — | 0.00 |
| | — | — | — | — | 0.01 |
| BS | 43.71 | 28.49 | 73.24 | 45.45 | 0.00 |
| | 15.25 | 34.29 | 68.12 | 47.87 | 0.00 |
| | 17.28 | 48.69 | 68.56 | 39.22 | 0.00 |
| | 21.43 | 40.09 | 29.48 | 36.99 | 0.00 |
| | 32.35 | 30.47 | 31.52 | 74.04 | 0.03 |
| | 10.73 | 52.17 | 26.65 | 38.15 | 0.00 |
| | — | — | — | — | 0.00 |
| | — | — | — | — | 0.00 |
| | — | — | — | — | 0.00 |
| SC | 23.05 | 37.25 | 45.50 | 38.44 | 0.00 |
| | 9.35 | 19.61 | 43.86 | 26.11 | 0.00 |
| | 11.31 | 37.19 | 41.01 | 39.45 | 0.01 |
| | 12.65 | 16.30 | 15.14 | 42.35 | 0.01 |
| | 16.74 | 22.23 | 13.53 | 34.78 | 0.00 |
| | 8.51 | 16.52 | 32.74 | 19.93 | 0.00 |
| | — | — | — | — | 0.03 |
| | — | — | — | — | 0.00 |
| | — | — | — | — | 0.00 |
| Spl | 0.43 | 0.32 | 0.46 | 0.44 | 0.00 |
| | 0.14 | 0.34 | 0.38 | 0.28 | 0.00 |
| | 0.28 | 0.63 | 0.79 | 0.43 | 0.00 |
| | 0.82 | 0.16 | 0.29 | 0.26 | 0.00 |
| | 0.15 | 0.22 | 0.29 | 0.79 | 0.00 |
| | 0.23 | 0.16 | 0.25 | 0.35 | 0.00 |
| | — | — | — | — | 0.00 |
| | — | — | — | — | 0.00 |
| | — | — | — | — | 0.00 |
| Liv | 4.29 | 2.17 | 3.64 | 5.39 | 0.00 |
| | 0.64 | 2.34 | 2.75 | 3.29 | 0.00 |
| | 5.36 | 5.37 | 4.58 | 3.00 | 0.00 |
| | 11.75 | 2.55 | 1.30 | 2.62 | 0.00 |
| | 1.81 | 1.11 | 0.95 | 3.86 | 0.00 |
| | 0.63 | 1.38 | 2.39 | 2.89 | 0.00 |
| | — | — | — | — | 0.00 |
| | — | — | — | — | 0.00 |
| | — | — | — | — | 0.00 |
| Mus | 0.62 | 0.25 | 0.66 | 1.54 | 0.00 |
| | 0.25 | 0.19 | 0.45 | 0.70 | 0.00 |
| | 0.22 | 0.53 | 0.67 | 0.58 | 0.00 |
| | 0.14 | 0.48 | 0.48 | 0.23 | 0.00 |
| | 0.50 | 0.18 | 0.34 | 0.47 | 0.00 |
| | 0.19 | 0.17 | 0.28 | 0.33 | 0.00 |
| | — | — | — | — | 0.00 |
| | — | — | — | — | 0.01 |
| | — | — | — | — | 0.00 |

TABLE 161
Average vector genome quantification of VOY101.PT3 constructs (AAV genomes/cell)

| | | | Olfactory | | | Spinal |
|---|---|---|---|---|---|---|
| Construct | Hippocampus | Cortex | bulb | Thalamus | Brainstem | Cord |
| TAU_ITR112 | 14.42 | 15.01 | 10.73 | 24.23 | 29.60 | 18.68 |
| (Voy101.CAG.PT3.H.F.T2A.L) | | | | | | |
| TAU_ITR116 | 16.62 | 18.71 | 19.48 | 38.54 | 50.59 | 31.99 |
| (Voy101.CAG.PT3.LH.T2) | | | | | | |
| TAU_ITR111 | 17.86 | 31.52 | 24.95 | 34.11 | 46.33 | 27.06 |
| (Voy101.CBA.PT3.H.F.T2A.L) | | | | | | |
| TAU_ITR115 | 21.21 | 21.97 | 16.09 | 47.80 | 53.90 | 44.98 |
| (Voy101.CBA.PT3.LH.T2A) | | | | | | |
| TAU_ITR104 | 10.16 | 13.48 | 13.52 | 25.70 | 23.46 | 13.60 |
| (Voy101.CAG.PT3.HL.F2) | | | | | | |
| TAU_ITR108 | 13.26 | 14.93 | 12.62 | 30.94 | 39.03 | 24.85 |
| (Voy101.CAG.PT3.LH.F2) | | | | | | |
| TAU_ITR103 | 17.94 | 18.92 | 11.66 | 39.31 | 49.60 | 31.96 |
| (Voy101.CBA.PT3.HL.F2A) | | | | | | |
| TAU_ITR107 | 17.34 | 21.44 | 13.82 | 42.59 | 46.95 | 33.51 |
| (Voy101.CBA.PT3.LH.F2A) | | | | | | |
| Vehicle (PBS) | 0.009 | 0.002 | 0.002 | 0.005 | 0.003 | 0.009 |

TABLE 162
Average vector genome quantification of VOY101.PT3
constructs (AAV genomes/cell) in peripheral tissues

| | | | Skeletal |
|---|---|---|---|
| Construct | Spleen | Liver | Muscle |
| TAU_ITR112 | 0.90 | 2.74 | 0.65 |
| (Voy101.CAG.PT3.H.F.T2A.L) | | | |
| TAU_ITR116 | 0.31 | 3.21 | 0.46 |
| (Voy101.CAG.PT3.LH.T2A) | | | |
| TAU_ITR111 | 1.15 | 17.21 | 1.71 |
| (Voy101.CBA.PT3.H.F.T2A.L) | | | |
| TAU_ITR115 | 0.61 | 3.46 | 0.53 |
| (Voy101.CBA.PT3.LH.T2A) | | | |
| TAU_ITR104 | 0.34 | 4.08 | 0.32 |
| (Voy101.CAG.PT3.HL.F2A) | | | |
| TAU_ITR108 | 0.31 | 2.49 | 0.30 |
| (Voy101.CAG.PT3.LH.F2A) | | | |
| TAU_ITR103 | 0.41 | 2.60 | 0.48 |
| (Voy101.CBA.PT3.HL.F2A) | | | |
| TAU_ITR107 | 0.42 | 3.51 | 0.64 |
| (Voy101.CBA.PT3.LH.F2A) | | | |
| Vehicle (PBS) | 0.00 | 0.00 | 0.00 |

As shown in Table 160 Table 161, and Table 162, Voy101 delivery of 8 different formats of PT3 antibody encoding viral genomes comprising CAG or CBA promoters, and T2A or F2A cleavage sites showed biodistribution in all of the 6 tested CNS tissues. Biodistribution (e.g., vector genome levels) in the olfactory bulb, cortex, hippocampus, thalamus, brainstem, and spinal cord was similar for viral genomes comprising a T2A or F2A linker region. Biodistribution of viral genomes comprising a CBA promoter (e.g., CBA.HL and CBA.LH) trended towards slightly higher AAV genomes per cell within tested CNS tissues as compared to other viral genomes comprising CAG promoters. Biodistribution to peripheral tissues (liver, spleen, skeletal muscle) was low in comparison to that of CNS tissues, with the exception of TAU_ITR111 (Voy101.CBA.PT3.H.F.T2A.L).

Antibody levels in the hippocampus, cortex, olfactory bulb, brainstem, spinal cord, liver, serum, and CSF were quantified using ePHF ELISA, as described above. Samples of hippocampus (HC), cortex (MT), olfactory bulb (OB), thalamus (TH), brain stem (BS) and spinal cord (SC) were analyzed both as ng/mg of protein and as normalized scFv/vg. The results are shown in Table 163 and 164, respectively.

TABLE 163
PT3 expression in the CNS (ng PT3/mg protein)

| | | TAU_ITR112 | TAU_ITR116 | TAU_ITR111 | TAU_ITR115 |
|---|---|---|---|---|---|
| | PBS | (CAG.HFT2AL) | (CAG.LHT2A) | (CBA.HFT2AL) | (CBA.LHT2A) |
| HC | 0 | 44.29 | 23.58 | 121.11 | 8.26 |
| | 0 | 64.60 | 37.20 | 4.91 | 7.98 |
| | 0 | 67.03 | 20.33 | 70.54 | 13.73 |
| | 0 | 41.33 | 16.14 | 25.11 | 24.88 |
| | 0 | 30.86 | 17.78 | 56.28 | 9.11 |
| | 0 | 30.13 | 12.12 | 101.49 | |
| | 0 | 22.61 | 18.39 | 31.12 | — |
| | 0 | 38.08 | — | 35.02 | — |
| | 0 | 40.65 | — | — | — |
| Avg. | 0 | 42.18 | 20.79 | 55.70 | 12.79 |
| CTX | 0 | 162.39 | 36.52 | 36.86 | 20.30 |
| | 0 | 136.13 | 73.90 | 192.78 | 12.00 |
| | 0 | 136.59 | 23.30 | 151.37 | 25.51 |
| | 0 | 94.26 | 44.09 | 140.71 | 30.80 |
| | 0 | 86.71 | 52.79 | 171.14 | 9.03 |
| | 0 | 25.35 | 23.43 | 209.27 | — |
| | 0 | 74.49 | 23.40 | 136.05 | — |
| | 0 | 118.77 | — | 167.23 | — |
| | 0 | 43.03 | — | — | — |
| Avg. | 0 | 97.52 | 39.63 | 150.68 | 19.53 |
| OB | 0 | 105.24 | 38.26 | 76.91 | 28.74 |
| | 0 | 96.03 | 60.60 | 155.09 | 24.62 |
| | 0 | 99.53 | 29.97 | 155.12 | 43.93 |
| | 0 | 103.88 | 51.72 | 149.64 | 39.86 |
| | 0 | 82.33 | 49.53 | 185.73 | 27.87 |
| | 0 | 51.29 | 38.77 | 176.50 | — |
| | 0 | 41.51 | 53.10 | 188.16 | — |
| | 0 | 136.42 | — | 185.63 | — |
| Avg. | 0 | 89.53 | 45.99 | 159.10 | 33.00 |
| TH | 0 | 54.31 | 28.08 | 166.86 | 24.87 |
| | 0 | 114.47 | 21.85 | 58.25 | 6.19 |
| | 0 | 93.00 | 43.95 | 91.59 | 32.54 |
| | 0 | 110.48 | 32.15 | 0.00 | 38.86 |
| | 0 | 80.56 | 29.50 | 191.55 | 31.67 |
| | 0 | 47.57 | 30.58 | 164.90 | — |
| | 0 | 47.56 | 0.00 | 60.75 | — |
| | 0 | 75.94 | — | 67.97 | — |
| | 0 | 19.08 | — | | — |
| Avg. | 0 | 71.44 | 26.59 | 100.23 | 26.83 |
| BS | 0 | 96.35 | 113.27 | 154.88 | 26.64 |
| | 0 | 178.75 | 144.25 | 129.38 | 29.38 |
| | 0 | 150.65 | 71.36 | 175.86 | 70.11 |
| | 0 | 154.01 | 99.21 | 91.46 | 55.75 |
| | 0 | 51.52 | 88.46 | 64.75 | 62.32 |
| | 0 | 81.80 | 60.27 | | — |
| | 0 | 130.97 | 71.52 | 85.86 | — |
| | 0 | 194.08 | — | 141.13 | — |
| | 0 | 121.91 | — | — | — |
| Avg. | 0 | 128.89 | 92.62 | 117.63 | 48.84 |
| SC | 0 | 36.66 | 21.72 | 21.54 | 17.70 |
| | 0 | 60.48 | 42.48 | 58.99 | 6.43 |
| | 0 | 47.93 | 21.91 | 79.29 | 35.65 |
| | 0 | 53.22 | 24.96 | 66.36 | 33.10 |
| | 0 | 27.91 | 18.86 | 79.45 | 13.42 |
| | 0 | 34.85 | 14.87 | 70.11 | — |
| | 0 | 47.83 | 5.48 | 84.88 | — |
| | 0 | 34.29 | 34.29 | 18.70 | — |
| | 0 | 40.89 | 40.89 | — | — |
| Avg. | 0 | 42.67 | 25.05 | 59.91 | 21.26 |
| CSF | 0 | 349.85 | 238.12 | 267.36 | 257.72 |
| | 0 | 603.34 | 329.97 | 787.22 | 340.07 |
| | 0 | 192.86 | 322.85 | 511.96 | 143.44 |
| | 0 | 381.06 | 202.54 | 662.55 | — |
| | 0 | 291.25 | 383.93 | 930.65 | — |
| | 0 | 348.83 | 270.43 | 381.43 | — |
| | 0 | 265.81 | 216.28 | 636.45 | — |
| | 0 | 287.05 | — | — | — |
| | | 325.31 | — | — | — |
| Avg. | 0 | 338.37 | 280.59 | 596.80 | 247.08 |

| | TAU_ITR104 | TAU_ITR108 | TAU_ITR103 | TAU_ITR107 |
|---|---|---|---|---|
| | (CAG.HLF2A) | (CAG.LHF2A) | (CBA.HLF2A) | (CBA.LHF2A) |
| HC | 1.26 | 13.99 | 16.56 | 11.46 |
| | 7.30 | 3.27 | 7.16 | 6.42 |
| | 0.15 | 13.37 | 4.88 | 18.50 |
| | 2.98 | 5.75 | 1.87 | 6.23 |
| | 3.28 | 39.23 | 1.11 | 8.87 |
| | 1.25 | 5.77 | 1.35 | 6.61 |
| | — | — | — | — |
| | — | — | — | — |
| | — | — | — | — |
| Avg. | 2.70 | 13.56 | 5.49 | 9.68 |
| CTX | 3.00 | 20.35 | 17.95 | 18.61 |
| | 17.39 | 13.22 | 6.78 | 18.41 |
| | 3.19 | 21.23 | 7.01 | 15.67 |
| | 7.64 | 14.35 | 4.61 | 12.72 |
| | 5.70 | 4.81 | 2.68 | 12.49 |
| | 11.84 | 10.05 | 3.15 | 7.05 |
| | 4.24 | | | |
| | — | — | — | — |
| | — | — | — | — |
| Avg. | 7.57 | 14.00 | 7.03 | 14.16 |
| OB | 14.85 | 32.68 | 22.55 | 33.63 |
| | 22.39 | 29.97 | 15.84 | 41.39 |
| | 11.39 | 45.36 | 15.39 | 39.07 |
| | 21.82 | 26.57 | 11.62 | 30.94 |
| | 24.33 | 19.15 | 7.12 | 37.23 |
| | 15.41 | 27.89 | 10.54 | 19.54 |
| | — | — | — | — |
| | — | — | — | — |
| Avg. | 18.36 | 30.27 | 13.85 | 33.63 |
| TH | 10.10 | 26.36 | 9.17 | 13.04 |
| | 21.49 | 26.55 | 15.69 | 28.23 |
| | 5.56 | 0.00 | 14.88 | 26.96 |
| | 20.06 | 22.25 | 9.35 | 15.55 |
| | 14.70 | 11.67 | 4.39 | 26.03 |
| | 15.78 | 29.91 | 8.34 | 9.72 |
| | — | — | — | — |
| | — | — | — | — |
| | — | — | — | — |
| Avg. | 14.62 | 19.46 | 10.30 | 19.92 |
| BS | 31.58 | 83.64 | 33.33 | 57.98 |
| | 51.31 | 50.38 | 45.30 | 57.08 |
| | 19.21 | 84.23 | 36.80 | 51.79 |
| | 52.13 | 32.31 | 18.90 | 18.01 |
| | 28.00 | 24.11 | 15.56 | 36.49 |
| | 6.3.16 | 44.78 | 18.15 | 16.59 |
| | — | — | — | — |
| | — | — | — | — |
| | — | — | — | — |
| Avg. | 40.90 | 53.24 | 28.01 | 39.66 |
| SC | 2.67 | 14.41 | 10.24 | 20.56 |
| | 9.28 | 18.99 | 13.38 | 17.82 |
| | 5.19 | 35.21 | 10.18 | 18.76 |
| | 2.09 | 12.01 | 6.87 | 3.45 |
| | 3.94 | 7.50 | 2.60 | 19.14 |
| | 5.34 | 20.52 | 8.43 | 6.16 |
| | — | — | — | — |
| | — | — | — | — |
| | — | — | — | — |
| Avg. | 4.75 | 18.11 | 8.62 | 14.32 |
| CSF | 0.00 | 96.29 | 62.69 | 23.36 |
| | 15.41 | 26.58 | 90.95 | 76.84 |
| | 44.17 | 42.68 | 29.47 | 64.18 |
| | 93.11 | 143.84 | 20.43 | 48.76 |
| | — | 0.00 | 41.20 | 61.42 |
| | — | — | — | 20.98 |
| | — | — | — | — |
| | — | — | — | — |
| | — | — | — | — |
| Avg. | 38.17 | 61.88 | 48.95 | 49.26 |

TABLE 164
Normalized PT3 expression in the CNS (PT3/vg)

| | | | Olfactory | | Brain | Spinal |
|---|---|---|---|---|---|---|
| | Hippocampus | Cortex | Bulb | Thalamus | stem | cord |
| TAU_ITR112 | 3.23 | 9.25 | 9.54 | 2.16 | 3.99 | 2.27 |
| (CAG.HFT2AL) | 2.33 | 9.61 | 15.03 | 4.95 | 6.76 | 4.03 |
| | 6.27 | 6.37 | 8.78 | 6.20 | 2.02 | 1.15 |
| | 2.05 | 4.81 | 5.83 | 4.13 | 5.80 | 2.29 |
| | 2.97 | 7.51 | 9.56 | 1.99 | 5.41 | 2.51 |
| | 2.02 | 2.99 | 10.08 | 2.47 | 4.74 | 3.05 |
| | 2.19 | 5.11 | 3.55 | 2.44 | 3.07 | 3.12 |
| | 2.83 | 9.25 | 11.90 | 2.85 | 8.63 | 1.63 |
| | — | 2.89 | — | 0.86 | 5.38 | 3.17 |
| Avg. | 2.99 | 6.42 | 9.28 | 3.12 | 5.09 | 2.58 |
| TAU_ITR104 | 0.14 | 0.95 | 0.78 | 0.53 | 0.72 | 0.12 |
| (CAG.HLF2A) | 1.08 | 0.41 | 2.52 | 2.42 | 3.37 | 0.99 |
| | 0.02 | 0.55 | 1.19 | 0.58 | 1.11 | 0.46 |
| | 0.26 | 0.66 | 2.80 | 2.57 | 2.43 | 0.16 |
| | 0.17 | 0.54 | 0.96 | 0.58 | 0.87 | 0.24 |
| | 0.21 | 0.41 | 1.48 | 1.51 | 5.89 | 0.63 |
| Avg. | 0.31 | 0.59 | 1.62 | 1.37 | 2.40 | 0.43 |
| TAU_ITR116 | 1.87 | 2.47 | 1.51 | 1.11 | 1.64 | 0.60 |
| (CAG.LHT2A) | 1.16 | 1.68 | 5.12 | 1.85 | 3.14 | 1.81 |
| | 0.84 | 2.39 | 1.46 | 2.14 | 1.50 | 0.48 |
| | 1.53 | 2.79 | 3.59 | 2.23 | 2.29 | 0.86 |
| | 0.85 | 1.29 | 2.80 | 1.67 | 1.74 | 0.75 |
| | 1.06 | 1.80 | 1.43 | 1.13 | 1.28 | 0.46 |
| Avg. | 1.22 | 2.07 | 2.65 | 1.69 | 1.93 | 0.83 |
| TAU_ITR108 | 1.21 | 1.25 | 3.25 | 2.62 | 2.93 | 0.40 |
| (CAG.LHF2A) | 0.29 | 0.90 | 2.84 | 2.52 | 1.52 | 0.98 |
| | 0.88 | 1.10 | 1.91 | 0.00 | 1.76 | 0.93 |
| | 0.36 | 1.15 | 1.54 | 1.29 | 0.79 | 0.70 |
| | 3.25 | 0.43 | 2.44 | 1.48 | 0.80 | 0.31 |
| | 0.42 | 0.59 | 4.78 | 5.13 | 0.90 | 1.25 |
| Avg. | 1.07 | 0.90 | 2.79 | 2.17 | 1.45 | 0.76 |
| TAU_ITR111 | 16.96 | 3.06 | 6.74 | 11.68 | 7.92 | 2.56 |
| (CBA.HFT2AL) | 0.24 | 4.89 | 5.70 | 1.16 | 2.13 | 1.74 |
| | 3.65 | 4.33 | 4.27 | 2.15 | 2.71 | 2.62 |
| | 1.32 | 3.79 | 10.16 | 0.00 | 1.38 | 1.97 |
| | 2.65 | 4.07 | 4.03 | 4.51 | 1.44 | 2.61 |
| | 6.12 | 6.74 | 14.01 | 7.88 | 2.92 | 2.50 |
| | 1.66 | 5.10 | 7.09 | 2.68 | 2.17 | 5.17 |
| | — | 5.82 | 7.48 | 2.57 | 3.43 | 0.53 |
| Avg. | 4.66 | 4.72 | 7.44 | 4.08 | 3.01 | 2.46 |
| TAU_ITR103 | 0.47 | 0.78 | 1.63 | 0.21 | 0.46 | 0.23 |
| (CBA.HLF2A) | 0.31 | 0.32 | 1.29 | 0.30 | 0.67 | 0.31 |
| | 0.27 | 0.25 | 0.98 | 0.25 | 0.54 | 0.25 |
| | 0.17 | 0.30 | 1.34 | 0.37 | 0.64 | 0.45 |
| | 0.11 | 0.23 | 0.61 | 0.17 | 0.49 | 0.19 |
| | 0.12 | 0.22 | 1.37 | 0.29 | 0.68 | 0.26 |
| Avg. | 0.24 | 0.35 | 1.20 | 0.26 | 0.58 | 0.28 |
| TAU_ITR115 | 0.55 | 0.91 | 1.86 | 0.14 | 0.44 | 0.53 |
| (CBA.LHT2A) | 0.31 | 0.85 | 2.11 | 0.84 | 0.85 | 0.25 |
| | 0.53 | 1.01 | 2.37 | 0.62 | 0.80 | 0.42 |
| | 1.05 | 1.17 | 1.99 | 0.49 | 1.28 | 0.56 |
| | 0.59 | 0.42 | 1.89 | 0.00 | — | 0.64 |
| Avg. | 0.60 | 0.87 | 2.04 | 0.42 | 0.85 | 0.48 |
| TAU_ITR107 | 0.57 | 0.72 | 2.38 | 0.29 | 1.28 | 0.53 |
| (CBA.LHF2A) | 0.48 | 0.66 | 2.39 | 0.77 | 1.19 | 0.68 |
| | 1.06 | 0.69 | 2.91 | 0.54 | 1.32 | 0.48 |
| | 0.35 | 0.79 | 3.86 | 0.53 | 0.49 | 0.08 |
| | 0.53 | 0.56 | 2.08 | 0.41 | 0.49 | 0.55 |
| | 0.35 | — | 1.61 | 0.30 | — | 0.31 |
| Avg. | 0.56 | 0.68 | 2.54 | 0.48 | 0.95 | 0.44 |
| Vehicle | 0 | 0 | 0 | 0 | 0 | 0 |
| | 0 | 0 | 0 | 0 | 0 | 0 |
| | 0 | 0 | 0 | 0 | 0 | 0 |
| | 0 | 0 | 0 | 0 | 0 | 0 |
| | 0 | 0 | 0 | 0 | 0 | 0 |
| | 0 | 0 | 0 | 0 | 0 | 0 |
| | 0 | 0 | 0 | 0 | 0 | 0 |
| | 0 | 0 | 0 | 0 | 0 | 0 |
| Avg. | 0.00 | 0.00 | 0.00 | 0.00 | 0.00 | 0.00 |

Comparing the expression levels in the hippocampus to the expression in the cortex and olfactory bulb: PT3 was higher in the HL.T2A group (i.e. TAU_ITR111 (Voy101.CBA.PT3.H.F.T2A.L) and (TAU_ITR112 (Voy101.CAG.PT3.H.F.T2A.L)) than HL.F2A (TAU_ITR104 (Voy101.CAG.PT3.HL.F2) and (TAU_ITR103 (Voy101.CBA.PT3.HL.F2A)), regardless of whether a CAG or CBA promoter was used to drive expression. Based on the ePHF ELISA data for the cortex and the olfactory bulb, PT3 expression was significantly higher when driven by a CBA promoter rather than a CAG promoter regardless of the configuration of the viral genome (e.g., heavy-linker-light vs light-linker-heavy). No statistically significant differences were observed between PT3 expression driven by CBA or CAG promoter in the other tissues. In the hippocampus, thalamus and the brain stem, PT3 expression (ng/mg) was significantly higher (p value <0.005) when the rodents were transduced with TAU_ITR111 (Voy101.CBA.PT3.H.F.T2A.L) compared to rodents transduced with TAU_ITR115 (Voy101.CBA.PT3.LH.T2A). In the cortex, olfactory bulb and spinal cord, PT3 expression (ng/mg) was significantly higher when the rodents were transduced with TAU_ITR111 (Voy101.CBA.PT3.H.F.T2A.L) compared to rodents transduced with TAU_ITR115 (Voy101.CBA.PT3.LH.T2A) (p value <0.0005) and in rodents transduced with TAU_ITR112 (Voy101.CAG.PT3.H.F.T2A.L) compared to TAU_ITR116 (Voy101.CAG.PT3.LH.T2A) (p value <0.05 in the spinal cord; p value <0.005 in the cortex and olfactory bulb). In the cortex and olfactory bulb, PT3 expression (ng/mg) was significantly higher in mice treated with TAU_ITR111 (Voy101.CBA.PT3.H.F.T2A.L) compared to mice treated with TAU_ITR112 (Voy101.CAG.PT3.H.F.T2A.L) (p value <0.0005 in the olfactory bulb and p value <0.05 in the cortex).

Normalized PT3 expression was significantly increased in mice treated with TAU_ITR112 (Voy101.CAG.PT3.H.F.T2A.L) compared to mice treated with TAU_ITR116 (Voy101.CAG.PT3.LH.T2A) (p value <0.00005 in the cortex and olfactory bulb; p value <0.0005 in the brain stem and spinal cord). Similarly, normalized PT3 expression was significantly increased in mice treated with TAU_ITR111 (Voy101.CBA.PT3.H.F.T2A.L) when compared to mice treated with TAU_ITR115 (Voy101.CBA.PT3.LH.T2A) (p value <0.0005 in the cortex and spinal cord; p value <0.005 in the olfactory bulb; p value <0.05 in the thalamus). In the brain stem, normalized PT3 expression was significantly higher upon transduction of TAU_ITR112 (Voy101.CAG.PT3.H.F.T2A.L) compared to TAU_ITR111 (Voy101.CBA.PT3.H.F.T2A.L) (p value <0.05).

In CSF samples collected on the terminal day, quantification of the PT3 antibody expression demonstrated higher PT3 expression for constructs comprising T2A linkers (i.e. TAU_ITR112, TAU_ITR116, TAU_ITR111 and TAU_ITR115) than for constructs comprising F2A linkers (i.e. TAU_ITR104, TAU_ITR108, TAU_ITR103, TAU_ITR107). PT3 expression in the CSF was higher after delivery of a viral genome with CBA.HL format (i.e. TAU_ITR111) than after delivery of a viral genome with CBA.LH format (TAU_ITR115). For viral genomes with heavy-linker-light format, the CBA promoter (i.e. TAU_ITR111 and TAU_ITR103) drove greater PT3 expression than viral genomes comprising a CAG promoter (i.e. TAU_ITR112 and TAU_ITR104). No difference in expression was observed in mice transduced with VOY101 particles expressing constructs in the CAG.HL format (TAU_ITR112 and TAU_ITR104) and CAG.LH format (TAU_ITR116 and TAU_ITR108).

Immunohistochemistry was performed on brain tissue of mice treated with an IV bolus injection of ssVOY101.PT3 AAV particles comprising viral genomes encoding PT3, operably linked to CAG or CB6 promoters. PT3 (IgG) and astrocytic (S100β) markers were assessed for colocalization of PT3 with S100β positive cells. PT3 expression was detected in different brain regions of mice dosed with CAG.HL.PT3 vector, TAU_ITR112. Lower PT3 expression was observed in the brains of mice dosed with Voy101.CAG.LH.PT3 vector, TAU_ITR116. This same pattern was observed across format pairs, wherein the heavy-linker-light format yielded comparatively more staining than the light-linker-heavy format, regardless of the linker used (e.g., T2A or F2A). PT3 antibody expression also colocalized with S100β as well as cells that displayed neuronal morphology. Neuronal localization of PT3 antibody was also confirmed by immunofluorescence studies using neuronal marker NeuN which demonstrated colocalization with PT3 (IgG) in tissues obtained from mice injected with each of the constructs described in Table 159.

Comparing the PT3 expression obtained from T2A based constructs versus F2A constructs described in Table 159, showed that constructs with the HL format namely TAU_ITR112, TAU_ITR111, TAU_ITR104, and TAU_ITR103 showed higher PT3 expression compared to constructs with the LH format namely, TAU_ITR116, TAU_ITR115. TAU_ITR108 and TAU_ITR107, regardless of whether T2A or F2A was used. Generally, viral genomes comprising a T2A cleavage site, as opposed to an F2A cleavage site, showed higher levels of PT3 expression in the CNS and CSF as measured by ELISA.

Taken together, this study shows that PT3 antibody is expressed in CNS tissues of all the experimental groups, with no significant difference in PT3 levels per vector genome observed between constructs driven by the CAG promoter and the CBA promoter.

Comparing the PT3 expression levels observed in the brain (hippocampus, cortex and thalamus) obtained with PT3 constructs described in Table 159 with the IPN002 constructs described in Example 8, it was noted that in both instances, antibody expression levels were comparable and constructs with ubiquitous promoters (CAG or CBA) comprising a T2A cleavage site resulted in higher antibody (PT3 of IPN002) expression when compared to constructs with ubiquitous promoters (CAG or CBA) comprising an F2A cleavage site. IPN002 antibody expression levels were driven more robustly when a CAG promoter was used, whereas PT3 antibody expression was greater when driven by the CBA promoter. In general, IPN002 and PT3 constructs with ubiquitous promoters (CAG or CBA) that included a T2A and the antibody in the heavy chain-light chain format showed higher expression than T2A constructs with a light chain heavy format.

### Cell-Type Specific Promoters for PT3 Expression

The effect of using cell-type specific promoters GFAP or synapsin (SYN) for driving expression of PT3 from four different viral genome configurations (HF.T2AL, LT2AH, HF.F2AL or LF2AH) was assessed based on measurement of brain distribution, cellular tropism and antibody expression level. Data from this study further enabled comparison of the effects of T2A and F2A cleavage sites on PT3 antibody expression, distribution and tropism.

Viral genomes TAU_ITR113 (SEQ ID NO. 2163), TAU_ITR117 (SEQ ID NO. 2167), TAU_ITR114 (SEQ ID NO. 2164). TAU_ITR118 (SEQ ID NO. 2168), TAU_ITR105 (SEQ ID NO. 2155), TAU_ITR109 (SEQ ID NO. 2159). TAU_ITR106 (SEQ ID NO. 2156), and TAU_ITR110 (SEQ ID NO. 2160), were packaged, single stranded, into VOY101 AAV particles and formulated in PBS and 0.001% F-68. Vehicle PBS with 0.001% F-68 was used as a control. An intravenous bolus at a dose of 1.4×10¹³ vg/kg was administered via tail vein to two-month old female C57Bl/6 mice. Twenty-eight days later, mice were sacrificed by intracardiac perfusion and tissue samples collected and processed for further analysis. The right half of the brain (including olfactory bulb) and lumbar spinal cord were post-fixed with 4% PFA for immunohistochemistry. The other half of the brain (dissected into hippocampus, cortex, thalamus, brainstem) and cervical and thoracic spinal cord tissues were processed for use in ELISA assays and vector genome quantification by droplet digital PCR normalized to diploid cell number (VG/DC) using the endogenous mouse transferrin receptor gene (TFRC). Peripheral tissues from spleen, liver and skeletal muscle were also collected. Serum samples were collected at two days prior to dosing, and weekly after dosing on days 7, 14, 21 and 28 (terminal day). CSF samples were collected on the terminal day (D28). The study design is shown in Table 165 below.

TABLE 165
PT3 IV study design

| | | SEQ | | | | | | End |
|---|---|---|---|---|---|---|---|---|
| | TAU_ITR | ID | | Dose | Dose | | | of |
| Test article | ID | NO: | μl | VG/kg | Vg | route | N | study |
| GFAP.HF.T2A.L | TAU_ITR113 | 2163 | 100 | 1.40E+13 | 2.80E+11 | IV | 9 | D 28 |
| GFAP.LH.T2 | TAU_ITR117 | 2167 | 100 | 1.40E+13 | 2.80E+11 | IV | 6 | D 28 |
| SYN.HF.T2AL | TAU_ITR114 | 2164 | 100 | 1.40E+13 | 2.80E+11 | IV | 9 | D 28 |
| SYN.LH.T2 | TAU_ITR118 | 2168 | 100 | 1.40E+13 | 2.80E+11 | IV | 6 | D 28 |
| GFAP.HL.F2 | TAU_ITR105 | 2155 | 100 | 1.40E+13 | 2.80E+11 | IV | 6 | D 28 |
| GFAP.LH.F2 | TAU_ITR109 | 2159 | 100 | 1.40E+13 | 2.80E+11 | IV | 6 | D 28 |
| SYN.HL.F2 | TAU_ITR106 | 2156 | 100 | 1.40E+13 | 2.80E+11 | IV | 6 | D 28 |
| SYN.LH.F2 | TAU_ITR110 | 2160 | 100 | 1.40E+13 | 2.80E+11 | IV | 6 | D 28 |
| PBS | | — | 100 | n.a | n.a | IV | 4 | D 28 |

CNS, peripheral and serum samples were analyzed by ePHF ELISA. Brain, olfactory bulb and spinal cord tissues were assessed by immunohistochemistry (NeuN S100β, IgG), immunofluorescence and/or hematoxylin and eosin staining (H&E). Vector genome quantification using droplet digital PCR (ddPCR) was carried out for samples collected from cortex (CTX), hippocampus (HC), anterior olfactory bulb (OB), brain stem (BS), thoracic spinal cord (SC), thalamus (TH), liver, spleen, and skeletal muscle. Secondary readouts included daily cage side observations for 1 week after AAV particle administration and weekly body weight measurements. Vector genome quantifications (VG/DC) are shown in the Table 166 below.

TABLE 166
Vector genome quantification of ssVOY101.PT3 constructs

| | | TAU_ITR114 | TAU_ITR118 | TAU_ITR113 | TAU_ITR117 |
|---|---|---|---|---|---|
| | PBS | (SYN.HLT2A) | (SYN.LHT2A) | (GFAP.HLT2A) | (GFAP.LHT2A) |
| HC | 0.01 | 19.25 | 20.91 | 13.56 | 34.61 |
| | 0.01 | 51.14 | 62.89 | 10.55 | 46.43 |
| | 0.06 | 18.75 | 25.33 | 18.24 | 17.57 |
| | 0.01 | 11.39 | 63.78 | 20.10 | 26.96 |
| | 0.00 | 7.97 | 33.66 | 49.67 | 36.65 |
| | 0.00 | 14.99 | 42.15 | 14.78 | 42.71 |
| | 0.00 | 18.79 | — | 26.07 | — |
| | 0.00 | 12.87 | — | 41.19 | — |
| | — | 20.99 | — | 18.22 | — |
| Avg. | 0.01 | 19.57 | 41.45 | 23.59 | 34.16 |
| CTX | 0.00 | 14.70 | 59.18 | 24.28 | 37.16 |
| | 0.01 | 28.02 | 33.64 | 24.47 | 23.21 |
| | 0.00 | 31.61 | 25.38 | 23.90 | 24.64 |
| | 0.02 | 23.40 | 32.40 | 31.76 | 28.39 |
| | 0.00 | 7.04 | 27.33 | 36.38 | 48.54 |
| | 0.00 | 20.35 | 62.88 | 19.87 | 52.03 |
| | 0.00 | 25.98 | — | 34.53 | — |
| | 0.00 | 13.80 | — | 34.66 | — |
| | | 16.07 | — | 35.22 | — |
| Avg. | 0.00 | 20.11 | 40.13 | 29.45 | 35.66 |
| OB | 0.00 | 6.03 | 48.78 | 15.19 | 57.56 |
| | 0.00 | 20.33 | 8.38 | 3.10 | 29.66 |
| | 0.00 | 18.69 | 29.48 | 17.12 | 15.53 |
| | 0.00 | 7.16 | 29.55 | 36.82 | 28.82 |
| | 0.00 | 5.49 | 23.79 | 22.73 | 33.59 |
| | 0.00 | 14.86 | 12.97 | 15.25 | 51.84 |
| | 0.00 | 27.58 | — | 30.13 | — |
| | 0.02 | 16.35 | — | 42.10 | — |
| | | 21.47 | — | 18.78 | — |
| Avg. | 0.00 | 15.33 | 25.49 | 22.36 | 36.17 |
| TH | 0.01 | 20.40 | 229.00 | 32.09 | 41.58 |
| | 0.00 | 51.20 | 36.82 | 23.25 | 32.98 |
| | 0.06 | 40.21 | 207.63 | 30.81 | 55.41 |
| | 0.00 | 26.05 | 232.78 | 38.21 | 105.17 |
| | 0.00 | 7.82 | 184.74 | 43.81 | 41.36 |
| | 0.01 | 28.39 | 42.62 | 25.09 | 69.23 |
| | 0.00 | 27.67 | — | 42.05 | — |
| | 0.00 | 18.32 | — | 41.98 | — |
| | | 24.01 | — | 44.53 | — |
| Avg. | 0.01 | 27.12 | 155.60 | 35.76 | 57.62 |
| BS | 0.00 | 22.20 | 31.10 | 35.32 | 109.90 |
| | 0.06 | 46.25 | 102.52 | 26.03 | 38.26 |
| | 0.01 | 47.05 | 165.28 | 41.68 | 80.22 |
| | 0.02 | 26.71 | 50.57 | 53.04 | 99.61 |
| | 0.00 | 9.66 | 27.83 | 51.72 | 49.34 |
| | 0.00 | 34.54 | 50.73 | 24.94 | 132.43 |
| | 0.00 | 57.33 | — | 61.72 | — |
| | 0.00 | 34.05 | — | 51.76 | — |
| | | 52.59 | — | 53.14 | — |
| Avg. | 0.01 | 36.71 | 71.34 | 44.37 | 84.96 |
| SC | 0.02 | 12.63 | 55.71 | 20.02 | 41.26 |
| | 0.00 | 23.52 | 30.66 | 13.16 | 45.67 |
| | 0.01 | 23.18 | 234.44 | 22.87 | 66.02 |
| | 0.01 | 21.32 | 69.92 | 25.01 | 71.70 |
| | 0.00 | 4.30 | 29.48 | 93.99 | 45.59 |
| | 0.00 | 28.88 | 32.72 | 19.45 | 71.37 |
| | 0.01 | 25.01 | — | 29.22 | — |
| | 0.01 | 14.15 | — | 28.30 | — |
| | | 22.80 | — | 69.61 | — |
| Avg. | 0.01 | 19.53 | 75.49 | 35.74 | 56.93 |
| | | TAU_ITR106 | TAU_ITR110 | TAU_ITR105 | TAU_ITR109 |
| | | (SYN.HLF2A) | (SYN.LHF2A) | (GFAP.HLF2A) | (GFAP.LHF2A) |
| | HC | 23.69 | 27.95 | 27.21 | 22.27 |
| | | 27.04 | 0.26 | 25.35 | 39.38 |
| | | 35.50 | 17.85 | 25.90 | 10.75 |
| | | 128.16 | 25.89 | 28.65 | 48.17 |
| | | 28.96 | 4.91 | 29.76 | 21.96 |
| | | 29.09 | 44.57 | 18.38 | 25.36 |
| | | — | — | — | — |
| | | — | — | — | — |
| | | — | — | — | — |
| | Avg. | 45.41 | 20.24 | 25.88 | 27.98 |
| | CTX | 17.10 | 29.05 | 34.18 | 26.99 |
| | | 43.23 | 0.36 | 21.34 | 49.96 |
| | | 28.30 | 22.09 | 26.54 | 19.11 |
| | | 71.28 | 38.38 | 15.73 | 44.82 |
| | | 25.45 | 5.64 | 42.75 | 36.38 |
| | | 29.04 | 24.62 | 31.08 | 62.02 |
| | | — | — | — | — |
| | | — | — | — | — |
| | | — | — | — | — |
| | Avg. | 35.73 | 20.02 | 28.60 | 39.88 |
| | OB | 20.57 | 49.11 | 22.53 | 12.62 |
| | | 38.79 | 0.34 | 27.02 | 20.34 |
| | | 17.77 | 39.36 | 14.16 | 32.17 |
| | | 18.55 | 28.85 | 9.81 | 37.46 |
| | | 39.88 | 7.26 | 38.96 | 72.44 |
| | | 19.44 | 22.87 | 21.94 | 42.74 |
| | | — | — | — | — |
| | | — | — | — | — |
| | | — | — | — | — |
| | Avg. | 25.84 | 24.63 | 22.40 | 36.29 |
| | TH | 58.55 | 39.01 | 29.31 | 51.71 |
| | | 64.07 | 0.44 | 38.35 | 35.27 |
| | | 60.02 | 50.02 | 20.17 | 20.43 |
| | | 55.58 | 33.76 | 21.29 | 82.81 |
| | | 36.88 | 10.18 | 59.58 | 33.71 |
| | | 40.03 | 54.62 | 24.52 | 63.61 |
| | | — | — | — | — |
| | | — | — | — | — |
| | | — | — | — | — |
| | Avg. | 52.52 | 31.34 | 32.20 | 47.92 |
| | BS | 24.77 | 32.99 | 67.09 | 49.48 |
| | | 56.04 | 0.39 | 24.33 | 48.96 |
| | | 90.96 | 60.80 | 111.66 | 29.63 |
| | | 173.87 | 58.40 | 15.96 | 66.14 |
| | | 73.88 | 14.50 | 55.40 | 69.48 |
| | | 145.74 | 77.95 | 48.63 | 84.84 |
| | | — | — | — | — |
| | | — | — | — | — |
| | | — | — | — | — |
| | Avg. | 94.21 | 40.84 | 53.84 | 58.09 |
| | SC | 75.62 | 28.92 | 37.12 | 122.50 |
| | | 108.09 | 0.18 | 42.17 | 33.47 |
| | | 66.58 | 24.03 | 81.91 | 20.62 |
| | | 53.83 | 43.88 | 10.57 | 24.36 |
| | | 0.00 | 20.65 | 40.76 | 90.37 |
| | | 48.26 | 25.74 | 23.29 | 40.89 |
| | | — | — | — | — |
| | | — | — | — | — |
| | | — | — | — | — |
| | Avg. | 58.73 | 23.90 | 39.30 | 55.37 |

Across the six CNS tissues analyzed, TAU_ITR118 (SYN.LH. T2A) showed higher AAV biodistribution in hippocampus, cortex, olfactory bulb, thalamus and spinal cord. In the brain stem, TAU_ITR106 (SYN.HL. F2A) showed the highest AAV biodistribution. The remaining AAV viral genomes tested demonstrated fairly similar AAV biodistribution.

Comparing the AAV biodistribution of TAU_ITR112 (CAG.PT3.HFT2AL; SEQ ID NO: 2162) described in Table 160 with TAU_ITR113 (GFAP.PT3.HLT2A; SEQ ID NO: 2163) or TAU_ITR114 (SYN.PT3.HLT2A; SEQ ID NO: 2164), GFAP.PT3.HLT2A resulted in significantly higher expression in the cortex compared to CAG.PT3.HFT2AL (p value <0.0005, one-way ANOVA-Tukey's multiple comparison test) or SYN.PT3.HLT2A (p value <0.005). In the thalamus, AAV biodistribution in mice treated with TAU_ITR113 was significantly higher than the mice treated with TAU_ITR112 (p value <0.05). The vector genome quantification in peripheral tissues is provided in Table 167.

TABLE 167
Vector genome quantification of ssVOY101.PT3 constructs in peripheral tissues

| | | TAU_ITR114 | TAU_ITR118 | TAU_ITR113 | TAU_ITR117 |
|---|---|---|---|---|---|
| | PBS | (SYN.HLT2A) | (SYN.LHT2A) | (GFAP.HLT2A) | (GFAP.LHT2A) |
| Liver | 0.00 | 2.00 | 4.34 | 3.86 | 6.50 |
| | 0.00 | 4.03 | 3.29 | 1.73 | 6.41 |
| | 0.00 | 3.70 | 9.50 | 4.76 | 7.38 |
| | 0.00 | 5.31 | 4.78 | 2.64 | 5.81 |
| | 0.00 | 1.03 | 13.80 | 4.43 | 1.22 |
| | 0.00 | 2.06 | 3.74 | 8.77 | 8.33 |
| | 0.00 | 3.85 | — | 6.95 | — |
| | 0 00 | 2.72 | — | 6.06 | — |
| | | 3.25 | — | 1.56 | — |
| Avg. | 0.00 | 3.11 | 6.58 | 4.53 | 5.94 |
| Spleen | 0.00 | 0.92 | 0.41 | 10.97 | 7.05 |
| | 0.00 | 0.80 | 4.07 | 0.22 | 1.06 |
| | 0.00 | 15.69 | 0.59 | 14.33 | 0.81 |
| | 0.00 | 0.57 | 19.40 | 0.55 | 0.67 |
| | 0 00 | 0.19 | 5.48 | 0.59 | 0.78 |
| | 0.00 | 0.15 | 0.77 | 37.74 | 1.03 |
| | 0.00 | 5.84 | — | 8.54 | — |
| | 0.00 | 1.48 | — | 0.57 | — |
| | | 0.27 | — | 1.94 | — |
| Avg. | 0.00 | 2.88 | 5.12 | 8.38 | 1.90 |
| Skeletal | 0.00 | 0.68 | 1.78 | 1.10 | 0.95 |
| Muscle | 0.00 | 1.45 | 5.56 | 0.39 | 0.85 |
| | 0.00 | 1.07 | 0.64 | 0.93 | 0.67 |
| | 0.00 | 0.76 | 0.25 | 1.53 | 1.58 |
| | 0.00 | 0.39 | 0.29 | 2.74 | 0.81 |
| | 0.00 | 0.55 | 1.10 | 0.70 | 0.60 |
| | 0.00 | 2.21 | — | 1.05 | — |
| | 0.00 | 0.70 | — | 1.17 | — |
| | | 0.64 | — | 0.90 | — |
| Avg. | 0.00 | 0.94 | 1.60 | 1.17 | 0.91 |

| | TAU_ITR106 | TAU_ITR110 | TAU_ITR105 | TAU_ITR109 |
|---|---|---|---|---|
| | (SYN.HLF2A) | (SYN.LHF2A) | (GFAP.HLF2A) | (GFAP.LHF2A) |
| Liver | 1.91 | 3.38 | 5.32 | 1.51 |
| | 6.81 | 0.00 | 3.90 | 4.08 |
| | 6.60 | 7.60 | 8.66 | 4.04 |
| | 12.00 | 4 17 | 2.71 | 7.62 |
| | 11.65 | 1.08 | 4.69 | 8.29 |
| | 18.58 | 4.09 | 2.50 | 3.97 |
| | — | — | — | — |
| | — | — | — | — |
| | — | — | — | — |
| Avg. | 9.59 | 3.39 | 4.63 | 4.92 |
| Spleen | 1.30 | 0.43 | 32.61 | 0.56 |
| | 16.82 | 0.51 | 0.76 | 1.55 |
| | 10.11 | 4.72 | 1.21 | 0.09 |
| | 3.36 | 16.62 | 9.64 | 3.10 |
| | 5.70 | 1.20 | 0.47 | 0.82 |
| | 12.35 | 2.70 | 1.12 | 4.43 |
| | — | — | — | — |
| | — | — | — | — |
| | — | — | — | — |
| Avg. | 8.27 | 4.36 | 7.63 | 1.76 |
| Skeletal | 1.12 | 0.36 | 1.33 | 0.56 |
| Muscle | 2.03 | 0.44 | 1.24 | 0.55 |
| | 0.61 | 0.52 | 0.77 | 0.79 |
| | 0.55 | 0.47 | 0.34 | 0.46 |
| | 0.76 | 0.15 | 0.63 | 0.95 |
| | 0.43 | 0.28 | 0.39 | 1.14 |
| | — | — | — | — |
| | — | — | — | — |
| | — | — | — | — |
| Avg. | 0.92 | 0.37 | 0.78 | 0.74 |

As shown in Table 167, AAV biodistribution of the various viral genomes tested showed that the biodistribution in the peripheral tissues was much lower than the biodistribution observed in the CNS.

Samples of hippocampus, cortex, olfactory bulb, thalamus, brainstem, spinal cord, liver, spleen, skeletal muscle and serum were subjected to analysis by ePHF ELISA to determine PT3 expression from each of tbc cell-type specific PT3 viral genomes tested. Control studies were performed to confirm the standard curve and determine that the matrix effect is not observed in this ELISA assay. PT3 antibody expression was then normalized to the viral genome biodistribution (PT3/VG). Data are shown in Table 168 below as ng/mg protein (or ng/mL for serum samples) and Table 169 as PT3/vg (shortened to ab/vg).

TABLE 168
PT3 expression (ng PT3/mg protein)

| | | TAU_ITR114 | TAU_ITR118 | TAU_ITRU3 | TAU_ITR117 |
|---|---|---|---|---|---|
| | PBS | (SYN.HLT2A) | (SYN.LHT2A) | (GFAP.HLT2A) | (GFAP.LHT2A) |
| HC | 0 | 10.93 | 1.87 | 6.45 | 3.32 |
| | 0 | 27.86 | 3.05 | 14.35 | 5.18 |
| | 0 | 17.91 | 2.54 | 28.98 | 0.78 |
| | 0 | 8.17 | 4.78 | 9.06 | 1.55 |
| | 0 | −0.63 | 2.94 | 12.14 | 1.48 |
| | 0 | 17.55 | 0 | 16.30 | 3.55 |
| | 0 | 8.56 | — | 18.60 | — |
| | 0 | 19.96 | — | — | — |
| | — | 17.32 | — | — | — |
| Avg. | 0 | 14.18 | 2.53 | 15.12 | 2.64 |
| CTX | 0 | 12.15 | 6.46 | 49.00 | 10.83 |
| | 0 | 25.42 | 6.02 | 15.75 | 14.66 |
| | 0 | 23.33 | 8.47 | 41.61 | 5.17 |
| | 0 | 16.00 | 13.86 | 50.37 | 7.86 |
| | 0 | 2.24 | 8.21 | 69.70 | 12.22 |
| | 0 | 46.93 | 9.19 | 41.81 | 19.89 |
| | 0 | 56.18 | — | 68.12 | — |
| | 0 | 29.68 | — | 42.17 | — |
| Avg. | 0 | 26.49 | 8.70 | 47.32 | 11.77 |
| OB | 0 | 19.47 | 14.15 | 59.57 | 30.68 |
| | 0 | 58.45 | 19.67 | 43.29 | 53.70 |
| | 0 | 115.91 | 15.69 | 153.88 | 56.29 |
| | 0 | 79.90 | 12.41 | 151.17 | 82.15 |
| | 0 | 4.62 | 19.47 | 110.06 | 97.04 |
| | 0 | 67.05 | 24.42 | 209.14 | 133.00 |
| | 0 | 72.37 | — | 187.76 | — |
| | 0 | 54.52 | — | 233.63 | — |
| | — | 90.94 | — | 135.86 | — |
| Avg. | 0 | 62.58 | 17.64 | 142.71 | 75.48 |
| TH | 0 | 28.37 | 25.95 | 28.43 | 4.86 |
| | 0 | 89.14 | 20.82 | 6.14 | 6.34 |
| | 0 | 74.84 | 32.33 | 29.82 | 4.03 |
| | 0 | 84.59 | 25.53 | 30.61 | 2.13 |
| | 0 | 9.01 | 23.38 | 11.69 | 9.79 |
| | 0 | 39 | 4.75 | 16.15 | 14.6 |
| | 0 | 101.52 | — | 24.96 | — |
| | 0 | 21.89 | — | 16.96 | — |
| | — | 75.46 | — | — | — |
| Avg. | 0 | 58.20 | 22.13 | 20.60 | 6.96 |
| BS | 0 | 44.59 | 16.11 | 56.24 | 10.8 |
| | 0 | 79.98 | 18.31 | 33.04 | 13.05 |
| | 0 | 72.07 | 25.29 | 134.39 | 13.87 |
| | 0 | 42.39 | 17.80 | 57.02 | 9.72 |
| | 0 | 13.27 | 15.10 | 75.84 | 10.27 |
| | 0 | 58.28 | 20.09 | 37.94 | 15.51 |
| | 0 | 85.51 | — | 60.70 | — |
| | 0 | 62.06 | — | 46.41 | — |
| | — | 70.87 | — | — | — |
| Avg. | 0 | 58.78 | 18.78 | 62.70 | 12.20 |
| SC | 0 | 46.55 | 25.67 | 53.03 | 11.27 |
| | 0 | 86.71 | 44.13 | 34.72 | 15.04 |
| | 0 | 96.74 | 23.73 | 29.93 | 7.25 |
| | 0 | 73.22 | 32.70 | 45.11 | 8.27 |
| | 0 | 9.62 | 22.02 | 32.53 | 9.53 |
| | 0 | 59.44 | 13.28 | 28.94 | 16.83 |
| | 0 | 103.70 | — | 90.90 | — |
| | 0 | 79.46 | — | 44.64 | — |
| | — | 59.52 | — | 123.22 | — |
| Avg. | 0 | 68.33 | 26.92 | 53.67 | 11.37 |
| Liver | −0.09 | 0.65 | 0.34 | 8.01 | 7.51 |
| | −0.13 | 0.80 | 0.30 | 5.09 | 26.68 |
| | −0.08 | 1.00 | 0.48 | 13.78 | 42.32 |
| | −0.09 | 0.13 | 0.05 | 7.78 | 39.09 |
| | 0.00 | 1.05 | −0.27 | 11.18 | 22.21 |
| | −0.03 | 1.00 | 0.15 | 17.62 | — |
| | −0.07 | 0.89 | — | 13.36 | — |
| | −0.08 | 0.35 | — | 10.70 | — |
| Avg. | 0 | 0.73 | 0.18 | 10.94 | 27.56 |
| Spleen | 1.64 | 0.66 | 0.33 | 18.16 | 17.39 |
| | −1.28 | −0.82 | 0.14 | −4.00 | 20.10 |
| | −1.27 | 0.85 | 2.00 | 5.99 | 55.44 |
| | 0.57 | 2.62 | −0.10 | 5.21 | 36.70 |
| | −1.33 | 3.11 | 1.43 | 16.41 | 0.53 |
| | −1.52 | 0.65 | 0.19 | 6.49 | 74.95 |
| | −1.45 | 1.01 | — | 11.45 | — |
| | −2.38 | 0.75 | — | 10.07 | — |
| Avg. | −1 | 1.10 | 0.67 | 8.72 | 34.19 |
| Muscle | 0 | 0.97 | 0.74 | 1.55 | 2.46 |
| | 0 | 1.79 | 0.35 | 3.30 | 2.47 |
| | 0 | 2.58 | 0.51 | 11.39 | 2.38 |
| | 0 | 9.07 | 0.64 | 21.62 | 2.60 |
| | 0 | 1.73 | 1.88 | 30.43 | 0.62 |
| | 0 | 5.48 | 0.40 | 0.00 | 6.48 |
| | 0 | 16.19 | — | 0.00 | — |
| | 0 | 10.84 | — | 18.93 | — |
| | — | 0.72 | — | 5.03 | — |
| Avg. | 0 | 5.49 | 0.75 | 10.25 | 2.83 |

| | TAU_ITR106 | TAU_ITR110 | TAU_ITR105 | TAU_ITR109 |
|---|---|---|---|---|
| | (SYN.HLF2A) | (SYN.LHF2A) | (GFAP.HLF2A) | (GFAP.LHF2A) |
| HC | 9.11 | 1.83 | 0.54 | 0.24 |
| | 14.16 | −1.49 | 0.08 | 1.49 |
| | 5.39 | 1.57 | 0.13 | −0.59 |
| | 10.2 | 2.03 | −0.54 | 0.33 |
| | 5.8 | −0.73 | 0.16 | 0.41 |
| | 7.04 | 2.16 | −0.39 | 0.29 |
| | — | — | — | — |
| | — | — | — | — |
| | — | — | — | — |
| Avg. | 8.62 | 0.90 | 0.00 | 0.36 |
| CTX | 6.82 | 5.77 | 2.31 | 3.94 |
| | 14.79 | 0.00 | 3.14 | 7.28 |
| | 9.02 | 1.97 | 2.97 | 1.19 |
| | 9.81 | 4.11 | 0.46 | 4.05 |
| | 4.04 | 0.23 | 3.05 | 6.82 |
| | 4.60 | 5.31 | 1.24 | 5.70 |
| | — | — | — | — |
| | — | — | — | — |
| Avg. | 8.18 | 2.90 | 2.20 | 4.83 |
| OB | 19.49 | 4.15 | 23.62 | 11.92 |
| | 20.44 | 0.00 | 2.21 | 29.34 |
| | 17.12 | 6.83 | 9.99 | 14.29 |
| | 30.58 | 12.02 | 4.72 | 21.99 |
| | 12.75 | 1.29 | 14.17 | 41.27 |
| | 25.95 | 13.92 | 13.52 | 34.12 |
| | — | — | — | — |
| | — | — | — | — |
| | — | — | — | — |
| Avg. | 21.06 | 6.37 | 11.37 | 25.49 |
| TH | 10.41 | 16.13 | 3.43 | 1.19 |
| | 31.57 | 0 | 3.64 | 3.41 |
| | 29.82 | 1.05 | 1.62 | 1.3 |
| | 27.54 | 14.76 | 0 | 3.73 |
| | 15.52 | 1.69 | 0.29 | 5.25 |
| | 29.58 | 16.56 | 0.41 | 3.86 |
| | — | — | — | — |
| | — | — | — | — |
| | — | — | — | — |
| Avg. | 24.07 | 8.37 | 1.57 | 3.12 |
| BS | 24.52 | 16.05 | 0.06 | 1.43 |
| | 41.63 | 0.00 | 1.14 | 7.76 |
| | 21.17 | 8.87 | 3.40 | 3.60 |
| | 41.11 | 24.03 | 0.29 | 2.18 |
| | 32.40 | 8.36 | 4.23 | 9.39 |
| | 44.11 | 22.37 | 3.17 | 14.72 |
| | — | — | — | — |
| | — | — | — | — |
| | — | — | — | — |
| Avg. | 34.16 | 13.28 | 2.05 | 6.51 |
| SC | 33.43 | 31.66 | 10.63 | 6.52 |
| | 73.11 | 0.00 | 5.11 | 11.46 |
| | 47.52 | 14.82 | 7.47 | 3.05 |
| | 68.34 | 11.73 | 1.29 | 6.97 |
| | 52.26 | 5.95 | 5.61 | 9.52 |
| | 42.91 | 28.6 | 4.41 | 10.27 |
| | — | — | — | — |
| | — | — | — | — |
| | — | — | — | — |
| Avg. | 52.93 | 15.46 | 5.75 | 7.97 |
| Liver | 0.52 | 0.10 | 4.30 | 8.14 |
| | 0.30 | −0.04 | 3.60 | 14.99 |
| | −0.24 | −0.03 | 30.70 | 22.07 |
| | −0.03 | 0.11 | 10.27 | 22.68 |
| | 0.15 | 0.02 | 6.17 | 31.91 |
| | 0.37 | 0.13 | 0.97 | 17.71 |
| | — | — | — | — |
| | — | — | — | — |
| Avg. | 0.18 | 0.05 | 9.33 | 19.58 |
| Spleen | 0.10 | −0.34 | 4.20 | 2.24 |
| | 0.48 | −0.57 | 3.74 | 4.27 |
| | −1.23 | −0.21 | 5.56 | 29.90 |
| | 0.02 | −0.22 | 2.70 | 29.66 |
| | 0.48 | −0.72 | 3.83 | 9.22 |
| | 0.87 | −0.49 | 2.71 | 5.43 |
| | — | — | — | — |
| | — | — | — | — |
| Avg. | 0.12 | −0.42 | 3.79 | 13.45 |
| Muscle | 0.14 | 0.00 | 0.52 | 0.39 |
| | 0.40 | 0.00 | 1.0.3 | 2.9.3 |
| | 0.37 | 0.18 | 1.58 | 6.70 |
| | 0.33 | 0.21 | 0.73 | 2.41 |
| | 0.91 | 0.02 | 0.76 | 8.27 |
| | 0.46 | 0.02 | 0.99 | 18.48 |
| | — | — | — | — |
| | — | — | — | — |
| | — | — | — | — |
| Avg. | 0.44 | 0.07 | 0.93 | 6.53 |

TABLE 169
PTS expression (PT3/vg)

| | | TAU_ITR114 | TAU_ITR118 | TAU_ITR113 | TAU_ITR117 |
|---|---|---|---|---|---|
| | PBS | (SYN.HLT2A) | (SYN.LHT2A) | (GFAP.HLT2A) | (GFAP.LHT2A) |
| HC | 0 | 0.57 | 0.09 | 0.61 | 0.10 |
| | 0 | 0.54 | 0.05 | 0.79 | 0.11 |
| | 0 | 0.96 | 0.10 | 1.44 | 0.04 |
| | 0 | 0.72 | 0.07 | 0.18 | 0.06 |
| | 0 | 0.00 | 0.09 | 0.82 | 0.04 |
| | 0 | 1.17 | 0.00 | 0.63 | 0.08 |
| | 0 | 0.46 | — | 0.45 | — |
| | 0 | 1.55 | — | — | — |
| Avg. | 0 | 0.75 | 0.07 | 0.70 | 0.07 |
| CTX | 0 | 0.83 | 0.11 | 2.02 | 0.29 |
| | 0 | 0.91 | 0.18 | 0.64 | 0.63 |
| | 0 | 0.74 | 0.33 | 1.74 | 0.21 |
| | 0 | 0.68 | 0.43 | 1.59 | 0.28 |
| | 0 | 0.32 | 0.30 | 1.92 | 0.25 |
| | 0 | 2.31 | 0.15 | 2.10 | 0.38 |
| | 0 | 2.16 | — | 1.97 | — |
| | 0 | — | — | 1.22 | — |
| Avg. | 0 | 1.13 | 0.25 | 1.65 | 0.34 |
| OB | 0 | 3.23 | 0.29 | 3.92 | 0.53 |
| | 0 | 2.88 | 2.35 | 13.95 | 1.81 |
| | 0 | 6.20 | 0.53 | 8.99 | 3.62 |
| | 0 | 11.16 | 0.42 | 4.11 | 2.85 |
| | 0 | 0.84 | 0.82 | 4.84 | 2.89 |
| | 0 | 4.51 | 1.88 | 13.72 | 2.57 |
| | 0 | 2.62 | — | 6.23 | — |
| | 0 | 3.34 | — | 5.55 | — |
| | 0 | 4.24 | — | 7.23 | — |
| Avg. | 0 | 4.34 | 1.05 | 7.61 | 2.38 |
| TH | 0 | 1.39 | 0.11 | 0.89 | 0.12 |
| | 0 | 1.74 | 0.57 | 0.26 | 0.19 |
| | 0 | 1.86 | 0.16 | 0.00 | 0.07 |
| | 0 | 3.25 | 0.11 | 0.78 | 0.02 |
| | 0 | 1.15 | 0.13 | 0.70 | 0.24 |
| | 0 | 1.37 | 0.11 | 0.47 | 0.21 |
| | 0 | 3.67 | — | 0.38 | — |
| | 0 | 1.20 | — | 0.59 | — |
| | 0 | 3.14 | — | 0.38 | — |
| Avg. | 0 | 2.09 | 0.20 | 0.49 | 0.14 |
| BS | 0 | 2.01 | 0.52 | 1.59 | 0.10 |
| | 0 | 1.73 | 0.18 | 1.27 | 0.34 |
| | 0 | 1.53 | 0.15 | 3.22 | 0.17 |
| | 0 | 1.59 | 0.35 | 1.08 | 0.10 |
| | 0 | 1.37 | 0.54 | 1.47 | 0.21 |
| | 0 | 1.69 | 0.40 | 1.52 | 0.12 |
| | 0 | 1.49 | — | 0.98 | — |
| | 0 | 1.82 | — | 0.90 | — |
| | 0 | 1.35 | — | | — |
| Avg. | 0 | 1.62 | 0.36 | 1.50 | 0.17 |
| SC | 0 | 3.69 | 0.46 | 2.65 | 0.27 |
| | 0 | 3.69 | 1.44 | 2.64 | 0.33 |
| | 0 | 4.17 | 0.10 | 1.31 | 0.11 |
| | 0 | 3.43 | 0.47 | 1.80 | 0.12 |
| | 0 | 2.24 | 0.75 | 0.35 | 0.21 |
| | 0 | 2.06 | 0.41 | 1.49 | 0.24 |
| | 0 | 4.15 | — | 3.11 | — |
| | 0 | 5.62 | — | 1.58 | — |
| | 0 | 2.61 | — | 1.77 | — |
| Avg. | 0 | 3.52 | 0.60 | 1.85 | 0.21 |
| | | TAU_ITR106 | TAU_ITR110 | TAU_ITR105 | TAU_ITR109 |
| | | (SYN.HLF2A) | (SYN.LHF2A) | (GFAP.HLF2A) | (GFAP.LHF2A) |
| | HC | 0.38 | 0.07 | 0.02 | 0.01 |
| | | 0.52 | 0.00 | 0.00 | 0.04 |
| | | 0.15 | 0.09 | 0.00 | 0.00 |
| | | 0.08 | 0.08 | 0.00 | 0.01 |
| | | 0.20 | 0.00 | 0.01 | 0.02 |
| | | 0.24 | 0.05 | 0.00 | 0.01 |
| | | — | — | — | — |
| | | — | — | — | — |
| | Avg. | 0.26 | 0.05 | 0.01 | 0.01 |
| | CTX | 0.40 | 0.20 | 0.07 | 0.15 |
| | | 0.34 | 0.00 | 0.15 | 0.15 |
| | | 0.32 | 0.09 | 0.11 | 0.06 |
| | | 0.14 | 0.11 | 0.03 | 0.09 |
| | | 0.16 | 0.04 | 0.07 | 0.19 |
| | | 0.16 | 0.22 | 0.04 | 0.09 |
| | | — | — | — | — |
| | | — | — | — | — |
| | Avg. | 0.25 | 0.11 | 0.08 | 0.12 |
| | OB | 0.95 | 0.08 | 1.05 | 0.94 |
| | | 0.53 | 0.00 | 0.08 | 1.44 |
| | | 0.96 | 0.17 | 0.71 | 0.44 |
| | | 1.65 | 0.42 | 0.48 | 0.48 |
| | | 0.32 | 0.18 | 0.36 | 0.57 |
| | | 1.33 | 0.61 | 0.62 | 0.44 |
| | | — | — | — | — |
| | | — | — | — | — |
| | | — | — | — | — |
| | Avg. | 0.96 | 0.24 | 0.55 | 0.72 |
| | TH | 0.18 | 0.41 | 0.12 | 0.02 |
| | | 0.49 | 0.00 | 0.09 | 0.10 |
| | | 0.50 | 0.02 | 0.08 | 0.06 |
| | | 0.50 | 0.44 | 0.00 | 0.05 |
| | | 0.42 | 0.17 | 0.00 | 0.16 |
| | | 0.74 | 0.30 | 0.02 | 0.06 |
| | | — | — | — | — |
| | | — | — | — | — |
| | | — | — | — | — |
| | Avg. | 0.47 | 0.22 | 0.05 | 0.07 |
| | BS | 0.99 | 0.49 | 0.00 | 0.03 |
| | | 0.74 | 0.00 | 0.05 | 0.16 |
| | | 0.23 | 0.15 | 0.03 | 0.12 |
| | | 0.24 | 0.41 | 0.02 | 0.03 |
| | | 0.44 | 0.58 | 0.08 | 0.14 |
| | | 0.30 | 0.29 | 0.07 | 0.17 |
| | | — | — | — | — |
| | | — | — | — | — |
| | | — | — | — | — |
| | Avg. | 0.49 | 0.32 | 0.04 | 0.11 |
| | SC | 0.44 | 1.09 | 0.29 | 0.05 |
| | | 0.68 | 0.00 | 0.12 | 0.34 |
| | | 0.71 | 0.62 | 0.09 | 0.15 |
| | | 1.27 | 0.27 | 0.12 | 0.29 |
| | | 0.00 | 0.29 | 0.14 | 0.11 |
| | | 0.89 | 1.11 | 0.19 | 0.25 |
| | | — | — | — | — |
| | | — | | — | — |
| | | — | — | — | — |
| | Avg. | 0.67 | 0.56 | 0.16 | 0.20 |

PT3 expression was evident in all CNS tissue tested and limited expression was detected in the peripheral tissues of mice treated with each of the viral genomes. Higher PT3 expression (ng/mg or PT3/vg) was observed when mice were administered HL.T2A viral genomes (i.e. TAU_ITR114 (SYN.HL.T2A); TAU_ITR113 (GFAP.HL.T2A) regardless of the promoter used when compared to PT3 expression obtained with HL.F2A configuration (i.e. TAU_ITR106 (SYN.HL. F2A); TAU_ITR105 (GFAP.HL.F2A).

Comparing the PT3 expression in mice treated with TAU_ITR112 (Voy101.CAG.PT3.H.F.T2A.L) described in Table 163 with TAU_ITR113 (GFAP.HL.T2A) or TAU_ITR114 (SYN.HL.T2A), CAG.PT3.H.F.T2A.L resulted in significantly higher expression in the cortex, hippocampus and thalamus compared to GFAP.HL.T2A (p value <0.005 (cortex) and <0.0005 (hippocampus and thalamus). Expression of PT3 in cortex and hippocampus of mice treated with CAG.PT3.H.F.T2A.L was also higher in comparison to PT3 expression in mice treated with SYN.HL.T2A (p value <0.0005). In the thalamus, SYN.HL.T2A resulted in higher PT3 expression compared to GFAP.HL.T2A (p value <0.005).

PT3 expression in the CSF and serum were also measured (Table 170).

TABLE I70
PTS expression (PT3/vg) in serum and CSF

| | | TAU_ITR114 | TAU_ITR118 | TAU_ITR113 | TAU_ITR117 |
|---|---|---|---|---|---|
| | PBS | (SYN.HLT2A) | (SYN.LHT2A) | (GFAP.HLT2A) | (GFAP.LHT2A) |
| Serum | 0 | 1422.62 | 3588.88 | 27805.72 | 244053.65 |
| | 0 | 387.99 | 4257.08 | 126354.36 | 158211.62 |
| | 0 | 32720.22 | 4372.04 | 116784.02 | 280417.29 |
| | 0 | 10216.99 | 3078.75 | 164247.74 | 349567.81 |
| | 0 | 6078.46 | 3387.70 | 163730.42 | 175380.03 |
| | 0 | 19270.01 | 3064.38 | 249475.50 | 569538.00 |
| | 0 | — | — | 272754.71 | — |
| | 0 | — | — | 92470.18 | — |
| Avg. | 0 | 11682.71 | 3624.80 | 151702.83 | 296194.73 |
| CSF | 0 | 227.49 | 0 | 445.07 | 116.53 |
| | 0 | 315.66 | 158.17 | 109.68 | 120.10 |
| | 0 | 269.25 | 123.37 | 182.48 | 0.00 |
| | 0 | 383.49 | 216.18 | 223.62 | 135.56 |
| | 0 | 87.67 | 185.84 | 313.16 | 196.55 |
| | 0 | 416.69 | | 497.31 | 170.67 |
| | 0 | 336.56 | — | 249.55 | — |
| | 0 | 379.55 | — | 315.74 | — |
| | | 349.95 | — | — | — |
| Avg. | 0 | 307.37 | 136.71 | 292.08 | 123.23 |

| | TAU_ITR106 | TAU_ITR110 | TAU_ITR105 | TAU_ITR109 |
|---|---|---|---|---|
| | (SYN.HLF2A) | (SYN.LHF2A) | (GFAP.HLF2A) | (GFAP.LHF2A) |
| Serum | 12652.76 | 4884.64 | 34214.00 | 27938.64 |
| | 15906.61 | 308.06 | 20636.41 | 63613.08 |
| | 11261.80 | 4041.58 | 62015.72 | 56539.05 |
| | 15037.26 | 4808.57 | 39918.86 | 86166.33 |
| | 6120.22 | 1404.67 | 57718.05 | 89931.54 |
| | 6741.18 | 6895.28 | 24363.59 | 69774.34 |
| | — | — | — | — |
| | — | — | — | — |
| Avg. | 11286.64 | 3723.80 | 39811.11 | 65660.50 |
| CSF | 109.39 | 31.44 | 30.55 | 25.20 |
| | 215.29 | 0 | 19.54 | 63.57 |
| | 163.53 | 0 | 26.98 | 215.29 |
| | 203.69 | 64.76 | 25.20 | 59.71 |
| | 237.30 | 11.81 | | 54.05 |
| | 201.31 | 37.99 | | |
| | — | — | — | — |
| | — | — | — | — |
| | — | — | — | — |
| Avg. | 188.42 | 24.33 | 25.57 | 83.56 |

In T2A group, the serum of mice treated with viral genome configuration TAU_ITR117 (GFAP.LH. T2A) demonstrated a significantly higher PT3 expression compared to mice treated with TAU_ITR114 (SYN.HL.T2A); TAU_ITR118 (SYN.LH. T2A); or TAU_ITR113 (GFAP.HL.T2A) (p value <0.0005; 2 way ANOVA). PT3 expression in the serum of mice treated with TAU_ITR114 (SYN.HL.T2A) was also significantly higher than the expression in the serum of mice treated with TAU_ITR118 (SYN.LH. T2A); and significantly lower than the PT3 expression after administration of TAU_ITR113 (GFAP.HL.T2A) (p value <0.0005). No significant difference in PT3 expression was observed in mice treated with any of the F2A based genome configurations. The CSF of mice treated with TAU_ITR114 (SYN.HL.T2A) showed significantly higher PT3 expression compared to mice treated with TAU_ITR117 (GFAP.LH. T2A) (p value <0.0005) or TAU_ITR118 (SYN.LH. T2A) (p value <0.005). Similarly, PT3 expression in the CSF of mice treated with TAU_ITR113 (GFAP.HL.T2A) was higher than the expression in mice treated with TAU_ITR117 (GFAP.LH. T2A) or TAU_ITR118 (SYN.LH. T2A) (p value <0.005). In the F2A cohort, PT3 expression in the CSF was significantly greater in mice treated with TAU_ITR114 (SYN.HL.T2A) compared to mice treated with TAU_ITR118 (SYN.LH. T2A) (p value <0.005) or TAU_ITR113 (GFAP.HL.T2A) (p value <0.05). In general, in the T2A cohort, the PT3 expression in the CSF was higher in mice treated with HL configuration genomes compared to the LH genome configuration regardless of the promoter used.

The PT3 expression obtained using cell specific promoters in various genome configurations was compared with IPN002 expression in mice using identical promoters and genome configurations (see Table 108 and Table 111 in Example 8). Regardless of the cell specific promoter used, the HLT2A genome configuration resulted in higher anti tau antibody expression (ng/mg or ab/vg) in the CNS compared to the LHT2A or HLF2A genome configurations. Overall, higher IPN002 expression than PT3 expression (ab/vg) was obtained when using similar genome configurations. In both the PT3 study (instant example) and the IPN002 (Example 8), low levels of anti-tau antibody were observed in the peripheral tissues. In general, serum antibody levels were higher when the GFAP promoter was used than when the Syn promoter was used. Within the GFAP promoter based constructs, higher antibody expression was observed within the T2A group than the F2A group. In the CSF, no significant difference was observed in IPN002 study across all genome configurations analyzed within the T2A or F2A groups. However. HLT2A configuration resulted in higher IPN002 expression compared to HLF2A configuration or LHT2A configuration.

Taken together, these studies demonstrated that the mice treated with antibody constructs comprising the HLT2A genome configuration (heavy-linker-light) demonstrated comparatively more antibody expression (ab/vg), based on functional binding ELISA.

Anti-tau antibody distribution was evaluated using anti-IgG1 antibody-immunohistochemistry using DAB for detection. Broad distribution of PT3 expression in the brain was observed in all the tested viral genome configurations except the PBS group. Cell specific expression was measured in conjunction with anti-S100β immunohistochemistry using green chromogen (S100β is used an astrocytic cell marker). Promoter specific expression was observed in the neurons or astrocytes of mice treated with the corresponding promoters.

Comparing PT3 expression (ng/mg) among ubiquitous and cell specific promoters utilizing TAU_ITR111 (Voy101.CBA.PT3.H.F.T2A.L), TAU_ITR112 (Voy101.CAG.PT3.H.F.T2A.L), TAU_ITR113 (GFAP.HL.T2A), TAU_ITR114 (SYN.HL.T2A) constructs, it was noted that ubiquitous promoters produced higher PT3 expression in the CNS compared to cell-specific promoters. However, the PT3 levels in the CSF were comparable among all 4 promoters.

### Example 21. In Vivo Analysis of Constructs for Expression of Antibody Fragments

### IPN002 Fab Viral Genomes in Mice

To study biodistribution, tissue tropism and expression patterns of anti-tau antibody IPN002 fragments (Fab vs (Fab′)²) driven by CAG promoter in serum, CSF and/or CNS tissue of mice after intravenous delivery of AAV particles, viral genomes described in Table 171 were packaged into VOY101 capsids. AAV particles were formulated and provided intravenously at a dose of 1.4×10¹³ vg/kg into 2 month-old female C57Bl/6 mice. 28 days after administration, mice were sacrificed, perfused intracardially with PBS and CSF and/or tissue samples (hippocampus, cortex, thalamus, brain stem, spinal cord, olfactory bulb, CSF) were collected for analysis. Study design is shown in Table 171.

TABLE 171
Biodistribution of VOY101.IPN002 viral genomes (AAV genomes/cell)

| | | SEQ | | | | | | End |
|---|---|---|---|---|---|---|---|---|
| | TAU_ITR | ID | | Dose | Dose | | | of |
| AAV Particle | ID | NO: | μL | (vg/kg) | (vg) | Route | N | study |
| IPN002.(Fab\grave{ })₂.CAG.HL.T2 | TAU_ITR126 | 2176 | 100 | 1.4 × 10¹³ | 2.8 × 10¹¹ | IV | 6 | Day 28 |
| IPN002.Fab_ext.CAG.HL.T2 | TAU_ITR128 | 2178 | 100 | 1.4 × 10¹³ | 2.8 × 10¹¹ | IV | 6 | Day 28 |
| IPN002.Fab_ext.CAG.LH.T2 | TAU_ITR130 | 2180 | 100 | 1.4 × 10¹³ | 2.8 × 10¹¹ | IV | 6 | Day 28 |
| PBS | — | — | 100 | — | — | IV | 4 | D 28 |

VG/DC (vector genome/diploid cell) was quantified for a subset of tissues, such as for brain, and CSF by droplet digital PCR. Antibody levels in the hippocampus, cortex, olfactory bulb, and CSF were quantified using ePHF ELISA, as described above. Vector genome biodistribution quantification (VG/DC) in brain and spinal cord are shown in Table 172 and the antibody level measurements are shown in Table 173 and Table 174. Significance was analyzed using one-way ANOVA-Tukey's multiple comparison test.

TABLE 172
Biodistribution of VOY101.IPN002 viral genomes (AAV genomes/cell)

| AAV | | | Olfactory | | | Spinal |
|---|---|---|---|---|---|---|
| Partide | Hippocampus | Cortex | bulb | Thalamus | Brainstem | Cord |
| TAU_ITR126 | 15.82 | 18.60 | 9.17 | 62.46 | 38.13 | 21.96 |
| (IPN002.(Fab\grave{ })₂.CAG.HL.T2) | 20.55 | 27.61 | 21.94 | 80.46 | 52.24 | 20.40 |
| | 30.65 | 13.52 | 20.70 | 55.02 | 41.81 | 29.69 |
| | 28.30 | 30.93 | 31.83 | 62.82 | 105.57 | 83.17 |
| | 15.14 | 20.04 | 27.89 | 55.62 | 62.12 | 45.10 |
| | 13.35 | 14.19 | 25.05 | 39.21 | 56.85 | 32.47 |
| Average | 20.63 | 20.82 | 22.76 | 59.27 | 59.45 | 38.80 |
| TAU_ITR128 | 19.17 | 22.18 | 21.60 | 54.91 | 39.88 | 23.48 |
| (IPN002.Fab_ext.CAG.HL.T2) | 11.66 | 18.18 | 19.06 | 24.56 | 24.39 | 25.99 |
| | 14.09 | 25.64 | 48.77 | 84.41 | 64.66 | 4.93 |
| | 8.26 | 14.96 | 23.06 | 28.80 | 28.00 | 17.52 |
| | 2.45 | 3.36 | 2.90 | 8.50 | 6.33 | 38.29 |
| | 20.22 | 39.86 | 30.62 | 69.61 | 78.87 | 36.96 |
| Average | 12.64 | 20.70 | 24.33 | 45.13 | 40.36 | 24.53 |
| TAU_ITR130 | 17.16 | 23.89 | 20.93 | 45.81 | 57.77 | 31.99 |
| (IPN002.Fab_ext.CAG.LH.T2) | 21.36 | 51.64 | 21.64 | 59.06 | 77.14 | 53.97 |
| | 17.26 | 33.18 | 20.10 | 55.46 | 50.30 | 33.16 |
| | 24.74 | 25.15 | 27.57 | 32.72 | 64.24 | 30.44 |
| | 22.43 | 28.26 | 69.07 | 86.67 | 135.44 | 58.60 |
| | 59.46 | 27.90 | — | 20.49 | 39.21 | 20.51 |
| Average | 27.07 | 31.67 | 31.86 | 50.04 | 70.69 | 38.11 |
| PBS | — | 0.01 | 0.00 | 0.00 | 0.0 | 0.02 |

Among the Fab constructs tested, TAU_ITR130 (IPN002.Fab_ext.CAG.LH.T2) showed higher expression in all tissue types tested when compared to TAU_ITR128 (IPN002.Fab_ext.CAG.HL.T2). Among the (Fab′)2 based constructs and the Fab constructs tested, the (Fab′)2 construct TAU_ITR126 (IPN002.(Fab′)2.CAG.HL.T2) showed highest expression.

TABLE 173
IPN002 fragment expression in CNS

| | Hippocampus | | Cortex | | Olfactory bulb | |
|---|---|---|---|---|---|---|
| | ng/mg | scFv/ | ng/mg | | ng/mg | |
| Construct | protein | Vg | protein | scFv/Vg | protein | seFv/Vg |
| TAU_ITR126 | 5.18 | 6.33 | 16.17 | 0.87 | 14.00 | 1.53 |
| (IPN002.(Fab\grave{ })₂.CAG.HL.T2) | 14.25 | 0.69 | 22.42 | 0.81 | 17.24 | 0.79 |
| | 7.68 | 0.25 | 11.31 | 0.84 | 8.22 | 0.40 |
| | 14.18 | 0.50 | 23.90 | 0.77 | 23.72 | 0.75 |
| | 9.42 | 0.62 | 20.19 | 1.01 | 20.91 | 0.75 |
| | 7.34 | 0.55 | 20.63 | 1.45 | 19.72 | 0.79 |
| Average | 9.68 | 0.49 | 19.10 | 0.96 | 17.30 | 0.83 |
| TAU_ITR128 | 38.48 | 2.01 | 48.38 | 2.18 | 44.25 | 2.05 |
| (IPN002.Fab_ext.CAG.HL.T2) | 15.52 | 1.33 | 51.81 | 2.85 | 28.63 | 1.50 |
| | 31.54 | 2.24 | 55.66 | 2.17 | 38.50 | 0.79 |
| | 19.38 | 2.35 | 37.28 | 2.49 | 30.98 | 1.34 |
| | 2.86 | 1.87 | 3.39 | 1.32 | 47.55 | 1.55 |
| | 37.92 | — | 52.48 | — | — | |
| Average | 24.28 | 1.96 | 41.50 | 2.20 | 37.98 | 1.45 |
| TAU_ITR130 | 24.52 | 1.43 | 37.72 | 1.58 | 22.80 | 1.09 |
| (IPN002.Fab_ext.CAG.LH.T2) | 9.73 | 0.46 | 43.89 | 0.85 | 21.23 | 0.98 |
| | 9.35 | 0.77 | 25.22 | 0.72 | 10.84 | 0.94 |
| | 13.29 | 0.59 | 23.90 | 1.11 | 18.90 | 0.27 |
| | 12.81 | 0.30 | 22.37 | 1.79 | 18.53 | — |
| | 13.26 | — | 31.24 | — | 22.92 | — |
| | 17.86 | — | 49.90 | — | — | — |
| Average | 14.40 | 0.71 | 33.46 | 1.21 | 19.20 | 0.82 |
| Vehicle | 0.00 | 0.00 | 0.00 | 0.00 | 0.00 | 0.00 |
| (PBS) | | | | | | |

TABLE 174
IPN002 Fab or (Fab′)₂ expression in CSF

| | ng/mg | pM |
|---|---|---|
| | (Fab′)2 or | (Fab′)2 or |
| Construct | Fab per ml | Fab |
| TAU_ITR126 | 43.63 | 392.54 |
| (IPN002.(Fab′)₂.CAG.HL.T2) | 46.48 | 418.18 |
| | 23.94 | 215.38 |
| | 34.56 | 310.96 |
| | — | — |
| | — | — |
| Average | 37.15 | 334.27 |
| TAU_ITR128 | 308.03 | 6165.939 |
| (IPN002.Fab_ext.CAG.HL.T2) | 295.16 | 5908.297 |
| | 343.59 | 6877.729 |
| | 208.77 | 4179.039 |
| Average | 288.89 | 5782.751 |
| TAU_ITR130 | 189.14 | 3786.026 |
| (IPN002.Fab_ext.CAG.LH.T2) | 192.19 | 3847.162 |
| | 129.36 | 2589.520 |
| | 211.61 | 4235.808 |
| | 172.12 | 3445.415 |
| | 212.26 | 4248.908 |
| Average | 184.45 | 3692.140 |
| Vehicle (PBS) | 0.00 | 0.00 |

Among the Fab constructs. HL based construct TAU_ITR128 (IPN002.Fab_ext.CAG.HL.T2), appeared to have higher expression compared to TAU_ITR130 (IPN002.Fab_ext.CAG.LH.T2).

Immunohistochemistry using anti-Kappa antibody was performed on brain tissue of mice treated with an IV bolus injection of ssVOY101.IPN002 AAV particles comprising viral genomes encoding (Fab′)₂ or Fab, driven by CAG promoters 28 days prior. Broad distribution of IPN002 (Fab′)₂ or Fab antibody delivered by VOY101 using CAG promoter with the configurations described in Table 171 was observed both in the CNS and in the spinal cord. Stronger staining in multiple CNS regions was detected with TAU_ITR128 (IPN002.Fab_ext.CAG.HL.T2) as compared to the other constructs listed in Table 171.

Localization studies showed that all three constructs listed in Table 171 were distributed to both neurons and astrocytes.

### PT3 Fab Viral Genomes in Mice

To study the biodistribution, tissue tropism and expression of anti-tau antibody PT3 fragments (Fab and (Fab)₂) in serum, CSF and/or CNS tissue of mice after intravenous delivery of AAV particles, viral genomes described in Table 175 were packaged into VOY101 capsids. AAV particles were formulated and provided intravenously at a dose of 1.4×10¹³ vg/kg to 2 month-old female C57Bl/6 mice. Twenty-eight days after administration, mice were sacrificed, perfused intracardially with PBS and CSF and/or tissue samples (hippocampus, cortex, thalamus, brain stem, spinal cord, olfactory bulb, CSF) were collected for analysis. Study design is shown in Table 175.

TABLE 175
PT3 Fab/F(ab\grave{ })₂ IV study design

| | | SEQ | | | | | | End |
|---|---|---|---|---|---|---|---|---|
| | TAU_ITR | ID | | Dose | ~Dose | | | of |
| Construct Configuration | ID | NO: | μl | VG/kg | Vg | Route | N | study |
| CAG PT3_Fab H.F.T2A.L | TAU_ITR180 | 2230 | 100 | 1.4E13 | 2.8E11 | IV | 6 | D 28 |
| SYN.PT3_Fab H.F.T2A.L | TAU_ITR188 | 2260 | 100 | 1.4E13 | 2.8E11 | IV | 6 | D 28 |
| GFAP.PT3_Fab H.F.T2A.L | TAU_ITR189 | 2261 | 100 | 1.4E13 | 2.8E11 | IV | 6 | D 28 |
| CAG PT3_F(ab′)₂ | TAU_ITR190 | 2262 | 100 | 1.4E13 | 2.8E11 | IV | 6 | D 28 |
| H.hIgG3_3Hinge.F.T2A.L | | | | | | | | |
| SYN.PT3_F(ab′)2 | TAU_ITR191 | 2263 | 100 | 1.4E13 | 2.8E11 | IV | 6 | D 28 |
| H.hIgG3_3Hinge.F.T2A.L | | | | | | | | |
| GFAP.PT3_F(ab′)₂ | TAU_ITR192 | 2264 | 100 | 1.4E13 | 2.8E11 | IV | 6 | D 28 |
| H.hIgG3_3Hinge.F.T2A.L | | | | | | | | |
| CAG PT3_F(ab′)₂ | TAU_ITR193 | 2265 | 100 | 1.4E13 | 2.8E11 | IV | 6 | D 28 |
| H.hIgG3_3Hinge.F.F2A.L | | | | | | | | |
| SYN PT3_F(ab′)₂ | TAU_ITR194 | 2266 | 100 | 1.4E13 | 2.8E11 | IV | 6 | D 28 |
| H.hIgG3_3Hinge.F.F2A.L | | | | | | | | |
| GFAP PT3_F(ab′)₂ | TAU_ITR195 | 2267 | 100 | 1.4E13 | 2.8E11 | IV | 6 | D 28 |
| H.hIgG3_3Hinge.F.F2A.L | | | | | | | | |
| PBS | — | — | 100 | | — | IV | 8 | D 28 |

Vector genome was quantified for a subset of tissues, such as for brain, spinal cord, CSF, serum, and liver by droplet digital PCR and normalized per diploid cell (VG/DC) using the endogenous mouse transferrin receptor C gene (TFRC). Antibody levels in the tissues were quantified using ePHF ELISA, as described above. For these experiments, an anti-kappa-HRP was used as the detection antibody in the ePHF ELISA and a recombinant PT3 Fab or F(ab′)2 was used as a standard. Vector genome biodistribution quantification (VG/DC) in brain and spinal cord are shown in Table 176 and the antibody level measurements are shown in Table 177 and Table 178. Significance was analyzed using one-way ANOVA-Tukey's multiple comparison test. The results were also compared to the data obtained by transducing rodents with scFv constructs described in Table 153. Table 154. Table 155, and Table 156.

TABLE 176
Biodistribution of VOY101.PT3 viral
genomes (AAV genomes/cell)

| Construct Configuration | | | Olfactory | Spinal |
|---|---|---|---|---|
| (TAU_ITR ID) | Cortex | Hippocampus | Bulb | Cord |
| Vehicle | 0.00 | 0.00 | 0.00 | 0.00 |
| GFAP.F(ab′)₂.HL.T2A | 59.66 | 96.407 | 93.93 | 175.95 |
| (TAU_ITR192) | 25.84 | 16.215 | 58.22 | 90.17 |
| | 35.97 | 34.999 | 41.80 | 77.01 |
| | 13.17 | 17.486 | 33.98 | 30.39 |
| | 36.06 | 65.657 | 82.67 | 133.80 |
| | 36.49 | 39.704 | 57.63 | 76.65 |
| Average | 34.53 | 45.08 | 61.37 | 97.33 |
| SYN.F(ab′)₂.HL.T2A | 25.76 | 29.356 | 33.32 | 62.15 |
| (TAU_ITR191) | 33.72 | 65.259 | 72.73 | 35.69 |
| | 35.29 | 31.863 | 45.38 | 114.84 |
| | 31.71 | 40.863 | 72.96 | 123.35 |
| | 34.91 | 44.189 | 51.50 | 97.39 |
| | 41.11 | 39.333 | 56.87 | 67.66 |
| Average | 33.75 | 41.81 | 55.46 | 83.51 |
| CAG.F(ab′)₂.HL.T2A | 18.00 | 16.861 | 16.48 | 86.92 |
| (TAU_ITR190) | 15.22 | 14.334 | 15.21 | 81.93 |
| | 9.27 | 15.698 | 31.06 | 52.76 |
| | 11.63 | 15.610 | 15.22 | 11.52 |
| | 13.11 | 6.386 | 15.25 | 18.41 |
| | 11.39 | 8.523 | 13.57 | 19.78 |
| Average | 13.10 | 12.90 | 17.80 | 45.22 |
| CAG.F(ab′)₂.HL.F2A | 28.54 | 32.441 | 73.48 | 110.55 |
| (TAU_ITR193) | 23.40 | 20.576 | 52.51 | 103.39 |
| | 36.16 | 37.218 | 69.30 | 127.66 |
| | 34.23 | 29.131 | 37.18 | 66.41 |
| | 27.94 | 33.222 | 57.25 | 73.27 |
| | 44.03 | 34.568 | 50.42 | 106.80 |
| Average | 32.38 | 31.19 | 56.69 | 98.01 |
| SYN.Fab.HL.T2A | 20.06 | 11.107 | 27.75 | 35.30 |
| (TAU_ITR188) | 30.05 | 28.200 | 46.34 | 122.48 |
| | 60.13 | 82.673 | 63.78 | 30.90 |
| | 57.01 | 59.839 | 81.80 | 307.92 |
| | 34.60 | 62.331 | 67.39 | 112.09 |
| | 44.07 | 25.380 | 151.00 | 160.08 |
| Average | 40.99 | 44.92 | 73.01 | 128.13 |
| CAG.Fab.HL.T2A | 38.22 | 110.961 | 42.12 | 35.75 |
| (TAU_ITR180) | 15.13 | 20.688 | 25.32 | 47.77 |
| | 16.55 | 18.755 | 51.72 | 61.31 |
| | 17.98 | 10.043 | 31.93 | 50.11 |
| | 22.51 | 24.999 | 26.60 | 55.10 |
| | 30.71 | 50.367 | 45.16 | 91.20 |
| | 12.00 | 16.332 | 15.08 | 53.24 |
| Average | 21.87 | 36.02 | 33.99 | 56.36 |
| GFAP.F(ab′)₂.HL.F2A | 21.62 | 20.987 | 35.76 | 40.35 |
| (TAU_ITR195) | 27.14 | 108.730 | 66.79 | 113.01 |
| | 39.12 | 66.121 | 168.32 | 176.09 |
| | 22.65 | 48.241 | 34.40 | 45.45 |
| | 42.00 | 77.394 | 47.39 | 82.56 |
| | 19.74 | 46.927 | 63.92 | 81.14 |
| Average | 28.71 | 61.40 | 69.43 | 89.77 |
| SYN.F(ab′)₂.HL.F2A | 21.77 | 61.32 | 47.43 | 78.25 |
| (TAU_ITR194) | 38.78 | 73.78 | 49.62 | 143.28 |
| | 36.45 | 42.48 | 79.82 | 87.14 |
| | 57.30 | 102.33 | 136.96 | 155.97 |
| | 27.85 | 85.72 | 97.86 | 116.69 |
| | 24.01 | 55.63 | 54.33 | 51.00 |
| Average | 34.36 | 70.21 | 77.67 | 105.39 |
| GFAP.Fab.HL.T2A | 50.64 | 56.364 | 63.86 | 127.32 |
| (TAU_ITR189) | 51.51 | 58.083 | 108.95 | 60.53 |
| | 49.01 | 79.911 | 130.12 | 165.26 |
| | 40.72 | 53.395 | 125.42 | 97.73 |
| | 43.82 | 55.946 | 66.42 | 109.34 |
| | 29.67 | 51.120 | 61.83 | 73.39 |
| Average | 44.23 | 59.14 | 92.77 | 105.59 |

As shown in Table 176, among the constructs with a T2A cleavage site, constructs with GFAP and SYN promoters generally showed higher AAV biodistribution than constructs operably linked to a CAG promoter. For transgenes with F2A peptide cleavage sites, this trend is only observed in hippocampal tissue. Delivery of Fab constructs (TAU_ITR180, TAU_ITR189 and TAU_ITR188) yielded the highest AAV genomes per cell, as compared to F(ab′)2 or scFv constructs comprising the same promoter (e.g., CAG.Fab resulted in more AAV genomes per cell than CAG. F(ab′)2 or CAG.scFv).

Using One-way ANOVA-Tukey's multiple comparison test, statistical analyses were performed for AAV distribution of each form of fragment (Fab, F(ab′)2, and scFv) driven by each promoter and in each tissue. Assessment of biodistribution in the cortex yielded the following statistically significant relationships. In comparing constructs encoding Fab fragments, administration of TAU_ITR189 (GFAP.Fab) and TAU_ITR188 (SYN.Fab) resulted in significantly more AAV genomes per cell as compared to TAU_ITR180 (CAG.Fab) (p values <0.0005 and <0.005 respectively). For the F(ab′)2 fragment group, a similar pattern was evident, with delivery of TAU_ITR192 (GFAP. F(ab′)₂) generating significantly more AAV genomes per cell in the cortex than delivery of TAU_ITR190 (CAG. F(ab′)2) (p value <0.005). In comparing biodistribution of constructs encoding scFv fragments (TAU_ITR162. TAU_ITR165, and TAU_ITR168; Table 154), delivery of TAU_ITR165 (GFAP.scFv) resulted in greater biodistribution to the cortex than delivery of TAU_ITR162 (CAG.scFv) (p value <0.05). Comparisons across fragment type with expression driven by the same promoter showed significantly greater biodistribution to the cortex and the hippocampus following delivery of TAU_ITR188 (SYN.Fab) than TAU_ITR168 (SYN.scFv) (p value <0.005) and significantly greater biodistribution to cells of the hippocampus subsequent to delivery of TAU_ITR192 (GFAP. F(ab′)₂) than TAU_ITR165 (GFAP.scFv) (p value <0.05).

Antibody levels in the various tissues were quantified using ePHF ELISA, as described above, but using an anti-Kappa-HRP as a detection antibody The antibody level measurements in the cortex, hippocampus, olfactory bulb, and spinal cord are shown in Table 177 and Table 178. Significance was analyzed using one-way ANOVA-Tukey's multiple comparison test.

TABLE 177
PT3 Fab/F(ab′)₂ expression (ng/mg)

| Construct | | | Olfactory | Spinal |
|---|---|---|---|---|
| (TAU_ITR ID) | Cortex | Hippocampus | Bulb | Cord |
| Vehicle | 0.00 | 1.69 | 0.00 | 0.00 |
| CAG.F(ab′)₂.HL.F2A | 7.30 | 0.00 | 25.66 | 67.08 |
| (TAU_ITR.193) | 12.99 | 7.50 | 26.31 | 76.64 |
| | 15.36 | 10.92 | 43.98 | 65.61 |
| | 8.45 | 6.57 | 37.95 | 42.91 |
| | 22.62 | 11.19 | 36.26 | 95.39 |
| | 20.20 | 14.09 | 66.37 | 85.58 |
| Average | 14.49 | 8.38 | 39.42 | 72.20 |
| SYN.Fab.HL.T2A | 0.51 | −0.22 | 0.00 | 0.00 |
| (TAU_ITR188) | 10.97 | 10.13 | 32.01 | 234.75 |
| | 6.88 | 9.58 | 36.81 | 182.59 |
| | 28.19 | 36.04 | 70.50 | 363.45 |
| | 7.05 | 10.02 | 25.76 | 185.08 |
| | 16.67 | 32.51 | 90.01 | 391.81 |
| Average | 11.71 | 16.34 | 42.51 | 226.28 |
| CAG.Fab.HL.T2A | 213.95 | 152.15 | 246.17 | 400.88 |
| (TAU_ITR180) | 37.26 | 30.67 | 184.38 | 180.63 |
| | 106.69 | 71.19 | 196.59 | 297.40 |
| | 51.67 | 45.98 | 252.33 | 374.69 |
| | 42.30 | 30.46 | 158.28 | 141.95 |
| | 128.16 | 92.23 | 261.18 | 284.72 |
| | 24.33 | 15.50 | 129.56 | 139.25 |
| Average | 86.34 | 62.60 | 204.07 | 259.93 |
| GFAP.F(ab′)₂.HL.F2A | 0.00 | 0.00 | 0.00 | 0.00 |
| (TAU_ITR195) | 0.00 | 0.00 | 18.25 | 9.85 |
| | 3.18 | 0.00 | 22.67 | 11.22 |
| | 0.00 | 0.00 | 13.02 | 12.81 |
| | 3.87 | 0.00 | 47.61 | 29.23 |
| | 0.00 | 0.00 | 14.03 | 9.43 |
| Average | 1.18 | 0.00 | 19.26 | 12.09 |
| SYN.F(ab′)₂.HL.F2A | 0.00 | 0.00 | 0.00 | 45.63 |
| (TAU_ITR194) | 0.00 | 0.00 | 0.00 | 37.95 |
| | 0.00 | 0.00 | 0.00 | 67.24 |
| | 0.00 | 0.00 | 9.28 | 83.39 |
| | 0.00 | 0.00 | 9.78 | 76.17 |
| | 0.00 | 0.00 | — | 62.03 |
| Average | 0.00 | 0.00 | 3.81 | 62.07 |
| GFAP.Fab.HL.T2A | 17.63 | 12.07 | 123.89 | 38.49 |
| (TAU_ITR189) | 42.89 | 14.45 | 221.26 | 58.23 |
| | 29.03 | 22.36 | 262.50 | 112.04 |
| | 14.59 | 12.94 | 119.80 | 74.22 |
| | 27.13 | 10.66 | 154.62 | 90.98 |
| | 48.47 | 15.16 | 234.40 | 108.85 |
| Average | 29.96 | 14.61 | 186.08 | 80.47 |
| GFAP.F(ab′)₂.HL.T2A | 3.87 | 0.00 | 48.19 | 57.72 |
| (TAU_ITR192) | 0.00 | 0.00 | 28.06 | 22.84 |
| | 6.17 | 0.00 | 43.30 | 38.07 |
| | 0.00 | 0.00 | 0.00 | 7.36 |
| | 5.28 | 0.00 | 0.00 | 46.19 |
| | 4.27 | 0.00 | 24.68 | 52.93 |
| Average | 3.27 | 0.00 | 24.04 | 37.52 |
| SYN.F(ab′)₂.HL.T2A | 2.70 | 0.00 | 9.40 | 128.50 |
| (TAU_ITR191) | 0.00 | 0.00 | 11.47 | 87.30 |
| | 3.10 | 0.00 | 14.70 | 124.64 |
| | 3.29 | 0.00 | 12.12 | 229.45 |
| | 3.55 | 0.00 | 16.95 | 157.90 |
| | 2.97 | 0.00 | 11.61 | 185.24 |
| Average | 2.60 | 0.00 | 12.71 | 152.17 |
| CAG.F(ab′)₂.HL.T2A | 2.66 | 0.00 | 13.09 | 53.44 |
| (TAU_ITR190) | 3.41 | 6.28 | 12.53 | 61.70 |
| | 3.74 | 0.00 | 8.86 | 73.11 |
| | 2.61 | 0.00 | 10.77 | 48.82 |
| | 3.67 | 0.00 | 22.19 | 92.71 |
| | 3.81 | 0.00 | 13.06 | 139.20 |
| Average | 3.32 | 1.05 | 13.42 | 78.16 |

As shown in Table 177, high levels of PT3 Fab were observed in the hippocampus, cortex and olfactory bulb of the mice dosed with Fab constructs as compared to mice dosed with F(ab′)₂ constructs. In contrast to the vector genome quantification data, use of a CAG promoter appeared to drive higher expression when compared to constructs comprising a cell specific promoter. High levels of PT3 Fab were also observed in the olfactory bulb of the mice dosed with Fab constructs as compared to those dosed with F(ab′)2 vectors. PT3 expression (ng/mg) was significantly higher (p value <0.0005) in the cortex of rodents transduced with CAG.Fab.HL.T2A (TAU_ITR180) when compared to the expression in the cortex of rodents transduced with SYN.Fab.HL.T2A (TAU_ITR188) or ssAAV CAG PT3 VL G4S-3 VH HA rBGpA (TAU_ITR162). PT3 expression (ng/mg) was significantly higher (p value <0.005) in the cortex of rodents transduced with CAG.Fab.HL.T2A (TAU_ITR180) when compared to the expression in the cortex of rodents transduced with GFAP.Fab.HL.T2A (TAU_ITR189) or CAG.F(ab′)2.HL.T2A (TAU_ITR190). In the hippocampus, PT3 expression (ng/mg) was significantly higher, p value <0.0005, in the rodents transduced with CAG.Fab.HL.T2A (TAU_ITR180) compared to SYN.Fab.HL.T2A (TAU_ITR188). GFAP.Fab.HL.T2A (TAU_ITR189), or ssAAV CAG PT3 VL G4S-3 VH HA rBGpA (TAU_ITR162). PT3 expression (ng/mg) was also significantly higher (p value <0.005) in the hippocampus of rodents transduced with CAG.Fab.HL.T2A (TAU_ITR180) compared to rodents transduced with CAG.F(ab′)2.HL.T2A (TAU_ITR190).

In spinal cord, PT3 expression levels were similar when driven by CAG or SYN promoter, regardless of whether the PT3 fragment was Fab or F(ab′)₂.

TABLE 178
Normalized PT3 Fab/F (ab′)2 expression (PT3/vector genome)

| Construct | | | Olfactory | Spinal |
|---|---|---|---|---|
| (TAU_ITR ID) | Cortex | Hippocampus | Bulb | cord |
| Vehicle | 0 | 0 | 0 | 0 |
| CAG.F(ab′)₂.HL.F2A | 0.26 | 0.00 | 0.35 | 0.61 |
| (TAU_ITR193) | 0.56 | 0.36 | 0.50 | 0.74 |
| | 0.42 | 0.29 | 0.63 | 0.51 |
| | 0.25 | 0.23 | 1.02 | 0.65 |
| | 0.81 | 0.34 | 0.63 | 1.30 |
| | 0.46 | 0.41 | 1.32 | 0.80 |
| Average | 0.46 | 0.27 | 0.74 | 0.77 |
| SYN.Fab.HL.T2A | 0.03 | 0.00 | 0.00 | 0.00 |
| (TAU_ITR188) | 0.37 | 0.36 | 0.69 | 1.92 |
| | 0.11 | 0.12 | 0.58 | 5.91 |
| | 0.49 | 0.60 | 0.86 | 1.18 |
| | 0.20 | 0.16 | 0.38 | 1.65 |
| | 0.38 | 1.28 | 0.60 | 2.45 |
| Average | 0.26 | 0.42 | 0.52 | 2.18 |
| CAG.Fab.HL.T2A | 5.60 | 1.37 | 5.84 | 11.21 |
| (TAU_ITR180) | 2.46 | 1.48 | 7.28 | 3.78 |
| | 6.45 | 3.80 | 3.80 | 4.85 |
| | 2.87 | 4.58 | 7.90 | 7.48 |
| | 1.88 | 1.22 | 5.95 | 2.58 |
| | 4.17 | 1.83 | 5.78 | 3.12 |
| | 20.3 | 0.95 | 8.59 | 2.62 |
| Average | 3.64 | 2.18 | 6.45 | 5.09 |
| GFAP.F(ab′)₂.HL.F2A | 0.00 | 0 | 0.00 | 0.00 |
| (TAU_ITR195) | 0.00 | 0 | 0.27 | 0.09 |
| | 0.08 | 0 | 0.13 | 0.06 |
| | 0.00 | 0 | 0.38 | 0.28 |
| | 0.09 | 0 | 1.00 | 0.35 |
| | 0.00 | 0 | 0.22 | 0.12 |
| Average | 0.03 | 0.00 | 0.34 | 0.15 |
| | 0.00 | 0 | 0.00 | 0.58 |
| | 0.00 | 0 | 0.00 | 0.26 |
| SYN.F(ab′)₂.HL.F2A | 0.00 | 0 | 0.00 | 0.77 |
| (TAU_ITR194) | 0.00 | 0 | 0.07 | 0.53 |
| | 0.00 | 0 | 0.10 | 0.65 |
| | 0.00 | 0 | 0.00 | 1.22 |
| Average | 0.00 | 0.00 | 0.03 | 0.67 |
| GFAP.Fab.HL.T2A | 0.35 | 0.21 | 1.94 | 0.30 |
| (TAU_ITR189) | 0.83 | 0.25 | 2.03 | 0.96 |
| | 0.59 | 0.28 | 2.02 | 0.68 |
| | 0.36 | 0.24 | 0.96 | 0.76 |
| | 0.62 | 0.19 | 2.33 | 0.83 |
| | 1.63 | 0.30 | 3.79 | 1.48 |
| Average | 0.73 | 0.25 | 2.18 | 0.84 |
| GFAP.F(ab′)₂.HL.T2A | 0.06 | 0 | 0.51 | 0.33 |
| (TAU_ITR192) | 0.00 | 0 | 0.48 | 0.25 |
| | 0.17 | 0 | 1.04 | 0.49 |
| | 0.00 | 0 | 0.00 | 0.24 |
| | 0.15 | 0 | 0.00 | 0.35 |
| | 0.12 | 0 | 0.43 | 0.69 |
| Average | 0.08 | 0.00 | 0.41 | 0.39 |
| SYN.F(ab′)₂.HL.T2A | 0.10 | 0 | 0.28 | 2.07 |
| (TAU_ITR191) | 0.00 | 0 | 0.16 | 2.45 |
| | 0.09 | 0 | 0.32 | 1.09 |
| | 0.10 | 0 | 0.17 | 1.86 |
| | 0.10 | 0 | 0.33 | 1.62 |
| | 0.07 | 0 | 0.20 | 2.74 |
| Average | 0.08 | 0.00 | 0.24 | 1.97 |
| CAG.F(ab′)₂.HL.T2A | 0.15 | 0.00 | 0.79 | 0.61 |
| (TAU_ITR190) | 0.22 | 0.44 | 0.82 | 0.75 |
| | 0.40 | 0.00 | 0.29 | 1.39 |
| | 0.22 | 0.00 | 0.71 | 4.24 |
| | 0.28 | 0.00 | 1.45 | 5.03 |
| | 0.33 | 0.00 | 0.96 | 7.04 |
| Average | 0.27 | 0.07 | 0.84 | 3.18 |

As shown in Table 178, inclusion of the CAG promoter resulted in higher PT3 expression in TAU_ITR180 (CAG PT3_Fab H.F.T2A.L), TAU_ITR190 (CAG PT3_F(ab′)2 H.hIgG3_3Hinge.F.T2A.L), TAU_ITR193 (CAG PT3_F(ab′)2 H.hIgG3_3Hinge.F.F2A.L) regardless of whether Fab or F(ab′)₂ antibody fragments were delivered to the cortex, hippocampus and olfactory bulb. In the spinal cord, CAG promoter appeared to drive PT3 expression in TAU_ITR180 (CAG PT3_Fab H.F.T2A.L), TAU_ITR190 (CAG PT3_F(ab′)2 H.hIgG3_3Hinge.F.T2A.L), TAU_ITR193 (CAG PT3_F(ab′)2 H.hIgG3_3Hinge.F.F2A.L) at levels similar to that seen with the SYN promoter constructs TAU_ITR188 (SYN.PT3_Fab H.F.T2A.L), TAU_ITR191 (SYN.PT3_F(ab′)2 H.hIgG3_3Hinge.F.T2A.L), TAU_ITR194 (SYN PT3_F(ab′)2 H.hIgG3_3Hinge.F.F2A.L). Comparing Fab and F(ab′)2 constructs operably linked to the same promoter, it was noted that PT3 Fab constructs (TAU_ITR180, TAU_ITR188, TAU_ITR189) resulted in higher expression per vector genome as compared to PT3 F(ab′)2 constructs (TAU_ITR190, TAU_ITR191, TAU_ITR192, TAU_ITR193, TAU_ITR194, TAU_ITR195).

Normalization of antibody fragments level showed a highest average level of expression per vector genome in the group dosed with CAG-Fab (TAU_ITR180). The statistical significance of the data in Table 178 was analyzed using one-way ANOVA-Tukey's multiple comparison test. Normalized PT3 expression (ab/vg) was significantly higher (p<0.0005) in the cortex of the former of the following pairs of comparison compared to the latter, (a) CAG PT3_Fab H.F.T2A.L (TAU_ITR180) compared to CAG PT3_F(ab′)2 H.hIgG3_3Hinge.F.T2A.L (TAU_ITR190), GFAP.PT3_Fab H.F.T2A. L (TAU_ITR189), or SYN.PT3_Fab H.F.T2A.L (TAU_ITR188) (b) ssAAV CAG PT3 VL G4S-3 VH HA rBGpA (TAU_ITR162) compared to ssAAV SYN PT3 VL G4S-3 VH HA rBGpA (TAU_ITR168) or ssAAV GFAP PT3 VL G4S-3 VH HA rBGpA (TAU_ITR165) or CAG PT3_F(ab′)2 H.hIgG3_3Hinge.F.T2A.L (TAU_ITR190). In the hippocampus, normalized PT3 expression (ab/vg) was significantly higher (p value <0.0005) in CAG PT3_Fab H.F.T2A.L (TAU_ITR180) compared to CAG PT3_F(ab′)2 H.F.T2A.L (TAU_ITR190), ssAAV CAG PT3 VL G4S-3 VH HA rBGpA (TAU_ITR162), SYN.PT3_Fab H.F.T2A.L (TAU_ITR188), or GFAP.PT3_Fab H.F.T2A.L (TAU_ITR189).

Antibody levels in the CSF, serum and liver tissues were also quantified using ePHF ELISA, as described above and as shown in Table 179. Also included in the table are vector genome quantification of the biodistribution to the liver (VG/DC).

TABLE 179
PT3 expression in CSF serum and liver

| | CSF | CSF | Serum | Serum | Liver | Liver |
|---|---|---|---|---|---|---|
| Description | (ng/ml) | (nM) | (ng/ml) | (nM) | CVG/DC) | (ng/mg) |
| CAG.F(ab′)₂.HL.F2A | 95.4 | 0.87 | 306.50 | 2.79 | 3.07 | 21.30 |
| (TAU_ITR193) | 101.55 | 0.92 | 6248.75 | 56.81 | 3.93 | 20.32 |
| | 70.25 | 0.64 | 5193.75 | 47.22 | 4.75 | 36.75 |
| | 46.45 | 0.42 | 5420.75 | 49.28 | 3.11 | 25.63 |
| | 97.15 | 0.88 | 12421.50 | 112.92 | 5.80 | 34.15 |
| | — | — | 11130.25 | 101.18 | 7.30 | 67.69 |
| | — | — | 13131.00 | 119.37 | — | — |
| Average | 82.16 | 0.75 | 7693.21 | 69.94 | 4.66 | 34.32 |
| SYN.PT3_Fab H.F.T2A.L | 340.65 | 6.19 | 3479.73 | 63.27 | 0.30 | 0.00 |
| (TAU_ITR188) | 678.10 | 12.33 | 800.91 | 14.56 | 10.50 | 0.00 |
| | 663.75 | 12.07 | 871.53 | 15.85 | 6.98 | 0.00 |
| | — | — | 743.96 | 13.53 | 3.48 | 0.00 |
| | — | — | 2094.76 | 38.09 | 4.09 | 0.00 |
| | — | — | 447.84 | 8.14 | 4.78 | 0.00 |
| Average | 560.83 | 10.20 | 1406.45 | 25.57 | 5.02 | 0.00 |
| CAG PT3_Fab H.F.T2A.L | 1077.65 | 19.59 | 2701.37 | 49.12 | 5.67 | 17.07 |
| (TAU_ITR180) | 703.40 | 12.79 | 7146.70 | 129.94 | 1.65 | 8.21 |
| | 894.50 | 16.26 | 4679.73 | 85.09 | 4.56 | 18.86 |
| | 903.90 | 16.43 | 2199.09 | 39.98 | 3.74 | 59.20 |
| | 833.20 | 15.15 | 2513.21 | 45.69 | 6.34 | 0.00 |
| | 1026.80 | 18.67 | 2270.84 | 41.29 | 4.48 | 51.66 |
| | — | — | — | — | 2.58 | 11.89 |
| Average | 906.58 | 16.48 | 3585.16 | 65.18 | 4.15 | 23.84 |
| GFAP.F(ab′)₂.HL.F2A | 0.00 | 0.00 | 274.00 | 2.49 | 0.63 | 0.00 |
| (TAU_ITR195) | 0.00 | 0.00 | 808.25 | 7.35 | 4.96 | 7.12 |
| | 0.00 | 0.00 | 3426.00 | 31.15 | 9.12 | 19.29 |
| | 0.00 | 0.00 | 414.75 | 3.77 | 2.58 | 3.39 |
| | 0.00 | 0.00 | 2556.00 | 23.24 | 7.52 | 15.07 |
| | — | — | 1057.75 | 9.62 | 4.00 | 10.46 |
| Average | 0.00 | 0.00 | 1422.79 | 12.93 | 4.80 | 9.22 |
| SYN.F(ab′)₂.HL.F2A | 66.60 | 0.61 | 195.00 | 1 77 | 4.54 | 0.00 |
| (TAU_ITR194) | — | — | 306.50 | 2.79 | 4.55 | 0.00 |
| | — | — | 243.50 | 2.21 | 4.05 | 0.00 |
| | 104.05 | 0.95 | 448.75 | 4.08 | 9.29 | 0.00 |
| | 106.40 | 0.97 | 364.75 | 3.32 | 7.72 | 0.00 |
| | 84.95 | 0.77 | 267.50 | 2.43 | 4.39 | 0.00 |
| Average | 90.50 | 0.82 | 304.33 | 2.77 | 5.76 | 0.00 |
| GFAP.PT3_Fab | 484.75 | 8.81 | 1300.38 | 23.64 | 7.36 | 11.21 |
| H.F.T2A.L (TAU_ITR189) | 234.65 | 4.27 | 3106.15 | 56.48 | 4.82 | 13.02 |
| | 654.35 | 11.90 | 1185.88 | 21.56 | 6.27 | 12.32 |
| | 443.60 | 8.07 | 5381.78 | 97.85 | 4.44 | 7.05 |
| | 956.35 | 17.39 | 3069.70 | 55.81 | 3.89 | 4.47 |
| | — | — | 1196.36 | 21.75 | 7.41 | 5.66 |
| Average | 554.74 | 10.09 | 2540.04 | 46.18 | 5.70 | 8.96 |
| GFAP.PT3_F(ab′)2 | 59.05 | 0.54 | 830.50 | 7.55 | 7.14 | 14.42 |
| H.hIgG3_3Hinge.F.T2A.L | 51.10 | 0.46 | 967.75 | 8.80 | 10.11 | 61.18 |
| (TAU_ITR192) | 68.10 | 0.62 | 704.75 | 6.41 | 8.79 | 12.44 |
| | 41.80 | 0.38 | 65.75 | 0.60 | 0.56 | 2.66 |
| | 90.45 | 0.82 | 1085.25 | 9.87 | 4.68 | 13.27 |
| | 52.50 | 0.48 | 704.75 | 6.41 | 5.01 | 35.44 |
| Average | 60.50 | 0.55 | 726.46 | 6.60 | 6.05 | 23.24 |
| SYN.PT3_F(ab′)2 | 33.90 | 0.31 | 310.25 | 2.82 | 4.31 | 0.00 |
| H.hIgG3_3Hinge.F.T2A.L | 120.95 | 1.10 | 311.75 | 2.83 | 3.08 | 0.00 |
| (TAU_ITR191) | 83.05 | 0.76 | 323.50 | 2.94 | 5.33 | 0.00 |
| | 113.65 | 1.03 | 164.00 | 1.49 | 4.54 | 0.00 |
| | 34.80 | 0.32 | 316.65 | 2.88 | 8.14 | 0.00 |
| | 102.25 | 0.93 | 296.25 | 2.69 | 6.80 | 0.00 |
| Average | 81.43 | 0.74 | 287.07 | 2.61 | 5.37 | 0.00 |
| | 45.30 | 0.41 | 2599.75 | 23.63 | 5.75 | 21.25 |
| CAGPT3_F(ab′)2 | 60.15 | 0.55 | 2990.75 | 27.19 | 7.73 | 81.32 |
| H.hIgG3_3Hinge.F.T2A.L | 64.75 | 0.59 | 2693.75 | 24.49 | 4.33 | 28.72 |
| (TAU_ITR190) | 54.70 | 0.50 | 2119.50 | 19.27 | 2.40 | 15.47 |
| | 53.80 | 0.49 | 1746.50 | 15.88 | 6.07 | 18.05 |
| | 74.05 | 0.67 | 3126.50 | 28.42 | 3.97 | 22.46 |
| Average | 58.79 | 0.53 | 2546.13 | 23.15 | 5.04 | 31.21 |
| Vehicle | 0.00 | 0.00 | 0.00 | 0.00 | 0.00 | 0.00 |

Consistent with the measurements in Table 177 and Table 178, higher PT3 expression levels were observed with Fab constructs (TAU_ITR180, TAU_ITR188, TAU_ITR189) compared to F(ab′)₂ constructs (TAU_ITR190, TAU_ITR191, TAU_ITR192, TAU_ITR193, TAU_ITR194, TAU_ITR195) and lowest with delivery of F(ab′)2 constructs. Among the Fab constructs, CSF PT3 fragment expression levels were the same regardless of the promoter used. In the group of mice dosed with the F(ab′)₂ T2A vectors (TAU_ITR190, TAU_ITR191, TAU_ITR192), no significant difference in the expression of PT3 was detected among the different promoters. In the serum, as expected, the CAG promoter appeared to drive the highest expression of PT3 in constructs (TAU_ITR180 (CAG PT3_Fab H.F.T2A.L), TAU_ITR190 (CAG PT3_F(ab′)2 H.hIgG3_3Hinge.F.T2A.L), TAU_ITR193 (CAG PT3_F(ab′)2 H.hIgG3_3Hinge.F.F2A.L)), whereas the SYN promoter had the least expression in constructs (TAU_ITR188 (SYN.PT3_Fab H.F.T2A.L), TAU_ITR191 (SYN.PT3_F(ab′)2 H.hIgG3_3Hinge.F.T2A.L), TAU_ITR194 (SYN PT3_F(ab′)2 H.hIgG3_3Hinge.F.F2A.L)). In the liver, all the constructs resulted in similar AAV biodistribution albeit at levels lower than seen in the CNS tissues. PT3 antibody expression was observed in mice transduced with CAG promoter constructs (TAU_ITR180 (CAG PT3_Fab H.F.T2A.L), TAU_ITR190 (CAG PT3_F(ab′)2 H.hIgG3_3Hinge.F.T2A.L),TAU_ITR193 (CAG PT3_F(ab′)2 H.hIgG3_3Hinge.F.F2A.L)) and to a lesser extent the GFAP promoter constructs (TAU_ITR189 (GFAP.PT3_Fab H.F.T2A.L), TAU_ITR192 (GFAP.PT3_F(ab′)2 H.hIgG3_3Hinge.F.T2A.L), TAU_ITR195 (GFAP PT3_F(ab′)2 H.hIgG3_3Hinge.F.F2A.L)). No PT3 expression was evident in the liver when driven by the synapsin promoter (TAU_ITR188 (SYN.Fab), TAU_ITR191 (SYN. F(ab′)2), TAU_ITR168 (SYN.scFv)).

The statistical significance of the data in Table 179 was analyzed using one-way ANOVA-Tukey's multiple comparison test. PT3 expression (for both ng/ml and nM) was significantly higher (p<0.0005) in the CSF of the former of the following pairs when compared to the latter, (a) CAG PT3_Fab H.F.T2A.L (TAU_ITR180) compared to CAG PT3_F(ab′)2 H.hIgG3_3Hinge.F.T2A.L (TAU_ITR190), ssAAV CAG PT3 VL G4S-3 VH HA rBGpA (TAU_ITR162), GFAP.PT3_Fab H.F.T2A.L (TAU_ITR189) or SYN.PT3_Fab H.F.T2A.L (TAU_ITR188) (b) GFAP.PT3_Fab H.F.T2A.L (TAU_ITR189) compared to GFAP.PT3_F(ab′)2 H.hIgG3_3Hinge.F.T2A.L (TAU_ITR192) or ssAAV GFAP PT3 VL G4S-3 VH HA rBGpA (TAU_ITR165); (c) SYN.PT3_Fab H.F.T2A.L (TAU_ITR188) compared to SYN.PT3_F(ab′)2 H.hIgG3_3Hinge.F.T2A.L (TAU_ITR191), or ssAAV SYN PT3 VL G4S-3 VH HA rBGpA (TAU_ITR168). PT3 expression (nM) was also significantly higher (p<0.005 and <0.05 respectively) in the CSF of the former of the following pairs (a) CAG PT3_F(ab′)2 H.hIgG3_3Hinge.F.T2A.L (TAU_ITR190), compared to ssAAV CAG PT3 VL G4S-3 VH HA rBGpA (TAU_ITR162) (b) ssAAV CAG PT3 VL G4S-3 VH HA rBGpA (TAU_ITR162) compared to ssAAV SYN PT3 VL G4S-3 VH HA rBGpA (TAU_ITR168).

In the serum, PT3 expression (ng/ml) was significantly higher with a value of P<0.0005 in the serum of rodents transduced with CAG PT3_Fab H.F.T2A.L (TAU_ITR180) compared to ssAAV CAG PT3 VL G4S-3 VH HA rBGpA (TAU_ITR162). Higher PT3 expression (ng/ml) with a significance of p value <0.005 was observed in the PT3 in the serum of rodents treated with the former constructs in the following pairs compared to the latter constructs (a) CAG PT3_Fab H.F.T2A.L (TAU_ITR180) compared to CAG PT3_F(ab′)2 H.hIgG3_3Hinge.F.T2A.L (TAU_ITR190) (b) CAG PT3_F(ab′)2 H.hIgG3_3Hinge.F.T2A.L (TAU_ITR190) compared to SYN.PT3_F(ab′)2 H.hIgG3_3Hinge.F.T2A.L (TAU_ITR191) (c) ssAAV CAG PT3 VL G4S-3 VH HA rBGpA (TAU_ITR162) compared to ssAAV SYN PT3 VL G4S-3 VH HA rBGpA (TAU_ITR168) (d) GFAP.PT3_Fab H.F.T2A.L (TAU_ITR189) compared to ssAAV GFAP PT3 VL G4S-3 VH HA rBGpA (TAU_ITR165). Higher PT3 expression (ng/ml) (p value <0.05) was observed in the PT3 in the serum of rodents transduced with the former constructs in the following pairs compared to the latter constructs (a) CAG PT3_F(ab′)2 H.hIgG3_3Hinge.F.T2A.L (TAU_ITR190) compared to GFAP.PT3_F(ab′)2 H.hIgG3_3Hinge.F.T2A.L (TAU_ITR192) and (b) GFAP.PT3_Fab H.F.T2A.L (TAU_ITR189) compared to GFAP.PT3_F(ab′)2 H.hIgG3_3Hinge.F.T2A.L (TAU_ITR192). In comparing the PT3 expression (nM), the expression was significantly higher (p<0.0005) in CAG PT3_Fab H.F.T2A.L (TAU_ITR180) compared to ssAAV CAG PT3 VL G4S-3 VH HA rBGpA (TAU_ITR162) or SYN.PT3_Fab H.F.T2A.L (TAU_ITR188). A significance of p value <0.005 was obtained when the PT3 expression (nM) in the serum was compared between the former of the following pairs compared to the latter (a) CAG PT3_Fab H.F.T2A.L (TAU_ITR180) compared to CAG PT3_F(ab′)2 H.hIgG3_3Hinge.F.T2A.L (TAU_ITR190); (b) ssAAV CAG PT3 VL G4S-3 VH HA rBGpA (TAU_ITR162) compared to ssAAV SYN PT3 VL G4S-3 VH HA rBGpA (TAU_ITR168) (c) GFAP.PT3_Fab H.F.T2A.L (TAU_ITR189) compared to GFAP.PT3_F(ab′)2 H.hIgG3_3Hinge.F.T2A.L (TAU_ITR192) (d) GFAP.PT3_F(ab′)2 H.hIgG3_3Hinge.F.T2A.L (TAU_ITR192) compared to ssAAV GFAP PT3 VL G4S-3 VH HA rBGpA (TAU_ITR165). In general, antibody levels were highest following delivery of constructs encoding Fab fragments, followed by F(ab′)2. Expression driven by the CAG promoter resulted in the highest expression of antibody fragment in the serum, as compared to when driven by cell-specific promoters.

Immunohistochemistry (using DAB and an anti-kappa light chain primary antibody) was performed on brain tissue of mice treated with an IV bolus injection of VOY101.PT3 AAV particles comprising viral genomes encoding (Fab′)₂ or Fab, driven by CAG, SYN or GFAP promoters 28 days prior.

Comparing the PT3 Fab fragment-based constructs namely GFAP.Fab.HL.T2A (TAU_ITR189), SYN.Fab.HL.T2A (TAU_ITR188), CAG.Fab.HL.T2A (TAU_ITR180), a broad distribution of PT3 Fab fragment was observed in mice dosed with PT3 Fab vectors under CAG or cell specific promoters. Broad distribution of PT3 Fab fragment was observed from anterior to posterior coronal sections of the brain (e.g., hippocampus) of mice dosed with CAG or cell specific promoter.

PT3 F(ab′)2 T2A fragment-based constructs, namely GFAP.F(ab′)2.HL.T2A (TAU_ITR192), SYN.F(ab′)2.HL.T2A (TAU_ITR191), and CAG.F(ab′)2.HL.T2A (TAU_ITR190), also showed a broad distribution of PT3 F(ab′)2 fragment.

Strong staining was observed in multiple CNS regions in all groups dosed with vectorized Fab, but overall, CAG PT3 Fab or PT3 F(ab′)2 (F2A vectors) showed stronger signal in the brain when compared to the other constructs in Table 175 with cell specific promoters. Vehicle treated control animals showed low background levels of staining.

Taken together, these data show that constructs operably linked to the CAG promoter namely TAU_ITR180 (CAG PT3_Fab H.F.T2A.L), TAU_ITR190 (CAG PT3_F(ab′)2 H.hIgG3_3Hinge.F.T2A.L),TAU_ITR193 (CAG PT3_F(ab′)2 H.hIgG3_3Hinge.F.F2A.L) showed stronger signal in the brain when compared to those wherein expression is driven by cell specific promoters namely constructs TAU_ITR188 (SYN.PT3_Fab H.F.T2A.L).TAU_ITR191 (SYN.PT3_F(ab′)2 H.hIgG3_3Hinge.F.T2A.L), TAU_ITR194 (SYN PT3_F(ab′)2 H.hIgG3_3Hinge.F.F2A.L), TAU_ITR189 (GFAP.PT3_Fab H.F.T2A.L), TAU_ITR192 (GFAP.PT3_F(ab′)2 H.hIgG3_3Hinge.F.T2A.L), TAU_ITR195 (GFAP PT3_F(ab′)2 H.hIgG3_3Hinge.F.F2A.L).

The cell-specificity of the expression of the constructs described in Table 175 was also evaluated. Among the PT3 Fab constructs GFAP.Fab.HL.T2A (TAU_ITR189). SYN.Fab.HL.T2A (TAU_ITR188), and CAG.Fab.HL.T2A (TAU_ITR180) PT3 expression was detected in cells with neuronal and glial cell morphology in the hippocampus, cortex, thalamus and the spinal cord when a CAG promoter was used. PT3 Fab expression was most evident in cells with astrocytic morphology in cortex. PT3 Fab fragment expression was also detected in cells with neuronal or glial cell morphology in indicated brain regions when Syn or GFAP promoters were used, respectively. For viral genomes comprising a CAG promoter, PT3 Fab fragment expression was most evident in cells with astrocytic morphology in cortex. Further, PT3 Fab T2A constructs GFAP.Fab.HL.T2A (TAU_ITR189), SYN.Fab.HL.T2A (TAU_ITR188), and CAG.Fab.HL.T2A (TAU_ITR180) appeared to have higher expression when compared to other F(ab′)2 constructs (GFAP.F(ab′)2.HL.T2A (TAU_ITR192), SYN.F(ab′)2.HL.T2A (TAU_ITR191) and CAG.F(ab′)2.HL.T2A (TAU_ITR190).

When all data on delivery of viral genomes expressing antibody fragments are considered in aggregate, Fab.HL.T2A format appears to result in the highest expression of antibody fragment in target tissue.

Further, in comparing expression of anti-tau antibody fragments after IV delivery of VOY101 particles, brain levels were substantially higher than the level of expression seen after IV delivery of full length antibody.

In conclusion, these data showed that intravenous delivery of AAV particles (e.g., Voy101) encoding anti-tau antibodies or antibody fragments resulted in widespread vector genome biodistribution and therapeutically relevant antibody levels in mouse CNS tissues.

### VII. Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit in its broader aspects.

While the present disclosure has been described at some length and with some particularity with respect to the several described embodiments, it is not intended that it should be limited to any such particulars or embodiments or any particular embodiment, but it is to be construed with reference to the appended claims so as to provide the broadest possible interpretation of such claims in view of the prior art and, therefore, to effectively encompass the intended scope.

1. An AAV viral genome comprises:
a. a 5′ inverted terminal repeat (ITR) sequence region;
b. a promoter sequence region;
c. at least one antibody polynucleotide, wherein said at least one antibody polynucleotide comprises a polynucleotide sequence with at least 90% identity, or encodes an amino acid sequence with at least 90% identity, to a sequence selected from the group consisting of SEQ ID NO: 1740-1989, 2241-2243, and 2169-2170; and
d. a 3′ ITR sequence region.

2. The AAV viral genome of claim 1 comprising:
an exon sequence regions selected from the group consisting of SEQ ID NO: 2090-2094;
an intron sequence region selected from the group consisting of SEQ ID NO: 2095-2105, 2240 and 2256-2258;
a signal sequence region selected from the group consisting of SEQ ID NO: 1740, 1741, 1861, 2106-2117 and 2241;
a tag sequence region selected from the group consisting of SEQ ID NO: 2118-2121 and 2255; and/or
a filler sequence region selected from the group consisting of SEQ ID NO: 2125 and 2126.

3-6. (canceled)

7. The AAV viral genome of claim 1, comprising two antibody polynucleotides.

8. The AAV viral genome of claim 7, wherein the two antibody polynucleotides are separated by a linker sequence selected from the group consisting of SEQ ID NO: 1724-1739, 2244-2254 and 2259.

9. The AAV viral genome of claim 7 wherein a first antibody polynucleotide encodes an antibody heavy chain or a fragment thereof.

10. The AAV viral genome of claim 7 wherein a second polynucleotide encodes an antibody light chain or a fragment thereof.

11. The AAV viral genome of claim 1 comprising more than two antibody polynucleotides.

12. The AAV viral genome of claim 8, encoding from 5′ to 3′;
an antibody heavy chain, a linker sequence, and an antibody light chain; or
an antibody light chain, a linker sequence, and an antibody heavy chain.

13. (canceled)

14. An AAV particle comprising the AAV viral genome of claim 1.

15. The AAV particle of claim 14, wherein the sequence of the AAV viral genome is selected from the group consisting of SEQ ID NO: 1990-2075, 2137-2168, 2171-2237 and 2260-2321.

16. The AAV particle of claim 14, comprising an AAV serotype selected from the group consisting of VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2 variant, AAV2/3 variant, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9K449R, AAV9.11, AAV9.13, AAV9.16, AAV9.24, AAV9.45, AAV9.47, AAV9.61, AAV9.68, AAV9.84, AAV9.9, AAV10, AAV11, AAV12 and variants thereof.

17. (canceled)

18. The AAV particle of claim 16, where the amino acid sequence of VOY101 comprises SEQ ID NO: 1.

19. The AAV particle of claim 14, comprising an AAV serotype of AAV2 or an AAV2 variant, AAV5 or an AAV5 variant, or AAV9 or an AAV9 variant.

20-23. (canceled)

24. A pharmaceutical composition comprising the AAV particle of claim 14.

25. A method of producing a functional antibody in a subject in need thereof, comprising administering to said subject the pharmaceutical composition of claim 24.

26. The method of claim 25, wherein the level or amount of the functional antibody in the target cell or tissue after administration to the subject is from about 0.001 ug/mL to 100 mg/mL.

27. The method of claim 25, wherein the functional antibody is encoded by the at least one antibody polynucleotide of the viral genome within said AAV particle.

28. (canceled)

29. A method for treating tauopathy in a subject in need, comprising administering to said subject a therapeutically effective amount of the pharmaceutical composition of claim 24.

30. The method of claim 29 comprising administering the AAV particle by a delivery route selected from the group consisting of intravenous intraparenchymal, intracerebroventricular, and intracisternal.

31-36. (canceled)

37. The methods of claim 29, wherein the tauopathy is selected from the group consisting of Alzheimer's disease (AD), frontotemporal dementia and parkinsonism linked to chromosome 17 (FTDP-17), Frontotemporal lobar degeneration (FTLD), Frontotemporal dementia, chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis and other tau associated disease.

38-39. (canceled)

40. The AAV genome of claim 1, further comprising a polyadenylation (polyA) signal sequence region, and wherein:
the 5′ ITR sequence region is selected from the group consisting of SEQ ID NO: 2076 and 2077;
the promoter sequence region is selected from the group consisting of SEQ ID NO: 2080-2089 and 2238-2239;
the 3′ ITR sequence region is selected from the group consisting of SEQ ID NO: 2078 and 2079; and/or
the polyA signal sequence region is selected from the group consisting of SEQ ID NO: 2122-212.
